(19) United States
(12) Patent Application Publication
(10) Pub. No.: US 2024/0043420 A1
(43) Pub. Date: Feb. 8, 2024

(54) 7-AZAINDOLE COMPOUNDS FOR INHIBITION OF BCR-ABL TYROSINE KINASES

(71) Applicant: EnLiven Therapeutics, Inc., Boulder, CO (US)

(72) Inventors: Joseph P. LYSSIKATOS, Boulder, CO (US); Samuel KINTZ, Boulder, CO (US); Li REN, Superior, CO (US)

(21) Appl. No.: 18/030,205

(22) PCT Filed: Oct. 4, 2021
(86) PCT No.: PCT/US2021/071692

§ 371 Date: Apr. 4, 2023

Related U.S. Application Data
(60) Provisional application No. 63/087,780, filed on Oct. 5, 2020.

(51) Int. Cl. C07D 471/04; C07D 519/00

(52) U.S. Cl. CPC C07D 471/04; C07D 519/00

(57) ABSTRACT

The present disclosure relates to compounds and compositions for inhibition of Bcr-Abl tyrosine kinases, methods of preparing said compounds and compositions, and their use in the treatment of various cancers, such as chronic myeloid leukemia (CML).

### CROSS-REFERENCE TO RELATED APPLICATION

This application claims priority to and the benefit of U.S. Provisional Application No. 63/087,780, filed on Oct. 5, 2020, the disclosure of which is incorporated herein by reference in its entirety.

### FIELD OF THE INVENTION

Provided herein are compounds and compositions for inhibition of Bcr-Abl tyrosine kinases, methods of preparing said compounds and compositions, and their use in the treatment of various cancers, such as chronic myeloid leukemia (CML).

### BACKGROUND

The cytogenetic abnormality known as the Philadelphia chromosome is highly associated with the occurrence of a number of hematological malignancies, including a majority of chronic myeloid leukemias (CML) and a subset of acute lymphoblastic leukemias (Ph+ALL). The Philadelphia chromosome is a product of a translocation between the breakpoint cluster region (BCR) gene on chromosome 22 and the Abelson (ABL) tyrosine kinase gene on chromosome 9, resulting in the oncogenic fusion gene product Bcr-Abl. The resultant fusion protein is both overexpressed and harbors constitutive kinase activity that then drives the activation of a number of intracellular signaling cascades to induce the uncontrolled cell growth, division and survival associated with oncogenic transformation. Accordingly, therapeutic intervention employing inhibitors of the Bcr-Abl tyrosine kinase represents a cornerstone of the current treatment paradigm for patients with Philadelphia-positive neoplastic disorders.

Imatinib (STI-571), a small molecule Bcr-Abl tyrosine kinase inhibitor (Bcr-Abl TKI), was developed as a highly effective treatment for CML in the early 1990s and is still employed today as a first line treatment for CML. However, in more aggressive cases of CML, patients often relapse due to the emergence of resistance. The primary mechanism of this resistance derives from a variety of on-target genetic alterations that drives either aberrant overexpression of the Bcr-Abl fusion or, more commonly, introduce amino acid mutations within the Abl kinase domain that reduce imatinib's binding affinity for the active site thereby markedly reducing its inhibitory activity. These alterations can either appear stochastically and represent a sub-population within the initial tumor cell population or arise under the selective pressure of inhibitor treatment. One of the predominant on-target Bcr-Abl resistance mutations derives from point mutations that introduce an isoleucine residue for a threonine at position 315 within the Abl kinase domain (T315I) also known as the ‘gatekeeper’ position. In addition to imatinib, this mutant form of BCR-Abl is profoundly resistant to all second generation Bcr-Abl TKIs (Nilotinib, Dasatinib, Bosutinib, Radotinib). Currently, there exists only one therapeutic option for patients harboring a T315I mutation—the third line Bcr-Abl TKI, Ponatinib. While effective at treating patients with T315I CML, ponatinib suffers from poor selectivity for Bcr-Abl versus a number of other protein kinases. Accordingly, ponatinib has been reported to elicit significant dose-limiting toxicities, which then limits its ability to effectively engage the target to achieve clinical efficacy.

Besides on- or off-target resistance, intolerance to Bcr-Abl TKIs also represents a major clinical challenge. The doses of more than 50% of Ph+leukemia patients require modification due to adverse events. In fact, approximately 30% of patients are compelled to dose reduce within the first 6 months of treatment. These drug-related side effects appear early in the course of treatment and, while manageable in most cases, toxicities persist, significantly impacting the patients' quality of life, resulting in decreased compliance. Accordingly, around 40% of patients discontinue first and second generation Bcr-Abl TKIs within the first 5 years of treatment. All of the currently approved Bcr-Abl targeted therapies inhibit other tyrosine kinases, which can lead to potentially debilitating side effects. Specifically, potent inhibition of VEGFRs, PDGFRs, c-Kit and/or the c-Src family can lead to dose-limiting side effects in patients. To address these adverse effects, dose reductions, dose interruptions, and even dose discontinuations are often required during the course of therapy, however such treatment regimens ultimately result in suboptimal therapeutic benefit.

Accordingly, there remains a substantial unmet medical need for Bcr-Abl TKIs with improved selectivity to improve tolerability and enhanced potency against the wide array of resistance mechanisms in Philadelphia-positive disorders.

### SUMMARY OF THE INVENTION

Provided herein are compounds and compositions that selectively inhibit Bcr-Abl tyrosine kinases and that are useful for treating disorders mediated by Bcr-Abl tyrosine kinases.

In one aspect, provided herein is a compound of formula (I),

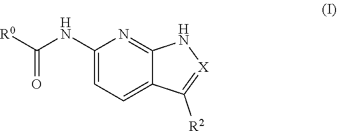

or a pharmaceutically acceptable salt, solvate, hydrate, or co-crystal thereof, or a mixture of any of the foregoing, wherein:
X is N or CR⁸;
R⁰ is a group

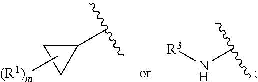

m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, —O—C₁-C₃ alkylene-NR⁴R⁵, —O—C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(4- to 8-membered heterocycloalkylene)-(C₀-C₂ alkylene)-NR⁴R⁵, C₁-C₂ alkylene-(4- to 8-membered heterocycloalkylene)-(C₀-C₂ alkylene)-NR^{4′}R⁵, C₁-C₂ alkylene-(C₃-C₇ heterocycloalkylene)-(C₀-C₂ alkylene)-NR⁴R⁵ or C₁-C₂ alkylene-(C₃-C₇ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl,
or
two R¹ are taken together with the carbon atom or carbon atoms to which they are attached to form a 3- to 7-membered heterocyclic ring, wherein the heterocyclic ring contains nitrogen atom and wherein the nitrogen atom is optionally substituted with C₁-C₃ alkyl;
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(4- to 8-membered heterocycloalkyl), C₁-C₃ alkylene-(C₃-C₇ heterocycloalkyl), C₁-C₃ alkylene-(4- to 8-membered heterocycloalkylene)-(C₀-C₂ alkylene)-NR⁴R⁵, C₁-C₃ alkylene-(4- to 8-membered heterocycloalkylene)-(C₀-C₂ alkylene)-NR^{4′}R^{5′}, (4- to 8-membered heterocycloalkylene)-(C₀-C₂ alkylene)-NR⁴R⁵, C₁-C₃ alkylene-(C₃-C₇ heterocycloalkylene)-(C₀-C₂ alkylene)-NR⁴R⁵, C₁-C₃ alkylene-(C₃-C₇ heterocycloalkylene)-(C₀-C₂ alkylene)-NR^{4′}R^{5′}, or (C₃-C₇ heterocycloalkylene)-(C₀-C₂ alkylene)-NR⁴R⁵, wherein the alkyl, alkylene, cycloalkyl, cycloalkylene, heterocycloalkyl, and heterocycloalkylene moieties in R³ are optionally substituted with 1-3 fluorine atoms, 1-3 CN groups and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, 4- to 8-membered heterocycloalkyl, or C₃-C₇ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each pair of R^{4′} and R^{5′} taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O)ₙC₁-C₃ alkyl, or S(O)ₙC₃-C₆ cycloalkyl, wherein n is an integer from 0 to 2;
each R⁷ is independently —H, C₁-C₃ alkyl, C₁-C₃ haloalkyl, C₃-C₆ cycloalkyl, or C₁-C₃-alkylene-C₃-C₆ cycloalkyl, wherein said C₁-C₃ alkyl is optionally substituted with 1-6 deuterium atoms; and
R⁸ is —H, —F, or C₁-C₃ alkyl.

In another aspect, provided herein is a compound of formula (I),

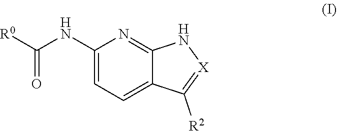

or a pharmaceutically acceptable salt, solvate, hydrate, or co-crystal thereof, or a mixture of any of the foregoing, wherein:
X is N or CR¹;
R⁰ is a group

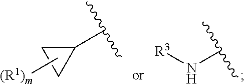

m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl,
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(C₄-C₆ heterocycloalkyl), C₁-C₃ alkylene-(C₄-C₆ heterocycloalkylene)-(C₀-C₂ alkylene)-NR⁴R⁵ or C₁-C₃ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O)ₙC₁-C₃ alkyl, or S(O)ₙC₃-C₆ cycloalkyl,
wherein n is an integer from 0 to 2;
each R⁷ is independently —H, C₁-C₃ alkyl, C₁-C₃ haloalkyl, or C₃-C₆ cycloalkyl, wherein said C₁-C₃ alkyl is optionally substituted with 1-6 deuterium atoms; and
R⁸ is —H, —F, or C₁-C₃ alkyl.

In some embodiments, the compound of formula (I) is a compound of formula (I-A)

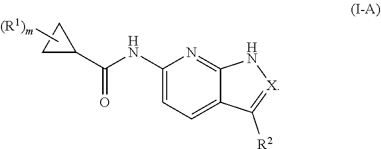

In some embodiments, the compound of formula (I) is a compound of formula (I-A-i) or formula (I-A-ii)

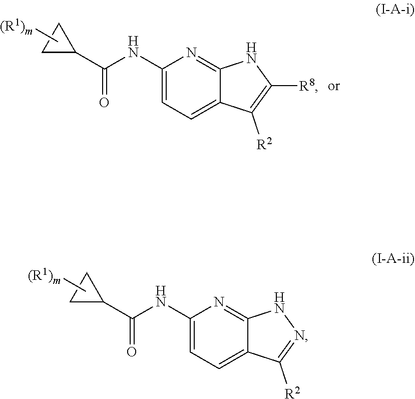

wherein:
m is an integer from 0 to 2;
each R¹ is independently —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
R² is C₆-C₁₄ aryl or 5-to-10-membered heteroaryl, wherein said 5-to-10-membered heteroaryl is selected from the group consisting of

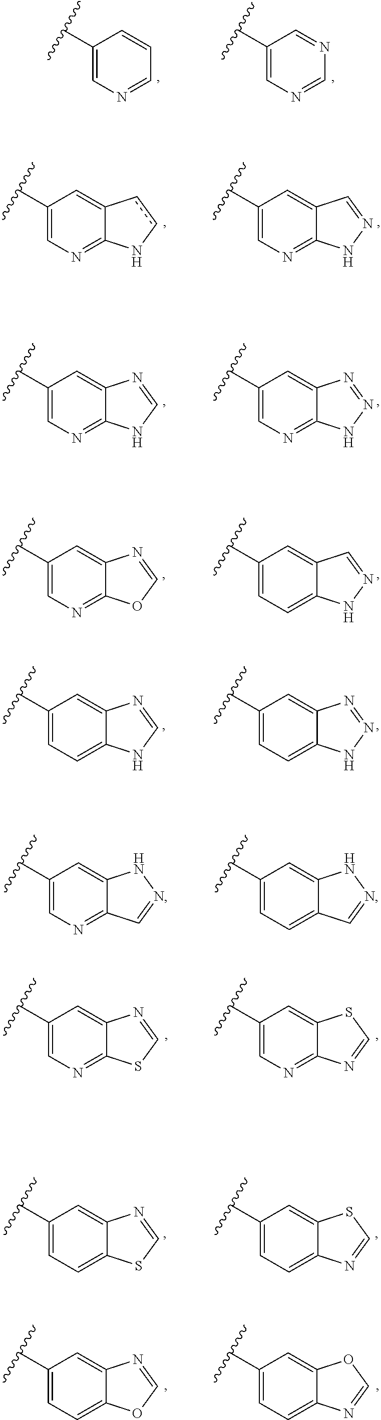

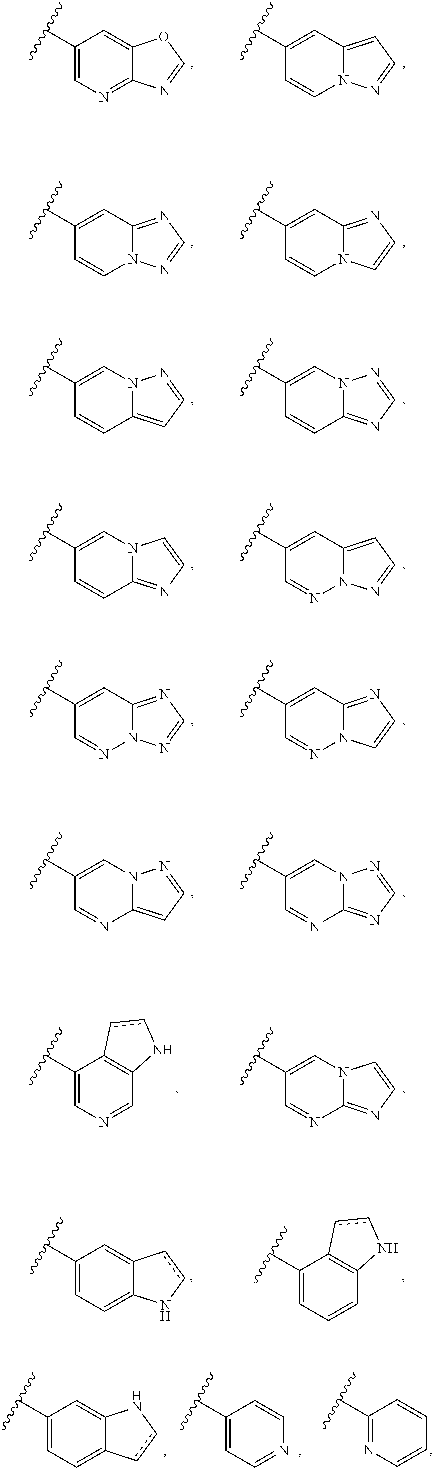

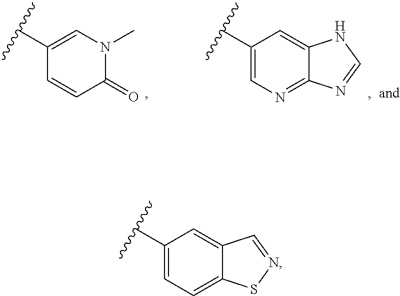

wherein [symbol] indicates a single or double bond, and wherein the C₆-C₁₄ aryl and 5-to-10-membered heteroaryl are optionally substituted with 1-5 R⁶ groups;
each R⁴ is independently —H, C₁-C₃ alkyl, C₃-C₆ cycloalkyl, C₂-C₃ haloalkyl, C₂-C₃ alkylene-CN, or C₂-C₃ heteroalkyl;
each R⁵ is independently —H, C₁-C₃ alkyl, C₃-C₆ cycloalkyl, C₂-C₃ haloalkyl, C₂-C₃ alkylene-CN, or C₂-C₃ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, C₁-C₃ alkyl, —CF₂H, —CF₃, C₃-C₆ cycloalkyl, or —CN;
each R⁷ is independently —H, C₁-C₃ alkyl, —CD₃, —CF₂H, —CF₃, or C₃-C₆ cycloalkyl; and
R⁸ is —H, —F, or —CH₃.

In some embodiments, which may be combined with any of the preceding embodiments, each R¹ is independently —F, C₁-C₃ alkylene-NR^{4′}R^{5′}, C₁-C₃ alkylene-OH, or C₀-C₃ alkylene-CN, wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl.

In other embodiments, which may be combined with any of the preceding embodiments, R² is phenyl,

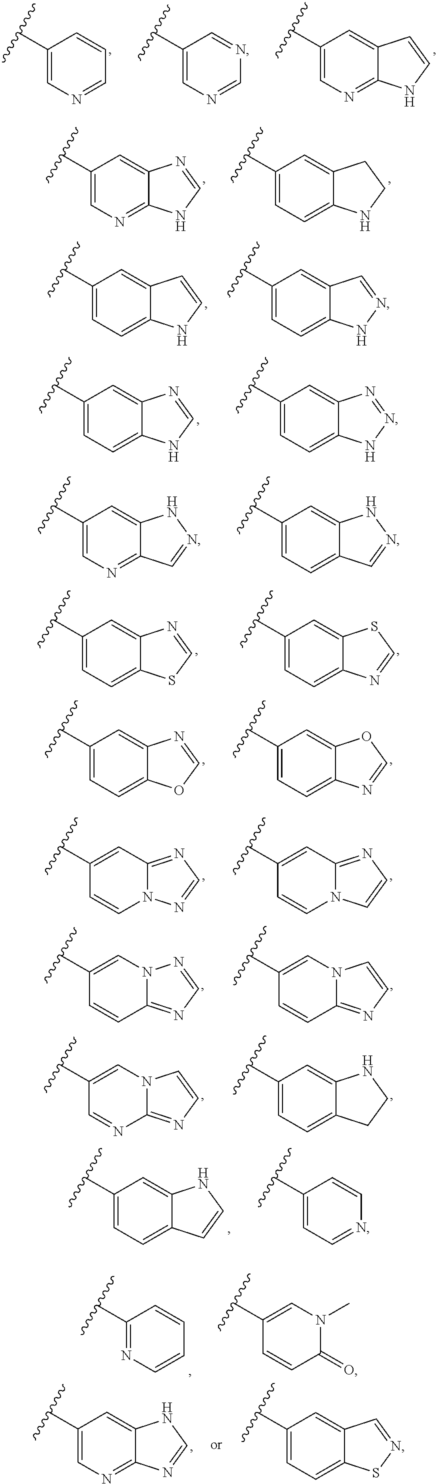

each of which is optionally substituted with 1-5 R⁶ groups.

In yet other embodiments, which may be combined with any of the preceding embodiments, each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl; and each R⁷ is independently —H, C₁-C₂ alkyl, —CD₃, C₁-C₂ haloalkyl, or C₃ cycloalkyl.

In still yet other embodiments, which may be combined with any of the preceding embodiments, each R¹ is independently —F, C₁-C₃ alkylene-NR^{4′}R^{5′}, C₁-C₃ alkylene-OH, or C₀-C₃ alkylene-CN, wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl;
R² is phenyl,

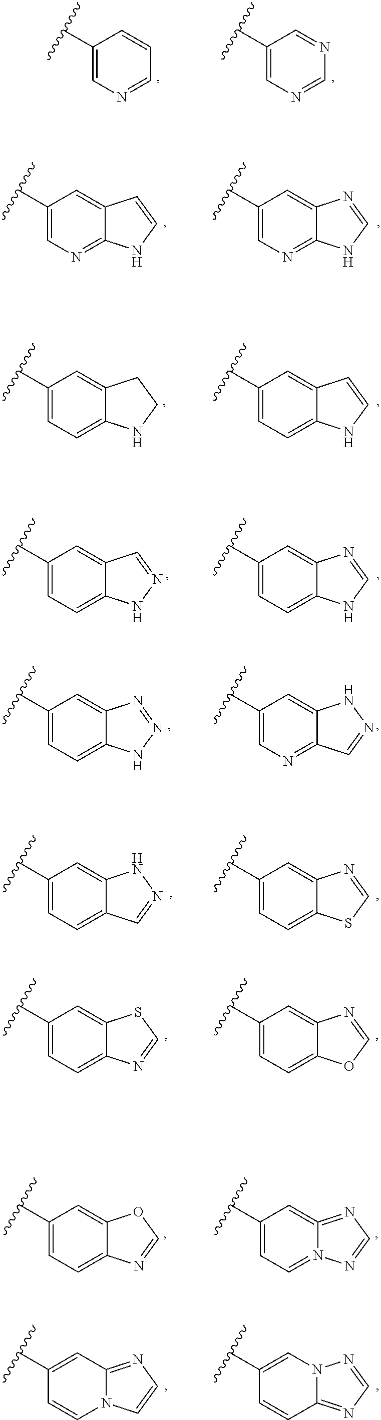

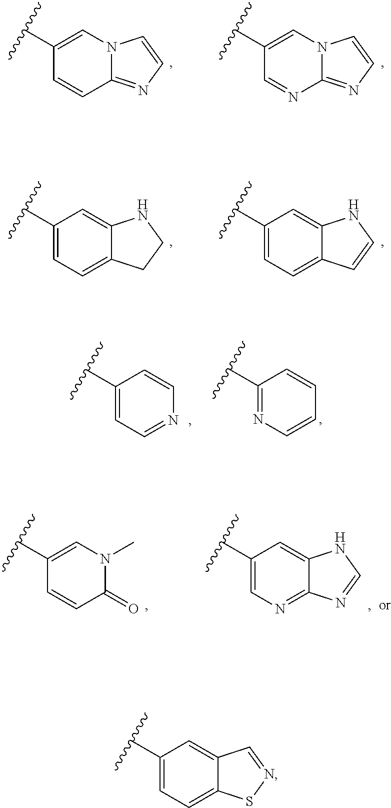

each of which is optionally substituted with 1-5 R⁶ groups;
each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl; and
each R⁷ is independently —H, C₁-C₂ alkyl, —CD₃, C₁-C₂ haloalkyl, or C₃ cycloalkyl.

In still other embodiments, which may be combined with any of the preceding embodiments, each R¹ is independently F,

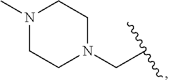 [SMILES: *CN1CCN(C)CC1]

—CH₂OH, —CH₂CH₂OH, —CN, or —CH₂CN. In some embodiments, each R⁶ is independently —F, —Cl, —OH, —OCH₃, —OCH₂CH₃, —OCF₃, —OCF₂H, —OCH₂CF₃, —OCD₃, cyclopropyloxy, —NH₂, —NHCH₃, —N(CH₃)₂, —CH₃, —CF₃, or —CN.

In other embodiments of the present aspect, the compound of formula (I) is a compound of formula (I-B)

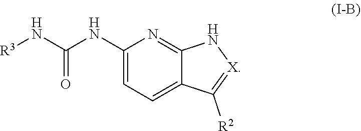

In some embodiments of the foregoing, the compound of formula (I) is a compound of formula (I-B-i) or formula (I-B-ii)

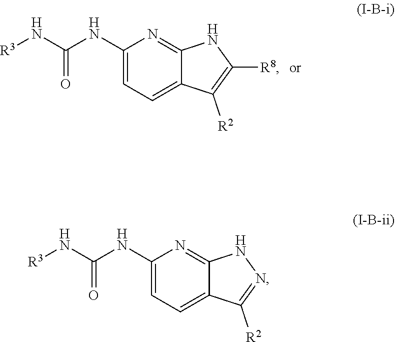

wherein:
R² is C₆-C₁₄ aryl or 5-to-10-membered heteroaryl, wherein said 5-to-10-membered heteroaryl is selected from the group consisting of

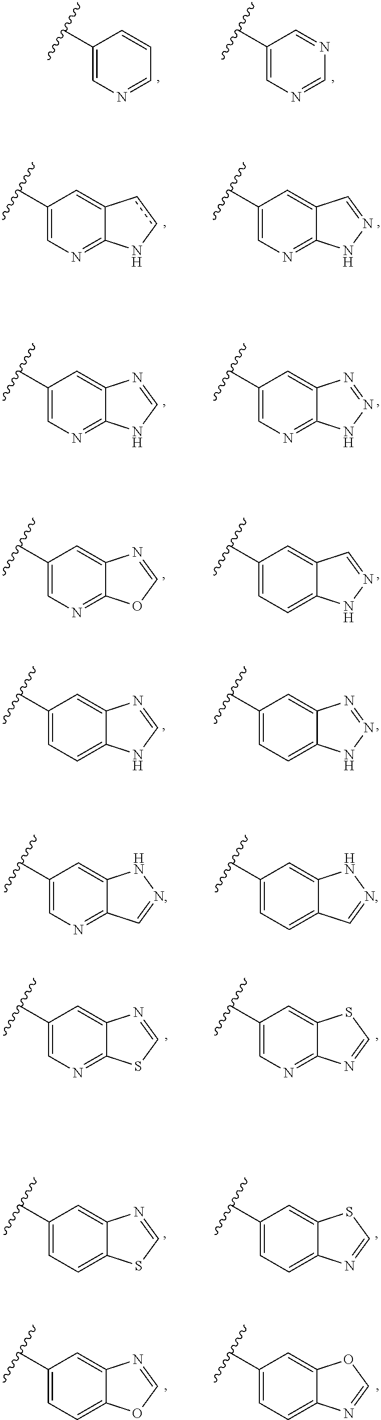

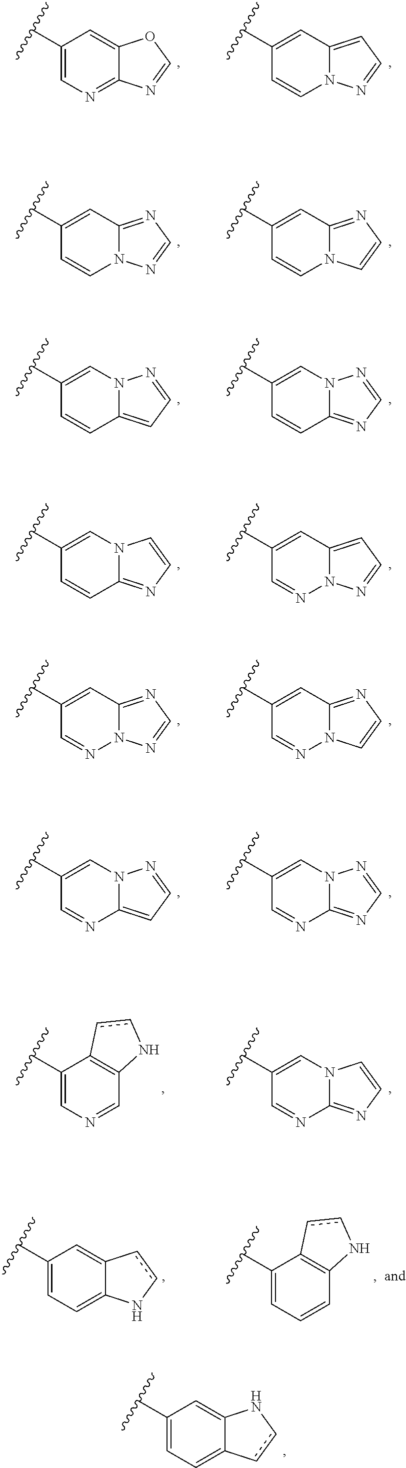

wherein [symbol] indicates a single or double bond, and wherein the C₆-C₁₄ aryl and 5-to-10-membered heteroaryl are optionally substituted with 1-5 R⁶ groups;
R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-OH, C₁-C₃ alkylene-CN, C₁-C₂ alkylene-(C₃-C₆ cycloalkyl), C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-O—(C₁-C₂ alkylene)-NR⁴R⁵, C₁-C₂ alkylene-(C₃-C₆ cycloalkylene)-(C₀-C₂ alkylene)-OH, C₁-C₂ alkylene-(C₄-C₆ heterocycloalkyl), C₁-C₂ alkylene-(C₄-C₆ heterocycloalkylene)-(C₀-C₂ alkylene)-NR⁴R⁵, or C₁-C₂ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl;
each R⁴ is independently —H, C₁-C₃ alkyl, C₃-C₆ cycloalkyl, C₂-C₃ haloalkyl, C₂-C₃ alkylene-CN, or C₂-C₃ heteroalkyl;
each R⁵ is independently —H, C₁-C₃ alkyl, C₃-C₆ cycloalkyl, C₂-C₃ haloalkyl, C₂-C₃ alkylene-CN, or C₂-C₃ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, C₁-C₃ alkyl, —CF₂H, —CF₃, C₃-C₆ cycloalkyl, or —CN;
each R⁷ is independently —H, C₁-C₃ alkyl, —CD₃, —CF₂H, —CF₃, or C₃-C₆ cycloalkyl; and
R⁸ is —H, —F, or —CH₃.

In some embodiments, which may be combined with any of the preceding embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, or C₁-C₂ alkylene-(C₄-C₆ heterocycloalkyl), wherein each heterocyclic nitrogen atom, if present, is independently optionally substituted with C₁-C₃ alkyl or C₄-C₆ heterocycloalkyl, wherein the R⁴ and R⁵ of R³ are independently —H or C₁-C₃ alkyl, and the R^{4′} and R^{5′} of R³ taken together with the nitrogen atom to which they are attached form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl. In other embodiments, which may be combined with any of the preceding embodiments, R² is phenyl,

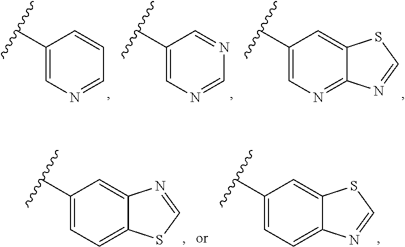

each of which is optionally substituted with 1-5 R⁶ groups. In still other embodiments, which may be combined with any of the preceding embodiments, each R⁶ is independently halogen or —OR⁷; and each R⁷ is independently C₁-C₂ alkyl or C₃ cycloalkyl. In yet other embodiments, which may be combined with any of the preceding embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, or C₁-C₂ alkylene-(C₄-C₆ heterocycloalkyl), wherein each heterocyclic nitrogen atom, if present, is independently optionally substituted with C₁-C₃ alkyl or C₄-C₆ heterocycloalkyl, wherein the R⁴ and R⁵ of R³ are independently —H or C₁-C₃ alkyl, and the R^{4′} and R^{5′} of R³ taken together with the nitrogen atom to which they are attached form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl; R² is phenyl,

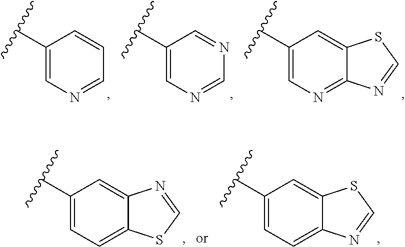

each of which is optionally substituted with 1-5 R⁶ groups; each R⁶ is independently halogen or —OR⁷; and each R⁷ is independently C₁-C₂ alkyl or C₃ cycloalkyl. In still yet other embodiments, which may be combined with any of the preceding embodiments, R³ is

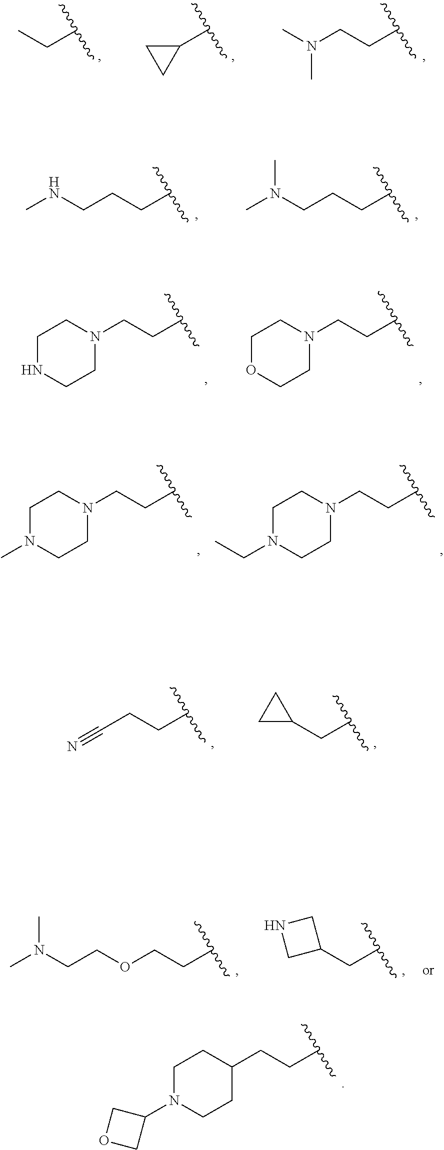

In still further embodiments, each R⁶ is independently —F, —OCH₃, or cyclopropyloxy. In yet other embodiments, which may be combined with any of the preceding embodiments, R¹ is H.

Also provided herein is a compound which is selected from the group consisting of

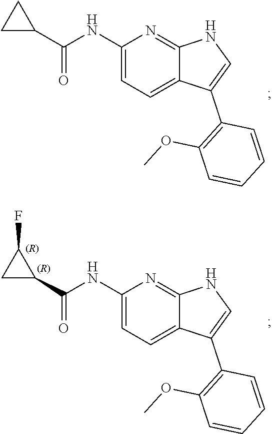

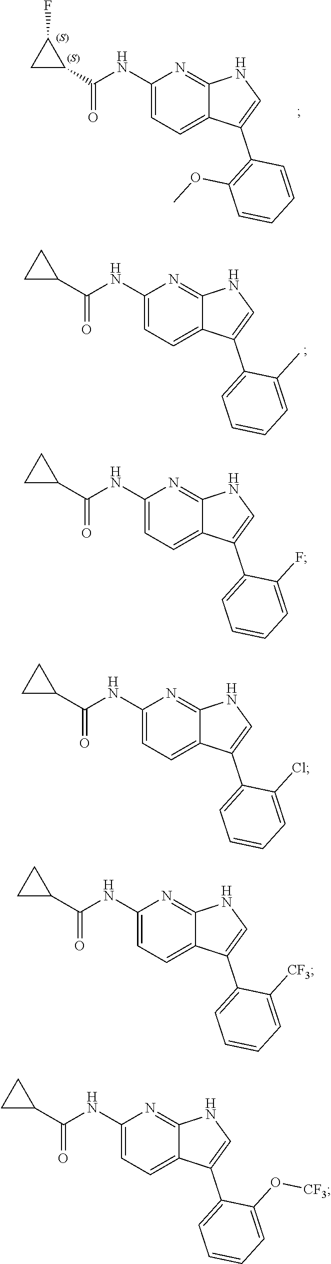

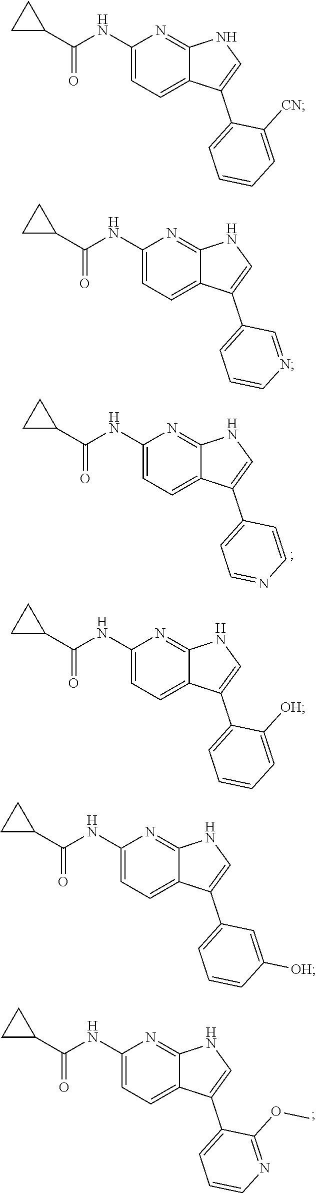 [SMILES: N#Cc1ccccc1-c1c[nH]c2nc(NC(=O)C3CC3)ccc12 ; O=C(Nc1ccc2c(-c3cccnc3)c[nH]c2n1)C1CC1 ; O=C(Nc1ccc2c(-c3ccncc3)c[nH]c2n1)C1CC1 ; O=C(Nc1ccc2c(-c3ccccc3O)c[nH]c2n1)C1CC1 ; O=C(Nc1ccc2c(-c3cccc(O)c3)c[nH]c2n1)C1CC1 ; COc1ncccc1-c1c[nH]c2nc(NC(=O)C3CC3)ccc12]

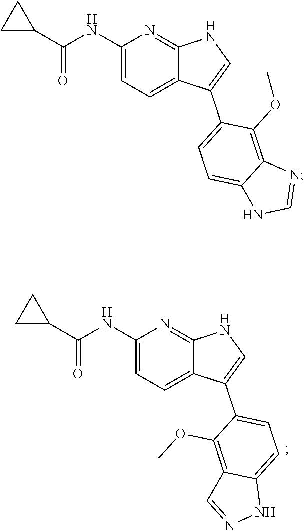 [SMILES: COc1c(-c2c[nH]c3nc(NC(=O)C4CC4)ccc23)ccc2[nH]cnc12 ; COc1c(-c2c[nH]c3nc(NC(=O)C4CC4)ccc23)ccc2[nH]ncc12]

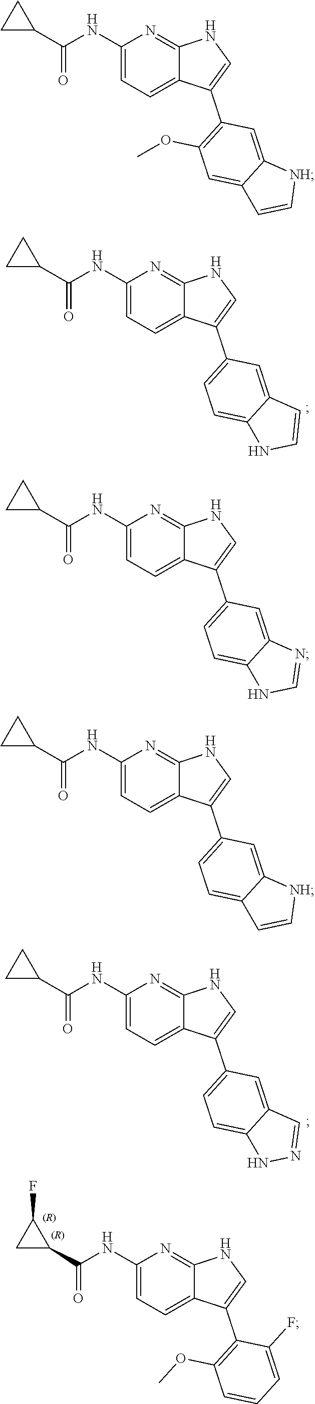

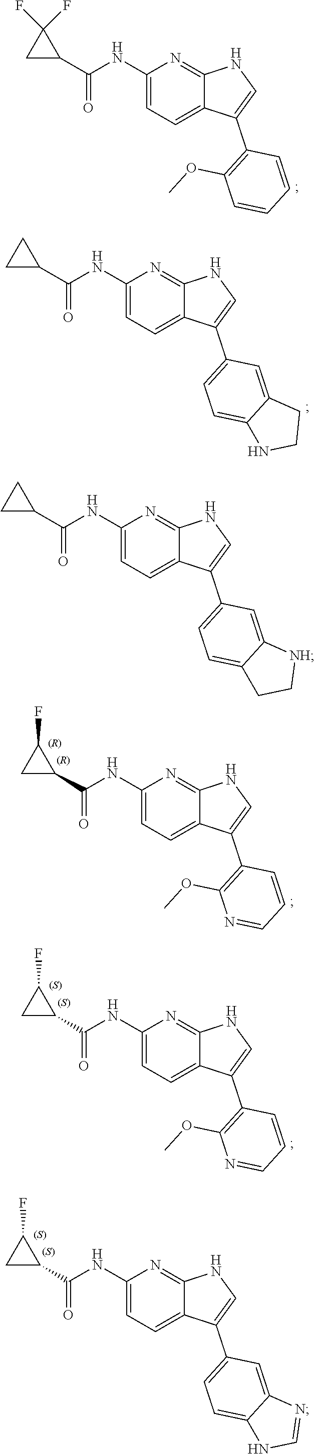

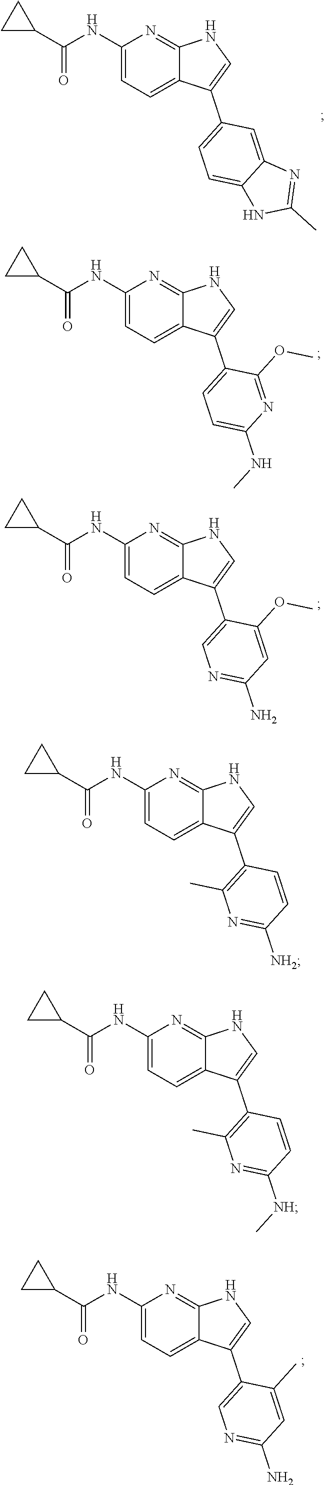 [SMILES: Cc1nc2cc(-c3c[nH]c4nc(NC(=O)C5CC5)ccc34)ccc2[nH]1 ; CNc1ccc(-c2c[nH]c3nc(NC(=O)C4CC4)ccc23)c(OC)n1 ; COc1cc(N)ncc1-c1c[nH]c2nc(NC(=O)C3CC3)ccc12 ; Cc1nc(N)ccc1-c1c[nH]c2nc(NC(=O)C3CC3)ccc12 ; CNc1ccc(-c2c[nH]c3nc(NC(=O)C4CC4)ccc23)c(C)n1 ; Cc1cc(N)ncc1-c1c[nH]c2nc(NC(=O)C3CC3)ccc12]

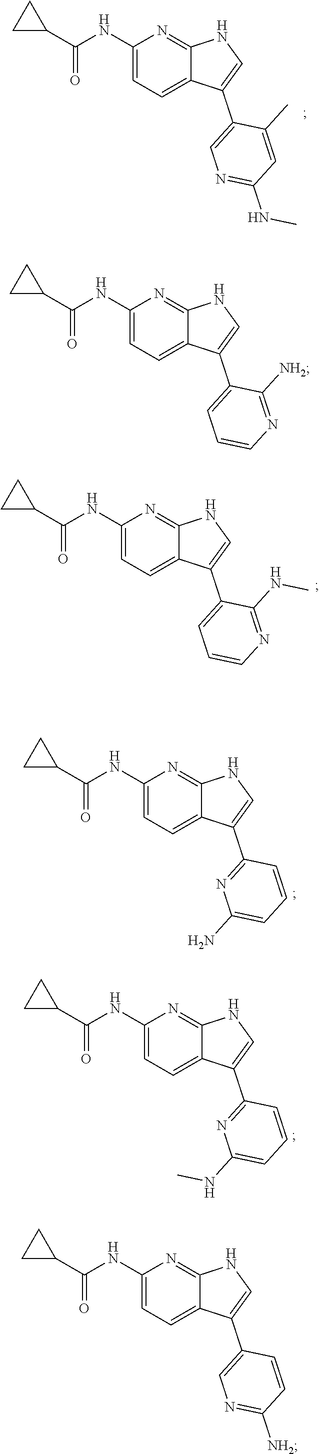 [SMILES: CNc1cc(C)c(-c2c[nH]c3nc(NC(=O)C4CC4)ccc23)cn1 ; Nc1ncccc1-c1c[nH]c2nc(NC(=O)C3CC3)ccc12 ; CNc1ncccc1-c1c[nH]c2nc(NC(=O)C3CC3)ccc12 ; Nc1cccc(-c2c[nH]c3nc(NC(=O)C4CC4)ccc23)n1 ; CNc1cccc(-c2c[nH]c3nc(NC(=O)C4CC4)ccc23)n1 ; Nc1ccc(-c2c[nH]c3nc(NC(=O)C4CC4)ccc23)cn1]

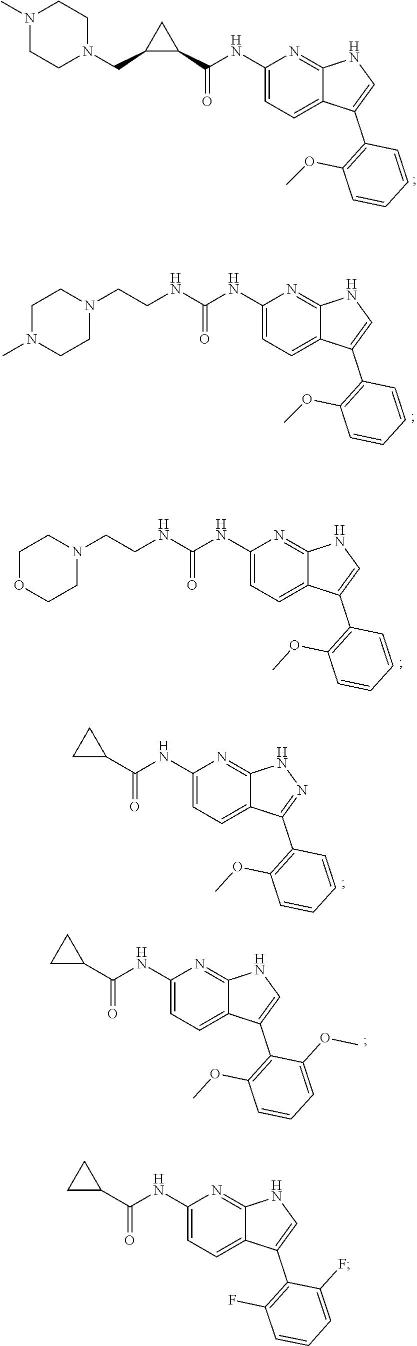 [SMILES: COc1ccccc1-c1c[nH]c2nc(NC(=O)[C@@H]3C[C@@H]3CN3CCN(C)CC3)ccc12 ; COc1ccccc1-c1c[nH]c2nc(NC(=O)NCCN3CCN(C)CC3)ccc12 ; COc1ccccc1-c1c[nH]c2nc(NC(=O)NCCN3CCOCC3)ccc12 ; COc1ccccc1-c1n[nH]c2nc(NC(=O)C3CC3)ccc12 ; COc1cccc(OC)c1-c1c[nH]c2nc(NC(=O)C3CC3)ccc12 ; O=C(Nc1ccc2c(-c3c(F)cccc3F)c[nH]c2n1)C1CC1]

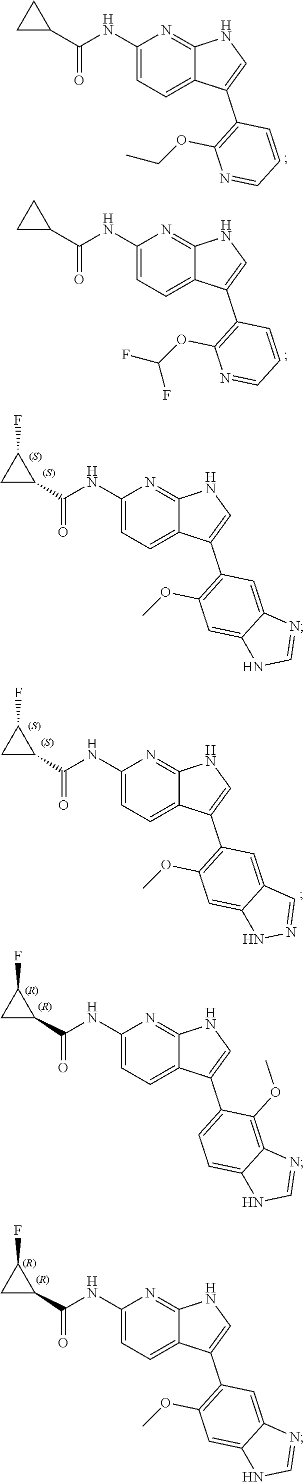

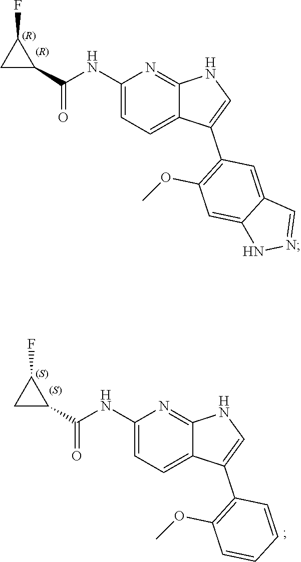

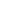

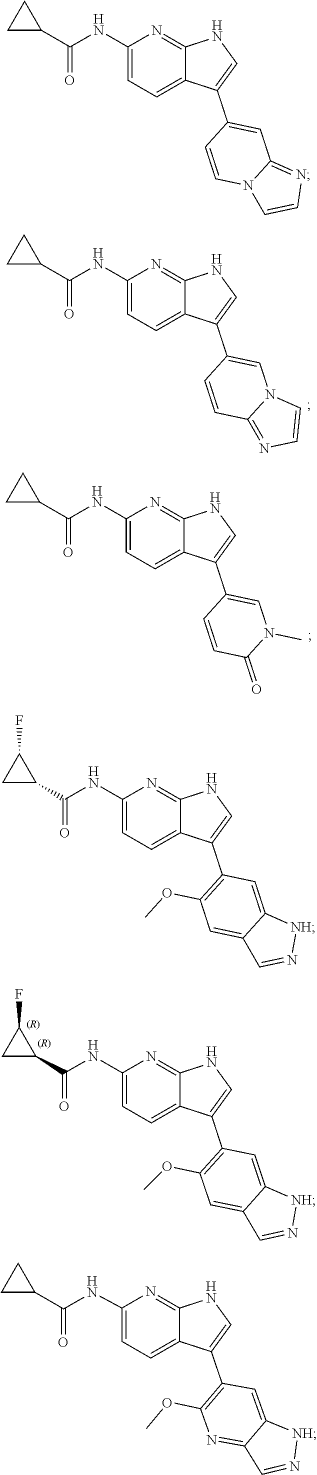

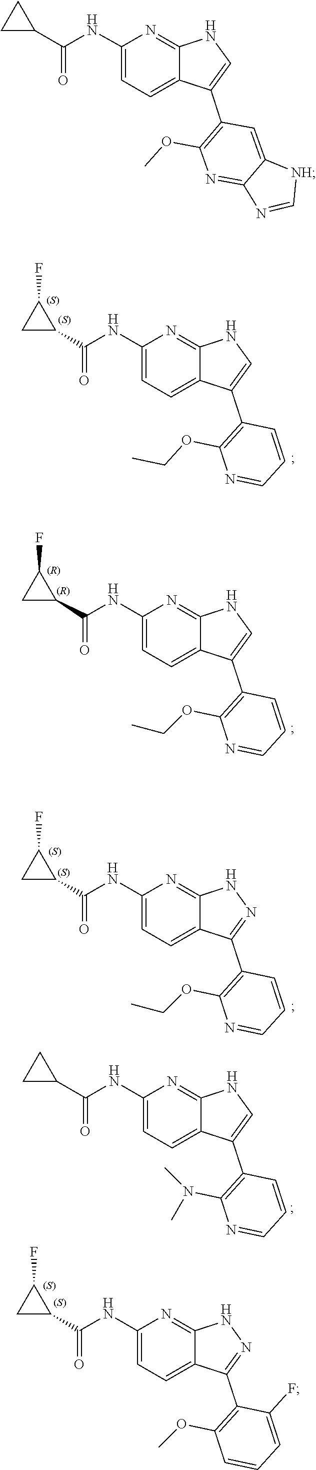

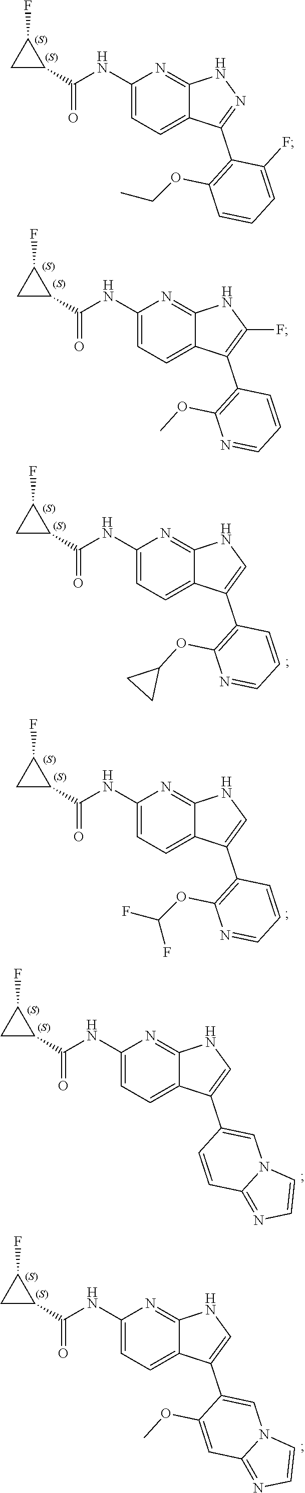

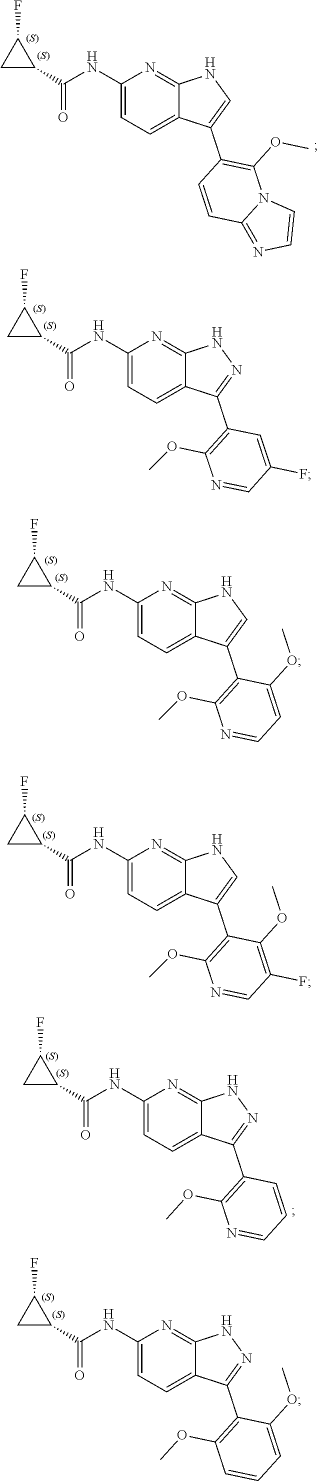

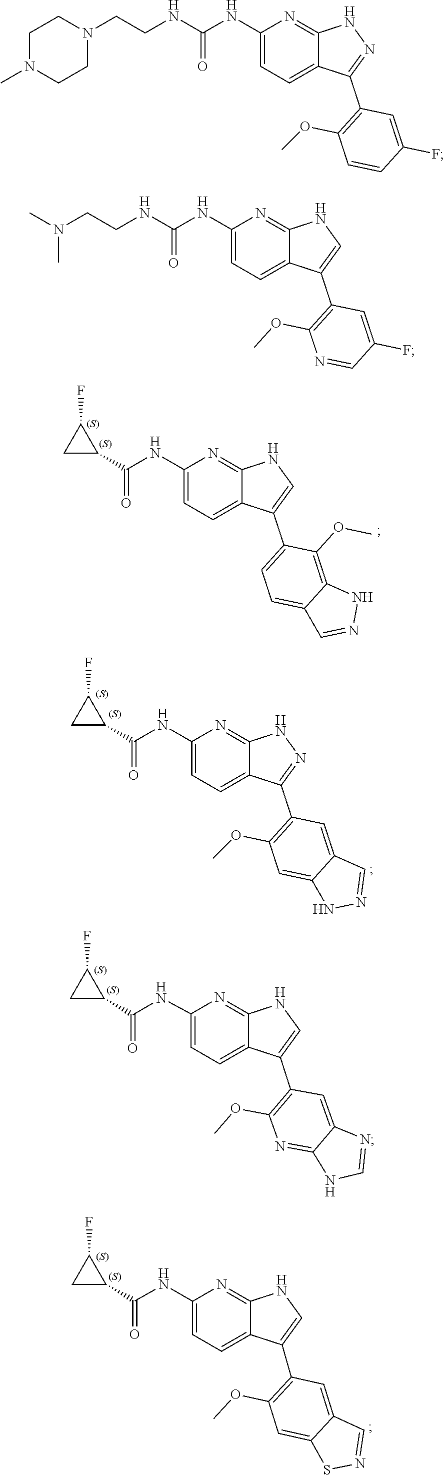

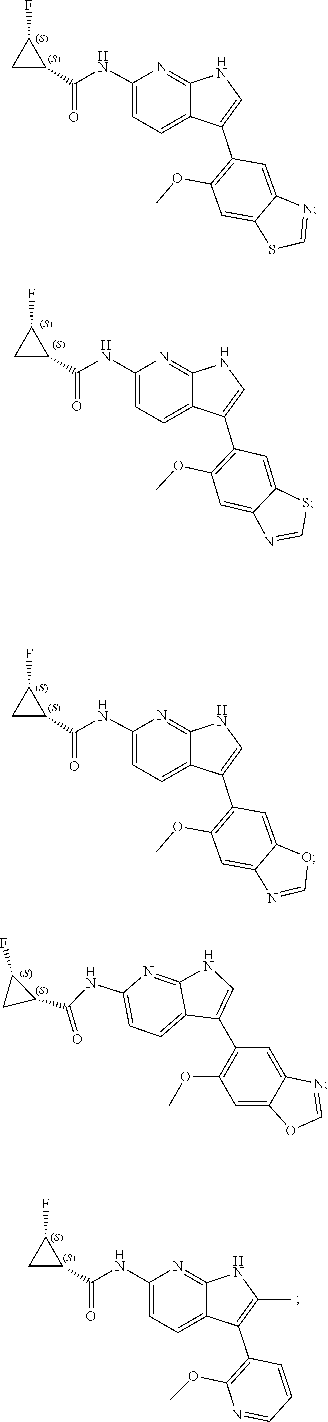

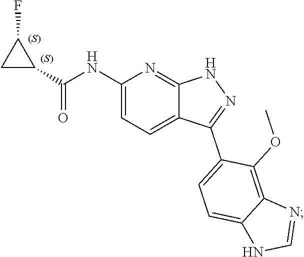

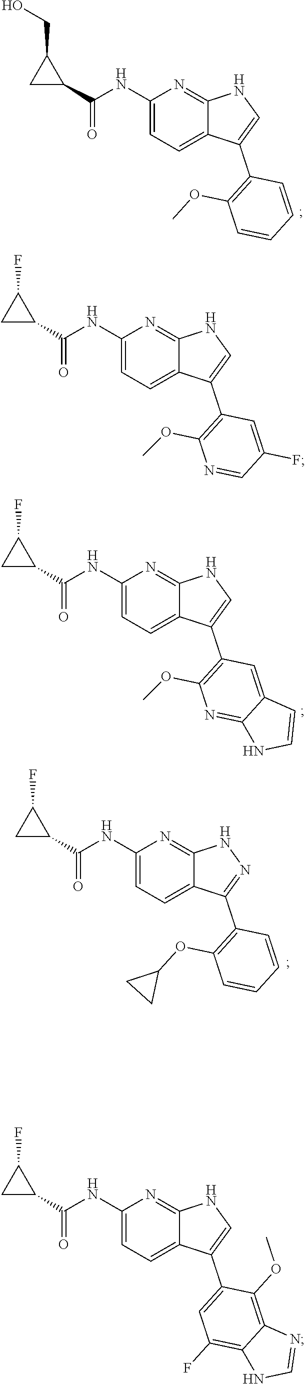 [SMILES: COc1ccccc1-c1c[nH]c2nc(NC(=O)[C@H]3C[C@H]3CO)ccc12 ; COc1ncc(F)cc1-c1c[nH]c2nc(NC(=O)[C@@H]3C[C@@H]3F)ccc12 ; COc1nc2[nH]ccc2cc1-c1c[nH]c2nc(NC(=O)[C@@H]3C[C@@H]3F)ccc12 ; O=C(Nc1ccc2c(-c3ccccc3OC3CC3)n[nH]c2n1)[C@@H]1C[C@@H]1F ; COc1c(-c2c[nH]c3nc(NC(=O)[C@@H]4C[C@@H]4F)ccc23)cc(F)c2[nH]cnc12]

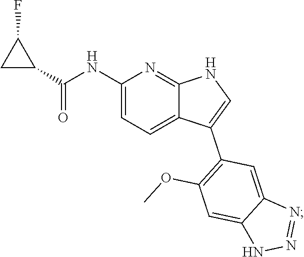 [SMILES: COc1cc2[nH]nnc2cc1-c1c[nH]c2nc(NC(=O)[C@@H]3C[C@@H]3F)ccc12]

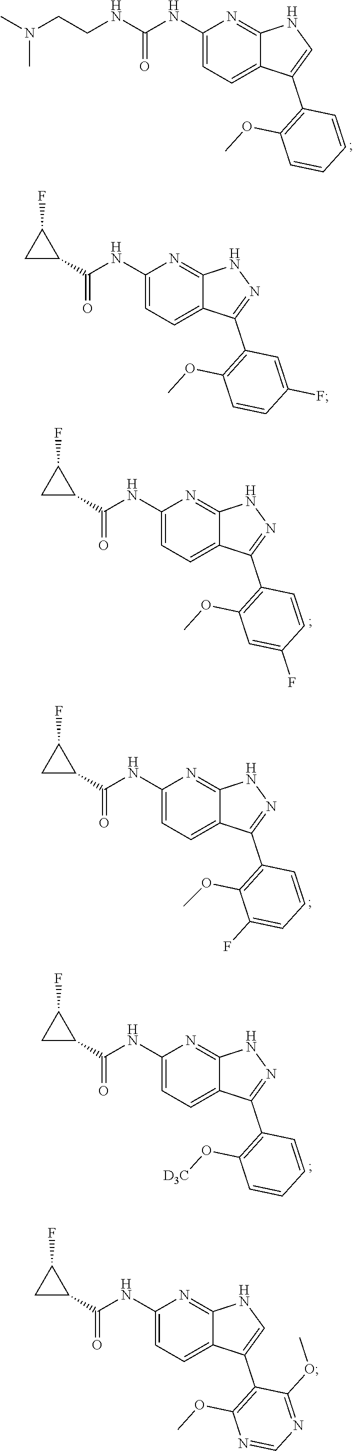 [SMILES: COc1ccccc1-c1c[nH]c2nc(NC(=O)NCCN(C)C)ccc12 ; COc1ccc(F)cc1-c1n[nH]c2nc(NC(=O)[C@@H]3C[C@@H]3F)ccc12 ; COc1cc(F)ccc1-c1n[nH]c2nc(NC(=O)[C@@H]3C[C@@H]3F)ccc12 ; COc1c(F)cccc1-c1n[nH]c2nc(NC(=O)[C@@H]3C[C@@H]3F)ccc12 ; [2H]C([2H])([2H])Oc1ccccc1-c1n[nH]c2nc(NC(=O)[C@@H]3C[C@@H]3F)ccc12 ; COc1ncnc(OC)c1-c1c[nH]c2nc(NC(=O)[C@@H]3C[C@@H]3F)ccc12]

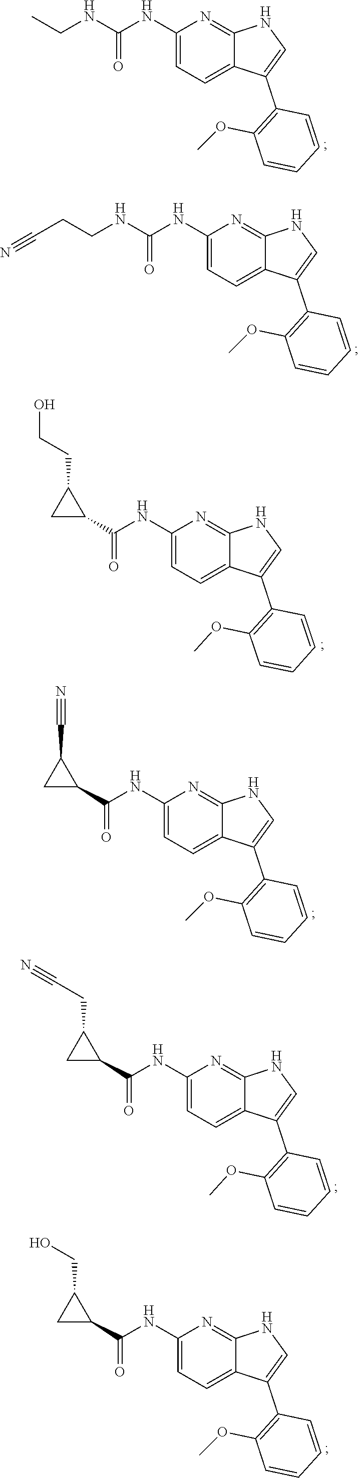 [SMILES: CCNC(=O)Nc1ccc2c(-c3ccccc3OC)c[nH]c2n1 ; COc1ccccc1-c1c[nH]c2nc(NC(=O)NCCC#N)ccc12 ; COc1ccccc1-c1c[nH]c2nc(NC(=O)[C@@H]3C[C@@H]3CCO)ccc12 ; COc1ccccc1-c1c[nH]c2nc(NC(=O)[C@H]3C[C@H]3C#N)ccc12 ; COc1ccccc1-c1c[nH]c2nc(NC(=O)[C@H]3C[C@@H]3CC#N)ccc12 ; COc1ccccc1-c1c[nH]c2nc(NC(=O)[C@H]3C[C@@H]3CO)ccc12]

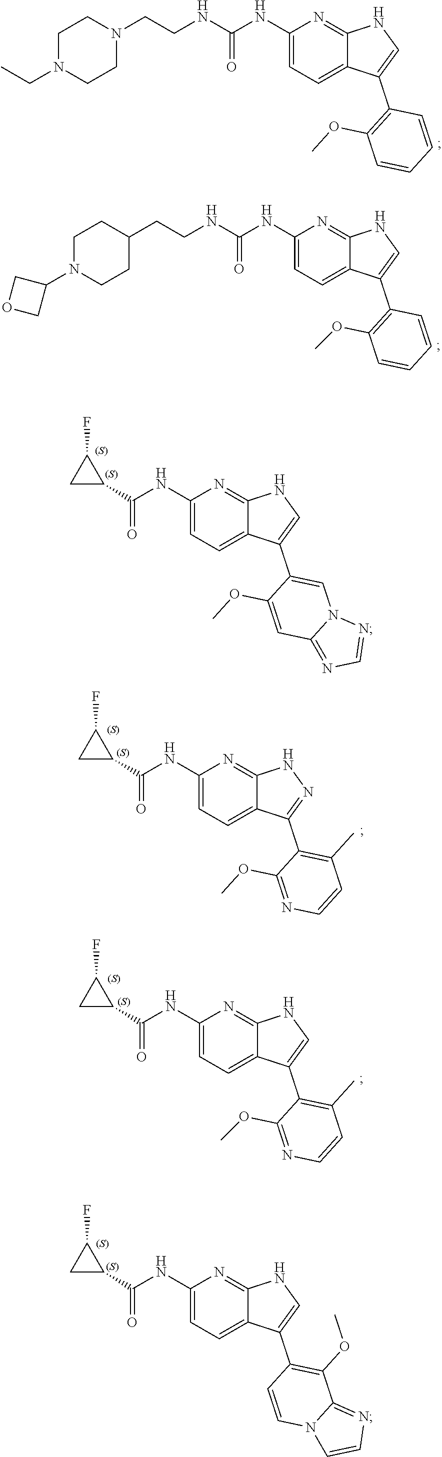

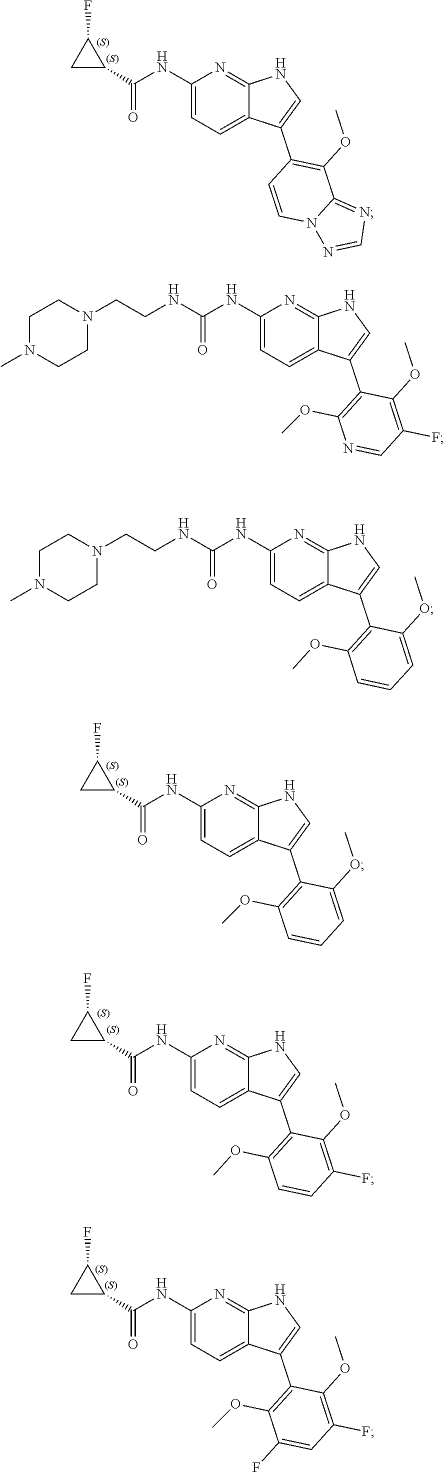

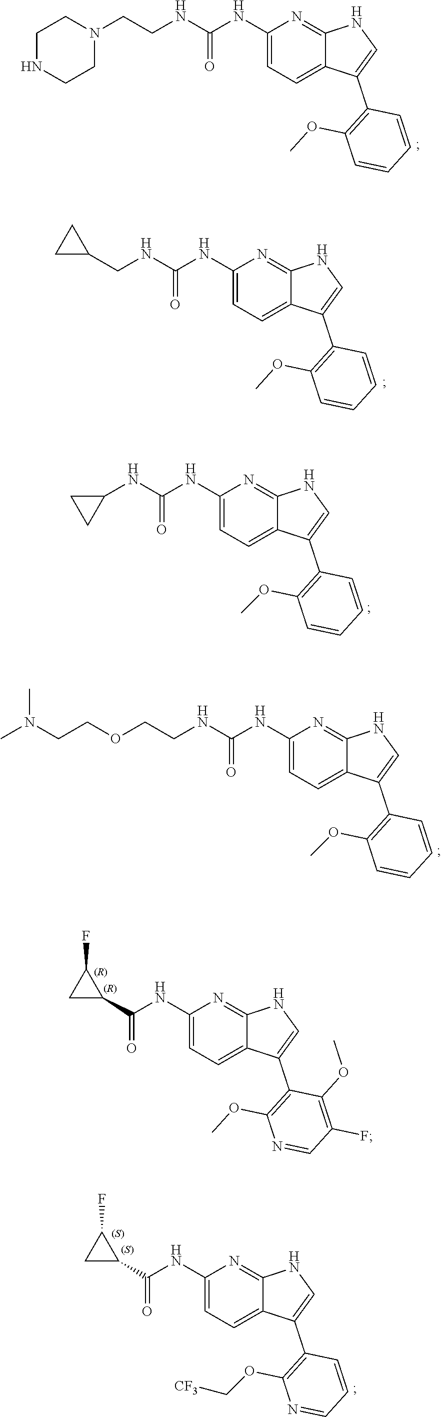

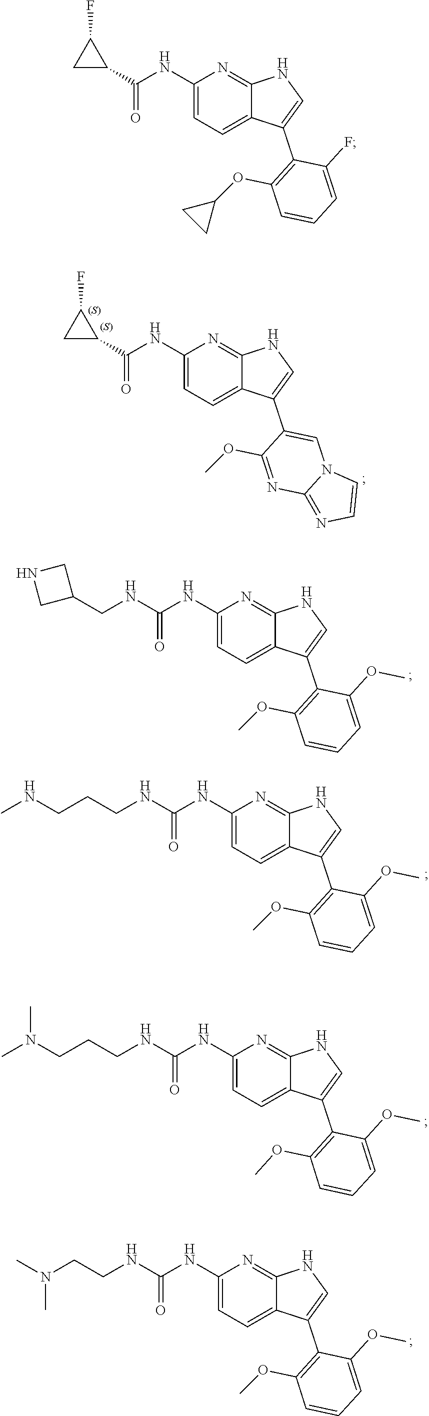

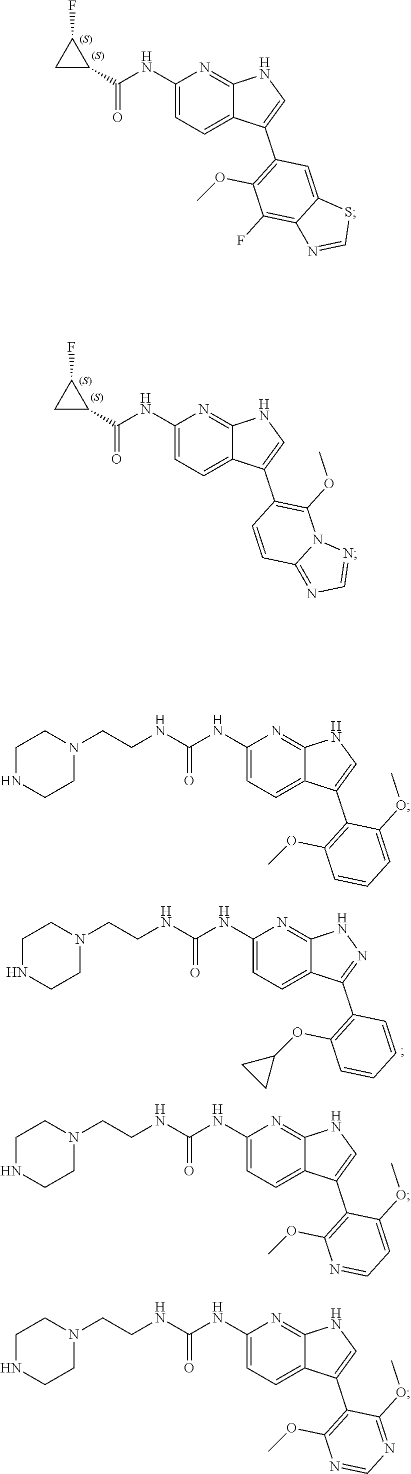

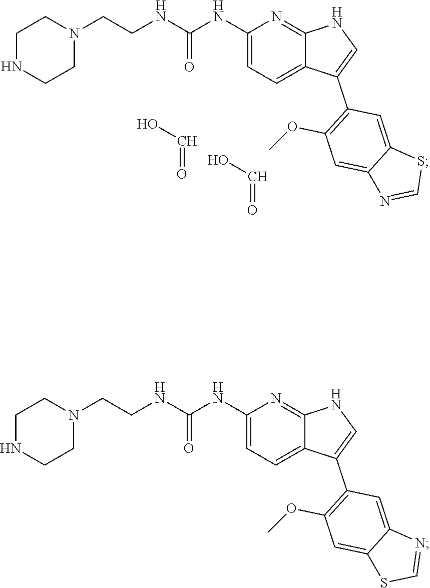

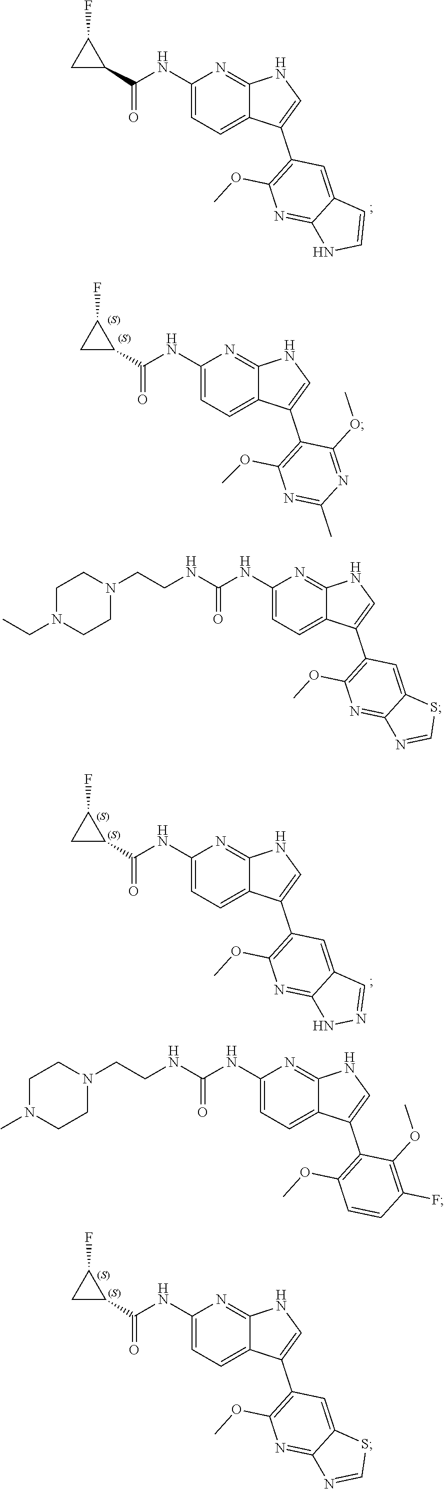

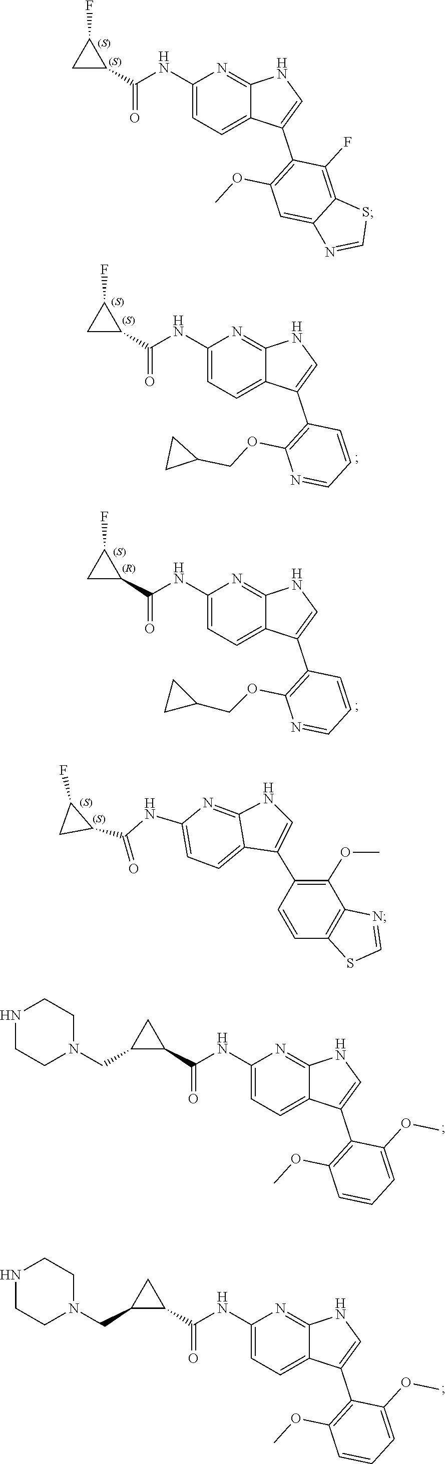

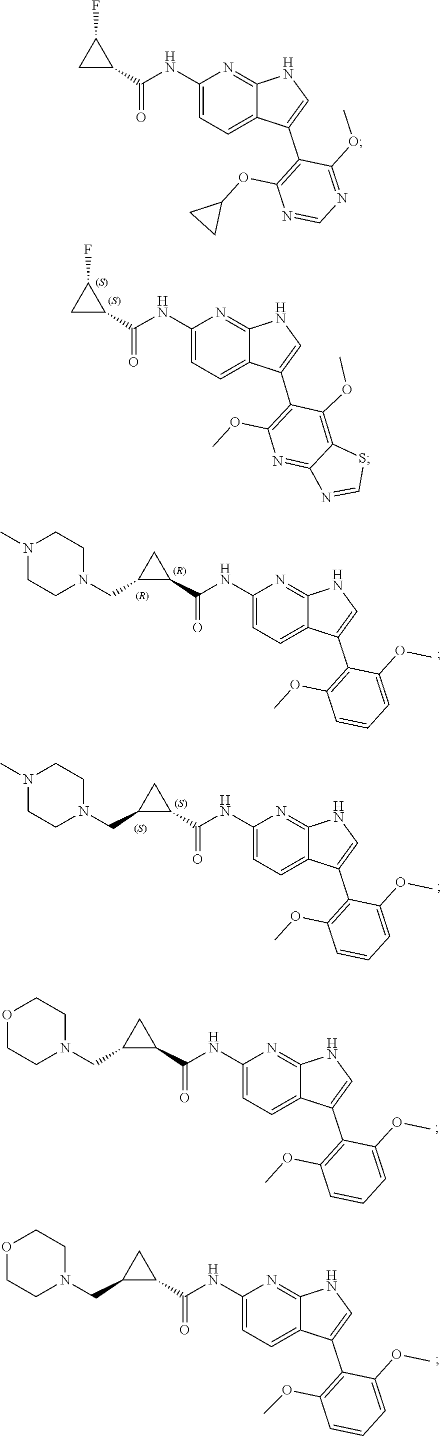

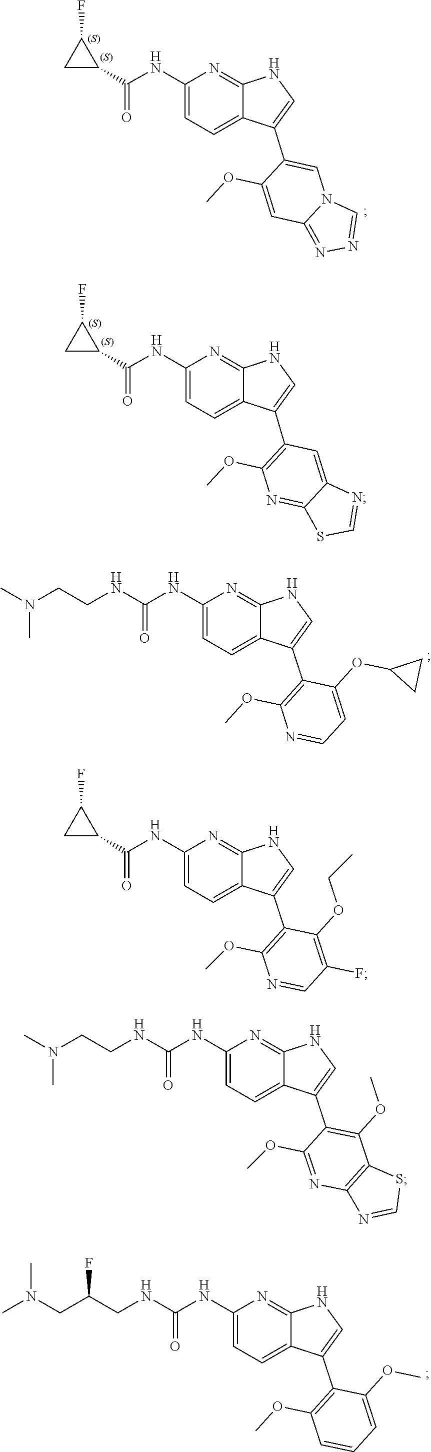

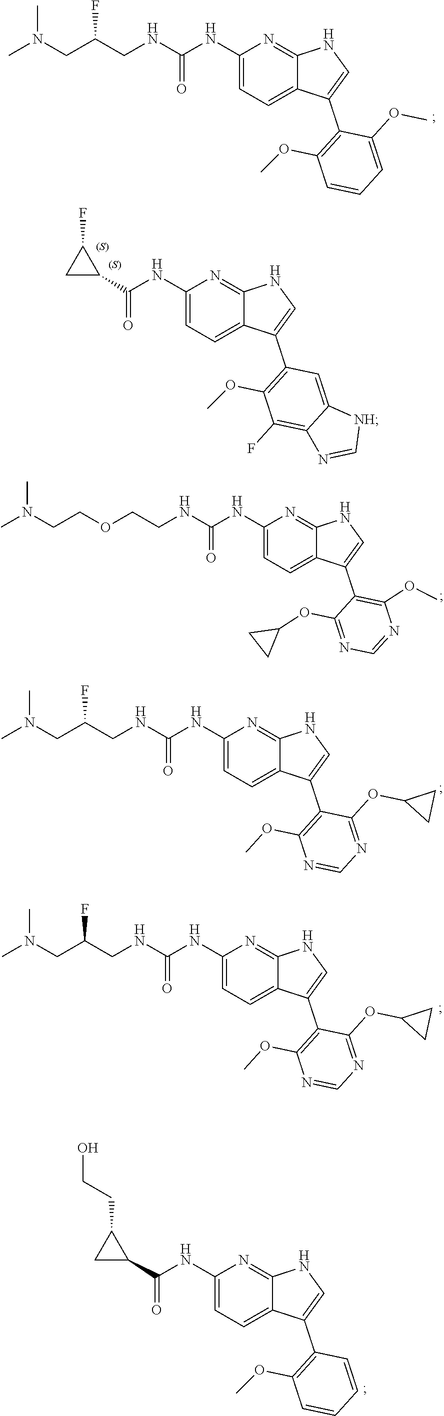

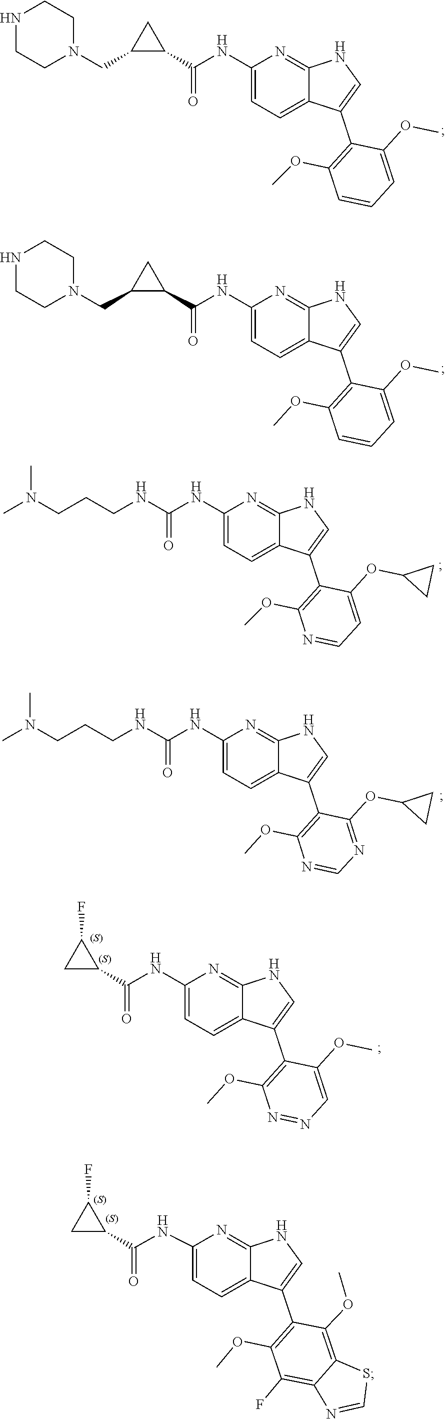

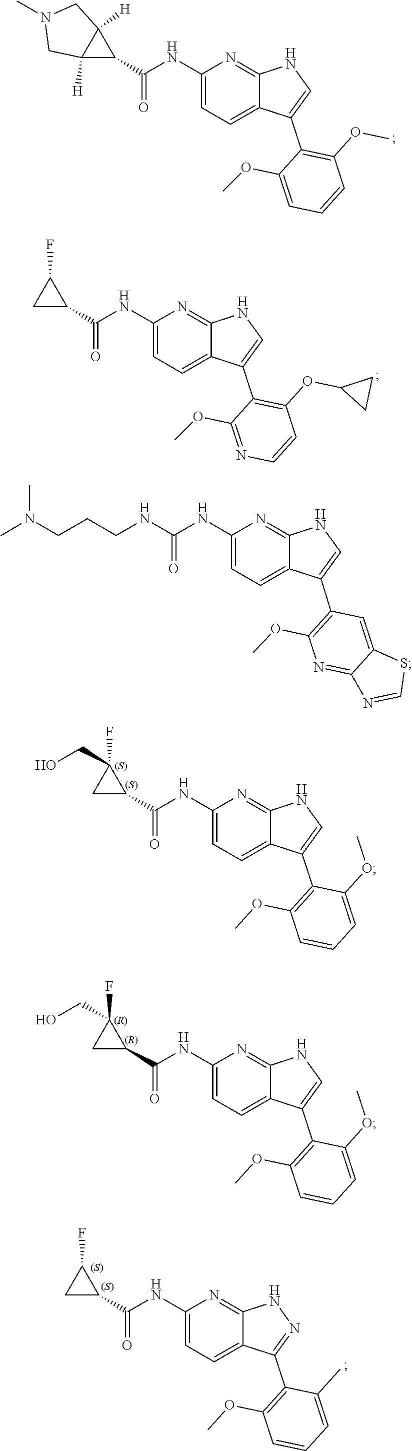

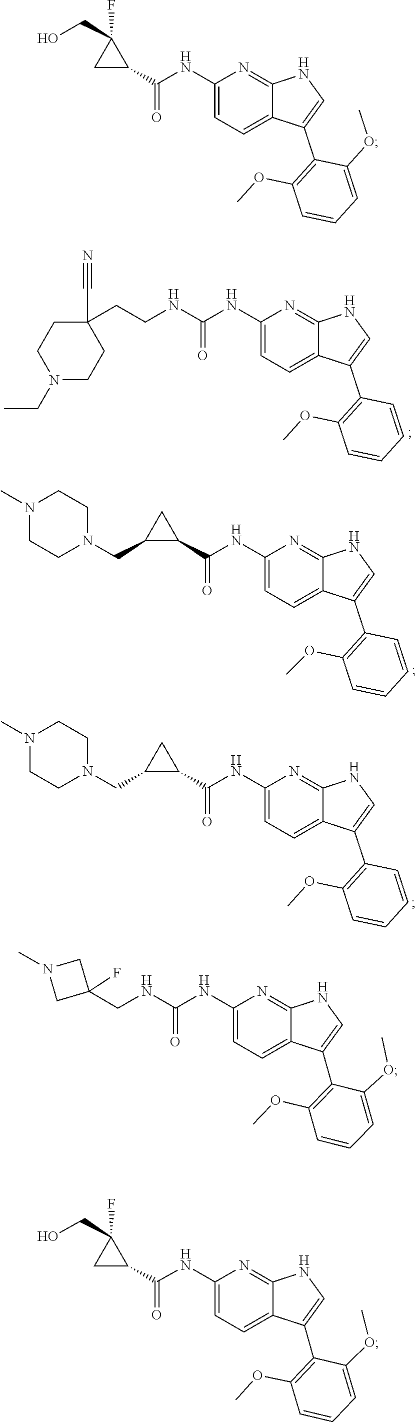 [SMILES: COc1cccc(OC)c1-c1c[nH]c2nc(NC(=O)[C@@H]3C[C@@]3(F)CO)ccc12 ; CCN1CCC(C#N)(CCNC(=O)Nc2ccc3c(-c4ccccc4OC)c[nH]c3n2)CC1 ; COc1ccccc1-c1c[nH]c2nc(NC(=O)[C@@H]3C[C@@H]3CN3CCN(C)CC3)ccc12 ; COc1ccccc1-c1c[nH]c2nc(NC(=O)[C@H]3C[C@H]3CN3CCN(C)CC3)ccc12 ; COc1cccc(OC)c1-c1c[nH]c2nc(NC(=O)NCC3(F)CN(C)C3)ccc12 ; COc1cccc(OC)c1-c1c[nH]c2nc(NC(=O)[C@@H]3C[C@@]3(F)CO)ccc12]

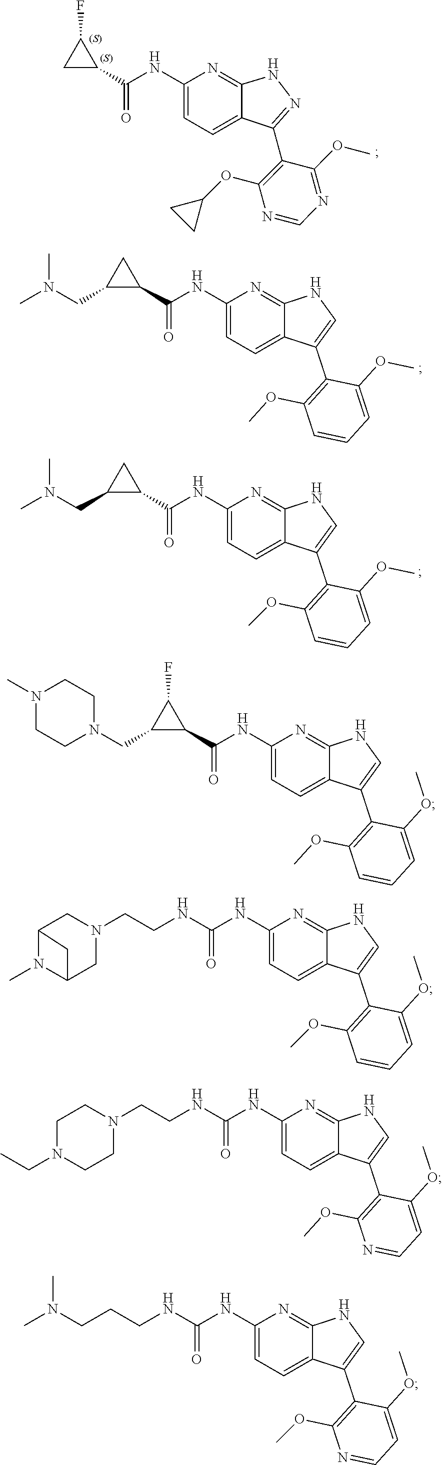

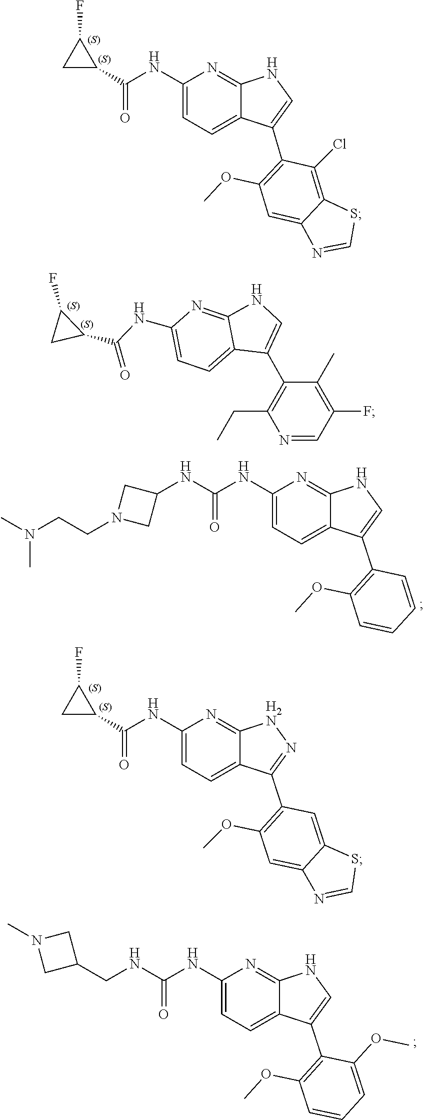

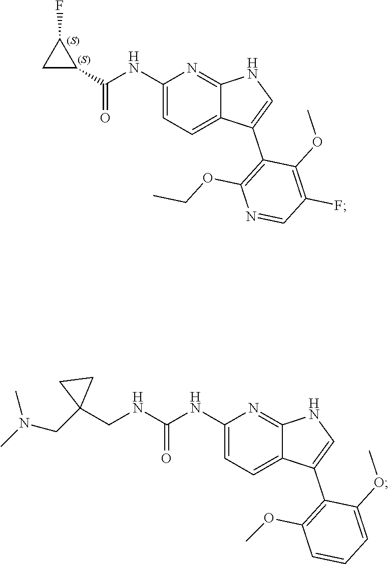

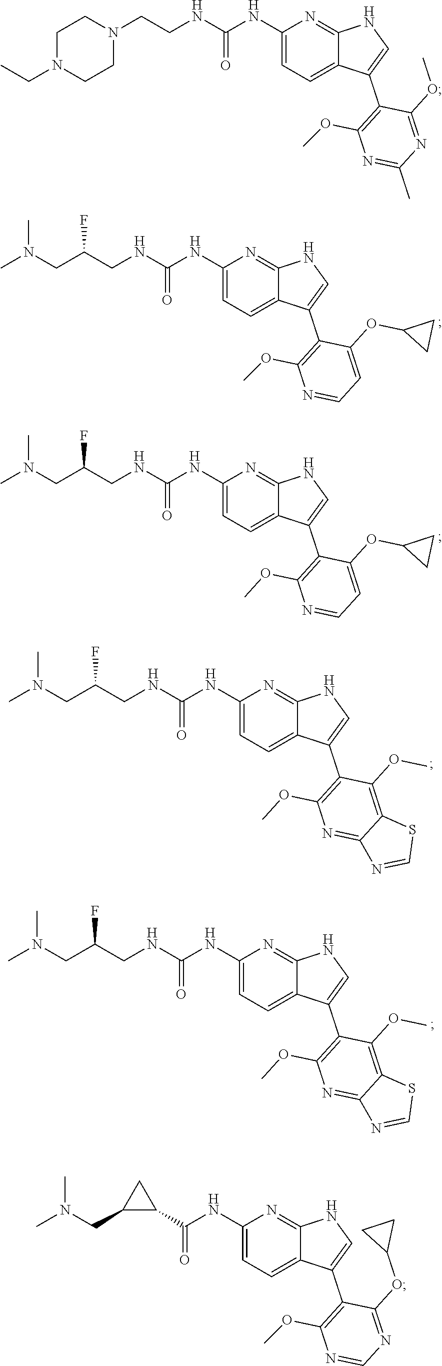

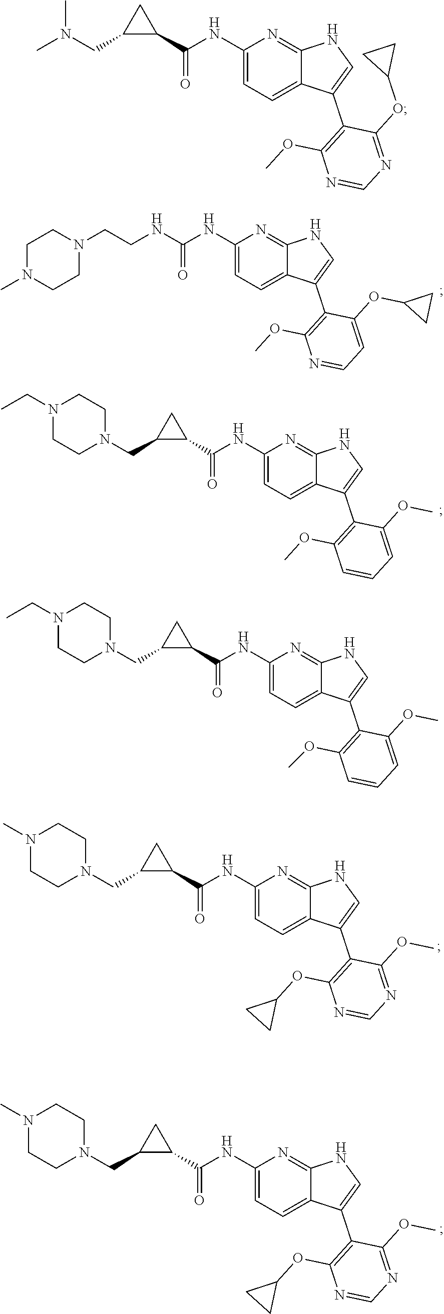

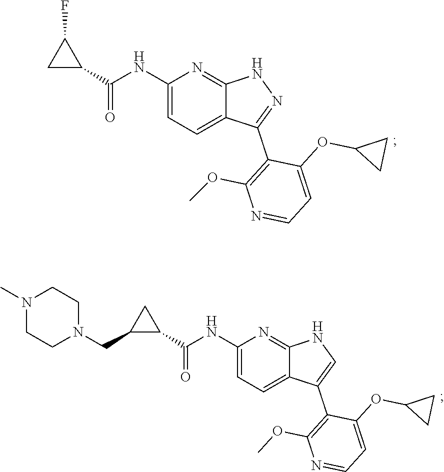 [SMILES: COc1nccc(OC2CC2)c1-c1n[nH]c2nc(NC(=O)[C@@H]3C[C@@H]3F)ccc12 ; COc1nccc(OC2CC2)c1-c1c[nH]c2nc(NC(=O)[C@H]3C[C@@H]3CN3CCN(C)CC3)ccc12]

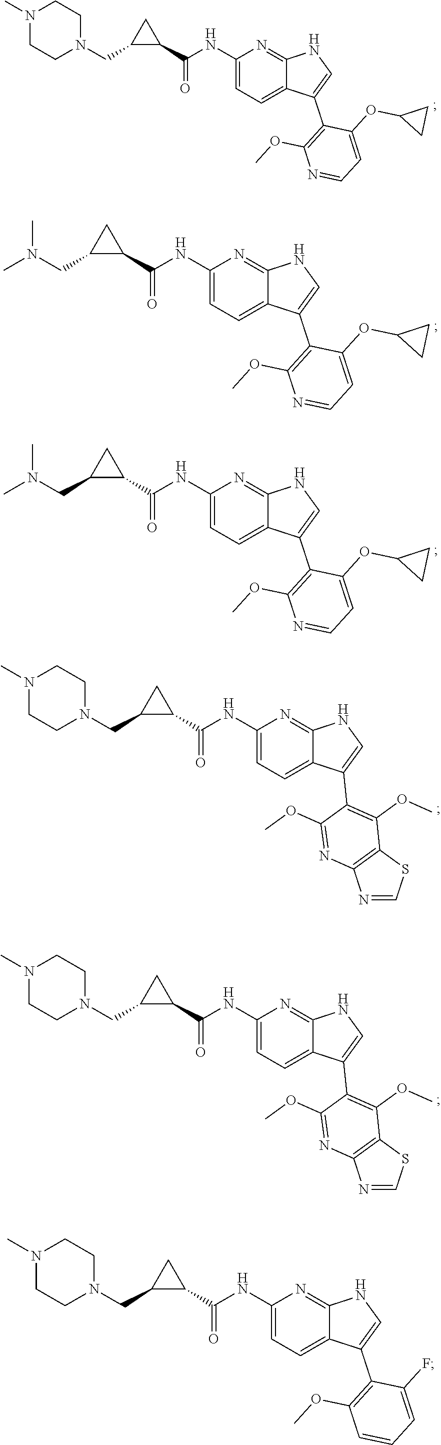

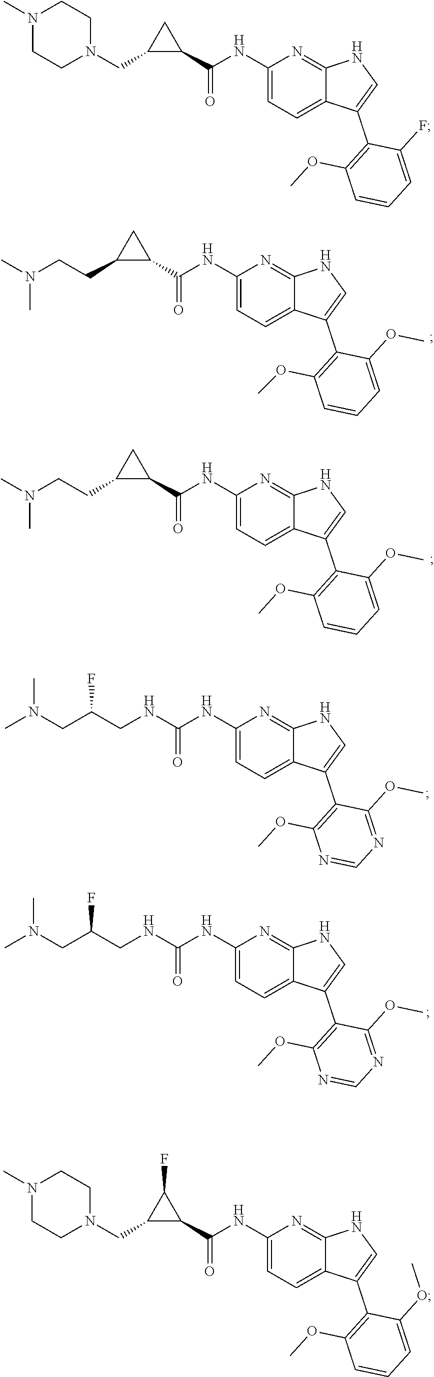

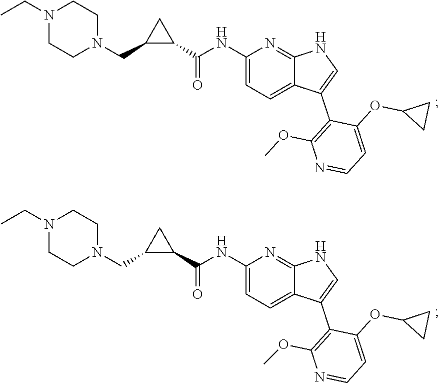 [SMILES: CCN1CCN(C[C@H]2C[C@@H]2C(=O)Nc2ccc3c(-c4c(OC5CC5)ccnc4OC)c[nH]c3n2)CC1 ; CCN1CCN(C[C@@H]2C[C@H]2C(=O)Nc2ccc3c(-c4c(OC5CC5)ccnc4OC)c[nH]c3n2)CC1]

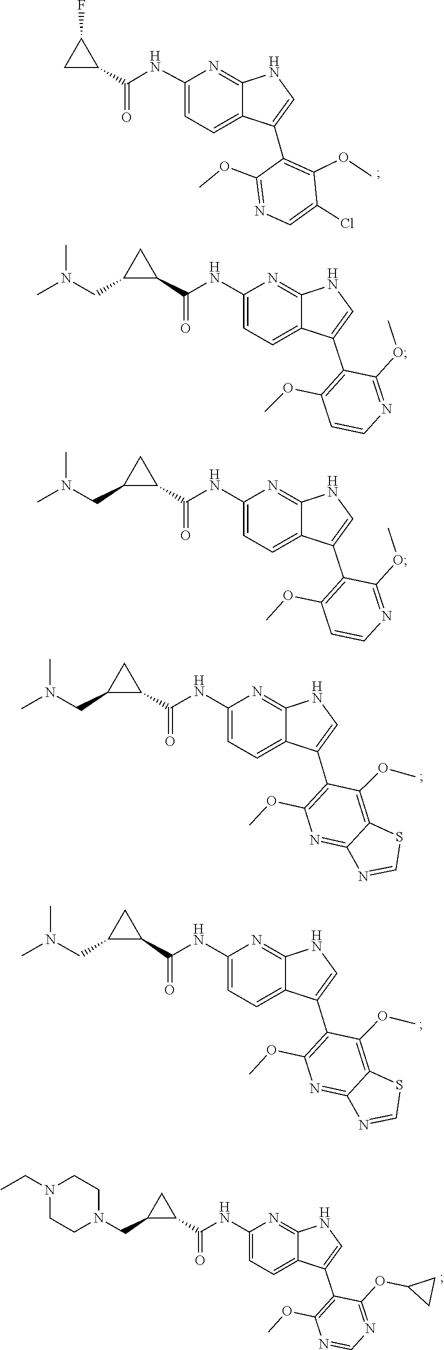

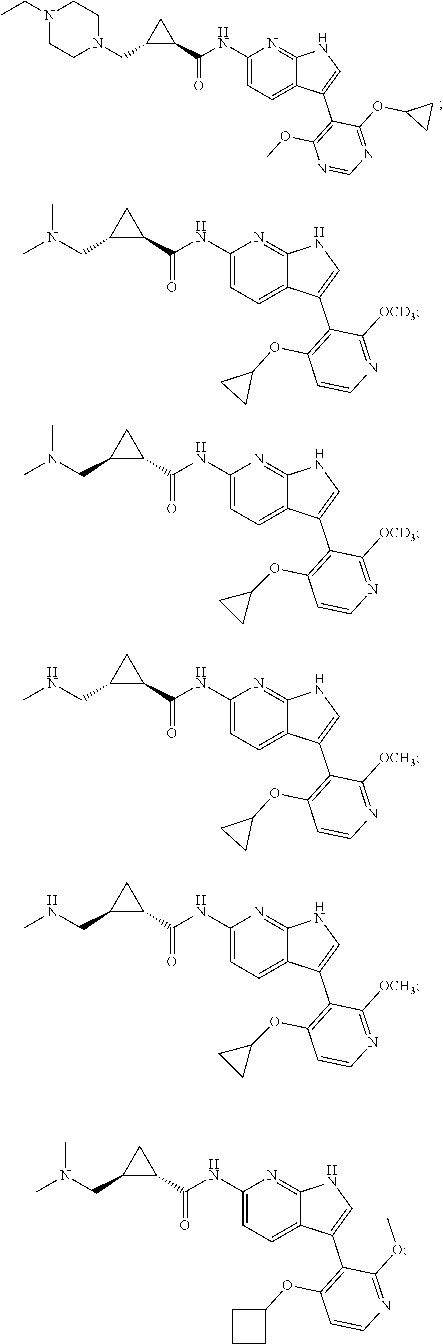

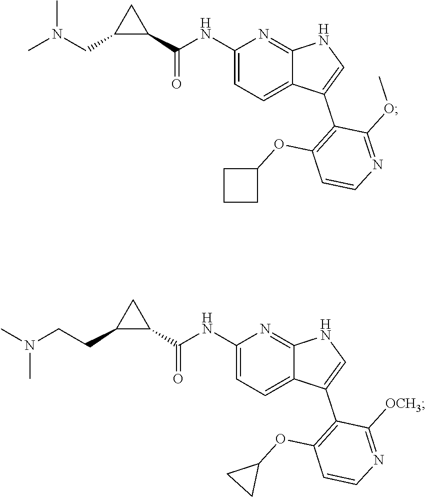 [SMILES: COc1nccc(OC2CCC2)c1-c1c[nH]c2nc(NC(=O)[C@@H]3C[C@H]3CN(C)C)ccc12 ; COc1nccc(OC2CC2)c1-c1c[nH]c2nc(NC(=O)[C@H]3C[C@@H]3CCN(C)C)ccc12]

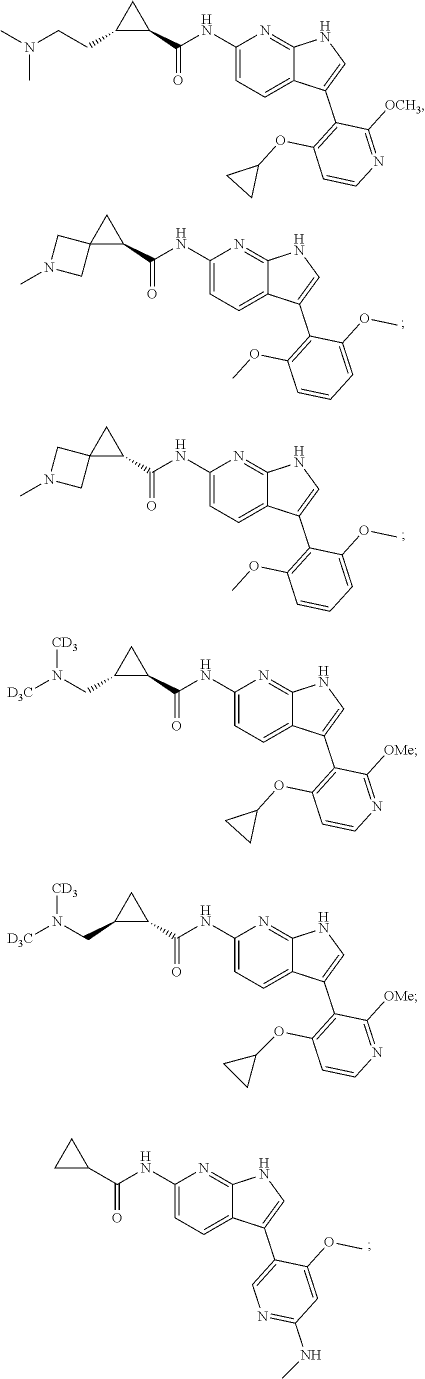

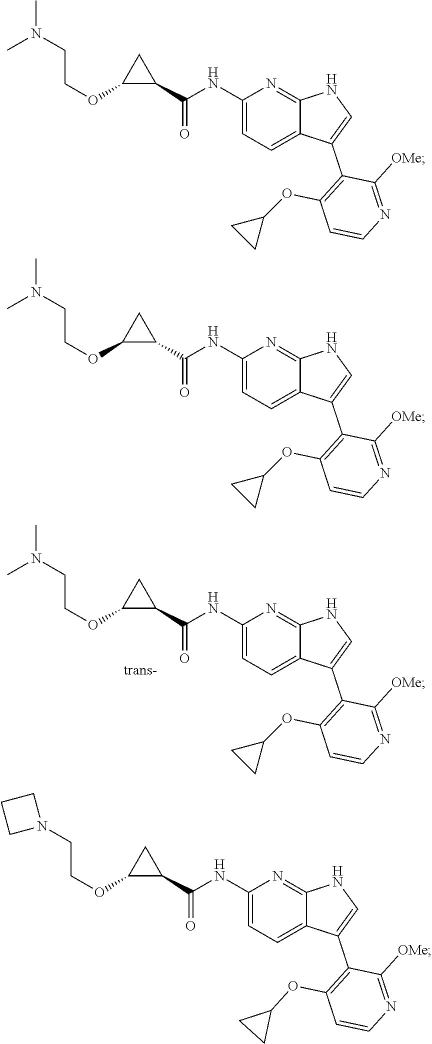 [SMILES: COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@H]3C[C@@H]3OCCN(C)C)ccc12 ; COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@@H]3C[C@H]3OCCN3CCC3)ccc12]

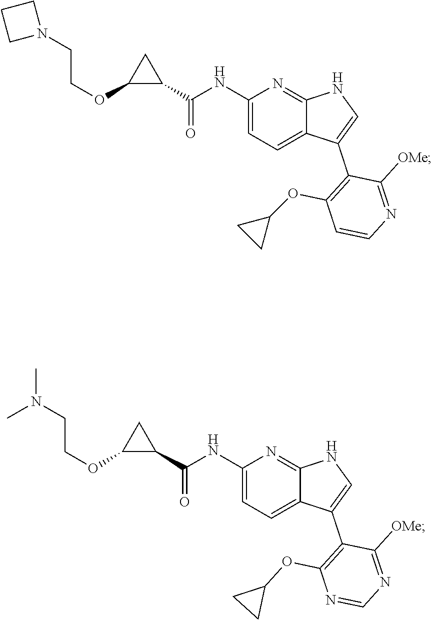 [SMILES: COc1nccc(OC2CC2)c1-c1c[nH]c2nc(NC(=O)[C@H]3C[C@@H]3OCCN3CCC3)ccc12 ; COc1ncnc(OC2CC2)c1-c1c[nH]c2nc(NC(=O)[C@@H]3C[C@H]3OCCN(C)C)ccc12]

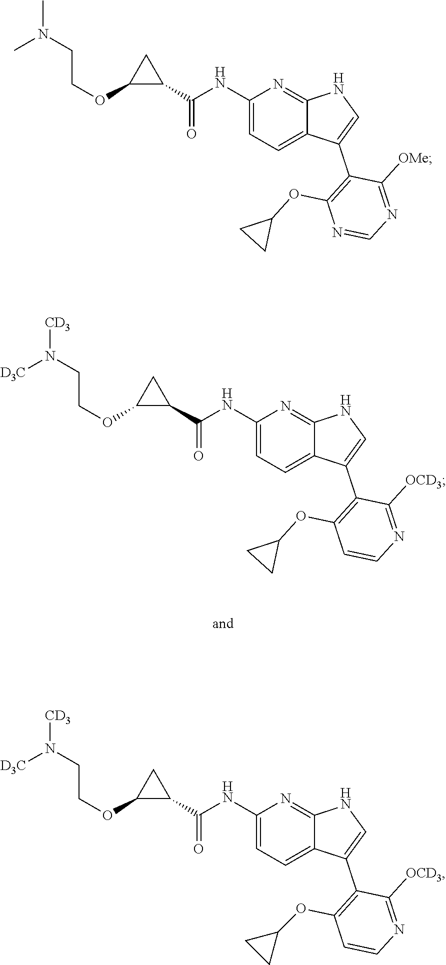

or a pharmaceutically acceptable salt, solvate, hydrate, or co-crystal thereof, or a mixture of any of the foregoing.

In another aspect, provided herein is a pharmaceutical composition comprising a compound of formula (I) as described herein, or a pharmaceutically acceptable salt, solvate, hydrate, or co-crystal thereof, or a mixture of any of the foregoing, and one or more pharmaceutically acceptable excipients.

In yet another aspect, the present disclosure provides a method of inhibiting Bcr-Abl enzymatic activity in a cell, comprising exposing the cell with an effective amount of a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

In still yet another aspect, provided herein is a method of treating chronic myeloid leukemia (CML), acute myeloid leukemia (AML), acute lymphoblastic leukemia (ALL), or a mixed phenotype acute leukemia, in a human in need thereof, comprising administering to the human a compound of formula (I) as described herein, or a pharmaceutically acceptable salt, solvate, hydrate, or co-crystal thereof, or a mixture of any of the foregoing, or a pharmaceutical composition comprising a compound of formula (I) as described herein, or a pharmaceutically acceptable salt, solvate, hydrate, or co-crystal thereof, or a mixture of any of the foregoing.

In some embodiments of the present aspect, the leukemia is refractory leukemia. In certain embodiments of the foregoing, the refractory leukemia is associated with a mutation in the Bcr-Abl tyrosine kinase gene resulting in specific amino acid substitutions selected from the group consisting of M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I. In still further embodiments of the foregoing, the refractory leukemia is associated with a mutation in the Bcr-Abl tyrosine kinase gene resulting in specific amino acid substitution T315I. In yet other embodiments, which may be combined with any preceding embodiments of the present aspect, the method further comprises administering one or more pharmaceutical agents including anti-microtubular therapies, topoisomerase inhibitors, alkylating agents, nucleotide synthesis inhibitors, DNA synthesis inhibitors, protein synthesis inhibitors, developmental signaling pathway inhibitors, pro-apoptotic agents, Abl myristoyl-pocket binding inhibitors, MEK1/2 inhibitors, AKT inhibitors, PI3K inhibitors and/or radiation.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### I. Definitions

As used herein, the following definitions shall apply unless otherwise indicated. Further, if any term or symbol used herein is not defined as set forth below, it shall have its ordinary meaning in the art.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual”, “subject” and “patient” refer to mammals and includes humans and non-human mammals. Examples of patients include, but are not limited to, mice, rats, hamsters, guinea pigs, pigs, rabbits, cats, dogs, goats, sheep, cows, and humans. In some embodiments, patient refers to a human.

As used herein, the term “mammal” includes, but is not limited to, humans, mice, rats, guinea pigs, monkeys, dogs, cats, horses, cows, pigs, and sheep.

“Pharmaceutically acceptable” refers to safe and non-toxic, and suitable for in vivo or for human administration.

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

The term “cycloalkyl,” “carbocyclic,” or “carbocycle” refers to hydrocarbon rings having the indicated number of ring atoms (e.g., C₃-C₆ cycloalkyl means 3-6 carbons) and being fully saturated or having no more than one double bond between ring vertices. As used herein, “cycloalkyl,” “carbocyclic,” or “carbocycle” is also meant to refer to bicyclic, polycyclic and spirocyclic hydrocarbon rings such as, for example, bicyclo[2.2.1]heptane, pinane, bicyclo[2.2.2]octane, adamantane, norborene, spirocyclic C₅₋₁₂ alkane, etc. In some embodiments, “cycloalkyl” encompasses C₃-C₇ cycloalkyl, C₄-C₇ cycloalkyl, C₅-C₇ cycloalkyl, C₅-C₇ cycloalkyl, C₃-C₆ cycloalkyl, C₄-C₆ cycloalkyl, C₅-C₆ cycloalkyl, C₃-C₅ cycloalkyl, C₄-C₅ cycloalkyl, or C₃-C₄ cycloalkyl. In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group.

The term “heteroalkyl,” by itself or in combination with another term, means, unless otherwise stated, a stable straight or branched chain hydrocarbon radical, consisting of the stated number of carbon atoms and from one to three heteroatoms selected from the group consisting of O, N, Si and S, and wherein the nitrogen and sulfur atoms can optionally be oxidized and the nitrogen heteroatom can optionally be quaternized. The heteroatom(s) 0, N and S can be placed at any interior position of the heteroalkyl group. The heteroatom Si can be placed at any position of the heteroalkyl group, including the position at which the alkyl group is attached to the remainder of the molecule. A “heteroalkyl” can contain up to three units of unsaturation, and also include mono- and poly-halogenated variants, or combinations thereof. Examples include —CH₂—CH₂—O—CH₃, —CH₂—CH₂—O—CF₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —Si(CH₃)₃, —CH₂—CH═N—OCH₃, and —CH═CH═N(CH₃)—CH₃. Up to two heteroatoms can be consecutive, such as, for example, —CH₂—NH—OCH₃ and —CH₂—O—Si(CH₃)₃.

The term “heterocycloalkyl,” “heterocyclic,” or “heterocycle” refers to a cycloalkyl radical group having the indicated number of ring atoms (e.g., 5-6 membered heterocycloalkyl) that contain from one to five heteroatoms selected from the group consisting of N, O, and S, wherein the nitrogen and sulfur atoms are optionally oxidized, nitrogen atom(s) are optionally quaternized, as ring atoms. Unless otherwise stated, a “heterocycloalkyl,” “heterocyclic,” or “heterocycle” ring can be a monocyclic, a bicyclic, bridged or fused ring system, spirocyclic or a polycylic ring system. Non-limiting examples of “heterocycloalkyl,” “heterocyclic,” or “heterocycle” rings include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-5-oxide, thiomorpholine-S,S-oxide, piperazine, pyran, pyridone, 3-pyrroline, thiopyran, pyrone, tetrahydrofuran, tetrhydrothiophene, quinuclidine, tropane and the like. A “heterocycloalkyl,” “heterocyclic,” or “heterocycle” group can be attached to the remainder of the molecule through one or more ring carbons or heteroatoms. In some embodiments, “heterocycloalkyl” encompasses 3- to 10-membered heterocycloalkyl, 4- to 10-membered heterocycloalkyl, 5- to 10-membered heterocycloalkyl, 6- to 10-membered heterocycloalkyl, 7- to 10-membered heterocycloalkyl, 8- to 10-membered heterocycloalkyl, 9- to 10-membered heterocycloalkyl, 3- to 9-membered heterocycloalkyl, 4- to 9-membered heterocycloalkyl, 5- to 9-membered heterocycloalkyl, 6- to 9-membered heterocycloalkyl, 7- to 9-membered heterocycloalkyl, 8- to 9-membered heterocycloalkyl, 3- to 8-membered heterocycloalkyl, 4- to 8-membered heterocycloalkyl, 5- to 8-membered heterocycloalkyl, 6- to 8-membered heterocycloalkyl, 7- to 8-membered heterocycloalkyl, 3- to 7-membered heterocycloalkyl, 4- to 7-membered heterocycloalkyl, 5- to 7-membered heterocycloalkyl, 6- to 7-membered heterocycloalkyl, 3- to 6-membered heterocycloalkyl, 4- to 6-membered heterocycloalkyl, 5- to 6-membered heterocycloalkyl, 3- to 5-membered heterocycloalkyl, 4- to 5-membered heterocycloalkyl, or 3- to 4-membered heterocycloalkyl. In other embodiments, “heterocycloalkyl” may be characterized by the number of carbon atoms in the ring, provided that the ring contains at least one heteroatom. For example, in some embodiments, “heterocycloalkyl” encompasses C₃-C₉ heterocycloalkyl, C₃-C₅ heterocycloalkyl, C₃-C₇ heterocycloalkyl, C₃-C₆ heterocycloalkyl, C₃-C₅ heterocycloalkyl, C₃-C₄ heterocycloalkyl, C₄-C₉ heterocycloalkyl, C₄-C₅ heterocycloalkyl, C₄-C₇ heterocycloalkyl, C₄-C₆ heterocycloalkyl, C₄-C₅ heterocycloalkyl, C₅-C₉ heterocycloalkyl, C₃-C₈ heterocycloalkyl, C₅-C₇ heterocycloalkyl, C₅-C₆ heterocycloalkyl, C₆-C₉ heterocycloalkyl, C₆-C₈ heterocycloalkyl, C₆-C₇ heterocycloalkyl, C₇-C₉ heterocycloalkyl, C₇-C₈ heterocycloalkyl, or C₅-C₉ heterocycloalkyl. It should be recognized that “heterocycloalkyl” as described by the number of ring atoms may also be described by number of carbon atoms in the ring. For example, a piperazinyl ring may be described as a C₄ heterocycloalkyl ring or a 6-membered heterocycloalkyl ring; an azetidinyl or oxetanyl ring may each be described as a C₃ heterocycloalkyl ring or a 4-membered heterocycloalkyl ring.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. In some embodiments, an alkyl (or alkylene) group will have 10 or fewer carbon atoms.

The term “heteroalkylene” by itself or as part of another substituent means a divalent radical, saturated or unsaturated or polyunsaturated, derived from heteroalkyl, as exemplified by —CH₂—CH₂—S—CH₂CH₂—, —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The term “heterocycloalkylene” by itself or as part of another substituent means a divalent radical, saturated or unsaturated or polyunsaturated, derived from heterocycloalkyl. For heterocycloalkylene groups, heteroatoms can also occupy either or both of the chain termini.

The terms “alkoxy” and “alkylamino” are used in their conventional sense, and refer to those alkyl groups attached to the remainder of the molecule via an oxygen atom or an amino group, respectively.

The term “heterocycloalkoxy” refers to a heterocycloalkyl-O— group in which the heterocycloalkyl group is as previously described herein.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “C₁-C₄ haloalkyl” is mean to include trifluoromethyl, 2,2,2-trifluoroethyl, 4-chlorobutyl, 3-bromopropyl, difluoromethyl, and the like.

The term “haloalkyl-OH” refers to a haloalkyl group as described above which is also substituted by one or more hydroxyl groups. The term “haloalkyl-OH” is meant to include haloalkyl substituted by one hydroxyl group, as well as haloalkyl substituted by multiple hydroxyl groups. For example, the term “haloalkyl-OH” includes —CH(F)OH, —CH₂CFHCH₂OH, —CH(OH)CF₃, and the like.

The term “alkyl-OH” refers to an alkyl substituted by one or more hydroxyl groups. The term “alkyl-OH” is meant to include alkyl substituted by one hydroxyl group, as well as alkyl substituted by multiple hydroxyl groups. For example, the term “alkyl-OH” includes —CH₂OH, —CH(OH)CH₃, —CH₂CH₂OH, —C(CH₃)₂OH, and the like.

The term “alkyl-CN” refers to an alkyl substituted by one or more cyano groups. The term “alkyl-CN” is meant to include alkyl substituted by one cyano group, as well as alkyl substituted by multiple cyano groups. For example, the term “alkyl-CN” includes —CH₂CN, —CH₂CH₂CN, —CH(CN)CH₃, and the like.

The term “aryl” means, unless otherwise stated, a polyunsaturated, typically aromatic, hydrocarbon group, which can be a single ring or multiple rings (up to three rings) which are fused together. In some embodiments, “aryl” encompasses C₆-C₁₄ aryl, C₅-C₁₄ aryl, C₁₀-C₁₄ aryl, C₁₂-C₁₄ aryl, C₆-C₁₂ aryl, C₅-C₁₂ aryl, C₁₀-C₁₂ aryl, C₆-C₁₀ aryl, C₅-C₁₀ aryl, or C₆-C₈ aryl. In some embodiments, both rings of a polycyclic aryl group are aromatic (e.g., naphthyl). In other embodiments, polycyclic aryl groups may include a non-aromatic ring fused to an aromatic ring, provided the polycyclic aryl group is bound to the parent structure via an atom in the aromatic ring. Thus, in some embodiments, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, in some embodiments, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl,” as defined herein, regardless of the point of attachment (e.g., both quinolin-5-yl and quinolin-2-yl are heteroaryl groups). In some embodiments, aryl is phenyl or naphthyl. In certain embodiments, aryl is phenyl.

The term “heteroaryl” refers to aryl groups (or rings) that contain from one to five heteroatoms selected from the group consisting of N, O, and S, wherein the nitrogen and sulfur atoms are optionally oxidized, and the nitrogen atom(s) are optionally quaternized. A heteroaryl group can be attached to the remainder of the molecule through a carbon atom or a heteroatom as valency permits. In some embodiments, both rings of a polycyclic heteroaryl group are aromatic. In other embodiments, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, in some embodiments, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group.

Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the term “heteroaryl” encompasses 5- to 10-membered heteroaryl, 6- to 10-membered heteroaryl, 7- to 10-membered heteroaryl, 8- to 10-membered heteroaryl, 9- to 10-membered heteroaryl, 5- to 9-membered heteroaryl, 6- to 9-membered heteroaryl, 7- to 9-membered heteroaryl, 8- to 9-membered heteroaryl, 5- to 8-membered heteroaryl, 6- to 8-membered heteroaryl, 7- to 8-membered heteroaryl, 5- to 7-membered heteroaryl, 6- to 7-membered heteroaryl, or 5- to 6-membered heteroaryl.

The above terms (e.g., “alkyl,” “aryl” and “heteroaryl”), in some embodiments, will include both substituted and unsubstituted forms of the indicated radical. The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocycloalkyl, heterocycloalkenyl, aralkyl, aminosulfonyl, sulfonylamino, sulfonyl, oxo and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

As used herein, the term “heteroatom” is meant to include oxygen (O), nitrogen (N), sulfur (S) and silicon (Si).

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

As used herein, a wavy line “ [symbol] ” that intersects a bond in a chemical structure indicates the point of attachment of the atom to which the wavy bond is connected in the chemical structure to the remainder of a molecule, or to the remainder of a fragment of a molecule.

As used herein, the representation of a group (e.g., X^{a}) in parenthesis followed by a subscript integer range (e.g., (X^{a})₀₋₁) means that the group can have the number of occurrences as designated by the integer range. For example, (X^{a})₀₋₁ means the group X^{a} can be absent or can occur one time.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the present disclosure. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water. Certain compounds of the present disclosure can exist in unsolvated forms as well as solvated forms, including hydrated forms. In general, the solvated forms are equivalent to unsolvated forms and are intended to be encompassed within the scope of the present disclosure.

The term “co-crystal” as used herein refers to a solid that is a crystalline single phase material composed of two or more different molecular or ionic compounds generally in a stoichiometric ratio which are neither solvates nor simple salts. A co-crystal consists of two or more components that form a unique crystalline structure having unique properties. Co-crystals are typically characterized by a crystalline structure, which is generally held together by freely reversible, non-covalent interactions. As used herein, a co-crystal refers to a compound of the present disclosure and at least one other component in a defined stoichiometric ratio that form a crystalline structure.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms are replaced by an atom having the atomic mass or mass number different from the predominant atomic mass or mass number usually found in nature for the atom. All isotopes of any particular atom or element as specified are contemplated within the scope of the compounds of the present disclosure and include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, 35S, ¹⁸F, ³⁶Cl, ¹²³I and ¹²⁵I. Certain isotopically labeled compounds of the present disclosure (e.g., those labeled with ³H or ¹⁴C) are useful in compound and/or substrate tissue distribution assays. Tritiated (³H) and carbon-14 (¹⁴C) isotopes are useful for their ease of preparation and detectability. Further substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present disclosure can generally be prepared by following procedures analogous to those disclosed in the Schemes and/or in the Examples herein below, by substituting an isotopically labeled reagent for a non-isotopically labeled reagent.

“Treating” or “treatment” of a disease in a patient refers to inhibiting the disease or arresting its development; or ameliorating or causing regression of the disease. As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the present disclosure contemplate any one or more of these aspects of treatment.

“Preventing”, “prevention”, or “prophylaxis” of a disease in a patient refers to preventing the disease from occurring in a patient that is predisposed or does not yet display symptoms of the disease.

The phrase “therapeutically effective amount” means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “cancer” and “cancerous” refer to or describe the physiological condition in mammals that is typically characterized by unregulated cell growth.

It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### II. Compounds

In one aspect, provided herein is a compound of formula (I),

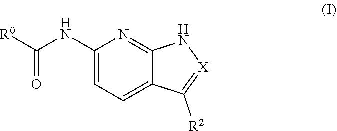

or a pharmaceutically acceptable salt, solvate, hydrate, or co-crystal thereof, or a mixture of any of the foregoing, wherein:
X is N or CR⁸;
R⁰ is a group

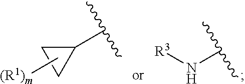

m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, —O—C₁-C₃ alkylene-NR⁴R⁵, —O—C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(4- to 8-membered heterocycloalkylene)-(C₀-C₂ alkylene)-NR⁴R⁵, C₁-C₂ alkylene-(4- to 8-membered heterocycloalkylene)-(C₀-C₂ alkylene)-NR^{4′}R⁵, C₁-C₂ alkylene-(C₃-C₇ heterocycloalkylene)-(C₀-C₂ alkylene)-NR⁴R⁵ or C₁-C₂ alkylene-(C₃-C₇ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl.
or
two R¹ are taken together with the carbon atom or carbon atoms to which they are attached to form a 3- to 7-membered heterocyclic ring, wherein the heterocyclic ring contains nitrogen atom and wherein the nitrogen atom is optionally substituted with C₁-C₃ alkyl;
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(4- to 8-membered heterocycloalkyl), C₁-C₃ alkylene-(C₃-C₇ heterocycloalkyl), C₁-C₃ alkylene-(4- to 8-membered heterocycloalkylene)-(C₀-C₂ alkylene)-NR⁴R⁵, C₁-C₃ alkylene-(4- to 8-membered heterocycloalkylene)-(C₀-C₂ alkylene)-NR^{4′}R^{5′}, (4- to 8-membered heterocycloalkylene)-(C₀-C₂ alkylene)-NR⁴R⁵, C₁-C₃ alkylene-(C₃-C₇ heterocycloalkylene)-(C₀-C₂ alkylene)-NR⁴R⁵, C₁-C₃ alkylene-(C₃-C₇ heterocycloalkylene)-(C₀-C₂ alkylene)-NR^{4′}R^{5′}, or (C₃-C₇ heterocycloalkylene)-(C₀-C₂ alkylene)-NR⁴R⁵, wherein the alkyl, alkylene, cycloalkyl, cycloalkylene, heterocycloalkyl, and heterocycloalkylene moieties in R³ are optionally substituted with 1-3 fluorine atoms, 1-3 CN groups and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, 4- to 8-membered heterocycloalkyl, or C₃-C₇ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each pair of R^{4′} and R^{5′} taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O)ₙC₁-C₃ alkyl, or S(O)ₙC₃-C₆ cycloalkyl, wherein n is an integer from 0 to 2;
each R⁷ is independently —H, C₁-C₃ alkyl, C₁-C₃ haloalkyl, C₃-C₆ cycloalkyl, or C₁-C₃-alkylene-C₃-C₆ cycloalkyl, wherein said C₁-C₃ alkyl is optionally substituted with 1-6 deuterium atoms; and
R⁸ is —H, —F, or C₁-C₃ alkyl.

In one aspect, provided herein is a compound of formula (I)

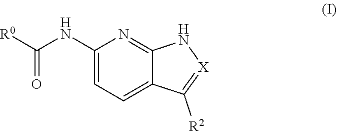

or a pharmaceutically acceptable salt, solvate, hydrate, or co-crystal thereof, or a mixture of any of the foregoing, wherein:
X is N or CR⁸;
R⁰ is a group

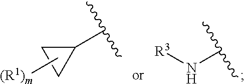

m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(C₄-C₆ heterocycloalkyl), C₁-C₃ alkylene-(C₄-C₆ heterocycloalkylene)-(C₀-C₂ alkylene)-NR⁴R⁵ or C₁-C₃ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O)ₙC₁-C₃ alkyl, or S(O)ₙC₃-C₆ cycloalkyl,
wherein n is an integer from 0 to 2;
each R⁷ is independently —H, C₁-C₃ alkyl, C₁-C₃ haloalkyl, or C₃-C₆ cycloalkyl, wherein said C₁-C₃ alkyl is optionally substituted with 1-6 deuterium atoms; and
R⁸ is —H, —F, or C₁-C₃ alkyl.

In some embodiments of the present aspect, R⁰ is

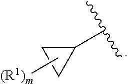

In some embodiments wherein R⁰ is

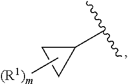

m is an integer 0, 1, 2 or 3. In some embodiments, m is 0. In other embodiments, m is 1. In yet other embodiments, m is 2. In still yet other embodiments, m is 3. In other embodiments of the present aspect, R⁰ is

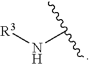

In some embodiments, the compound of formula (I) is a compound of formula (I-A) or formula (I-B)

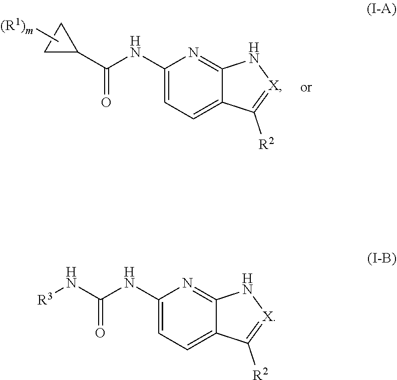

In some embodiments of the present aspect, X is N or CR⁸. In some embodiments, X is N. In some embodiments, X is CR⁸.

In some embodiments, each R¹ is independently -D, —F, C₁-C₃ alkyl, C₁-C₃ alkylene-NR⁴R⁵, C₁-C₃ alkylene-NR^{4′}R^{5′}, —O—C₁-C₃ alkylene-NR⁴R⁵, —O—C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(4- to 8-membered heterocycloalkylene)-(C₀-C₂ alkylene)-NR⁴R⁵, C₁-C₂ alkylene-(4- to 8-membered heterocycloalkylene)-(C₀-C₂ alkylene)-NR^{4′}R⁵, C₁-C₂ alkylene-(C₃-C₇ heterocycloalkylene)-(C₀-C₂ alkylene)-NR⁴R⁵ or C₁-C₂ alkylene-(C₃-C₇ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl.

In some embodiments, each R¹ is independently -D, —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, each R¹ is independently —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, each R¹ is independently -D, —F, or C₁-C₃ alkyl. In some embodiments, each R¹ is independently C₁-C₃ alkyl, C₁-C₃ alkylene-NR⁴R⁵, C₁-C₃ alkylene-NR^{4′}R^{5′}, —O—C₁-C₃ alkylene-NR⁴R⁵, —O—C₁-C₃ alkylene-NR^{4′}R^{5′}, C₁-C₃ alkylene-OH, or C₀-C₃ alkylene-CN. In some embodiments, each R¹ is independently C₁-C₃ alkyl, C₁-C₃ alkylene-NR⁴R⁵, C₁-C₃ alkylene-NR^{4′}R^{5′}, C₁-C₃ alkylene-OH, or C₀-C₃ alkylene-CN. In some embodiments, each R¹ is independently C₁-C₃ alkyl, C₁-C₃ alkylene-NR⁴R⁵, C₁-C₃ alkylene-NR^{4′}R^{5′}, —O—C₁-C₃ alkylene-NR⁴R⁵, or —O—C₁-C₃ alkylene-NR^{4′}R^{5′}. In some embodiments, each R¹ is independently C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(4- to 8-membered heterocycloalkylene)-(C₀-C₂ alkylene)-NR⁴R⁵, C₁-C₂ alkylene-(4- to 8-membered heterocycloalkylene)-(C₀-C₂ alkylene)-NR^{4′}R⁵, C₁-C₂ alkylene-(C₃-C₇ heterocycloalkylene)-(C₀-C₂ alkylene)-NR⁴R⁵ or C₁-C₂ alkylene-(C₃-C₇ heterocycloalkylene)-(C₀-C₂ alkylene)-NR^{4′}R^{5′}. In some embodiments, each R¹ is independently C₁-C₃ alkylene-NR⁴R⁵, C₁-C₃ alkylene-NR^{4′}R^{5′}, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}. In some embodiments, each R¹ is independently C₁-C₃ alkylene-OH, C₀-C₃ alkylene-CN, or C₁-C₂ alkylene-(C₃-C₆ cycloalkylene)-(C₀-C₂ alkylene)-OH. In some embodiments, each R¹ is independently —F, C₁-C₃ alkylene-NR^{4′}R^{5′}, C₁-C₃ alkylene-OH, or C₀-C₃ alkylene-CN, wherein each pair of R^{4′} and R^{5′} of R¹ taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, each R¹ is independently —F, C₁-C₃ alkylene-NR^{4′}R^{5′}, C₁-C₃ alkylene-OH, or C₀-C₃ alkylene-CN, wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl. In some embodiments, each R¹ is independently C₁-C₃ alkylene-NR^{4′}R^{5′}. In certain embodiments, each R¹ is independently optionally substituted —C₁-C₂ alkylene-N-morpholinyl or optionally substituted —C₁-C₂ alkylene-N-piperazinyl. In some embodiments, each R¹ is independently optionally substituted

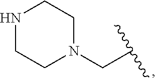 [SMILES: *CN1CCNCC1]

optionally substituted

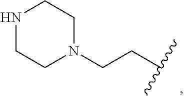 [SMILES: *CCN1CCNCC1]

optionally substituted

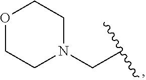 [SMILES: *CN1CCOCC1]

optionally substituted

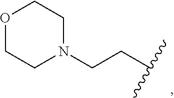 [SMILES: *CCN1CCOCC1]

optionally substituted

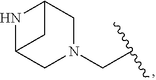 [SMILES: *CN1CC2CC(C1)N2]

or optionally substituted

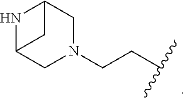 [SMILES: *CCN1CC2CC(C1)N2]

In certain embodiments, each R¹ is independently optionally substituted

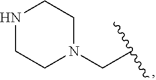 [SMILES: *CN1CCNCC1]

optionally substituted

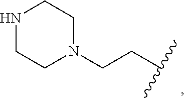 [SMILES: *CCN1CCNCC1]

optionally substituted

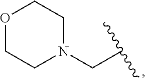 [SMILES: *CN1CCOCC1]

or optionally substituted

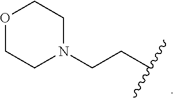 [SMILES: *CCN1CCOCC1]

In some embodiments, each R¹ is independently

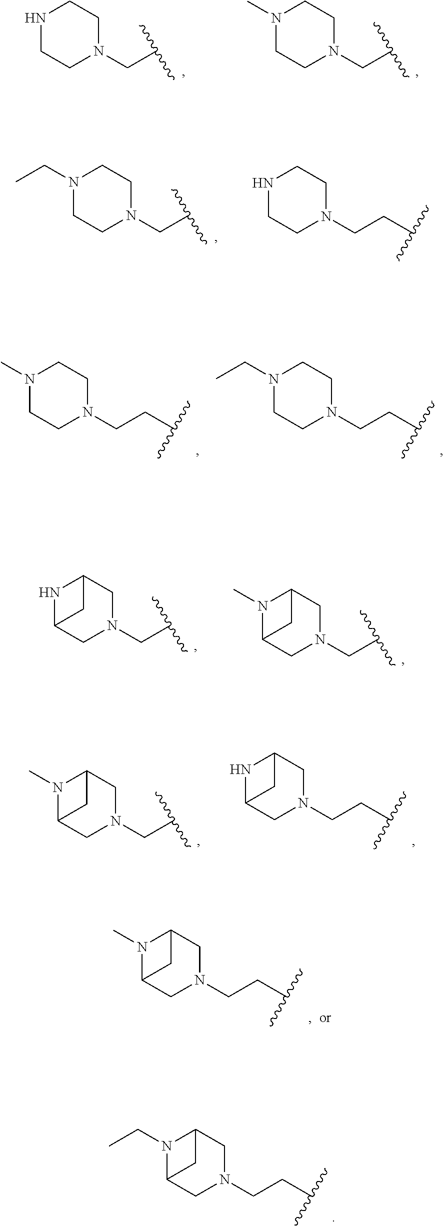

In certain embodiments, each R¹ is independently

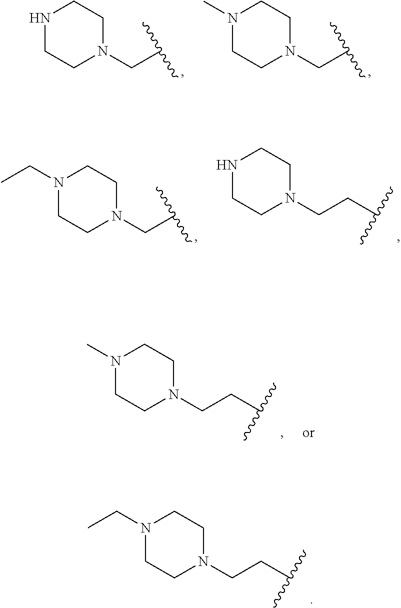

In certain other embodiments, each R¹ is independently,

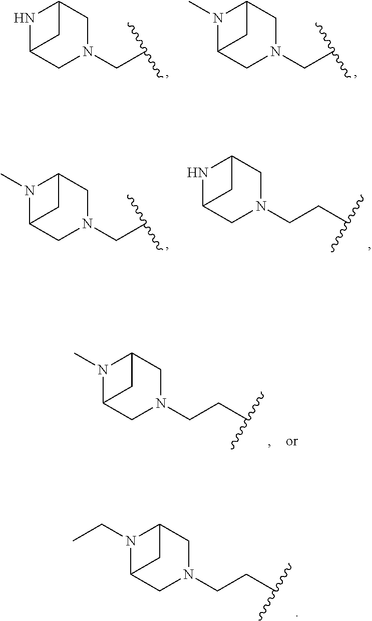

In still other embodiments, each R¹ is independently

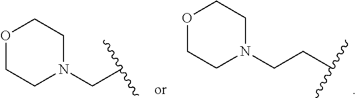 [SMILES: *CN1CCOCC1 ; *CCN1CCOCC1]

In some embodiments, each R¹ is independently C₁-C₃ alkylene-OH. In certain other embodiments, each R¹ is independently —C₁-C₂ alkylene-OH. In certain embodiments, each R¹ is independently —CH₂OH, —CH₂CH₂OH, —CH(OH)CH₃, —CH₂CH₂CH₂OH, —CH₂CH(OH)CH₃, or —CH(CN)CH₂CH₃. In certain other embodiments, each R¹ is independently —CH₂OH or —CH₂CH₂OH. In some embodiments, each R¹ is independently C₀-C₃ alkylene-CN. In certain embodiments, each R¹ is independently —CN, —CH₂CN, —CH₂CH₂CN, —CH(CN)CH₃, —CH₂CH₂CH₂CN, —CH₂CH(CN)CH₃, —CH(CN)CH₂CH₃, or —CH(CH₂CN)CH₃. In some embodiments, each R¹ is independently —F,

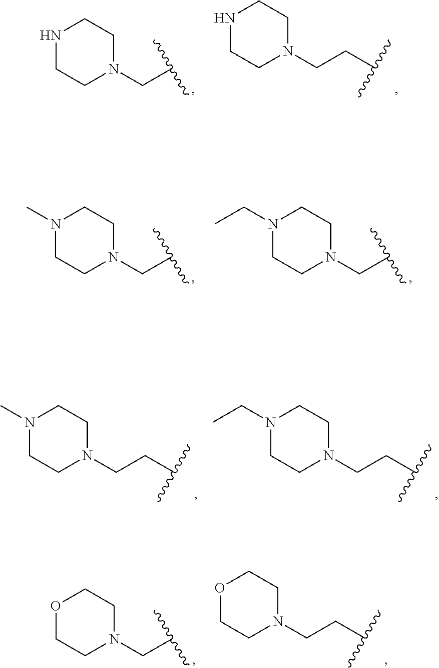

—CH₂OH, —CH₂CH₂OH, —CH₂—NHCH₃, —CH₂NHCD₃, —CH₂—N(CH₃)₂, —CH₂N(CD₃)₂, —CH₂CH₂—N(CH₃)₂, —CH₂CH₂N(CD₃)₂, —OCH₂CH₂N(CH₃)₂, —CN, or —CH₂CN. In some embodiments, each R¹ is independently —F,

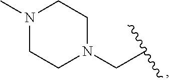 [SMILES: *CN1CCN(C)CC1]

—CH₂OH, —CH₂CH₂OH, —CN, or —CH₂CN.

In other embodiments, two R¹ are taken together with the carbon atom or carbon atoms to which they are attached to form a 3- to 7-membered heterocyclic ring, wherein the heterocyclic ring contains nitrogen atom and wherein the nitrogen atom is optionally substituted with C₁-C₃ alkyl.

For example, in some embodiments, two R¹ are present on adjacent carbon atoms and together with the carbon atoms to which they are attached form a 3- to 7-membered heterocyclic ring, such as a pyrrolidinyl ring, wherein the heterocyclic ring contains nitrogen atom and wherein the nitrogen atom is optionally substituted with C₁-C₃ alkyl. In other embodiments, two R¹ are present on the same carbon atom and together with the carbon atoms to which they are attached form a 3- to 7-membered heterocyclic ring, such as a azetidinyl ring, wherein the heterocyclic ring contains nitrogen atom and wherein the nitrogen atom is optionally substituted with C₁-C₃ alkyl.

In some embodiments, R² is C₆-C₁₄ aryl or 5-to-10-membered heteroaryl, wherein the C₆-C₁₄ aryl and 5-to-10-membered heteroaryl are optionally substituted with 1-5 R⁶ groups. In some embodiments, R² is a C₆-C₁₄ aryl, wherein said C₆-C₁₄ aryl is optionally substituted with 1-5 R⁶ groups. In some embodiments, R² is phenyl, wherein said phenyl is optionally substituted with 1-5 R⁶ groups. In some embodiments, R² is 5- to 10-membered heteroaryl, wherein said 5-to-10-membered heteroaryl is optionally substituted with 1-5 R⁶ groups. In some embodiments, R² is a 5-to-10-membered heteroaryl selected from the group consisting of

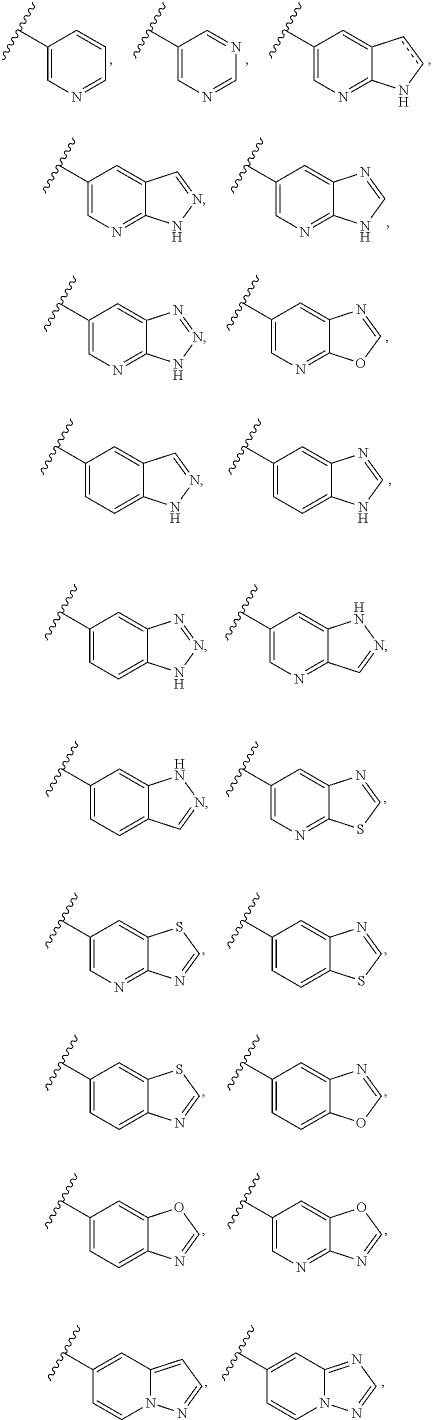

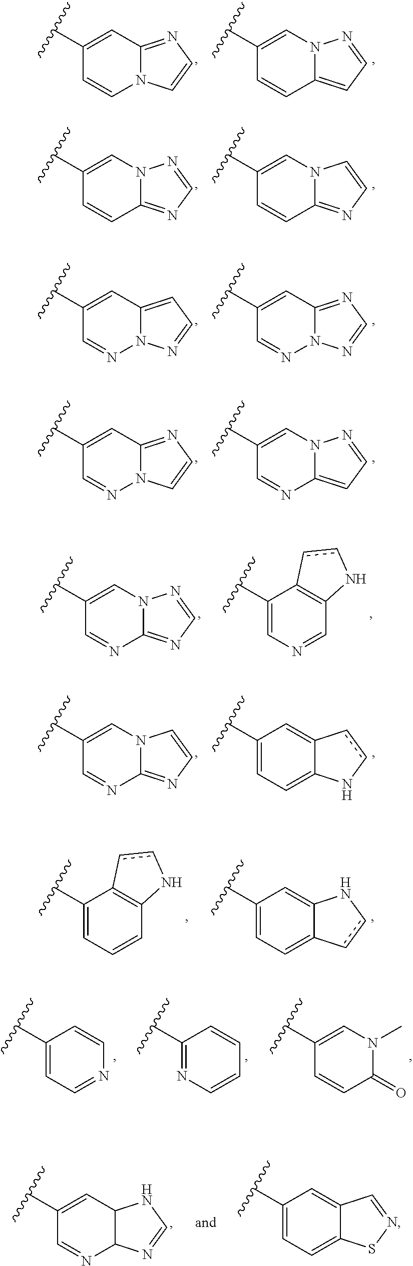

wherein [symbol] indicates a single or double bond, and wherein the 5-to-10-membered heteroaryl is optionally substituted with 1-5 R⁶ groups. In some embodiments, R² is

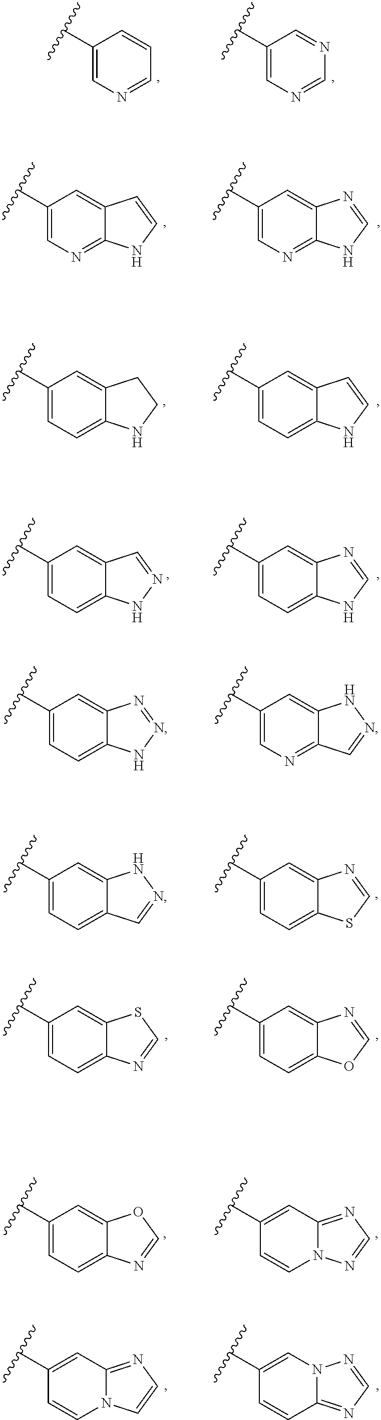

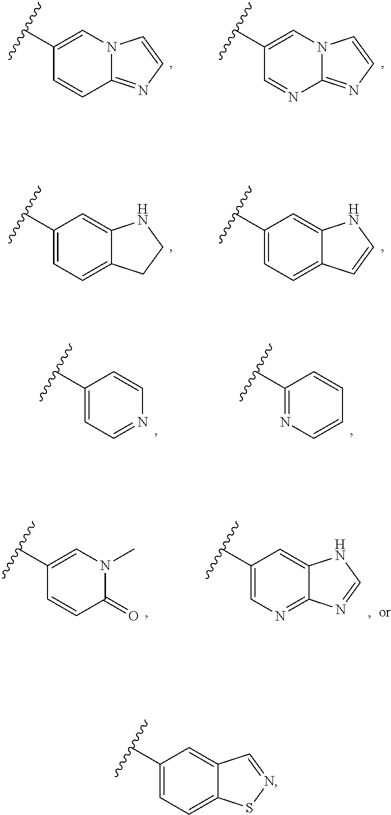

each of which is optionally substituted with 1-5 R⁶ groups. In some embodiments, R² is a 5-to-10-membered heteroaryl selected from the group consisting of

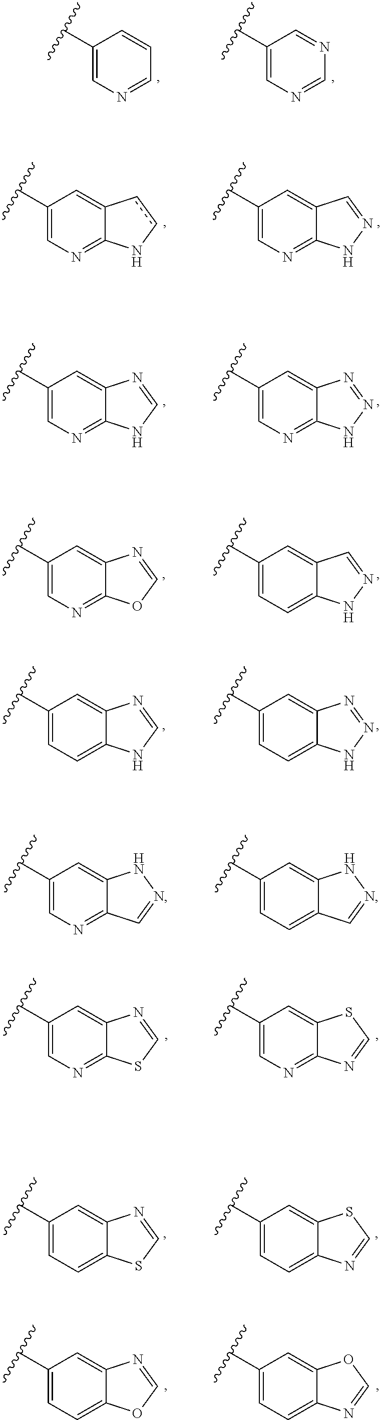

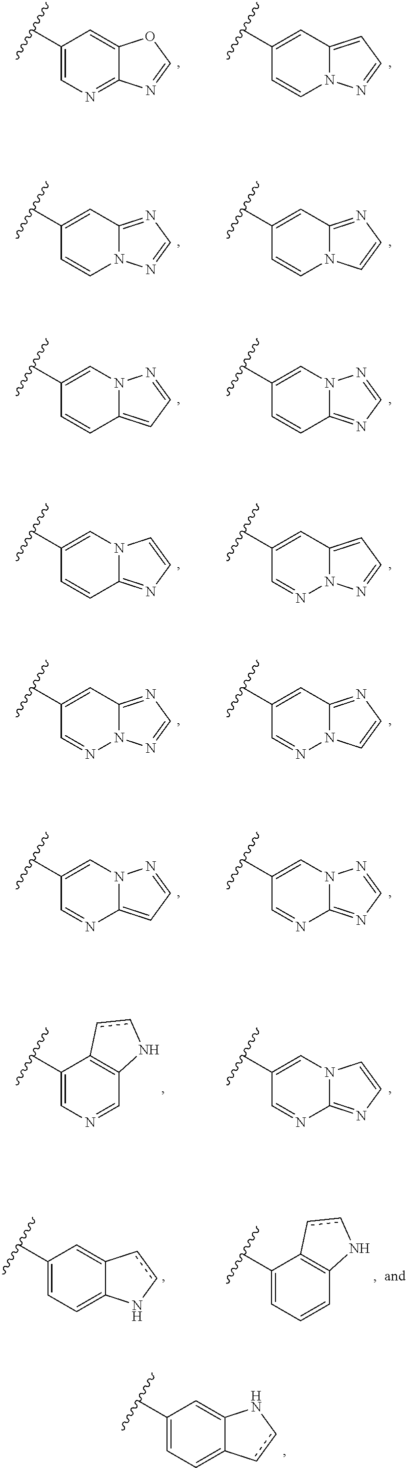

wherein [symbol] indicates a single or double bond, and wherein the 5-to-10-membered heteroaryl is optionally substituted with 1-5 R⁶ groups. In some embodiments, R² is

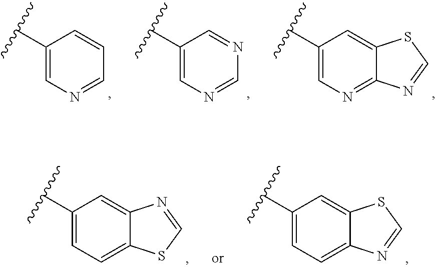 [SMILES: *c1cccnc1 ; *c1cncnc1 ; *c1cnc2ncsc2c1 ; *c1ccc2scnc2c1 ; *c1ccc2ncsc2c1]

each of which is optionally substituted with 1-5 R⁶ groups. In some embodiments, R² is phenyl,

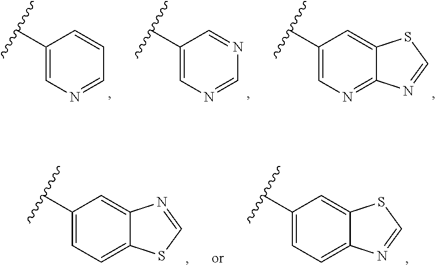 [SMILES: *c1cccnc1 ; *c1cncnc1 ; *c1cnc2ncsc2c1 ; *c1ccc2scnc2c1 ; *c1ccc2ncsc2c1]

each of which is optionally substituted with 1-5 R⁶ groups.

In some embodiments, R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(4- to 8-membered heterocycloalkyl), C₁-C₃ alkylene-(C₃-C₇ heterocycloalkyl), C₁-C₃ alkylene-(4- to 8-membered heterocycloalkylene)-(C₀-C₂ alkylene)-NR⁴R⁵, C₁-C₃ alkylene-(4- to 8-membered heterocycloalkylene)-(C₀-C₂ alkylene)-NR^{4′}R^{5′}, (4- to 8-membered heterocycloalkylene)-(C₀-C₂ alkylene)-NR⁴R⁵, C₁-C₃ alkylene-(C₃-C₇ heterocycloalkylene)-(C₀-C₂ alkylene)-NR⁴R⁵, C₁-C₃ alkylene-(C₃-C₇ heterocycloalkylene)-(C₀-C₂ alkylene)-NR^{4′}R^{5′}, or (C₃-C₇ heterocycloalkylene)-(C₀-C₂ alkylene)-NR⁴R⁵, wherein the alkyl, alkylene, cycloalkyl, cycloalkylene, heterocycloalkyl, and heterocycloalkylene moieties in R³ are optionally substituted with 1-3 fluorine atoms, 1-3 CN groups and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, 4- to 8-membered heterocycloalkyl, or C₃-C₇ heterocycloalkyl.

In some embodiments, R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆ alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′}R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₂ alkylene-(C₃-C₆ cycloalkylene)-(C₀-C₂ alkylene)-OH, C₁-C₃ alkylene-(C₄-C₆ heterocycloalkyl), C₁-C₃ alkylene-(C₄-C₆ heterocycloalkylene)-(C₀-C₂ alkylene)-NR⁴R⁵ or C₁-C₃ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl.

In some embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-OH, C₁-C₃ alkylene-CN, C₁-C₂ alkylene-(C₃-C₆ cycloalkyl), C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-O—(C₁-C₂ alkylene)-NR⁴R⁵, C₁-C₂ alkylene-(C₃-C₆ cycloalkylene)-(C₀-C₂ alkylene)-OH, C₁-C₂ alkylene-(C₄-C₆ heterocycloalkyl), C₁-C₂ alkylene-(C₄-C₆ heterocycloalkylene)-(C₀-C₂ alkylene)-NR⁴R⁵, or C₁-C₂ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl. In some embodiments, R³ is C₁-C₃ alkylene-(4- to 8-membered heterocycloalkyl), C₁-C₃ alkylene-(C₃-C₇ heterocycloalkyl), C₁-C₃ alkylene-(4- to 8-membered heterocycloalkylene)-(C₀-C₂ alkylene)-NR⁴R⁵, C₁-C₃ alkylene-(4- to 8-membered heterocycloalkylene)-(C₀-C₂ alkylene)-NR^{4′}R^{5′}, (4- to 8-membered heterocycloalkylene)-(C₀-C₂ alkylene)-NR⁴R⁵, C₁-C₃ alkylene-(C₃-C₇ heterocycloalkylene)-(C₀-C₂ alkylene)-NR⁴R⁵, C₁-C₃ alkylene-(C₃-C₇ heterocycloalkylene)-(C₀-C₂ alkylene)-NR^{4′}R^{5′}, or (C₃-C₇ heterocycloalkylene)-(C₀-C₂ alkylene)-NR⁴R⁵ In some embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, C₁-C₃ alkylene-(4- to 8-membered heterocycloalkyl), or C₁-C₃ alkylene-(C₃-C₇ heterocycloalkyl), wherein each heterocyclic nitrogen atom, if present, is independently optionally substituted with C₁-C₃ alkyl or C₄-C₆ heterocycloalkyl, wherein the R⁴ and R⁵ of R³ taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, or C₁-C₂ alkylene-(C₄-C₆ heterocycloalkyl), wherein each heterocyclic nitrogen atom, if present, is independently optionally substituted with C₁-C₃ alkyl or C₄-C₆ heterocycloalkyl, wherein the R⁴ and R⁵ of R³ are independently —H or C₁-C₃ alkyl, and the R^{4′} and R^{5′} of R³ taken together with the nitrogen atom to which they are attached form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl. In some embodiments, each R¹ is independently optionally substituted

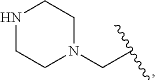 [SMILES: *CN1CCNCC1]

optionally substituted

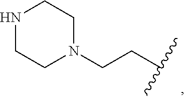 [SMILES: *CCN1CCNCC1]

optionally substituted

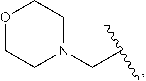 [SMILES: *CN1CCOCC1]

optionally substituted

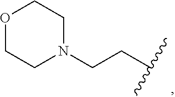 [SMILES: *CCN1CCOCC1]

optionally substituted

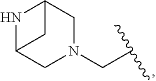 [SMILES: *CN1CC2CC(C1)N2]

or optionally substituted

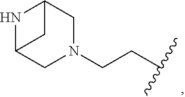 [SMILES: *CCN1CC2CC(C1)N2]

In certain embodiments, each R¹ is independently optionally substituted

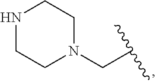 [SMILES: *CN1CCNCC1]

optionally substituted

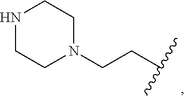 [SMILES: *CCN1CCNCC1]

optionally substituted

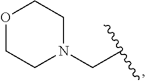 [SMILES: *CN1CCOCC1]

or optionally substituted

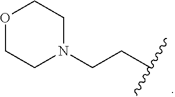 [SMILES: *CCN1CCOCC1]

In some embodiments, each R¹ is independently

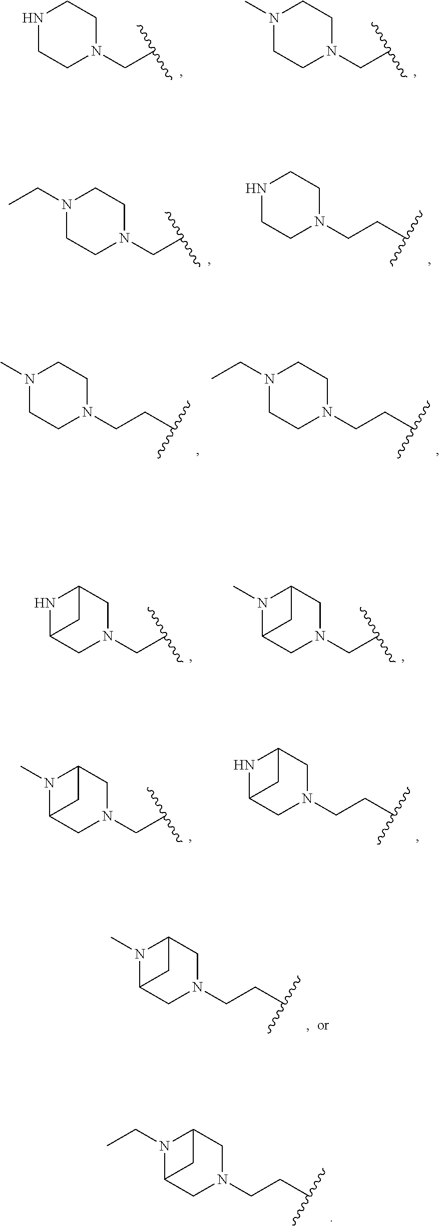

In certain embodiments, each R¹ is independently

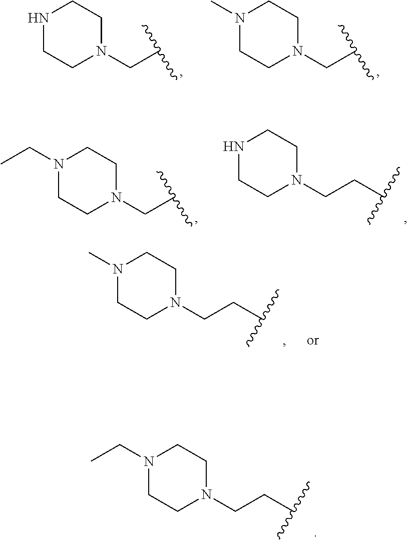

In certain embodiments, each R¹ is independently

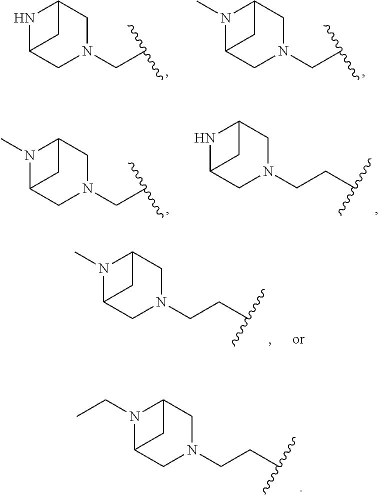

In some embodiments R³ is,

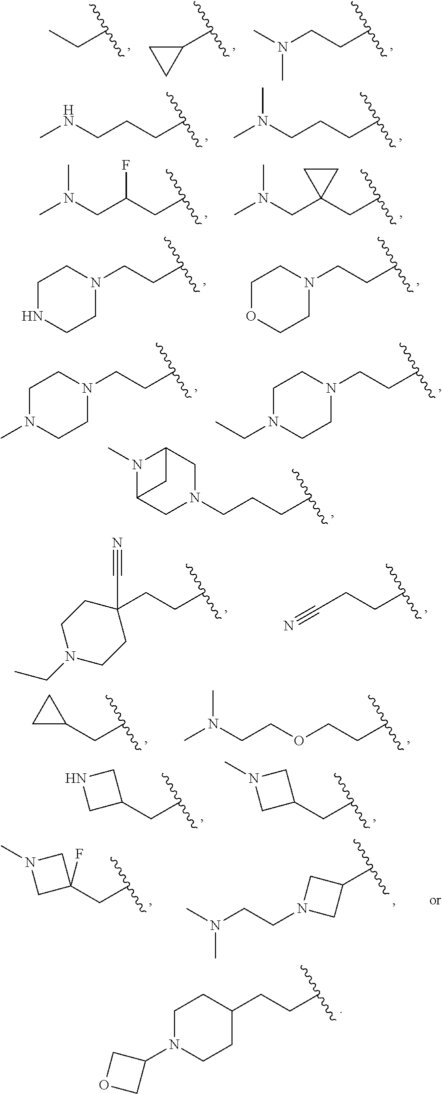

In some embodiments, R³ is

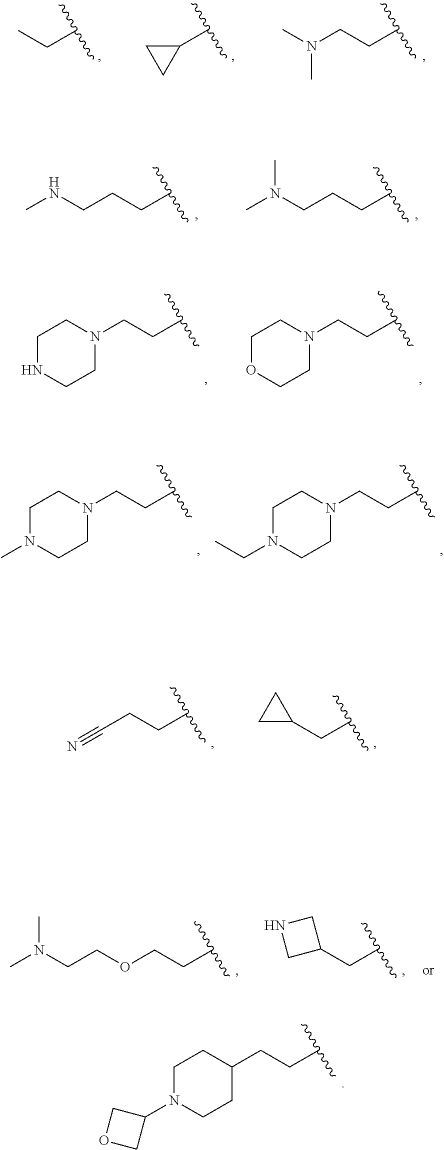

In some embodiments, each R⁴ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms. In some embodiments, each R⁴ is independently —H, C₁-C₆ alkyl, C₃-C₆ cycloalkyl, C₁-C₆ haloalkyl, C₁-C₆ alkylene-CN, or C₁-C₆ heteroalkyl. In some embodiments, each R⁴ is independently —H, C₁-C₃ alkyl, C₃-C₆ cycloalkyl, C₂-C₃ haloalkyl, C₂-C₃ alkylene-CN, or C₂-C₃ heteroalkyl. In some embodiments, each R⁴ is independently —H. In some embodiments, each R⁴ is independently C₁-C₃ alkyl. In certain embodiments, each R⁴ is independently —CH₃, —CH₂CH₃, —CH₂CH₂CH₃, or —CH(CH₃)₂. In some embodiments, each R⁴ is independently C₃-C₆ cycloalkyl. In certain embodiments, R⁴ is independently cyclopropyl, cyclobutyl, cyclopentyl or cyclohexyl. In some embodiments, each R⁴ is independently C₂-C₃ haloalkyl. In certain embodiment, each R⁴ is independently C₂-C₃ haloalkyl, wherein the each halogen atom of each C₂-C₃ haloalkyl is independently —F, —C₁, or —Br. In some embodiments, each R⁴ is independently C₂-C₃ alkylene-CN. In certain embodiments, each R⁴ is independently —CH₂CH₂CN, —CH(CN)CH₃, —CH₂CH₂CH₂CN, —CH₂CH(CN)CH₃, —CH(CN)CH₂CH₃, or —CH(CH₂CN)CH₃. In some embodiments, each R⁴ is independently C₂-C₃ heteroalkyl. In certain embodiments, each R⁴ is independently —CH₂CH₂OH, —CH(OH)CH₃, —CH₂CH₂CH₂OH, —CH₂CH(OH)CH₃, or —CH(OH)CH₂CH₃, —CH₂OCH₃, —CH₂OCH₂CH₃, —CH₂CH₂OCH₃, —CH(OCH₃)CH₃, —CH(CH₂OH)CH₃, —CH₂CH₂ NH₂, —CH(NH₂)CH₃, —CH₂CH₂CH₂ NH₂, —CH₂CH(NH₂)CH₃, or —CH(NH₂)CH₂CH₃, —CH₂NHCH₃, —CH₂NHCH₂CH₃, —CH₂CH₂NHCH₃, —CH(NHCH₃)CH₃, —CH(CH₂NH₂CH₃, —CH₂CH₂SH, —CH(SH)CH₃, —CH₂CH₂CH₂SH, —CH₂CH(SH)CH₃, or —CH(SH)CH₂CH₃, —CH₂SCH₃, —CH₂SCH₂CH₃, —CH₂CH₂SCH₃, —CH(SCH₃)CH₃, or —CH(CH₂SH)CH₃.

In some embodiments, each R⁵ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms. In some embodiments, each R⁵ is independently —H, C₁-C₆ alkyl, C₃-C₆ cycloalkyl, C₁-C₆ haloalkyl, C₁-C₆ alkylene-CN, or C₁-C₆ heteroalkyl. In some embodiments, each R⁵ is independently —H, C₁-C₃ alkyl, C₃-C₆ cycloalkyl, C₂-C₃ haloalkyl, C₂-C₃ alkylene-CN, or C₂-C₃ heteroalkyl. In some embodiments, each R⁵ is independently —H. In some embodiments, each R⁵ is independently C₁-C₃ alkyl. In certain embodiments, each R⁵ is independently —CH₃, —CH₂CH₃, —CH₂CH₂CH₃, or —CH(CH₃)₂. In some embodiments, each R⁵ is independently C₃-C₆ cycloalkyl. In certain embodiments, R⁵ is cyclopropyl, cyclobutyl, cyclopentyl or cyclohexyl. In some embodiments, each R⁵ is independently C₂-C₃ haloalkyl. In certain embodiment, each R⁵ is independently C₂-C₃ haloalkyl, wherein the each halogen atom of each C₂-C₃ haloalkyl is independently —F, —C₁, or —Br. In some embodiments, each R⁵ is independently C₂-C₃ alkylene-CN. In certain embodiments, each R⁵ is independently —CH₂CH₂CN, —CH(CN)CH₃, —CH₂CH₂CH₂CN, —CH₂CH(CN)CH₃, —CH(CN)CH₂CH₃, or —CH(CH₂CN)CH₃. In some embodiments, each R⁵ is independently C₂-C₃ heteroalkyl. In certain embodiments, each R⁵ is independently —CH₂CH₂OH, —CH(OH)CH₃, —CH₂CH₂CH₂OH, —CH₂CH(OH)CH₃, or —CH(OH)CH₂CH₃, —CH₂OCH₃, —CH₂OCH₂CH₃, —CH₂CH₂OCH₃, —CH(OCH₃)CH₃, —CH(CH₂OH)CH₃, —CH₂CH₂ NH₂, —CH(NH₂)CH₃, —CH₂CH₂CH₂ NH₂, —CH₂CH(NH₂)CH₃, or —CH(NH₂)CH₂CH₃, —CH₂NHCH₃, —CH₂NHCH₂CH₃, —CH₂CH₂NHCH₃, —CH(NHCH₃)CH₃, —CH(CH₂NH₂CH₃, —CH₂CH₂SH, —CH(SH)CH₃, —CH₂CH₂CH₂SH, —CH₂CH(SH)CH₃, or —CH(SH)CH₂CH₃, —CH₂SCH₃, —CH₂SCH₂CH₃, —CH₂CH₂SCH₃, —CH(SCH₃)CH₃, or —CH(CH₂SH)CH₃.

In some embodiments, each pair of R^{4′} and R^{5′} taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl. In some embodiments, each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl.

In some embodiments, each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O)ₙC₁-C₃ alkyl, or S(O)ₙC₃-C₆ cycloalkyl, wherein n is an integer from 0 to 2. In some embodiments, each R⁶ is independently halogen, —OR⁷, —NR⁴R⁵, C₁-C₃ alkyl, —CF₂H, —CF₃, C₃-C₆ cycloalkyl, or —CN. In some embodiments, each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl. In some embodiments, each R⁶ is independently —F, —Cl, —OH, —OCH₃, —OCH₂CH₃, —OCF₃, —OCF₂H, —OCH₂CF₃, —OCD₃, cyclopropyloxy, cyclobutoxy, —O—CH₂-cyclopropyl, —NH₂, —NHCH₃, —N(CH₃)₂, —CH₃, —CF₃, or —CN. In some embodiments, each R⁶ is independently —F, —Cl, —OH, —OCH₃, —OCH₂CH₃, —OCF₃, —OCF₂H, —OCH₂CF₃, —OCD₃, cyclopropyloxy, —NH₂, —NHCH₃, —N(CH₃)₂, —CH₃, —CF₃, or —CN. In some embodiments, each R⁶ is independently halogen or —OR⁷. In some embodiments, each R⁶ is independently —F, —OCH₃, or cyclopropyloxy.

In some embodiments, each R⁷ is independently —H, C₁-C₃ alkyl, C₁-C₃ haloalkyl, C₃-C₆ cycloalkyl, or C₁-C₃-alkylene-C₃-C₆ cycloalkyl, wherein said C₁-C₃ alkyl is optionally substituted with 1-6 deuterium atoms. In some embodiments, each R⁷ is independently —H, C₁-C₃ alkyl, C₁-C₃ haloalkyl, or C₃-C₆ cycloalkyl, wherein said C₁-C₃ alkyl is optionally substituted with 1-6 deuterium atoms. In some embodiments, each R⁷ is independently —H, C₁-C₃ alkyl, —CD₃, C₁-C₂ haloalkyl, or C₃-C₆ cycloalkyl. In some embodiments, each R⁷ is independently —H, C₁-C₃ alkyl, —CD₃, —CF₂H, —CF₃, or C₃-C₆ cycloalkyl. In some embodiments, each R⁷ is independently —H, C₁-C₂ alkyl, —CD₃, C₁-C₂ haloalkyl, or C₃ cycloalkyl. In some embodiments, each R⁷ is independently C₁-C₂ alkyl or C₃ cycloalkyl.

In some embodiments, R⁸ is —H, —F, or C₁-C₃ alkyl. In some embodiments, R⁸ is —H, —F, or —CH₃. In some embodiments, R⁸ is —H or —F. In some embodiments, R⁸ is —H or C₁-C₃ alkyl. In some embodiments, R⁸ is —F or C₁-C₃ alkyl. In some embodiments, R⁸ is —H, —F, —CH₃, —CH₂CH₃, —CH₂CH₂CH₃, or —CH(CH₃)₂. In some embodiments, R⁸ is —H. In some embodiments, R⁸ is —F. In some embodiments, R⁸ is C₁-C₃ alkyl. In some embodiments, —CH₃, —CH₂CH₃, —CH₂CH₂CH₃, or —CH(CH₃)₂. In some embodiments, R¹ is —CH₃.

In some embodiments, the compound of formula (I) is a compound of formula (I-A), or a pharmaceutically acceptable salt, solvate, hydrate, or co-crystal thereof, or a mixture of any of the foregoing

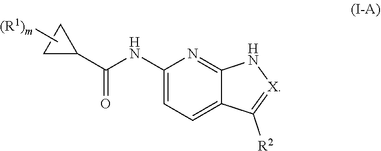

In some embodiments, the compound of formula (I) or formula (I-A) is a compound of formula (I-A-i) or formula (I-A-ii)

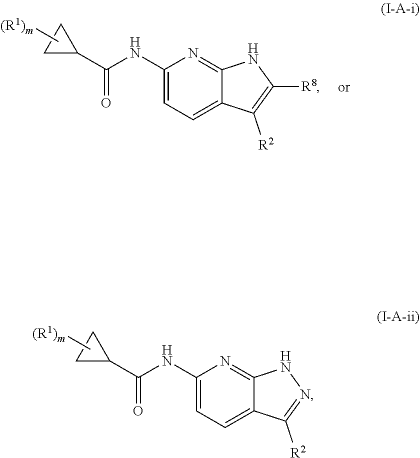

wherein
m is an integer from 0 to 2;
each R¹ is independently —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
R² is C₆-C₁₄ aryl or 5-to-10-membered heteroaryl, wherein said 5-to-10-membered heteroaryl is selected from the group consisting of

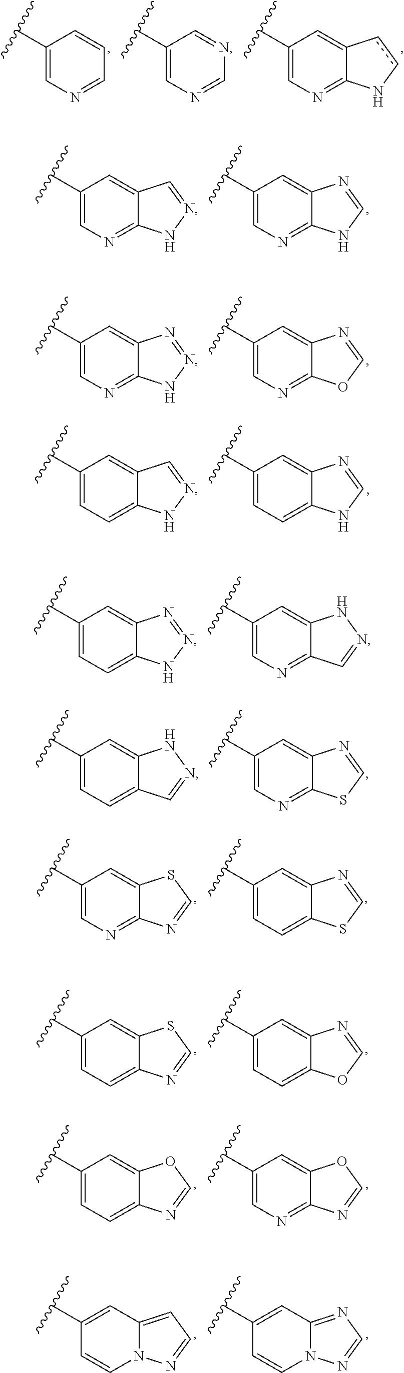

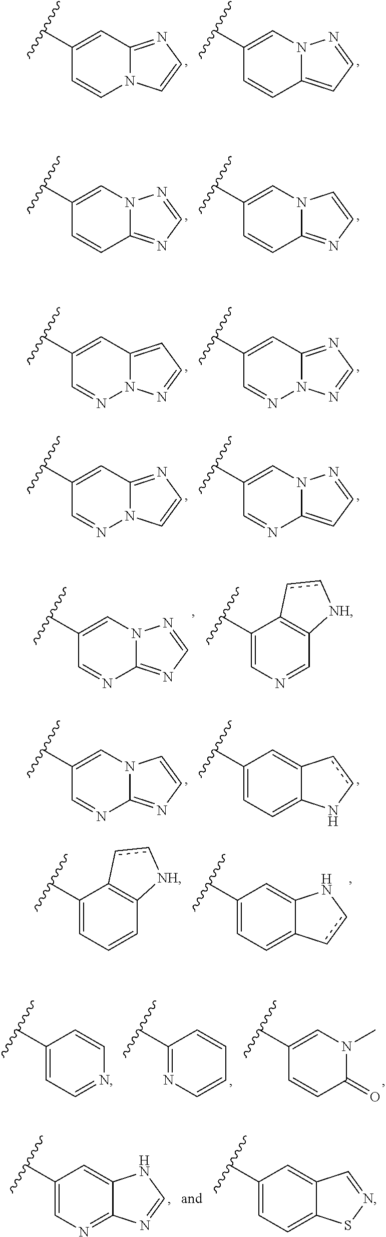

wherein [symbol] indicates a single or double bond, and wherein the C₆-C₁₄ aryl and 5-to-10-membered heteroaryl are optionally substituted with 1-5 R⁶ groups;
each R⁴ is independently —H, C₁-C₃ alkyl, C₃-C₆ cycloalkyl, C₂-C₃ haloalkyl, C₂-C₃ alkylene-CN, or C₂-C₃ heteroalkyl;
each R⁵ is independently —H, C₁-C₃ alkyl, C₃-C₆ cycloalkyl, C₂-C₃ haloalkyl, C₂-C₃ alkylene-CN, or C₂-C₃ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, C₁-C₃ alkyl, —CF₂H, —CF₃, C₃-C₆ cycloalkyl, or —CN;
each R⁷ is independently —H, C₁-C₃ alkyl, —CD₃, —CF₂H, —CF₃, or C₃-C₆ cycloalkyl; and
R⁸ is —H, —F, or —CH₃.

In some embodiments of the compound of formula (I), formula (I-A), formula (I-A-i),
each R¹ is independently —F, C₁-C₃ alkylene-NR^{4′}R^{5′}, C₁-C₃ alkylene-OH, or C₀-C₃ alkylene-CN,
wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl.

In some embodiments, R² is phenyl,

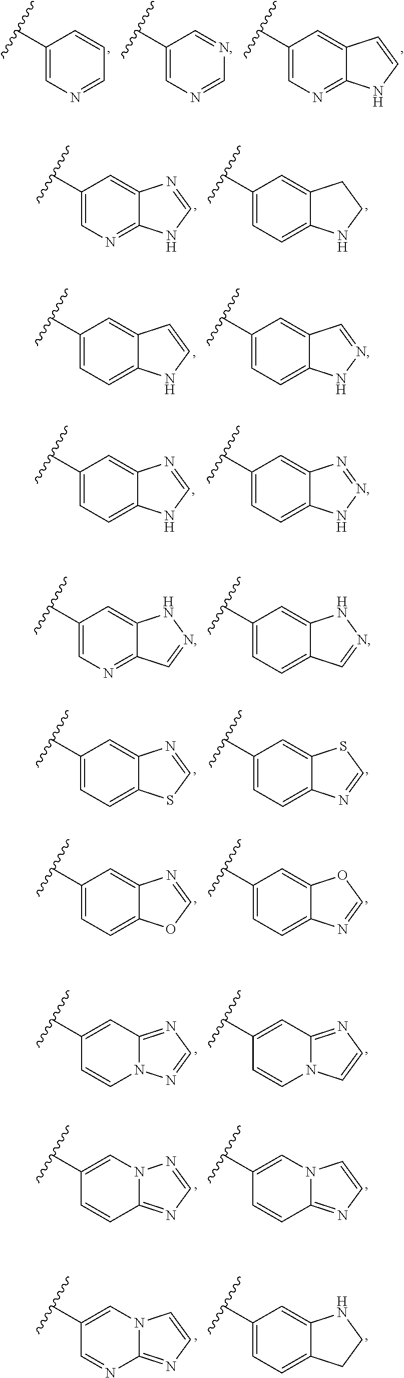

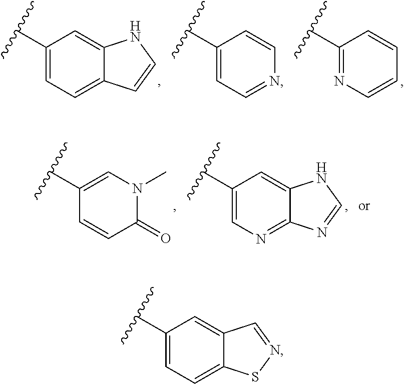

each of which is optionally substituted with 1-5 R⁶ groups.

In some embodiments, each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl; and each R⁷ is independently —H, C₁-C₂ alkyl, —CD₃, C₁-C₂ haloalkyl, or C₃ cycloalkyl.

In some embodiments, each R¹ is independently —F, C₁-C₃ alkylene-NR^{4′}R^{5′}, C₁-C₃ alkylene-OH, or C₀-C₃ alkylene-CN, wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl;
R² is phenyl,

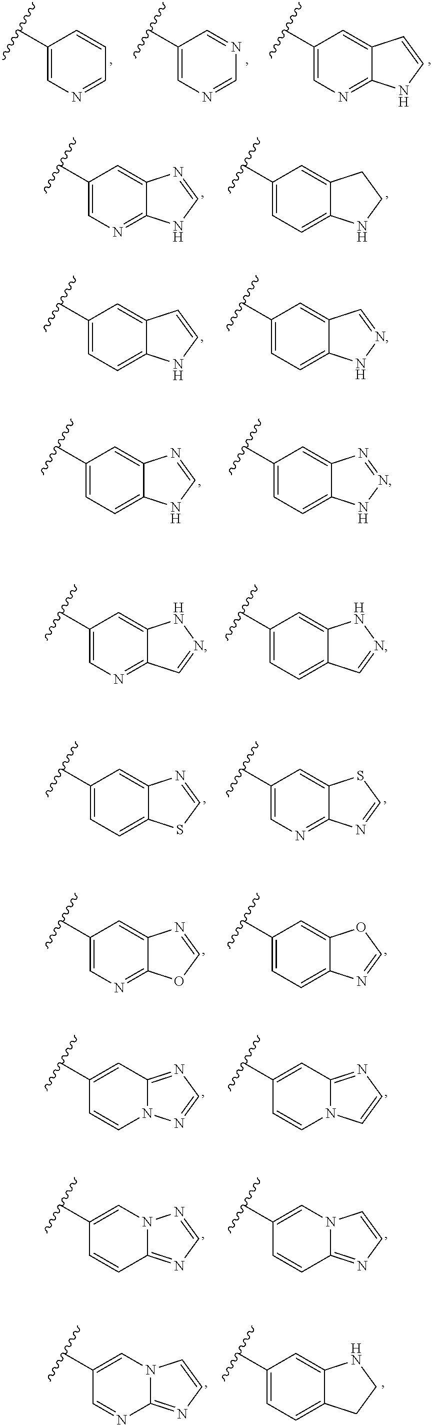

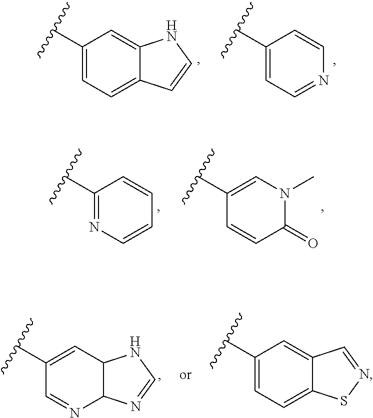

each of which is optionally substituted with 1-5 R⁶ groups;
each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl; and
each R⁷ is independently —H, C₁-C₂ alkyl, —CD₃, C₁-C₂ haloalkyl, or C₃ cycloalkyl.

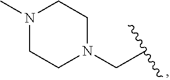 [SMILES: *CN1CCN(C)CC1]

In some embodiments, each R¹ is independently F, N—CH₂OH, —CH₂CH₂OH, —CN, or —CH₂CN.

In some embodiments of the compound of formula (I), formula (I-A), formula (I-A-i), each R⁶ is independently —F, —Cl, —OH, —OCH₃, —OCH₂CH₃, —OCF₃, —OCF₂H, —OCH₂CF₃, OCD₃, cyclopropyloxy, —NH₂, —NHCH₃, —N(CH₃)₂, —CH₃, —CF₃, or —CN.

In some embodiments, the compound of formula (I) is a compound of formula (I-B), or a pharmaceutically acceptable salt, solvate, hydrate, or co-crystal thereof, or a mixture of any of the foregoing

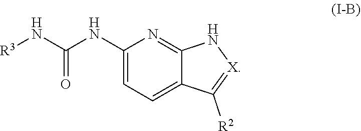

In some embodiments, wherein the compound of formula (I) or formula (I-B) is a compound of formula (I-B-i) or formula (I-B-ii)

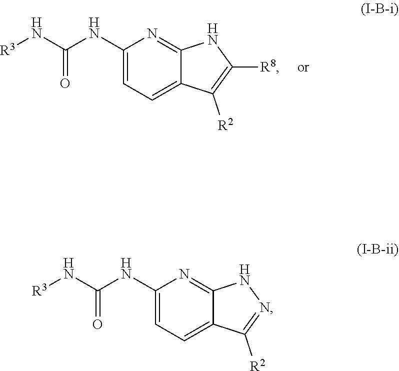

wherein:
R² is C₆-C₁₄ aryl or 5-to-10-membered heteroaryl, wherein said 5-to-10-membered heteroaryl is selected from the group consisting of

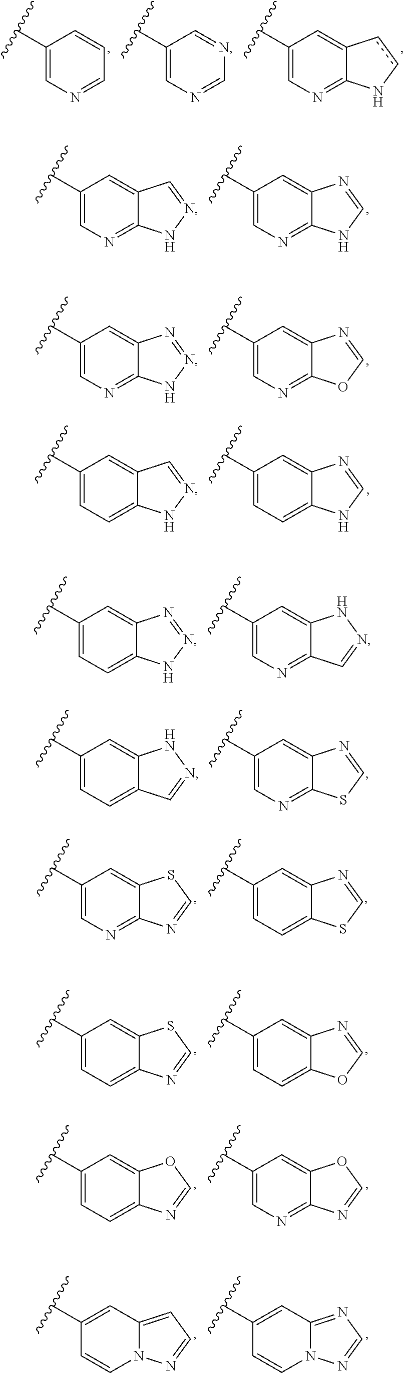

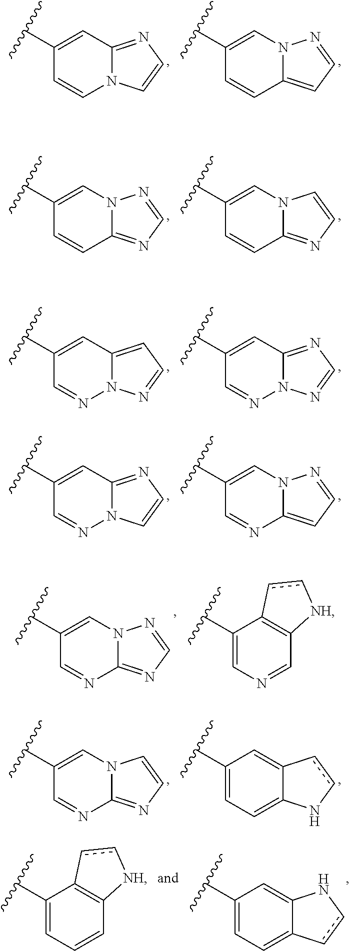

wherein [symbol] indicates a single or double bond, and wherein the C₆-C₁₄ aryl and 5-to-10-membered heteroaryl are optionally substituted with 1-5 R⁶ groups;
R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-OH, C₁-C₃ alkylene-CN, C₁-C₂ alkylene-(C₃-C₆ cycloalkyl), C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-O—(C₁-C₂ alkylene)-NR⁴R⁵, C₁-C₂ alkylene-(C₃-C₆ cycloalkylene)-(C₀-C₂ alkylene)-OH, C₁-C₂ alkylene-(C₄-C₆ heterocycloalkyl), C₁-C₂ alkylene-(C₄-C₆ heterocycloalkylene)-(C₀-C₂ alkylene)-NR⁴R⁵, or C₁-C₂ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl;
each R⁴ is independently —H, C₁-C₃ alkyl, C₃-C₆ cycloalkyl, C₂-C₃ haloalkyl, C₂-C₃ alkylene-CN, or C₂-C₃ heteroalkyl;
each R⁵ is independently —H, C₁-C₃ alkyl, C₃-C₆ cycloalkyl, C₂-C₃ haloalkyl, C₂-C₃ alkylene-CN, or C₂-C₃ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, C₁-C₃ alkyl, —CF₂H, —CF₃, C₃-C₆ cycloalkyl, or —CN;
each R⁷ is independently —H, C₁-C₃ alkyl, —CD₃, —CF₂H, —CF₃, or C₃-C₆ cycloalkyl; and
R⁸ is —H, —F, or —CH₃.

In some embodiments, R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, or C₁-C₂ alkylene-(C₄-C₆ heterocycloalkyl), wherein each heterocyclic nitrogen atom, if present, is independently optionally substituted with C₁-C₃ alkyl or C₄-C₆ heterocycloalkyl, wherein the R⁴ and R⁵ of R³ are independently —H or C₁-C₃ alkyl, and the R^{4′} and R^{5′} of R³ taken together with the nitrogen atom to which they are attached form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl.

In some embodiments,
R² is phenyl,

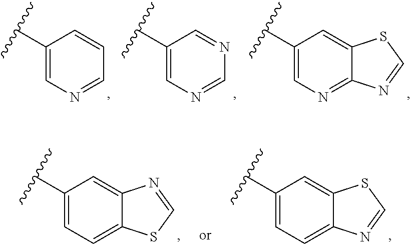

each of which is optionally substituted with 1-5 R⁶ groups.

In some embodiments, each R⁶ is independently halogen or —OR⁷; and
each R⁷ is independently C₁-C₂ alkyl or C₃ cycloalkyl.

In some embodiments of the compound of formula (I-B), formula (I-B-i) or formula (I-B-ii), R³ is C₁-C₃ alkyl, C₃-C₆ cycloalkyl, C₁-C₃ alkylene-NR⁴R⁵, C₁-C₃ alkylene-NR^{4′}R^{5′}, C₁-C₃ alkylene-CN, C₁-C₂ alkylene-(C₃-C₆ cycloalkyl), C₁-C₂ alkylene-O—(C₁-C₂ alkylene)-NR⁴R⁵, or C₁-C₂ alkylene-(C₄-C₆ heterocycloalkyl), wherein each heterocyclic nitrogen atom, if present, is independently optionally substituted with C₁-C₃ alkyl or C₄-C₆ heterocycloalkyl,
wherein the R⁴ and R⁵ of R³ are independently —H or C₁-C₃ alkyl, and
the R^{4′} and R^{5′} of R³ taken together with the nitrogen atom to which they are attached form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl;
R² is phenyl,

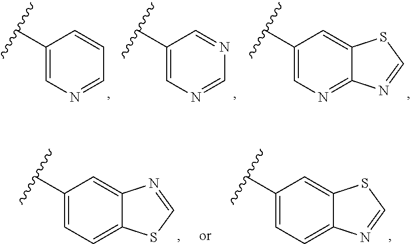

each of which is optionally substituted with 1-5 R⁶ groups;
each R⁶ is independently halogen or —OR⁷; and
each R⁷ is independently C₁-C₂ alkyl or C₃ cycloalkyl.

In some embodiments, R³ is

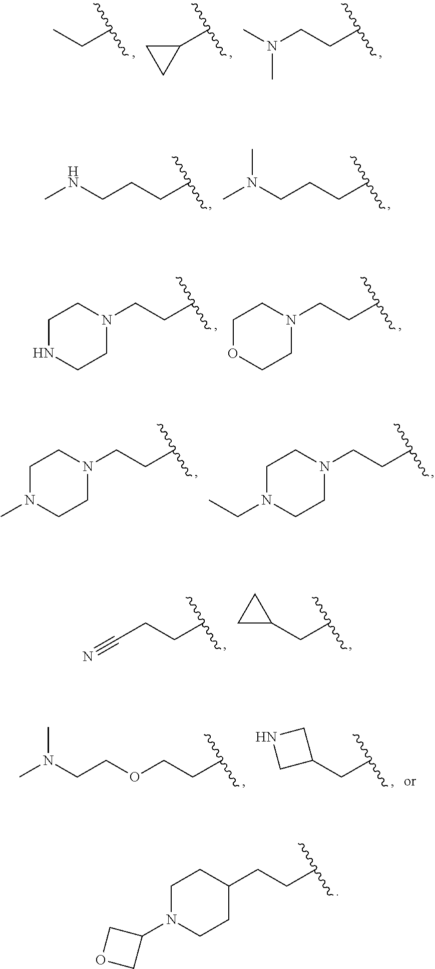

In some embodiments, each R⁶ is independently —F, —OCH₃, or cyclopropyloxy. In some embodiments, R⁸ is H.

In some embodiments, provided is a compound selected from the compounds in Table 1, or a pharmaceutically acceptable salt, solvate, hydrate, or co-crystal thereof, or a mixture of any of the foregoing.

TABLE 1

| Cmpd | |
|---|---|
| No. | Structure |
| 1 | 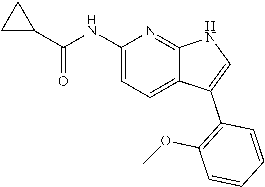 [SMILES: COc1ccccc1-c1c[nH]c2nc(NC(=O)C3CC3)ccc12] |
| 2 | 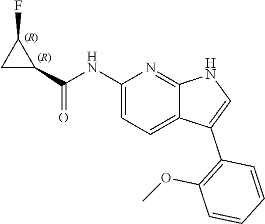 |
| 2-1 | 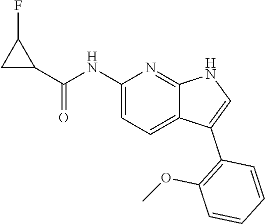 [SMILES: COc1ccccc1-c1c[nH]c2nc(NC(=O)C3CC3F)ccc12] |
| 3 | 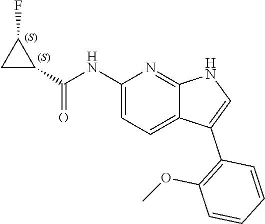 |
| 2-1 | 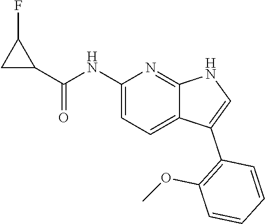 [SMILES: COc1ccccc1-c1c[nH]c2nc(NC(=O)C3CC3F)ccc12] |
| 4 | 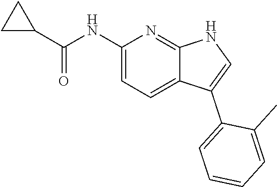 [SMILES: Cc1ccccc1-c1c[nH]c2nc(NC(=O)C3CC3)ccc12] |
| 5 | 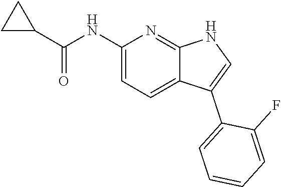 [SMILES: O=C(Nc1ccc2c(-c3ccccc3F)c[nH]c2n1)C1CC1] |
| 6 | 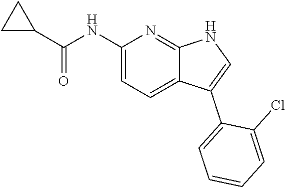 [SMILES: O=C(Nc1ccc2c(-c3ccccc3Cl)c[nH]c2n1)C1CC1] |
| 7 | 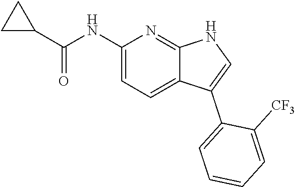 [SMILES: O=C(Nc1ccc2c(-c3ccccc3C(F)(F)F)c[nH]c2n1)C1CC1] |
| 8 | 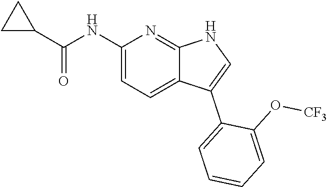 [SMILES: O=C(Nc1ccc2c(-c3ccccc3OC(F)(F)F)c[nH]c2n1)C1CC1] |
| 9 | 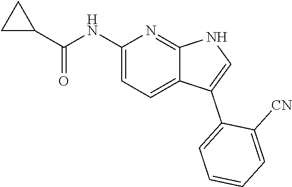 [SMILES: N#Cc1ccccc1-c1c[nH]c2nc(NC(=O)C3CC3)ccc12] |
| 10 | 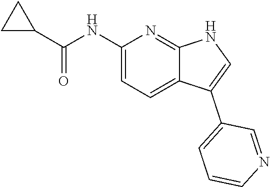 [SMILES: O=C(Nc1ccc2c(-c3cccnc3)c[nH]c2n1)C1CC1] |
| 11 | 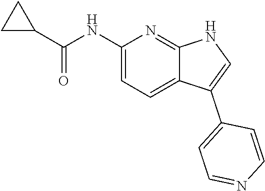 [SMILES: O=C(Nc1ccc2c(-c3ccncc3)c[nH]c2n1)C1CC1] |
| 12 | 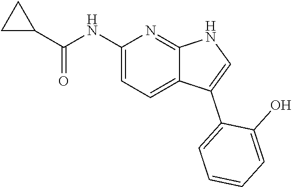 [SMILES: O=C(Nc1ccc2c(-c3ccccc3O)c[nH]c2n1)C1CC1] |
| 13 | 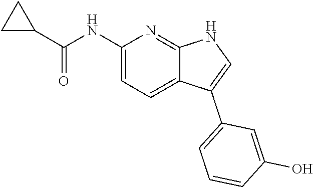 [SMILES: O=C(Nc1ccc2c(-c3cccc(O)c3)c[nH]c2n1)C1CC1] |
| 14 | 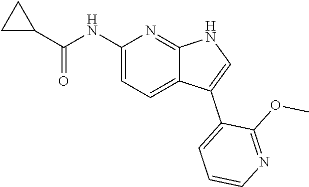 [SMILES: COc1ncccc1-c1c[nH]c2nc(NC(=O)C3CC3)ccc12] |
| 15 | 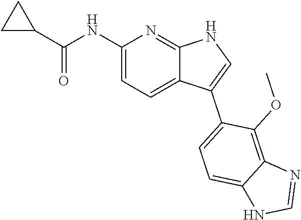 [SMILES: COc1c(-c2c[nH]c3nc(NC(=O)C4CC4)ccc23)ccc2[nH]cnc12] |
| 16 | 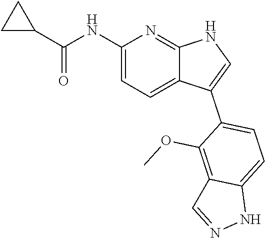 [SMILES: COc1c(-c2c[nH]c3nc(NC(=O)C4CC4)ccc23)ccc2[nH]ncc12] |
| 17 | 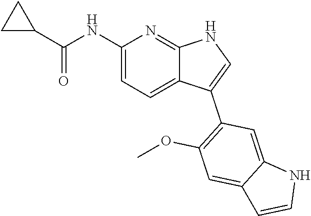 [SMILES: COc1cc2cc[nH]c2cc1-c1c[nH]c2nc(NC(=O)C3CC3)ccc12] |
| 18 | 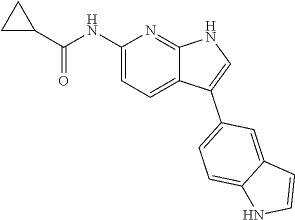 [SMILES: O=C(Nc1ccc2c(-c3ccc4[nH]ccc4c3)c[nH]c2n1)C1CC1] |
| 19 | 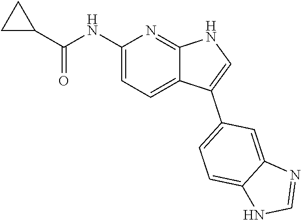 [SMILES: O=C(Nc1ccc2c(-c3ccc4[nH]cnc4c3)c[nH]c2n1)C1CC1] |
| 20 | 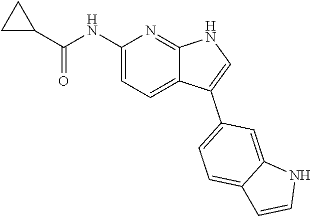 [SMILES: O=C(Nc1ccc2c(-c3ccc4cc[nH]c4c3)c[nH]c2n1)C1CC1] |
| 21 | 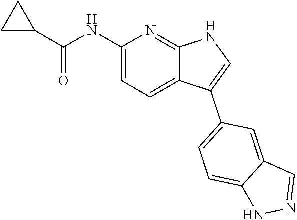 [SMILES: O=C(Nc1ccc2c(-c3ccc4[nH]ncc4c3)c[nH]c2n1)C1CC1] |
| 22 | 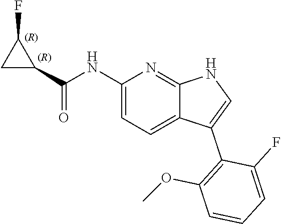 |
| 22-1 | 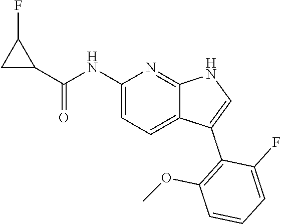 [SMILES: COc1cccc(F)c1-c1c[nH]c2nc(NC(=O)C3CC3F)ccc12] |
| 23 | 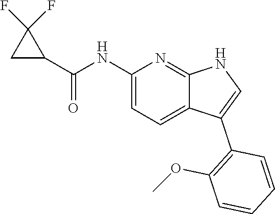 [SMILES: COc1ccccc1-c1c[nH]c2nc(NC(=O)C3CC3(F)F)ccc12] |
| 24 | 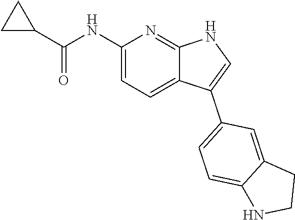 [SMILES: O=C(Nc1ccc2c(-c3ccc4c(c3)CCN4)c[nH]c2n1)C1CC1] |
| 25 | 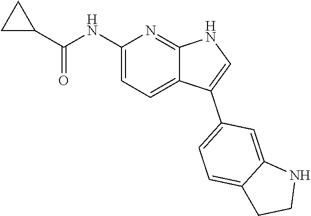 [SMILES: O=C(Nc1ccc2c(-c3ccc4c(c3)NCC4)c[nH]c2n1)C1CC1] |
| 26 | 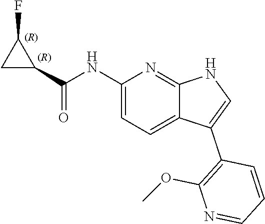 |
| 26-1 | 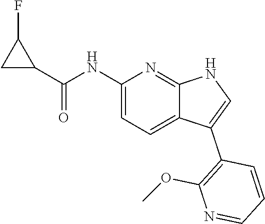 [SMILES: COc1ncccc1-c1c[nH]c2nc(NC(=O)C3CC3F)ccc12] |
| 27 | 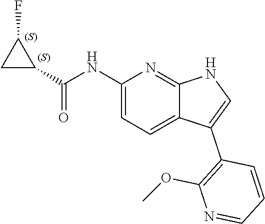 |
| 26-1 | 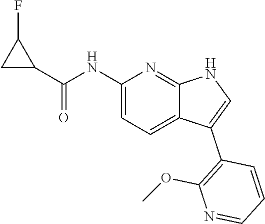 [SMILES: COc1ncccc1-c1c[nH]c2nc(NC(=O)C3CC3F)ccc12] |
| 28 | 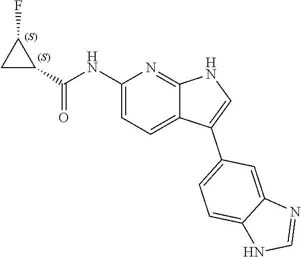 |
| 28-1 | 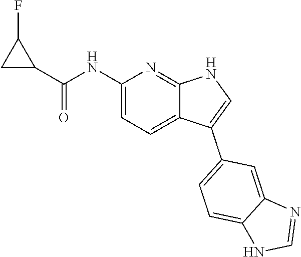 [SMILES: O=C(Nc1ccc2c(-c3ccc4[nH]cnc4c3)c[nH]c2n1)C1CC1F] |
| 29 | 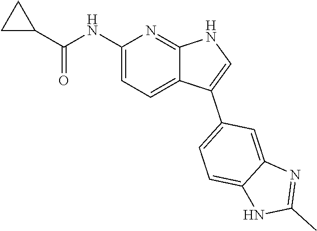 [SMILES: Cc1nc2cc(-c3c[nH]c4nc(NC(=O)C5CC5)ccc34)ccc2[nH]1] |
| 30 | 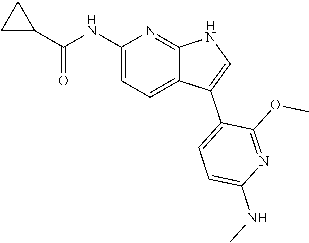 [SMILES: CNc1ccc(-c2c[nH]c3nc(NC(=O)C4CC4)ccc23)c(OC)n1] |
| 31 | 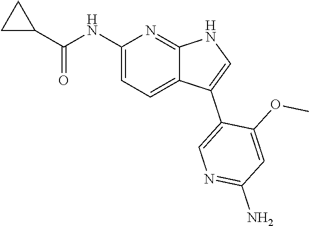 [SMILES: COc1cc(N)ncc1-c1c[nH]c2nc(NC(=O)C3CC3)ccc12] |
| 32 | 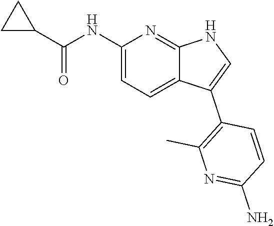 [SMILES: Cc1nc(N)ccc1-c1c[nH]c2nc(NC(=O)C3CC3)ccc12] |
| 33 | 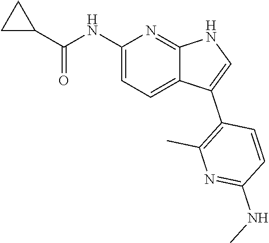 [SMILES: CNc1ccc(-c2c[nH]c3nc(NC(=O)C4CC4)ccc23)c(C)n1] |
| 34 | 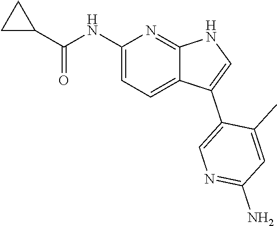 [SMILES: Cc1cc(N)ncc1-c1c[nH]c2nc(NC(=O)C3CC3)ccc12] |
| 35 | 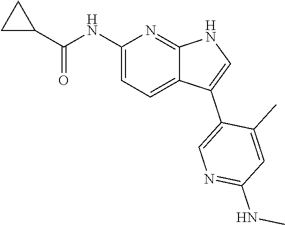 [SMILES: CNc1cc(C)c(-c2c[nH]c3nc(NC(=O)C4CC4)ccc23)cn1] |
| 36 | 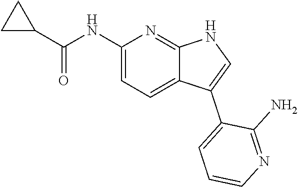 [SMILES: Nc1ncccc1-c1c[nH]c2nc(NC(=O)C3CC3)ccc12] |
| 37 | 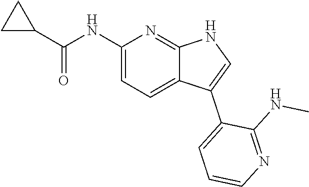 [SMILES: CNc1ncccc1-c1c[nH]c2nc(NC(=O)C3CC3)ccc12] |
| 38 | 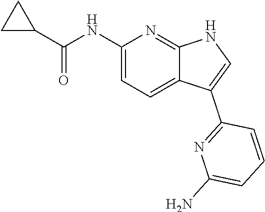 [SMILES: Nc1cccc(-c2c[nH]c3nc(NC(=O)C4CC4)ccc23)n1] |
| 39 | 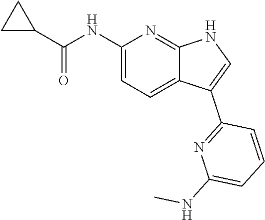 [SMILES: CNc1cccc(-c2c[nH]c3nc(NC(=O)C4CC4)ccc23)n1] |
| 40 | 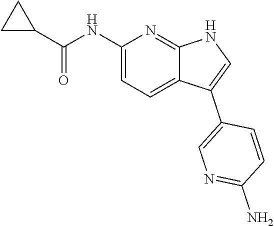 [SMILES: Nc1ccc(-c2c[nH]c3nc(NC(=O)C4CC4)ccc23)cn1] |
| 41 | 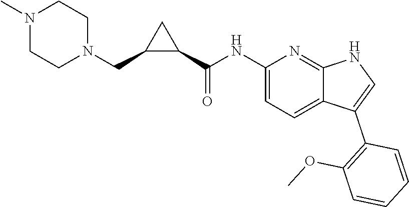 [SMILES: COc1ccccc1-c1c[nH]c2nc(NC(=O)[C@@H]3C[C@@H]3CN3CCN(C)CC3)ccc12] |
| 41-1 | 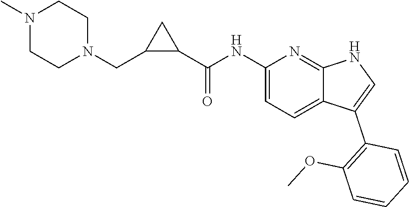 [SMILES: COc1ccccc1-c1c[nH]c2nc(NC(=O)C3CC3CN3CCN(C)CC3)ccc12] |
| 42 | 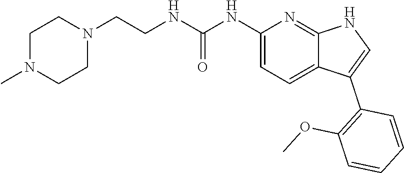 [SMILES: COc1ccccc1-c1c[nH]c2nc(NC(=O)NCCN3CCN(C)CC3)ccc12] |
| 43 | 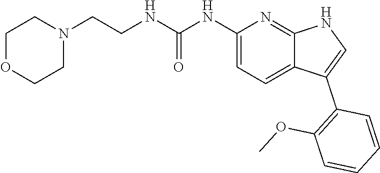 [SMILES: COc1ccccc1-c1c[nH]c2nc(NC(=O)NCCN3CCOCC3)ccc12] |
| 44 | 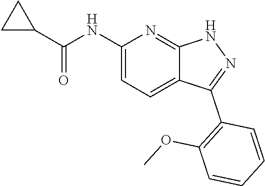 [SMILES: COc1ccccc1-c1n[nH]c2nc(NC(=O)C3CC3)ccc12] |
| 45 | 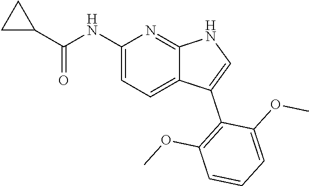 [SMILES: COc1cccc(OC)c1-c1c[nH]c2nc(NC(=O)C3CC3)ccc12] |
| 46 | 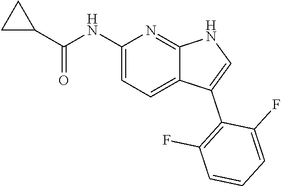 [SMILES: O=C(Nc1ccc2c(-c3c(F)cccc3F)c[nH]c2n1)C1CC1] |
| 47 | 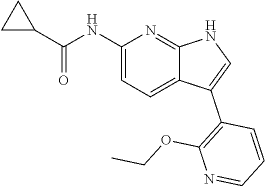 [SMILES: CCOc1ncccc1-c1c[nH]c2nc(NC(=O)C3CC3)ccc12] |
| 48 | 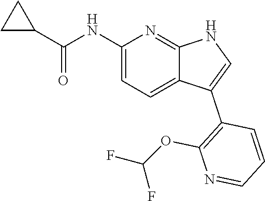 [SMILES: O=C(Nc1ccc2c(-c3cccnc3OC(F)F)c[nH]c2n1)C1CC1] |
| 49 | 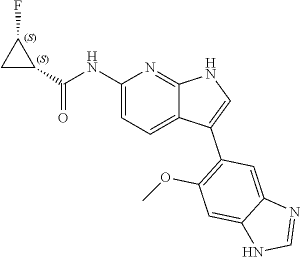 |
| 49-1 | 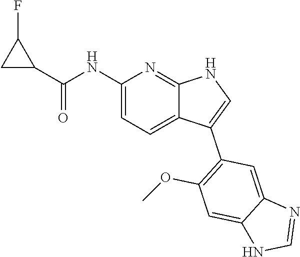 [SMILES: COc1cc2[nH]cnc2cc1-c1c[nH]c2nc(NC(=O)C3CC3F)ccc12] |
| 50 | 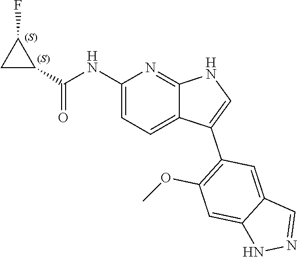 |
| 50-1 | 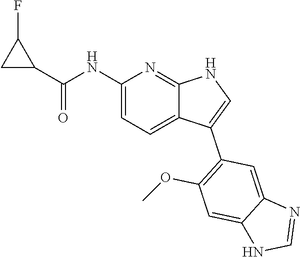 [SMILES: COc1cc2[nH]cnc2cc1-c1c[nH]c2nc(NC(=O)C3CC3F)ccc12] |
| 51 | 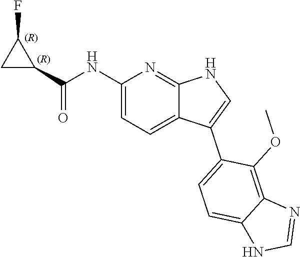 |
| 51-1 | 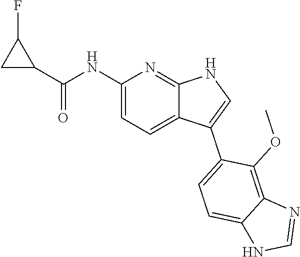 [SMILES: COc1c(-c2c[nH]c3nc(NC(=O)C4CC4F)ccc23)ccc2[nH]cnc12] |
| 52 | 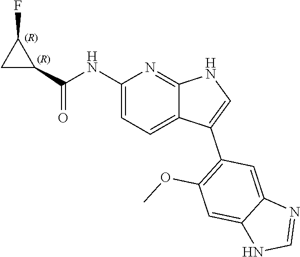 |
| 49-1 | 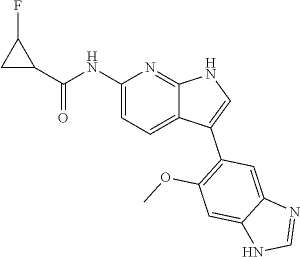 [SMILES: COc1cc2[nH]cnc2cc1-c1c[nH]c2nc(NC(=O)C3CC3F)ccc12] |
| 53 | 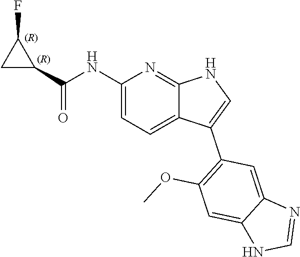 |
| 50-1 | 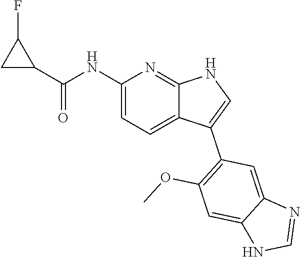 [SMILES: COc1cc2[nH]cnc2cc1-c1c[nH]c2nc(NC(=O)C3CC3F)ccc12] |
| 54 | 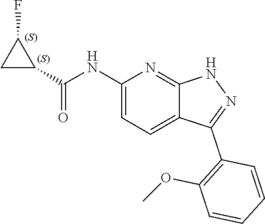 |
| 54-1 | 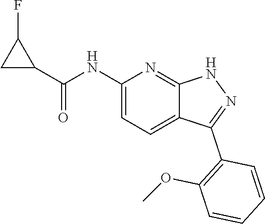 [SMILES: COc1ccccc1-c1n[nH]c2nc(NC(=O)C3CC3F)ccc12] |
| 55 | 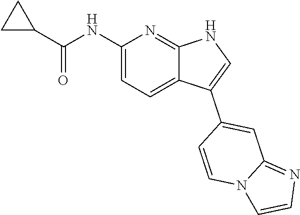 [SMILES: O=C(Nc1ccc2c(-c3ccn4ccnc4c3)c[nH]c2n1)C1CC1] |
| 56 | 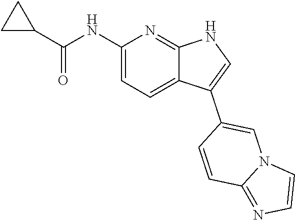 [SMILES: O=C(Nc1ccc2c(-c3ccc4nccn4c3)c[nH]c2n1)C1CC1] |
| 57 | 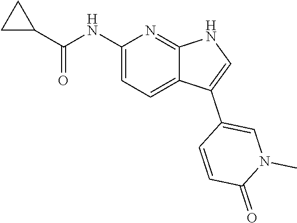 [SMILES: Cn1cc(-c2c[nH]c3nc(NC(=O)C4CC4)ccc23)ccc1=O] |
| 58 | 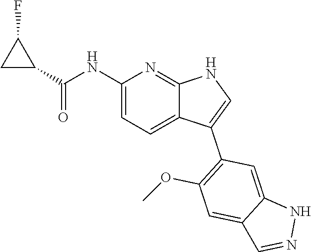 [SMILES: COc1cc2cn[nH]c2cc1-c1c[nH]c2nc(NC(=O)[C@@H]3C[C@@H]3F)ccc12] |
| 58-1 | 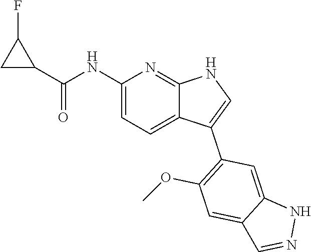 [SMILES: COc1cc2cn[nH]c2cc1-c1c[nH]c2nc(NC(=O)C3CC3F)ccc12] |
| 59 | 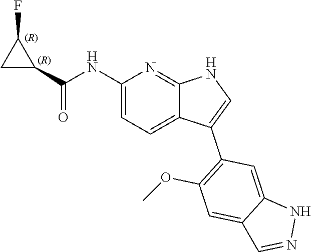 |
| 58-1 | 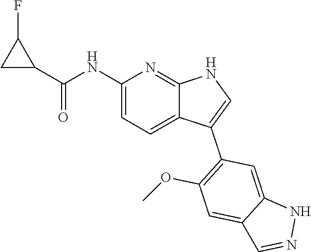 [SMILES: COc1cc2cn[nH]c2cc1-c1c[nH]c2nc(NC(=O)C3CC3F)ccc12] |
| 60 | 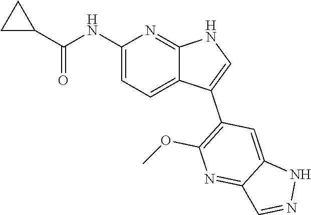 [SMILES: COc1nc2cn[nH]c2cc1-c1c[nH]c2nc(NC(=O)C3CC3)ccc12] |
| 61 | 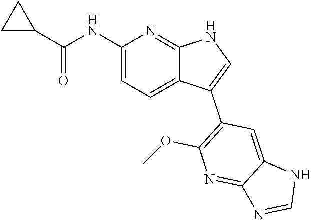 [SMILES: COc1nc2nc[nH]c2cc1-c1c[nH]c2nc(NC(=O)C3CC3)ccc12] |
| 62 | 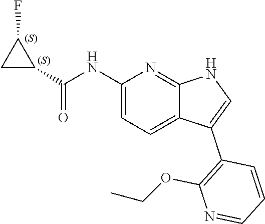 |
| 62-1 | 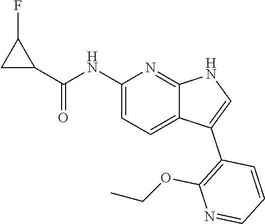 [SMILES: CCOc1ncccc1-c1c[nH]c2nc(NC(=O)C3CC3F)ccc12] |
| 63 | 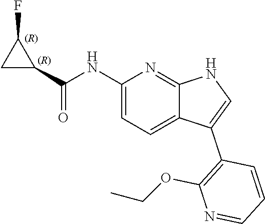 |
| 62-1 | 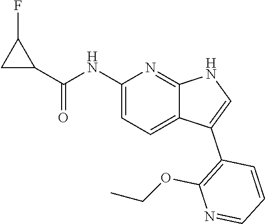 [SMILES: CCOc1ncccc1-c1c[nH]c2nc(NC(=O)C3CC3F)ccc12] |
| 64 | 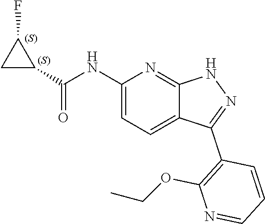 |
| 64-1 | 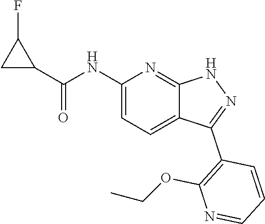 [SMILES: CCOc1ncccc1-c1n[nH]c2nc(NC(=O)C3CC3F)ccc12] |
| 65 | 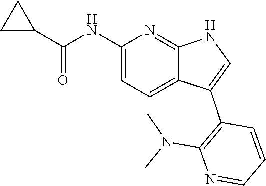 [SMILES: CN(C)c1ncccc1-c1c[nH]c2nc(NC(=O)C3CC3)ccc12] |
| 66 | 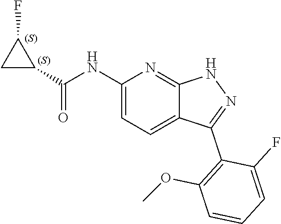 |
| 66-1 | 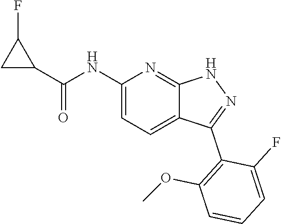 [SMILES: COc1cccc(F)c1-c1n[nH]c2nc(NC(=O)C3CC3F)ccc12] |
| 67 | 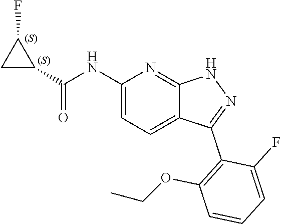 |
| 67-1 | 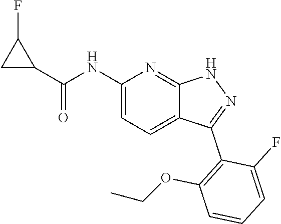 [SMILES: CCOc1cccc(F)c1-c1n[nH]c2nc(NC(=O)C3CC3F)ccc12] |
| 68 | 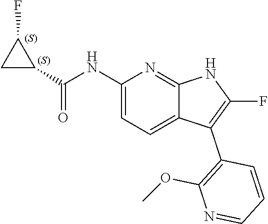 |
| 68-1 | 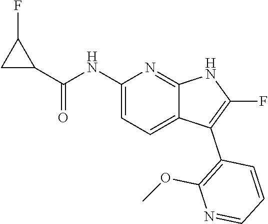 [SMILES: COc1ncccc1-c1c(F)[nH]c2nc(NC(=O)C3CC3F)ccc12] |
| 69 | 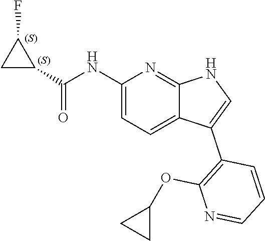 |
| 69-1 | 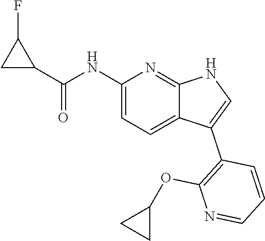 [SMILES: O=C(Nc1ccc2c(-c3cccnc3OC3CC3)c[nH]c2n1)C1CC1F] |
| 70 | 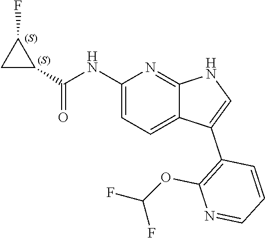 |
| 70-1 | 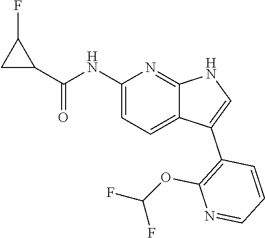 [SMILES: O=C(Nc1ccc2c(-c3cccnc3OC(F)F)c[nH]c2n1)C1CC1F] |
| 71 | 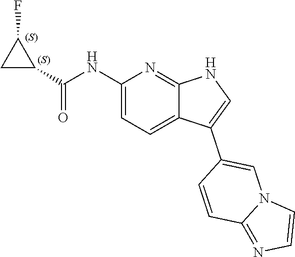 |
| 71-1 | 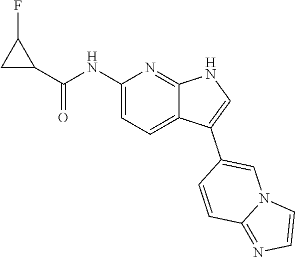 [SMILES: O=C(Nc1ccc2c(-c3ccc4nccn4c3)c[nH]c2n1)C1CC1F] |
| 72 | 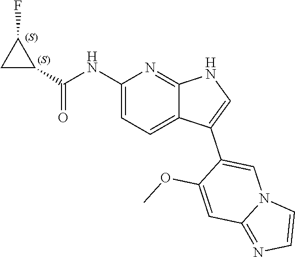 |
| 72-1 | 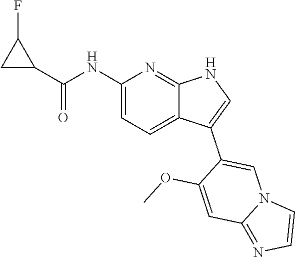 [SMILES: COc1cc2nccn2cc1-c1c[nH]c2nc(NC(=O)C3CC3F)ccc12] |
| 73 | 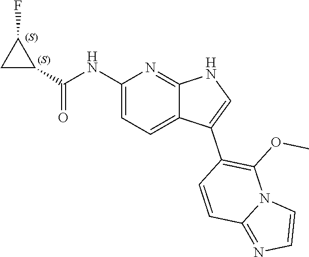 |
| 73-1 | 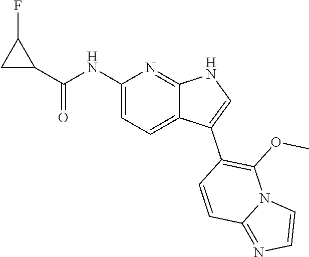 [SMILES: COc1c(-c2c[nH]c3nc(NC(=O)C4CC4F)ccc23)ccc2nccn12] |
| 74 | 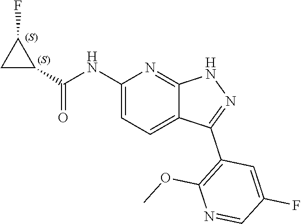 |
| 74-1 | 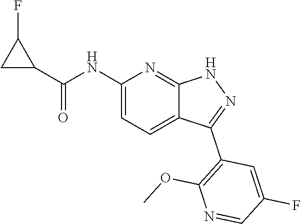 [SMILES: COc1ncc(F)cc1-c1n[nH]c2nc(NC(=O)C3CC3F)ccc12] |
| 75 | 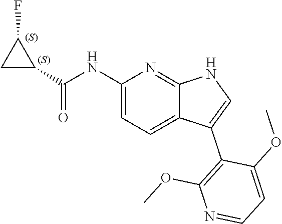 |
| 75-1 | 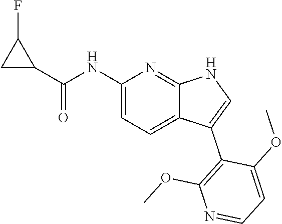 [SMILES: COc1ccnc(OC)c1-c1c[nH]c2nc(NC(=O)C3CC3F)ccc12] |
| 76 | 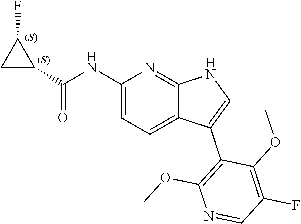 |
| 76-1 | 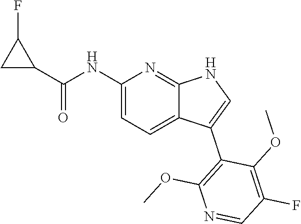 [SMILES: COc1ncc(F)c(OC)c1-c1c[nH]c2nc(NC(=O)C3CC3F)ccc12] |
| 77 | 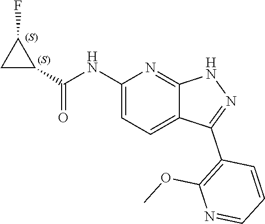 |
| 77-1 | 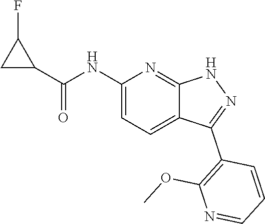 [SMILES: COc1ncccc1-c1n[nH]c2nc(NC(=O)C3CC3F)ccc12] |
| 78 | 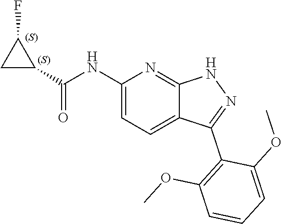 |
| 78-1 | 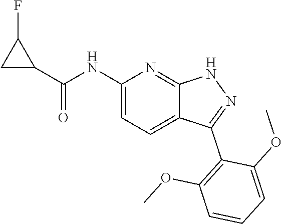 [SMILES: COc1cccc(OC)c1-c1n[nH]c2nc(NC(=O)C3CC3F)ccc12] |
| 79 | 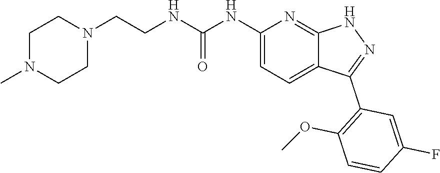 |
| 80 | 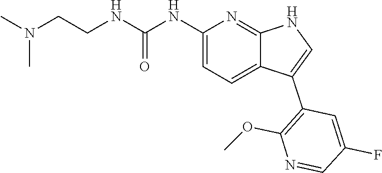 [SMILES: COc1ncc(F)cc1-c1c[nH]c2nc(NC(=O)NCCN(C)C)ccc12] |
| 81 | 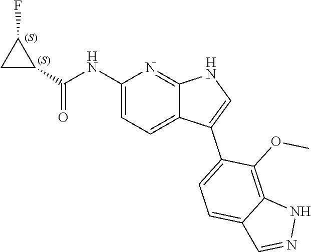 |
| 81-1 | 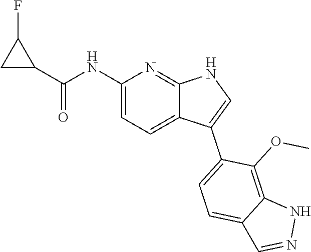 [SMILES: COc1c(-c2c[nH]c3nc(NC(=O)C4CC4F)ccc23)ccc2cn[nH]c12] |
| 82 | 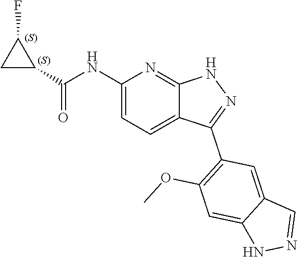 |
| 82-1 | 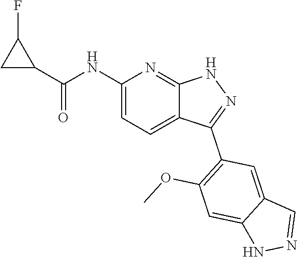 [SMILES: COc1cc2[nH]ncc2cc1-c1n[nH]c2nc(NC(=O)C3CC3F)ccc12] |
| 83 | 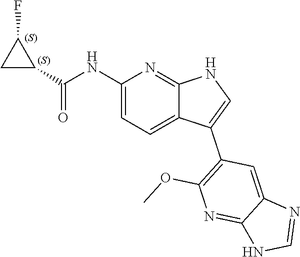 |
| 83-1 | 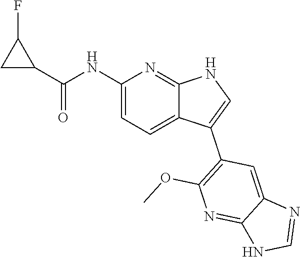 [SMILES: COc1nc2[nH]cnc2cc1-c1c[nH]c2nc(NC(=O)C3CC3F)ccc12] |
| 84 | 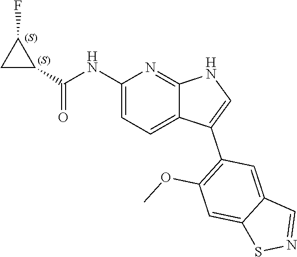 |
| 84-1 | 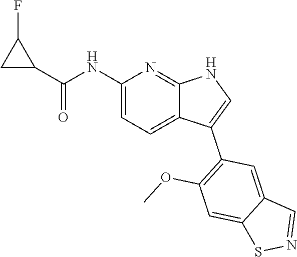 [SMILES: COc1cc2sncc2cc1-c1c[nH]c2nc(NC(=O)C3CC3F)ccc12] |
| 85 | 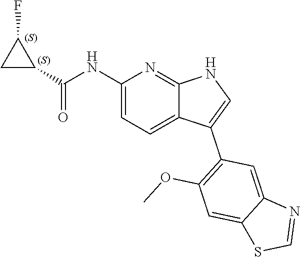 |
| 85-1 | 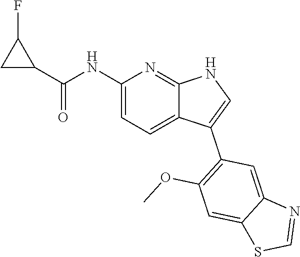 [SMILES: COc1cc2scnc2cc1-c1c[nH]c2nc(NC(=O)C3CC3F)ccc12] |
| 86 | 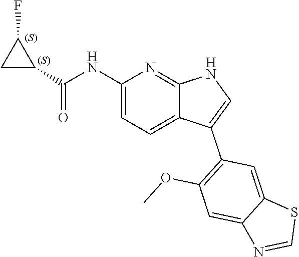 |
| 86-1 | 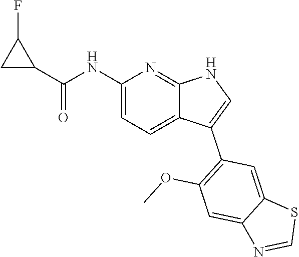 [SMILES: COc1cc2ncsc2cc1-c1c[nH]c2nc(NC(=O)C3CC3F)ccc12] |
| 87 | 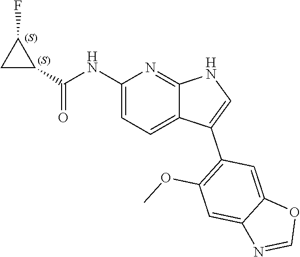 |
| 87-1 | 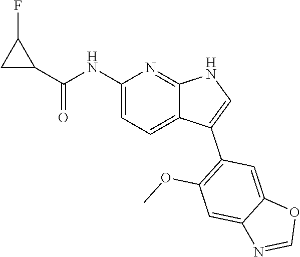 [SMILES: COc1cc2ncoc2cc1-c1c[nH]c2nc(NC(=O)C3CC3F)ccc12] |
| 88 | 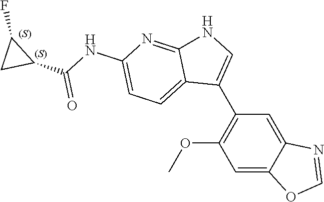 |
| 88-1 | 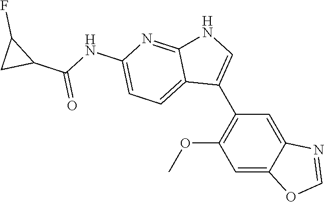 [SMILES: COc1cc2ocnc2cc1-c1c[nH]c2nc(NC(=O)C3CC3F)ccc12] |
| 89 | 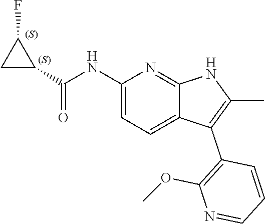 |
| 89-1 | 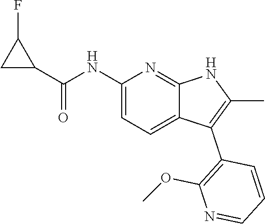 [SMILES: COc1ncccc1-c1c(C)[nH]c2nc(NC(=O)C3CC3F)ccc12] |
| 90 | 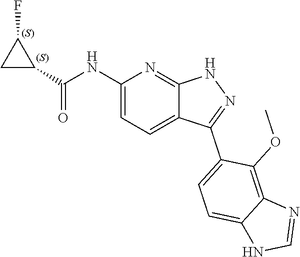 |
| 90-1 | 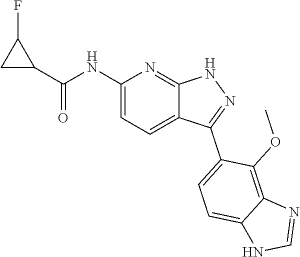 |
| 91 | 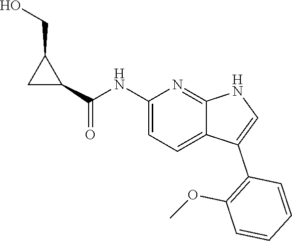 [SMILES: COc1ccccc1-c1c[nH]c2nc(NC(=O)[C@H]3C[C@H]3CO)ccc12] |
| 91-1 | 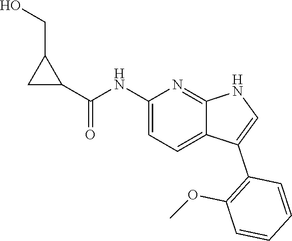 [SMILES: COc1ccccc1-c1c[nH]c2nc(NC(=O)C3CC3CO)ccc12] |
| 92 | 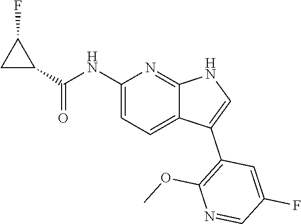 [SMILES: COc1ncc(F)cc1-c1c[nH]c2nc(NC(=O)[C@@H]3C[C@@H]3F)ccc12] |
| 92-1 | 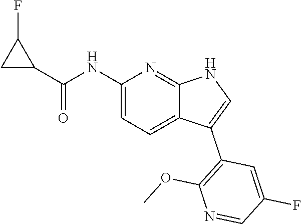 [SMILES: COc1ncc(F)cc1-c1c[nH]c2nc(NC(=O)C3CC3F)ccc12] |
| 93 | 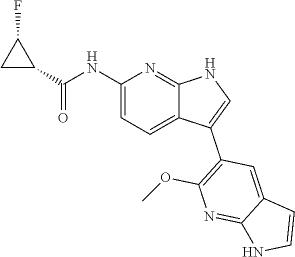 [SMILES: COc1nc2[nH]ccc2cc1-c1c[nH]c2nc(NC(=O)[C@@H]3C[C@@H]3F)ccc12] |
| 93-1 | 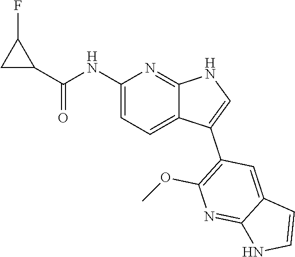 [SMILES: COc1nc2[nH]ccc2cc1-c1c[nH]c2nc(NC(=O)C3CC3F)ccc12] |
| 94 | 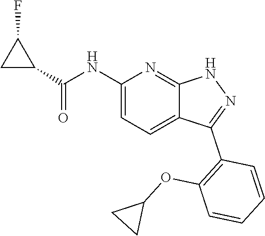 [SMILES: O=C(Nc1ccc2c(-c3ccccc3OC3CC3)n[nH]c2n1)[C@@H]1C[C@@H]1F] |
| 94-1 | 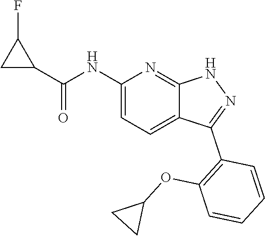 [SMILES: O=C(Nc1ccc2c(-c3ccccc3OC3CC3)n[nH]c2n1)C1CC1F] |
| 95 | 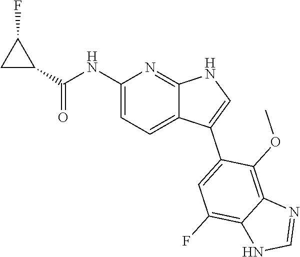 [SMILES: COc1c(-c2c[nH]c3nc(NC(=O)[C@@H]4C[C@@H]4F)ccc23)cc(F)c2[nH]cnc12] |
| 95-1 | 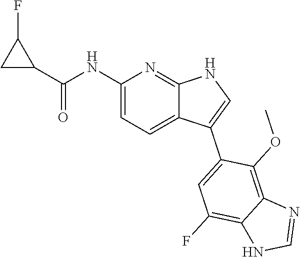 [SMILES: COc1c(-c2c[nH]c3nc(NC(=O)C4CC4F)ccc23)cc(F)c2[nH]cnc12] |
| 96 | 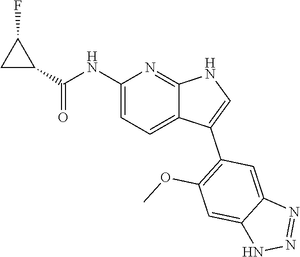 [SMILES: COc1cc2[nH]nnc2cc1-c1c[nH]c2nc(NC(=O)[C@@H]3C[C@@H]3F)ccc12] |
| 96-1 | 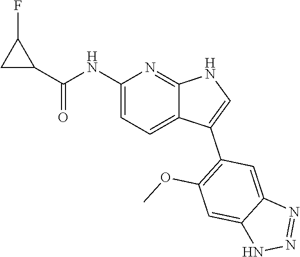 [SMILES: COc1cc2[nH]nnc2cc1-c1c[nH]c2nc(NC(=O)C3CC3F)ccc12] |
| 97 | 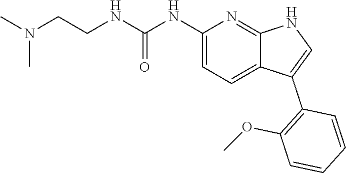 [SMILES: COc1ccccc1-c1c[nH]c2nc(NC(=O)NCCN(C)C)ccc12] |
| 98 | 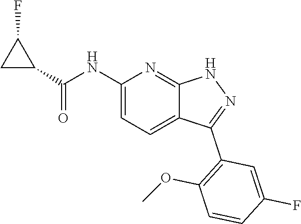 [SMILES: COc1ccc(F)cc1-c1n[nH]c2nc(NC(=O)[C@@H]3C[C@@H]3F)ccc12] |
| 98-1 | 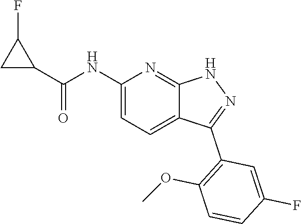 [SMILES: COc1ccc(F)cc1-c1n[nH]c2nc(NC(=O)C3CC3F)ccc12] |
| 99 | 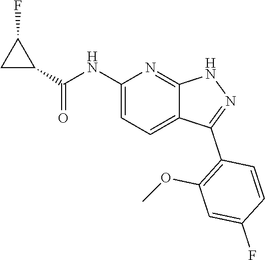 [SMILES: COc1cc(F)ccc1-c1n[nH]c2nc(NC(=O)[C@@H]3C[C@@H]3F)ccc12] |
| 99-1 | 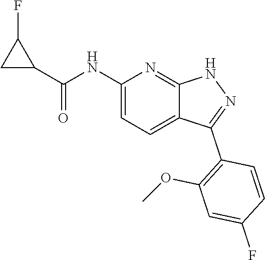 [SMILES: COc1cc(F)ccc1-c1n[nH]c2nc(NC(=O)C3CC3F)ccc12] |
| 100 | 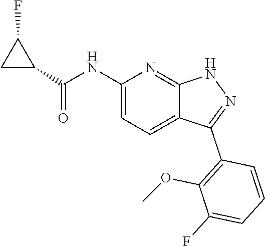 [SMILES: COc1c(F)cccc1-c1n[nH]c2nc(NC(=O)[C@@H]3C[C@@H]3F)ccc12] |
| 100-1 | 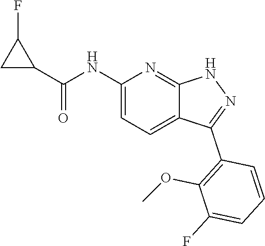 [SMILES: COc1c(F)cccc1-c1n[nH]c2nc(NC(=O)C3CC3F)ccc12] |
| 101 | 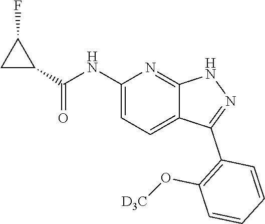 [SMILES: [2H]C([2H])([2H])Oc1ccccc1-c1n[nH]c2nc(NC(=O)[C@@H]3C[C@@H]3F)ccc12] |
| 101-1 | 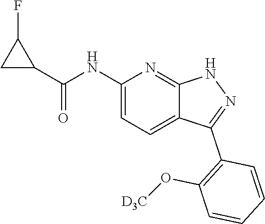 [SMILES: [2H]C([2H])([2H])Oc1ccccc1-c1n[nH]c2nc(NC(=O)C3CC3F)ccc12] |
| 102 | 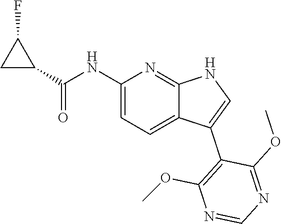 [SMILES: COc1ncnc(OC)c1-c1c[nH]c2nc(NC(=O)[C@@H]3C[C@@H]3F)ccc12] |
| 102-1 | 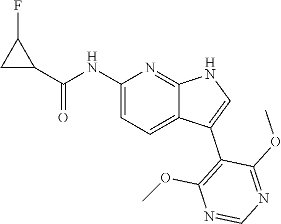 [SMILES: COc1ncnc(OC)c1-c1c[nH]c2nc(NC(=O)C3CC3F)ccc12] |
| 103 | 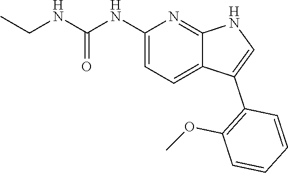 [SMILES: CCNC(=O)Nc1ccc2c(-c3ccccc3OC)c[nH]c2n1] |
| 104 | 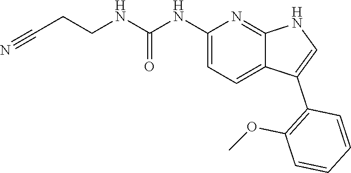 [SMILES: COc1ccccc1-c1c[nH]c2nc(NC(=O)NCCC#N)ccc12] |
| 105 | 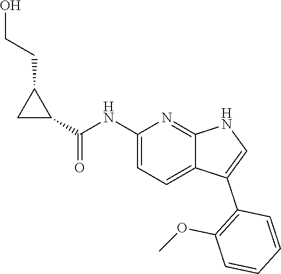 [SMILES: COc1ccccc1-c1c[nH]c2nc(NC(=O)[C@@H]3C[C@@H]3CCO)ccc12] |
| 105-1 | 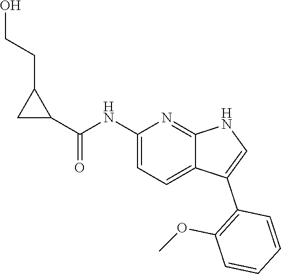 [SMILES: COc1ccccc1-c1c[nH]c2nc(NC(=O)C3CC3CCO)ccc12] |
| 106 | 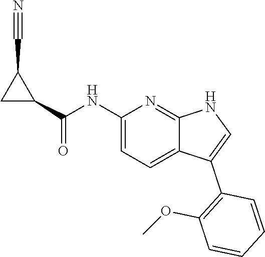 [SMILES: COc1ccccc1-c1c[nH]c2nc(NC(=O)[C@H]3C[C@H]3C#N)ccc12] |
| 106-1 | 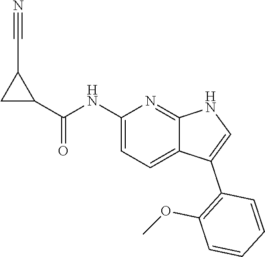 [SMILES: COc1ccccc1-c1c[nH]c2nc(NC(=O)C3CC3C#N)ccc12] |
| 107 | 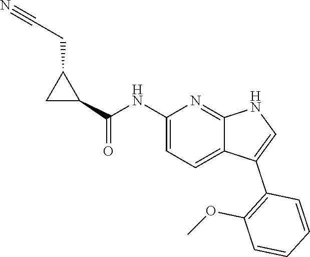 [SMILES: COc1ccccc1-c1c[nH]c2nc(NC(=O)[C@H]3C[C@@H]3CC#N)ccc12] |
| 107-1 | 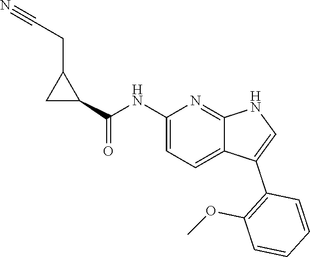 [SMILES: COc1ccccc1-c1c[nH]c2nc(NC(=O)[C@H]3CC3CC#N)ccc12] |
| 108 | 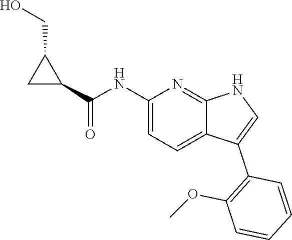 [SMILES: COc1ccccc1-c1c[nH]c2nc(NC(=O)[C@H]3C[C@@H]3CO)ccc12] |
| 108-1 | 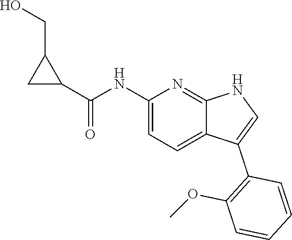 [SMILES: COc1ccccc1-c1c[nH]c2nc(NC(=O)C3CC3CO)ccc12] |
| 109 | 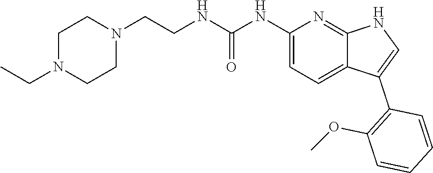 [SMILES: CCN1CCN(CCNC(=O)Nc2ccc3c(-c4ccccc4OC)c[nH]c3n2)CC1] |
| 110 | 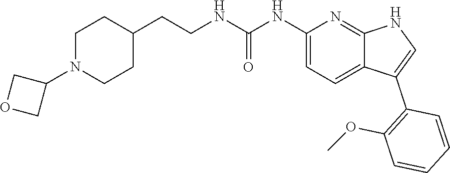 [SMILES: COc1ccccc1-c1c[nH]c2nc(NC(=O)NCCC3CCN(C4COC4)CC3)ccc12] |
| 111 | 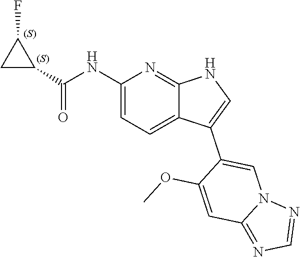 |
| 111-1 | 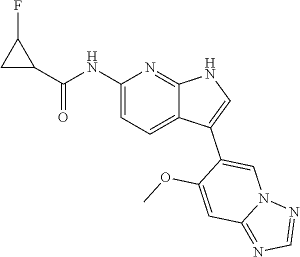 [SMILES: COc1cc2ncnn2cc1-c1c[nH]c2nc(NC(=O)C3CC3F)ccc12] |
| 112 | 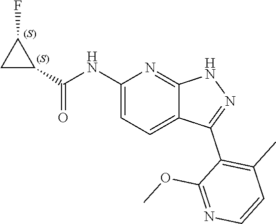 |
| 112-1 | 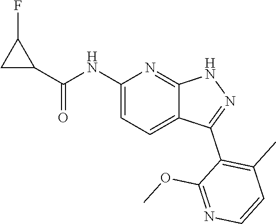 [SMILES: COc1nccc(C)c1-c1n[nH]c2nc(NC(=O)C3CC3F)ccc12] |
| 113 | 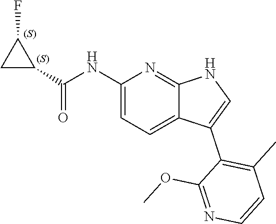 |
| 113-1 | 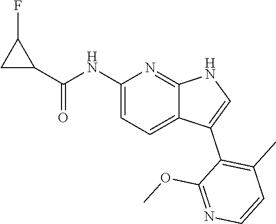 [SMILES: COc1nccc(C)c1-c1c[nH]c2nc(NC(=O)C3CC3F)ccc12] |
| 114 | 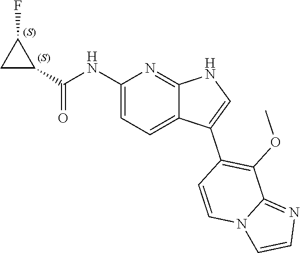 |
| 114-1 | 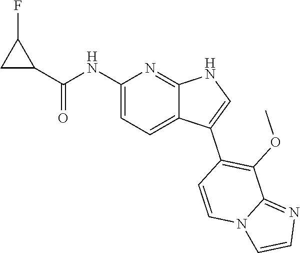 [SMILES: COc1c(-c2c[nH]c3nc(NC(=O)C4CC4F)ccc23)ccn2ccnc12] |
| 115 | 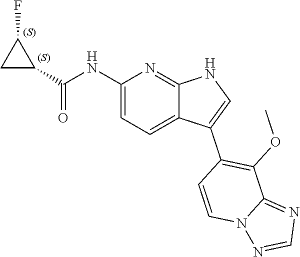 |
| 115-1 | 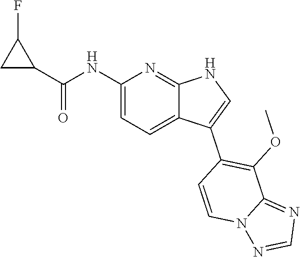 [SMILES: COc1c(-c2c[nH]c3nc(NC(=O)C4CC4F)ccc23)ccn2ncnc12] |
| 116 | 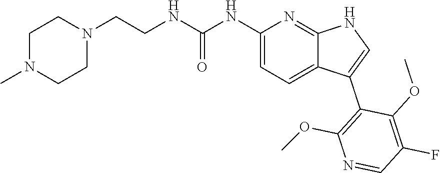 [SMILES: COc1ncc(F)c(OC)c1-c1c[nH]c2nc(NC(=O)NCCN3CCN(C)CC3)ccc12] |
| 117 | 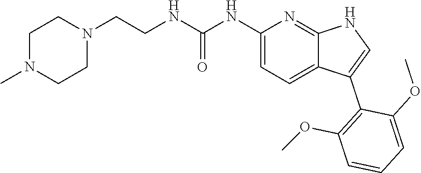 [SMILES: COc1cccc(OC)c1-c1c[nH]c2nc(NC(=O)NCCN3CCN(C)CC3)ccc12] |
| 118 | 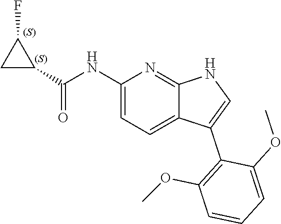 |
| 118-1 | 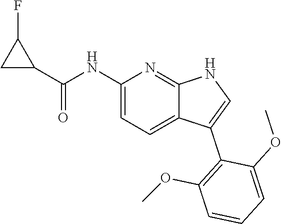 [SMILES: COc1cccc(OC)c1-c1c[nH]c2nc(NC(=O)C3CC3F)ccc12] |
| 119 | 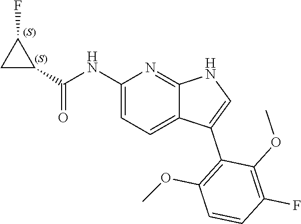 |
| 119-1 | 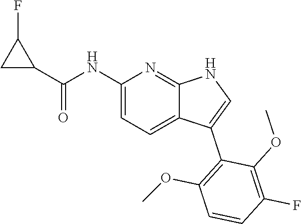 [SMILES: COc1ccc(F)c(OC)c1-c1c[nH]c2nc(NC(=O)C3CC3F)ccc12] |
| 120 | 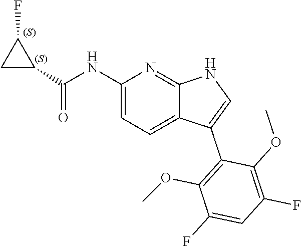 |
| 120-1 | 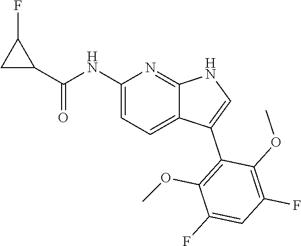 [SMILES: COc1c(F)cc(F)c(OC)c1-c1c[nH]c2nc(NC(=O)C3CC3F)ccc12] |
| 121 | 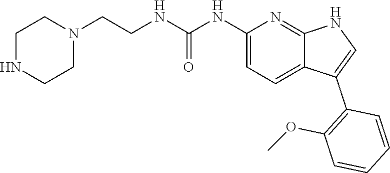 [SMILES: COc1ccccc1-c1c[nH]c2nc(NC(=O)NCCN3CCNCC3)ccc12] |
| 122 | 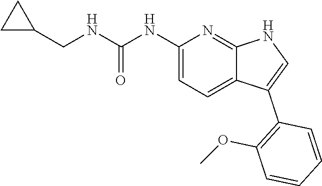 [SMILES: COc1ccccc1-c1c[nH]c2nc(NC(=O)NCC3CC3)ccc12] |
| 123 | 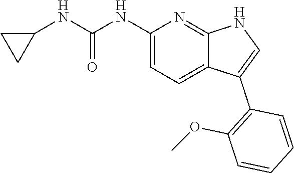 [SMILES: COc1ccccc1-c1c[nH]c2nc(NC(=O)NC3CC3)ccc12] |
| 124 | 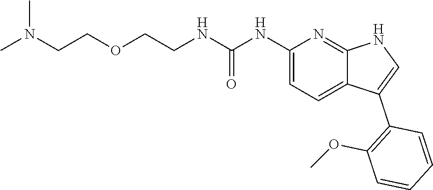 [SMILES: COc1ccccc1-c1c[nH]c2nc(NC(=O)NCCOCCN(C)C)ccc12] |
| 125 | 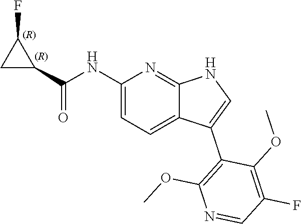 |
| 125-1 | 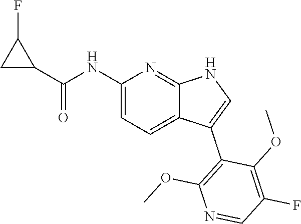 [SMILES: COc1ncc(F)c(OC)c1-c1c[nH]c2nc(NC(=O)C3CC3F)ccc12] |
| 126 | 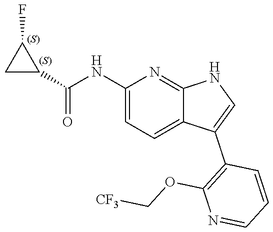 |
| 126-1 | 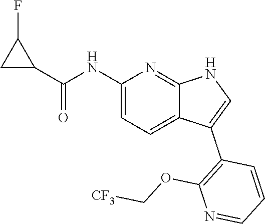 [SMILES: O=C(Nc1ccc2c(-c3cccnc3OCC(F)(F)F)c[nH]c2n1)C1CC1F] |
| 127 | 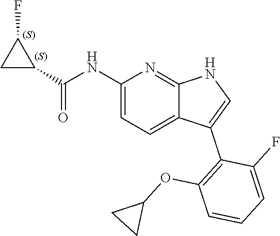 |
| 127-1 | 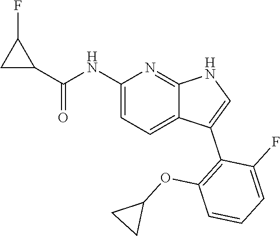 [SMILES: O=C(Nc1ccc2c(-c3c(F)cccc3OC3CC3)c[nH]c2n1)C1CC1F] |
| 128 | 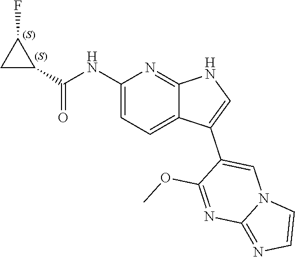 |
| 128-1 | 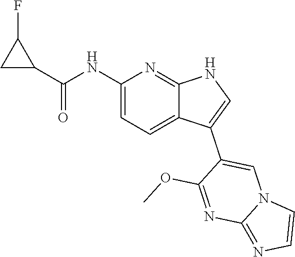 [SMILES: COc1nc2nccn2cc1-c1c[nH]c2nc(NC(=O)C3CC3F)ccc12] |
| 129 | 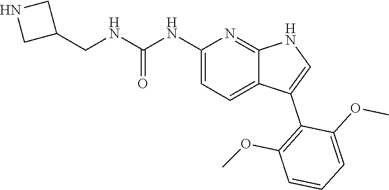 [SMILES: COc1cccc(OC)c1-c1c[nH]c2nc(NC(=O)NCC3CNC3)ccc12] |
| 130 | 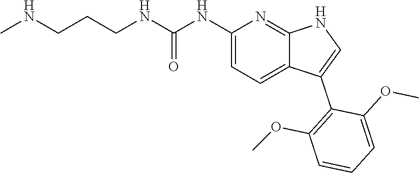 |
| 131 | 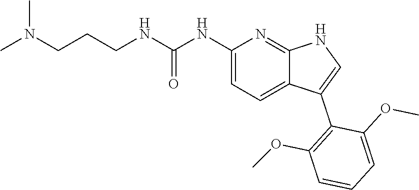 |
| 132 | 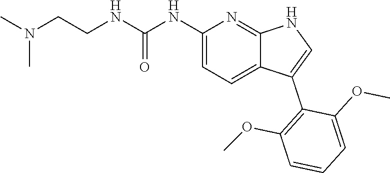 |
| 133 | 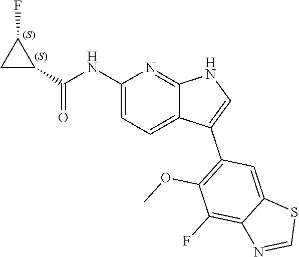 |
| 133-1 | 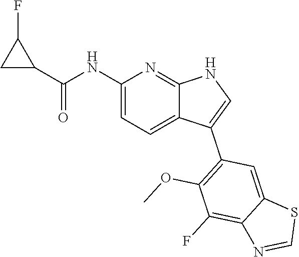 [SMILES: COc1c(-c2c[nH]c3nc(NC(=O)C4CC4F)ccc23)cc2scnc2c1F] |
| 134 | 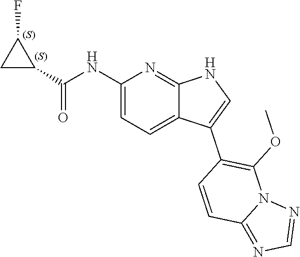 |
| 134-1 | 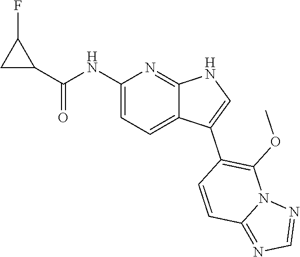 [SMILES: COc1c(-c2c[nH]c3nc(NC(=O)C4CC4F)ccc23)ccc2ncnn12] |
| 135 | 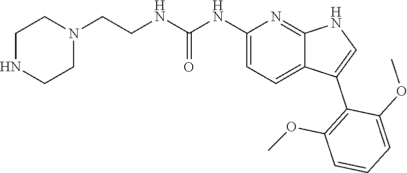 [SMILES: COc1cccc(OC)c1-c1c[nH]c2nc(NC(=O)NCCN3CCNCC3)ccc12] |
| 136 | 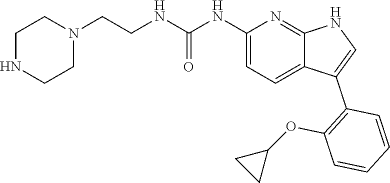 [SMILES: O=C(NCCN1CCNCC1)Nc1ccc2c(-c3ccccc3OC3CC3)c[nH]c2n1] |
| 137 | 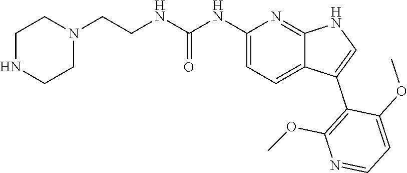 [SMILES: COc1ccnc(OC)c1-c1c[nH]c2nc(NC(=O)NCCN3CCNCC3)ccc12] |
| 138 | 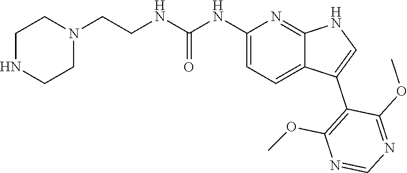 [SMILES: COc1ncnc(OC)c1-c1c[nH]c2nc(NC(=O)NCCN3CCNCC3)ccc12] |
| 139 | 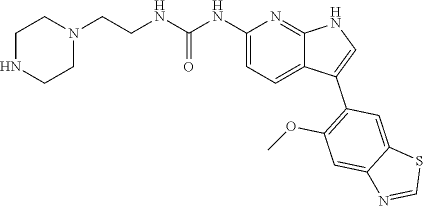 [SMILES: COc1cc2ncsc2cc1-c1c[nH]c2nc(NC(=O)NCCN3CCNCC3)ccc12] |
| 140 | 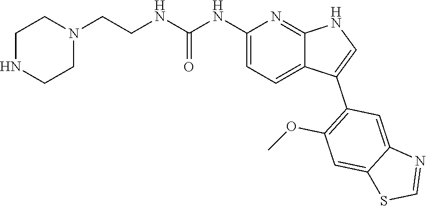 [SMILES: COc1cc2scnc2cc1-c1c[nH]c2nc(NC(=O)NCCN3CCNCC3)ccc12] |
| 141 | 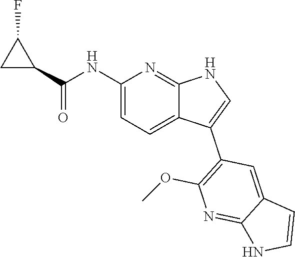 [SMILES: COc1nc2[nH]ccc2cc1-c1c[nH]c2nc(NC(=O)[C@H]3C[C@@H]3F)ccc12] |
| 141-1 | 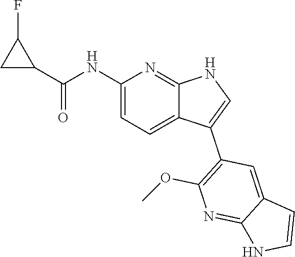 [SMILES: COc1nc2[nH]ccc2cc1-c1c[nH]c2nc(NC(=O)C3CC3F)ccc12] |
| 142 | 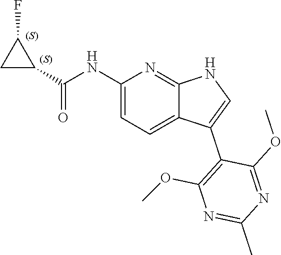 |
| 142-1 | 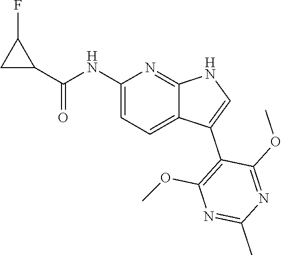 [SMILES: COc1nc(C)nc(OC)c1-c1c[nH]c2nc(NC(=O)C3CC3F)ccc12] |
| 143 | 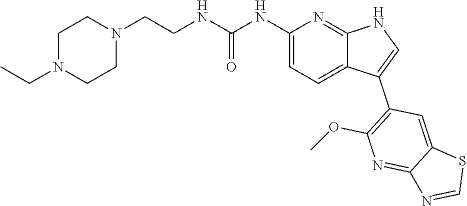 [SMILES: CCN1CCN(CCNC(=O)Nc2ccc3c(-c4cc5scnc5nc4OC)c[nH]c3n2)CC1] |
| 144 | 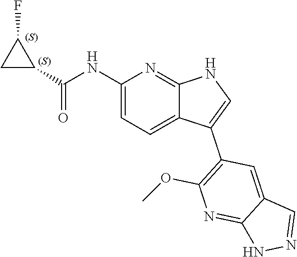 |
| 144-1 | 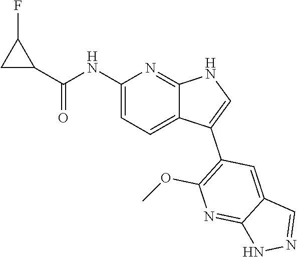 [SMILES: COc1nc2[nH]ncc2cc1-c1c[nH]c2nc(NC(=O)C3CC3F)ccc12] |
| 145 | 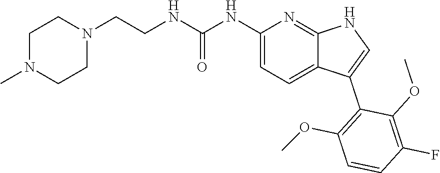 [SMILES: COc1ccc(F)c(OC)c1-c1c[nH]c2nc(NC(=O)NCCN3CCN(C)CC3)ccc12] |
| 146 | 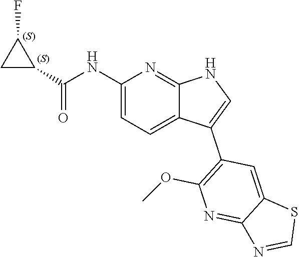 |
| 146-1 | 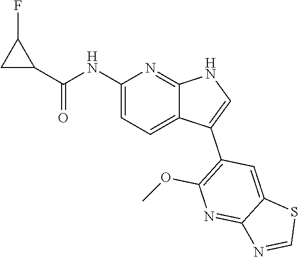 [SMILES: COc1nc2ncsc2cc1-c1c[nH]c2nc(NC(=O)C3CC3F)ccc12] |
| 147 | 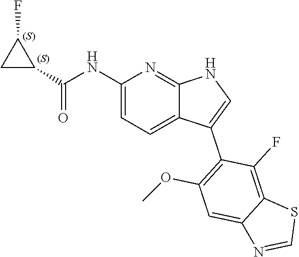 |
| 147-1 | 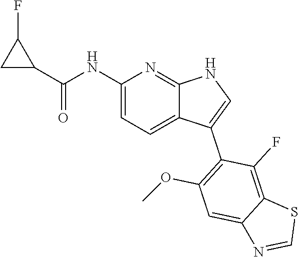 [SMILES: COc1cc2ncsc2c(F)c1-c1c[nH]c2nc(NC(=O)C3CC3F)ccc12] |
| 148 | 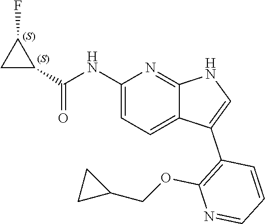 |
| 148-1 | 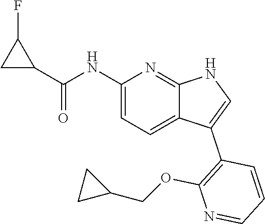 [SMILES: O=C(Nc1ccc2c(-c3cccnc3OCC3CC3)c[nH]c2n1)C1CC1F] |
| 149 | 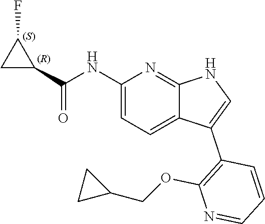 |
| 148-1 | 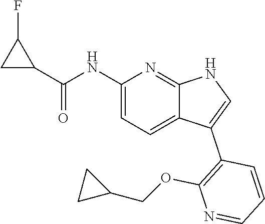 [SMILES: O=C(Nc1ccc2c(-c3cccnc3OCC3CC3)c[nH]c2n1)C1CC1F] |
| 149 | 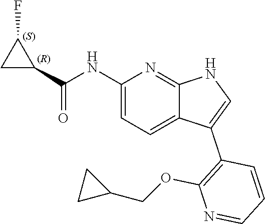 |
| 148-1 | 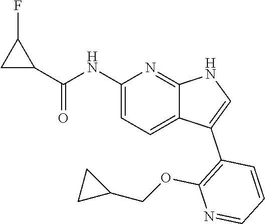 [SMILES: O=C(Nc1ccc2c(-c3cccnc3OCC3CC3)c[nH]c2n1)C1CC1F] |
| 150 | 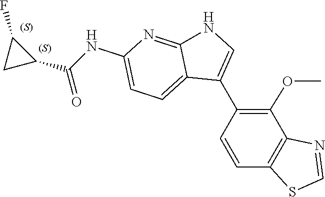 |
| 150-1 | 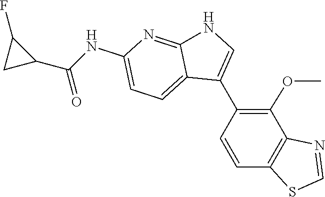 [SMILES: COc1c(-c2c[nH]c3nc(NC(=O)C4CC4F)ccc23)ccc2scnc12] |
| 151 | 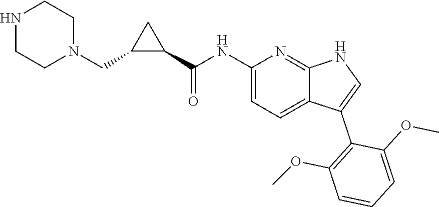 [SMILES: COc1cccc(OC)c1-c1c[nH]c2nc(NC(=O)[C@@H]3C[C@H]3CN3CCNCC3)ccc12] |
| 151-1 | 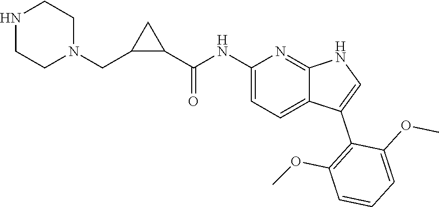 [SMILES: COc1cccc(OC)c1-c1c[nH]c2nc(NC(=O)C3CC3CN3CCNCC3)ccc12] |
| 152 | 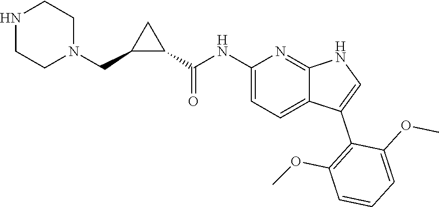 [SMILES: COc1cccc(OC)c1-c1c[nH]c2nc(NC(=O)[C@H]3C[C@@H]3CN3CCNCC3)ccc12] |
| 151-1 | 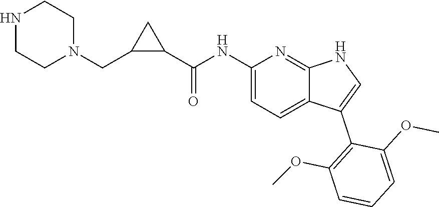 [SMILES: COc1cccc(OC)c1-c1c[nH]c2nc(NC(=O)C3CC3CN3CCNCC3)ccc12] |
| 153 | 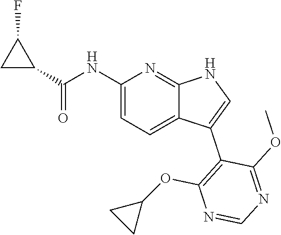 [SMILES: COc1ncnc(OC2CC2)c1-c1c[nH]c2nc(NC(=O)[C@@H]3C[C@@H]3F)ccc12] |
| 153-1 | 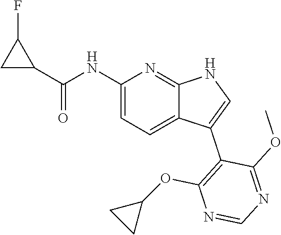 [SMILES: COc1ncnc(OC2CC2)c1-c1c[nH]c2nc(NC(=O)C3CC3F)ccc12] |
| 154 | 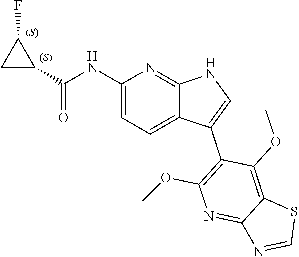 |
| 154-1 | 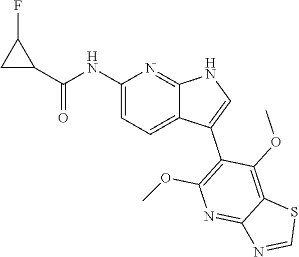 [SMILES: COc1nc2ncsc2c(OC)c1-c1c[nH]c2nc(NC(=O)C3CC3F)ccc12] |
| 155 | 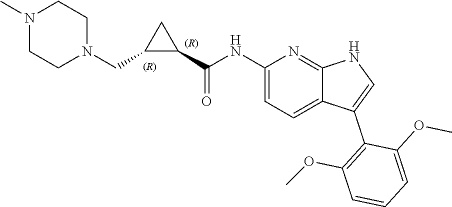 |
| 155-1 | 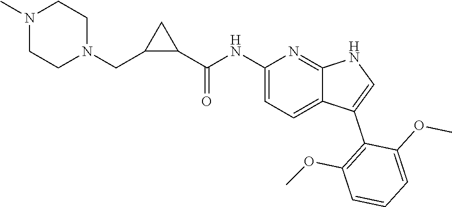 |
| 156 | 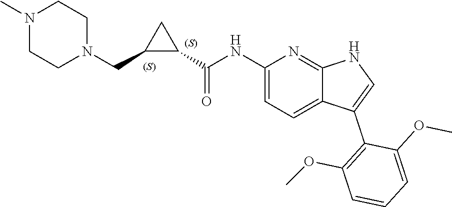 |
| 155-1 | 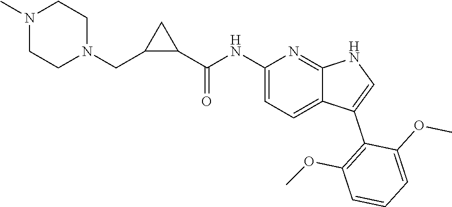 |
| 157 | 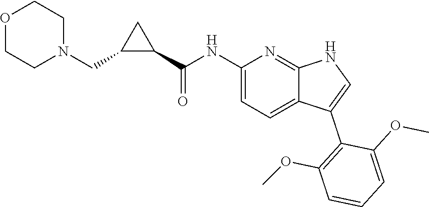 [SMILES: COc1cccc(OC)c1-c1c[nH]c2nc(NC(=O)[C@@H]3C[C@H]3CN3CCOCC3)ccc12] |
| 157-1 | 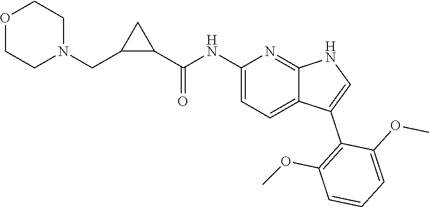 [SMILES: COc1cccc(OC)c1-c1c[nH]c2nc(NC(=O)C3CC3CN3CCOCC3)ccc12] |
| 158 | 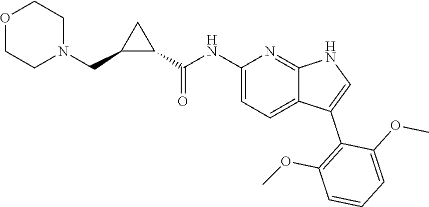 [SMILES: COc1cccc(OC)c1-c1c[nH]c2nc(NC(=O)[C@H]3C[C@@H]3CN3CCOCC3)ccc12] |
| 157-1 | 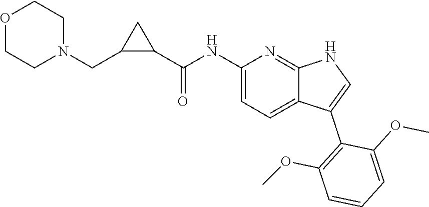 [SMILES: COc1cccc(OC)c1-c1c[nH]c2nc(NC(=O)C3CC3CN3CCOCC3)ccc12] |
| 159 | 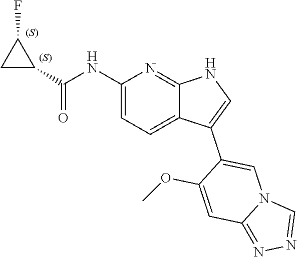 |
| 159-1 | 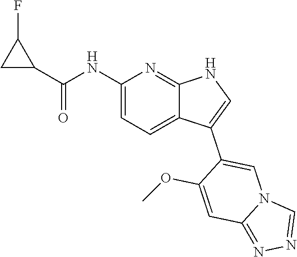 [SMILES: COc1cc2nncn2cc1-c1c[nH]c2nc(NC(=O)C3CC3F)ccc12] |
| 160 | 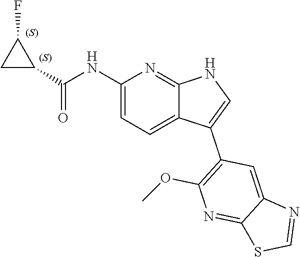 |
| 160-1 | 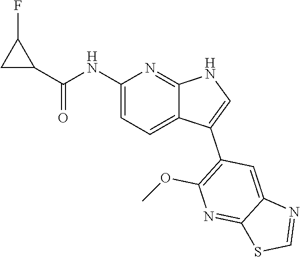 [SMILES: COc1nc2scnc2cc1-c1c[nH]c2nc(NC(=O)C3CC3F)ccc12] |
| 161 | 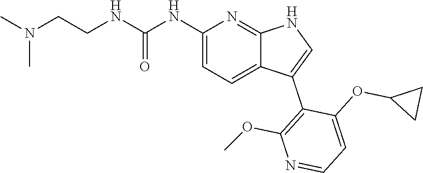 [SMILES: COc1nccc(OC2CC2)c1-c1c[nH]c2nc(NC(=O)NCCN(C)C)ccc12] |
| 162 | 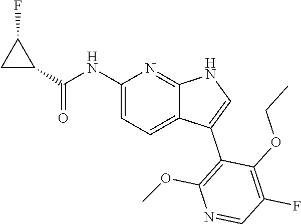 [SMILES: CCOc1c(F)cnc(OC)c1-c1c[nH]c2nc(NC(=O)[C@@H]3C[C@@H]3F)ccc12] |
| 162-1 | 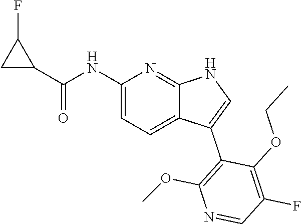 [SMILES: CCOc1c(F)cnc(OC)c1-c1c[nH]c2nc(NC(=O)C3CC3F)ccc12] |
| 163 | 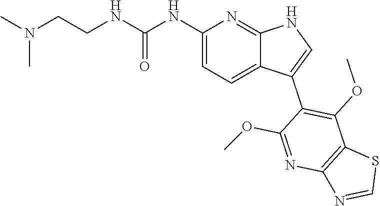 [SMILES: COc1nc2ncsc2c(OC)c1-c1c[nH]c2nc(NC(=O)NCCN(C)C)ccc12] |
| 164 | 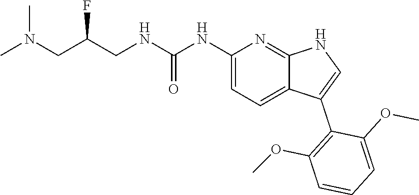 |
| 164-1 | 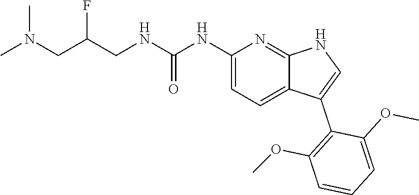 |
| 165 | 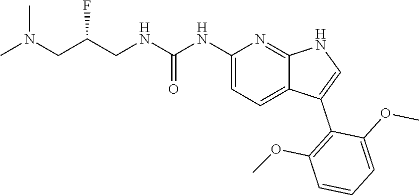 |
| 164-1 | 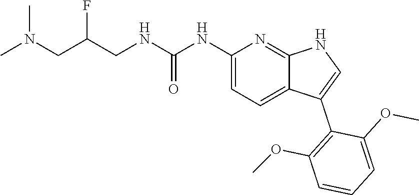 |
| 166 | 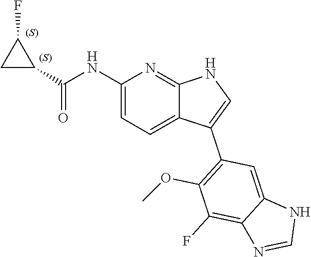 |
| 166-1 | 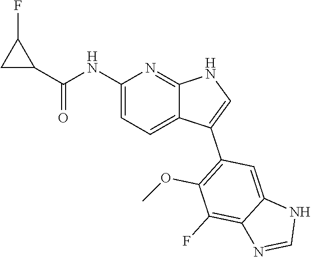 [SMILES: COc1c(-c2c[nH]c3nc(NC(=O)C4CC4F)ccc23)cc2[nH]cnc2c1F] |
| 167 | 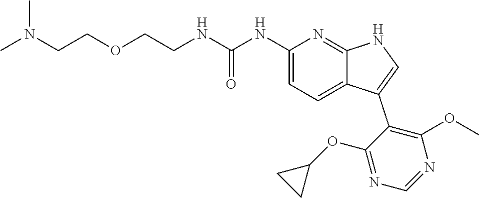 |
| 168 | 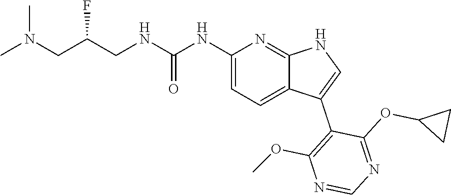 [SMILES: COc1ncnc(OC2CC2)c1-c1c[nH]c2nc(NC(=O)NC[C@@H](F)CN(C)C)ccc12] |
| 168-1 | 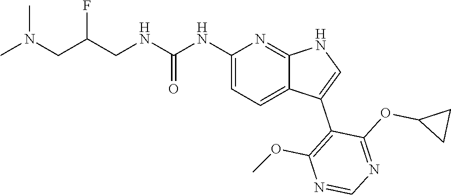 [SMILES: COc1ncnc(OC2CC2)c1-c1c[nH]c2nc(NC(=O)NCC(F)CN(C)C)ccc12] |
| 169 | 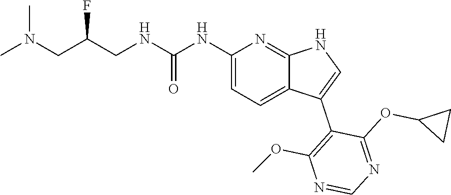 [SMILES: COc1ncnc(OC2CC2)c1-c1c[nH]c2nc(NC(=O)NC[C@H](F)CN(C)C)ccc12] |
| 168-1 | 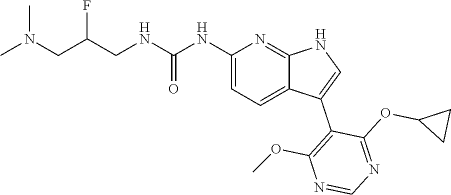 [SMILES: COc1ncnc(OC2CC2)c1-c1c[nH]c2nc(NC(=O)NCC(F)CN(C)C)ccc12] |
| 170 | 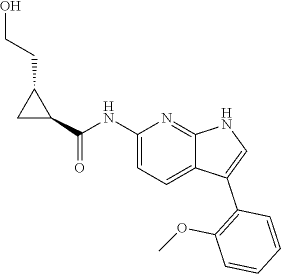 [SMILES: COc1ccccc1-c1c[nH]c2nc(NC(=O)[C@H]3C[C@@H]3CCO)ccc12] |
| 170-1 | 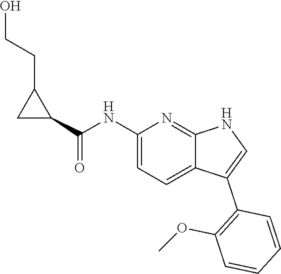 [SMILES: COc1ccccc1-c1c[nH]c2nc(NC(=O)[C@H]3CC3CCO)ccc12] |
| 171 | 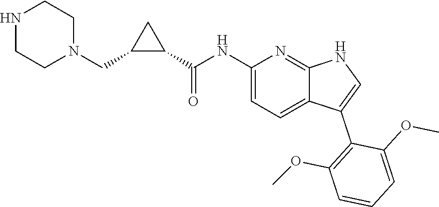 [SMILES: COc1cccc(OC)c1-c1c[nH]c2nc(NC(=O)[C@H]3C[C@H]3CN3CCNCC3)ccc12] |
| 171-1 | 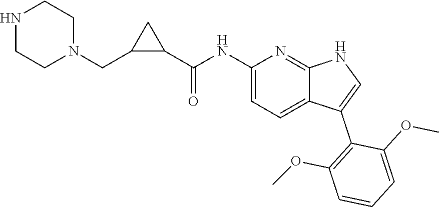 [SMILES: COc1cccc(OC)c1-c1c[nH]c2nc(NC(=O)C3CC3CN3CCNCC3)ccc12] |
| 172 | 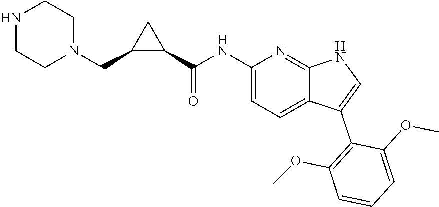 [SMILES: COc1cccc(OC)c1-c1c[nH]c2nc(NC(=O)[C@@H]3C[C@@H]3CN3CCNCC3)ccc12] |
| 171-1 | 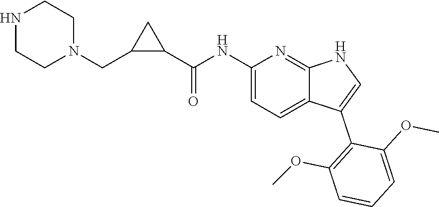 [SMILES: COc1cccc(OC)c1-c1c[nH]c2nc(NC(=O)C3CC3CN3CCNCC3)ccc12] |
| 173 | 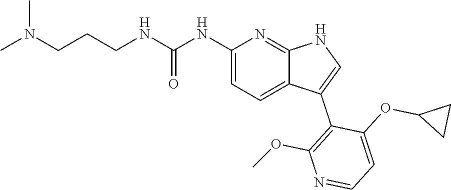 [SMILES: COc1nccc(OC2CC2)c1-c1c[nH]c2nc(NC(=O)NCCCN(C)C)ccc12] |
| 174 | 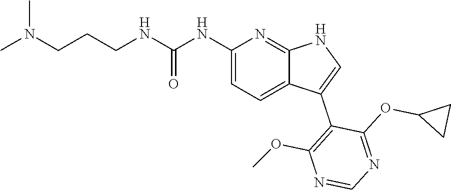 [SMILES: COc1ncnc(OC2CC2)c1-c1c[nH]c2nc(NC(=O)NCCCN(C)C)ccc12] |
| 175 | 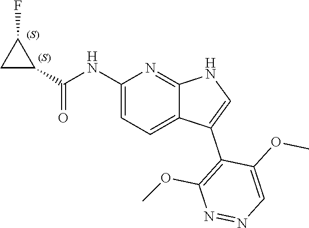 |
| 175-1 | 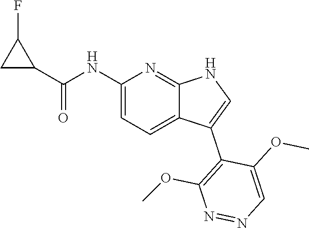 [SMILES: COc1cnnc(OC)c1-c1c[nH]c2nc(NC(=O)C3CC3F)ccc12] |
| 176 | 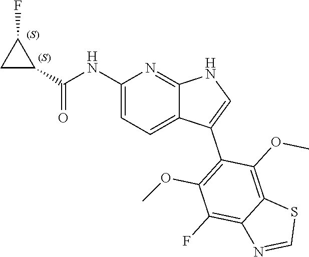 |
| 176-1 | 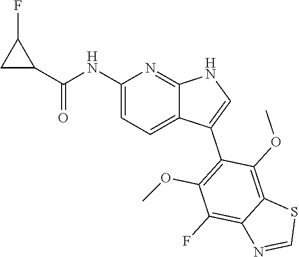 [SMILES: COc1c(-c2c[nH]c3nc(NC(=O)C4CC4F)ccc23)c(OC)c2scnc2c1F] |
| 177 | 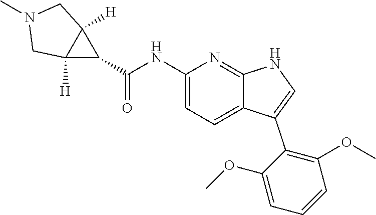 |
| 177-1 | 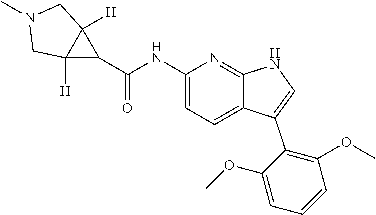 |
| 178 | 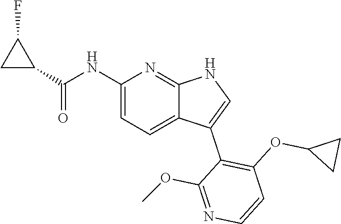 [SMILES: COc1nccc(OC2CC2)c1-c1c[nH]c2nc(NC(=O)[C@@H]3C[C@@H]3F)ccc12] |
| 178-1 | 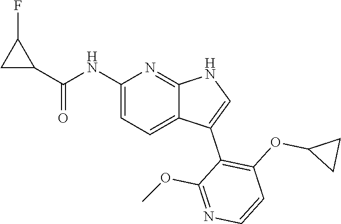 [SMILES: COc1nccc(OC2CC2)c1-c1c[nH]c2nc(NC(=O)C3CC3F)ccc12] |
| 179 | 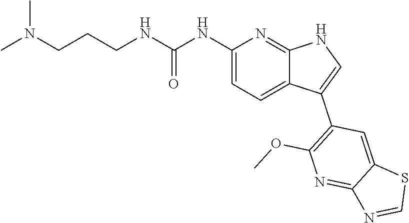 [SMILES: COc1nc2ncsc2cc1-c1c[nH]c2nc(NC(=O)NCCCN(C)C)ccc12] |
| 180 | 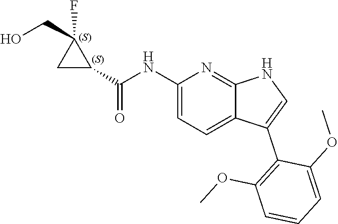 |
| 180-1 | 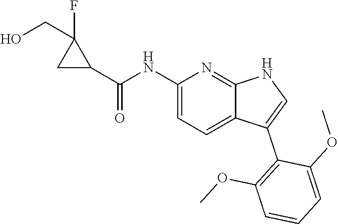 [SMILES: COc1cccc(OC)c1-c1c[nH]c2nc(NC(=O)C3CC3(F)CO)ccc12] |
| 181 | 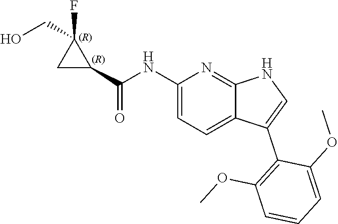 |
| 180-1 | 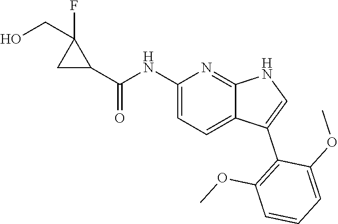 [SMILES: COc1cccc(OC)c1-c1c[nH]c2nc(NC(=O)C3CC3(F)CO)ccc12] |
| 182 | 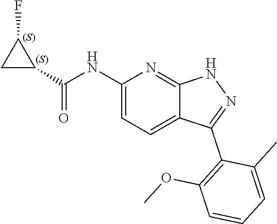 |
| 182-1 | 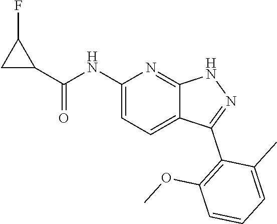 [SMILES: COc1cccc(C)c1-c1n[nH]c2nc(NC(=O)C3CC3F)ccc12] |
| 183 | 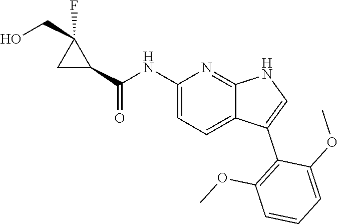 [SMILES: COc1cccc(OC)c1-c1c[nH]c2nc(NC(=O)[C@H]3C[C@@]3(F)CO)ccc12] |
| 180-1 | 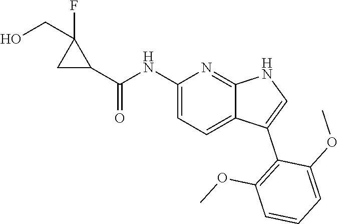 [SMILES: COc1cccc(OC)c1-c1c[nH]c2nc(NC(=O)C3CC3(F)CO)ccc12] |
| 184 | 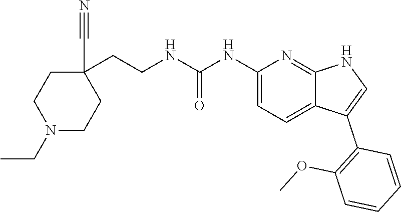 [SMILES: CCN1CCC(C#N)(CCNC(=O)Nc2ccc3c(-c4ccccc4OC)c[nH]c3n2)CC1] |
| 185 | 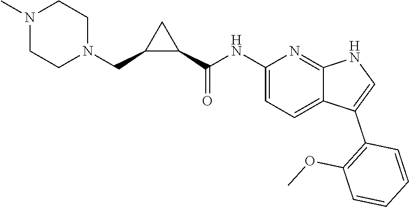 [SMILES: COc1ccccc1-c1c[nH]c2nc(NC(=O)[C@@H]3C[C@@H]3CN3CCN(C)CC3)ccc12] |
| 41-1 | 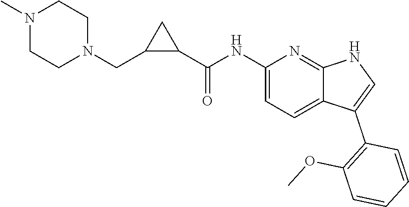 [SMILES: COc1ccccc1-c1c[nH]c2nc(NC(=O)C3CC3CN3CCN(C)CC3)ccc12] |
| 186 | 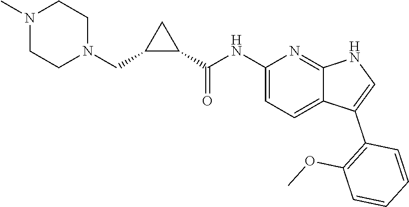 [SMILES: COc1ccccc1-c1c[nH]c2nc(NC(=O)[C@H]3C[C@H]3CN3CCN(C)CC3)ccc12] |
| 41-1 | 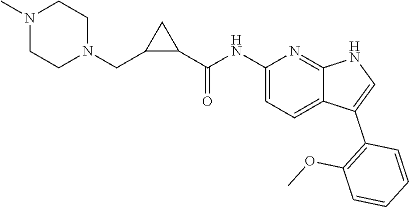 [SMILES: COc1ccccc1-c1c[nH]c2nc(NC(=O)C3CC3CN3CCN(C)CC3)ccc12] |
| 187 | 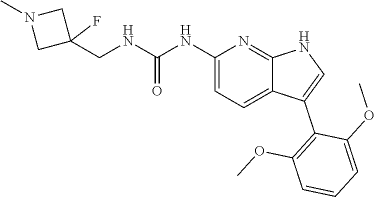 [SMILES: COc1cccc(OC)c1-c1c[nH]c2nc(NC(=O)NCC3(F)CN(C)C3)ccc12] |
| 188 | 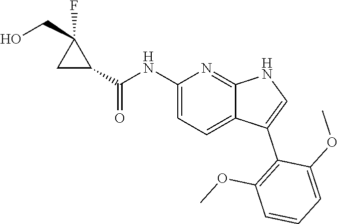 [SMILES: COc1cccc(OC)c1-c1c[nH]c2nc(NC(=O)[C@@H]3C[C@@]3(F)CO)ccc12] |
| 180-1 | 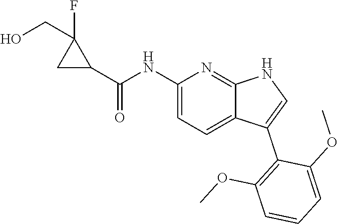 [SMILES: COc1cccc(OC)c1-c1c[nH]c2nc(NC(=O)C3CC3(F)CO)ccc12] |
| 189 | 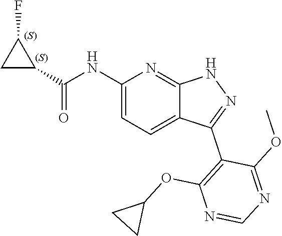 |
| 189-1 | 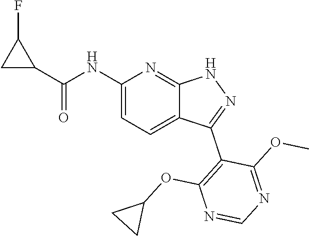 [SMILES: COc1ncnc(OC2CC2)c1-c1n[nH]c2nc(NC(=O)C3CC3F)ccc12] |
| 190 | 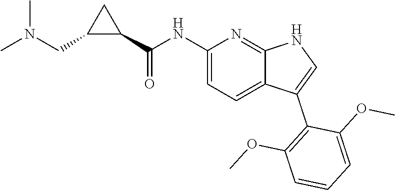 |
| 190-1 | 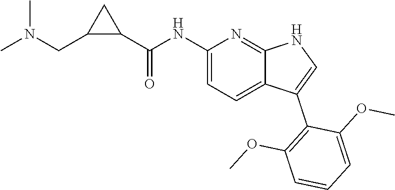 |
| 191 | 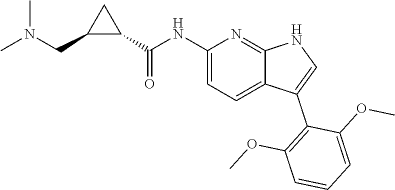 |
| 190-1 | 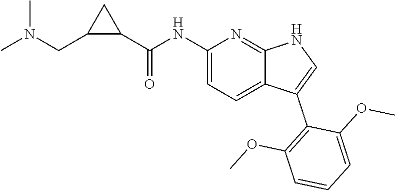 |
| 192 | 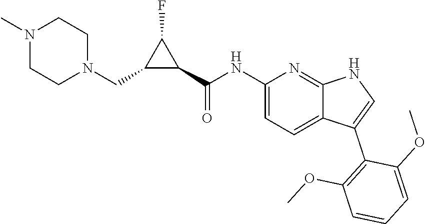 [SMILES: COc1cccc(OC)c1-c1c[nH]c2nc(NC(=O)[C@@H]3[C@@H](F)[C@H]3CN3CCN(C)CC3)ccc12] |
| 192-1 | 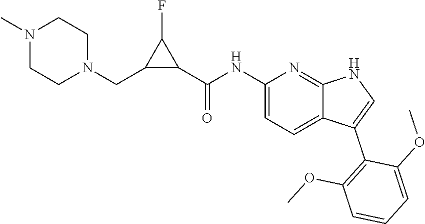 [SMILES: COc1cccc(OC)c1-c1c[nH]c2nc(NC(=O)C3C(F)C3CN3CCN(C)CC3)ccc12] |
| 193 | 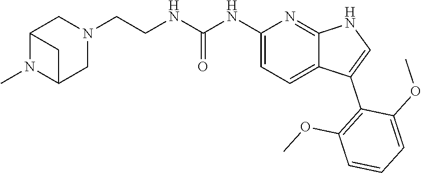 [SMILES: COc1cccc(OC)c1-c1c[nH]c2nc(NC(=O)NCCN3CC4CC(C3)N4C)ccc12] |
| 194 | 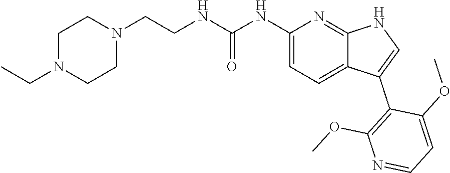 [SMILES: CCN1CCN(CCNC(=O)Nc2ccc3c(-c4c(OC)ccnc4OC)c[nH]c3n2)CC1] |
| 195 | 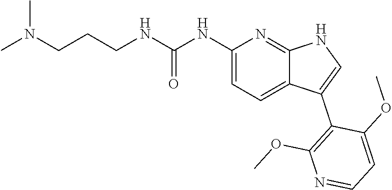 [SMILES: COc1ccnc(OC)c1-c1c[nH]c2nc(NC(=O)NCCCN(C)C)ccc12] |
| 196 | 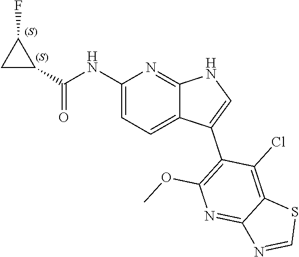 |
| 196-1 | 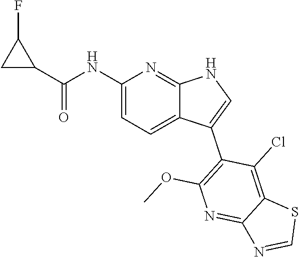 [SMILES: COc1nc2ncsc2c(Cl)c1-c1c[nH]c2nc(NC(=O)C3CC3F)ccc12] |
| 197 | 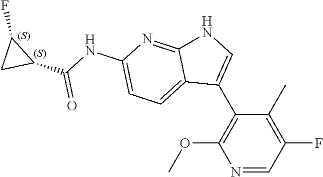 |
| 197-1 | 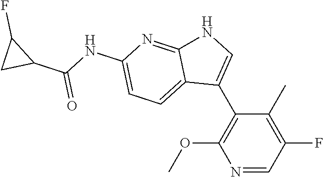 [SMILES: COc1ncc(F)c(C)c1-c1c[nH]c2nc(NC(=O)C3CC3F)ccc12] |
| 198 | 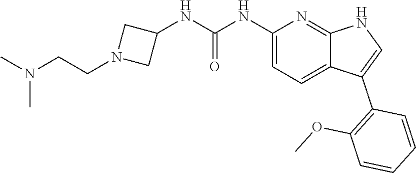 [SMILES: COc1ccccc1-c1c[nH]c2nc(NC(=O)NC3CN(CCN(C)C)C3)ccc12] |
| 199 | 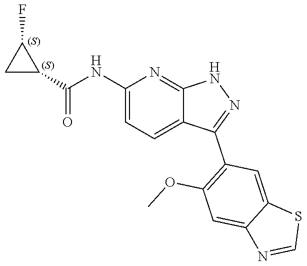 |
| 199-1 | 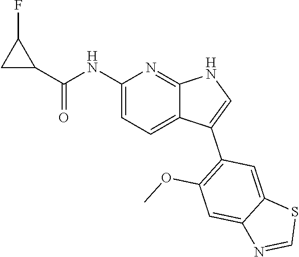 [SMILES: COc1cc2ncsc2cc1-c1c[nH]c2nc(NC(=O)C3CC3F)ccc12] |
| 200 | 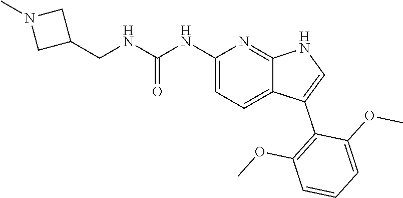 |
| 201 | 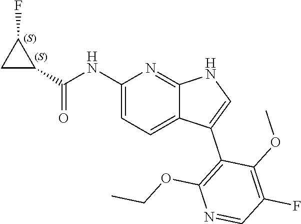 |
| 201-1 | 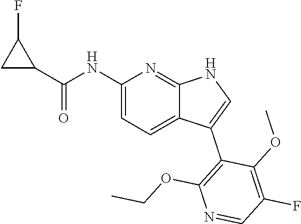 [SMILES: CCOc1ncc(F)c(OC)c1-c1c[nH]c2nc(NC(=O)C3CC3F)ccc12] |
| 202 | 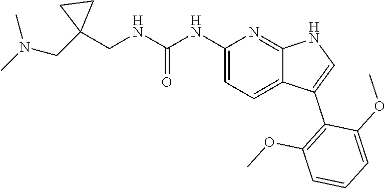 [SMILES: COc1cccc(OC)c1-c1c[nH]c2nc(NC(=O)NCC3(CN(C)C)CC3)ccc12] |
| 203 | 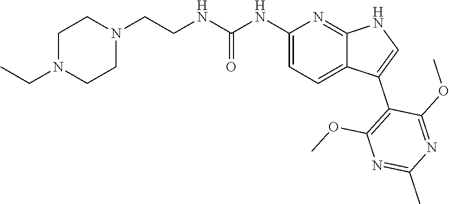 [SMILES: CCN1CCN(CCNC(=O)Nc2ccc3c(-c4c(OC)nc(C)nc4OC)c[nH]c3n2)CC1] |
| 204 | 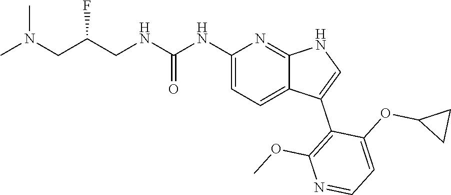 [SMILES: COc1nccc(OC2CC2)c1-c1c[nH]c2nc(NC(=O)NC[C@@H](F)CN(C)C)ccc12] |
| 204-1 | 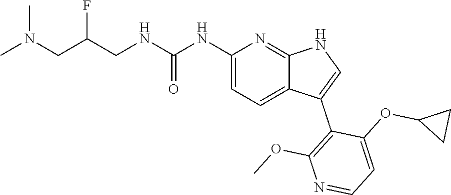 [SMILES: COc1nccc(OC2CC2)c1-c1c[nH]c2nc(NC(=O)NCC(F)CN(C)C)ccc12] |
| 205 | 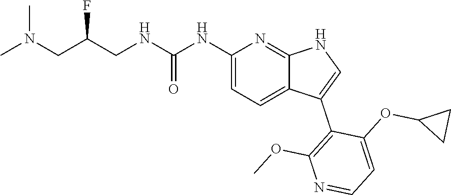 [SMILES: COc1nccc(OC2CC2)c1-c1c[nH]c2nc(NC(=O)NC[C@H](F)CN(C)C)ccc12] |
| 204-1 | 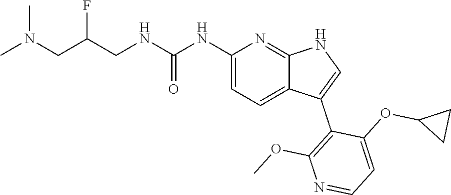 [SMILES: COc1nccc(OC2CC2)c1-c1c[nH]c2nc(NC(=O)NCC(F)CN(C)C)ccc12] |
| 206 | 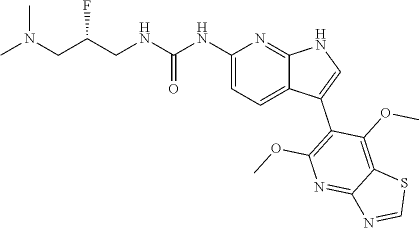 |
| 206-1 | 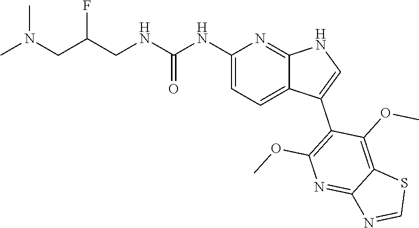 |
| 207 | 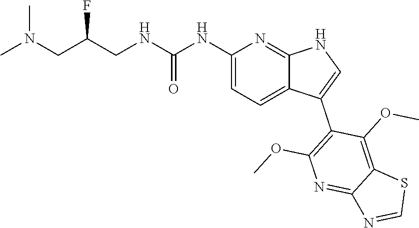 |
| 206-1 | 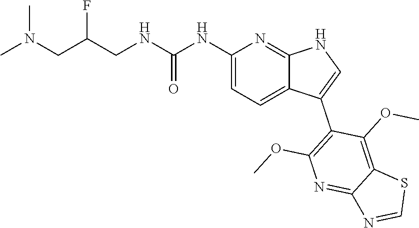 |
| 208 | 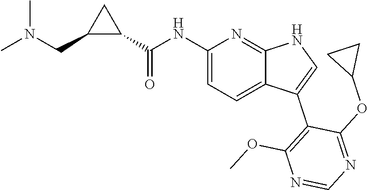 [SMILES: COc1ncnc(OC2CC2)c1-c1c[nH]c2nc(NC(=O)[C@H]3C[C@@H]3CN(C)C)ccc12] |
| 208-1 | 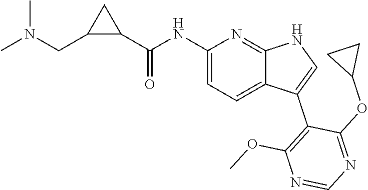 [SMILES: COc1ncnc(OC2CC2)c1-c1c[nH]c2nc(NC(=O)C3CC3CN(C)C)ccc12] |
| 209 | 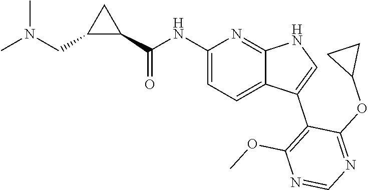 [SMILES: COc1ncnc(OC2CC2)c1-c1c[nH]c2nc(NC(=O)[C@@H]3C[C@H]3CN(C)C)ccc12] |
| 208-1 | 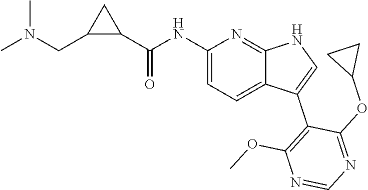 [SMILES: COc1ncnc(OC2CC2)c1-c1c[nH]c2nc(NC(=O)C3CC3CN(C)C)ccc12] |
| 210 | 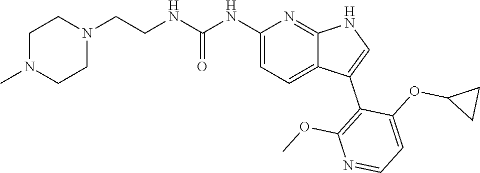 [SMILES: COc1nccc(OC2CC2)c1-c1c[nH]c2nc(NC(=O)NCCN3CCN(C)CC3)ccc12] |
| 211 | 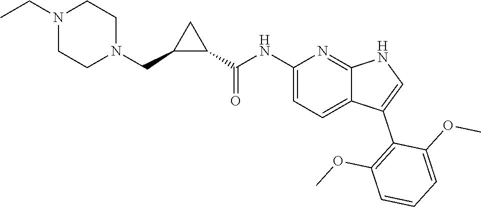 [SMILES: CCN1CCN(C[C@H]2C[C@@H]2C(=O)Nc2ccc3c(-c4c(OC)cccc4OC)c[nH]c3n2)CC1] |
| 211-1 | 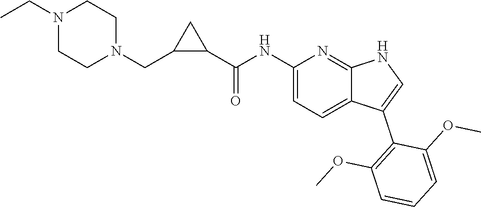 [SMILES: CCN1CCN(CC2CC2C(=O)Nc2ccc3c(-c4c(OC)cccc4OC)c[nH]c3n2)CC1] |
| 212 | 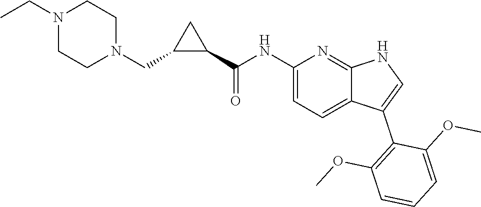 [SMILES: CCN1CCN(C[C@@H]2C[C@H]2C(=O)Nc2ccc3c(-c4c(OC)cccc4OC)c[nH]c3n2)CC1] |
| 211-1 | 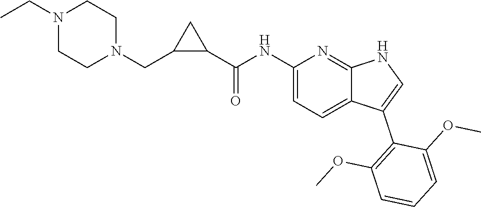 [SMILES: CCN1CCN(CC2CC2C(=O)Nc2ccc3c(-c4c(OC)cccc4OC)c[nH]c3n2)CC1] |
| 213 | 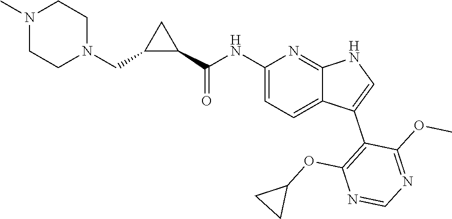 |
| 213-1 | 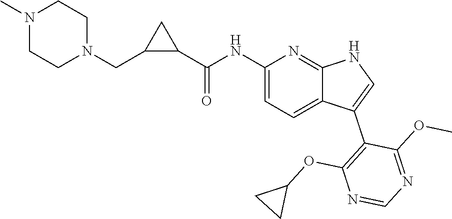 |
| 214 | 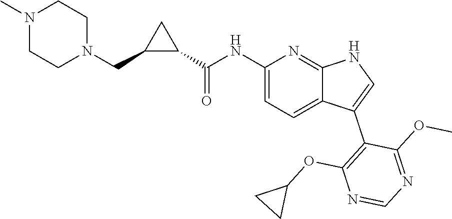 |
| 213-1 | 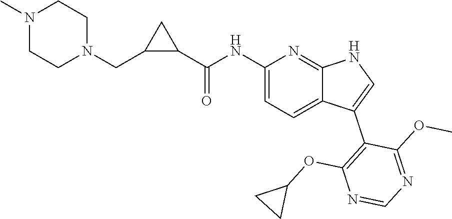 |
| 215 | 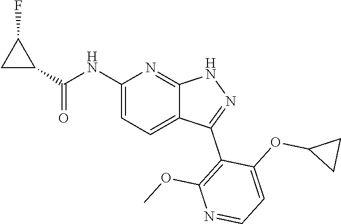 [SMILES: COc1nccc(OC2CC2)c1-c1n[nH]c2nc(NC(=O)[C@@H]3C[C@@H]3F)ccc12] |
| 215-1 | 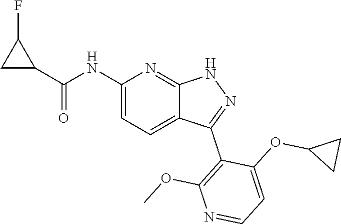 [SMILES: COc1nccc(OC2CC2)c1-c1n[nH]c2nc(NC(=O)C3CC3F)ccc12] |
| 216 | 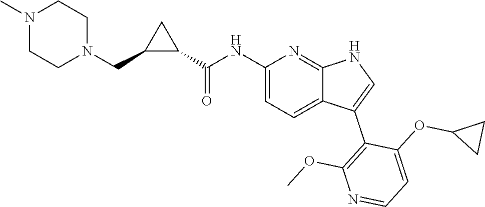 [SMILES: COc1nccc(OC2CC2)c1-c1c[nH]c2nc(NC(=O)[C@H]3C[C@@H]3CN3CCN(C)CC3)ccc12] |
| 216-1 | 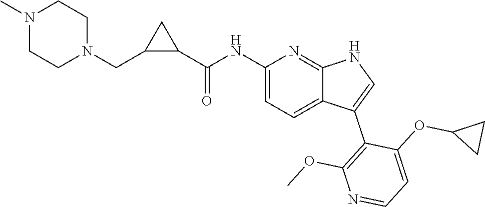 [SMILES: COc1nccc(OC2CC2)c1-c1c[nH]c2nc(NC(=O)C3CC3CN3CCN(C)CC3)ccc12] |
| 217 | 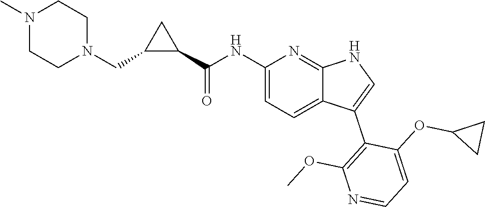 [SMILES: COc1nccc(OC2CC2)c1-c1c[nH]c2nc(NC(=O)[C@@H]3C[C@H]3CN3CCN(C)CC3)ccc12] |
| 216-1 | 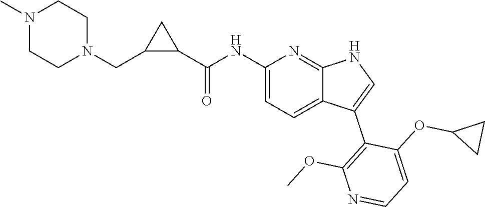 [SMILES: COc1nccc(OC2CC2)c1-c1c[nH]c2nc(NC(=O)C3CC3CN3CCN(C)CC3)ccc12] |
| 218 | 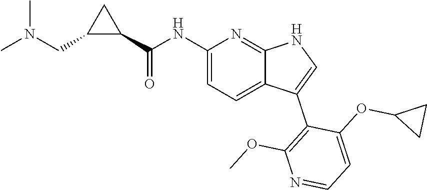 [SMILES: COc1nccc(OC2CC2)c1-c1c[nH]c2nc(NC(=O)[C@@H]3C[C@H]3CN(C)C)ccc12] |
| 218-1 | 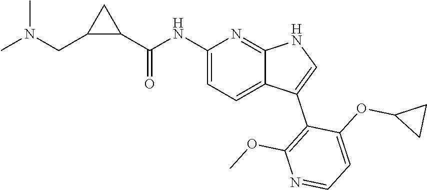 [SMILES: COc1nccc(OC2CC2)c1-c1c[nH]c2nc(NC(=O)C3CC3CN(C)C)ccc12] |
| 219 | 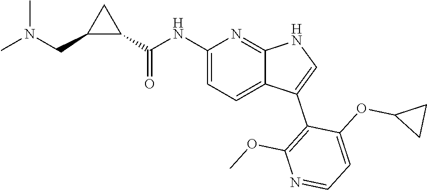 [SMILES: COc1nccc(OC2CC2)c1-c1c[nH]c2nc(NC(=O)[C@H]3C[C@@H]3CN(C)C)ccc12] |
| 218-1 | 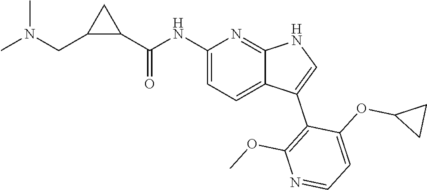 [SMILES: COc1nccc(OC2CC2)c1-c1c[nH]c2nc(NC(=O)C3CC3CN(C)C)ccc12] |
| 220 | 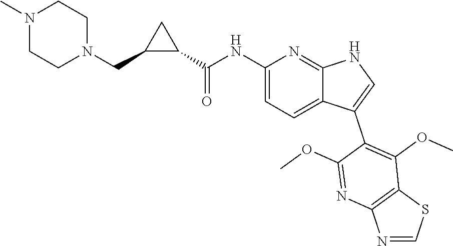 |
| 220-1 | 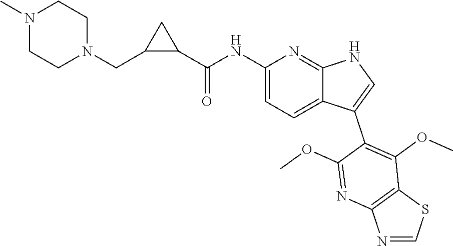 |
| 221 | 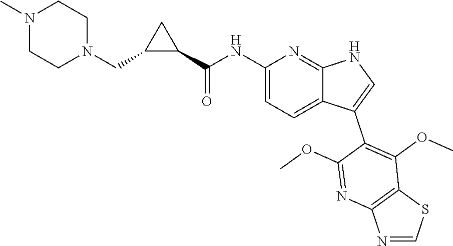 |
| 220-1 | 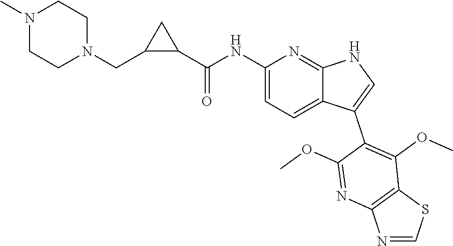 |
| 222 | 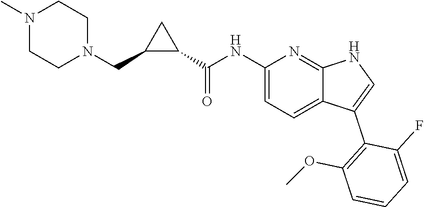 [SMILES: COc1cccc(F)c1-c1c[nH]c2nc(NC(=O)[C@H]3C[C@@H]3CN3CCN(C)CC3)ccc12] |
| 222-1 | 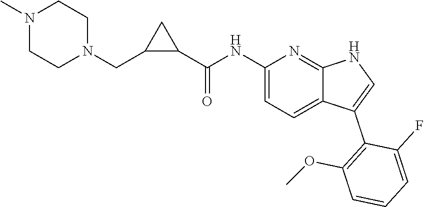 [SMILES: COc1cccc(F)c1-c1c[nH]c2nc(NC(=O)C3CC3CN3CCN(C)CC3)ccc12] |
| 223 | 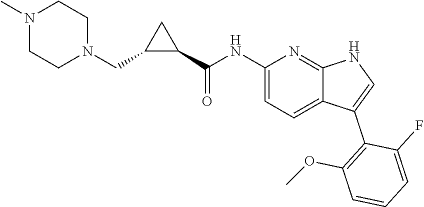 [SMILES: COc1cccc(F)c1-c1c[nH]c2nc(NC(=O)[C@@H]3C[C@H]3CN3CCN(C)CC3)ccc12] |
| 222-1 | 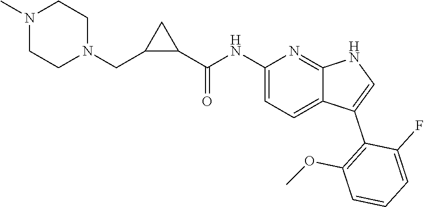 [SMILES: COc1cccc(F)c1-c1c[nH]c2nc(NC(=O)C3CC3CN3CCN(C)CC3)ccc12] |
| 224 | 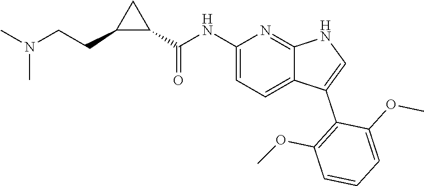 |
| 224-1 | 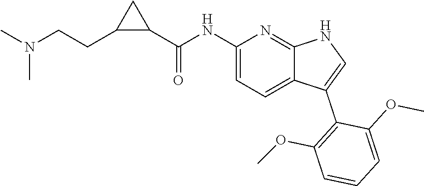 |
| 225 | 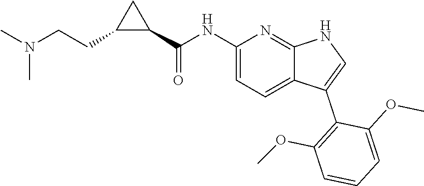 |
| 224-1 | 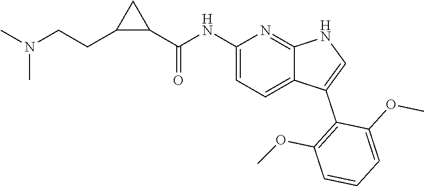 |
| 226 | 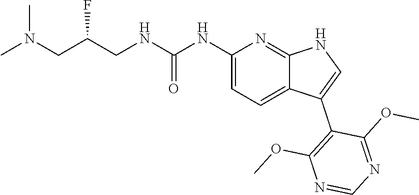 |
| 226-1 | 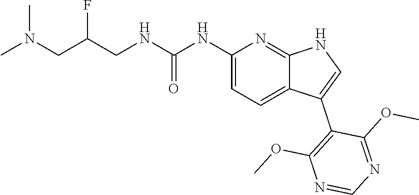 |
| 227 | 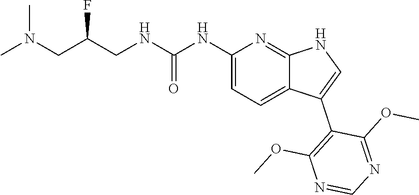 |
| 226-1 | 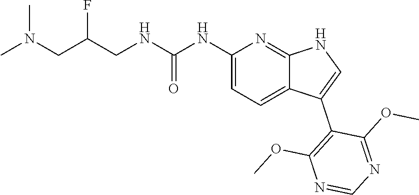 |
| 228 | 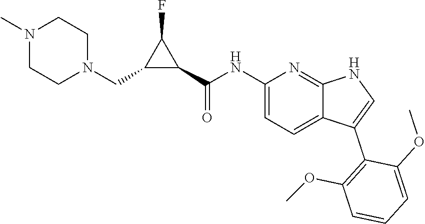 [SMILES: COc1cccc(OC)c1-c1c[nH]c2nc(NC(=O)[C@@H]3[C@H](F)[C@H]3CN3CCN(C)CC3)ccc12] |
| 192-1 | 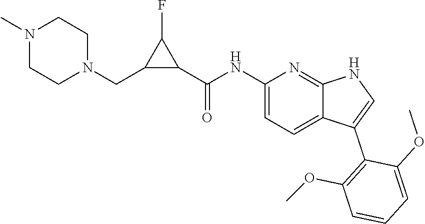 [SMILES: COc1cccc(OC)c1-c1c[nH]c2nc(NC(=O)C3C(F)C3CN3CCN(C)CC3)ccc12] |
| 229 | 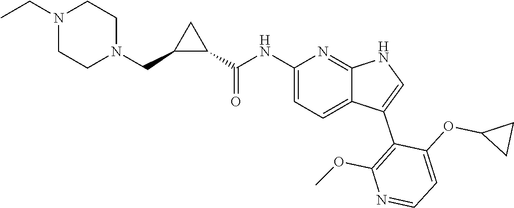 [SMILES: CCN1CCN(C[C@H]2C[C@@H]2C(=O)Nc2ccc3c(-c4c(OC5CC5)ccnc4OC)c[nH]c3n2)CC1] |
| 229-1 | 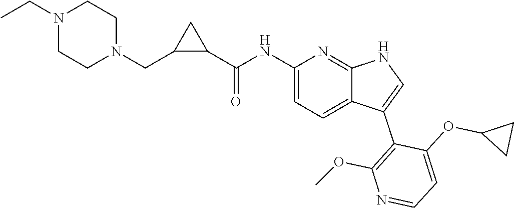 [SMILES: CCN1CCN(CC2CC2C(=O)Nc2ccc3c(-c4c(OC5CC5)ccnc4OC)c[nH]c3n2)CC1] |
| 230 | 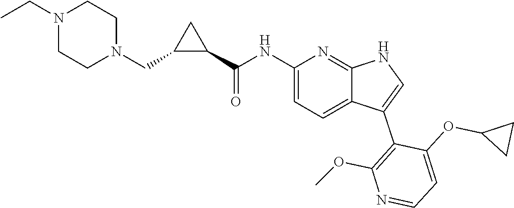 [SMILES: CCN1CCN(C[C@@H]2C[C@H]2C(=O)Nc2ccc3c(-c4c(OC5CC5)ccnc4OC)c[nH]c3n2)CC1] |
| 229-1 | 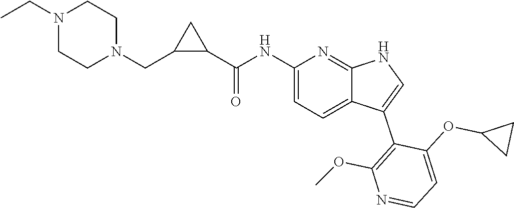 [SMILES: CCN1CCN(CC2CC2C(=O)Nc2ccc3c(-c4c(OC5CC5)ccnc4OC)c[nH]c3n2)CC1] |
| 231 | 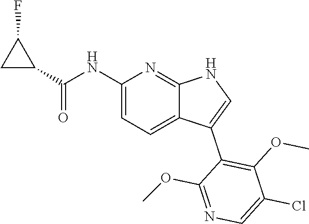 [SMILES: COc1ncc(Cl)c(OC)c1-c1c[nH]c2nc(NC(=O)[C@@H]3C[C@@H]3F)ccc12] |
| 231-1 | 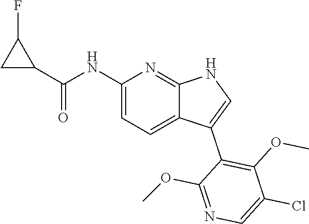 [SMILES: COc1ncc(Cl)c(OC)c1-c1c[nH]c2nc(NC(=O)C3CC3F)ccc12] |
| 232 | 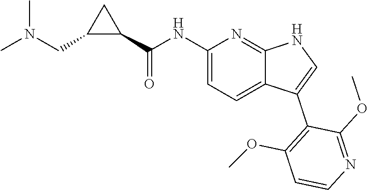 [SMILES: COc1ccnc(OC)c1-c1c[nH]c2nc(NC(=O)[C@@H]3C[C@H]3CN(C)C)ccc12] |
| 232-1 | 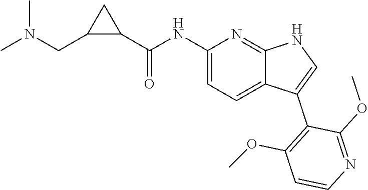 [SMILES: COc1ccnc(OC)c1-c1c[nH]c2nc(NC(=O)C3CC3CN(C)C)ccc12] |
| 233 | 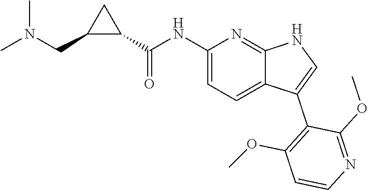 [SMILES: COc1ccnc(OC)c1-c1c[nH]c2nc(NC(=O)[C@H]3C[C@@H]3CN(C)C)ccc12] |
| 232-1 | 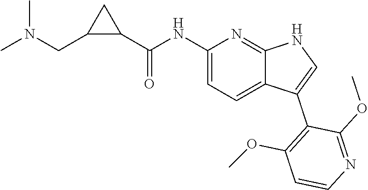 [SMILES: COc1ccnc(OC)c1-c1c[nH]c2nc(NC(=O)C3CC3CN(C)C)ccc12] |
| 234 | 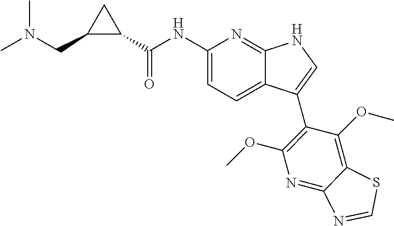 |
| 234-1 | 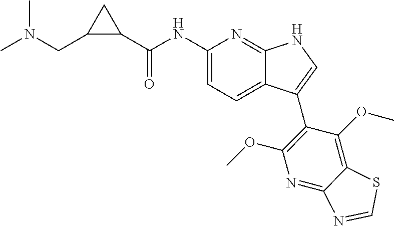 |
| 235 | 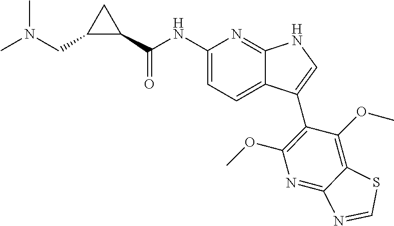 |
| 234-1 | 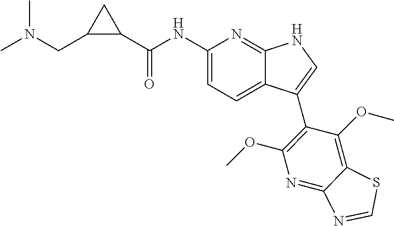 |
| 236 | 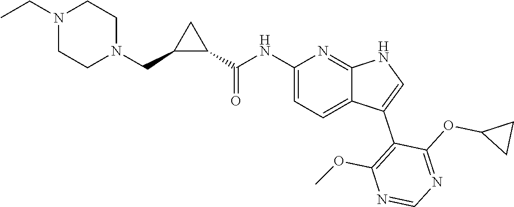 [SMILES: CCN1CCN(C[C@H]2C[C@@H]2C(=O)Nc2ccc3c(-c4c(OC)ncnc4OC4CC4)c[nH]c3n2)CC1] |
| 236-1 | 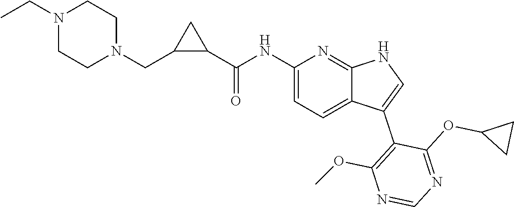 [SMILES: CCN1CCN(CC2CC2C(=O)Nc2ccc3c(-c4c(OC)ncnc4OC4CC4)c[nH]c3n2)CC1] |
| 237 | 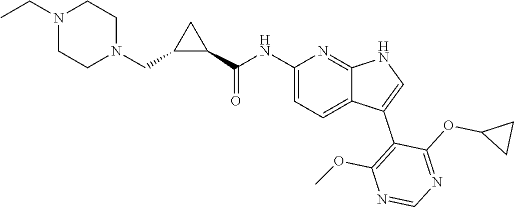 [SMILES: CCN1CCN(C[C@@H]2C[C@H]2C(=O)Nc2ccc3c(-c4c(OC)ncnc4OC4CC4)c[nH]c3n2)CC1] |
| 236-1 | 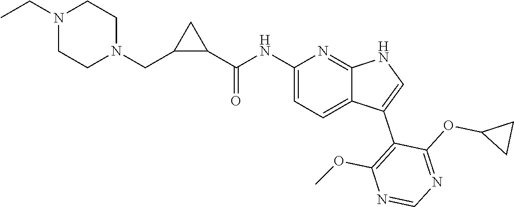 [SMILES: CCN1CCN(CC2CC2C(=O)Nc2ccc3c(-c4c(OC)ncnc4OC4CC4)c[nH]c3n2)CC1] |
| 238 | 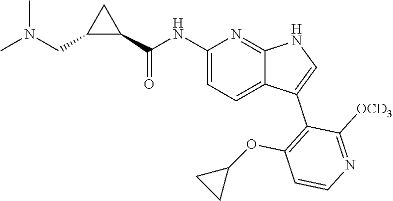 |
| 238-1 | 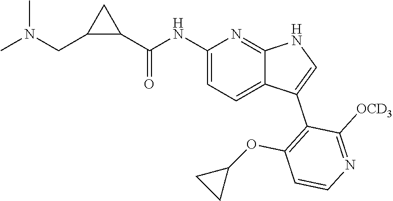 |
| 239 | 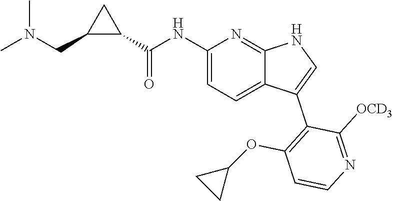 |
| 238-1 | 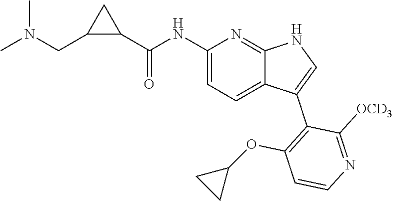 |
| 240 | 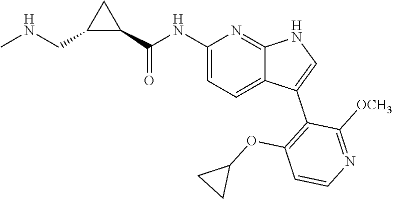 [SMILES: CNC[C@@H]1C[C@H]1C(=O)Nc1ccc2c(-c3c(OC4CC4)ccnc3OC)c[nH]c2n1] |
| 240-1 | 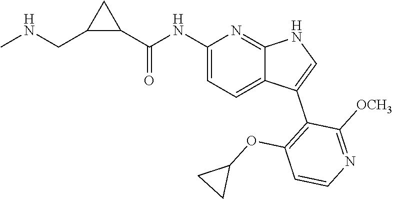 [SMILES: CNCC1CC1C(=O)Nc1ccc2c(-c3c(OC4CC4)ccnc3OC)c[nH]c2n1] |
| 241 | 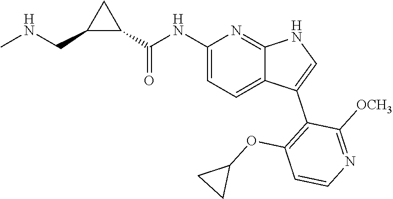 [SMILES: CNC[C@H]1C[C@@H]1C(=O)Nc1ccc2c(-c3c(OC4CC4)ccnc3OC)c[nH]c2n1] |
| 240-1 | 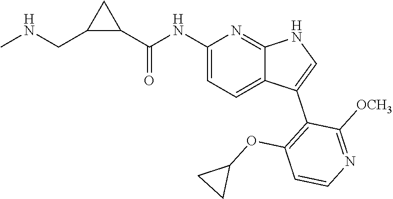 [SMILES: CNCC1CC1C(=O)Nc1ccc2c(-c3c(OC4CC4)ccnc3OC)c[nH]c2n1] |
| 242 | 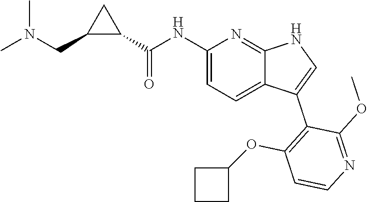 [SMILES: COc1nccc(OC2CCC2)c1-c1c[nH]c2nc(NC(=O)[C@H]3C[C@@H]3CN(C)C)ccc12] |
| 242-1 | 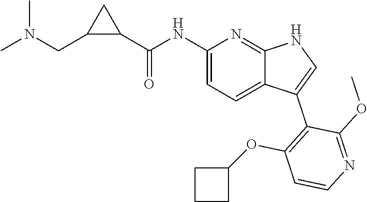 [SMILES: COc1nccc(OC2CCC2)c1-c1c[nH]c2nc(NC(=O)C3CC3CN(C)C)ccc12] |
| 243 | 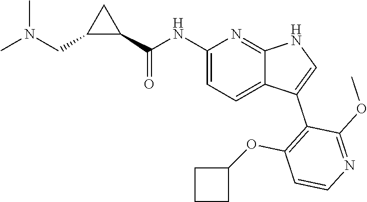 [SMILES: COc1nccc(OC2CCC2)c1-c1c[nH]c2nc(NC(=O)[C@@H]3C[C@H]3CN(C)C)ccc12] |
| 242-1 | 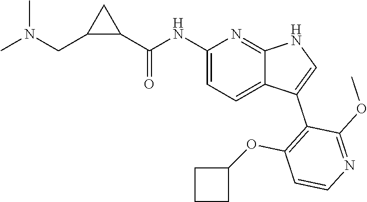 [SMILES: COc1nccc(OC2CCC2)c1-c1c[nH]c2nc(NC(=O)C3CC3CN(C)C)ccc12] |
| 244 | 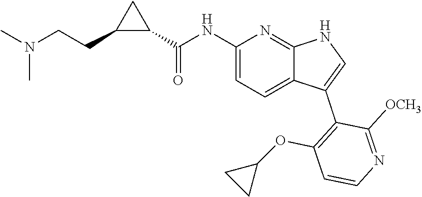 [SMILES: COc1nccc(OC2CC2)c1-c1c[nH]c2nc(NC(=O)[C@H]3C[C@@H]3CCN(C)C)ccc12] |
| 244-1 | 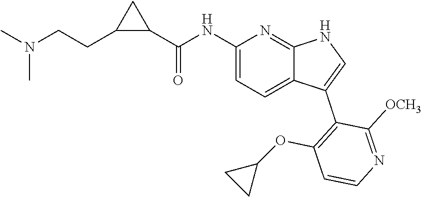 [SMILES: COc1nccc(OC2CC2)c1-c1c[nH]c2nc(NC(=O)C3CC3CCN(C)C)ccc12] |
| 245 | 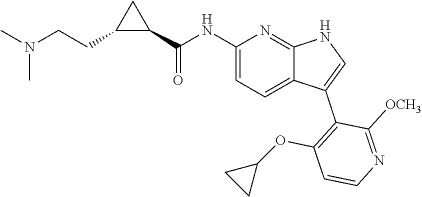 [SMILES: COc1nccc(OC2CC2)c1-c1c[nH]c2nc(NC(=O)[C@@H]3C[C@H]3CCN(C)C)ccc12] |
| 244-1 | 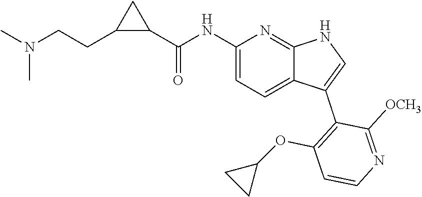 [SMILES: COc1nccc(OC2CC2)c1-c1c[nH]c2nc(NC(=O)C3CC3CCN(C)C)ccc12] |
| 246 | 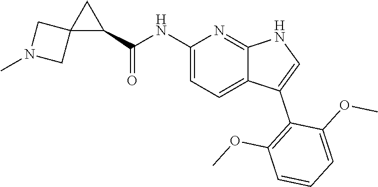 |
| 246-1 | 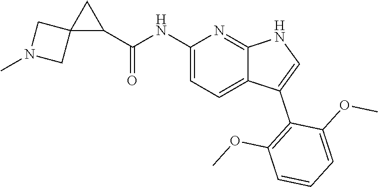 |
| 247 | 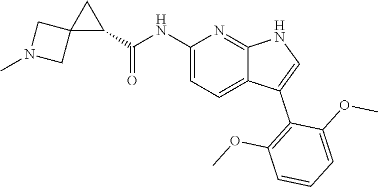 |
| 246-1 | 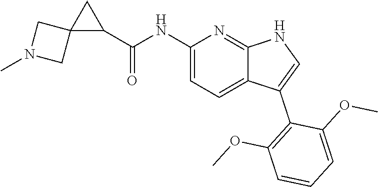 |
| 248 | 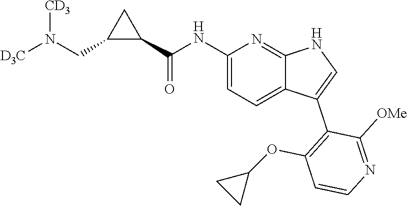 [SMILES: [2H]C([2H])([2H])N(C[C@@H]1C[C@H]1C(=O)Nc1ccc2c(-c3c(OC4CC4)ccnc3OC)c[nH]c2n1)C([2H])([2H])[2H]] |
| 248-1 | 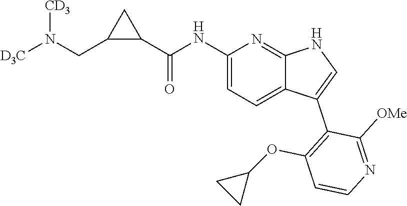 [SMILES: [2H]C([2H])([2H])N(CC1CC1C(=O)Nc1ccc2c(-c3c(OC4CC4)ccnc3OC)c[nH]c2n1)C([2H])([2H])[2H]] |
| 249 | 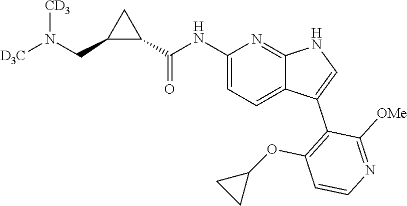 [SMILES: [2H]C([2H])([2H])N(C[C@H]1C[C@@H]1C(=O)Nc1ccc2c(-c3c(OC4CC4)ccnc3OC)c[nH]c2n1)C([2H])([2H])[2H]] |
| 248-1 | 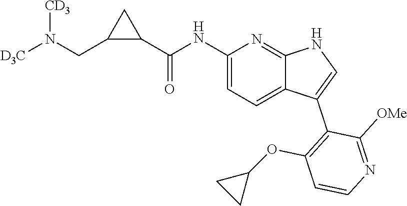 [SMILES: [2H]C([2H])([2H])N(CC1CC1C(=O)Nc1ccc2c(-c3c(OC4CC4)ccnc3OC)c[nH]c2n1)C([2H])([2H])[2H]] |
| 250 | 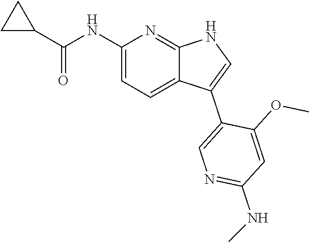 [SMILES: CNc1cc(OC)c(-c2c[nH]c3nc(NC(=O)C4CC4)ccc23)cn1] |
| 251 | 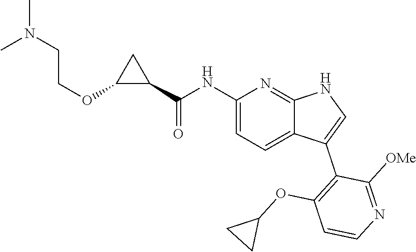 [SMILES: COc1nccc(OC2CC2)c1-c1c[nH]c2nc(NC(=O)[C@@H]3C[C@H]3OCCN(C)C)ccc12] |
| 251-1 | 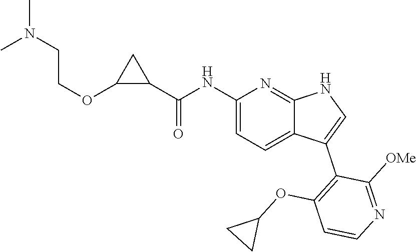 [SMILES: COc1nccc(OC2CC2)c1-c1c[nH]c2nc(NC(=O)C3CC3OCCN(C)C)ccc12] |
| 252 | 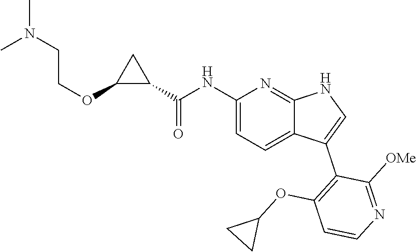 [SMILES: COc1nccc(OC2CC2)c1-c1c[nH]c2nc(NC(=O)[C@H]3C[C@@H]3OCCN(C)C)ccc12] |
| 251-1 | 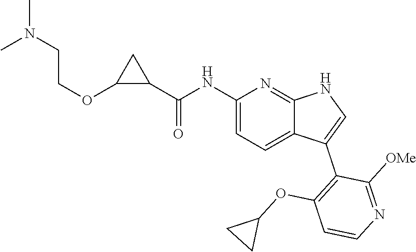 [SMILES: COc1nccc(OC2CC2)c1-c1c[nH]c2nc(NC(=O)C3CC3OCCN(C)C)ccc12] |
| 253 | 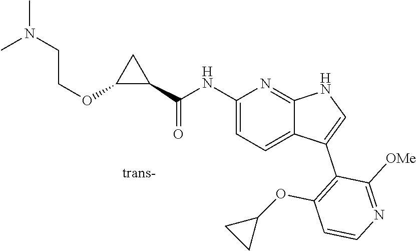 [SMILES: COc1nccc(OC2CC2)c1-c1c[nH]c2nc(NC(=O)[C@@H]3C[C@H]3OCCN(C)C)ccc12] |
| 251-1 | 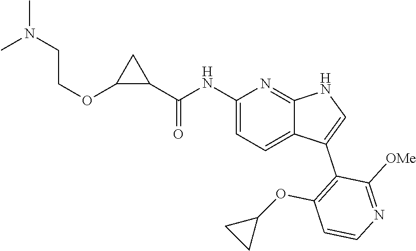 [SMILES: COc1nccc(OC2CC2)c1-c1c[nH]c2nc(NC(=O)C3CC3OCCN(C)C)ccc12] |
| 254 | 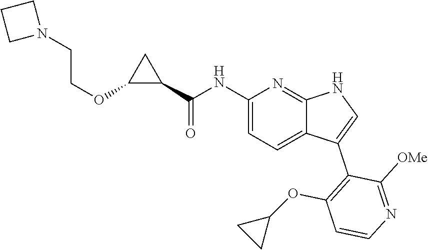 [SMILES: COc1nccc(OC2CC2)c1-c1c[nH]c2nc(NC(=O)[C@@H]3C[C@H]3OCCN3CCC3)ccc12] |
| 254-1 | 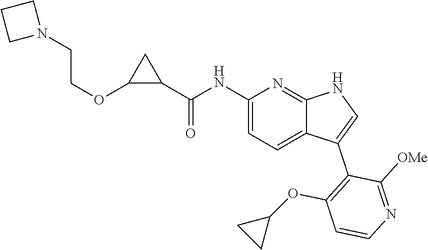 [SMILES: COc1nccc(OC2CC2)c1-c1c[nH]c2nc(NC(=O)C3CC3OCCN3CCC3)ccc12] |
| 255 | 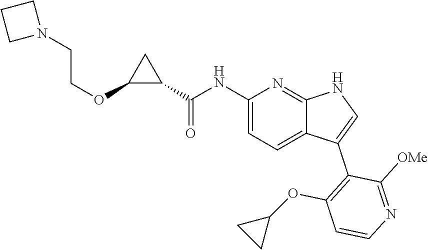 [SMILES: COc1nccc(OC2CC2)c1-c1c[nH]c2nc(NC(=O)[C@H]3C[C@@H]3OCCN3CCC3)ccc12] |
| 254-1 | 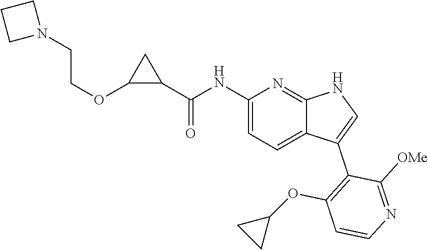 [SMILES: COc1nccc(OC2CC2)c1-c1c[nH]c2nc(NC(=O)C3CC3OCCN3CCC3)ccc12] |
| 256 | 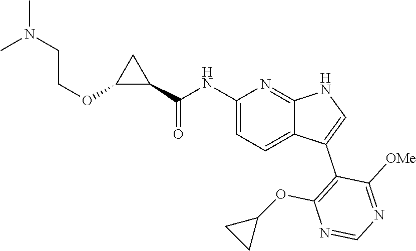 [SMILES: COc1ncnc(OC2CC2)c1-c1c[nH]c2nc(NC(=O)[C@@H]3C[C@H]3OCCN(C)C)ccc12] |
| 256-1 | 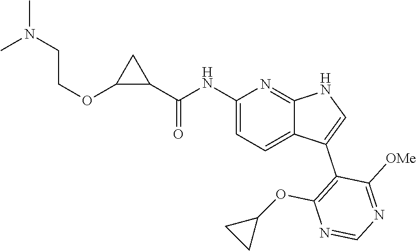 [SMILES: COc1ncnc(OC2CC2)c1-c1c[nH]c2nc(NC(=O)C3CC3OCCN(C)C)ccc12] |
| 257 | 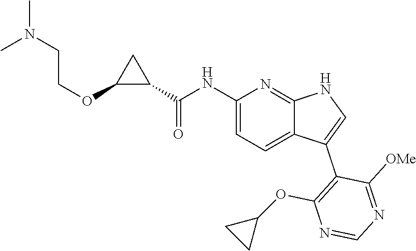 [SMILES: COc1ncnc(OC2CC2)c1-c1c[nH]c2nc(NC(=O)[C@H]3C[C@@H]3OCCN(C)C)ccc12] |
| 256-1 | 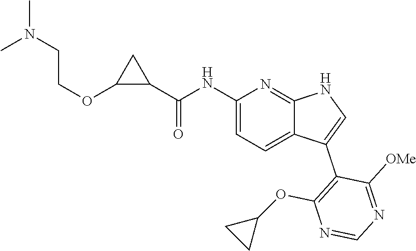 [SMILES: COc1ncnc(OC2CC2)c1-c1c[nH]c2nc(NC(=O)C3CC3OCCN(C)C)ccc12] |
| 258 | 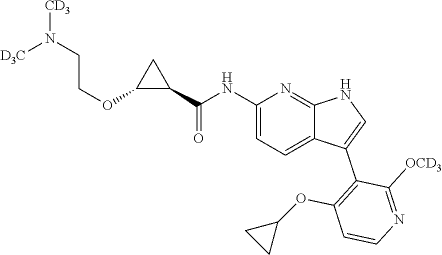 |
| 258-1 | 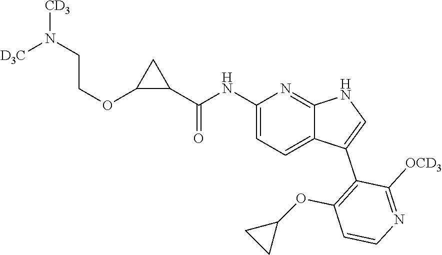 |
| 259 | 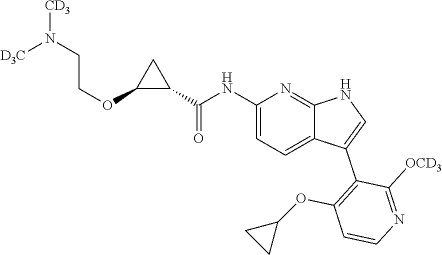 |
| 258-1 | 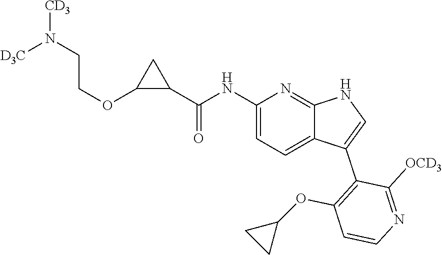 |

Although certain compounds described in Table 1 are presented as specific stereoisomers and/or in a non-stereochemical form, it is understood that any or all non-stereochemical forms and any or all stereochemical forms, including any enantiomeric or diastereomeric forms, and any tautomers or other forms of any of the compounds of Table 1 are herein described. In some embodiments, the compound described herein is selected from Compound Nos. 1-259.

This disclosure also includes all salts, such as pharmaceutically acceptable salts, of compounds referred to herein. This disclosure also includes any or all of the stereochemical forms, including any enantiomeric or diastereomeric forms, and any tautomers or other forms, such as N-oxides, solvates, hydrates, or isotopomers, of the compounds described. The present disclosure also includes co-crystals of the compounds described herein. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also embraced by the invention. All forms of the compounds are also embraced by the invention, such as crystalline or non-crystalline forms of the compounds. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced.

In the descriptions herein, it is understood that every description, variation, embodiment, or aspect of a moiety can be combined with every description, variation, embodiment, or aspect of other moieties the same as if each and every combination of descriptions is specifically and individually listed. For example, every description, variation, embodiment, or aspect provided herein with respect to R⁰ of formula (I) may be combined with every description, variation, embodiment, or aspect of X, m, R¹, R², R³, R⁴, R^{4′}, R⁵, R^{5′}, R⁶, R⁷, and/or R¹, the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, all descriptions, variations, embodiments, or aspects of formula (I), where applicable, apply equally to any of formulae (I-A), (I-A-i), (I-B), (I-B-i) and (I-B-ii) detailed herein, and are equally described, the same as if each and every description, variation, embodiment or aspect were separately and individually listed for all formulae.

### III. General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

The intermediates described in the following preparations may contain a number of nitrogen, hydroxy, and acid protecting groups such as esters. The variable protecting group may be the same or different in each occurrence depending on the particular reaction conditions and the particular transformations to be performed. The protection and deprotection conditions are well known to the skilled artisan and are described in the literature. See e.g., Greene and Wuts, Protective Groups in Organic Synthesis, (T. Greene and P. Wuts, eds., 2d ed. 1991).

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See e.g., J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The compounds of the present invention, or a pharmaceutically acceptable salt, solvate, hydrate, or co-crystal thereof, or a mixture of any of the foregoing, may be prepared by a variety of procedures known in the art, some of which are illustrated in the Examples below. The specific synthetic steps for each of the routes described may be combined in different ways, to prepare compounds of the present disclosure, or salts thereof. The products of each step can be recovered by conventional methods well known in the art, including extraction, evaporation, precipitation, chromatography, filtration, trituration, and crystallization. The reagents and starting materials are readily available to one of ordinary skill in the art. Others may be made by standard techniques of organic and heterocyclic chemistry which are analogous to the syntheses of known structurally-similar compounds and the procedures described in the Examples which follow including any novel procedures.

Compounds of formula (I) can be prepared according to Scheme A, Scheme B, Scheme C, Scheme D, Scheme E, or Scheme F, wherein X, m, R¹, R², R³, R⁴, R^{4′}, R⁵, R^{5′}, R⁶, R⁷, and/or R⁸ are as defined for formula (I) or any applicable variation thereof as detailed herein.

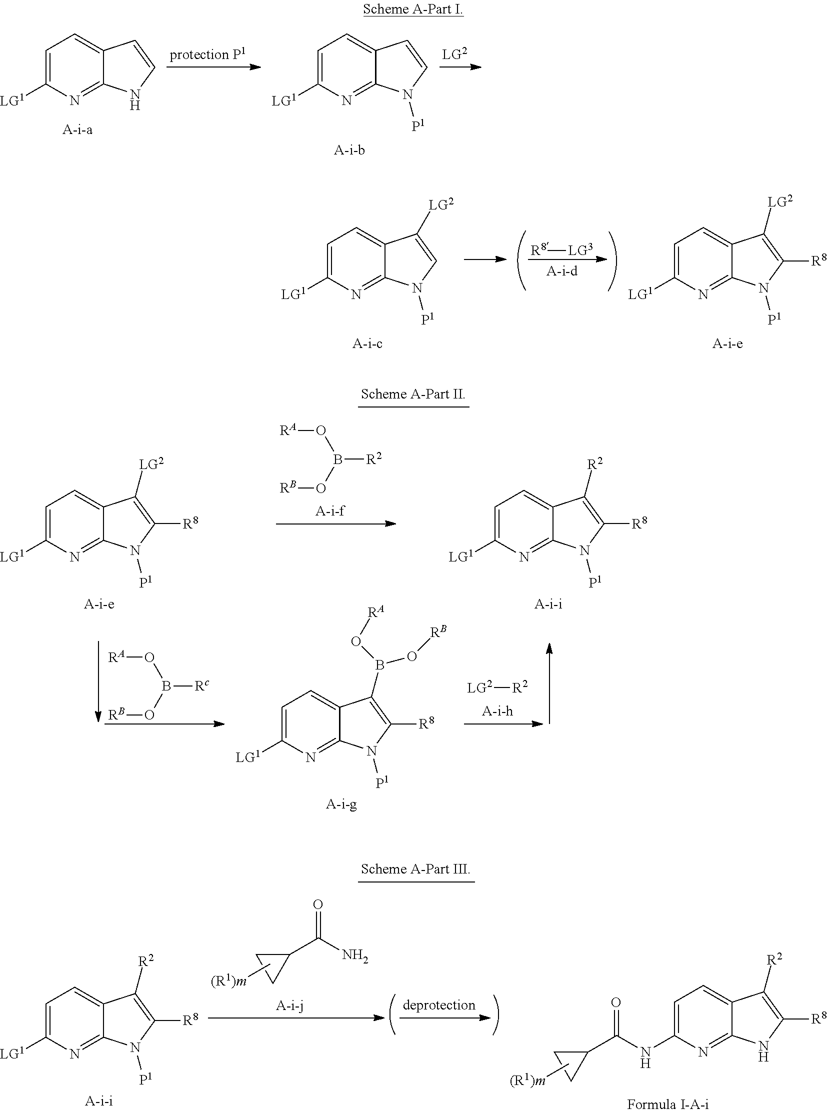

Compounds of formula (I-A-i) may be prepared according to the general synthetic scheme shown in Scheme A, Parts I-III. In Scheme A, Part I, 7-azaindole compounds of general formula A-i-a, wherein LG¹ is a leaving group (such as chloro or bromo), are protected at the secondary nitrogen with a protecting group P¹ (e.g., with a Boc- or SEM-group) to give the compounds of general formula A-i-b. A suitable leaving group LG² (such as iodo) is added to the protected compounds of general formula A-i-b in preparation for the installation of the R² moiety. The resulting compounds of general formula A-i-c are further subjected to reactions to introduce substituent R^{8′} onto the 7-azaindole core as required to produce the intermediate compounds of general formula A-i-e. As shown in Scheme A, R^{8′} has the same definition as R⁸ as described herein, except that R^{8′} does not include hydrogen.

In Part II of Scheme A, the R² aryl or heteroaryl moiety is added to the compounds of general formula A-i-e, at the position occupied by LG², to yield the further intermediate compounds of general formula A-i-i. The installation of the R² moiety may be achieved, for example, by two routes as shown above. In the first route, the compounds of general formula A-i-e are reacted with a suitable boronic acid derivative comprising the desired R² group A-i-f, wherein R^{A} and R^{B} are independently selected from the group consisting of halogen, OH, and O—(C₁-C₆ alkyl), or R^{A} and R^{B} are taken together with the boron atom to which they are attached to form a 5-10 membered heterocycle, to give the intermediate compounds of formula A-i-i. In the second route, the compounds of formula A-i-e are directly reacted with boronic acid or a derivative thereof, wherein R^{c} is a suitable leaving group (such as O—C₁-C₃ alkyl, or another boronic acid or derivative thereof, i.e., in a diboron compound), to give the 7-azaindolyl-boronate compounds of formula A-i-g. The resulting boronate compounds are further reacted with an R²-containing substrate (A-i-h) to give compounds of formula A-i-i.

In Part III of Scheme A, the compounds of formula A-i-i are reacted with suitable cyclopropanecarboxamides of formula A-i-j to provide compounds of formula I-A-i.

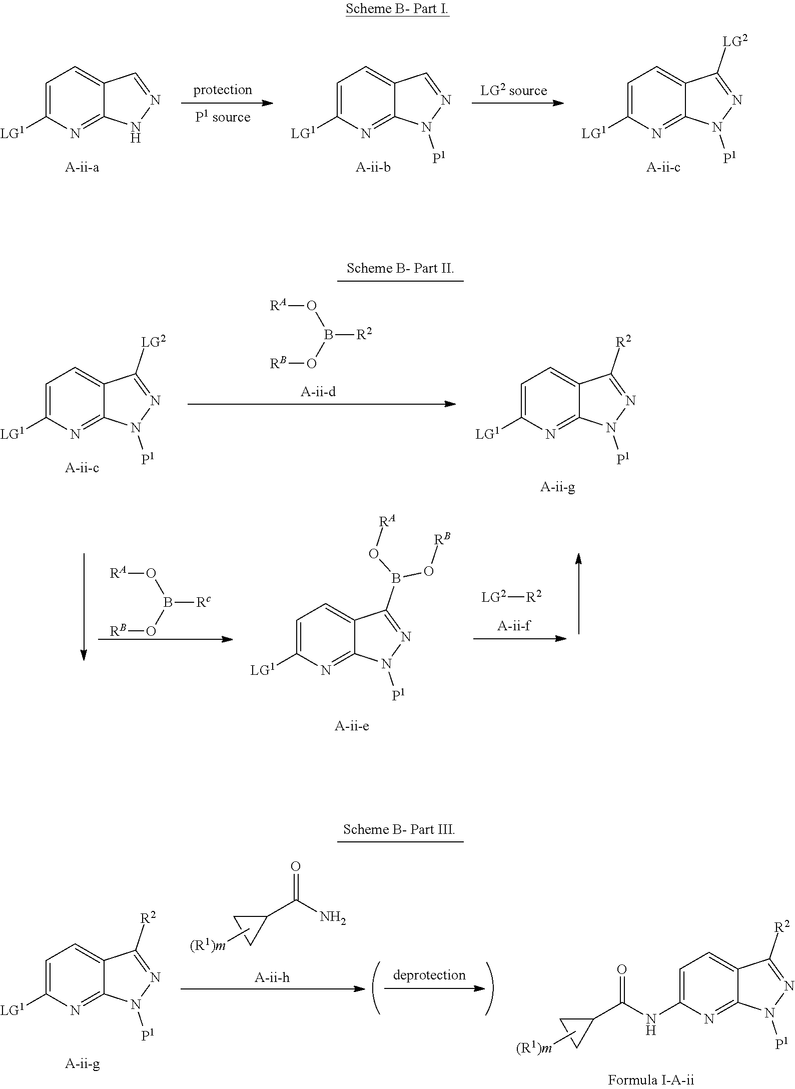

Similar to Scheme A, Scheme B describes the preparation of compounds of formula (I-A-ii) having a 7-azaindazolyl core. Compounds of formula (I-A-ii) may be prepared according to the general synthetic scheme shown in Scheme B, Parts I-III. In Scheme B, Part I, 7-azaindazole compounds of general formula A-ii-a, wherein LG¹ is a leaving group (such as chloro or bromo), are protected at the secondary nitrogen with a protecting group P¹ (e.g., with a Boc- or SEM-group) to give the compounds of general formula A-ii-b. A suitable leaving group LG² (such as iodo) is added to the protected compounds of general formula A-ii-b in preparation for the installation of the R² moiety.

In Part II of Scheme B, the R² aryl or heteroaryl moiety is added to the compounds of general formula A-ii-c, at the position occupied by LG², to yield the further intermediate compounds of general formula A-ii-g. The installation of the R² moiety may be achieved, for example, by two routes as shown above. In the first route, the compounds of general formula A-ii-c are reacted with a suitable boronic acid derivative comprising the desired R² group A-ii-d, wherein R^{A} and R^{B} are independently selected from the group consisting of halogen, OH, and O—(C₁-C₆ alkyl), or R^{A} and R^{B} are taken together with the boron atom to which they are attached to form a 5-10 membered heterocycle, to give the intermediate compounds of formula A-ii-g. In the second route, the compounds of formula A-ii-c are directly reacted with boronic acid or a derivative thereof, wherein R^{c} is a suitable leaving group (such as O—C₁-C₃ alkyl, or another boronic acid or derivative thereof, i.e., in a diboron compound), to give the 7-azaindazolyl-boronate compounds of formula A-ii-e. The resulting boronate compounds are further reacted with an R²-containing substrate (A-ii-f) to give compounds of formula A-ii-g.

In Part III of Scheme B, the compounds of formula A-ii-g are reacted with suitable cyclopropanecarboxamides of formula A-ii-h to provide compounds of formula I-A-ii.

Compounds of formula (I-B) may also be prepared according to the general synthetic schemes shown in Scheme C and Scheme D. In Scheme C, the 7-azaindolyl compounds of formula A-i-i obtained from Scheme A (Parts I and II) are reacted with suitable urea compounds of formula B-i-j to provide compounds of formula I-B-i. In Scheme D, the 7-azaindazolyl compounds of formula A-ii-g obtained from Scheme B (Parts I and II) are reacted with suitable urea compounds of formula B-ii-j to provide compounds of formula I-B-ii.

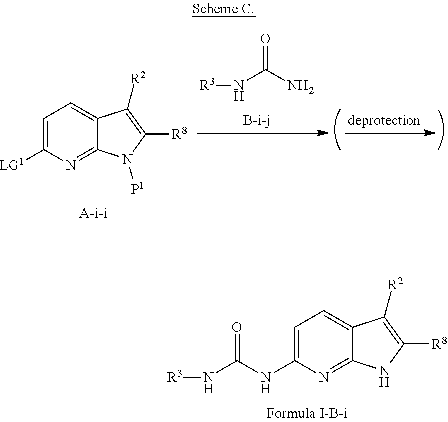

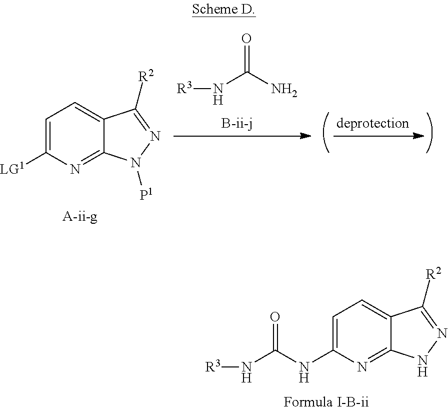

Alternatively, the addition of the cyclopropylcarboxamidyl or urea moiety as described in Scheme A (Part III), Scheme B (Part III), Scheme C and Scheme D may be substituted with the methods as described in Scheme E, Scheme F, Scheme G and Scheme H, respectively.

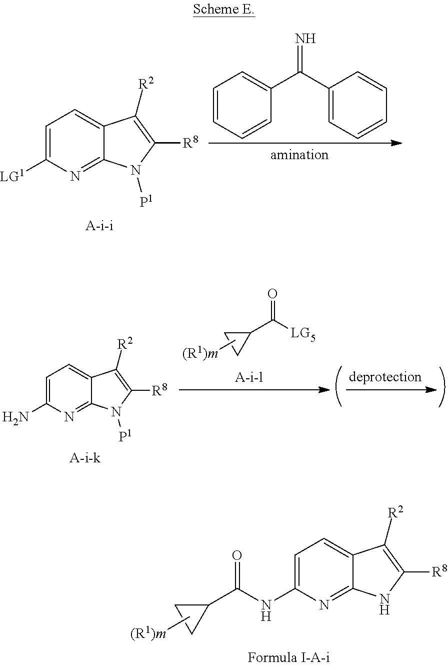

In Scheme E, the intermediate compounds of general formula A-i-i are aminated (such as with diphenylmethanimine) to give the compounds of general formula A-i-k. The resulting compounds of general formula A-i-k are subsequently reacted with a cyclopropanecarboxylic acid or derivative thereof A-i-l (wherein LG³ may be —OH, Cl—, —O—C₁-C₆ alkyl, etc.) to give the desired compounds of formula (I-A-i). Similarly, in Scheme F, the intermediate compounds of general formula A-ii-g are aminated (such as with diphenylmethanimine) to give the compounds of general formula A-ii-i. The resulting compounds of general formula A-ii-i are subsequently reacted with a cyclopropanecarboxylic acid or derivative thereof A-ii-j (wherein LG³ may be —OH, Cl—, —O—C₁-C₆ alkyl, etc.) to give the desired compounds of formula (I-A-ii).

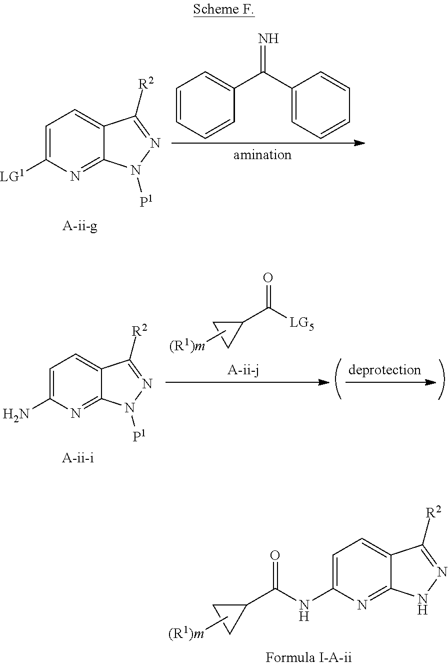

Compounds of general formula (I-B) may also be prepared from the compounds of general formulae A-i-k and A-ii-i as described in Schemes G and F. In Scheme G, the compounds of general formula A-i-k are reacted with carboxylic acid derivatives (e.g., phenyl carbonylchloridate and R³-containing free amines B-i-m in successive steps to give the desired urea compounds of general formula I-B-i. Similarly, in Scheme H, the compounds of general formula A-ii-i are reacted with carboxylic acid derivatives and R³-containing free amines B-ii-m in successive steps to give the desired urea compounds of general formula I-Bii.

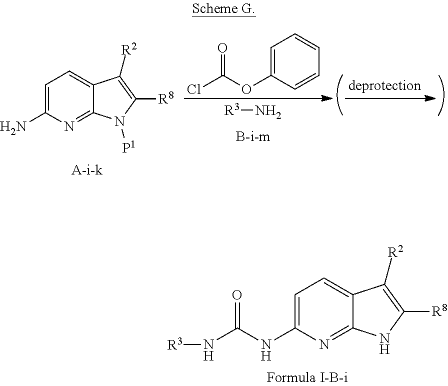

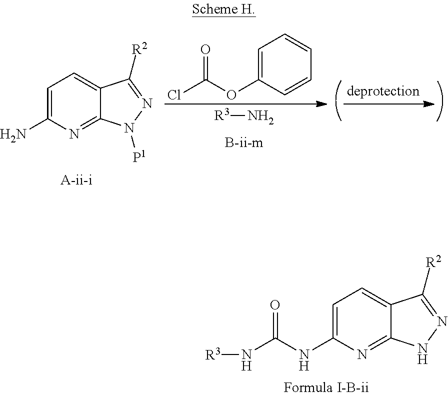

### IV. Pharmaceutical Compositions and Formulations

Any of the compounds described herein may be formulated as a pharmaceutically acceptable composition.

Pharmaceutical compositions of any of the compounds detailed herein are embraced by this disclosure. Thus, the present disclosure includes pharmaceutical compositions comprising a compound as detailed herein, or a pharmaceutically acceptable salt, solvate, hydrate, or co-crystal thereof, or a mixture of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound, or a pharmaceutically acceptable salt, solvate, hydrate, or co-crystal thereof, or a mixture of any of the foregoing, as detailed herein are provided, such as compositions of substantially pure compounds. In some embodiments, a composition containing a compound, or a pharmaceutically acceptable salt, solvate, hydrate, or co-crystal thereof, or a mixture of any of the foregoing, as detailed herein is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound selected from a compound of Table 1 intends a composition that contains no more than 35% impurity, wherein the impurity denotes a compound other than the compound of Table 1. In one variation, a composition of substantially pure compound, or a pharmaceutically acceptable salt, solvate, hydrate, or co-crystal thereof, or a mixture of any of the foregoing, is provided wherein the composition contains no more than 25% impurity. In another variation, a composition of substantially pure compound, or a pharmaceutically acceptable salt, solvate, hydrate, or co-crystal thereof, or a mixture of any of the foregoing, is provided wherein the composition contains or no more than 20% impurity. In still another variation, a composition of substantially pure compound, or a pharmaceutically acceptable salt, solvate, hydrate, or co-crystal thereof, or a mixture of any of the foregoing, is provided wherein the composition contains or no more than 10% impurity. In a further variation, a composition of substantially pure compound, or a pharmaceutically acceptable salt, solvate, hydrate, or co-crystal thereof, or a mixture of any of the foregoing, is provided wherein the composition contains no more than 5% impurity. In another variation, a composition of substantially pure compound, or a pharmaceutically acceptable salt, solvate, hydrate, or co-crystal thereof, or a mixture of any of the foregoing, is provided wherein the composition contains no more than 3% impurity. In still another variation, a composition of substantially pure compound, or a pharmaceutically acceptable salt, solvate, hydrate, or co-crystal thereof, or a mixture of any of the foregoing, is provided wherein the composition contains no more than 1% impurity. In a further variation, a composition of substantially pure compound, or a pharmaceutically acceptable salt, solvate, hydrate, or co-crystal thereof, or a mixture of any of the foregoing, is provided wherein the composition contains no more than 0.5% impurity. In yet other variations, a composition of substantially pure compound means that the composition contains no more than 15%, no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein. In some embodiments, the compounds and compositions as provided herein are sterile. Methods for sterilization known in the art may be suitable for any compounds or form thereof and compositions thereof as detailed herein.

A compound detailed herein, or a pharmaceutically acceptable salt, solvate, hydrate, or co-crystal thereof, or a mixture of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound, or a pharmaceutically acceptable salt, solvate, hydrate, or co-crystal thereof, or a mixture of any of the foregoing, may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a pharmaceutically acceptable salt, solvate, hydrate, or co-crystal thereof, or a mixture of any of the foregoing, can be used in the preparation of a formulation, such as a pharmaceutical formulation, by combining the compound or compounds, or a pharmaceutically acceptable salt, solvate, hydrate, or co-crystal thereof, or a mixture of any of the foregoing, with a pharmaceutically acceptable carrier. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, PA, 20th ed. (2000), which is incorporated herein by reference.

A compound detailed herein, or a pharmaceutically acceptable salt, solvate, hydrate, or co-crystal thereof, or a mixture of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds, or a pharmaceutically acceptable salt, solvate, hydrate, or co-crystal thereof, or a mixture of any of the foregoing, described herein can be formulated in a tablet in any dosage form described, for example, a compound as described herein, or a pharmaceutically acceptable salt, solvate, hydrate, or co-crystal thereof, or a mixture of any of the foregoing, can be formulated as a 10 mg tablet.

Compositions comprising a compound, or a pharmaceutically acceptable salt, solvate, hydrate, or co-crystal thereof, or a mixture of any of the foregoing, provided herein are also described. In one variation, the composition comprises a compound, or a pharmaceutically acceptable salt, solvate, hydrate, or co-crystal thereof, or a mixture of any of the foregoing, and a pharmaceutically acceptable carrier or excipient. In another variation, a composition of substantially pure compound, or a pharmaceutically acceptable salt, solvate, hydrate, or co-crystal thereof, or a mixture of any of the foregoing, is provided. In some embodiments, the composition is for use as a human or veterinary medicament. In some embodiments, the composition is for use in a method described herein. In some embodiments, the composition is for use in the treatment of a disease or disorder described herein.

Compositions formulated for co-administration of a compound provided herein and one or more additional pharmaceutical agents are also described. The co-administration can be simultaneous or sequential in any order. A compound provided herein may be formulated for co-administration with the one or more additional pharmaceutical agents in the same dosage form (e.g., single tablet or single i.v.) or separate dosage forms (e.g., two separate tablets, two separate i.v., or one tablet and one i.v.). Furthermore, co-administration can be, for example, 1) concurrent delivery, through the same route of delivery (e.g., tablet or i.v.), 2) sequential delivery on the same day, through the same route or different routes of delivery, or 3) delivery on different days, through the same route or different routes of delivery.

### V. Methods of Use

Compounds and compositions detailed herein, such as a pharmaceutical composition containing a compound of formula (I) or any variation thereof provided herein, or a pharmaceutically acceptable salt, solvate, hydrate, or co-crystal thereof, or a mixture of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is a method of inhibiting Bcr-Abl tyrosine kinase enzymatic activity, comprising contacting an effective amount of a compound or composition provided herein, to the Bcr-Abl tyrosine kinase. In some embodiments, provided herein is a method of inhibiting Bcr-Abl tyrosine kinase in a cell, comprising administering an effective amount of a compound or composition of the disclosure to the cell. In some embodiments, provided herein is a method of inhibiting Bcr-Abl tyrosine kinase in an individual in need thereof, comprising administering an effective amount of a compound or composition of the disclosure to the individual. In some variations, the compounds provided herein are selective for inhibiting Bcr-Abl tyrosine kinase. As such, in some embodiments, provided herein is a method of selectively inhibiting Bcr-Abl tyrosine kinase, as compared to other tyrosine kinases, including but not limited to c-KIT, FGFR, PDGFR, SRC, CSFR1, or VEGFR.

The compounds and compositions described herein may be used in a method of treating a disease or disorder mediated by Bcr-Abl tyrosine kinase activity. In some embodiments, the compound or composition is administered according to a dosage described herein.

In some embodiments, provided herein is a method for treating a disease or disorder mediated by Bcr-Abl tyrosine kinase activity comprising administering to an individual in need of treatment an effective amount of a compound of formula (I) or any variation thereof, or a pharmaceutically acceptable salt, solvate, hydrate, or co-crystal thereof, or a mixture of any of the foregoing. In some embodiments, the disease or disorder is a cancer mediated by Bcr-Abl tyrosine kinase activity. In some embodiments, the disease or disorder is chronic myeloid leukemia (CML), acute myeloid leukemia (AML), or acute lymphoblastic leukemia (ALL). In some embodiments, the disease or disorder is a cancer, such as leukemia. In some variations, the cancer is chronic myeloid leukemia (CML), Philadelphia-positive acute lymphoblastic leukemia (Ph+ALL), acute myelogenous leukemia (AML), or mixed phenotype acute leukemia.

In certain embodiments, the leukemia is chronic myeloid leukemia. Chronic myeloid leukemia may be characterized by the state of disease progression, as determined by blast cells. In still further embodiments, the chronic myeloid leukemia is chronic phase CML, accelerated phase CML, or blastic phase CML. In some embodiments, the chronic myeloid leukemia is refractory chronic myeloid leukemia.

In some embodiments, the disease or disorder mediated by Bcr-Abl tyrosine kinase activity is refractory or resistant to first-line treatment, second-line treatment, and/or third-line treatment. In certain embodiments, the condition mediated by Bcr-Abl tyrosine kinase activity is refractory or resistant to treatment with one or more Bcr-Abl tyrosine kinase inhibitors selected from the group consisting of imatinib, nilotinib, dasatinib, bafetinib, bosutinib, radotinib, asciminib, and ponatinib. First-line treatment as described herein includes the use of imatinib; second- and third-line treatments as described herein include the use of nilotinib, dasatinib, bafetinib, bosutinib, radotinib, asciminib, and/or ponatinib. In some variations of the foregoing, the chronic myeloid leukemia is refractory chronic myeloid leukemia.

Resistant subtypes of Bcr-Abl tyrosine kinase-mediated diseases or disorders may be associated with any number of Bcr-Abl dependent or Bcr-Abl independent resistance mechanisms. In some embodiments wherein the disease or disorder mediated by Bcr-Abl tyrosine kinase activity is refractory to treatment, the disease or disorder is characterized as being associated with one or more Bcr-Abl dependent resistance mechanisms. Bcr-Abl dependent resistance mechanisms include, but are not limited to, one or more point mutations at positions M244, L248, G250, G250, Q252, Q252, Y253, Y253, E255, E255, D276, F311, T315, T315, F317, F317, M343, M351, E355, F359, F359, V379, F382, L387, H396, H396, S417, E459, F486, or T315 in the Bcr-Abl tyrosine kinase. In certain variations, the refractory disease or disorder mediated by Bcr-Abl tyrosine kinase is associated with one or more specific point mutations in the Bcr-Abl tyrosine kinase selected from the group consisting of: M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I. In certain embodiments, the refractory disease or disorder mediated by Bcr-Abl tyrosine kinase is associated with a T315I mutation. In still further embodiments, the refractory disease or disorder mediated by Bcr-Abl tyrosine kinase is associated with a T315I mutation at the onset of treatment and I315M mutation following ponatinib. In other embodiments, the refractory disease or disorder mediated by Bcr-Abl tyrosine kinase is associated with one or more P-loop mutations (M244V, G250E, Q252H, Y253H/F, E255K/V).

In some embodiments, provided is a method for treating cancer in an individual in need thereof, comprising administering to the individual an effective amount of a compound of formula (I), or any variation thereof as described herein. In some embodiments, the cancer is leukemia. In some embodiments, the cancer is chronic myeloid leukemia (CML), Philadelphia-positive acute lymphoblastic leukemia (Ph+ALL), acute myelogenous leukemia (AML), or mixed phenotype acute leukemia. In some embodiments, the cancer is chronic myeloid leukemia (CML). In certain embodiments, the leukemia is chronic myeloid leukemia. In still further embodiments, the chronic myeloid leukemia is refractory chronic myeloid leukemia. In certain embodiments of the foregoing, the chronic myeloid leukemia is refractory chronic myeloid leukemia associated with a T315I mutation.

In one aspect, provided herein is a method of treating cancer in an individual in need thereof, wherein modulation of Bcr-Abl tyrosine kinase activity inhibits or ameliorates the pathology and/or symptomology of the cancer, comprising administering to the individual a therapeutically effective amount of a compound or composition provided herein. In one embodiment, provided herein is a method of treating cancer, wherein modulation of Bcr-Abl tyrosine kinase activity inhibits the pathology and/or symptomology of the cancer, in an individual, comprising administering to the individual a therapeutically effective amount of a compound or composition provided herein. In one embodiment, provided herein is a method of treating a cancer, wherein modulation of Bcr-Abl tyrosine kinase activity ameliorates the pathology and/or symptomology of the cancer, in an individual, comprising administering to the individual a therapeutically effective amount of a compound or composition provided herein.

In another aspect, provided herein is a method of preventing cancer, wherein modulation of Bcr-Abl tyrosine kinase activity prevents the pathology and/or symptomology of the cancer, in an individual, comprising administering to the individual a therapeutically effective amount of a compound or composition provided herein. In another aspect, provided herein is a method of delaying the onset and/or development of a cancer that is mediated by Bcr-Abl tyrosine kinase activity in an individual (such as a human) who is at risk for developing the cancer. It is appreciated that delayed development may encompass prevention in the event the individual does not develop the cancer.

In one aspect, provided herein is a method of delaying the onset and/or development of cancer in an individual in need thereof, comprising administering to the individual a therapeutically effective amount of a compound or composition provided herein. In some embodiments, the cancer is a leukemia. In certain embodiments, the cancer is chronic myeloid leukemia (CML), Philadelphia-positive acute lymphoblastic leukemia (Ph+ALL), acute myelogenous leukemia (AML), or mixed phenotype acute leukemia. In some embodiments, the cancer is chronic myeloid leukemia. In still further embodiments, the chronic myeloid leukemia is refractory chronic myeloid leukemia. In still yet other embodiments, the chronic myeloid leukemia is refractory chronic myeloid leukemia associated with a T315I mutation. In one aspect, provided herein is a method of delaying the onset and/or development of chronic myeloid leukemia in an individual in need thereof, comprising administering to the individual a therapeutically effective amount of a compound or composition provided herein. In one variation, provided herein is a method of delaying the onset and/or development of refractory chronic myeloid in an individual in need thereof, comprising administering to the individual a therapeutically effective amount of a compound or composition provided herein. In one variation, provided herein is a method of delaying the onset and/or development of refractory chronic myeloid leukemia associated with a T315I mutation in an individual in need thereof, comprising administering to the individual a therapeutically effective amount of a compound or composition provided herein.

In one aspect, provided herein is a compound of formula (I) or any variation thereof, or a pharmaceutically acceptable salt, solvate, hydrate, or co-crystal thereof, or a mixture of any of the foregoing, for use in therapy. In some embodiments, provided herein is a compound of formula (I) or any variation thereof, or a pharmaceutically acceptable salt, solvate, hydrate, or co-crystal thereof, or a mixture of any of the foregoing, or pharmaceutical composition comprising such compound, for use in the treatment of cancer. In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of chronic myeloid leukemia (CML), Philadelphia-positive acute lymphoblastic leukemia (Ph+ALL), acute myelogenous leukemia (AML), or mixed phenotype acute leukemia. In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of chronic myeloid leukemia (CML). In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of refractory chronic myeloid leukemia (CML). In some embodiments, provided is a compound of formula (I) or any variation thereof, or a pharmaceutically acceptable salt, solvate, hydrate, or co-crystal thereof, or a mixture of any of the foregoing, or a pharmaceutical composition comprising such compound, for use in the treatment of refractory chronic myeloid leukemia associated with a T315I mutation.

In another embodiment, provided herein is a compound of formula (I) or any variation thereof, or a pharmaceutically acceptable salt, solvate, hydrate, or co-crystal thereof, or a mixture of any of the foregoing, for use in the manufacture of a medicament for the treatment of cancer. In another embodiment, provided herein is a compound of formula (I) or any variation thereof, or a pharmaceutically acceptable salt, solvate, hydrate, or co-crystal thereof, or a mixture of any of the foregoing, for use in the manufacture of a medicament for the treatment of chronic myeloid leukemia (CML), Philadelphia-positive acute lymphoblastic leukemia (Ph+ALL), acute myelogenous leukemia (AML), or mixed phenotype acute leukemia. In some embodiments, the medicament is for the treatment of chronic myeloid leukemia. In some embodiments, the medicament is for the treatment of refractory chronic myeloid leukemia. In some embodiments, the medicament is for the treatment of refractory chronic myeloid leukemia associated with a T315I mutation.

In some embodiments, the individual is a mammal. In some embodiments, the individual is a primate, dog, cat, rabbit, or rodent. In some embodiments, the individual is a primate. In some embodiments, the individual is a human. In some embodiments, the human is at least about or is about any of 18, 21, 30, 50, 60, 65, 70, 75, 80, or 85 years old. In some embodiments, the human is a child. In some embodiments, the human is less than about or about any of 21, 18, 15, 10, 5, 4, 3, 2, or 1 years old.

In some embodiments, the method further comprises administering one or more additional pharmaceutical agents. In some embodiments, the method further comprises administering radiation. In some embodiments, the method further comprises administering one or more additional pharmaceutical agents, including anti-microtubular therapies (e.g. paclitaxel, vincristine), topoisomerase inhibitors (e.g. adriamycin), alylating agents (e.g. busulfan, cyclophosphamide), nucleotide synthesis inhibitors (hyroxyurea), DNA synthesis inhibitors (e.g. cytarabine), protein synthesis inhibitors (e.g. omacetaxine), developmental signaling pathway inhibitors (e.g. sonidegib, Hedgehog pathway), pro-apoptotic agents (e.g. venetoclax), Abl myristoyl-pocket binding inhibitors (e.g. asciminib), MEK1/2 inhibitors (e.g. trametinib, binimetinib), AKT inhibitors (e.g. ipatasertib), PI3K inhibitors (e.g. apelisib) and radiation.

### VI. Dosing and Method of Administration

The dose of a compound described herein, or a pharmaceutically acceptable salt, solvate, hydrate, or co-crystal thereof, or a mixture of any of the foregoing, administered to an individual (such as a human) may vary with the particular compound or salt thereof, the method of administration, and the particular cancer, such as type and stage of cancer, being treated. In some embodiments, the amount of the compound, or a pharmaceutically acceptable salt, solvate, hydrate, or co-crystal thereof, or a mixture of any of the foregoing, is a therapeutically effective amount.

The compounds provided herein, or a pharmaceutically acceptable salt, solvate, hydrate, or co-crystal thereof, or a mixture of any of the foregoing, may be administered to an individual via various routes, including, e.g., intravenous, intramuscular, subcutaneous, oral, and transdermal.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein, or a pharmaceutically acceptable salt, solvate, hydrate, or co-crystal thereof, or a mixture of any of the foregoing, and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

### VII. Articles of Manufacture and Kits

The present disclosure further provides articles of manufacture comprising a compound described herein, or a pharmaceutically acceptable salt, solvate, hydrate, or co-crystal thereof, or a mixture of any of the foregoing, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein, or a pharmaceutically acceptable salt, solvate, hydrate, or co-crystal thereof, or a mixture of any of the foregoing, thereof. The kits may be used for any one or more of the uses described herein, and, accordingly, may contain instructions for the treatment of any disease or described herein, for example for the treatment of cancer, including chronic myeloid leukemia (CML), Philadelphia-positive acute lymphoblastic leukemia (Ph+ALL), acute myelogenous leukemia (AML), or mixed phenotype acute leukemia. In some embodiments, the cancer is chronic myeloid leukemia. In some embodiments, the cancer is refractory chronic myeloid leukemia. In certain embodiments of the foregoing, the cancer is refractory chronic myeloid leukemia associated with a T315I mutation.

The kits optionally further comprise a container comprising one or more additional pharmaceutical agents and which kits further comprise instructions on or in the package insert for treating the subject with an effective amount of the one or more additional pharmaceutical agents.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

It is understood that the present disclosure has been made only by way of example, and that numerous changes in the combination and arrangement of parts can be resorted to by those skilled in the art without departing from the spirit and scope of present disclosure.

### Synthetic Examples

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following: ACN: acetonitrile; Brettphos: 2-(dicyclohexylphosphino)3,6-dimethoxy-2′,4′,6′-triisopropyl-1,1′-biphenyl; dppf: 1,1′-ferrocenediyl-bis(diphenylphosphine); DCM: dichloromethane; DIAD: diisopropylazodicarboxylate; DIEA: N,N-diisopropylethylamine; DMAP: 4-dimethylaminopyridine; DMF: dimethylformamide; DMF-DMA: N,N-dimethylformamide dimethylacetal; DMSO: dimethyl sulfoxide; EDA; ethylenediamine; EtOAc: ethyl acetate; EtOH: ethanol or ethyl alcohol; F-TEDA-BF₄: 1-Chloromethyl-4-fluoro-1,4-diazoniabicyclo[2.2.2]octane bis(tetrafluoroborate); ¹H NMR: proton nuclear magnetic resonance; HATU: 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium; LCMS: liquid chromatography-mass spectrometry; LDA: lithium diisopropylamide; LiHMDS: lithium hexamethyldisilazide; MeOH: methanol or methyl alcohol; NBS: N-bromosuccinimide; NIS: N-iodosuccinimide; NNMP: N-methyl-2-pyrrolidone; OAc: acetate; Py: pyridine; TBAB: tetra-n-butylammonium bromide; TBAF: tetra-n-butylammonium fluoride; TEA: triethylamine; TFA: trifluoroacetic acid; THF: tetrahydrofuran; and TLC: thin-layer chromatography.

### Example S1. Compound 1

### Step 1: Synthesis of 3-bromo-6-chloro-1H-pyrrolo[2,3-b]pyridine

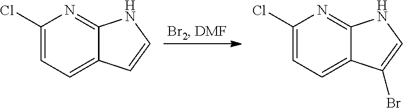

To a solution of 6-chloro-1H-pyrrolo[2,3-b]pyridine (500.0 mg, 3.28 mmol) in DMF (5.0 mL) was added Br₂ (549.9 mg, 3.44 mmol) dropwise at 0° C. under N₂. The resulting mixture was stirred at 0° C. for 3 h under N₂. After the reaction was completed, the reaction was quenched with aq·NaHSO₃ at 10° C. The pH value of the mixture was adjusted to 8 with aq·NaHCO₃. The resulting mixture was filtered. The solid was washed with H₂O, collected and dried to afford 3-bromo-6-chloro-1H-pyrrolo[2,3-b]pyridine (620.0 mg, crude) as a white solid. LCMS (ESI, m/z): [M+H]⁺=230.9.

### Step 2: Synthesis of tert-butyl 3-bromo-6-chloro-1H-pyrrolo[2,3-b]pyridine-1-carboxylate

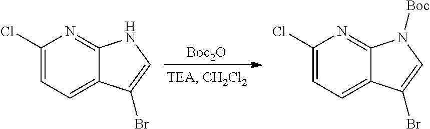 [SMILES: "Boc2O" ; Clc1ccc2c(Br)c[nH]c2n1 ; CC(C)(C)OC(=O)n1cc(Br)c2ccc(Cl)nc21]

To a solution of 3-bromo-6-chloro-1H-pyrrolo[2,3-b]pyridine (620.0 mg, crude) in CH₂Cl₂ (10.0 mL) was added Boc₂O (759.9 mg, 3.48 mmol) and TEA (758.9 mg, 7.50 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (85/15, v/v) to afford tert-butyl 3-bromo-6-chloro-1H-pyrrolo[2,3-b]pyridine-1-carboxylate (850.0 mg, 96%) as a white solid. LCMS (ESI, m/z): [M+H]⁺=331.0.

### Step 3: Synthesis of 6-chloro-3-(2-methoxyphenyl)-1H-pyrrolo[2,3-b]pyridine

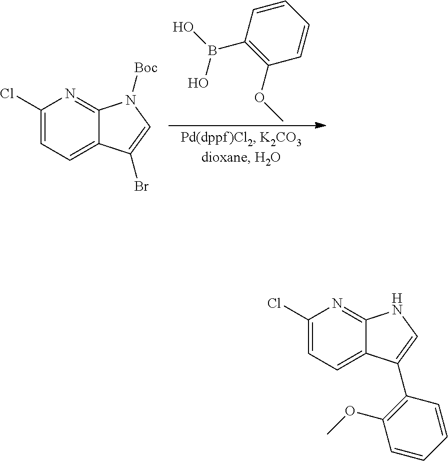 [SMILES: CC(C)(C)OC(=O)n1cc(Br)c2ccc(Cl)nc21 ; COc1ccccc1B(O)O ; COc1ccccc1-c1c[nH]c2nc(Cl)ccc12]

To a solution of tert-butyl 3-bromo-6-chloro-1H-pyrrolo[2,3-b]pyridine-1-carboxylate (500.0 mg, 1.51 mmol) in 1,4-dioxane/H₂O (20.0/2.0 mL) was added (2-methoxyphenyl)boronic acid (275.0 mg, 1.81 mmol), K₂CO₃ (625.2 mg, 4.52 mmol) and Pd(dppf)Cl₂ (110.3 mg, 0.15 mmol). The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (83/17, v/v) to afford 6-chloro-3-(2-methoxyphenyl)-1H-pyrrolo[2,3-b]pyridine (190.0 mg, 49%) as a white solid. LCMS (ESI, m/z): [M+H]⁺=259.1.

### Step 4: Synthesis of N-(3-(2-methoxyphenyl)-1H-pyrrolo[2,3-b]pyridin-6-yl)cyclopropanecarboxamide (Compound 1)

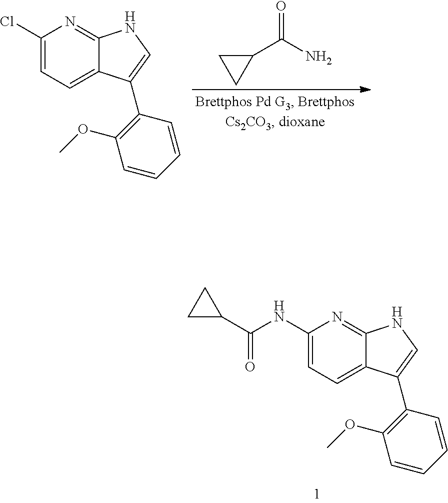 [SMILES: COc1ccccc1-c1c[nH]c2nc(Cl)ccc12 ; NC(=O)C1CC1 ; COc1ccccc1-c1c[nH]c2nc(NC(=O)C3CC3)ccc12]

To a stirred solution of 6-chloro-3-(2-methoxyphenyl)-1H-pyrrolo[2,3-b]pyridine (140.0 mg, 0.54 mmol) in 1,4-dioxane (15.0 mL) was added cyclopropanecarboxamide (184.2 mg, 2.17 mmol), BrettPhos (58.1 mg, 0.11 mmol), Cs₂CO₃ (529.0 mg, 1.62 mmol) and BrettPhos Pd G3 (49.1 mg, 0.05 mmol). The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (95/5, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% B to 57% B in 7 min; 254 nm) to afford N-(3-(2-methoxyphenyl)-1H-pyrrolo[2,3-b]pyridin-6-yl)cyclopropanecarboxamide (14.6 mg, 9%) as a white solid. LCMS (ESI, m/z): [M+H]⁺=308.2. ¹H NMR (300 MHz, DMSO-d₆): δ 11.55 (s, 1H), 10.58 (s, 1H), 7.98-7.87 (m, 2H), 7.56-7.52 (m, 2H), 7.35-7.19 (m, 1H), 7.11 (d, J=8.1 Hz, 1H), 7.07-6.92 (m, 1H), 3.81 (s, 3H), 2.11-1.98 (m, 1H), 0.97-0.73 (m, 4H).

### Example S2. Compound 2

### Step 1: Synthesis of 6-chloro-3-(2-methoxyphenyl)-1-((2-(trimethylsilyl)ethoxy)methyl)-1H-pyrrolo[2,3-b]pyridine

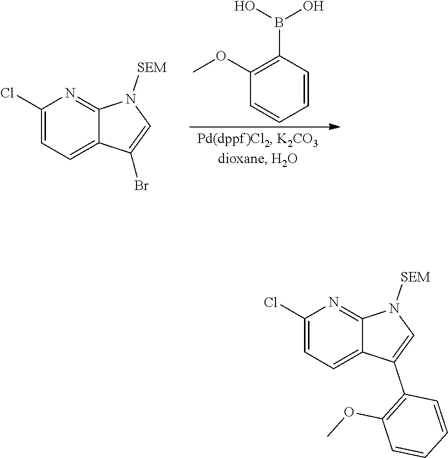 [SMILES: C[Si](C)(C)CCOCn1cc(Br)c2ccc(Cl)nc21 ; COc1ccccc1B(O)O ; COc1ccccc1-c1cn(COCC[Si](C)(C)C)c2nc(Cl)ccc12]

To a solution of 3-bromo-6-chloro-1-((2-(trimethylsilyl)ethoxy)methyl)-1H-pyrrolo[2,3-b]pyridine (600.0 mg, 1.66 mmol) in 1,4-dioxane/H₂O (5.0/1.0 mL) was added (2-methoxyphenyl)boronic acid (302.5 mg, 1.99 mmol), K₂CO₃ (687.7 mg, 4.98 mmol) and Pd(dppf)Cl₂ (121.4 mg, 0.17 mmol) at room temperature under N₂. The resulting mixture was stirred at 80° C. for 3 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2-methoxyphenyl)-1-((2-(trimethylsilyl)ethoxy)methyl)-1H-pyrrolo[2,3-b]pyridine (390.0 mg, 60%) as a yellow oil. LCMS (ESI, m/z): [M+H]⁺=389.1.

### Step 2: Synthesis of (1R,2R)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide

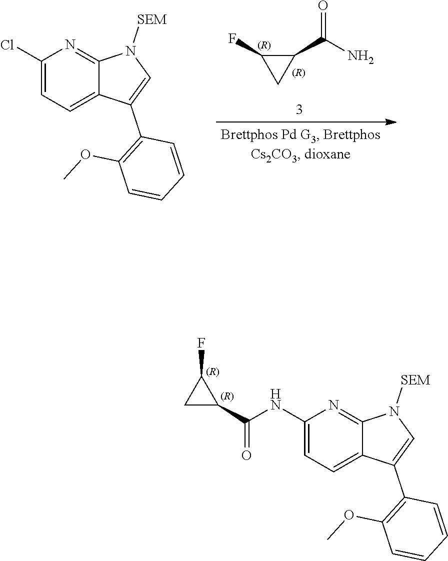

To a solution of 6-chloro-3-(2-methoxyphenyl)-1-((2-(trimethylsilyl)ethoxy)methyl)-1H-pyrrolo[2,3-b]pyridine (190.0 mg, 0.49 mmol) in 1,4-dioxane (10.0 mL) was added (1R,2R)-2-fluorocyclopropane-1-carboxamide (251.8 mg, 2.44 mmol), BrettPhos (52.4 mg, 0.10 mmol), Cs₂CO₃ (477.4 mg, 1.47 mmol) and BrettPhos Pd G3 (44.3 mg, 0.05 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (67/33, v/v) to afford (1R,2R)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide (110.0 mg, 49%) as a yellow oil. LCMS (ESI, m/z): [M+H]⁺=456.2.

### Step 3: Synthesis of (1R,2R)-2-fluoro-N-(3-(2-methoxyphenyl)-1H-pyrrolo[2,3-b]pyridin-6-yl)cyclopropane-1-carboxamide (Compound 2)

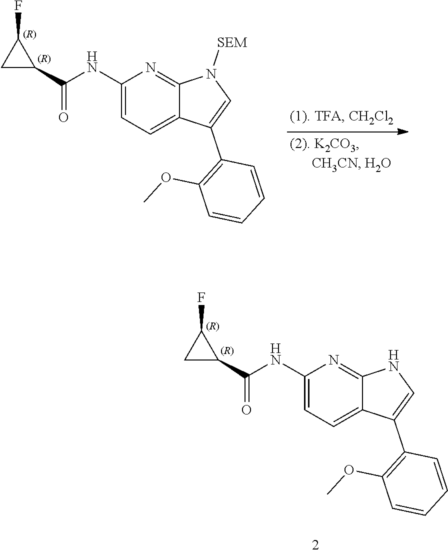

To a solution of (1R,2R)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide (100.0 mg, 0.22 mmol) in CH₂Cl₂ (5.0 mL) was added TFA (5.0 mL) at room temperature. The resulting mixture was stirred at room temperature for 3 h. The reaction mixture was evaporated in vacuo. The residue was re-dissolved in ACN/H₂O (5.0/1.0 mL). Then K₂CO₃ (380.7 mg, 2.76 mmol) was added to the mixture. The mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 39% B to 52% B in 7 min; 254 nm) to afford (1R,2R)-2-fluoro-N-(3-(2-methoxyphenyl)-1H-pyrrolo[2,3-b]pyridin-6-yl)cyclopropane-1-carboxamide (14.0 mg, 20%) as a white solid. LCMS (ESI, m/z): [M+H]⁺=326.1. ¹H NMR (300 MHz, DMSO-d₆): δ 11.58 (s, 1H), 10.64 (s, 1H), 8.00 (d, J=8.7 Hz, 1H), 7.90 (d, J=8.7 Hz, 1H), 7.58-7.53 (m, 2H), 7.31-7.25 (m, 1H), 7.12 (d, J=7.5 Hz, 1H), 7.06-7.00 (m, 1H), 5.10-4.78 (m, 1H), 3.82 (s, 3H), 2.30-2.15 (m, 1H), 1.75-1.67 (m, 1H), 1.25-1.08 (m, 1H).

### Example S3. Compound 3

### Step 1: Synthesis of (1S,2S)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide

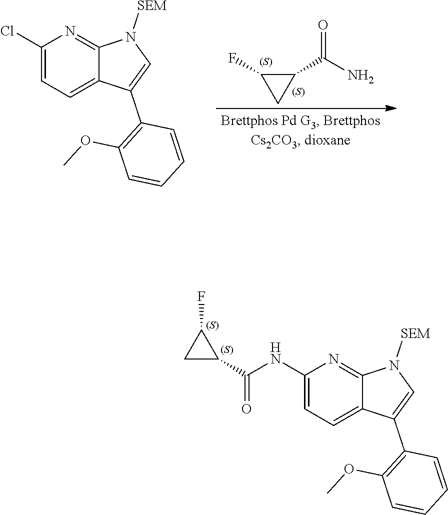

To a solution of 6-chloro-3-(2-methoxyphenyl)-1-((2-(trimethylsilyl)ethoxy)methyl)-1H-pyrrolo[2,3-b]pyridine (190.0 mg, 0.49 mmol) in 1,4-dioxane (10.0 mL) was added (1S,2S)-2-fluorocyclopropane-1-carboxamide (251.8 mg, 2.44 mmol), BrettPhos (52.4 mg, 0.10 mmol), Cs₂CO₃ (477.4 mg, 1.47 mmol) and BrettPhos Pd G3 (44.3 mg, 0.05 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (67/33, v/v) to afford (1S,2S)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide (100.0 mg, 45%) as a yellow oil. LCMS (ESI, m/z): [M+H]⁺=456.2.

### Step 2: Synthesis of (1S,2S)-2-fluoro-N-(3-(2-methoxyphenyl)-1H-pyrrolo[2,3-b]pyridin-6-yl)cyclopropane-1-carboxamide (Compound 3)

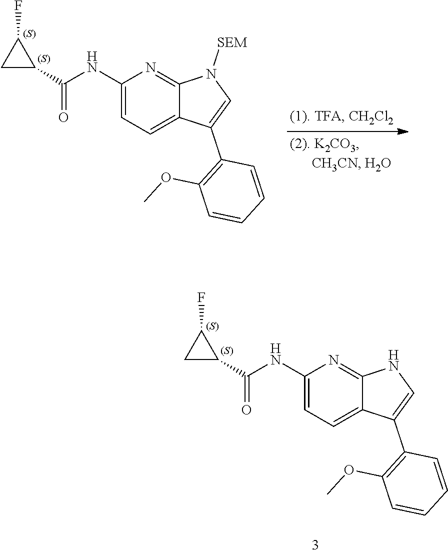

To a solution of (1S,2S)-2-fluoro-N-(3-(2-methoxyphenyl)-1-((2-(trimethylsilyl)ethoxy)methyl)-1H-pyrrolo[2,3-b]pyridin-6-yl)cyclopropane-1-carboxamide (100.0 mg, 0.22 mmol) in CH₂Cl₂ (5.0 mL) was added TFA (5.0 mL) at room temperature. The resulting mixture was stirred at room temperature for 3 h. The reaction mixture was evaporated in vacuo. The residue was re-dissolved in ACN/H₂O (5.0/1.0 mL). Then K₂CO₃ (380.7 mg, 2.76 mmol) was added to the mixture. The reaction mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 39% B to 52% B in 7 min; 254 nm) to afford (1S,2S)-2-fluoro-N-(3-(2-methoxyphenyl)-1H-pyrrolo[2,3-b]pyridin-6-yl)cyclopropane-1-carboxamide (10.6 mg, 14%) as a light pink solid. LCMS (ESI, m/z): [M+H]⁺=326.2. ¹H NMR (300 MHz, DMSO-d₆): δ 11.57 (s, 1H), 10.63 (s, 1H), 8.00 (d, J=8.7 Hz, 1H), 7.89 (d, J=8.7 Hz, 1H), 7.57-7.52 (m, 2H), 7.30-7.24 (m, 1H), 7.12-7.09 (m, 1H), 7.05-7.00 (m, 1H), 5.05-4.80 (m, 1H), 3.82 (s, 3H), 2.27-2.21 (m, 1H), 1.70-1.61 (m, 1H), 1.19-1.12 (m, 1H).

### Example S4. Compound 4

### Step 1: Synthesis of 6-chloro-3-(2-methylphenyl)-1-[[2-(trimethylsilyl)ethoxy]methyl]pyrrolo[2,3-b]pyridine

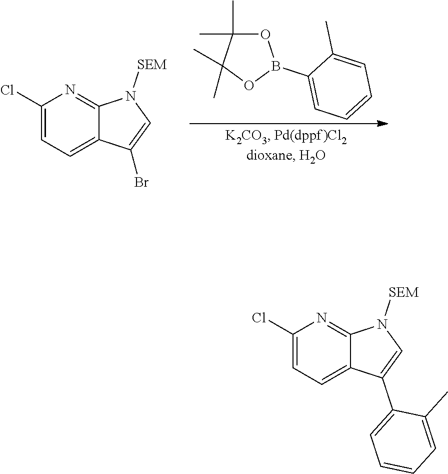 [SMILES: C[Si](C)(C)CCOCn1cc(Br)c2ccc(Cl)nc21 ; Cc1ccccc1B1OC(C)(C)C(C)(C)O1 ; Cc1ccccc1-c1cn(COCC[Si](C)(C)C)c2nc(Cl)ccc12]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 4,4,5,5-tetramethyl-2-(2-methylphenyl)-1,3,2-dioxaborolane (217.0 mg, 0.99 mmol), K₂CO₃ (343.9 mg, 2.49 mmol) and Pd(dppf)Cl₂ (121.4 mg, 0.17 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 6-chloro-3-(2-methylphenyl)-1-[[2-(trimethylsilyl)ethoxy]methyl]pyrrolo[2,3-b]pyridine (150.0 mg, 49%) as a white solid. LCMS (ESI, m/z): [M+H]⁺=373.1.

### Step 2: Synthesis of N-[3-(2-methylphenyl)-1-[[2-(trimethylsilyl)ethoxy]methyl]pyrrolo[2,3-b]pyridin-6-yl]cyclopropanecarboxamide

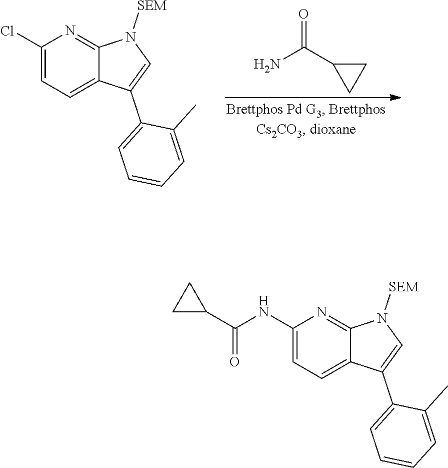 [SMILES: Cc1ccccc1-c1cn(COCC[Si](C)(C)C)c2nc(Cl)ccc12 ; NC(=O)C1CC1 ; Cc1ccccc1-c1cn(COCC[Si](C)(C)C)c2nc(NC(=O)C3CC3)ccc12]

To a solution of 6-chloro-3-(2-methylphenyl)-1-[[2-(trimethylsilyl)ethoxy]methyl]pyrrolo[2,3-b]pyridine (120.0 mg, 0.32 mmol) in dioxane (8.0 mL) was added cyclopropanecarboxamide (136.9 mg, 1.60 mmol), BrettPhos (69.0 mg, 0.13 mmol), Cs₂CO₃ (314.5 mg, 0.97 mmol) and BrettPhos Pd G3 (58.3 mg, 0.06 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford N-[3-(2-methylphenyl)-1-[[2-(trimethylsilyl)ethoxy]methyl]pyrrolo[2,3-b]pyridin-6-yl]cyclopropanecarboxamide (120.0 mg, 88%) as a white solid. LCMS (ESI, m/z): [M+H]⁺=422.2.

### Step 3: Synthesis of N-[3-(2-methylphenyl)-1H-pyrrolo[2,3-b]pyridin-6-yl]cyclopropanecarboxamide (Compound 4)

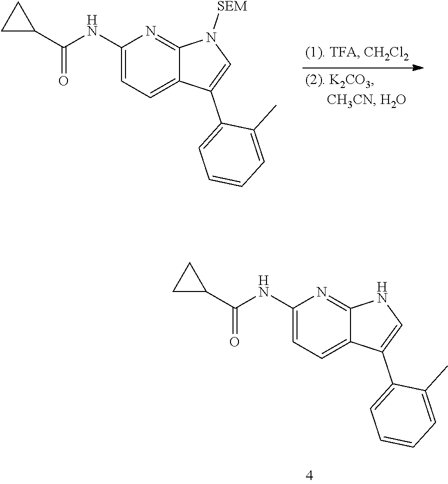 [SMILES: Cc1ccccc1-c1cn(COCC[Si](C)(C)C)c2nc(NC(=O)C3CC3)ccc12 ; Cc1ccccc1-c1c[nH]c2nc(NC(=O)C3CC3)ccc12]

To a solution of N-[3-(2-methylphenyl)-1-[[2-(trimethylsilyl)ethoxy]methyl]pyrrolo[2,3-b]pyridin-6-yl]cyclopropanecarboxamide (100.0 mg, 0.24 mmol) in DCM (3.0 mL) was added TFA (3.0 mL) at room temperature. The resulting mixture was stirred at room temperature for 3 h. The resulting mixture was concentrated under reduced pressure. The residue was re-dissolved in ACN/H₂O (5.0/1.0 mL). Then K₂CO₃ (163.6 mg, 1.18 mmol) was added to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 48% B to 58% B in 7 min; 254 nm) to afford N-[3-(2-methylphenyl)-1H-pyrrolo[2,3-b]pyridin-6-yl]cyclopropanecarboxamide (13.6 mg, 20%) as a white solid. LCMS (ESI, m/z): [M+H]⁺=292.1. ¹H NMR (400 MHz, DMSO-d₆): δ 11.59 (s, 1H), 11.60 (s, 1H), 7.89 (d, J=8.4 Hz, 1H), 7.78 (s, 1H), 7.42 (d, J=2.4 Hz, 1H), 7.37-7.31 (m, 2H), 7.25-7.22 (m, 2H), 2.31 (s, 3H), 2.06-2.02 (m, 1H), 0.82-0.80 (m, 4H).

### Example S5. Compound 5

### Step 1: Synthesis of 6-chloro-3-(2-fluorophenyl)-1-[[2-(trimethylsilyl)ethoxy]methyl]pyrrolo[2,3-b]pyridine

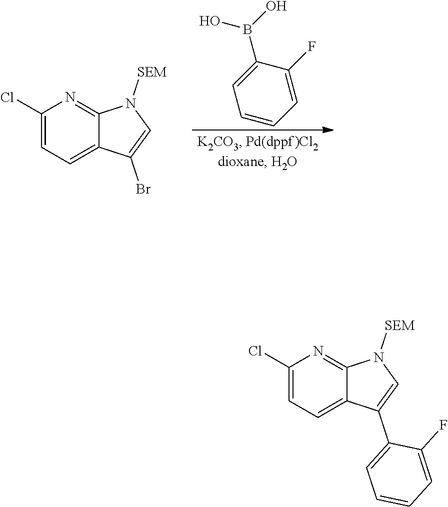 [SMILES: C[Si](C)(C)CCOCn1cc(Br)c2ccc(Cl)nc21 ; OB(O)c1ccccc1F ; C[Si](C)(C)CCOCn1cc(-c2ccccc2F)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 2-fluorophenylboronic acid (139.2 mg, 1.01 mmol), K₂CO₃ (343.8 mg, 2.49 mmol) and Pd(dppf)Cl₂ (60.7 mg, 0.08 mmol) at room temperature under N₂. The resulting mixture was stirred at 80° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (93/7, v/v) to afford 6-chloro-3-(2-fluorophenyl)-1-[[2-(trimethylsilyl)ethoxy]methyl]pyrrolo[2,3-b]pyridine (220.0 mg, 70%) as a yellow oil. LCMS (ESI, m/z): [M+H]⁺=377.1.

### Step 2: Synthesis of N-[3-(2-fluorophenyl)-1-[[2-(trimethylsilyl)ethoxy]methyl]pyrrolo[2,3-b]pyridin-6-yl]cyclopropanecarboxamide

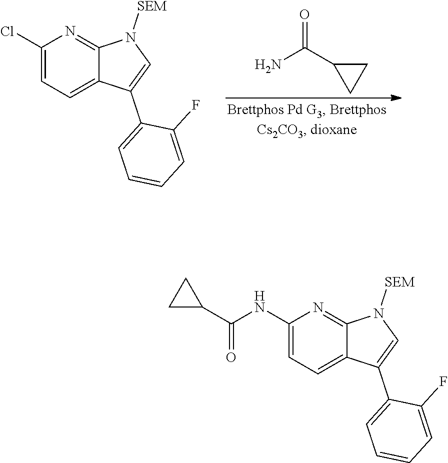 [SMILES: C[Si](C)(C)CCOCn1cc(-c2ccccc2F)c2ccc(Cl)nc21 ; NC(=O)C1CC1 ; C[Si](C)(C)CCOCn1cc(-c2ccccc2F)c2ccc(NC(=O)C3CC3)nc21]

To a solution of 6-chloro-3-(2-fluorophenyl)-1-[[2-(trimethylsilyl)ethoxy]methyl]pyrrolo[2,3-b]pyridine (170.0 mg, 0.45 mmol) in 1,4-dioxane (5.0 mL) was added cyclopropanecarboxamide (115.1 mg, 1.35 mmol), Cs₂CO₃ (440.8 mg, 1.35 mmol), Brettphos (48.42 mg, 0.09 mmol) and Brettphos Pd G3 (40.9 mg, 0.04 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (83/17, v/v) to afford N-[3-(2-fluorophenyl)-1-[[2-(trimethylsilyl)ethoxy]methyl]pyrrolo[2,3-b]pyridin-6-yl]cyclopropanecarboxamide (210.0 mg, 62%) as a yellow solid. LCMS (ESI, m/z): [M+H]⁺=426.1.

### Step 3: Synthesis of N-[3-(2-fluorophenyl)-1H-pyrrolo[2,3-b]pyridin-6-yl]cyclopropanecarboxamide (Compound 5)

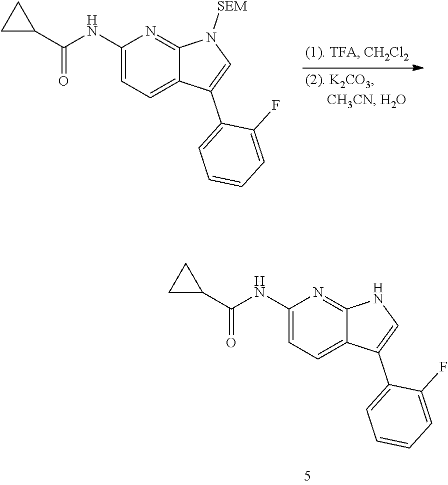 [SMILES: C[Si](C)(C)CCOCn1cc(-c2ccccc2F)c2ccc(NC(=O)C3CC3)nc21 ; O=C(Nc1ccc2c(-c3ccccc3F)c[nH]c2n1)C1CC1]

To a solution of N-[3-(2-fluorophenyl)-1-[[2-(trimethylsilyl)ethoxy]methyl]pyrrolo[2,3-b]pyridin-6-yl]cyclopropanecarboxamide (150.0 mg, 0.35 mmol) in CH₂Cl₂ (5.0 mL) was added TFA (5.0 mL). The resulting mixture was stirred at room temperature for 1 h. The reaction mixture was evaporated in vacuo. The residue was re-dissolved in ACN/H₂O (5.0/1.0 mL). Then K₂CO₃ (487.1 mg, 3.53 mmol) was added to the mixture. The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 38% B to 68% B in 7 min; 254 nm) to afford N-[3-(2-fluorophenyl)-1H-pyrrolo[2,3-b]pyridin-6-yl]cyclopropanecarboxamide (54.7 mg, 52%) as a white solid. LCMS (ESI, m/z): [M+H]⁺=296.2. ¹H NMR (300 MHz, DMSO-d₆): δ 11.80 (s, 1H), 10.65 (s, 1H), 8.08-8.05 (m, 1H), 7.95 (d, J=8.7 Hz, 1H), 7.76-7.70 (m, 1H), 7.66 (s, 1H), 7.34-7.26 (m, 3H), 2.08-2.03 (m, 1H), 0.86-0.76 (m, 4H).

### Example S6. Compound 6

### Step 1: Synthesis of 6-chloro-3-(2-chlorophenyl)-1-[[2-(trimethylsilyl)ethoxy]methyl]pyrrolo[2,3-b]pyridine

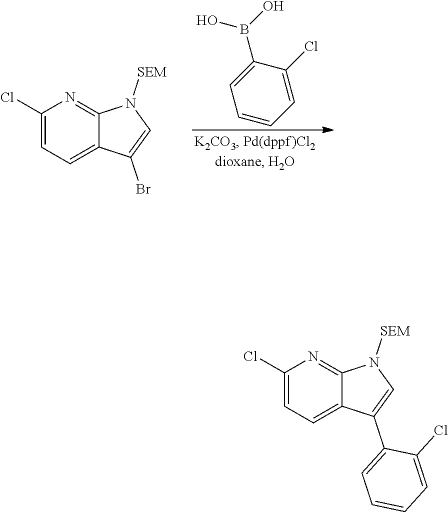 [SMILES: C[Si](C)(C)CCOCn1cc(Br)c2ccc(Cl)nc21 ; OB(O)c1ccccc1Cl ; C[Si](C)(C)CCOCn1cc(-c2ccccc2Cl)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 2-chlorophenylboronic acid (155.6 mg, 0.99 mmol), K₂CO₃ (343.9 mg, 2.49 mmol) and Pd(dppf)Cl₂ (60.7 mg, 0.08 mmol) at room temperature under N₂. The resulting mixture was stirred at 80° C. for 3 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2-chlorophenyl)-1-[[2-(trimethylsilyl)ethoxy]methyl]pyrrolo[2,3-b]pyridine (210.0 mg, 64%) as a yellow oil. LCMS (ESI, m/z): [M+H]⁺=393.1.

### Step 2: Synthesis of N-[3-(2-chlorophenyl)-1-[[2-(trimethylsilyl)ethoxy]methyl]pyrrolo[2,3-b]pyridin-6-yl]cyclopropanecarboxamide

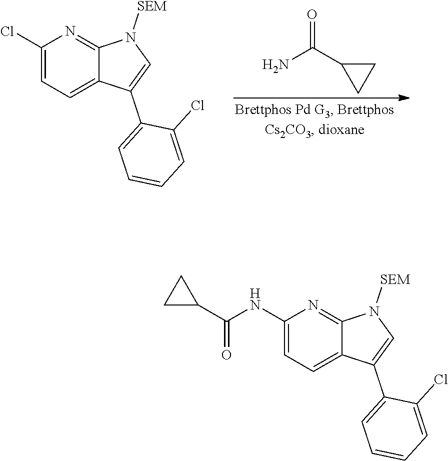 [SMILES: C[Si](C)(C)CCOCn1cc(-c2ccccc2Cl)c2ccc(Cl)nc21 ; NC(=O)C1CC1 ; C[Si](C)(C)CCOCn1cc(-c2ccccc2Cl)c2ccc(NC(=O)C3CC3)nc21]

To a solution of 6-chloro-3-(2-chlorophenyl)-1-[[2-(trimethylsilyl)ethoxy]methyl]pyrrolo[2,3-b]pyridine (110.0 mg, 0.28 mmol) in 1,4-dioxane (3.0 mL) was added cyclopropanecarboxamide (23.8 mg, 0.28 mmol), BrettPhos (30.0 mg, 0.06 mmol), Cs₂CO₃ (273.3 mg, 0.38 mmol) and BrettPhos Pd G3 (25.4 mg, 0.03 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford N-[3-(2-chlorophenyl)-1-[[2-(trimethylsilyl)ethoxy]methyl]pyrrolo[2,3-b]pyridin-6-yl]cyclopropanecarboxamide (105.0 mg, 85%) as a yellow oil. LCMS (ESI, m/z): [M+H]⁺=442.2.

### Step 3: Synthesis of N-[3-(2-chlorophenyl)-1H-pyrrolo[2,3-b]pyridin-6-yl]cyclopropanecarboxamide (Compound 6)

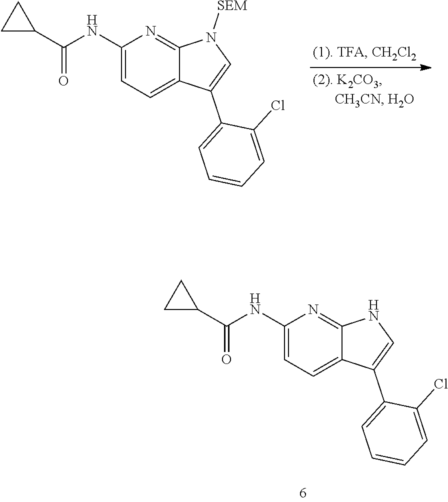 [SMILES: C[Si](C)(C)CCOCn1cc(-c2ccccc2Cl)c2ccc(NC(=O)C3CC3)nc21 ; O=C(Nc1ccc2c(-c3ccccc3Cl)c[nH]c2n1)C1CC1]

To a solution of N-[3-(2-chlorophenyl)-1-[[2-(trimethylsilyl)ethoxy]methyl]pyrrolo[2,3-b]pyridin-6-yl]cyclopropanecarboxamide (105.0 mg, 0.24 mmol) in CH₂Cl₂ (5.0 mL) was added TFA (5.0 mL) at room temperature. The resulting mixture was stirred at room temperature for 3 h. The mixture was evaporated in vacuo. The residue was re-dissolved with ACN/H₂O (5.0/3.0 mL). Then K₂CO₃ (328.3 mg, 2.38 mmol) was added to the mixture. The mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by Prep-HPLC with the following conditions (Column: XBridge Prep Phenyl OBD Column, 5 um, 19×250 mm; Mobile Phase A: Water (0.05% FA), Mobile Phase B: MeOH—HPLC; Flow rate: 25 mL/min; Gradient: 54% B to 70% B in 10 min; 254 nm) to afford N-[3-(2-chlorophenyl)-1H-pyrrolo[2,3-b]pyridin-6-yl]cyclopropanecarboxamide (8.1 mg, 10%) as a white solid. LCMS (ESI, m/z): [M+H]⁺=312.1. ¹H NMR (300 MHz, DMSO-d₆): δ 11.79 (s, 1H), 10.64 (s, 1H), 7.98-7.91 (m, 1H), 7.87-7.84 (m, 1H), 7.60-7.57 (m, 3H), 7.49-7.30 (m, 2H), 2.10-2.00 (m, 1H), 0.83-0.79 (m, 4H).

### Example S7. Compound 7

### Step 1: Synthesis of 6-chloro-3-[2-(trifluoromethyl)phenyl]-1-[[2-(trimethylsilyl)ethoxy]methyl]pyrrolo[2,3-b]pyridine

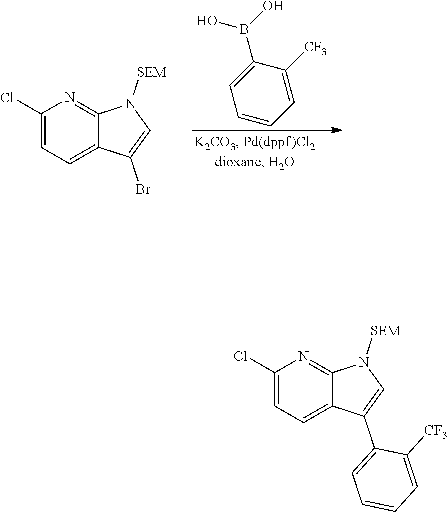 [SMILES: C[Si](C)(C)CCOCn1cc(Br)c2ccc(Cl)nc21 ; OB(O)c1ccccc1C(F)(F)F ; C[Si](C)(C)CCOCn1cc(-c2ccccc2C(F)(F)F)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 2-(trifluoromethyl)phenylboronic acid (189.0 mg, 0.99 mmol), K₂CO₃ (343.8 mg, 2.49 mmol) and Pd(dppf)Cl₂ (60.7 mg, 0.08 mmol) at room temperature under N₂. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (94/6, v/v) to afford 6-chloro-3-[2-(trifluoromethyl)phenyl]-1-[[2-(trimethylsilyl)ethoxy]methyl]pyrrolo[2,3-b]pyridine (300.0 mg, 84%) as a yellow oil. LCMS (ESI, m/z): [M+H]⁺=427.1.

### Step 2: Synthesis of N-[3-[2-(trifluoromethyl)phenyl]-1-[[2-(trimethylsilyl)ethoxy]methyl]pyrrolo[2,3-b]pyridin-6-yl]cyclopropanecarboxamide

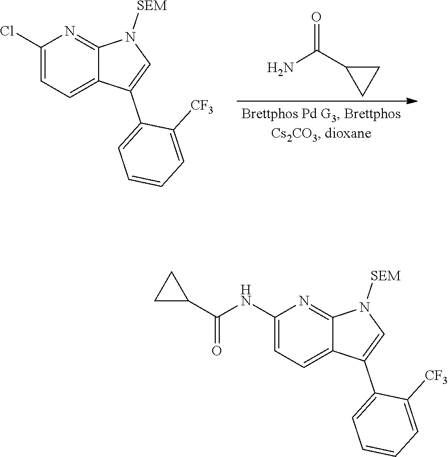 [SMILES: C[Si](C)(C)CCOCn1cc(-c2ccccc2C(F)(F)F)c2ccc(Cl)nc21 ; NC(=O)C1CC1 ; C[Si](C)(C)CCOCn1cc(-c2ccccc2C(F)(F)F)c2ccc(NC(=O)C3CC3)nc21]

To a solution of 6-chloro-3-[2-(trifluoromethyl)phenyl]-1-[[2-(trimethylsilyl)ethoxy]methyl]pyrrolo[2,3-b]pyridine (250.0 mg, 0.59 mmol) in 1,4-dioxane (5.0 mL) was added cyclopropanecarboxamide (149.5 mg, 1.76 mmol), Cs₂CO₃ (572.4 mg, 1.76 mmol), BrettPhos (62.8 mg, 0.12 mmol) and Brettphos Pd G3 (53.1 mg, 0.06 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (84/16, v/v) to afford N-[3-[2-(trifluoromethyl)phenyl]-1-[[2-(trimethylsilyl)ethoxy]methyl]pyrrolo[2,3-b]pyridin-6-yl]cyclopropanecarboxamide (200.0 mg, 71%) as a yellow solid. LCMS (ESI, m/z): [M+H]⁺=476.2.

### Step 3: Synthesis of N-[3-[2-(trifluoromethyl)phenyl]-1H-pyrrolo[2,3-b]pyridin-6-yl]cyclopropanecarboxamide (Compound 7)

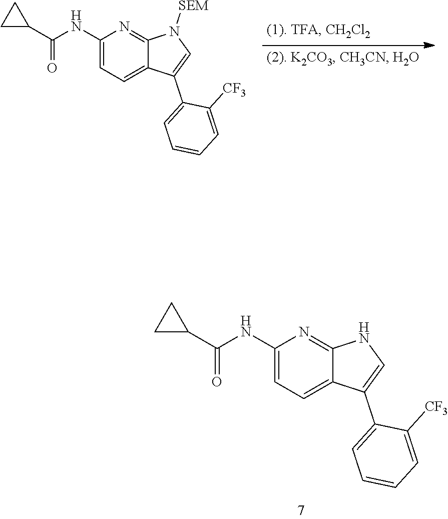 [SMILES: C[Si](C)(C)CCOCn1cc(-c2ccccc2C(F)(F)F)c2ccc(NC(=O)C3CC3)nc21 ; O=C(Nc1ccc2c(-c3ccccc3C(F)(F)F)c[nH]c2n1)C1CC1]

The solution of N-[3-[2-(trifluoromethyl)phenyl]-1-[[2-(trimethylsilyl)ethoxy]methyl]pyrrolo[2,3-b]pyridin-6-yl]cyclopropanecarboxamide (200.0 mg, 0.42 mmol) in CH₂Cl₂/TFA (5.0 mL/5.0 mL) was stirred at room temperature for 1 h. The mixture was evaporated in vacuo. The residue was re-dissolved in ACN/H₂O (5.0/1.0 mL). Then K₂CO₃ (581.2 mg, 4.21 mmol) was added to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 48% B to 61% B in 7 min; 254 nm) to afford N-[3-[2-(trifluoromethyl)phenyl]-1H-pyrrolo[2,3-b]pyridin-6-yl]cyclopropanecarboxamide (21.0 mg, 14%) as a white solid. LCMS (ESI, m/z): [M+H]⁺=346.2. ¹H NMR (300 MHz, DMSO-d₆): δ 11.70 (s, 1H), 10.64 (s, 1H), 7.90-7.84 (m, 2H), 7.73-7.70 (m, 2H), 7.60-7.56 (m, 2H), 7.37 (s, 1H), 2.05-1.95 (m, 1H), 0.85-0.75 (m, 4H).

### Example S8. Compound 8

### Step 1: Synthesis of 6-chloro-3-[2-(trifluoromethoxy)phenyl]-1-[[2-(trimethylsilyl)ethoxy]methyl]pyrrolo[2,3-b]pyridine

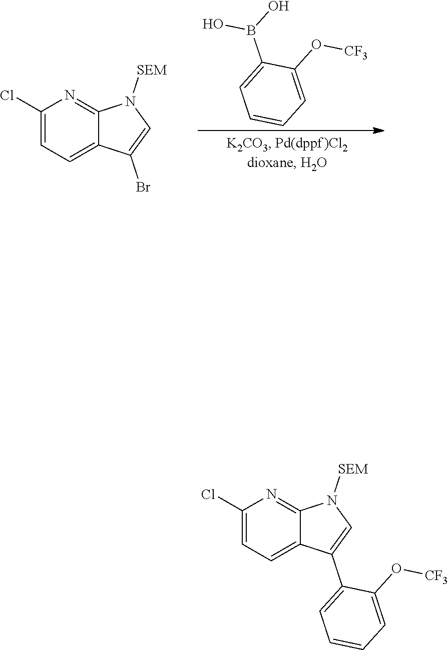 [SMILES: C[Si](C)(C)CCOCn1cc(Br)c2ccc(Cl)nc21 ; OB(O)c1ccccc1OC(F)(F)F ; C[Si](C)(C)CCOCn1cc(-c2ccccc2OC(F)(F)F)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 2-(trifluoromethoxy)phenylboronic acid (204.9 mg, 0.99 mmol), K₂CO₃ (343.8 mg, 2.49 mmol) and Pd(dppf)Cl₂ (60.7 mg, 0.08 mmol) at room temperature under N₂. The resulting mixture was stirred at 80° C. for 3 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-[2-(trifluoromethoxy)phenyl]-1-[[2-(trimethylsilyl)ethoxy]methyl]pyrrolo[2,3-b]pyridine (290.0 mg, 78%) as a yellow oil. LCMS (ESI, m/z): [M+H]⁺=443.1.

### Step 2: Synthesis of N-[3-[2-(trifluoromethoxy)phenyl]-1-[[2-(trimethylsilyl)ethoxy]methyl]pyrrolo[2,3-b]pyridin-6-yl]cyclopropanecarboxamide

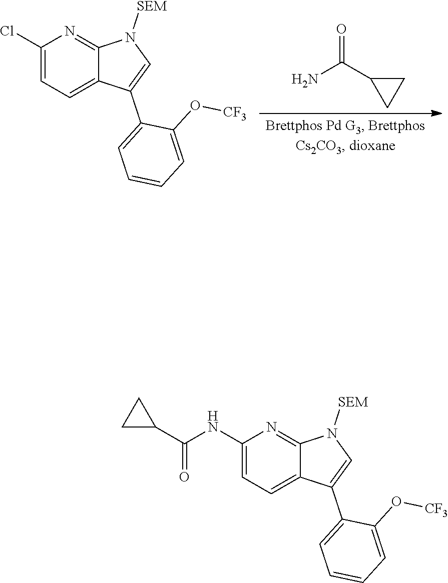 [SMILES: C[Si](C)(C)CCOCn1cc(-c2ccccc2OC(F)(F)F)c2ccc(Cl)nc21 ; NC(=O)C1CC1 ; C[Si](C)(C)CCOCn1cc(-c2ccccc2OC(F)(F)F)c2ccc(NC(=O)C3CC3)nc21]

To a solution of 6-chloro-3-[2-(trifluoromethoxy)phenyl]-1-[[2-(trimethylsilyl)ethoxy]methyl]pyrrolo[2,3-b]pyridine (240.0 mg, 0.54 mmol) in 1,4-dioxane (5.0 mL) was added cyclopropanecarboxamide (138.3 mg, 1.63 mmol), BrettPhos (58.2 mg, 0.11 mmol), Cs₂CO₃ (529.6 mg, 1.63 mmol) and BrettPhos Pd G3 (49.1 mg, 0.05 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford N-[3-[2-(trifluoromethoxy)phenyl]-1-[[2-(trimethylsilyl)ethoxy]methyl]pyrrolo[2,3-b]pyridin-6-yl]cyclopropanecarboxamide (155.0 mg, 58%) as a yellow oil. LCMS (ESI, m/z): [M+H]⁺=492.2.

### Step 3: Synthesis of N-[3-[2-(trifluoromethoxy)phenyl]-1H-pyrrolo[2,3-b]pyridin-6-yl]cyclopropanecarboxamide (Compound 8)

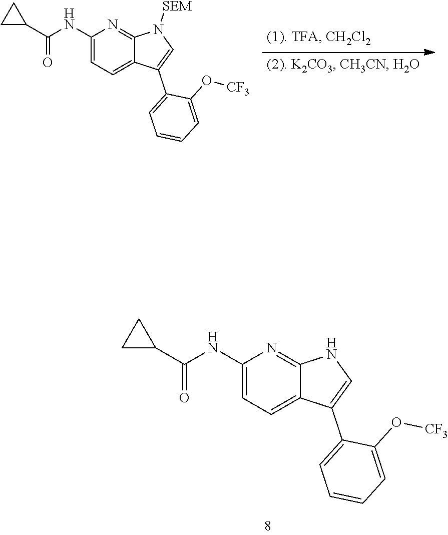 [SMILES: C[Si](C)(C)CCOCn1cc(-c2ccccc2OC(F)(F)F)c2ccc(NC(=O)C3CC3)nc21 ; O=C(Nc1ccc2c(-c3ccccc3OC(F)(F)F)c[nH]c2n1)C1CC1]

To a solution of N-[3-[2-(trifluoromethoxy)phenyl]-1-[[2-(trimethylsilyl)ethoxy]methyl]pyrrolo[2,3-b]pyridin-6-yl]cyclopropanecarboxamide (155.0 mg, 0.32 mmol) in CH₂Cl₂ (5.0 mL) was added TFA (5.0 mL) at room temperature. The resulting mixture was stirred at room temperature for 3 h. After the reaction was completed, the reaction mixture was evaporated in vacuo. The residue was dissolved with ACN/H₂O (5.0/3.0 mL). Then K₂CO₃ (435.8 mg, 3.45 mmol) was added to the mixture. The mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 36% B to 56% B in 11 min; 254 nm) to afford N-[3-[2-(trifluoromethoxy)phenyl]-1H-pyrrolo[2,3-b]pyridin-6-yl]cyclopropanecarboxamide (13.7 mg, 12%) as a white solid. LCMS (ESI, m/z): [M+H]⁺=362.1. ¹H NMR (300 MHz, DMSO-d₆): δ 11.79 (s, 1H), 10.66 (s, 1H), 7.99-7.92 (m, 2H), 7.75-7.71 (m, 1H), 7.60 (d, J=2.4 Hz, 1H), 7.51-7.42 (m, 3H), 2.05-1.95 (m, 1H), 0.83-0.79 (m, 4H).

### Example S9. Compound 9

### Step 1: Synthesis of 2-(6-chloro-1-[[2-(trimethylsilyl)ethoxy]methyl]pyrrolo[2,3-b]pyridin-3-yl)benzonitrile

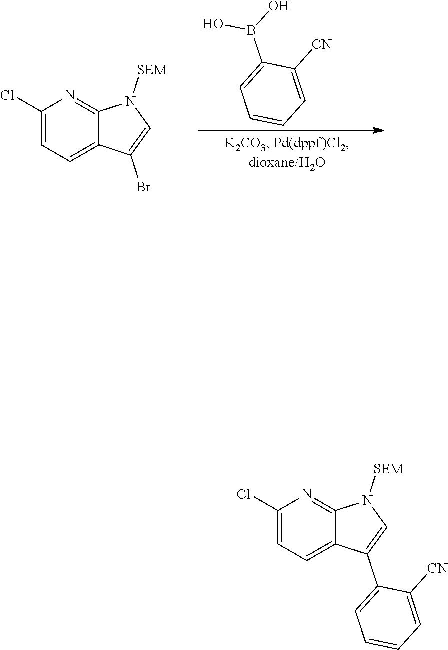 [SMILES: C[Si](C)(C)CCOCn1cc(Br)c2ccc(Cl)nc21 ; N#Cc1ccccc1B(O)O ; C[Si](C)(C)CCOCn1cc(-c2ccccc2C#N)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 2-cyanophenylboronic acid (146.2 mg, 1.00 mmol), K₂CO₃ (343.9 mg, 2.49 mmol) and Pd(dppf)Cl₂ (60.7 mg, 0.08 mmol) at room temperature under N₂. The resulting mixture was stirred at 80° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford 2-(6-chloro-1-[[2-(trimethylsilyl)ethoxy]methyl]pyrrolo[2,3-b]pyridin-3-yl)benzonitrile (190.0 mg, 59%) as a white solid. LCMS (ESI, m/z): [M+H]⁺=384.1.

### Step 2: Synthesis N-[3-(2-cyanophenyl)-1-[[2-(trimethylsilyl)ethoxy]methyl]pyrrolo[2,3-b]pyridin-6-yl]cyclopropanecarboxamide

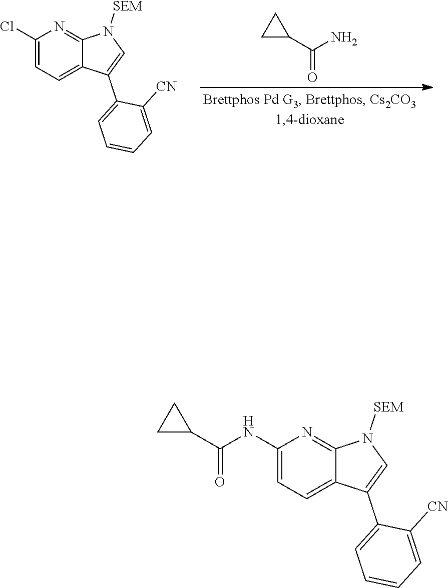 [SMILES: C[Si](C)(C)CCOCn1cc(-c2ccccc2C#N)c2ccc(Cl)nc21 ; NC(=O)C1CC1 ; C[Si](C)(C)CCOCn1cc(-c2ccccc2C#N)c2ccc(NC(=O)C3CC3)nc21]

To a solution of 2-(6-chloro-1-[[2-(trimethylsilyl)ethoxy]methyl]pyrrolo[2,3-b]pyridin-3-yl)benzonitrile (140.0 mg, 0.37 mmol) in 1,4-dioxane (5.0 mL) was added cyclopropanecarboxamide (93.1 mg, 1.09 mmol), Cs₂CO₃ (356.4 mg, 1.09 mmol), Brettphos (39.14 mg, 0.07 mmol) and Brettphos Pd G3 (33.1 mg, 0.04 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford N-[3-(2-cyanophenyl)-1-[[2-(trimethylsilyl)ethoxy]methyl]pyrrolo[2,3-b]pyridin-6-yl]cyclopropanecarboxamide (150.0 mg, 95%) as a yellow solid. LCMS (ESI, m/z): [M+H]⁺=433.2.

### Step 3: Synthesis of N-[3-(2-cyanophenyl)-1H-pyrrolo[2,3-b]pyridin-6-yl]cyclopropanecarboxamide (Compound 9)

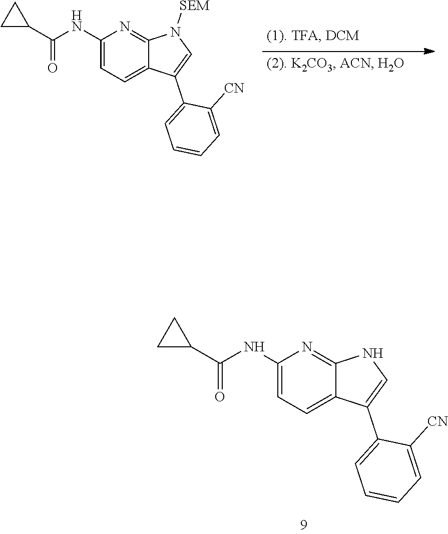 [SMILES: C[Si](C)(C)CCOCn1cc(-c2ccccc2C#N)c2ccc(NC(=O)C3CC3)nc21 ; N#Cc1ccccc1-c1c[nH]c2nc(NC(=O)C3CC3)ccc12]

To a solution of N-[3-(2-cyanophenyl)-1-[[2-(trimethylsilyl)ethoxy]methyl]pyrrolo[2,3-b]pyridin-6-yl]cyclopropanecarboxamide (150.0 mg, 0.35 mmol) in CH₂Cl₂ (5.0 mL) was added TFA (5.0 mL). The resulting mixture stirred at room temperature for 1 h. The reaction mixture was evaporated in vacuo. The residue was re-dissolved in ACN/H₂O (5.0/1.0 mL). Then K₂CO₃ (239.6 mg, 1.73 mmol) was added to the mixture. The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 25% B to 42% B in 11 min; 254 nm) to afford N-[3-(2-cyanophenyl)-1H-pyrrolo[2,3-b]pyridin-6-yl]cyclopropanecarboxamide (24.9 mg, 18%) as a white solid. LCMS (ESI, m/z): [M+H]⁺=303.2. ¹H NMR (300 MHz, DMSO-d₆): δ 11.93 (s, 1H), 10.68 (s, 1H), 8.02-7.91 (m, 3H), 7.79-7.76 (m, 3H), 7.51-7.46 (m, 1H), 2.06-2.03 (m, 1H), 0.83-0.79 (m, 4H).

### Example S10. Compound 10

### Step 1: Synthesis of 3-(6-chloro-1-[[2-(trimethylsilyl)ethoxy]methyl]pyrrolo[2,3-b]pyridin-3-yl)pyridine

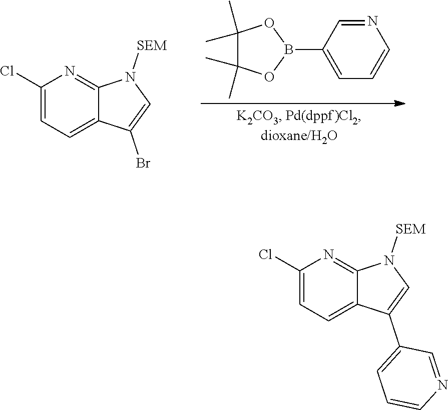 [SMILES: C[Si](C)(C)CCOCn1cc(Br)c2ccc(Cl)nc21 ; CC1(C)OB(c2cccnc2)OC1(C)C ; C[Si](C)(C)CCOCn1cc(-c2cccnc2)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 3-(4,4,5,5-tetramethyl-1,3,2-dioxaborolan-2-yl)pyridine (204.1 mg, 1.0 mmol), K₂CO₃ (343.9 mg, 2.49 mmol) and Pd(dppf)Cl₂ (60.7 mg, 0.08 mmol) at room temperature under N₂. The resulting mixture was stirred at 80° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford 3-(6-chloro-1-[[2-(trimethylsilyl)ethoxy]methyl]pyrrolo[2,3-b]pyridin-3-yl)pyridine (290.0 mg, 97%) as a yellow oil. LCMS (ESI, m/z): [M+H]⁺=360.1.

### Step 2: Synthesis of N-[3-(pyridin-3-yl)-1-[[2-(trimethylsilyl)ethoxy]methyl]pyrrolo[2,3-b]pyridin-6-yl]cyclopropanecarboxamide

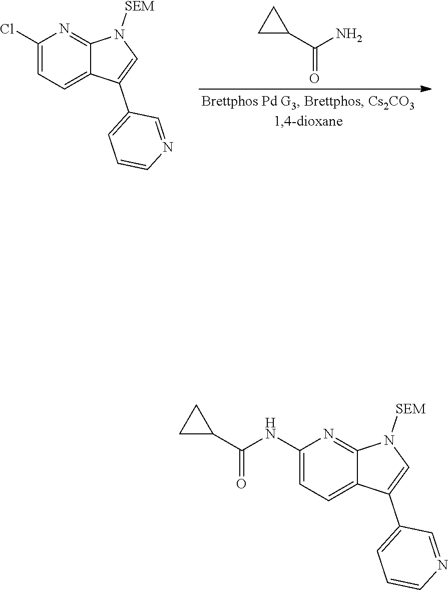 [SMILES: C[Si](C)(C)CCOCn1cc(-c2cccnc2)c2ccc(Cl)nc21 ; NC(=O)C1CC1 ; C[Si](C)(C)CCOCn1cc(-c2cccnc2)c2ccc(NC(=O)C3CC3)nc21]

To a solution of 3-(6-chloro-1-[[2-(trimethylsilyl)ethoxy]methyl]pyrrolo[2,3-b]pyridin-3-yl)pyridine (250.0 mg, 0.69 mmol) in 1,4-dioxane (5.0 mL) was added cyclopropanecarboxamide (177.3 mg, 2.08 mmol), Cs₂CO₃ (678.9 mg, 2.08 mmol), Brettphos (74.6 mg, 0.14 mmol) and Brettphos Pd G3 (62.9 mg, 0.07 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (14/86, v/v) to afford N-[3-(pyridin-3-yl)-1-[[2-(trimethylsilyl)ethoxy]methyl]pyrrolo[2,3-b]pyridin-6-yl]cyclopropanecarboxamide (200.0 mg, 70%) as a yellow solid. LCMS (ESI, m/z): [M+H]⁺=409.2.

### Step 3: Synthesis of N-[3-(pyridin-3-yl)-1H-pyrrolo[2,3-b]pyridin-6-yl]cyclopropanecarboxamide (Compound 10)

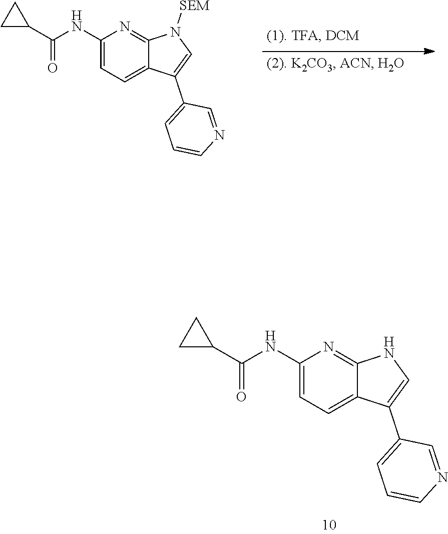 [SMILES: C[Si](C)(C)CCOCn1cc(-c2cccnc2)c2ccc(NC(=O)C3CC3)nc21 ; O=C(Nc1ccc2c(-c3cccnc3)c[nH]c2n1)C1CC1]

To a solution of N-[3-(pyridin-3-yl)-1-[[2-(trimethylsilyl)ethoxy]methyl]pyrrolo[2,3-b]pyridin-6-yl]cyclopropanecarboxamide (150.0 mg, 0.37 mmol) in CH₂Cl₂ (5.0 mL) was added TFA (5.0 mL). The resulting mixture was stirred at room temperature for 1 h. The reaction mixture was evaporated in vacuo. The residue was re-dissolved in ACN/H₂O (5.0/1.0 mL). Then K₂CO₃ (253.7 mg, 1.84 mmol) was added to the mixture. The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH F-Phenyl OBD column, 19×250 mm, 5 um; Mobile Phase A: Water (10 mmol/L NH₄HCO₃), Mobile Phase B: ACN; Flow rate: 25 mL/min; Gradient: 32% B to 51% B in 7 min; 254 nm) to afford N-[3-(pyridin-3-yl)-1H-pyrrolo[2,3-b]pyridin-6-yl]cyclopropanecarboxamide (18.3 mg, 18%) as a white solid. LCMS (ESI, m/z): [M+H]⁺=279.2. ¹H NMR (300 MHz, DMSO-d₆): δ 11.82 (s, 1H), 10.66 (s, 1H), 8.97 (d, J=1.5 Hz, 1H), 8.44 (d, J=3.3 Hz, 1H), 8.30 (d, J=8.7 Hz, 1H), 8.13-8.11 (m, 1H), 7.97 (d, J=9.0 Hz, 1H), 7.91 (d, J=2.4 Hz, 1H), 7.46-7.42 (m, 1H), 2.19-1.90 (m, 1H), 0.84-0.80 (m, 4H).

### Example S11. Compound 11

### Step 1: Synthesis of 4-(6-chloro-1-[[2-(trimethylsilyl)ethoxy]methyl]pyrrolo[2,3-b]pyridin-3-yl)pyridine

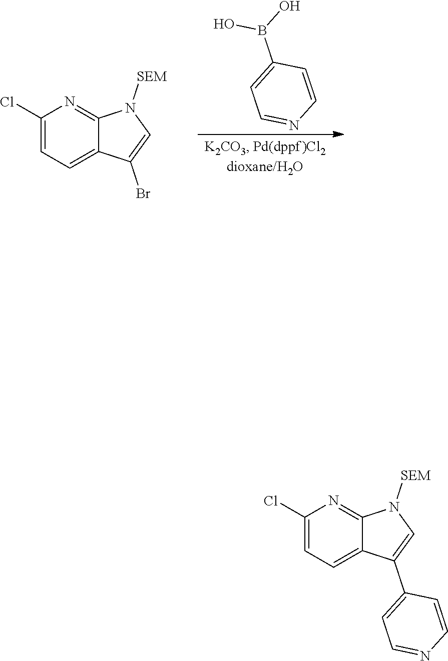 [SMILES: C[Si](C)(C)CCOCn1cc(Br)c2ccc(Cl)nc21 ; OB(O)c1ccncc1 ; C[Si](C)(C)CCOCn1cc(-c2ccncc2)c2ccc(Cl)nc21]

To a solution of 3-bromo-6-chloro-1-[[2-(trimethylsilyl)ethoxy]methyl]pyrrolo[2,3-b]pyridine (280.0 mg, 0.77 mmol) in 1,4-dioxane/H₂O (5.0/1.0 mL) was added pyridin-4-ylboronic acid (95.1 mg, 0.77 mmol), K₂CO₃ (320.9 mg, 2.32 mmol) and Pd(dppf)Cl₂ (113.3 mg, 0.16 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford 4-(6-chloro-1-[[2-(trimethylsilyl)ethoxy]methyl]pyrrolo[2,3-b]pyridin-3-yl)pyridine (114.0 mg, 41%) as a white solid. LCMS (ESI, m/z): [M+H]⁺=360.1.

### Step 2: Synthesis of N-[3-(pyridin-4-yl)-1-[[2-(trimethylsilyl)ethoxy]methyl]pyrrolo[2,3-b]pyridin-6-yl]cyclopropanecarboxamide

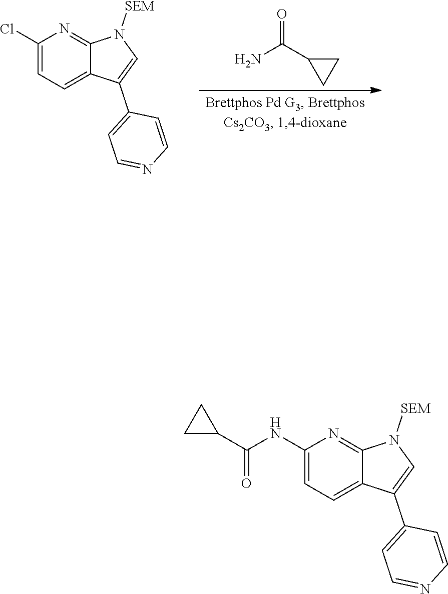 [SMILES: C[Si](C)(C)CCOCn1cc(-c2ccncc2)c2ccc(Cl)nc21 ; NC(=O)C1CC1 ; C[Si](C)(C)CCOCn1cc(-c2ccncc2)c2ccc(NC(=O)C3CC3)nc21]

To a solution of 4-(6-chloro-1-[[2-(trimethylsilyl)ethoxy]methyl]pyrrolo[2,3-b]pyridin-3-yl)pyridine (120.0 mg, 0.33 mmol) in dioxane (8.0 mL) was added cyclopropanecarboxamide (141.9 mg, 1.67 mmol), BrettPhos (71.6 mg, 0.13 mmol), Cs₂CO₃ (325.9 mg, 1.00 mmol) and BrettPhos Pd G3 (60.5 mg, 0.07 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (64/36, v/v) to afford N-[3-(pyridin-4-yl)-1-[[2-(trimethylsilyl)ethoxy]methyl]pyrrolo[2,3-b]pyridin-6-yl]cyclopropanecarboxamide (100.0 mg, 73%) as a white solid. LCMS (ESI, m/z): [M+H]⁺=409.2.

### Step 3: Synthesis of N-[3-(pyridin-4-yl)-1H-pyrrolo[2,3-b]pyridin-6-yl]cyclopropanecarboxamide (Compound 11)

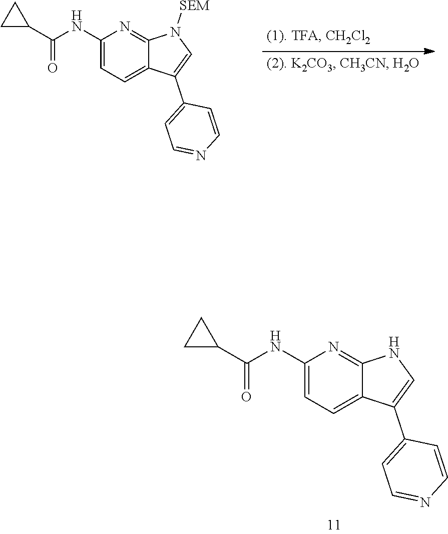 [SMILES: C[Si](C)(C)CCOCn1cc(-c2ccncc2)c2ccc(NC(=O)C3CC3)nc21 ; O=C(Nc1ccc2c(-c3ccncc3)c[nH]c2n1)C1CC1]

To a stirred mixture of N-[3-(pyridin-4-yl)-1-[[2-(trimethylsilyl)ethoxy]methyl]pyrrolo[2,3-b]pyridin-6-yl]cyclopropanecarboxamide (100.0 mg, 0.25 mmol) in DCM (3.0 mL) was added TFA (3.0 mL). The resulting mixture was stirred at room temperature for 3 h. The mixture was evaporated in vacuo. The residue was re-dissolved in ACN/H₂O (5.0/1.0 mL). Then K₂CO₃ (182.7 mg, 1.32 mmol) was added to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH F-Phenyl OBD column, 19×250 mm, 5 um; Mobile Phase A: Water (10 mmol/L NH₄HCO₃), Mobile Phase B: ACN; Flow rate: 25 mL/min; Gradient: 30% B to 60% B in 7 min; 254 nm) to afford N-[3-(pyridin-4-yl)-1H-pyrrolo[2,3-b]pyridin-6-yl]cyclopropanecarboxamide (12.8 mg, 10%) as a white solid. LCMS (ESI, m/z): [M+H]⁺=279.1. ¹H NMR (400 MHz, DMSO-d₆): δ 11.95 (s, 1H), 10.65 (s, 1H), 8.53 (d, J=4.8 Hz, 2H), 8.39 (d, J=8.8 Hz, 1H), 8.08 (s, 1H), 7.99 (d, J=8.8 Hz, 1H), 7.74 (d, J=4.8 Hz, 2H), 2.05-1.97 (m, 1H), 0.83-0.79 (m, 4H).

### Example S12. Compound 12

### Step 1: Synthesis of 2-(6-chloro-1-[[2-(trimethylsilyl)ethoxy]methyl]pyrrolo[2,3-b]pyridin-3-yl)phenol

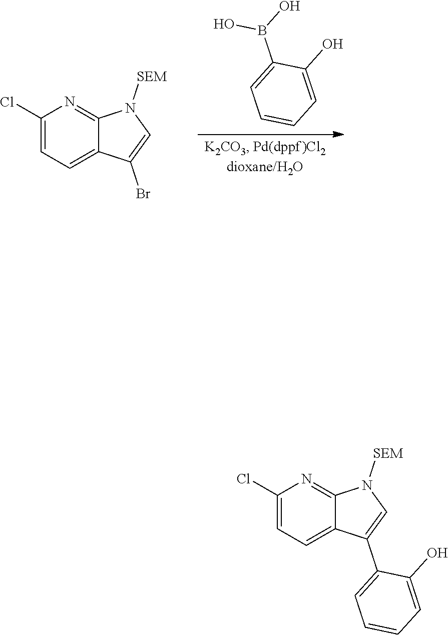 [SMILES: C[Si](C)(C)CCOCn1cc(Br)c2ccc(Cl)nc21 ; OB(O)c1ccccc1O ; C[Si](C)(C)CCOCn1cc(-c2ccccc2O)c2ccc(Cl)nc21]

To a solution of 3-bromo-6-chloro-1-[[2-(trimethylsilyl)ethoxy]methyl]pyrrolo[2,3-b]pyridine (280.0 mg, 0.77 mmol) in 1,4-dioxane/H₂O (5.0/1.0 mL) was added 2-hydroxyphenylboronic acid (106.8 mg, 0.77 mmol), K₂CO₃ (320.9 mg, 2.32 mmol) and Pd(dppf)Cl₂ (113.3 mg, 0.16 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (89/11, v/v) to afford 2-(6-chloro-1-[[2-(trimethylsilyl)ethoxy]methyl]pyrrolo[2,3-b]pyridin-3-yl)phenol (110.0 mg, 38%) as a white solid. LCMS (ESI, m/z): [M+H]⁺=375.1.

### Step 2: Synthesis of N-[3-(2-hydroxyphenyl)-1-[[2-(trimethylsilyl)ethoxy]methyl]pyrrolo[2,3-b]pyridin-6-yl]cyclopropanecarboxamide

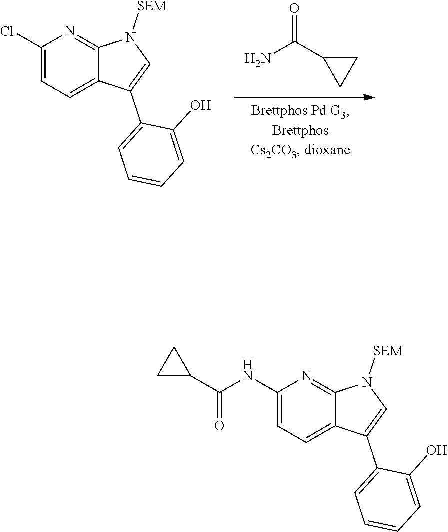 [SMILES: C[Si](C)(C)CCOCn1cc(-c2ccccc2O)c2ccc(Cl)nc21 ; NC(=O)C1CC1 ; C[Si](C)(C)CCOCn1cc(-c2ccccc2O)c2ccc(NC(=O)C3CC3)nc21]

To a solution of 2-(6-chloro-1-[[2-(trimethylsilyl)ethoxy]methyl]pyrrolo[2,3-b]pyridin-3-yl)phenol (110.0 mg, 0.29 mmol) in dioxane (8.0 mL) was added cyclopropanecarboxamide (124.8 mg, 1.47 mmol), BrettPhos (63.0 mg, 0.12 mmol), Cs₂CO₃ (286.8 mg, 0.88 mmol) and BrettPhos Pd G3 (53.2 mg, 0.06 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (63/37, v/v) to afford N-[3-(2-hydroxyphenyl)-1-[[2-(trimethylsilyl)ethoxy]methyl]pyrrolo[2,3-b]pyridin-6-yl]cyclopropanecarboxamide (52.0 mg, 42%) as a white solid. LCMS (ESI, m/z): [M+H]⁺=424.2.

### Step 3: Synthesis of N-[3-(2-hydroxyphenyl)-1H-pyrrolo[2,3-b]pyridin-6-yl]cyclopropanecarboxamide (Compound 12)

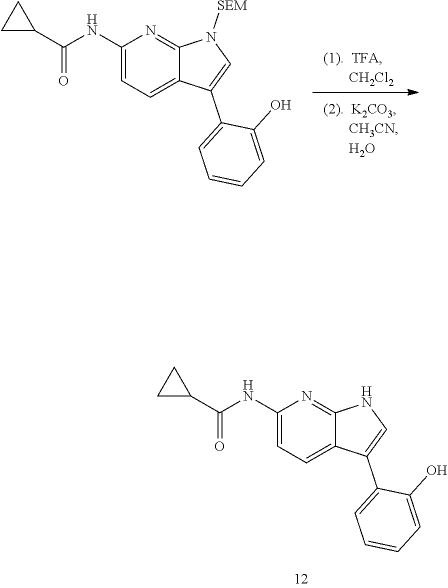 [SMILES: C[Si](C)(C)CCOCn1cc(-c2ccccc2O)c2ccc(NC(=O)C3CC3)nc21 ; O=C(Nc1ccc2c(-c3ccccc3O)c[nH]c2n1)C1CC1]

To a stirred mixture of N-[3-(2-hydroxyphenyl)-1-[[2-(trimethylsilyl)ethoxy]methyl]pyrrolo[2,3-b]pyridin-6-yl]cyclopropanecarboxamide (110.0 mg, 0.26 mmol) in DCM (3.0 mL) was added TFA (3.0 mL). The resulting mixture was stirred at room temperature for 3 h. The mixture was evaporated in vacuo. The residue was re-dissolved in ACN/H₂O (5.0/1.0 mL). Then K₂CO₃ (107.7 mg, 0.78 mmol) was added to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 24% B to 35% B in 12 min; 254 nm) to afford N-[3-(2-hydroxyphenyl)-1H-pyrrolo[2,3-b]pyridin-6-yl]cyclopropanecarboxamide (16.9 mg, 20%) as a white solid. LCMS (ESI, m/z): [M+H]⁺=294.2. ¹H NMR (400 MHz, DMSO-d₆): δ 11.47 (s, 1H), 10.55 (s, 1H), 9.48 (s, 1H), 8.05 (d, J=8.4 Hz, 1H), 7.87 (d, J=8.4 Hz, 1H), 7.61 (s, 1H), 7.50 (d, J=8.0 Hz, 1H), 7.08-7.05 (m, 1H), 6.94 (d, J=7.6 Hz, 1H), 6.89-6.86 (m, 1H), 2.05-1.97 (m, 1H), 0.82-0.78 (m, 4H).

### Example S13. Compound 13

### Step 1: Synthesis of 3-(6-chloro-1-[[2-(trimethylsilyl)ethoxy]methyl]pyrrolo[2,3-b]pyridin-3-yl)phenol

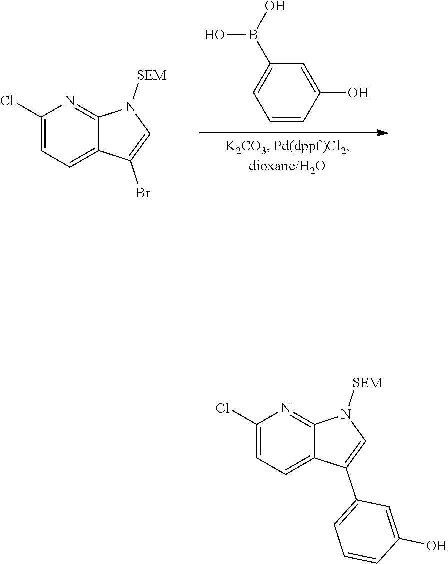 [SMILES: C[Si](C)(C)CCOCn1cc(Br)c2ccc(Cl)nc21 ; OB(O)c1cccc(O)c1 ; C[Si](C)(C)CCOCn1cc(-c2cccc(O)c2)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (5.0/1.0 mL) was added 3-hydroxyphenylboronic acid (137.3 mg, 0.99 mmol), K₂CO₃ (343.9 mg, 2.49 mmol) and Pd(dppf)Cl₂ (60.7 mg, 0.08 mmol) at room temperature under N₂. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 3-(6-chloro-1-[[2-(trimethylsilyl)ethoxy]methyl]pyrrolo[2,3-b]pyridin-3-yl)phenol (230.0 mg, 73%) as a yellow oil. LCMS (ESI, m/z): [M+H]⁺=375.1.

### Step 2: Synthesis of N-[3-(3-hydroxyphenyl)-1-[[2-(trimethylsilyl)ethoxy]methyl]pyrrolo[2,3-b]pyridin-6-yl]cyclopropanecarboxamide

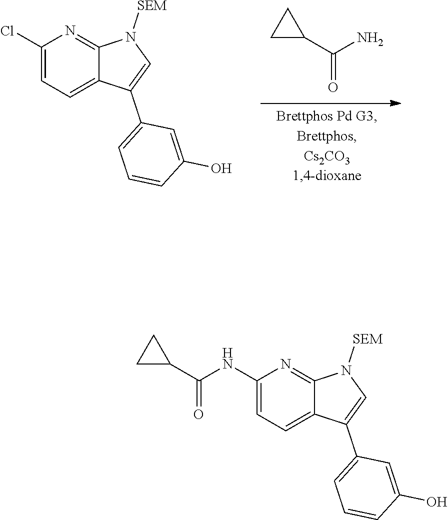 [SMILES: C[Si](C)(C)CCOCn1cc(-c2cccc(O)c2)c2ccc(Cl)nc21 ; NC(=O)C1CC1 ; C[Si](C)(C)CCOCn1cc(-c2cccc(O)c2)c2ccc(NC(=O)C3CC3)nc21]

To a solution of 3-(6-chloro-1-[[2-(trimethylsilyl)ethoxy]methyl]pyrrolo[2,3-b]pyridin-3-yl)phenol (200.0 mg, 0.53 mmol) in 1,4-dioxane (5.0 mL) was added cyclopropanecarboxamide (136.2 mg, 1.60 mmol), Cs₂CO₃ (521.4 mg, 1.60 mmol), Brettphos (57.3 mg, 0.11 mmol) and Brettphos Pd G3 (48.4 mg, 0.05 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford N-[3-(3-hydroxyphenyl)-1-[[2-(trimethylsilyl)ethoxy]methyl]pyrrolo[2,3-b]pyridin-6-yl]cyclopropanecarboxamide (220.0 mg, 97%) as a yellow solid. LCMS (ESI, m/z): [M+H]⁺=424.2.

### Step 3: Synthesis of N-[3-(3-hydroxyphenyl)-1H-pyrrolo[2,3-b]pyridin-6-yl]cyclopropanecarboxamide (Compound 13)

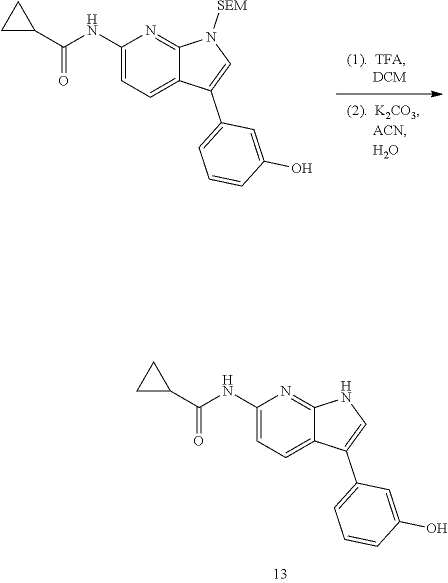 [SMILES: C[Si](C)(C)CCOCn1cc(-c2cccc(O)c2)c2ccc(NC(=O)C3CC3)nc21 ; O=C(Nc1ccc2c(-c3cccc(O)c3)c[nH]c2n1)C1CC1]

The solution of N-[3-(3-hydroxyphenyl)-1-[[2-(trimethylsilyl)ethoxy]methyl]pyrrolo[2,3-b]pyridin-6-yl]cyclopropanecarboxamide (170.0 mg, 0.40 mmol) in CH₂Cl₂/TFA (5.0 mL/5.0 mL) was stirred at room temperature for 1 h. The mixture was evaporated in vacuo. The residue was re-dissolved in ACN/H₂O (5.0/1.0 mL). Then K₂CO₃ (554.7 mg, 4.01 mmol) was added to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 29% B to 32% B in 8 min; 254 nm) to afford N-[3-(3-hydroxyphenyl)-1H-pyrrolo[2,3-b]pyridin-6-yl]cyclopropanecarboxamide (34.0 mg, 28%) as a white solid. LCMS (ESI, m/z): [M+H]⁺=294.2. ¹H NMR (300 MHz, DMSO-d₆): δ 11.62 (s, 1H), 10.62 (s, 1H), 9.38 (s, 1H), 8.18 (d, J=8.7 Hz, 1H), 7.94 (d, J=8.7 Hz, 1H), 7.66 (d, J=2.7 Hz, 1H), 7.24-7.19 (m, 1H), 7.14-7.11 (m, 2H), 6.67-6.63 (m, 1H), 2.08-2.03 (m, 1H), 0.83-0.79 (m, 4H).

### Example S14. Compound 14

### Step 1: Synthesis of 3-(6-chloro-1-[[2-(trimethylsilyl)ethoxy]methyl]pyrrolo[2,3-b]pyridin-3-yl)-2-methoxypyridine

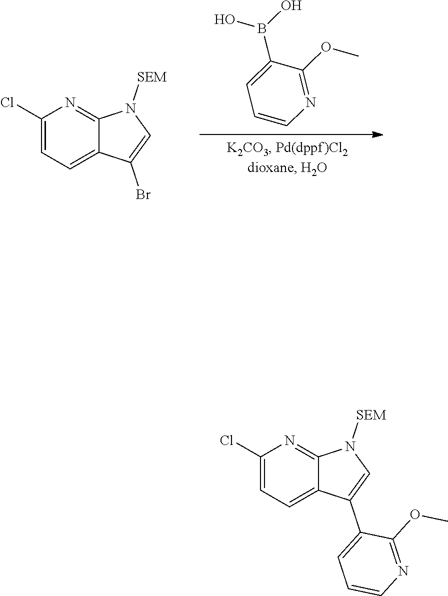 [SMILES: C[Si](C)(C)CCOCn1cc(Br)c2ccc(Cl)nc21 ; COc1ncccc1B(O)O ; COc1ncccc1-c1cn(COCC[Si](C)(C)C)c2nc(Cl)ccc12]

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (10.0/2.0 mL) was added 2-methoxypyridin-3-ylboronic acid (211.4 mg, 1.38 mmol), K₂CO₃ (57.3 mg, 0.42 mmol) and Pd(dppf)Cl₂ (112.9 mg, 0.14 mmol). The resulting mixture was stirred at 80° C. for 2 h under N₂. After the reaction was completed, the resulting mixture was concentrated under vacuum. The residue was purified by flash column chromatography with petroleum ether/EtOAc (80/20, v/v) to afford 3-(6-chloro-1-[[2-(trimethylsilyl)ethoxy]methyl]pyrrolo[2,3-b]pyridin-3-yl)-2-methoxypyridine (250.0 mg, 46%) as a yellow solid. LCMS (ESI, m/z): [M+H]⁺=390.1.

### Step 2: Synthesis of N-(3-(2-methoxypyridin-3-yl)-1-((2-(trimethylsilyl)ethoxy)methyl)-1H-pyrrolo[2,3-b]pyridin-6-yl)cyclopropanecarboxamide

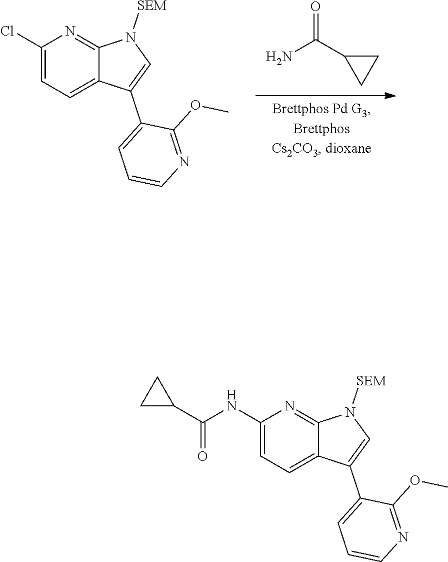 [SMILES: COc1ncccc1-c1cn(COCC[Si](C)(C)C)c2nc(Cl)ccc12 ; NC(=O)C1CC1 ; COc1ncccc1-c1cn(COCC[Si](C)(C)C)c2nc(NC(=O)C3CC3)ccc12]

To a solution of 3-(6-chloro-1-[[2-(trimethylsilyl)ethoxy]methyl]pyrrolo[2,3-b]pyridin-3-yl)-2-methoxypyridine (235.0 mg, 0.60 mmol) in 1,4-dioxane (8.0 mL) was added cyclopropanecarboxamide (205.2 mg, 2.41 mmol), BrettPhos (64.7 mg, 0.12 mmol), Cs₂CO₃ (589.0 mg, 1.81 mmol) and Brettphos Pd G3 (54.6 mg, 0.06 mmol). The resulting mixture was stirred at 100° C. for 2 h under N₂. After the reaction was completed, the resulting mixture was concentrated under vacuum. The residue was purified by flash column chromatography with ether/ethyl acetate (75/25, v/v) to afford N-(3-(2-methoxypyridin-3-yl)-1-((2-(trimethylsilyl)ethoxy)methyl)-1H-pyrrolo[2,3-b]pyridin-6-yl)cyclopropanecarboxamide (250.0 mg, 94%) as a yellow solid. LCMS (ESI, m/z): [M+H]⁺=439.2.

### Step 3: Synthesis of N-[3-(2-methoxypyridin-3-yl)-1H-pyrrolo[2,3-b]pyridin-6-yl]cyclopropanecarboxamide (Compound 14)

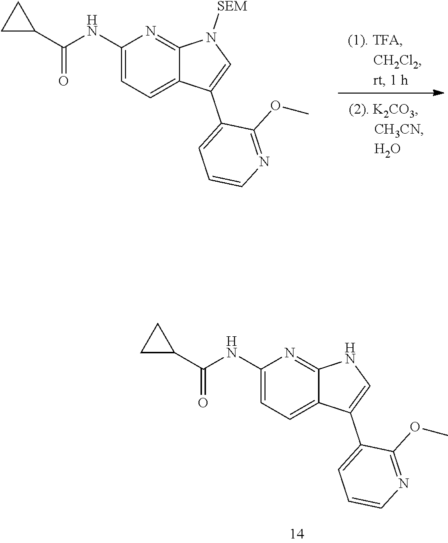 [SMILES: COc1ncccc1-c1cn(COCC[Si](C)(C)C)c2nc(NC(=O)C3CC3)ccc12 ; COc1ncccc1-c1c[nH]c2nc(NC(=O)C3CC3)ccc12]

To a solution of N-[3-(2-methoxypyridin-3-yl)-1-[[2-(trimethylsilyl)ethoxy]methyl]pyrrolo[2,3-b]pyridin-6-yl]cyclopropanecarboxamide (250.0 mg, 0.57 mmol) in CH₂Cl₂ (2.0 mL) was added TFA (2.0 mL). The resulting mixture was stirred at room temperature for 1 h. The mixture was concentrated under vacuum. The residue was re-dissolved in ACN (6.0 mL) and H₂O (0.5 mL). Then K₂CO₃ (393.9 mg, 2.85 mmol) was added to the mixture. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ether/ethyl acetate (82/18, v/v) and then purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate: 60 mL/min; Gradient: 26% B to 56% B in 10 min; 254 nm) to afford N-[3-(2-methoxypyridin-3-yl)-1H-pyrrolo[2,3-b]pyridin-6-yl]cyclopropanecarboxamide (51.0 mg, 29%) as a white solid. LCMS (ESI, m/z): [M+H]⁺=309.2. ¹H NMR (300 MHz, DMSO-d₆): δ 11.68 (s, 1H), 10.59 (s, 1H), 8.11-8.07 (m, 2H), 8.01-7.91 (m, 2H), 7.72 (d, J=2.7 Hz, 1H), 7.10-7.06 (m, 1H), 3.95 (s, 3H), 2.08-2.01 (m, 1H), 0.87-0.79 (m, 4H).

### Example S15. Compound 15

### Step 1: Synthesis of 5-bromo-4-methoxy-2,1,3-benzoxadiazol-1-ium-1-olate

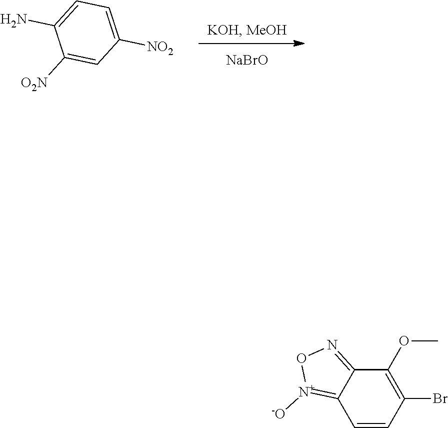

To a mixture of 2,4-dinitroaniline (10.0 g, 54.61 mmol) and KOH (9.2 g, 163.80 mmol) in MeOH (1.0 L) was added dropwise a solution of potassium hydroxide (64.9 g, 546.12 mmol) in H₂O (1.0 L) 50° C. under N₂. The resulting mixture was stirred at 50° C. for 3 h. After the reaction was completed, the reaction was cooled to room temperature and then filtered. The precipitated solid was collected and dried to afford 5-bromo-4-methoxy-2,1,3-benzoxadiazol-1-ium-1-olate (2.1 g, 16%) as a yellow solid. LCMS (ESI, m/z): [M+H]⁺=244.9.

### Step 2: Synthesis of 4-bromo-3-methoxybenzene-1,2-diamine

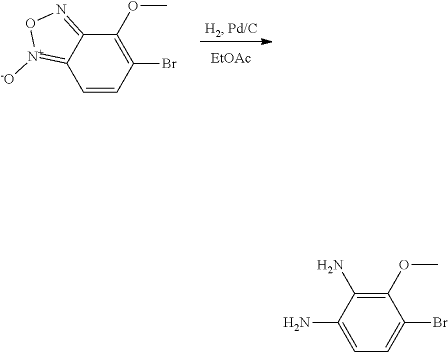

To a solution of 5-bromo-4-methoxy-2,1,3-benzoxadiazol-1-ium-1-olate (1.2 g, 4.90 mmol) in EtOAc (30.0 mL) was added Pd/C (0.1 g, dry). The resulting mixture was stirred at room temperature for 16 h under H2. After the reaction was completed, the resulting mixture was filtered. The filtrate was concentrated under reduced pressure to afford 4-bromo-3-methoxybenzene-1,2-diamine (623.0 mg, crude) as a brown solid. LCMS (ESI, m/z): [M+H]⁺=217.0.

### Step 3: Synthesis of 5-bromo-4-methoxy-3H-1,3-benzodiazole

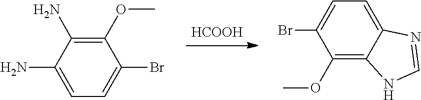

The solution of 4-bromo-3-methoxybenzene-1,2-diamine (623.0 mg, 2.87 mmol) in HCOOH (10.0 mL) was stirred at 100° C. for 2 h. After the reaction was completed, the reaction was diluted with H₂O. The pH value of the mixture was adjusted to 8 with NaOH (aq.) and then extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 5-bromo-4-methoxy-3H-1,3-benzodiazole (495.0 mg, 75%) as a red solid. LCMS (ESI, m/z): [M+H]⁺=227.0.

### Step 4: Synthesis of 6-bromo-7-methoxy-1-[[2-(trimethylsilyl)ethoxy]methyl]-1,3-benzodiazole

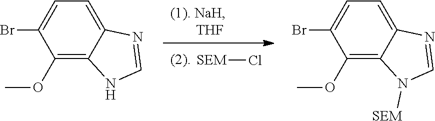 [SMILES: COc1c(Br)ccc2nc[nH]c12 ; C[Si](C)(C)CCOCCl ; COc1c(Br)ccc2ncn(COCC[Si](C)(C)C)c12]

To a solution of 5-bromo-4-methoxy-3H-1,3-benzodiazole (710.0 mg, 3.13 mmol) in THE (20.0 mL) was added NaH (375.2 mg, 60%) at 0° C. under N₂. The resulting mixture was stirred at 0° C. for 1 h. Then SEM-Cl (782.0 mg, 4.69 mmol) was added dropwise to the mixture at 0° C. under N₂. The resulting mixture was stirred at 0° C. for 1 h under N₂. After the reaction was completed, the reaction was quenched with H₂O and extracted with ethyl acetate. The combined organic layer was washed with brine, dried over anhydrous sodium sulfate and filtered. The filtrate was concentrated under reduced pressure to afford 6-bromo-7-methoxy-1-[[2-(trimethylsilyl)ethoxy]methyl]-1,3-benzodiazole (790.0 mg, crude) as a black oil. LCMS (ESI, m/z): [M+H]⁺=357.1.

### Step 5: Synthesis of 7-methoxy-6-(4,4,5,5-tetramethyl-1,3,2-dioxaborolan-2-yl)-1-[[2-(trimethylsilyl)ethoxy]methyl]-1,3-benzodiazole

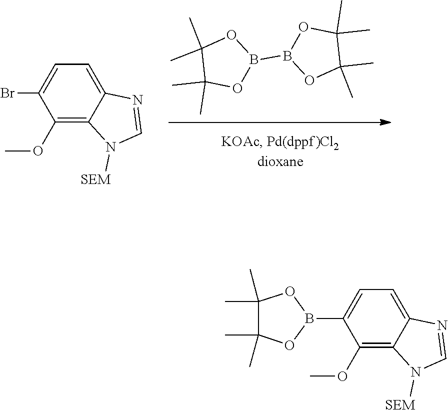

To a solution of 6-bromo-7-methoxy-1-[[2-(trimethylsilyl)ethoxy]methyl]-1,3-benzodiazole (740.0 mg, 2.07 mmol) in dioxane (20.0 mL) was added bis(pinacolato)diboron (1577.7 mg, 6.21 mmol), KOAc (609.7 mg, 6.21 mmol) and Pd(dppf)Cl₂ (169.1 mg, 0.21 mmol) at room temperature under N₂. The resulting mixture was stirred at 85° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (6/1, v/v) to afford 7-methoxy-6-(4,4,5,5-tetramethyl-1,3,2-dioxaborolan-2-yl)-1-[[2-(trimethylsilyl)ethoxy]methyl]-1,3-benzodiazole (270.0 mg, 32%) as a light yellow oil. LCMS (ESI, m/z): [M+H]⁺=405.2.

### Step 6: Synthesis of 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl]-1,3-benzodiazole

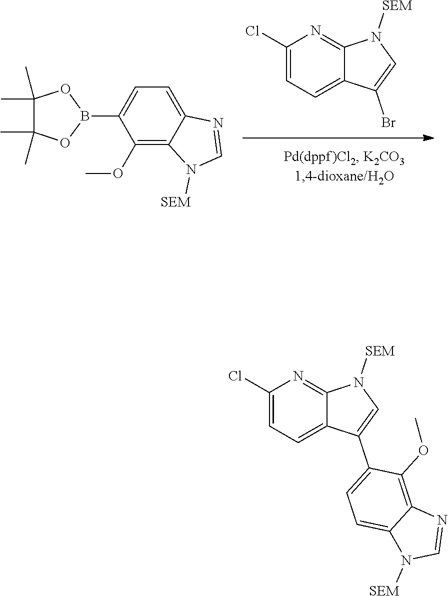 [SMILES: COc1c(B2OC(C)(C)C(C)(C)O2)ccc2ncn(COCC[Si](C)(C)C)c12 ; C[Si](C)(C)CCOCn1cc(Br)c2ccc(Cl)nc21 ; COc1c(-c2cn(COCC[Si](C)(C)C)c3nc(Cl)ccc23)ccc2c1ncn2COCC[Si](C)(C)C]

To a solution of 7-methoxy-6-(4,4,5,5-tetramethyl-1,3,2-dioxaborolan-2-yl)-1-[[2-(trimethylsilyl)ethoxy]methyl]-1,3-benzodiazole (405.0 mg, 1.00 mmol) in dioxane/H₂O (10.0 mL/2.0 mL) was added 3-bromo-6-chloro-1-[[2-(trimethylsilyl)ethoxy]methyl]pyrrolo[2,3-b]pyridine (301.9 mg, 0.84 mmol), K₂CO₃ (346.0 mg, 2.50 mmol) and Pd(dppf)Cl₂ (68.2 mg, 0.08 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl]-1,3-benzodiazole (140.0 mg, 30%) as a yellow solid. LCMS (ESI, m/z): [M+H]⁺=559.2.

### Step 7: Synthesis of N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropanecarboxamide

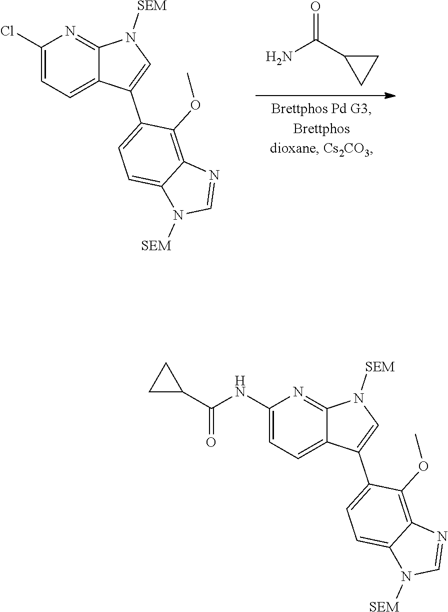 [SMILES: COc1c(-c2cn(COCC[Si](C)(C)C)c3nc(Cl)ccc23)ccc2c1ncn2COCC[Si](C)(C)C ; NC(=O)C1CC1 ; COc1c(-c2cn(COCC[Si](C)(C)C)c3nc(NC(=O)C4CC4)ccc23)ccc2c1ncn2COCC[Si](C)(C)C]

To a solution of 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl]-1,3-benzodiazole (122.0 mg, 0.22 mmol) in 1,4-dioxane (6.0 mL) was added cyclopropanecarboxamide (92.8 mg, 1.09 mmol), BrettPhos (23.4 mg, 0.04 mmol), Cs₂CO₃ (213.2 mg, 0.65 mmol) and BrettPhos Pd G3 (19.7 mg, 0.02 mmol) at room temperature under N₂. The resulting mixture was irradiated with microwave radiation at 120° C. for 1.5 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropanecarboxamide (109.0 mg, 82%) as a yellow oil. LCMS (ESI, m/z): [M+H]⁺=608.3.

### Step 8: Synthesis of N-[3-(4-methoxy-1H-1,3-benzodiazol-5-yl)-1H-pyrrolo[2,3-b]pyridin-6-yl]cyclopropanecarboxamide (Compound 15)

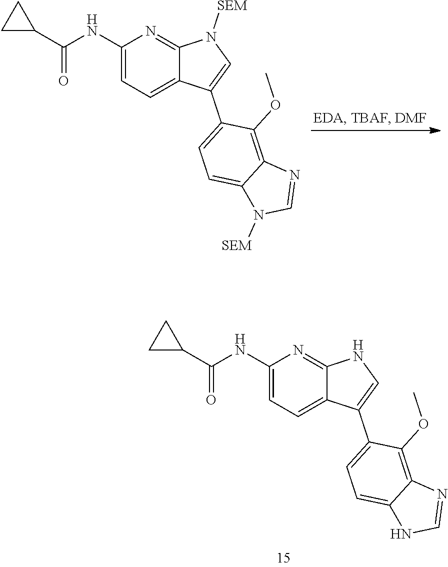 [SMILES: COc1c(-c2cn(COCC[Si](C)(C)C)c3nc(NC(=O)C4CC4)ccc23)ccc2c1ncn2COCC[Si](C)(C)C ; COc1c(-c2c[nH]c3nc(NC(=O)C4CC4)ccc23)ccc2[nH]cnc12]

To a solution of N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropanecarboxamide (109.0 mg, 0.18 mmol) in DMF (5.0 mL) was added ethane-1,2-diamine (53.8 mg, 0.90 mmol) and TBAF (0.5 mL, 0.01 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 16% B to 38% B in 6 min; 254 nm) to afford N-[3-(4-methoxy-1H-1,3-benzodiazol-5-yl)-1H-pyrrolo[2,3-b]pyridin-6-yl]cyclopropanecarboxamide (9.1 mg, 15%) as a white solid. LCMS (ESI, m/z): [M+H]⁺=348.2. ¹H NMR (300 MHz, DMSO-d₆): δ 12.49 (s, 1H), 11.47 (s, 1H), 10.58 (s, 1H), 8.21-8.18 (m, 1H), 7.97-7.87 (m, 2H), 7.47 (s, 1H), 7.36-7.33 (m, 1H), 7.26-7.24 (m, 1H), 4.22 (s, 2H), 3.66 (s, 1H), 2.10-2.01 (m, 1H), 0.83-0.79 (m, 4H).

### Example S16. Compound 16

### Step 1: Synthesis of 5-bromo-4-methoxy-1-[[2-(trimethylsilyl)ethoxy]methyl]indazole

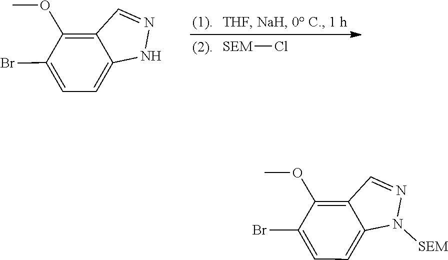 [SMILES: COc1c(Br)ccc2[nH]ncc12 ; C[Si](C)(C)CCOCCl ; COc1c(Br)ccc2c1cnn2COCC[Si](C)(C)C]

To a mixture of 5-bromo-4-methoxy-1H-indazole (1.0 g, 4.40 mmol) in THF (10.0 mL) was added NaH (0.3 g, 60%) at 0° C. under N₂. The resulting mixture was stirred at 0° C. for 1 h. Then SEM-Cl (1.1 g, 6.62 mmol) was added dropwise to the mixture at 0° C. The resulting mixture was stirred at room temperature for another 2 h. After the reaction was completed, the reaction mixture was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 5-bromo-4-methoxy-1-[[2-(trimethylsilyl)ethoxy]methyl]indazole (2.5 g, crude) as a yellow oil. LCMS (ESI, m/z): [M+H]⁺=357.1.

### Step 2: Synthesis of 4-methoxy-5-(4,4,5,5-tetramethyl-1,3,2-dioxaborolan-2-yl)-1-[[2-(trimethylsilyl)ethoxy] methyl]indazole

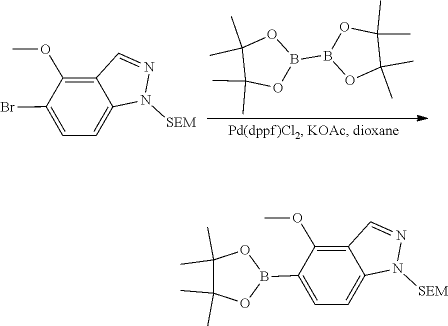

To a mixture of 5-bromo-4-methoxy-1-[[2-(trimethylsilyl)ethoxy]methyl]indazole (2.4 g, 6.71 mmol) and 4,4,4′,4′,5,5,5′,5′-octamethyl-2,2′-bi(1,3,2-dioxaborolane) (5.1 g, 20.16 mmol) in dioxane (30.0 mL) was added Pd(dppf)Cl₂ (0.5 g, 0.67 mmol) and KOAc (1.9 g, 20.17 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 4-methoxy-5-(4,4,5,5-tetramethyl-1,3,2-dioxaborolan-2-yl)-1-[[2-(trimethylsilyl)ethoxy] methyl]indazole (750.0 mg, 27%) as a yellow oil. LCMS (ESI, m/z): [M+H]⁺=405.2.

### Step 3: Synthesis of 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl] indazole

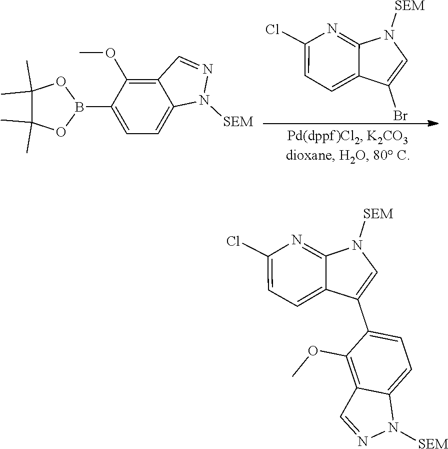 [SMILES: COc1c(B2OC(C)(C)C(C)(C)O2)ccc2c1cnn2COCC[Si](C)(C)C ; C[Si](C)(C)CCOCn1cc(Br)c2ccc(Cl)nc21 ; COc1c(-c2cn(COCC[Si](C)(C)C)c3nc(Cl)ccc23)ccc2c1cnn2COCC[Si](C)(C)C]

To a mixture of 4-methoxy-5-(4,4,5,5-tetramethyl-1,3,2-dioxaborolan-2-yl)-1-[[2-(trimethylsilyl)ethoxy]methyl]indazole (660.0 mg, 1.63 mmol) and 3-bromo-6-chloro-1-[[2-(trimethylsilyl)ethoxy]methyl]pyrrolo[2,3-b]pyridine (400.0 mg, 1.11 mmol) in dioxane/H₂O (10.0/1.0 mL) was added Pd(dppf)Cl₂ (90.0 mg, 0.11 mmol) and K₂CO₃ (460.0 mg, 3.33 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl] indazole (330.0 mg, 36%) as a yellow solid. LCMS (ESI, m/z): [M+H]⁺=559.2.

### Step 4: Synthesis of N-[3-(4-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropanecarboxamide

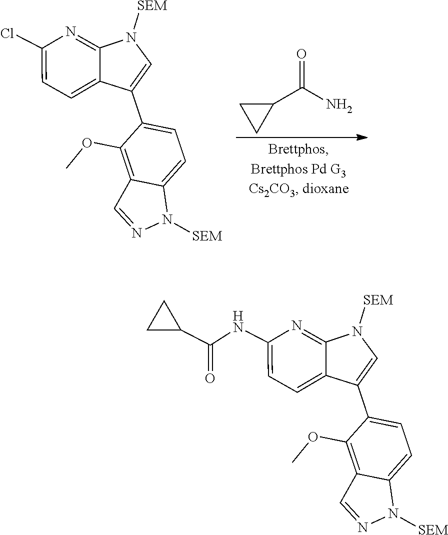 [SMILES: COc1c(-c2cn(COCC[Si](C)(C)C)c3nc(Cl)ccc23)ccc2c1cnn2COCC[Si](C)(C)C ; NC(=O)C1CC1 ; COc1c(-c2cn(COCC[Si](C)(C)C)c3nc(NC(=O)C4CC4)ccc23)ccc2c1cnn2COCC[Si](C)(C)C]

To a mixture of 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl]indazole (300.0 mg, 0.53 mmol) and cyclopropanecarboxamide (182.6 mg, 2.14 mmol) in dioxane (5.0 mL) was added BrettPhos Pd G3 (48.6 mg, 0.05 mmol), BrettPhos (57.5 mg, 0.10 mmol) and Cs₂CO₃ (699.1 mg, 2.14 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford N-[3-(4-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropanecarboxamide (220.0 mg, 67%) as a yellow solid. LCMS (ESI, m/z): [M+H]⁺=608.3.

### Step 5: Synthesis of N-[3-(4-methoxy-1H-indazol-5-yl)-1H-pyrrolo[2,3-b]pyridin-6-yl]cyclopropanecarboxamide (Compound 16)

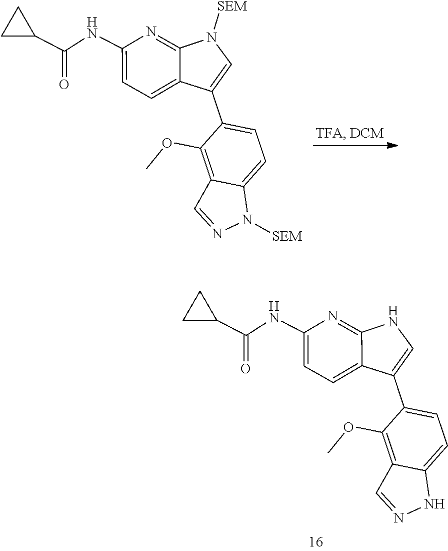 [SMILES: COc1c(-c2cn(COCC[Si](C)(C)C)c3nc(NC(=O)C4CC4)ccc23)ccc2c1cnn2COCC[Si](C)(C)C ; COc1c(-c2c[nH]c3nc(NC(=O)C4CC4)ccc23)ccc2[nH]ncc12]

To a mixture of N-[3-(4-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropanecarboxamide (190.0 mg, 0.31 mmol) in DCM (3.0 mL) was added TFA (1.0 mL). The resulting mixture was stirred at room temperature for 2 h. The resulting mixture was concentrated under reduced pressure. The residue was re-dissolved in CH₃CN/H₂O (5.0/1.0 mL). Then K₂CO₃ (500.0 mg. 3.62 mmol) was added to the mixture. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×150 mm, 5 um; Mobile Phase A: Water (10 mmol/L NH₄HCO₃), Mobile Phase B: ACN; Flow rate: 60 mL/min; Gradient: 28% B to 48% B in 7 min; 254 nm) to afford N-[3-(4-methoxy-1H-indazol-5-yl)-1H-pyrrolo[2,3-b]pyridin-6-yl]cyclopropanecarboxamide (17.1 mg, 15%) as a white solid. LCMS (ESI, m/z): [M+H]⁺=348.1. ¹H NMR (300 MHz, DMSO-d₆): δ 13.22 (s, 1H), 11.35 (s, 1H), 10.45 (s, 1H), 8.43 (s, 1H), 7.88-7.74 (m, 3H), 7.26 (d, J=8.7 Hz, 1H), 6.77 (d, J=2.1 Hz, 1H), 4.25 (s, 3H), 2.11-2.02 (m, 1H), 0.87-0.79 (m, 4H).

### Example S17. Compound 17

### Step 1: Synthesis of 5-methoxy-6-(4,4,5,5-tetramethyl-1,3,2-dioxaborolan-2-yl)-1H-indole

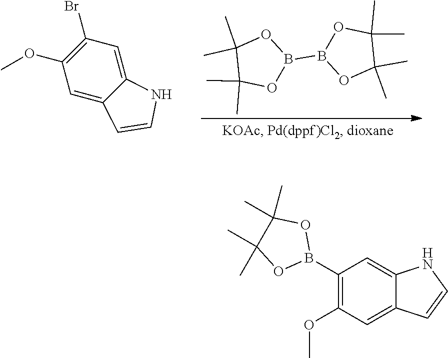

To a solution of 6-bromo-5-methoxy-1H-indole (1.0 g, 4.42 mmol) in 1,4-dioxane (8.0 mL) was added 4,4,4′,4′,5,5,5′,5′-octamethyl-2,2′-bi(1,3,2-dioxaborolane) (3.4 g, 13.27 mmol), KOAc (1.3 g, 13.22 mmol) and Pd(dppf)Cl₂ (0.4 g, 0.44 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 5-methoxy-6-(4,4,5,5-tetramethyl-1,3,2-dioxaborolan-2-yl)-1H-indole (900.0 mg, 74%) as a white solid. LCMS (ESI, m/z): [M+H]⁺=274.2.

### Step 2: Synthesis of 6-(6-chloro-1-[[2-(trimethylsilyl)ethoxy]methyl]pyrrolo[2,3-b]pyridin-3-yl)-5-methoxy-1H-indole

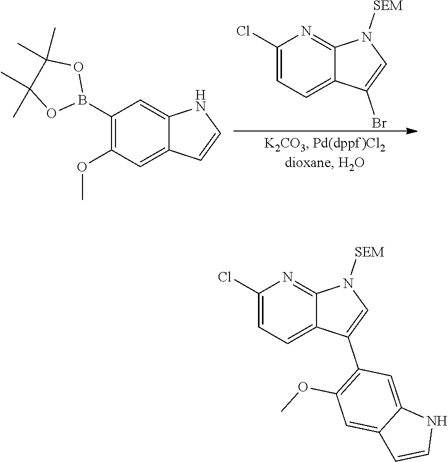 [SMILES: COc1cc2cc[nH]c2cc1B1OC(C)(C)C(C)(C)O1 ; C[Si](C)(C)CCOCn1cc(Br)c2ccc(Cl)nc21 ; COc1cc2cc[nH]c2cc1-c1cn(COCC[Si](C)(C)C)c2nc(Cl)ccc12]

To a solution of 5-methoxy-6-(4,4,5,5-tetramethyl-1,3,2-dioxaborolan-2-yl)-1H-indole (350.0 mg, 1.28 mmol) in 1,4-dioxane/H₂O (5.0/1.0 mL) was added 3-bromo-6-chloro-1-[[2-(trimethylsilyl)ethoxy]methyl]pyrrolo[2,3-b]pyridine (579.4 mg, 1.28 mmol), K₂CO₃ (531.3 mg, 3.84 mmol) and Pd(dppf)Cl₂ (104.6 mg, 0.13 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (89/11, v/v) to afford 6-(6-chloro-1-[[2-(trimethylsilyl)ethoxy]methyl]pyrrolo[2,3-b]pyridin-3-yl)-5-methoxy-1H-indole (200.0 mg, 36%) as a white solid. LCMS (ESI, m/z): [M+H]⁺=428.1.

### Step 3: Synthesis of N-[3-(5-methoxy-1H-indol-6-yl)-1-[[2-(trimethylsilyl)ethoxy]methyl]pyrrolo[2,3-b]pyridin-6-yl]cyclopropanecarboxamide

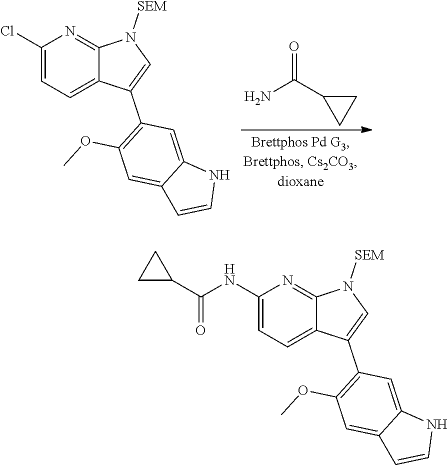

To a solution of 6-(6-chloro-1-[[2-(trimethylsilyl)ethoxy]methyl]pyrrolo[2,3-b]pyridin-3-yl)-5-methoxy-1H-indole (180.0 mg, 0.42 mmol) in dioxane (3.0 mL) was added cyclopropanecarboxamide (143.1 mg, 1.68 mmol), BrettPhos (90.3 mg, 0.17 mmol), Cs₂CO₃ (411.1 mg, 1.26 mmol) and BrettPhos Pd G3 (76.2 mg, 0.08 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (81/19, v/v) to afford N-[3-(5-methoxy-1H-indol-6-yl)-1-[[2-(trimethylsilyl)ethoxy]methyl]pyrrolo[2,3-b]pyridin-6-yl]cyclopropanecarboxamide (159.0 mg, 79%) as a white solid. LCMS (ESI, m/z): [M+H]⁺=477.2.

### Step 4: Synthesis of N-[3-(5-methoxy-1H-indol-6-yl)-1H-pyrrolo[2,3-b]pyridin-6-yl]cyclopropanecarboxamide (Compound 17)

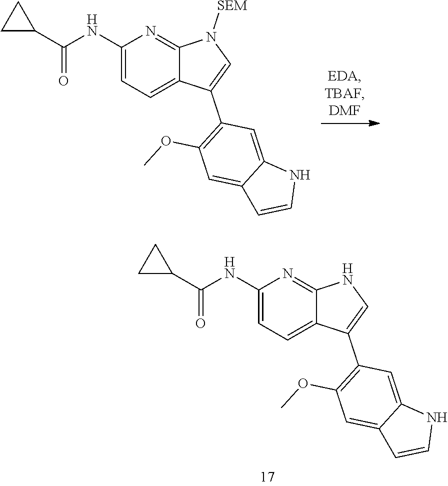 [SMILES: COc1cc2cc[nH]c2cc1-c1cn(COCC[Si](C)(C)C)c2nc(NC(=O)C3CC3)ccc12 ; COc1cc2cc[nH]c2cc1-c1c[nH]c2nc(NC(=O)C3CC3)ccc12]

To a solution of N-[3-(5-methoxy-1H-indol-6-yl)-1-[[2-(trimethylsilyl)ethoxy]methyl]pyrrolo[2,3-b]pyridin-6-yl]cyclopropanecarboxamide (150.0 mg, 0.31 mmol) in DMF (2.0 mL) was added EDA (94.5 mg, 1.57 mmol) and TBAF (246.8 mg, 0.94 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 27% B to 54% B in 8 min; 254 nm) to afford N-[3-(5-methoxy-1H-indol-6-yl)-1H-pyrrolo[2,3-b]pyridin-6-yl]cyclopropanecarboxamide (14.9 mg, 14%) as a white solid. LCMS (ESI, m/z): [M+H]⁺=347.2. ¹H NMR (400 MHz, DMSO-d₆): δ 11.43 (s, 1H), 10.85 (s, 1H), 10.56 (s, 1H), 7.96 (d, J=8.8 Hz, 1H), 7.88 (d, J=8.8 Hz, 1H), 7.50 (s, 2H), 7.28 (s, 1H), 7.18 (s, 1H), 6.37 (s, 1H), 3.78 (s, 3H), 2.07-2.03 (m, 1H), 0.83-0.77 (m, 4H).

### Example S18. Compound 18

### Step 1: Synthesis of 5-(6-chloro-1-[[2-(trimethylsilyl)ethoxy]methyl]pyrrolo[2,3-b]pyridin-3-yl)-1H-indole

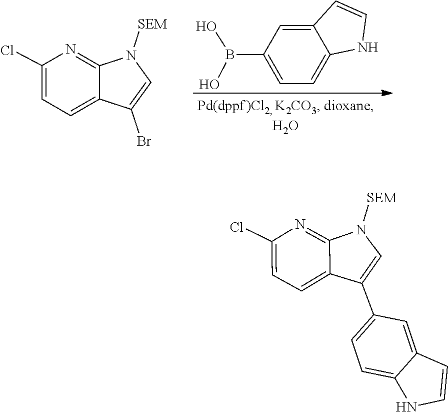 [SMILES: C[Si](C)(C)CCOCn1cc(Br)c2ccc(Cl)nc21 ; OB(O)c1ccc2[nH]ccc2c1 ; C[Si](C)(C)CCOCn1cc(-c2ccc3[nH]ccc3c2)c2ccc(Cl)nc21]

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (5.0/1.0 mL) was added 1H-indol-5-ylboronic acid (222.5 mg, 1.38 mmol), K₂CO₃ (573.1 mg, 4.15 mmol) and Pd(dppf)Cl₂ (225.7 mg, 0.27 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (84/16, v/v) to afford 5-(6-chloro-1-[[2-(trimethylsilyl)ethoxy]methyl]pyrrolo[2,3-b]pyridin-3-yl)-1H-indole (220.0 mg, 40%) as a white solid. LCMS (ESI, m/z): [M+H]⁺=398.1.

### Step 2: Synthesis of N-[3-(1H-indol-5-yl)-1-[[2-(trimethylsilyl)ethoxy]methyl]pyrrolo[2,3-b]pyridin-6-yl]cyclopropanecarboxamide

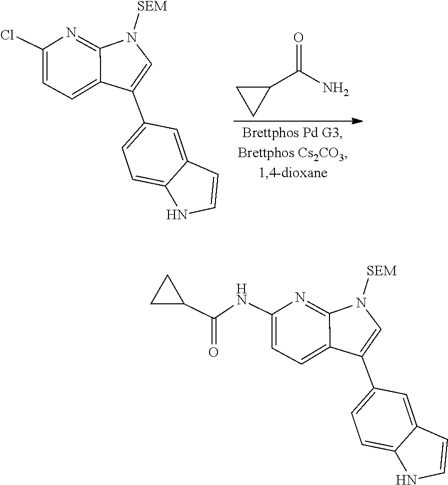 [SMILES: C[Si](C)(C)CCOCn1cc(-c2ccc3[nH]ccc3c2)c2ccc(Cl)nc21 ; NC(=O)C1CC1 ; C[Si](C)(C)CCOCn1cc(-c2ccc3[nH]ccc3c2)c2ccc(NC(=O)C3CC3)nc21]

To a solution of 5-(6-chloro-1-[[2-(trimethylsilyl)ethoxy]methyl]pyrrolo[2,3-b]pyridin-3-yl)-1H-indole (200.0 mg, 0.50 mmol) in dioxane (3.0 mL) was added cyclopropanecarboxamide (128.3 mg, 1.51 mmol), BrettPhos (107.9 mg, 0.20 mmol), Cs₂CO₃ (491.2 mg, 1.51 mmol) and BrettPhos Pd G3 (91.1 mg, 0.10 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (79/21, v/v) to afford N-[3-(1H-indol-5-yl)-1-[[2-(trimethylsilyl)ethoxy]methyl]pyrrolo[2,3-b]pyridin-6-yl]cyclopropanecarboxamide (150.0 mg, 66%) as a white solid. LCMS (ESI, m/z): [M+H]⁺=447.2.

### Step 3: Synthesis of N-[3-(1H-indol-5-yl)-1H-pyrrolo[2,3-b]pyridin-6-yl]cyclopropanecarboxamide (Compound 18)

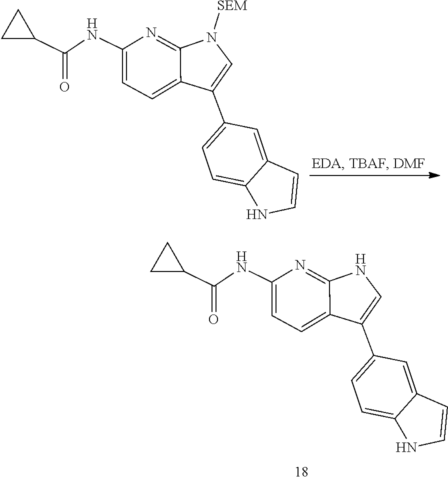 [SMILES: C[Si](C)(C)CCOCn1cc(-c2ccc3[nH]ccc3c2)c2ccc(NC(=O)C3CC3)nc21 ; O=C(Nc1ccc2c(-c3ccc4[nH]ccc4c3)c[nH]c2n1)C1CC1]

To a solution of N-[3-(1H-indol-5-yl)-1-[[2-(trimethylsilyl)ethoxy]methyl]pyrrolo[2,3-b]pyridin-6-yl]cyclopropanecarboxamide (100.0 mg, 0.22 mmol) in DMF (2.0 mL) was added EDA (40.4 mg, 0.67 mmol) and TBAF (292.7 mg, 1.12 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 27% B to 49% B in 9 min; 254 nm) to afford N-[3-(1H-indol-5-yl)-1H-pyrrolo[2,3-b]pyridin-6-yl]cyclopropanecarboxamide (26.7 mg, 38%) as a white solid. LCMS (ESI, m/z): [M+H]⁺=317.2. ¹H NMR (400 MHz, DMSO-d₆): δ 11.45 (s, 1H), 11.04 (s, 1H), 10.58 (s, 1H), 8.22 (d, J=8.4 Hz, 1H), 7.92 (d, J=8.8 Hz, 1H), 7.83 (s, 1H), 7.58 (d, J=1.6 Hz, 1H), 7.46-7.41 (m, 2H), 7.33 (d, J=2.4 Hz, 1H), 6.46 (s, 1H), 2.07-2.03 (m, 1H), 0.83-0.79 (m, 4H).

### Example S19. Compound 19

### Step 1: Synthesis of 5-(6-chloro-1-[[2-(trimethylsilyl)ethoxy]methyl]pyrrolo[2,3-b]pyridin-3-yl)-11H-1,3-benzodiazole

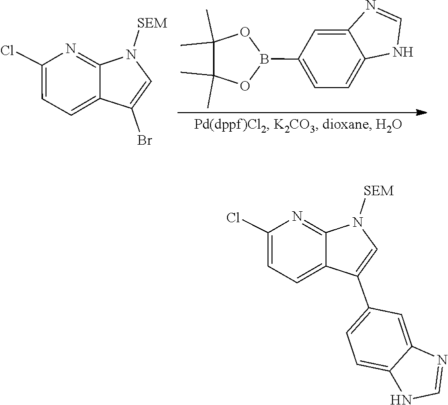

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (5.0/1.0 mL) was added 5-(4,4,5,5-tetramethyl-1,3,2-dioxaborolan-2-yl)-1H-1,3-benzodiazole (404.9 mg, 1.66 mmol), K₂CO₃ (573.1 mg, 4.15 mmol) and Pd(dppf)Cl₂ (112.9 mg, 0.14 mmol) at room temperature under N₂. The resulting mixture was stirred at 80° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ethyl acetate/MeOH (10/1, v/v) to afford 5-(6-chloro-1-[[2-(trimethylsilyl)ethoxy]methyl]pyrrolo[2,3-b]pyridin-3-yl)-1H-1,3-benzodiazole (280.0 mg, 50%) as a white solid. LCMS (ESI, m/z): [M+H]⁺=399.1.

### Step 2: Synthesis of N-[3-(1H-1,3-benzodiazol-5-yl)-1-[[2-(trimethylsilyl)ethoxy]methyl]pyrrolo[2,3-b]pyridin-6-yl]cyclopropanecarboxamide

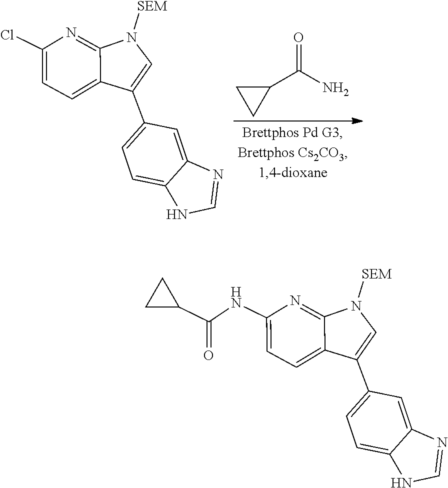 [SMILES: C[Si](C)(C)CCOCn1cc(-c2ccc3[nH]cnc3c2)c2ccc(Cl)nc21 ; NC(=O)C1CC1 ; C[Si](C)(C)CCOCn1cc(-c2ccc3[nH]cnc3c2)c2ccc(NC(=O)C3CC3)nc21]

To a solution of 5-(6-chloro-1-[[2-(trimethylsilyl)ethoxy]methyl]pyrrolo[2,3-b]pyridin-3-yl)-1H-1,3-benzodiazole (280.0 mg, 0.70 mmol) in 1,4-dioxane (10.0 mL) was added cyclopropanecarboxamide (1194.5 mg, 14.04 mmol), BrettPhos (75.34 mg, 0.14 mmol), Cs₂CO₃ (686.0 mg, 2.10 mmol) and BrettPhos Pd G3 (63.6 mg, 0.07 mmol) at room temperature under N₂. The resulting mixture was irradiated with microwave radiation at 120° C. for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ethyl acetate/MeOH (10/1, v/v) to afford N-[3-(1H-1,3-benzodiazol-5-yl)-1-[[2-(trimethylsilyl)ethoxy]methyl]pyrrolo[2,3-b]pyridin-6-yl]cyclopropanecarboxamide (250.0 mg, 79%) as a white solid. LCMS (ESI, m/z): [M+H]⁺=448.2.

### Step 3: Synthesis of N-[3-(1H-1,3-benzodiazol-5-yl)-1H-pyrrolo[2,3-b]pyridin-6-yl]cyclopropanecarboxamide (Compound 19)

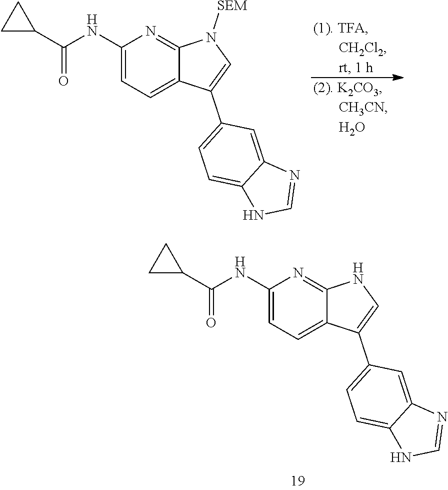 [SMILES: C[Si](C)(C)CCOCn1cc(-c2ccc3[nH]cnc3c2)c2ccc(NC(=O)C3CC3)nc21 ; O=C(Nc1ccc2c(-c3ccc4[nH]cnc4c3)c[nH]c2n1)C1CC1]

To a solution of N-[3-(1H-1,3-benzodiazol-5-yl)-1-[[2-(trimethylsilyl)ethoxy]methyl]pyrrolo[2,3-b]pyridin-6-yl]cyclopropanecarboxamide (400.0 mg, 0.89 mmol) in CH₂Cl₂ (5.0 mL) was added TFA (5.0 mL) at room temperature. The resulting mixture was stirred at room temperature for 3 h. The reaction mixture was evaporated in vacuo. The residue was re-dissolved in ACN/H₂O (10.0/2.0 mL). Then K₂CO₃ (1235.0 mg, 8.94 mmol) was added to the mixture. The mixture was stirred at room temperature for another 16 h. After the reaction was completed, the mixture was filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 15% B to 45% B in 10 min; 254 nm) to afford N-[3-(1H-1,3-benzodiazol-5-yl)-1H-pyrrolo[2,3-b]pyridin-6-yl]cyclopropanecarboxamide (9.0 mg, 3%) as a white solid. LCMS (ESI, m/z): [M+H]⁺=318.1. ¹H NMR (300 MHz, DMSO-d₆): δ 12.41-12.35 (m, 1H), 11.59-11.55 (m, 1H), 10.60 (s, 1H), 8.25-8.19 (m, 2H), 7.97-7.54 (m, 5H), 2.08-2.03 (m, 1H), 0.84-0.79 (m, 4H).

### Example S20. Compound 20

### Step 1: Synthesis of 6-chloro-3-(1H-indol-6-yl)-1-((2-(trimethylsilyl)ethoxy)methyl)-1H-pyrrolo[2,3-b]pyridine

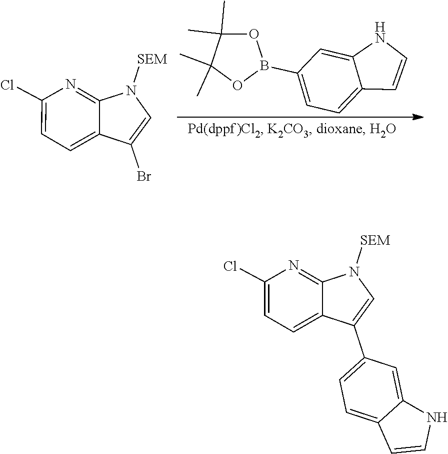

To a solution of 3-bromo-6-chloro-1-((2-(trimethylsilyl)ethoxy)methyl)-1H-pyrrolo[2,3-b]pyridine (500.0 mg, 1.38 mmol) in dioxane/H₂O (8.0/2.0 mL) was added 6-(4,4,5,5-tetramethyl-1,3,2-dioxaborolan-2-yl)-1H-indole (336.0 mg, 1.38 mmol), K₂CO₃ (573.1 mg, 4.15 mmol) and Pd(dppf)Cl₂ (101.1 mg, 0.14 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 6-chloro-3-(1H-indol-6-yl)-1-((2-(trimethylsilyl)ethoxy)methyl)-1H-pyrrolo[2,3-b]pyridine (160.0 mg, 29%) as a white solid. LCMS (ESI, m/z): [M+H]⁺=398.1.

### Step 2: Synthesis of N-(3-(1H-indol-6-yl)-1-((2-(trimethylsilyl)ethoxy)methyl)-1H-pyrrolo[2,3-b]pyridin-6-yl)cyclopropanecarboxamide

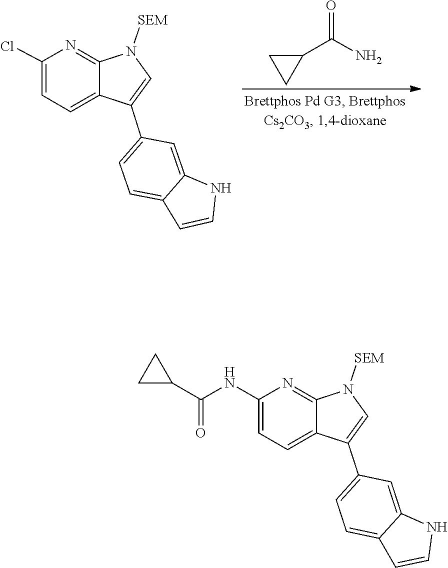 [SMILES: C[Si](C)(C)CCOCn1cc(-c2ccc3cc[nH]c3c2)c2ccc(Cl)nc21 ; NC(=O)C1CC1 ; C[Si](C)(C)CCOCn1cc(-c2ccc3cc[nH]c3c2)c2ccc(NC(=O)C3CC3)nc21]

To a solution of 6-chloro-3-(1H-indol-6-yl)-1-((2-(trimethylsilyl)ethoxy)methyl)-1H-pyrrolo[2,3-b]pyridine (160.0 mg, 0.40 mmol) in 1,4-dioxane (10.0 mL) was added cyclopropanecarboxamide (684.3 mg, 8.04 mmol), Cs₂CO₃ (393.0 mg, 1.21 mmol) and Brettphos (43.2 mg, 0.08 mmol), BrettPhos Pd G3 (36.4 mg, 0.04 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (6/1, v/v) to afford N-(3-(1H-indol-6-yl)-1-((2-(trimethylsilyl)ethoxy)methyl)-1H-pyrrolo[2,3-b]pyridin-6-yl)cyclopropanecarboxamide (130.0 mg, 72%) as a white solid. LCMS (ESI, m/z): [M+H]⁺=447.2.

### Step 3: Synthesis of N-(3-(1H-indol-6-yl)-1H-pyrrolo[2,3-b]pyridin-6-yl)cyclopropanecarboxamide (Compound 20)

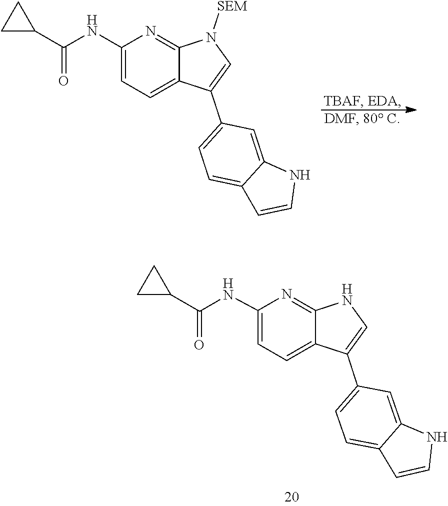 [SMILES: C[Si](C)(C)CCOCn1cc(-c2ccc3cc[nH]c3c2)c2ccc(NC(=O)C3CC3)nc21 ; O=C(Nc1ccc2c(-c3ccc4cc[nH]c4c3)c[nH]c2n1)C1CC1]

To a solution of N-(3-(1H-indol-6-yl)-1-((2-(trimethylsilyl)ethoxy)methyl)-1H-pyrrolo[2,3-b]pyridin-6-yl)cyclopropanecarboxamide (100.0 mg, 0.22 mmol) in DMF (5.0 mL) was added TBAF (0.6 mL) and EDA (67.2 mg, 1.12 mmol). The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with 5-100% CH₃CN in H₂O and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 30% B to 57% B in 8 min; 220 nm) to afford N-(3-(1H-indol-6-yl)-1H-pyrrolo[2,3-b]pyridin-6-yl)cyclopropanecarboxamide (1.4 mg, 1%) as a white solid. LCMS (ESI, m/z): [M+H]⁺=317.2. ¹H NMR (300 MHz, DMSO-d₆): δ 11.53 (s, 1H), 11.01 (s, 1H), 10.61 (s, 1H), 8.23 (d, J=9.0 Hz, 1H), 7.95 (d, J=8.4 Hz, 1H), 7.68-7.63 (m, 2H), 7.59 (d, J=8.1 Hz, 1H), 7.36-7.33 (m, 2H), 6.43 (s, 1H), 2.08-1.98 (m, 1H), 0.84-0.79 (m, 4H).

### Example S21. Compound 21

### Step 1: Synthesis of tert-butyl 5-(6-chloro-1-((2-(trimethylsilyl)ethoxy)methyl)-1H-pyrrolo[2,3-b]pyridin-3-yl)-1H-indazole-1-carboxylate

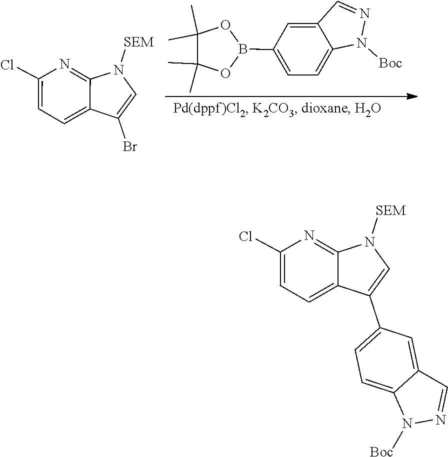

To a solution of 3-bromo-6-chloro-1-[[2-(trimethylsilyl)ethoxy]methyl]pyrrolo[2,3-b]pyridine (500.0 mg, 1.38 mmol) in dioxane/H₂O (8.0/2.0 mL) was added tert-butyl 5-(4,4,5,5-tetramethyl-1,3,2-dioxaborolan-2-yl)indazole-1-carboxylate (475.8 mg, 1.38 mmol), K₂CO₃ (573.1 mg, 4.15 mmol) and Pd(dppf)Cl₂ (101.1 mg, 0.14 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford tert-butyl 5-(6-chloro-1-((2-(trimethylsilyl)ethoxy)methyl)-1H-pyrrolo[2,3-b]pyridin-3-yl)-1H-indazole-1-carboxylate (140.0 mg, 20%) as a colorless oil. LCMS (ESI, m/z): [M+H]⁺=499.2.

### Step 2: Synthesis of tert-butyl 5-(6-(cyclopropanecarboxamido)-1-((2-(trimethylsilyl)ethoxy)methyl)-1H-pyrrolo[2,3-b]pyridin-3-yl)-1H-indazole-1-carboxylate

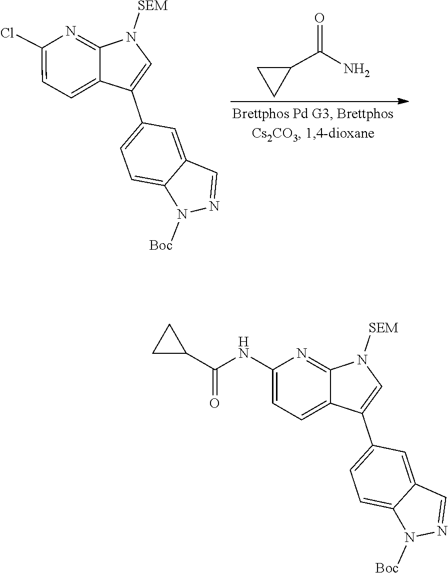 [SMILES: CC(C)(C)OC(=O)n1ncc2cc(-c3cn(COCC[Si](C)(C)C)c4nc(Cl)ccc34)ccc21 ; NC(=O)C1CC1 ; CC(C)(C)OC(=O)n1ncc2cc(-c3cn(COCC[Si](C)(C)C)c4nc(NC(=O)C5CC5)ccc34)ccc21]

To a solution of tert-butyl 5-(6-chloro-1-[[2-(trimethylsilyl)ethoxy]methyl]pyrrolo[2,3-b]pyridin-3-yl)indazole-1-carboxylate (110.0 mg, 0.22 mmol) in 1,4-dioxane (10.0 mL) was added cyclopropanecarboxamide (375.2 mg, 4.41 mmol), Cs₂CO₃ (215.4 mg, 0.66 mmol), Brettphos (23.7 mg, 0.04 mmol) and BrettPhos Pd G3 (20.0 mg, 0.02 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford tert-butyl 5-(6-(cyclopropanecarboxamido)-1-((2-(trimethylsilyl)ethoxy)methyl)-1H-pyrrolo[2,3-b]pyridin-3-yl)-1H-indazole-1-carboxylate (110.0 mg, 91%) as a white solid. LCMS (ESI, m/z): [M+H]⁺=548.3.

### Step 3: Synthesis of N-(3-(1H-indazol-5-yl)-1H-pyrrolo[2,3-b]pyridin-6-yl)cyclopropanecarboxamide (Compound 21)

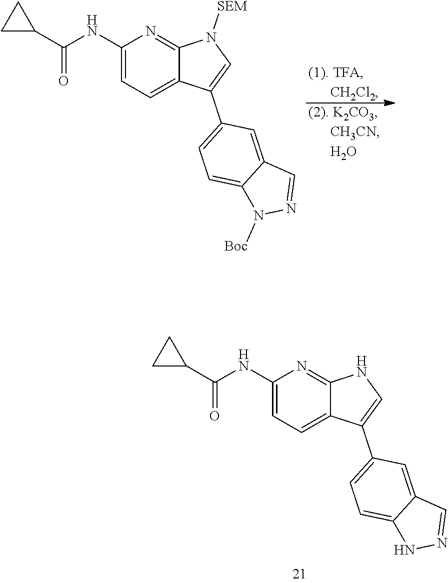 [SMILES: CC(C)(C)OC(=O)n1ncc2cc(-c3cn(COCC[Si](C)(C)C)c4nc(NC(=O)C5CC5)ccc34)ccc21 ; O=C(Nc1ccc2c(-c3ccc4[nH]ncc4c3)c[nH]c2n1)C1CC1]

To a solution of tert-butyl 5-(6-(cyclopropanecarboxamido)-1-((2-(trimethylsilyl)ethoxy)methyl)-1H-pyrrolo[2,3-b]pyridin-3-yl)-1H-indazole-1-carboxylate (110.0 mg, 0.20 mmol) in CH₂Cl₂ (5.0 mL) was added TFA (5.0 mL). The resulting mixture was stirred at room temperature for 3 h. The resulting mixture was concentrated under reduced pressure. The residue was re-dissolved in CH₃CN/H₂O (20.0/20.0 mL). Then K₂CO₃ (277.6 mg, 2.00 mmol) was added to the mixture. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 25% B to 39% B in 8 min; 254 nm) to afford N-[3-(1H-indazol-5-yl)-1H-pyrrolo[2,3-b]pyridin-6-yl]cyclopropanecarboxamide (9.3 mg, 14%) as a white solid. LCMS (ESI, m/z): [M+H]⁺=318.2. ¹H NMR (400 MHz, DMSO-d₆): δ 13.02 (s, 1H), 11.58 (s, 1H), 10.62 (s, 1H), 8.29 (d, J=8.8 Hz, 1H), 8.09-8.05 (m, 2H), 7.96 (d, J=8.8 Hz, 1H), 7.74-7.71 (m, 2H), 7.60 (d, J=8.8 Hz, 1H), 2.07-1.99 (m, 1H), 0.85-0.80 (m, 4H).

### Example S22. Compound 22

### Step 1: Synthesis of 6-chloro-3-(2-fluoro-6-methoxyphenyl)-1-[[2-(trimethylsilyl)ethoxy]methyl]pyrrolo[2,3-b]pyridine

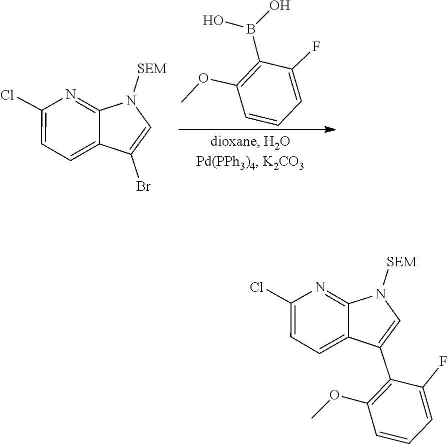

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (8.0/2.0 mL) was added 2-fluoro-6-methoxyphenylboronic acid (281.9 mg, 1.66 mmol), K₂CO₃ (382.1 mg, 2.76 mmol) and Pd(PPh₃)₄ (159.7 mg, 0.14 mmol) at room temperature under N₂. The resulting mixture was stirred at 100° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 6-chloro-3-(2-fluoro-6-methoxyphenyl)-1-[[2-(trimethylsilyl)ethoxy]methyl]pyrrolo[2,3-b]pyridine (220.0 mg, 39%) as a brown oil. LCMS (ESI, m/z): [M+H]⁺=407.1.

### Step 2: Synthesis of (1R,2R)-2-fluoro-N-[3-(2-fluoro-6-methoxyphenyl)-1-[[2-(trimethylsilyl)ethoxy]methyl]pyrrolo[2,3-b]pyridin-6-yl]cyclopropane-1-carboxamide

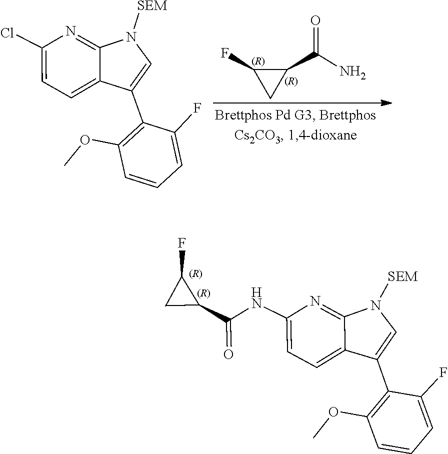

To a solution of 6-chloro-3-(2-fluoro-6-methoxyphenyl)-1-[[2-(trimethylsilyl)ethoxy]methyl]pyrrolo[2,3-b]pyridine (250.0 mg, 0.61 mmol) in 1,4-dioxane (6.0 mL) was added (1R,2R)-2-fluorocyclopropane-1-carboxamide (316.7 mg, 3.07 mmol), BrettPhos (66.0 mg, 0.12 mmol), Cs₂CO₃ (600.5 mg, 1.84 mmol) and BrettPhos Pd G3 (55.7 mg, 0.06 mmol) at room temperature. The resulting mixture was irradiated with microwave radiation at 120° C. for 1.5 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ether/ethyl acetate (3/2, v/v) to afford (1R,2R)-2-fluoro-N-[3-(2-fluoro-6-methoxyphenyl)-1-[[2-(trimethylsilyl)ethoxy]methyl]pyrrolo[2,3-b]pyridin-6-yl]cyclopropane-1-carboxamide (146.0 mg, 50%) as a brown yellow oil. LCMS (ESI, m/z): [M+H]⁺=474.2.

### Step 3: Synthesis of (1R,2R)-2-fluoro-N-[3-(2-fluoro-6-methoxyphenyl)-1H-pyrrolo[2,3-b]pyridin-6-yl]cyclopropane-1-carboxamide (Compound 22)

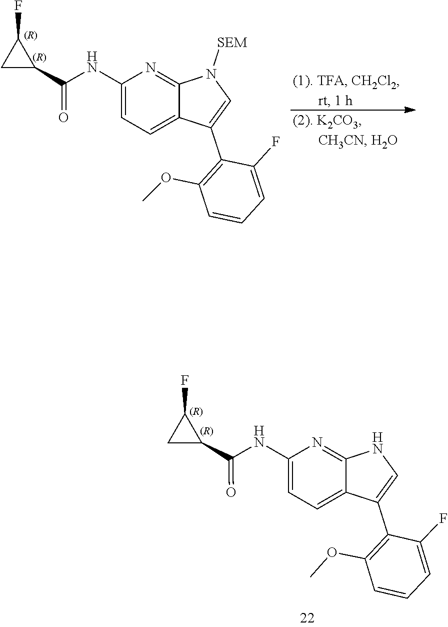

To a solution of (1R,2R)-2-fluoro-N-[3-(2-fluoro-6-methoxyphenyl)-1-[[2-(trimethylsilyl)ethoxy]methyl]pyrrolo[2,3-b]pyridin-6-yl]cyclopropane-1-carboxamide (146.0 mg, 0.31 mmol) in CH₂Cl₂ (5.0 mL) was added TFA (5.0 mL) at room temperature. The resulting mixture was stirred at room temperature for 3 h. The mixture was evaporated under reduced pressure. The residue was re-dissolved in ACN/H₂O (10.0/2.0 mL). Then K₂CO₃ (426.1 mg, 3.08 mmol) was added to the mixture. The mixture was stirred at room temperature for another 16 h. After the reaction was completed, the reaction mixture was filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 31% B to 61% B in 10 min; 254 nm) to afford (1R,2R)-2-fluoro-N-[3-(2-fluoro-6-methoxyphenyl)-1H-pyrrolo[2,3-b]pyridin-6-yl]cyclopropane-1-carboxamide (10.8 mg, 10%) as a white solid. LCMS (ESI, m/z): [M+H]⁺=344.1. ¹H NMR (400 MHz, DMSO-d₆): δ 11.67 (s, 1H), 10.63 (s, 1H), 7.88-7.86 (m, 1H), 7.70-7.68 (m, 1H), 7.46 (s, 1H), 7.37-7.31 (m, 1H), 6.99-6.90 (m, 2H), 5.03-4.82 (m, 1H), 3.78 (s, 3H), 2.27-2.20 (m, 1H), 1.71-1.63 (m, 1H), 1.20-1.05 (m, 1H).

### Example S23. Compound 23

### Step 1: Synthesis of 2,2-difluorocyclopropane-1-carboxamide

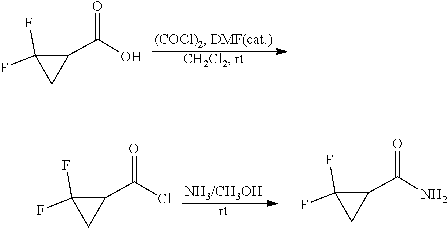 [SMILES: O=C(O)C1CC1(F)F ; O=C(Cl)C1CC1(F)F ; NC(=O)C1CC1(F)F]

To a solution of 2,2-difluorocyclopropane-1-carboxylic acid (800.0 mg, 6.55 mmol) in DCM (10.0 mL) was added DMF (0.1 mL) and oxalyl chloride (998.1 mg, 7.86 mmol). The resulting mixture was stirred at room temperature for 1 h. Then NH₃ (g)/MeOH (10.0 mL, 7 mol/L) was added to the mixture. The mixture was stirred at room temperature for another 0.5 h. After the reaction was completed, the reaction mixture was concentrated under reduced pressure to afford 2,2-difluorocyclopropane-1-carboxamide (790.0 mg, crude) as a white solid. LCMS (ESI, m/z): [M+H]⁺=122.0.

### Step 2: Synthesis of 6-chloro-3-(2-methoxyphenyl)-1-[[2-(trimethylsilyl)ethoxy]methyl]pyrrolo[2,3-b]pyridine

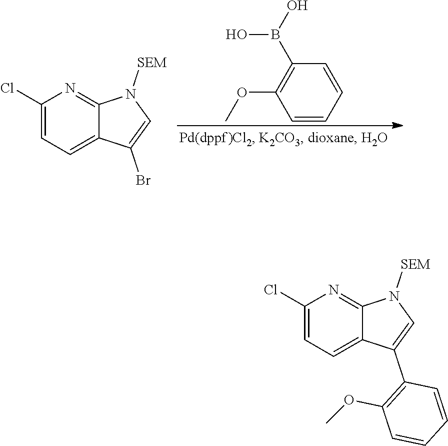 [SMILES: C[Si](C)(C)CCOCn1cc(Br)c2ccc(Cl)nc21 ; COc1ccccc1B(O)O ; COc1ccccc1-c1cn(COCC[Si](C)(C)C)c2nc(Cl)ccc12]

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (20.0/4.0 mL) was added 2-methoxyphenylboronic acid (252.1 mg, 1.66 mmol), K₂CO₃ (573.1 mg, 4.15 mmol) and Pd(dppf)Cl₂ (112.9 mg, 0.14 mmol). The resulting mixture was stirred at 80° C. for 16 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2-methoxyphenyl)-1-[[2-(trimethylsilyl)ethoxy]methyl]pyrrolo[2,3-b]pyridine (230.0 mg, 42%) as a colorless oil. LCMS (ESI, m/z): [M+H]⁺=389.1.

### Step 3: Synthesis of 2,2-difluoro-N-[3-(2-methoxyphenyl)-1-[[2-(trimethylsilyl)ethoxy]methyl]pyrrolo[2,3-b]pyridin-6-yl]cyclopropane-1-carboxamide

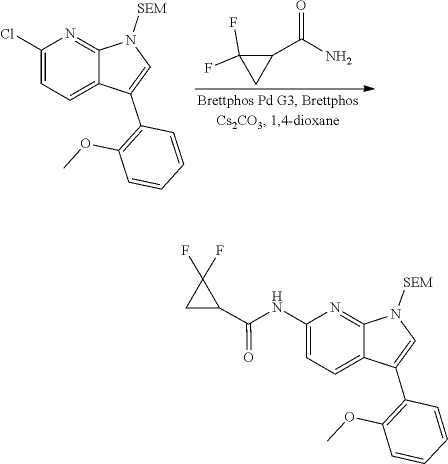 [SMILES: COc1ccccc1-c1cn(COCC[Si](C)(C)C)c2nc(Cl)ccc12 ; NC(=O)C1CC1(F)F ; COc1ccccc1-c1cn(COCC[Si](C)(C)C)c2nc(NC(=O)C3CC3(F)F)ccc12]

To a solution of 6-chloro-3-(2-methoxyphenyl)-1-[[2-(trimethylsilyl)ethoxy]methyl]pyrrolo[2,3-b]pyridine (170.0 mg, 0.44 mmol) in 1,4-dioxane (6.0 mL) was added 2,2-difluorocyclopropane-1-carboxamide (264.6 mg, 2.19 mmol), Cs₂CO₃ (427.2 mg, 1.31 mmol), BrettPhos (46.9 mg, 0.09 mmol) and BrettPhos Pd G3 (396.2 mg, 0.48 mmol). The resulting mixture was irradiated with microwave radiation at 120° C. for 1.5 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 2,2-difluoro-N-[3-(2-methoxyphenyl)-1-[[2-(trimethylsilyl)ethoxy]methyl]pyrrolo[2,3-b]pyridin-6-yl]cyclopropane-1-carboxamide (120.0 mg, 57%) as a brown yellow oil. LCMS (ESI, m/z): [M+H]⁺=474.2.

### Step 4: Synthesis of 2,2-difluoro-N-[3-(2-methoxyphenyl)-1H-pyrrolo[2,3-b]pyridin-6-yl]cyclopropane-1-carboxamide (Compound 23)

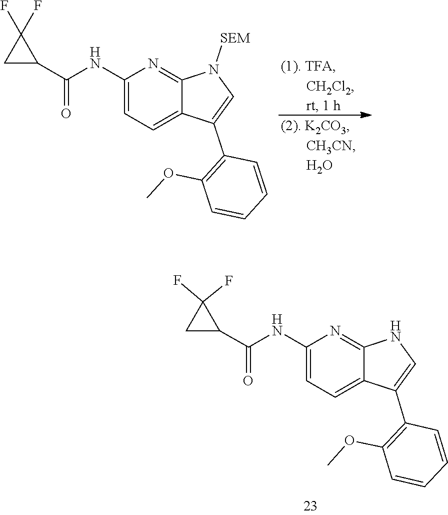 [SMILES: COc1ccccc1-c1cn(COCC[Si](C)(C)C)c2nc(NC(=O)C3CC3(F)F)ccc12 ; COc1ccccc1-c1c[nH]c2nc(NC(=O)C3CC3(F)F)ccc12]

To a solution of 2,2-difluoro-N-[3-(2-methoxyphenyl)-1-[[2-(trimethylsilyl)ethoxy]methyl]pyrrolo[2,3-b]pyridin-6-yl]cyclopropane-1-carboxamide (120.0 mg, 0.25 mmol) in CH₂Cl₂ (5.0 mL) was added TFA (5.0 mL). The resulting mixture was stirred at room temperature for 3 h. The reaction mixture was evaporated under reduced pressure. The residue was re-dissolved in ACN/H₂O (10.0/2.0 mL). Then K₂CO₃ (350.2 mg, 2.53 mmol) was added to the mixture. The mixture was stirred at room temperature for another 16 h. After the reaction was completed, the mixture was filtered. The filtrate was concentrated under reduced pressure. The residue was purified by Prep-HPLC with the following conditions (Column: XBridge Prep Phenyl OBD Column, 19×150 mm, 5 um, 13 nm; Mobile Phase A: Water (10 mmol/L NH₄HCO₃), Mobile Phase B: ACN; Flow rate: 60 mL/min; Gradient: 32% B to 62% B in 10 min; 254 nm) to afford 2,2-difluoro-N-[3-(2-methoxyphenyl)-1H-pyrrolo[2,3-b]pyridin-6-yl]cyclopropane-1-carboxamide (14.7 mg, 16%) as a white solid. LCMS (ESI, m/z): [M+H]⁺=344.1. ¹H NMR (400 MHz, DMSO-d₆): δ 11.64 (s, 1H), 10.82 (s, 1H), 8.02 (d, J=8.4 Hz, 1H), 7.88 (d, J=8.4 Hz, 1H), 7.60 (d, J=2.0 Hz, 1H), 7.54 (d, J=7.2 Hz, 1H), 7.30-7.26 (m, 1H), 7.12 (d, J=8.0 Hz, 1H), 7.05-7.01 (m, 1H), 3.82 (s, 3H), 3.06-2.98 (m, 1H), 2.09-1.95 (m, 2H).

### Example S24. Compound 24

### Step 1: Synthesis of tert-butyl 5-(4,4,5,5-tetramethyl-1,3,2-dioxaborolan-2-yl)-2,3-dihydroindole-1-carboxylate

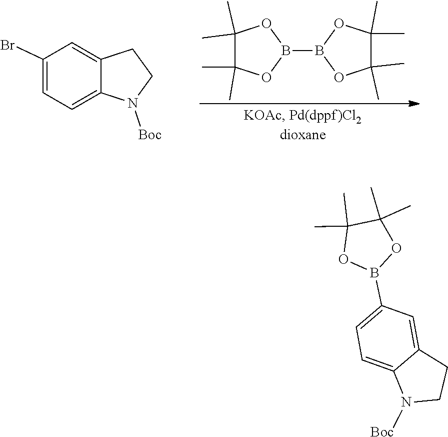

To a solution of tert-butyl 5-bromo-2,3-dihydroindole-1-carboxylate (1.0 g, 3.35 mmol) in 1,4-dioxane (10.0 mL) was added bis(pinacolato)diboron (2.6 g, 10.06 mmol), KOAc (987.4 mg, 10.06 mmol) and Pd(dppf)Cl₂ (112.6 mg, 0.15 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (89/11, v/v) to afford tert-butyl 5-(4,4,5,5-tetramethyl-1,3,2-dioxaborolan-2-yl)-2,3-dihydroindole-1-carboxylate (1.0 g, 73%) as a red solid. LCMS (ESI, m/z): [M+H]⁺=346.2.

### Step 2: Synthesis of tert-butyl 5-(6-chloro-1-[[2-(trimethylsilyl)ethoxy]methyl]pyrrolo[2,3-b]pyridin-3-yl)-2,3-dihydroindole-1-carboxylate

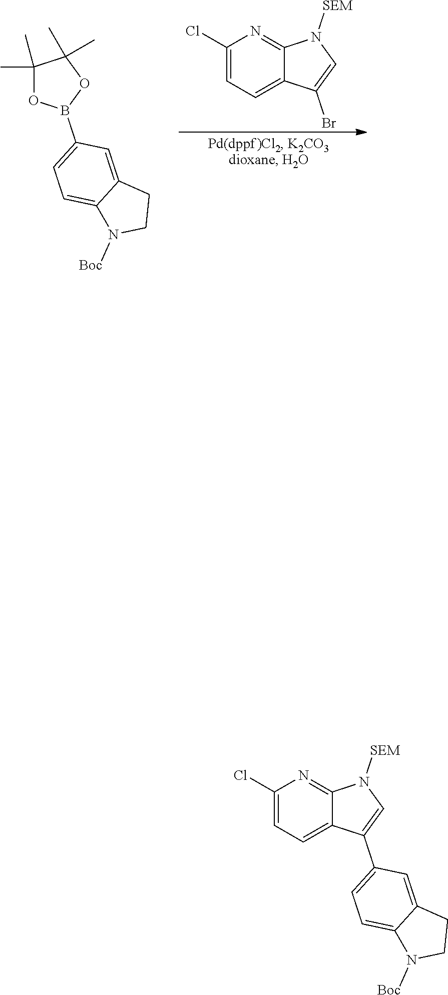 [SMILES: CC(C)(C)OC(=O)N1CCc2cc(B3OC(C)(C)C(C)(C)O3)ccc21 ; C[Si](C)(C)CCOCn1cc(Br)c2ccc(Cl)nc21 ; "Pd(dppf)Cl2, K2CO3dioxane, H2O" ; CC(C)(C)OC(=O)N1CCc2cc(-c3cn(COCC[Si](C)(C)C)c4nc(Cl)ccc34)ccc21]

To a solution of 3-bromo-6-chloro-1-[[2-(trimethylsilyl)ethoxy]methyl]pyrrolo[2,3-b]pyridine (360.0 mg, 0.99 mmol) in 1,4-dioxane/H₂O (5.0 mL/1.0 mL) was added tert-butyl 5-(4,4,5,5-tetramethyl-1,3,2-dioxaborolan-2-yl)-2,3-dihydroindole-1-carboxylate (412.3 mg, 1.19 mmol), K₂CO₃ (412.6 mg, 2.99 mmol), and Pd(dppf)Cl₂ (72.8 mg, 0.10 mmol) at room temperature under N₂. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (68/32, v/v) to afford tert-butyl 5-(6-chloro-1-[[2-(trimethylsilyl)ethoxy]methyl]pyrrolo[2,3-b]pyridin-3-yl)-2,3-dihydroindole-1-carboxylate (260.0 mg, 52%) as a yellow solid. LCMS (ESI, m/z): [M+H]⁺=500.2.

### Step 3: Synthesis of tert-butyl 5-(6-cyclopropaneamido-1-[[2-(trimethylsilyl)ethoxy]methyl]pyrrolo[2,3-b]pyridin-3-yl)-2,3-dihydroindole-1-carboxylate

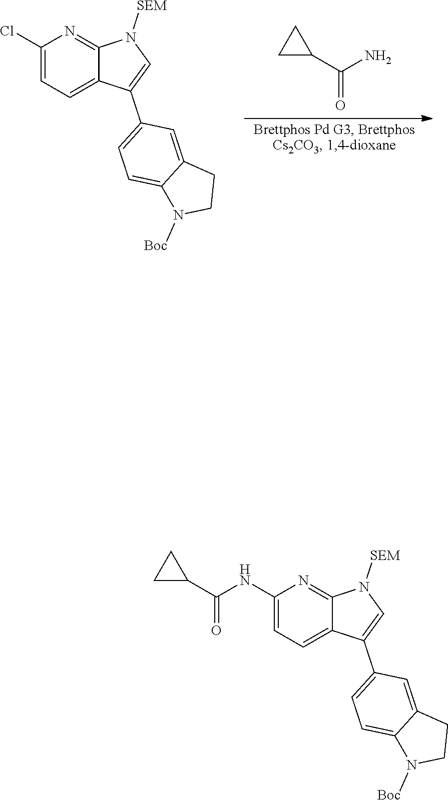

To a solution of tert-butyl 5-(6-chloro-1-[[2-(trimethylsilyl)ethoxy]methyl]pyrrolo[2,3-b]pyridin-3-yl)-2,3-dihydroindole-1-carboxylate (200.0 mg, 0.40 mmol) in 1,4-dioxane (5.0 mL) was added cyclopropanecarboxamide (40.8 mg, 0.48 mmol), Cs₂CO₃ (390.9 mg, 1.20 mmol), BrettPhos (42.9 mg, 0.08 mmol) and Brettphos Pd G3 (36.2 mg, 0.04 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (52/48, v/v) to afford tert-butyl 5-(6-cyclopropaneamido-1-[[2-(trimethylsilyl)ethoxy]methyl]pyrrolo[2,3-b]pyridin-3-yl)-2,3-dihydroindole-1-carboxylate (200.0 mg, 91%) as a yellow solid. LCMS (ESI, m/z): [M+H]⁺=549.2.

### Step 4: Synthesis of N-[3-(2,3-dihydro-1H-indol-5-yl)-1H-pyrrolo[2,3-b]pyridin-6-yl]cyclopropanecarboxamide (Compound 24)

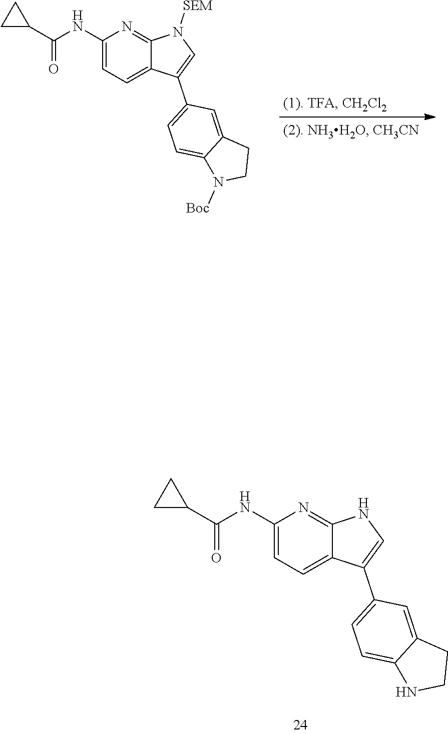 [SMILES: CC(C)(C)OC(=O)N1CCc2cc(-c3cn(COCC[Si](C)(C)C)c4nc(NC(=O)C5CC5)ccc34)ccc21 ; "(1). TFA, CH2Cl2" ; "(2). NH3·H2O, CH3CN" ; O=C(Nc1ccc2c(-c3ccc4c(c3)CCN4)c[nH]c2n1)C1CC1]

To a solution of tert-butyl 5-(6-cyclopropaneamido-1-[[2-(trimethylsilyl)ethoxy]methyl]pyrrolo[2,3-b]pyridin-3-yl)-2,3-dihydroindole-1-carboxylate (200.0 mg, 0.36 mmol) in CH₂Cl₂ (5.0 mL) was added TFA (5.0 mL). The mixture was stirred at room temperature for 1 h. The reaction mixture was evaporated in vacuo. The residue was re-dissolved in CH₃CN/NH₃·H₂O (5.0/5.0 mL). The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 18% B to 48% B in 10 min; 254 nm) to afford N-[3-(2,3-dihydro-1H-indol-5-yl)-1H-pyrrolo[2,3-b]pyridin-6-yl]cyclopropanecarboxamide (5.2 mg, 4%) as a white solid. LCMS (ESI, m/z): [M+H]⁺=319.2. ¹H NMR (300 MHz, DMSO-d₆): δ 11.38 (s, 1H), 10.57 (s, 1H), 8.14 (d, J=8.7 Hz, 1H), 7.89 (d, J=8.7 Hz, 1H), 7.47 (d, J=2.4 Hz, 1H), 7.37 (s, 1H), 7.25 (d, J=7.8 Hz, 1H), 6.57 (d, J=8.1 Hz, 1H), 5.47 (s, 1H), 3.47-3.42 (m, 2H), 3.00-2.97 (m, 2H), 2.08-1.92 (m, 1H), 0.83-0.78 (m, 4H).

### Example S25. Compound 25

### Step 1: Synthesis of tert-butyl 6-(4,4,5,5-tetramethyl-1,3,2-dioxaborolan-2-yl)-2,3-dihydroindole-1-carboxylate

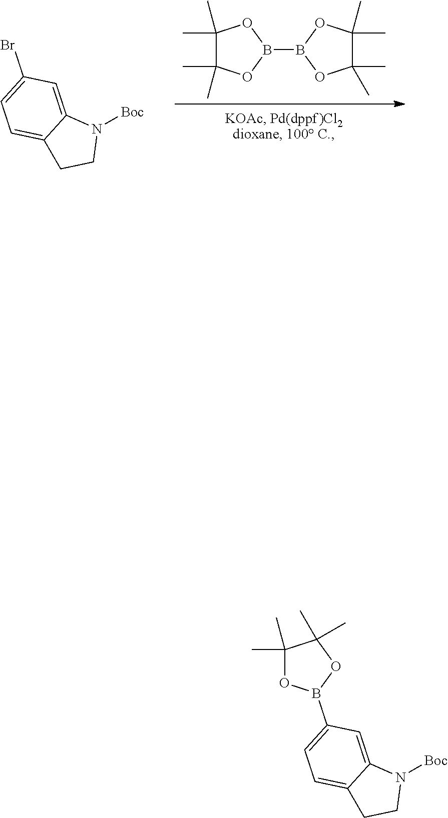

A mixture of tert-butyl 6-bromo-2,3-dihydroindole-1-carboxylate (1.0 g, 3.35 mmol), bis(pinacolato)diboron (1.3 g, 5.03 mmol), KOAc (1.0 g, 10.06 mmol) and Pd(dppf)Cl₂ (0.3 g, 0.34 mmol) in dioxane (25.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (89/11, v/v) to afford tert-butyl 6-(4,4,5,5-tetramethyl-1,3,2-dioxaborolan-2-yl)-2,3-dihydroindole-1-carboxylate (1.1 g, 95%) as a white solid. LCMS (ESI, m/z): [M+H]⁺=346.2.

### Step 2: Synthesis of tert-butyl 6-(6-chloro-1-[[2-(trimethylsilyl)ethoxy]methyl]pyrrolo[2,3-b]pyridin-3-yl)-2,3-dihydroindole-1-carboxylate

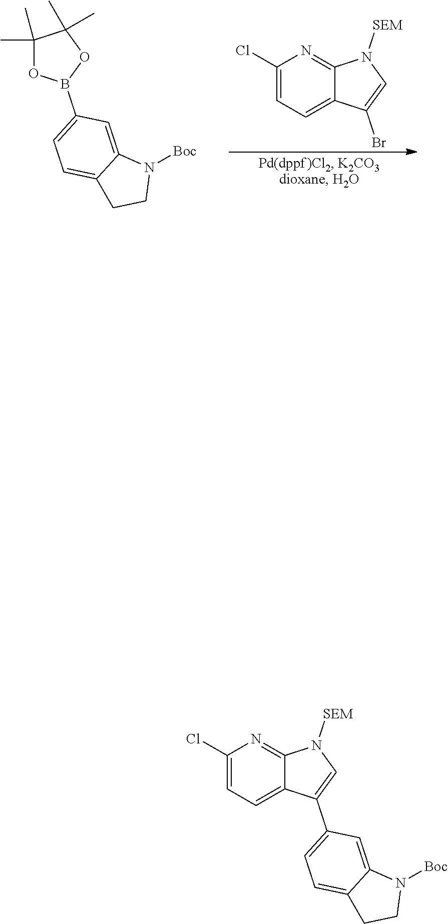 [SMILES: CC(C)(C)OC(=O)N1CCc2ccc(B3OC(C)(C)C(C)(C)O3)cc21 ; C[Si](C)(C)CCOCn1cc(Br)c2ccc(Cl)nc21 ; "Pd(dppf)Cl2, K2CO3dioxane, H2O" ; CC(C)(C)OC(=O)N1CCc2ccc(-c3cn(COCC[Si](C)(C)C)c4nc(Cl)ccc34)cc21]

To a solution of tert-butyl 6-(4,4,5,5-tetramethyl-1,3,2-dioxaborolan-2-yl)-2,3-dihydroindole-1-carboxylate (1.1 g, 3.19 mmol) in dioxane (20.0 mL) and H₂O (4.0 mL) was added 3-bromo-6-chloro-1-[[2-(trimethylsilyl)ethoxy]methyl]pyrrolo[2,3-b]pyridine (1.4 g, 3.87 mmol), K₂CO₃ (1.3 g, 9.42 mmol) and Pd(dppf)Cl₂ (0.2 g, 0.27 mmol). The mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (92/8, v/v) to afford tert-butyl 6-(6-chloro-1-[[2-(trimethylsilyl)ethoxy]methyl]pyrrolo[2,3-b]pyridin-3-yl)-2,3-dihydroindole-1-carboxylate (919.0 mg, 57%) as a white solid. LCMS (ESI, m/z): [M+H]⁺=500.2.

### Step 3: Synthesis of tert-butyl 6-(6-cyclopropaneamido-1-[[2-(trimethylsilyl)ethoxy]methyl]pyrrolo[2,3-b]pyridin-3-yl)-2,3-dihydroindole-1-carboxylate

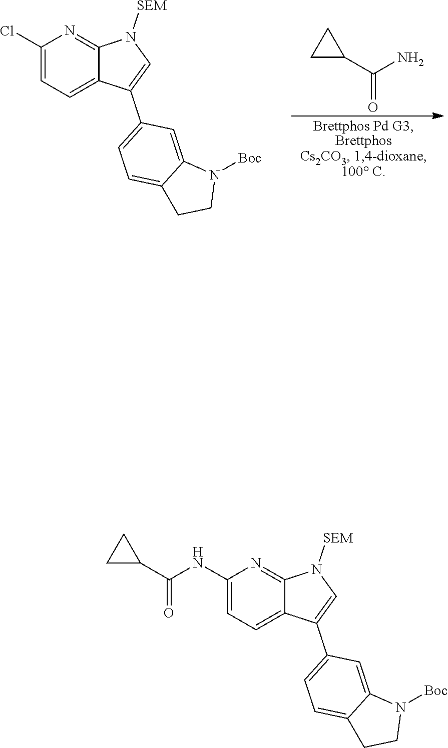

A mixture of tert-butyl 6-(6-chloro-1-[[2-(trimethylsilyl)ethoxy]methyl]pyrrolo[2,3-b]pyridin-3-yl)-2,3-dihydroindole-1-carboxylate (500.0 mg, 1.00 mmol), cyclopropanecarboxamide (255.3 mg, 3.00 mmol), Cs₂CO₃ (977.2 mg, 3.00 mmol), BrettPhos (107.3 mg, 0.20 mmol) and BrettPhos Pd G3 (90.6 mg, 0.10 mmol) in dioxane (10.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (71/29, v/v) to afford tert-butyl 6-(6-cyclopropaneamido-1-[[2-(trimethylsilyl)ethoxy]methyl]pyrrolo[2,3-b]pyridin-3-yl)-2,3-dihydroindole-1-carboxylate (437.4 mg, 79%) as a white solid. LCMS (ESI, m/z): [M+H]⁺=549.3.

### Step 4: Synthesis of N-[3-(2,3-dihydro-1H-indol-6-yl)-1H-pyrrolo[2,3-b]pyridin-6-yl]cyclopropanecarboxamide (Compound 25)

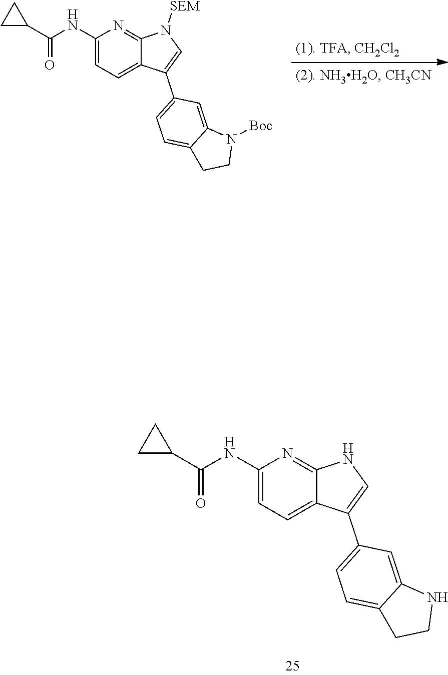 [SMILES: CC(C)(C)OC(=O)N1CCc2ccc(-c3cn(COCC[Si](C)(C)C)c4nc(NC(=O)C5CC5)ccc34)cc21 ; "(1). TFA, CH2Cl2" ; "(2). NH3·H2O, CH3CN" ; O=C(Nc1ccc2c(-c3ccc4c(c3)NCC4)c[nH]c2n1)C1CC1]

To a solution of tert-butyl 6-(6-cyclopropaneamido-1-[[2-(trimethylsilyl)ethoxy]methyl]pyrrolo[2,3-b]pyridin-3-yl)-2,3-dihydroindole-1-carboxylate (200.0 mg, 0.36 mmol) in CH₂Cl₂ (5.0 mL) was added TFA (5.0 mL). The mixture was stirred at room temperature for 2 h. The mixture was evaporated in vacuo. The residue was re-dissolved in CH₃CN (5.0 mL). Then NH₃·H₂O (5.0 mL) was add to the mixture. The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient 28% B to 49% B in 7 min; 254 nm; RT1:6.27 min) to afford N-[3-(2,3-dihydro-1H-indol-6-yl)-1H-pyrrolo[2,3-b]pyridin-6-yl]cyclopropanecarboxamide (13.1 mg, 11%) as a white solid. LCMS (ESI, m/z): [M+H]⁺=319.2. ¹H NMR (400 MHz, CD₃OD): δ 8.21-8.19 (m, 1H), 7.81 (d, J=8.8 Hz, 1H), 7.43 (s, 1H), 7.17 (d, J=7.2 Hz, 1H), 7.03-7.00 (m, 2H), 3.55-3.49 (m, 2H), 3.06-3.02 (m, 2H), 1.95-1.85 (m, 1H), 1.03-0.99 (m, 2H), 0.96-0.88 (m, 2H).

### Example S26. Compound 26

### Step 1: Synthesis of (1R,2R)-2-fluoro-N-(3-(2-methoxypyridin-3-yl)-1-((2-(trimethylsilyl)ethoxy)methyl)-1H-pyrrolo[2,3-b]pyridin-6-yl)cyclopropane-1-carboxamide

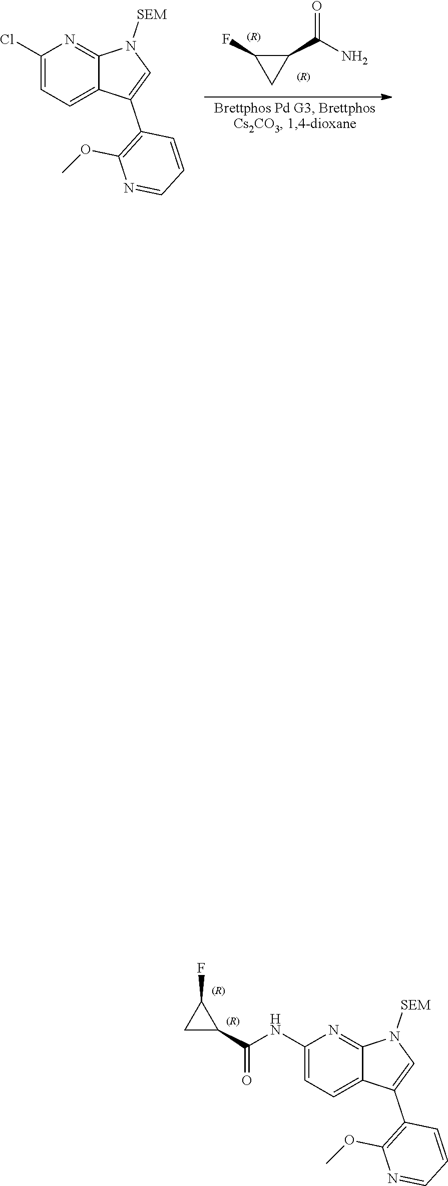

To a solution of 6-chloro-3-(2-methoxypyridin-3-yl)-1-((2-(trimethylsilyl)ethoxy)methyl)-1H-pyrrolo[2,3-b]pyridine (240.0 mg, 0.61 mmol) in dioxane (10.0 mL) were added Cs₂CO₃ (601.5 mg, 1.84 mmol), (1R,2R)-2-fluorocyclopropane-1-carboxamide (317.2 mg, 3.07 mmol), BrettPhos (132.1 mg, 0.24 mmol) and Brettphos Pd G3 (111.5 mg, 0.12 mmol) under N₂. The mixture was irradiated with microwave radiation at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ether/ethyl acetate (88/12, v/v) to afford (1R,2R)-2-fluoro-N-(3-(2-methoxypyridin-3-yl)-1-((2-(trimethylsilyl)ethoxy)methyl)-1H-pyrrolo[2,3-b]pyridin-6-yl)cyclopropane-1-carboxamide (230.0 mg, 81%) as a white solid. LCMS (ESI, m/z): [M+H]⁺=457.2.

### Step 2: Synthesis of (1R,2R)-2-fluoro-N-(3-(2-methoxypyridin-3-yl)-1H-pyrrolo[2,3-b]pyridin-6-yl)cyclopropane-1-carboxamide (Compound 26)

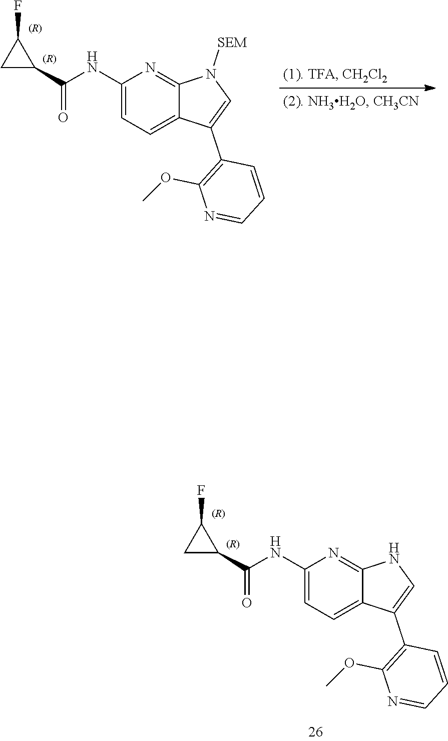

To a solution of (1R,2R)-2-fluoro-N-[3-(2-methoxypyridin-3-yl)-1-[[2-(trimethylsilyl)ethoxy]methyl]pyrrolo[2,3-b]pyridin-6-yl]cyclopropane-1-carboxamide (230.0 mg, 0.50 mmol) in CH₂Cl₂ (15.0 mL) was added TFA (15.0 mL). The resulting mixture was stirred at room temperature for 1 h. The resulting mixture was concentrated under vacuum. The residue was re-dissolved in CH₃CN (15.0 mL). Then NH₃H₂O (30.0 mL) was added to the mixture. The mixture was stirred at room temperature for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (80/20, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 25% B to 49% B in 8 min; 220 nm) to afford (1R,2R)-2-fluoro-N-(3-(2-methoxypyridin-3-yl)-1H-pyrrolo[2,3-b]pyridin-6-yl)cyclopropane-1-carboxamide (55.5 mg, 33%) as a white solid. LCMS (ESI, m/z): [M+H]⁺=327.2 ¹H NMR (300 MHz, DMSO-d₆): δ 11.70 (s, 1H), 10.64 (s, 1H), 8.13-8.07 (m, 2H), 8.01-7.90 (m, 2H), 7.72 (d, J=2.7 Hz, 1H), 7.10-7.06 (m, 1H), 5.05-4.80 (m, 1H), 3.95 (s, 3H), 2.26-2.21 (m, 1H), 1.71-1.61 (m, 1H), 1.19-1.08 (m, 1H).

### Example S27. Compound 27

### Step 1: Synthesis of 6-chloro-3-(2-methoxypyridin-3-yl)-1-((2-(trimethylsilyl)ethoxy)methyl)-1H-pyrrolo[2,3-b]pyridine

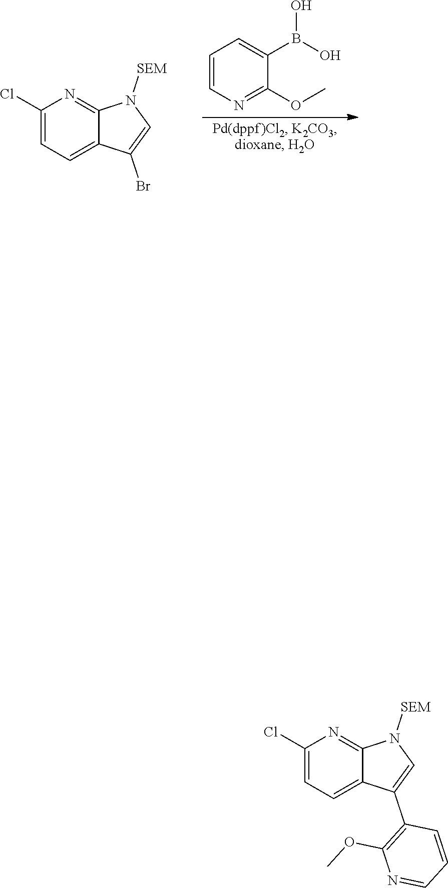

A mixture of 3-bromo-6-chloro-1-[[2-(trimethylsilyl)ethoxy]methyl]pyrrolo[2,3-b]pyridine (3.0 g, 8.29 mmol), 2-methoxypyridin-3-ylboronic acid (735.6 mg, 4.81 mmol), K₂CO₃ (2.0 g, 14.43 mmol) and Pd(dppf)Cl₂ (606.8 mg, 0.82 mmol) in dioxane (20.0 mL)/H₂O (2.0 mL) was stirred at 80° C. for 16 h under N₂. After the reaction was completed, the resulting mixture was concentrated under vacuum. The residue was purified by flash column chromatography with petroleum ether/ethyl acetate (7/3, v/v) to afford 3-(6-chloro-1-[[2-(trimethylsilyl)ethoxy]methyl]pyrrolo[2,3-b]pyridin-3-yl)-2-methoxypyridine (1400.0 mg, 43%) as a yellow oil. LCMS (ESI, m/z): [M+H]=390.0.

### Step 2: Synthesis of (1S,2S)-2-fluoro-N-(3-(2-methoxypyridin-3-yl)-1-((2-(trimethylsilyl)ethoxy)methyl)-1H-pyrrolo[2,3-b]pyridin-6-yl)cyclopropane-1-carboxamide

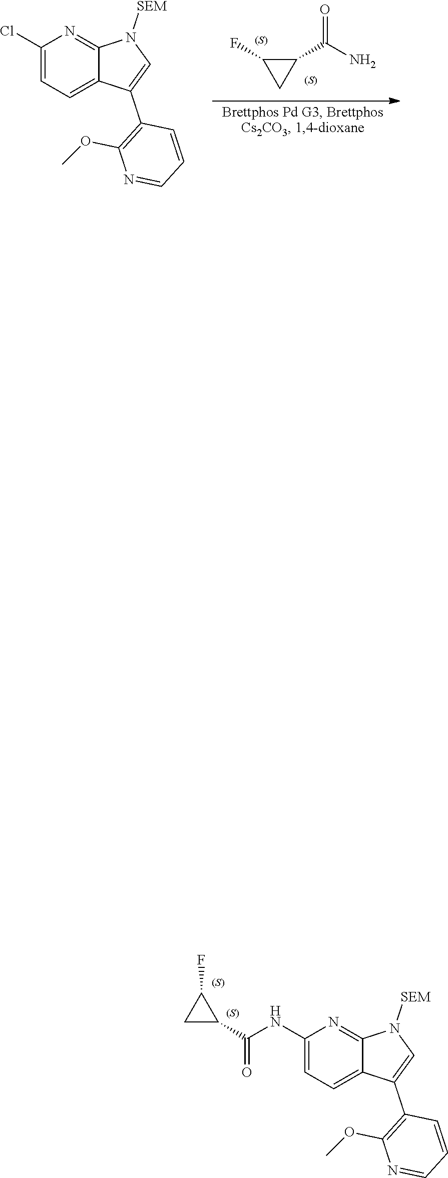

To a solution of 3-(6-chloro-1-[[2-(trimethylsilyl)ethoxy]methyl]pyrrolo[2,3-b]pyridin-3-yl)-2-methoxypyridine (400.0 mg, 1.02 mmol) in dioxane (8.0 mL) was added (1S,2S)-2-fluorocyclopropane-1-carboxamide (634.5 mg, 6.15 mmol), BrettPhos (220.2 mg, 0.41 mmol), Cs₂CO₃ (1002.6 mg, 3.07 mmol) and Brettphos Pd G3 (185.9 mg, 0.20 mmol). The final reaction mixture was irradiated with microwave radiation at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (7/3, v/v) to afford (1S,2S)-2-fluoro-N-[3-(2-methoxypyridin-3-yl)-1-[[2-(trimethylsilyl)ethoxy]methyl]pyrrolo[2,3-b]pyridin-6-yl]cyclopropane-1-carboxamide (125.0 mg, 26%) as a yellow oil. LCMS (ESI, m/z): [M+H]⁺=457.0.

### Step 3: Synthesis of (1S,2S)-2-fluoro-N-(3-(2-methoxypyridin-3-yl)-1H-pyrrolo[2,3-b]pyridin-6-yl)cyclopropane-1-carboxamide (Compound 27)

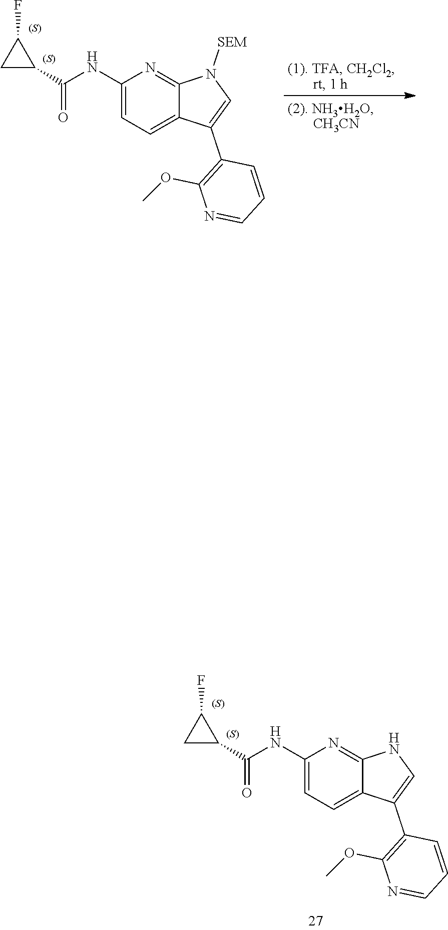

A mixture of (1S,2S)-2-fluoro-N-[3-(2-methoxypyridin-3-yl)-1-[[2-(trimethylsilyl)ethoxy]methyl]pyrrolo[2,3-b]pyridin-6-yl]cyclopropane-1-carboxamide (100.0 mg, 0.21 mmol) and TFA (5.0 mL) in CH₂Cl₂ (5.0 mL) was stirred at room temperature for 1 h. The resulting mixture was concentrated under vacuum. The residue was dissolved in CH₃CN (5.0 mL). Then NH₃H₂O (5.0 mL) was added to the mixture. The resulting mixture was stirred at room temperature for additional 1 h. After the reaction was completed, the resulting mixture was concentrated under vacuum. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 26% B to 46% B in 7 min; 220 nm) to afford (1S,2S)-2-fluoro-N-[3-(2-methoxypyridin-3-yl)-1H-pyrrolo[2,3-b]pyridin-6-yl]cyclopropane-1-carboxamide (18.8 mg, 26%) as a white solid. LCMS (ESI, m/z): [M+H]⁺=327.2. ¹H NMR (300 MHz, DMSO-d₆): δ 11.72 (s, 1H), 10.65 (s, 1H), 8.14-8.08 (m, 2H), 8.02-7.91 (m, 2H), 7.74 (d, J=2.4 Hz, 1H), 7.11-7.07 (m, 1H), 5.05-4.81 (m, 1H), 3.96 (s, 3H), 2.27-2.22 (m, 1H), 1.71-1.62 (m, 1H), 1.20-1.13 (m, 1H).

### Example S28. Compound 28

### Step 1: Synthesis of 5-(6-chloro-1-[[2-(trimethylsilyl)ethoxy]methyl]pyrrolo[2,3-b]pyridin-3-yl)-11H-1,3-benzodiazole

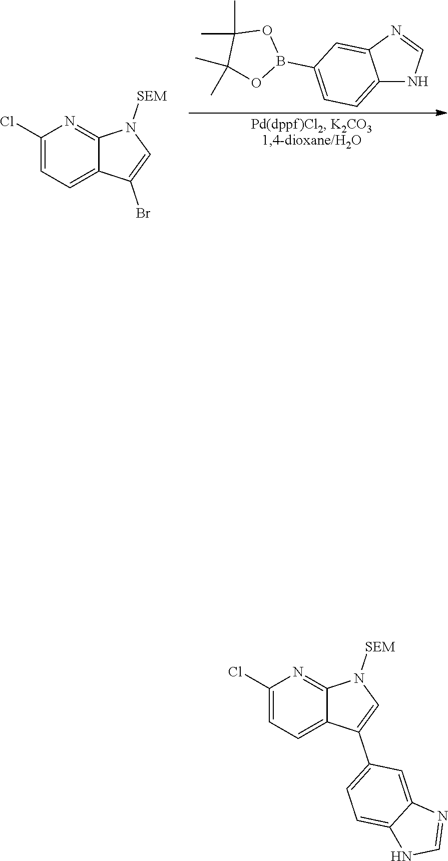

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (15.0/3.0 mL) was added 5-(4,4,5,5-tetramethyl-1,3,2-dioxaborolan-2-yl)-1H-1,3-benzodiazole (674.8 mg, 2.77 mmol), K₂CO₃ ((573.1 mg, 4.15 mmol) and Pd(dppf)Cl₂ (112.9 mg, 0.14 mmol) at room temperature under N₂. The resulting mixture was stirred at 100° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (12/1, v/v) to afford 5-(6-chloro-1-[[2-(trimethylsilyl)ethoxy]methyl]pyrrolo[2,3-b]pyridin-3-yl)-1H-1,3-benzodiazole (177.0 mg, 32%) as a yellow oil. LCMS (ESI, m/z): [M+H]⁺=399.1.

### Step 2: Synthesis of 5-(6-chloro-1-[[2-(trimethylsilyl)ethoxy]methyl]pyrrolo[2,3-b]pyridin-3-yl)-1-[[2-(trimethylsilyl)ethoxy]methyl]-1,3-benzodiazole

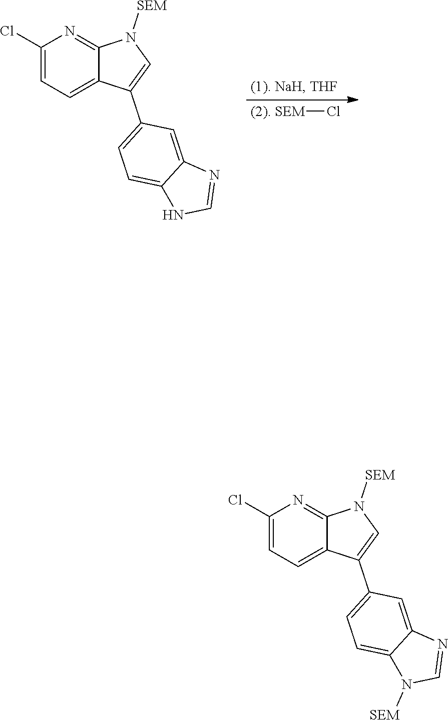

To a solution of 5-(6-chloro-1-[[2-(trimethylsilyl)ethoxy]methyl]pyrrolo[2,3-b]pyridin-3-yl)-1H-1,3-benzodiazole (297.0 mg, 0.74 mmol) in THF (20.0 mL) was added NaH (186.2 mg, 60%) at 0° C. under N₂. The resulting mixture was stirred at 0° C. for 1 h. Then SEM-Cl (186.2 mg, 1.12 mmol) was added dropwise to the mixture at 0° C. under N₂. The resulting mixture was stirred at 0° C. for another 1 h. After the reaction was completed, the reaction mixture was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 5-(6-chloro-1-[[2-(trimethylsilyl)ethoxy]methyl]pyrrolo[2,3-b]pyridin-3-yl)-1-[[2-(trimethylsilyl)ethoxy]methyl]-1,3-benzodiazole (200.0 mg, 50%) as a yellow oil. LCMS (ESI, m/z): [M+H]⁺=529.2.

### Step 3: Synthesis of (1S,2S)-2-fluoro-N-(1-[[2-(trimethylsilyl)ethoxy]methyl]-3-(1-[[2-(trimethylsilyl)ethoxy]methyl]-1,3-benzodiazol-5-yl)pyrrolo[2,3-b]pyridin-6-yl)cyclopropane-1-carboxamide

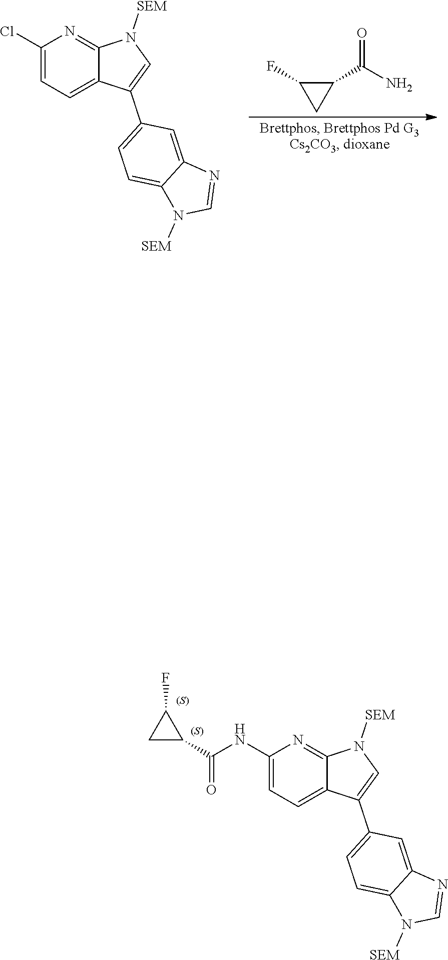

To a solution of 5-(6-chloro-1-[[2-(trimethylsilyl)ethoxy]methyl]pyrrolo[2,3-b]pyridin-3-yl)-1-[[2-(trimethylsilyl)ethoxy]methyl]-1,3-benzodiazole (189.0 mg, 0.36 mmol) in 1,4-dioxane (8.0 mL) was added (1S,2S)-2-fluorocyclopropane-1-carboxamide (184.1 mg, 1.79 mmol), BrettPhos (38.3 mg, 0.07 mmol), Cs₂CO₃ (349.1 mg, 1.07 mmol) and BrettPhos Pd G3 (32.4 mg, 0.04 mmol) at room temperature under N₂. The resulting mixture was irradiated with microwave radiation at 120° C. for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-(1-[[2-(trimethylsilyl)ethoxy]methyl]-3-(1-[[2-(trimethylsilyl)ethoxy]methyl]-1,3-benzodiazol-5-yl)pyrrolo[2,3-b]pyridin-6-yl)cyclopropane-1-carboxamide (166.0 mg, 78%) as a yellow solid. LCMS (ESI, m/z): [M+H]⁺=596.3.

### Step 4: Synthesis of (1S,2S)—N-[3-(1H-1,3-benzodiazol-5-yl)-1H-pyrrolo[2,3-b]pyridin-6-yl]-2-fluorocyclopropane-1-carboxamide (Compound 28)

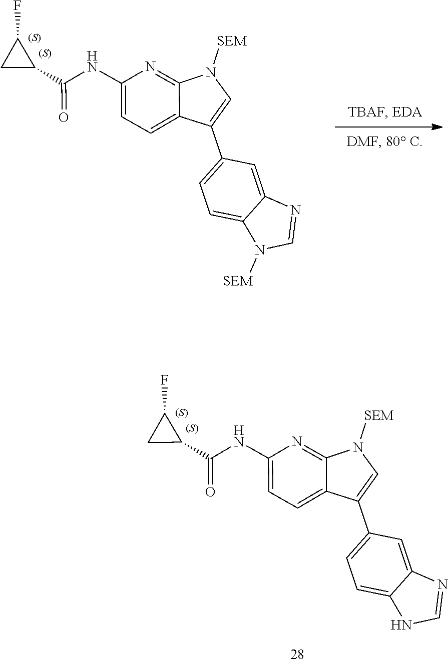

To a solution of (1S,2S)-2-fluoro-N-(1-[[2-(trimethylsilyl)ethoxy]methyl]-3-(1-[[2-(trimethylsilyl)ethoxy]methyl]-1,3-benzodiazol-5-yl)pyrrolo[2,3-b]pyridin-6-yl)cyclopropane-1-carboxamide (166.0 mg, 0.28 mmol) in DMF (8.0 mL) was added ethane-1,2-diamine (83.7 mg, 1.39 mmol) and TBAF (0.8 mL, 0.28 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 19% B to 39% B in 6 min; 254 nm) to (1S,2S)—N-[3-(1H-1,3-benzodiazol-5-yl)-1H-pyrrolo[2,3-b]pyridin-6-yl]-2-fluorocyclopropane-1-carboxamide (7.7 mg, 8%) as a white solid. LCMS (ESI, m/z): [M+H]⁺=336.1. ¹H NMR (300 MHz, DMSO-d₆): δ 12.40-12.35 (m, 1H), 11.63 (s, 1H), 10.78 (s, 1H), 8.26-8.19 (m, 2H), 7.91-7.89 (m, 2H), 7.77-7.70 (m, 2H), 7.56-7.54 (m, 1H), 5.01-4.78 (m, 1H), 2.59-2.50 (m, 1H), 1.54-1.46 (m, 1H), 1.28-1.22 (m, 1H).

### Example S29. Compound 29

### Step 1: Synthesis of 5-bromo-2-methyl-1-((2-(trimethylsilyl)ethoxy)methyl)-1H-benzo[d]imidazole

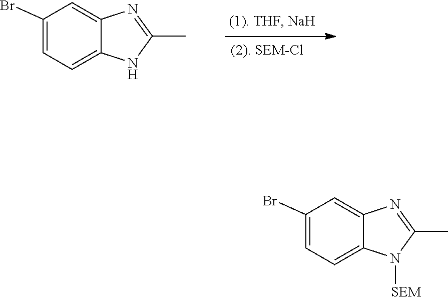 [SMILES: Cc1nc2cc(Br)ccc2[nH]1 ; Cc1nc2cc(Br)ccc2n1COCC[Si](C)(C)C]

To a solution of 5-bromo-2-methyl-1H-benzo[d]imidazole (500.0 mg, 2.37 mmol) in THF (20.0 mL) was added NaH (284.3 mg, 60%) at 0° C. under N₂. The resulting mixture was stirred at 0° C. for 1 h. Then SEM-Cl (592.4 mg, 3.55 mmol) was added dropwise to the mixture at 0° C. under N₂. The resulting mixture was stirred at 0° C. for another 1 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (63/37, v/v) to afford 5-bromo-2-methyl-1-((2-(trimethylsilyl)ethoxy)methyl)-1H-benzo[d]imidazole (710.0 mg, 87%) as a yellow solid. LCMS (ESI, m/z): [M+H]⁺=341.1.

### Step 2: Synthesis of 2-methyl-5-(4,4,5,5-tetramethyl-1,3,2-dioxaborolan-2-yl)-1-((2-(trimethylsilyl)ethoxy)methyl)-1H-benzo[d]imidazole

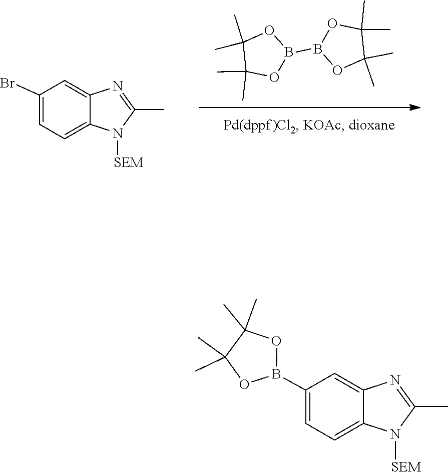

To a solution of 5-bromo-2-methyl-1-((2-(trimethylsilyl)ethoxy)methyl)-1H-benzo[d]imidazole (660.0 mg, 1.93 mmol) in 1,4-dioxane (15.0 mL) was added 4,4,4′,4′,5,5,5′,5′-octamethyl-2,2′-bi(1,3,2-dioxaborolane) (1473.1 mg, 5.80 mmol), KOAc (569.3 mg, 5.80 mmol) and Pd(dppf)Cl₂ (141.5 mg, 0.19 mmol) at room temperature under N₂. The resulting mixture was stirred at 80° C. for 16 h. After the reaction was completed, the resulting mixture was concentrated under reduced pressure. The residue was purified by flash column chromatography with petroleum ether/ethyl acetate (45/55, v/v) to afford 2-methyl-5-(4,4,5,5-tetramethyl-1,3,2-dioxaborolan-2-yl)-1-((2-(trimethylsilyl)ethoxy)methyl)-1H-benzo[d]imidazole (620.0 mg, 80%) as a light yellow solid. LCMS (ESI, m/z): [M+H]⁺=389.2.

### Step 3: Synthesis of 5-(6-chloro-1-((2-(trimethylsilyl)ethoxy)methyl)-1H-pyrrolo[2,3-b]pyridin-3-yl)-2-methyl-1-((2-(trimethylsilyl)ethoxy)methyl)-1H-benzo[d]imidazole

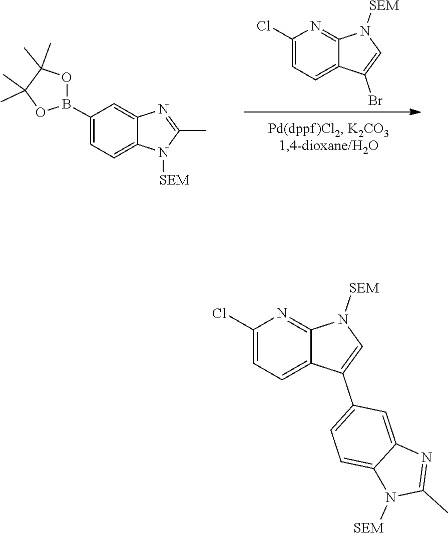

To a solution of 2-methyl-5-(4,4,5,5-tetramethyl-1,3,2-dioxaborolan-2-yl)-1-((2-(trimethylsilyl)ethoxy)methyl)-1H-benzo[d]imidazole (570.0 mg, 1.47 mmol) in 1,4-dioxane/H₂O (20.0/4.0 mL) was added 3-bromo-6-chloro-1-((2-(trimethylsilyl)ethoxy)methyl)-1H-pyrrolo[2,3-b]pyridine (530.9 mg, 1.47 mmol), K₂CO₃ (608.5 mg, 4.40 mmol) and Pd(dppf)Cl₂ (107.4 mg, 0.15 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (43/57, v/v) to afford 5-(6-chloro-1-((2-(trimethylsilyl)ethoxy)methyl)-1H-pyrrolo[2,3-b]pyridin-3-yl)-2-methyl-1-((2-(trimethylsilyl)ethoxy)methyl)-1H-benzo[d]imidazole (790.0 mg, 99%) as a yellow oil. LCMS (ESI, m/z): [M+H]⁺=543.2.

### Step 4: Synthesis of N-(3-(2-methyl-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide

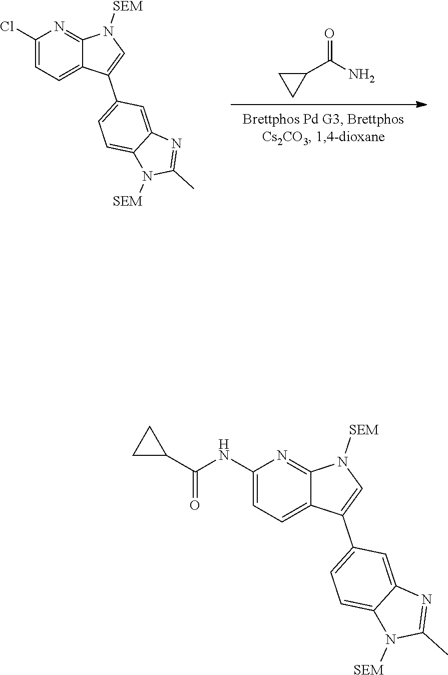 [SMILES: Cc1nc2cc(-c3cn(COCC[Si](C)(C)C)c4nc(Cl)ccc34)ccc2n1COCC[Si](C)(C)C ; NC(=O)C1CC1 ; Cc1nc2cc(-c3cn(COCC[Si](C)(C)C)c4nc(NC(=O)C5CC5)ccc34)ccc2n1COCC[Si](C)(C)C]

To a solution of 5-(6-chloro-1-((2-(trimethylsilyl)ethoxy)methyl)-1H-pyrrolo[2,3-b]pyridin-3-yl)-2-methyl-1-((2-(trimethylsilyl)ethoxy)methyl)-1H-benzo[d]imidazole (750.0 mg, 1.38 mmol) in 1,4-dioxane (15.0 mL) was added cyclopropanecarboxamide (587.5 mg, 6.90 mmol), Cs₂CO₃ (1349.4 mg, 4.14 mmol), BrettPhos (148.2 mg, 0.28 mmol) and BrettPhos Pd G3 (125.2 mg, 0.14 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (35/66, v/v) to afford N-(3-(2-methyl-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide (600.0 mg, 73%) as a light yellow solid. LCMS (ESI, m/z): [M+H]⁺=592.3.

### Step 5: Synthesis of N-(3-(2-methyl-1H-benzo[d]imidazol-5-yl)-1H-pyrrolo[2,3-b]pyridin-6-yl)cyclopropanecarboxamide (Compound 29)

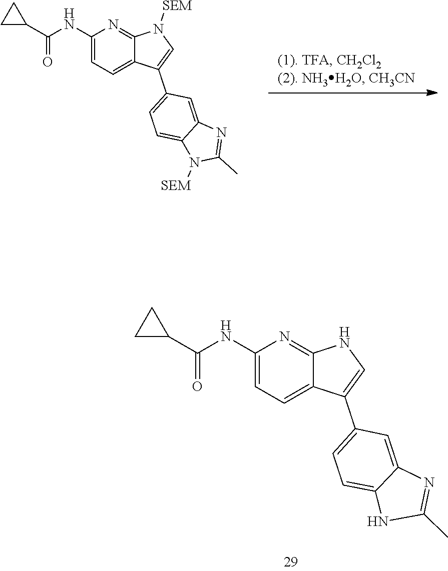 [SMILES: Cc1nc2cc(-c3cn(COCC[Si](C)(C)C)c4nc(NC(=O)C5CC5)ccc34)ccc2n1COCC[Si](C)(C)C ; Cc1nc2cc(-c3c[nH]c4nc(NC(=O)C5CC5)ccc34)ccc2[nH]1]

To a solution of N-(3-(2-methyl-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide (550.0 mg, 0.93 mmol) in CH₂Cl₂ (10.0 mL) was added TFA (10.0 mL) at room temperature. The resulting mixture was stirred at room temperature for 4 h. After the reaction was completed, the resulting mixture was concentrated under reduced pressure. The residue was dissolved in CH₃CN (10.0 mL and NH₃·H₂O (10.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 16% B to 40% B in 8 min; 254 nm) to afford N-(3-(2-methyl-1H-benzo[d]imidazol-5-yl)-1H-pyrrolo[2,3-b]pyridin-6-yl)cyclopropanecarboxamide (26.7 mg, 8%) as a white solid. LCMS (ESI, m/z): [M+H]⁺=332.2. ¹H NMR (300 MHz, CD₃OD): δ 8.25 (d, J=8.4 Hz, 1H), 7.85 (d, J=8.4 Hz, 1H), 7.74 (s, 1H), 7.57-7.51 (m, 3H), 2.60 (s, 3H), 1.94-1.89 (m, 1H), 1.05-0.96 (m, 2H), 0.94-0.91 (m, 2H).

### Example S30. Compound 30

### Step 1: Synthesis of 5-bromo-6-methoxy-N-methylpyridin-2-amine

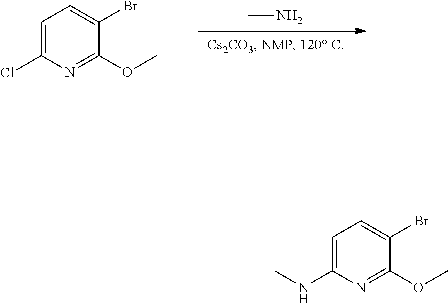

To a mixture of 3-bromo-6-chloro-2-methoxypyridine (1.0 g, 4.49 mmol) and methylamine (1.4 g, 45.07 mmol) in NMP (15.0 mL) was added Cs₂CO₃ (4.3 g, 13.47 mmol) at room temperature. The resulting mixture was stirred at 120° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 5-bromo-6-methoxy-N-methylpyridin-2-amine (950.0 mg, 97%) as a yellow oil. LCMS (ESI, m/z): [M+H]⁺=217.0.

### Step 2: Synthesis of 6-methoxy-N-methyl-5-(4,4,5,5-tetramethyl-1,3,2-dioxaborolan-2-yl)pyridin-2-amine

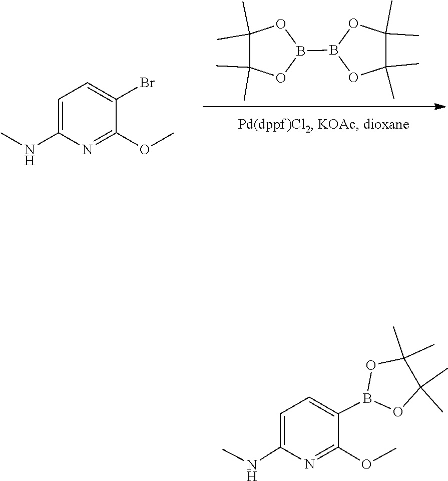

To a mixture of 5-bromo-6-methoxy-N-methylpyridin-2-amine (900.0 mg, 4.14 mmol) and bis(pinacolato)diboron (3.1 g, 12.43 mmol) in dioxane (20.0 mL) were added Pd(dppf)Cl₂ (303.3 mg, 0.41 mmol) and KOAc (1.2 g, 12.43 mmol) at room temperature. The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 6-methoxy-N-methyl-5-(4,4,5,5-tetramethyl-1,3,2-dioxaborolan-2-yl)pyridin-2-amine (180.0 mg, 16%) as a white solid. LCMS (ESI, m/z): [M+H]⁺=265.2.

### Step 3: Synthesis of 5-(6-chloro-1-[[2-(trimethylsilyl)ethoxy]methyl]pyrrolo[2,3-b]pyridin-3-yl)-6-methoxy-N-methylpyridin-2-amine

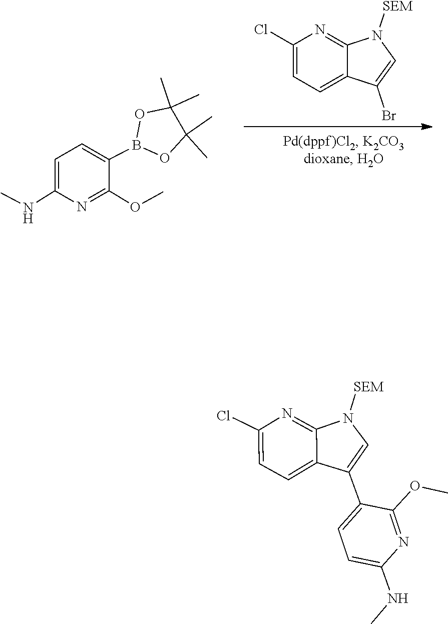

To a mixture of 6-methoxy-N-methyl-5-(4,4,5,5-tetramethyl-1,3,2-dioxaborolan-2-yl)pyridin-2-amine (160.0 mg, 0.62 mmol) and 3-bromo-6-chloro-1-[[2-(trimethylsilyl)ethoxy]methyl]pyrrolo[2,3-b]pyridine (150.0 mg, 0.41 mmol) in dioxane (4.0 mL) was added Pd(dppf)Cl₂ (30.0 mg, 0.041 mmol) and K₂CO₃ (170.0 mg, 2.08 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 5-(6-chloro-1-[[2-(trimethylsilyl)ethoxy]methyl]pyrrolo[2,3-b]pyridin-3-yl)-6-methoxy-N-methylpyridin-2-amine (70.0 mg, 28%) as a yellow solid. LCMS (ESI, m/z): [M+H]⁺=419.2.

### Step 4: Synthesis of N-[3-[2-methoxy-6-(methylamino)pyridin-3-yl]-1-[[2-(trimethylsilyl)ethoxy]methyl]pyrrolo[2,3-b]pyridin-6-yl]cyclopropanecarboxamide

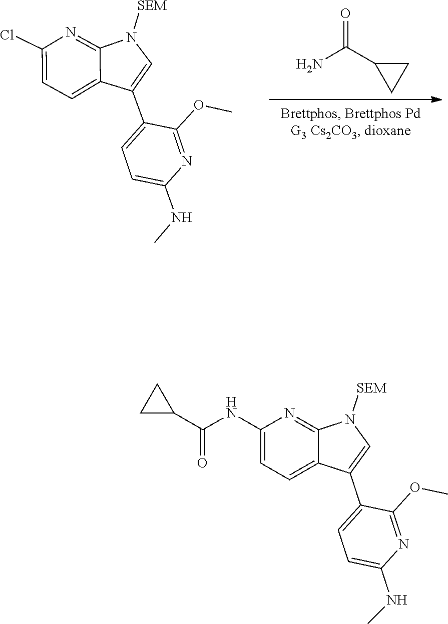 [SMILES: CNc1ccc(-c2cn(COCC[Si](C)(C)C)c3nc(Cl)ccc23)c(OC)n1 ; NC(=O)C1CC1 ; CNc1ccc(-c2cn(COCC[Si](C)(C)C)c3nc(NC(=O)C4CC4)ccc23)c(OC)n1]

To a mixture of 5-(6-chloro-1-[[2-(trimethylsilyl)ethoxy]methyl]pyrrolo[2,3-b]pyridin-3-yl)-6-methoxy-N-methylpyridin-2-amine (40.0 mg, 0.09 mmol) and cyclopropanecarboxamide (32.5 mg, 0.38 mmol) in dioxane (3.0 mL) was added BrettPhos (10.2 mg, 0.02 mmol) BrettPhos Pd G3 (8.6 mg, 0.01 mmol) and Cs₂CO₃ (93.3 mg, 0.28 mmol) at room temperature under N₂. The resulting mixture was stirred at 100° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford N-[3-[2-methoxy-6-(methylamino)pyridin-3-yl]-1-[[2-(trimethylsilyl)ethoxy]methyl]pyrrolo[2,3-b]pyridin-6-yl]cyclopropanecarboxamide (40.0 mg, 89%) as a yellow solid. LCMS (ESI, m/z): [M+H]⁺=468.2.

### Step 5: Synthesis of N-[3-[2-methoxy-6-(methylamino)pyridin-3-yl]-1H-pyrrolo[2,3-b]pyridin-6-yl]cyclopropanecarboxamide (Compound 30)

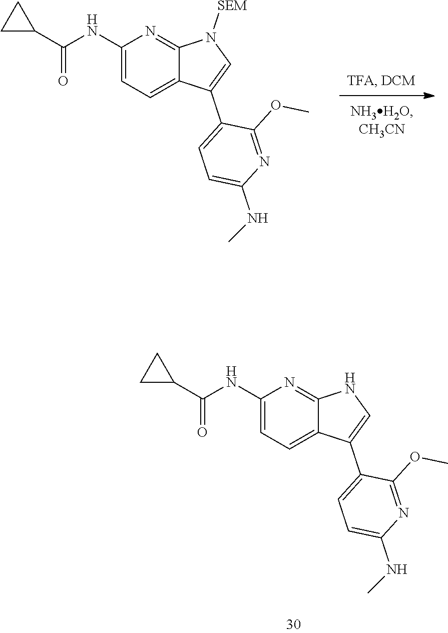 [SMILES: CNc1ccc(-c2cn(COCC[Si](C)(C)C)c3nc(NC(=O)C4CC4)ccc23)c(OC)n1 ; CNc1ccc(-c2c[nH]c3nc(NC(=O)C4CC4)ccc23)c(OC)n1]

To a solution of N-[3-[2-methoxy-6-(methylamino)pyridin-3-yl]-1-[[2-(trimethylsilyl)ethoxy]methyl]pyrrolo[2,3-b]pyridin-6-yl]cyclopropanecarboxamide (100.0 mg, 0.21 mmol) in DCM (2.0 mL) was added TFA (1.0 mL) at room temperature. The resulting mixture was stirred at room temperature for 2 h. The resulting mixture was concentrated under reduced pressure. To the above residue was added NH₃·H₂O (2.0 mL) and ACN (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 10 min; 254 nm) to afford N-[3-[2-methoxy-6-(methylamino)pyridin-3-yl]-1H-pyrrolo[2,3-b]pyridin-6-yl]cyclopropanecarboxamide (28.4 mg, 39%) as a white solid. LCMS (ESI, m/z): [M+H]⁺=338.2. ¹H NMR (300 MHz, DMSO-d₆): δ 11.36 (s, 1H), 10.53 (s, 1H), 7.96 (d, J=8.4 Hz, 1H), 7.85 (d, J=8.7 Hz, 1H), 7.61 (d, J=8.1 Hz, 1H), 7.42 (d, J=2.4 Hz, 1H), 6.39-6.34 (m, 1H), 6.10 (d, J=8.1 Hz, 1H), 3.87 (s, 3H), 2.82 (d, J=4.5 Hz, 3H), 2.06-2.01 (m, 1H), 0.83-0.75 (m, 4H).

### Example S31. Compound 31

### Step 1: Synthesis of tert-butyl N-(tert-butoxycarbonyl)-N-[5-(6-chloro-1-[[2-(trimethylsilyl)ethoxy]methyl]pyrrolo[2,3-b]pyridin-3-yl)-4-methoxypyridin-2-yl]carbamate

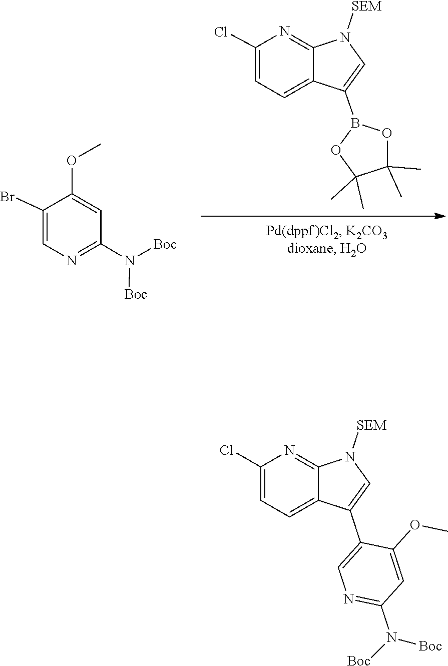 [SMILES: COc1cc(N(C(=O)OC(C)(C)C)C(=O)OC(C)(C)C)ncc1Br ; CC1(C)OB(c2cn(COCC[Si](C)(C)C)c3nc(Cl)ccc23)OC1(C)C ; COc1cc(N(C(=O)OC(C)(C)C)C(=O)OC(C)(C)C)ncc1-c1cn(COCC[Si](C)(C)C)c2nc(Cl)ccc12]

To a solution of tert-butyl N-(5-bromo-4-methoxypyridin-2-yl)-N-(tert-butoxycarbonyl)carbamate (402.5 mg, 1.00 mmol) in dioxane/H₂O (15.0/3.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (340.0 mg, 0.83 mmol), K₂CO₃ (344.8 mg, 2.50 mmol), Pd(dppf)Cl₂ (67.9 mg, 0.08 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford tert-butyl N-(tert-butoxycarbonyl)-N-[5-(6-chloro-1-[[2-(trimethylsilyl)ethoxy]methyl]pyrrolo[2,3-b]pyridin-3-yl)-4-methoxypyridin-2-yl]carbamate (330.0 mg, 66%) as a yellow oil. LCMS (ESI, m/z): [M+H]⁺=605.2.

### Step 2: Synthesis of tert-butyl N-(tert-butoxycarbonyl)-N-[5-(6-cyclopropaneamido-1-[[2-(trimethylsilyl)ethoxy]methyl]pyrrolo[2,3-b]pyridin-3-yl)-4-methoxypyridin-2-yl]carbamate

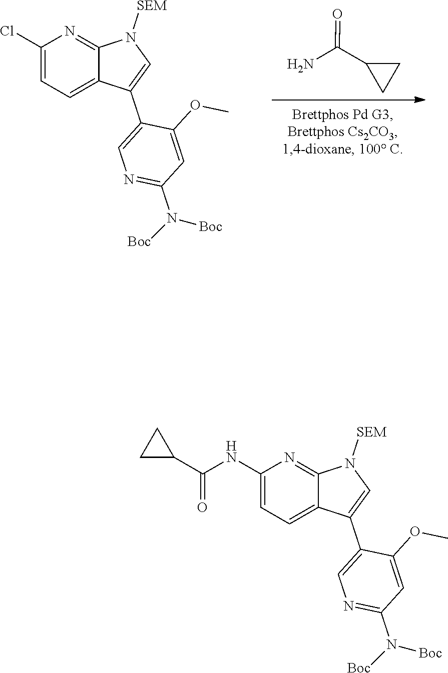 [SMILES: COc1cc(N(C(=O)OC(C)(C)C)C(=O)OC(C)(C)C)ncc1-c1cn(COCC[Si](C)(C)C)c2nc(Cl)ccc12 ; NC(=O)C1CC1 ; COc1cc(N(C(=O)OC(C)(C)C)C(=O)OC(C)(C)C)ncc1-c1cn(COCC[Si](C)(C)C)c2nc(NC(=O)C3CC3)ccc12]

To a solution of tert-butyl N-(tert-butoxycarbonyl)-N-[5-(6-chloro-1-[[2-(trimethylsilyl)ethoxy]methyl]pyrrolo[2,3-b]pyridin-3-yl)-4-methoxypyridin-2-yl]carbamate (282.0 mg, 0.47 mmol) in 1,4-dioxane (15.0 mL) was added cyclopropanecarboxamide (198.3 mg, 2.33 mmol), BrettPhos (50.0 mg, 0.03 mmol), Cs₂CO₃ (455.46 mg, 1.40 mmol) and BrettPhos Pd G3 (42.3 mg, 0.05 mmol) at room temperature under N₂. The resulting mixture was irradiated with microwave radiation at 120° C. for 1.5 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to tert-butyl N-(tert-butoxycarbonyl)-N-[5-(6-cyclopropaneamido-1-[[2-(trimethylsilyl)ethoxy]methyl]pyrrolo[2,3-b]pyridin-3-yl)-4-methoxypyridin-2-yl]carbamate (80.0 mg, 26%) as a yellow oil. LCMS (ESI, m/z): [M+H]⁺=654.3.

### Step 3: Synthesis of N-[3-(6-amino-4-methoxypyridin-3-yl)-1H-pyrrolo[2,3-b]pyridin-6-yl]cyclopropanecarboxamide (Compound 31)

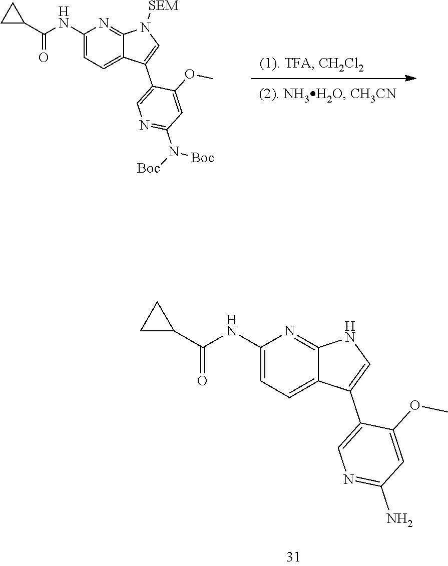 [SMILES: COc1cc(N(C(=O)OC(C)(C)C)C(=O)OC(C)(C)C)ncc1-c1cn(COCC[Si](C)(C)C)c2nc(NC(=O)C3CC3)ccc12 ; COc1cc(N)ncc1-c1c[nH]c2nc(NC(=O)C3CC3)ccc12]

To a solution of tert-butyl N-(tert-butoxycarbonyl)-N-[5-(6-cyclopropaneamido-1-[[2-(trimethylsilyl)ethoxy]methyl]pyrrolo[2,3-b]pyridin-3-yl)-4-methoxypyridin-2-yl]carbamate (80.0 mg, 0.12 mmol) in CH₂Cl₂ (4.0 mL) was added TFA (4.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 14% B to 36% B in 7 min; 254 nm) to afford N-[3-(6-amino-4-methoxypyridin-3-yl)-1H-pyrrolo[2,3-b]pyridin-6-yl]cyclopropanecarboxamide (10.8 mg, 27%) as a white solid. LCMS (ESI, m/z): [M+H]⁺=324.2. ¹H NMR (300 MHz, DMSO-d₆): δ 11.33 (s, 1H), 10.47 (s, 1H), 7.84-7.78 (m, 3H), 7.29 (d, J=2.1 Hz, 1H), 6.09 (s, 1H), 5.78 (s, 2H), 3.70 (s, 3H), 1.96-1.92 (m, 1H), 0.75-0.68 (m, 4H).

### Example S32. Compound 32

### Step 1: Synthesis of tert-butyl (5-bromo-6-methylpyridin-2-yl)carbamate

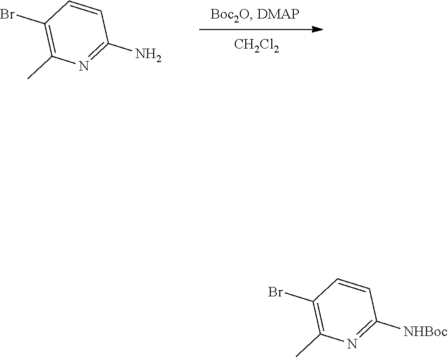

To a solution of 5-bromo-6-methylpyridin-2-amine (5.0 g, 26.73 mmol) in CH₂Cl₂ (100.0 mL) was added Boc₂O (5.8 g, 26.73 mmol) and DMAP (3.3 g, 26.73 mmol) at room temperature. The resulting mixture was stirred at room temperature for 16 h under N₂. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford tert-butyl (5-bromo-6-methylpyridin-2-yl)carbamate (4.0 g, 52%) as a white solid. LCMS (ESI, m/z): [M+H]⁺=287.0.

### Step 2: Synthesis of tert-butyl (6-methyl-5-(4,4,5,5-tetramethyl-1,3,2-dioxaborolan-2-yl)pyridin-2-yl)carbamate

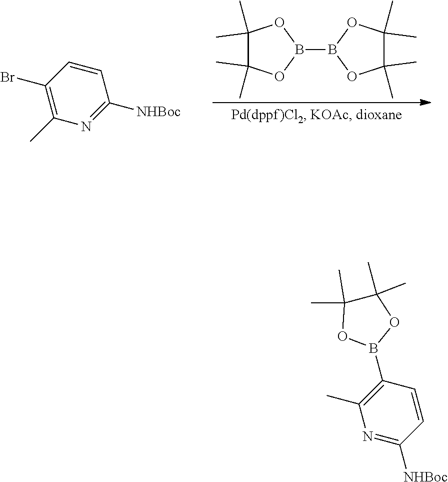

To a solution of tert-butyl (5-bromo-6-methylpyridin-2-yl)carbamate (2.0 g, 6.97 mmol) in dioxane (40.0 mL) was added 4,4,4′,4′,5,5,5′,5′-octamethyl-2,2′-bi(1,3,2-dioxaborolane) (5.3 g, 20.89 mmol), KOAc (2.1 g, 20.89 mmol) and Pd(dppf)Cl₂ (0.5 g, 0.70 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl (6-methyl-5-(4,4,5,5-tetramethyl-1,3,2-dioxaborolan-2-yl)pyridin-2-yl)carbamate (2.0 g, 85%) as a white solid. LCMS (ESI, m/z): [M+H]⁺=335.2.

### Step 3: Synthesis of tert-butyl (5-(6-chloro-1-((2-(trimethylsilyl)ethoxy)methyl)-1H-pyrrolo[2,3-b]pyridin-3-yl)-6-methylpyridin-2-yl)carbamate

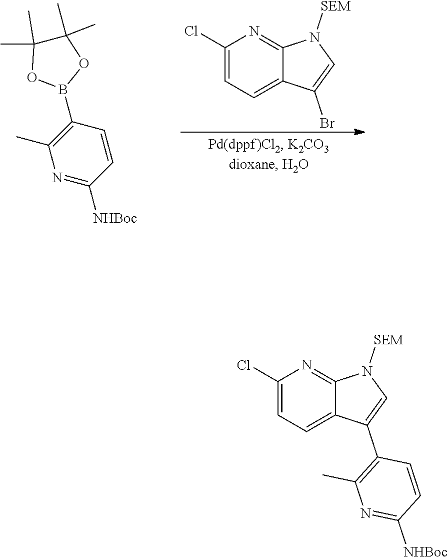 [SMILES: Cc1nc(NC(=O)OC(C)(C)C)ccc1B1OC(C)(C)C(C)(C)O1 ; C[Si](C)(C)CCOCn1cc(Br)c2ccc(Cl)nc21 ; Cc1nc(NC(=O)OC(C)(C)C)ccc1-c1cn(COCC[Si](C)(C)C)c2nc(Cl)ccc12]

To a solution of tert-butyl (6-methyl-5-(4,4,5,5-tetramethyl-1,3,2-dioxaborolan-2-yl)pyridin-2-yl)carbamate (500.0 mg, 1.50 mmol) in dioxane/H₂O (10.0/1.0 mL) was added 3-bromo-6-chloro-1-((2-(trimethylsilyl)ethoxy)methyl)-1H-pyrrolo[2,3-b]pyridine (541.2 mg, 1.50 mmol), K₂CO₃ (620.3 mg, 4.49 mmol) and Pd(dppf)Cl₂ (109.5 mg, 0.15 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford tert-butyl (5-(6-chloro-1-((2-(trimethylsilyl)ethoxy)methyl)-1H-pyrrolo[2,3-b]pyridin-3-yl)-6-methylpyridin-2-yl)carbamate (170.0 mg, 23%) as a yellow solid. LCMS (ESI, m/z): [M+H]⁺=489.2.

### Step 4: Synthesis of tert-butyl (5-(6-(cyclopropanecarboxamido)-1-((2-(trimethylsilyl)ethoxy)methyl)-1H-pyrrolo[2,3-b]pyridin-3-yl)-6-methylpyridin-2-yl)carbamate

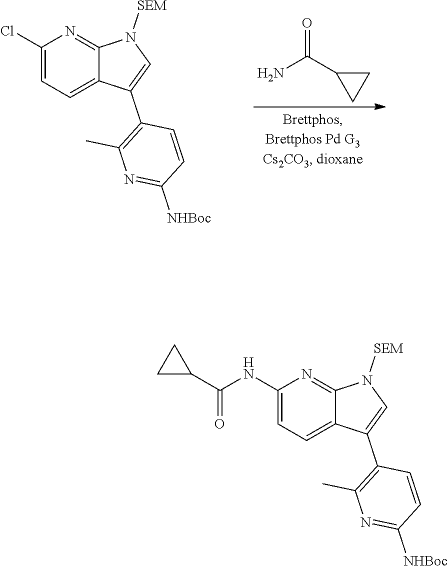 [SMILES: Cc1nc(NC(=O)OC(C)(C)C)ccc1-c1cn(COCC[Si](C)(C)C)c2nc(Cl)ccc12 ; NC(=O)C1CC1 ; Cc1nc(NC(=O)OC(C)(C)C)ccc1-c1cn(COCC[Si](C)(C)C)c2nc(NC(=O)C3CC3)ccc12]

To a solution of tert-butyl (5-(6-chloro-1-((2-(trimethylsilyl)ethoxy)methyl)-1H-pyrrolo[2,3-b]pyridin-3-yl)-6-methylpyridin-2-yl)carbamate (130.0 mg, 0.27 mmol) in dioxane (10.0 mL) was added cyclopropanecarboxamide (135.7 mg, 1.60 mmol), Cs₂CO₃ (259.8 mg, 0.80 mmol), Brettphos (28.5 mg, 0.05 mmol) and BrettPhos Pd G3 (24.1 mg, 0.03 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl (5-(6-(cyclopropanecarboxamido)-1-((2-(trimethylsilyl)ethoxy)methyl)-1H-pyrrolo[2,3-b]pyridin-3-yl)-6-methylpyridin-2-yl)carbamate (130.0 mg, 90%) as a white solid. LCMS (ESI, m/z): [M+H]⁺=538.3.

### Step 5: Synthesis of N-(3-(6-amino-2-methylpyridin-3-yl)-1H-pyrrolo[2,3-b]pyridin-6-yl)cyclopropanecarboxamide (Compound 32)

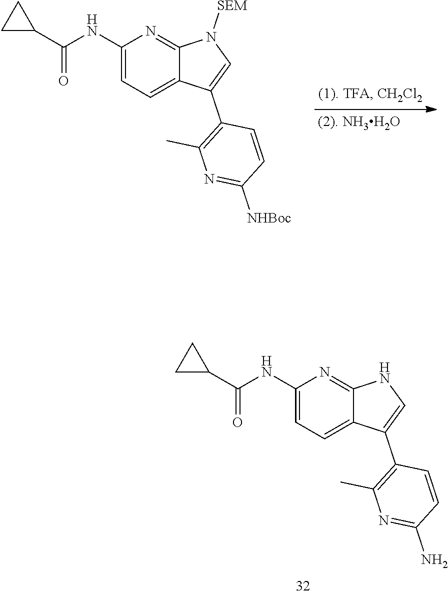 [SMILES: Cc1nc(NC(=O)OC(C)(C)C)ccc1-c1cn(COCC[Si](C)(C)C)c2nc(NC(=O)C3CC3)ccc12 ; Cc1nc(N)ccc1-c1c[nH]c2nc(NC(=O)C3CC3)ccc12]

To a solution of tert-butyl (5-(6-(cyclopropanecarboxamido)-1-((2-(trimethylsilyl)ethoxy)methyl)-1H-pyrrolo[2,3-b]pyridin-3-yl)-6-methylpyridin-2-yl)carbamate (250.0 mg, 0.47 mmol) in CH₂Cl₂ (5.0 mL) was added TFA (5.0 mL) at room temperature. The resulting mixture was stirred at room temperature for 3 h. The resulting mixture was concentrated under reduced pressure. The residue was re-dissolved in CH₃CN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 25 mL/min; Gradient: 6% to 17% in 7 min; 254 nm) to afford N-(3-(6-amino-2-methylpyridin-3-yl)-1H-pyrrolo[2,3-b]pyridin-6-yl)cyclopropanecarboxamide (27.8 mg, 19%) as a white solid. LCMS (ESI, m/z): [M+H]⁺=308.2. ¹H NMR (400 MHz, DMSO-d₆): δ 11.46 (s, 1H), 10.57 (s, 1H), 7.86 (d, J=8.8 Hz, 1H), 7.73 (d, J=8.4 Hz, 1H), 7.36-7.30 (m, 2H), 6.38 (d, J=8.4 Hz, 1H), 5.86 (s, 2H), 2.27 (s, 3H), 2.07-2.01 (m, 1H), 0.84-0.81 (m, 4H).

### Example S33. Compound 33

### Step 1: Synthesis of 5-bromo-N,6-dimethylpyridin-2-amine

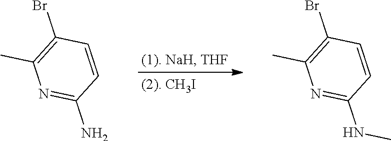

To a solution of 5-bromo-6-methylpyridin-2-amine (10.0 g, 53.46 mmol) in THF (100.0 mL) was added NaH (1.9 g, 60%) at 0° C. under N₂. The resulting mixture was stirred at 0° C. for 1 h under N₂. Then CH₃I (7.6 g, 53.46 mmol) was added dropwise to the mixture at 0° C. under N₂. The resulting mixture was stirred at 0° C. for another 1 h. After the reaction was completed, the reaction was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 5-bromo-N,6-dimethylpyridin-2-amine (3.3 g, 30%) as a yellow solid. LCMS (ESI, m/z): [M+H]⁺=201.0.

### Step 2: Synthesis of N,6-dimethyl-5-(4,4,5,5-tetramethyl-1,3,2-dioxaborolan-2-yl)pyridin-2-amine

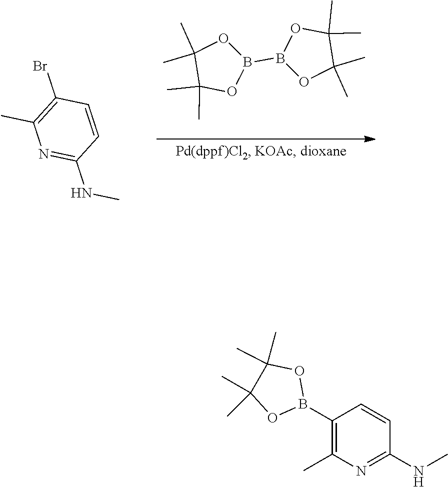

To a solution of 5-bromo-N,6-dimethylpyridin-2-amine (2.0 g, 9.95 mmol) in dioxane (40.0 mL) was added 4,4,4′,4′,5,5,5′,5′-octamethyl-2,2′-bi(1,3,2-dioxaborolane) (7.6 g, 29.84 mmol), KOAc (8.9 g, 29.84 mmol) and Pd(dppf)Cl₂ (0.7 g, 1.00 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford N,6-dimethyl-5-(4,4,5,5-tetramethyl-1,3,2-dioxaborolan-2-yl)pyridin-2-amine (750.0 mg, 30%) as a brown oil. LCMS (ESI, m/z): [M+H]⁺=249.2.

### Step 3: Synthesis of 5-(6-chloro-1-((2-(trimethylsilyl)ethoxy)methyl)-1H-pyrrolo[2,3-b]pyridin-3-yl)-N,6-dimethylpyridin-2-amine

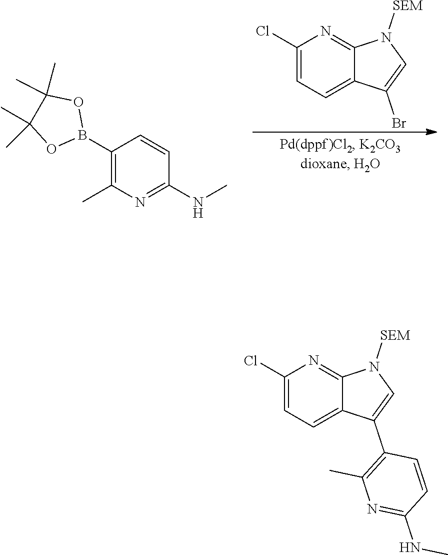

To a solution of N,6-dimethyl-5-(4,4,5,5-tetramethyl-1,3,2-dioxaborolan-2-yl)pyridin-2-amine (500.0 mg, 2.02 mmol) in dioxane/H₂O (10.0/1.0 mL) was added 3-bromo-6-chloro-1-((2-(trimethylsilyl)ethoxy)methyl)-1H-pyrrolo[2,3-b]pyridine (728.9 mg, 2.02 mmol), K₂CO₃ (835.5 mg, 6.05 mmol) and Pd(dppf)Cl₂ (147.4 mg, 0.20 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 5-(6-chloro-1-((2-(trimethylsilyl)ethoxy)methyl)-1H-pyrrolo[2,3-b]pyridin-3-yl)-N,6-dimethylpyridin-2-amine (250.0 mg, 30%) as a yellow oil. LCMS (ESI, m/z): [M+H]⁺=403.2.

### Step 4: Synthesis of N-(3-(2-methyl-6-(methylamino)pyridin-3-yl)-1-((2-(trimethylsilyl)ethoxy)methyl)-1H-pyrrolo[2,3-b]pyridin-6-yl)cyclopropanecarboxamide

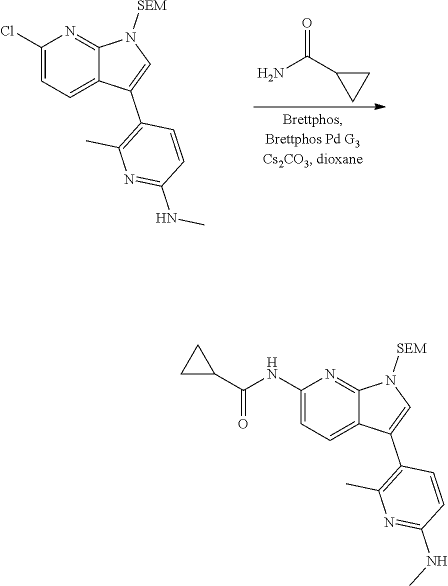 [SMILES: CNc1ccc(-c2cn(COCC[Si](C)(C)C)c3nc(Cl)ccc23)c(C)n1 ; NC(=O)C1CC1 ; CNc1ccc(-c2cn(COCC[Si](C)(C)C)c3nc(NC(=O)C4CC4)ccc23)c(C)n1]

To a solution of 5-(6-chloro-1-((2-(trimethylsilyl)ethoxy)methyl)-1H-pyrrolo[2,3-b]pyridin-3-yl)-N,6-dimethylpyridin-2-amine (200.0 mg, 0.50 mmol) in dioxane (10.0 mL) was added cyclopropanecarboxamide (253.4 mg, 2.98 mmol), Cs₂CO₃ (485.1 mg, 1.49 mmol), Brettphos (53.3 mg, 0.10 mmol) and BrettPhos Pd G3 (45.0 mg, 0.05 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-(3-(2-methyl-6-(methylamino)pyridin-3-yl)-1-((2-(trimethylsilyl)ethoxy)methyl)-1H-pyrrolo[2,3-b]pyridin-6-yl)cyclopropanecarboxamide (150.0 mg, 66%) as a white solid. LCMS (ESI, m/z): [M+H]⁺=452.2.

### Step 5: Synthesis of N-(3-(2-methyl-6-(methylamino)pyridin-3-yl)-1H-pyrrolo[2,3-b]pyridin-6-yl)cyclopropanecarboxamide (Compound 33)

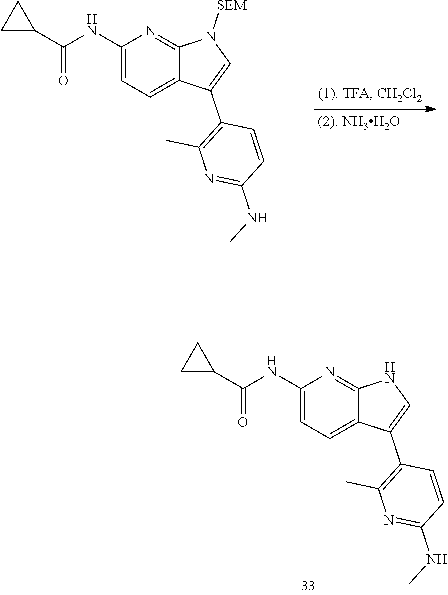 [SMILES: CNc1ccc(-c2cn(COCC[Si](C)(C)C)c3nc(NC(=O)C4CC4)ccc23)c(C)n1 ; CNc1ccc(-c2c[nH]c3nc(NC(=O)C4CC4)ccc23)c(C)n1]

To a solution of N-(3-(2-methyl-6-(methylamino)pyridin-3-yl)-1-((2-(trimethylsilyl)ethoxy)methyl)-1H-pyrrolo[2,3-b]pyridin-6-yl)cyclopropanecarboxamide (130.0 mg, 0.29 mmol) in CH₂Cl₂ (5.0 mL) was added TFA (5.0 mL) at room temperature. The resulting mixture was stirred at room temperature for 3 h. The resulting mixture was concentrated under reduced pressure. The residue was re-dissolved in CH₃CN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 24% to 44% in 7 min; 254 nm) to afford N-(3-(2-methyl-6-(methylamino)pyridin-3-yl)-1H-pyrrolo[2,3-b]pyridin-6-yl)cyclopropanecarboxamide (44.1 mg, 47%) as a white solid. LCMS (ESI, m/z): [M+H]⁺=322.2. ¹H NMR (300 MHz, DMSO-d₆): δ 11.46 (d, J=1.5 Hz, 1H), 10.57 (s, 1H), 7.87 (d, J=8.7 Hz, 1H), 7.73 (d, J=8.4 Hz, 1H), 7.36 (d, J=8.4 Hz, 1H), 7.29 (d, J=2.4 Hz, 1H), 6.36-6.31 (m, 2H), 2.79 (d, J=4.8 Hz, 3H), 2.30 (s, 3H), 2.08-2.00 (m, 1H), 0.85-0.75 (m, 4H).

### Example S34. Compound 34

### Step 1: Synthesis of 6-[(tert-butoxycarbonyl)amino]-4-methylpyridin-3-ylboronic acid

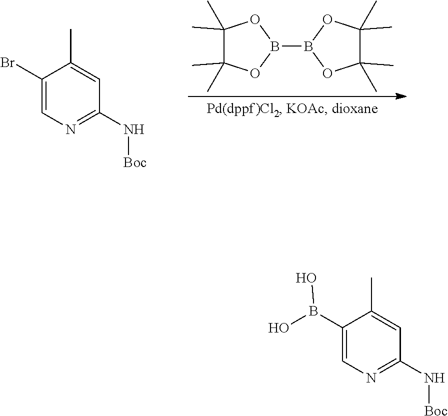

To a solution of tert-butyl N-(5-bromo-4-methylpyridin-2-yl)carbamate (500.0 mg, 1.71 mmo) in dioxane (10.0 mL) was added 4,4,4′,4′,5,5,5′,5′-octamethyl-2,2′-bi(1,3,2-dioxaborolane) (1326.4 mg, 5.24 mmol), Pd(dppf)Cl₂ (254.8 mg, 0.38 mmol) and KOAc (512.6 mg, 5.24 mmol) at room temperature under N₂. The resulting mixture was stirred at 80° C. for 16 h. After the reaction was completed, the resulting mixture was concentrated under reduced pressure. The residue was purified by flash column chromatography with petroleum ether/ethyl acetate (1/2, v/v) to afford 6-[(tert-butoxycarbonyl)amino]-4-methylpyridin-3-ylboronic acid (400.0 mg, 69%) as a yellow oil. LCMS (ESI, m/z): [M+H]⁺=253.1.

### Step 2: Synthesis of tert-butyl N-[5-(6-chloro-1-[[2-(trimethylsilyl)ethoxy]methyl]pyrrolo[2,3-b]pyridin-3-yl)-4-methylpyridin-2-yl]carbamate

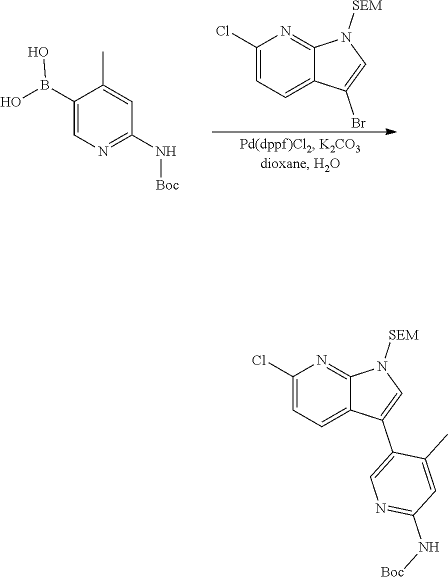 [SMILES: Cc1cc(NC(=O)OC(C)(C)C)ncc1B(O)O ; C[Si](C)(C)CCOCn1cc(Br)c2ccc(Cl)nc21 ; Cc1cc(NC(=O)OC(C)(C)C)ncc1-c1cn(COCC[Si](C)(C)C)c2nc(Cl)ccc12]

To a solution of 6-[(tert-butoxycarbonyl)amino]-4-methylpyridin-3-ylboronic acid (300.0 mg, 1.19 mmol) in dioxane/H₂O (10.0 mL/1.0 mL) was added 3-bromo-6-chloro-1-[[2-(trimethylsilyl)ethoxy]methyl]pyrrolo[2,3-b]pyridine (344.4 mg, 0.95 mmol), Pd(dppf)Cl₂ (87.0 mg, 0.19 mmol) and K₂CO₃ (493.4 mg, 3.57 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[5-(6-chloro-1-[[2-(trimethylsilyl)ethoxy]methyl]pyrrolo[2,3-b]pyridin-3-yl)-4-methylpyridin-2-yl]carbamate (130.0 mg, 22%) as a yellow oil. LCMS (ESI, m/z): [M+H]⁺=489.2.

### Step 3: Synthesis of tert-butyl N-[5-(6-cyclopropaneamido-1-[[2-(trimethylsilyl)ethoxy]methyl]pyrrolo[2,3-b]pyridin-3-yl)-4-methylpyridin-2-yl]carbamate

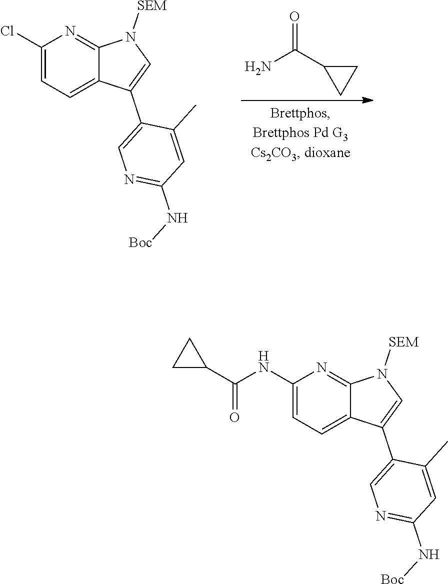 [SMILES: Cc1cc(NC(=O)OC(C)(C)C)ncc1-c1cn(COCC[Si](C)(C)C)c2nc(Cl)ccc12 ; NC(=O)C1CC1 ; Cc1cc(NC(=O)OC(C)(C)C)ncc1-c1cn(COCC[Si](C)(C)C)c2nc(NC(=O)C3CC3)ccc12]

To a solution of tert-butyl N-[5-(6-chloro-1-[[2-(trimethylsilyl)ethoxy]methyl]pyrrolo[2,3-b]pyridin-3-yl)-4-methylpyridin-2-yl]carbamate (110.0 mg, 0.25 mmol) in dioxane (10.0 mL) was added cyclopropanecarboxamide (57.4 mg, 0.65 mmol), BrettPhos Pd G3 (20.9 mg, 0.02 mmol), BrettPhos (24.4 mg, 0.05 mmol) and Cs₂CO₃ (219.8 mg, 0.67 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[5-(6-cyclopropaneamido-1-[[2-(trimethylsilyl)ethoxy]methyl]pyrrolo[2,3-b]pyridin-3-yl)-4-methylpyridin-2-yl]carbamate (78.0 mg, 64%) as a white oil. LCMS (ESI, m/z): [M+H]⁺=538.3.

### Step 4: Synthesis of N-[3-(6-amino-4-methylpyridin-3-yl)-1H-pyrrolo[2,3-b]pyridin-6-yl]cyclopropanecarboxamide (Compound 34)

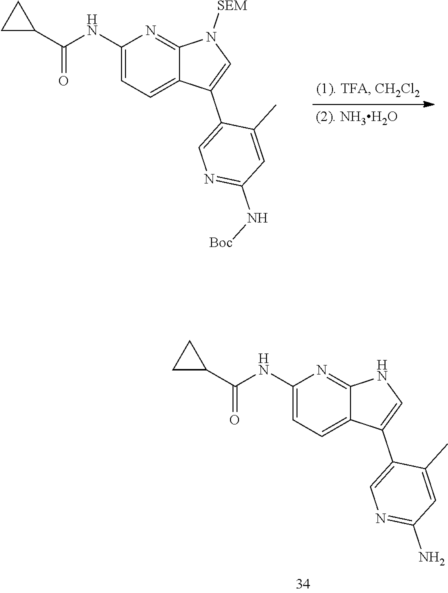 [SMILES: Cc1cc(NC(=O)OC(C)(C)C)ncc1-c1cn(COCC[Si](C)(C)C)c2nc(NC(=O)C3CC3)ccc12 ; Cc1cc(N)ncc1-c1c[nH]c2nc(NC(=O)C3CC3)ccc12]

To a solution of tert-butyl N-[5-(6-cyclopropaneamido-1-[[2-(trimethylsilyl)ethoxy]methyl]pyrrolo[2,3-b]pyridin-3-yl)-4-methylpyridin-2-yl]carbamate (78.0 mg, 0.14 mmol) in CH₂Cl₂ (5.0 mL) was added TFA (5.0 mL) at room temperature. The resulting mixture was stirred at room temperature for 2 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was concentrated under reduced pressure. The residue washed with CH₂Cl₂ and then filtered. The solid was collected and dried to afford N-[3-(6-amino-4-methylpyridin-3-yl)-1H-pyrrolo[2,3-b]pyridin-6-yl]cyclopropanecarboxamide (10.0 mg, 22%) as a white solid. LCMS (ESI, m/z): [M+H]⁺=308.1. ¹H NMR (300 MHz, DMSO-d₆): δ 11.82 (s, 1H), 10.72 (s, 1H), 8.02-7.90 (m, 3H), 7.64-7.53 (m, 3H), 6.93 (s, 1H), 2.37 (s, 3H), 2.13-2.07 (m, 1H), 0.93-0.85 (m, 4H).

### Example S35. Compound 35

### Step 1: Synthesis of tert-butyl N-(5-bromo-4-methylpyridin-2-yl)carbamate

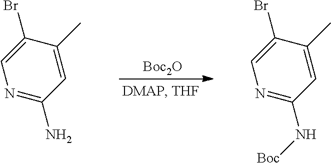

To a solution of 5-bromo-4-methylpyridin-2-amine (5.0 g, 26.72 mmol) in THF (100.0 mL) was added di-tert-butyl dicarbonate (5.8 g, 26.72 mmol) and DMAP (3.7 g, 26.72 mmol) at room temperature under N₂. The resulting mixture was stirred at room temperature for 16 h. After the reaction was completed, the resulting mixture was concentrated under reduced pressure. The residue was purified by flash column chromatography with petroleum ether/ethyl acetate (6/1, v/v) to afford tert-butyl N-(5-bromo-4-methylpyridin-2-yl)carbamate (2.7 g, 35%) as a white solid. LCMS (ESI, m/z): [M+H]⁺=287.0

### Step 2: Synthesis of tert-butyl N-(5-bromo-4-methylpyridin-2-yl)-N-methylcarbamate

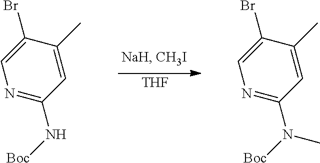 [SMILES: Cc1cc(NC(=O)OC(C)(C)C)ncc1Br ; Cc1cc(N(C)C(=O)OC(C)(C)C)ncc1Br ; "THF"]

To a solution of tert-butyl N-(5-bromo-4-methylpyridin-2-yl)carbamate (700.0 mg, 2.48 mmol) in THF (10.0 mL) was added NaH (117.0 mg, 60%) at 0° C. under N₂. The resulting mixture was stirred at 0° C. for 1 h. Then CH₃I (692.0 mg, 4.88 mmol) was added to the mixture. The resulting mixture was stirred at 0° C. for another 1 h. After the reaction was completed, the resulting mixture was quenched with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/1, v/v) to afford tert-butyl N-(5-bromo-4-methylpyridin-2-yl)-N-methylcarbamate (600.0 mg, 95%) as a yellow oil. LCMS (ESI, m/z): [M+H]⁺=301.0.

### Step 3: Synthesis of tert-butyl methyl(4-methyl-5-(4,4,5,5-tetramethyl-1,3,2-dioxaborolan-2-yl)pyridin-2-yl)carbamate

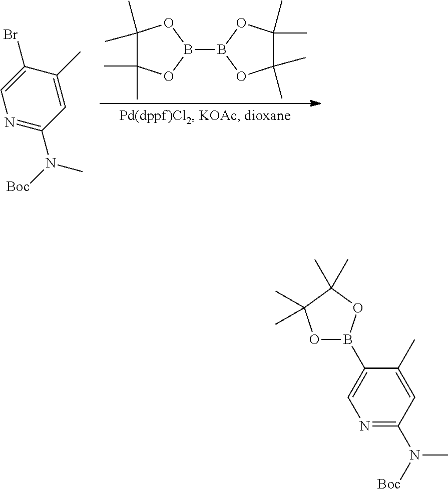

To a solution of tert-butyl N-(5-bromo-4-methylpyridin-2-yl)-N-methylcarbamate (600.0 mg, 1.92 mmol) in dioxane (10.0 mL, 510.75 mmol) was added 4,4,4′,4′,5,5,5′,5′-octamethyl-2,2′-bi(1,3,2-dioxaborolane) (1.5 g, 5.98 mmol), Pd(dppf)Cl₂ (145.7 mg, 0.19 mmol) and KOAc (586.5 mg, 5.97 mmol) at room temperature under N₂. The resulting mixture was stirred at 80° C. for 16 h. After the reaction was completed, the resulting mixture was concentrated under reduced pressure. The residue was purified by flash column chromatography with petroleum ether/ethyl acetate (7/1, v/v) to afford tert-butyl methyl(4-methyl-5-(4,4,5,5-tetramethyl-1,3,2-dioxaborolan-2-yl)pyridin-2-yl)carbamate (500.0 mg, 72%) as a yellow oil. LCMS (ESI, m/z): [M+H]⁺=349.2.

### Step 4: Synthesis of tert-butyl N-[5-(6-chloro-1-[[2-(trimethylsilyl)ethoxy]methyl]pyrrolo[2,3-b]pyridin-3-yl)-4-methylpyridin-2-yl]-N-methylcarbamate

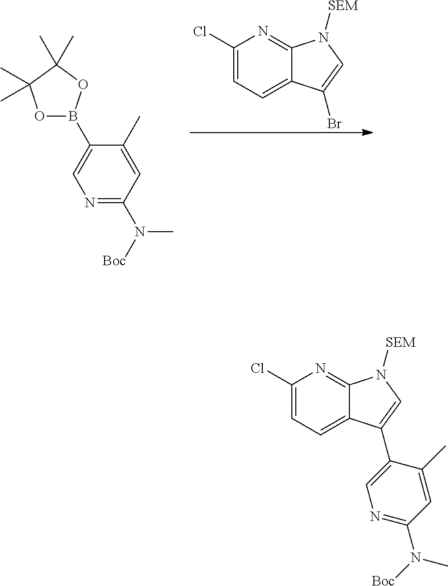

To a solution of tert-butyl methyl(4-methyl-5-(4,4,5,5-tetramethyl-1,3,2-dioxaborolan-2-yl)pyridin-2-yl)carbamate (400.0 mg, 1.50 mmol) in dioxane/H₂O (10.0/1.0 mL) was added 3-bromo-6-chloro-1-[[2-(trimethylsilyl)ethoxy]methyl]pyrrolo[2,3-b]pyridine (1.1 g, 3.06 mmol), Pd(dppf)Cl₂ (109.9 mg, 0.15 mmol) and K₂CO₃ (623.5 mg, 4.50 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[5-(6-chloro-1-[[2-(trimethylsilyl)ethoxy]methyl]pyrrolo[2,3-b]pyridin-3-yl)-4-methylpyridin-2-yl]-N-methylcarbamate (400.0 mg, 53%) as a yellow oil. LCMS (ESI, m/z): [M+H]⁺=503.2.

### Step 5: Synthesis of tert-butyl N-[5-(6-cyclopropaneamido-1-[[2-(trimethylsilyl)ethoxy]methyl]pyrrolo[2,3-b]pyridin-3-yl)-4-methylpyridin-2-yl]-N-methylcarbamate

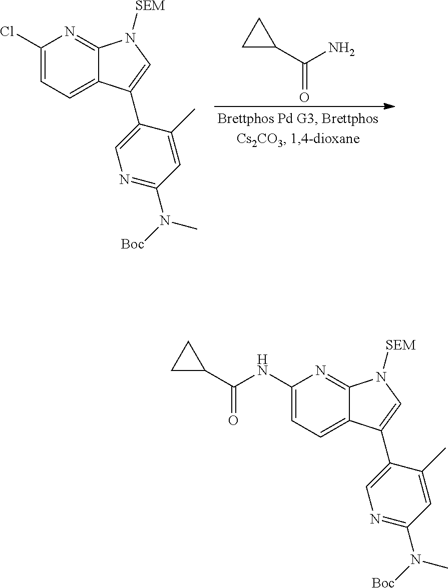 [SMILES: Cc1cc(N(C)C(=O)OC(C)(C)C)ncc1-c1cn(COCC[Si](C)(C)C)c2nc(Cl)ccc12 ; NC(=O)C1CC1 ; Cc1cc(N(C)C(=O)OC(C)(C)C)ncc1-c1cn(COCC[Si](C)(C)C)c2nc(NC(=O)C3CC3)ccc12]

To a solution of tert-butyl N-[5-(6-chloro-1-[[2-(trimethylsilyl)ethoxy]methyl]pyrrolo[2,3-b]pyridin-3-yl)-4-methylpyridin-2-yl]-N-methylcarbamate (380.0 mg, 0.75 mmol) in dioxane (10.0 mL) was added cyclopropanecarboxamide (192.8 mg, 2.26 mmol), BrettPhos Pd G3 (68.7 mg, 0.07 mmol), BrettPhos (81.0 mg, 0.15 mmol) and Cs₂CO₃ (738.6 mg, 2.26 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[5-(6-cyclopropaneamido-1-[[2-(trimethylsilyl)ethoxy]methyl]pyrrolo[2,3-b]pyridin-3-yl)-4-methylpyridin-2-yl]-N-methylcarbamate (300.0 mg, 72%) as a yellow oil. LCMS (ESI, m/z): [M+H]⁺=552.3.

### Step 6: Synthesis of N-[3-[4-methyl-6-(methylamino)pyridin-3-yl]-1H-pyrrolo[2,3-b]pyridin-6-yl]cyclopropanecarboxamide (Compound 35)

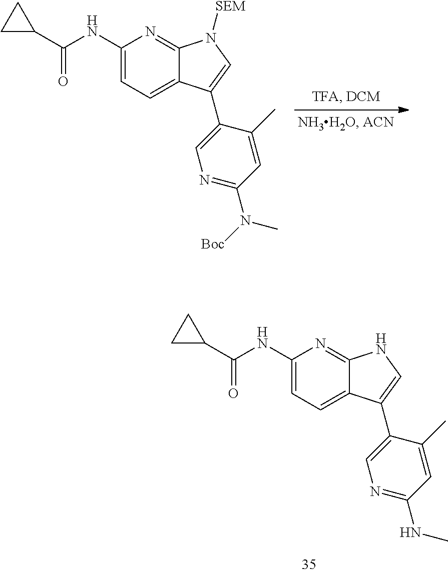 [SMILES: Cc1cc(N(C)C(=O)OC(C)(C)C)ncc1-c1cn(COCC[Si](C)(C)C)c2nc(NC(=O)C3CC3)ccc12 ; CNc1cc(C)c(-c2c[nH]c3nc(NC(=O)C4CC4)ccc23)cn1]

To a solution of tert-butyl N-[5-(6-cyclopropaneamido-1-[[2-(trimethylsilyl)ethoxy]methyl]pyrrolo[2,3-b]pyridin-3-yl)-4-methylpyridin-2-yl]-N-methylcarbamate (150.0 mg, 0.27 mmol) in DCM (5.0 mL) was added TFA (5.0 mL) at room temperature. The resulting mixture was stirred at room temperature for 2 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 16% B to 46% B in 10 min; 254 nm) to afford N-[3-[4-methyl-6-(methylamino)pyridin-3-yl]-1H-pyrrolo[2,3-b]pyridin-6-yl]cyclopropanecarboxamide (17.4 mg, 20%) as a white solid. LCMS (ESI, m/z): [M+H]⁺=322.2. ¹H NMR (300 MHz, DMSO-d₆): δ 11.47 (s, 1H), 10.56 (s, 1H), 7.92 (s, 1H), 7.86 (d, J=8.4 Hz, 1H), 7.73 (d, J=8.7 Hz, 1H), 7.30 (d, J=2.1 Hz, 1H), 6.39 (s, 1H), 6.32-6.28 (m, 1H), 2.79 (d, J=4.8 Hz, 3H), 2.14 (s, 3H), 2.05-2.01 (m, 1H), 0.85-0.77 (m, 4H).

### Example S36. Compound 36

### Step 1: Synthesis of 3-(6-chloro-1-[[2-(trimethylsilyl)ethoxy]methyl]pyrrolo[2,3-b]pyridin-3-yl)pyridin-2-amine

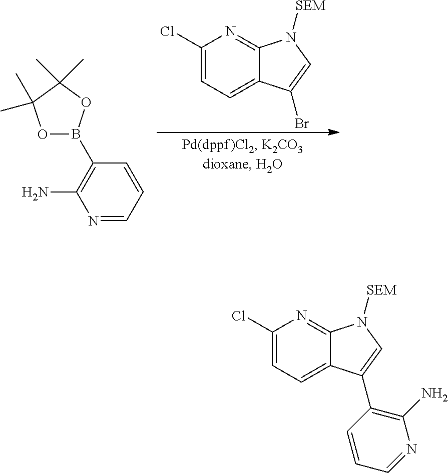 [SMILES: CC1(C)OB(c2cccnc2N)OC1(C)C ; C[Si](C)(C)CCOCn1cc(Br)c2ccc(Cl)nc21 ; C[Si](C)(C)CCOCn1cc(-c2cccnc2N)c2ccc(Cl)nc21]

To a solution of 3-(4,4,5,5-tetramethyl-1,3,2-dioxaborolan-2-yl)pyridin-2-amine (500.0 mg, 2.27 mmol) in dioxane/H₂O (5.0/0.5 mL) was added 3-bromo-6-chloro-1-[[2-(trimethylsilyl)ethoxy]methyl]pyrrolo[2,3-b]pyridine (986.2 mg, 2.72 mmol), Pd(dppf)Cl₂ (332.4 mg, 0.45 mmol) and K₂CO₃ (941.9 mg, 6.81 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (62/38, v/v) to afford 3-(6-chloro-1-[[2-(trimethylsilyl)ethoxy]methyl]pyrrolo[2,3-b]pyridin-3-yl)pyridin-2-amine (150.0 mg, 44%) as a yellow oil. LCMS (ESI, m/z): [M+H]⁺=375.1.

### Step 2: Synthesis of tert-butyl N-[3-(6-chloro-1-[[2-(trimethylsilyl)ethoxy]methyl]pyrrolo[2,3-b]pyridin-3-yl)pyridin-2-yl]carbamate

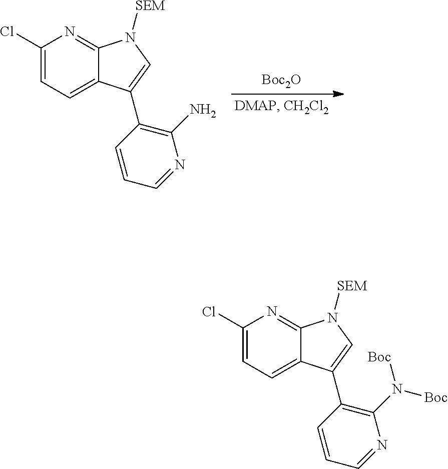 [SMILES: "Boc2O" ; C[Si](C)(C)CCOCn1cc(-c2cccnc2N)c2ccc(Cl)nc21 ; CC(C)(C)OC(=O)N(C(=O)OC(C)(C)C)c1ncccc1-c1cn(COCC[Si](C)(C)C)c2nc(Cl)ccc12]

To a solution of 3-(6-chloro-1-[[2-(trimethylsilyl)ethoxy]methyl]pyrrolo[2,3-b]pyridin-3-yl)pyridin-2-amine (330.0 mg, 0.88 mmol) in DCM (5.0 mL) was added di-tert-butyl dicarbonate (768.3 mg, 3.52 mmol), TEA (267.1 mg, 2.64 mmol) and DMAP (21.5 mg, 0.17 mmol) at room temperature. The resulting mixture was stirred at room temperature for 5 h. After the reaction was completed, the resulting mixture was concentrated under reduced pressure. The residue was purified by flash column chromatography with petroleum ether/ethyl acetate (50/50, v/v) to afford tert-butyl N-[3-(6-chloro-1-[[2-(trimethylsilyl)ethoxy]methyl]pyrrolo[2,3-b]pyridin-3-yl)pyridin-2-yl]carbamate (460.0 mg, 72%) as a yellow oil. LCMS (ESI, m/z): [M+H]⁺=575.2.

### Step 3: Synthesis of tert-butyl N-(tert-butoxycarbonyl)-N-[3-(6-cyclopropaneamido-1-[[2-(trimethylsilyl)ethoxy]methyl]pyrrolo[2,3-b]pyridin-3-yl)pyridin-2-yl]carbamate

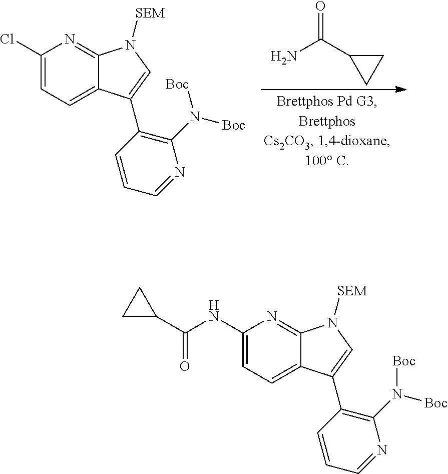 [SMILES: CC(C)(C)OC(=O)N(C(=O)OC(C)(C)C)c1ncccc1-c1cn(COCC[Si](C)(C)C)c2nc(Cl)ccc12 ; NC(=O)C1CC1 ; CC(C)(C)OC(=O)N(C(=O)OC(C)(C)C)c1ncccc1-c1cn(COCC[Si](C)(C)C)c2nc(NC(=O)C3CC3)ccc12]

To a solution of tert-butyl N-(tert-butoxycarbonyl)-N-[3-(6-chloro-1-[[2-(trimethylsilyl)ethoxy]methyl]pyrrolo[2,3-b]pyridin-3-yl)pyridin-2-yl]carbamate (440.0 mg, 0.76 mmol) in dioxane (5.0 mL) was added cyclopropanecarboxamide (325.5 mg, 3.82 mmol), BrettPhos (164 mg, 0.30 mmol), BrettPhos Pd G3 (138.6 mg, 0.15 mmol) and Cs₂CO₃ (747.7 mg, 2.29 mmol) at room temperature under N₂. The resulting mixture was stirred at 100° C. for 5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (76/24, v/v) to afford tert-butyl N-(tert-butoxycarbonyl)-N-[3-(6-cyclopropaneamido-1-[[2-(trimethylsilyl)ethoxy]methyl]pyrrolo[2,3-b]pyridin-3-yl)pyridin-2-yl]carbamate (360.0 mg, 75%) as a yellow oil. LCMS (ESI, m/z): [M+H]⁺=624.3.

### Step 4: Synthesis of N-[3-(2-aminopyridin-3-yl)-1H-pyrrolo[2,3-b]pyridin-6-yl]cyclopropanecarboxamide (Compound 36)

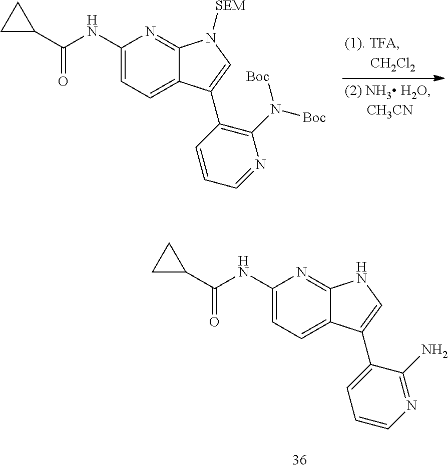 [SMILES: CC(C)(C)OC(=O)N(C(=O)OC(C)(C)C)c1ncccc1-c1cn(COCC[Si](C)(C)C)c2nc(NC(=O)C3CC3)ccc12 ; Nc1ncccc1-c1c[nH]c2nc(NC(=O)C3CC3)ccc12]

To a solution of tert-butyl N-(tert-butoxycarbonyl)-N-[3-(6-cyclopropaneamido-1-[[2-(trimethylsilyl)ethoxy]methyl]pyrrolo[2,3-b]pyridin-3-yl)pyridin-2-yl]carbamate (310.0 mg, 0.49 mmol) in DCM (2.0 mL) was added TFA (2.0 mL). The mixture was stirred at room temperature for 3 h. The resulting mixture was concentrated under reduced pressure. The residue was re-dissolved in ACN (2.0 mL) and NH₃·H₂O (2.0 mL). The mixture was stirred at room temperature for another 5 h. After the reaction was completed, the resulting mixture was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 45% B to 75% B in 7 min; 254 nm) to afford N-[3-(2-aminopyridin-3-yl)-1H-pyrrolo[2,3-b]pyridin-6-yl]cyclopropanecarboxamide (48.4 mg, 33%) as a white solid. LCMS (ESI, m/z): [M+H]⁺=294.1. ¹H NMR (300 MHz, DMSO-d₆): δ 10.62 (s, 1H), 7.94-7.83 (m, 3H), 7.53-7.47 (m, 2H), 6.68-6.64 (m, 1H), 6.04 (s, 1H), 5.56 (s, 2H), 2.08-2.00 (m, 1H), 0.86-0.81 (m, 4H).

### Example S37. Compound 37

### Step 1: Synthesis of tert-butyl N-[3-(6-chloro-1-[[2-(trimethylsilyl)ethoxy]methyl]pyrrolo[2,3-b]pyridin-3-yl)pyridin-2-yl]-N-methylcarbamate

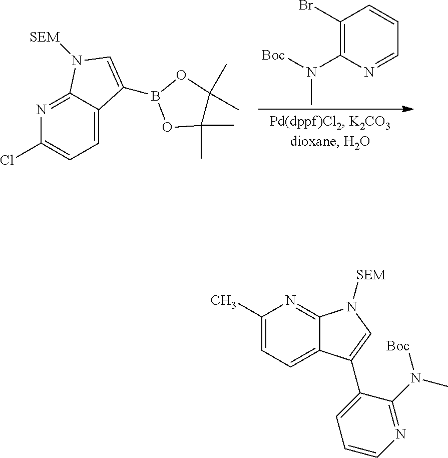

To a solution of 6-chloro-3-(4,4,5,5-tetramethyl-1,3,2-dioxaborolan-2-yl)-1-[[2-(trimethylsilyl)ethoxy]methyl]pyrrolo[2,3-b]pyridine (220.0 mg, 0.53 mmol) in 1,4-dioxane/H₂O (4.0/0.4 mL) was added tert-butyl N-(3-bromopyridin-2-yl)-N-methylcarbamate (154.5 mg, 0.53 mmol), Pd(dppf)Cl₂ (78.7 mg, 0.10 mmol) and K₂CO₃ (223.1 mg, 1.61 mmol) at room temperature under N₂, The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford tert-butyl N-[3-(6-chloro-1-[[2-(trimethylsilyl)ethoxy]methyl]pyrrolo[2,3-b]pyridin-3-yl)pyridin-2-yl]-N-methylcarbamate (140.0 mg, 53%) as a yellow oil. LCMS (ESI, m/z): [M+H]⁺=489.2

### Step 2: Synthesis of tert-butyl N-[3-(6-cyclopropaneamido-1-[[2-(trimethylsilyl)ethoxy]methyl]pyrrolo[2,3-b]pyridin-3-yl)pyridin-2-yl]-N-methylcarbamate

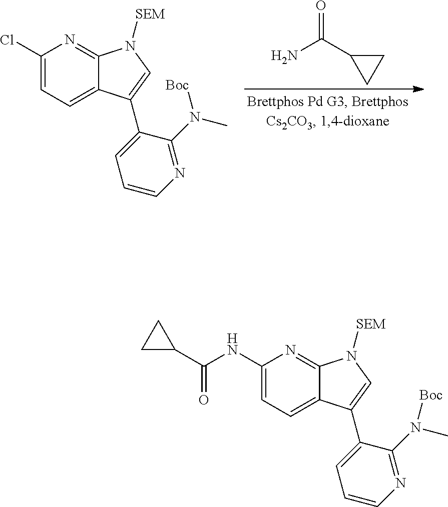 [SMILES: CN(C(=O)OC(C)(C)C)c1ncccc1-c1cn(COCC[Si](C)(C)C)c2nc(Cl)ccc12 ; NC(=O)C1CC1 ; CN(C(=O)OC(C)(C)C)c1ncccc1-c1cn(COCC[Si](C)(C)C)c2nc(NC(=O)C3CC3)ccc12]

To a solution of tert-butyl N-[3-(6-chloro-1-[[2-(trimethylsilyl)ethoxy]methyl]pyrrolo[2,3-b]pyridin-3-yl)pyridin-2-yl]-N-methylcarbamate (140.0 mg, 0.28 mmol) in dioxane (4.0 mL) was added cyclopropanecarboxamide (121.8 mg, 1.43 mmol), BrettPhos (61.5 mg, 0.11 mmol), BrettPhos Pd G3 (51.9 mg, 0.11 mmol) and Cs₂CO₃ (279.8 mg, 0.56 mmol) at room temperature under N₂, The resulting mixture was stirred at 100° C. for 5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford tert-butyl N-[3-(6-cyclopropaneamido-1-[[2-(trimethylsilyl)ethoxy]methyl]pyrrolo[2,3-b]pyridin-3-yl)pyridin-2-yl]-N-methylcarbamate (120.0 mg, 77%) as a yellow solid. LCMS (ESI, m/z): [M+H]⁺=538.3.

### Step 3: Synthesis of N-[3-[2-(methylamino)pyridin-3-yl]-1H-pyrrolo[2,3-b]pyridin-6-yl]cyclopropanecarboxamide (Compound 37)

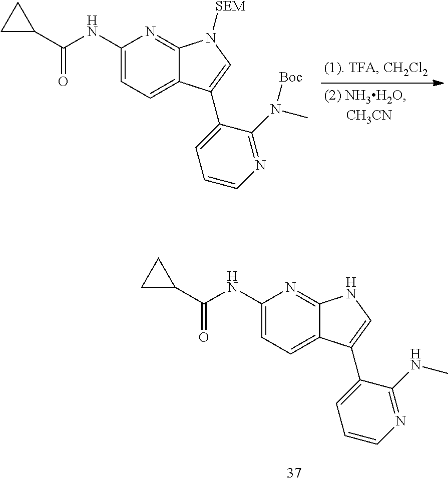 [SMILES: CN(C(=O)OC(C)(C)C)c1ncccc1-c1cn(COCC[Si](C)(C)C)c2nc(NC(=O)C3CC3)ccc12 ; CNc1ncccc1-c1c[nH]c2nc(NC(=O)C3CC3)ccc12]

To a solution of tert-butyl N-[3-(6-cyclopropaneamido-1-[[2-(trimethylsilyl)ethoxy]methyl]pyrrolo[2,3-b]pyridin-3-yl)pyridin-2-yl]-N-methylcarbamate (200.0 mg, 0.37 mmol) in DCM (2.0 mL) was added TFA (0.5 mL). The mixture was stirred at room temperature for 3 h. The resulting mixture was concentrated under reduced pressure. The residue was dissolved in NH₃·H₂O (0.5 mL) and CH₃CN (2.0 mL). The mixture was stirred at room temperature for another 6 h. After the reaction was completed, the resulting mixture was concentrated under reduced pressure. The residue was purified by flash column chromatography with CH₂Cl₂/CH₃OH (75/25, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% B to 57% B in 7 min; 254 nm) to afford N-[3-[2-(methylamino)pyridin-3-yl]-1H-pyrrolo[2,3-b]pyridin-6-yl]cyclopropanecarboxamide (10.7 mg, 98%) as a white solid. LCMS (ESI, m/z): [M+H]⁺=308.1. ¹H NMR (300 MHz, DMSO-d₆): δ 11.65 (s, 1H), 10.59 (s, 1H), 8.02-8.00 (m, 1H), 7.90-7.84 (m, 1H), 7.77 (d, J=8.7 Hz, 1H), 7.50 (s, 1H), 7.41-7.38 (m, 1H), 6.62-6.58 (m, 1H), 5.76-5.73 (m, 1H), 2.79 (d, J=4.5 Hz, 3H), 2.05-1.99 (m, 1H), 0.92-0.75 (m, 4H).

### Example S38. Compound 38

### Step 1: Synthesis of tert-butyl (6-bromopyridin-2-yl)carbamate

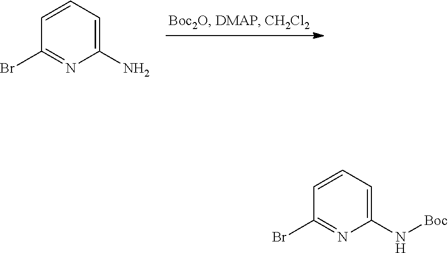 [SMILES: Nc1cccc(Br)n1 ; CC(C)(C)OC(=O)Nc1cccc(Br)n1]

To a solution of 6-bromopyridin-2-amine (5.0 g, 28.90 mmol) in CH₂Cl₂ (100.0 mL) was added Boc₂O (6.3 g, 28.90 mmol) and DMAP (3.5 g, 28.90 mmol) at room temperature. The resulting mixture was stirred at room temperature for 16 h. After the reaction was completed, the resulting mixture was washed with brine, dried over anhydrous sodium sulfate and filtered. The filtrate was concentrated under reduced pressure. The residue was purified by flash column chromatography with petroleum ether/ethyl acetate (88/12, v/v) to afford tert-butyl (6-bromopyridin-2-yl)carbamate (4.3 g, 54%) as a white solid. LCMS (ESI, m/z): [M+H]⁺=273.0.

### Step 2: Synthesis of 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine

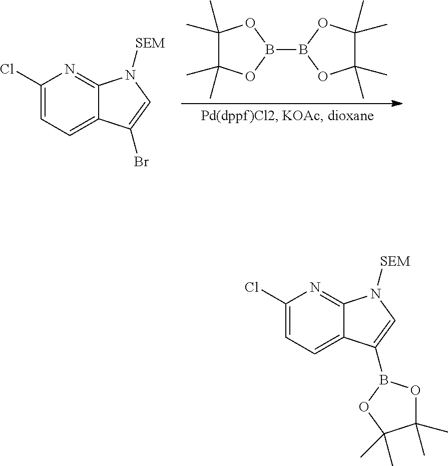

To a solution of 3-bromo-6-chloro-1-((2-(trimethylsilyl)ethoxy)methyl)-1H-pyrrolo[2,3-b]pyridine (1.0 g, 1.55 mmol) in 1,4-dioxane (15.0 mL) was added 4,4,4′,4′,5,5,5′,5′-octamethyl-2,2′-bi(1,3,2-dioxaborolane) (1.2 g, 4.65 mmol), KOAc (460.0 mg, 4.65 mmol) and Pd(dppf)Cl₂ (113.3 mg, 0.16 mmol) at room temperature under N₂. The resulting mixture was stirred at 80° C. for 16 h. After the reaction was completed, the resulting mixture was concentrated under reduced pressure. The residue was purified by flash column chromatography with petroleum ether/ethyl acetate (92/8, v/v) to afford 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (330.0 mg, 52%) as a colorless oil. LCMS (ESI, m/z): [M+H]⁺=409.2.

### Step 3: Synthesis of tert-butyl (6-(6-chloro-1-((2-(trimethylsilyl)ethoxy)methyl)-1H-pyrrolo[2,3-b]pyridin-3-yl)pyridin-2-yl)carbamate

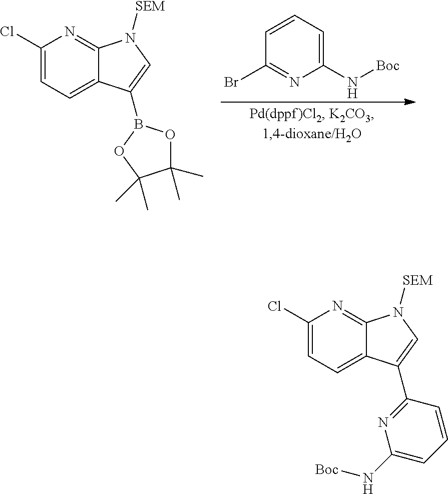 [SMILES: CC1(C)OB(c2cn(COCC[Si](C)(C)C)c3nc(Cl)ccc23)OC1(C)C ; CC(C)(C)OC(=O)Nc1cccc(Br)n1 ; CC(C)(C)OC(=O)Nc1cccc(-c2cn(COCC[Si](C)(C)C)c3nc(Cl)ccc23)n1]

To a solution of 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (300.0 mg, 0.73 mmol) in 1,4-dioxane/H₂O (10.0/2.0 mL) was added tert-butyl (6-bromopyridin-2-yl)carbamate (200.4 mg, 0.73 mmol), K₂CO₃ (304.3 mg, 2.20 mmol) and Pd(dppf)Cl₂ (53.7 mg, 0.07 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (92/8, v/v) to afford tert-butyl (6-(6-chloro-1-((2-(trimethylsilyl)ethoxy)methyl)-1H-pyrrolo[2,3-b]pyridin-3-yl)pyridin-2-yl)carbamate (150.0 mg, 43%) as a colorless oil. LCMS (ESI, m/z): [M+H]⁺=475.2.

### Step 4: Synthesis of tert-butyl (6-(6-(cyclopropanecarboxamido)-1-((2-(trimethylsilyl)ethoxy)methyl)-1H-pyrrolo[2,3-b]pyridin-3-yl)pyridin-2-yl)carbamate

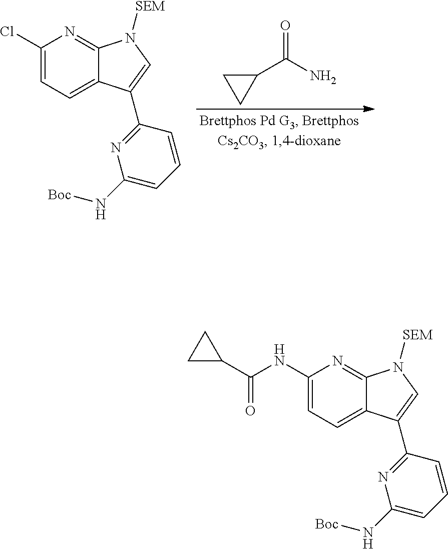 [SMILES: CC(C)(C)OC(=O)Nc1cccc(-c2cn(COCC[Si](C)(C)C)c3nc(Cl)ccc23)n1 ; NC(=O)C1CC1 ; CC(C)(C)OC(=O)Nc1cccc(-c2cn(COCC[Si](C)(C)C)c3nc(NC(=O)C4CC4)ccc23)n1]

To a solution of tert-butyl (6-(6-chloro-1-((2-(trimethylsilyl)ethoxy)methyl)-1H-pyrrolo[2,3-b]pyridin-3-yl)pyridin-2-yl)carbamate (130.0 mg, 0.27 mmol) in 1,4-dioxane (10.0 mL) was added cyclopropanecarboxamide (116.5 mg, 1.37 mmol), Cs₂CO₃ (267.5 mg, 0.82 mmol), BrettPhos (29.4 mg, 0.06 mmol) and BrettPhos Pd G3 (24.8 mg, 0.03 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford tert-butyl (6-(6-(cyclopropanecarboxamido)-1-((2-(trimethylsilyl)ethoxy)methyl)-1H-pyrrolo[2,3-b]pyridin-3-yl)pyridin-2-yl)carbamate (140.0 mg, 97%) as a yellow solid. LCMS (ESI, m/z): [M+H]⁺=524.3.

### Step 5: Synthesis of N-(3-(6-aminopyridin-2-yl)-1H-pyrrolo[2,3-b]pyridin-6-yl)cyclopropanecarboxamide (Compound 38)

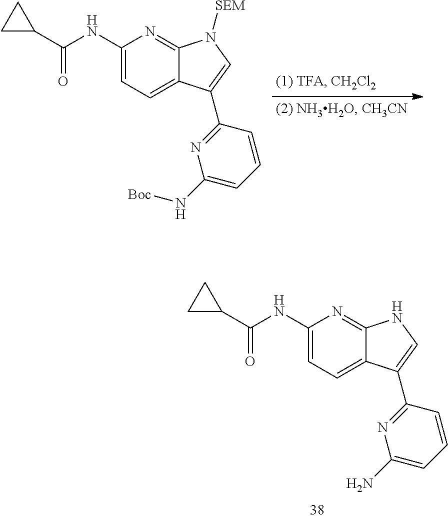 [SMILES: CC(C)(C)OC(=O)Nc1cccc(-c2cn(COCC[Si](C)(C)C)c3nc(NC(=O)C4CC4)ccc23)n1 ; Nc1cccc(-c2c[nH]c3nc(NC(=O)C4CC4)ccc23)n1]

To a solution of tert-butyl (6-(6-(cyclopropanecarboxamido)-1-((2-(trimethylsilyl)ethoxy)methyl)-1H-pyrrolo[2,3-b]pyridin-3-yl)pyridin-2-yl)carbamate (120.0 mg, 0.23 mmol) in CH₂Cl₂ (6.0 mL) was added TFA (6.0 mL) at room temperature. The resulting mixture was stirred at room temperature for 4 h. After the reaction was completed, the resulting mixture was concentrated under reduced pressure. The residue was re-dissolved in CH₃CN (6.0 mL) was added NH₃·H₂O (6.0 mL). The resulting mixture was stirred at room temperature for 2 h. After the reaction was completed, the resulting mixture was concentrated under reduced pressure. The solid was washed with CH₂Cl₂ and H₂O to afford N-(3-(6-aminopyridin-2-yl)-1H-pyrrolo[2,3-b]pyridin-6-yl)cyclopropanecarboxamide (31.1 mg, 46%) as a white solid. LCMS (ESI, m/z): [M+H]⁺=294.1. ¹H NMR (300 MHz, DMSO-d₆): δ 11.60 (s, 1H), 10.58 (s, 1H), 8.77 (d, J=8.7 Hz, 1H), 7.95-7.91 (m, 2H), 7.39-7.34 (m, 1H), 6.99 (d, J=7.5 Hz, 1H), 6.25 (d, J=8.1 Hz, 1H), 5.86 (s, 2H), 2.08-2.00 (m, 1H), 0.83-0.79 (m, 4H).

### Example S39. Compound 39

### Step 1: Synthesis of tert-butyl (6-bromopyridin-2-yl)(methyl)carbamate

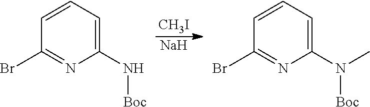

To a solution of tert-butyl (6-bromopyridin-2-yl)carbamate (1.0 g, 3.67 mmol) in DMF (30.0 mL) was added NaH (439.3 mg, 60%) at 0° C. under N₂. The resulting mixture was stirred at 0° C. for 1 h. Then CH₃I (2.6 g, 18.31 mmol) was added dropwise to the mixture at 0° C. The resulting mixture was stirred at 0° C. for another 1 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (91/9, v/v) to afford tert-butyl (6-bromopyridin-2-yl)(methyl)carbamate (1.0 g, 95%) as a colorless oil. LCMS (ESI, m/z): [M+H]⁺=287.0.

### Step 2: Synthesis of tert-butyl (6-(6-chloro-1-((2-(trimethylsilyl)ethoxy)methyl)-1H-pyrrolo[2,3-b]pyridin-3-yl)pyridin-2-yl)(methyl)carbamate

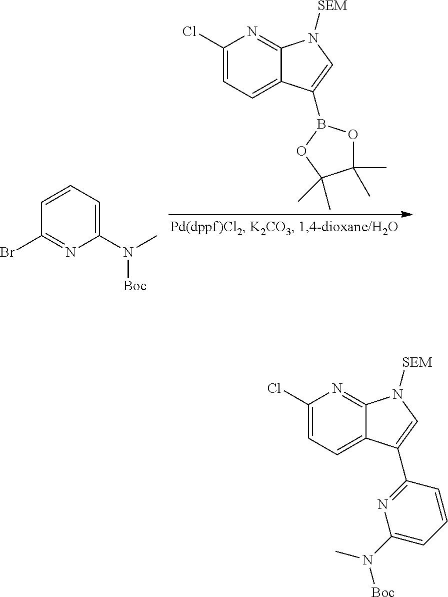 [SMILES: CN(C(=O)OC(C)(C)C)c1cccc(Br)n1 ; CC1(C)OB(c2cn(COCC[Si](C)(C)C)c3nc(Cl)ccc23)OC1(C)C ; CN(C(=O)OC(C)(C)C)c1cccc(-c2cn(COCC[Si](C)(C)C)c3nc(Cl)ccc23)n1]

To a solution of 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (440.0 mg, 1.07 mmol) in 1,4-dioxane/H₂O (10.0/2.0 mL) was added tert-butyl (6-bromopyridin-2-yl)(methyl)carbamate (309.0 mg, 1.07 mmol), K₂CO₃ (448.1 mg, 3.21 mmol) and Pd(dppf)Cl₂ (78.6 mg, 0.11 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (92/8, v/v) to afford tert-butyl (6-(6-chloro-1-((2-(trimethylsilyl)ethoxy)methyl)-1H-pyrrolo[2,3-b]pyridin-3-yl)pyridin-2-yl)(methyl)carbamate (270.0 mg, 53%) as a colorless oil. LCMS (ESI, m/z): [M+H]⁺=489.2.

### Step 3: Synthesis of tert-butyl (6-(6-(cyclopropanecarboxamido)-1-((2-(trimethylsilyl)ethoxy)methyl)-1H-pyrrolo[2,3-b]pyridin-3-yl)pyridin-2-yl)(methyl)carbamate

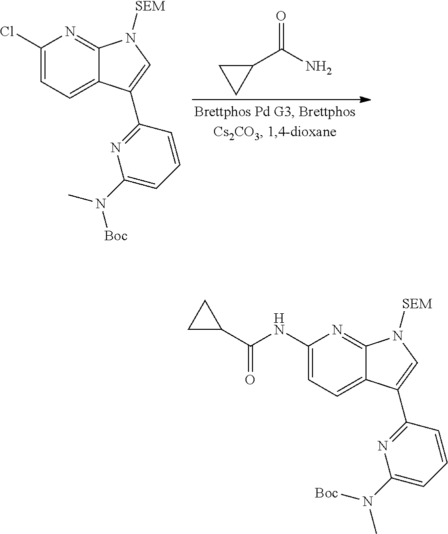 [SMILES: CN(C(=O)OC(C)(C)C)c1cccc(-c2cn(COCC[Si](C)(C)C)c3nc(Cl)ccc23)n1 ; NC(=O)C1CC1 ; CN(C(=O)OC(C)(C)C)c1cccc(-c2cn(COCC[Si](C)(C)C)c3nc(NC(=O)C4CC4)ccc23)n1]

To a solution of tert-butyl (6-(6-chloro-1-((2-(trimethylsilyl)ethoxy)methyl)-1H-pyrrolo[2,3-b]pyridin-3-yl)pyridin-2-yl)(methyl)carbamate (250.0 mg, 0.51 mmol) in 1,4-dioxane (10.0 mL) was added cyclopropanecarboxamide (217.5 mg, 2.56 mmol), Cs₂CO₃ (499.6 mg, 1.53 mmol), BrettPhos (54.9 mg, 0.10 mmol) and BrettPhos Pd G3 (46.3 mg, 0.05 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford tert-butyl (6-(6-(cyclopropanecarboxamido)-1-((2-(trimethylsilyl)ethoxy)methyl)-1H-pyrrolo[2,3-b]pyridin-3-yl)pyridin-2-yl)(methyl)carbamate (240.0 mg, 87%) as a light yellow solid. LCMS (ESI, m/z): [M+H]⁺=538.3.

### Step 4: Synthesis of N-(3-(6-(methylamino)pyridin-2-yl)-1H-pyrrolo[2,3-b]pyridin-6-yl)cyclopropanecarboxamide (Compound 39)

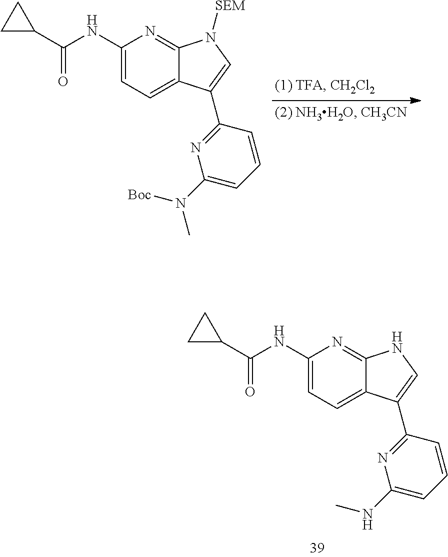 [SMILES: CN(C(=O)OC(C)(C)C)c1cccc(-c2cn(COCC[Si](C)(C)C)c3nc(NC(=O)C4CC4)ccc23)n1 ; CNc1cccc(-c2c[nH]c3nc(NC(=O)C4CC4)ccc23)n1]

To a solution of tert-butyl (6-(6-(cyclopropanecarboxamido)-1-((2-(trimethylsilyl)ethoxy)methyl)-1H-pyrrolo[2,3-b]pyridin-3-yl)pyridin-2-yl)(methyl)carbamate (200.0 mg, 0.37 mmol) in CH₂Cl₂ (6.0 mL) was added TFA (6.0 mL) at room temperature. The resulting mixture was stirred at room temperature for 4 h. The resulting mixture was concentrated under reduced pressure. The residue was re-dissolved in CH₃CN (6.0 mL) was added NH₃·H₂O (6.0 mL). The resulting mixture was stirred at room temperature for another 2 h. After the reaction was completed, the resulting mixture was concentrated under reduced pressure. The solid was washed with CH₂Cl₂ and H₂O. The solid was collected and dried to afford N-(3-(6-(methylamino)pyridin-2-yl)-1H-pyrrolo[2,3-b]pyridin-6-yl)cyclopropanecarboxamide (47.0 mg, 41%) as a white solid. LCMS (ESI, m/z): [M+H]⁺=308.2. ¹H NMR (300 MHz, DMSO-d₆) δ 11.61 (s, 1H), 10.58 (s, 1H), 8.77 (d, J=8.7 Hz, 1H), 7.94-7.92 (m, 2H), 7.40-7.35 (m, 1H), 6.99 (d, J=7.2 Hz, 1H), 6.40-6.39 (m, 1H), 6.24 (d, J=8.1 Hz, 1H), 2.89 (d, J=4.2 Hz, 3H), 2.08-2.00 (m, 1H), 0.83-0.79 (m, 4H).

### Example S40. Compound 40

### Step 1: Synthesis of 5-(6-chloro-1-[[2-(trimethylsilyl)ethoxy]methyl]pyrrolo[2,3-b]pyridin-3-yl)pyridin-2-amine

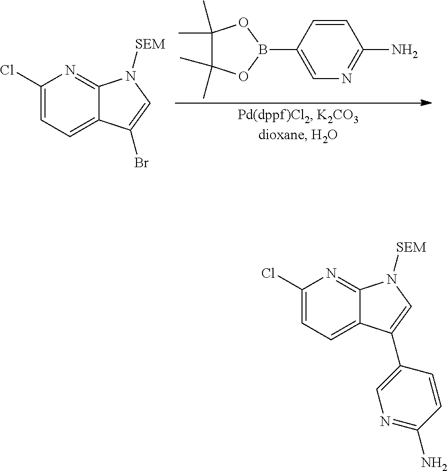

To a solution of 3-bromo-6-chloro-1-[[2-(trimethylsilyl)ethoxy]methyl]pyrrolo[2,3-b]pyridine (1.0 g, 2.76 mmol) in 1,4-dioxane/H₂O (5.0/1.0 mL) was added 5-(4,4,5,5-tetramethyl-1,3,2-dioxaborolan-2-yl)pyridin-2-amine (0.7 g, 3.31 mmol), K₂CO₃ (1.2 g, 8.29 mmol) and Pd(dppf)Cl₂ (0.5 g, 0.55 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (93/7, v/v) to afford 5-(6-chloro-1-[[2-(trimethylsilyl)ethoxy]methyl]pyrrolo[2,3-b]pyridin-3-yl)pyridin-2-amine (300.0 mg, 29%) as a white solid. LCMS (ESI, m/z): [M+H]⁺=375.1.

### Step 2: Synthesis of N-[3-(6-aminopyridin-3-yl)-1-[[2-(trimethylsilyl)ethoxy]methyl]pyrrolo[2,3-b]pyridin-6-yl]cyclopropanecarboxamide

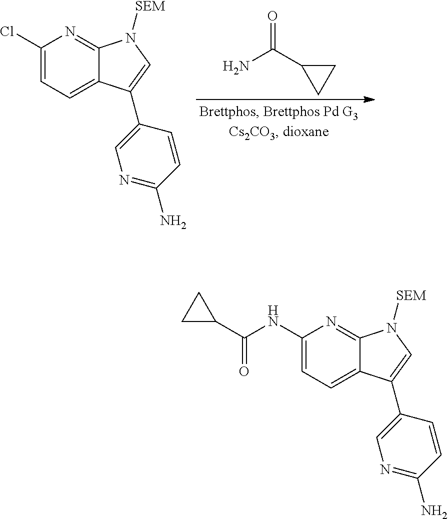 [SMILES: C[Si](C)(C)CCOCn1cc(-c2ccc(N)nc2)c2ccc(Cl)nc21 ; NC(=O)C1CC1 ; C[Si](C)(C)CCOCn1cc(-c2ccc(N)nc2)c2ccc(NC(=O)C3CC3)nc21]

To a solution of 5-(6-chloro-1-[[2-(trimethylsilyl)ethoxy]methyl]pyrrolo[2,3-b]pyridin-3-yl)pyridin-2-amine (300.0 mg, 0.85 mmol) in dioxane (3.0 mL) was added cyclopropanecarboxamide (363.2 mg, 4.27 mmol), BrettPhos (183.3 mg, 0.34 mmol), Cs₂CO₃ (834.2 mg, 2.56 mmol) and BrettPhos Pd G3 (154.7 mg, 0.17 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by silica gel column chromatography with petroleum ether/ethyl acetate (40/60, v/v) to afford N-[3-(6-aminopyridin-3-yl)-1-[[2-(trimethylsilyl)ethoxy]methyl]pyrrolo[2,3-b]pyridin-6-yl]cyclopropanecarboxamide (38.0 mg, 10%) as a white solid. LCMS (ESI, m/z): [M+H]⁺=424.2.

### Step 3: Synthesis of N-[3-(6-aminopyridin-3-yl)-1H-pyrrolo[2,3-b]pyridin-6-yl]cyclopropanecarboxamide (Compound 40)

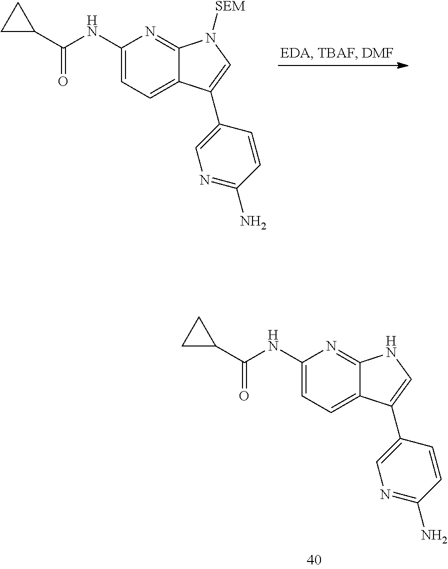 [SMILES: C[Si](C)(C)CCOCn1cc(-c2ccc(N)nc2)c2ccc(NC(=O)C3CC3)nc21 ; Nc1ccc(-c2c[nH]c3nc(NC(=O)C4CC4)ccc23)cn1]

To a solution of N-[3-(6-aminopyridin-3-yl)-1-[[2-(trimethylsilyl)ethoxy]methyl]pyrrolo[2,3-b]pyridin-6-yl]cyclopropanecarboxamide (38.0 mg, 0.09 mmol) in DMF (2.0 mL) was added ethylenediamine (2.0 mL) and TBAF (2.0 mL) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: Xselect CSH OBD Column 30×150 mm, 5 um; Mobile Phase A: Water (0.1% FA), Mobile Phase B: ACN; Flow rate: 60 mL/min; Gradient: 27% B to 49% B in 9 min; 254 nm to afford N-[3-(6-aminopyridin-3-yl)-1H-pyrrolo[2,3-b]pyridin-6-yl]cyclopropanecarboxamide (2.3 mg, 9%) as a white solid. LCMS (ESI, m/z): [M+H]⁺=294.2. ¹H NMR (400 MHz, DMSO-d₆): δ 11.64 (s, 1H), 10.61 (s, 1H), 8.24 (s, 1H), 8.18-8.13 (m, 1H), 8.03 (d, J=8.4 Hz, 1H), 7.93 (d, J=8.4 Hz, 1H), 7.67 (s, 1H), 7.09-6.68 (m, 3H), 2.07-1.99 (m, 1H), 0.82-0.78 (m, 4H).

### Example S41. Compound 41

### Step 1: Synthesis of cis-(1S,2R)-2-formyl-N-[3-(2-methoxyphenyl)-1-[[2-(trimethylsilyl)ethoxy]methyl]pyrrolo[2,3-b]pyridin-6-yl]cyclopropane-1-carboxamide

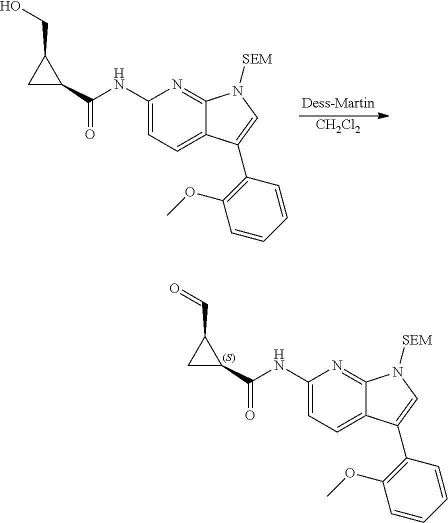

To a solution of cis-2-(hydroxymethyl)-N-[3-(2-methoxyphenyl)-1-[[2-(trimethylsilyl)ethoxy]methyl]pyrrolo[2,3-b]pyridin-6-yl]cyclopropane-1-carboxamide (290.0 mg, 0.62 mmol) in DCM (10.0 mL) was added Dess-Martin (394.5 mg, 0.93 mmol). The resulting mixture was stirred at room temperature for 1 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford cis-2-formyl-N-[3-(2-methoxyphenyl)-1-[[2-(trimethylsilyl)ethoxy]methyl]pyrrolo[2,3-b]pyridin-6-yl]cyclopropane-1-carboxamide (280.0 mg, crude) as a yellow solid. LCMS (ESI, m/z): [M+H]⁺=466.2.

### Step 2: Synthesis of cis-N-[3-(2-methoxyphenyl)-1-[[2-(trimethylsilyl)ethoxy]methyl]pyrrolo[2,3-b]pyridin-6-yl]-2-[(4-methylpiperazin-1-yl)methyl]cyclopropane-1-carboxamide

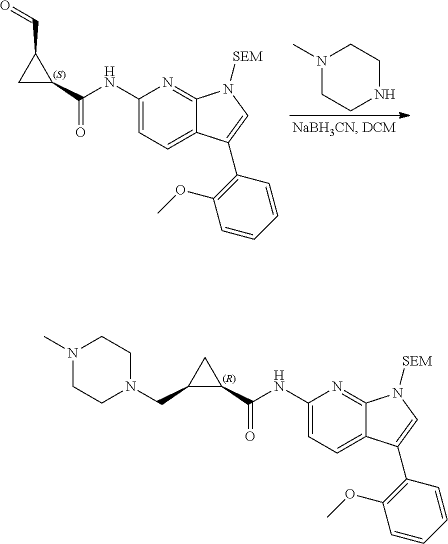

To a solution of cis-2-formyl-N-[3-(2-methoxyphenyl)-1-[[2-(trimethylsilyl)ethoxy]methyl]pyrrolo[2,3-b]pyridin-6-yl]cyclopropane-1-carboxamide (300.0 mg, crude) in CH₂Cl₂ (10.0 mL) was added 1-methylpiperazine (129.1 mg, 1.29 mmol) and NaBH₃CN (81.0 mg, 1.29 mmol). The resulting mixture was stirred at room temperature for 1 h. After the reaction was completed, the resulting mixture was concentrated under vacuum. The residue was purified by reverse phase flash chromatography with CH₃OH/H₂O (60/40, v/v) to afford cis-N-[3-(2-methoxyphenyl)-1-[[2-(trimethylsilyl)ethoxy]methyl]pyrrolo[2,3-b]pyridin-6-yl]-2-[(4-methylpiperazin-1-yl)methyl]cyclopropane-1-carboxamide (280.0 mg, 79%) as a yellow solid. LCMS (ESI, m/z): [M+H]⁺=550.3.

### Step 3: Synthesis of cis-N-[3-(2-methoxyphenyl)-1H-pyrrolo[2,3-b]pyridin-6-yl]-2-[(4-methylpiperazin-1-yl)methyl]cyclopropane-1-carboxamide (Compound 41)

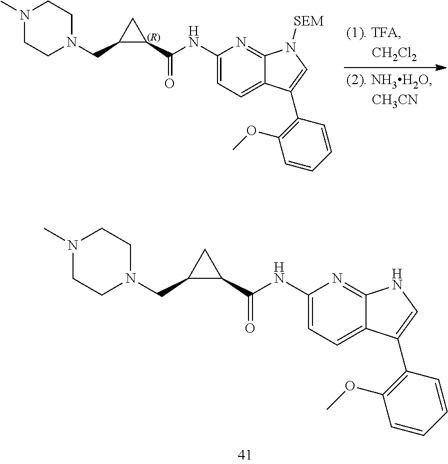

To a solution of cis-N-[3-(2-methoxyphenyl)-1-[[2-(trimethylsilyl)ethoxy]methyl]pyrrolo[2,3-b]pyridin-6-yl]-2-[(4-methylpiperazin-1-yl)methyl]cyclopropane-1-carboxamide (280.0 mg, 0.51 mmol) in CH₂Cl₂ (3.0 mL) was added TFA (3.0 mL). The mixture was stirred at room temperature for 2 h. The mixture was concentrated under vacuum. The residue was dissolved in CH₃CN (3.0 mL) and NH₃·H₂O (3.0 mL). The resulting mixture was stirred at room temperature for another 2 h. After the reaction was completed, the resulting mixture was concentrated under vacuum. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 10 min; 254 nm) to afford cis-N-[3-(2-methoxyphenyl)-1H-pyrrolo[2,3-b]pyridin-6-yl]-2-[(4-methylpiperazin-1-yl)methyl]cyclopropane-1-carboxamide (40.7 mg, 19%) as a white solid. LCMS (ESI, m/z): [M+H]⁺=420.2. ¹H NMR (300 MHz, DMSO-d₆): δ 11.53 (s, 1H), 10.51 (s, 1H), 7.97 (d, J=8.7 Hz, 1H), 7.88 (d, J=8.7 Hz, 1H), 7.56-7.52 (m, 2H), 7.30-7.25 (m, 1H), 7.11 (d, J=7.5 Hz, 1H), 7.05-7.00 (m, 1H), 3.82 (s, 3H), 2.60-2.52 (m, 1H), 2.49-2.40 (m, 5H), 2.28-2.20 (m, 4H), 2.15-2.09 (m, 4H), 1.35-1.30 (m, 1H), 1.02-0.98 (m, 1H), 0.90-0.87 (m, 1H).

### Example S42. Compound 42

### Step 1: Synthesis of N-[3-(2-methoxyphenyl)-1-[[2-(trimethylsilyl)ethoxy]methyl]pyrrolo[2,3-b]pyridin-6-yl]-1,1-diphenylmethanimine

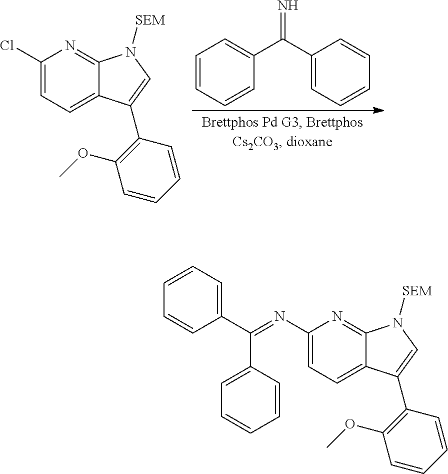 [SMILES: COc1ccccc1-c1cn(COCC[Si](C)(C)C)c2nc(Cl)ccc12 ; N=C(c1ccccc1)c1ccccc1 ; COc1ccccc1-c1cn(COCC[Si](C)(C)C)c2nc(N=C(c3ccccc3)c3ccccc3)ccc12]

To a solution of 6-chloro-3-(2-methoxyphenyl)-1-[[2-(trimethylsilyl)ethoxy]methyl]pyrrolo[2,3-b]pyridine (1.9 g, 4.89 mmol) in 1,4-dioxane (20.0 mL) was added diphenylmethanimine (2.7 g, 14.65 mmol), Cs₂CO₃ (4.8 g, 14.65 mmol), BrettPhos (0.5 g, 0.98 mmol) and BrettPhos Pd G3 (0.4 g, 0.49 mmol). The resulting mixture was stirred at 100° C. for 2 h under N₂. After the reaction was completed, the mixture was concentrated under vacuum. The residue was purified by flash column chromatography with ether/ethyl acetate (20/80, v/v) to afford N-[3-(2-methoxyphenyl)-1-[[2-(trimethylsilyl)ethoxy]methyl]pyrrolo[2,3-b]pyridin-6-yl]-1,1-diphenylmethanimine (900.0 mg, 34%) as a yellow oil. LCMS (ESI, m/z): [M+H]⁺=534.2.

### Step 2: Synthesis of 3-(2-methoxyphenyl)-1-[[2-(trimethylsilyl)ethoxy]methyl]pyrrolo[2,3-b]pyridin-6-amine hydrochloride

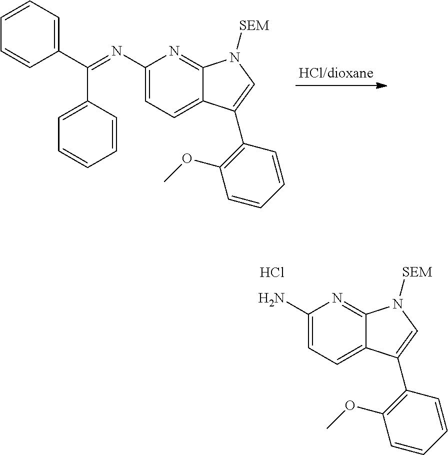 [SMILES: COc1ccccc1-c1cn(COCC[Si](C)(C)C)c2nc(N=C(c3ccccc3)c3ccccc3)ccc12 ; Cl ; COc1ccccc1-c1cn(COCC[Si](C)(C)C)c2nc(N)ccc12]

The solution of N-[3-(2-methoxyphenyl)-1-[[2-(trimethylsilyl)ethoxy]methyl]pyrrolo[2,3-b]pyridin-6-yl]-1,1-diphenylmethanimine (900.0 mg, 1.69 mmol) in HCl/1,4-dioxane (3.0 mL, 4 mol/L) was stirred at room temperature for 2 h. After the reaction was completed, the resulting mixture was concentrated under vacuum. The residue was washed with Et₂O to afford 3-(2-methoxyphenyl)-1-[[2-(trimethylsilyl)ethoxy]methyl]pyrrolo[2,3-b]pyridin-6-amine hydrochloride (400.0 mg, crude) as a yellow solid. LCMS (ESI, m/z): [M+H]⁺=370.2.

### Step 3: Synthesis of 3-[3-(2-methoxyphenyl)-1-[[2-(trimethylsilyl)ethoxy]methyl]pyrrolo[2,3-b]pyridin-6-yl]-1-[2-(4-methylpiperazin-1-yl)ethyl]urea

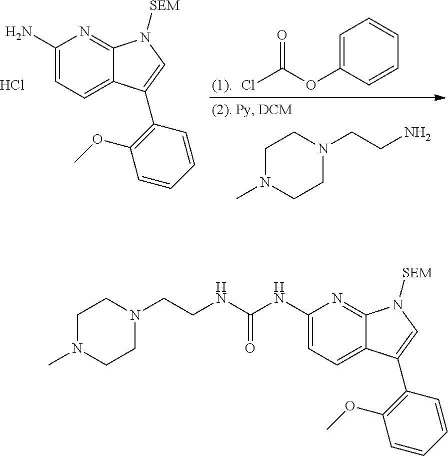

To a solution of 3-(2-methoxyphenyl)-1-[[2-(trimethylsilyl)ethoxy]methyl]pyrrolo[2,3-b]pyridin-6-amine hydrochloride (282.6 mg, 3.57 mmol) in CH₂Cl₂ (3.0 mL) was added pyridine (282.6 mg, 3.57 mmol) and phenyl chloroformate (167.8 mg, 1.07 mmol) at 0° C. The mixture was stirred at room temperature for 16 h. The mixture was concentrated under vacuum. Then pyridine (2.5 mL) and 2-(4-methylpiperazin-1-yl)ethanamine (255.8 mg, 1.79 mmol) were added to the residue. The resulting mixture was stirred at 60° C. for another 2 h. After the reaction was completed, the resulting mixture was concentrated under vacuum. The residue was purified by flash column chromatography with petroleum ether/ethyl acetate (20/80, v/v) to afford 3-[3-(2-methoxyphenyl)-1-[[2-(trimethylsilyl)ethoxy]methyl]pyrrolo[2,3-b]pyridin-6-yl]-1-[2-(4-methylpiperazin-1-yl)ethyl]urea (200.0 mg, 42%) as a yellow solid. LCMS (ESI, m/z): [M+H]⁺=539.3.

### Step 4: Synthesis of 3-[3-(2-methoxyphenyl)-1H-pyrrolo[2,3-b]pyridin-6-yl]-1-[2-(4-methylpiperazin-1-yl)ethyl]urea (Compound 42)

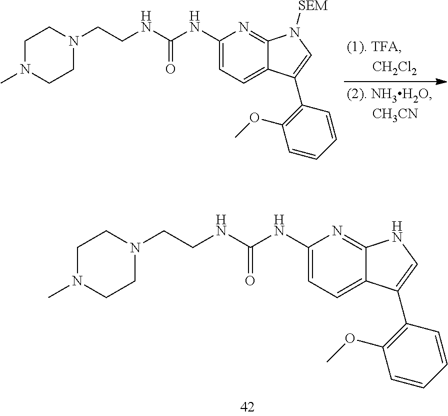 [SMILES: COc1ccccc1-c1cn(COCC[Si](C)(C)C)c2nc(NC(=O)NCCN3CCN(C)CC3)ccc12 ; COc1ccccc1-c1c[nH]c2nc(NC(=O)NCCN3CCN(C)CC3)ccc12]

To a solution of 1-(3-(2-methoxyphenyl)-1-((2-(trimethylsilyl)ethoxy)methyl)-1H-pyrrolo[2,3-b]pyridin-6-yl)-3-(2-(4-methylpiperazin-1-yl)ethyl)urea (180.0 mg, 0.33 mmol) in CH₂Cl₂ (2.0 mL) was added TFA (2.0 mL). The mixture was stirred at room temperature for 2 h. The mixture was concentrated under vacuum. Then ACN (2.0 mL) and NH₃·H₂O (2.0 mL) were added to the residue. The resulting mixture was stirred at room temperature for another 2 h. After the reaction was completed, the resulting mixture was concentrated under vacuum. The residue was purified by Prep-HPLC with the following conditions (Column: YMC-Actus Triart C18 Column, 20×250 mm, 5 um; Mobile Phase A: Water (10 mmol/L NH₄HCO₃), Mobile Phase B: ACN; Flow rate: 60 mL/min; Gradient: 27% B to 57% B in 10 min; 254 nm) to afford 3-[3-(2-methoxyphenyl)-1H-pyrrolo[2,3-b]pyridin-6-yl]-1-[2-(4-methylpiperazin-1-yl)ethyl]urea (18.8 mg, 14%) as a light yellow solid. LCMS (ESI, m/z): [M+H]⁺=409.2. ¹H NMR (300 MHz, CDCl₃): δ 10.07 (s, 1H), 9.73 (s, 1H), 7.94 (d, J=8.4 Hz, 1H), 7.55-7.52 (m, 1H), 7.45-7.42 (m, 2H), 7.35-7.28 (m, 1H), 7.10-7.04 (m, 2H), 6.48 (d, J=8.4 Hz, 1H), 3.90 (s, 3H), 3.67-3.61 (m, 2H), 2.88-2.83 (m, 6H), 2.62-2.59 (m, 4H), 2.40 (s, 3H).

### Example S43. Compound 43

### Step 1: Synthesis of 1-(3-(2-methoxyphenyl)-1-((2-(trimethylsilyl)ethoxy)methyl)-1H-pyrrolo[2,3-b]pyridin-6-yl)-3-(2-morpholinoethyl)urea

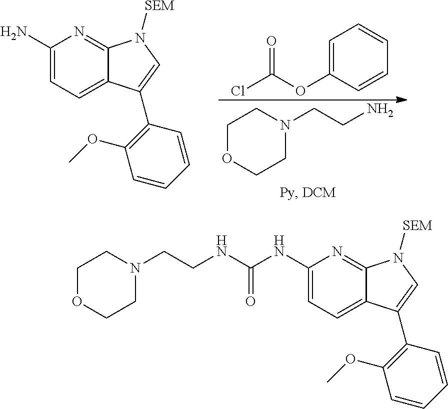

To a solution of 3-(2-methoxyphenyl)-1-[[2-(trimethylsilyl)ethoxy]methyl]pyrrolo[2,3-b]pyridin-6-amine (250.0 mg, 0.68 mmol) in CH₂Cl₂ (5.0 mL) was added Pyridine (107.0 mg, 1.35 mmol) and phenyl chloroformate (127.1 mg, 0.81 mmol) at 0° C. The mixture was stirred at room temperature for 16 h. The mixture was concentrated under vacuum. Then pyridine (2.5 mL) and 2-morpholinoethanamine (264.2 mg, 2.03 mmol) were added to the residue. The resulting mixture was stirred at 60° C. for another 2 h. After the reaction was completed, the resulting mixture was concentrated under vacuum. The residue was purified by flash column chromatography with petroleum ether/ethyl acetate (20/80, v/v) to afford 1-(3-(2-methoxyphenyl)-1-((2-(trimethylsilyl)ethoxy)methyl)-1H-pyrrolo[2,3-b]pyridin-6-yl)-3-(2-morpholinoethyl)urea (200.0 mg, 42%) as a yellow solid. LCMS (ESI, m/z): [M+H]⁺=526.3.

### Step 2: Synthesis of 3-[3-(2-methoxyphenyl)-1H-pyrrolo[2,3-b]pyridin-6-yl]-1-[2-(morpholin-4-yl)ethyl]urea (Compound 43)

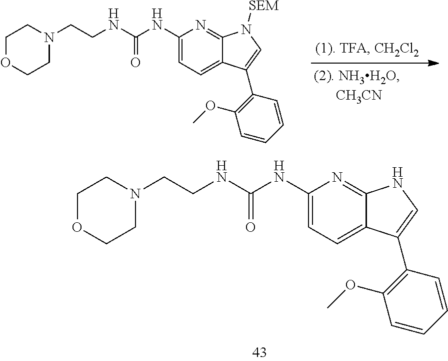 [SMILES: COc1ccccc1-c1cn(COCC[Si](C)(C)C)c2nc(NC(=O)NCCN3CCOCC3)ccc12 ; COc1ccccc1-c1c[nH]c2nc(NC(=O)NCCN3CCOCC3)ccc12]

To a solution of 3-[3-(2-methoxyphenyl)-1-[[2-(trimethylsilyl)ethoxy]methyl]pyrrolo[2,3-b]pyridin-6-yl]-1-[2-(morpholin-4-yl)ethyl]urea (120.0 mg, 0.23 mmol) in CH₂Cl₂ (2.0 mL) was added TFA (1.0 mL). The mixture was stirred at room temperature for 2 h. The mixture was concentrated under vacuum. Then ACN (2.0 mL) and NH₃·H₂O (2.0 mL) were added to the mixture. The resulting mixture was stirred at room temperature for another 2 h. After the reaction was completed, the resulting mixture was concentrated under vacuum. The residue was purified by Prep-HPLC with the following conditions (Column: Xselect CSH OBD Column, 20×250 mm, 5 um; Mobile Phase A: Water (10 mmol/L NH₄HCO₃), Mobile Phase B: ACN; Flow rate: 60 mL/min; Gradient: 25% B to 42% B in 10 min; 254 nm) to afford 3-[3-(2-methoxyphenyl)-1H-pyrrolo[2,3-b]pyridin-6-yl]-1-[2-(morpholin-4-yl)ethyl]urea (31.5 mg, 35%) as a white solid. LCMS (ESI, m/z): [M+H]⁺=396.2. ¹H NMR (300 MHz, DMSO-d₆): δ 11.40 (s, 1H), 9.19 (s, 1H), 8.37 (s, 1H), 7.91 (d, J=8.7 Hz, 1H), 7.53-7.46 (m, 2H), 7.29-7.23 (m, 1H), 7.12-6.99 (m, 3H), 3.81 (s, 3H), 3.61-3.58 (m, 4H), 3.38-3.34 (m, 2H), 2.47-2.42 (m, 6H).

### Example S44. Compound 44

### Step 1: Synthesis of 6-chloro-3-iodo-1H-pyrazolo[3,4-b]pyridine

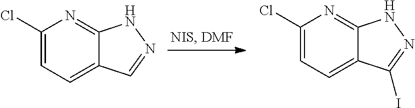 [SMILES: Clc1ccc2cn[nH]c2n1 ; Clc1ccc2c(I)n[nH]c2n1]

A mixture of 6-chloro-1H-pyrazolo[3,4-b]pyridine (1.0 g, 6.51 mmol) and NIS (2.3 g, 0.01 mmol) in DMF (20.0 mL) was stirred at 110° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum to afford 6-chloro-3-iodo-1H-pyrazolo[3,4-b]pyridine (1.7 g, crude) as a yellow solid. LCMS (ESI, m/z): [M+H]⁺=279.9.

### Step 2: Synthesis of 6-chloro-3-iodo-1-[[2-(trimethylsilyl)ethoxy]methyl]pyrazolo[3,4-b]pyridine

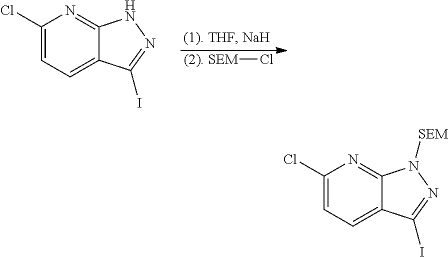

To a solution of 6-chloro-3-iodo-1H-pyrazolo[3,4-b]pyridine (2.7 g, 9.63 mmol) in THF (20.0 mL) was added NaH (0.7 g, 60%) at 0° C. under N₂. The mixture was stirred at 0° C. for 1 h under N₂. Then SEM-Cl (2.4 g, 14.44 mmol) was added to the mixture. The mixture was stirred at room temperature for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (14/86, v/v) to afford 6-chloro-3-iodo-1-[[2-(trimethylsilyl)ethoxy]methyl]pyrazolo[3,4-b]pyridine (750.0 mg, 46%) as a yellow solid. LCMS (ESI, m/z): [M+H]⁺=410.0.

### Step 3: Synthesis of 6-chloro-3-(2-methoxyphenyl)-1-[[2-(trimethylsilyl)ethoxy]methyl]pyrazolo[3,4-b]pyridine

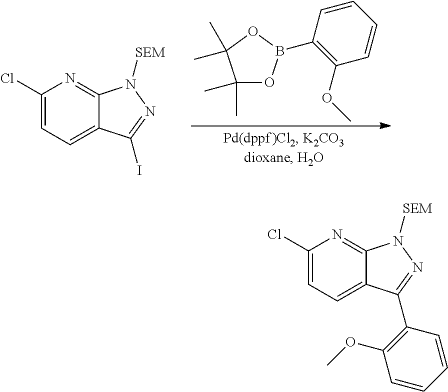 [SMILES: C[Si](C)(C)CCOCn1nc(I)c2ccc(Cl)nc21 ; COc1ccccc1B1OC(C)(C)C(C)(C)O1 ; COc1ccccc1-c1nn(COCC[Si](C)(C)C)c2nc(Cl)ccc12]

A mixture of 6-chloro-3-iodo-1-[[2-(trimethylsilyl)ethoxy]methyl]pyrazolo[3,4-b]pyridine (700.0 mg, 1.71 mmol), 2-methoxyphenylboronic acid (259.6 mg, 1.71 mmol), K₂CO₃ (708.4 mg, 5.13 mmol) and Pd(dppf)Cl₂ (125.0 mg, 0.17 mmol) in dioxane (10.0 mL) and H₂O (2.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (87/13, v/v) to afford 6-chloro-3-(2-methoxyphenyl)-1-[[2-(trimethylsilyl)ethoxy]methyl]pyrazolo[3,4-b]pyridine (444.0 mg, 67%) as a yellow solid. LCMS (ESI, m/z): [M+H]⁺=390.1.

### Step 4: Synthesis of N-[3-(2-methoxyphenyl)-1-[[2-(trimethylsilyl)ethoxy]methyl]pyrazolo[3,4-b]pyridin-6-yl]cyclopropanecarboxamide

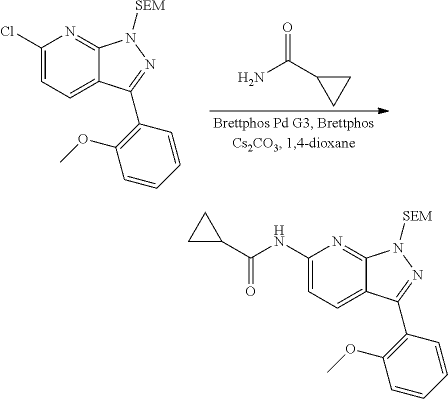 [SMILES: COc1ccccc1-c1nn(COCC[Si](C)(C)C)c2nc(Cl)ccc12 ; NC(=O)C1CC1 ; COc1ccccc1-c1nn(COCC[Si](C)(C)C)c2nc(NC(=O)C3CC3)ccc12]

A mixture of 6-chloro-3-(2-methoxyphenyl)-1-[[2-(trimethylsilyl)ethoxy]methyl]pyrazolo[3,4-b]pyridine (440.0 mg, 1.13 mmol), cyclopropanecarboxamide (288.1 mg, 3.39 mmol), Cs₂CO₃ (1102.9 mg, 3.39 mmol), BrettPhos (121.1 mg, 0.23 mmol) and BrettPhos Pd G3 (102.3 mg, 0.11 mmol) in dioxane (20.0 mL) was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (84/16, v/v) to afford N-[3-(2-methoxyphenyl)-1-[[2-(trimethylsilyl)ethoxy]methyl]pyrazolo[3,4-b]pyridin-6-yl]cyclopropanecarboxamide (483.0 mg, 97%) as a yellow solid. LCMS (ESI, m/z): [M+H]⁺=439.2.

### Step 5: Synthesis of N-[3-(2-methoxyphenyl)-1H-pyrazolo[3,4-b]pyridin-6-yl]cyclopropanecarboxamide (Compound 44)

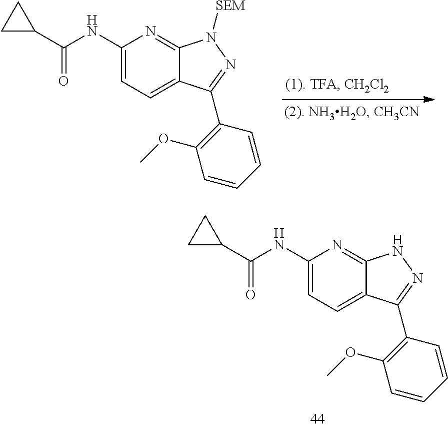

A mixture of N-[3-(2-methoxyphenyl)-1-[[2-(trimethylsilyl)ethoxy]methyl]pyrazolo[3,4-b]pyridin-6-yl]cyclopropanecarboxamide (550.0 mg, 1.25 mmol) and TFA (5.0 mL, 43.85 mmol) in CH₂Cl₂ (2.0 mL) was stirred at room temperature for 2 h. The mixture was evaporated in vacuo. The residue was dissolved in NH₃·H₂O (2.0 mL) and CH₃CN (5.0 mL). The mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 10 min; 254 nm) to afford N-[3-(2-methoxyphenyl)-1H-pyrazolo[3,4-b]pyridin-6-yl]cyclopropanecarboxamide (56.8 mg, 15%) as a white solid. LCMS (ESI, m/z): [M+H]⁺=309.0. ¹H NMR (300 MHz, DMSO-d₆): δ 13.42 (s, 1H), 10.89 (s, 1H), 8.08 (d, J=9.0 Hz, 1H), 7.98 (d, J=9.0 Hz, 1H), 7.63-7.60 (m, 1H), 7.46-7.40 (m, 1H), 7.18 (d, J=7.8 Hz, 1H), 7.09-7.04 (m, 1H), 3.83 (s, 3H), 2.08-2.04 (m, 1H), 0.85-0.82 (m, 4H).

### Example S45. Compound 45

### Step 1: Synthesis of 6-chloro-3-(2,6-dimethoxyphenyl)-1-[[2-(trimethylsilyl)ethoxy]methyl]pyrrolo[2,3-b]pyridine

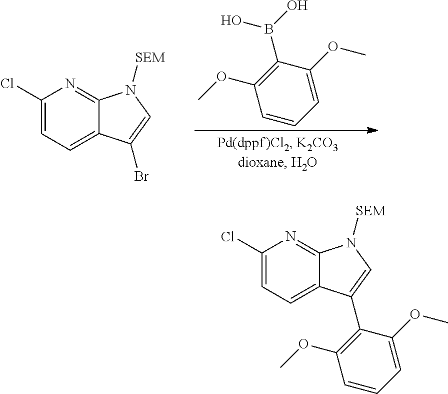

To a solution of 3-bromo-6-chloro-1-[[2-(trimethylsilyl)ethoxy]methyl]pyrrolo[2,3-b]pyridine (600.0 mg, 1.66 mmol) in 1,4-dioxane/H₂O (10.0/2.0 mL) was added 2,6-dimethoxyphenylboronic acid (362.2 mg, 1.99 mmol), K₂CO₃ (458.5 mg, 3.32 mmol) and Pd(dppf)Cl₂ (121.4 mg, 0.17 mmol) at room temperature under N₂. The resulting mixture was stirred at 100° C. for 16 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 6-chloro-3-(2,6-dimethoxyphenyl)-1-[[2-(trimethylsilyl)ethoxy]methyl]pyrrolo[2,3-b]pyridine (93.0 mg, 13%) as a brown oil. LCMS (ESI, m/z): [M+H]⁺=419.1.

### Step 2: Synthesis of N-[3-(2,6-dimethoxyphenyl)-1-[[2-(trimethylsilyl)ethoxy]methyl]pyrrolo[2,3-b]pyridin-6-yl]cyclopropanecarboxamide

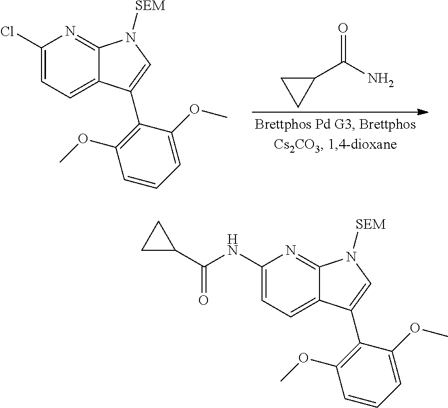

To a solution of 6-chloro-3-(2,6-dimethoxyphenyl)-1-[[2-(trimethylsilyl)ethoxy]methyl]pyrrolo[2,3-b]pyridine (93.0 mg, 0.22 mmol) in 1,4-dioxane (4.0 mL) was added cyclopropanecarboxamide (94.5 mg, 1.11 mmol), Cs₂CO₃ (217.0 mg, 0.67 mmol), BrettPhos (23.8 mg, 0.04 mmol) and BrettPhos Pd G3 (20.1 mg, 0.02 mmol) at room temperature under N₂. The resulting mixture was stirred at 100° C. for 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford N-[3-(2,6-dimethoxyphenyl)-1-[[2-(trimethylsilyl)ethoxy]methyl]pyrrolo[2,3-b]pyridin-6-yl]cyclopropanecarboxamide (100.0 mg, 96%) as a yellow oil. LCMS (ESI, m/z): [M+H]⁺=468.2.

### Step 3: Synthesis of N-[3-(2,6-dimethoxyphenyl)-1H-pyrrolo[2,3-b]pyridin-6-yl]cyclopropanecarboxamide (Compound 45)

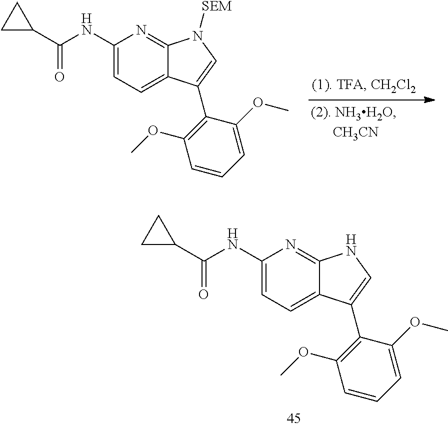 [SMILES: COc1cccc(OC)c1-c1cn(COCC[Si](C)(C)C)c2nc(NC(=O)C3CC3)ccc12 ; COc1cccc(OC)c1-c1c[nH]c2nc(NC(=O)C3CC3)ccc12]

To a solution of N-[3-(2,6-dimethoxyphenyl)-1-[[2-(trimethylsilyl)ethoxy]methyl]pyrrolo[2,3-b]pyridin-6-yl]cyclopropanecarboxamide (260.0 mg, 0.56 mmol) in CH₂Cl₂ (3.0 mL) was added TFA (3.0 mL) at room temperature. The resulting mixture was stirred at room temperature for 4 h. After the reaction was completed, the resulting mixture was concentrated under reduced pressure. The residue was dissolved in CH₃CN (3.0 mL) and NH₃·H₂O (3.0 mL). The resulting mixture was stirred at room temperature for another 16 h. After the reaction was completed, the resulting mixture was concentrated under reduced pressure. The residue was purified by Prep-HPLC with the following conditions (Column: XBridge Shield RP18 OBD Column, 19×250 mm, 10 um; Mobile Phase A: Water (10 mmol/L NH₄HCO₃+0.1% NH₃·H₂O), Mobile Phase B: MeOH—Preparative; Flow rate: 25 mL/min; Gradient: 50% B to 65% B in 9 min; 254 nm) to afford N-[3-(2,6-dimethoxyphenyl)-1H-pyrrolo[2,3-b]pyridin-6-yl]cyclopropanecarboxamide (11.9 mg, 6%) as a white solid. LCMS (ESI, m/z): [M+H]⁺=338.1. ¹H NMR (300 MHz, DMSO-d₆): δ 11.40 (s, 1H), 10.52 (s, 1H), 7.81 (d, J=8.7 Hz, 1H), 7.53 (d, J=8.4 Hz, 1H), 7.27-7.25 (m, 2H), 6.75 (d, J=8.4 Hz, 2H), 3.69 (s, 6H), 2.06-1.99 (m, 1H), 0.82-0.80 (m, 4H).

### Example S46. Compound 46

### Step 1: Synthesis of 6-chloro-3-(2,6-difluorophenyl)-1-((2-(trimethylsilyl)ethoxy)methyl)-1H-pyrrolo[2,3-b]pyridine

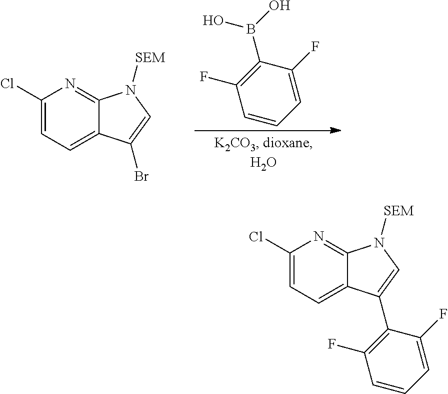 [SMILES: C[Si](C)(C)CCOCn1cc(Br)c2ccc(Cl)nc21 ; OB(O)c1c(F)cccc1F ; C[Si](C)(C)CCOCn1cc(-c2c(F)cccc2F)c2ccc(Cl)nc21]

To a solution of 3-bromo-6-chloro-1-((2-(trimethylsilyl)ethoxy)methyl)-1H-pyrrolo[2,3-b]pyridine (1.0 g, 2.76 mmol) in 1,4-dioxane/H₂O (20.0/4.0 mL) was added (2,6-difluorophenyl)boronic acid (436.5 mg, 2.76 mmol), K₂CO₃ (1.1 g, 8.29 mmol) and Pd(dppf)Cl₂ (202.3 mg, 0.28 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 6-chloro-3-(2,6-difluorophenyl)-1-((2-(trimethylsilyl)ethoxy)methyl)-1H-pyrrolo[2,3-b]pyridine (160.0 mg, 12%) as a yellow oil. LCMS (ESI, m/z): [M+H]⁺=395.1.

### Step 2: Synthesis of N-(3-(2,6-difluorophenyl)-1-((2-(trimethylsilyl)ethoxy)methyl)-1H-pyrrolo[2,3-b]pyridin-6-yl)cyclopropanecarboxamide

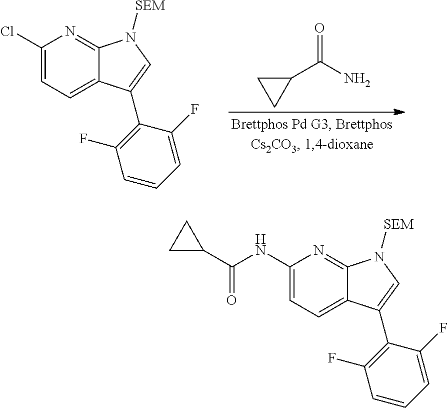

To a solution of 6-chloro-3-(2,6-difluorophenyl)-1-((2-(trimethylsilyl)ethoxy)methyl)-1H-pyrrolo[2,3-b]pyridine (250.0 mg, 0.63 mmol) in 1,4-dioxane (10.0 mL) was added cyclopropanecarboxamide (269.4 mg, 3.17 mmol), Cs₂CO₃ (618.8 mg, 1.90 mmol), BrettPhos (68.0 mg, 0.13 mmol) and BrettPhos Pd G3 (57.4 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (83/17, v/v) to afford N-(3-(2,6-difluorophenyl)-1-((2-(trimethylsilyl)ethoxy)methyl)-1H-pyrrolo[2,3-b]pyridin-6-yl)cyclopropanecarboxamide (50.0 mg, 18%) as a yellow oil. LCMS (ESI, m/z): [M+H]⁺=444.2.

### Step 3: Synthesis of N-(3-(2,6-difluorophenyl)-1H-pyrrolo[2,3-b]pyridin-6-yl)cyclopropanecarboxamide (Compound 46)

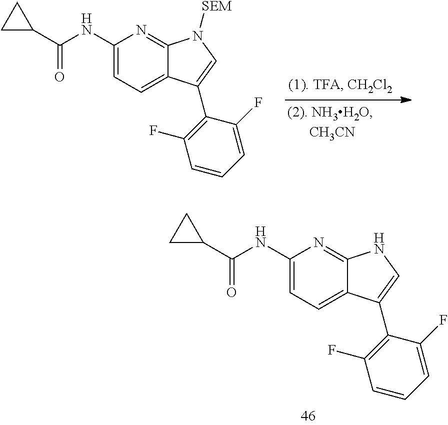 [SMILES: C[Si](C)(C)CCOCn1cc(-c2c(F)cccc2F)c2ccc(NC(=O)C3CC3)nc21 ; O=C(Nc1ccc2c(-c3c(F)cccc3F)c[nH]c2n1)C1CC1]

To a solution of N-(3-(2,6-difluorophenyl)-1-((2-(trimethylsilyl)ethoxy)methyl)-1H-pyrrolo[2,3-b]pyridin-6-yl)cyclopropanecarboxamide (50.0 mg, 0.11 mmol) in CH₂Cl₂ (2.0 mL) was added TFA (2.0 mL) at room temperature. The resulting mixture was stirred at room temperature for 4 h. The resulting mixture was concentrated under reduced pressure. The residue was dissolved in CH₃CN (2.0 mL) and NH₃·H₂O (2.0 mL). The resulting mixture was stirred at room temperature for another 2 h. After the reaction was completed, the resulting mixture was concentrated under reduced pressure. The residue was purified by Prep-HPLC with the following conditions (Column: XBridge BEH C18 OBD Prep Column, 5 μm, 19 mm 250 mm; Mobile Phase A: Water (10 mmol/L NH₄HCO₃), Mobile Phase B: ACN; Flow rate: 25 mL/min; Gradient: 47% B to 51% B in 12 min; 254 nm) to afford N-(3-(2,6-difluorophenyl)-1H-pyrrolo[2,3-b]pyridin-6-yl)cyclopropanecarboxamide (11.2 mg, 32%) as a white solid. LCMS (ESI, m/z): [M+H]⁺=314.1. ¹H NMR (400 MHz, DMSO-d₆): δ 11.86 (s, 1H), 10.61 (s, 1H), 7.90 (d, J=8.4 Hz, 1H), 7.76 (d, J=8.4 Hz, 1H), 7.59 (d, J=1.2 Hz, 1H), 7.46-7.34 (m, 1H), 7.22-7.18 (m, 2H), 2.10-1.98 (m, 1H), 0.82-0.76 (m, 4H).

### Example S47. Compound 47

### Step 1: Synthesis of 3-(6-chloro-1-[[2-(trimethylsilyl)ethoxy]methyl]pyrrolo[2,3-b]pyridin-3-yl)-2-ethoxypyridine

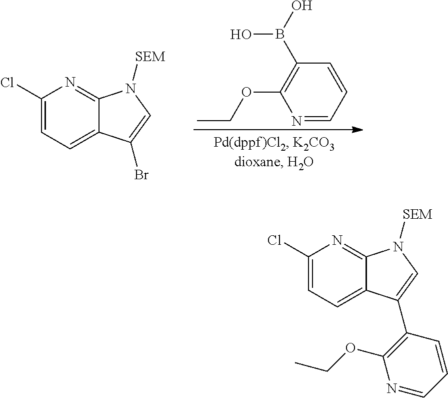 [SMILES: C[Si](C)(C)CCOCn1cc(Br)c2ccc(Cl)nc21 ; CCOc1ncccc1B(O)O ; CCOc1ncccc1-c1cn(COCC[Si](C)(C)C)c2nc(Cl)ccc12]

To a mixture of 3-bromo-6-chloro-1-[[2-(trimethylsilyl)ethoxy]methyl]pyrrolo[2,3-b]pyridine (300.0 mg, 0.82 mmol) and 2-ethoxypyridin-3-ylboronic acid (207.7 mg, 1.24 mmol) in dioxane/H₂O (5.0/0.5 mL) was added Pd(dppf)Cl₂ (60.6 mg, 0.08 mmol) and K₂CO₃ (343.8 mg, 2.48 mmol) at room temperature. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 3-(6-chloro-1-[[2-(trimethylsilyl)ethoxy]methyl]pyrrolo[2,3-b]pyridin-3-yl)-2-ethoxypyridine (230.0 mg, 69%) as a yellow solid. LCMS (ESI, m/z): [M+H]⁺=404.1.

### Step 2: Synthesis of N-(3-(2-ethoxypyridin-3-yl)-1-((2-(trimethylsilyl)ethoxy)methyl)-1H-pyrrolo[2,3-b]pyridin-6-yl)cyclopropanecarboxamide

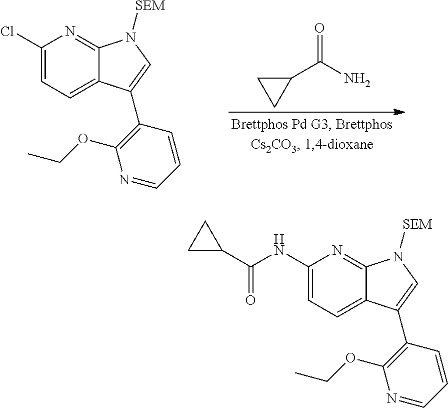 [SMILES: CCOc1ncccc1-c1cn(COCC[Si](C)(C)C)c2nc(Cl)ccc12 ; NC(=O)C1CC1 ; CCOc1ncccc1-c1cn(COCC[Si](C)(C)C)c2nc(NC(=O)C3CC3)ccc12]

To a mixture of 6-chloro-3-(2-ethoxypyridin-3-yl)-1-((2-(trimethylsilyl)ethoxy)methyl)-1H-pyrrolo[2,3-b]pyridine (260.0 mg, 0.62 mmol) and cyclopropanecarboxamide (211.2 mg, 2.48 mmol) in dioxane (4.0 mL) was added Brettphos Pd G3 (56.2 mg, 0.06 mmol), BrettPhos (66.6 mg, 0.12 mmol) and Cs₂CO₃ (606.5 mg, 1.82 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-(3-(2-ethoxypyridin-3-yl)-1-((2-(trimethylsilyl)ethoxy)methyl)-1H-pyrrolo[2,3-b]pyridin-6-yl)cyclopropanecarboxamide (260.0 mg, 89%) as a white solid. LCMS (ESI, m/z): [M+H]⁺=453.2

### Step 3: Synthesis of N-[3-(2-ethoxypyridin-3-yl)-1H-pyrrolo[2,3-b]pyridin-6-yl]cyclopropanecarboxamide (Compound 47)

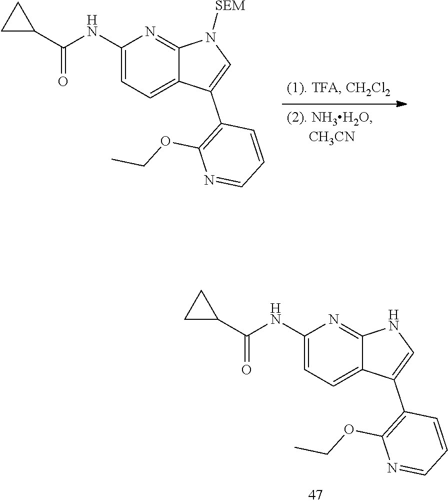 [SMILES: CCOc1ncccc1-c1cn(COCC[Si](C)(C)C)c2nc(NC(=O)C3CC3)ccc12 ; CCOc1ncccc1-c1c[nH]c2nc(NC(=O)C3CC3)ccc12]

To a solution of N-[3-(2-ethoxypyridin-3-yl)-1-[[2-(trimethylsilyl)ethoxy]methyl]pyrrolo[2,3-b] pyridin-6-yl]cyclopropanecarboxamide (270.0 mg, 0.59 mmol) in DCM (4.0 mL) was added TFA (2.0 mL) at room temperature. The resulting mixture was stirred at room temperature for 2 h. The resulting mixture was concentrated under reduced pressure. To the above mixture was added NH₃·H₂O (4.0 mL) and ACN (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol NH₄HCO₃), Mobile Phase B: ACN; Flow rate: 60 mL/min; Gradient: 32% B to 62% B in 10 min; 254 nm) to afford N-[3-(2-ethoxypyridin-3-yl)-1H-pyrrolo[2,3-b]pyridin-6-yl]cyclopropanecarboxamide (46.7 mg, 24%) as a white solid. LCMS (ESI, m/z): [M+H]⁺=323.2. ¹H NMR (300 MHz, DMSO-d₆): δ 11.68 (s, 1H), 10.62 (s, 1H), 8.13 (d, J=9.0 Hz, 1H), 8.07-8.04 (m, 1H), 8.01-7.92 (m, 2H), 7.74 (d, J=2.7 Hz, 1H), 7.08-7.04 (m, 1H), 4.45-4.38 (m, 2H), 2.07-2.03 (m, 1H), 1.38-1.33 (m, 3H), 0.84-0.79 (m, 4H).

### Example S48. Compound 48

### Step 1: Synthesis of 3-bromo-2-(difluoromethoxy)pyridine

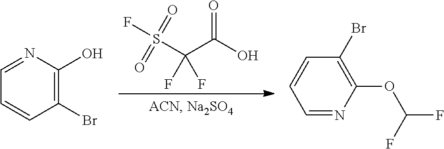 [SMILES: Oc1ncccc1Br ; O=C(O)C(F)(F)S(=O)(=O)F ; FC(F)Oc1ncccc1Br]

To a solution of 3-bromopyridin-2-ol (5.0 g, 28.73 mmol) in ACN (10.0 mL) was added 2,2-difluoro-2-(fluorosulfonyl)acetic acid (6.1 g, 34.48 mmol) and Na₂SO₄ (4.9 g, 34.48 mmol) at room temperature under N₂. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (94/6, v/v) to afford 3-bromo-2-(difluoromethoxy)pyridine (3.2 g, 50%) as a colorless oil. LCMS (ESI, m/z): [M+H]⁺=223.9.

### Step 2: Synthesis of 3-(6-chloro-1-[[2-(trimethylsilyl)ethoxy]methyl]pyrrolo[2,3-b]pyridin-3-yl)-2-(difluoromethoxy)pyridine

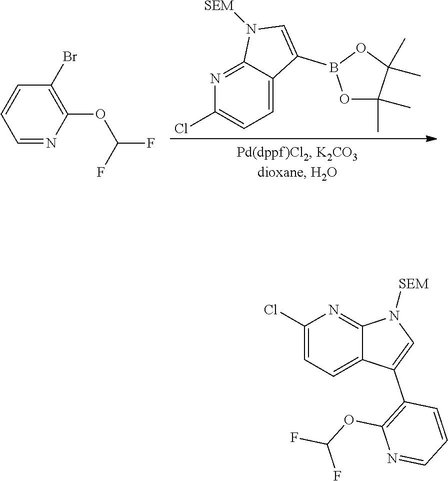

To a solution of 3-bromo-2-(difluoromethoxy)pyridine (200.0 mg, 0.89 mmol) in 1,4-dioxane/H₂O (5.0/1.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (574.5 mg, 1.34 mmol), K₂CO₃ (370.2 mg, 2.68 mmol) and Pd(dppf)Cl₂ (145.8 mg, 0.18 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (83/17, v/v) to afford 3-(6-chloro-1-[[2-(trimethylsilyl)ethoxy]methyl]pyrrolo[2,3-b]pyridin-3-yl)-2-(difluoromethoxy)pyridine (200.0 mg, 52%) as a yellow oil. LCMS (ESI, m/z): [M+H]⁺=426.1.

### Step 3: Synthesis of N-[3-[2-(difluoromethoxy)pyridin-3-yl]-1-[[2-(trimethylsilyl)ethoxy]methyl]pyrrolo[2,3-b]pyridin-6-yl]cyclopropanecarboxamide

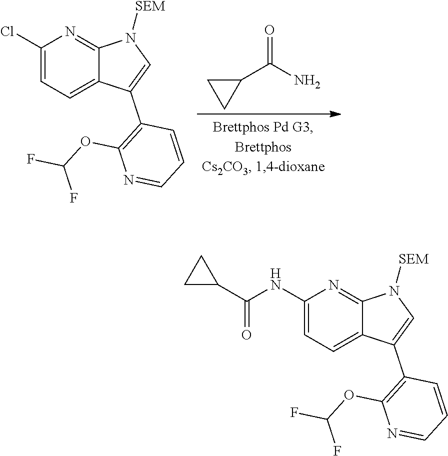 [SMILES: C[Si](C)(C)CCOCn1cc(-c2cccnc2OC(F)F)c2ccc(Cl)nc21 ; NC(=O)C1CC1 ; C[Si](C)(C)CCOCn1cc(-c2cccnc2OC(F)F)c2ccc(NC(=O)C3CC3)nc21]

To a solution of 3-(6-chloro-1-[[2-(trimethylsilyl)ethoxy]methyl]pyrrolo[2,3-b]pyridin-3-yl)-2-(difluoromethoxy)pyridine (200.0 mg, 0.47 mmol) in dioxane (3.0 mL) was added cyclopropanecarboxamide (119.9 mg, 1.41 mmol), BrettPhos (50.4 mg, 0.09 mmol), Cs₂CO₃ (459.0 mg, 1.41 mmol) and BrettPhos Pd G3 (42.6 mg, 0.05 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford N-[3-[2-(difluoromethoxy)pyridin-3-yl]-1-[[2-(trimethylsilyl)ethoxy]methyl]pyrrolo[2,3-b]pyridin-6-yl]cyclopropanecarboxamide (200.0 mg, 89%) as a white solid. LCMS (ESI, m/z): [M+H]⁺=475.2.

### Step 4: Synthesis of N-[3-[2-(difluoromethoxy)pyridin-3-yl]-1H-pyrrolo[2,3-b]pyridin-6-yl]cyclopropanecarboxamide (Compound 48)

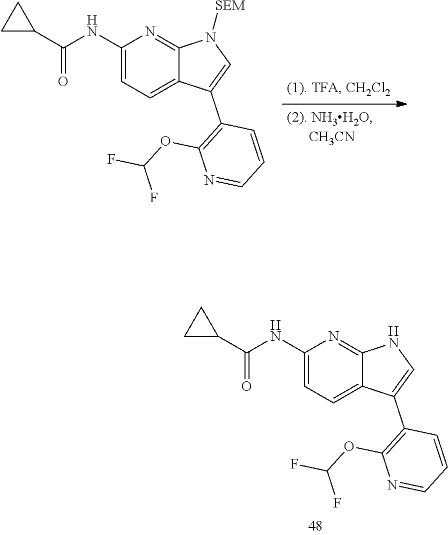 [SMILES: C[Si](C)(C)CCOCn1cc(-c2cccnc2OC(F)F)c2ccc(NC(=O)C3CC3)nc21 ; O=C(Nc1ccc2c(-c3cccnc3OC(F)F)c[nH]c2n1)C1CC1]

To a solution of N-[3-[2-(difluoromethoxy)pyridin-3-yl]-1-[[2-(trimethylsilyl)ethoxy]methyl]pyrrolo[2,3-b]pyridin-6-yl]cyclopropanecarboxamide (190.0 mg, 0.40 mmol) in CH₂Cl₂ (2.0 mL) was added TFA (2.0 mL) at room temperature. The resulting mixture was stirred at room temperature for 3 h. The resulting mixture was concentrated under reduced pressure. The residue was dissolved in CH₃CN (2.0 mL) and NH₃·H₂O (2.0 mL). The resulting mixture was stirred at room temperature for another 16 h. After the reaction was completed, the resulting mixture was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 34% B to 64% B in 10 min; 254 nm) to afford N-[3-[2-(difluoromethoxy)pyridin-3-yl]-1H-pyrrolo[2,3-b]pyridin-6-yl]cyclopropanecarboxamide (45.2 mg, 28%) as a white solid. LCMS (ESI, m/z): [M+H]⁺=345.2. ¹H NMR (400 MHz, DMSO-d₆): δ 11.83 (s, 1H), 10.64 (s, 1H), 8.16 (d, J=6.0 Hz, 2H), 8.10 (d, J=8.8 Hz, 1H), 7.99-7.63 (m, 3H), 7.37-7.34 (m, 1H), 2.09-1.99 (m, 1H), 0.83-0.79 (m, 4H).

### Example S49. Compound 49

### Step 1: Synthesis of (1S,2S)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide

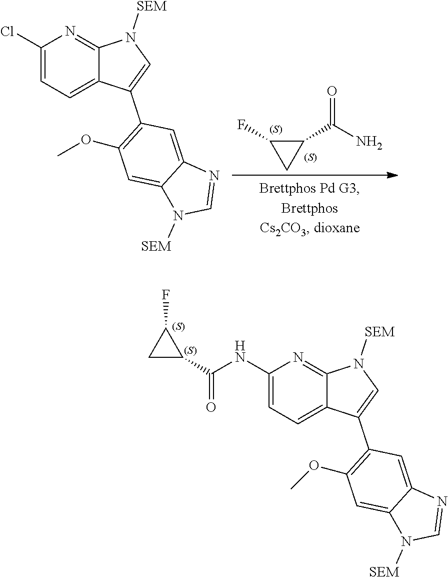

To a solution of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,3-benzodiazole (200.0 mg, 0.18 mmol) in 1,4-dioxane (8.0 mL) was added (1S,2S)-2-fluorocyclopropane-1-carboxamide (92.2 mg, 0.89 mmol), BrettPhos (19.2 mg, 0.04 mmol), Cs₂CO₃ (174.8 mg, 0.54 mmol) and BrettPhos Pd G3 (16.2 mg, 0.02 mmol) at room temperature under N₂. The resulting mixture was stirred with microwave at 100° C. for 2 h under N₂. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford (1S,2S)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (50.0 mg, 45%) as a yellow oil. LCMS (ESI, m/z): [M+H]⁺=626.3.

### Step 2: Synthesis of (1S,2S)-2-fluoro-N-[3-(6-methoxy-1H-1,3-benzodiazol-5-yl)-1H-pyrrolo[2,3-b]pyridin-6-yl]cyclopropane-1-carboxamide: (Compound 49)

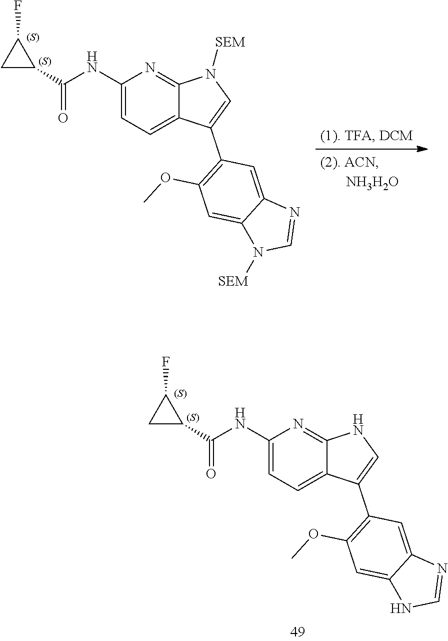

To a solution of (1S,2S)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (215.0 mg, 0.34 mmol) in DCM (6.0 mL) was added TFA (6.0 mL) at room temperature. The resulting mixture was stirred at room temperature for 2 h. The resulting mixture was concentrated under reduced pressure. The residue was dissolved in CH₃CN (2.0 mL) and NH₃·H₂O (2.0 mL). The resulting mixture was stirred at room temperature for another 16 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 53% B to 43% B in 7 min; 254 nm) to afford (1S,2S)-2-fluoro-N-[3-(6-methoxy-1H-1,3-benzodiazol-5-yl)-1H-pyrrolo[2,3-b]pyridin-6-yl]cyclopropane-1-carboxamide (6.9 mg, 6%) as a white solid. LCMS (ESI, m/z): [M+H]⁺=366.1. ¹H NMR (300 MHz, DMSO-d₆): δ 12.35-12.26 (br, 1H), 11.50 (s, 1H), 10.61 (s, 1H), 8.16 (s, 1H), 7.96-7.87 (m, 2H), 7.64 (s, 1H), 7.52 (d, J=2.1 Hz, 1H), 7.24 (s, 1H), 5.06-4.78 (m, 1H), 3.82 (s, 3H), 2.27-2.21 (m, 1H), 1.71-1.61 (m, 1H), 1.19-1.15 (m, 1H).

### Example S50. Compound 50

### Step 1: Synthesis of (1S,2S)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide

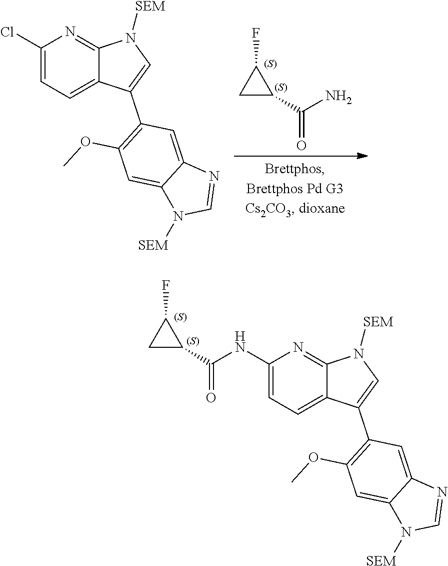

To a solution of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]indazole (300.0 mg, 0.54 mmol) in 1,4-dioxane (12.0 mL) was added (1S,2S)-2-fluorocyclopropane-1-carboxamide (276.5 mg, 2.68 mmol), BrettPhos (57.6 mg, 0.11 mmol), Cs₂CO₃ (524.3 mg, 1.61 mmol) and BrettPhos Pd G3 (48.6 mg, 0.05 mmol) at room temperature under N₂. The resulting mixture was stirred with microwave at 120° C. for 1.5 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide (100.0 mg, 30%) as a yellow oil. LCMS (ESI, m/z): [M+H]⁺=626.3.

### Step 2: Synthesis of (1S,2S)-2-fluoro-N-[3-(6-methoxy-1H-indazol-5-yl)-1H-pyrrolo[2,3-b]pyridin-6-yl]cyclopropane-1-carboxamide (Compound 50)

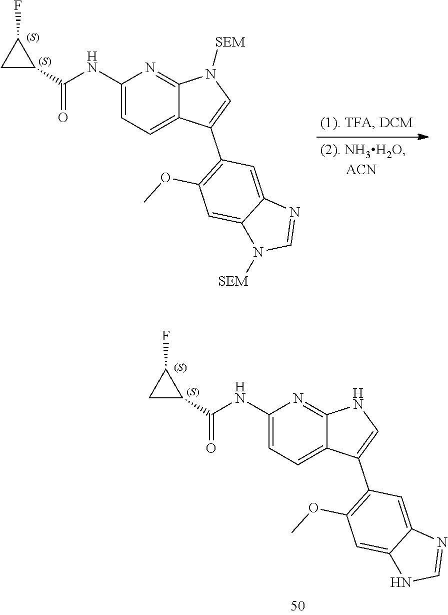

To a solution of (1S,2S)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide (100.0 mg, 0.16 mmol) in DCM (4.0 mL) was added TFA (4.0 mL) at room temperature. The resulting mixture was stirred at room temperature for 2 h. The resulting mixture was concentrated under reduced pressure. The residue was dissolved in CH₃CN (2.0 mL) and NH₃·H₂O (2.0 mL). The resulting mixture was stirred at room temperature for another 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 7 min; 254 nm) to afford (1S,2S)-2-fluoro-N-[3-(6-methoxy-1H-indazol-5-yl)-1H-pyrrolo[2,3-b]pyridin-6-yl]cyclopropane-1-carboxamide (12.3 mg, 21%) as a white solid. LCMS (ESI, m/z): [M+H]⁺=366.2. ¹H NMR (300 MHz, DMSO-d₆): δ 12.82 (s, 1H), 11.50 (s, 1H), 10.61 (s, 1H), 7.98-7.80 (m, 4H), 7.52 (d, J=2.7 Hz, 1H), 7.06 (s, 1H), 5.17-4.73 (m, 1H), 3.87 (s, 3H), 2.32-2.18 (m, 1H), 1.78-1.52 (m, 1H), 1.29-1.01 (m, 1H).

### Example S51. Compound 51

### Step 1: Synthesis of (1R,2R)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide

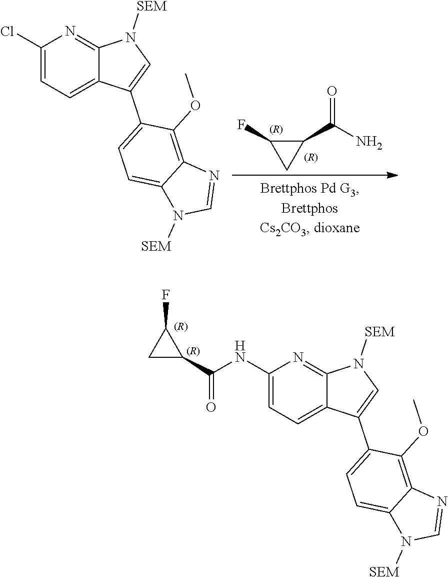

To a solution of 5-(6-chloro-1-[[2-(trimethylsilyl)ethoxy]methyl]pyrrolo[2,3-b]pyridin-3-yl)-4-methoxy-1-[[2-(trimethylsilyl)ethoxy]methyl]-1,3-benzodiazole (200.0 mg, 0.36 mmol) in 1,4-dioxane (6.0 mL) was added (1R,2R)-2-fluorocyclopropane-1-carboxamide (184.4 mg, 1.79 mmol), BrettPhos (38.4 mg, 0.07 mmol), Cs₂CO₃ (349.6 mg, 1.07 mmol) and BrettPhos Pd G3 (32.4 mg, 0.04 mmol) at room temperature under N₂. The resulting mixture was irradiated with microwave radiation at 120° C. for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ether/ethyl acetate (2/1, v/v) to afford (1R,2R)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (92.0 mg, 41%) as a yellow oil. LCMS (ESI, m/z): [M+H]⁺=626.3.

### Step 2: Synthesis of (1R,2R)-2-fluoro-N-[3-(4-methoxy-1H-1,3-benzodiazol-5-yl)-1H-pyrrolo[2,3-b]pyridin-6-yl]cyclopropane-1-carboxamide (Compound 51)

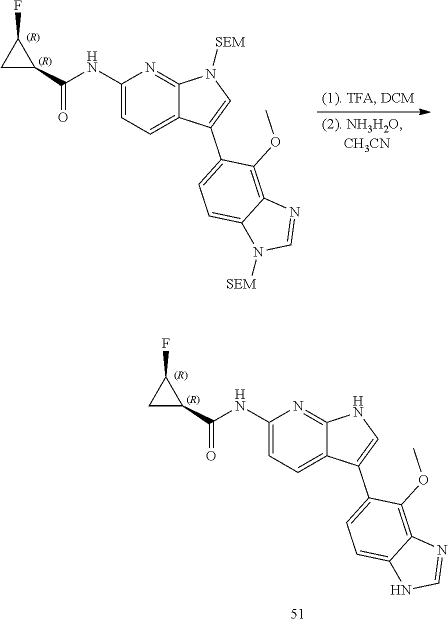

To a solution of (1R,2R)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (40.0 mg, 0.06 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL). The resulting mixture was stirred at room temperature for another 16 h. After the reaction was completed, the resulting mixture was concentrated under reduced pressure. The residue was purified by Prep-HPLC with the following conditions (Column: YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 22% B to 52% B in 7 min; 254 nm) to afford (1R,2R)-2-fluoro-N-[3-(4-methoxy-1H-1,3-benzodiazol-5-yl)-1H-pyrrolo[2,3-b]pyridin-6-yl]cyclopropane-1-carboxamide (5.2 mg, 22%) as a white solid. LCMS (ESI, m/z): [M+H]⁺=366.2. ¹H NMR (300 MHz, DMSO-d₆): δ 12.60 (s, 1H), 11.60 (s, 1H), 10.74 (s, 1H), 8.28 (s, 1H), 8.12-7.98 (m, 2H), 7.58 (s, 1H), 7.45 (d, J=8.4 Hz, 1H), 7.35 (d, J=8.1 Hz, 1H), 5.15-4.91 (m, 1H), 4.32 (s, 2H), 3.75 (s, 1H), 2.38-2.31 (m, 1H), 1.81-1.71 (m, 1H), 1.34-1.20 (m, 1H).

### Example S52. Compound 52

### Step 1: Synthesis of 5-bromo-6-methoxy-1-[[2-(trimethylsilyl)ethoxy]methyl]-1,3-benzodiazole

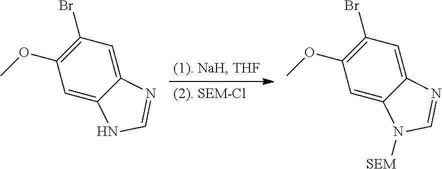 [SMILES: COc1cc2[nH]cnc2cc1Br ; COc1cc2c(cc1Br)ncn2COCC[Si](C)(C)C]

To a solution of 5-bromo-6-methoxy-1H-1,3-benzodiazole (1.0 g, 4.40 mmol) in THE (20.0 mL) was added NaH (0.2 g, 60%) at 0° C. under N₂. The resulting mixture was stirred at 0° C. for 1 h. Then SEM-Cl (0.9 g, 5.28 mmol) was added to the mixture. The resulting mixture was stirred at 0° C. for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (1/1, v/v) to afford 5-bromo-6-methoxy-1-[[2-(trimethylsilyl)ethoxy]methyl]-1,3-benzodiazole (1.5 g, 95%) as a yellow oil. LCMS (ESI, m/z): [M+H]⁺=357.1.

### Step 2: Synthesis of 6-methoxy-5-(4,4,5,5-tetramethyl-1,3,2-dioxaborolan-2-yl)-1-[[2-(trimethylsilyl)ethoxy]methyl]-1,3-benzodiazole

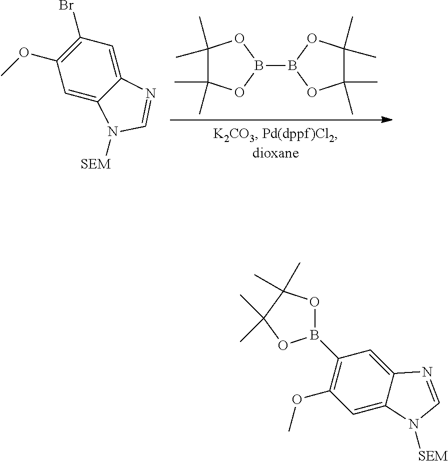

To a solution of 5-bromo-6-methoxy-1-[[2-(trimethylsilyl)ethoxy]methyl]-1,3-benzodiazole (750.0 mg, 2.10 mmol) in 1,4-dioxane (20.0 mL) was added bis(pinacolato)diboron (639.6 mg, 2.52 mmol), KOAc (618.0 mg, 6.30 mmol) and Pd(dppf)Cl₂ (153.6 mg, 0.21 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (6/1, v/v) to afford 6-methoxy-5-(4,4,5,5-tetramethyl-1,3,2-dioxaborolan-2-yl)-1-[[2-(trimethylsilyl)ethoxy]methyl]-1,3-benzodiazole (420.0 mg, 49%) as a yellow solid. LCMS (ESI, m/z): [M+H]⁺=405.2.

### Step 3: Synthesis of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,3-benzodiazole

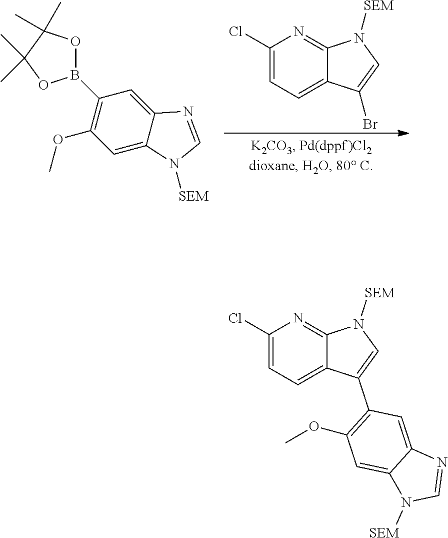 [SMILES: COc1cc2c(cc1B1OC(C)(C)C(C)(C)O1)ncn2COCC[Si](C)(C)C ; C[Si](C)(C)CCOCn1cc(Br)c2ccc(Cl)nc21 ; COc1cc2c(cc1-c1cn(COCC[Si](C)(C)C)c3nc(Cl)ccc13)ncn2COCC[Si](C)(C)C]

To a solution of 6-methoxy-5-(4,4,5,5-tetramethyl-1,3,2-dioxaborolan-2-yl)-1-[[2-(trimethylsilyl)ethoxy]methyl]-1,3-benzodiazole (870.0 mg, 2.15 mmol) in 1,4-dioxane/H₂O (15.0/3.0 mL) was added 3-bromo-6-chloro-1-[[2-(trimethylsilyl)ethoxy]methyl]pyrrolo[2,3-b]pyridine (778.2 mg, 2.15 mmol), K₂CO₃ (892.0 mg, 6.45 mmol) and Pd(dppf)Cl₂ (314.8 mg, 0.43 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,3-benzodiazole (590.0 mg, 49%) as a yellow oil. LCMS (ESI, m/z): [M+H]⁺=559.2.

### Step 4: Synthesis of (1R,2R)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide

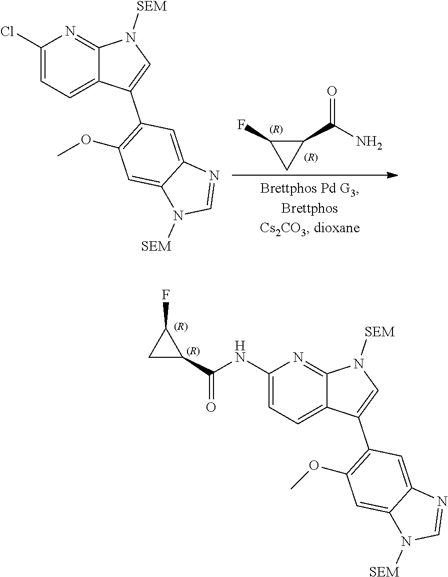

To a solution of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,3-benzodiazole (540.0 mg, 0.97 mmol) in 1,4-dioxane (10.0 mL) was added (1R,2R)-2-fluorocyclopropane-1-carboxamide (497.7 mg, 4.83 mmol), Cs₂CO₃ (943.8 mg, 2.90 mmol), BrettPhos (103.7 mg, 0.19 mmol) and BrettPhos Pd G3 (87.5 mg, 0.10 mmol) at room temperature under N₂. The reaction mixture was irradiated with microwave radiation at 100° C. for 2 h. After the reaction was completed, the mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1R,2R)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (180.0 mg, 29%) as a yellow oil. LCMS (ESI, m/z): [M+H]⁺=626.3.

### Step 5: Synthesis of (1R,2R)-2-fluoro-N-[3-(6-methoxy-1H-1,3-benzodiazol-5-yl)-1H-pyrrolo[2,3-b]pyridin-6-yl]cyclopropane-1-carboxamide (Compound 52)

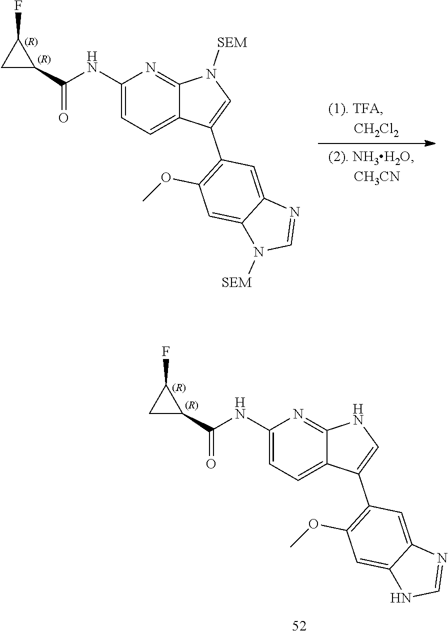

To a solution of (1R,2R)-2-fluoro-N-[3-(6-methoxy-1-[[2-(trimethylsilyl)ethoxy]methyl]-1,3-benzodiazol-5-yl)-1-[[2-(trimethylsilyl)ethoxy]methyl]pyrrolo[2,3-b]pyridin-6-yl]cyclopropane-1-carboxamide (180.0 mg, 0.29 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 22% B to 52% B in 7 min; 254 nm) to afford (1R,2R)-2-fluoro-N-[3-(6-methoxy-1H-1,3-benzodiazol-5-yl)-1H-pyrrolo[2,3-b]pyridin-6-yl]cyclopropane-1-carboxamide (20.1 mg, 19%) as a white solid. LCMS (ESI, m/z): [M+H]⁺=366.1. ¹H NMR (300 MHz, DMSO-d₆): δ 12.25 (s, 1H), 11.50 (s, 1H), 10.62 (s, 1H), 8.11 (s, 1H), 7.97-7.88 (m, 2H), 7.72-7.57 (m, 1H), 7.53 (s, 1H), 7.41-7.10 (m, 1H), 5.03-4.81 (m, 1H), 3.83 (s, 3H), 2.29-2.18 (m, 1H), 1.71-1.60 (m, 1H), 1.19-1.12 (m, 1H).

### Example S53. Compound 53

### Step 1: Synthesis of 5-bromo-6-methoxy-1-[[2-(trimethylsilyl)ethoxy]methyl]indazole

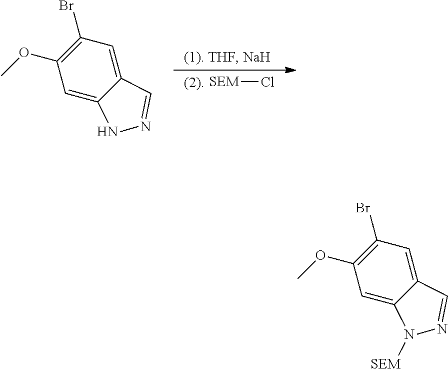 [SMILES: COc1cc2[nH]ncc2cc1Br ; C[Si](C)(C)CCOCCl ; COc1cc2c(cnn2COCC[Si](C)(C)C)cc1Br]

To a solution of 5-bromo-6-methoxy-1H-indazole (3.0 g, 6.60 mmol) in THF (45.0 mL) was added NaH (475.6 mg, 60%) at 0° C. under N₂. The resulting mixture was stirred at 0° C. for 1 h. Then SEM-Cl (4.4 g, 26.43 mmol) was added dropwise to the mixture at 0° C. under N₂. The resulting mixture was stirred at 0° C. for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford 5-bromo-6-methoxy-1-[[2-(trimethylsilyl)ethoxy]methyl]indazole (3.0 g, 64%) as a yellow solid. LCMS (ESI, m/z): [M+H]⁺=357.1.

### Step 2: Synthesis of 6-methoxy-5-(4,4,5,5-tetramethyl-1,3,2-dioxaborolan-2-yl)-1-[[2-(trimethylsilyl)ethoxy]methyl]indazole

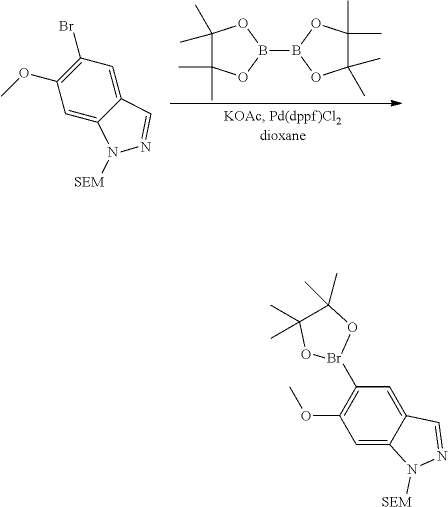

To a solution of 5-bromo-6-methoxy-1-[[2-(trimethylsilyl)ethoxy]methyl]indazole (2.5 g, 6.99 mmol) in dioxane (40.0 mL) was added bis(pinacolato)diboron (2.6 g, 10.54 mmol), KOAc (2.0 g, 20.99 mmol) and Pd(dppf)Cl₂ (511.9 mg, 0.70 mmol) at room temperature under N₂. The resulting mixture was stirred at 100° C. for 16 h. After the reaction was completed, the resulting mixture was concentrated under reduced pressure. The residue was purified by flash column chromatography with petroleum ether/ethyl acetate (1/1, v/v) to afford 6-methoxy-5-(4,4,5,5-tetramethyl-1,3,2-dioxaborolan-2-yl)-1-[[2-(trimethylsilyl)ethoxy]methyl]indazole (1.1 g, 39%) as a yellow oil. LCMS (ESI, m/z): [M+H]⁺=405.2.

### Step 3: Synthesis of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]indazole

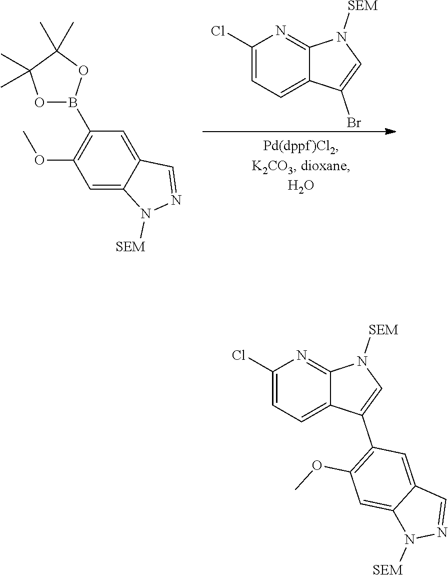 [SMILES: COc1cc2c(cnn2COCC[Si](C)(C)C)cc1B1OC(C)(C)C(C)(C)O1 ; C[Si](C)(C)CCOCn1cc(Br)c2ccc(Cl)nc21 ; COc1cc2c(cnn2COCC[Si](C)(C)C)cc1-c1cn(COCC[Si](C)(C)C)c2nc(Cl)ccc12]

To a solution of 6-methoxy-5-(4,4,5,5-tetramethyl-1,3,2-dioxaborolan-2-yl)-1-[[2-(trimethylsilyl)ethoxy]methyl]indazole (1.0 g, 2.47 mmol) in dioxane/H₂O (10.0/1.0 mL) was added Pd(dppf)Cl₂ (180.9 mg, 0.27 mmol), 3-bromo-6-chloro-1-[[2-(trimethylsilyl)ethoxy]methyl]pyrrolo[2,3-b]pyridine (894.5 mg, 2.47 mmol) and K₂CO₃ (1025.9 mg, 7.49 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]indazole (500.0 mg, 48%) as a yellow oil. LCMS (ESI, m/z): [M+H]⁺=559.2.

### Step 4: Synthesis of (1R,2R)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide

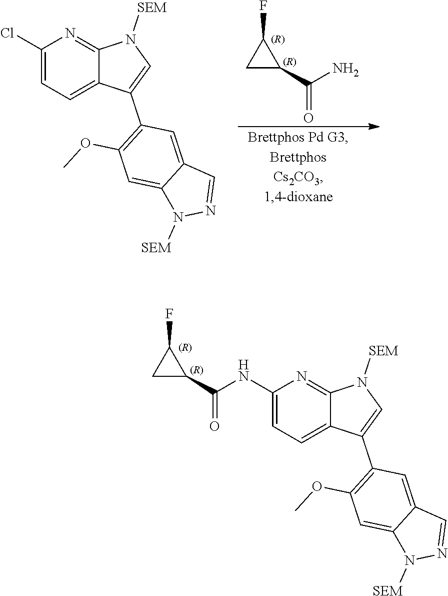

To a solution of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]indazole (250.0 mg, 0.47 mmol) in dioxane (10.0 mL) was added Cs₂CO₃ (436.9 mg, 1.31 mmol), (1R,2R)-2-fluorocyclopropane-1-carboxamide (138.6 mg, 1.34 mmol), Brettphos Pd G3 (81.0 mg, 0.09 mmol) and BrettPhos (23.9 mg, 0.05 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1R,2R)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide (100.0 mg, 36%) as a yellow solid. LCMS (ESI, m/z): [M+H]⁺=626.3.

### Step 5: Synthesis of (1R,2R)-2-fluoro-N-[3-(6-methoxy-1H-indazol-5-yl)-1H-pyrrolo[2,3-b]pyridin-6-yl]cyclopropane-1-carboxamide (Compound 53)

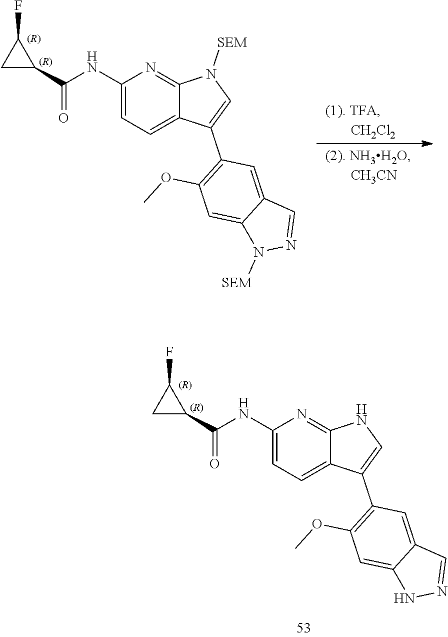

To a solution of (1R,2R)-2-fluoro-N-[3-(6-methoxy-1-[[2-(trimethylsilyl)ethoxy]methyl]indazol-5-yl)-1-[[2-(trimethylsilyl)ethoxy]methyl]pyrrolo[2,3-b]pyridin-6-yl]cyclopropane-1-carboxamide (100.0 mg, 0.16 mmol) in CH₂Cl₂ (5.0 mL) was added TFA (5.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 9% B to 26% B in 8 min; 254 nm) to afford (1R,2R)-2-fluoro-N-[3-(6-methoxy-1H-indazol-5-yl)-1H-pyrrolo[2,3-b]pyridin-6-yl]cyclopropane-1-carboxamide (4.5 mg, 8%) as a white solid. LCMS (ESI, m/z): [M+H]⁺=366.1. ¹H NMR (400 MHz, DMSO-d₆): δ 12.82 (s, 1H), 11.49 (s, 1H), 10.60 (s, 1H), 7.97-7.94 (m, 2H), 7.91-7.85 (m, 1H), 7.80 (s, 1H), 7.51 (d, J=2.4 Hz, 1H), 7.06 (s, 1H), 5.01-4.83 (m, 1H), 3.86 (s, 3H), 2.29-2.18 (m, 1H), 1.69-1.62 (m, 1H), 1.18-1.15 (m, 1H).

### Example S54. Compound 54

### Step 1: Synthesis of (1S,2S)-2-fluoro-N-[3-(2-methoxyphenyl)-1-[[2-(trimethylsilyl)ethoxy]methyl]pyrazolo[3,4-b]pyridin-6-yl]cyclopropane-1-carboxamide

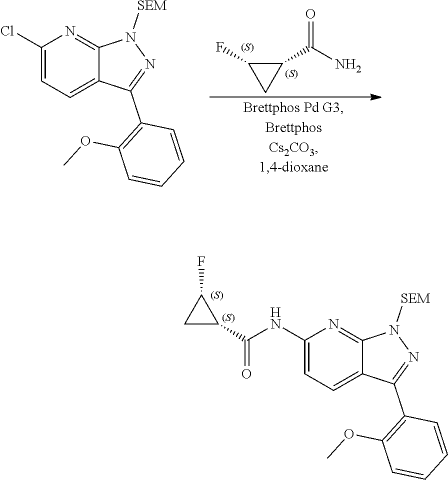

A solution of 6-chloro-3-(2-methoxyphenyl)-1-[[2-(trimethylsilyl)ethoxy]methyl]pyrazolo[3,4-b]pyridine (300.0 mg, 0.77 mmol), (1S,2S)-2-fluorocyclopropane-1-carboxamide (396.6 mg, 3.85 mmol), Cs₂CO₃ (752.0 mg, 2.31 mmol), BrettPhos (82.6 mg, 0.15 mmol) and BrettPhos Pd G3 (69.7 mg, 0.08 mmol) in dioxane (10.0 mL) was irradiated with microwave radiation at 120° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1S,2S)-2-fluoro-N-[3-(2-methoxyphenyl)-1-[[2-(trimethylsilyl)ethoxy]methyl]pyrazolo[3,4-b]pyridin-6-yl]cyclopropane-1-carboxamide (250.0 mg, 71%) as a yellow solid. LCMS (ESI, m/z): [M+H]⁺=457.2.

### Step 2: Synthesis of (1S,2S)-2-fluoro-N-[3-(2-methoxyphenyl)-1H-pyrazolo[3,4-b]pyridin-6-yl]cyclopropane-1-carboxamide (Compound 54)

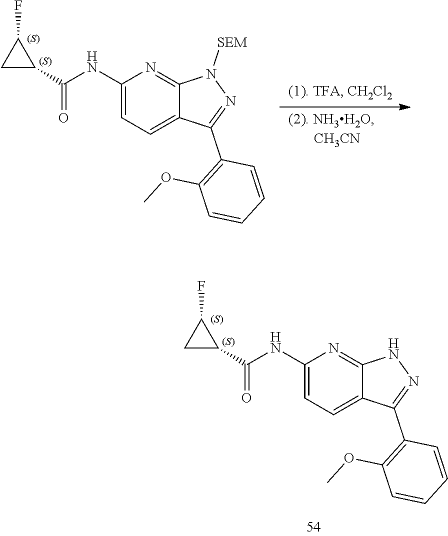

A mixture of (1S,2S)-2-fluoro-N-[3-(2-methoxyphenyl)-1-[[2-(trimethylsilyl)ethoxy]methyl]pyrazolo[3,4-b]pyridin-6-yl]cyclopropane-1-carboxamide (250.0 mg, 0.55 mmol) and TFA (5.0 mL) in CH₂Cl₂ (5.0 mL) was stirred at room temperature for 3 h. The resulting mixture was concentrated under vacuum. The residue was dissolved in NH₃·H₂O (5.0 mL) and ACN (5.0 mL). The mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with Water/ACN (54/46, v/v) and then purified by Prep-HPLC with the following conditions (Column: YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 26% B to 56% B in 10 min; 254 nm) to afford (1S,2S)-2-fluoro-N-[3-(2-methoxyphenyl)-1H-pyrazolo[3,4-b]pyridin-6-yl]cyclopropane-1-carboxamide (82.4 mg, 46%) as a white solid. LCMS (ESI, m/z): [M+H]⁺=327.2 ¹H NMR (300 MHz, DMSO-d₆): δ 13.45 (s, 1H), 10.94 (s, 1H), 8.11 (d, J=9.0 Hz, 1H), 7.98 (d, J=8.7 Hz, 1H), 7.64-7.60 (m, 1H), 7.47-7.41 (m, 1H), 7.19 (d, J=7.8 Hz, 1H), 7.09-7.04 (m, 1H), 5.07-4.83 (m, 1H), 3.83 (s, 3H), 2.29-2.24 (m, 1H), 1.72-1.63 (m, 1H), 1.23-1.11 (m, 1H).

### Example S55. Compound 55

### Step 1: Synthesis of 6-chloro-3-[imidazo[1,2-a]pyridin-7-yl]-1-[[2-(trimethylsilyl)ethoxy]methyl]pyrrolo[2,3-b]pyridine

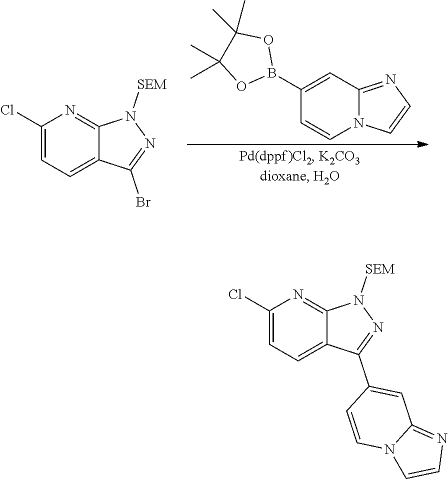 [SMILES: C[Si](C)(C)CCOCn1nc(Br)c2ccc(Cl)nc21 ; CC1(C)OB(c2ccn3ccnc3c2)OC1(C)C ; C[Si](C)(C)CCOCn1nc(-c2ccn3ccnc3c2)c2ccc(Cl)nc21]

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (5.0/1.0 mL) was added 7-(4,4,5,5-tetramethyl-1,3,2-dioxaborolan-2-yl)imidazo[1,2-a]pyridine (337.4 mg, 1.38 mmol), K₂CO₃ (573.1 mg, 4.15 mmol) and Pd(dppf)Cl₂ (101.1 mg, 0.07 mmol). The resulting mixture was stirred at 80° C. for 3 h under N₂. After the reaction was completed, the resulting mixture was concentrated under vacuum. The residue was purified by flash column chromatography with petroleum ether/ethyl acetate (80/20, v/v) to afford 6-chloro-3-[imidazo[1,2-a]pyridin-7-yl]-1-[[2-(trimethylsilyl)ethoxy]methyl]pyrrolo[2,3-b]pyridine (470.0 mg, 85%) as a yellow solid. LCMS (ESI, m/z): [M+H]⁺=399.1.

### Step 2: Synthesis of N-(3-[imidazo[1,2-a]pyridin-7-yl]-1-[[2-(trimethylsilyl)ethoxy]methyl]pyrrolo[2,3-b]pyridin-6-yl)cyclopropanecarboxamide

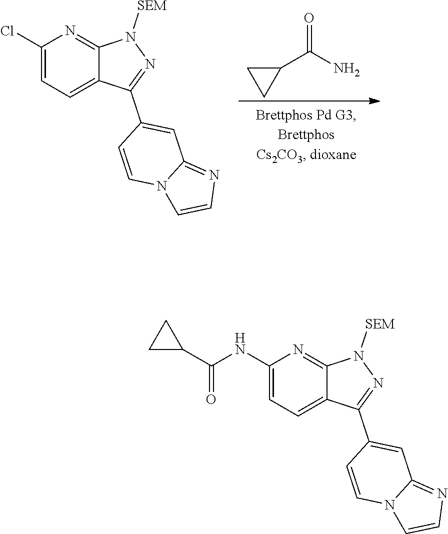 [SMILES: C[Si](C)(C)CCOCn1nc(-c2ccn3ccnc3c2)c2ccc(Cl)nc21 ; NC(=O)C1CC1 ; C[Si](C)(C)CCOCn1nc(-c2ccn3ccnc3c2)c2ccc(NC(=O)C3CC3)nc21]

To a solution of 6-chloro-3-[imidazo[1,2-a]pyridin-7-yl]-1-[[2-(trimethylsilyl)ethoxy]methyl]pyrrolo[2,3-b]pyridine (440.0 mg, 1.10 mmol) in 1,4-dioxane (10.0 mL) was added cyclopropanecarboxamide (469.3 mg, 5.51 mmol), BrettPhos (118.4 mg, 0.22 mmol), Cs₂CO₃ (1.1 g, 3.31 mmol) and BrettPhos Pd G3 (100.0 mg, 0.11 mmol). The resulting mixture was stirred at 100° C. for 2 h under N₂. After the reaction was completed, the resulting mixture was concentrated under vacuum. The residue was purified by flash column chromatography with CH₂Cl₂/MeOH (95/5, v/v) to afford N-(3-[imidazo[1,2-a]pyridin-7-yl]-1-[[2-(trimethylsilyl)ethoxy]methyl]pyrrolo[2,3-b]pyridin-6-yl)cyclopropanecarboxamide (450.0 mg, 91%) as a yellow solid. LCMS (ESI, m/z): [M+H]⁺=448.2.

### Step 3: Synthesis of N-(3-[imidazo[1,2-a]pyridin-7-yl]-1H-pyrrolo[2,3-b]pyridin-6-yl)cyclopropanecarboxamide (Compound 55)

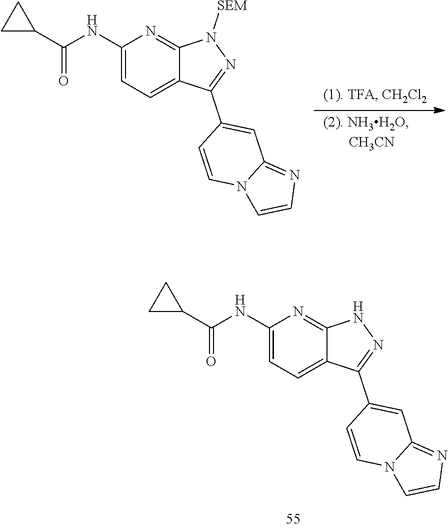 [SMILES: C[Si](C)(C)CCOCn1nc(-c2ccn3ccnc3c2)c2ccc(NC(=O)C3CC3)nc21 ; O=C(Nc1ccc2c(-c3ccn4ccnc4c3)n[nH]c2n1)C1CC1]

To a solution of N-(3-[imidazo[1,2-a]pyridin-7-yl]-1-[[2-(trimethylsilyl)ethoxy]methyl]pyrrolo[2,3-b]pyridin-6-yl)cyclopropanecarboxamide (400.0 mg, 0.89 mmol) in CH₂Cl₂ (3.0 mL) was added TFA (3.0 mL). The mixture was stirred at room temperature for 1 h. The mixture was concentrated under vacuum. The residue was dissolved in ACN (3.0 mL) and NH₃·H₂O (3.0 mL). The resulting mixture was stirred at room temperature for additional 16 h. After the reaction was completed, the resulting mixture was concentrated under vacuum. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 10 min; 254 nm) to afford N-(3-[imidazo[1,2-a]pyridin-7-yl]-1H-pyrrolo[2,3-b]pyridin-6-yl)cyclopropanecarboxamide (16.4 mg, 5%) as a white solid. LCMS (ESI, m/z): [M+H]⁺=318.2. ¹H NMR (400 MHz, DMSO-d₆): δ 11.83 (s, 1H), 10.66 (s, 1H), 8.55 (d, J=7.2 Hz, 1H), 8.40 (d, J=8.8 Hz, 1H), 8.00-7.99 (m, 2H), 7.89 (s, 1H), 7.82 (s, 1H), 7.53 (s, 1H), 7.35-7.33 (m, 1H), 2.08-2.03 (m, 1H), 0.86-0.81 (m, 4H).

### Example S56. Compound 56

### Step 1: Synthesis of 6-chloro-3-[imidazo[1,2-a]pyridin-6-yl]-1-[[2-(trimethylsilyl)ethoxy]methyl]pyrrolo[2,3-b]pyridine

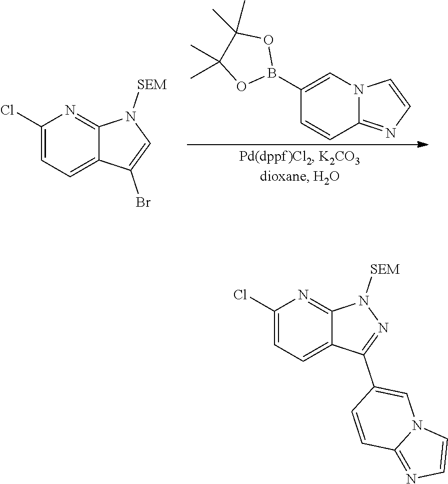 [SMILES: C[Si](C)(C)CCOCn1cc(Br)c2ccc(Cl)nc21 ; CC1(C)OB(c2ccc3nccn3c2)OC1(C)C ; C[Si](C)(C)CCOCn1nc(-c2ccc3nccn3c2)c2ccc(Cl)nc21]

To a solution of 3-bromo-6-chloro-1-[[2-(trimethylsilyl)ethoxy]methyl]pyrrolo[2,3-b]pyridine (500.0 mg, 1.38 mmol) in dioxane/H₂O (15.0/3.0 mL) was added 6-(4,4,5,5-tetramethyl-1,3,2-dioxaborolan-2-yl)imidazo[1,2-a]pyridine (337.4 mg, 1.38 mmol), K₂CO₃ (573.9 mg, 4.17 mmol) and Pd(dppf)Cl₂ (101.4 mg, 0.18 mmol) at room temperature under N₂. The resulting mixture was stirred at 80° C. for 16 h under N₂. After the reaction was completed, the resulting mixture was concentrated under reduced pressure. The residue was purified by flash column chromatography with CH₂Cl₂/MeOH (95/5, v/v) to afford 6-chloro-3-[imidazo[1,2-a]pyridin-6-yl]-1-[[2-(trimethylsilyl)ethoxy]methyl]pyrrolo[2,3-b]pyridine (200.0 mg, 82%) as a yellow solid. LCMS (ESI, m/z): [M+H]⁺=399.1.

### Step 2: Synthesis of N-(3-[imidazo[1,2-a]pyridin-6-yl]-1-[[2-(trimethylsilyl)ethoxy]methyl]pyrrolo[2,3-b]pyridin-6-yl)cyclopropanecarboxamide

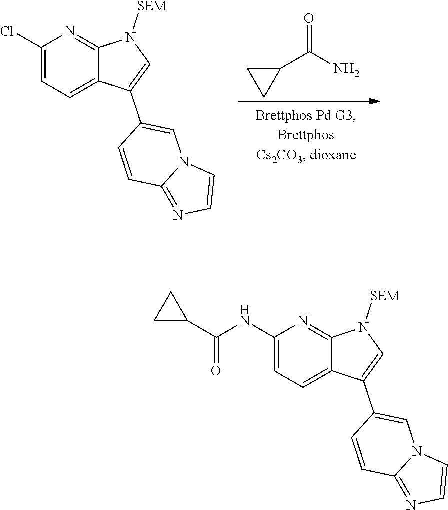 [SMILES: C[Si](C)(C)CCOCn1cc(-c2ccc3nccn3c2)c2ccc(Cl)nc21 ; NC(=O)C1CC1 ; C[Si](C)(C)CCOCn1cc(-c2ccc3nccn3c2)c2ccc(NC(=O)C3CC3)nc21]

To a solution of 6-chloro-3-[imidazo[1,2-a]pyridin-6-yl]-1-[[2-(trimethylsilyl)ethoxy]methyl]pyrrolo[2,3-b]pyridine (430.0 mg, 1.08 mmol) in dioxane (10.0 mL) was added cyclopropanecarboxamide (275.8 mg, 3.23 mmol), BrettPhos (115.7 mg, 0.26 mmol), Brettphos Pd G3 (97.7 mg, 0.32 mmol) and Cs₂CO₃ (1053.8 mg, 3.23 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-(3-[imidazo[1,2-a]pyridin-6-yl]-1-[[2-(trimethylsilyl)ethoxy]methyl]pyrrolo[2,3-b]pyridin-6-yl)cyclopropanecarboxamide (450.0 mg, 93%) as a yellow solid. LCMS (ESI, m/z): [M+H]⁺=448.2.

### Step 3: Synthesis of N-(3-(imidazo[1,2-a]pyridin-6-yl)-1H-pyrrolo[2,3-b]pyridin-6-yl)cyclopropanecarboxamide (Compound 56)

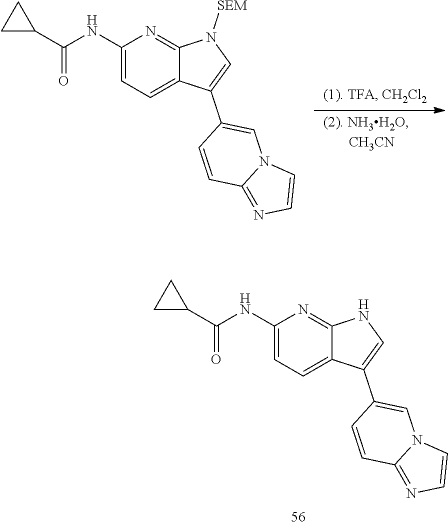 [SMILES: C[Si](C)(C)CCOCn1cc(-c2ccc3nccn3c2)c2ccc(NC(=O)C3CC3)nc21 ; O=C(Nc1ccc2c(-c3ccc4nccn4c3)c[nH]c2n1)C1CC1]

To a solution of N-(3-[imidazo[1,2-a]pyridin-6-yl]-1-[[2-(trimethylsilyl)ethoxy]methyl]pyrrolo[2,3-b]pyridin-6-yl)cyclopropanecarboxamide (400.0 mg, 0.89 mmol) in CH₂Cl₂ (10.0 mL) was added TFA (10.0 mL) at room temperature. The resulting mixture was stirred at room temperature for 4 h. The resulting mixture was concentrated under reduced pressure. The residue was dissolved in CH₃CN (10.0 mL) and NH₃·H₂O (10.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 18% B to 48% B in 8 min; 254 nm) to afford N-(3-(imidazo[1,2-a]pyridin-6-yl)-1H-pyrrolo[2,3-b]pyridin-6-yl)cyclopropanecarboxamide (21.7 mg, 7%) as a white solid. LCMS (ESI, m/z): [M+H]⁺=318.1. ¹H NMR (300 MHz, DMSO-d₆): δ 11.72 (s, 1H), 10.65 (s, 1H), 8.94 (s, 1H), 8.37 (d, J=8.7 Hz, 1H), 7.99-7.97 (m, 2H), 7.84 (d, J=2.7 Hz, 1H), 7.64-7.55 (m, 3H), 2.06-1.99 (m, 1H), 0.84-0.79 (m, 4H).

### Example S57. Compound 57

### Step 1: Synthesis of 5-(6-chloro-1-[[2-(trimethylsilyl)ethoxy]methyl]pyrrolo[2,3-b]pyridin-3-yl)-1-methylpyridin-2-one

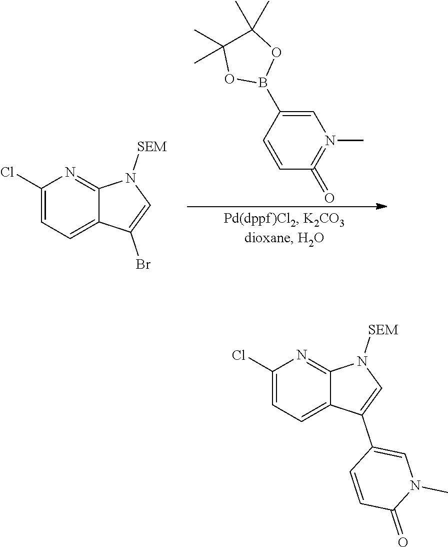

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (5.0/1.0 mL) was added 1-methyl-5-(4,4,5,5-tetramethyl-1,3,2-dioxaborolan-2-yl)pyridin-2-one (121.3 mg, 0.99 mmol), K₂CO₃ (389.9 mg, 1.65 mmol) and Pd(dppf)Cl₂ (121.8 mg, 0.14 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (98/2, v/v) to afford 5-(6-chloro-1-[[2-(trimethylsilyl)ethoxy]methyl]pyrrolo[2,3-b]pyridin-3-yl)-1-methylpyridin-2-one (360.0 mg, 67%) as a white solid. LCMS (ESI, m/z): [M+H]⁺=390.1.

### Step 2: Synthesis of N-[3-(1-methyl-6-oxopyridin-3-yl)-1-[[2-(trimethylsilyl)ethoxy]methyl]pyrrolo[2,3-b]pyridin-6-yl]cyclopropanecarboxamide

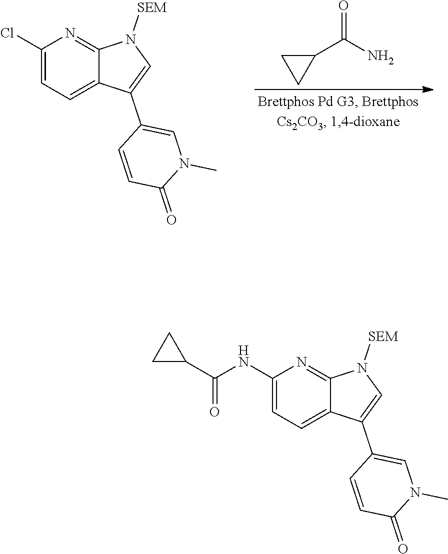 [SMILES: Cn1cc(-c2cn(COCC[Si](C)(C)C)c3nc(Cl)ccc23)ccc1=O ; NC(=O)C1CC1 ; Cn1cc(-c2cn(COCC[Si](C)(C)C)c3nc(NC(=O)C4CC4)ccc23)ccc1=O]

To a solution of 5-(6-chloro-1-[[2-(trimethylsilyl)ethoxy]methyl]pyrrolo[2,3-b]pyridin-3-yl)-1-methylpyridin-2-one (310.0 mg, 0.79 mmol) in dioxane (4.0 mL) was added cyclopropanecarboxamide (338.3 mg, 3.97 mmol), BrettPhos (85.3 mg, 0.16 mmol), Cs₂CO₃ (777.0 mg, 2.38 mmol) and BrettPhos Pd G3 (72.0 mg, 0.08 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (69/31, v/v) to afford N-[3-(1-methyl-6-oxopyridin-3-yl)-1-[[2-(trimethylsilyl)ethoxy]methyl]pyrrolo[2,3-b]pyridin-6-yl]cyclopropanecarboxamide (340.0 mg, 97%) as a white solid. LCMS (ESI, m/z): [M+H]⁺=439.2.

### Step 3: Synthesis of N-[3-(1-methyl-6-oxopyridin-3-yl)-1H-pyrrolo[2,3-b]pyridin-6-yl]cyclopropanecarboxamide (Compound 57)

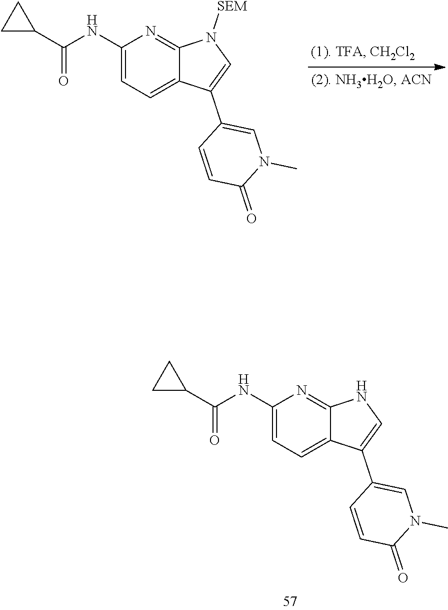 [SMILES: Cn1cc(-c2cn(COCC[Si](C)(C)C)c3nc(NC(=O)C4CC4)ccc23)ccc1=O ; Cn1cc(-c2c[nH]c3nc(NC(=O)C4CC4)ccc23)ccc1=O]

To a solution of N-[3-(1-methyl-6-oxopyridin-3-yl)-1-[[2-(trimethylsilyl)ethoxy]methyl]pyrrolo[2,3-b]pyridin-6-yl]cyclopropanecarboxamide (300.0 mg, 0.68 mmol) in DCM (2.0 mL) was added TFA (2.0 mL) at room temperature. The resulting mixture was stirred at room temperature for 3 h. The resulting mixture was concentrated under reduced pressure. The residue was dissolved in ACN (2.0 mL) and NH₃·H₂O (2.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 19% B to 49% B in 7 min; 254 nm) to afford N-[3-(1-methyl-6-oxopyridin-3-yl)-1H-pyrrolo[2,3-b]pyridin-6-yl]cyclopropanecarboxamide (71.6 mg, 34%) as a white solid. LCMS (ESI, m/z): [M+H]⁺=309.2. ¹H NMR (400 MHz, DMSO-d₆): δ 11.53 (s, 1H), 10.58 (s, 1H), 8.20 (d, J=8.8 Hz, 1H), 7.97 (d, J=2.4 Hz, 1H), 7.90 (d, J=8.8 Hz, 1H), 7.82-7.80 (m, 1H), 7.59 (d, J=2.0 Hz, 1H), 6.46 (d, J=9.6 Hz, 1H), 3.51 (s, 3H), 2.05-1.98 (m, 1H), 0.83-0.77 (m, 4H).

### Example S58. Compound 58

### Step 1: Synthesis of (1S,2S)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide

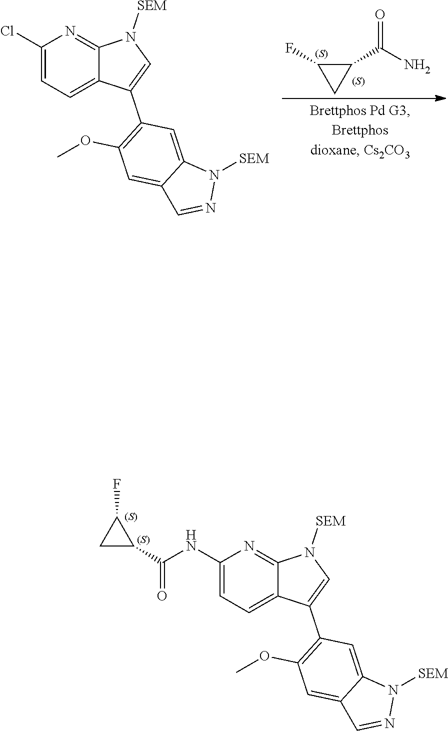

To a solution of 6-(6-chloro-1-[[2-(trimethylsilyl)ethoxy]methyl]pyrrolo[2,3-b]pyridin-3-yl)-5-methoxy-1-[[2-(trimethylsilyl)ethoxy]methyl]indazole (450.0 mg, 0.81 mmol) in 1,4-dioxane (10.0 mL) was added (1S,2S)-2-fluorocyclopropane-1-carboxamide (414.8 mg, 4.02 mmol), BrettPhos (86.4 mg, 0.16 mmol), Cs₂CO₃ (786.5 mg, 2.41 mmol) and BrettPhos Pd G3 (72.9 mg, 0.08 mmol) at room temperature under N₂. The resulting mixture was irradiated with microwave radiation at 120° C. for 1.5 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide (70.0 mg, 14%) as a yellow oil. LCMS (ESI, m/z): [M+H]⁺=626.3.

### Step 2: Synthesis of (1S,2S)-2-fluoro-N-[3-(5-methoxy-1H-indazol-6-yl)-1H-pyrrolo[2,3-b]pyridin-6-yl]cyclopropane-1-carboxamide (Compound 58)

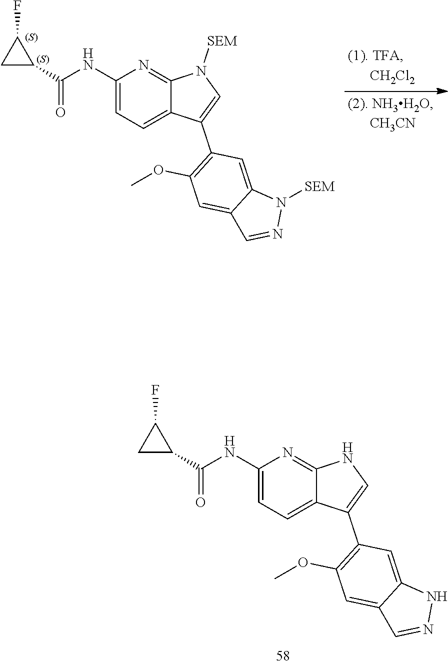

To a solution of (1S,2S)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide (70.0 mg, 0.11 mmol) in DCM (4.0 mL) was added TFA (4.0 mL) at room temperature. The resulting mixture was stirred at room temperature for 2 h. The resulting mixture was concentrated under reduced pressure. The residue was dissolved in CH₃CN (2.0 mL) and NH₃·H₂O (2.0 mL) at room temperature. The resulting mixture was stirred at room temperature for another 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 29% B to 59% B in 7 min; 254 nm) to afford (1S,2S)-2-fluoro-N-[3-(5-methoxy-1H-indazol-6-yl)-1H-pyrrolo[2,3-b]pyridin-6-yl]cyclopropane-1-carboxamide (9.2 mg, 23%) as a white solid. LCMS (ESI, m/z): [M+H]⁺=366.2. ¹H NMR (300 MHz, DMSO-d₆): δ 12.81 (s, 1H), 11.60 (s, 1H), 10.62 (s, 1H), 8.02-7.92 (m, 3H), 7.63-7.61 (m, 2H), 7.30 (s, 1H), 5.14-4.82 (m, 1H), 3.83 (s, 3H), 2.29-2.16 (m, 1H), 1.79-1.52 (m, 1H), 1.34-1.05 (m, 1H).

### Example S59. Compound 59

### Step 1: Synthesis of (1R,2R)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide

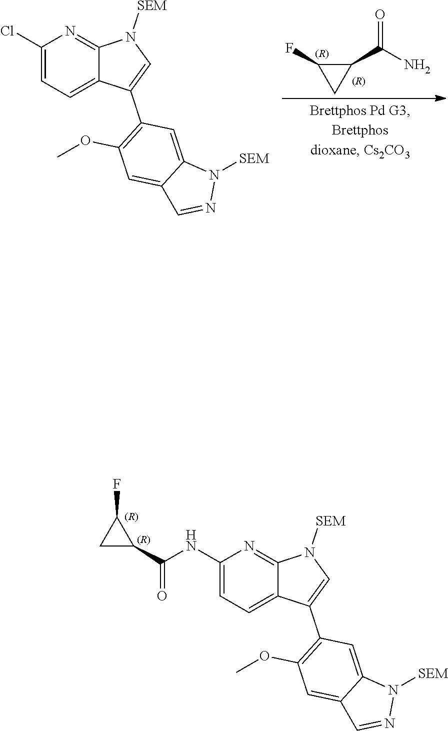

To a solution of 6-(6-chloro-1-[[2-(trimethylsilyl)ethoxy]methyl]pyrrolo[2,3-b]pyridin-3-yl)-5-methoxy-1-[[2-(trimethylsilyl)ethoxy]methyl]indazole (250.0 mg, 0.45 mmol) in 1,4-dioxane (4.0 mL) was added (1R,2R)-2-fluorocyclopropane-1-carboxamide (230.4 mg, 2.24 mmol), BrettPhos (48.0 mg, 0.09 mmol), Cs₂CO₃ (349.6 mg, 1.07 mmol) and BrettPhos Pd G3 (40.5 mg, 0.05 mmol) at room temperature under N₂. The resulting mixture was irradiated with microwave radiation at 120° C. for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with ether/ethyl acetate (3/2, v/v) to afford (1R,2R)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide (200.0 mg, 71%) as a light yellow oil. LCMS (ESI, m/z): [M+H]⁺=626.3.

### Step 2: Synthesis of (1R,2R)-2-fluoro-N-[3-(5-methoxy-1H-indazol-6-yl)-1H-pyrrolo[2,3-b]pyridin-6-yl]cyclopropane-1-carboxamide (Compound 59)

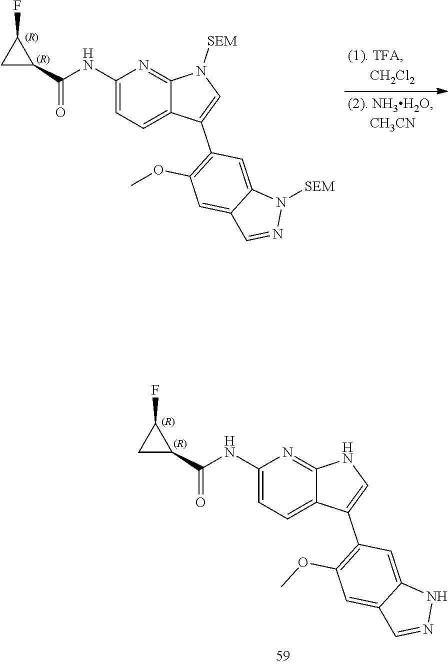

To a solution of (1R,2R)-2-fluoro-N-[3-(5-methoxy-1-[[2-(trimethylsilyl)ethoxy]methyl]indazol-6-yl)-1-[[2-(trimethylsilyl)ethoxy]methyl]pyrrolo[2,3-b]pyridin-6-yl]cyclopropane-1-carboxamide (200.0 mg, 0.32 mmol) in CH₂Cl₂ (3.0 mL) was added TFA (3.0 mL) at room temperature. The resulting mixture was stirred at room temperature for 4 h. After the reaction was completed, the resulting mixture was concentrated under reduced pressure. The residue was dissolved in CH₃CN (3.0 mL) and NH₃·H₂O (3.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 7 min; 254 nm) to afford (1R,2R)-2-fluoro-N-[3-(5-methoxy-1H-indazol-6-yl)-1H-pyrrolo[2,3-b]pyridin-6-yl]cyclopropane-1-carboxamide (30.7 mg, 26%) as a white solid. LCMS (ESI, m/z): [M+H]⁺=366.2. ¹H NMR (300 MHz, DMSO-d₆): δ 12.78 (s, 1H), 11.57 (s, 1H), 10.60 (s, 1H), 7.96-7.84 (m, 3H), 7.57-7.54 (m, 2H), 7.24 (s, 1H), 5.00-4.73 (m, 1H), 3.76 (s, 3H), 2.20-2.15 (m, 1H), 1.66-1.54 (m, 1H), 1.20-1.00 (m, 1H).

### Example S60. Compound 60

### Step 1: Synthesis of 6-chloro-3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridine

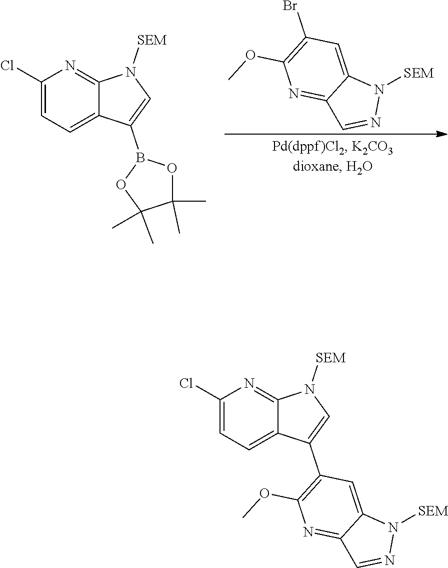 [SMILES: CC1(C)OB(c2cn(COCC[Si](C)(C)C)c3nc(Cl)ccc23)OC1(C)C ; COc1nc2cnn(COCC[Si](C)(C)C)c2cc1Br ; COc1nc2cnn(COCC[Si](C)(C)C)c2cc1-c1cn(COCC[Si](C)(C)C)c2nc(Cl)ccc12]

To a solution of 6-chloro-3-(4,4,5,5-tetramethyl-1,3,2-dioxaborolan-2-yl)-1-[[2-(trimethylsilyl)ethoxy]methyl]pyrrolo[2,3-b]pyridine (400.0 mg, 0.98 mmol) in dioxane (15.0 mL) and H₂O (1.0 mL) was added 6-bromo-5-methoxy-1-[[2-(trimethylsilyl)ethoxy]methyl]pyrazolo[4,3-b]pyridine (140.4 mg, 0.78 mmol), Pd(dppf)Cl₂ (35.8 mg, 0.08 mmol) and K₂CO₃ (202.8 mg, 2.98 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with dichloromethane/methyl alcohol (10/1, v/v) to afford 6-chloro-3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridine (170.0 mg, 58%) as a colorless oil. LCMS (ESI, m/z): [M+H]⁺=560.2.

### Step 2: Synthesis of N-[3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridin-6-yl]cyclopropanecarboxamide

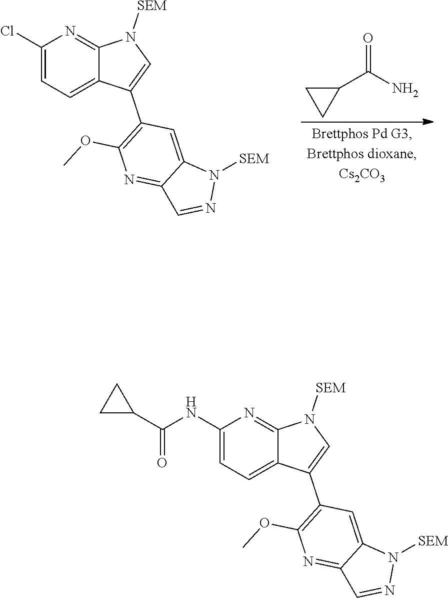 [SMILES: COc1nc2cnn(COCC[Si](C)(C)C)c2cc1-c1cn(COCC[Si](C)(C)C)c2nc(Cl)ccc12 ; NC(=O)C1CC1 ; COc1nc2cnn(COCC[Si](C)(C)C)c2cc1-c1cn(COCC[Si](C)(C)C)c2nc(NC(=O)C3CC3)ccc12]

To a solution of 6-chloro-3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridine (120.0 mg, 0.24 mmol) in dioxane (5.0 mL) was added cyclopropanecarboxamide (54.9 mg, 0.63 mmol), Brettphos Pd G3 (19.4 mg, 0.01 mmol), BrettPhos (22.9 mg, 0.04 mmol) and Cs₂CO₃ (209.7 mg, 0.63 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-[3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridin-6-yl]cyclopropanecarboxamide (100.0 mg, 60%) as a red oil. LCMS (ESI, m/z): [M+H]⁺=609.3.

### Step 3: Synthesis of N-(3-[5-methoxy-1H-pyrazolo[4,3-b]pyridin-6-yl]-1H-pyrrolo[2,3-b]pyridin-6-yl)cyclopropanecarboxamide (Compound 60)

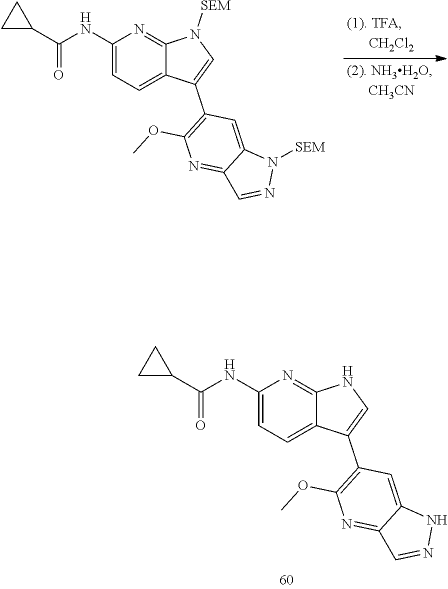 [SMILES: COc1nc2cnn(COCC[Si](C)(C)C)c2cc1-c1cn(COCC[Si](C)(C)C)c2nc(NC(=O)C3CC3)ccc12 ; COc1nc2cn[nH]c2cc1-c1c[nH]c2nc(NC(=O)C3CC3)ccc12]

To a solution of N-[3-(5-methoxy-1-[[2-(trimethylsilyl)ethoxy]methyl]pyrazolo[4,3-b]pyridin-6-yl)-1-[[2-(trimethylsilyl)ethoxy]methyl]pyrrolo[2,3-b]pyridin-6-yl]cyclopropanecarboxamide (100.0 mg, 0.16 mmol) in CH₂Cl₂ (5.0 mL) was added TFA (5.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 7 min; 254 nm) to afford N-(3-[5-methoxy-1H-pyrazolo[4,3-b]pyridin-6-yl]-1H-pyrrolo[2,3-b]pyridin-6-yl)cyclopropanecarboxamide (2.4 mg, 4%) as an off-white solid. LCMS (ESI, m/z): [M+H]⁺=349.1. ¹H NMR (300 MHz, Methanol-d₄): δ 8.17-8.11 (m, 2H), 8.02 (s, 1H), 7.88 (d, J=8.7 Hz, 1H), 7.75 (s, 1H), 4.08 (s, 3H), 1.99-1.89 (m, 1H), 1.03-1.00 (m, 2H), 0.94-0.89 (m, 2H).

### Example S61. Compound 61

### Step 1: Synthesis of 5-bromo-6-methoxy-3-nitropyridin-2-amine

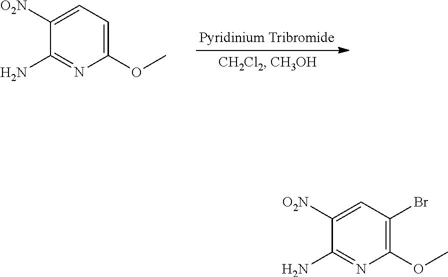

To a solution of 6-methoxy-3-nitropyridin-2-amine (10.0 g, 59.12 mmol) in CH₂Cl₂/MeOH (40.0 mL/10.0 mL) was added pyridinium Tribromide (28.3 g, 88.68 mmol). The resulting mixture was stirred at room temperature for 16 h. After the reaction was completed, the mixture was concentrated under vacuum. The residue was purified by flash column chromatography with petroleum ether/ethyl acetate (70/30, v/v) to afford 5-bromo-6-methoxy-3-nitropyridin-2-amine (7.0 g, 48%) as a yellow solid. LCMS (ESI, m/z): [M+H]⁺=248.0.

### Step 2: Synthesis of 6-bromo-5-methoxy-1H-imidazo[4,5-b]pyridine

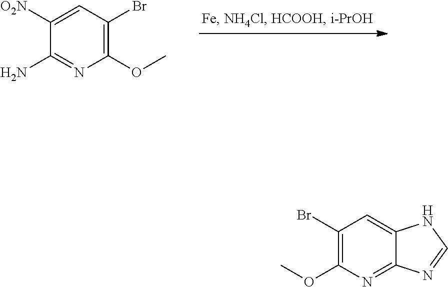

To a solution of 5-bromo-6-methoxy-3-nitropyridin-2-amine (5.0 g, 20.16 mmol) in HCOOH/i-PrOH (50.0 mL/50.0 mL) was added Fe (11.3 g, 201.58 mmol) and NH₄Cl (10.8 g, 201.58 mmol). The resulting mixture was stirred at 80° C. for 5 h. After the reaction was completed, the resulting mixture was filtered. The filtrate was concentrated under vacuum. The residue was purified by flash column chromatography with petroleum ether/ethyl acetate (10/90, v/v) to afford 6-bromo-5-methoxy-1H-imidazo[4,5-b]pyridine (3.0 g, 65%) as a yellow solid. LCMS (ESI, m/z): [M+H]⁺=228.0.

### Step 3: Synthesis of 6-bromo-5-methoxy-1-[[2-(trimethylsilyl)ethoxy]methyl]imidazo[4,5-b]pyridine

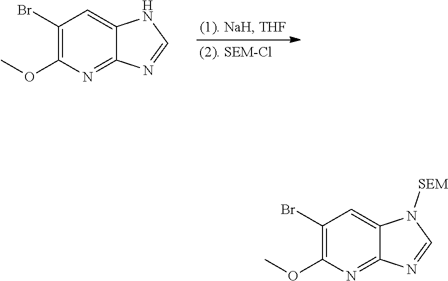 [SMILES: COc1nc2nc[nH]c2cc1Br ; COc1nc2ncn(COCC[Si](C)(C)C)c2cc1Br]

To a solution of 6-bromo-5-methoxy-1H-imidazo[4,5-b]pyridine (3.0 g, 13.16 mmol) in THE (30.0 mL) was added sodium hydride (631.4 mg, 15.79 mmol) at 0° C. under N₂. The mixture was stirred at 0° C. for 30 min. Then SEM-Cl (2.2 g, 13.16 mmol) was added to the mixture at 0° C. under N₂. The resulting mixture was stirred at room temperature for another 2 h. After the reaction was completed, the mixture was quenched with MeOH at 0° C. The resulting mixture was concentrated under vacuum. The residue was purified by flash column chromatography with petroleum ether/ethyl acetate (60/40, v/v) to afford 6-bromo-5-methoxy-1-[[2-(trimethylsilyl)ethoxy]methyl]imidazo[4,5-b]pyridine (1.4 g, 30%) as a yellow solid. LCMS (ESI, m/z): [M+H]⁺=358.1.

### Step 4: Synthesis of 5-methoxy-6-(4,4,5,5-tetramethyl-1,3,2-dioxaborolan-2-yl)-1-[[2-(trimethylsilyl)ethoxy]methyl]imidazo[4,5-b]pyridine

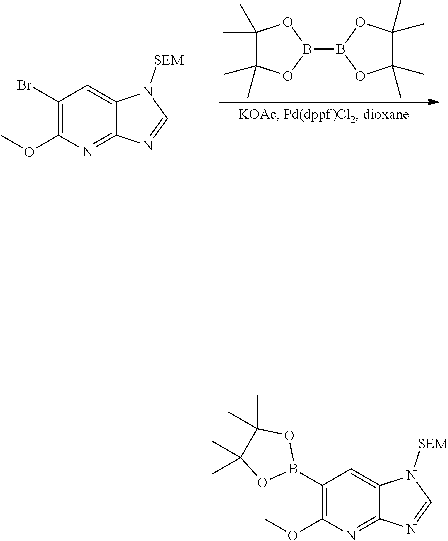

To a solution of 6-bromo-5-methoxy-1-[[2-(trimethylsilyl)ethoxy]methyl]imidazo[4,5-b]pyridine (1.3 g, 3.77 mmol) in 1,4-dioxane (10.0 mL) was added bis(pinacolato)diboron (1.9 g, 7.54 mmol), KOAc (1.1 g, 11.30 mmol) and Pd(dppf)Cl₂ (275.7 mg, 0.38 mmol). The mixture was stirred at 80° C. for 16 h under N₂. After the reaction was completed, the resulting mixture was concentrated under vacuum. The residue was purified by reverse phase flash chromatography with H₂O/ACN (30/70, v/v) to afford 5-methoxy-6-(4,4,5,5-tetramethyl-1,3,2-dioxaborolan-2-yl)-1-[[2-(trimethylsilyl)ethoxy]methyl]imidazo[4,5-b]pyridine (820.0 mg, 53%) as a yellow solid. LCMS (ESI, m/z): [M+H]⁺=406.2.

### Step 5: Synthesis of 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine

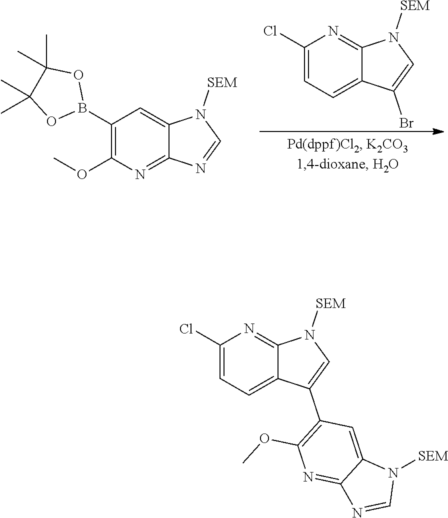 [SMILES: COc1nc2ncn(COCC[Si](C)(C)C)c2cc1B1OC(C)(C)C(C)(C)O1 ; C[Si](C)(C)CCOCn1cc(Br)c2ccc(Cl)nc21 ; COc1nc2ncn(COCC[Si](C)(C)C)c2cc1-c1cn(COCC[Si](C)(C)C)c2nc(Cl)ccc12]

To a solution of 5-methoxy-6-(4,4,5,5-tetramethyl-1,3,2-dioxaborolan-2-yl)-1-[[2-(trimethylsilyl)ethoxy]methyl]imidazo[4,5-b]pyridine (220.0 mg, 0.54 mmol) in 1,4-dioxane/H₂O (3.0 mL/1.0 mL) was added 3-bromo-6-chloro-1-[[2-(trimethylsilyl)ethoxy]methyl]pyrrolo[2,3-b]pyridine (196.32 mg, 0.54 mmol), K₂CO₃ (225.0 mg, 1.63 mmol) and Pd(dppf)Cl₂ (39.7 mg, 0.05 mmol). The mixture was stirred at 80° C. for 16 h under N₂. After the reaction was completed, the resulting mixture was concentrated under vacuum. The residue was purified by flash column chromatography with petroleum ether/ethyl acetate (12/88, v/v) to afford 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine (140.0 mg, 46%) as a yellow solid. LCMS (ESI, m/z): [M+H]⁺=560.2.

### Step 6: Synthesis of N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropanecarboxamide

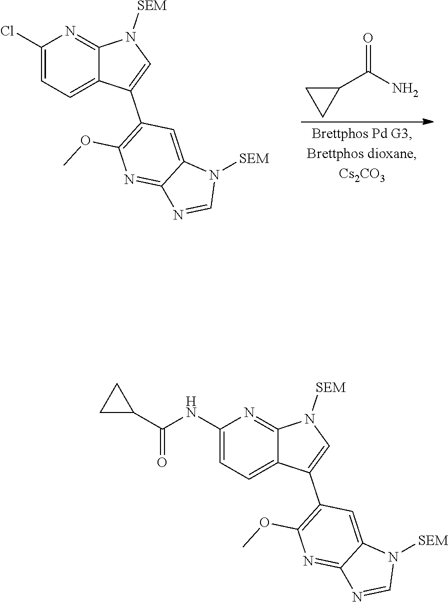 [SMILES: COc1nc2ncn(COCC[Si](C)(C)C)c2cc1-c1cn(COCC[Si](C)(C)C)c2nc(Cl)ccc12 ; NC(=O)C1CC1 ; COc1nc2ncn(COCC[Si](C)(C)C)c2cc1-c1cn(COCC[Si](C)(C)C)c2nc(NC(=O)C3CC3)ccc12]

To a solution of 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine (120.0 mg, 0.21 mmol) in 1,4-dioxane (3.0 mL) was added cyclopropanecarboxamide (54.7 mg, 0.64 mmol), Cs₂CO₃ (209.4 mg, 0.64 mmol), BrettPhos (23.0 mg, 0.043 mmol) and BrettPhos Pd G3 (19.4 mg, 0.02 mmol). The resulting mixture was stirred at 100° C. for 2 h. After the reaction was completed, the resulting mixture was concentrated under vacuum. The residue was purified by flash column chromatography with petroleum ether/ethyl acetate (20/80, v/v) to afford N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropanecarboxamide (100.0 mg, 76%) as a yellow solid. LCMS (ESI, m/z): [M+H]⁺=609.3.

### Step 7: Synthesis of N-(3-[5-methoxy-1H-imidazo[4,5-b]pyridin-6-yl]-1H-pyrrolo[2,3-b]pyridin-6-yl)cyclopropanecarboxamide (Compound 61)

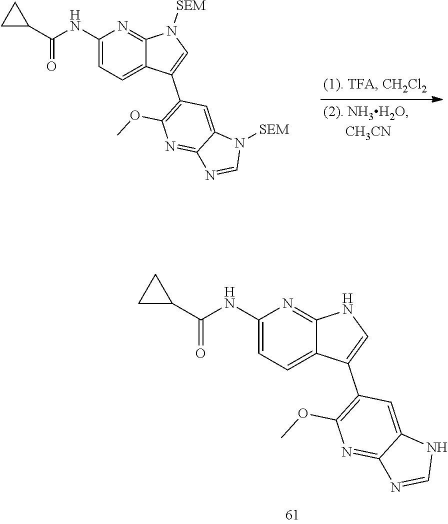 [SMILES: COc1nc2ncn(COCC[Si](C)(C)C)c2cc1-c1cn(COCC[Si](C)(C)C)c2nc(NC(=O)C3CC3)ccc12 ; COc1nc2nc[nH]c2cc1-c1c[nH]c2nc(NC(=O)C3CC3)ccc12]

To a solution of N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropanecarboxamide (80.0 mg, 0.13 mmol) in CH₂Cl₂ (2.0 mL) was added TFA (2.0 mL). The mixture was stirred at room temperature for 2 h. The mixture was concentrated under vacuum. The residue was dissolved in ACN (2.0 mL) and NH₃·H₂O (2.0 mL). The resulting mixture was stirred at room temperature for another 2 h. After the reaction was completed, the resulting mixture was concentrated under vacuum. The residue was purified by Prep-HPLC with the following conditions (Column: YMC-Actus Triart C18 Column, 30×250 mm, 5 um; Mobile Phase A: Water (10 mmol/L NH₄HCO₃), Mobile Phase B: ACN; Flow rate: 60 mL/min; Gradient: 13% B to 43% B in 10 min; 254 nm) to afford N-(3-[5-methoxy-1H-imidazo[4,5-b]pyridin-6-yl]-1H-pyrrolo[2,3-b]pyridin-6-yl)cyclopropanecarboxamide (14.6 mg, 32%) as a light yellow solid. LCMS (ESI, m/z): [M+H]⁺=349.2. ¹H NMR (300 MHz, DMSO-d₆): δ 12.32 (s, 1H), 11.60 (s, 1H), 10.59 (s, 1H), 8.22 (s, 1H), 8.08 (s, 1H), 8.01 (d, J=8.1 Hz, 1H), 7.92 (d, J=8.1 Hz, 1H), 7.63 (s, 1H), 3.96 (s, 3H), 2.09-2.01 (m, 1H), 0.86-0.79 (m, 4H).

### Example S62. Compound 62

### Step 1: Synthesis of 6-chloro-3-(2-ethoxypyridin-3-yl)-1-((2-(trimethylsilyl)ethoxy)methyl)-1H-pyrrolo[2,3-b]pyridine

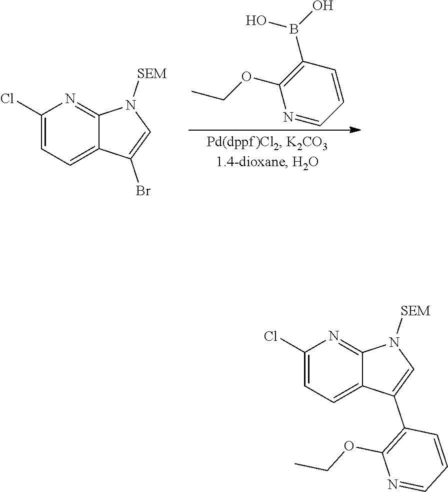 [SMILES: C[Si](C)(C)CCOCn1cc(Br)c2ccc(Cl)nc21 ; CCOc1ncccc1B(O)O ; CCOc1ncccc1-c1cn(COCC[Si](C)(C)C)c2nc(Cl)ccc12]

To a solution of 3-bromo-6-chloro-1-((2-(trimethylsilyl)ethoxy)methyl)-1H-pyrrolo[2,3-b]pyridine (1.0 g, 2.76 mmol) in 1,4-dioxane/H₂O (20.0/4.0 mL) was added (2-ethoxypyridin-3-yl)boronic acid (461.6 mg, 2.76 mmol), K₂CO₃ (1.1 g, 8.29 mmol) and Pd(dppf)Cl₂ (202.3 mg, 0.28 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (96/4, v/v) to afford 6-chloro-3-(2-ethoxypyridin-3-yl)-1-((2-(trimethylsilyl)ethoxy)methyl)-1H-pyrrolo[2,3-b]pyridine (710.0 mg, 63%) as a light yellow oil. LCMS (ESI, m/z): [M+H]⁺=404.1.

### Step 2: Synthesis of (1S,2S)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide

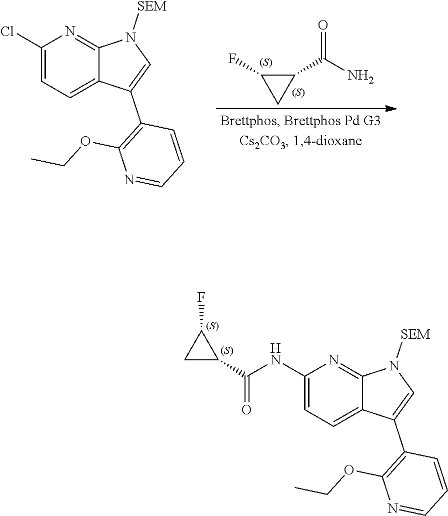

To a solution of 6-chloro-3-(2-ethoxypyridin-3-yl)-1-((2-(trimethylsilyl)ethoxy)methyl)-1H-pyrrolo[2,3-b]pyridine (300.0 mg, 0.74 mmol) in 1,4-dioxane (10.0 mL) was added (1S,2S)-2-fluorocyclopropane-1-carboxamide (382.8 mg, 3.71 mmol), Cs₂CO₃ (725.9 mg, 2.23 mmol), BrettPhos (79.7 mg, 0.15 mmol) and BrettPhos Pd G3 (67.3 mg, 0.07 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford (1S,2S)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide (190.0 mg, 54%) as a yellow oil. LCMS (ESI, m/z): [M+H]⁺=471.2.

### Step 3: Synthesis of (1S,2S)—N-(3-(2-ethoxypyridin-3-yl)-1H-pyrrolo[2,3-b]pyridin-6-yl)-2-fluorocyclopropane-1-carboxamide (Compound 62)

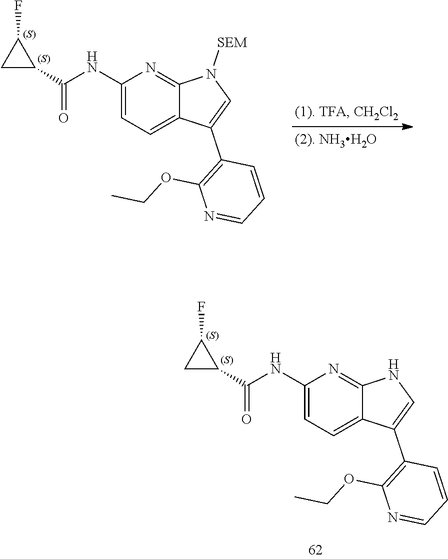

To a solution of (1S,2S)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide (190.0 mg, 0.40 mmol) in CH₂Cl₂ (5.0 mL) was added TFA (5.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 10 min; 254 nm) to afford (1S,2S)—N-(3-(2-ethoxypyridin-3-yl)-1H-pyrrolo[2,3-b]pyridin-6-yl)-2-fluorocyclopropane-1-carboxamide (42.2 mg, 30%) as a white solid. LCMS (ESI, m/z): [M+H]⁺=341.2. ¹H NMR (400 MHz, DMSO-d₆): δ 11.69 (s, 1H), 10.66 (s, 1H), 8.15 (d, J=8.8 Hz, 1H), 8.06-7.92 (m, 3H), 7.75 (s, 1H), 7.07-7.04 (m, 1H), 5.01-4.84 (m, 1H), 4.44-4.39 (m, 2H), 2.29-2.22 (m, 1H), 1.69-1.63 (m, 1H), 1.37-1.34 (m, 3H), 1.20-1.13 (m, 1H).

### Example S63. Compound 63

### Step 1: Synthesis of (1R,2R)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide

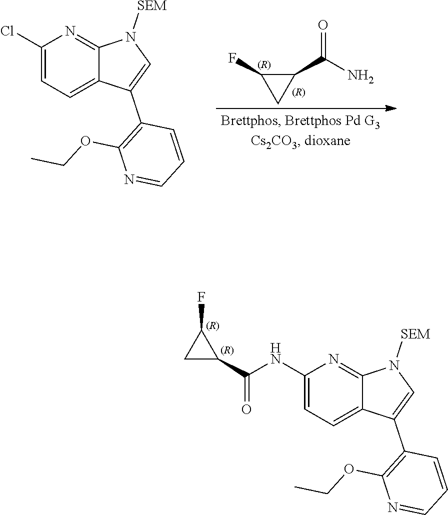

To a solution of 6-chloro-3-(2-ethoxypyridin-3-yl)-1-((2-(trimethylsilyl)ethoxy)methyl)-1H-pyrrolo[2,3-b]pyridine (300.0 mg, 0.74 mmol) in 1,4-dioxane (10.0 mL) was added (1R,2R)-2-fluorocyclopropane-1-carboxamide (382.8 mg, 3.71 mmol), Cs₂CO₃ (725.9 mg, 2.23 mmol), BrettPhos (79.7 mg, 0.15 mmol) and BrettPhos Pd G3 (67.3 mg, 0.07 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford (1R,2R)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide (160.0 mg, 45%) as a white solid. LCMS (ESI, m/z): [M+H]⁺=471.2.

### Step 2: Synthesis of (1R,2R)—N-(3-(2-ethoxypyridin-3-yl)-1H-pyrrolo[2,3-b]pyridin-6-yl)-2-fluorocyclopropane-1-carboxamide (Compound 63)

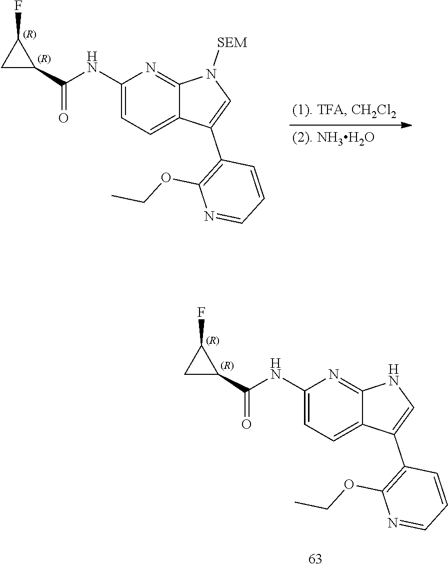

To a solution of (1R,2R)—N-(3-(2-ethoxypyridin-3-yl)-1-((2-(trimethylsilyl)ethoxy)methyl)-1H-pyrrolo[2,3-b]pyridin-6-yl)-2-fluorocyclopropane-1-carboxamide (110.0 mg, 0.23 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL). The resulting mixture was stirred at room temperature for another 2 h. After the reaction was completed, the resulting mixture was concentrated under reduced pressure. The residue was purified by Prep-HPLC with the following conditions (Column: YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 30% B to 60% B in 10 min; 254 nm) to afford (1R,2R)—N-(3-(2-ethoxypyridin-3-yl)-1H-pyrrolo[2,3-b]pyridin-6-yl)-2-fluorocyclopropane-1-carboxamide (31.2 mg, 39%) as a white solid. LCMS (ESI, m/z): [M+H]⁺=341.2. ¹H NMR (300 MHz, DMSO-d₆): δ 11.70 (s, 1H), 10.68 (s, 1H), 8.15 (d, J=8.7 Hz, 1H), 8.07-8.05 (m, 1H), 8.01-7.98 (m, 1H), 7.95-7.92 (m, 1H), 7.75 (s, 1H), 7.08-7.04 (m, 1H), 5.06-4.81 (m, 1H), 4.45-4.38 (m, 2H), 2.26-2.22 (m, 1H), 1.70-1.59 (m, 1H), 1.38-1.33 (m, 3H), 1.20-1.13 (m, 1H).

### Example S64. Compound 64

### Step 1: Synthesis of 3-(6-chloro-1-[[2-(trimethylsilyl)ethoxy]methyl]pyrazolo[3,4-b]pyridin-3-yl)-2-ethoxypyridine

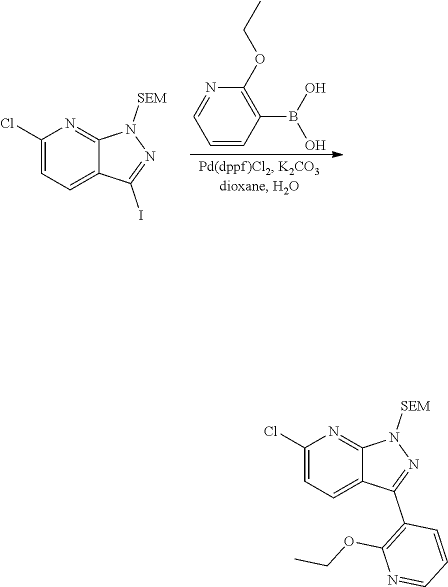 [SMILES: C[Si](C)(C)CCOCn1nc(I)c2ccc(Cl)nc21 ; CCOc1ncccc1B(O)O ; CCOc1ncccc1-c1nn(COCC[Si](C)(C)C)c2nc(Cl)ccc12]

A mixture of 6-chloro-3-iodo-1-[[2-(trimethylsilyl)ethoxy]methyl]pyrazolo[3,4-b]pyridine (1.4 g, 3.42 mmol), 2-ethoxypyridin-3-ylboronic acid (0.6 g, 3.42 mmol), K₂CO₃ (1.4 g, 10.25 mmol) and Pd(dppf)Cl₂ (0.3 g, 0.34 mmol) in dioxane (20.0 mL) and H₂O (4.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (92/8, v/v) to afford 3-(6-chloro-1-[[2-(trimethylsilyl)ethoxy]methyl]pyrazolo[3,4-b]pyridin-3-yl)-2-ethoxypyridine (960.0 mg, 69%) as a yellow oil. LCMS (ESI, m/z): [M+H]⁺=405.1.

### Step 2: Synthesis of (1S,2S)—N-[3-(2-ethoxypyridin-3-yl)-1-[[2-(trimethylsilyl)ethoxy]methyl]pyrazolo[3,4-b]pyridin-6-yl]-2-fluorocyclopropane-1-carboxamide

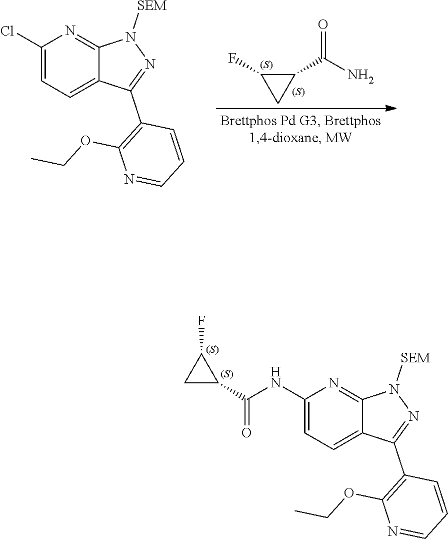

A mixture of 3-(6-chloro-1-[[2-(trimethylsilyl)ethoxy]methyl]pyrazolo[3,4-b]pyridin-3-yl)-2-ethoxypyridine (300.0 mg, 0.74 mmol), (1S,2S)-2-fluorocyclopropane-1-carboxamide (381.9 mg, 3.70 mmol), K₂CO₃ (307.2 mg, 2.22 mmol), BrettPhos (79.5 mg, 0.15 mmol) and BrettPhos Pd G3 (67.15 mg, 0.07 mmol) in dioxane (15.0 mL) was irradiated with microwave radiation (MW) at 120° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1S,2S)—N-[3-(2-ethoxypyridin-3-yl)-1-[[2-(trimethylsilyl)ethoxy]methyl]pyrazolo[3,4-b]pyridin-6-yl]-2-fluorocyclopropane-1-carboxamide (330.0 mg, 94%) as a yellow oil. LCMS (ESI, m/z): [M+H]⁺=472.2.

### Step 3: Synthesis of (1S,2S)—N-[3-(2-ethoxypyridin-3-yl)-1H-pyrazolo[3,4-b]pyridin-6-yl]-2-fluorocyclopropane-1-carboxamide (Compound 64)

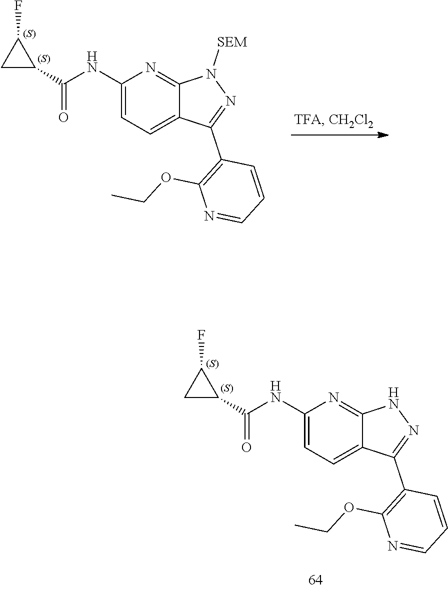

A mixture of (1S,2S)—N-[3-(2-ethoxypyridin-3-yl)-1-[[2-(trimethylsilyl)ethoxy]methyl]pyrazolo[3,4-b]pyridin-6-yl]-2-fluorocyclopropane-1-carboxamide (330.0 mg, 0.70 mmol) and TFA (5.0 mL) in CH₂Cl₂ (5.0 mL) was stirred at room temperature for 16. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: XBridge Shield RP18 OBD Column, 19×250 mm, 10 um; Mobile Phase A: Water (0.1% FA), Mobile Phase B: MeOH—Preparative; Flow rate: 25 mL/min; Gradient: 50% B to 58% B in 10 min; 220/254 nm) to afford (1S,2S)—N-[3-(2-ethoxypyridin-3-yl)-1H-pyrazolo[3,4-b]pyridin-6-yl]-2-fluorocyclopropane-1-carboxamide (3.7 mg, 1%) as a white solid. LCMS (ESI, m/z): [M+H]⁺=342.2. ¹H NMR (300 MHz, DMSO-d₆): δ 13.59 (s, 1H), 10.97 (s, 1H), 8.28-8.23 (m, 2H), 8.06-8.00 (m, 2H), 7.14-7.10 (m, 1H), 5.07-4.83 (m, 1H), 4.48-4.41 (m, 2H), 2.30-2.25 (m, 1H), 1.73-1.63 (m, 1H), 1.32-1.21 (m, 4H).

### Example S65. Compound 65

### Step 1: Synthesis of 3-bromo-N,N-dimethylpyridin-2-amine

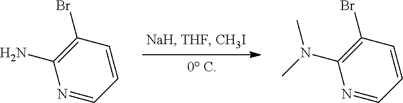

To a solution of 3-bromopyridin-2-amine (5.0 g, 28.90 mmol) in THF (40.0 mL) was added NaH (5.8 g, 60%) at 0° C. under N₂. The resulting mixture was stirred at 0° C. for 1 h. Then CH₃I (8.2 g, 57.80 mmol) was added dropwise to the mixture at 0° C. under N₂. The resulting mixture was stirred at 0° C. for another 1 h. After the reaction was completed, the reaction mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (4/1, v/v) to afford 3-bromo-N,N-dimethylpyridin-2-amine (4.6 g, 79%) as a light yellow oil. LCMS (ESI, m/z): [M+H]⁺=201.0.

### Step 2: Synthesis of 3-(6-chloro-1-((2-(trimethylsilyl)ethoxy)methyl)-1H-pyrrolo[2,3-b]pyridin-3-yl)-N,N-dimethylpyridin-2-amine

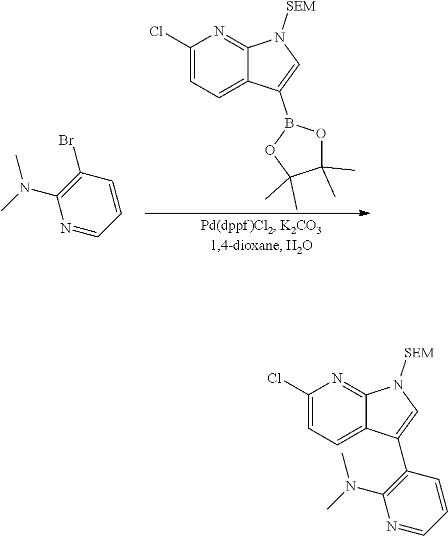

To a solution of 3-bromo-N,N-dimethylpyridin-2-amine (123.0 mg, 0.61 mmol) in dioxane/H₂O (16.0/4.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (250.0 mg, 0.61 mmol), K₂CO₃ (253.6 mg, 1.84 mmol) and Pd(dppf)Cl₂ (44.8 mg, 0.06 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 3-(6-chloro-1-((2-(trimethylsilyl)ethoxy)methyl)-1H-pyrrolo[2,3-b]pyridin-3-yl)-N,N-dimethylpyridin-2-amine (150.0 mg, 60%) as a brown oil. LCMS (ESI, m/z): [M+H]⁺=403.2.

### Step 3: Synthesis of N-(3-(2-(dimethylamino)pyridin-3-yl)-1-((2-(trimethylsilyl)ethoxy)methyl)-1H-pyrrolo[2,3-b]pyridin-6-yl)cyclopropanecarboxamide

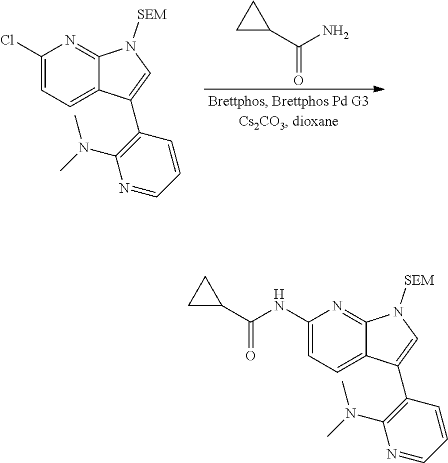 [SMILES: CN(C)c1ncccc1-c1cn(COCC[Si](C)(C)C)c2nc(Cl)ccc12 ; NC(=O)C1CC1 ; CN(C)c1ncccc1-c1cn(COCC[Si](C)(C)C)c2nc(NC(=O)C3CC3)ccc12]

To a solution of 3-(6-chloro-1-((2-(trimethylsilyl)ethoxy)methyl)-1H-pyrrolo[2,3-b]pyridin-3-yl)-N,N-dimethylpyridin-2-amine (130.0 mg, 0.32 mmol) in dioxane (4.0 mL) was added cyclopropanecarboxamide (164.7 mg, 1.94 mmol), Cs₂CO₃ (315.3 mg, 0.97 mmol), Brettphos (34.6 mg, 0.07 mmol) and BrettPhos Pd G3 (29.2 mg, 0.03 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford N-(3-(2-(dimethylamino)pyridin-3-yl)-1-((2-(trimethylsilyl)ethoxy)methyl)-1H-pyrrolo[2,3-b]pyridin-6-yl)cyclopropanecarboxamide (120.0 mg, 82%) as a white solid. LCMS (ESI, m/z): [M+H]⁺=452.2.

### Step 4: Synthesis of N-(3-(2-(dimethylamino)pyridin-3-yl)-1H-pyrrolo[2,3-b]pyridin-6-yl)cyclopropanecarboxamide (Compound 65)

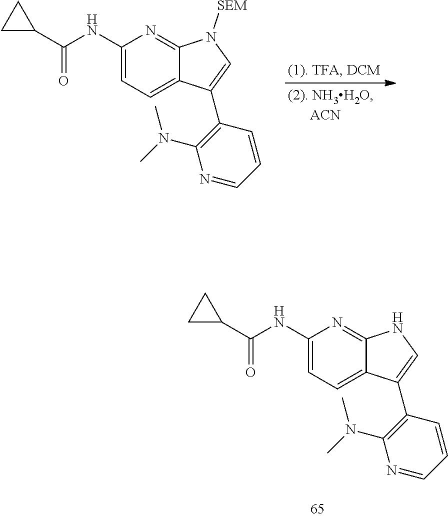 [SMILES: CN(C)c1ncccc1-c1cn(COCC[Si](C)(C)C)c2nc(NC(=O)C3CC3)ccc12 ; CN(C)c1ncccc1-c1c[nH]c2nc(NC(=O)C3CC3)ccc12]

To a solution of N-(3-(2-(dimethylamino)pyridin-3-yl)-1-((2-(trimethylsilyl)ethoxy)methyl)-1H-pyrrolo[2,3-b]pyridin-6-yl)cyclopropanecarboxamide (100.0 mg, 0.22 mmol) in DCM (5.0 mL) was added TFA (5.0 mL) at room temperature. The resulting mixture was stirred at room temperature for 3 h. The resulting mixture was concentrated under reduced pressure. The residue was re-dissolved in ACN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 2% to 21% in 7 min; 254 nm) to afford N-(3-(2-(dimethylamino)pyridin-3-yl)-1H-pyrrolo[2,3-b]pyridin-6-yl)cyclopropanecarboxamide (32.0 mg, 44%) as a light yellow solid. LCMS (ESI, m/z): [M+H]⁺=322.1. ¹H NMR (400 MHz, CD₃OD): δ 8.09-8.08 (m, 1H), 7.90 (d, J=8.8 Hz, 1H), 7.78 (d, J=8.4 Hz, 1H), 7.71-7.68 (m, 1H), 7.47 (s, 1H), 6.94-6.91 (m, 1H), 2.72 (s, 6H), 1.91-1.89 (m, 1H), 1.02-0.99 (m, 2H), 0.92-0.88 (m, 2H).

### Example S66. Compound 66

### Step 1: Synthesis of 6-chloro-3-(2-fluoro-6-methoxyphenyl)-1-[[2-(trimethylsilyl)ethoxy]methyl]pyrazolo[3,4-b]pyridine

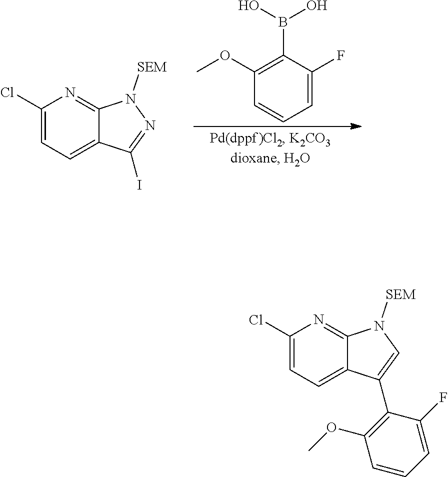 [SMILES: C[Si](C)(C)CCOCn1nc(I)c2ccc(Cl)nc21 ; COc1cccc(F)c1B(O)O ; COc1cccc(F)c1-c1cn(COCC[Si](C)(C)C)c2nc(Cl)ccc12]

A mixture of 6-chloro-3-iodo-1-[[2-(trimethylsilyl)ethoxy]methyl]pyrazolo[3,4-b]pyridine (800.0 mg, 1.95 mmol), 2-fluoro-6-methoxyphenylboronic acid (331.8 mg, 1.95 mmol), K₂CO₃ (809.5 mg, 5.86 mmol) and Pd(dppf)Cl₂ (142.9 mg, 0.20 mmol) in dioxane (20.0 mL) and H₂O (2.0 mL) was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (95/5, v/v) to afford 6-chloro-3-(2-fluoro-6-methoxyphenyl)-1-[[2-(trimethylsilyl)ethoxy]methyl]pyrazolo[3,4-b]pyridine (500.0 mg, 63%) as a yellow oil. LCMS (ESI, m/z): [M+H]⁺=408.1.

### Step 2: Synthesis of (1S,2S)-2-fluoro-N-[3-(2-fluoro-6-methoxyphenyl)-1-[[2-(trimethylsilyl)ethoxy]methyl]pyrazolo[3,4-b]pyridin-6-yl]cyclopropane-1-carboxamide

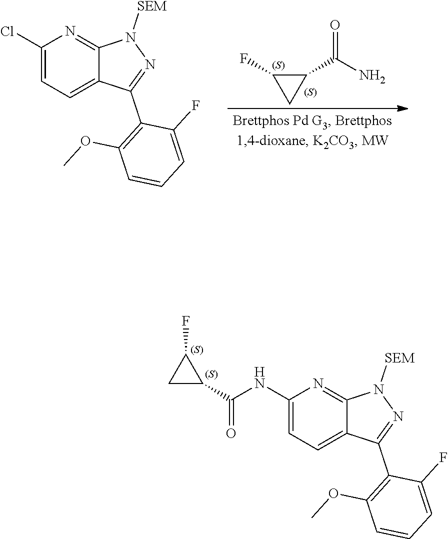

A mixture of 6-chloro-3-(2-fluoro-6-methoxyphenyl)-1-[[2-(trimethylsilyl)ethoxy]methyl]pyrazolo[3,4-b]pyridine (500.0 mg, 1.23 mmol), (1S,2S)-2-fluorocyclopropane-1-carboxamide (631.8 mg, 6.13 mmol), K₂CO₃ (508.2 mg, 3.68 mmol), BrettPhos (131.6 mg, 0.25 mmol) and BrettPhos Pd G3 (111.1 mg, 0.12 mmol) in dioxane (12.0 mL) was irradiated with microwave radiation (MW) at 120° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1S,2S)-2-fluoro-N-[3-(2-fluoro-6-methoxyphenyl)-1-[[2-(trimethylsilyl)ethoxy]methyl]pyrazolo[3,4-b]pyridin-6-yl]cyclopropane-1-carboxamide (254.0 mg, 44%) as a yellow solid. LCMS (ESI, m/z): [M+H]⁺=475.2.

### Step 3: Synthesis of (1S,2S)-2-fluoro-N-[3-(2-fluoro-6-methoxyphenyl)-1H-pyrazolo[3,4-b]pyridin-6-yl]cyclopropane-1-carboxamide (Compound 66)

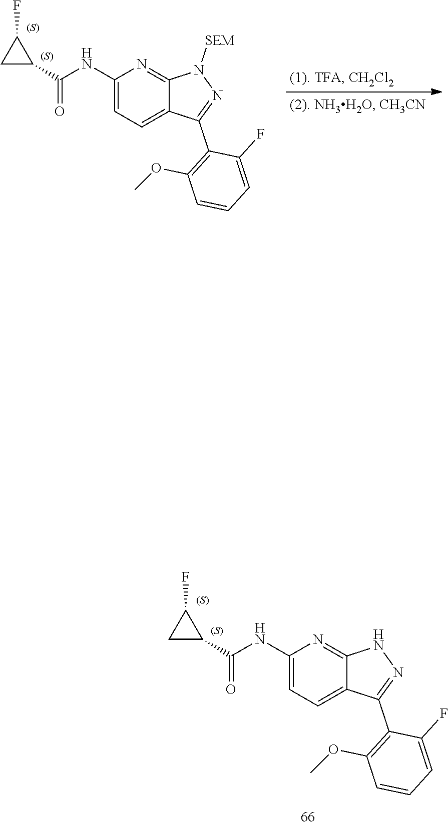

A mixture of (1S,2S)-2-fluoro-N-[3-(2-fluoro-6-methoxyphenyl)-1-[[2-(trimethylsilyl)ethoxy]methyl]pyrazolo[3,4-b]pyridin-6-yl]cyclopropane-1-carboxamide (200.0 mg, 0.42 mmol) and TFA (5.0 mL) in CH₂Cl₂ (5.0 mL) was stirred at room temperature for 2 h. The resulting mixture was concentrated under reduced pressure. The residue was dissolved in NH₃·H₂O (5.0 mL) and CAN (5.0 mL). The resulting mixture was stirred at room temperature for additional 1 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 25% B to 39% B in 10 min; 254/220 nm) to afford (1S,2S)-2-fluoro-N-[3-(2-fluoro-6-methoxyphenyl)-1H-pyrazolo[3,4-b]pyridin-6-yl]cyclopropane-1-carboxamide (24.1 mg, 17%). LCMS (ESI, m/z): [M+H]⁺=345.1. ¹H NMR (300 MHz, DMSO-d₆): δ 13.54 (s, 1H), 10.97 (s, 1H), 8.00-7.89 (m, 2H), 7.50-7.47 (m, 1H), 7.05-6.93 (m, 2H), 5.05-4.81 (m, 1H), 3.76 (s, 3H), 2.29-2.24 (m, 1H), 1.77-1.63 (m, 1H), 1.22-1.17 (m, 1H).

### Example S67. Compound 67

### Step 1: Synthesis of 6-chloro-3-(2-ethoxy-6-fluorophenyl)-1-((2-(trimethylsilyl)ethoxy)methyl)-1H-pyrazolo[3,4-b]pyridine

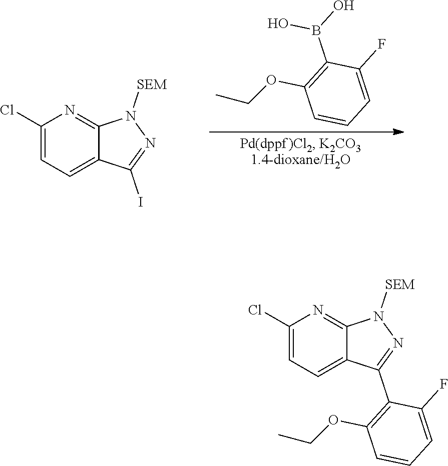

To a solution of 6-chloro-3-iodo-1-((2-(trimethylsilyl)ethoxy)methyl)-1H-pyrazolo[3,4-b]pyridine (500.0 mg, 1.22 mmol) in 1,4-dioxane/H₂O (10.0/2.0 mL) was added (2-ethoxy-6-fluorophenyl)boronic acid (224.5 mg, 1.22 mmol), K₂CO₃ (505.9 mg, 3.66 mmol) and Pd(dppf)Cl₂ (89.2 mg, 0.12 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford 6-chloro-3-(2-ethoxy-6-fluorophenyl)-1-((2-(trimethylsilyl)ethoxy)methyl)-1H-pyrazolo[3,4-b]pyridine (220.0 mg, 42%) as a light yellow oil. LCMS (ESI, m/z): [M+H]⁺=422.1.

### Step 2: Synthesis of (1S,2S)—N-(3-(2-ethoxy-6-fluorophenyl)-1-((2-(trimethylsilyl)ethoxy)methyl)-1H-pyrazolo[3,4-b]pyridin-6-yl)-2-fluorocyclopropane-1-carboxamide

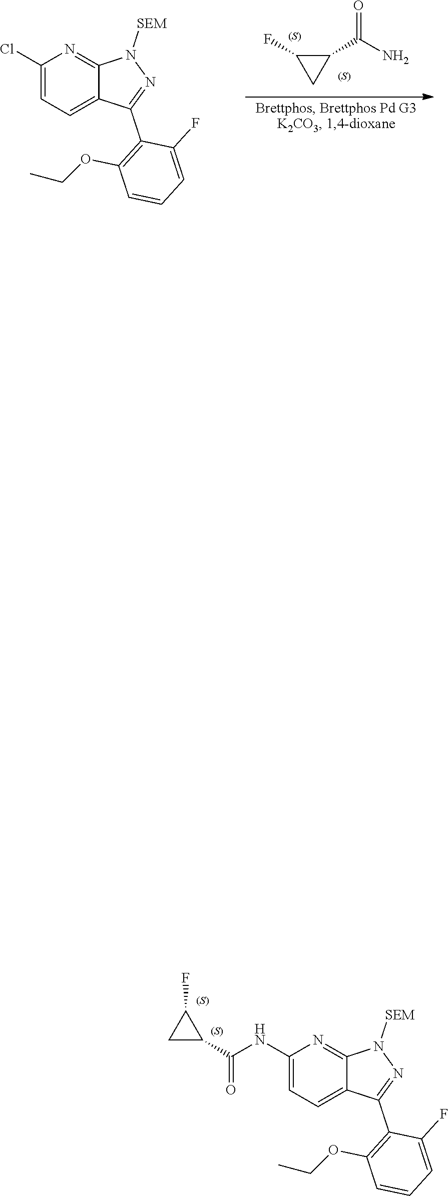

To a solution of 6-chloro-3-(2-ethoxy-6-fluorophenyl)-1-((2-(trimethylsilyl)ethoxy)methyl)-1H-pyrazolo[3,4-b]pyridine (200.0 mg, 0.47 mmol) in 1,4-dioxane (6.0 mL) was added (1S,2S)-2-fluorocyclopropane-1-carboxamide (244.3 mg, 2.37 mmol), K₂CO₃ (196.5 mg, 1.42 mmol), BrettPhos (50.9 mg, 0.10 mmol) and BrettPhos Pd G3 (43.0 mg, 0.05 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (75/25, v/v) to afford (1S,2S)—N-(3-(2-ethoxy-6-fluorophenyl)-1-((2-(trimethylsilyl)ethoxy)methyl)-1H-pyrazolo[3,4-b]pyridin-6-yl)-2-fluorocyclopropane-1-carboxamide (140.0 mg, 60%) as a yellow oil. LCMS (ESI, m/z): [M+H]⁺=489.2.

### Step 3: Synthesis of (1S,2S)—N-(3-(2-ethoxy-6-fluorophenyl)-1H-pyrazolo[3,4-b]pyridin-6-yl)-2-fluorocyclopropane-1-carboxamide (Compound 67)

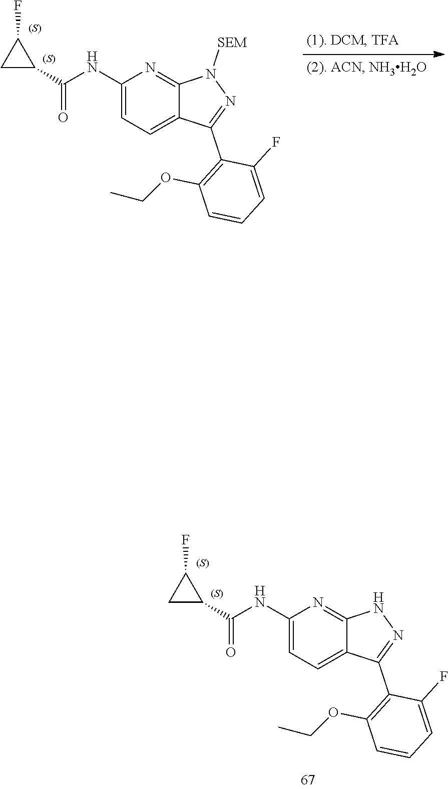

To a solution of (1S,2S)—N-(3-(2-ethoxy-6-fluorophenyl)-1-((2-(trimethylsilyl)ethoxy)methyl)-1H-pyrazolo[3,4-b]pyridin-6-yl)-2-fluorocyclopropane-1-carboxamide (140.0 mg, 0.29 mmol) in DCM (5.0 mL) was added TFA (5.0 mL) at room temperature. The resulting mixture was stirred at room temperature for 4 h. The resulting mixture was concentrated under reduced pressure. The residue was dissolved in ACN (5.0 mL) and NH₃·H₂O (5.0 mL). The resulting mixture was stirred at room temperature for another 2 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 30% B to 60% B in 10 min; 254 nm) to afford (1S,2S)—N-(3-(2-ethoxy-6-fluorophenyl)-1H-pyrazolo[3,4-b]pyridin-6-yl)-2-fluorocyclopropane-1-carboxamide (26.4 mg, 25%) as a light yellow solid. LCMS (ESI, m/z): [M+H]⁺=359.1. ¹H NMR (400 MHz, DMSO-d₆): δ 13.55 (s, 1H), 11.00 (s, 1H), 8.00-7.95 (m, 2H), 7.49-7.43 (m, 1H), 7.03-6.92 (m, 2H), 5.06-4.85 (m, 1H), 4.11-4.05 (m, 2H), 2.29-2.25 (m, 1H), 1.71-1.63 (m, 1H), 1.23-1.12 (m, 4H).

### Example S68. Compound 68

### Step 1: Synthesis of 3-bromo-6-chloro-2-fluoro-1-[[2-(trimethylsilyl)ethoxy]methyl]pyrrolo[2,3-b]pyridine

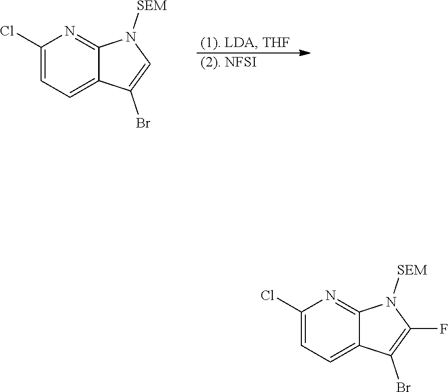 [SMILES: C[Si](C)(C)CCOCn1cc(Br)c2ccc(Cl)nc21 ; "(1). LDA, THF" ; "(2). NFSI" ; C[Si](C)(C)CCOCn1c(F)c(Br)c2ccc(Cl)nc21]

To a solution of 3-bromo-6-chloro-1-[[2-(trimethylsilyl)ethoxy]methyl]pyrrolo[2,3-b]pyridine (1.0 g, 2.78 mmol) in THE (10.0 mL) was added LDA (4.4 mL, 2 mol/L) at −70° C. under N₂. The mixture was stirred at −70° C. for 2 h. Then a solution of NFSI (2.3 g, 7.14 mmol) in THF (8.0 mL) was added dropwise to the mixture at −70° C. The mixture was stirred at −70° C. for another 3 h. After the reaction was completed, the mixture was quenched with aq·NH₄Cl and extracted with ethyl acetate. The combined organic layer was washed with brine, dried over anhydrous sodium sulfate and filtered. The filtrate was concentrated under vacuum. The residue was purified by flash column chromatography with petroleum ether/CH₂Cl₂ (76/24, v/v) to afford 3-bromo-6-chloro-2-fluoro-1-[[2-(trimethylsilyl)ethoxy]methyl]pyrrolo[2,3-b]pyridine (550.0 mg, 80%) as a white solid. LCMS (ESI, m/z): [M+H]⁺=379.0.

### Step 2: Synthesis of 3-(6-chloro-2-fluoro-1-[[2-(trimethylsilyl)ethoxy]methyl]pyrrolo[2,3-b]pyridin-3-yl)-2-methoxypyridine

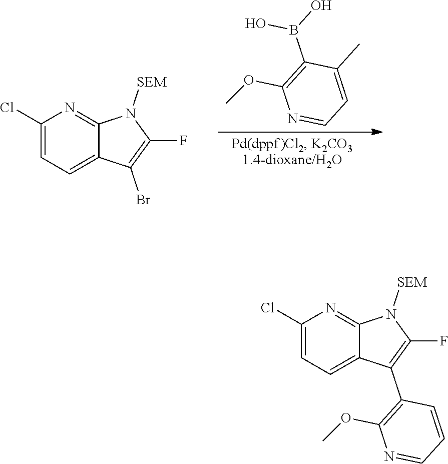

To a solution of 3-bromo-6-chloro-2-fluoro-1-[[2-(trimethylsilyl)ethoxy]methyl]pyrrolo[2,3-b]pyridine (530.0 mg, 1.40 mmol) in 1,4-dioxane/H₂O (5.0 mL/1.0 mL) was added 2-methoxypyridin-3-ylboronic acid (213.5 mg, 1.40 mmol), K₂CO₃ (578.7 mg, 4.19 mmol) and Pd(dppf)Cl₂ (102.1 mg, 0.14 mmol). The resulting mixture was stirred at 80° C. for 16 h under N₂. After the reaction was completed, the mixture was concentrated under vacuum. The residue was purified by flash column chromatography with petroleum ether/ethyl acetate (80/20, v/v) to afford 3-(6-chloro-2-fluoro-1-[[2-(trimethylsilyl)ethoxy]methyl]pyrrolo[2,3-b]pyridin-3-yl)-2-methoxypyridine (300.0 mg, 52%) as a yellow solid. LCMS (ESI, m/z): [M+H]⁺=408.1.

### Step 3: Synthesis of (1S,2S)-2-fluoro-N-[2-fluoro-3-(2-methoxypyridin-3-yl)-1-[[2-(trimethylsilyl)ethoxy]methyl]pyrrolo[2,3-b]pyridin-6-yl]cyclopropane-1-carboxamide

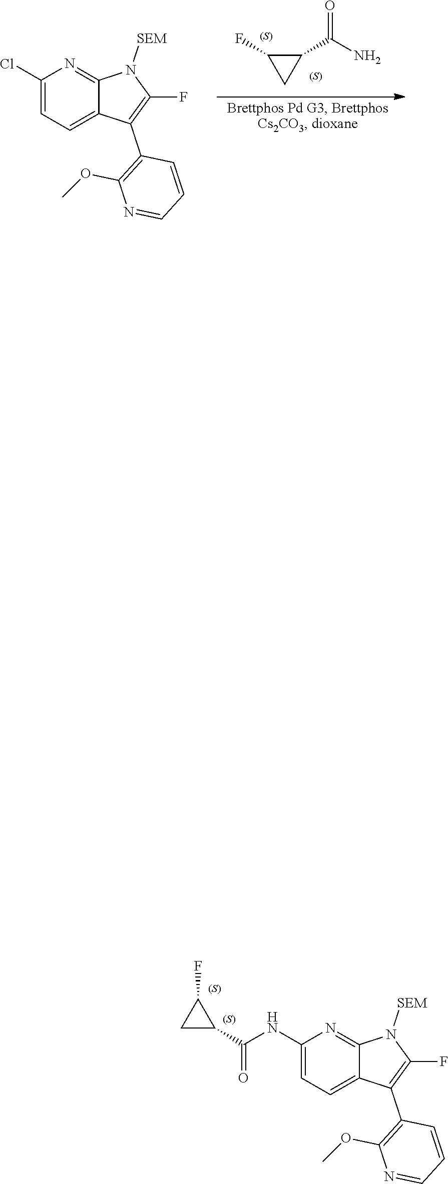

To a solution of 3-(6-chloro-2-fluoro-1-[[2-(trimethylsilyl)ethoxy]methyl]pyrrolo[2,3-b]pyridin-3-yl)-2-methoxypyridine (240.0 mg, 0.59 mmol) in 1,4-dioxane (3.0 mL) was added (1S,2S)-2-fluorocyclopropane-1-carboxamide (182.0 mg, 1.77 mmol), Cs₂CO₃ (575.1 mg, 1.77 mmol), BrettPhos (63.2 mg, 0.12 mmol) and BrettPhos Pd G3 (53.3 mg, 0.06 mmol). The resulting mixture was stirred at 100° C. for 2 h under N₂. After the reaction was completed, the mixture was concentrated under vacuum. The residue was purified by flash column chromatography with petroleum ether/ethyl acetate (60/40, v/v) to afford (1S,2S)-2-fluoro-N-[2-fluoro-3-(2-methoxypyridin-3-yl)-1-[[2-(trimethylsilyl)ethoxy]methyl]pyrrolo[2,3-b]pyridin-6-yl]cyclopropane-1-carboxamide (100.0 mg, 35%) as a yellow solid. LCMS (ESI, m/z): [M+H]⁺=475.2.

### Step 4: Synthesis of (1S,2S)-2-fluoro-N-[2-fluoro-3-(2-methoxypyridin-3-yl)-1H-pyrrolo[2,3-b]pyridin-6-yl]cyclopropane-1-carboxamide (Compound 68)

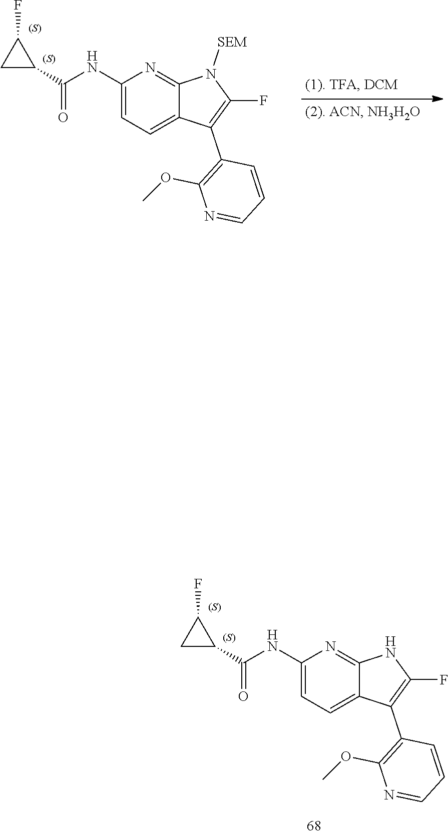

To a solution of (1S,2S)-2-fluoro-N-[2-fluoro-3-(2-methoxypyridin-3-yl)-1-[[2-(trimethylsilyl)ethoxy]methyl]pyrrolo[2,3-b]pyridin-6-yl]cyclopropane-1-carboxamide (80.0 mg, 0.17 mmol) in CH₂Cl₂ (2.0 mL) was added TFA (2.0 mL). The mixture was stirred at room temperature for 2 h. The mixture was concentrated under vacuum. The residue was dissolved in ACN (2.0 mL) and NH₃·H₂O (2.0 mL). The resulting mixture was stirred at room temperature for another 2 h. After the reaction was completed, the resulting mixture was concentrated under vacuum. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 32% B to 42% B in 8 min; 254 nm) to afford (1S,2S)-2-fluoro-N-[2-fluoro-3-(2-methoxypyridin-3-yl)-1H-pyrrolo[2,3-b]pyridin-6-yl]cyclopropane-1-carboxamide (23.8 mg, 41%) as a white solid. LCMS (ESI, m/z): [M+H]⁺=345.1. ¹H NMR (400 MHz, DMSO-d₆): δ 12.55 (s, 1H), 10.64 (s, 1H), 8.18-8.16 (m, 1H), 7.91 (d, J=8.4 Hz, 1H), 7.82-7.77 (m, 2H), 7.11-7.08 (m, 1H), 5.02-4.82 (m, 1H), 3.90 (s, 3H), 2.24-2.20 (m, 1H), 1.69-1.60 (m, 1H), 1.18-1.12 (m, 1H).

### Example S69. Compound 69

### Step 1: Synthesis of 6-chloro-1-((2-(trimethylsilyl)ethoxy)methyl)-1H-pyrrolo[2,3-b]pyridine

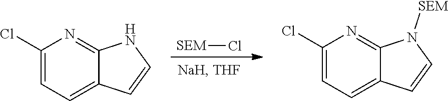

To a solution of 6-chloro-1H-pyrrolo[2,3-b]pyridine (600.0 mg, 3.94 mmol) in THE (5.0 mL) was added NaH (133.6 mg, 60%) at 0° C. under N₂. The resulting mixture was stirred at 0° C. for 1 h. Then SEM-Cl (786.3 mg, 4.72 mmol) was added to the mixture at 0° C. under N₂. The resulting mixture was stirred at 0° C. for another 1 h under N₂. After the reaction was completed, the reaction was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (8/1, v/v) to afford 6-chloro-1-((2-(trimethylsilyl)ethoxy)methyl)-1H-pyrrolo[2,3-b]pyridine (440.0 mg, 39%) as a white solid. LCMS (ESI, m/z): [M+H]⁺=283.1.

### Step 2: Synthesis of (1S,2S)-2-fluoro-N-(1-((2-(trimethylsilyl)ethoxy)methyl)-1H-pyrrolo[2,3-b]pyridin-6-yl)cyclopropane-1-carboxamide

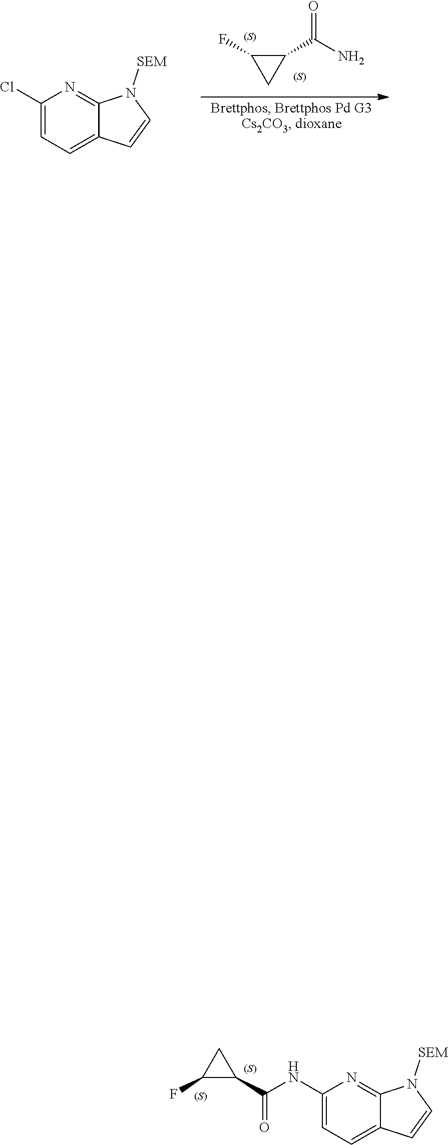

To a solution of 6-chloro-1-((2-(trimethylsilyl)ethoxy)methyl)-1H-pyrrolo[2,3-b]pyridine (440.0 mg, 1.56 mmol) in dioxane (5.0 mL) was added (1S,2S)-2-fluorocyclopropane-1-carboxamide (803.5 mg, 7.80 mmol), Cs₂CO₃ (1.5 g, 4.68 mmol), BrettPhos (167.5 mg, 0.31 mmol) and BrettPhos Pd G3 (141.5 mg, 0.15 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford (1S,2S)-2-fluoro-N-(1-((2-(trimethylsilyl)ethoxy)methyl)-1H-pyrrolo[2,3-b]pyridin-6-yl)cyclopropane-1-carboxamide (170.0 mg, 31%) as a white solid. LCMS (ESI, m/z): [M+H]⁺=350.2.

### Step 3: Synthesis of (1S,2S)—N-(3-bromo-1-((2-(trimethylsilyl)ethoxy)methyl)-1H-pyrrolo[2,3-b]pyridin-6-yl)-2-fluorocyclopropane-1-carboxamide

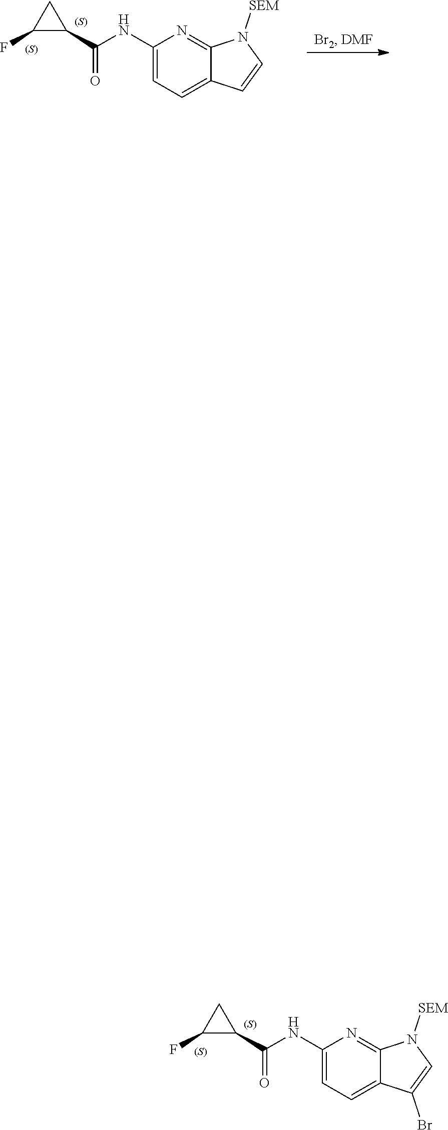

To a solution of (1S,2S)-2-fluoro-N-(1-((2-(trimethylsilyl)ethoxy)methyl)-1H-pyrrolo[2,3-b]pyridin-6-yl)cyclopropane-1-carboxamide (170.0 mg, 0.48 mmol) in DMF (5.0 mL) was added Br₂ (93.0 mg, 0.58 mmol) at 0° C. under N₂. The resulting mixture was stirred at room temperature for 2 h. After the reaction was completed, the mixture was quenched with sat. NaHSO₃ at 0° C. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford (1S,2S)—N-(3-bromo-1-((2-(trimethylsilyl)ethoxy)methyl)-1H-pyrrolo[2,3-b]pyridin-6-yl)-2-fluorocyclopropane-1-carboxamide (70.0 mg, 34%) as a white solid. LCMS (ESI, m/z): [M+H]⁺=428.1.

### Step 4: Synthesis of (1S,2S)—N-[3-(2-cyclopropoxypyridin-3-yl)-1-[[2-(trimethylsilyl)ethoxy]methyl]pyrrolo[2,3-b]pyridin-6-yl]-2-fluorocyclopropane-1-carboxamide

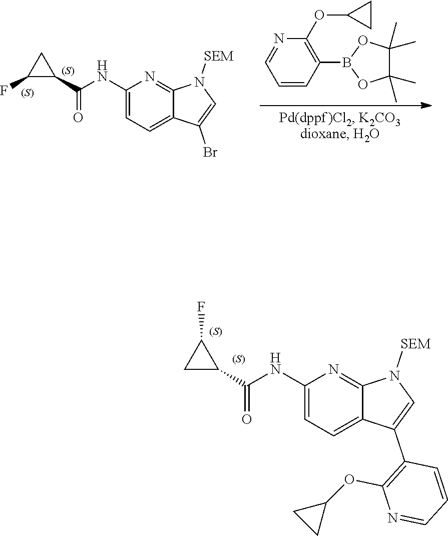

To a solution of (1S,2S)—N-(3-bromo-1-[[2-(trimethylsilyl)ethoxy]methyl]pyrrolo[2,3-b]pyridin-6-yl)-2-fluorocyclopropane-1-carboxamide (70.0 mg, 0.16 mmol) in dioxane/H₂O (5.0/1.0 mL) was added 2-cyclopropoxy-3-(4,4,5,5-tetramethyl-1,3,2-dioxaborolan-2-yl)pyridine (51.3 mg, 0.19 mmol), K₂CO₃ (66.2 mg, 0.48 mmol) and Pd(dppf)Cl₂ (11.7 mg, 0.02 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford (1S,2S)—N-[3-(2-cyclopropoxypyridin-3-yl)-1-[[2-(trimethylsilyl)ethoxy]methyl]pyrrolo[2,3-b]pyridin-6-yl]-2-fluorocyclopropane-1-carboxamide (28.0 mg, 29%) as a white solid. LCMS (ESI, m/z): [M+H]⁺=483.2.

### Step 5: Synthesis of (1S,2S)—N-[3-(2-cyclopropoxypyridin-3-yl)-1H-pyrrolo[2,3-b]pyridin-6-yl]-2-fluorocyclopropane-1-carboxamide (Compound 69)

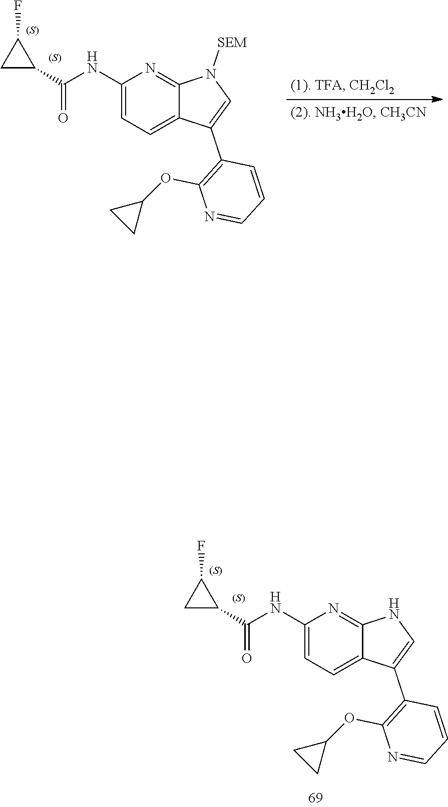

To a solution of (1S,2S)—N-[3-(2-cyclopropoxypyridin-3-yl)-1-[[2-(trimethylsilyl)ethoxy]methyl]pyrrolo[2,3-b]pyridin-6-yl]-2-fluorocyclopropane-1-carboxamide (28.0 mg, 0.06 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in ACN (3.0 mL) was added NH₃·H₂O (3.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 32% B to 47% B in 7 min; 254 nm) to afford (1S,2S)—N-[3-(2-cyclopropoxypyridin-3-yl)-1H-pyrrolo[2,3-b]pyridin-6-yl]-2-fluorocyclopropane-1-carboxamide (7.2 mg, 35%) as a white solid. LCMS (ESI, m/z): [M+H]⁺=353.2. ¹H NMR (400 MHz, DMSO-d₆): δ 11.69 (s, 1H), 10.66 (s, 1H), 8.12-8.05 (m, 2H), 7.99-7.86 (m, 2H), 7.64 (d, J=2.4 Hz, 1H), 7.12-7.09 (m, 1H), 5.03-4.82 (m, 1H), 4.38-4.34 (m, 1H), 2.27-2.20 (m, 1H), 1.70-1.61 (m, 1H), 1.21-1.11 (m, 1H), 0.82-0.74 (m, 2H), 0.71-0.67 (m, 2H).

### Example S70. Compound 70

### Step 1: Synthesis of (1S,2S)—N-[3-[2-(difluoromethoxy)pyridin-3-yl]-1-[[2-(trimethylsilyl)ethoxy]methyl]pyrrolo[2,3-b]pyridin-6-yl]-2-fluorocyclopropane-1-carboxamide

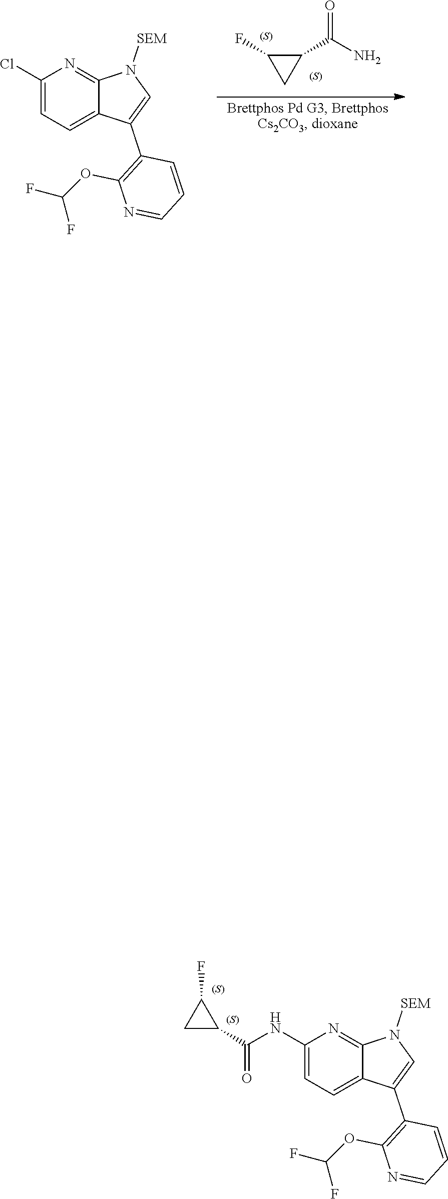

To a solution of 3-(6-chloro-1-[[2-(trimethylsilyl)ethoxy]methyl]pyrrolo[2,3-b]pyridin-3-yl)-2-(difluoromethoxy)pyridine (140.0 mg, 0.33 mmol) in dioxane (3.0 mL) was added (1S,2S)-2-fluorocyclopropane-1-carboxamide (169.4 mg, 1.64 mmol), BrettPhos (35.3 mg, 0.06 mmol), Cs₂CO₃ (321.3 mg, 0.99 mmol) and BrettPhos Pd G3 (29.8 mg, 0.03 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford (1S,2S)—N-[3-[2-(difluoromethoxy)pyridin-3-yl]-1-[[2-(trimethylsilyl)ethoxy]methyl]pyrrolo[2,3-b]pyridin-6-yl]-2-fluorocyclopropane-1-carboxamide (45.0 mg, 28%) as a white solid. LCMS (ESI, m/z): [M+H]⁺=493.2.

### Step 2: Synthesis of (1S,2S)—N-[3-[2-(difluoromethoxy)pyridin-3-yl]-1H-pyrrolo[2,3-b]pyridin-6-yl]-2-fluorocyclopropane-1-carboxamide (Compound 70)

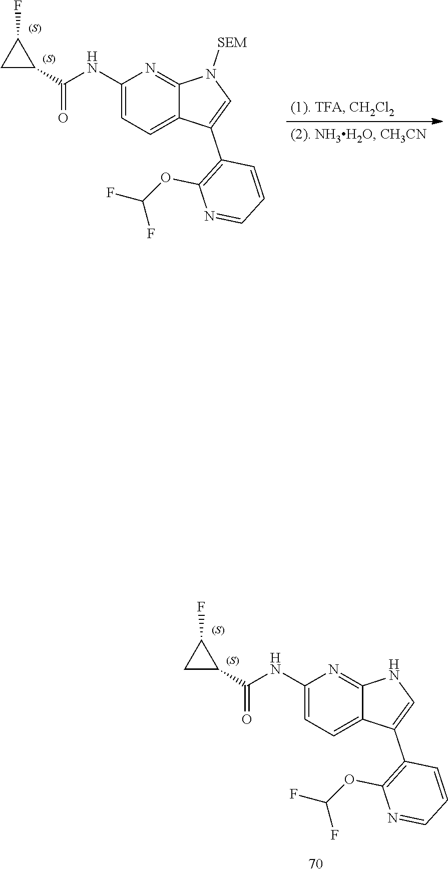

To a solution of (1S,2S)—N-[3-[2-(difluoromethoxy)pyridin-3-yl]-1-[[2-(trimethylsilyl)ethoxy]methyl]pyrrolo[2,3-b]pyridin-6-yl]-2-fluorocyclopropane-1-carboxamide (45.0 mg, 0.09 mmol) in CH₂Cl₂ (1.0 mL) was added TFA (1.0 mL). The resulting mixture was stirred at room temperature for 3 h. The resulting mixture was concentrated under reduced pressure. The residue was dissolved in CH₃CN (1.0 mL) and NH₃·H₂O (1.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 31% B to 61% B in 10 min; 254 nm) to afford (1S,2S)—N-[3-[2-(difluoromethoxy)pyridin-3-yl]-1H-pyrrolo[2,3-b]pyridin-6-yl]-2-fluorocyclopropane-1-carboxamide (6.4 mg, 19%) as a white solid. LCMS (ESI, m/z): [M+H]⁺=363.1. ¹H NMR (400 MHz, DMSO-d₆): δ 11.82 (s, 1H), 10.66 (s, 1H), 8.15-8.09 (m, 3H), 7.97-7.61 (m, 3H), 7.36-7.32 (m, 1H), 5.00-4.81 (m, 1H), 2.24-2.20 (m, 1H), 1.68-1.60 (m, 1H), 1.17-1.14 (m, 1H).

### Example S71. Compound 71

### Step 1: Synthesis of 6-chloro-3-[imidazo[1,2-a]pyridin-6-yl]-1-[[2-(trimethylsilyl)ethoxy]methyl]pyrrolo[2,3-b]pyridine

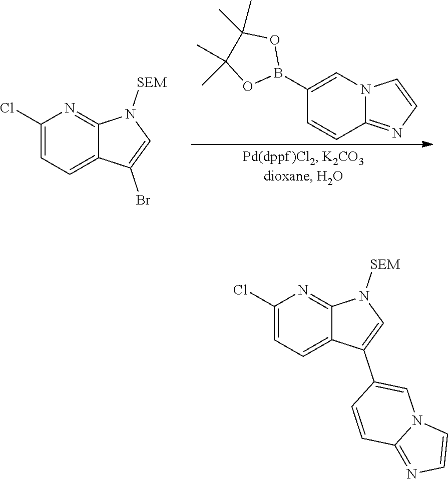 [SMILES: C[Si](C)(C)CCOCn1cc(Br)c2ccc(Cl)nc21 ; CC1(C)OB(c2ccc3nccn3c2)OC1(C)C ; C[Si](C)(C)CCOCn1cc(-c2ccc3nccn3c2)c2ccc(Cl)nc21]

To a solution of 3-bromo-6-chloro-1-[[2-(trimethylsilyl)ethoxy]methyl]pyrrolo[2,3-b]pyridine (300.0 mg, 0.83 mmol) in 1,4-dioxane/H₂O (10.0/2.0 mL) was added 6-(4,4,5,5-tetramethyl-1,3,2-dioxaborolan-2-yl)imidazo[1,2-a]pyridine (202.4 mg, 0.83 mmol), K₂CO₃ (343.9 mg, 2.49 mmol) and Pd(dppf)Cl₂ (60.7 mg, 0.08 mmol) at room temperature under N₂. The resulting mixture was stirred at 80° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 6-chloro-3-[imidazo[1,2-a]pyridin-6-yl]-1-[[2-(trimethylsilyl)ethoxy]methyl]pyrrolo[2,3-b]pyridine (270.0 mg, 81%) as a yellow oil. LCMS (ESI, m/z): [M+H]⁺=399.1.

### Step 2: Synthesis of (1S,2S)-2-fluoro-N-(3-[imidazo[1,2-a]pyridin-6-yl]-1-[[2-(trimethylsilyl)ethoxy]methyl]pyrrolo[2,3-b]pyridin-6-yl)cyclopropane-1-carboxamide

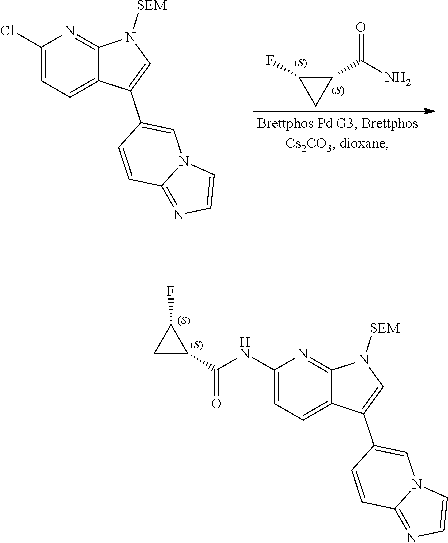

To a solution of 6-chloro-3-[imidazo[1,2-a]pyridin-6-yl]-1-[[2-(trimethylsilyl)ethoxy]methyl]pyrrolo[2,3-b]pyridine (250.0 mg, 0.63 mmol) in 1,4-dioxane (10.0 mL) was added (1S,2S)-2-fluorocyclopropane-1-carboxamide (323.0 mg, 3.13 mmol), BrettPhos (67.3 mg, 0.13 mmol), Cs₂CO₃ (612.5 mg, 1.88 mmol) and BrettPhos Pd G3 (56.8 mg, 0.06 mmol) at room temperature under N₂. The resulting mixture was stirred with microwave at 120° C. for 90 min. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (1S,2S)-2-fluoro-N-(3-[imidazo[1,2-a]pyridin-6-yl]-1-[[2-(trimethylsilyl)ethoxy]methyl]pyrrolo[2,3-b]pyridin-6-yl)cyclopropane-1-carboxamide (160.0 mg, 55%) as a yellow oil. LCMS (ESI, m/z): [M+H]⁺=466.2.

### Step 3: Synthesis of (1S,2S)-2-fluoro-N-(3-[imidazo[1,2-a]pyridin-6-yl]-1H-pyrrolo[2,3-b]pyridin-6-yl)cyclopropane-1-carboxamide (Compound 71)

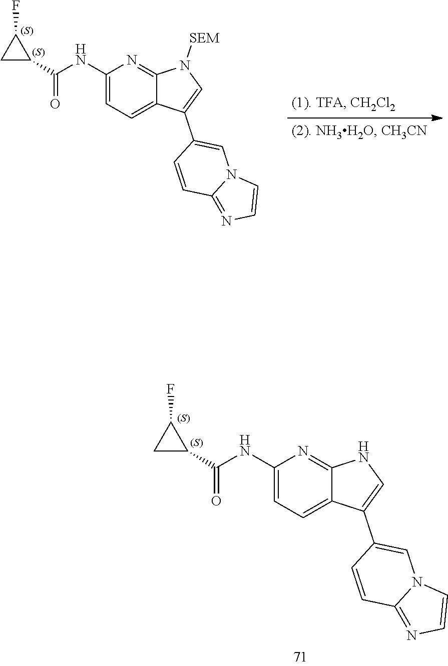

To a solution of (1S,2S)-2-fluoro-N-(3-[imidazo[1,2-a]pyridin-6-yl]-1-[[2-(trimethylsilyl)ethoxy]methyl]pyrrolo[2,3-b]pyridin-6-yl)cyclopropane-1-carboxamide (160.0 mg, 0.34 mmol) in DCM (6.0 mL) was added TFA (6.0 mL) at room temperature. The resulting mixture was stirred at room temperature for 2 h. The resulting mixture was concentrated under reduced pressure. the residue was dissolved in CH₃CN (6.0 mL) and NH₃·H₂O (6.0 mL). The resulting mixture was stirred at room temperature for another 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 17% B to 47% B in 10 min; 254 nm) to afford (1S,2S)-2-fluoro-N-(3-[imidazo[1,2-a]pyridin-6-yl]-1H-pyrrolo[2,3-b]pyridin-6-yl)cyclopropane-1-carboxamide (5.5 mg, 5%) as a white solid. LCMS (ESI, m/z): [M+H]⁺=336.2. ¹H NMR (300 MHz, DMSO-d₆): δ 11.73 (s, 1H), 10.58 (s, 1H), 8.94 (s, 1H), 8.39 (d, J=8.7 Hz, 1H), 7.98 (d, J=6.0 Hz, 2H), 7.86 (s, 1H), 7.67-7.56 (m, 3H), 5.05-4.82 (m, 1H), 2.30-2.21 (m, 1H), 1.71-1.63 (m, 1H), 1.22-1.13 (m, 1H).

### Example S72. Compound 72

### Step 1: Synthesis of 6-bromo-7-methoxyimidazo[1,2-a]pyridine

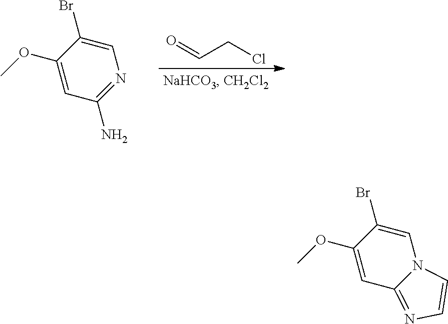

To a solution of 5-bromo-4-methoxypyridin-2-amine (1.0 g, 4.93 mmol) in CH₂Cl₂ (5.0 mL) was added 2-chloroacetaldehyde (2.1 g, 10.84 mmol) and saturated NaHCO₃ solution (10.0 mL) at room temperature. The resulting mixture was stirred at room temperature for 16 h. After the reaction was completed, the reaction was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 6-bromo-7-methoxyimidazo[1,2-a]pyridine (380.0 mg, 33%) as a yellow solid. LCMS (ESI, m/z): [M+H]⁺=227.0.

### Step 2: Synthesis of 6-(6-chloro-1-((2-(trimethylsilyl)ethoxy)methyl)-1H-pyrrolo[2,3-b]pyridin-3-yl)-7-methoxyimidazo[1,2-a]pyridine

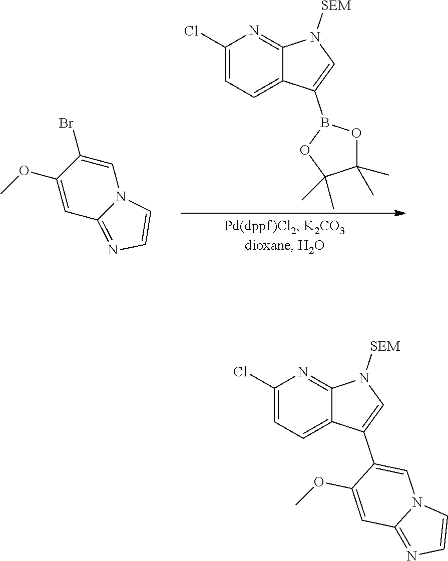 [SMILES: COc1cc2nccn2cc1Br ; CC1(C)OB(c2cn(COCC[Si](C)(C)C)c3nc(Cl)ccc23)OC1(C)C ; COc1cc2nccn2cc1-c1cn(COCC[Si](C)(C)C)c2nc(Cl)ccc12]

To a solution of 6-bromo-7-methoxyimidazo[1,2-a]pyridine (140.0 mg, 0.62 mmol) in dioxane/H₂O (16.0/4.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (252.0 mg, 0.62 mmol), K₂CO₃ (255.6 mg, 1.85 mmol) and Pd(dppf)Cl₂ (45.1 mg, 0.06 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 6-(6-chloro-1-((2-(trimethylsilyl)ethoxy)methyl)-1H-pyrrolo[2,3-b]pyridin-3-yl)-7-methoxyimidazo[1,2-a]pyridine (200.0 mg, 75%) as a brown oil. LCMS (ESI, m/z): [M+H]⁺=429.1.

### Step 3: Synthesis of (1S,2S)-2-fluoro-N-(3-(7-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide

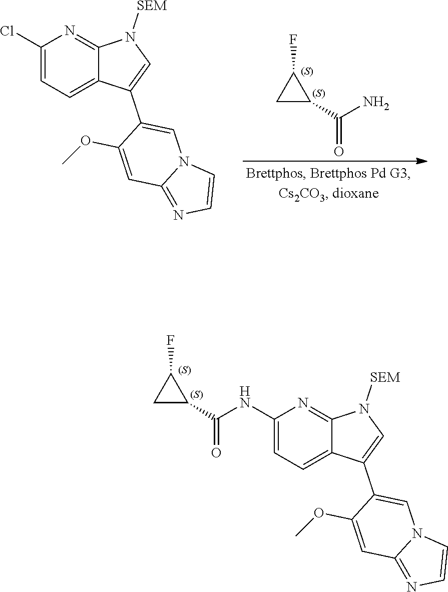

To a solution of 6-(6-chloro-1-((2-(trimethylsilyl)ethoxy)methyl)-1H-pyrrolo[2,3-b]pyridin-3-yl)-7-methoxyimidazo[1,2-a]pyridine (180.0 mg, 0.42 mmol) in dioxane (10.0 mL) was added (1S,2S)-2-fluorocyclopropane-1-carboxamide (216.3 mg, 2.10 mmol), Cs₂CO₃ (401.1 mg, 1.26 mmol), Brettphos (45.0 mg, 0.08 mmol) and BrettPhos Pd G3 (38.0 mg, 0.04 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford (1S,2S)-2-fluoro-N-(3-(7-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide (160.0 mg, 76%) as a yellow solid. LCMS (ESI, m/z): [M+H]⁺=496.2.

### Step 4: Synthesis of (1S,2S)-2-fluoro-N-(3-(7-methoxyimidazo[1,2-a]pyridin-6-yl)-1H-pyrrolo[2,3-b]pyridin-6-yl)cyclopropane-1-carboxamide (Compound 72)

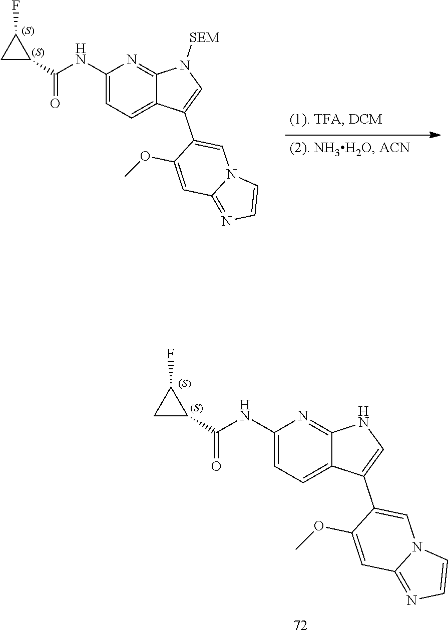

To a solution of (1S,2S)-2-fluoro-N-(3-(7-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide (140.0 mg, 0.28 mmol) in DCM (5.0 mL) was added TFA (5.0 mL) at room temperature. The resulting mixture was stirred at room temperature for 3 h. The resulting mixture was concentrated under reduced pressure. The residue was re-dissolved in ACN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with MeOH/H₂O (56/44, v/v) to afford to afford (1S,2S)-2-fluoro-N-(3-(7-methoxyimidazo[1,2-a]pyridin-6-yl)-1H-pyrrolo[2,3-b]pyridin-6-yl)cyclopropane-1-carboxamide (9.0 mg, 8%) as a white solid. LCMS (ESI, m/z): [M+H]⁺=366.3. ¹H NMR (400 MHz, DMSO-d₆): δ 10.64 (s, 1H), 8.61 (s, 1H), 8.04 (d, J=8.8 Hz, 1H), 7.90 (d, J=8.8 Hz, 1H), 7.78 (s, 1H), 7.60 (s, 1H), 7.40-7.37 (m, 1H), 7.03 (s, 1H), 5.02-4.81 (m, 1H), 3.87 (s, 3H), 2.25-2.19 (m, 1H), 1.70-1.61 (m, 1H), 1.35-1.04 (m, 1H).

### Example S73. Compound 73

### Step 1: Synthesis of 6-bromo-5-chloroimidazo[1,2-a]pyridine

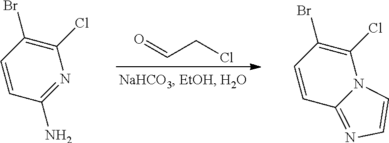

To a solution of 5-bromo-6-chloropyridin-2-amine (6.0 g, 28.92 mmol) in EtOH/H₂O (48.0/12.0 mL) was added NaHCO₃ (2.9 g, 34.71 mmol) and 2-chloroacetaldehyde (6.8 g, 34.71 mmol) at room temperature. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (97/3, v/v) to afford 6-bromo-5-chloroimidazo[1,2-a]pyridine (2.4 g, 35%) as a brown solid. LCMS (ESI, m/z): [M+H]⁺=230.9.

### Step 2: Synthesis of 6-bromo-5-methoxyimidazo[1,2-a]pyridine

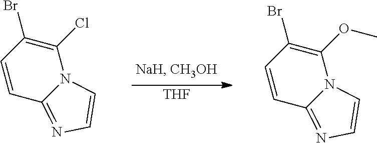

To a solution of 6-bromo-5-chloroimidazo[1,2-a]pyridine (1.0 g, 4.32 mmol) in THE (15.0 mL) was added CH₃OH (207.6 mg, 6.48 mmol) and NaH (345.6 mg, 60%) at room temperature under N₂. The resulting mixture was stirred at 60° C. for 16 h. After the reaction was completed, the resulting mixture was concentrated under reduced pressure. The residue was purified by flash column chromatography with CH₂Cl₂/CH₃OH (96/4, v/v) to afford 6-bromo-5-methoxyimidazo[1,2-a]pyridine (550.0 mg, 56%) as a brown solid. LCMS (ESI, m/z): [M+H]⁺=227.0.

### Step 3: Synthesis of 6-(6-chloro-1-((2-(trimethylsilyl)ethoxy)methyl)-1H-pyrrolo[2,3-b]pyridin-3-yl)-5-methoxyimidazo[1,2-a]pyridine

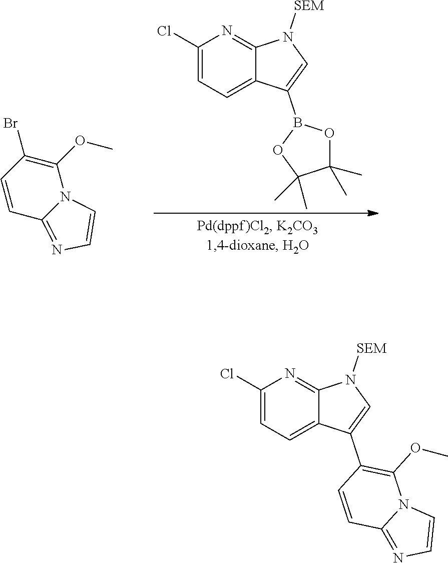 [SMILES: COc1c(Br)ccc2nccn12 ; CC1(C)OB(c2cn(COCC[Si](C)(C)C)c3nc(Cl)ccc23)OC1(C)C ; COc1c(-c2cn(COCC[Si](C)(C)C)c3nc(Cl)ccc23)ccc2nccn12]

To a solution of 6-bromo-5-methoxyimidazo[1,2-a]pyridine (155.0 mg, 0.68 mmol) in 1,4-dioxane/H₂O (5.0/1.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (279.1 mg, 0.68 mmol), K₂CO₃ (283.0 mg, 2.05 mmol) and Pd(dppf)Cl₂ (50.0 mg, 0.07 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 6-(6-chloro-1-((2-(trimethylsilyl)ethoxy)methyl)-1H-pyrrolo[2,3-b]pyridin-3-yl)-5-methoxyimidazo[1,2-a]pyridine (162.0 mg, 52%) as a brown solid. LCMS (ESI, m/z): [M+H]⁺=429.1.

### Step 4: Synthesis of (1S,2S)-2-fluoro-N-(3-(5-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide

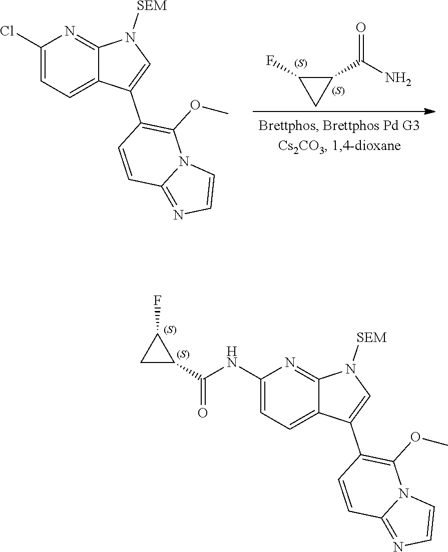

To a solution of 6-(6-chloro-1-((2-(trimethylsilyl)ethoxy)methyl)-1H-pyrrolo[2,3-b]pyridin-3-yl)-5-methoxyimidazo[1,2-a]pyridine (160.0 mg, 0.37 mmol) in 1,4-dioxane (5.0 mL) was added (1S,2S)-2-fluorocyclopropane-1-carboxamide (192.3 mg, 1.87 mmol), Cs₂CO₃ (364.6 mg, 1.12 mmol), BrettPhos (40.0 mg, 0.08 mmol) and BrettPhos Pd G3 (33.8 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford (1S,2S)-2-fluoro-N-(3-(5-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide (140.0 mg, 47%) as a yellow oil. LCMS (ESI, m/z): [M+H]⁺=496.2.

### Step 5: Synthesis of (1S,2S)-2-fluoro-N-(3-(5-methoxyimidazo[1,2-a]pyridin-6-yl)-1H-pyrrolo[2,3-b]pyridin-6-yl)cyclopropane-1-carboxamide (Compound 73)

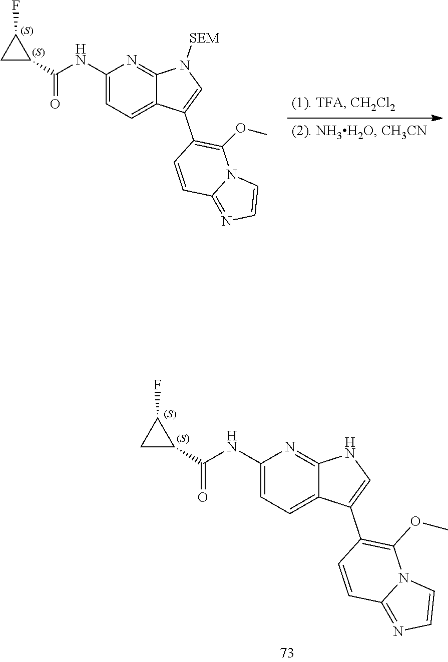

To a solution of (1S,2S)-2-fluoro-N-(3-(5-methoxyimidazo[1,2-a]pyridin-6-yl)-1-((2-(trimethylsilyl)ethoxy)methyl)-1H-pyrrolo[2,3-b]pyridin-6-yl)cyclopropane-1-carboxamide (140.0 mg, 0.28 mmol) in DCM (3.0 mL) was added TFA (3.0 mL) at room temperature. The resulting mixture was stirred at room temperature for 4 h. The resulting mixture was concentrated under reduced pressure. The residue was re-dissolved in ACN (3.0 mL) and NH₃·H₂O (3.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 21% B to 30% B in 8 min; 254 nm) to afford (1S,2S)-2-fluoro-N-(3-(5-methoxyimidazo[1,2-a]pyridin-6-yl)-1H-pyrrolo[2,3-b]pyridin-6-yl) cyclopropane-1-carboxamide (6.3 mg, 6%) as a yellow oil. LCMS (ESI, m/z): [M+H]⁺=366.2. ¹H NMR (300 MHz, DMSO-d₆): δ 11.33 (s, 1H), 10.61 (s, 1H), 8.26 (d, J=8.7 Hz, 1H), 8.02-7.97 (m, 2H), 7.92-7.84 (m, 1H), 7.79 (d, J=2.1 Hz, 1H), 7.62 (d, J=2.1 Hz, 1H), 6.38 (d, J=8.4 Hz, 1H), 5.11-4.78 (m, 1H), 3.75 (s, 3H), 2.23-2.13 (m, 1H), 1.78-1.53 (m, 1H), 1.24-1.08 (m, 1H).

### Example S74. Compound 74

### Step 1: Synthesis of 5-fluoro-2-methoxy-3-(4,4,5,5-tetramethyl-1,3,2-dioxaborolan-2-yl)pyridine

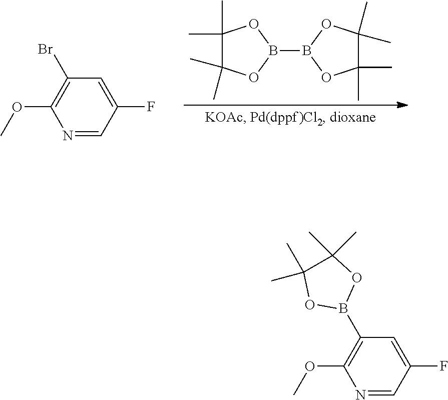

To a solution of 3-bromo-5-fluoro-2-methoxypyridine (1.0 g, 4.85 mmol) in dioxane (30.0 mL) was added bis(pinacolato)diboron (3.7 g, 14.56 mmol), KOAc (1.43 g, 14.56 mmol) and Pd(dppf)Cl₂ (0.4 g, 0.49 mmol) at room temperature under N₂. The resulting mixture was stirred at 80° C. for 16 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by reverse phase flash chromatography with H₂O/ACN (2/1, v/v) to afford 5-fluoro-2-methoxy-3-(4,4,5,5-tetramethyl-1,3,2-dioxaborolan-2-yl)pyridine (600.0 mg, 49%) as a white solid. LCMS (ESI, m/z): [M+H]⁺=254.1.

### Step 2: Synthesis of 3-(6-chloro-1-[[2-(trimethylsilyl)ethoxy]methyl]pyrazolo[3,4-b]pyridin-3-yl)-5-fluoro-2-methoxypyridine

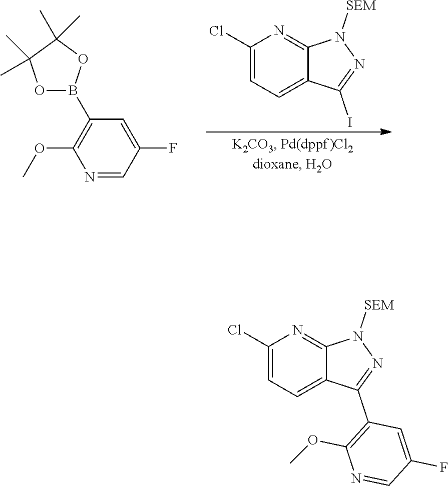 [SMILES: COc1ncc(F)cc1B1OC(C)(C)C(C)(C)O1 ; C[Si](C)(C)CCOCn1nc(I)c2ccc(Cl)nc21 ; COc1ncc(F)cc1-c1nn(COCC[Si](C)(C)C)c2nc(Cl)ccc12]

To a solution of 5-fluoro-2-methoxy-3-(4,4,5,5-tetramethyl-1,3,2-dioxaborolan-2-yl)pyridine (556.0 mg, 2.20 mmol) in dioxane/H₂O (10.0 mL/2.0 mL) was added 6-chloro-3-iodo-1-[[2-(trimethylsilyl)ethoxy]methyl]pyrazolo[3,4-b]pyridine (900.2 mg, 2.20 mmol), K₂CO₃ (910.9 mg, 6.59 mmol) and Pd(dppf)Cl₂ (160.8 mg, 0.22 mmol) at room temperature under N₂. The resulting mixture was stirred at 80° C. for 16 h. After the reaction was completed, the mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 3-(6-chloro-1-[[2-(trimethylsilyl)ethoxy]methyl]pyrazolo[3,4-b]pyridin-3-yl)-5-fluoro-2-methoxypyridine (890.0 mg, 99%) as a white solid. LCMS (ESI, m/z): [M+H]⁺=409.1.

### Step 3: Synthesis of (1S,2S)-2-fluoro-N-[3-(5-fluoro-2-methoxypyridin-3-yl)-1-[[2-(trimethylsilyl)ethoxy]methyl]pyrazolo[3,4-b]pyridin-6-yl]cyclopropane-1-carboxamide

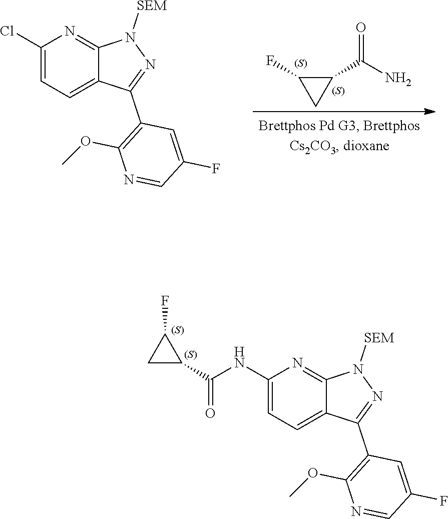

To a solution of 3-(6-chloro-1-[[2-(trimethylsilyl)ethoxy]methyl]pyrazolo[3,4-b]pyridin-3-yl)-5-fluoro-2-methoxypyridine (170.0 mg, 0.42 mmol) in 1,4-dioxane (10.0 mL) was added (1S,2S)-2-fluorocyclopropane-1-carboxamide (214.3 mg, 2.08 mmol), BrettPhos (44.63 mg, 0.08 mmol), Cs₂CO₃ (406.4 mg, 1.25 mmol) and BrettPhos Pd G3 (37.7 mg, 0.04 mmol) at room temperature under N₂. The resulting mixture was stirred at 100° C. for 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(5-fluoro-2-methoxypyridin-3-yl)-1-[[2-(trimethylsilyl)ethoxy]methyl]pyrazolo[3,4-b]pyridin-6-yl]cyclopropane-1-carboxamide (40.0 mg, 20%) as a yellow oil. LCMS (ESI, m/z): [M+H]⁺=476.2.

### Step 4: Synthesis of (1S,2S)-2-fluoro-N-[3-(5-fluoro-2-methoxypyridin-3-yl)-1H-pyrazolo[3,4-b]pyridin-6-yl]cyclopropane-1-carboxamide (Compound 74)

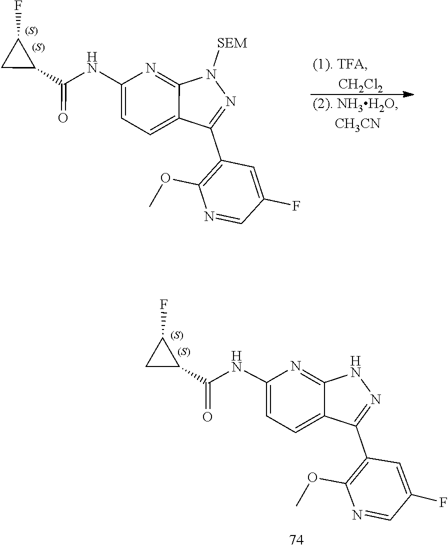

To a solution of (1S,2S)-2-fluoro-N-[3-(5-fluoro-2-methoxypyridin-3-yl)-1-[[2-(trimethylsilyl)ethoxy]methyl]pyrazolo[3,4-b]pyridin-6-yl]cyclopropane-1-carboxamide (120.0 mg, 0.25 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in CH₃CN (5.0 mL) was added NH₃·H₂O (5.0 mL) at room temperature. The resulting mixture was stirred at room temperature for another 2 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (Xselect CSH OBD Column 30×150 mm, 5 um; Mobile Phase A: Water (0.1% FA), Mobile Phase B: ACN; Flow rate: 60 mL/min; Gradient: 27% B to 51% B in 7 min; 254 nm) to afford (1S,2S)-2-fluoro-N-[3-(5-fluoro-2-methoxypyridin-3-yl)-1H-pyrazolo[3,4-b]pyridin-6-yl]cyclopropane-1-carboxamide (19.1 mg, 22%) as a white solid. LCMS (ESI, m/z): [M+H]⁺=346.2. ¹H NMR (400 MHz, DMSO-d₆): δ 13.74 (s, 1H), 10.99 (s, 1H), 8.27-8.24 (m, 2H), 8.04-7.97 (m, 2H), 5.05-4.85 (m, 1H), 3.96 (s, 3H), 2.30-2.23 (m, 1H), 1.72-1.65 (m, 1H), 1.25-1.12 (m, 1H).

### Example S75. Compound 75

### Step 1: Synthesis of 2,4-dimethoxy-3-nitropyridine

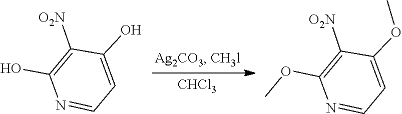

To a solution of 3-nitropyridine-2,4-diol (5.0 g, 32.03 mmol) in CHCl₃ (50.0 mL) was added CH₃I (30.5 g, 256.21 mmol) and Ag₂CO₃ (35.3 g, 128.12 mmol). The resulting mixture was stirred at room temperature for 12 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (63/37, v/v) to afford 2,4-dimethoxy-3-nitropyridine (3.8 g, 64%) as an off-white solid. LCMS (ESI, m/z): [M+H]⁺=185.0.

### Step 2: Synthesis of 2,4-dimethoxypyridin-3-amine

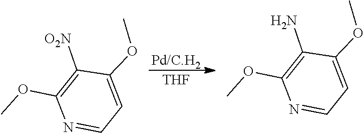

To a solution of 2,4-dimethoxy-3-nitropyridine (3.7 g, 20.92 mmol) in THE (50.0 mL) was added Pd/C (1.1 g, dry) at room temperature. The resulting mixture was stirred at room temperature for 16 h under H₂. After the reaction was completed, the resulting mixture was filtered. The filtrate was concentrated under reduced pressure to afford 2,4-dimethoxypyridin-3-amine (3.0 g, crude) as an off-white solid. LCMS (ESI, m/z): [M+H]⁺=155.1.

### Step 3: Synthesis of 3-bromo-2,4-dimethoxypyridine

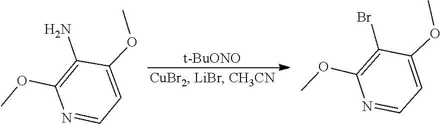

To a solution of 2,4-dimethoxypyridin-3-amine (1.4 g, 9.08 mmol) in CH₃CN (30.0 mL) was added LiBr (2.7 g, 27.23 mmol), t-BuONO (1.7 g, 16.36 mmol) and CuBr₂ (1.6 g, 10.89 mmol). The resulting mixture was stirred at 60° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/ACN (36/64, v/v) to afford 3-bromo-2,4-dimethoxypyridine (1.0 g, 51%) as a red solid. LCMS (ESI, m/z): [M+H]⁺=218.0.

### Step 4: Synthesis of 3-(6-chloro-1-[[2-(trimethylsilyl)ethoxy]methyl]pyrrolo[2,3-b]pyridin-3-yl)-2,4-dimethoxypyridine

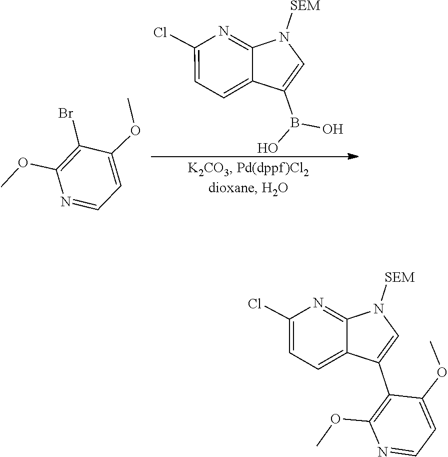 [SMILES: COc1ccnc(OC)c1Br ; C[Si](C)(C)CCOCn1cc(B(O)O)c2ccc(Cl)nc21 ; COc1ccnc(OC)c1-c1cn(COCC[Si](C)(C)C)c2nc(Cl)ccc12]

To a solution of 3-bromo-2,4-dimethoxypyridine (500.0 mg, 2.29 mmol) in H₂O (1.0 mL) and dioxane (10.0 mL) was added K₂CO₃ (950.4 mg, 6.89 mmol), 6-chloro-1-[[2-(trimethylsilyl)ethoxy]methyl]pyrrolo[2,3-b]pyridin-3-ylboronic acid (898.8 mg, 2.75 mmol) and Pd(dppf)Cl₂ (167.7 mg, 0.29 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 3-(6-chloro-1-[[2-(trimethylsilyl)ethoxy]methyl]pyrrolo[2,3-b]pyridin-3-yl)-2,4-dimethoxypyridine (230.0 mg, 24%) as a red oil. LCMS (ESI, m/z): [M+H]⁺=420.1.

### Step 5: Synthesis of (1S,2S)—N-[3-(2,4-dimethoxypyridin-3-yl)-1-[[2-(trimethylsilyl)ethoxy]methyl]pyrrolo[2,3-b]pyridin-6-yl]-2-fluorocyclopropane-1-carboxamide

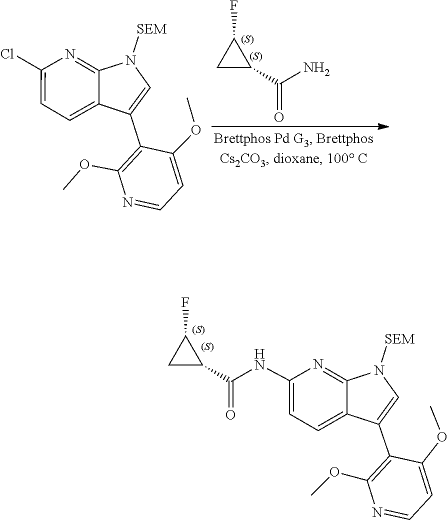

To a solution of 3-(6-chloro-1-[[2-(trimethylsilyl)ethoxy]methyl]pyrrolo[2,3-b]pyridin-3-yl)-2,4-dimethoxypyridine (200.0 mg, 0.47 mmol) in dioxane (10.0 mL) was added (1S,2S)-2-fluorocyclopropane-1-carboxamide (245.8 mg, 2.31 mmol), Brettphos Pd G3 (43.7 mg, 0.08 mmol), BrettPhos (51.2 mg, 0.09 mmol) and Cs₂CO₃ (465.8 mg, 1.49 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-[3-(2,4-dimethoxypyridin-3-yl)-1-[[2-(trimethylsilyl)ethoxy]methyl]pyrrolo[2,3-b]pyridin-6-yl]-2-fluorocyclopropane-1-carboxamide (110.0 mg, 48%) as a yellow solid. LCMS (ESI, m/z): [M+H]⁺=487.2.

### Step 6: Synthesis of (1S,2S)—N-[3-(2,4-dimethoxypyridin-3-yl)-1H-pyrrolo[2,3-b]pyridin-6-yl]-2-fluorocyclopropane-1-carboxamide (Compound 75)

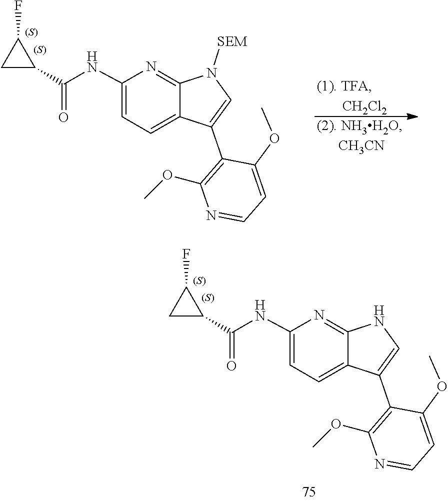

To a solution of (1S,2S)—N-[3-(2,4-dimethoxypyridin-3-yl)-1-[[2-(trimethylsilyl)ethoxy]methyl]pyrrolo[2,3-b]pyridin-6-yl]-2-fluorocyclopropane-1-carboxamide (100.0 mg, 0.19 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 30% B to 39% B in 9 min; 254 nm) to afford (1S,2S)—N-[3-(2,4-dimethoxypyridin-3-yl)-1H-pyrrolo[2,3-b]pyridin-6-yl]-2-fluorocyclopropane-1-carboxamide (28.2 mg, 43%) as a white solid. LCMS (ESI, m/z): [M+H]⁺=357.2. ¹H NMR (300 MHz, DMSO-d₆): δ 11.53 (s, 1H), 10.58 (s, 1H), 8.06 (d, J=6.0 Hz, 1H), 7.83 (d, J=8.4 Hz, 1H), 7.61 (d, J=8.7 Hz, 1H), 7.38 (d, J=2.4 Hz, 1H), 6.88 (d, J=5.7 Hz, 1H), 5.04-4.80 (m, 1H), 3.81 (s, 3H), 3.79 (s, 3H), 2.27-2.18 (m, 1H), 1.72-1.61 (m, 1H), 1.28-1.08 (m, 1H).

### Example S76. Compound 76

### Step 1: Synthesis of 3-bromo-5-fluoro-2,4-dimethoxypyridine

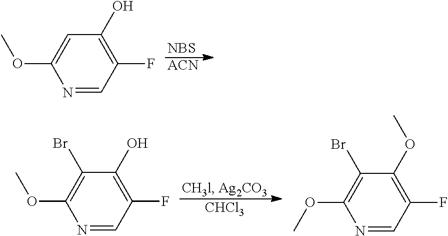

To a solution of 5-fluoro-2-methoxypyridin-4-ol (3.0 g, 20.96 mmol) in ACN (125.0 mL) was added NBS (3.5 g, 19.91 mmol) at room temperature. The resulting mixture was stirred at room temperature for 3 h. After the reaction was completed, the resulting mixture was concentrated under reduced pressure. The residue was dissolved in CHCl₃ (250.0 mL). Then Ag₂CO₃ (15.4 g, 55.85 mmol) and CH₃I (7.9 g, 55.87 mmol) were added to the mixture at room temperature. The resulting mixture was stirred at room temperature for 16 h. After the reaction was completed, the resulting mixture was filtered. The filtrate was concentrated under reduced pressure. The residue was purified by flash column chromatography with petroleum ether/ethyl acetate (10/1, v/v) to afford 3-bromo-5-fluoro-2,4-dimethoxypyridine (3.1 g, 94%) as a white solid. LCMS (ESI, m/z): [M+H]⁺=236.0.

### Step 2: Synthesis of 3-(6-chloro-1-[[2-(trimethylsilyl)ethoxy]methyl]pyrrolo[2,3-b]pyridin-3-yl)-5-fluoro-2,4-dimethoxypyridine

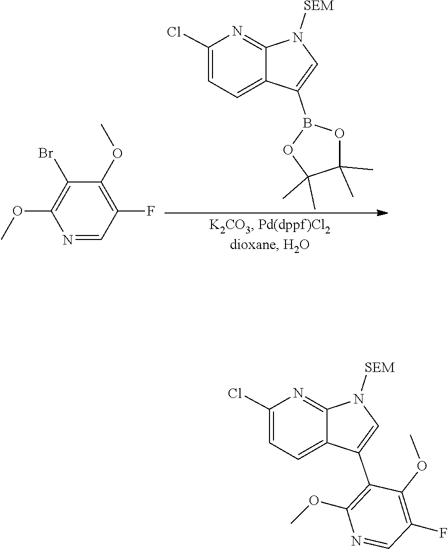 [SMILES: COc1ncc(F)c(OC)c1Br ; CC1(C)OB(c2cn(COCC[Si](C)(C)C)c3nc(Cl)ccc23)OC1(C)C ; COc1ncc(F)c(OC)c1-c1cn(COCC[Si](C)(C)C)c2nc(Cl)ccc12]

To a solution of 3-bromo-5-fluoro-2,4-dimethoxypyridine (173.0 mg, 0.73 mmol) in dioxane/H₂O (10.0 mL/2.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (299.6 mg, 0.73 mmol), K₂CO₃ (303.8 mg, 2.20 mmol) and Pd(dppf)Cl₂ (53.6 mg, 0.07 mmol) at room temperature under N₂. The resulting mixture was stirred at 80° C. for 16 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 3-(6-chloro-1-[[2-(trimethylsilyl)ethoxy]methyl]pyrrolo[2,3-b]pyridin-3-yl)-5-fluoro-2,4-dimethoxypyridine (220.0 mg, 68%) as a colorless oil. LCMS (ESI, m/z): [M+H]⁺=438.1.

### Step 3: Synthesis of (1S,2S)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide

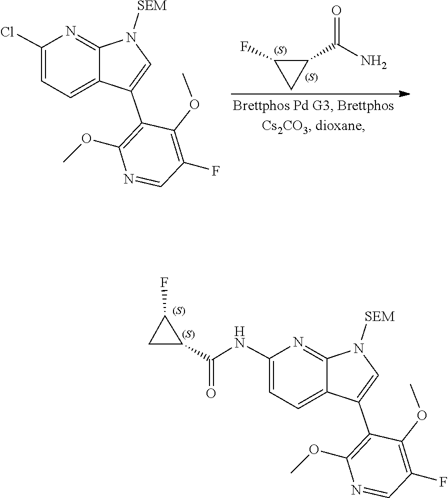

To a solution of 3-(6-chloro-1-[[2-(trimethylsilyl)ethoxy]methyl]pyrrolo[2,3-b]pyridin-3-yl)-5-fluoro-2,4-dimethoxypyridine (200.0 mg, 0.46 mmol) in 1,4-dioxane (15.0 mL) was added (1S,2S)-2-fluorocyclopropane-1-carboxamide (235.4 mg, 2.28 mmol), BrettPhos (49.0 mg, 0.09 mmol), Cs₂CO₃ (446.4 mg, 1.37 mmol) and BrettPhos Pd G3 (41.4 mg, 0.05 mmol) at room temperature under N₂. The resulting mixture was stirred at 100° C. for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide (90.0 mg, 39%) as a yellow oil. LCMS (ESI, m/z): [M+H]⁺=505.2.

### Step 4: Synthesis of (1S,2S)-2-fluoro-N-[3-(5-fluoro-2,4-dimethoxypyridin-3-yl)-1H-pyrrolo[2,3-b]pyridin-6-yl]cyclopropane-1-carboxamide (Compound 76)

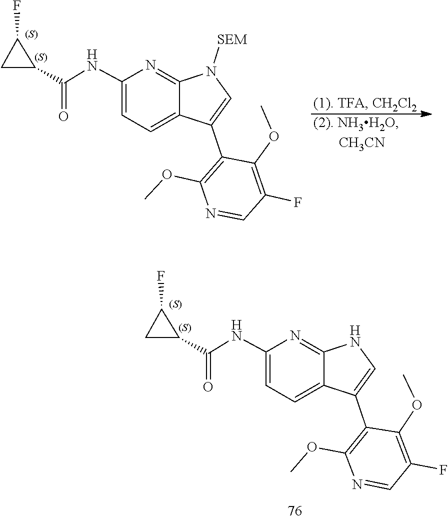

To a solution of (1S,2S)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide (90.0 mg, 0.18 mmol) in CH₂Cl₂ (5.0 mL) was added TFA (5.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the reaction was diluted with H₂ and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (YMC-Actus Triart C18, 30×250 mm, 5 um; Mobile Phase A: Water (10 mmol/L NH₄HCO₃), Mobile Phase B: ACN; Flow rate: 60 mL/min; Gradient: 38% B to 47% B in 8 min; 254 nm) to afford (1S,2S)-2-fluoro-N-[3-(5-fluoro-2,4-dimethoxypyridin-3-yl)-1H-pyrrolo[2,3-b]pyridin-6-yl]cyclopropane-1-carboxamide (19.3 mg, 29%) as a white solid. LCMS (ESI, m/z): [M+H]⁺=375.2. ¹H NMR (400 MHz, DMSO-d₆): δ 11.70 (s, 1H), 10.63 (s, 1H), 8.12 (d, J=3.2 Hz, 1H), 7.87 (d, J=8.4 Hz, 1H), 7.71 (d, J=8.8 Hz, 1H), 7.47 (d, J=2.4 Hz, 1H), 5.02-4.82 (m, 1H), 3.81-3.80 (m, 6H), 2.25-2.22 (m, 1H), 1.70-1.63 (m, 1H), 1.25-1.04 (m, 1H).

### Example S77. Compound 77

### Step 1: Synthesis of 3-(6-chloro-1-[[2-(trimethylsilyl)ethoxy]methyl]pyrazolo[3,4-b]pyridin-3-yl)-2-methoxypyridine

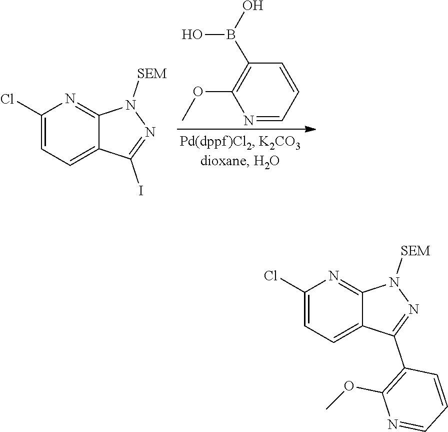

To a solution of 6-chloro-3-iodo-1-[[2-(trimethylsilyl)ethoxy]methyl]pyrazolo[3,4-b]pyridine (300.3 mg, 0.73 mmol) in dioxane/H₂O (10.0 mL/2.0 mL) was added 2-methoxypyridin-3-ylboronic acid (112.0 mg, 0.73 mmol), K₂CO₃ (303.6 mg, 2.20 mmol) and Pd(dppf)Cl₂ (53.6 mg, 0.07 mmol) at room temperature under N₂. The resulting mixture was stirred at 80° C. for 16 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 3-(6-chloro-1-[[2-(trimethylsilyl)ethoxy]methyl]pyrazolo[3,4-b]pyridin-3-yl)-2-methoxypyridine (227.0 mg, 79%) as a white solid. LCMS (ESI, m/z): [M+H]⁺=391.1.

### Step 2: Synthesis of (1S,2S)-2-fluoro-N-[3-(2-methoxypyridin-3-yl)-1-[[2-(trimethylsilyl)ethoxy]methyl]pyrazolo[3,4-b]pyridin-6-yl]cyclopropane-1-carboxamide

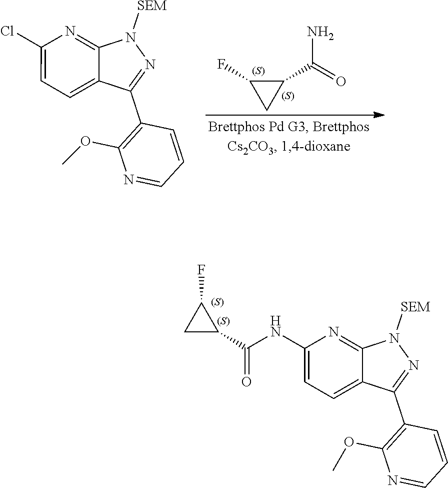

To a solution of 3-(6-chloro-1-[[2-(trimethylsilyl)ethoxy]methyl]pyrazolo[3,4-b]pyridin-3-yl)-2-methoxypyridine (170.0 mg, 0.44 mmol) in 1,4-dioxane (10.0 mL) was added (1S,2S)-2-fluorocyclopropane-1-carboxamide (224.2 mg, 2.17 mmol), BrettPhos (46.7 mg, 0.09 mmol), Cs₂CO₃ (425.1 mg, 1.31 mmol) and BrettPhos Pd G3 (39.4 mg, 0.04 mmol) at room temperature under N₂. The resulting mixture was stirred at 100° C. for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(2-methoxypyridin-3-yl)-1-[[2-(trimethylsilyl)ethoxy]methyl]pyrazolo[3,4-b]pyridin-6-yl]cyclopropane-1-carboxamide (138.0 mg, 70%) as a yellow oil. LCMS (ESI, m/z): [M+H]⁺=458.2.

### Step 3: Synthesis of (1S,2S)-2-fluoro-N-[3-(2-methoxypyridin-3-yl)-1H-pyrazolo[3,4-b]pyridin-6-yl]cyclopropane-1-carboxamide (Compound 77)

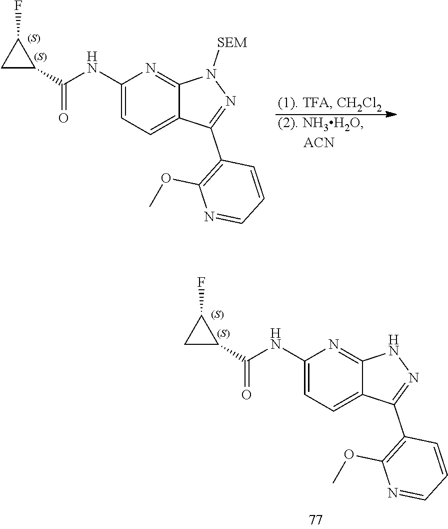

To a solution of (1S,2S)-2-fluoro-N-[3-(2-methoxypyridin-3-yl)-1-[[2-(trimethylsilyl)ethoxy]methyl]pyrazolo[3,4-b]pyridin-6-yl]cyclopropane-1-carboxamide (138.0 mg, 0.30 mmol) in CH₂Cl₂ (5.0 mL) was added TFA (5.0 mL) at room temperature. The resulting mixture was stirred at room temperature for 4 h. The resulting mixture was concentrated under reduced pressure. The residue was dissolved in CH₃CN (5.0 mL) and NH₃·H₂O (5.0 mL). The resulting mixture was stirred at room temperature for another 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 59% B in 10 min; 254 nm) to afford (1S,2S)-2-fluoro-N-[3-(2-methoxypyridin-3-yl)-1H-pyrazolo[3,4-b]pyridin-6-yl]cyclopropane-1-carboxamide (17.3 mg, 18%) as a white solid. LCMS (ESI, m/z): [M+H]⁺=328.2. ¹H NMR (300 MHz, DMSO-d₆): δ 13.60 (s, 1H), 10.96 (s, 1H), 8.29-8.20 (m, 2H), 8.08-8.00 (m, 2H), 7.16-7.12 (m, 1H), 5.07-4.84 (m, 1H), 3.96 (s, 3H), 2.29-2.25 (m, 1H), 1.73-1.60 (m, 1H), 1.23-1.18 (m, 1H).

### Example S78. Compound 78

### Step 1: Synthesis of 6-chloro-3-(2,6-dimethoxyphenyl)-1-((2-(trimethylsilyl)ethoxy)methyl)-1H-pyrazolo[3,4-b]pyridine

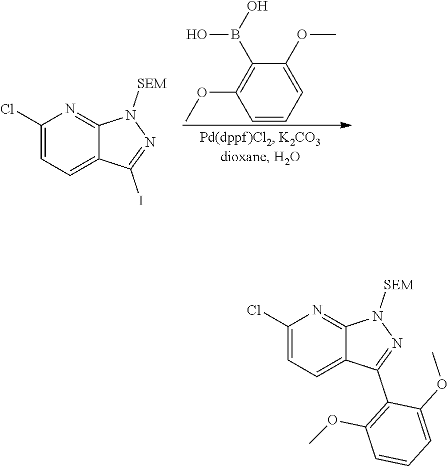

To a solution of 6-chloro-3-iodo-1-((2-(trimethylsilyl)ethoxy)methyl)-1H-pyrazolo[3,4-b]pyridine (562.9 mg, 1.37 mmol) in dioxane/H₂O (16.0/4.0 mL) was added (2,6-dimethoxyphenyl)boronic acid (250.0 mg, 1.37 mmol), K₂CO₃ (569.6 mg, 4.12 mmol) and Pd(dppf)Cl₂ (100.5 mg, 0.14 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-chloro-3-(2,6-dimethoxyphenyl)-1-((2-(trimethylsilyl)ethoxy)methyl)-1H-pyrazolo[3,4-b]pyridine (200.0 mg, 34%) as a brown oil. LCMS (ESI, m/z): [M+H]⁺=420.1.

### Step 2: Synthesis of (1S,2S)—N-(3-(2,6-dimethoxyphenyl)-1-((2-(trimethylsilyl)ethoxy)methyl)-1H-pyrazolo[3,4-b]pyridin-6-yl)-2-fluorocyclopropane-1-carboxamide

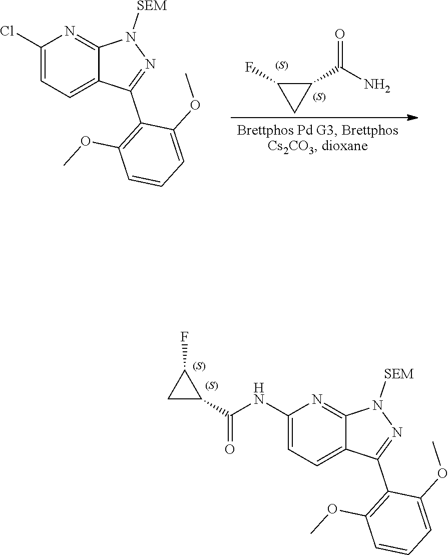

To a solution of 6-chloro-3-(2,6-dimethoxyphenyl)-1-((2-(trimethylsilyl)ethoxy)methyl)-1H-pyrazolo[3,4-b]pyridine (180.0 mg, 0.43 mmol) in dioxane (10.0 mL) was added (1S,2S)-2-fluorocyclopropane-1-carboxamide (220.9 mg, 2.14 mmol), Cs₂CO₃ (418.9 mg, 1.29 mmol), Brettphos (46.0 mg, 0.09 mmol) and BrettPhos Pd G3 (38.9 mg, 0.04 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford (1S,2S)—N-(3-(2,6-dimethoxyphenyl)-1-((2-(trimethylsilyl)ethoxy)methyl)-1H-pyrazolo[3,4-b]pyridin-6-yl)-2-fluorocyclopropane-1-carboxamide (60.0 mg, 19%) as a white solid. LCMS (ESI, m/z): [M+H]⁺=487.2.

### Step 3: Synthesis of (1S,2S)—N-(3-(2,6-dimethoxyphenyl)-1H-pyrazolo[3,4-b]pyridin-6-yl)-2-fluorocyclopropane-1-carboxamide (Compound 78)

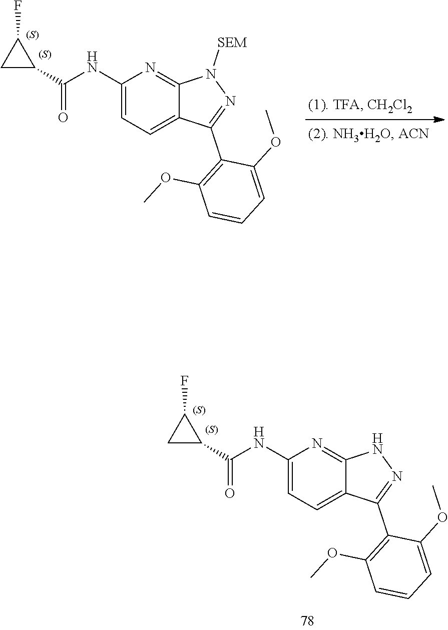

To a solution of (1S,2S)—N-(3-(2,6-dimethoxyphenyl)-1-((2-(trimethylsilyl)ethoxy)methyl)-1H-pyrazolo[3,4-b]pyridin-6-yl)-2-fluorocyclopropane-1-carboxamide (100.0 mg, 0.21 mmol) in CH₂Cl₂ (5.0 mL) was added TFA (5.0 mL) at room temperature. The resulting mixture was stirred at room temperature for 3 h. The resulting mixture was concentrated under reduced pressure. The residue was re-dissolved in ACN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 21% to 51% in 7 min; 254 nm) to afford (1S,2S)—N-(3-(2,6-dimethoxyphenyl)-1H-pyrazolo[3,4-b]pyridin-6-yl)-2-fluorocyclopropane-1-carboxamide (22.1 mg, 30%) as a white solid. LCMS (ESI, m/z): [M+H]⁺=357.2. ¹H NMR (400 MHz, DMSO-d₆): δ 13.31 (s, 1H), 10.92 (s, 1H), 7.92-7.90 (m, 1H), 7.76-7.74 (m, 1H), 7.43-7.39 (m, 1H), 6.80-6.78 (m, 2H), 5.07-4.84 (m, 1H), 3.66 (s, 6H), 2.32-2.21 (m, 1H), 1.75-1.61 (m, 1H), 1.22-1.13 (m, 1H).

### Example S79. Compound 79

### Step 1: Synthesis of 6-chloro-3-(5-fluoro-2-methoxyphenyl)-1-[[2-(trimethylsilyl)ethoxy]methyl]pyrazolo[3,4-b]pyridine

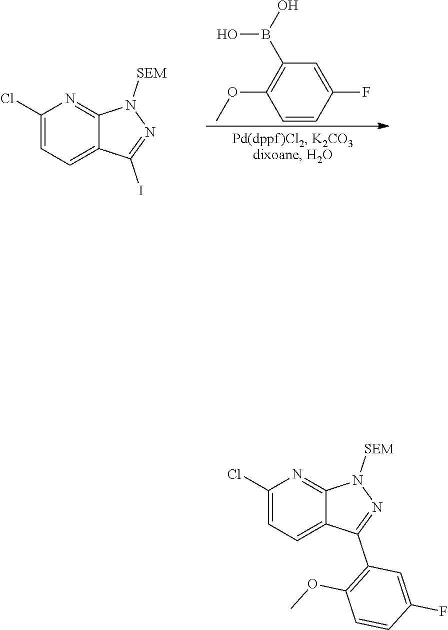

To a solution of 6-chloro-3-iodo-1-[[2-(trimethylsilyl)ethoxy]methyl]pyrazolo[3,4-b]pyridine (1.5 g, 3.66 mmol) in dioxane/H₂O (5.0/1.0 mL) was added 5-fluoro-2-methoxyphenylboronic acid (750.0 mg, 4.39 mmol), K₂CO₃ (1.5 g, 10.9 mmol) and Pd(dppf)Cl₂ (270.0 mg, 0.36 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 6-chloro-3-(5-fluoro-2-methoxyphenyl)-1-[[2-(trimethylsilyl)ethoxy]methyl]pyrazolo[3,4-b]pyridine (1.0 g, 67%) as a colorless oil. LCMS (ESI, m/z): [M+H]⁺=408.1.

### Step 2: Synthesis of N-[3-(5-fluoro-2-methoxyphenyl)-1-[[2-(trimethylsilyl)ethoxy]methyl]pyrazolo[3,4-b]pyridin-6-yl]-1,1-diphenylmethanimine

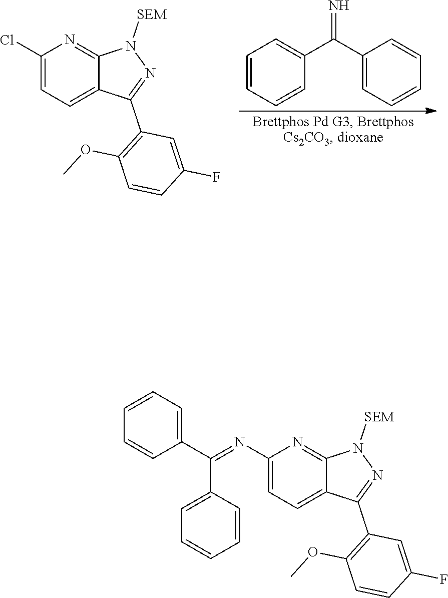 [SMILES: COc1ccc(F)cc1-c1nn(COCC[Si](C)(C)C)c2nc(Cl)ccc12 ; N=C(c1ccccc1)c1ccccc1 ; "Brettphos Pd G3, BrettphosCs2CO3, dioxane" ; COc1ccc(F)cc1-c1nn(COCC[Si](C)(C)C)c2nc(N=C(c3ccccc3)c3ccccc3)ccc12]

To a solution of 6-chloro-3-(5-fluoro-2-methoxyphenyl)-1-[[2-(trimethylsilyl)ethoxy]methyl]pyrazolo[3,4-b]pyridine (980.0 mg, 2.44 mmol) in dioxane (5.0 mL) was added diphenylmethanimine (1.3 g, 7.21 mmol), BrettPhos (257.9 mg, 0.48 mmol), Cs₂CO₃ (2.3 g, 7.21 mmol) and BrettPhos Pd G3 (217.7 mg, 0.24 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (85/15, v/v) to afford N-[3-(5-fluoro-2-methoxyphenyl)-1-[[2-(trimethylsilyl)ethoxy]methyl]pyrazolo[3,4-b]pyridin-6-yl]-1,1-diphenylmethanimine (500.0 mg, 36%) as a yellow solid. LCMS (ESI, m/z): [M+H]⁺=553.2.

### Step 3: Synthesis of 3-(5-fluoro-2-methoxyphenyl)-1-[[2-(trimethylsilyl)ethoxy]methyl]pyrazolo[3,4-b]pyridin-6-amine

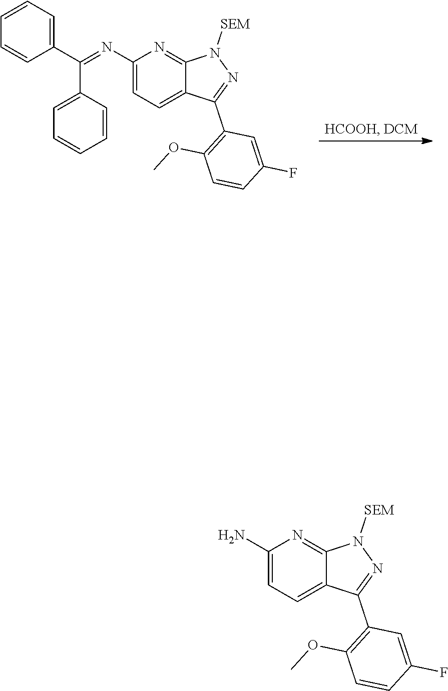

To a solution of N-[3-(5-fluoro-2-methoxyphenyl)-1-[[2-(trimethylsilyl)ethoxy]methyl]pyrazolo[3,4-b]pyridin-6-yl]-1,1-diphenylmethanimine (420.0 mg, 0.76 mmol) in DCM (5.0 mL) was added HCOOH (0.03 mL) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford 3-(5-fluoro-2-methoxyphenyl)-1-[[2-(trimethylsilyl)ethoxy]methyl]pyrazolo[3,4-b]pyridin-6-amine (256.0 mg, 87%) as a white solid. LCMS (ESI, m/z): [M+H]⁺=389.2.

### Step 4: Synthesis of 3-[3-(5-fluoro-2-methoxyphenyl)-1-[[2-(trimethylsilyl)ethoxy]methyl]pyrazolo[3,4-b]pyridin-6-yl]-1-[2-(4-methylpiperazin-1-yl)ethyl]urea

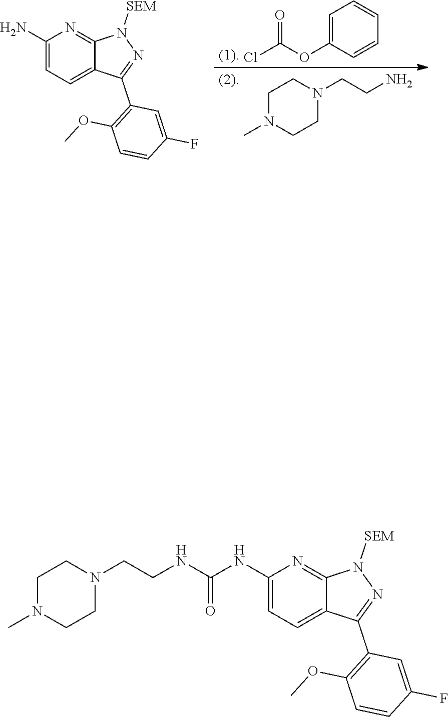

To a solution of 3-(5-fluoro-2-methoxyphenyl)-1-[[2-(trimethylsilyl)ethoxy]methyl]pyrazolo[3,4-b]pyridin-6-amine (140.0 mg, 0.36 mmol) in DCM (5.0 mL) was added pyridine (112.0 mg, 1.41 mmol) and phenyl chloroformate (67.7 mg, 0.43 mmol) at 0° C. under N₂. The resulting mixture was stirred at 0° C. for 1 h. The resulting mixture was concentrated under reduced pressure. Then 2-(4-methylpiperazin-1-yl)ethanamine (206.4 mg, 1.44 mmol) and pyridine (5.0 ml) were added to the residue at 0° C. under N₂. The resulting mixture was stirred at 60° C. for another 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (92/8, v/v) to afford 3-[3-(5-fluoro-2-methoxyphenyl)-1-[[2-(trimethylsilyl)ethoxy]methyl]pyrazolo[3,4-b]pyridin-6-yl]-1-[2-(4-methylpiperazin-1-yl)ethyl]urea (200.0 mg, 99%) as a white solid. LCMS (ESI, m/z): [M+H]⁺=558.3.

### Step 5: Synthesis of 3-[3-(5-fluoro-2-methoxyphenyl)-1H-pyrazolo[3,4-b]pyridin-6-yl]-1-[2-(4-methylpiperazin-1-yl)ethyl]urea (Compound 79)

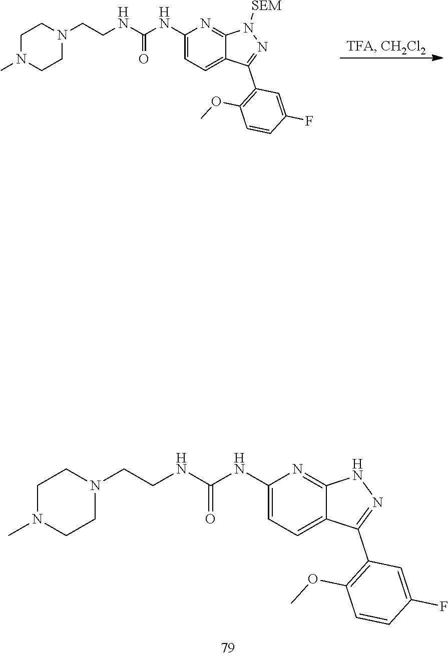

To a solution of 1-(3-(5-fluoro-2-methoxyphenyl)-1-((2-(trimethylsilyl)ethoxy)methyl)-1H-pyrazolo[3,4-b]pyridin-6-yl)-3-(2-(4-methylpiperazin-1-yl)ethyl)urea (130.0 mg, 0.25 mmol) in CH₂Cl₂ (3.0 mL) was added TFA (3.0 mL) at room temperature. The resulting mixture was stirred at room temperature for 3 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 10 min; 254 nm) to afford 3-[3-(5-fluoro-2-methoxyphenyl)-1H-pyrazolo[3,4-b]pyridin-6-yl]-1-[2-(4-methylpiperazin-1-yl)ethyl]urea (9.7 mg, 6%) as a white solid. LCMS (ESI, m/z): [M+H]⁺=428.3. ¹H NMR (400 MHz, DMSO-d₆): δ 13.37 (s, 1H), 9.64 (s, 1H), 8.43 (s, 1H), 8.02 (d, J=8.8 Hz, 1H), 7.42-7.39 (m, 1H), 7.28-7.13 (m, 3H), 3.82 (s, 3H), 3.30 (s, 3H), 3.05-2.83 (m, 4H), 2.79-2.59 (m, 8H).

### Example S80. Compound 80

### Step 1: Synthesis of 3-[2-(dimethylamino)ethyl]-1-[3-(5-fluoro-2-methoxypyridin-3-yl)-1-[[2-(trimethylsilyl)ethoxy]methyl]pyrrolo[2,3-b]pyridin-6-yl]urea

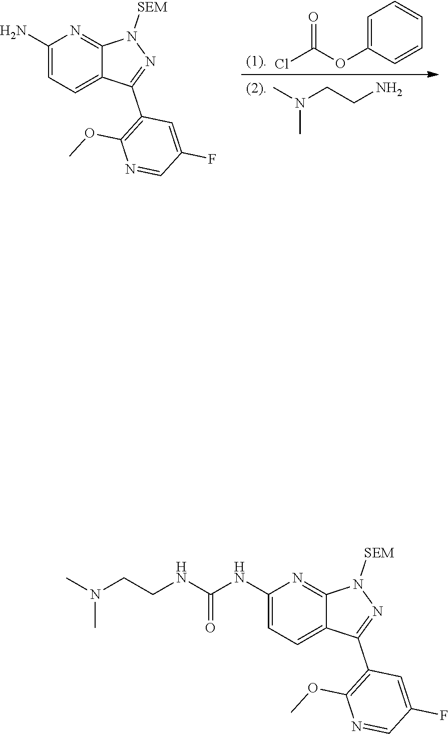

To a solution of 3-(5-fluoro-2-methoxypyridin-3-yl)-1-[[2-(trimethylsilyl)ethoxy]methyl]pyrrolo[2,3-b]pyridin-6-amine (180.0 mg, 0.46 mmol) in DCM (5.0 mL) was added pyridine (144.0 mg, 1.82 mmol) and phenyl chloroformate (89.9 mg, 0.57 mmol) at 0° C. under N₂. The resulting mixture was stirred at 0° C. for 1 h. The resulting mixture was concentrated under reduced pressure. Then (2-aminoethyl)dimethylamine (162.1 mg, 1.84 mmol) and pyridine (5.0 mL) were added to the residue. The resulting mixture was stirred at 60° C. for another 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (92/8, v/v) to afford 3-[2-(dimethylamino)ethyl]-1-[3-(5-fluoro-2-methoxypyridin-3-yl)-1-[[2-(trimethylsilyl)ethoxy]methyl]pyrrolo[2,3-b]pyridin-6-yl]urea (110.0 mg, 47%) as a white solid. LCMS (ESI, m/z): [M+H]⁺=503.3.

### Step 2: Synthesis of 3-[2-(dimethylamino)ethyl]-1-[3-(5-fluoro-2-methoxypyridin-3-yl)-1H-pyrrolo[2,3-b]pyridin-6-yl]urea (Compound 80)

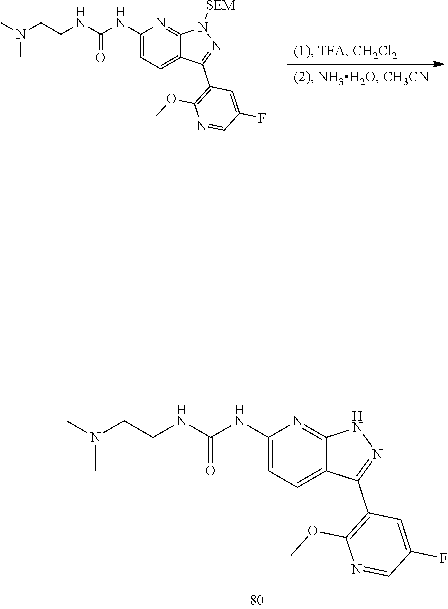 [SMILES: COc1ncc(F)cc1-c1nn(COCC[Si](C)(C)C)c2nc(NC(=O)NCCN(C)C)ccc12 ; "(1), TFA, CH2Cl2" ; "(2), NH3·H2O, CH3CN" ; COc1ncc(F)cc1-c1n[nH]c2nc(NC(=O)NCCN(C)C)ccc12]

To a solution of 3-[2-(dimethylamino)ethyl]-1-[3-(5-fluoro-2-methoxypyridin-3-yl)-1-[[2-(trimethylsilyl)ethoxy]methyl]pyrrolo[2,3-b]pyridin-6-yl]urea (110.0 mg, 0.22 mmol) in CH₂Cl₂ (3.0 mL) was added TFA (3.0 mL) at room temperature. The resulting mixture was stirred at room temperature for 3 h. After the reaction was completed, the resulting mixture was concentrated under reduced pressure. The residue was re-dissolved in CH₃CN (3.0 mL) and NH₃·H₂O (3 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% B to 57% B in 7 min; 254 nm) to afford 3-[2-(dimethylamino)ethyl]-1-[3-(5-fluoro-2-methoxypyridin-3-yl)-1H-pyrrolo[2,3-b]pyridin-6-yl]urea (24.0 mg, 29%) as a white solid. LCMS (ESI, m/z): [M+H]⁺=373.2. ¹H NMR (400 MHz, DMSO-d₆): δ 11.68 (d, J=2.0 Hz, 1H), 9.21 (s, 1H), 8.27 (s, 1H), 8.09 (d, J=8.8 Hz, 1H), 8.03 (d, J=2.8 Hz, 1H), 7.91-7.88 (m, 1H), 7.72 (d, J=2.8 Hz, 1H), 7.18 (d, J=8.8 Hz, 1H), 3.94 (s, 3H), 3.33-3.28 (m, 2H), 2.41-2.38 (m, 2H), 2.20 (s, 6H).

### Example S81. Compound 81

### Step 1: Synthesis of 6-(6-chloro-1-((2-(trimethylsilyl)ethoxy)methyl)-1H-pyrrolo[2,3-b]pyridin-3-yl)-7-methoxy-1-((2-(trimethylsilyl)ethoxy)methyl)-1H-indazole

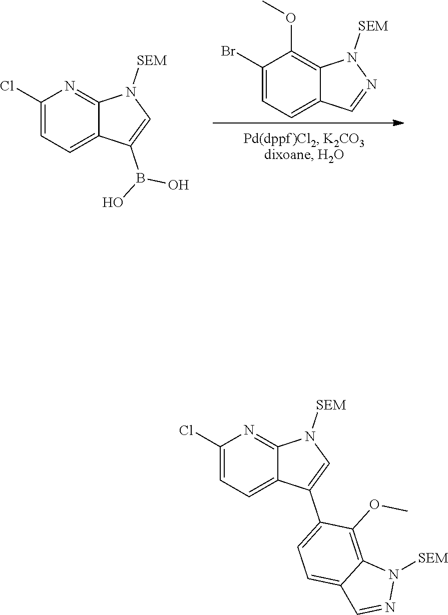

To a solution of (6-chloro-1-((2-(trimethylsilyl)ethoxy)methyl)-1H-pyrrolo[2,3-b]pyridin-3-yl)boronic acid (300.0 mg, 0.92 mmol) in dioxane/H₂O (16.0/4.0 mL) was added 6-bromo-7-methoxy-1-((2-(trimethylsilyl)ethoxy)methyl)-1H-indazole (328.2 mg, 0.92 mmol), K₂CO₃ (380.8 mg, 2.76 mmol) and Pd(dppf)Cl₂ (67.2 mg, 0.09 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 6-(6-chloro-1-((2-(trimethylsilyl)ethoxy)methyl)-1H-pyrrolo[2,3-b]pyridin-3-yl)-7-methoxy-1-((2-(trimethylsilyl)ethoxy)methyl)-1H-indazole (300.0 mg, 58%) as a yellow oil. LCMS (ESI, m/z): [M+H]⁺=559.2.

### Step 2: Synthesis of (1S,2S)-2-fluoro-N-(3-(7-methoxy-1-((2-(trimethylsilyl)ethoxy)methyl)-1H-indazol-6-yl)-1-((2-(trimethylsilyl)ethoxy)methyl)-1H-pyrrolo[2,3-b]pyridin-6-yl)cyclopropane-1-carboxamide

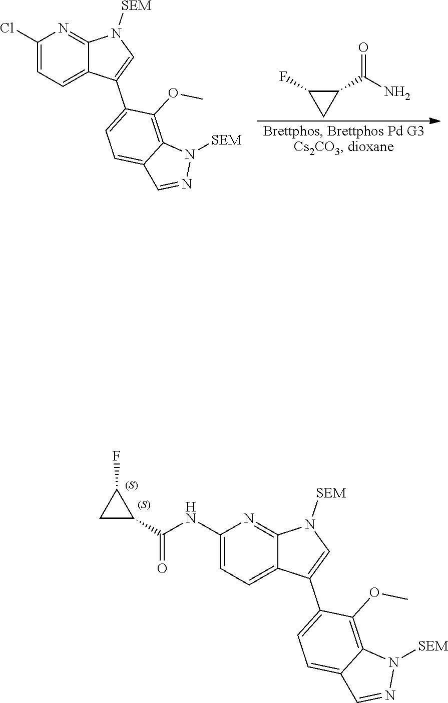

To a solution of 6-(6-chloro-1-((2-(trimethylsilyl)ethoxy)methyl)-1H-pyrrolo[2,3-b]pyridin-3-yl)-7-methoxy-1-((2-(trimethylsilyl)ethoxy)methyl)-1H-indazole (280.0 mg, 0.50 mmol) in dioxane (10.0 mL) was added (1S,2S)-2-fluorocyclopropane-1-carboxamide (258.1 mg, 2.50 mmol), Cs₂CO₃ (489.4 mg, 1.50 mmol), Brettphos (53.8 mg, 0.10 mmol) and BrettPhos Pd G3 (45.4 mg, 0.05 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-(3-(7-methoxy-1-((2-(trimethylsilyl)ethoxy)methyl)-1H-indazol-6-yl)-1-((2-(trimethylsilyl)ethoxy)methyl)-1H-pyrrolo[2,3-b]pyridin-6-yl)cyclopropane-1-carboxamide (110.0 mg, 35%) as a yellow solid. LCMS (ESI, m/z): [M+H]⁺=626.3.

### Step 3: Synthesis of (1S,2S)-2-fluoro-N-(3-(7-methoxy-1H-indazol-6-yl)-1H-pyrrolo[2,3-b]pyridin-6-yl)cyclopropane-1-carboxamide (Compound 81)

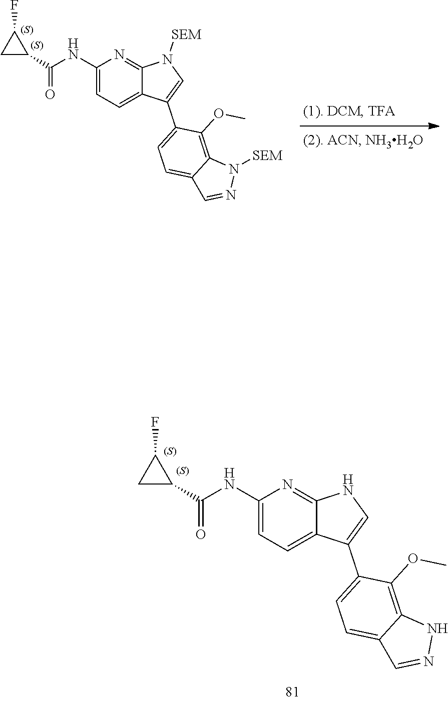

To a solution of (1S,2S)-2-fluoro-N-(3-(7-methoxy-1-((2-(trimethylsilyl)ethoxy)methyl)-1H-indazol-6-yl)-1-((2-(trimethylsilyl)ethoxy)methyl)-1H-pyrrolo[2,3-b]pyridin-6-yl)cyclopropane-1-carboxamide (100.0 mg, 0.16 mmol) in DCM (5.0 mL) was added TFA (5.0 mL) at room temperature. The resulting mixture was stirred at room temperature for 3 h. The resulting mixture was concentrated under reduced pressure. The residue was dissolved in ACN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 21% to 43% in 7 min; 254 nm) to afford (1S,2S)-2-fluoro-N-(3-(7-methoxy-1H-indazol-6-yl)-1H-pyrrolo[2,3-b]pyridin-6-yl)cyclopropane-1-carboxamide (10.8 mg, 18%) as a white solid. LCMS (ESI, m/z): [M+H]⁺=366.2. ¹H NMR (300 MHz, DMSO-d₆): δ 13.32-13.29 (m, 1H), 11.71 (s, 1H), 10.67 (s, 1H), 8.10-8.06 (m, 2H), 7.93 (d, J=8.4 Hz, 1H), 7.67 (s, 1H), 7.55 (d, J=8.1 Hz, 1H), 7.32 (d, J=8.4 Hz, 1H), 5.07-4.81 (m, 1H), 3.68 (s, 3H), 2.28-2.21 (m, 1H), 1.71-1.62 (m, 1H), 1.24-1.19 (m, 1H).

### Example S82. Compound 82

### Step 1: Synthesis of 5-(6-chloro-1-[[2-(trimethylsilyl)ethoxy]methyl]pyrazolo[3,4-b]pyridin-3-yl)-6-methoxy-1-[[2-(trimethylsilyl)ethoxy]methyl]indazole

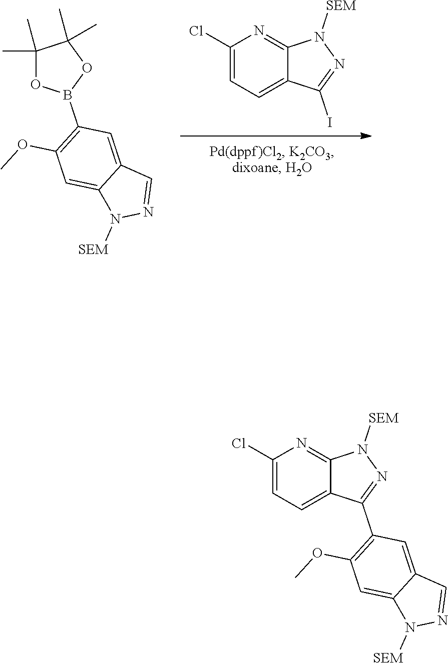 [SMILES: COc1cc2c(cnn2COCC[Si](C)(C)C)cc1B1OC(C)(C)C(C)(C)O1 ; C[Si](C)(C)CCOCn1nc(I)c2ccc(Cl)nc21 ; "Pd(dppf)Cl2, K2CO3,dixoane, H2O" ; COc1cc2c(cnn2COCC[Si](C)(C)C)cc1-c1nn(COCC[Si](C)(C)C)c2nc(Cl)ccc12]

To a mixture of 6-methoxy-5-(4,4,5,5-tetramethyl-1,3,2-dioxaborolan-2-yl)-1-[[2-(trimethylsilyl) ethoxy]methyl]indazole (800.0 mg, 1.97 mmol) and 6-chloro-3-iodo-1-[[2-(trimethylsilyl)ethoxy]methyl] pyrazolo[3,4-b]pyridine (810.5 mg, 1.97 mmol) in dioxane/H₂O (10.0 mL/2.0 mL) was added K₂CO₃ (820.2 mg, 5.93 mmol) and Pd(dppf)Cl₂ (144.7 mg, 0.19 mmol) at room temperature under N₂. The resulting mixture was stirred at 80° C. for 16 h under N₂. After the reaction was completed, the reaction was diluted with water at room temperature, extracted with ethyl acetate. The combined organic layer was washed with brine, dried over anhydrous sodium sulfate and filtered. The resulting mixture was concentrated under reduced pressure. The residue was purified by flash column chromatography with petroleum ether/EtOAc (3/1, v/v) to afford 5-(6-chloro-1-[[2-(trimethylsilyl)ethoxy]methyl]pyrazolo[3,4-b]pyridin-3-yl)-6-methoxy-1-[[2-(trimethylsilyl) ethoxy]methyl]indazole (520.0 mg, 47%) as a yellow oil. LCMS (ESI, m/z): [M+H]⁺=560.2

### Step 2: Synthesis of (1S,2S)-2-fluoro-N-[3-(6-methoxy-1-[[2-(trimethylsilyl)ethoxy]methyl]indazol-5-yl)-1-[[2-(trimethylsilyl)ethoxy]methyl]pyrazolo[3,4-b]pyridin-6-yl]cyclopropane-1-carboxamide

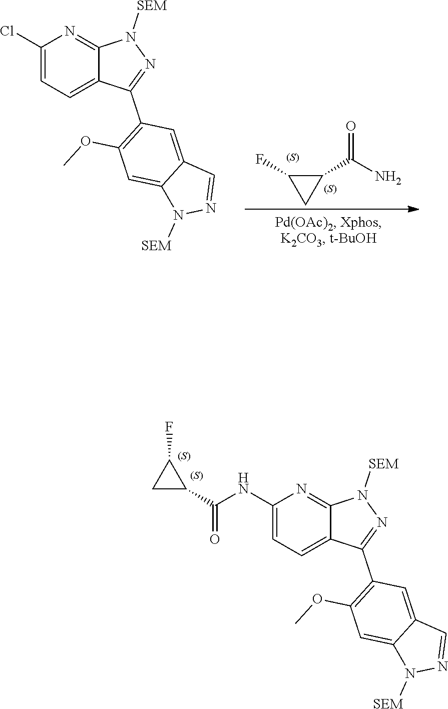

To a mixture of 5-(6-chloro-1-[[2-(trimethylsilyl)ethoxy]methyl]pyrazolo[3,4-b]pyridin-3-yl)-6-methoxy-1-[[2-(trimethylsilyl)ethoxy]methyl]indazole (260.0 mg, 0.46 mmol) and (1S,2S)-2-fluorocyclopropane-1-carboxamide (239.2 mg, 2.32 mmol) in t-BuOH (5.0 mL) was added Pd(OAc)₂ (10.4 mg, 0.04 mmol), K₂CO₃ (192.4 mg, 1.39 mmol) and Xphos (25.5 mg, 0.05 mmol) at room temperature under N₂. The resulting mixture was stirred at 100° C. for 16 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (3/1, v/v) to afford (1S,2S)-2-fluoro-N-[3-(6-methoxy-1-[[2-(trimethylsilyl)ethoxy]methyl] indazol-5-yl)-1-[[2-(trimethylsilyl)ethoxy]methyl]pyrazolo[3,4-b]pyridin-6-yl]cyclopropane-1-carboxamide (220.0 mg, 75%) as a yellow solid. LCMS (ESI, m/z): [M+H]⁺=627.3.

### Step 3: Synthesis of (1S,2S)-2-fluoro-N-[3-(6-methoxy-1H-indazol-5-yl)-1H-pyrazolo[3,4-b]pyridin-6-yl]cyclopropane-1-carboxamide (Compound 82)

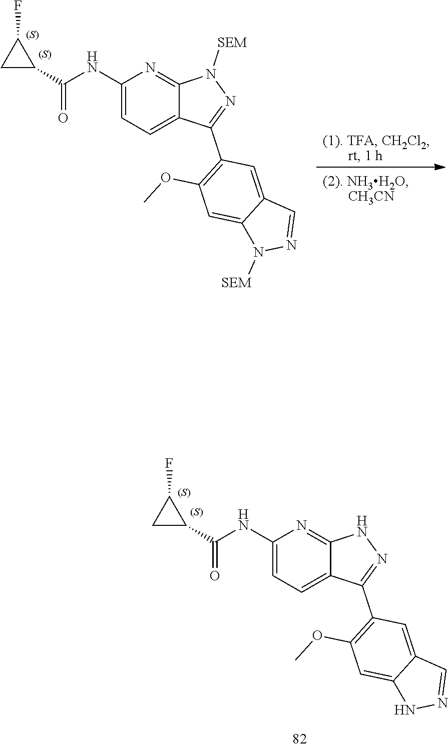

To a solution of (1S,2S)-2-fluoro-N-[3-(6-methoxy-1-[[2-(trimethylsilyl)ethoxy]methyl]indazol-5-yl)-1-[[2-(trimethylsilyl)ethoxy]methyl]pyrazolo[3,4-b]pyridin-6-yl]cyclopropane-1-carboxamide (200.0 mg, 0.31 mmol) in DCM (3.0 mL) was added TFA (3.0 mL). The resulting mixture was stirred at room temperature for 2 h. The resulting mixture was concentrated under reduced pressure. To the above residue was added ACN (1.0 mL) and NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 33% B in 9 min; 254/220 nm) to afford (1S,2S)-2-fluoro-N-[3-(6-methoxy-1H-indazol-5-yl)-1H-pyrazolo[3,4-b]pyridin-6-yl]cyclopropane-1-carboxamide (12.2 mg, 10%) as a white solid. LCMS (ESI, m/z): [M+H]⁺=367.1. ¹H NMR (300 MHz, DMSO-d₆): δ 13.38 (s, 1H), 12.94 (s, 1H), 10.95 (s, 1H), 8.08-7.91 (m, 4H), 7.11 (s, 1H), 5.07-4.83 (m, 1H), 3.87 (s, 3H), 2.27-2.20 (m, 1H), 1.74-1.63 (m, 1H), 1.24-1.18 (m, 1H).

### Example S83. Compound 83

### Step 1: Synthesis of 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine

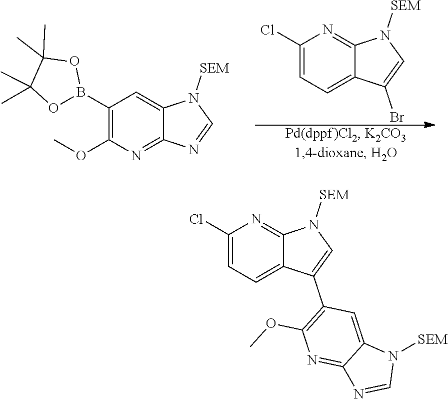 [SMILES: COc1nc2ncn(COCC[Si](C)(C)C)c2cc1B1OC(C)(C)C(C)(C)O1 ; C[Si](C)(C)CCOCn1cc(Br)c2ccc(Cl)nc21 ; COc1nc2ncn(COCC[Si](C)(C)C)c2cc1-c1cn(COCC[Si](C)(C)C)c2nc(Cl)ccc12]

To a solution of 5-methoxy-6-(4,4,5,5-tetramethyl-1,3,2-dioxaborolan-2-yl)-1-[[2-(trimethylsilyl)ethoxy]methyl]imidazo[4,5-b]pyridine (500.0 mg, 1.23 mmol) in 1,4-dioxane/H₂O (5.0/1.0 mL) was added 3-bromo-6-chloro-1-[[2-(trimethylsilyl)ethoxy]methyl]pyrrolo[2,3-b]pyridine (446.1 mg, 1.23 mmol), K₂CO₃ (511.4 mg, 3.70 mmol) and Pd(dppf)Cl₂ (180.5 mg, 0.25 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine (300.0 mg, 43%) as a yellow solid. LCMS (ESI, m/z): [M+H]⁺=560.2.

### Step 2: Synthesis of (1S,2S)-2-fluoro-N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropane-1-carboxamide

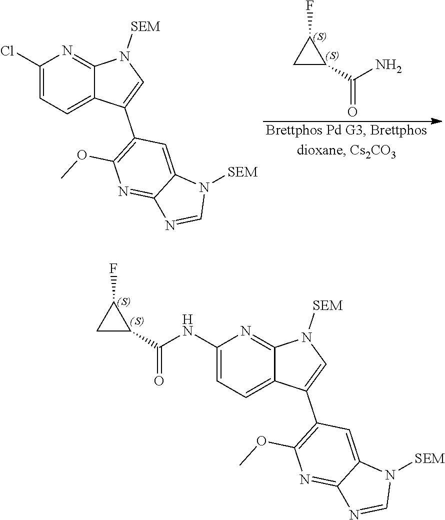

To a solution of 6-chloro-3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridine (300.0 mg, 0.50 mmol) in dioxane (4.0 mL) was added (1S,2S)-2-fluorocyclopropane-1-carboxamide (154.8 mg, 1.49 mmol), BrettPhos (53.6 mg, 0.10 mmol), Cs₂CO₃ (488.5 mg, 1.49 mmol) and BrettPhos Pd G3 (45.3 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (39/61, v/v) to afford (1S,2S)-2-fluoro-N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropane-1-carboxamide (300.0 mg, 76%) as a yellow solid. LCMS (ESI, m/z): [M+H]⁺=627.3.

### Step 3: Synthesis of (1S,2S)-2-fluoro-N-(3-[5-methoxy-3H-imidazo[4,5-b]pyridin-6-yl]-1H-pyrrolo[2,3-b]pyridin-6-yl)cyclopropane-1-carboxamide (Compound 83)

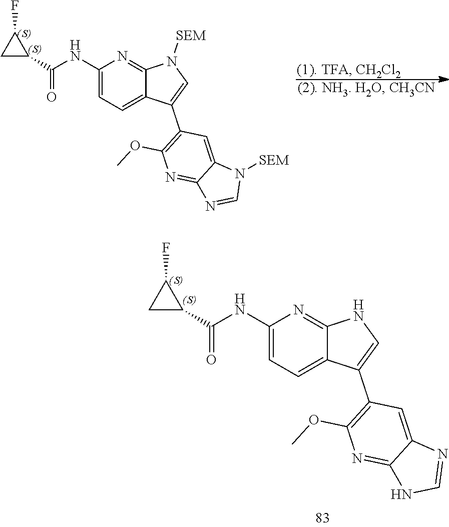

To a solution of (1S,2S)-2-fluoro-N-[3-(5-methoxy-1-[[2-(trimethylsilyl)ethoxy]methyl]imidazo[4,5-b]pyridin-6-yl)-1-[[2-(trimethylsilyl)ethoxy]methyl]pyrrolo[2,3-b]pyridin-6-yl]cyclopropane-1-carboxamide (280.0 mg, 0.44 mmol) in CH₂Cl₂ (2.0 mL) was added TFA (2.0 mL) at room temperature. The resulting mixture was stirred at room temperature for 3 h. The resulting mixture was concentrated under reduced pressure. The residue was re-dissolved in CH₃CN (2.0 mL) and NH₃·H₂O (2.0 mL). The resulting mixture was stirred at room temperature for another 16 h. After the reaction was completed, the resulting mixture was concentrated under reduced pressure, then the was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 21% B to 29% B in 10 min; 254 nm) to afford (1S,2S)-2-fluoro-N-(3-[5-methoxy-3H-imidazo[4,5-b]pyridin-6-yl]-1H-pyrrolo[2,3-b]pyridin-6-yl)cyclopropane-1-carboxamide (8.1 mg, 5%) as a white solid. LCMS (ESI, m/z): [M+H]⁺=367.1. ¹H NMR (400 MHz, DMSO-d₆): δ 13.18-12.18 (m, 1H), 11.60 (s, 1H), 10.61 (s, 1H), 8.35-7.75 (m, 4H), 7.70-7.55 (m, 1H), 5.05-4.70 (m, 1H), 3.95 (s, 3H), 2.29-2.15 (m, 1H), 1.71-1.50 (m, 1H), 1.21-1.08 (m, 1H).

### Example S84. Compound 84

### Step 1: Synthesis of 5-bromo-2-(tert-butylsulfanyl)-4-methoxybenzaldehyde

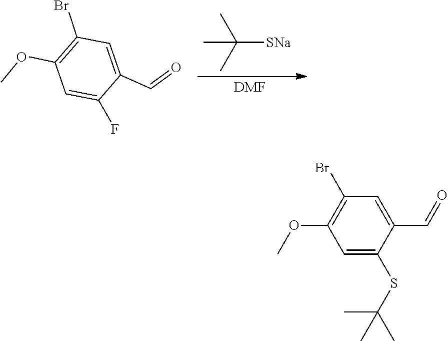

To a solution of 5-bromo-2-fluoro-4-methoxybenzaldehyde (500.0 mg, 2.14 mmol) in DMF (10.0 mL) was added (tert-butylsulfanyl)sodium (361.0 mg, 3.21 mmol) at 0° C. under N₂. The resulting mixture was stirred at 0° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 5-bromo-2-(tert-butylsulfanyl)-4-methoxybenzaldehyde (600.0 mg, crude) as a yellow solid. LCMS (ESI, m/z): [M+H]⁺=303.0.

### Step 2: Synthesis of (E)-N-[[5-bromo-2-(tert-butylsulfanyl)-4-methoxyphenyl]methylidene]hydroxylamine

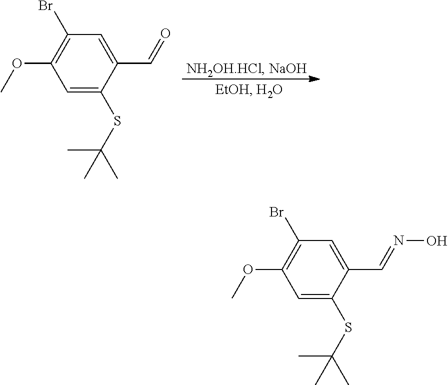 [SMILES: COc1cc(SC(C)(C)C)c(C=O)cc1Br ; COc1cc(SC(C)(C)C)c(/C=N/O)cc1Br]

To a solution of 5-bromo-2-(tert-butylsulfanyl)-4-methoxybenzaldehyde (600.0 mg, 1.98 mmol) in EtOH/H₂O (3.0/9.0 mL) was added NH₂OH HCl (206.2 mg, 2.96 mmol) and NaOH (237.4 mg, 5.93 mmol) at 0° C. under N₂. The resulting mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford (E)-N-[[5-bromo-2-(tert-butylsulfanyl)-4-methoxyphenyl]methylidene]hydroxylamine (510.0 mg, crude) as a yellow solid. LCMS (ESI, m/z): [M+H]⁺=318.0.

### Step 3: Synthesis of 5-bromo-6-methoxy-1,2-benzothiazole

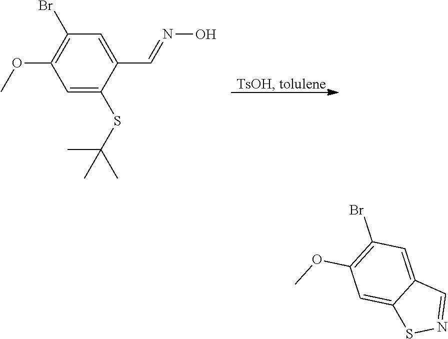 [SMILES: COc1cc(SC(C)(C)C)c(/C=N/O)cc1Br ; COc1cc2sncc2cc1Br]

To a solution of (E)-N-[[5-bromo-2-(tert-butylsulfanyl)-4-methoxyphenyl]methylidene]hydroxylamine (500.0 mg, 1.57 mmol) in toluene (5.0 mL) was added TsOH (54.1 mg, 0.31 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford 5-bromo-6-methoxy-1,2-benzothiazole (150.0 mg, 39%) as a yellow solid. LCMS (ESI, m/z): [M+H]⁺=243.9.

### Step 4: Synthesis of 5-(6-chloro-1-[[2-(trimethylsilyl)ethoxy]methyl]pyrrolo[2,3-b]pyridin-3-yl)-6-methoxy-1,2-benzothiazole

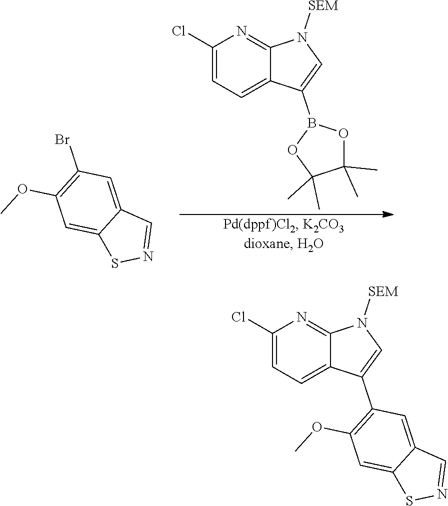 [SMILES: COc1cc2sncc2cc1Br ; CC1(C)OB(c2cn(COCC[Si](C)(C)C)c3nc(Cl)ccc23)OC1(C)C ; COc1cc2sncc2cc1-c1cn(COCC[Si](C)(C)C)c2nc(Cl)ccc12]

To a solution of 5-bromo-6-methoxy-1,2-benzothiazole (130.0 mg, 0.53 mmol) in dioxane/H₂O (5.0/1.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (261.2 mg, 0.64 mmol), K₂CO₃ (220.8 mg, 1.60 mmol) and Pd(dppf)Cl₂ (40.0 mg, 0.05 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 5-(6-chloro-1-[[2-(trimethylsilyl)ethoxy]methyl]pyrrolo[2,3-b]pyridin-3-yl)-6-methoxy-1,2-benzothiazole (220.0 mg, 92%) as a yellow solid. LCMS (ESI, m/z): [M+H]⁺=446.1.

### Step 5: Synthesis of (1S,2S)-2-fluoro-N-[3-(6-methoxy-1,2-benzothiazol-5-yl)-1-[[2-(trimethylsilyl)ethoxy]methyl]pyrrolo[2,3-b]pyridin-6-yl]cyclopropane-1-carboxamide

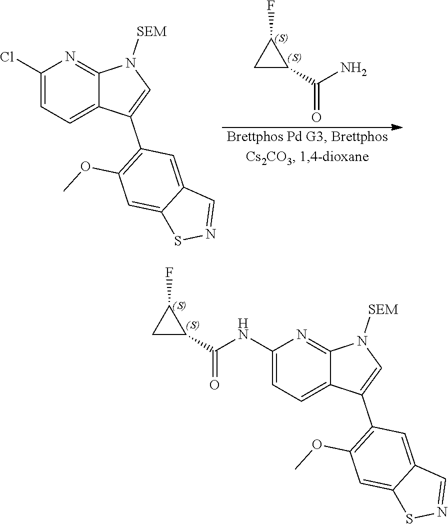

To a solution of 5-(6-chloro-1-[[2-(trimethylsilyl)ethoxy]methyl]pyrrolo[2,3-b]pyridin-3-yl)-6-methoxy-1,2-benzothiazole (200.0 mg, 0.44 mmol) in 1,4-dioxane (5.0 mL) was added (1S,2S)-2-fluorocyclopropane-1-carboxamide (230.0 mg, 2.24 mmol), Cs₂CO₃ (438.2 mg, 1.34 mmol), BrettPhos (48.1 mg, 0.09 mmol) and BrettPhos Pd G3 (40.6 mg, 0.05 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford (1S,2S)-2-fluoro-N-[3-(6-methoxy-1,2-benzothiazol-5-yl)-1-[[2-(trimethylsilyl)ethoxy]methyl]pyrrolo[2,3-b]pyridin-6-yl]cyclopropane-1-carboxamide (83.0 mg, 36%) as a yellow solid. LCMS (ESI, m/z): [M+H]⁺=513.2.

### Step 6: Synthesis of (1S,2S)-2-fluoro-N-[3-(6-methoxy-1,2-benzothiazol-5-yl)-1H-pyrrolo[2,3-b]pyridin-6-yl]cyclopropane-1-carboxamide (Compound 84)

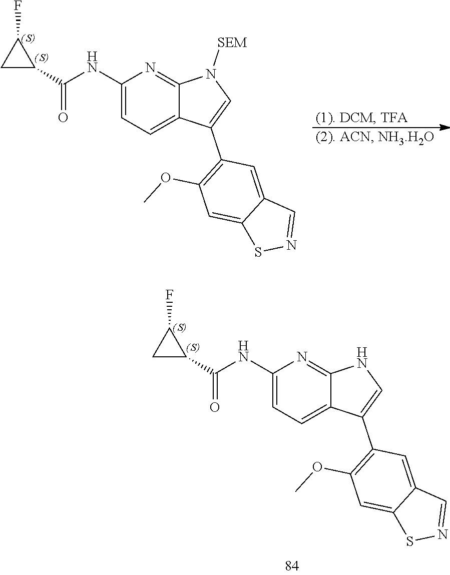

To a solution of (1S,2S)-2-fluoro-N-[3-(6-methoxy-1,2-benzothiazol-5-yl)-1-[[2-(trimethylsilyl)ethoxy]methyl]pyrrolo[2,3-b]pyridin-6-yl]cyclopropane-1-carboxamide (83.0 mg, 0.16 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in ACN (3.0 mL) was added NH₃·H₂O (3.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 24% B to 54% B in 10 min; 254 nm) to afford (1S,2S)-2-fluoro-N-[3-(6-methoxy-1,2-benzothiazol-5-yl)-1H-pyrrolo[2,3-b]pyridin-6-yl]cyclopropane-1-carboxamide (4.0 mg, 6%) as a white solid. LCMS (ESI, m/z): [M+H]⁺=383.2. ¹H NMR (400 MHz, DMSO-d₆): δ 11.68 (d, J=2.0 Hz, 1H), 10.66 (s, 1H), 9.00 (d, J=0.8 Hz, 1H), 8.31 (s, 1H), 8.10 (d, J=8.8 Hz, 1H), 7.94-7.89 (m, 2H), 7.68 (d, J=2.0 Hz, 1H), 5.01-4.84 (m, 1H), 3.94 (s, 3H), 2.30-2.23 (m, 1H), 1.69-1.63 (m, 1H), 1.20-1.10 (m, 1H).

### Example S85. Compound 85

### Step 1: Synthesis of 5-bromo-6-methoxy-1,3-benzothiazol-2-amine

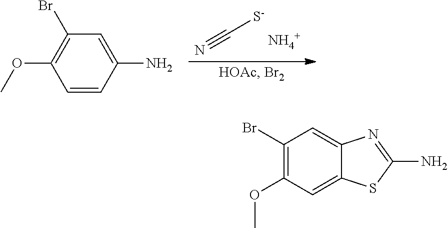

To a solution of 3-bromo-4-methoxyaniline (4.0 g, 19.80 mmol) in HOAc (30.0 mL) was added ammonium thiocyanate (7.6 g, 99.84 mmol) at room temperature under N₂. The resulting mixture was stirred at room temperature for 1 h. Then Br₂ (1.2 mL) was added dropwise to the mixture at 0° C. The resulting mixture was stirred at 0° C. for another 2 h. After the reaction was completed, the reaction mixture was quenched with sat. NH₄Cl (aq.) at 0° C. The pH value of the mixture was adjusted to 12 with NH₃·H₂O (aq.). The resulting mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (20/80, v/v) to afford 5-bromo-6-methoxy-1,3-benzothiazol-2-amine (3.2 g, 62%) as an off white solid. LCMS (ESI, m/z): [M+H]⁺=258.9.

### Step 2: Synthesis of 5-bromo-6-methoxy-1,3-benzothiazole

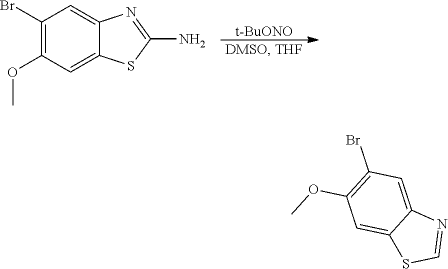

To a solution of 5-bromo-6-methoxy-1,3-benzothiazol-2-amine (2.7 g, 10.42 mmol) in THE (10.0 mL) was added DMSO (90.0 mg, 1.15 mmol) and t-BuONO (1.8 g, 17.45 mmol) at room temperature. The resulting mixture was stirred at 30° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 5-bromo-6-methoxy-1,3-benzothiazole (2.3 g, 90%) as a white solid. LCMS (ESI, m/z): [M+H]⁺=243.9.

### Step 3: Synthesis of 6-methoxy-5-(4,4,5,5-tetramethyl-1,3,2-dioxaborolan-2-yl)-benzo[d]thiazole

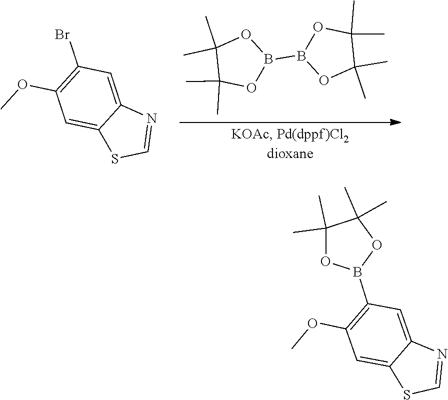

To a solution of 5-bromo-6-methoxy-1,3-benzothiazole (500.0 mg, 2.04 mmol) in dioxane (5.0 mL) was added bis(pinacolato)diboron (1.04 g, 4.09 mmol), KOAc (603.1 mg, 6.14 mmol) and Pd(dppf)Cl₂ (149.8 mg, 0.20 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash chromatography with MeOH/H₂O (1/1, v/v) to afford 6-methoxy-5-(4,4,5,5-tetramethyl-1,3,2-dioxaborolan-2-yl)-benzothiazole (300.0 mg, 50%) as a white solid. LCMS (ESI, m/z): [M+H]⁺=292.1.

### Step 4: Synthesis of 5-(6-chloro-1-[[2-(trimethylsilyl)ethoxy]methyl]pyrrolo[2,3-b]pyridin-3-yl)-6-methoxy-1,3-benzothiazole

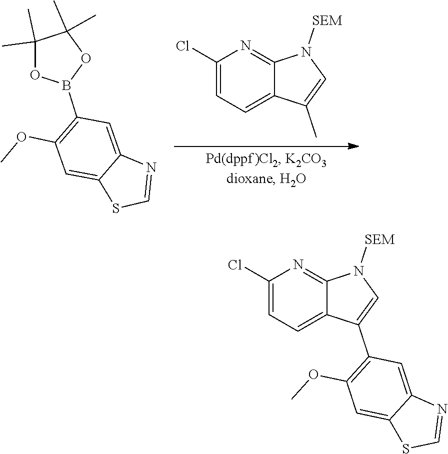 [SMILES: COc1cc2scnc2cc1B1OC(C)(C)C(C)(C)O1 ; Cc1cn(COCC[Si](C)(C)C)c2nc(Cl)ccc12 ; COc1cc2scnc2cc1-c1cn(COCC[Si](C)(C)C)c2nc(Cl)ccc12]

To a solution of 6-methoxy-5-(4,4,5,5-tetramethyl-1,3,2-dioxaborolan-2-yl)-1,3-benzothiazole (300.0 mg, 1.03 mmol) in dioxane/H₂O (5.0/1.0 mL) was added 3-bromo-6-chloro-1-[[2-(trimethylsilyl)ethoxy]methyl]pyrrolo[2,3-b]pyridine (447.2 mg, 1.23 mmol), K₂CO₃ (599.5 mg, 4.33 mmol) and Pd(dppf)Cl₂ (105.5 mg, 0.14 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 5-(6-chloro-1-[[2-(trimethylsilyl)ethoxy]methyl]pyrrolo[2,3-b]pyridin-3-yl)-6-methoxy-1,3-benzothiazole (240.0 mg, 52%) as a white solid. LCMS (ESI, m/z): [M+H]⁺=446.1.

### Step 5: Synthesis of (1S,2S)-2-fluoro-N-[3-(6-methoxy-1,3-benzothiazol-5-yl)-1-[[2-(trimethylsilyl)ethoxy]methyl]pyrrolo[2,3-b]pyridin-6-yl]cyclopropane-1-carboxamide

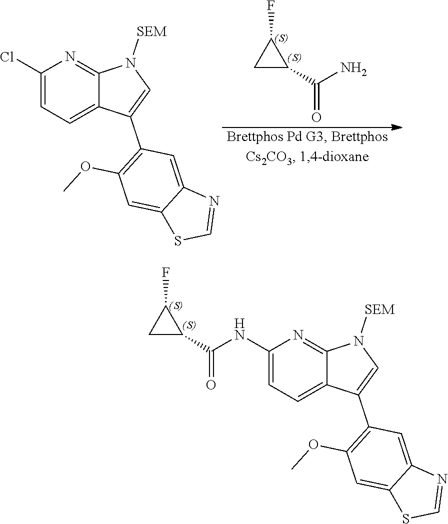

To a solution of 5-(6-chloro-1-[[2-(trimethylsilyl)ethoxy]methyl]pyrrolo[2,3-b]pyridin-3-yl)-6-methoxy-1,3-benzothiazole (200.0 mg, 0.44 mmol) in 1,4-dioxane (5.0 mL) was added (1S,2S)-2-fluorocyclopropane-1-carboxamide (140.0 mg, 1.35 mmol), BrettPhos (48.1 mg, 0.09 mmol), Cs₂CO₃ (438.2 mg, 1.34 mmol) and BrettPhos Pd G3 (40.6 mg, 0.05 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford (1S,2S)-2-fluoro-N-[3-(6-methoxy-1,3-benzothiazol-5-yl)-1-[[2-(trimethylsilyl)ethoxy]methyl]pyrrolo[2,3-b]pyridin-6-yl]cyclopropane-1-carboxamide (150.0 mg, 65%) as a white solid. LCMS (ESI, m/z): [M+H]⁺=513.2.

### Step 6: Synthesis of (1S,2S)-2-fluoro-N-[3-(6-methoxy-1,3-benzothiazol-5-yl)-1H-pyrrolo[2,3-b]pyridin-6-yl]cyclopropane-1-carboxamide (Compound 85)

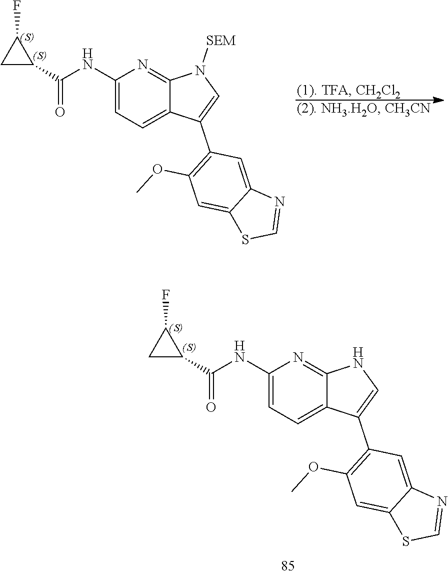

To a solution of (1S,2S)-2-fluoro-N-[3-(6-methoxy-1,3-benzothiazol-5-yl)-1-[[2-(trimethylsilyl)ethoxy]methyl]pyrrolo[2,3-b]pyridin-6-yl]cyclopropane-1-carboxamide (130.0 mg, 0.25 mmol) in CH₂Cl₂ (3.0 mL) was added TFA (3.0 mL) at room temperature. The resulting mixture was stirred at room temperature for 3 h. The resulting mixture was concentrated under reduced pressure. The residue was re-dissolved in CH₃CN (3.0 mL) and NH₃·H₂O (3.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 10 min; 254 nm) to afford (1S,2S)-2-fluoro-N-[3-(6-methoxy-1,3-benzothiazol-5-yl)-1H-pyrrolo[2,3-b]pyridin-6-yl]cyclopropane-1-carboxamide (36.4 mg, 38%) as a white solid. LCMS (ESI, m/z): [M+H]⁺=383.2. ¹H NMR (300 MHz, DMSO-d₆): δ 11.66 (s, 1H), 10.64 (s, 1H), 9.22 (s, 1H), 8.14 (s, 1H), 8.01 (d, J=8.4 Hz, 1H), 7.93-7.87 (m, 2H), 7.66 (d, J=2.7 Hz, 1H), 5.06-4.79 (m, 1H), 3.90 (s, 3H), 2.26-2.20 (m, 1H), 1.73-1.63 (m, 1H), 1.29-1.09 (m, 1H).

### Example S86. Compound 86

### Step 1: Synthesis of N-((4-bromo-3-methoxyphenyl)carbamothioyl)benzamide

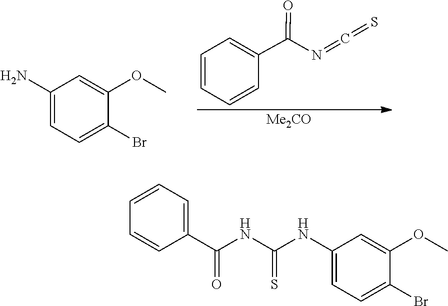

To a solution of 4-bromo-3-methoxyaniline (2.0 g, 9.90 mmol) in Me₂CO (50.0 mL) was added benzoyl isothiocyanate (1.6 g, 9.90 mmol) at room temperature. The resulting mixture was stirred at 60° C. for 30 min. After the reaction was completed, the reaction mixture was cooled to room temperature and filtered. The solid was collected and dried to afford N-((4-bromo-3-methoxyphenyl)carbamothioyl)benzamide (3.1 g, crude) as a white solid. LCMS (ESI, m/z): [M+H]⁺=365.0.

### Step 2: Synthesis of 1-(4-bromo-3-methoxyphenyl)thiourea

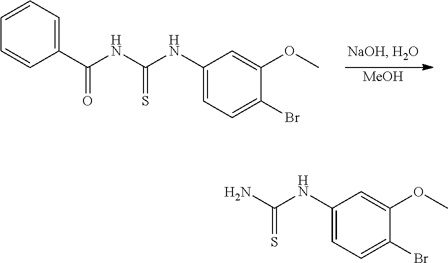 [SMILES: "MeOH" ; COc1cc(NC(=S)NC(=O)c2ccccc2)ccc1Br ; COc1cc(NC(N)=S)ccc1Br]

To a solution of N-((4-bromo-3-methoxyphenyl)carbamothioyl)benzamide (3.1 g, 8.49 mmol) in MeOH/H₂O (30.0/30.0 mL) was added NaOH (0.4 g, 9.34 mmol) at room temperature. The resulting mixture was stirred at 65° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 1-(4-bromo-3-methoxyphenyl)thiourea (1.9 g, crude) as a white solid. LCMS (ESI, m/z): [M+H]⁺=261.0.

### Step 3: Synthesis of 6-bromo-5-methoxybenzo[d]thiazol-2-amine

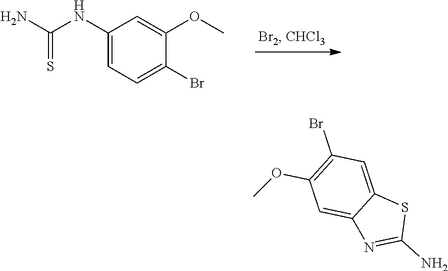

To a solution of 1-(4-bromo-3-methoxyphenyl)thiourea (1.9 g, 7.28 mmol) in CHCl₃ (30.0 mL) was added Br₂ (1.4 g, 8.73 mmol) dropwise at 0° C. The resulting mixture was stirred at room temperature for 16 h. After the reaction was completed, the mixture was filtered. The solid was collected and dried to afford 6-bromo-5-methoxybenzo[d]thiazol-2-amine (1.8 g, crude) as a yellow solid. LCMS (ESI, m/z): [M+H]⁺=258.9.

### Step 4: Synthesis of 6-bromo-5-methoxybenzo[d]thiazole

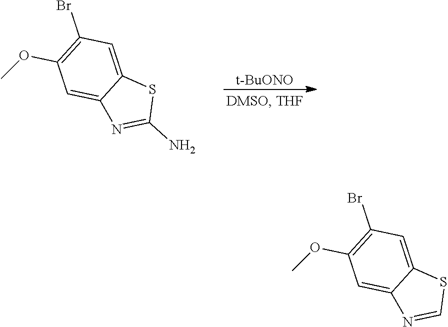

To a solution of 6-bromo-5-methoxybenzo[d]thiazol-2-amine (1.8 g, 6.95 mmol) in THE (30.0 mL) was added t-BuONO (1.1 g, 10.42 mmol) and DMSO (43.4 mg, 0.56 mmol) at room temperature. The resulting mixture was stirred at room temperature for 2 h. After the reaction was completed, the resulting mixture was concentrated under reduced pressure. The residue was purified by flash column chromatography with petroleum ether/ethyl acetate (1/1, v/v) to afford 6-bromo-5-methoxybenzo[d]thiazole (470.0 mg, 27%) as a yellow solid. LCMS (ESI, m/z): [M+H]⁺=243.9.

### Step 5: Synthesis of 6-(6-chloro-1-((2-(trimethylsilyl)ethoxy)methyl)-1H-pyrrolo[2,3-b]pyridin-3-yl)-5-methoxybenzo[d]thiazole

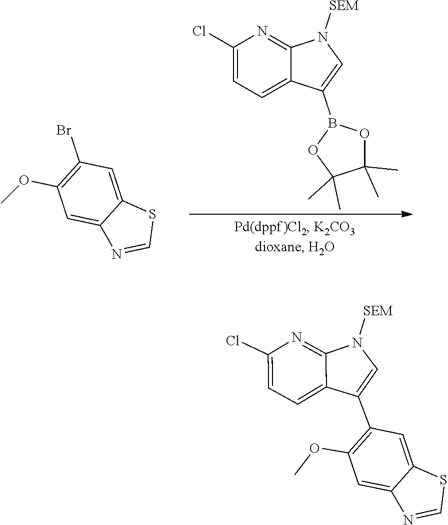 [SMILES: COc1cc2ncsc2cc1Br ; CC1(C)OB(c2cn(COCC[Si](C)(C)C)c3nc(Cl)ccc23)OC1(C)C ; COc1cc2ncsc2cc1-c1cn(COCC[Si](C)(C)C)c2nc(Cl)ccc12]

To a solution of 6-bromo-5-methoxybenzo[d]thiazole (200.0 mg, 0.82 mmol) in dioxane/H₂O (10.0/2.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (334.9 mg, 0.82 mmol), K₂CO₃ (339.7 mg, 2.46 mmol) and Pd(dppf)Cl₂ (60.0 mg, 0.08 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-(6-chloro-1-((2-(trimethylsilyl)ethoxy)methyl)-1H-pyrrolo[2,3-b]pyridin-3-yl)-5-methoxybenzo[d]thiazole (210.0 mg, 57%) as a yellow solid. LCMS (ESI, m/z): [M+H]⁺=446.1.

### Step 6: Synthesis of (1S,2S)-2-fluoro-N-(3-(5-methoxybenzo[d]thiazol-6-yl)-1-((2-(trimethylsilyl)ethoxy)methyl)-1H-pyrrolo[2,3-b]pyridin-6-yl)cyclopropane-1-carboxamide

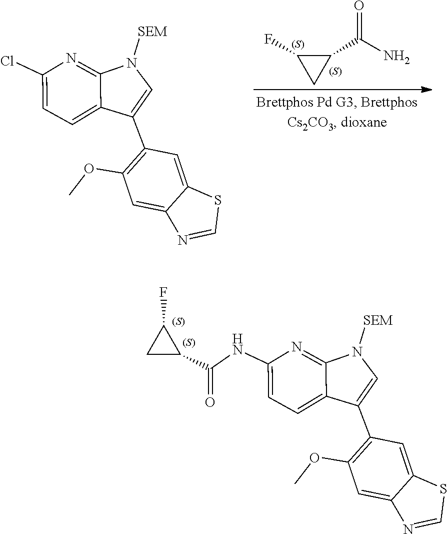

To a solution of 6-(6-chloro-1-((2-(trimethylsilyl)ethoxy)methyl)-1H-pyrrolo[2,3-b]pyridin-3-yl)-5-methoxybenzo[d]thiazole (190.0 mg, 0.43 mmol) in dioxane (10.0 mL) was added (1S,2S)-2-fluorocyclopropane-1-carboxamide (219.6 mg, 2.13 mmol), Cs₂CO₃ (416.4 mg, 1.28 mmol), Brettphos (45.7 mg, 0.09 mmol) and BrettPhos Pd G3 (38.6 mg, 0.04 mmol) at room temperature under N₂. The resulting mixture was stirred at 100° C. for 4 h under N₂. After the reaction was completed, the reaction mixture was filtered. The filtrate was concentrated under reduced pressure. The residue was purified by flash column chromatography with petroleum ether/ethyl acetate (1/1, v/v) to afford (1S,2S)-2-fluoro-N-(3-(5-methoxybenzo[d]thiazol-6-yl)-1-((2-(trimethylsilyl)ethoxy)methyl)-1H-pyrrolo[2,3-b]pyridin-6-yl)cyclopropane-1-carboxamide (80.0 mg, 36%) as a yellow solid. LCMS (ESI, m/z): [M+H]⁺=513.2.

### Step 7: Synthesis of (1S,2S)-2-fluoro-N-(3-(5-methoxybenzo[d]thiazol-6-yl)-1H-pyrrolo[2,3-b]pyridin-6-yl)cyclopropane-1-carboxamide (Compound 86)

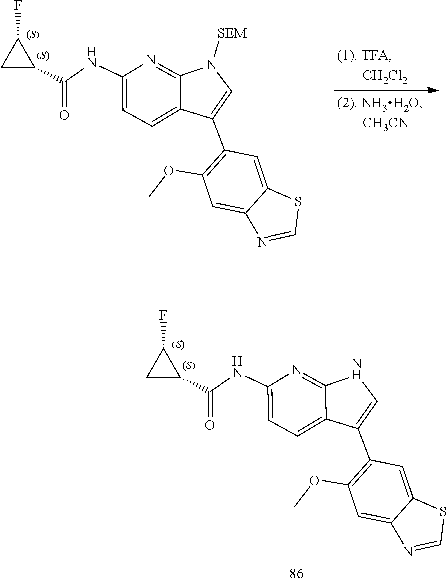

To a solution of (1S,2S)-2-fluoro-N-(3-(5-methoxybenzo[d]thiazol-6-yl)-1-((2-(trimethylsilyl)ethoxy)methyl)-1H-pyrrolo[2,3-b]pyridin-6-yl)cyclopropane-1-carboxamide (60.0 mg, 0.12 mmol) in CH₂Cl₂ (3.0 mL) was added TFA (3.0 mL) at room temperature. The resulting mixture was stirred at room temperature for 3 h. The resulting mixture was concentrated under reduced pressure. The residue was dissolved in CH₃CN (5.0 mL) and NH₃·H₂O (5.0 mL). The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 30% to 40% in 9 min; 254 nm) to afford (1S,2S)-2-fluoro-N-(3-(5-methoxybenzo[d]thiazol-6-yl)-1H-pyrrolo[2,3-b]pyridin-6-yl)cyclopropane-1-carboxamide (22.1 mg, 49%) as a white solid. LCMS (ESI, m/z): [M+H]⁺=383.1. ¹H NMR (300 MHz, DMSO-d₆): δ 11.67 (s, 1H), 10.66 (s, 1H), 9.33 (s, 1H), 8.28 (s, 1H), 8.10 (d, J=8.7 Hz, 1H), 7.92 (d, J=8.7 Hz, 1H), 7.76 (s, 1H), 7.69 (d, J=2.1 Hz, 1H), 5.07-4.80 (m, 1H), 3.94 (s, 3H), 2.29-2.20 (m, 1H), 1.73-1.62 (m, 1H), 1.22-1.15 (m, 1H).

### Example S87. Compound 87

### Step 1: Synthesis of 1-bromo-5-fluoro-2-methoxy-4-nitrobenzene

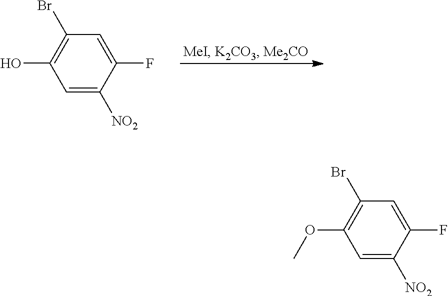

A mixture of 2-bromo-4-fluoro-5-nitrophenol (3.0 g, 12.71 mmol), K₂CO₃ (3.5 g, 25.42 mmol) and MeI (3.6 g, 25.42 mmol) in acetone (20.0 mL) was stirred at 50° C. for 16 h. After the reaction was completed, the resulting mixture was filtered. The filtrate was concentrated under reduced pressure to afford 1-bromo-5-fluoro-2-methoxy-4-nitrobenzene (6.0 g, crude) as a yellow solid.

### Step 2: Synthesis of 5-bromo-4-methoxy-2-nitrophenol

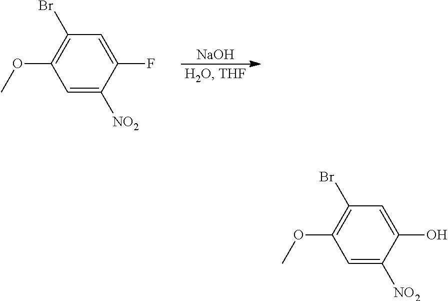

To a solution of 1-bromo-5-fluoro-2-methoxy-4-nitrobenzene (3.2 g, 12.79 mmol) in THF/H₂O (10.0/10.0 mL) was added NaOH (767.8 mg, 19.19 mmol). The resulting mixture was stirred at 60° C. for 16 h. After the reaction was completed, the PH value of the mixture was adjusted to 6 with HCl (aq.). The resulting mixture was extracted with dichloromethane. The combined organic layer was washed with brine, dried over anhydrous sodium sulfate and filtered. The filtrate was concentrated under vacuum to afford 5-bromo-4-methoxy-2-nitrophenol (2.5 g, crude) as a yellow solid. LCMS (ESI, m/z): [M+H]⁺=247.9.

### Step 3: Synthesis of 2-amino-5-bromo-4-methoxyphenol

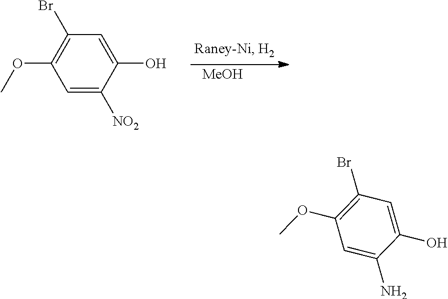

To a solution of 4-bromo-5-methoxy-2-nitrophenol (400.0 mg, 1.61 mmol) in MeOH (10.0 mL) was added Raney Ni (400.6 mg, 4.67 mmol). The mixture was stirred at room temperature for 2 h under H₂. After the reaction was completed, the reaction mixture was filtered. The filtrate was concentrated under vacuum to afford 2-amino-5-bromo-4-methoxyphenol (400.0 mg, crude) as a brown solid. LCMS (ESI, m/z): [M+H]⁺=218.0.

### Step 4: Synthesis of 6-bromo-5-methoxy-1,3-benzoxazole

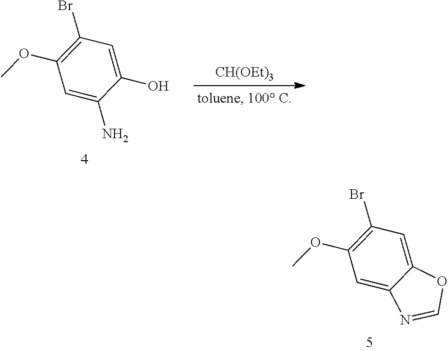

A mixture of 2-amino-4-bromo-5-methoxyphenol (400.0 mg, 1.83 mmol) and triethyl orthoformate (399.6 mg, 2.69 mmol) in toluene (5.0 mL) was stirred at 100° C. for 16 h. The resulting mixture was concentrated under vacuum. The residue was diluted with ethyl acetate. The organic layer was washed with brine, dried over anhydrous sodium sulfate and filtered. The filtrate was concentrated under vacuum. The residue was purified by flash column chromatography with petroleum ether/ethyl acetate (70/30, v/v) to afford 6-bromo-5-methoxybenzo[d]oxazole (300.0 mg, 72%) as a yellow solid. LCMS (ESI, m/z): [M+H]⁺=228.0.

### Step 5: Synthesis of 6-(6-chloro-1-[[2-(trimethylsilyl)ethoxy]methyl]pyrrolo[2,3-b]pyridin-3-yl)-5-methoxy-1,3-benzoxazole

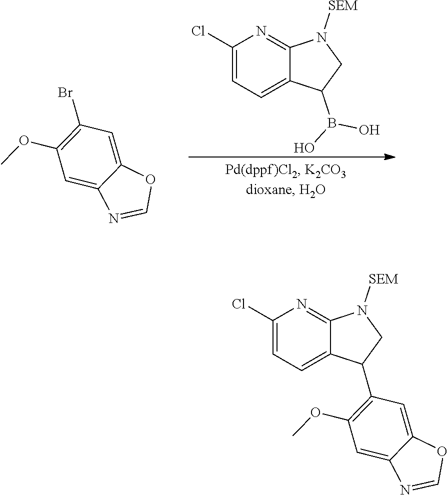 [SMILES: COc1cc2ncoc2cc1Br ; C[Si](C)(C)CCOCN1CC(B(O)O)c2ccc(Cl)nc21 ; COc1cc2ncoc2cc1C1CN(COCC[Si](C)(C)C)c2nc(Cl)ccc21]

To a mixture of 6-bromo-5-methoxy-1,3-benzoxazole (200.0 mg, 0.87 mmol) and 6-chloro-1-[[2-(trimethylsilyl)ethoxy]methyl]pyrrolo[2,3-b]pyridin-3-ylboronic acid (315.1 mg, 0.96 mmol) in dioxane/H₂O (5.0/0.5 mL) was added Pd(dppf)Cl₂ (71.6 mg, 0.09 mmol) and K₂CO₃ (363.6 mg, 2.63 mmol). The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash column chromatography with H₂O/ACN (50/50, v/v) to afford 6-(6-chloro-1-[[2-(trimethylsilyl)ethoxy]methyl]pyrrolo[2,3-b]pyridin-3-yl)-5-methoxy-1,3-benzoxazole (180.0 mg, 47%) as a brown yellow oil. LCMS (ESI, m/z): [M+H]⁺=430.1.

### Step 6: Synthesis of (1S,2S)-2-fluoro-N-[3-(5-methoxy-1,3-benzoxazol-6-yl)-1-[[2-(trimethylsilyl)ethoxy]methyl]pyrrolo[2,3-b]pyridin-6-yl]cyclopropane-1-carboxamide

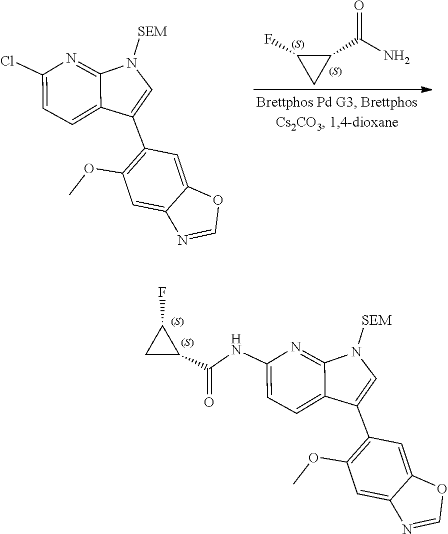

To a mixture of 6-(6-chloro-1-[[2-(trimethylsilyl)ethoxy]methyl]pyrrolo[2,3-b]pyridin-3-yl)-5-methoxy-1,3-benzoxazole (90.0 mg, 0.21 mmol) and (1S,2S)-2-fluorocyclopropane-1-carboxamide (107.9 mg, 1.05 mmol) in 1,4-dioxane (5.0 mL) was added BrettPhos Pd G3 (37.9 mg, 0.04 mmol), Cs₂CO₃ (204.6 mg, 0.63 mmol) and BrettPhos (44.9 mg, 0.08 mmol). The resulting mixture was stirred at 100° C. for 16 h under N₂. The reaction mixture was diluted with water and extracted with ethyl acetate. The combined organic layer was washed with brine, dried over anhydrous sodium sulfate and filtered. The filtrate was concentrated under vacuum. The residue was purified by reverse phase flash column chromatography with H₂O/ACN (36/64, v/v) to afford (1S,2S)-2-fluoro-N-[3-(5-methoxy-1,3-benzoxazol-6-yl)-1-[[2-(trimethylsilyl)ethoxy]methyl]pyrrolo[2,3-b]pyridin-6-yl]cyclopropane-1-carboxamide (35.0 mg, 33%) as a colorless oil. LCMS (ESI, m/z): [M+H]⁺=497.2.

### Step 7: Synthesis of (1S,2S)-2-fluoro-N-(3-(5-methoxybenzo[d]oxazol-6-yl)-1H-pyrrolo[2,3-b]pyridin-6-yl)cyclopropane-1-carboxamide (Compound 87)

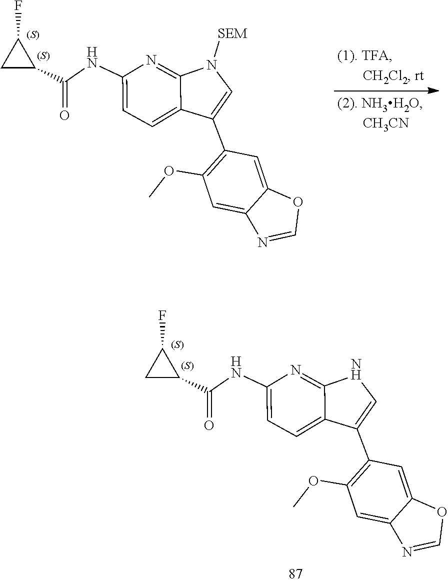

A mixture of (1S,2S)-2-fluoro-N-[3-(5-methoxy-1,3-benzoxazol-6-yl)-1-[[2-(trimethylsilyl)ethoxy] methyl]pyrrolo [2,3-b]pyridin-6-yl] cyclopropane-1-carboxamide (35.0 mg, 0.07 mmol) and TFA (2.0 mL) in DCM (2.0 mL) was stirred at room temperature for 2 h. The resulting mixture was concentrated under reduced pressure. To the above residue was added ACN (2.0 mL) and NH₃·H₂O (2.0 mL). The resulting mixture was stirred at room temperature for another 4 h. After the reaction was completed, the resulting mixture was concentrated under vacuum. The residue was purified by Prep-HPLC with the following conditions: Column: Xselect CSH OBD Column 30×150 mm, 5 um; Mobile Phase A: Water, Mobile Phase B: ACN; Flow rate: 60 mL/min; Gradient: 5% B to 80% B in 7 min; 254/220 nm to afford (1S,2S)-2-fluoro-N-(3-(5-methoxybenzo[d]oxazol-6-yl)-1H-pyrrolo[2,3-b]pyridin-6-yl)cyclopropane-1-carboxamide (3.9 mg, 15%) as a white solid. LCMS (ESI, m/z): [M+H]⁺=367.1. ¹H NMR (300 MHz, DMSO-d₆): δ 11.64 (s, 1H), 10.63 (s, 1H), 8.67 (s, 1H), 8.01 (d, J=8.7 Hz, 1H), 7.93-7.88 (m, 1H), 7.84 (s, 1H), 7.62 (d, J=2.4 Hz, 1H), 7.48 (s, 1H), 5.05-4.77 (m, 1H), 3.86 (s, 3H), 2.29-2.20 (m, 1H), 1.71-1.57 (m, 1H), 1.22-1.08 (m, 1H).

### Example S88. Compound 88

### Step 1: Synthesis of 4-bromo-5-methoxy-2-nitrophenol

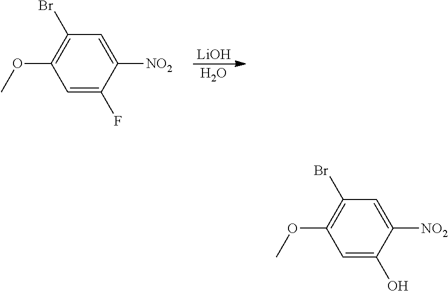

To a solution of 1-bromo-4-fluoro-2-methoxy-5-nitrobenzene (500.0 mg, 2.00 mmol) in H₂O (20.0 mL) was added lithium hydroxide (239.4 mg, 9.99 mmol). The resulting mixture was stirred at 100° C. for 15 min. After the reaction was completed, the resulting mixture was diluted with water. The pH value of the mixture was adjusted to 5 with 1N HCl. The mixture was extracted with ethyl acetate. The combined organic layer was dried over anhydrous sodium sulfate and filtered. The filtrate was concentrated under vacuum to afford 4-bromo-5-methoxy-2-nitrophenol (480.0 mg, crude) as a yellow solid.

### Step 2: Synthesis of 2-amino-4-bromo-5-methoxyphenol

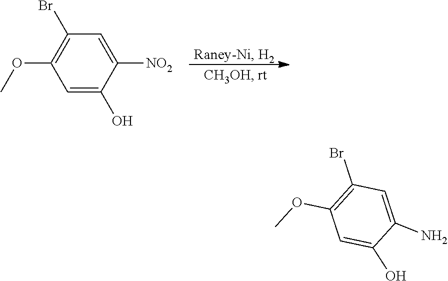

To a solution of 4-bromo-5-methoxy-2-nitrophenol (400.0 mg, 1.61 mmol) in MeOH (10.0 mL) was added Raney Ni (400.6 mg, 4.67 mmol). The resulting mixture was stirred at room temperature for 2 h under H₂. After the reaction was completed, the reaction mixture was filtered. The filtrate was concentrated under vacuum to afford 2-amino-4-bromo-5-methoxyphenol (400.0 mg, crude) as a brown yellow solid. LCMS (ESI, m/z): [M+H]⁺=218.0.

### Step 3: Synthesis of 5-bromo-6-methoxybenzo[d]oxazole

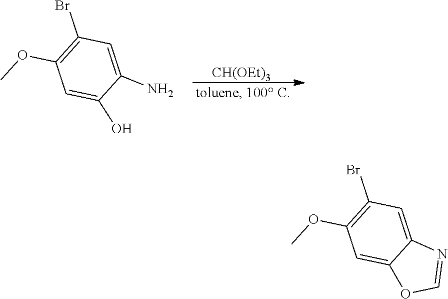

A mixture of 2-amino-4-bromo-5-methoxyphenol (400.0 mg, 1.83 mmol) and triethyl orthoformate (399.6 mg, 2.69 mmol) in toluene (5.0 mL) was stirred at 100° C. for 16 h. After the reaction was completed, the resulting mixture was concentrated under vacuum. The residue was diluted with ethyl acetate. The organic layer was washed with brine, dried over anhydrous sodium sulfate and filtered. The filtrate was concentrated under vacuum. The residue was purified by flash column chromatography with petroleum ether/ethyl acetate (70/30, v/v) to afford 5-bromo-6-methoxybenzo[d]oxazole (300.0 mg, 72%) as a brown solid. LCMS (ESI, m/z): [M+H]⁺=228.0.

### Step 4: Synthesis of 5-(6-chloro-1-((2-(trimethylsilyl)ethoxy)methyl)-1H-pyrrolo[2,3-b]pyridin-3-yl)-6-methoxybenzo[d]oxazole

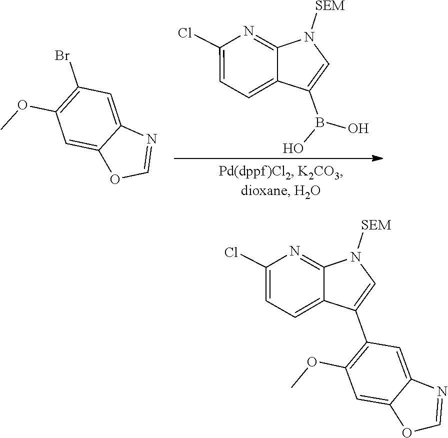 [SMILES: COc1cc2ocnc2cc1Br ; C[Si](C)(C)CCOCn1cc(B(O)O)c2ccc(Cl)nc21 ; COc1cc2ocnc2cc1-c1cn(COCC[Si](C)(C)C)c2nc(Cl)ccc12]

To a mixture of 5-bromo-6-methoxy-1,3-benzoxazole (300.0 mg, 1.31 mmol) and 6-chloro-1-((2-(trimethylsilyl)ethoxy)methyl)-1H-pyrrolo[2,3-b]pyridin-3-ylboronic acid (537.8 mg, 1.31 mmol) in 1,4-dioxane (5.0 mL) and H₂O (0.5 mL) was added Pd(dppf)Cl₂ (192.5 mg, 0.26 mmol) and K₂CO₃ (545.4 mg, 3.94 mmol). The mixture was stirred at 80° C. for 16 h. After the reaction was completed, the reaction mixture was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford 5-(6-chloro-1-[[2-(trimethylsilyl)ethoxy]methyl]pyrrolo[2,3-b]pyridin-3-yl)-6-methoxy-1,3-benzoxazole (280.0 mg, 49%) as a brown yellow oil. LCMS (ESI, m/z): [M+H]⁺=430.1.

### Step 5: Synthesis of (1S,2S)-2-fluoro-N-(3-(6-methoxybenzo[d]oxazol-5-yl)-1-((2-(trimethylsilyl)ethoxy)methyl)-1H-pyrrolo[2,3-b]pyridin-6-yl)cyclopropane-1-carboxamide

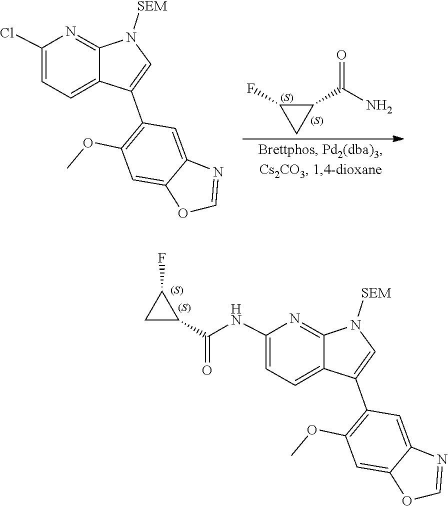

To a mixture of 5-(6-chloro-1-[[2-(trimethylsilyl)ethoxy]methyl]pyrrolo[2,3-b]pyridin-3-yl)-6-methoxy-1,3-benzoxazole (240.0 mg, 0.55 mmol) and (1S,2S)-2-fluorocyclopropane-1-carboxamide (575.4 mg, 5.58 mmol) in 1,4-dioxane (5.0 mL) was added BrettPhos (119.8 mg, 0.22 mmol), Pd₂(dba)₃ (102.2 mg, 0.11 mmol) and Cs₂CO₃ (545.5 mg, 1.67 mmol). The mixture was stirred at 100° C. for 16 h under N₂. After the reaction was completed, the reaction mixture was diluted with water and extracted with ethyl acetate. The combined organic layer was washed with brine, dried over anhydrous sodium sulfate and filtered. The filtrate was concentrated under vacuum. The residue was purified by reverse phase flash chromatography with ACN/water (30/70, v/v) to afford (1S,2S)-2-fluoro-N-[3-(6-methoxy-1,3-benzoxazol-5-yl)-1-[[2-(trimethylsilyl)ethoxy]methyl]pyrrolo[2,3-b]pyridin-6-yl]cyclopropane-1-carboxamide (77.0 mg, 27%) as a yellow solid. LCMS (ESI, m/z): [M+H]⁺=497.2

### Step 6: Synthesis of (1S,2S)-2-fluoro-N-(3-(6-methoxybenzo[d]oxazol-5-yl)-1H-pyrrolo[2,3-b]pyridin-6-yl)cyclopropane-1-carboxamide (Compound 88)

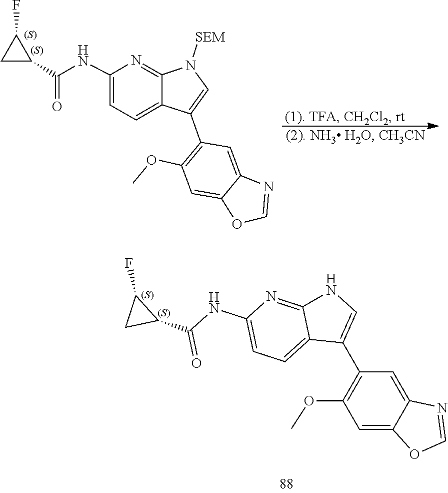

A mixture of (1S,2S)-2-fluoro-N-[3-(6-methoxy-1,3-benzoxazol-5-yl)-1-[[2-(trimethylsilyl)ethoxy]methyl]pyrrolo[2,3-b]pyridin-6-yl]cyclopropane-1-carboxamide (67.0 mg, 0.14 mmol) and TFA (1.0 mL) in DCM (1.0 mL) was stirred at room temperature for 2 h. The resulting mixture was concentrated under reduced pressure. To the above residue was added ACN (1.00 mL) and NH₃·H₂O (1.00 mL). The resulting mixture was stirred at room temperature for another 4 h. After the reaction was completed, the resulting mixture was concentrated under vacuum. The residue was purified by Prep-HPLC with the following conditions: Column: Xselect CSH OBD Column 30×150 mm 5 um; Mobile Phase A: Water, Mobile Phase B: ACN; Flow rate: 60 mL/min; Gradient: 5% B to 80% B in 7 min; 254/220 nm to afford (1S,2S)-2-fluoro-N-[3-(6-methoxy-1,3-benzoxazol-5-yl)-1H-pyrrolo[2,3-b]pyridin-6-yl]cyclopropane-1-carboxamide (14.7 mg, 29%) as a white solid. LCMS (ESI, m/z): [M+H]⁺=367.1 ¹H NMR (300 MHz, DMSO-d₆): δ 11.59 (s, 1H), 10.62 (s, 1H), 8.62 (s, 1H), 7.94-7.89 (m, 2H), 7.80 (s, 1H), 7.56-7.54 (m, 2H), 5.01-4.80 (m, 1H), 3.86 (s, 3H), 2.28-2.17 (m, 1H), 1.86-1.62 (m, 1H), 1.18-0.83 (m, 1H).

### Example S89. Compound 89

### Step 1: Synthesis of 3-bromo-6-chloro-2-methyl-1-((2-(trimethylsilyl)ethoxy)methyl)-1H-pyrrolo[2,3-b]pyridine

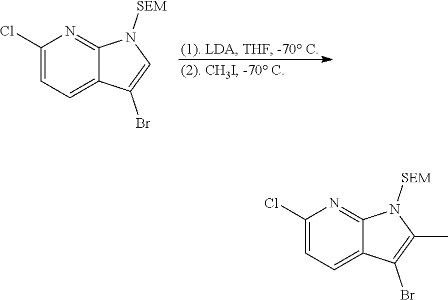 [SMILES: C[Si](C)(C)CCOCn1cc(Br)c2ccc(Cl)nc21 ; Cc1c(Br)c2ccc(Cl)nc2n1COCC[Si](C)(C)C]

To a solution of 3-bromo-6-chloro-1-((2-(trimethylsilyl)ethoxy)methyl)-1H-pyrrolo[2,3-b]pyridine (500.0 mg, 1.38 mmol) in THF (10.0 mL) was added dropwise LDA (2.1 mL) at −70° C. under N₂. The resulting mixture was stirred at −70° C. for 2 h. Then CH₃I (588.6 mg, 4.15 mmol) was added dropwise to the mixture at −70° C. under N₂. The resulting mixture was stirred at −70° C. for another 2 h. After the reaction was completed, the reaction was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 3-bromo-6-chloro-2-methyl-1-((2-(trimethylsilyl)ethoxy)methyl)-1H-pyrrolo[2,3-b]pyridine (320.0 mg, 61%) as a yellow oil. LCMS (ESI, m/z): [M+H]⁺=375.0.

### Step 2: Synthesis of 6-chloro-3-(2-methoxypyridin-3-yl)-2-methyl-1-((2-(trimethylsilyl)ethoxy)methyl)-1H-pyrrolo[2,3-b]pyridine

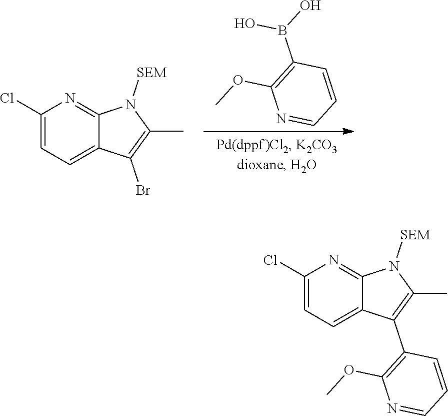

To a solution of 3-bromo-6-chloro-2-methyl-1-((2-(trimethylsilyl)ethoxy)methyl)-1H-pyrrolo[2,3-b]pyridine (300.0 mg, 0.80 mmol) in 1,4-dioxane/H₂O (5.0/1.0 mL) was added (2-methoxypyridin-3-yl)boronic acid (122.1 mg, 0.80 mmol), K₂CO₃ (331.0 mg, 2.40 mmol) and Pd(dppf)Cl₂ (64.3 mg, 0.09 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 6-chloro-3-(2-methoxypyridin-3-yl)-2-methyl-1-((2-(trimethylsilyl)ethoxy)methyl)-1H-pyrrolo[2,3-b]pyridine (190.0 mg, 38%) as a yellow oil. LCMS (ESI, m/z): [M+H]⁺=404.1.

### Step 3: Synthesis of (1S,2S)-2-fluoro-N-(3-(2-methoxypyridin-3-yl)-2-methyl-1-((2-(trimethylsilyl)ethoxy)methyl)-1H-pyrrolo[2,3-b]pyridin-6-yl)cyclopropane-1-carboxamide

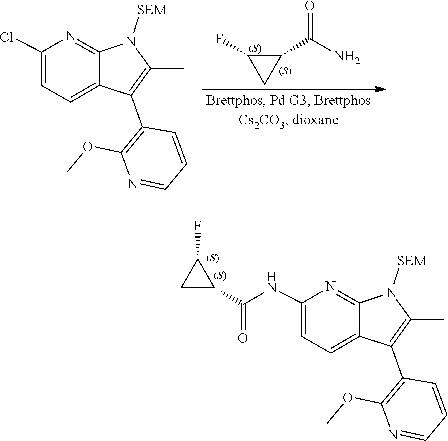

To a solution of 6-chloro-3-(2-methoxypyridin-3-yl)-2-methyl-1-((2-(trimethylsilyl)ethoxy)methyl)-1H-pyrrolo[2,3-b]pyridine (170.0 mg, 0.42 mmol) in dioxane (5.0 mL) was added (1S,2S)-2-fluorocyclopropane-1-carboxamide (216.9 mg, 2.10 mmol), Cs₂CO₃ (411.9 mg, 1.26 mmol), Brettphos (45.2 mg, 0.08 mmol) and Brettphos Pd G3 (38.2 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (77/23, v/v) to afford (1S,2S)-2-fluoro-N-(3-(2-methoxypyridin-3-yl)-2-methyl-1-((2-(trimethylsilyl)ethoxy)methyl)-1H-pyrrolo[2,3-b]pyridin-6-yl)cyclopropane-1-carboxamide (80.0 mg, 12%) as an off-white solid. LCMS (ESI, m/z): [M+H]⁺=471.2.

### Step 4: Synthesis of (1S,2S)-2-fluoro-N-(3-(2-methoxypyridin-3-yl)-2-methyl-1H-pyrrolo[2,3-b]pyridin-6-yl)cyclopropane-1-carboxamide (Compound 89)

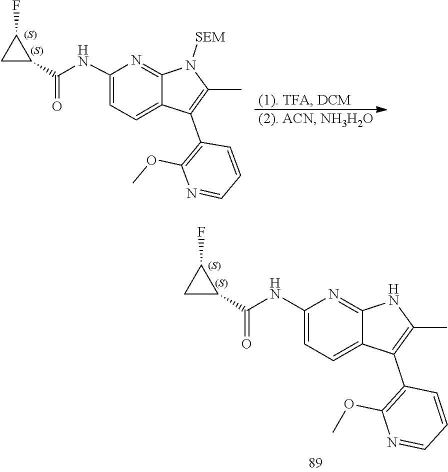

To a solution of (1S,2S)-2-fluoro-N-(3-(2-methoxypyridin-3-yl)-2-methyl-1-((2-(trimethylsilyl)ethoxy)methyl)-1H-pyrrolo[2,3-b]pyridin-6-yl)cyclopropane-1-carboxamide (80.0 mg, 0.17 mmol) in DCM (1.5 mL) was added TFA (1.5 mL) at room temperature. The resulting mixture was stirred at room temperature for 2 h. The resulting mixture was concentrated under reduced pressure. To the residue in ACN (1.5 mL) was added NH₃·H₂O (1.5 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 35% B to 40% B in 13 min; 254 nm) to afford (1S,2S)-2-fluoro-N-(3-(2-methoxypyridin-3-yl)-2-methyl-1H-pyrrolo[2,3-b]pyridin-6-yl)cyclopropane-1-carboxamide (5.6 mg, 9%) as a white solid. LCMS (ESI, m/z): [M+H]⁺=341.2. ¹H NMR (300 MHz, DMSO-d₆): δ 11.45 (s, 1H), 10.56 (s, 1H), 8.17-8.15 (m, 1H), 7.81 (d, J=8.4 Hz, 1H), 7.71-7.68 (m, 1H), 7.60 (d, J=8.4 Hz, 1H), 7.11-7.07 (m, 1H), 5.03-4.78 (m, 1H), 3.86 (s, 3H), 2.32 (s, 3H), 2.26-2.15 (m, 1H), 1.71-1.58 (m, 1H), 1.18-1.09 (m, 1H).

### Example S90. Compound 90

### Step 1: Synthesis of 5-(6-chloro-1-[[2-(trimethylsilyl)ethoxy]methyl]pyrazolo[3,4-b]pyridin-3-yl)-4-methoxy-1-[[2-(trimethylsilyl)ethoxy]methyl]-1,3-benzodiazole

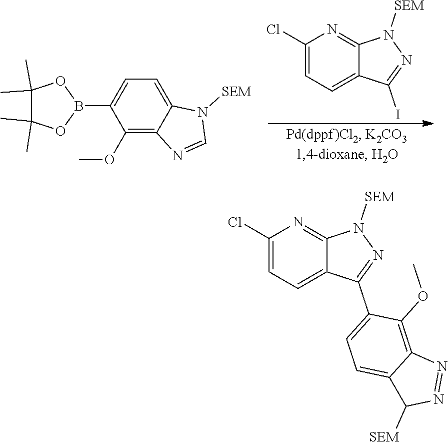 [SMILES: COc1c(B2OC(C)(C)C(C)(C)O2)ccc2c1ncn2COCC[Si](C)(C)C ; C[Si](C)(C)CCOCn1nc(I)c2ccc(Cl)nc21 ; COc1c(-c2nn(COCC[Si](C)(C)C)c3nc(Cl)ccc23)ccc2c1N=NC2COCC[Si](C)(C)C]

To a solution of 7-methoxy-6-(4,4,5,5-tetramethyl-1,3,2-dioxaborolan-2-yl)-1-[[2-(trimethylsilyl)ethoxy]methyl]-1,3-benzodiazole (500.0 mg, 0.62 mmol) in 1,4-dioxane/H₂O (10.0 mL/2.0 mL) was added 6-chloro-3-iodo-1-[[2-(trimethylsilyl)ethoxy]methyl]pyrazolo[3,4-b]pyridine (211.1 mg, 0.52 mmol), K₂CO₃ (213.6 mg, 1.55 mmol) and Pd(dppf)Cl₂ (37.7 mg, 0.05 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (9/1, v/v) to afford 5-(6-chloro-1-[[2-(trimethylsilyl)ethoxy]methyl]pyrazolo[3,4-b]pyridin-3-yl)-4-methoxy-1-[[2-(trimethylsilyl)ethoxy]methyl]-1,3-benzodiazole (200.0 mg, 69%) as a yellow oil. LCMS (ESI, m/z): [M+H]⁺=560.2.

### Step 2: Synthesis of (1S,2S)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrazolo[3,4-b]pyridin-6-yl]cyclopropane-1-carboxamide

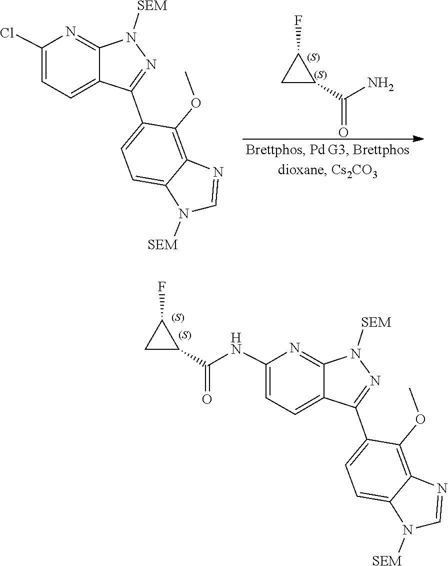

To a solution of 5-(6-chloro-1-[[2-(trimethylsilyl)ethoxy]methyl]pyrazolo[3,4-b]pyridin-3-yl)-4-methoxy-1-[[2-(trimethylsilyl)ethoxy]methyl]-1,3-benzodiazole (150.0 mg, 0.27 mmol) in 1,4-dioxane (10.0 mL) was added (1S,2S)-2-fluorocyclopropane-1-carboxamide (138.0 mg, 1.34 mmol), BrettPhos (28.7 mg, 0.05 mmol), Cs₂CO₃ (261.7 mg, 0.80 mmol) and BrettPhos Pd G3 (24.3 mg, 0.03 mmol) at room temperature under N₂. The resulting mixture was stirred at 100° C. for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/12, v/v) to afford (1S,2S)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrazolo[3,4-b]pyridin-6-yl]cyclopropane-1-carboxamide (79.0 mg, 47%) as a yellow oil. LCMS (ESI, m/z): [M+H]⁺=627.3.

### Step 3: Synthesis of (1S,2S)-2-fluoro-N-[3-(4-methoxy-1H-1,3-benzodiazol-5-yl)-1H-pyrazolo[3,4-b]pyridin-6-yl]cyclopropane-1-carboxamide (Compound 90)

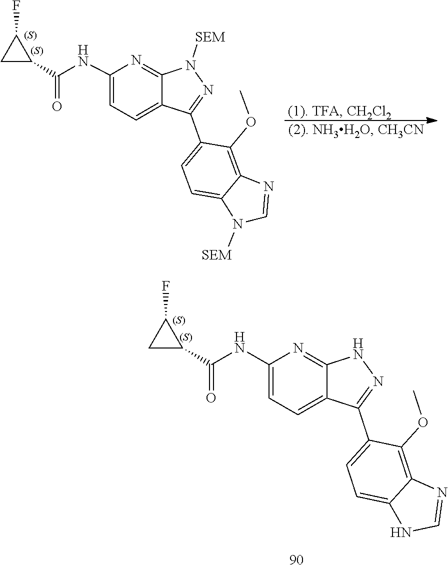

To a solution of (1S,2S)-2-fluoro-N-[3-(4-methoxy-1-[[2-(trimethylsilyl)ethoxy]methyl]-1,3-benzodiazol-5-yl)-1-[[2-(trimethylsilyl)ethoxy]methyl]pyrazolo[3,4-b]pyridin-6-yl]cyclopropane-1-carboxamide (110.0 mg, 0.18 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in CH₃CN (5.0 mL) was added NH₃·H₂O (5.0 mL) at room temperature. The resulting mixture was stirred at room temperature for another 2 h. After the reaction was completed, the resulting mixture was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 11% B to 32% B in 7 min; 254 nm) to afford (1S,2S)-2-fluoro-N-[3-(4-methoxy-1H-1,3-benzodiazol-5-yl)-1H-pyrazolo[3,4-b]pyridin-6-yl]cyclopropane-1-carboxamide (8.8 mg, 14%) as a white solid. LCMS (ESI, m/z): [M+H]⁺=367.2. ¹H NMR (400 MHz, DMSO-d₆): δ 13.36 (s, 1H), 12.61 (s, 1H), 10.96 (s, 1H), 8.28-8.23 (m, 1H), 8.15 (d, J=8.8 Hz, 1H), 7.99-7.96 (m, 1H), 7.50-7.44 (m, 1H), 7.27 (d, J=8.4 Hz, 1H), 5.04-4.85 (m, 1H), 4.29 (s, 3H), 2.33-2.23 (m, 1H), 1.73-1.62 (m, 1H), 1.23-1.15 (m, 1H).

### Example S91. Compound 91

### Step 1: Synthesis of (1S,2R)-2-(hydroxymethyl)-N-[3-(2-methoxyphenyl)-1H-pyrrolo[2,3-b]pyridin-6-yl]cyclopropane-1-carboxamide (Compound 91)

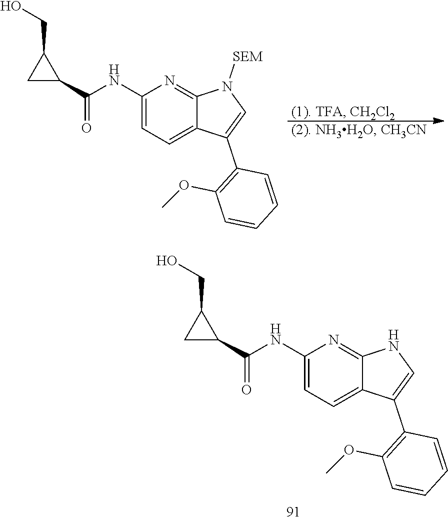 [SMILES: COc1ccccc1-c1cn(COCC[Si](C)(C)C)c2nc(NC(=O)[C@H]3C[C@H]3CO)ccc12 ; COc1ccccc1-c1c[nH]c2nc(NC(=O)[C@H]3C[C@H]3CO)ccc12]

To a solution of (1S,2R)-2-(hydroxymethyl)-N-[3-(2-methoxyphenyl)-1-[[2-(trimethylsilyl)ethoxy]methyl]pyrrolo[2,3-b]pyridin-6-yl]cyclopropane-1-carboxamide (34.0 mg, 0.07 mmol) in CH₂Cl₂ (2.0 mL) was added TFA (2.0 mL) at room temperature. The resulting mixture was stirred at room temperature for 3 h. The resulting mixture was concentrated under reduced pressure. The residue was re-dissolved in CH₃CN (2.0 mL) and NH₃·H₂O (2.0 mL). The resulting mixture was stirred at room temperature for 16 h. After the reaction was completed, the resulting mixture was concentrated under reduced pressure, then the was purified by Prep-HPLC with the following conditions (Column: XBridge Prep OBD C18 Column, 19×250 mm, 5 um; Mobile Phase A: Water (10 mmol/L NH₄HCO₃), Mobile Phase B: ACN; Flow rate: 25 mL/min; Gradient: 31% B to 40% B in 10 min; 254 nm) to afford (1S,2R)-2-(hydroxymethyl)-N-[3-(2-methoxyphenyl)-1H-pyrrolo[2,3-b]pyridin-6-yl]cyclopropane-1-carboxamide (2.7 mg, 11%) as a white solid. LCMS (ESI, m/z): [M+H]⁺=338.2. ¹H NMR (400 MHz, DMSO-d₆): δ 11.50 (s, 1H), 10.44 (s, 1H), 7.96-7.87 (m, 2H), 7.54-7.51 (m, 2H), 7.28-7.24 (m, 1H), 7.10 (d, J=8.0 Hz, 1H), 7.03-6.99 (m, 1H), 4.45-4.42 (m, 1H), 3.80 (s, 3H), 3.67-3.61 (m, 1H), 3.55-3.49 (m, 1H), 2.12-2.07 (m, 1H), 1.45-1.39 (m, 1H), 0.99-0.85 (m, 2H).

### Example S92. Compound 92

### Step 1: Synthesis of 3-(6-chloro-1-[[2-(trimethylsilyl)ethoxy]methyl]pyrrolo[2,3-b]pyridin-3-yl)-5-fluoro-2-methoxypyridine

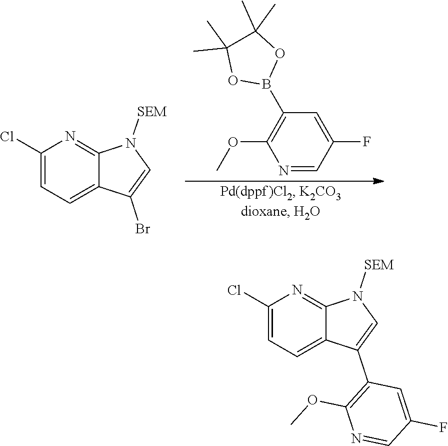 [SMILES: C[Si](C)(C)CCOCn1cc(Br)c2ccc(Cl)nc21 ; COc1ncc(F)cc1B1OC(C)(C)C(C)(C)O1 ; COc1ncc(F)cc1-c1cn(COCC[Si](C)(C)C)c2nc(Cl)ccc12]

To a mixture of 5-fluoro-2-methoxy-3-(4,4,5,5-tetramethyl-1,3,2-dioxaborolan-2-yl)pyridine (300.0 mg, 1.75 mmol) and 3-bromo-6-chloro-1-[[2-(trimethylsilyl)ethoxy]methyl]pyrrolo[2,3-b]pyridine (634.8 mg, 1.75 mmol) in dioxane/H₂O (10.0 mL/1.0 mL) was added Pd(dppf)Cl₂ (128.4 mg, 0.17 mmol) and K₂CO₃ (727.7 mg, 5.26 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (20/1, v/v) to afford 3-(6-chloro-1-[[2-(trimethylsilyl)ethoxy]methyl]pyrrolo[2,3-b]pyridin-3-yl)-5-fluoro-2-methoxypyridine (380.0 mg, 53%) as a yellow oil. LCMS (ESI, m/z): [M+H]⁺=408.1

### Step 2: Synthesis of (1S,2S)-2-fluoro-N-[3-(5-fluoro-2-methoxypyridin-3-yl)-1-[[2-(trimethylsilyl)ethoxy]methyl]pyrrolo[2,3-b]pyridin-6-yl]cyclopropane-1-carboxamide

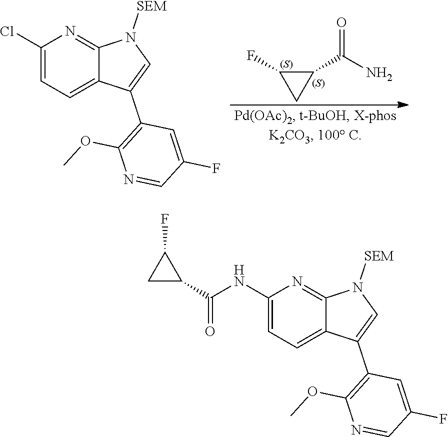

To a mixture of 3-(6-chloro-1-[[2-(trimethylsilyl)ethoxy]methyl]pyrrolo[2,3-b]pyridin-3-yl)-5-fluoro-2-methoxypyridine (280.0 mg, 0.68 mmol) and (1S,2S)-2-fluorocyclopropane-1-carboxamide (353.8 mg, 3.43 mmol) in BuOH (5.0 mL) was added Pd(OAc)₂ (30.8 mg, 0.13 mmol), K₂CO₃ (284.5 mg, 2.05 mmol) and X-Phos (65.4 mg, 0.13 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) afford (1S,2S)-2-fluoro-N-[3-(5-fluoro-2-methoxypyridin-3-yl)-1-[[2-(trimethylsilyl)ethoxy]methyl]pyrrolo[2,3-b]pyridin-6-yl]cyclopropane-1-carboxamide (170.0 mg, 52%) as a yellow oil. LCMS (ESI, m/z): [M+H]⁺=475.2

### Step 3: Synthesis of (1S,2S)-2-fluoro-N-[3-(5-fluoro-2-methoxypyridin-3-yl)-1H-pyrrolo[2,3-b]pyridin-6-yl]cyclopropane-1-carboxamide (Compound 92)

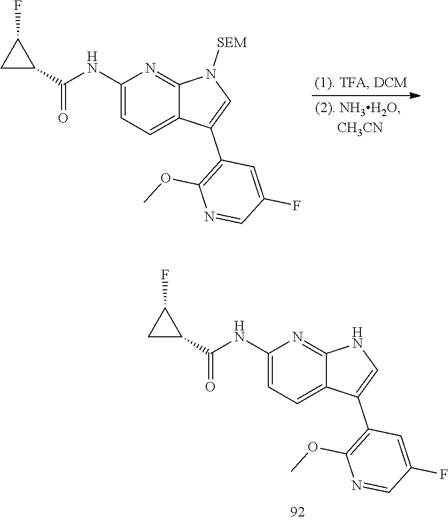 [SMILES: COc1ncc(F)cc1-c1cn(COCC[Si](C)(C)C)c2nc(NC(=O)[C@@H]3C[C@@H]3F)ccc12 ; COc1ncc(F)cc1-c1c[nH]c2nc(NC(=O)[C@@H]3C[C@@H]3F)ccc12]

To a solution of (1S,2S)-2-fluoro-N-[3-(5-fluoro-2-methoxypyridin-3-yl)-1-[[2-(trimethylsilyl)ethoxy]methyl]pyrrolo[2,3-b]pyridin-6-yl]cyclopropane-1-carboxamide (170.0 mg, 0.35 mmol) in DCM (3.0 mL) was added TFA (2.0 mL) at room temperature. The resulting mixture was stirred at room temperature for 2 h. The resulting mixture was concentrated under reduced pressure. To the above residue was added NH₃·H₂O (2.0 mL) and ACN (1.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18 30×250 mm, 5 um; Mobile Phase A: Water (10 mmol/L NH₄HCO₃), Mobile Phase B: ACN; Flow rate: 60 mL/min; Gradient: 41% B to 50% B in 8 min; 254 nm) to afford (1S,2S)-2-fluoro-N-[3-(5-fluoro-2-methoxypyridin-3-yl)-1H-pyrrolo[2,3-b]pyridin-6-yl]cyclopropane-1-carboxamide (37.0 mg, 30%) as a white solid. LCMS (ESI, m/z): [M+H]⁺=345.1. ¹H NMR (300 MHz, DMSO-d₆): δ 11.83 (s, 1H), 10.66 (s, 1H), 8.18 (d, J=8.7 Hz, 1H), 8.04 (d, J=3.0 Hz, 1H), 7.96-7.90 (m, 2H), 7.83 (d, J=2.7 Hz, 1H), 5.06-4.79 (m, 1H), 3.95 (s, 3H), 2.26-2.21 (m, 1H), 1.73-1.59 (m, 1H), 1.21-1.12 (m, 1H).

### Example S93. Compound 93

### Step 1: Synthesis of tert-butyl 5-bromo-6-methoxy-1H-pyrrolo[2,3-b]pyridine-1-carboxylate

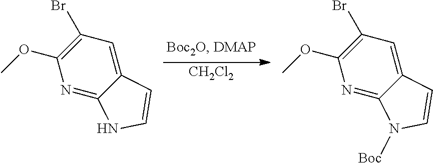

To a solution of 5-bromo-6-methoxy-1H-pyrrolo[2,3-b]pyridine (300.0 mg, 1.32 mmol) in CH₂Cl₂ (10.0 mL) was added Boc₂O (285.8 mg, 1.32 mmol) and DMAP (160.0 mg, 1.32 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (92/8, v/v) to afford tert-butyl 5-bromo-6-methoxy-1H-pyrrolo[2,3-b]pyridine-1-carboxylate (350.0 mg, 81%) as a white solid. LCMS (ESI, m/z): [M+H]⁺=327.0.

### Step 2: Synthesis of tert-butyl 6-chloro-6′-methoxy-1-((2-(trimethylsilyl)ethoxy)methyl)-1H,1′H-[3,5′-bipyrrolo[2,3-b]pyridine]-1′-carboxylate

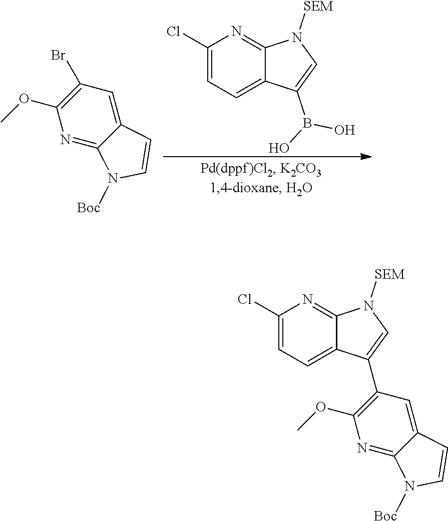 [SMILES: COc1nc2c(ccn2C(=O)OC(C)(C)C)cc1Br ; C[Si](C)(C)CCOCn1cc(B(O)O)c2ccc(Cl)nc21 ; COc1nc2c(ccn2C(=O)OC(C)(C)C)cc1-c1cn(COCC[Si](C)(C)C)c2nc(Cl)ccc12]

To a solution of tert-butyl 5-bromo-6-methoxy-1H-pyrrolo[2,3-b]pyridine-1-carboxylate (320.0 mg, 0.98 mmol) in 1,4-dioxane/H₂O (10.0/2.0 mL) was added (6-chloro-1-((2-(trimethylsilyl)ethoxy)methyl)-1H-pyrrolo[2,3-b]pyridin-3-yl)boronic acid (319.5 mg, 0.98 mmol), K₂CO₃ (405.5 mg, 2.93 mmol) and Pd(dppf)Cl₂ (71.5 mg, 0.10 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (94/6, v/v) to afford tert-butyl 6-chloro-6′-methoxy-1-((2-(trimethylsilyl)ethoxy)methyl)-1H,1′H-[3,5′-bipyrrolo[2,3-b]pyridine]-1′-carboxylate (310.0 mg, 59%) as a colorless oil. LCMS (ESI, m/z): [M+H]⁺=529.2.

### Step 3: Synthesis of tert-butyl 6-((1S,2S)-2-fluorocyclopropane-1-carboxamido)-6′-methoxy-1-((2-(trimethylsilyl)ethoxy)methyl)-1H,1′H-[3,5′-bipyrrolo[2,3-b]pyridine]-1′-carboxylate

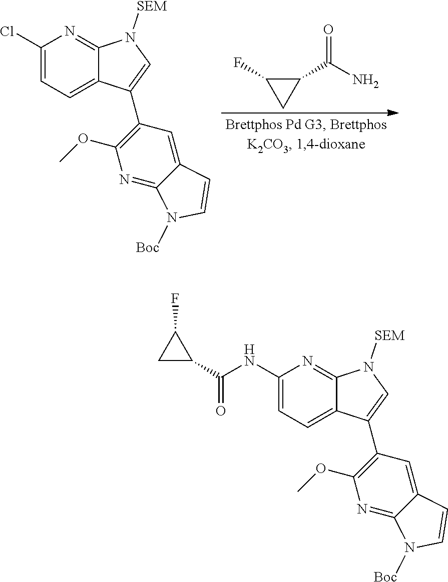 [SMILES: COc1nc2c(ccn2C(=O)OC(C)(C)C)cc1-c1cn(COCC[Si](C)(C)C)c2nc(Cl)ccc12 ; NC(=O)[C@@H]1C[C@@H]1F ; COc1nc2c(ccn2C(=O)OC(C)(C)C)cc1-c1cn(COCC[Si](C)(C)C)c2nc(NC(=O)[C@@H]3C[C@@H]3F)ccc12]

To a solution of tert-butyl 6-chloro-6′-methoxy-1-((2-(trimethylsilyl)ethoxy)methyl)-1H,1′H-[3,5′-bipyrrolo[2,3-b]pyridine]-1′-carboxylate (160.0 mg, 0.30 mmol) in 1,4-dioxane (6.0 mL) was added (1S,2S)-2-fluorocyclopropane-1-carboxamide (155.9 mg, 1.51 mmol), K₂CO₃ (125.4 mg, 0.91 mmol), BrettPhos (32.5 mg, 0.06 mmol) and BrettPhos Pd G3 (27.4 mg, 0.03 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (78/22, v/v) to afford tert-butyl 6-((1S,2S)-2-fluorocyclopropane-1-carboxamido)-6′-methoxy-1-((2-(trimethylsilyl)ethoxy)methyl)-1H,1′H-[3,5′-bipyrrolo[2,3-b]pyridine]-1′-carboxylate (180.0 mg, 99%) as a yellow solid. LCMS (ESI, m/z): [M+H]⁺=596.3.

### Step 4: Synthesis of (1S,2S)-2-fluoro-N-(6′-methoxy-1H,1′H-[3,5′-bipyrrolo[2,3-b]pyridin]-6-yl)cyclopropane-1-carboxamide (Compound 93)

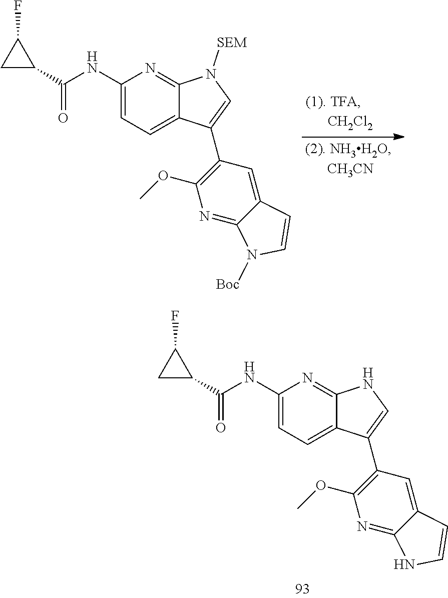 [SMILES: COc1nc2c(ccn2C(=O)OC(C)(C)C)cc1-c1cn(COCC[Si](C)(C)C)c2nc(NC(=O)[C@@H]3C[C@@H]3F)ccc12 ; COc1nc2[nH]ccc2cc1-c1c[nH]c2nc(NC(=O)[C@@H]3C[C@@H]3F)ccc12]

To a solution of tert-butyl 6-((1S,2S)-2-fluorocyclopropane-1-carboxamido)-6′-methoxy-1-((2-(trimethylsilyl)ethoxy)methyl)-1H,1′H-[3,5′-bipyrrolo[2,3-b]pyridine]-1′-carboxylate (160.0 mg, 0.27 mmol) in CH₂Cl₂ (5.0 mL) was added TFA (5.0 mL) at room temperature. The resulting mixture was stirred at room temperature for 4 h. After the reaction was completed, the resulting mixture was concentrated under reduced pressure. The residue was dissolved in CH₃CN (5.0 mL) and NH₃·H₂O (5.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xcelect CSH F-pheny OBD Column, 19×250 mm, 5 um; Mobile Phase A: Water (10 mmol/L NH₄HCO₃), Mobile Phase B: MeOH—Preparative; Flow rate: 25 mL/min; Gradient: 46% B to 61% B in 9 min; 254 nm) to afford (1S,2S)-2-fluoro-N-(6′-methoxy-1H,1′H-[3,5′-bipyrrolo[2,3-b]pyridin]-6-yl)cyclopropane-1-carboxamide (18.8 mg, 19%) as a white solid. LCMS (ESI, m/z): [M+H]⁺=366.2. ¹H NMR (400 MHz, DMSO-d₆): δ 11.52-11.46 (m, 2H), 10.62 (s, 1H), 8.05-8.01 (m, 2H), 7.89 (d, J=8.4 Hz, 1H), 7.56 (d, J=2.4 Hz, 1H), 7.21-7.20 (m, 1H), 6.40-6.39 (m, 1H), 5.03-4.82 (m, 1H), 3.94 (s, 3H), 2.24-2.22 (m, 1H), 1.71-1.61 (m, 1H), 1.18-1.16 (m, 1H).

### Example S94. Compound 94

### Step 1: Synthesis of 6-chloro-3-(2-cyclopropoxyphenyl)-1-((2-(trimethylsilyl)ethoxy)methyl)-1H-pyrazolo[3,4-b]pyridine

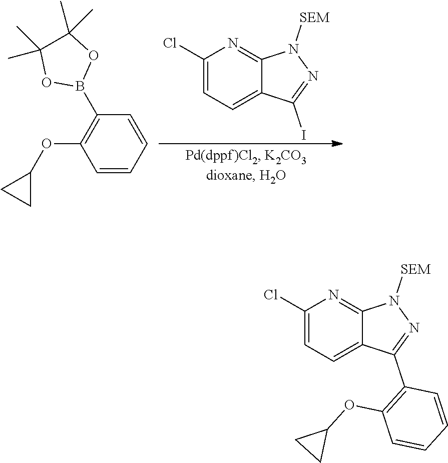 [SMILES: CC1(C)OB(c2ccccc2OC2CC2)OC1(C)C ; C[Si](C)(C)CCOCn1nc(I)c2ccc(Cl)nc21 ; C[Si](C)(C)CCOCn1nc(-c2ccccc2OC2CC2)c2ccc(Cl)nc21]

To a solution of 2-(2-cyclopropoxyphenyl)-4,4,5,5-tetramethyl-1,3,2-dioxaborolane (320.0 mg, 1.23 mmol) in dioxane/H₂O (16.0/4.0 mL) was added 6-chloro-3-iodo-1-((2-(trimethylsilyl)ethoxy)methyl)-1H-pyrazolo[3,4-b]pyridine (504.0 mg, 1.23 mmol), K₂CO₃ (510.0 mg, 3.69 mmol) and Pd(dppf)Cl₂ (90.0 mg, 0.12 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2-cyclopropoxyphenyl)-1-((2-(trimethylsilyl)ethoxy)methyl)-1H-pyrazolo[3,4-b]pyridine (300.0 mg, 58%) as a yellow solid. LCMS (ESI, m/z): [M+H]⁺=416.1.

### Step 2: Synthesis of (1S,2S)—N-(3-(2-cyclopropoxyphenyl)-1-((2-(trimethylsilyl)ethoxy)methyl)-1H-pyrazolo[3,4-b]pyridin-6-yl)-2-fluorocyclopropane-1-carboxamide

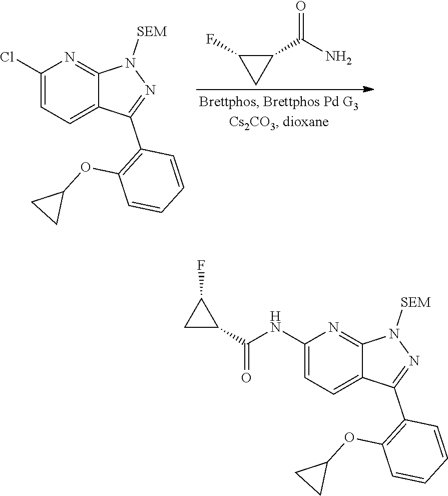 [SMILES: C[Si](C)(C)CCOCn1nc(-c2ccccc2OC2CC2)c2ccc(Cl)nc21 ; NC(=O)[C@@H]1C[C@@H]1F ; C[Si](C)(C)CCOCn1nc(-c2ccccc2OC2CC2)c2ccc(NC(=O)[C@@H]3C[C@@H]3F)nc21]

To a solution of 6-chloro-3-(2-cyclopropoxyphenyl)-1-((2-(trimethylsilyl)ethoxy)methyl)-1H-pyrazolo[3,4-b]pyridine (260.0 mg, 0.63 mmol) in dioxane (10.0 mL) was added (1S,2S)-2-fluorocyclopropane-1-carboxamide (322.2 mg, 3.13 mmol), Cs₂CO₃ (610.9 mg, 1.88 mmol), Brettphos (67.1 mg, 0.13 mmol) and BrettPhos Pd G3 (56.7 mg, 0.06 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford (1S,2S)—N-(3-(2-cyclopropoxyphenyl)-1-((2-(trimethylsilyl)ethoxy)methyl)-1H-pyrazolo[3,4-b]pyridin-6-yl)-2-fluorocyclopropane-1-carboxamide (110.0 mg, 36%) as a yellow solid. LCMS (ESI, m/z): [M+H]⁺=483.2.

### Step 3: Synthesis of (1S,2S)—N-(3-(2-cyclopropoxyphenyl)-1H-pyrazolo[3,4-b]pyridin-6-yl)-2-fluorocyclopropane-1-carboxamide (Compound 94)

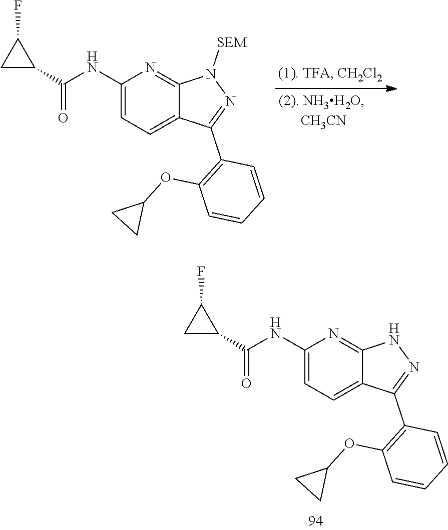 [SMILES: C[Si](C)(C)CCOCn1nc(-c2ccccc2OC2CC2)c2ccc(NC(=O)[C@@H]3C[C@@H]3F)nc21 ; O=C(Nc1ccc2c(-c3ccccc3OC3CC3)n[nH]c2n1)[C@@H]1C[C@@H]1F]

To a solution of (1S,2S)—N-(3-(2-cyclopropoxyphenyl)-1-((2-(trimethylsilyl)ethoxy)methyl)-1H-pyrazolo[3,4-b]pyridin-6-yl)-2-fluorocyclopropane-1-carboxamide (90.0 mg, 0.18 mmol) in CH₂Cl₂ (5.0 mL) was added TFA (5.0 mL) at room temperature. The resulting mixture was stirred at room temperature for 3 h. The resulting mixture was concentrated under reduced pressure. The residue was re-dissolved in CH₃CN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XSelect CSH Prep C18 OBD Column 19×250 mm, 5 um; Mobile Phase A: Water (0.1% FA), Mobile Phase B: MeOH; Flow rate: 25 mL/min; Gradient: 57% to 69% in 12 min; 254 nm) to afford (1S,2S)—N-(3-(2-cyclopropoxyphenyl)-1H-pyrazolo[3,4-b]pyridin-6-yl)-2-fluorocyclopropane-1-carboxamide (14.0 mg, 21%) as a white solid. LCMS (ESI, m/z): [M+H]⁺=353.2. ¹H NMR (300 MHz, DMSO-d₆): δ 13.48 (s, 1H), 10.98 (s, 1H), 8.06-7.96 (m, 2H), 7.63-7.60 (m, 1H), 7.50-7.46 (m, 2H), 7.12-7.07 (m, 1H), 5.08-4.84 (m, 1H), 3.93-3.89 (m, 1H), 2.29-2.25 (m, 1H), 1.72-1.63 (m, 1H), 1.23-1.16 (m, 1H), 0.83-0.66 (m, 4H).

### Example S95. Compound 95

### Step 1: Synthesis of 6-bromo-3,4-difluoro-2-nitrophenol

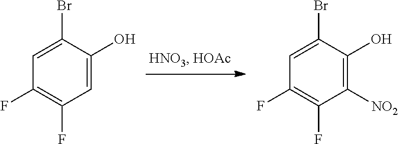 [SMILES: Oc1cc(F)c(F)cc1Br ; O=[N+]([O-])c1c(O)c(Br)cc(F)c1F]

To a solution of 2-bromo-4,5-difluorophenol (3.7 g, 17.94 mmol) in HOAc (20.0 mL) was added HNO₃ (1.4 g, 21.5 mmol). The mixture was stirred at room temperature for 2 h. After the reaction was completed, the reaction mixture was quenched with water and extracted with ethyl acetate. The combined organic layer was dried over anhydrous sodium sulfate and filtered. The filtrate was concentrated under vacuum to afford 6-bromo-3,4-difluoro-2-nitrophenol (3.0 g, crude) as a yellow solid. LCMS (ESI, m/z): [M+H]⁺=253.9.

### Step 2: Synthesis of 1-bromo-4,5-difluoro-2-methoxy-3-nitrobenzene

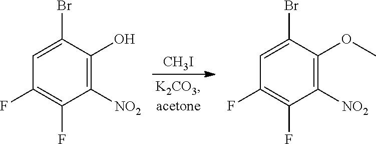

To a mixture of 6-bromo-3,4-difluoro-2-nitrophenol (2.0 g, 7.87 mmol) and K₂CO₃ (2.2 g, 15.75 mmol) in acetone (40.0 mL) was added methyl iodide (2.2 g, 15.75 mmol). The mixture was stirred at 70° C. for 16 h. After the reaction was completed, the resulting mixture was filtered. The filtrate was concentrated under reduced pressure to afford 1-bromo-4,5-difluoro-2-methoxy-3-nitrobenzene (2.0 g, crude) as a yellow solid. LCMS (ESI, m/z): [M+H]⁺=267.9.

### Step 3: Synthesis of 4-bromo-6-fluoro-3-methoxy-2-nitroaniline

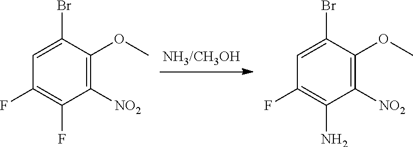

A mixture of 1-bromo-4,5-difluoro-2-methoxy-3-nitrobenzene (2.0 g, 7.46 mmol) in NH₃/MeOH (16.0 mL, 7 mol/L) was stirred at 60° C. for 2 h. After the reaction was completed, the mixture was concentrated under reduced pressure to afford 4-bromo-6-fluoro-3-methoxy-2-nitroaniline (1.8 g, crude) as a yellow solid. LCMS (ESI, m/z): [M+H]⁺=265.0.

### Step 4: Synthesis of 4-bromo-6-fluoro-3-methoxybenzene-1,2-diamine

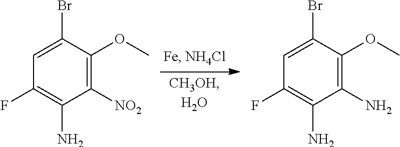

To a mixture of 4-bromo-6-fluoro-3-methoxy-2-nitroaniline (1.0 g, 3.77 mmol) and NH₄Cl (1.0 g, 18.86 mmol) in MeOH (50.0 mL) and H₂O (5.0 mL) was added Fe (2.1 g, 37.73 mmol). The mixture was stirred at 80° C. for 2 h. After the reaction was completed, the resulting mixture was filtered. The filtrate was concentrated under reduced pressure. The residue was purified by flash column chromatography with petroleum ether/ethyl acetate (50/50, v/v) to afford 4-bromo-6-fluoro-3-methoxybenzene-1,2-diamine (500.0 mg, 56%) as a brown oil. LCMS (ESI, m/z): [M+H]⁺=235.0.

### Step 5: Synthesis of 5-bromo-7-fluoro-4-methoxy-1H-1,3-benzodiazole

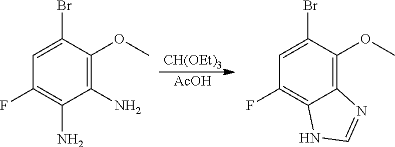

A mixture of 4-bromo-6-fluoro-3-methoxybenzene-1,2-diamine (400.0 mg, 8.51 mmol) and triethyl orthoformate (5.0 mL) in acetic Acid (5.0 mL) was stirred at 100° C. for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum to afford 5-bromo-7-fluoro-4-methoxy-1H-1,3-benzodiazole (400.0 mg, crude) as a yellow solid. LCMS (ESI, m/z): [M+H]⁺=245.0.

### Step 6: Synthesis of 5-bromo-7-fluoro-4-methoxy-1-[[2-(trimethylsilyl)ethoxy]methyl]-1,3-benzodiazole

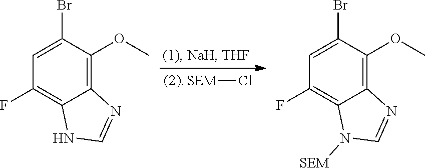

To a solution of 5-bromo-7-fluoro-4-methoxy-1H-1,3-benzodiazole (510.0 mg, 2.08 mmol) in THE (5.0 mL) was added sodium hydride (100.0 mg, 60%) at 0° C. The mixture was stirred at 0° C. for 15 min. Then SEM-Cl (381.8 mg, 2.29 mmol) was added to the mixture. The mixture was stirred at room temperature for another 2 h. After the reaction was completed, the reaction mixture was quenched with water and extracted with DCM. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford 5-bromo-7-fluoro-4-methoxy-1-[[2-(trimethylsilyl)ethoxy]methyl]-1,3-benzodiazole (510.0 mg, 65%) as a colorless oil. LCMS (ESI, m/z): [M+H]⁺=375.0.

### Step 7: Synthesis of 5-(6-chloro-1-[[2-(trimethylsilyl)ethoxy]methyl]pyrrolo[2,3-b]pyridin-3-yl)-7-fluoro-4-methoxy-1-[[2-(trimethylsilyl)ethoxy]methyl]-1,3-benzodiazole

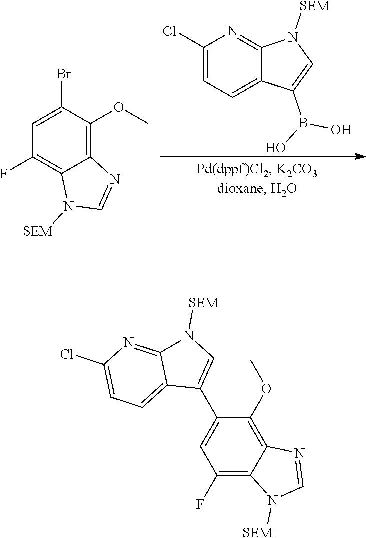 [SMILES: COc1c(Br)cc(F)c2c1ncn2COCC[Si](C)(C)C ; C[Si](C)(C)CCOCn1cc(B(O)O)c2ccc(Cl)nc21 ; COc1c(-c2cn(COCC[Si](C)(C)C)c3nc(Cl)ccc23)cc(F)c2c1ncn2COCC[Si](C)(C)C]

To a solution of 5-bromo-7-fluoro-4-methoxy-1-[[2-(trimethylsilyl)ethoxy]methyl]-1,3-benzodiazole (400.0 mg, 1.06 mmol) in dioxane/H₂O (10.0 mL/1.0 mL) was added 6-chloro-1-[[2-(trimethylsilyl)ethoxy]methyl]pyrrolo[2,3-b]pyridin-3-ylboronic acid (382.9 mg, 1.17 mmol), K₂CO₃ (441.9 mg, 3.20 mmol) and Pd(dppf)Cl₂ (77.9 mg, 0.11 mmol). The mixture was stirred at 80° C. for 16 h under N₂. After the reaction was completed, the resulting mixture was diluted with water and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford 5-(6-chloro-1-[[2-(trimethylsilyl)ethoxy]methyl]pyrrolo[2,3-b]pyridin-3-yl)-7-fluoro-4-methoxy-1-[[2-(trimethylsilyl)ethoxy]methyl]-1,3-benzodiazole (330.0 mg, 54%) as a brown oil. LCMS (ESI, m/z): [M+H]⁺=577.2.

### Step 8: Synthesis of (1S,2S)-2-fluoro-N-(3-(7-fluoro-4-methoxy-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide

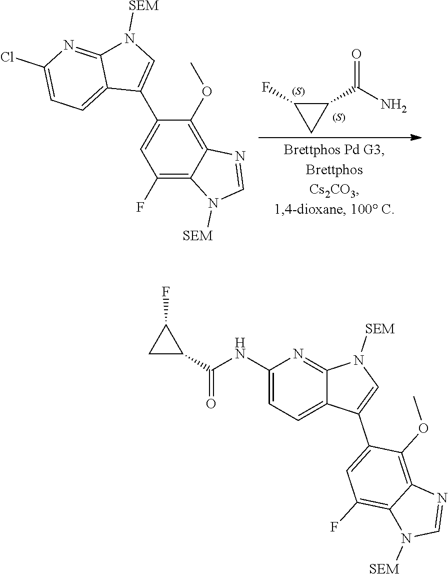

To a stirred mixture of 5-(6-chloro-1-[[2-(trimethylsilyl)ethoxy]methyl]pyrrolo[2,3-b]pyridin-3-yl)-7-fluoro-4-methoxy-1-[[2-(trimethylsilyl)ethoxy]methyl]-1,3-benzodiazole (250.0 mg, 0.43 mmol) and (1S,2S)-2-fluorocyclopropane-1-carboxamide (89.3 mg, 0.86 mmol) in 1,4-dioxane (5.0 mL) was added BrettPhos (92.9 mg, 0.17 mmol), BrettPhos Pd G3 (78.5 mg, 0.08 mmol) and Cs₂CO₃ (423.3 mg, 1.29 mmol). The resulting mixture was stirred at 100° C. for 16 h under N₂. After the reaction was completed, the resulting mixture was diluted with water and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) to afford (1S,2S)-2-fluoro-N-(3-(7-fluoro-4-methoxy-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide (100.0 mg, 45%) as a brown solid. LCMS (ESI, m/z): [M+H]⁺=644.3.

### Step 9: Synthesis of (1S,2S)-2-fluoro-N-(3-(7-fluoro-4-methoxy-1H-benzo[d]imidazol-5-yl)-1H-pyrrolo[2,3-b]pyridin-6-yl)cyclopropanecarboxamide (Compound 95)

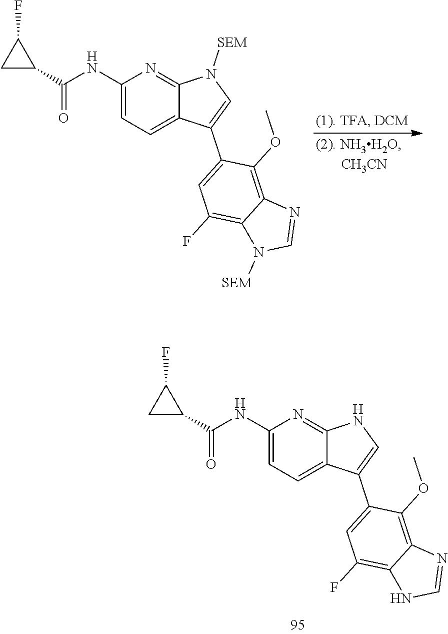 [SMILES: COc1c(-c2cn(COCC[Si](C)(C)C)c3nc(NC(=O)[C@@H]4C[C@@H]4F)ccc23)cc(F)c2c1ncn2COCC[Si](C)(C)C ; COc1c(-c2c[nH]c3nc(NC(=O)[C@@H]4C[C@@H]4F)ccc23)cc(F)c2[nH]cnc12]

A mixture of (1S,2S)-2-fluoro-N-(3-(7-fluoro-4-methoxy-1-((2-(trimethylsilyl)ethoxy)methyl)-1H-benzo[d]imidazol-5-yl)-1-((2-(trimethylsilyl)ethoxy)methyl)-1H-pyrrolo[2,3-b]pyridin-6-yl)cyclopropanecarboxamide (100.0 mg, 0.19 mmol) and TFA (2.0 mL) in DCM (2.0 mL) was stirred at room temperature for 2 h. The resulting mixture was concentrated under reduced pressure. To the above residue was added ACN (2.0 mL) and NH₃·H₂O (2.0 mL). The resulting mixture was stirred at room temperature for another 4 h. After the reaction was completed, the resulting mixture was concentrated under vacuum. The residue was purified by Prep-HPLC with the following conditions: Column: Xselect CSH OBD Column 30×150 mm, 5 um; Mobile Phase A: Water, Mobile Phase B: ACN; Flow rate: 60 mL/min; Gradient: 5% B to 80% B in 7 min; 254/220 nm to afford (1S,2S)-2-fluoro-N-(3-(7-fluoro-4-methoxy-1H-benzo[d]imidazol-5-yl)-1H-pyrrolo[2,3-b]pyridin-6-yl)cyclopropanecarboxamide (3.9 mg, 5%) as a white solid. LCMS (ESI, m/z): [M+H]⁺=384.1. ¹H NMR (300 MHz, DMSO-d₆): δ 13.09 (s, 1H), 11.67 (s, 1H), 10.64 (s, 1H), 8.25 (s, 1H), 8.09-8.02 (m, 1H), 7.90 (d, J=8.4 Hz, 1H), 7.69-7.46 (m, 1H), 7.16-7.13 (m, 1H), 5.05-4.77 (m, 1H), 4.09 (s, 1H), 3.58 (s, 2H), 2.29-2.18 (m, 1H), 1.71-1.57 (m, 1H), 1.22-1.08 (m, 1H).

### Example S96. Compound 96

### Step 1: Synthesis of 5-bromo-6-methoxy-1-[[2-(trimethylsilyl)ethoxy]methyl]-1,2,3-benzotriazole

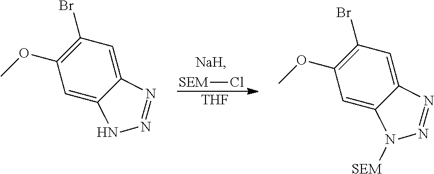

To a solution of 5-bromo-6-methoxy-1H-1,2,3-benzotriazole (200.0 mg, 0.87 mmol) in THE (10.0 mmL) was added NaH (52.6 mg, 60%) at 0° C. The mixture was stirred at 0° C. for 15 min. Then SEM-Cl (160.8 mg, 0.96 mmol) was added to the mixture. The mixture was stirred at room temperature for another 2 h. After the reaction was completed, the reaction mixture was quenched with water and extracted with DCM. The organic layer was washed with brine, dried over anhydrous sodium sulfate and filtered. The filtrate was concentrated under vacuum. The residue was purified by flash column chromatography with petroleum ether/ethyl acetate (75/25, v/v) to afford 5-bromo-6-methoxy-1-[[2-(trimethylsilyl)ethoxy]methyl]-1,2,3-benzotriazole (220.0 mg, 70%) as a yellow solid. LCMS (ESI, m/z): [M+H]⁺=358.1.

### Step 2: Synthesis of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,2,3-benzotriazole

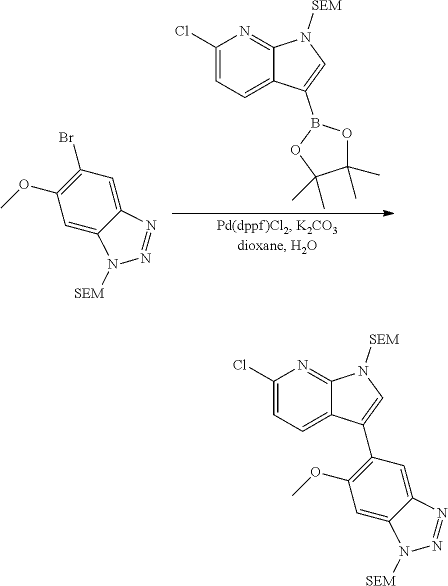 [SMILES: COc1cc2c(cc1Br)nnn2COCC[Si](C)(C)C ; CC1(C)OB(c2cn(COCC[Si](C)(C)C)c3nc(Cl)ccc23)OC1(C)C ; COc1cc2c(cc1-c1cn(COCC[Si](C)(C)C)c3nc(Cl)ccc13)nnn2COCC[Si](C)(C)C]

To a mixture of 5-bromo-6-methoxy-1-[[2-(trimethylsilyl)ethoxy]methyl]-1,2,3-benzotriazole (205.0 mg, 0.57 mmol) and 6-chloro-3-(4,4,5,5-tetramethyl-1,3,2-dioxaborolan-2-yl)-1-[[2-(trimethylsilyl)ethoxy]methyl]pyrrolo[2,3-b]pyridine (257.2 mg, 0.62 mmol) in dioxane (5.0 mL) and H₂O (0.50 mL) was added Pd(dppf)Cl₂ (83.7 mg, 0.11 mmol) and K₂CO₃ (237.2 mg, 1.71 mmol). The mixture was stirred at 80° C. for 16 h under N₂. After the reaction was completed, the reaction mixture was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,2,3-benzotriazole (250.0 mg, 78%) as a yellow oil. LCMS (ESI, m/z): [M+H]⁺=560.2.

### Step 3: Synthesis of (1S,2S)-2-fluoro-N-[3-(6-methoxy-1-[[2-(trimethylsilyl)ethoxy]methyl]-1,2,3-benzotriazol-5-yl)-1-[[2-(trimethylsilyl)ethoxy]methyl]pyrrolo[2,3-b]pyridin-6-yl]cyclopropane-1-carboxamide

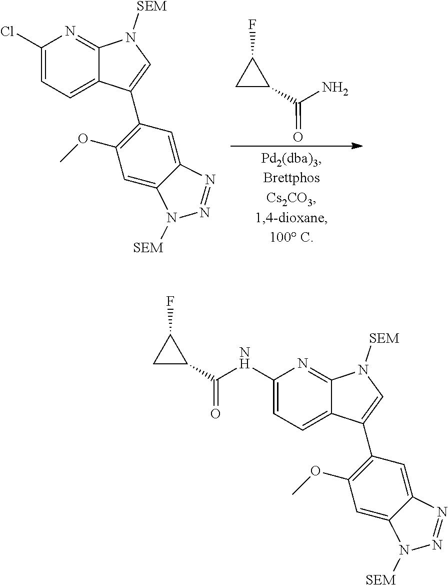 [SMILES: COc1cc2c(cc1-c1cn(COCC[Si](C)(C)C)c3nc(Cl)ccc13)nnn2COCC[Si](C)(C)C ; NC(=O)[C@@H]1C[C@@H]1F ; COc1cc2c(cc1-c1cn(COCC[Si](C)(C)C)c3nc(NC(=O)[C@@H]4C[C@@H]4F)ccc13)nnn2COCC[Si](C)(C)C]

To a mixture of 5-(6-chloro-1-[[2-(trimethylsilyl)ethoxy]methyl]pyrrolo[2,3-b]pyridin-3-yl)-6-methoxy-1-[[2-(trimethylsilyl)ethoxy]methyl]-1,2,3-benzotriazole (250.0 mg, 0.44 mmol) and (1S,2S)-2-fluorocyclopropane-1-carboxamide (230.0 mg, 2.23 mmol) in 1,4-dioxane (5.0 mL) was added BrettPhos (47.9 mg, 0.09 mmol), Pd₂(dba)₃ (163.4 mg, 0.18 mmol) and Cs₂CO₃ (436.1 mg, 1.33 mmol). The mixture was stirred at 100° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under vacuum. The residue was purified by reverse phase flash column chromatography with ACN/H₂O (30/70, v/v) to afford (1S,2S)-2-fluoro-N-[3-(6-methoxy-1-[[2-(trimethylsilyl)ethoxy]methyl]-1,2,3-benzotriazol-5-yl)-1-[[2-(trimethylsilyl)ethoxy]methyl]pyrrolo[2,3-b]pyridin-6-yl]cyclopropane-1-carboxamide (160.0 mg, 57%) as a yellow oil. LCMS (ESI, m/z): [M+H]⁺=627.3.

### Step 4: Synthesis of (1S,2S)-2-fluoro-N-[3-(6-methoxy-1H-1,2,3-benzotriazol-5-yl)-1H-pyrrolo[2,3-b]pyridin-6-yl]cyclopropane-1-carboxamide (Compound 96)

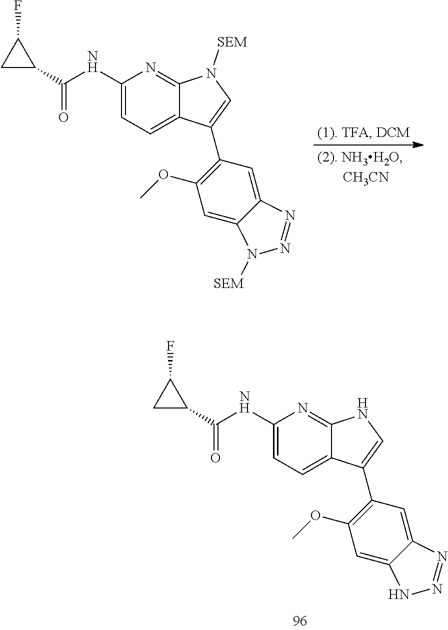 [SMILES: COc1cc2c(cc1-c1cn(COCC[Si](C)(C)C)c3nc(NC(=O)[C@@H]4C[C@@H]4F)ccc13)nnn2COCC[Si](C)(C)C ; COc1cc2[nH]nnc2cc1-c1c[nH]c2nc(NC(=O)[C@@H]3C[C@@H]3F)ccc12]

A mixture of (1S,2S)-2-fluoro-N-[3-(6-methoxy-1-[[2-(trimethylsilyl)ethoxy]methyl]-1,2,3-benzotriazol-5-yl)-1-[[2-(trimethylsilyl)ethoxy]methyl]pyrrolo[2,3-b]pyridin-6-yl]cyclopropane-1-carboxamide (150.0 mg, 0.24 mmol) and TFA (5.0 mL) in DCM (5.0 mL) was stirred at room temperature for 2 h. The resulting mixture was concentrated under reduced pressure. To the above residue was added ACN (5.0 mL) and NH₃·H₂O (5.0 mL). The resulting mixture was stirred at room temperature for another 4 h. After the reaction was completed, the resulting mixture was concentrated under vacuum. The residue was purified by Prep-HPLC with the following conditions: Column: Xselect CSH OBD Column 30×150 mm, 5 um; Mobile Phase A: Water, Mobile Phase B: ACN; Flow rate: 60 mL/min; Gradient: 5% B to 80% B in 7 min; 254/220 to afford (1S,2S)-2-fluoro-N-[3-(6-methoxy-1H-1,2,3-benzotriazol-5-yl)-1H-pyrrolo[2,3-b]pyridin-6-yl]cyclopropane-1-carboxamide (10.9 mg, 24%) as a white solid. LCMS (ESI, m/z): [M+H]⁺=367.1. ¹H NMR (300 MHz, DMSO-d₆): δ 15.33 (s, 1H), 11.63 (d, J=1.8 Hz, 1H), 10.63 (s, 1H), 7.99-7.88 (m, 3H), 7.61 (d, J=2.7 Hz, 1H), 7.35 (s, 1H), 5.05-4.77 (m, 1H), 3.89 (s, 3H), 2.26-2.20 (m, 1H), 1.74-1.54 (m, 1H), 1.22-1.05 (m, 1H).

### Example S97. Compound 97

### Step 1: Synthesis of 1-(2-(dimethylamino)ethyl)-3-(3-(2-methoxyphenyl)-1-((2-(trimethylsilyl)ethoxy)methyl)-1H-pyrrolo[2,3-b]pyridin-6-yl)urea

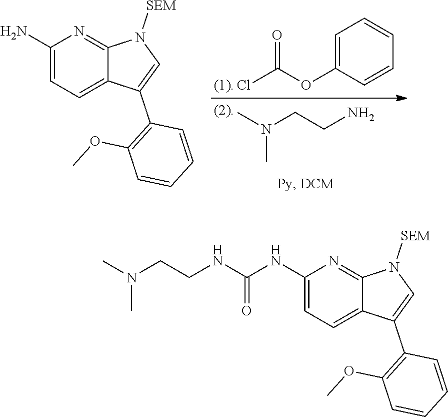

To a solution of 3-(2-methoxyphenyl)-1-((2-(trimethylsilyl)ethoxy)methyl)-1H-pyrrolo[2,3-b]pyridin-6-amine (100.0 mg, 0.27 mmol) in DCM (5.0 mL) was added Pyridine (85.6 mg, 1.08 mmol) and phenyl carbonochloridate (50.8 mg, 0.32 mmol) at 0° C. under N₂. The resulting mixture was stirred at 0° C. for 1 h. The resulting mixture was concentrated under reduced pressure. The residue was dissolved in pyridine (5.0 mL). Then (2-aminoethyl)dimethylamine (95.4 mg, 1.08 mmol) was added to the mixture at 0° C. under N₂. The resulting mixture was stirred at 60° C. for another 1 h. After the reaction was completed, the resulting mixture was concentrated under reduced pressure. The residue was purified by flash column chromatography with CH₂Cl₂/MeOH (92/8, v/v) to afford 1-(2-(dimethylamino)ethyl)-3-(3-(2-methoxyphenyl)-1-((2-(trimethylsilyl)ethoxy)methyl)-1H-pyrrolo[2,3-b]pyridin-6-yl)urea (100.0 mg, 77%) as a white solid. LCMS (ESI, m/z): [M+H]⁺=484.3.

### Step 2: Synthesis of 3-[2-(dimethylamino)ethyl]-1-[3-(2-methoxyphenyl)-1H-pyrrolo[2,3-b]pyridin-6-yl]urea (Compound 97)

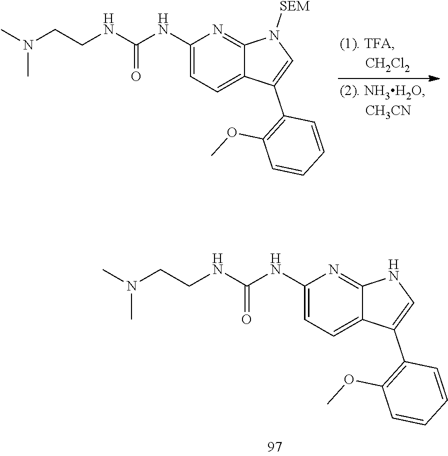 [SMILES: COc1ccccc1-c1cn(COCC[Si](C)(C)C)c2nc(NC(=O)NCCN(C)C)ccc12 ; COc1ccccc1-c1c[nH]c2nc(NC(=O)NCCN(C)C)ccc12]

To a solution of 1-(2-(dimethylamino)ethyl)-3-(3-(2-methoxyphenyl)-1-((2-(trimethylsilyl)ethoxy)methyl)-1H-pyrrolo[2,3-b]pyridin-6-yl)urea (100.0 mg, 0.21 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in ACN (3.0 mL) was added NH₃—H₂O (3.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH F-Phenyl OBD column, 19×250 mm, 5 um; Mobile Phase A: Water (10 mmol/L NH₄HCO₃), Mobile Phase B: MeOH—Preparative; Flow rate: 25 mL/min; Gradient: 70% B to 90% B in 7 min; 254 nm) to afford 3-[2-(dimethylamino)ethyl]-1-[3-(2-methoxyphenyl)-1H-pyrrolo[2,3-b]pyridin-6-yl]urea (19.7 mg, 27%) as a white solid. LCMS (ESI, m/z): [M+H]⁺=354.2. ¹H NMR (400 MHz, DMSO-d₆): δ 11.41 (s, 1H), 9.16 (s, 1H), 8.37 (s, 1H), 7.90 (d, J=8.4 Hz, 1H), 7.51-7.49 (m, 1H), 7.45 (d, J=2.4 Hz, 1H), 7.28-7.23 (m, 1H), 7.11-7.07 (m, 2H), 7.03-6.99 (m, 1H), 3.80 (s, 3H), 3.32-3.28 (m, 2H), 2.41-2.38 (m, 2H), 2.20 (s, 6H).

### Example S98. Compound 98

### Step 1: Synthesis of 6-chloro-3-(5-fluoro-2-methoxyphenyl)-1-[[2-(trimethylsilyl)ethoxy]methyl]pyrazolo[3,4-b]pyridine

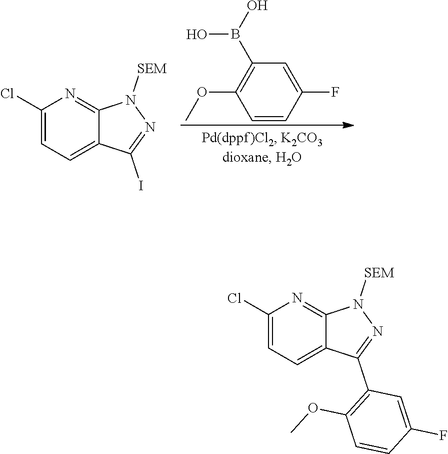

To a solution of 6-chloro-3-iodo-1-[[2-(trimethylsilyl)ethoxy]methyl]pyrazolo[3,4-b]pyridine (300.0 mg, 0.73 mmol) in dioxane/H₂O (5.0/1.0 mL) was added 5-fluoro-2-methoxyphenylboronic acid (149.3 mg, 0.88 mmol), K₂CO₃ (303.5 mg, 2.19 mmol) and Pd(dppf)Cl₂ (53.5 mg, 0.07 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 6-chloro-3-(5-fluoro-2-methoxyphenyl)-1-[[2-(trimethylsilyl)ethoxy]methyl]pyrazolo[3,4-b]pyridine (220.0 mg, 74%) as a yellow oil. LCMS (ESI, m/z): [M+H]⁺=408.1.

### Step 2: Synthesis of (1S,2S)-2-fluoro-N-[3-(5-fluoro-2-methoxyphenyl)-1-[[2-(trimethylsilyl)ethoxy]methyl]pyrazolo[3,4-b]pyridin-6-yl]cyclopropane-1-carboxamide

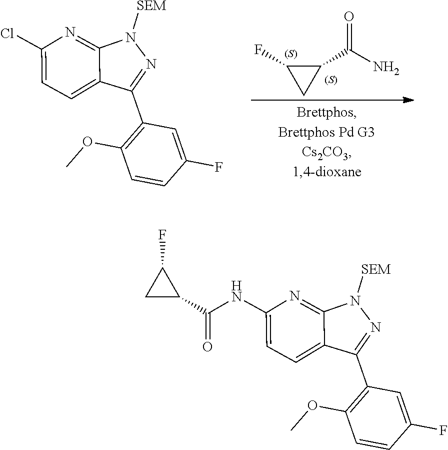

To a solution of 6-chloro-3-(5-fluoro-2-methoxyphenyl)-1-[[2-(trimethylsilyl)ethoxy]methyl]pyrazolo[3,4-b]pyridine (160.0 mg, 0.38 mmol) in 1,4-dioxane (4.0 mL) was added (1S,2S)-2-fluorocyclopropane-1-carboxamide (202.1 mg, 1.96 mmol), BrettPhos (42.1 mg, 0.08 mmol), Cs₂CO₃ (383.3 mg, 1.17 mmol) and BrettPhos Pd G3 (35.5 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford (1S,2S)-2-fluoro-N-[3-(5-fluoro-2-methoxyphenyl)-1-[[2-(trimethylsilyl)ethoxy]methyl]pyrazolo[3,4-b]pyridin-6-yl]cyclopropane-1-carboxamide (90.0 mg, 48%) as a yellow oil. LCMS (ESI, m/z): [M+H]⁺=475.2.

### Step 3: Synthesis of (1S,2S)-2-fluoro-N-[3-(5-fluoro-2-methoxyphenyl)-1H-pyrazolo[3,4-b]pyridin-6-yl]cyclopropane-1-carboxamide (Compound 98)

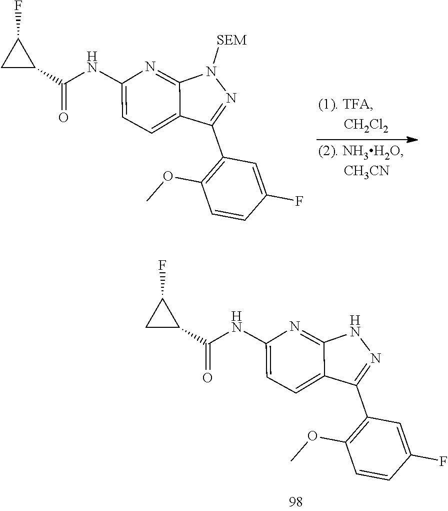 [SMILES: COc1ccc(F)cc1-c1nn(COCC[Si](C)(C)C)c2nc(NC(=O)[C@@H]3C[C@@H]3F)ccc12 ; COc1ccc(F)cc1-c1n[nH]c2nc(NC(=O)[C@@H]3C[C@@H]3F)ccc12]

To a solution of (1S,2S)-2-fluoro-N-[3-(5-fluoro-2-methoxyphenyl)-1-[[2-(trimethylsilyl)ethoxy]methyl]pyrazolo[3,4-b]pyridin-6-yl]cyclopropane-1-carboxamide (90.0 mg, 0.19 mmol) in CH₃Cl₂ (2.0 mL) was added TFA (2.0 mL) at room temperature. The resulting mixture was stirred at room temperature for 3 h. The resulting mixture was concentrated under reduced pressure. The residue was re-dissolved in ACN (2.0 mL) and NH₃·H₂O (2.0 mL). The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 34% B to 44% B in 8 min; 254 nm) to afford (1S,2S)-2-fluoro-N-[3-(5-fluoro-2-methoxyphenyl)-1H-pyrazolo[3,4-b]pyridin-6-yl]cyclopropane-1-carboxamide (12.1 mg, 18%) as a white solid. LCMS (ESI, m/z): [M+H]⁺=345.2. ¹H NMR (400 MHz, DMSO-d₆): δ 13.58 (s, 1H), 10.97 (s, 1H), 8.15 (d, J=8.8 Hz, 1H), 8.00 (d, J=8.8 Hz, 1H), 7.45 (d, J=2.4 Hz, 1H), 7.30-7.21 (m, 2H), 5.10-4.83 (m, 1H), 3.83 (s, 3H), 2.37-2.23 (m, 1H), 1.78-1.67 (m, 1H), 1.26-1.13 (m, 1H).

### Example S99. Compound 99

### Step 1: Synthesis of 6-chloro-3-(4-fluoro-2-methoxyphenyl)-1-[[2-(trimethylsilyl)ethoxy]methyl]pyrazolo[3,4-b]pyridine

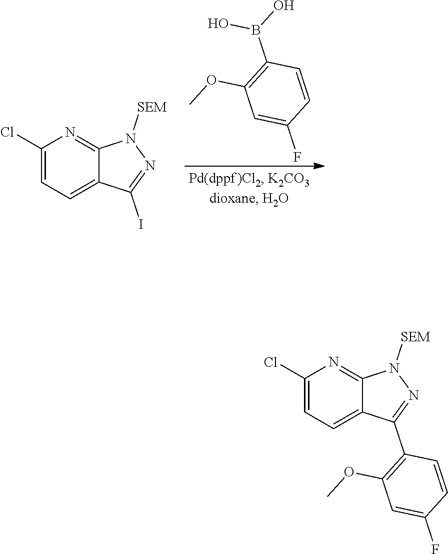 [SMILES: C[Si](C)(C)CCOCn1nc(I)c2ccc(Cl)nc21 ; COc1cc(F)ccc1B(O)O ; COc1cc(F)ccc1-c1nn(COCC[Si](C)(C)C)c2nc(Cl)ccc12]

To a solution of 6-chloro-3-iodo-1-[[2-(trimethylsilyl)ethoxy]methyl]pyrazolo[3,4-b]pyridine (500.0 mg, 1.22 mmol) in 1,4-dioxane/H₂O (25.0 mL/5.0 mL) was added 4-fluoro-2-methoxyphenylboronic acid (207.4 mg, 1.22 mmol), K₂CO₃ (337.3 mg, 2.44 mmol) and Pd(dppf)Cl₂ (89.3 mg, 0.12 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(4-fluoro-2-methoxyphenyl)-1-[[2-(trimethylsilyl)ethoxy]methyl]pyrazolo[3,4-b]pyridine (347.0 mg, 70%) as a light yellow oil. LCMS (ESI, m/z): [M+H]⁺=408.1.

### Step 2: Synthesis of (1S,2S)-2-fluoro-N-[3-(4-fluoro-2-methoxyphenyl)-1-[[2-(trimethylsilyl)ethoxy]methyl]pyrazolo[3,4-b]pyridin-6-yl]cyclopropane-1-carboxamide

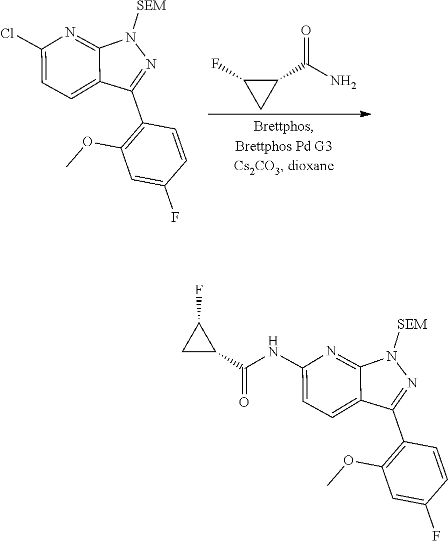 [SMILES: COc1cc(F)ccc1-c1nn(COCC[Si](C)(C)C)c2nc(Cl)ccc12 ; NC(=O)[C@@H]1C[C@@H]1F ; COc1cc(F)ccc1-c1nn(COCC[Si](C)(C)C)c2nc(NC(=O)[C@@H]3C[C@@H]3F)ccc12]

To a solution of 6-chloro-3-(4-fluoro-2-methoxyphenyl)-1-[[2-(trimethylsilyl)ethoxy]methyl]pyrazolo[3,4-b]pyridine (347.0 mg, 0.85 mmol) in 1,4-dioxane (20.0 mL) was added (1S,2S)-2-fluorocyclopropane-1-carboxamide (438.5 mg, 4.26 mmol), BrettPhos (91.3 mg, 0.17 mmol), Cs₂CO₃ (831.4 mg, 2.55 mmol) and BrettPhos Pd G3 (77.1 mg, 0.09 mmol) at room temperature under N₂. The resulting mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(4-fluoro-2-methoxyphenyl)-1-[[2-(trimethylsilyl)ethoxy]methyl]pyrazolo[3,4-b]pyridin-6-yl]cyclopropane-1-carboxamide (56.5 mg, 14%) as a light yellow oil. LCMS (ESI, m/z): [M+H]⁺=475.2.

### Step 3: Synthesis of (1S,2S)-2-fluoro-N-[3-(4-fluoro-2-methoxyphenyl)-1H-pyrazolo[3,4-b]pyridin-6-yl]cyclopropane-1-carboxamide (Compound 99)

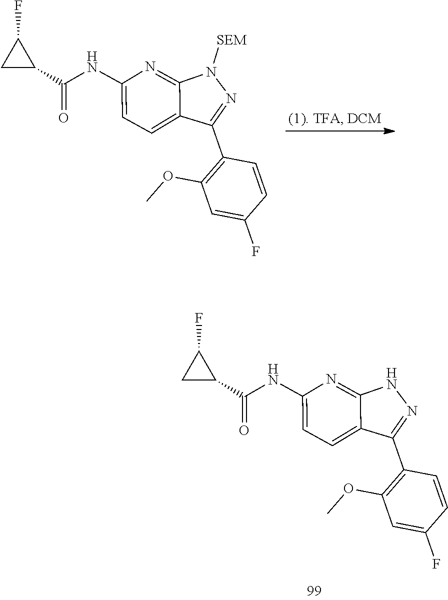 [SMILES: COc1cc(F)ccc1-c1nn(COCC[Si](C)(C)C)c2nc(NC(=O)[C@@H]3C[C@@H]3F)ccc12 ; COc1cc(F)ccc1-c1n[nH]c2nc(NC(=O)[C@@H]3C[C@@H]3F)ccc12]

To a solution of (1S,2S)-2-fluoro-N-[3-(4-fluoro-2-methoxyphenyl)-1-[[2-(trimethylsilyl)ethoxy]methyl]pyrazolo[3,4-b]pyridin-6-yl]cyclopropane-1-carboxamide (56.6 mg, 0.12 mmol) in DCM (2.0 mL) was added TFA (2.0 mL) at room temperature. The resulting mixture was stirred at room temperature for 4 h. After the reaction was completed, the resulting mixture was diluted with H₂O. The pH value of the mixture was adjusted to 7 with aq·NaHCO₃. The mixture was extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 36% B to 54% B in 8 min; 254/220 nm) to afford (1S,2S)-2-fluoro-N-[3-(4-fluoro-2-methoxyphenyl)-1H-pyrazolo[3,4-b]pyridin-6-yl]cyclopropane-1-carboxamide (6.8 mg, 16%) as a white solid. LCMS (ESI, m/z): [M+H]⁺=345.3. ¹H NMR (400 MHz, DMSO-d₆): δ 13.50 (s, 1H), 10.96 (s, 1H), 8.10 (d, J=8.8 Hz, 1H), 7.98 (d, J=8.8 Hz, 1H), 7.65-7.61 (m, 1H), 7.12-7.08 (m, 1H), 6.92-6.87 (m, 1H), 5.04-4.86 (m, 1H), 3.85 (s, 3H), 2.35-2.25 (m, 1H), 1.70-1.64 (m, 1H), 1.22-1.16 (m, 1H).

### Example S100. Compound 100

### Step 1: Synthesis of 6-chloro-3-(3-fluoro-2-methoxyphenyl)-1-((2-(trimethylsilyl)ethoxy)methyl)-1H-pyrazolo[3,4-b]pyridine

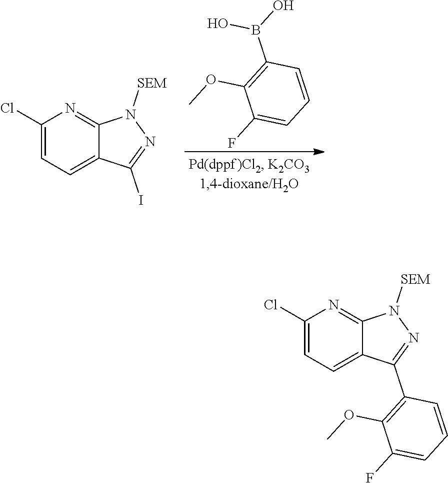 [SMILES: C[Si](C)(C)CCOCn1nc(I)c2ccc(Cl)nc21 ; COc1c(F)cccc1B(O)O ; COc1c(F)cccc1-c1nn(COCC[Si](C)(C)C)c2nc(Cl)ccc12]

To a solution of 6-chloro-3-iodo-1-((2-(trimethylsilyl)ethoxy)methyl)-1H-pyrazolo[3,4-b]pyridine (400.0 mg, 0.98 mmol) in 1,4-dioxane/H₂O (8.0/2.0 mL) was added (3-fluoro-2-methoxyphenyl)boronic acid (165.9 mg, 0.98 mmol), K₂CO₃ (404.8 mg, 2.93 mmol) and Pd(dppf)Cl₂ (71.4 mg, 0.10 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous Na₂SO₄ and filtered. The filtrate was concentrated under reduced pressure. The residue was purified by flash column chromatography with petroleum ether/ethyl acetate (80/20, v/v) to afford 6-chloro-3-(3-fluoro-2-methoxyphenyl)-1-((2-(trimethylsilyl)ethoxy)methyl)-1H-pyrazolo[3,4-b]pyridine (370.0 mg, 88%) as a light yellow oil. LCMS (ESI, m/z): [M+H]⁺=408.1.

### Step 2: Synthesis of (1S,2S)-2-fluoro-N-(3-(3-fluoro-2-methoxyphenyl)-1-((2-(trimethylsilyl)ethoxy)methyl)-1H-pyrazolo[3,4-b]pyridin-6-yl)cyclopropane-1-carboxamide

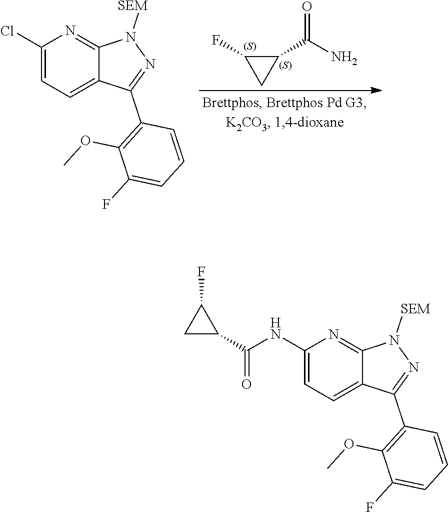

To a solution of 6-chloro-3-(3-fluoro-2-methoxyphenyl)-1-((2-(trimethylsilyl)ethoxy)methyl)-1H-pyrazolo[3,4-b]pyridine (310.0 mg, 0.76 mmol) in 1,4-dioxane (5.0 mL) was added (1S,2S)-2-fluorocyclopropane-1-carboxamide (391.7 mg, 3.80 mmol), K₂CO₃ (315.1 mg, 2.28 mmol), Brettphos (81.6 mg, 0.15 mmol) and Brettphos Pd G3 (68.9 mg, 0.08 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (83/17, v/v) to afford (1S,2S)-2-fluoro-N-(3-(3-fluoro-2-methoxyphenyl)-1-((2-(trimethylsilyl)ethoxy)methyl)-1H-pyrazolo[3,4-b]pyridin-6-yl)cyclopropane-1-carboxamide (106.0 mg, 28%) as a brown oil. LCMS (ESI, m/z): [M+H]⁺=475.2.

### Step 3: Synthesis of (1S,2S)-2-fluoro-N-(3-(3-fluoro-2-methoxyphenyl)-1H-pyrazolo[3,4-b]pyridin-6-yl)cyclopropane-1-carboxamide (Compound 100)

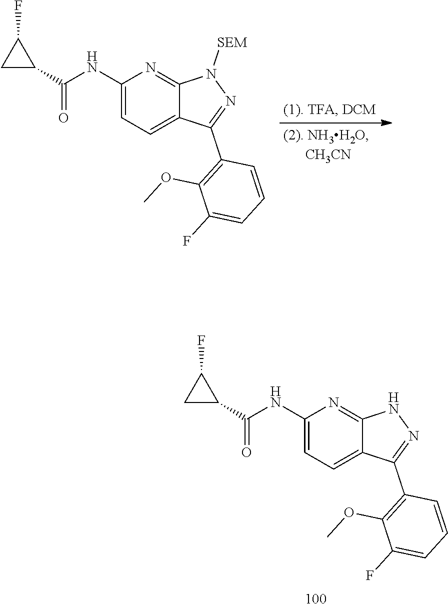 [SMILES: COc1c(F)cccc1-c1nn(COCC[Si](C)(C)C)c2nc(NC(=O)[C@@H]3C[C@@H]3F)ccc12 ; COc1c(F)cccc1-c1n[nH]c2nc(NC(=O)[C@@H]3C[C@@H]3F)ccc12]

To a solution of (1S,2S)-2-fluoro-N-(3-(3-fluoro-2-methoxyphenyl)-1-((2-(trimethylsilyl)ethoxy)methyl)-1H-pyrazolo[3,4-b]pyridin-6-yl)cyclopropane-1-carboxamide (86.0 mg, 0.18 mmol) in DCM (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 34% B to 51% B in 7 min; 254 nm) to afford (1S,2S)-2-fluoro-N-(3-(3-fluoro-2-methoxyphenyl)-1H-pyrazolo[3,4-b]pyridin-6-yl)cyclopropane-1-carboxamide (12.2 mg, 19%) as a light yellow solid. LCMS (ESI, m/z): [M+H]⁺=345.2. ¹H NMR (300 MHz, DMSO-d₆): δ 13.67 (s, 1H), 11.02 (s, 1H), 8.21 (d, J=9.0 Hz, 1H), 8.03 (d, J=9.0 Hz, 1H), 7.52 (d, J=7.8 Hz, 1H), 7.42-7.35 (m, 1H), 7.28-7.21 (m, 1H), 5.09-4.83 (m, 1H), 3.70 (d, J=1.2 Hz, 3H), 2.47-2.32 (m, 1H), 1.74-1.61 (m, 1H), 1.26-1.16 (m, 1H).

### Example S101. Compound 101

### Step 1: Synthesis of 2-(6-chloro-1-((2-(trimethylsilyl)ethoxy)methyl)-1H-pyrazolo[3,4-b]pyridin-3-yl)phenol

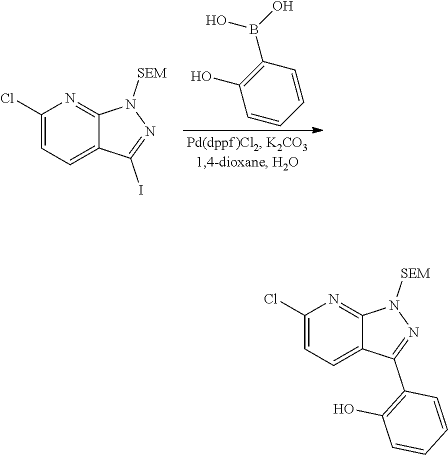 [SMILES: C[Si](C)(C)CCOCn1nc(I)c2ccc(Cl)nc21 ; OB(O)c1ccccc1O ; C[Si](C)(C)CCOCn1nc(-c2ccccc2O)c2ccc(Cl)nc21]

To a solution of 6-chloro-3-iodo-1-((2-(trimethylsilyl)ethoxy)methyl)-1H-pyrazolo[3,4-b]pyridine (400.0 mg, 0.97 mmol) in 1,4-dioxane/H₂O (10.0 mL/2.0 mL) was added (2-hydroxyphenyl)boronic acid (134.7 mg, 0.97 mmol), K₂CO₃ (404.8 mg, 2.93 mmol) and Pd(dppf)Cl₂ (71.4 mg, 0.10 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford 2-(6-chloro-1-((2-(trimethylsilyl)ethoxy)methyl)-1H-pyrazolo[3,4-b]pyridin-3-yl)phenol (270.0 mg, 74%) as a light green solid. LCMS (ESI, m/z): [M+H]⁺=376.1.

### Step 2: Synthesis of 6-chloro-3-(2-(methoxy-d3)phenyl)-1-((2-(trimethylsilyl)ethoxy)methyl)-1H-pyrazolo[3,4-b]pyridine

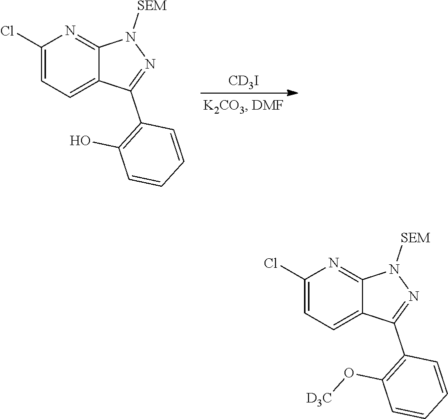 [SMILES: C[Si](C)(C)CCOCn1nc(-c2ccccc2O)c2ccc(Cl)nc21 ; "CD3I" ; [2H]C([2H])([2H])Oc1ccccc1-c1nn(COCC[Si](C)(C)C)c2nc(Cl)ccc12]

To a solution of 2-(6-chloro-1-((2-(trimethylsilyl)ethoxy)methyl)-1H-pyrazolo[3,4-b]pyridin-3-yl)phenol (220.0 mg, 0.59 mmol) in DMF (5.0 mL) was added K₂CO₃ (242.6 mg, 1.76 mmol) and iodomethane-d3 (127.3 mg, 0.88 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (99/1, v/v) to afford 6-chloro-3-(2-(methoxy-d3)phenyl)-1-((2-(trimethylsilyl)ethoxy)methyl)-1H-pyrazolo[3,4-b]pyridine (210.0 mg, 91%) as a colorless oil. LCMS (ESI, m/z): [M+H]⁺=393.2.

### Step 3: Synthesis of (1S,2S)-2-fluoro-N-(3-(2-(methoxy-d3)phenyl)-1-((2-(trimethylsilyl)ethoxy)methyl)-1H-pyrazolo[3,4-b]pyridin-6-yl)cyclopropane-1-carboxamide

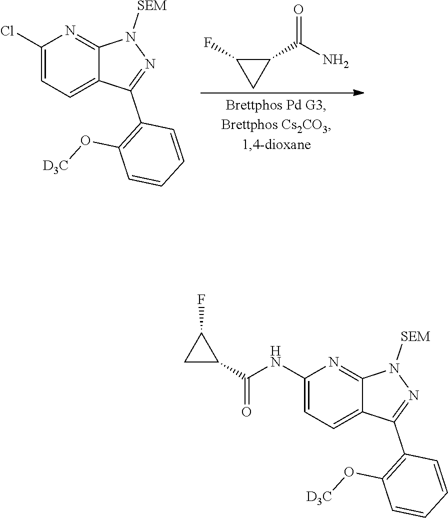 [SMILES: [2H]C([2H])([2H])Oc1ccccc1-c1nn(COCC[Si](C)(C)C)c2nc(Cl)ccc12 ; NC(=O)[C@@H]1C[C@@H]1F ; [2H]C([2H])([2H])Oc1ccccc1-c1nn(COCC[Si](C)(C)C)c2nc(NC(=O)[C@@H]3C[C@@H]3F)ccc12]

To a solution of 6-chloro-3-(2-(methoxy-d3)phenyl)-1-((2-(trimethylsilyl)ethoxy)methyl)-1H-pyrazolo[3,4-b]pyridine (120.0 mg, 0.31 mmol) in 1,4-dioxane (6.0 mL) was added (1S,2S)-2-fluorocyclopropane-1-carboxamide (157.4 mg, 1.53 mmol), Cs₂CO₃ (298.5 mg, 0.92 mmol), BrettPhos (32.8 mg, 0.06 mmol) and BrettPhos Pd G3 (27.7 mg, 0.03 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford (1S,2S)-2-fluoro-N-(3-(2-(methoxy-d3)phenyl)-1-((2-(trimethylsilyl)ethoxy)methyl)-1H-pyrazolo[3,4-b]pyridin-6-yl)cyclopropane-1-carboxamide (75.0 mg, 53%) as a yellow solid. LCMS (ESI, m/z): [M+H]⁺=460.2.

### Step 4: Synthesis of (1S,2S)-2-fluoro-N-(3-(2-(methoxy-d3)phenyl)-1H-pyrazolo[3,4-b]pyridin-6-yl)cyclopropane-1-carboxamide (Compound 101)

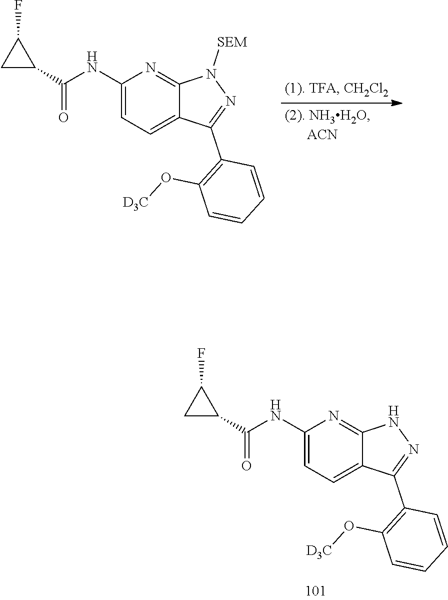 [SMILES: [2H]C([2H])([2H])Oc1ccccc1-c1nn(COCC[Si](C)(C)C)c2nc(NC(=O)[C@@H]3C[C@@H]3F)ccc12 ; [2H]C([2H])([2H])Oc1ccccc1-c1n[nH]c2nc(NC(=O)[C@@H]3C[C@@H]3F)ccc12]

To a solution of (1S,2S)-2-fluoro-N-(3-(2-(methoxy-d3)phenyl)-1-((2-(trimethylsilyl)ethoxy)methyl)-1H-pyrazolo[3,4-b]pyridin-6-yl)cyclopropane-1-carboxamide (75.0 mg, 0.16 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in ACN (5.0 mL) was added NH₃·H₂O (5.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 19×250 mm, 10 um; Mobile Phase A: Water (0.1% FA), Mobile Phase B: ACN; Flow rate: 25 mL/min; Gradient: 39% B to 49% B in 10 min; 254 nm) to afford (1S,2S)-2-fluoro-N-(3-(2-(methoxy-d3)phenyl)-1H-pyrazolo[3,4-b]pyridin-6-yl)cyclopropane-1-carboxamide (10.9 mg, 20%) as a white solid. LCMS (ESI, m/z): [M+H]⁺=330.1. ¹H NMR (300 MHz, DMSO-d₆): δ 13.47 (s, 1H), 10.97 (s, 1H), 8.12 (d, J=9.0 Hz, 1H), 7.99 (d, J=9.0 Hz, 1H), 7.64-7.61 (m, 1H), 7.47-7.41 (m, 1H), 7.19 (d, J=7.5 Hz, 1H), 7.10-7.05 (m, 1H), 5.08-4.82 (m, 1H), 2.30-2.25 (m, 1H), 1.73-1.62 (m, 1H), 1.23-1.16 (m, 1H).

### Example S102. Compound 102

### Step 1: Synthesis of 5-(6-chloro-1-[[2-(trimethylsilyl)ethoxy]methyl]pyrrolo[2,3-b]pyridin-3-yl)-4,6-dimethoxypyrimidine

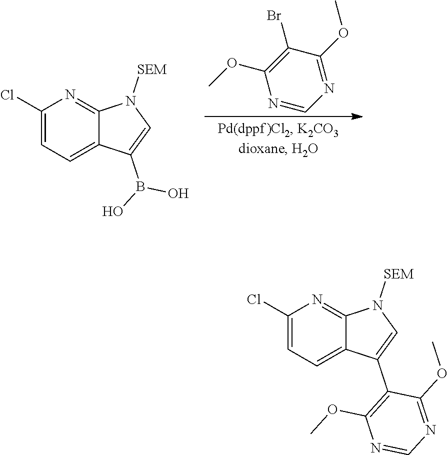 [SMILES: C[Si](C)(C)CCOCn1cc(B(O)O)c2ccc(Cl)nc21 ; COc1ncnc(OC)c1Br ; COc1ncnc(OC)c1-c1cn(COCC[Si](C)(C)C)c2nc(Cl)ccc12]

To a solution of 6-chloro-1-[[2-(trimethylsilyl)ethoxy]methyl]pyrrolo[2,3-b]pyridin-3-ylboronic acid (350.0 mg, 1.07 mmol) in dioxane/H₂O (5.0/1.0 mL) was added 5-bromo-4,6-dimethoxypyrimidine (187.7 mg, 0.85 mmol), K₂CO₃ (444.2 mg, 3.20 mmol) and Pd(dppf)Cl₂ (78.4 mg, 0.10 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 5-(6-chloro-1-[[2-(trimethylsilyl)ethoxy]methyl]pyrrolo[2,3-b]pyridin-3-yl)-4,6-dimethoxypyrimidine (180.0 mg, 40%) as a white solid. LCMS (ESI, m/z): [M+H]⁺=421.1.

### Step 2: Synthesis of (1S,2S)—N-[3-(4,6-dimethoxypyrimidin-5-yl)-1-[[2-(trimethylsilyl)ethoxy]methyl]pyrrolo[2,3-b]pyridin-6-yl]-2-fluorocyclopropane-1-carboxamide

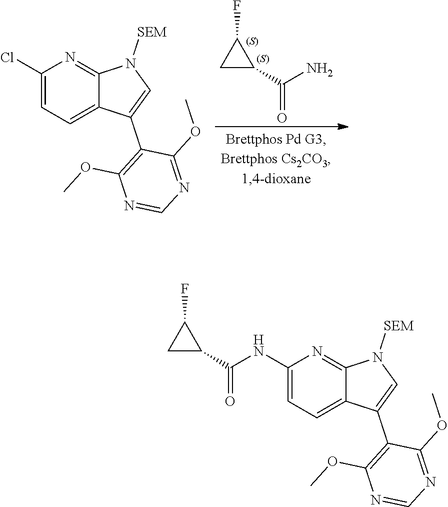

To a solution of 5-(6-chloro-1-[[2-(trimethylsilyl)ethoxy]methyl]pyrrolo[2,3-b]pyridin-3-yl)-4,6-dimethoxypyrimidine (160.0 mg, 0.38 mmol) in 1,4-dioxane (4.0 mL) was added (1S,2S)-2-fluorocyclopropane-1-carboxamide (195.9 mg, 1.90 mmol), Cs₂CO₃ (371.5 mg, 1.14 mmol), BrettPhos (40.8 mg, 0.07 mmol) and BrettPhos Pd G3 (34.4 mg, 0.04 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (70/30, v/v) to afford (1S,2S)—N-[3-(4,6-dimethoxypyrimidin-5-yl)-1-[[2-(trimethylsilyl)ethoxy]methyl]pyrrolo[2,3-b]pyridin-6-yl]-2-fluorocyclopropane-1-carboxamide (160.0 mg, 86%) as a white solid. LCMS (ESI, m/z): [M+H]⁺=488.2.

### Step 3: Synthesis of (1S,2S)—N-[3-(4,6-dimethoxypyrimidin-5-yl)-1H-pyrrolo[2,3-b]pyridin-6-yl]-2-fluorocyclopropane-1-carboxamide (Compound 102)

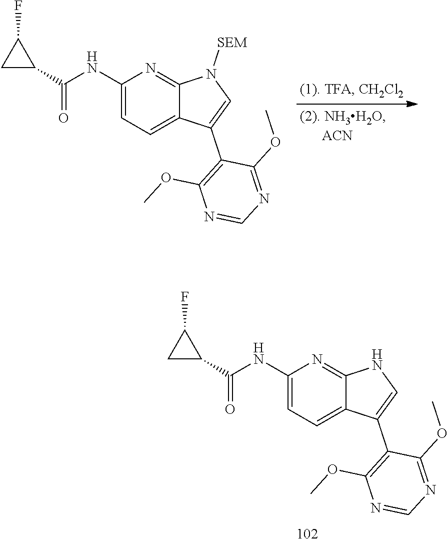 [SMILES: COc1ncnc(OC)c1-c1cn(COCC[Si](C)(C)C)c2nc(NC(=O)[C@@H]3C[C@@H]3F)ccc12 ; COc1ncnc(OC)c1-c1c[nH]c2nc(NC(=O)[C@@H]3C[C@@H]3F)ccc12]

To a solution of (1S,2S)—N-[3-(4,6-dimethoxypyrimidin-5-yl)-1-[[2-(trimethylsilyl)ethoxy]methyl]pyrrolo[2,3-b]pyridin-6-yl]-2-fluorocyclopropane-1-carboxamide (280.0 mg, 0.44 mmol) in CH₂Cl₂ (2.0 mL) was added TFA (2.0 mL) at room temperature. The mixture was stirred at room temperature for 2 h. The resulting mixture was concentrated under reduced pressure. To the residue in ACN (2.0 mL) was added NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 31% B to 40% B in 9 min; 254 nm) to afford (1S,2S)—N-[3-(4,6-dimethoxypyrimidin-5-yl)-1H-pyrrolo[2,3-b]pyridin-6-yl]-2-fluorocyclopropane-1-carboxamide (29.5 mg, 25%) as a white solid. LCMS (ESI, m/z): [M+H]⁺=358.1. ¹H NMR (400 MHz, DMSO-d₆): δ 11.66 (s, 1H), 10.62 (s, 1H), 8.47 (s, 1H), 7.86 (d, J=8.4 Hz, 1H), 7.71 (d, J=8.4 Hz, 1H), 7.49 (d, J=1.6 Hz, 1H), 5.01-4.82 (m, 1H), 3.91 (s, 6H), 2.27-2.19 (m, 1H), 1.69-1.61 (m, 1H), 1.17-1.14 (m, 1H).

### Example S103. Compound 103

### Step 1: Synthesis of 3-ethyl-1-[3-(2-methoxyphenyl)-1-[[2-(trimethylsilyl)ethoxy]methyl]pyrrolo[2,3-b]pyridin-6-yl]urea

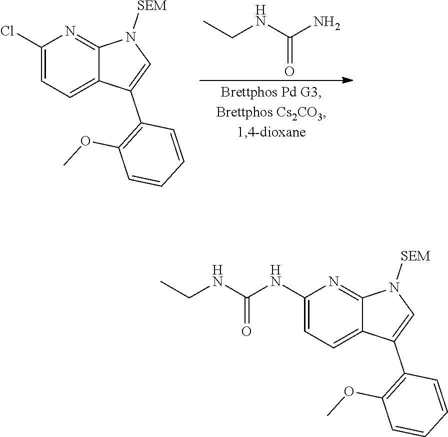

To a mixture of 6-chloro-3-(2-methoxyphenyl)-1-[[2-(trimethylsilyl)ethoxy]methyl]pyrrolo[2,3-b]pyridine (300.0 mg, 0.77 mmol) and ethylurea (206.0 mg, 0.23 mmol) in dioxane (5.0 mL) was added Brettphos Pd G3 (69.9 mg, 0.07 mmol), Cs₂CO₃ (753.8 mg, 2.31 mmol) and BrettPhos (84.0 mg, 0.14 mmol) at room temperature under N₂. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-ethyl-1-[3-(2-methoxyphenyl)-1-[[2-(trimethylsilyl) ethoxy]methyl]pyrrolo[2,3-b]pyridin-6-yl]urea (300.0 mg, 88%) as a yellow solid. LCMS (ESI, m/z): [M+H]⁺=441.2

### Step 2: Synthesis of 3-ethyl-1-[3-(2-methoxyphenyl)-1H-pyrrolo[2,3-b]pyridin-6-yl]urea (Compound 103)

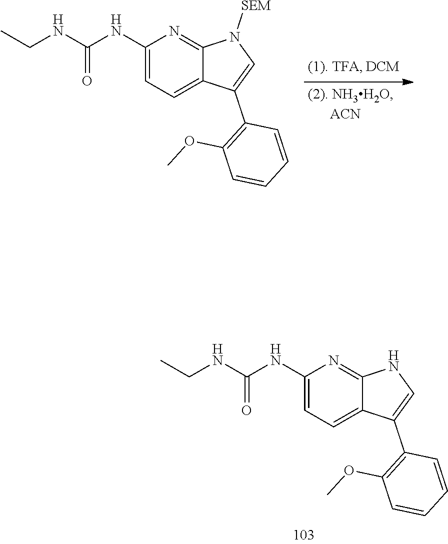 [SMILES: CCNC(=O)Nc1ccc2c(-c3ccccc3OC)cn(COCC[Si](C)(C)C)c2n1 ; CCNC(=O)Nc1ccc2c(-c3ccccc3OC)c[nH]c2n1]

To a solution of 3-ethyl-1-[3-(2-methoxyphenyl)-1-[[2-(trimethylsilyl)ethoxy]methyl] pyrrolo[2,3-b]pyridin-6-yl]urea (170.0 mg, 0.38 mmol) in DCM (2.0 mL) was added TFA (1.0 mL) at room temperature. The resulting mixture was stirred at room temperature for 1 h. The resulting mixture was concentrated under reduced pressure. To the above residue was added ACN (1.0 mL) and NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18 30×250 mm, 5 um; Mobile Phase A: Water (10 mmol/L NH₄HCO₃), Mobile Phase B: ACN; Flow rate: 60 mL/min; Gradient: 41% B to 50% B in 9 min; 254 nm) to afford 3-ethyl-1-[3-(2-methoxyphenyl)-1H-pyrrolo[2,3-b]pyridin-6-yl]urea (43.9 mg, 36%) as a white solid. LCMS (ESI, m/z): [M+H]⁺=311.2. ¹H NMR (300 MHz, DMSO-d₆): δ 11.51 (s, 1H), 9.15 (s, 1H), 8.59-8.56 (m, 1H), 7.91 (d, J=8.4 Hz, 1H), 7.52-7.46 (m, 2H), 7.29-7.23 (m, 1H), 7.10 (d, J=7.5 Hz, 1H), 7.04-6.99 (m, 2H), 3.81 (s, 3H), 3.30-3.21 (m, 2H), 1.19-1.14 (m, 3H).

### Example S104. Compound 104

### Step 1: Synthesis of 3-(2-cyanoethyl)-1-[3-(2-methoxyphenyl)-1-[[2-(trimethylsilyl)ethoxy]methyl]pyrrolo[2,3-b]pyridin-6-yl]urea

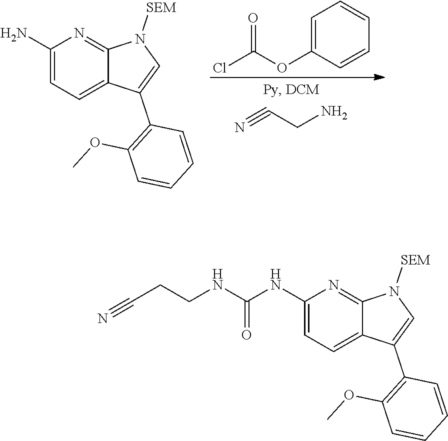

To a solution of 3-(2-methoxyphenyl)-1-[[2-(trimethylsilyl)ethoxy]methyl]pyrrolo[2,3-b]pyridin-6-amine (100.0 mg, 0.27 mmol) in CH₂Cl₂ (2.0 mL) was added Pyridine (85.6 mg, 1.08 mmol) and phenyl chloroformate (50.8 mg, 0.33 mmol) at 0° C. The mixture was stirred at room temperature for 16 h. The mixture was concentrated under vacuum. To the residue was added pyridine (2.0 mL) and 3-aminopropanenitrile (56.9 mg, 0.81 mmol). The resulting mixture was stirred at 60° C. for another 2 h. After the reaction was completed, the resulting mixture was concentrated under vacuum. The residue was purified by flash column chromatography with petroleum ether/ethyl acetate (20/80, v/v) to afford 3-(2-cyanoethyl)-1-[3-(2-methoxyphenyl)-1-[[2-(trimethylsilyl)ethoxy]methyl]pyrrolo[2,3-b]pyridin-6-yl]urea (70.0 mg, 55%) as a yellow solid. LCMS (ESI, m/z): [M+H]⁺=466.2.

### Step 2: Synthesis of 3-(2-cyanoethyl)-1-[3-(2-methoxyphenyl)-1H-pyrrolo[2,3-b]pyridin-6-yl]urea (Compound 104)

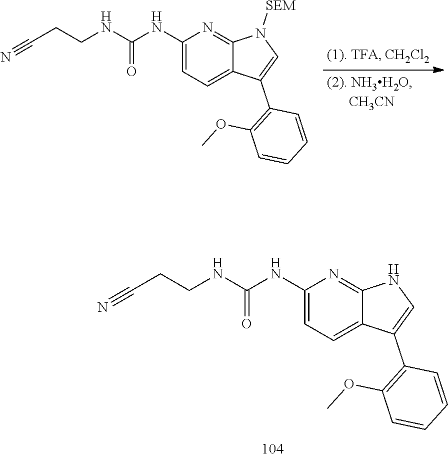 [SMILES: COc1ccccc1-c1cn(COCC[Si](C)(C)C)c2nc(NC(=O)NCCC#N)ccc12 ; COc1ccccc1-c1c[nH]c2nc(NC(=O)NCCC#N)ccc12]

To a solution of 3-(2-cyanoethyl)-1-[3-(2-methoxyphenyl)-1-[[2-(trimethylsilyl)ethoxy]methyl]pyrrolo[2,3-b]pyridin-6-yl]urea (60.0 mg, 0.13 mmol) in CH₂Cl₂ (1.0 mL) was added TFA (1.0 mL). The mixture was stirred at room temperature for 2 h. The mixture was concentrated under vacuum. The residue was dissolved in ACN (1.0 mL) and NH₃·H₂O (1.0 mL). The resulting mixture was stirred at room temperature for another 2 h. After the reaction was completed, the resulting mixture was concentrated under vacuum. The residue was purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 32% B to 47% B in 7 min; 254 nm) to afford 3-(2-cyanoethyl)-1-[3-(2-methoxyphenyl)-1H-pyrrolo[2,3-b]pyridin-6-yl]urea (24.5 mg, 56%) as a white solid. LCMS (ESI, m/z): [M+H]⁺=336.1. ¹H NMR (300 MHz, DMSO-d₆): δ 11.48 (s, 1H), 9.33 (s, 1H), 8.70 (s, 1H), 7.93 (d, J=8.4 Hz, 1H), 7.53-7.48 (m, 2H), 7.29-7.24 (m, 1H), 7.12-7.08 (m, 2H), 7.04-6.99 (m, 1H), 3.81 (s, 3H), 3.51-3.46 (m, 2H), 2.80-2.73 (m, 2H).

### Example S105. Compound 105

### Step 1: Synthesis of cis-3-oxa-bicyclo[4.1.0]heptan-2-one

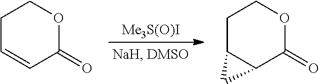 [SMILES: "Me3S(O)I" ; O=C1C=CCCO1 ; O=C1OCC[C@H]2C[C@@H]12]

To a solution of trimethylsulfoxonium iodide (1.2 g, 5.61 mmol) in DMSO (10.0 mL) was added NaH (224.6 mg, 60%) at 0° C. under N₂. The resulting mixture was stirred at 0° C. for 1 h. Then 5,6-dihydropyran-2-one (500.0 mg, 5.10 mmol) was added dropwise to the mixture at 0° C. The resulting mixture was stirred at room temperature for another 16 h under N₂. After the reaction was completed, the resulting mixture was quenched with water and extracted with Et₂O. The combined organic layer was washed with brine, dried over anhydrous sodium sulfate and filtered. The filtrate was concentrated under reduced pressure to afford cis-3-oxa-bicyclo[4.1.0]heptan-2-one (80.0 mg, crude) as a yellow oil.

### Step 2: Synthesis of cis-2-(2-hydroxyethyl)-N-(3-(2-methoxyphenyl)-1-((2-(trimethylsilyl)ethoxy)methyl)-1H-pyrrolo[2,3-b]pyridin-6-yl)cyclopropanecarboxamide

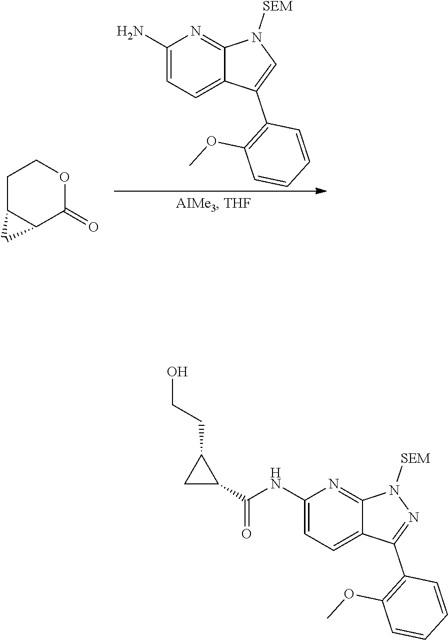 [SMILES: O=C1OCC[C@H]2C[C@@H]12 ; COc1ccccc1-c1cn(COCC[Si](C)(C)C)c2nc(N)ccc12 ; COc1ccccc1-c1nn(COCC[Si](C)(C)C)c2nc(NC(=O)[C@@H]3C[C@@H]3CCO)ccc12]

To a mixture of 3-(2-methoxyphenyl)-1-((2-(trimethylsilyl)ethoxy)methyl)-1H-pyrrolo[2,3-b]pyridin-6-amine (70.0 mg, 0.19 mmol) and cis-3-oxa-bicyclo[4.1.0]heptan-2-one (63.7 mg, crude) in THE (10.0 mL) was added AlMe₃ (0.47 mL, 2 mol/L) at 0° C. under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (30/70, v/v) to afford cis-2-(2-hydroxyethyl)-N-(3-(2-methoxyphenyl)-1-((2-(trimethylsilyl)ethoxy)methyl)-1H-pyrrolo[2,3-b]pyridin-6-yl)cyclopropanecarboxamide (40.0 mg, 44%) as a yellow solid. LCMS (ESI, m/z): [M+H]⁺=482.2.

### Step 3: Synthesis of cis-2-(2-hydroxyethyl)-N-(3-(2-methoxyphenyl)-1H-pyrrolo[2,3-b]pyridin-6-yl)cyclopropanecarboxamide (Compound 105)

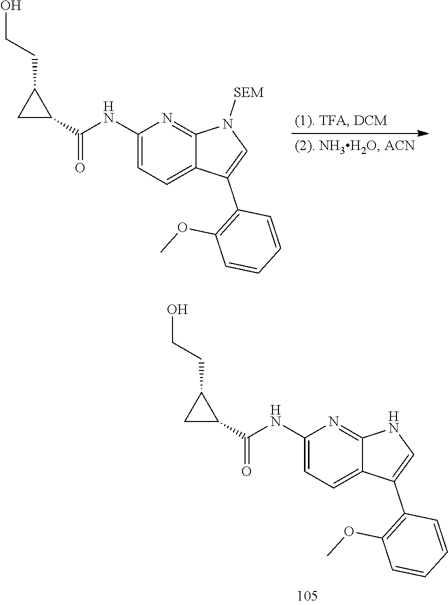 [SMILES: COc1ccccc1-c1cn(COCC[Si](C)(C)C)c2nc(NC(=O)[C@@H]3C[C@@H]3CCO)ccc12 ; COc1ccccc1-c1c[nH]c2nc(NC(=O)[C@@H]3C[C@@H]3CCO)ccc12]

To a solution of cis-2-(2-hydroxyethyl)-N-(3-(2-methoxyphenyl)-1-((2-(trimethylsilyl)ethoxy)methyl)-1H-pyrrolo[2,3-b]pyridin-6-yl)cyclopropanecarboxamide (35.0 mg, 0.07 mmol) in CH₂Cl₂ (1.0 mL) was added TFA (1.0 mL) at room temperature. The resulting mixture was stirred at room temperature for 2 h. The resulting mixture was concentrated under reduced pressure. The residue was dissolved in ACN (1.0 mL) and NH₃·H₂O (1.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with petroleum MeOH/H₂O (70/30, v/v) to afford cis-2-(2-hydroxyethyl)-N-(3-(2-methoxyphenyl)-1H-pyrrolo[2,3-b]pyridin-6-yl)cyclopropanecarboxamide (10.3 mg, 40%) as a light yellow solid. LCMS (ESI, m/z): [M+H]⁺=352.2. ¹H NMR (300 MHz, DMSO-d₆): δ 11.53 (d, J=1.8 Hz, 1H), 10.47 (s, 1H), 7.98-7.88 (m, 2H), 7.56-7.52 (m, 2H), 7.30-7.24 (m, 1H), 7.13-7.05 (m, 1H), 7.03-7.00 (m, 1H), 4.44-4.40 (m, 1H), 3.82 (s, 3H), 3.43-3.37 (m, 2H), 2.07-2.01 (m, 1H), 1.72-1.63 (m, 2H), 1.33-1.25 (m, 1H), 0.99-0.85 (m, 2H).

### Example S106. Compound 106

### Step 1: Synthesis of cis-2-cyano-N-[3-(2-methoxyphenyl)-1-[[2-(trimethylsilyl)ethoxy]methyl]pyrrolo[2,3-b]pyridin-6-yl]cyclopropane-1-carboxamide

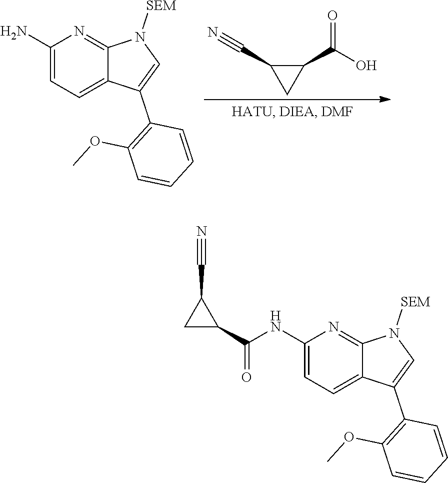 [SMILES: COc1ccccc1-c1cn(COCC[Si](C)(C)C)c2nc(N)ccc12 ; N#C[C@@H]1C[C@@H]1C(=O)O ; COc1ccccc1-c1cn(COCC[Si](C)(C)C)c2nc(NC(=O)[C@H]3C[C@H]3C#N)ccc12]

To a solution of 3-(2-methoxyphenyl)-1-[[2-(trimethylsilyl)ethoxy]methyl]pyrrolo[2,3-b]pyridin-6-amine (300.0 mg, 0.82 mmol) in DMF (10.0 mL) was added cis-2-cyanocyclopropane-1-carboxylic acid (90.9 mg, 0.81 mmol), HATU (463.2 mg, 1.21 mmol) and DIEA (157.3 mg, 1.28 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (1/1, v/v) to afford cis-2-cyano-N-[3-(2-methoxyphenyl)-1-[[2-(trimethylsilyl)ethoxy]methyl]pyrrolo[2,3-b]pyridin-6-yl]cyclopropane-1-carboxamide (300.0 mg, 80%) as a colorless oil. LCMS (ESI, m/z): [M+H]⁺=463.2.

### Step 2: Synthesis of cis-2-cyano-N-[3-(2-methoxyphenyl)-1H-pyrrolo[2,3-b]pyridin-6-yl]cyclopropane-1-carboxamide (Compound 106)

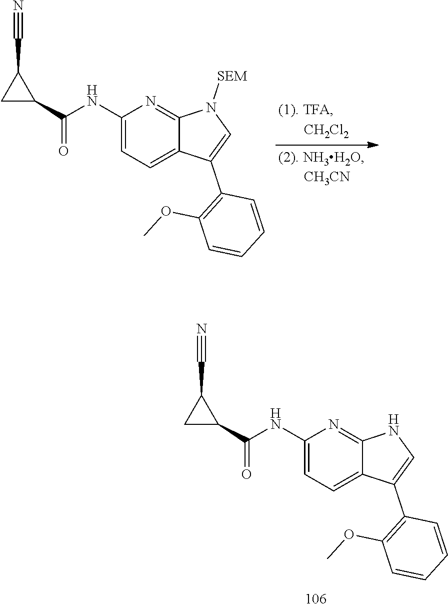 [SMILES: COc1ccccc1-c1cn(COCC[Si](C)(C)C)c2nc(NC(=O)[C@H]3C[C@H]3C#N)ccc12 ; COc1ccccc1-c1c[nH]c2nc(NC(=O)[C@H]3C[C@H]3C#N)ccc12]

To a solution of cis-2-cyano-N-[3-(2-methoxyphenyl)-1-[[2-(trimethylsilyl)ethoxy]methyl]pyrrolo[2,3-b]pyridin-6-yl]cyclopropane-1-carboxamide (150.0 mg, 0.43 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 55% B to 75% B in 10 min; 254 nm) to afford cis-2-cyano-N-[3-(2-methoxyphenyl)-1H-pyrrolo[2,3-b]pyridin-6-yl]cyclopropane-1-carboxamide (3.7 mg, 3%) as a white solid. LCMS (ESI, m/z): [M+H]⁺=333.1. ¹H NMR (300 MHz, DMSO-d₆): δ 11.66 (s, 1H), 10.93 (s, 1H), 8.01 (d, J=8.4 Hz, 1H), 7.86 (d, J=8.4 Hz, 1H), 7.60 (d, J=2.1 Hz, 1H), 7.55-7.52 (m, 1H), 7.31-7.25 (m, 1H), 7.12 (d, J=7.5 Hz, 1H), 7.05-7.00 (m, 1H), 3.82 (s, 3H), 2.77-2.71 (m, 1H), 2.16-2.10 (m, 1H), 1.63-1.54 (m, 1H), 1.45-1.38 (m, 1H).

### Example S107. Compound 107

### Step 1: Synthesis of trans-2-(bromomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide

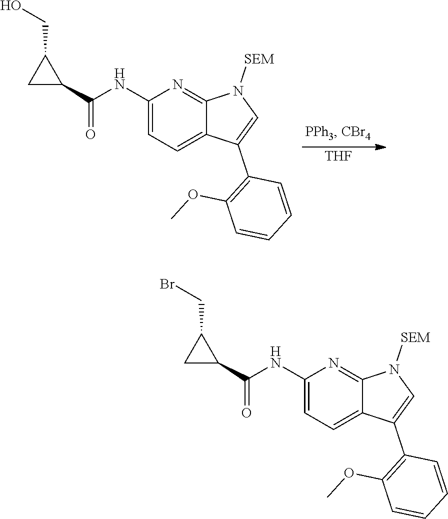 [SMILES: COc1ccccc1-c1cn(COCC[Si](C)(C)C)c2nc(NC(=O)[C@H]3C[C@@H]3CO)ccc12 ; COc1ccccc1-c1cn(COCC[Si](C)(C)C)c2nc(NC(=O)[C@H]3C[C@@H]3CBr)ccc12 ; "THF"]

To a solution of trans-2-(hydroxymethyl)-N-(3-(2-methoxyphenyl)-1-((2-(trimethylsilyl)ethoxy)methyl)-1H-pyrrolo[2,3-b]pyridin-6-yl)cyclopropane-1-carboxamide (300.0 mg, 0.64 mmol) in THF (10.0 mL) was added PPh₃ (185.0 mg, 0.70 mmol) and CBr₄ (319.2 mg, 0.96 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (83/17, v/v) to afford trans-2-(bromomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide (250.0 mg, 40%) as a colorless oil. LCMS (ESI, m/z): [M+H]⁺=530.1.

### Step 2: Synthesis of trans-2-(cyanomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide

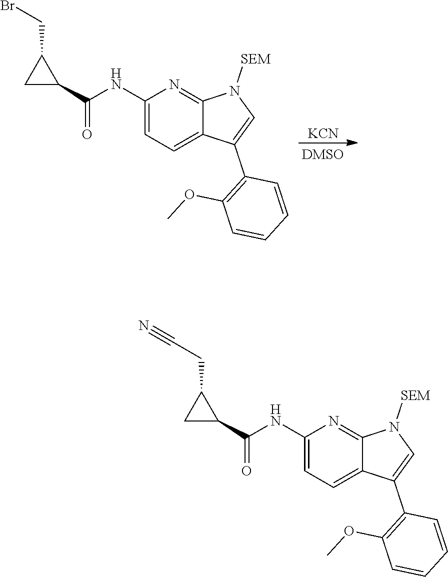

To a solution of trans-2-(bromomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide (200.0 mg, 0.39 mmol) in DMSO (10.0 mL) was added KCN (37.7 mg, 0.58 mmol) at room temperature. The resulting mixture was stirred at 60° C. for 3 h. After the reaction was completed, the resulting mixture was quenched with aq. NaHCO₃ and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (72/28, v/v) to afford trans-2-(cyanomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide (150.0 mg, 81%) as a colorless oil. LCMS (ESI, m/z): [M+H]⁺=477.2.

### Step 3: Synthesis of trans-2-(cyanomethyl)-N-(3-(2-methoxyphenyl)-1H-pyrrolo[2,3-b]pyridin-6-yl)cyclopropane-1-carboxamide (Compound 107)

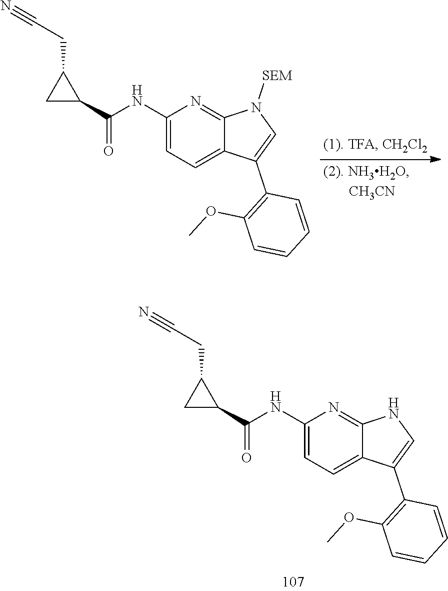 [SMILES: COc1ccccc1-c1cn(COCC[Si](C)(C)C)c2nc(NC(=O)[C@H]3C[C@@H]3CC#N)ccc12 ; COc1ccccc1-c1c[nH]c2nc(NC(=O)[C@H]3C[C@@H]3CC#N)ccc12]

To a solution of trans-2-(cyanomethyl)-N-(3-(2-methoxyphenyl)-1-((2-(trimethylsilyl)ethoxy)methyl)-1H-pyrrolo[2,3-b]pyridin-6-yl)cyclopropane-1-carboxamide (130.0 mg, 0.27 mmol) in CH₂Cl₂ (5.0 mL) was added TFA (5.0 mL) at room temperature. The resulting mixture was stirred at room temperature for 2 h. After the reaction was completed, the resulting mixture was concentrated under reduced pressure. The residue was dissolved in CH₃CN (5.0 mL) and NH₃·H₂O (5.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 42% B to 52% B in 8 min; 254 nm) to afford trans-2-(cyanomethyl)-N-(3-(2-methoxyphenyl)-1H-pyrrolo[2,3-b]pyridin-6-yl)cyclopropane-1-carboxamide (37.2 mg, 39%) as a white solid. LCMS (ESI, m/z): [M+H]⁺=347.2. ¹H NMR (300 MHz, DMSO-d₆): δ 11.58 (s, 1H), 10.70 (s, 1H), 7.98 (d, J=8.7 Hz, 1H), 7.88 (d, J=8.7 Hz, 1H), 7.58-7.52 (m, 2H), 7.30-7.25 (m, 1H), 7.11 (d, J=8.1 Hz, 1H), 7.05-7.00 (m, 1H), 3.82 (s, 3H), 2.73 (d, J=6.6 Hz, 2H), 2.10-2.05 (m, 1H), 1.59-1.52 (m, 1H), 1.14-1.08 (m, 1H), 0.95-0.83 (m, 1H).

### Example S108. Compound 108

### Step 1: Synthesis of trans-methyl 2-((3-(2-methoxyphenyl)-1-((2-(trimethylsilyl)ethoxy)methyl)-1H-pyrrolo[2,3-b]pyridin-6-yl)carbamoyl)cyclopropane-1-carboxylate

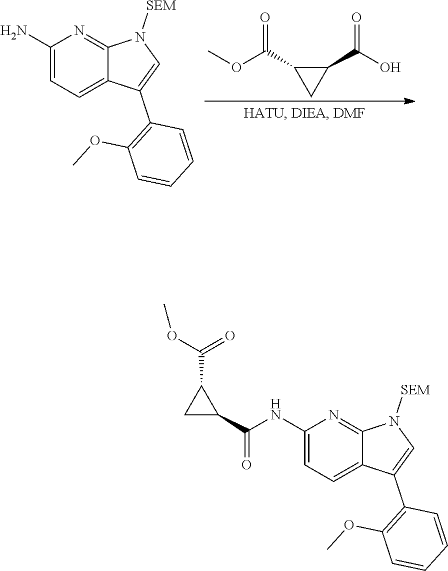

To a solution of 3-(2-methoxyphenyl)-1-[[2-(trimethylsilyl)ethoxy] methyl]pyrrolo[2,3-b] pyridin-6-amine (600.0 mg, 1.62 mmol) in DMF (10.0 mL) was added DIEA (1049.2 mg, 8.12 mmol), trans-2-(methoxycarbonyl) cyclopropane-1-carboxylic acid (280.8 mg, 1.95 mmol) and HATU (926.0 mg, 2.44 mmol) at 0° C. under N₂. The resulting mixture was stirred at 0° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford trans-methyl 2-[[3-(2-methoxyphenyl)-1-[[2-(trimethylsilyl)ethoxy]methyl]pyrrolo[2,3-b]pyridin-6-yl]carbamoyl]cyclopropane-1-carboxylate (660.0 mg, 82%) as a light yellow solid. LCMS (ESI, m/z): [M+H]⁺=496.2.

### Step 2: Synthesis of trans-2-(hydroxylmethyl)-N-(3-(2-methoxyphenyl)-1-((2-(trimethylsilyl)ethoxy)methyl)-1H-pyrrolo[2,3-b] pyridin-6-yl) cyclopropane-1-carboxamide

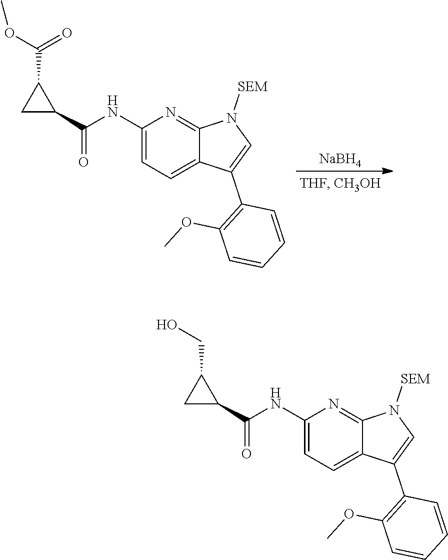 [SMILES: COC(=O)[C@H]1C[C@@H]1C(=O)Nc1ccc2c(-c3ccccc3OC)cn(COCC[Si](C)(C)C)c2n1 ; "NaBH4" ; COc1ccccc1-c1cn(COCC[Si](C)(C)C)c2nc(NC(=O)[C@H]3C[C@@H]3CO)ccc12]

To a solution of trans-methyl 2-[[3-(2-methoxyphenyl)-1-[[2-(trimethylsilyl) ethoxy] methyl] pyrrolo[2,3-b]pyridin-6-yl]carbamoyl]cyclopropane-1-carboxylate (640.0 mg, 1.29 mmol) in THF/CH₃OH (8.0/2.0 mL) was added NaBH₄ (488.5 mg, 12.91 mmol) at room temperature. The resulting mixture was stirred at room temperature for 16 h. After the reaction was completed, the reaction mixture was quenched with CH₃OH and concentrated under reduced pressure. The residue was purified by flash column chromatography with petroleum ether/ethyl acetate (50/50, v/v) to afford trans-2-(hydroxymethyl)-N-[3-(2-methoxyphenyl)-1-[[2-(trimethylsilyl) ethoxy]methyl]pyrrolo[2,3-b] pyridin-6-yl]cyclopropane-1-carboxamide (500.0 mg, 82%) as a white solid. LCMS (ESI, m/z): [M+H]⁺=468.2.

### Step 3: Synthesis of trans-2-(hydroxymethyl)-N-(3-(2-methoxyphenyl)-1H-pyrrolo[2,3-b]pyridin-6-yl)cyclopropane-1-carboxamide (Compound 108)

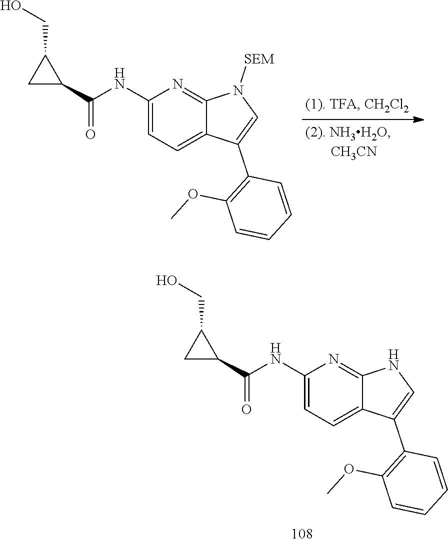 [SMILES: COc1ccccc1-c1cn(COCC[Si](C)(C)C)c2nc(NC(=O)[C@H]3C[C@@H]3CO)ccc12 ; COc1ccccc1-c1c[nH]c2nc(NC(=O)[C@H]3C[C@@H]3CO)ccc12]

To a solution of trans-2-(hydroxymethyl)-N-[3-(2-methoxyphenyl)-1-[[2-(trimethylsilyl)ethoxy] methyl]pyrrolo [2,3-b]pyridin-6-yl]cyclopropane-1-carboxamide (130.0 mg, 0.28 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL). The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 29% B to 59% B in 7 min; 254 nm) to afford trans-2-(hydroxymethyl)-N-[3-(2-methoxyphenyl)-1H-pyrrolo[2,3-b] pyridin-6-yl]cyclopropane-1-carboxamide (20.3 mg, 21%) as a white solid. LCMS (ESI, m/z): [M+H]⁺=338.2. ¹H NMR (300 MHz, DMSO-d₆): δ 11.54 (s, 1H), 10.46 (s, 1H), 7.96 (d, J=8.7 Hz, 1H), 7.87 (d, J=8.7 Hz, 1H), 7.55-7.52 (m, 2H), 7.29-7.23 (m, 1H), 7.10 (d, J=7.5 Hz, 1H), 7.04-6.99 (m, 1H), 4.66-4.62 (m, 1H), 3.81 (s, 3H), 3.49-3.42 (m, 1H), 3.31-3.27 (m, 1H), 1.97-1.93 (m, 1H), 1.49-1.43 (m, 1H), 1.01-0.95 (m, 1H), 0.80-0.75 (m, 1H).

### Example S109. Compound 109

Step 1: Synthesis of tert-butyl N-[2-(4-ethylpiperazin-1-yl)ethyl]carbamate

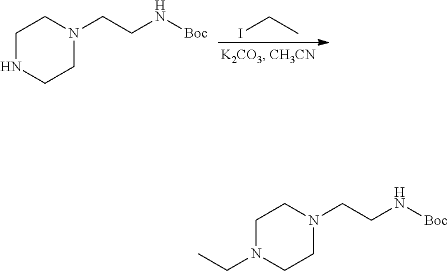

To a solution of tert-butyl N-[2-(piperazin-1-yl)ethyl]carbamate (2.0 g, 8.72 mmol) in CH₃CN (20.0 mL) was added K₂CO₃ (2.4 g, 17.44 mmol) and ethyl iodide (2.0 g, 13.08 mmol). The mixture was stirred at room temperature for 16 h. After the reaction was completed, the resulting mixture was concentrated under vacuum. The residue was purified by flash column chromatography with CH₂Cl₂/MeOH (88/12, v/v) to afford tert-butyl N-[2-(4-ethylpiperazin-1-yl)ethyl]carbamate (400.0 mg, 17%) as a yellow oil. LCMS (ESI, m/z): [M+H]⁺=258.2.

### Step 2: Synthesis of 2-(4-ethylpiperazin-1-yl)ethanamine hydrochloride

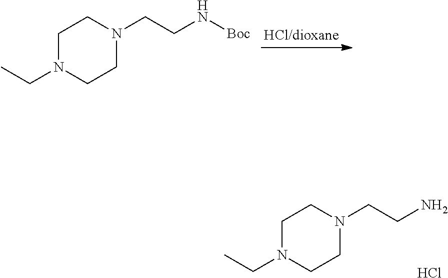 [SMILES: CCN1CCN(CCNC(=O)OC(C)(C)C)CC1 ; CCN1CCN(CCN)CC1 ; Cl]

The solution of tert-butyl N-[2-(4-ethylpiperazin-1-yl)ethyl]carbamate (400.0 mg, 1.55 mmol) in HCl/dioxane (3.0 mL, 4 mol/L) was stirred at room temperature for 2 h. After the reaction was completed, the mixture was filtered. The solid was washed with Et₂O and dried to afford 2-(4-ethylpiperazin-1-yl)ethanamine hydrochloride (200.0 mg, crude) as a yellow solid. LCMS (ESI, m/z): [M+H]⁺=158.2.

### Step 3: Synthesis of 1-[2-(4-ethylpiperazin-1-yl)ethyl]-3-[3-(2-methoxyphenyl)-1-[[2-(trimethylsilyl)ethoxy]methyl]pyrrolo[2,3-b]pyridin-6-yl]urea

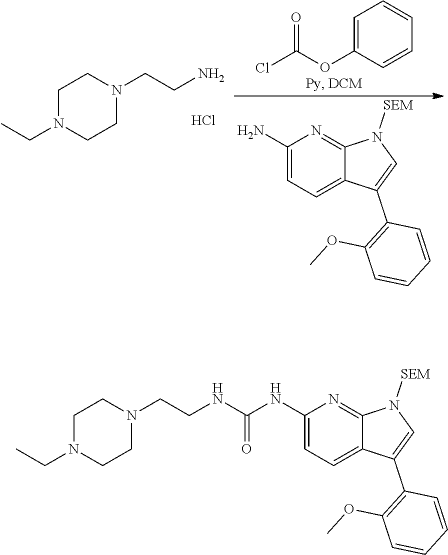

To a solution of 3-(2-methoxyphenyl)-1-[[2-(trimethylsilyl)ethoxy]methyl]pyrrolo[2,3-b]pyridin-6-amine (150.0 mg, 0.41 mmol) in CH₂Cl₂ (3.0 mL) was added pyridine (128.4 mg, 1.62 mmol) and phenyl chloroformate (76.3 mg, 0.49 mmol) at 0° C. The mixture was stirred at room temperature for 16 h. The mixture was concentrated under vacuum. To the above residue was added pyridine (3.0 mL) and 2-(4-ethylpiperazin-1-yl)ethanamine hydrochloride (236.7 mg, crude). The resulting mixture was stirred at 60° C. for another 5 h. After the reaction was completed, the resulting mixture was concentrated under vacuum. The residue was purified by flash column chromatography with petroleum ether/ethyl acetate (5/95, v/v) to afford 1-[2-(4-ethylpiperazin-1-yl)ethyl]-3-[3-(2-methoxyphenyl)-1-[[2-(trimethylsilyl)ethoxy]methyl]pyrrolo[2,3-b]pyridin-6-yl]urea (200.0 mg, 89%) as a yellow solid. LCMS (ESI, m/z): [M+H]⁺=553.3

### Step 4: Synthesis of 1-[2-(4-ethylpiperazin-1-yl)ethyl]-3-[3-(2-methoxyphenyl)-1H-pyrrolo[2,3-b]pyridin-6-yl]urea (Compound 109)

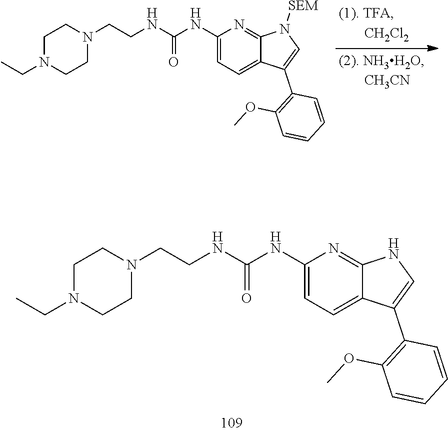 [SMILES: CCN1CCN(CCNC(=O)Nc2ccc3c(-c4ccccc4OC)cn(COCC[Si](C)(C)C)c3n2)CC1 ; CCN1CCN(CCNC(=O)Nc2ccc3c(-c4ccccc4OC)c[nH]c3n2)CC1]

To a solution of 1-[2-(4-ethylpiperazin-1-yl)ethyl]-3-[3-(2-methoxyphenyl)-1-[[2-(trimethylsilyl)ethoxy]methyl]pyrrolo[2,3-b]pyridin-6-yl]urea (180.0 mg, 0.33 mmol) in CH₂Cl₂ (3.0 mL) was added TFA (3.0 mL). The mixture was stirred at room temperature for 2 h. The mixture was concentrated under vacuum. To the above residue was added ACN (3.0 mL) and NH₃·H₂O (3.0 mL). The resulting mixture was stirred at room temperature for another 2 h. After the reaction was completed, the resulting mixture was concentrated under vacuum. The residue was purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 37% B to 47% B in 8 min; 254 nm) to afford 1-[2-(4-ethylpiperazin-1-yl)ethyl]-3-[3-(2-methoxyphenyl)-1H-pyrrolo[2,3-b]pyridin-6-yl]urea (24.6 mg, 17%) as a white solid. LCMS (ESI, m/z): [M+H]⁺=423.2. ¹H NMR (400 MHz, DMSO-d₆): δ 11.42 (s, 1H), 9.18 (s, 1H), 8.30 (s, 1H), 7.90 (d, J=8.4 Hz, 1H), 7.51-7.49 (m, 1H), 7.45 (d, J=2.0 Hz, 1H), 7.28-7.23 (m, 1H), 7.10 (d, J=8.4 Hz, 2H), 7.03-6.99 (m, 1H), 3.81 (s, 3H), 2.47-2.44 (m, 6H), 2.36-2.26 (m, 6H), 0.99-0.96 (m, 3H).

### Example S110. Compound 110

### Step 1: Synthesis of tert-butyl N-[2-[1-(oxetan-3-yl)piperidin-4-yl]ethyl]carbamate

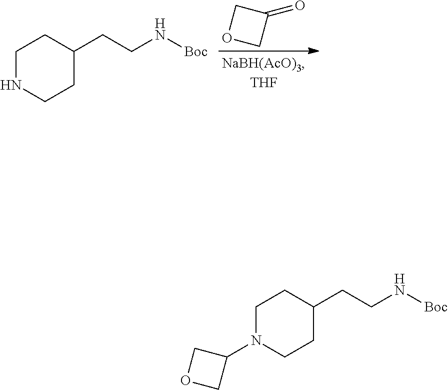

To a solution of tert-butyl N-[2-(piperidin-4-yl)ethyl]carbamate (1.0 g, 4.38 mmol) in THE (50.0 mL) was added 3-oxetanone (0.4 g, 5.26 mmol) and NaBH(OAc)₃ (1.4 g, 6.57 mmol at room temperature. The resulting mixture was stirred at room temperature for 16 h. After the reaction was completed, the resulting mixture was concentrated under reduced pressure. The residue was purified by flash column chromatography with CH₂Cl₂/MeOH (10/1, v/v) to afford tert-butyl N-[2-[1-(oxetan-3-yl)piperidin-4-yl]ethyl]carbamate (900.0 mg, 72%) as a brown solid. LCMS (ESI, m/z): [M+H]⁺=285.0.

### Step 2: Synthesis of 2-[1-(oxetan-3-yl)piperidin-4-yl]ethanamine

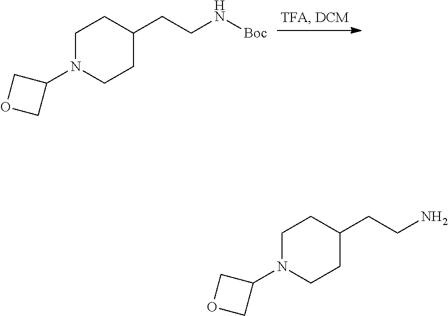 [SMILES: CC(C)(C)OC(=O)NCCC1CCN(C2COC2)CC1 ; NCCC1CCN(C2COC2)CC1]

To a solution of tert-butyl N-[2-[1-(oxetan-3-yl)piperidin-4-yl]ethyl]carbamate (800.0 mg, 2.81 mmol) in DCM (6.0 mL) was added TFA (6.0 mL) at room temperature. The resulting mixture was stirred at room temperature for 2 h. After the reaction was completed, the mixture was basified to pH=8 with saturated NaHCO₃ (aq.). The mixture was diluted with H₂O and extracted with ethyl acetate. The combined organic layer was washed with brine, dried over anhydrous sodium sulfate and filtered. The filtrate was concentrated under reduced pressure to afford 2-[1-(oxetan-3-yl)piperidin-4-yl]ethanamine (500.0 mg, crude) as a brown oil. LCMS (ESI, m/z): [M+H]⁺=185.2.

### Step 3: Synthesis of 3-[3-(2-methoxyphenyl)-1-[[2-(trimethylsilyl)ethoxy]methyl]pyrrolo[2,3-b]pyridin-6-yl]-1-[2-[4-(oxetan-3-yl)piperazin-1-yl]ethyl]urea

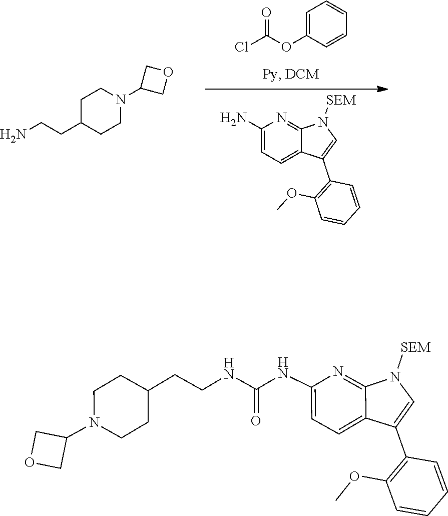 [SMILES: O=C(Cl)Oc1ccccc1 ; NCCC1CCN(C2COC2)CC1 ; COc1ccccc1-c1cn(COCC[Si](C)(C)C)c2nc(N)ccc12 ; COc1ccccc1-c1cn(COCC[Si](C)(C)C)c2nc(NC(=O)NCCC3CCN(C4COC4)CC3)ccc12]

To a solution of 3-(2-methoxyphenyl)-1-[[2-(trimethylsilyl)ethoxy]methyl]pyrrolo[2,3-b]pyridin-6-amine (130.0 mg, 0.35 mmol) in DCM (5.0 mL) was added pyridine (112.7 mg, 1.41 mmol) and phenyl chloroformate (66.1 mg, 0.42 mmol) at room temperature. The resulting mixture was stirred at room temperature for 16 h. The resulting mixture was concentrated under reduced pressure. To the above residue was added pyridine (6.0 mL) and 2-[1-(oxetan-3-yl)piperidin-4-yl]ethanamine (129.7 mg, 0.70 mmol) at room temperature. The resulting mixture was stirred at 60° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford 3-[3-(2-methoxyphenyl)-1-[[2-(trimethylsilyl)ethoxy]methyl]pyrrolo[2,3-b]pyridin-6-yl]-1-[2-[4-(oxetan-3-yl)piperazin-1-yl]ethyl]urea (100.0 mg, 49%) as a yellow oil. LCMS (ESI, m/z): [M+H]⁺=581.3.

### Step 4: Synthesis of 3-[3-(2-methoxyphenyl)-1H-pyrrolo[2,3-b]pyridin-6-yl]-1-[2-[1-(oxetan-3-yl)piperidin-4-yl]ethyl]urea (Compound 110)

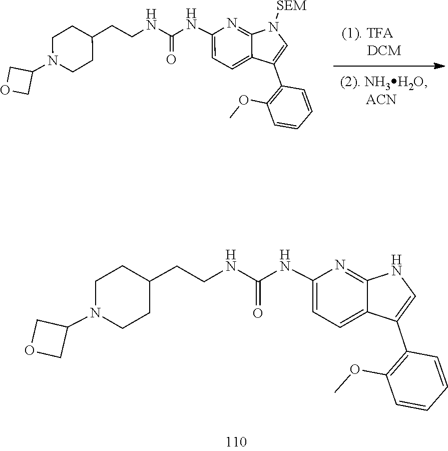 [SMILES: COc1ccccc1-c1cn(COCC[Si](C)(C)C)c2nc(NC(=O)NCCC3CCN(C4COC4)CC3)ccc12 ; COc1ccccc1-c1c[nH]c2nc(NC(=O)NCCC3CCN(C4COC4)CC3)ccc12]

To a solution of 3-[3-(2-methoxyphenyl)-1-[[2-(trimethylsilyl)ethoxy]methyl]pyrrolo[2,3-b]pyridin-6-yl]-1-[2-[4-(oxetan-3-yl)piperazin-1-yl]ethyl]urea (90.0 mg, 0.16 mmol) in CH₂Cl₂ (4.0 mL) was added TFA (4.0 mL) at room temperature. The resulting mixture was stirred at room temperature for 16 h. The resulting mixture was concentrated under reduced pressure. The residue was dissolved in CH₃CN (4.0 mL) and NH₃·H₂O (4.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 9 min; 254 nm) to afford 3-[3-(2-methoxyphenyl)-1H-pyrrolo[2,3-b]pyridin-6-yl]-1-[2-[1-(oxetan-3-yl)piperidin-4-yl]ethyl]urea (2.2 mg, 3%) as a light yellow solid. LCMS (ESI, m/z): [M+H]⁺=450.3. ¹H NMR (400 MHz, DMSO-d₆): δ 11.49 (s, 1H), 9.13 (s, 1H), 8.55 (s, 1H), 7.89 (d, J=8.4 Hz, 1H), 7.53 (s, 1H), 7.45 (d, J=2.4 Hz, 1H), 7.26-7.22 (m, 1H), 7.11-7.08 (m, 1H), 7.02-6.95 (m, 2H), 4.52-4.49 (m, 2H), 4.42-4.39 (m, 2H), 3.80 (s, 3H), 3.30-3.25 (m, 2H), 2.68-2.64 (m, 2H), 1.79-1.69 (m, 4H), 1.49-1.44 (m, 2H), 1.37-1.28 (m, 1H), 1.24-1.17 (m, 2H).

### Example S111. Compound 111

### Step 1: Synthesis of (E)-N′-(5-bromo-4-methoxypyridin-2-yl)-N,N-dimethylformimidamide

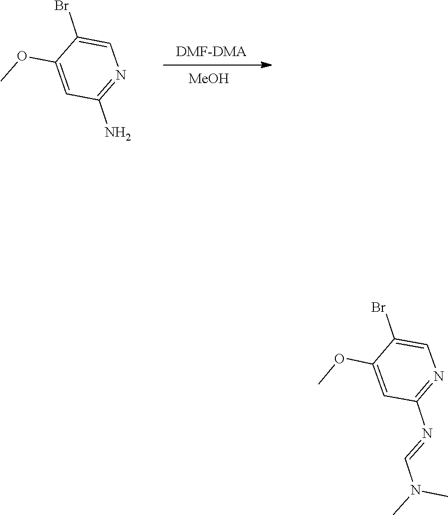

To a solution of 5-bromo-4-methoxypyridin-2-amine (1.0 g, 4.93 mmol) in MeOH (10.0 mL) was added DMF-DMA (704.2 mg, 5.91 mmol) at room temperature. The resulting mixture was stirred at 75° C. for 3 h. After the reaction was completed, the resulting mixture concentrated under reduced pressure to afford (E)-N′-(5-bromo-4-methoxypyridin-2-yl)-N,N-dimethylformimidamide (870.0 mg, crude) as a yellow solid. LCMS (ESI, m/z): [M+H]⁺=258.0.

### Step 2: Synthesis of 6-bromo-7-methoxy-[1,2,4]triazolo[1,5-a]pyridine

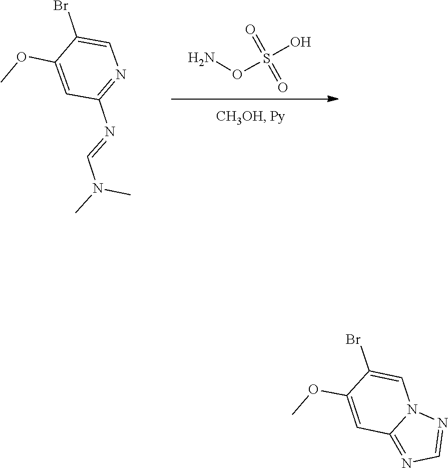

To a solution of (E)-N′-(5-bromo-4-methoxypyridin-2-yl)-N,N-dimethylformimidamide (550.0 mg, 2.13 mmol) in CH₃OH/Pyridine (5.0/0.5 mL) was added (aminooxy)sulfonic acid (361.5 mg, 3.20 mmol) at 0° C. under N₂. The resulting mixture was stirred at room temperature for 16 h. After the reaction was completed, the resulting mixture was quenched with aq. NaHCO₃ and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (1/1, v/v) to afford 6-bromo-7-methoxy-[1,2,4]triazolo[1,5-a]pyridine (100.0 mg, 20%) as a light yellow solid. LCMS (ESI, m/z): [M+H]⁺=228.0.

### Step 3: Synthesis of 6-(6-chloro-1-((2-(trimethylsilyl)ethoxy)methyl)-1H-pyrrolo[2,3-b]pyridin-3-yl)-7-methoxy-[1,2,4]triazolo[1,5-a]pyridine

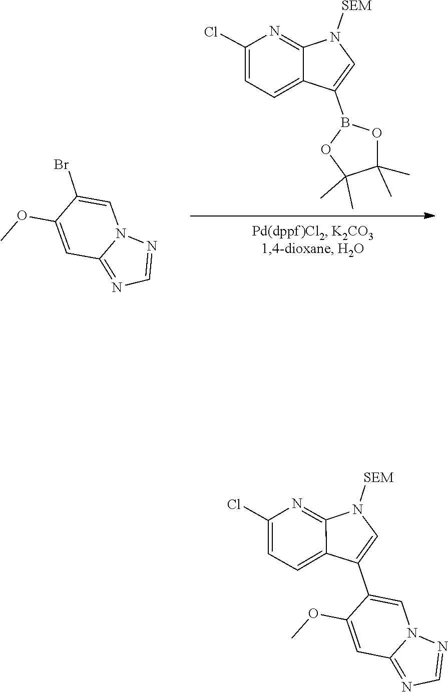 [SMILES: COc1cc2ncnn2cc1Br ; CC1(C)OB(c2cn(COCC[Si](C)(C)C)c3nc(Cl)ccc23)OC1(C)C ; COc1cc2ncnn2cc1-c1cn(COCC[Si](C)(C)C)c2nc(Cl)ccc12]

To a solution of 6-bromo-7-methoxy-[1,2,4]triazolo[1,5-a]pyridine (150.0 mg, 0.66 mmol) in 1,4-dioxane/H₂O (5.0/1.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (268.9 mg, 0.66 mmol), K₂CO₃ (272.7 mg, 1.97 mmol) and Pd(dppf)Cl₂ (48.1 mg, 0.07 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/90, v/v) to afford 6-(6-chloro-1-((2-(trimethylsilyl)ethoxy)methyl)-1H-pyrrolo[2,3-b]pyridin-3-yl)-7-methoxy-[1,2,4]triazolo[1,5-a]pyridine (100.0 mg, 35%) as a light brown solid. LCMS (ESI, m/z): [M+H]⁺=430.1.

### Step 4: Synthesis of (1S,2S)-2-fluoro-N-(3-(7-methoxy-[1,2,4]triazolo[1,5-a]pyridin-6-yl)-1-((2-(trimethylsilyl)ethoxy)methyl)-1H-pyrrolo[2,3-b]pyridin-6-yl)cyclopropane-1-carboxamide

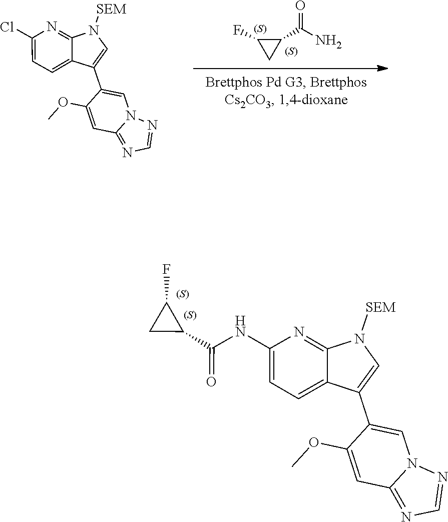

To a solution of 6-(6-chloro-1-((2-(trimethylsilyl)ethoxy)methyl)-1H-pyrrolo[2,3-b]pyridin-3-yl)-7-methoxy-[1,2,4]triazolo[1,5-a]pyridine (80.0 mg, 0.19 mmol) in 1,4-dioxane (4.0 mL) was added (1S,2S)-2-fluorocyclopropane-1-carboxamide (95.9 mg, 0.93 mmol), Cs₂CO₃ (181.9 mg, 0.56 mmol), BrettPhos (20.0 mg, 0.04 mmol) and BrettPhos Pd G3 (16.9 mg, 0.02 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (30/70, v/v) to afford (1S,2S)-2-fluoro-N-(3-(7-methoxy-[1,2,4]triazolo[1,5-a]pyridin-6-yl)-1-((2-(trimethylsilyl)ethoxy)methyl)-1H-pyrrolo[2,3-b]pyridin-6-yl)cyclopropane-1-carboxamide (70.0 mg, 76%) as a yellow solid. LCMS (ESI, m/z): [M+H]⁺=497.2.

### Step 5: Synthesis of (1S,2S)-2-fluoro-N-(3-(7-methoxy-[1,2,4]triazolo[1,5-a]pyridin-6-yl)-1H-pyrrolo[2,3-b]pyridin-6-yl)cyclopropane-1-carboxamide (Compound 111)

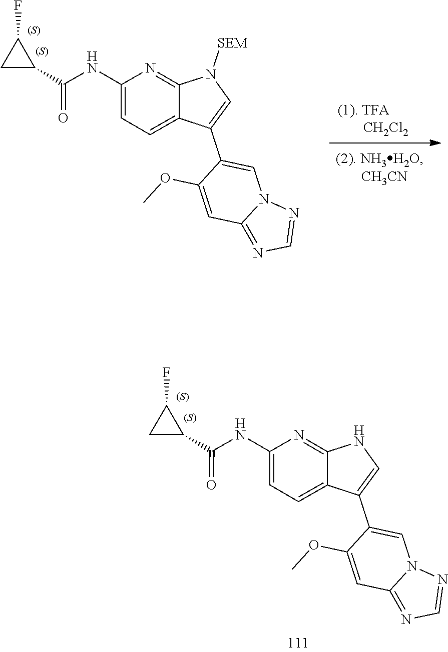

To a solution of (1S,2S)-2-fluoro-N-(3-(7-methoxy-[1,2,4]triazolo[1,5-a]pyridin-6-yl)-1-((2-(trimethylsilyl)ethoxy)methyl)-1H-pyrrolo[2,3-b]pyridin-6-yl)cyclopropane-1-carboxamide (70.0 mg, 0.14 mmol) in CH₂Cl₂ (3.0 mL) was added TFA (3.0 mL) at room temperature. The resulting mixture was stirred at room temperature for 2 h. After the reaction was completed, the resulting mixture was concentrated under reduced pressure. The residue was dissolved in CH₃CN (3.0 mL) and NH₃·H₂O (3.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 23% B to 33% B in 8 min; 254 nm) to afford (1S,2S)-2-fluoro-N-(3-(7-methoxy-[1,2,4]triazolo[1,5-a]pyridin-6-yl)-1H-pyrrolo[2,3-b]pyridin-6-yl)cyclopropane-1-carboxamide (14.3 mg, 27%) as a white solid. LCMS (ESI, m/z): [M+H]⁺=367.2. ¹H NMR (300 MHz, DMSO-d₆): δ 11.77 (s, 1H), 10.68 (s, 1H), 8.90 (s, 1H), 8.35 (s, 1H), 8.02-7.91 (m, 2H), 7.68 (d, J=2.4 Hz, 1H), 7.36 (s, 1H), 5.08-4.72 (m, 1H), 3.96 (s, 3H), 2.30-2.13 (m, 1H), 1.78-1.55 (m, 1H), 1.23-1.04 (m, 1H).

### Example S112. Compound 112

### Step 1: Synthesis of 3-(6-chloro-1-[[2-(trimethylsilyl)ethoxy]methyl]pyrazolo[3,4-b]pyridin-3-yl)-2-methoxy-4-methylpyridine

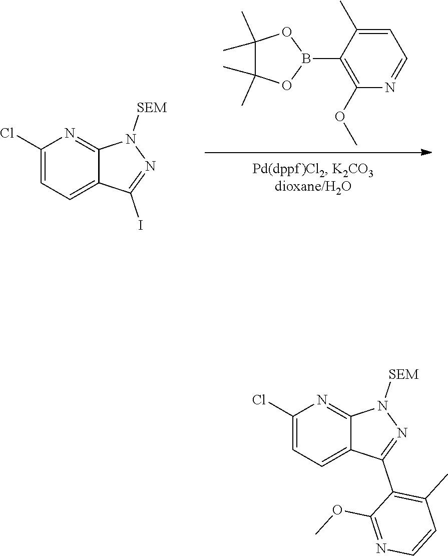 [SMILES: C[Si](C)(C)CCOCn1nc(I)c2ccc(Cl)nc21 ; COc1nccc(C)c1B1OC(C)(C)C(C)(C)O1 ; COc1nccc(C)c1-c1nn(COCC[Si](C)(C)C)c2nc(Cl)ccc12]

To a solution of 6-chloro-3-iodo-1-[[2-(trimethylsilyl)ethoxy]methyl]pyrazolo[3,4-b]pyridine (400.0 mg, 0.98 mmol) in dioxane/H₂O (10.0 mL/2.0 mL) was added 2-methoxy-4-methyl-3-(4,4,5,5-tetramethyl-1,3,2-dioxaborolan-2-yl)pyridine (243.2 mg, 0.98 mmol), K₂CO₃ (404.8 mg, 2.93 mmol) and Pd(dppf)Cl₂ (71.4 mg, 0.10 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-(6-chloro-1-[[2-(trimethylsilyl)ethoxy]methyl]pyrazolo[3,4-b]pyridin-3-yl)-2-methoxy-4-methylpyridine (100.0 mg, 25%) as a colorless oil. LCMS (ESI, m/z): [M+H]⁺=405.1.

### Step 2: Synthesis of (1S,2S)-2-fluoro-N-[3-(2-methoxy-4-methylpyridin-3-yl)-1-[[2-(trimethylsilyl)ethoxy]methyl]pyrazolo[3,4-b]pyridin-6-yl]cyclopropane-1-carboxamide

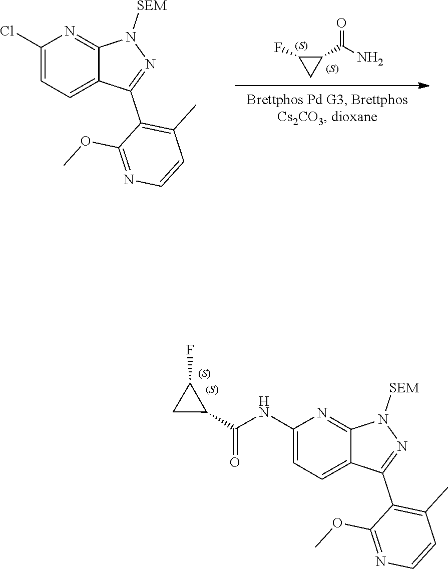

To a solution of 3-(6-chloro-1-[[2-(trimethylsilyl)ethoxy]methyl]pyrazolo[3,4-b]pyridin-3-yl)-2-methoxy-4-methylpyridine (160.0 mg, 0.40 mmol) in 1,4-dioxane (10.0 mL) was added (1S,2S)-2-fluorocyclopropane-1-carboxamide (203.7 mg, 1.98 mmol), BrettPhos (42.4 mg, 0.08 mmol), Cs₂CO₃ (386.2 mg, 1.19 mmol) and BrettPhos Pd G3 (35.8 mg, 0.04 mmol) at room temperature under N₂. The reaction mixture was stirred with microwave at 120° C. for 1.5 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-[3-(2-methoxy-4-methylpyridin-3-yl)-1-[[2-(trimethylsilyl)ethoxy]methyl]pyrazolo[3,4-b]pyridin-6-yl]cyclopropane-1-carboxamide (57.0 mg, 31%) as a yellow oil. LCMS (ESI, m/z): [M+H]⁺=472.2.

### Step 3: Synthesis of (1S,2S)-2-fluoro-N-[3-(2-methoxy-4-methylpyridin-3-yl)-1H-pyrazolo[3,4-b]pyridin-6-yl]cyclopropane-1-carboxamide (Compound 112)

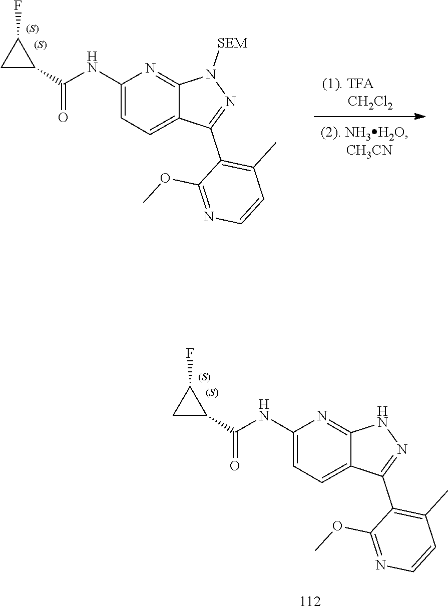

To a solution of (1S,2S)-2-fluoro-N-[3-(2-methoxy-4-methylpyridin-3-yl)-1-[[2-(trimethylsilyl)ethoxy]methyl]pyrazolo[3,4-b]pyridin-6-yl]cyclopropane-1-carboxamide (57.0 mg, 0.12 mmol) in CH₂Cl₂ (3.0 mL) was added TFA (3.0 mL) at room temperature. The resulting mixture was stirred at room temperature for 4 h. The resulting mixture was concentrated under reduced pressure. The residue was dissolved in CH₃CN (3.0 mL) and NH₃·H₂O (3.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 33% B to 38% B in 8 min; 254 nm) to afford (1S,2S)-2-fluoro-N-[3-(2-methoxy-4-methylpyridin-3-yl)-1H-pyrazolo[3,4-b]pyridin-6-yl]cyclopropane-1-carboxamide (10.9 mg, 26%) as a white solid. LCMS (ESI, m/z): [M+H]⁺=342.1. ¹H NMR (400 MHz, DMSO-d₆): δ 13.53 (s, 1H), 10.99 (s, 1H), 8.13 (s, 1H), 7.98-7.90 (m, 2H), 7.02 (s, 1H), 5.03-4.87 (m, 1H), 3.79 (s, 3H), 2.27-2.20 (m, 4H), 1.70-1.64 (m, 1H), 1.30-1.15 (m, 1H).

### Example S113. Compound 113

### Step 1: Synthesis of 3-(6-chloro-1-[[2-(trimethylsilyl)ethoxy]methyl]pyrrolo[2,3-b]pyridin-3-yl)-2-methoxy-4-methylpyridine

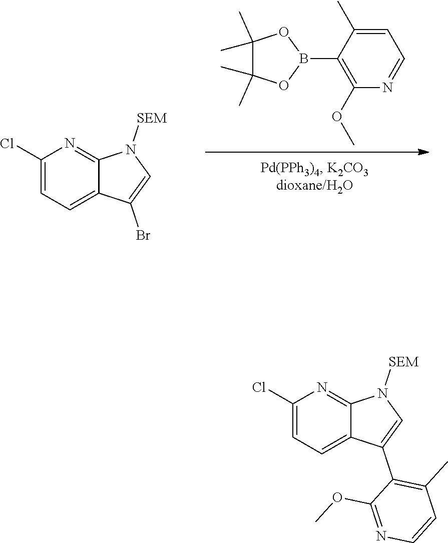 [SMILES: C[Si](C)(C)CCOCn1cc(Br)c2ccc(Cl)nc21 ; COc1nccc(C)c1B1OC(C)(C)C(C)(C)O1 ; COc1nccc(C)c1-c1cn(COCC[Si](C)(C)C)c2nc(Cl)ccc12]

To a solution of 3-bromo-6-chloro-1-[[2-(trimethylsilyl)ethoxy]methyl]pyrrolo[2,3-b]pyridine (500.0 mg, 1.38 mmol) in 1,4-dioxane/H₂O (25.0 mL/5.0 mL) was added 2-methoxy-4-methyl-3-(4,4,5,5-tetramethyl-1,3,2-dioxaborolan-2-yl)pyridine (413.2 mg, 1.66 mmol), K₂CO₃ (382.1 mg, 2.76 mmol) and Pd(PPh₃)₄ (159.7 mg, 0.14 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford 3-(6-chloro-1-[[2-(trimethylsilyl)ethoxy]methyl]pyrrolo[2,3-b]pyridin-3-yl)-2-methoxy-4-methylpyridine (160.0 mg, 28%) as a colorless oil. LCMS (ESI, m/z): [M+H]⁺=404.1.

### Step 2: Synthesis of (1S,2S)-2-fluoro-N-[3-(2-methoxy-4-methylpyridin-3-yl)-1-[[2-(trimethylsilyl)ethoxy]methyl]pyrrolo[2,3-b]pyridin-6-yl]cyclopropane-1-carboxamide

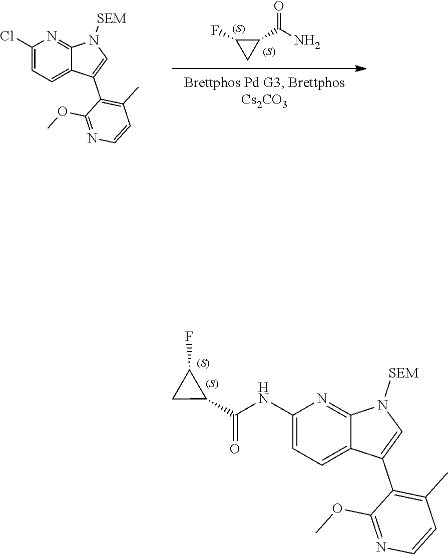

To a solution of 3-(6-chloro-1-[[2-(trimethylsilyl)ethoxy]methyl]pyrrolo[2,3-b]pyridin-3-yl)-2-methoxy-4-methylpyridine (140.0 mg, 0.35 mmol) in 1,4-dioxane (5.0 mL) was added (1S,2S)-2-fluorocyclopropane-1-carboxamide (178.6 mg, 1.73 mmol), BrettPhos (37.2 mg, 0.07 mmol), Cs₂CO₃ (338.7 mg, 1.04 mmol) and BrettPhos Pd G3 (31.4 mg, 0.04 mmol) at room temperature under N₂. The reaction mixture was stirred with microwave at 120° C. for 1.5 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford (1S,2S)-2-fluoro-N-[3-(2-methoxy-4-methylpyridin-3-yl)-1-[[2-(trimethylsilyl)ethoxy]methyl]pyrrolo[2,3-b]pyridin-6-yl]cyclopropane-1-carboxamide (80.0 mg, 49%) as a light yellow oil. LCMS (ESI, m/z): [M+H]⁺=471.2.

### Step 3: Synthesis of (1S,2S)-2-fluoro-N-[3-(2-methoxy-4-methylpyridin-3-yl)-1H-pyrrolo[2,3-b]pyridin-6-yl]cyclopropane-1-carboxamide (Compound 113)

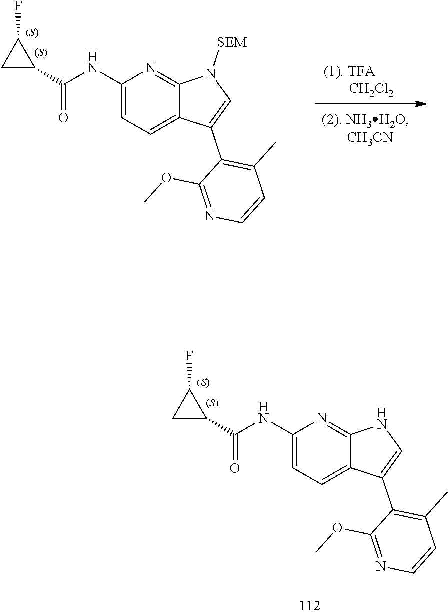

To a solution of (1S,2S)-2-fluoro-N-[3-(2-methoxy-4-methylpyridin-3-yl)-1-[[2-(trimethylsilyl)ethoxy]methyl]pyrrolo[2,3-b]pyridin-6-yl]cyclopropane-1-carboxamide (80.0 mg, 0.17 mmol) in CH₂Cl₂ (3.0 mL) was added TFA (3.0 mL) at room temperature. The resulting mixture was stirred at room temperature for 16 h. The resulting mixture was concentrated under reduced pressure. The residue was dissolved in CH₃CN (3.0 mL) and NH₃·H₂O (3.0 mL). The resulting mixture was stirred at room temperature for another 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 34% B to 44% B in 8 min; 254 nm) to afford (1S,2S)-2-fluoro-N-[3-(2-methoxy-4-methylpyridin-3-yl)-1H-pyrrolo[2,3-b]pyridin-6-yl]cyclopropane-1-carboxamide (6.9 mg, 11%) as a white solid. LCMS (ESI, m/z): [M+H]⁺=341.0. ¹H NMR (400 MHz, DMSO-d₆): δ 11.59 (s, 1H), 10.62 (s, 1H), 8.02 (d, J=4.8 Hz, 1H), 7.84 (d, J=8.8 Hz, 1H), 7.55 (d, J=8.4 Hz, 1H), 7.38 (d, J=2.4 Hz, 1H), 6.97 (d, J=5.2 Hz, 1H), 5.02-4.81 (m, 1H), 3.75 (s, 3H), 2.23-2.18 (m, 4H), 1.68-1.61 (m, 1H), 1.19-1.14 (m, 1H).

### Example S114. Compound 114

### Step 1: Synthesis of 4-bromo-2-nitropyridin-3-ol

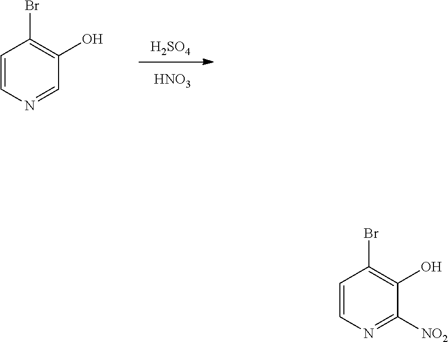

To a solution of 4-bromopyridin-3-ol (5.0 g, 28.74 mmol) in sulfuric acid (25.0 mL) was added nitric acid (5.4 g, 86.21 mmol) at room temperature under N₂. The resulting mixture was stirred at room temperature for 3 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/1, v/v) to afford 4-bromo-2-nitropyridin-3-ol (2.3 g, 19%) as a yellow solid. LCMS (ESI, m/z): [M+H]⁺=218.9.

### Step 2: Synthesis of 4-bromo-3-methoxy-2-nitropyridine

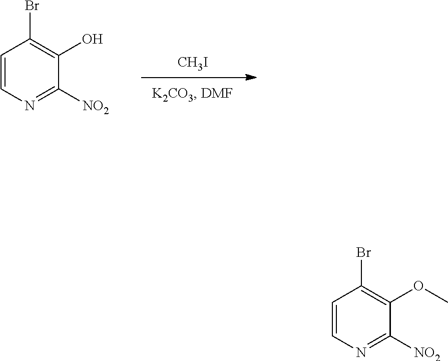

To a solution of 4-bromo-2-nitropyridin-3-ol (2.3 g, 10.50 mmol) in DMF (115.0 mL) was added K₂CO₃ (2.9 g, 21.01 mmol) at room temperature. The resulting mixture was stirred at room temperature for 10 min. Then CH₃I (3.0 g, 21.11 mmol) was added to the mixture at room temperature. The resulting mixture was stirred at room temperature for another 16 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/1, v/v) to afford 4-bromo-3-methoxy-2-nitropyridine (1.8 g, 73%) as a white solid LCMS (ESI, m/z): [M+H]⁺=232.9.

### Step 3: Synthesis of 4-bromo-3-methoxypyridin-2-amine

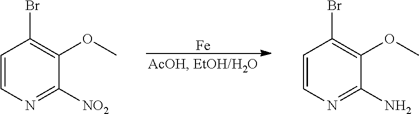

To a solution of 4-bromo-3-methoxy-2-nitropyridine (1.8 g, 7.73 mmol) in AcOH (10.0 mL)/EtOH (10.0 mL)/H₂O (5.0 mL) was added Fe (2.2 g, 38.68 mmol) at room temperature. The resulting mixture was stirred at room temperature for 1 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography petroleum ether/ethyl acetate (1/1, v/v) to afford 4-bromo-3-methoxypyridin-2-amine (1.3 g, 83%) as a white solid. LCMS (ESI, m/z): [M+H]⁺=203.1.

### Step 4: Synthesis of 7-bromo-8-methoxyimidazo[1,2-a]pyridine

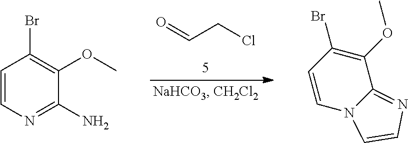

To a solution of 4-bromo-3-methoxypyridin-2-amine (650.0 mg, 3.20 mmol) in sat NaHCO₃ (25.0 mL)/DCM (25.0 mL) was added chloroacetaldehyde (1.6 g, 8.00 mmol) at room temperature. The resulting mixture was stirred at room temperature for 16 h. After the reaction was completed, the reaction mixture was diluted with water and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 7-bromo-8-methoxyimidazo[1,2-a]pyridine (450.0 mg, 62%) as a white solid. LCMS (ESI, m/z): [M+H]⁺=227.0.

### Step 5: Synthesis of 6-chloro-3-[8-methoxyimidazo[1,2-a]pyridin-7-yl]-1-[[2-(trimethylsilyl)ethoxy]methyl]pyrrolo[2,3-b]pyridine

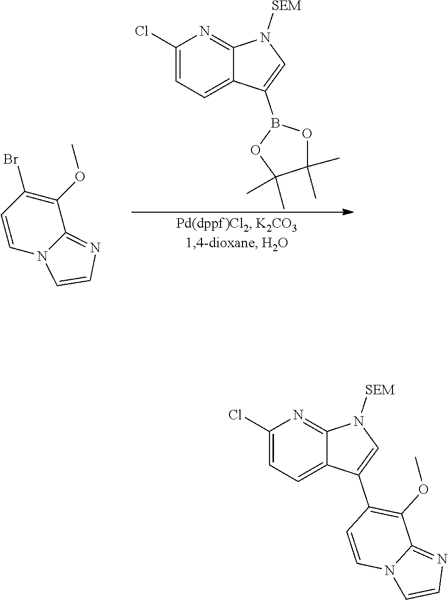 [SMILES: COc1c(Br)ccn2ccnc12 ; CC1(C)OB(c2cn(COCC[Si](C)(C)C)c3nc(Cl)ccc23)OC1(C)C ; COc1c(-c2cn(COCC[Si](C)(C)C)c3nc(Cl)ccc23)ccn2ccnc12]

To a solution of 7-bromo-8-methoxyimidazo[1,2-a]pyridine (500.0 mg, 2.20 mmol) in dioxane/H₂O (20.0 mL/4.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (900.2 mg, 2.20 mmol), K₂CO₃ (913.0 mg, 6.61 mmol), Pd(dppf)Cl₂ (161.1 mg, 0.22 mmol) at room temperature under N₂. The resulting mixture was stirred at 80° C. for 16 h under N₂. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-chloro-3-[8-methoxyimidazo[1,2-a]pyridin-7-yl]-1-[[2-(trimethylsilyl)ethoxy]methyl]pyrrolo[2,3-b]pyridine (140.0 mg, 15%) as a colorless oil. LCMS (ESI, m/z): [M+H]⁺=429.1.

### Step 6: Synthesis of (1S,2S)-2-fluoro-N-(3-[8-methoxyimidazo[1,2-a]pyridin-7-yl]-1-[[2-(trimethylsilyl)ethoxy]methyl]pyrrolo[2,3-b]pyridin-6-yl)cyclopropane-1-carboxamide

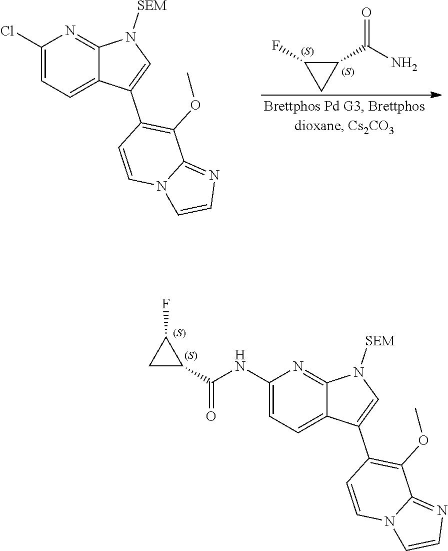

To a solution of 6-chloro-3-[8-methoxyimidazo[1,2-a]pyridin-7-yl]-1-[[2-(trimethylsilyl)ethoxy]methyl]pyrrolo[2,3-b]pyridine (120.0 mg, 0.28 mmol) in 1,4-dioxane (10.0 mL) was added (1S,2S)-2-fluorocyclopropane-1-carboxamide (144.2 mg, 1.40 mmol), BrettPhos (30.0 mg, 0.06 mmol), Cs₂CO₃ (273.4 mg, 0.84 mmol) and BrettPhos Pd G3 (25.4 mg, 0.03 mmol) at room temperature under N₂. The resulting mixture was stirred with microwave at 120° C. for 90 min. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford (1S,2S)-2-fluoro-N-(3-[8-methoxyimidazo[1,2-a]pyridin-7-yl]-1-[[2-(trimethylsilyl)ethoxy]methyl]pyrrolo[2,3-b]pyridin-6-yl)cyclopropane-1-carboxamide (70.0 mg, 50%) as a yellow oil. LCMS (ESI, m/z): [M+H]⁺=496.2.

### Step 7: Synthesis of (1S,2S)-2-fluoro-N-(3-[8-methoxyimidazo[1,2-a]pyridin-7-yl]-1H-pyrrolo[2,3-b]pyridin-6-yl)cyclopropane-1-carboxamide (Compound 114)

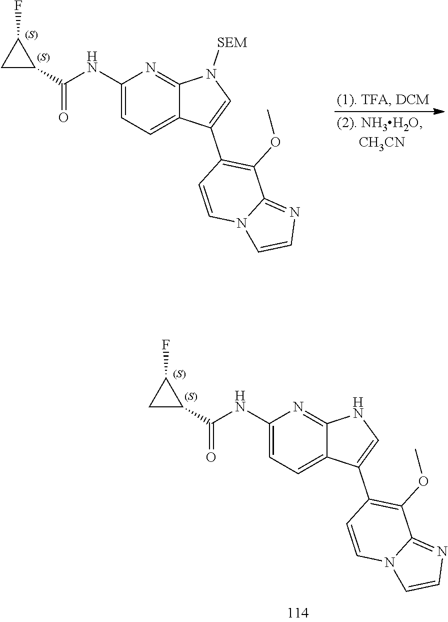

To a solution of (1S,2S)-2-fluoro-N-(3-[8-methoxyimidazo[1,2-a]pyridin-7-yl]-1-[[2-(trimethylsilyl)ethoxy]methyl]pyrrolo[2,3-b]pyridin-6-yl)cyclopropane-1-carboxamide (70.0 mg, 0.14 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in CH₃CN (5.0 mL) was added NH₃·H₂O (5.0 mL) at room temperature. The resulting mixture was stirred at room temperature for another 2 h. After the reaction was completed, the resulting mixture was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 26% B to 35% B in 8 min; 254 nm) to afford (1S,2S)-2-fluoro-N-(3-[8-methoxyimidazo[1,2-a]pyridin-7-yl]-1H-pyrrolo[2,3-b]pyridin-6-yl)cyclopropane-1-carboxamide (11.7 mg, 23%) as a white solid. LCMS (ESI, m/z): [M+H]⁺=366.2. ¹H NMR (400 MHz, DMSO-d₆): δ 11.78 (s, 1H), 10.67 (s, 1H), 8.35 (d, J=6.4 Hz, 1H), 8.14 (d, J=8.4 Hz, 1H), 7.98-7.92 (m, 2H), 7.74 (s, 1H), 7.56 (s, 1H), 7.15 (d, J=6.4 Hz, 1H), 5.02-4.85 (m, 1H), 4.12 (s, 3H), 2.25-2.20 (m, 1H), 1.69-1.64 (m, 1H), 1.22-1.10 (m, 1H).

### Example S115. Compound 115

### Step 1: Synthesis of 4-bromo-2-nitropyridin-3-ol

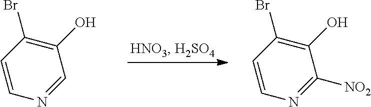 [SMILES: Oc1cnccc1Br ; O=[N+]([O-])c1nccc(Br)c1O]

To a solution of 4-bromopyridin-3-ol (10.0 g, 57.47 mmol) in H₂SO₄ (30.0 mL) was added HNO₃ (10.0 mL) at 0° C. under N₂. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 4-bromo-2-nitropyridin-3-ol (6.3 g, 35%) as an off-white solid. LCMS (ESI, m/z): [M+H]⁺=218.9.

### Step 2: Synthesis of 4-bromo-3-methoxy-2-nitropyridine

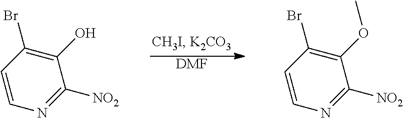

To a solution of 4-bromo-2-nitropyridin-3-ol (6.3 g, 28.77 mmol) in DMF (20.0 mL) was added K₂CO₃ (7.9 g, 56.94 mmol) and CH₃I (4.0 g, 28.77 mmol) at room temperature under N₂. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (93/7, v/v) to afford 4-bromo-3-methoxy-2-nitropyridine (3.2 g, 48%) as a white solid. LCMS (ESI, m/z): [M+H]⁺=232.9.

### Step 3: Synthesis of 4-bromo-3-methoxypyridin-2-amine

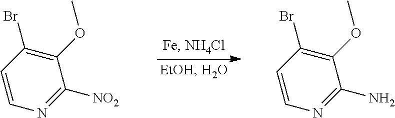

To a solution of 4-bromo-3-methoxy-2-nitropyridine (3.2 g, 13.73 mmol) in EtOH/H₂O (25.0/5.0 mL) was added Fe (3.8 g, 68.66 mmol) and NH₄Cl (3.6 g, 68.66 mmol) at room temperature under N₂. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with DCM/MeOH (95/5, v/v) to afford 4-bromo-3-methoxypyridin-2-amine (2.6 g, 93%) as a purple solid. LCMS (ESI, m/z): [M+H]⁺=203.0.

### Step 4: Synthesis of (E)-N′-(4-bromo-3-methoxypyridin-2-yl)-N,N-dimethylmethanimidamide

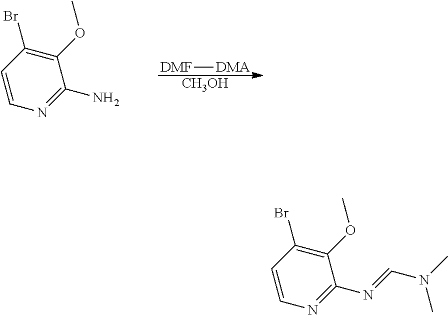

To a solution of 4-bromo-3-methoxypyridin-2-amine (2.5 g, 12.31 mmol) in CH₃OH (40.0 mL) was added DMF-DMA (1.8 g, 14.94 mmol) at room temperature under N₂. The resulting mixture was stirred at 70° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (45/55, v/v) to afford (E)-N′-(4-bromo-3-methoxypyridin-2-yl)-N,N-dimethylmethanimidamide (2.4 g, 75%) as a white solid. LCMS (ESI, m/z): [M+H]⁺=258.0.

### Step 5: Synthesis of (E)-N′-(4-bromo-3-methoxypyridin-2-yl)-N-hydroxymethanimidamide

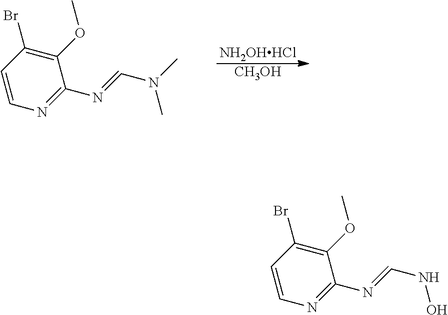

To a solution of (E)-N′-(4-bromo-3-methoxypyridin-2-yl)-N,N-dimethylmethanimidamide (2.4 g, 9.30 mmol) in CH₃OH (20.0 mL) was added NH₂OH HCl (2.4 g, 34.51 mmol) at room temperature under N₂. The resulting mixture was stirred at 70° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (77/23, v/v) to afford (E)-N′-(4-bromo-3-methoxypyridin-2-yl)-N-hydroxymethanimidamide (2.1 g, 92%) as a white solid. LCMS (ESI, m/z): [M+H]⁺=246.0.

### Step 6: Synthesis of 7-bromo-8-methoxy-[1,2,4]triazolo[1,5-a]pyridine

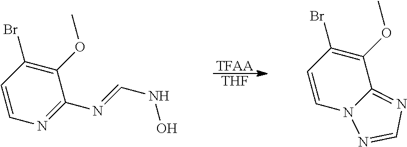

To a solution of (E)-N′-(4-bromo-3-methoxypyridin-2-yl)-N-hydroxymethanimidamide (1.0 g, 4.06 mmol) in THF (5.0 mL) was added TFAA (938.9 mg, 4.47 mmol) at room temperature under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 7-bromo-8-methoxy-[1,2,4]triazolo[1,5-a]pyridine (1.2 g, crude) as a brown solid. LCMS (ESI, m/z): [M+H]⁺=228.0.

### Step 7: Synthesis of 6-chloro-3-[8-methoxy-[1,2,4]triazolo[1,5-a]pyridin-7-yl]-1-[[2-(trimethylsilyl)ethoxy]methyl]pyrrolo[2,3-b]pyridine

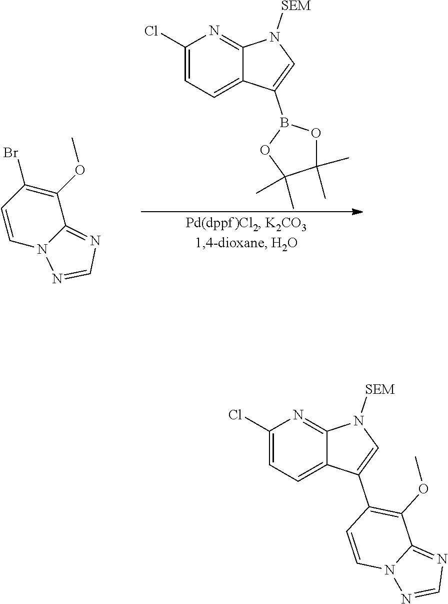 [SMILES: COc1c(Br)ccn2ncnc12 ; CC1(C)OB(c2cn(COCC[Si](C)(C)C)c3nc(Cl)ccc23)OC1(C)C ; COc1c(-c2cn(COCC[Si](C)(C)C)c3nc(Cl)ccc23)ccn2ncnc12]

To a solution of 7-bromo-8-methoxy-[1,2,4]triazolo[1,5-a]pyridine (1.2 g, 5.26 mmol) in 1,4-dioxane/H₂O (5.0/1.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (2.4 g, 5.87 mmol), K₂CO₃ (2.1 g, 15.19 mmol) and Pd(dppf)Cl₂ (420.0 mg, 0.54 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 6-chloro-3-[8-methoxy-[1,2,4]triazolo[1,5-a]pyridin-7-yl]-1-[[2-(trimethylsilyl)ethoxy]methyl]pyrrolo[2,3-b]pyridine (1.2 g, 52%) as a white solid. LCMS (ESI, m/z): [M+H]⁺=430.1.

### Step 8: Synthesis of (1S,2S)-2-fluoro-N-(3-[8-methoxy-[1,2,4]triazolo[1,5-a]pyridin-7-yl]-1-[[2-(trimethylsilyl)ethoxy]methyl]pyrrolo[2,3-b]pyridin-6-yl)cyclopropane-1-carboxamide

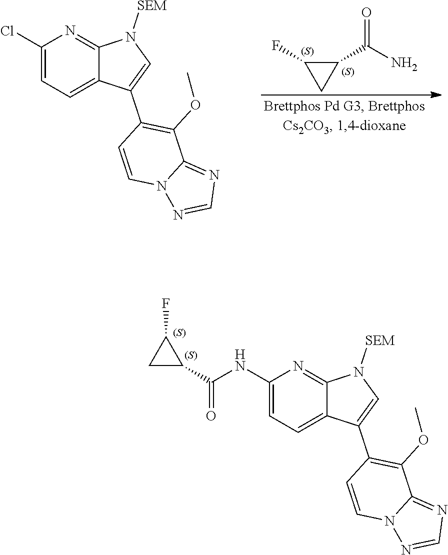

To a solution of 6-chloro-3-[8-methoxy-[1,2,4]triazolo[1,5-a]pyridin-7-yl]-1-[[2-(trimethylsilyl)ethoxy]methyl]pyrrolo[2,3-b]pyridine (300.0 mg, 0.68 mmol) in 1,4-dioxane (5.0 mL) was added (1S,2S)-2-fluorocyclopropane-1-carboxamide (360.0 mg, 3.49 mmol), Cs₂CO₃ (681.9 mg, 2.01 mmol), Brettphos (75.0 mg, 0.14 mmol) and Brettphos Pd G3 (63.2 mg, 0.07 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (68/32, v/v) to afford (1S,2S)-2-fluoro-N-(3-[8-methoxy-[1,2,4]triazolo[1,5-a]pyridin-7-yl]-1-[[2-(trimethylsilyl)ethoxy]methyl]pyrrolo[2,3-b]pyridin-6-yl)cyclopropane-1-carboxamide (122.0 mg, 36%) as an off-white solid. LCMS (ESI, m/z): [M+H]⁺=497.2.

### Step 9: Synthesis of (1S,2S)-2-fluoro-N-(3-[8-methoxy-[1,2,4]triazolo[1,5-a]pyridin-7-yl]-1H-pyrrolo[2,3-b]pyridin-6-yl)cyclopropane-1-carboxamide (Compound 115)

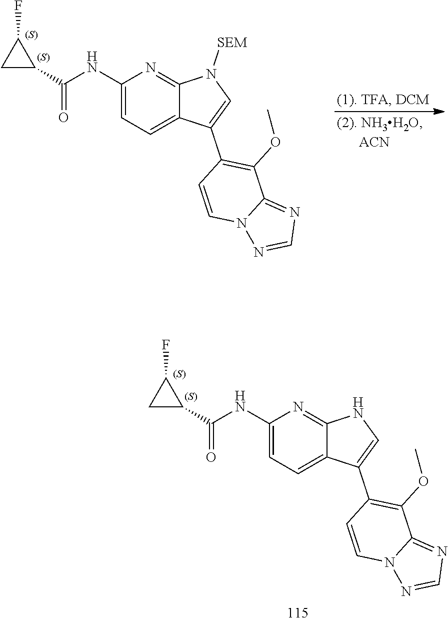

To a solution of (1S,2S)-2-fluoro-N-(3-[8-methoxy-[1,2,4]triazolo[1,5-a]pyridin-7-yl]-1-[[2-(trimethylsilyl)ethoxy]methyl]pyrrolo[2,3-b]pyridin-6-yl)cyclopropane-1-carboxamide (60.0 mg, 0.12 mmol) in DCM (3.0 mL) was added TFA (3.0 mL) at room temperature. The resulting mixture was stirred at room temperature for 3 h. The resulting mixture was concentrated under reduced pressure. The residue was dissolved in ACN (3.0 mL) and NH₃·H₂O (3.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 11% B to 41% B in 9 min; 254 nm) to afford (1S,2S)-2-fluoro-N-(3-[8-methoxy-[1,2,4]triazolo[1,5-a]pyridin-7-yl]-1H-pyrrolo[2,3-b]pyridin-6-yl)cyclopropane-1-carboxamide (14.4 mg, 32%) as a white solid. LCMS (ESI, m/z): [M+H]⁺=367.0. ¹H NMR (400 MHz, DMSO-d₆): δ 11.93 (s, 1H), 10.71 (s, 1H), 8.72 (d, J=7.2 Hz, 1H), 8.48 (s, 1H), 8.16 (d, J=8.8 Hz, 1H), 7.97 (d, J=8.4 Hz, 1H), 7.86 (s, 1H), 7.45 (d, J=7.2 Hz, 1H), 5.04-4.83 (m, 1H), 4.20 (s, 3H), 2.26-2.23 (m, 1H), 1.71-1.64 (m, 1H), 1.26-1.10 (m, 1H).

### Example S116. Compound 116

### Step 1: Synthesis of 3-(6-chloro-1-[[2-(trimethylsilyl)ethoxy]methyl]pyrrolo[2,3-b]pyridin-3-yl)-5-fluoro-2,4-dimethoxypyridine

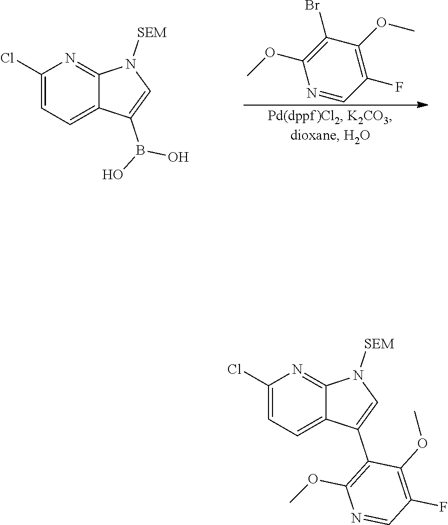

To a solution of 6-chloro-1-[[2-(trimethylsilyl)ethoxy]methyl]pyrrolo[2,3-b]pyridin-3-ylboronic acid (300.0 mg, 0.92 mmol) in 1,4-dioxane/H₂O (15.0 mL/3.0 mL) was added 3-bromo-5-fluoro-2,4-dimethoxypyridine (216.8 mg, 0.92 mmol), K₂CO₃ (253.9 mg, 1.84 mmol) and Pd(dppf)Cl₂ (67.2 mg, 0.09 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 3-(6-chloro-1-[[2-(trimethylsilyl)ethoxy]methyl]pyrrolo[2,3-b]pyridin-3-yl)-5-fluoro-2,4-dimethoxypyridine (277.9 mg, 69%) as a light yellow oil. LCMS (ESI, m/z): [M+H]⁺=438.1.

### Step 2: Synthesis of N-[3-(5-fluoro-2,4-dimethoxypyridin-3-yl)-1-[[2-(trimethylsilyl)ethoxy]methyl]pyrrolo[2,3-b]pyridin-6-yl]-1,1-diphenylmethanimine

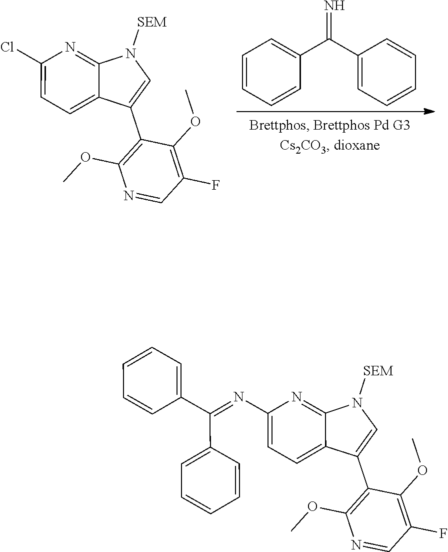 [SMILES: COc1ncc(F)c(OC)c1-c1cn(COCC[Si](C)(C)C)c2nc(Cl)ccc12 ; N=C(c1ccccc1)c1ccccc1 ; COc1ncc(F)c(OC)c1-c1cn(COCC[Si](C)(C)C)c2nc(N=C(c3ccccc3)c3ccccc3)ccc12]

To a solution of 3-(6-chloro-1-[[2-(trimethylsilyl)ethoxy]methyl]pyrrolo[2,3-b]pyridin-3-yl)-5-fluoro-2,4-dimethoxypyridine (213.0 mg, 0.49 mmol) in 1,4-dioxane (16.0 mL) was added diphenylmethanimine (440.7 mg, 2.43 mmol), BrettPhos (52.2 mg, 0.10 mmol), Cs₂CO₃ (475.4 mg, 1.46 mmol) and BrettPhos Pd G3 (44.1 mg, 0.05 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford N-[3-(5-fluoro-2,4-dimethoxypyridin-3-yl)-1-[[2-(trimethylsilyl)ethoxy]methyl]pyrrolo[2,3-b]pyridin-6-yl]-1,1-diphenylmethanimine (146.9 mg, 51%) as a light yellow oil. LCMS (ESI, m/z): [M+H]⁺=583.2.

### Step 3: Synthesis of 3-(5-fluoro-2,4-dimethoxypyridin-3-yl)-1-[[2-(trimethylsilyl)ethoxy]methyl]pyrrolo[2,3-b]pyridin-6-amine

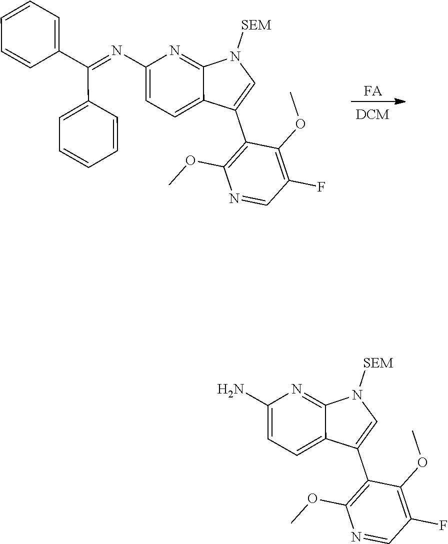 [SMILES: COc1ncc(F)c(OC)c1-c1cn(COCC[Si](C)(C)C)c2nc(N=C(c3ccccc3)c3ccccc3)ccc12 ; "FA" ; COc1ncc(F)c(OC)c1-c1cn(COCC[Si](C)(C)C)c2nc(N)ccc12]

To a solution of N-[3-(5-fluoro-2,4-dimethoxypyridin-3-yl)-1-[[2-(trimethylsilyl)ethoxy]methyl]pyrrolo[2,3-b]pyridin-6-yl]-1,1-diphenylmethanimine (123.5 mg, 0.21 mmol) in CH₂Cl₂ (6.0 mL) was added FA (6.0 mL) at room temperature. The resulting mixture was stirred at room temperature for 3 h. After the reaction was completed, The pH value of the mixture was adjusted to 8 with aq·NaHCO₃. The resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 3-(5-fluoro-2,4-dimethoxypyridin-3-yl)-1-[[2-(trimethylsilyl)ethoxy]methyl]pyrrolo[2,3-b]pyridin-6-amine (46.3 mg, 52%) as a light yellow oil. LCMS (ESI, m/z): [M+H]⁺=419.2.

### Step 4: Synthesis of 3-[3-(5-fluoro-2,4-dimethoxypyridin-3-yl)-1-[[2-(trimethylsilyl)ethoxy]methyl]pyrrolo[2,3-b]pyridin-6-yl]-1-[2-(4-methylpiperazin-1-yl)ethyl]urea

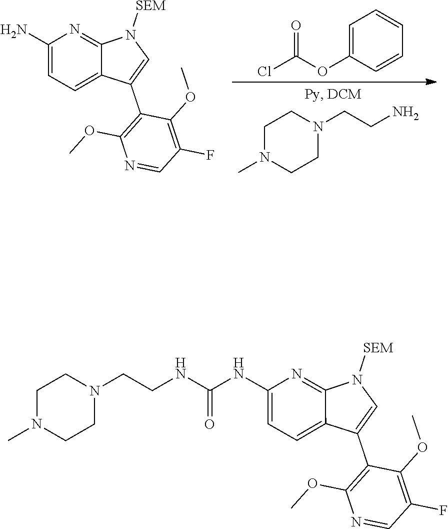

To a solution of 3-(5-fluoro-2,4-dimethoxypyridin-3-yl)-1-[[2-(trimethylsilyl)ethoxy]methyl]pyrrolo[2,3-b]pyridin-6-amine (209.0 mg, 0.50 mmol) in DCM (9.0 mL) was added pyridine (158.0 mg, 2.00 mmol) and phenyl chloroformate (93.8 mg, 0.60 mmol) at room temperature. The resulting mixture was stirred at room temperature for 16 h. The resulting mixture was concentrated under reduced pressure. To the above mixture was added 2-(4-methylpiperazin-1-yl)ethanamine (214.6 mg, 1.50 mmol) and pyridine (9.0 mL) at room temperature. The resulting mixture was stirred at 60° C. for 2 h. After the reaction was completed, the resulting mixture was concentrated under reduced pressure. The residue was purified by flash column chromatography with methylene chloride/methanol (90/10, v/v) to afford 3-[3-(5-fluoro-2,4-dimethoxypyridin-3-yl)-1-[[2-(trimethylsilyl)ethoxy]methyl]pyrrolo[2,3-b]pyridin-6-yl]-1-[2-(4-methylpiperazin-1-yl)ethyl]urea (99.1 mg, 33%) as a light brown oil. LCMS (ESI, m/z): [M+H]⁺=588.3.

### Step 5: Synthesis of 3-[3-(5-fluoro-2,4-dimethoxypyridin-3-yl)-1H-pyrrolo[2,3-b]pyridin-6-yl]-1-[2-(4-methylpiperazin-1-yl)ethyl]urea (Compound 116)

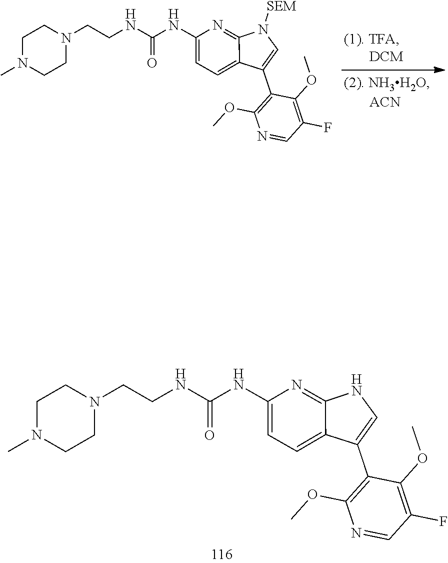 [SMILES: COc1ncc(F)c(OC)c1-c1cn(COCC[Si](C)(C)C)c2nc(NC(=O)NCCN3CCN(C)CC3)ccc12 ; COc1ncc(F)c(OC)c1-c1c[nH]c2nc(NC(=O)NCCN3CCN(C)CC3)ccc12]

To a solution of 3-[3-(5-fluoro-2,4-dimethoxypyridin-3-yl)-1-[[2-(trimethylsilyl)ethoxy]methyl]pyrrolo[2,3-b]pyridin-6-yl]-1-[2-(4-methylpiperazin-1-yl)ethyl]urea (99.1 mg, 0.169 mmol) in CH₂Cl₂ (3.0 mL) was added TFA (3.0 mL) at room temperature. The resulting mixture was stirred at room temperature for 3 h. The resulting mixture was concentrated under reduced pressure. The residue was dissolved in CH₃CN (3.0 mL) and NH₃·H₂O (3.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 7 min; 254 nm) to afford 3-[3-(5-fluoro-2,4-dimethoxypyridin-3-yl)-1H-pyrrolo[2,3-b]pyridin-6-yl]-1-[2-(4-methylpiperazin-1-yl)ethyl]urea (10.6 mg, 13%) as a white solid. LCMS (ESI, m/z): [M+H]⁺=458.3. ¹H NMR (400 MHz, DMSO-d₆): δ 11.55 (s, 11H), 9.19 (s, 11H), 8.27 (s, 11H), 8.10 (d, J=2.8 Hz, 11H), 7.62 (d, J=8.8 Hz, 11H), 7.35 (s, 1H), 7.08 (d, J=8.8 Hz, 1H), 3.79-3.78 (m, 6H), 2.54-2.44 (m, 6H), 2.32 (s, 4H), 2.15 (s, 3H).

### Example S117. Compound 117

### Step 1: Synthesis of 3-(2,6-dimethoxyphenyl)-1-[[2-(trimethylsilyl)ethoxy]methyl]pyrrolo[2,3-b]pyridin-6-amine

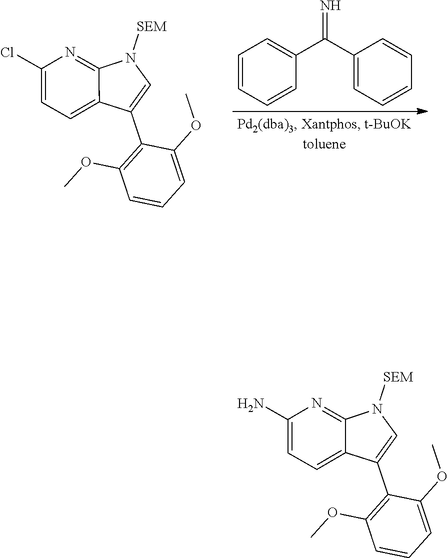 [SMILES: COc1cccc(OC)c1-c1cn(COCC[Si](C)(C)C)c2nc(Cl)ccc12 ; N=C(c1ccccc1)c1ccccc1 ; COc1cccc(OC)c1-c1cn(COCC[Si](C)(C)C)c2nc(N)ccc12]

To a solution of 6-chloro-3-(2,6-dimethoxyphenyl)-1-((2-(trimethylsilyl)ethoxy)methyl)-1H-pyrrolo[2,3-b]pyridine (270.0 mg, 0.64 mmol) in toluene (12.0 mL) was added diphenylmethanimine (350.4 mg, 1.93 mmol), XantPhos (74.6 mg, 0.13 mmol), t-BuOK (216.9 mg, 1.93 mmol) and Pd₂(dba)₃ (118.0 mg, 0.13 mmol) at room temperature under N₂. The resulting mixture was stirred at 100° C. for 16 h. The resulting mixture was concentrated under reduced pressure. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 3-(2,6-dimethoxyphenyl)-1-[[2-(trimethylsilyl)ethoxy]methyl]pyrrolo[2,3-b]pyridin-6-amine (135.5 mg, 53%) as a light brown oil. LCMS (ESI, m/z): [M+H]⁺=400.2.

### Step 2: Synthesis of 3-[3-(2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea

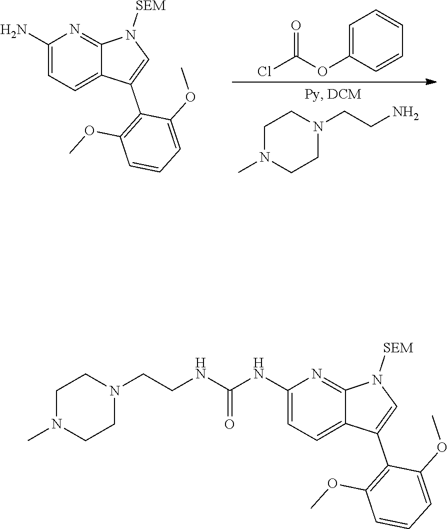

To a solution of 3-(2,6-dimethoxyphenyl)-1-[[2-(trimethylsilyl)ethoxy]methyl]pyrrolo[2,3-b]pyridin-6-amine (180.0 mg, 0.45 mmol) in DCM (8.0 mL) was added Pyridine (142.5 mg, 1.80 mmol) and phenyl chloroformate (84.6 mg, 0.54 mmol) at room temperature. The resulting mixture was stirred at room temperature for 16 h. The resulting mixture was concentrated under reduced pressure. To the above residue was added 2-(4-methylpiperazin-1-yl)ethanamine (193.6 mg, 1.35 mmol) and pyridine (8.0 mL) at room temperature. The resulting mixture was stirred at 60° C. for another 2 h. After the reaction was completed, the resulting mixture was concentrated under reduced pressure. The residue was purified by flash column chromatography with methylene chloride/methanol (9/1, v/v) to afford 3-[3-(2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea (209.2 mg, 82%) as a light brown oil. LCMS (ESI, m/z): [M+H]⁺=569.3.

### Step 3: Synthesis of 3-[3-(2,6-dimethoxyphenyl)-1H-pyrrolo[2,3-b]pyridin-6-yl]-1-[2-(4-methylpiperazin-1-yl)ethyl]urea (Compound 117)

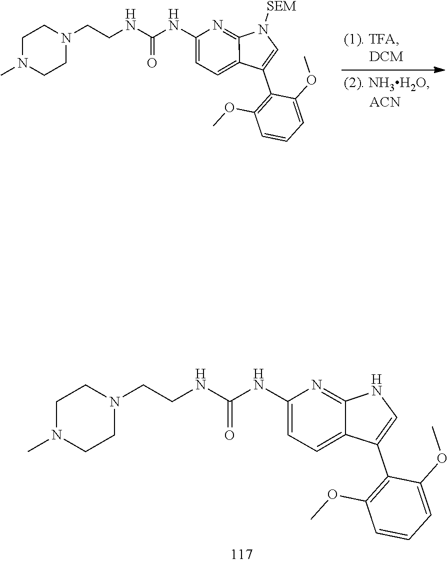 [SMILES: COc1cccc(OC)c1-c1cn(COCC[Si](C)(C)C)c2nc(NC(=O)NCCN3CCN(C)CC3)ccc12 ; COc1cccc(OC)c1-c1c[nH]c2nc(NC(=O)NCCN3CCN(C)CC3)ccc12]

To a solution of 3-[3-(2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea (209.2 mg, 0.37 mmol) in CH₂Cl₂ (6.0 mL) was added TFA (6.0 mL) at room temperature. The resulting mixture was stirred at room temperature for 16 h. The resulting mixture was concentrated under reduced pressure. To the residue in CH₃CN (6.0 mL) was added NH₃·H₂O (6.0 mL) at room temperature. The resulting mixture was stirred at room temperature for another 3 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10.0 mmol/L NH₄HCO₃), Mobile Phase B: ACN; Flow rate: 60 mL/min; Gradient: 17% B to 47% B in 7 min; 254 nm) to afford 3-[3-(2,6-dimethoxyphenyl)-1H-pyrrolo[2,3-b]pyridin-6-yl]-1-[2-(4-methylpiperazin-1-yl)ethyl]urea (14.3 mg, 8%) as a white solid. LCMS (ESI, m/z): [M+H]⁺=439.2. ¹H NMR (400 MHz, DMSO-d₆): δ 11.28 (s, 1H), 9.13 (s, 1H), 8.37 (s, 1H), 7.46 (d, J=8.4 Hz, 1H), 7.28-7.24 (m, 1H), 7.16 (d, J=2.0 Hz, 1H), 6.99 (d, J=8.4 Hz, 1H), 6.73 (d, J=8.4 Hz, 2H), 3.68 (s, 6H), 2.50-2.44 (m, 6H), 2.32 (s, 4H), 2.15 (s, 3H).

### Example S118. Compound 118

### Step 1: Synthesis of (1S,2S)—N-[3-(2,6-dimethoxyphenyl)-1-[[2-(trimethylsilyl)ethoxy]methyl]pyrrolo[2,3-b]pyridin-6-yl]-2-fluorocyclopropane-1-carboxamide

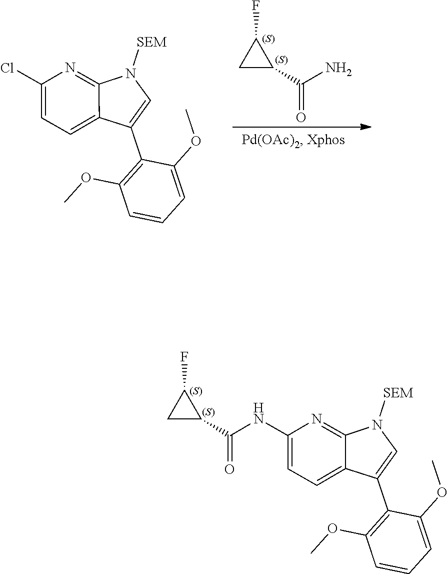

To a solution of 6-chloro-3-(2,6-dimethoxyphenyl)-1-[[2-(trimethylsilyl)ethoxy]methyl]pyrrolo[2,3-b]pyridine (280.0 mg, 0.67 mmol) in dioxane (7.5 mL) was added (1S,2S)-2-fluorocyclopropane-1-carboxamide (344.5 mg, 3.34 mmol), K₂CO₃ (277.1 mg, 2.0 mmol), XPhos (63.7 mg, 0.13 mmol) and Pd(OAc)₂ (15.0 mg, 0.07 mmol) under N₂. The mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford (1S,2S)—N-[3-(2,6-dimethoxyphenyl)-1-[[2-(trimethylsilyl)ethoxy]methyl]pyrrolo[2,3-b]pyridin-6-yl]-2-fluorocyclopropane-1-carboxamide (129.0 mg, 39%) as a yellow solid. LCMS (ESI, m/z): [M+H]⁺=486.2.

### Step 2: Synthesis of (1S,2S)—N-[3-(2,6-dimethoxyphenyl)-1H-pyrrolo[2,3-b]pyridin-6-yl]-2-fluorocyclopropane-1-carboxamide (Compound 118)

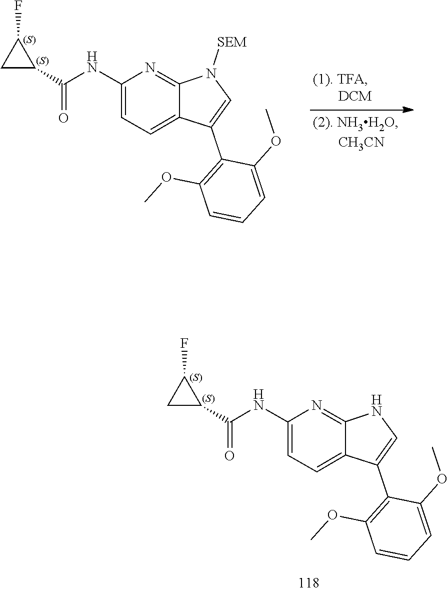

To a solution of (1S,2S)—N-[3-(2,6-dimethoxyphenyl)-1-[[2-(trimethylsilyl)ethoxy]methyl]pyrrolo[2,3-b]pyridin-6-yl]-2-fluorocyclopropane-1-carboxamide (109.0 mg, 0.22 mmol) in CH₂Cl₂ (6.0 mL) was added TFA (3.0 mL) at room temperature. The reaction mixture was stirred at room temperature for 4 h. The mixture was concentrated under vacuum. The residue was dissolved in CH₃CN (5.0 mL) and NH₃·H₂O (5.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 34% B to 64% B in 7 min; 254 nm, RT1: 6.5 min to afford (1S,2S)—N-[3-(2,6-dimethoxyphenyl)-1H-pyrrolo[2,3-b]pyridin-6-yl]-2-fluorocyclopropane-1-carboxamide (24.9 mg, 30%) as a white solid. LCMS (ESI, m/z): [M+H]⁺=356.0. ¹H NMR (300 MHz, DMSO-d₆): δ 11.43 (s, 1H), 10.58 (s, 1H), 7.82 (d, J=8.4 Hz, 1H), 7.55 (d, J=8.7 Hz, 1H), 7.31-7.25 (m, 2H), 6.75 (d, J=8.4 Hz, 2H), 5.04-4.80 (m, 1H), 3.70 (s, 6H), 2.25-2.20 (m, 1H), 1.70-1.60 (m, 1H), 1.18-1.11 (m, 1H).

### Example S119. Compound 119

### Step 1: Synthesis of 6-chloro-3-(3-fluoro-2,6-dimethoxyphenyl)-1-[[2-(trimethylsilyl)ethoxy]methyl]pyrrolo[2,3-b]pyridine

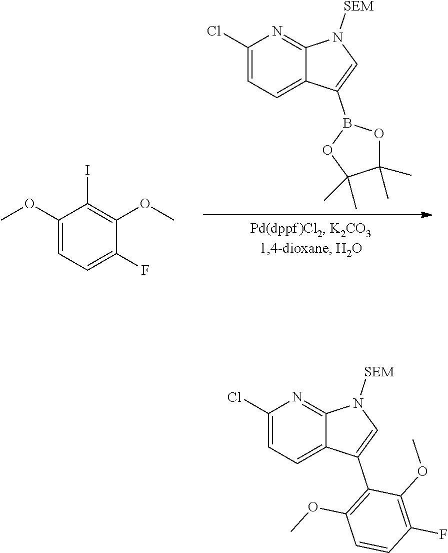

To a solution of 1-fluoro-3-iodo-2,4-dimethoxybenzene (1.0 g, 3.55 mmol) in 1,4-dioxane (18.0 mL) and H₂O (3.6 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (1.5 g, 3.55 mmol), K₂CO₃ (1.0 g, 0.01 mmol) and Pd(dppf)Cl₂ (0.3 g, 0.36 mmol) under N₂. The mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (10/1, v/v) to afford 6-chloro-3-(3-fluoro-2,6-dimethoxyphenyl)-1-[[2-(trimethylsilyl)ethoxy]methyl]pyrrolo[2,3-b]pyridine (1.0 g, 64%) as a yellow solid. LCMS (ESI, m/z): [M+H]⁺=437.1.

### Step 2: Synthesis of (1S,2S)-2-fluoro-N-[3-(3-fluoro-2,6-dimethoxyphenyl)-1-[[2-(trimethylsilyl)ethoxy]methyl]pyrrolo[2,3-b]pyridin-6-yl]cyclopropane-1-carboxamide

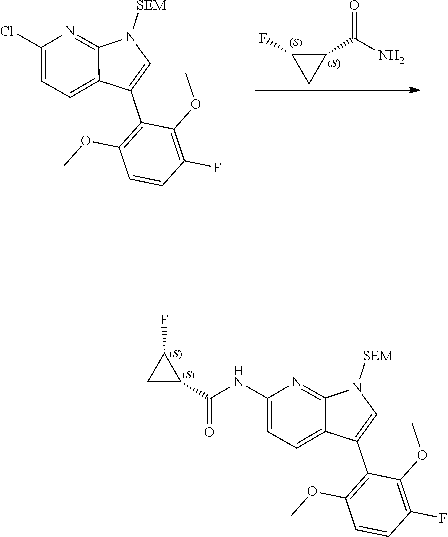

To a solution of 6-chloro-3-(3-fluoro-2,6-dimethoxyphenyl)-1-[[2-(trimethylsilyl)ethoxy]methyl]pyrrolo[2,3-b]pyridine (300.0 mg, 0.69 mmol) in t-BuOH (10.0 mL) was added (1S,2S)-2-fluorocyclopropane-1-carboxamide (353.9 mg, 3.43 mmol), K₂CO₃ (284.7 mg, 2.06 mmol), XPhos (65.5 mg, 0.14 mmol) and Pd(OAc)₂ (15.4 mg, 0.07 mmol) under N₂. The mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford (1S,2S)-2-fluoro-N-[3-(3-fluoro-2,6-dimethoxyphenyl)-1-[[2-(trimethylsilyl)ethoxy]methyl]pyrrolo[2,3-b]pyridin-6-yl]cyclopropane-1-carboxamide (279.0 mg, 55%) as a yellow solid. LCMS (ESI, m/z): [M+H]⁺=504.2.

### Step 3: Synthesis of (1S,2S)-2-fluoro-N-[3-(3-fluoro-2,6-dimethoxyphenyl)-1H-pyrrolo[2,3-b]pyridin-6-yl]cyclopropane-1-carboxamide (Compound 119)

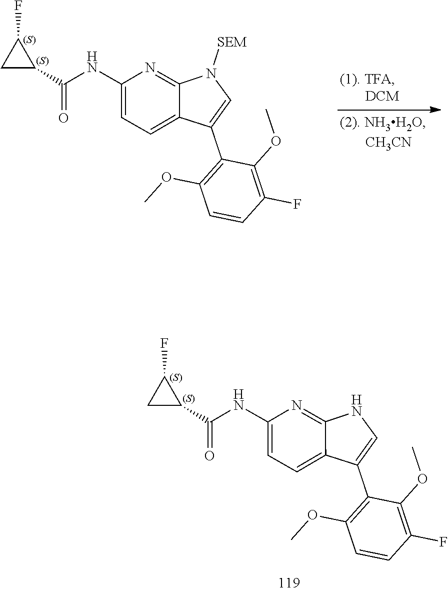

To a solution of (1S,2S)-2-fluoro-N-[3-(3-fluoro-2,6-dimethoxyphenyl)-1-[[2-(trimethylsilyl)ethoxy]methyl]pyrrolo[2,3-b]pyridin-6-yl]cyclopropane-1-carboxamide (279.0 mg, 0.56 mmol) in CH₂Cl₂ (14.0 mL) was added TFA (14.0 mL) at room temperature. The reaction mixture was stirred at room temperature for 4 h. The mixture was concentrated under vacuum. The residue was dissolved in CH₃CN (15.0 mL) and NH₃·H₂O (15.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 41% B to 51% B in 9 min; 254/220 nm; to afford (1S,2S)-2-fluoro-N-[3-(3-fluoro-2,6-dimethoxyphenyl)-1H-pyrrolo[2,3-b]pyridin-6-yl]cyclopropane-1-carboxamide (78.0 mg, 37%) as a white solid. LCMS (ESI, m/z): [M+H]⁺=374.0. ¹H NMR (300 MHz, DMSO-d₆): δ 11.60 (d, J=1.8 Hz, 1H), 10.62 (s, 1H), 7.84 (d, J=8.4 Hz, 1H), 7.65 (d, J=8.4 Hz, 1H), 7.40 (d, J=2.4 Hz, 1H), 7.24-7.17 (m, 1H), 6.85-6.80 (m, 1H), 5.06-4.78 (m, 1H), 3.68 (s, 3H), 3.53 (s, 3H), 2.25-2.20 (m, 1H), 1.70-1.59 (m, 1H), 1.18-1.11 (m, 1H)

### Example S120. Compound 120

### Step 1: Synthesis of 1,5-difluoro-3-iodo-2,4-dimethoxybenzene

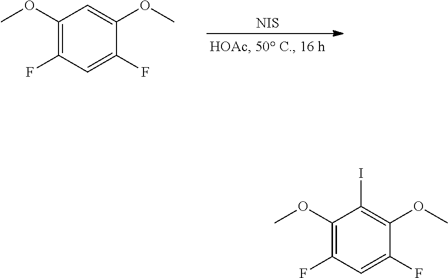

To a solution of 1,5-difluoro-2,4-dimethoxybenzene (500.0 mg, 2.87 mmol) in HOAc (10.0 mL) was added NIS (1.3 g, 5.74 mmol) at room temperature. The mixture was stirred at 50° C. for 16 h. After the reaction was completed, the mixture was concentrated under vacuum. The residue was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (5/1, v/v) to afford 1,5-difluoro-3-iodo-2,4-dimethoxybenzene (386.0 mg, 44%) as a white solid.

### Step 2: Synthesis of 6-chloro-3-(3,5-difluoro-2,6-dimethoxyphenyl)-1-[[2-(trimethylsilyl)ethoxy]methyl]pyrrolo[2,3-b]pyridine

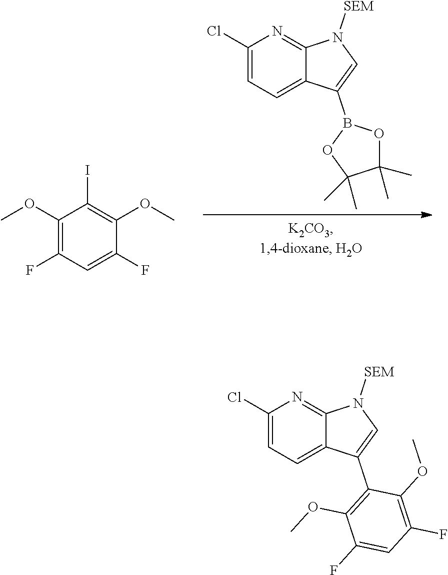

To a solution of 1,5-difluoro-3-iodo-2,4-dimethoxybenzene (500.0 mg, 1.67 mmol) in 1,4-dioxane (10.0 mL) and H₂O (2.0 mL) were added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (681.2 mg, 1.67 mmol), Pd(PPh₃)₄ (385.1 mg, 0.33 mmol) and K₂CO₃ (460.6 mg, 3.33 mmol) at room temperature under N₂. The mixture was stirred at 100° C. for 12 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (10/1, v/v) to afford 6-chloro-3-(3,5-difluoro-2,6-dimethoxyphenyl)-1-[[2-(trimethylsilyl)ethoxy]methyl]pyrrolo[2,3-b]pyridine (321.0 mg, 42%) as a white solid. LCMS (ESI, m/z): [M+H]⁺=455.1.

### Step 3: Synthesis of (1S,2S)—N-[3-(3,5-difluoro-2,6-dimethoxyphenyl)-1-[[2-(trimethylsilyl)ethoxy]methyl]pyrrolo[2,3-b]pyridin-6-yl]-2-fluorocyclopropane-1-carboxamide

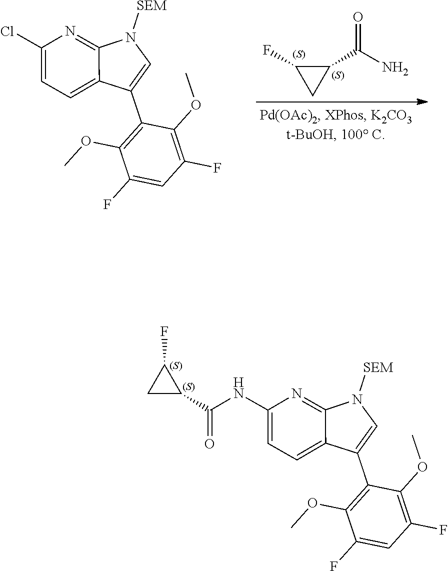

To a solution of 6-chloro-3-(3,5-difluoro-2,6-dimethoxyphenyl)-1-[[2-(trimethylsilyl)ethoxy]methyl]pyrrolo[2,3-b]pyridine (260.0 mg, 0.57 mmol) in t-BuOH (5.5 mL) were added (1S,2S)-2-fluorocyclopropane-1-carboxamide (294.6 mg, 2.86 mmol), K₂CO₃ (236.9 mg, 1.71 mmol), Pd(OAc)₂ (12.8 mg, 0.06 mmol) and XPhos (54.5 mg, 0.11 mmol) at room temperature under N₂. The mixture was stirred at 100° C. for 12 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford (1S,2S)—N-[3-(3,5-difluoro-2,6-dimethoxyphenyl)-1-[[2-(trimethylsilyl)ethoxy]methyl]pyrrolo[2,3-b]pyridin-6-yl]-2-fluorocyclopropane-1-carboxamide (70.0 mg, 23%) as a yellow solid. LCMS (ESI, m/z): [M+H]⁺=522.2.

### Step 4: Synthesis of (1S,2S)—N-[3-(3,5-difluoro-2,6-dimethoxyphenyl)-1H-pyrrolo[2,3-b]pyridin-6-yl]-2-fluorocyclopropane-1-carboxamide (Compound 120)

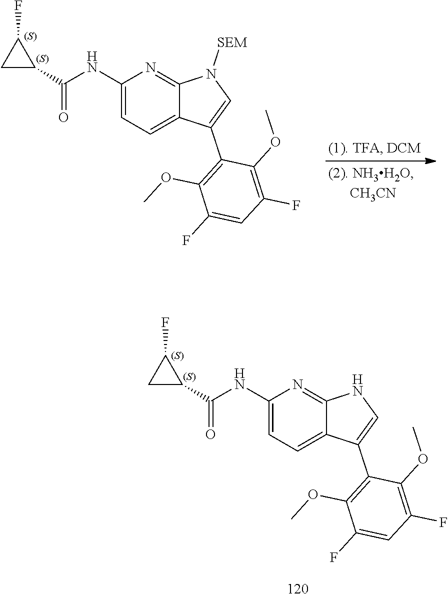

To a solution of (1S,2S)—N-[3-(3,5-difluoro-2,6-dimethoxyphenyl)-1-[[2-(trimethylsilyl)ethoxy]methyl]pyrrolo[2,3-b]pyridin-6-yl]-2-fluorocyclopropane-1-carboxamide (60.0 mg, 0.12 mmol) in CH₂Cl₂ (2.0 mL) was added TFA (2.0 mL) at room temperature. The reaction mixture was stirred at room temperature for 4 h. The mixture was concentrated under vacuum. The residue was dissolved in CH₃CN (4.0 mL) and NH₃·H₂O (4.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 42% B to 52% B in 9 min to afford (1S,2S)—N-[3-(3,5-difluoro-2,6-dimethoxyphenyl)-1H-pyrrolo[2,3-b]pyridin-6-yl]-2-fluorocyclopropane-1-carboxamide (14.9 mg, 32%) as a white solid. LCMS (ESI, m/z): [M+H]⁺=392.0. ¹H NMR (300 MHz, DMSO-d₆): δ 11.77 (s, 1H), 10.66 (s, 1H), 7.88 (d, J=8.7 Hz, 1H), 7.76 (d, J=8.4 Hz, 1H), 7.52 (d, J=2.4 Hz, 1H), 7.46-7.38 (m, 1H), 5.07-4.79 (m, 1H), 3.50 (s, 6H), 2.29-2.20 (m, 1H), 1.72-1.59 (m, 1H), 1.21-1.10 (m, 1H).

### Example S121. Compound 121

### Step 1: Synthesis of tert-butyl 4-[2-([[3-(2-methoxyphenyl)-1-[[2-(trimethylsilyl)ethoxy]methyl]pyrrolo[2,3-b]pyridin-6-yl]carbamoyl]amino)ethyl]piperazine-1-carboxylate

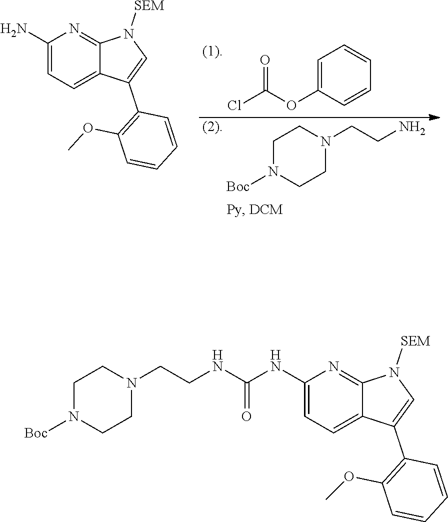

To a solution of 3-(2-methoxyphenyl)-1-[[2-(trimethylsilyl)ethoxy]methyl]pyrrolo[2,3-b]pyridin-6-amine (150.0 mg, 0.41 mmol) in CH₂Cl₂ (3.0 mL) was added pyridine (128.4 mg, 1.62 mmol) and phenyl chloroformate (76.3 mg, 0.49 mmol) at 0° C. The mixture was stirred at room temperature for 16 h. The mixture was concentrated under vacuum. To the above residue was added pyridine (3.0 mL) and tert-butyl 4-(2-aminoethyl)piperazine-1-carboxylate (279.3 mg, 1.22 mmol). The resulting mixture was stirred at 60° C. for another 2 h. After the reaction was completed, the resulting mixture was concentrated under vacuum. The residue was purified by flash column chromatography with petroleum ether/ethyl acetate (5/95, v/v) to afford tert-butyl 4-[2-([[3-(2-methoxyphenyl)-1-[[2-(trimethylsilyl)ethoxy]methyl]pyrrolo[2,3-b]pyridin-6-yl]carbamoyl]amino)ethyl]piperazine-1-carboxylate (190.0 mg, 74%) as a yellow solid. LCMS (ESI, m/z): [M+H]⁺=625.3.

### Step 2: Synthesis of 3-[3-(2-methoxyphenyl)-1H-pyrrolo[2,3-b]pyridin-6-yl]-1-[2-(piperazin-1-yl)ethyl]urea (Compound 121)

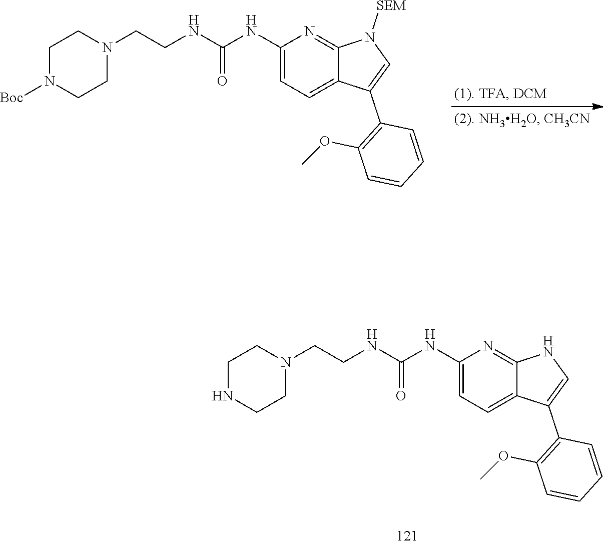 [SMILES: COc1ccccc1-c1cn(COCC[Si](C)(C)C)c2nc(NC(=O)NCCN3CCN(C(=O)OC(C)(C)C)CC3)ccc12 ; COc1ccccc1-c1c[nH]c2nc(NC(=O)NCCN3CCNCC3)ccc12]

To a solution of tert-butyl 4-[2-([[3-(2-methoxyphenyl)-1-[[2-(trimethylsilyl)ethoxy]methyl]pyrrolo[2,3-b]pyridin-6-yl]carbamoyl]amino)ethyl]piperazine-1-carboxylate (160.0 mg, 0.26 mmol) in CH₂Cl₂ (3.0 mL) was added TFA (3.0 mL). The mixture was stirred at room temperature for 2 h. The mixture was concentrated under vacuum. To the above residue was added ACN (3.0 mL) and NH₃·H₂O (3.0 mL). The resulting mixture was stirred at room temperature for another 2 h. After the reaction was completed, the resulting mixture was concentrated under vacuum. The residue was purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 25% B to 55% B in 7 min; 254 nm) to afford 3-[3-(2-methoxyphenyl)-1H-pyrrolo[2,3-b]pyridin-6-yl]-1-[2-(piperazin-1-yl)ethyl]urea (48.0 mg, 47%) as a white solid. LCMS (ESI, m/z): [M+H]⁺=395.2. ¹H NMR (400 MHz, DMSO-d₆): δ 11.43 (s, 1H), 9.19 (s, 1H), 8.32 (s, 1H), 7.90 (d, J=8.8 Hz, 1H), 7.52-7.45 (m, 2H), 7.28-7.23 (m, 1H), 7.11-6.99 (m, 3H), 3.81 (s, 3H), 2.51-2.36 (m, 12H).

### Example S122. Compound 122

### Step 1: Synthesis of 3-(cyclopropylmethyl)-1-[3-(2-methoxyphenyl)-1-[[2-(trimethylsilyl)ethoxy]methyl]pyrrolo[2,3-b]pyridin-6-yl]urea

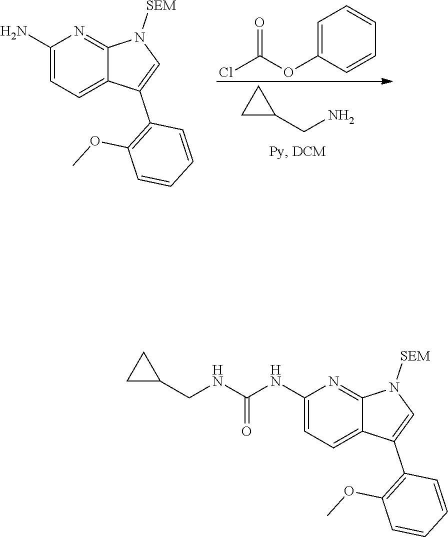

To a solution of 3-(2-methoxyphenyl)-1-[[2-(trimethylsilyl)ethoxy]methyl]pyrrolo[2,3-b]pyridin-6-amine (130.0 mg, 0.35 mmol) in DCM (10.0 mL) was added pyridine (111.3 mg, 1.41 mmol) and phenyl chloroformate (66.1 mg, 0.42 mmol) at room temperature. The resulting mixture was stirred at room temperature for 3 h. The resulting mixture was concentrated under reduced pressure. To the above mixture was added 1-cyclopropylmethanamine (75.1 mg, 1.06 mmol) and pyridine (10.0 mL) at room temperature. The resulting mixture was stirred at 70° C. for another 16 h. After the reaction was completed, the resulting mixture was concentrated under reduced pressure. The residue was purified by flash column chromatography with petroleum ether/ethyl acetate (50/50, v/v) to afford 3-(cyclopropylmethyl)-1-[3-(2-methoxyphenyl)-1-[[2-(trimethylsilyl)ethoxy]methyl]pyrrolo[2,3-b]pyridin-6-yl]urea (160.0 mg, 97%) as a light brown oil. LCMS (ESI, m/z): [M+H]⁺=467.2.

### Step 2: Synthesis of 3-(cyclopropylmethyl)-1-[3-(2-methoxyphenyl)-1H-pyrrolo[2,3-b]pyridin-6-yl]urea (Compound 122)

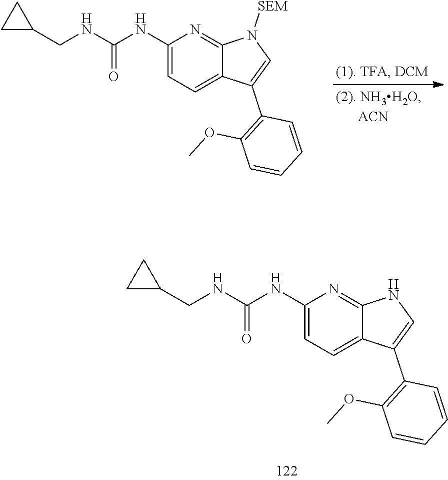 [SMILES: COc1ccccc1-c1cn(COCC[Si](C)(C)C)c2nc(NC(=O)NCC3CC3)ccc12 ; COc1ccccc1-c1c[nH]c2nc(NC(=O)NCC3CC3)ccc12]

To a solution of 3-(cyclopropylmethyl)-1-[3-(2-methoxyphenyl)-1-[[2-(trimethylsilyl)ethoxy]methyl]pyrrolo[2,3-b]pyridin-6-yl]urea (165.0 mg, 0.35 mmol) in CH₂Cl₂ (6.0 mL) was added TFA (6.0 mL) at room temperature. The resulting mixture was stirred at room temperature for 3 h. The resulting mixture was concentrated under reduced pressure. The residue was dissolved in CH₃CN (6.0 mL) and NH₃·H₂O (6.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 45% B to 53% B in 9 min; 254 nm) to afford 3-(cyclopropylmethyl)-1-[3-(2-methoxyphenyl)-1H-pyrrolo[2,3-b]pyridin-6-yl]urea (13.5 mg, 11%) as a light yellow solid. LCMS (ESI, m/z): [M+H]⁺=337.3. ¹H NMR (400 MHz, DMSO-d₆): δ 11.49 (s, 1H), 9.14 (s, 1H), 8.56 (s, 1H), 7.90 (d, J=8.8 Hz, 1H), 7.52-7.46 (m, 2H), 7.28-7.24 (m, 1H), 7.10 (d, J=8.0 Hz, 1H), 7.05-6.99 (m, 2H), 3.81 (s, 3H), 3.15-3.12 (m, 2H), 1.06-0.99 (m, 1H), 0.50-0.45 (m, 2H), 0.24-0.19 (m, 2H).

### Example S123. Compound 123

### Step 1: Synthesis of 3-cyclopropyl-1-[3-(2-methoxyphenyl)-1-[[2-(trimethylsilyl)ethoxy]methyl]pyrrolo[2,3-b]pyridin-6-yl]urea

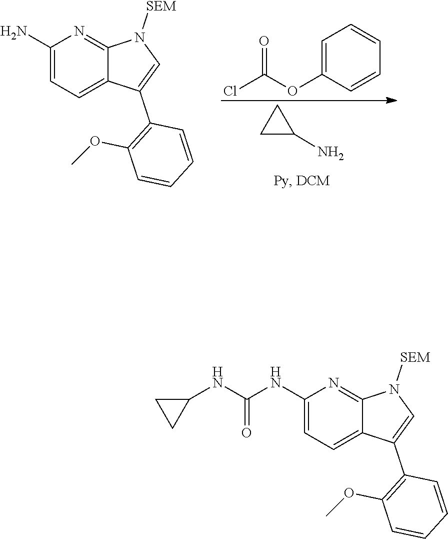

To a solution of 3-(2-methoxyphenyl)-1-[[2-(trimethylsilyl)ethoxy]methyl]pyrrolo[2,3-b]pyridin-6-amine (130.0 mg, 0.35 mmol) in DCM (10.0 mL) was added pyridine (111.3 mg, 1.41 mmol) and phenyl chloroformate (66.1 mg, 0.42 mmol) at room temperature. The resulting mixture was stirred at room temperature for 16 h. The resulting mixture was concentrated under reduced pressure. To the above mixture was added aminocyclopropane (60.3 mg, 1.06 mmol) and pyridine (10.0 mL) at room temperature. The resulting mixture was stirred at 70° C. for another 3 h. After the reaction was completed, the resulting mixture was concentrated under reduced pressure. The residue was purified by flash column chromatography with petroleum ether/ethyl acetate (50/50, v/v) to afford 3-cyclopropyl-1-[3-(2-methoxyphenyl)-1-[[2-(trimethylsilyl)ethoxy]methyl]pyrrolo[2,3-b]pyridin-6-yl]urea (120.0 mg, 75%) as a light brown oil. LCMS (ESI, m/z): [M+H]⁺=453.2.

### Step 2: Synthesis of 3-cyclopropyl-1-[3-(2-methoxyphenyl)-1H-pyrrolo[2,3-b]pyridin-6-yl]urea (Compound 123)

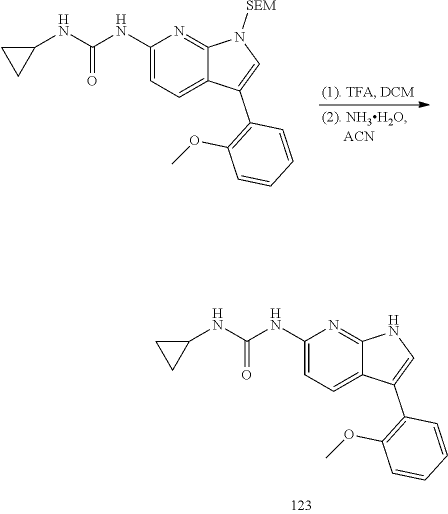 [SMILES: COc1ccccc1-c1cn(COCC[Si](C)(C)C)c2nc(NC(=O)NC3CC3)ccc12 ; COc1ccccc1-c1c[nH]c2nc(NC(=O)NC3CC3)ccc12]

To a solution of 3-cyclopropyl-1-[3-(2-methoxyphenyl)-1-[[2-(trimethylsilyl)ethoxy]methyl]pyrrolo[2,3-b]pyridin-6-yl]urea (120.0 mg, 0.27 mmol) in CH₂Cl₂ (6.0 mL) was added TFA (6.0 mL) at room temperature. The resulting mixture was stirred at room temperature for 16 h. The resulting mixture was concentrated under reduced pressure. The residue was dissolved in CH₃CN (6.0 mL) and NH₃·H₂O (6.0 mL). The resulting mixture was stirred at room temperature for another 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 41% B to 51% B in 8 min; 254 nm) to afford 3-cyclopropyl-1-[3-(2-methoxyphenyl)-1H-pyrrolo[2,3-b]pyridin-6-yl]urea (35.8 mg, 41%) as a white solid. LCMS (ESI, m/z): [M+H]⁺=323.3. ¹H NMR (400 MHz, DMSO-d₆): δ 11.53 (s, 1H), 9.13 (s, 1H), 8.73 (s, 1H), 7.90 (d, J=8.8 Hz, 1H), 7.51-7.46 (m, 2H), 7.28-7.23 (m, 1H), 7.10 (d, J=7.6 Hz, 1H), 7.02-6.98 (m, 2H), 3.80 (s, 3H), 2.68-2.65 (m, 1H), 0.72-0.69 (m, 2H), 0.53-0.50 (m, 2H).

### Example S124. Compound 124

### Step 1: Synthesis of tert-butyl N-(2-[2-[(4-methylbenzenesulfonyl)oxy]ethoxy]ethyl)carbamate

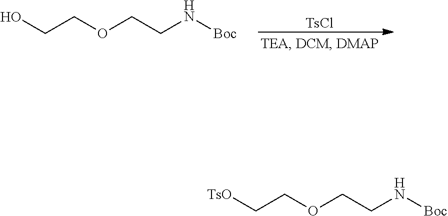 [SMILES: CC(C)(C)OC(=O)NCCOCCO ; "TsCl" ; Cc1ccc(S(=O)(=O)OCCOCCNC(=O)OC(C)(C)C)cc1]

To a solution of tert-butyl N-[2-(2-hydroxyethoxy)ethyl]carbamate (2.0 g, 9.74 mmol) in CH₂Cl₂ (15.0 mL) were added p-toluenesulfonyl chloride (3.7 g, 19.49 mmol), TEA (4.9 g, 48.72 mmol) and DMAP (119.0 mg, 0.97 mmol). The mixture was stirred at room temperature for 16 h. After the reaction was completed, the resulting mixture was concentrated under vacuum. The residue was purified by flash column chromatography with petroleum ether/ethyl acetate (45/55, v/v) to afford tert-butyl N-(2-[2-[(4-methylbenzenesulfonyl)oxy]ethoxy]ethyl)carbamate (2.3 g, 65%) as a yellow solid. LCMS (ESI, m/z): [M+H]⁺=360.1.

### Step 2: Synthesis of tert-butyl N-[2-[2-(dimethylamino)ethoxy]ethyl]carbamate

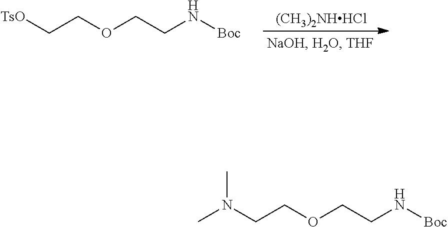

To a solution of tert-butyl N-(2-[2-[(4-methylbenzenesulfonyl)oxy]ethoxy]ethyl)carbamate (2.2 g, 6.12 mmol) in THF/H₂O (20.0 mL/3.0 mL) was added dimethylamine hydrochloride (2.0 g, 24.48 mmol) and NaOH (0.9 g, 24.48 mmol). The mixture was stirred at room temperature for 16 h. After the reaction was completed, the resulting mixture was concentrated under vacuum. The residue was purified by flash column chromatography with CH₂Cl₂/MeOH (95/5, v/v) to afford tert-butyl N-[2-[2-(dimethylamino)ethoxy]ethyl]carbamate (1.2 g, 84%) as a yellow solid. LCMS (ESI, m/z): [M+H]⁺=233.2.

### Step 3: Synthesis of 2-(2-aminoethoxy)-N,N-dimethylethan-1-amine hydrochloride

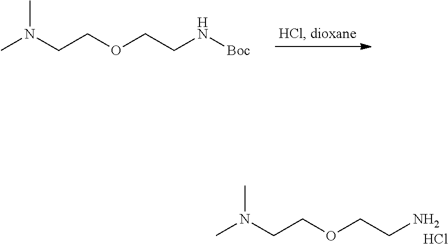

A solution of tert-butyl N-[2-[2-(dimethylamino)ethoxy]ethyl]carbamate (1.2 g, 5.16 mmol) in HCl/1,4-dioxane (10.0 mL, 4 mol/L) was stirred at room temperature for 2 h. The mixture was concentrated under vacuum to afford 2-(2-aminoethoxy)-N,N-dimethylethan-1-amine hydrochloride (700.0 mg, crude) as a yellow solid. LCMS (ESI, m/z): [M+H]⁺=133.1.

### Step 4: Synthesis of 3-[2-[2-(dimethylamino)ethoxy]ethyl]-1-[3-(2-methoxyphenyl)-1-[[2-(trimethylsilyl)ethoxy]methyl]pyrrolo[2,3-b]pyridin-6-yl]urea

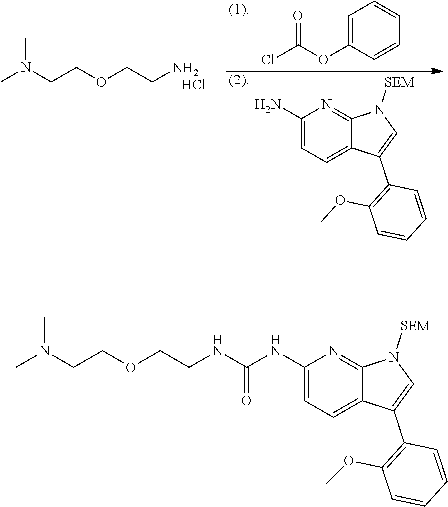

To a solution of 3-(2-methoxyphenyl)-1-[[2-(trimethylsilyl)ethoxy]methyl]pyrrolo[2,3-b]pyridin-6-amine (150.0 mg, 0.41 mmol) in CH₂Cl₂ (3.0 mL) was added pyridine (128.4 mg, 1.62 mmol) and phenyl chloroformate (76.3 mg, 0.49 mmol) at 0° C. The mixture was stirred at room temperature for 2 h. The mixture was concentrated under vacuum. To the above mixture was added pyridine (3.0 mL) and 2-(2-aminoethoxy)-N,N-dimethylethan-1-amine hydrochloride (176.8 mg, crude). The resulting mixture was stirred at 60° C. for another 2 h. After the reaction was completed, the resulting mixture was concentrated under vacuum. The residue was purified by flash column chromatography with CH₂Cl₂/MeOH (96/4, v/v) to afford 3-[2-[2-(dimethylamino)ethoxy]ethyl]-1-[3-(2-methoxyphenyl)-1-[[2-(trimethylsilyl)ethoxy]methyl]pyrrolo[2,3-b]pyridin-6-yl]urea (180.0 mg, 84%) as a yellow solid. LCMS (ESI, m/z): [M+H]⁺=528.3.

### Step 5: Synthesis of 3-[2-[2-(dimethylamino)ethoxy]ethyl]-1-[3-(2-methoxyphenyl)-1H-pyrrolo[2,3-b]pyridin-6-yl]urea (Compound 124)

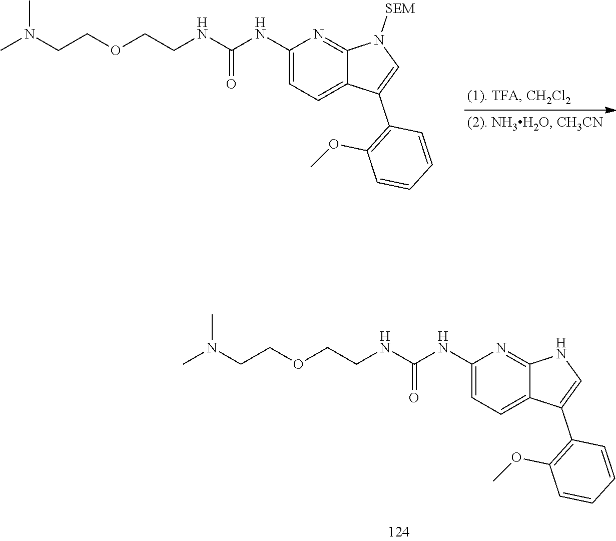 [SMILES: COc1ccccc1-c1cn(COCC[Si](C)(C)C)c2nc(NC(=O)NCCOCCN(C)C)ccc12 ; COc1ccccc1-c1c[nH]c2nc(NC(=O)NCCOCCN(C)C)ccc12]

To a solution of 3-[2-[2-(dimethylamino)ethoxy]ethyl]-1-[3-(2-methoxyphenyl)-1-[[2-(trimethylsilyl)ethoxy]methyl]pyrrolo[2,3-b]pyridin-6-yl]urea (150.0 mg, 0.28 mmol) in CH₂Cl₂ (3.0 mL) was added TFA (3.0 mL). The mixture was stirred at room temperature for 2 h. The mixture was concentrated under vacuum. To the above mixture were added ACN (3.0 mL) and NH₃·H₂O (3.0 mL). The resulting mixture was stirred at room temperature for another 2 h. After the reaction was completed, the resulting mixture was concentrated under vacuum. The residue was purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 39% B to 49% B in 7 min; 254 nm) to afford 3-[2-[2-(dimethylamino)ethoxy]ethyl]-1-[3-(2-methoxyphenyl)-1H-pyrrolo[2,3-b]pyridin-6-yl]urea (41.0 mg, 36%) as a white solid. LCMS (ESI, m/z): [M+H]⁺=398.2. ¹H NMR (300 MHz, DMSO-d₆): δ 11.63 (s, 1H), 9.23 (s, 1H), 8.72 (s, 1H), 7.91 (d, J=8.7 Hz, 1H), 7.53-7.47 (m, 2H), 7.29-7.23 (m, 1H), 7.10 (d, J=7.5 Hz, 1H), 7.04-6.99 (m, 2H), 3.81 (s, 3H), 3.58-3.51 (m, 4H), 3.41-3.34 (m, 2H), 2.52-2.44 (m, 2H), 2.16 (s, 6H).

### Example S125. Compound 125

### Step 1: Synthesis of 3-(6-chloro-1-[[2-(trimethylsilyl)ethoxy]methyl]pyrrolo[2,3-b]pyridin-3-yl)-5-fluoro-2,4-dimethoxypyridine

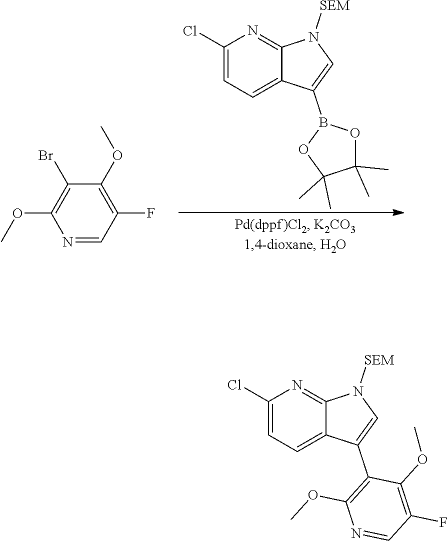 [SMILES: COc1ncc(F)c(OC)c1Br ; CC1(C)OB(c2cn(COCC[Si](C)(C)C)c3nc(Cl)ccc23)OC1(C)C ; COc1ncc(F)c(OC)c1-c1cn(COCC[Si](C)(C)C)c2nc(Cl)ccc12]

To a solution of 3-bromo-5-fluoro-2,4-dimethoxypyridine (345.0 mg, 1.46 mmol) in 1,4-dioxane (7.0 mL) and H₂O (1.4 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (597.5 mg, 1.46 mmol), K₂CO₃ (404.0 mg, 2.92 mmol) and Pd(dppf)Cl₂ (107.0 mg, 0.15 mmol) at room temperature under N₂. The mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (10/1, v/v) to 3-(6-chloro-1-[[2-(trimethylsilyl)ethoxy]methyl]pyrrolo[2,3-b]pyridin-3-yl)-5-fluoro-2,4-dimethoxypyridine (350.0 mg, 57%) as an orange solid. LCMS (ESI, m/z): [M+H]⁺=438.1.

### Step 2: Synthesis of (1R,2R)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide

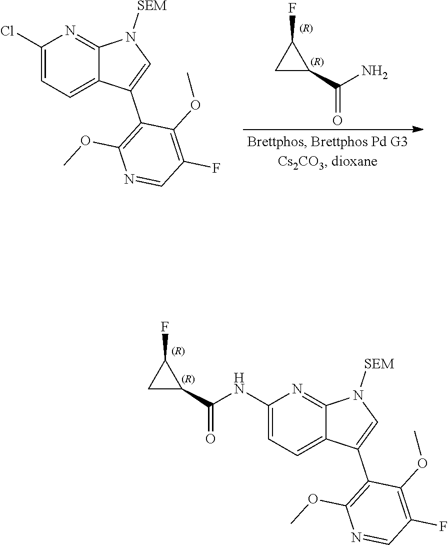

To a solution of 3-(6-chloro-1-[[2-(trimethylsilyl)ethoxy]methyl]pyrrolo[2,3-b]pyridin-3-yl)-5-fluoro-2,4-dimethoxypyridine (300.0 mg, 0.69 mmol) in t-BuOH (10.0 mL) was added (1R,2R)-2-fluorocyclopropane-1-carboxamide (353.1 mg, 3.43 mmol), K₂CO₃ (284.0 mg, 2.06 mmol), XPhos (65.3 mg, 0.14 mmol) and Pd(OAc)₂ (15.4 mg, 0.07 mmol) at room temperature under N₂. The mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford (1R,2R)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide (196.0 mg, 64%) as a yellow solid. LCMS (ESI, m/z): [M+H]⁺=505.2.

### Step 3: Synthesis of (1R,2R)-2-fluoro-N-[3-(5-fluoro-2,4-dimethoxypyridin-3-yl)-1H-pyrrolo[2,3-b]pyridin-6-yl]cyclopropane-1-carboxamide (Compound 125)

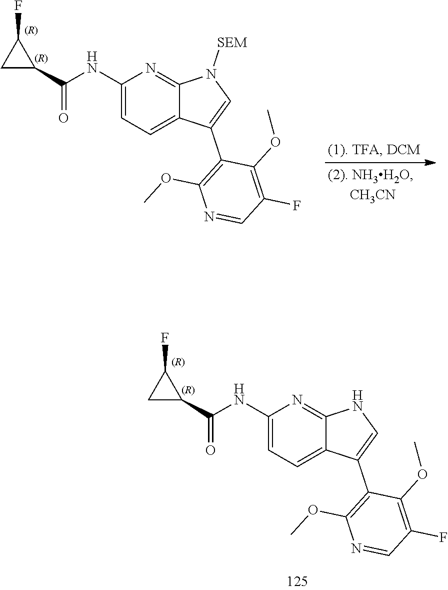

To a solution of (1R,2R)-2-fluoro-N-[3-(5-fluoro-2,4-dimethoxypyridin-3-yl)-1-[[2-(trimethylsilyl)ethoxy]methyl]pyrrolo[2,3-b]pyridin-6-yl]cyclopropane-1-carboxamide (196.0 mg, 0.39 mmol) in CH₂Cl₂ (6.0 mL) was added TFA (3.0 mL) at room temperature. The reaction mixture was stirred at room temperature for 4 h. The mixture was concentrated under vacuum. The residue was dissolved in CH₃CN (10.0 mL) and NH₃·H₂O (10.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 9 min; 254 nm; RT1:8.5 min to afford (1R,2R)-2-fluoro-N-[3-(5-fluoro-2,4-dimethoxypyridin-3-yl)-1H-pyrrolo[2,3-b]pyridin-6-yl]cyclopropane-1-carboxamide (42.0 mg, 29%) as a white solid. LCMS (ESI, m/z): [M+H]⁺=375.0. ¹H NMR (300 MHz, DMSO-d₆): δ 11.70 (d, J=1.8 Hz, 1H), 10.64 (s, 1H), 8.12 (d, J=3.0 Hz, 1H), 7.87 (d, J=8.7 Hz, 1H), 7.71 (d, J=8.7 Hz, 1H), 7.47 (d, J=2.4 Hz, 1H), 5.05-4.80 (m, 1H), 3.81-3.80 (m, 6H), 2.25-2.21 (m, 1H), 1.70-1.60 (m, 1H), 1.19-1.14 (m, 1H).

### Example S126. Compound 126

### Step 1: Synthesis of 6-chloro-3-(2-(2,2,2-trifluoroethoxy)pyridin-3-yl)-1-((2-(trimethylsilyl)ethoxy)methyl)-1H-pyrrolo[2,3-b]pyridine

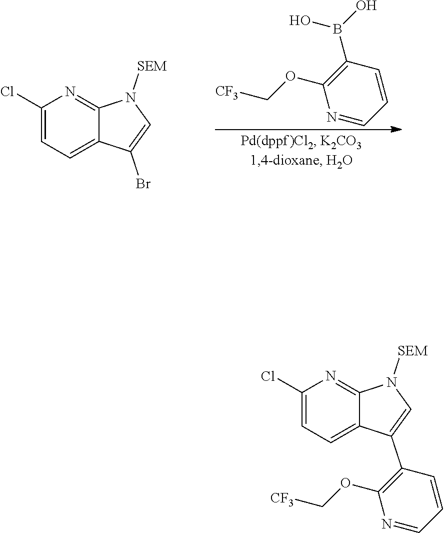

To a solution of 3-bromo-6-chloro-1-((2-(trimethylsilyl)ethoxy)methyl)-1H-pyrrolo[2,3-b]pyridine (500.0 mg, 1.38 mmol) in 1,4-dioxane/H₂O (5.0/1.0 mL) was added (2-(2,2,2-trifluoroethoxy)pyridin-3-yl)boronic acid (305.4 mg, 1.38 mmol), K₂CO₃ (573.1 mg, 4.15 mmol) and Pd(dppf)Cl₂ (101.1 mg, 0.14 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to 6-chloro-3-(2-(2,2,2-trifluoroethoxy)pyridin-3-yl)-1-((2-(trimethylsilyl)ethoxy)methyl)-1H-pyrrolo[2,3-b]pyridine (270.0 mg, 42%) as a yellow oil. LCMS (ESI, m/z): [M+H]⁺=458.1.

### Step 2: Synthesis of (1S,2S)-2-fluoro-N-[3-[2-(2,2,2-trifluoroethoxy)pyridin-3-yl]-1-[[2-(trimethylsilyl)ethoxy]methyl]pyrrolo[2,3-b]pyridin-6-yl]cyclopropane-1-carboxamide

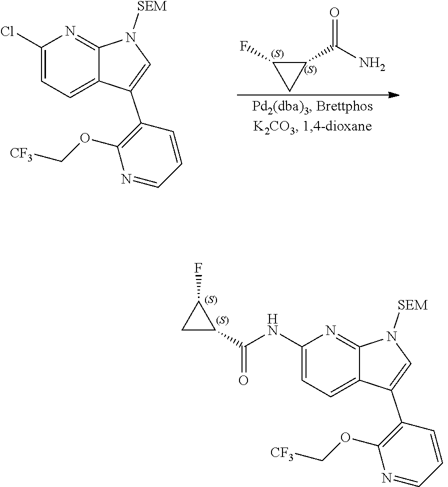

To a solution of 3-(6-chloro-1-[[2-(trimethylsilyl)ethoxy]methyl]pyrrolo[2,3-b]pyridin-3-yl)-2-(2,2,2-trifluoroethoxy)pyridine (126.0 mg, 0.28 mmol) in 1,4-dioxane (5.0 mL) was added (1S,2S)-2-fluorocyclopropane-1-carboxamide (141.8 mg, 1.38 mmol), K₂CO₃ (268.8 mg, 1.95 mmol), BrettPhos (29.5 mg, 0.06 mmol) and Pd₂(dba)₃ (25.2 mg, 0.03 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford (1S,2S)-2-fluoro-N-[3-[2-(2,2,2-trifluoroethoxy)pyridin-3-yl]-1-[[2-(trimethylsilyl)ethoxy]methyl]pyrrolo[2,3-b]pyridin-6-yl]cyclopropane-1-carboxamide (115.0 mg, 79%) as a yellow solid. LCMS (ESI, m/z): [M+H]⁺=525.2.

### Step 3: Synthesis of (1S,2S)-2-fluoro-N-(3-(2-(2,2,2-trifluoroethoxy)pyridin-3-yl)-1H-pyrrolo[2,3-b]pyridin-6-yl)cyclopropane-1-carboxamide (Compound 126)

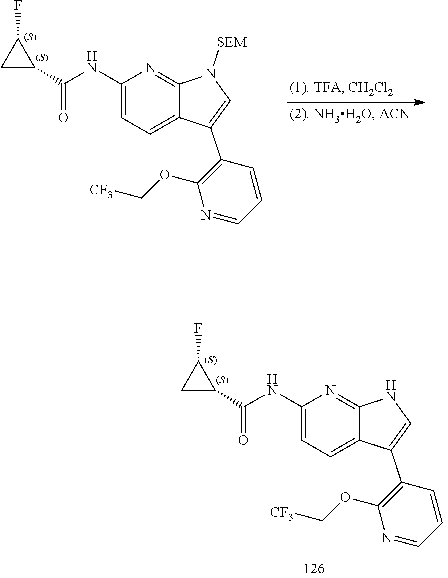

To a solution of (1S,2S)-2-fluoro-N-[3-[2-(2,2,2-trifluoroethoxy)pyridin-3-yl]-1-[[2-(trimethylsilyl)ethoxy]methyl]pyrrolo[2,3-b]pyridin-6-yl]cyclopropane-1-carboxamide (110.0 mg, 0.21 mmol) in CH₂Cl₂ (2.0 mL) was added TFA (2.0 mL) at room temperature under N₂. The resulting mixture was stirred at room temperature for 2 h. The resulting mixture was concentrated under reduced pressure. The residue was dissolved in ACN (2.0 mL) and NH₃·H₂O (2.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 46% B to 53% B in 8 min; 254 nm) to afford (1S,2S)-2-fluoro-N-(3-(2-(2,2,2-trifluoroethoxy)pyridin-3-yl)-1H-pyrrolo[2,3-b]pyridin-6-yl)cyclopropane-1-carboxamide (12.8 mg, 15%) as a white solid. LCMS (ESI, m/z): [M+H]⁺=395.2. ¹H NMR (400 MHz, DMSO-d₆): δ 11.80 (s, 1H), 10.71 (s, 1H), 8.18 (d, J=8.8 Hz, 1H), 8.14-8.10 (m, 2H), 7.94 (d, J=8.4 Hz, 1H), 7.72 (d, J=2.8 Hz, 1H), 7.23-7.20 (m, 1H), 5.10-5.05 (m, 2H), 5.02-4.84 (m, 1H), 2.25-2.19 (m, 1H), 1.69-1.62 (m, 1H), 1.19-1.16 (m, 1H).

### Example S127. Compound 127

### Step 1: Synthesis of 2-bromo-1-cyclopropoxy-3-fluorobenzene

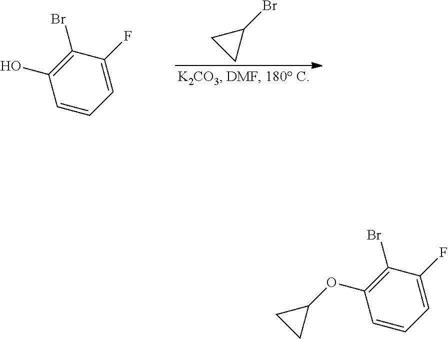

To a solution of 2-bromo-3-fluorophenol (2.5 g, 13.09 mmol) in DMF (50.0 mL) was added bromocyclopropane (9.2 g, 75.89 mmol) and K₂CO₃ (8.7 g, 62.83 mmol) at room temperature. The mixture was stirred at 180° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether to afford 2-bromo-1-cyclopropoxy-3-fluorobenzene (1.4 g, 46%) as a yellow oil.

### Step 2: Synthesis of 6-chloro-3-(2-cyclopropoxy-6-fluorophenyl)-1-[[2-(trimethylsilyl)ethoxy]methyl]pyrrolo[2,3-b]pyridine

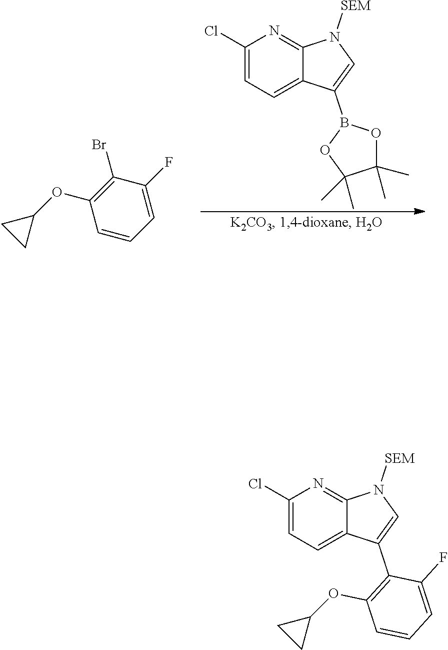 [SMILES: Fc1cccc(OC2CC2)c1Br ; CC1(C)OB(c2cn(COCC[Si](C)(C)C)c3nc(Cl)ccc23)OC1(C)C ; C[Si](C)(C)CCOCn1cc(-c2c(F)cccc2OC2CC2)c2ccc(Cl)nc21]

To a solution of 2-bromo-1-cyclopropoxy-3-fluorobenzene (850.0 mg, 3.68 mmol) in dioxane (17.0 mL) and H₂O (3.4 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (1503.8 mg, 3.68 mmol), K₂CO₃ (1016.8 mg, 7.36 mmol), XPhos (350.7 mg, 0.74 mmol) and XPhos Pd G3 (311.4 mg, 0.37 mmol) at room temperature under N₂. The mixture was stirred at 70° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (10/1, v/v) to afford 6-chloro-3-(2-cyclopropoxy-6-fluorophenyl)-1-[[2-(trimethylsilyl)ethoxy]methyl]pyrrolo[2,3-b]pyridine (453.0 mg, 28%) as a light yellow oil. LCMS (ESI, m/z): [M+H]⁺=433.1.

### Step 3: Synthesis of (1S,2S)—N-[3-(2-cyclopropoxy-6-fluorophenyl)-1-[[2-(trimethylsilyl)ethoxy]methyl]pyrrolo[2,3-b]pyridin-6-yl]-2-fluorocyclopropane-1-carboxamide

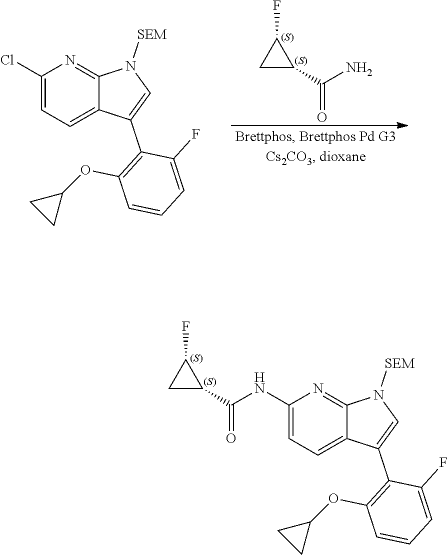

To a solution of 6-chloro-3-(2-cyclopropoxy-6-fluorophenyl)-1-[[2-(trimethylsilyl)ethoxy]methyl]pyrrolo[2,3-b]pyridine (390.0 mg, 0.90 mmol) in t-BuOH (8.0 mL) was added (1S,2S)-2-fluorocyclopropane-1-carboxamide (464.3 mg, 4.50 mmol), K₂CO₃ (311.2 mg, 2.25 mmol), Pd(OAc)₂ (20.2 mg, 0.09 mmol) and XPhos (85.9 mg, 0.18 mmol) at room temperature under N₂. The mixture was stirred at 100° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/EtOAc (2/1, v/v) to afford (1S,2S)—N-[3-(2-cyclopropoxy-6-fluorophenyl)-1-[[2-(trimethylsilyl)ethoxy]methyl]pyrrolo[2,3-b]pyridin-6-yl]-2-fluorocyclopropane-1-carboxamide (76.0 mg, 16%) as a yellow solid. LCMS (ESI, m/z): [M+H]⁺=500.2.

### Step 4: Synthesis of (1S,2S)—N-[3-(2-cyclopropoxy-6-fluorophenyl)-1H-pyrrolo[2,3-b]pyridin-6-yl]-2-fluorocyclopropane-1-carboxamide (Compound 127)

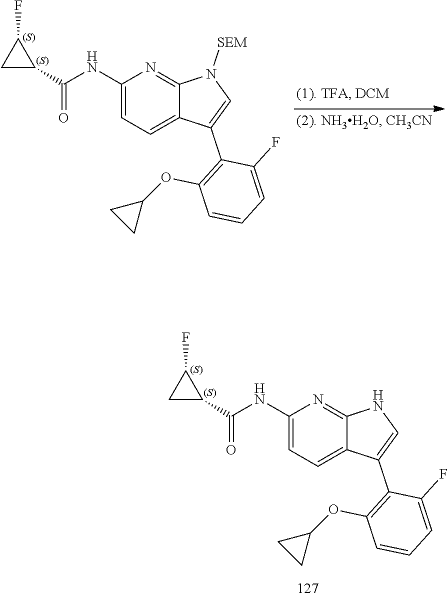

To a solution of (1S,2S)—N-[3-(2-cyclopropoxy-6-fluorophenyl)-1-[[2-(trimethylsilyl)ethoxy]methyl]pyrrolo[2,3-b]pyridin-6-yl]-2-fluorocyclopropane-1-carboxamide (76.0 mg, 0.15 mmol) in CH₂Cl₂ (2.0 mL) was added TFA (2.0 mL) at room temperature. The reaction mixture was stirred at room temperature for 4 h. The mixture was concentrated under vacuum. The residue was dissolved in CH₃CN (4.0 mL) and NH₃·H₂O (4.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 46% B to 56% B in 8 min, 254/220 nm to afford (1S,2S)—N-[3-(2-cyclopropoxy-6-fluorophenyl)-1H-pyrrolo[2,3-b]pyridin-6-yl]-2-fluorocyclopropane-1-carboxamide (8.3 mg, 14%) as a white solid. LCMS (ESI, m/z): [M+H]⁺=370.0. ¹H NMR (300 MHz, DMSO-d₆): δ 11.66 (s, 1H), 10.64 (s, 1H), 7.87 (d, J=8.7 Hz, 1H), 7.67-7.63 (m, 1H), 7.43-7.25 (m, 3H), 6.97-6.93 (m, 1H), 5.06-4.79 (m, 1H), 3.90-3.83 (m, 1H), 2.27-2.19 (m, 1H), 1.72-1.61 (m, 1H), 1.21-1.10 (m, 1H), 0.80-0.76 (m, 2H), 0.65-0.60 (m, 2H).

### Example S128. Compound 128

### Step 1: Synthesis of 6-bromo-7-methoxyimidazo[1,2-a]pyrimidine

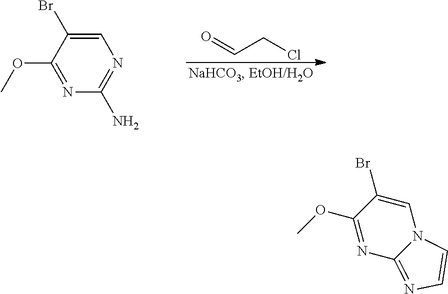

To a solution of 5-bromo-4-methoxypyrimidin-2-amine (500.0 mg, 2.45 mmol) in EtOH/H₂O (10.0 mL/2.5 mL) was added 2-chloroacetaldehyde (1.9 g, 9.80 mmol) and NaHCO₃ (247.1 mg, 2.94 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (84/16, v/v) to afford 6-bromo-7-methoxyimidazo[1,2-a]pyrimidine (80.0 mg, 14%) as a yellow solid. LCMS (ESI, m/z): [M+H]⁺=228.0.

### Step 2: Synthesis of 6-(6-chloro-1-((2-(trimethylsilyl)ethoxy)methyl)-1H-pyrrolo[2,3-b]pyridin-3-yl)-7-methoxyimidazo[1,2-a]pyrimidine

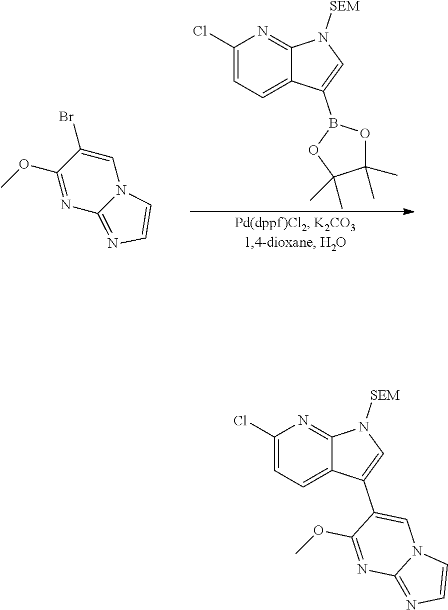 [SMILES: COc1nc2nccn2cc1Br ; CC1(C)OB(c2cn(COCC[Si](C)(C)C)c3nc(Cl)ccc23)OC1(C)C ; COc1nc2nccn2cc1-c1cn(COCC[Si](C)(C)C)c2nc(Cl)ccc12]

To a solution of 6-bromo-7-methoxyimidazo[1,2-a]pyrimidine (140.0 mg, 0.61 mmol) in 1,4-dioxane/H₂O (10.0 mL/2.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (200.5 mg, 0.61 mmol), K₂CO₃ (254.5 mg, 1.84 mmol) and Pd(dppf)Cl₂ (44.9 mg, 0.06 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/91, v/v) to afford 6-(6-chloro-1-((2-(trimethylsilyl)ethoxy)methyl)-1H-pyrrolo[2,3-b]pyridin-3-yl)-7-methoxyimidazo[1,2-a]pyrimidine (200.0 mg, 76%) as a brown solid. LCMS (ESI, m/z): [M+H]⁺=430.1.

### Step 3: Synthesis of (1S,2S)-2-fluoro-N-(3-(7-methoxyimidazo[1,2-a]pyrimidin-6-yl)-1-((2-(trimethylsilyl)ethoxy)methyl)-1H-pyrrolo[2,3-b]pyridin-6-yl)cyclopropane-1-carboxamide

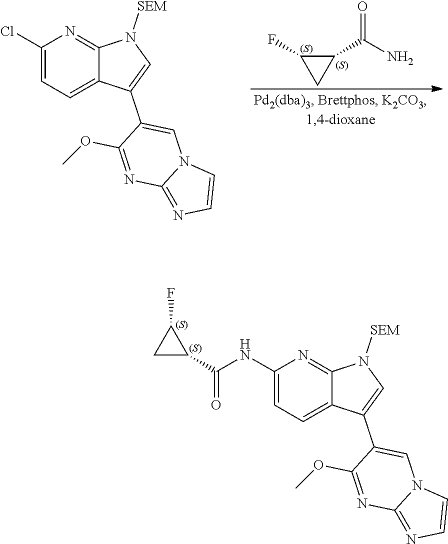

To a solution of 6-(6-chloro-1-((2-(trimethylsilyl)ethoxy)methyl)-1H-pyrrolo[2,3-b]pyridin-3-yl)-7-methoxyimidazo[1,2-a]pyrimidine (160.0 mg, 0.37 mmol) in 1,4-dioxane (5.0 mL) was added (1S,2S)-2-fluorocyclopropane-1-carboxamide (191.8 mg, 1.86 mmol), K₂CO₃ (154.3 mg, 1.12 mmol), BrettPhos (40.0 mg, 0.07 mmol) and Pd₂(dba)₃ (34.1 mg, 0.04 mmol) at room temperature under N₂. The resulting mixture was stirred at 100° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (30/70, v/v) to afford (1S,2S)-2-fluoro-N-(3-(7-methoxyimidazo[1,2-a]pyrimidin-6-yl)-1-((2-(trimethylsilyl)ethoxy)methyl)-1H-pyrrolo[2,3-b]pyridin-6-yl)cyclopropane-1-carboxamide (238.0 mg, 90%) as a brown solid. LCMS (ESI, m/z): [M+H]⁺=497.2.

### Step 4: Synthesis of (1S,2S)-2-fluoro-N-(3-(7-methoxyimidazo[1,2-a]pyrimidin-6-yl)-1H-pyrrolo[2,3-b]pyridin-6-yl)cyclopropane-1-carboxamide (Compound 128)

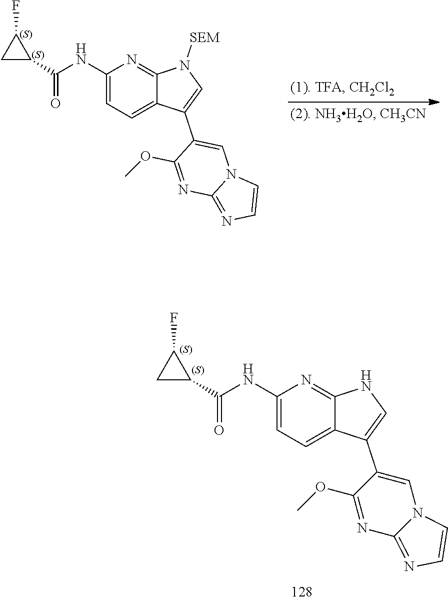

To a solution of (1S,2S)-2-fluoro-N-(3-(7-methoxyimidazo[1,2-a]pyrimidin-6-yl)-1-((2-(trimethylsilyl)ethoxy)methyl)-1H-pyrrolo[2,3-b]pyridin-6-yl)cyclopropane-1-carboxamide (238.0 mg, 0.48 mmol) in CH₂Cl₂ (10.0 mL) was added TFA (10.0 mL) at room temperature. The resulting mixture was stirred at room temperature for 4 h. The resulting mixture was concentrated under reduced pressure. To the residue in CH₃CN (10.0 mL) was added NH₃·H₂O (10.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 21% B to 31% B in 9 min, 254 nm) to afford (1S,2S)-2-fluoro-N-(3-(7-methoxyimidazo[1,2-a]pyrimidin-6-yl)-1H-pyrrolo[2,3-b]pyridin-6-yl)cyclopropane-1-carboxamide (4.2 mg, 1%) as a white solid. LCMS (ESI, m/z): [M+H]⁺=367.2. ¹H NMR (300 MHz, DMSO-d₆): δ 11.80 (s, 1H), 10.70 (s, 1H), 9.03 (s, 1H), 8.19 (d, J=8.7 Hz, 1H), 7.95 (d, J=8.7 Hz, 1H), 7.72 (d, J=1.5 Hz, 2H), 7.43 (d, J=1.5 Hz, 1H), 5.06-4.80 (m, 1H), 4.03 (s, 3H), 2.27-2.22 (m, 1H), 1.73-1.60 (m, 1H), 1.24-1.11 (m, 1H).

### Example S129. Compound 129

### Step 1: Synthesis of tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate

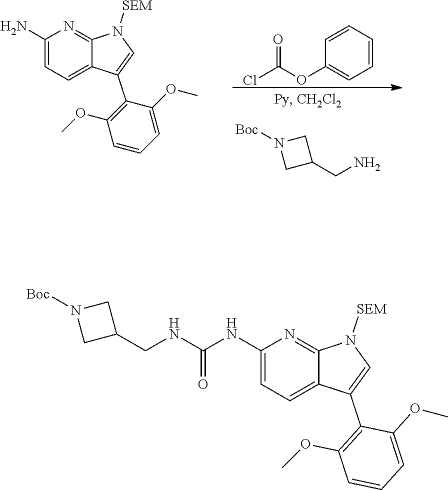

To a solution of 3-(2,6-dimethoxyphenyl)-1-((2-(trimethylsilyl)ethoxy)methyl)-1H-pyrrolo[2,3-b]pyridin-6-amine (100.0 mg, 0.25 mmol) in CH₂Cl₂ (5.0 mL) was added phenyl chloroformate (58.8 mg, 0.38 mmol) and pyridine (80.0 mg, 1.01 mmol) at 0° C. under N₂. The resulting mixture was stirred at 0° C. for 1 h. The resulting mixture was concentrated under reduced pressure. To the above mixture in pyridine (5.0 mL) was added tert-butyl 3-(aminomethyl)azetidine-1-carboxylate (186.5 mg, 1.00 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (90/10, v/v) to afford tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate (97.0 mg, 63%) as a light yellow oil. LCMS (ESI, m/z): [M+H]⁺=612.3.

### Step 2: Synthesis of 1-(azetidin-3-ylmethyl)-3-(3-(2,6-dimethoxyphenyl)-1H-pyrrolo[2,3-b]pyridin-6-yl)urea (Compound 129)

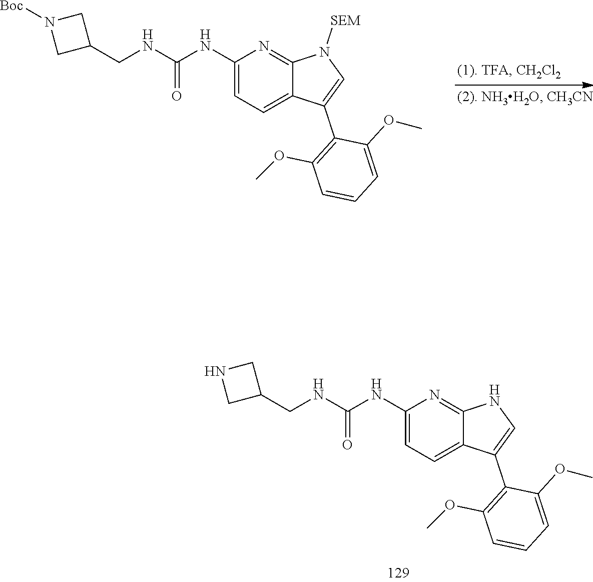

To a solution of tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate (120.0 mg, 0.20 mmol) in CH₂Cl₂ (1.0 mL) was added TFA (1.0 mL) at room temperature. The resulting mixture was stirred at room temperature for 4 h. The resulting mixture was concentrated under reduced pressure. To the residue in CH₃CN (1.0 mL) was added NH₃·H₂O (1.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 28% B to 35% B in 8 min; 254 nm) to afford 1-(azetidin-3-ylmethyl)-3-(3-(2,6-dimethoxyphenyl)-1H-pyrrolo[2,3-b]pyridin-6-yl)urea (13.0 mg, 17.4%) as a white solid. LCMS (ESI, m/z): [M+H]⁺=382.4. ¹H NMR (400 MHz, DMSO-d₆+D₂O): δ 7.46 (d, J=8.4 Hz, 1H), 7.27-7.21 (m, 1H), 7.17 (s, 1H), 6.87 (d, J=8.4 Hz, 1H), 6.73 (d, J=8.4 Hz, 2H), 3.86-3.74 (m, 2H), 3.68-3.57 (m, 2H), 3.49-3.39 (m, 2H), 2.95-2.92 (m, 1H).

### Example S130. Compound 130

### Step 1: Synthesis of tert-butyl N-[3-([[3-(2,6-dimethoxyphenyl)-1-[[2-(trimethylsilyl)ethoxy]methyl]pyrrolo[2,3-b]pyridin-6-yl]carbamoyl]amino)propyl]-N-methylcarbamate

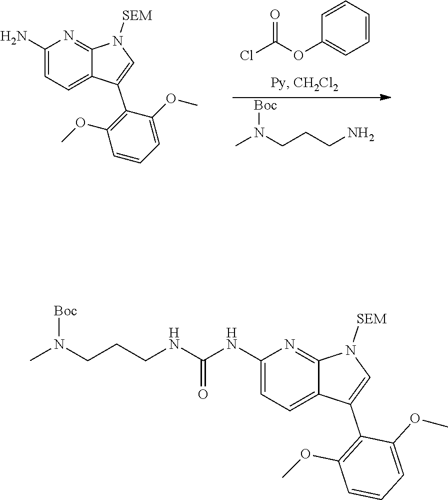

To a solution of 3-(2,6-dimethoxyphenyl)-1-[[2-(trimethylsilyl)ethoxy]methyl]pyrrolo[2,3-b]pyridin-6-amine (130.0 mg, 0.33 mmol) in DCM (5.0 mL) was added pyridine (104.23 mg, 1.30 mmol) and phenyl chloroformate (61.1 mg, 0.39 mmol) at room temperature. The resulting mixture was stirred at room temperature for 3 h. The resulting mixture was concentrated under reduced pressure. To the above mixture was added tert-butyl N-(3-aminopropyl)-N-methylcarbamate (183.8 mg, 0.98 mmol) and pyridine (5.0 mL) at room temperature. The resulting mixture was stirred at 60° C. for another 2 h. After the reaction was completed, the resulting mixture was concentrated under reduced pressure. The residue was purified by flash column chromatography with petroleum ether/ethyl acetate (90/10, v/v) to afford tert-butyl N-[3-([[3-(2,6-dimethoxyphenyl)-1-[[2-(trimethylsilyl)ethoxy]methyl]pyrrolo[2,3-b]pyridin-6-yl]carbamoyl]amino)propyl]-N-methylcarbamate (180.0 mg, 90%) as a yellow oil. LCMS (ESI, m/z): [M+H]⁺=614.3.

### Step 2: Synthesis of 1-[3-(2,6-dimethoxyphenyl)-1H-pyrrolo[2,3-b]pyridin-6-yl]-3-[3-(methylamino)propyl]urea (Compound 130)

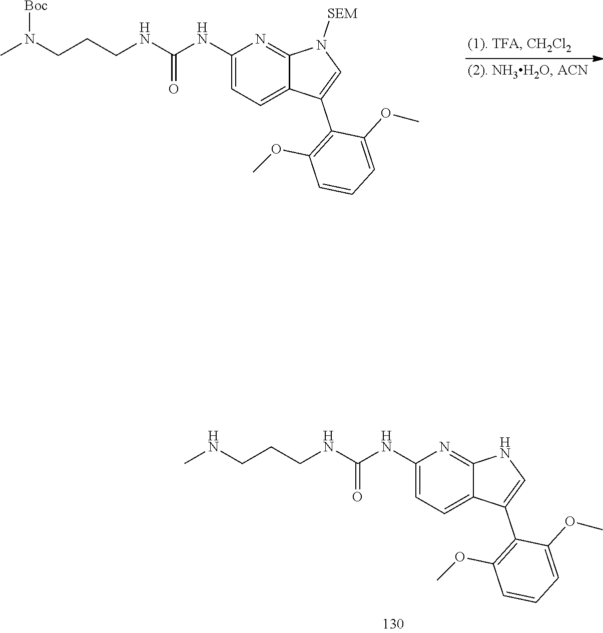

To a solution of tert-butyl N-[3-([[3-(2,6-dimethoxyphenyl)-1-[[2-(trimethylsilyl)ethoxy]methyl]pyrrolo[2,3-b]pyridin-6-yl]carbamoyl]amino)propyl]-N-methylcarbamate (160.0 mg, 0.26 mmol) in CH₂Cl₂ (3.0 mL) was added TFA (3.0 mL) at room temperature. The resulting mixture was stirred at room temperature for 3 h. The resulting mixture was concentrated under reduced pressure. The residue was dissolved in CH₃CN (3.0 mL) and NH₃·H₂O (3.0 mL). The resulting mixture was stirred at room temperature for another 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 15% B to 45% B in 9 min; 254 nm) to afford 1-[3-(2,6-dimethoxyphenyl)-1H-pyrrolo[2,3-b]pyridin-6-yl]-3-[3-(methylamino)propyl]urea (29.9 mg, 29%) as a white solid. LCMS (ESI, m/z): [M+H]⁺=384.3. ¹H NMR (300 MHz, DMSO-d₆): δ 11.39 (s, 1H), 9.13 (s, 1H), 8.67 (s, 1H), 7.47 (d, J=8.4 Hz, 1H), 7.29-7.24 (m, 1H), 7.17 (s, 1H), 6.90 (d, J=8.4 Hz, 1H), 6.74 (d, J=8.4 Hz, 2H), 3.68 (s, 6H), 3.34-3.24 (m, 3H), 2.58-2.50 (m, 2H), 2.30 (s, 3H), 1.71-1.62 (m, 2H).

### Example S131. Compound 131

### Step 1: Synthesis of 1-[3-(2,6-dimethoxyphenyl)-1-[[2-(trimethylsilyl)ethoxy]methyl]pyrrolo[2,3-b]pyridin-6-yl]-3-[3-(dimethylamino)propyl]urea

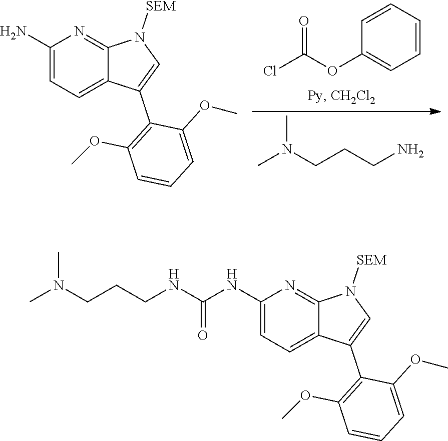

To a solution of 3-(2,6-dimethoxyphenyl)-1-[[2-(trimethylsilyl)ethoxy]methyl]pyrrolo[2,3-b]pyridin-6-amine (130.0 mg, 0.33 mmol) in DCM (6.0 mL) was added pyridine (102.9 mg, 1.30 mmol) and phenyl chloroformate (61.1 mg, 0.39 mmol) at room temperature. The resulting mixture was stirred at room temperature for 3 h. The resulting mixture was concentrated under reduced pressure. To the above mixture was added dimethylaminopropylamine (99.7 mg, 0.98 mmol) and pyridine (6.0 mL) at room temperature. The resulting mixture was stirred at 60° C. for another 16 h. After the reaction was completed, the resulting mixture was concentrated under reduced pressure. The residue was purified by flash column chromatography with petroleum ether/ethyl acetate (9/1, v/v) to afford 1-[3-(2,6-dimethoxyphenyl)-1-[[2-(trimethylsilyl)ethoxy]methyl]pyrrolo[2,3-b]pyridin-6-yl]-3-[3-(dimethylamino)propyl]urea (166.0 mg, 96%) as a yellow oil. LCMS (ESI, m/z): [M+H]⁺=528.3.

### Step 2: Synthesis of 1-[3-(2,6-dimethoxyphenyl)-1H-pyrrolo[2,3-b]pyridin-6-yl]-3-[3-(dimethylamino)propyl]urea (Compound 131)

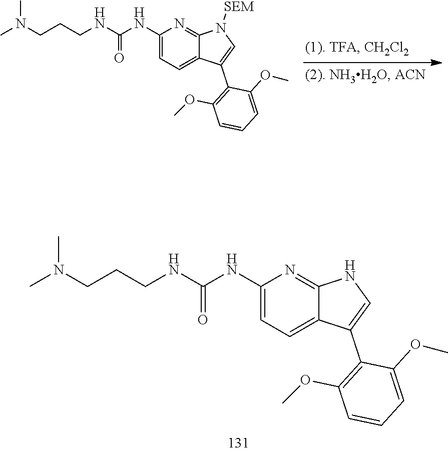

To a solution of 1-[3-(2,6-dimethoxyphenyl)-1-[[2-(trimethylsilyl)ethoxy]methyl]pyrrolo[2,3-b]pyridin-6-yl]-3-[3-(dimethylamino)propyl]urea (172.0 mg, 0.33 mmol) in CH₂Cl₂ (6.0 mL) was added TFA (6.0 mL) at room temperature. The resulting mixture was stirred at room temperature for 3 h. The resulting mixture was concentrated under reduced pressure. To the residue in CH₃CN (6.0 mL) was added NH₃·H₂O (6.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 27% B to 57% B in 7 min; 254 nm) to afford 1-[3-(2,6-dimethoxyphenyl)-1H-pyrrolo[2,3-b]pyridin-6-yl]-3-[3-(dimethylamino)propyl]urea (25.8 mg, 19%) as a light yellow solid. LCMS (ESI, m/z): [M+H]⁺=398.3. ¹H NMR (400 MHz, DMSO-d₆): δ 11.35 (s, 1H), 9.11 (s, 1H), 8.60 (s, 1H), 7.46 (d, J=8.4 Hz, 1H), 7.28-7.24 (m, 1H), 7.15 (d, J=2.4 Hz, 1H), 6.91 (d, J=8.4 Hz, 1H), 6.74 (d, J=8.4 Hz, 2H), 3.68 (s, 6H), 3.26-3.21 (m, 2H), 2.29-2.25 (m, 2H), 2.14 (s, 6H), 1.68-1.61 (m, 2H).

### Example S132. Compound 132

### Step 1: Synthesis of 1-[3-(2,6-dimethoxyphenyl)-1-[[2-(trimethylsilyl)ethoxy]methyl]pyrrolo[2,3-b]pyridin-6-yl]-3-[2-(dimethylamino)ethyl]urea

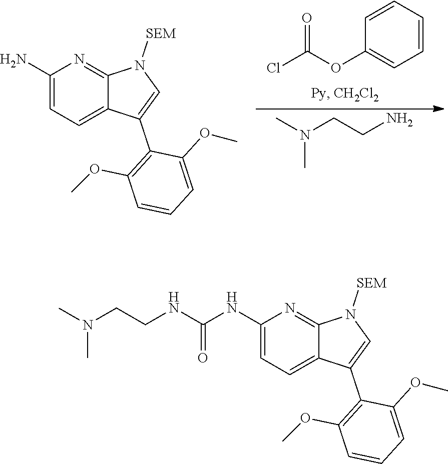

To a solution of 3-(2,6-dimethoxyphenyl)-1-[[2-(trimethylsilyl)ethoxy]methyl]pyrrolo[2,3-b]pyridin-6-amine (130.0 mg, 0.33 mmol) in DCM (13.0 mL) was added pyridine (104.2 mg, 1.30 mmol) and phenyl chloroformate (61.1 mg, 0.39 mmol) at room temperature. The resulting mixture was stirred at room temperature for 16 h. The resulting mixture was concentrated under reduced pressure. To the residue in pyridine (10.0 mL) was added (2-aminoethyl)dimethylamine (143.4 mg, 1.63 mmol) at room temperature. The resulting mixture was stirred at 60° C. for 4 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford 1-[3-(2,6-dimethoxyphenyl)-1-[[2-(trimethylsilyl)ethoxy]methyl]pyrrolo[2,3-b]pyridin-6-yl]-3-[2-(dimethylamino)ethyl]urea (127.0 mg, 76%) as a yellow oil. LCMS (ESI, m/z): [M+H]⁺=514.3.

### Step 2: Synthesis of 1-[3-(2,6-dimethoxyphenyl)-1H-pyrrolo[2,3-b]pyridin-6-yl]-3-[2-(dimethylamino)ethyl]urea (Compound 132)

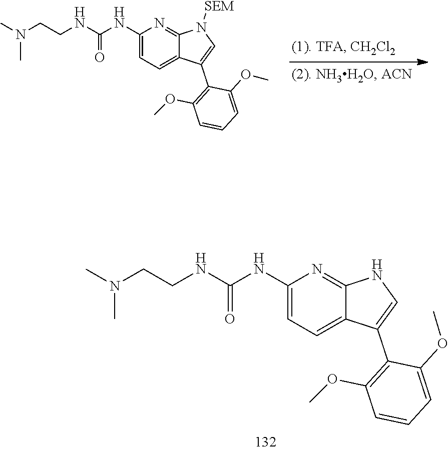

To a solution of 1-[3-(2,6-dimethoxyphenyl)-1-[[2-(trimethylsilyl)ethoxy]methyl]pyrrolo[2,3-b]pyridin-6-yl]-3-[2-(dimethylamino)ethyl]urea (107.0 mg, 0.21 mmol) in CH₂Cl₂ (8.0 mL) was added TFA (8.0 mL) at room temperature. The resulting mixture was stirred at room temperature for 2 h. The resulting mixture was concentrated under reduced pressure. To the residue in CH₃CN (8.0 mL) was added NH₃·H₂O (8.0 mL) at room temperature. The resulting mixture was stirred at room temperature for 16 h. After the reaction was completed, the resulting mixture was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 35% B to 43% B in 8 min; 254 nm) to afford 1-[3-(2,6-dimethoxyphenyl)-1H-pyrrolo[2,3-b]pyridin-6-yl]-3-[2-(dimethylamino)ethyl]urea (26.4 mg, 33%) as a light green solid. LCMS (ESI, m/z): [M+H]⁺=384.2. ¹H NMR (400 MHz, DMSO-d₆): δ 11.28 (d, J=1.6 Hz, 1H), 9.11 (s, 1H), 8.46 (s, 1H), 7.46 (d, J=8.4 Hz, 1H), 7.28-7.24 (m, 1H), 7.15 (d, J=2.4 Hz, 1H), 6.97 (d, J=8.4 Hz, 1H), 6.73 (d, J=8.4 Hz, 2H), 3.68 (s, 6H), 3.33-3.28 (m, 2H), 2.41-2.38 (m, 2H), 2.21 (s, 6H).

### Example S133. Compound 133

### Step 1: Synthesis of 4-bromo-2-fluoro-3-methoxyaniline

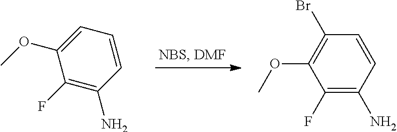

To a solution of 2-fluoro-3-methoxyaniline (2.0 g, 14.17 mmol) in DMF (20.0 mL) was added NBS (2.5 g, 14.17 mmol) at room temperature. The resulting mixture was stirred at room temperature for 2 h. After the reaction was completed, the reaction mixture was quenched with Na₂SO₃ (aq.). The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 4-bromo-2-fluoro-3-methoxyaniline (2.1 g, 67%) as a white solid. LCMS (ESI, m/z): [M+H]⁺=220.0.

### Step 2: Synthesis of 6-bromo-4-fluoro-5-methoxy-1,3-benzothiazol-2-amine

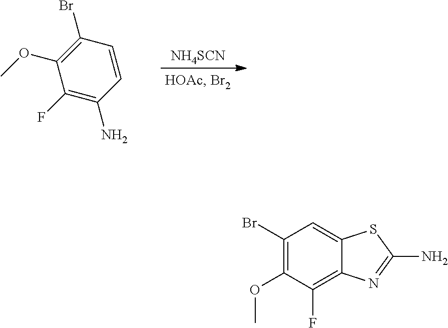

To a mixture of 4-bromo-2-fluoro-3-methoxyaniline (1.0 g, 4.54 mmol) and NH₄SCN (380.5 mg, 5.00 mmol) in acetic acid (40.0 mL) was added dropwise Br₂ (871.5 mg, 5.45 mmol) at room temperature. The resulting mixture was stirred at room temperature for 16 h. After the reaction was completed, the mixture was basified to pH=8 with saturated Na₂CO₃ (aq.). The resulting mixture was diluted with H₂O and extracted with ethyl acetate. The combined organic layer was washed with brine, dried over anhydrous sodium sulfate and filtered. The filtrate was concentrated under reduced pressure to afford 6-bromo-4-fluoro-5-methoxy-1,3-benzothiazol-2-amine (750.0 mg, 59%) as an off-white solid. LCMS (ESI, m/z): [M+H]⁺=276.9.

### Step 3: Synthesis of 6-bromo-4-fluoro-5-methoxy-1,3-benzothiazole

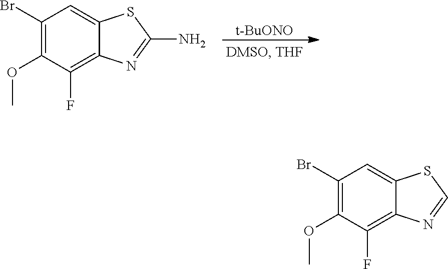

To a mixture of 6-bromo-4-fluoro-5-methoxy-1,3-benzothiazol-2-amine (380.0 mg, 1.37 mmol) and 2-methyl-2-propylnitrite (212.1 mg, 2.05 mmol) in THE (10.0 mL) was added dropwise DMSO (10.7 mg, 0.14 mmol). The resulting mixture was stirred at room temperature for 16 h. The resulting mixture was concentrated under reduced pressure. The residue was purified by flash column chromatography with petroleum ether/ethyl acetate (10/1, v/v) to afford 6-bromo-4-fluoro-5-methoxy-1,3-benzothiazole (200.0 mg, 55%) as a yellow solid. LCMS (ESI, m/z): [M+H]⁺=261.9.

### Step 4: Synthesis of 6-(6-chloro-1-[[2-(trimethylsilyl)ethoxy]methyl]pyrrolo[2,3-b]pyridin-3-yl)-4-fluoro-5-methoxy-1,3-benzothiazole

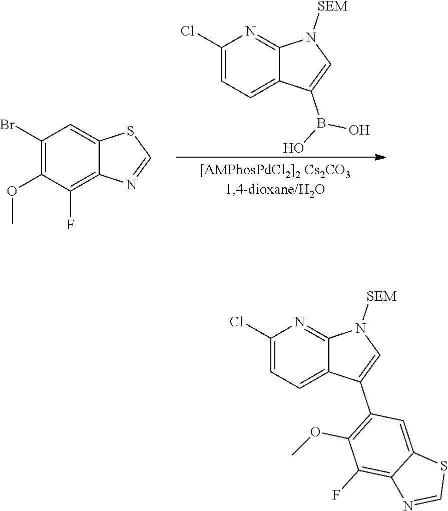 [SMILES: COc1c(Br)cc2scnc2c1F ; C[Si](C)(C)CCOCn1cc(B(O)O)c2ccc(Cl)nc21 ; COc1c(-c2cn(COCC[Si](C)(C)C)c3nc(Cl)ccc23)cc2scnc2c1F]

To a mixture of 6-chloro-1-[[2-(trimethylsilyl)ethoxy]methyl]pyrrolo[2,3-b]pyridin-3-ylboronic acid (548.4 mg, 1.68 mmol) and 6-bromo-4-fluoro-5-methoxy-1,3-benzothiazole (400.0 mg, 1.52 mmol) in dioxane (10.0 mL) were added [AMPhosPdCl₂]₂ (216.1 mg, 0.31 mmol), Cs₂CO₃ (1491.7 mg, 4.58 mmol) and H₂O (1.0 mL) at room temperature. The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-(6-chloro-1-[[2-(trimethylsilyl)ethoxy]methyl]pyrrolo[2,3-b]pyridin-3-yl)-4-fluoro-5-methoxy-1,3-benzothiazole (400.0 mg, 56%) as a yellow solid. LCMS (ESI, m/z): [M+H]⁺=464.1.

### Step 5: Synthesis of (1S,2S)-2-fluoro-N-[3-(4-fluoro-5-methoxy-1,3-benzothiazol-6-yl)-1-[[2-(trimethylsilyl)ethoxy]methyl]pyrrolo[2,3-b]pyridin-6-yl]cyclopropane-1-carboxamide

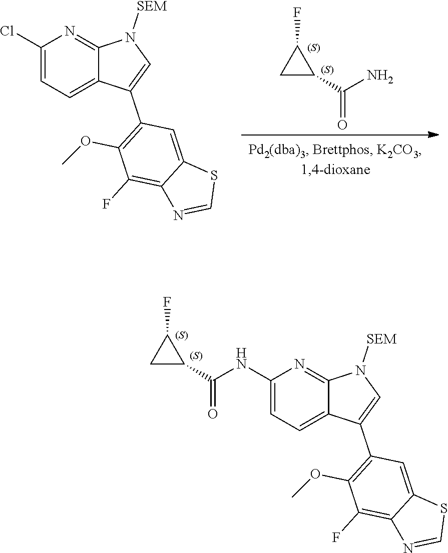

To a mixture of 6-(6-chloro-1-[[2-(trimethylsilyl)ethoxy]methyl]pyrrolo[2,3-b]pyridin-3-yl)-4-fluoro-5-methoxy-1,3-benzothiazole (300.0 mg, 0.68 mmol) and (1S,2S)-2-fluorocyclopropane-1-carboxamide (333.3 mg, 3.23 mmol) in 1,4-dioxane (5.0 mL) was added Pd₂(dba)₃ (118.4 mg, 0.13 mmol), BrettPhos (138.8 mg, 0.26 mmol) and K₂CO₃ (268.0 mg, 1.94 mmol). The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-[3-(4-fluoro-5-methoxy-1,3-benzothiazol-6-yl)-1-[[2-(trimethylsilyl)ethoxy]methyl]pyrrolo[2,3-b]pyridin-6-yl]cyclopropane-1-carboxamide (50.0 mg, 14%) as a yellow solid. LCMS (ESI, m/z): [M+H]⁺=531.2.

### Step 6: Synthesis of (1S,2S)-2-fluoro-N-[3-(4-fluoro-5-methoxy-1,3-benzothiazol-6-yl)-1H-pyrrolo[2,3-b]pyridin-6-yl]cyclopropane-1-carboxamide (Compound 133)

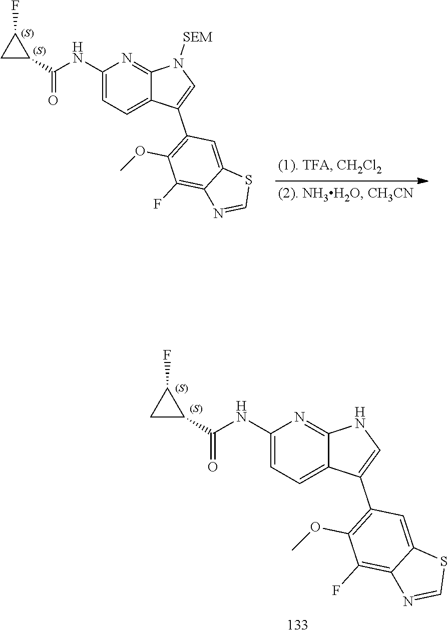

To a solution of (1S,2S)-2-fluoro-N-[3-(4-fluoro-5-methoxy-1,3-benzothiazol-6-yl)-1-[[2-(trimethylsilyl)ethoxy]methyl]pyrrolo[2,3-b]pyridin-6-yl]cyclopropane-1-carboxamide (45.0 mg, 0.09 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 26% B to 35% B in 7 min; 254 nm) to afford (1S,2S)-2-fluoro-N-[3-(4-fluoro-5-methoxy-1,3-benzothiazol-6-yl)-1H-pyrrolo[2,3-b]pyridin-6-yl]cyclopropane-1-carboxamide (11.6 mg, 34%) as a white solid. LCMS (ESI, m/z): [M+H]⁺=401.1. ¹H NMR (300 MHz, DMSO-d₆): δ 11.80 (s, 1H), 10.68 (s, 1H), 9.36 (s, 1H), 8.19-8.17 (m, 2H), 7.93 (d, J=8.4 Hz, 1H), 7.75 (d, J=2.7 Hz, 1H), 5.05-4.78 (m, 1H), 3.74 (s, 3H), 2.25-2.18 (m, 1H), 1.88-1.58 (m, 1H), 1.22-1.07 (m, 1H).

### Example S134. Compound 134

### Step 1: Synthesis of (E)-N′-(5-bromo-6-methoxypyridin-2-yl)-N,N-dimethylformimidamide

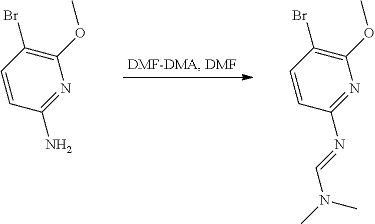

To a solution of 5-bromo-6-methoxypyridin-2-amine (1.5 g, 7.42 mmol) in DMF (10.0 mL) was added DMF-DMA (972.0 mg, 8.17 mmol). The resulting mixture was stirred at 70° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (E)-N′-(5-bromo-6-methoxypyridin-2-yl)-N,N-dimethylformimidamide (1.5 g, 79%) as an off-white solid. LCMS (ESI, m/z): [M+H]⁺=258.0.

### Step 2: Synthesis of (E)-N′-(5-bromo-6-methoxypyridin-2-yl)-N-hydroxymethanimidamide

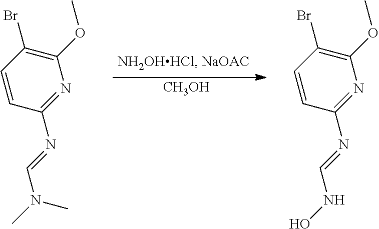

To a solution of (E)-N′-(5-bromo-6-methoxypyridin-2-yl)-N,N-dimethylformimidamide (1.5 g, 5.81 mmol) in methanol (10.0 mL) was added hydroxylamine hydrochloride (1.5 g, 21.44 mmol) and NaOAc (1.5 g, 18.85 mmol). The resulting mixture was stirred at 70° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (E)-N′-(5-bromo-6-methoxypyridin-2-yl)-N-hydroxymethanimidamide (1.4 g, 98%) as an off-white solid. LCMS (ESI, m/z): [M+H]⁺=246.0.

### Step 3: Synthesis of 6-bromo-5-methoxy-[1,2,4]triazolo[1,5-a]pyridine

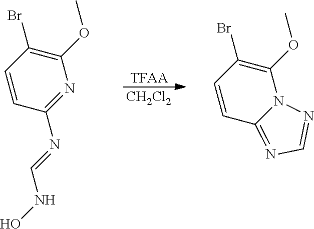

To a solution of (E)-N′-(5-bromo-6-methoxypyridin-2-yl)-N-hydroxymethanimidamide (1.4 g, 5.69 mmol) in DCM (10.0 mL) was added TFAA (1.3 g, 6.27 mmol) at room temperature. The resulting mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-bromo-5-methoxy-[1,2,4]triazolo[1,5-a]pyridine (600.0 mg, 46%) as a white solid. LCMS (ESI, m/z): [M+H]⁺=228.0.

### Step 4: Synthesis of 6-chloro-3-[5-methoxy-[1,2,4]triazolo[1,5-a]pyridin-6-yl]-1-[[2-(trimethylsilyl)ethoxy]methyl]pyrrolo[2,3-b]pyridine

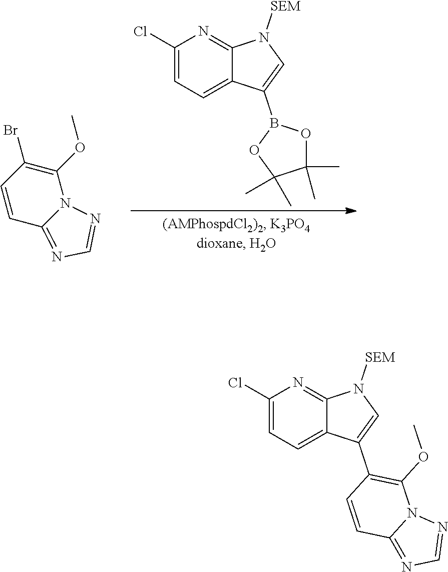 [SMILES: COc1c(Br)ccc2ncnn12 ; CC1(C)OB(c2cn(COCC[Si](C)(C)C)c3nc(Cl)ccc23)OC1(C)C ; COc1c(-c2cn(COCC[Si](C)(C)C)c3nc(Cl)ccc23)ccc2ncnn12]

To a solution of 6-bromo-5-methoxy-[1,2,4]triazolo[1,5-a]pyridine (300.0 mg, 1.36 mmol) in dioxane (5.0 mL) and H₂O (0.5 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (645.5 mg, 1.59 mmol), (AMPhospdCl₂)₂ (93.1 mg, 0.12 mmol) and K₃PO₄ (837.7 mg, 3.94 mmol). The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-chloro-3-[5-methoxy-[1,2,4]triazolo[1,5-a]pyridin-6-yl]-1-[[2-(trimethylsilyl)ethoxy]methyl]pyrrolo[2,3-b]pyridine (200.0 mg, 35%) as a white solid. LCMS (ESI, m/z): [M+H]⁺=430.1.

### Step 5: Synthesis of (1S,2S)-2-fluoro-N-(3-(5-methoxy-[1,2,4]triazolo[1,5-a]pyridin-6-yl)-1-((2-(trimethylsilyl)ethoxy)methyl)-1H-pyrrolo[2,3-b]pyridin-6-yl)cyclopropane-1-carboxamide

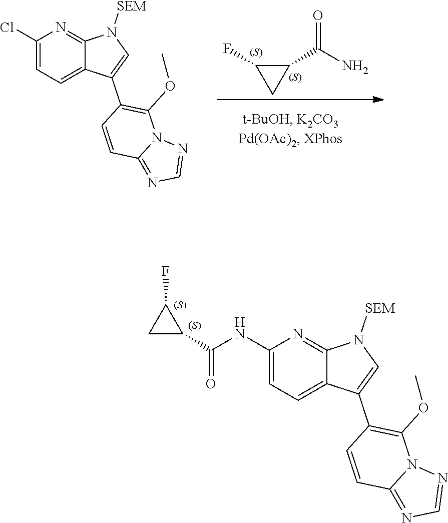

To a solution of 6-chloro-3-[5-methoxy-[1,2,4]triazolo[1,5-a]pyridin-6-yl]-1-[[2-(trimethylsilyl)ethoxy]methyl]pyrrolo[2,3-b]pyridine (200.0 mg, 0.45 mmol) in t-BuOH (5.0 mL) was added (1S,2S)-2-fluorocyclopropane-1-carboxamide (479.4 mg, 4.61 mmol), Pd(OAc)₂ (20.9 mg, 0.09 mmol), XPhos (22.7 mg, 0.05 mmol) and K₂CO₃ (192.8 mg, 1.39 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-(3-(5-methoxy-[1,2,4]triazolo[1,5-a]pyridin-6-yl)-1-((2-(trimethylsilyl)ethoxy)methyl)-1H-pyrrolo[2,3-b]pyridin-6-yl)cyclopropane-1-carboxamide (70.0 mg, 30%) as a white solid. LCMS (ESI, m/z): [M+H]⁺=497.2.

### Step 6: Synthesis of (1S,2S)-2-fluoro-N-(3-[5-methoxy-[1,2,4]triazolo[1,5-a]pyridin-6-yl]-1H-pyrrolo[2,3-b]pyridin-6-yl)cyclopropane-1-carboxamide (Compound 134)

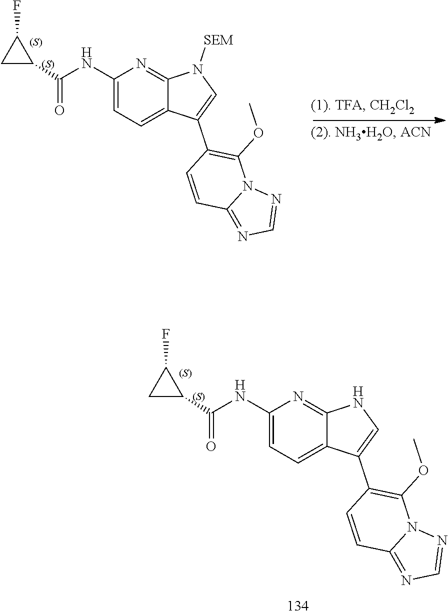

To a solution of (1S,2S)-2-fluoro-N-(3-[5-methoxy-[1,2,4]triazolo[1,5-a]pyridin-6-yl]-1-[[2-(trimethylsilyl)ethoxy]methyl]pyrrolo[2,3-b]pyridin-6-yl)cyclopropane-1-carboxamide (70.0 mg, 0.14 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 24% B to 32% B in 7 min; 254 nm) to afford (1S,2S)-2-fluoro-N-(3-[5-methoxy-[1,2,4]triazolo[1,5-a]pyridin-6-yl]-1H-pyrrolo[2,3-b]pyridin-6-yl)cyclopropane-1-carboxamide (1.2 mg, 2%) LCMS (ESI, m/z): [M+H]⁺=367.3. ¹H NMR (300 MHz, DMSO-d₆): δ 11.87 (s, 1H), 10.70 (s, 1H), 8.55 (s, 1H), 8.09 (d, J=8.4 Hz, 1H), 7.99-7.93 (m, 2H), 7.73-7.70 (m, 2H), 5.05-4.82 (m, 1H), 4.03 (s, 3H), 2.30-2.19 (m, 1H), 1.70-1.60 (m, 1H), 1.23-1.13 (m, 1H).

### Example S135. Compound 135

### Step 1: Synthesis of tert-butyl 4-[2-([[3-(2,6-dimethoxyphenyl)-1-[[2-(trimethylsilyl)ethoxy]methyl]pyrrolo[2,3-b]pyridin-6-yl]carbamoyl]amino)ethyl]piperazine-1-carboxylate

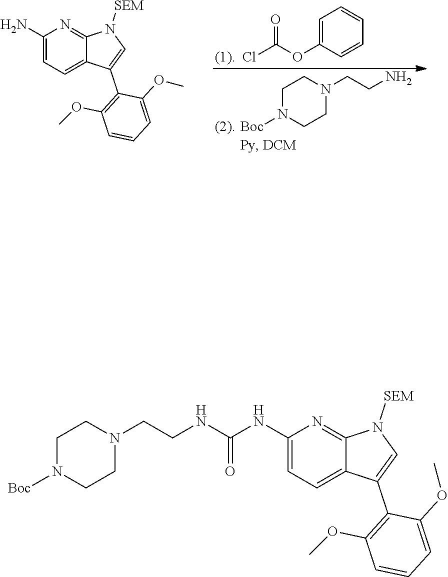

To a solution of 3-(2,6-dimethoxyphenyl)-1-[[2-(trimethylsilyl)ethoxy]methyl]pyrrolo[2,3-b]pyridin-6-amine (110.0 mg, 0.28 mmol) in CH₂Cl₂ (3.0 mL) was added pyridine (87.1 mg, 1.10 mmol) and phenyl chloroformate (51.7 mg, 0.33 mmol) at 0° C. The mixture was stirred at room temperature for 2 h. The mixture was concentrated under vacuum. To the above mixture was added pyridine (2.0 mL) and tert-butyl 4-(2-aminoethyl)piperazine-1-carboxylate (189.4 mg, 0.83 mmol). The resulting mixture was stirred at 60° C. for 2 h. After the reaction was completed, the resulting mixture was concentrated under vacuum. The residue was purified by flash column chromatography with CH₂Cl₂/MeOH (95/5, v/v) to afford tert-butyl 4-[2-([[3-(2,6-dimethoxyphenyl)-1-[[2-(trimethylsilyl)ethoxy]methyl]pyrrolo[2,3-b]pyridin-6-yl]carbamoyl]amino)ethyl]piperazine-1-carboxylate (110.0 mg, 61%) as a yellow solid. LCMS (ESI, m/z): [M+H]⁺=655.4.

### Step 2: Synthesis of 3-[3-(2,6-dimethoxyphenyl)-1H-pyrrolo[2,3-b]pyridin-6-yl]-1-[2-(piperazin-1-yl)ethyl]urea (Compound 135)

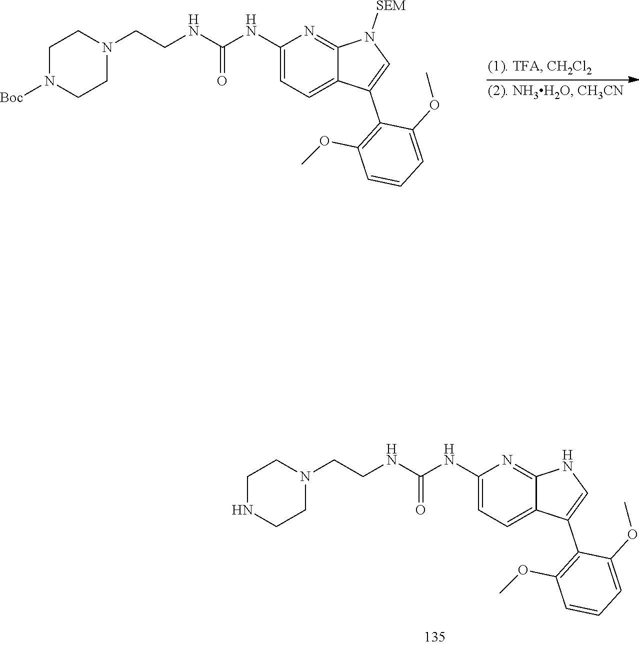 [SMILES: COc1cccc(OC)c1-c1cn(COCC[Si](C)(C)C)c2nc(NC(=O)NCCN3CCN(C(=O)OC(C)(C)C)CC3)ccc12 ; COc1cccc(OC)c1-c1c[nH]c2nc(NC(=O)NCCN3CCNCC3)ccc12]

To a solution of tert-butyl 4-[2-([[3-(2,6-dimethoxyphenyl)-1-[[2-(trimethylsilyl)ethoxy]methyl]pyrrolo[2,3-b]pyridin-6-yl]carbamoyl]amino)ethyl]piperazine-1-carboxylate (120.0 mg, 0.18 mmol) in CH₂Cl₂ (3.0 mL) was added TFA (3.0 mL). The mixture was stirred at room temperature for 4 h. The mixture was concentrated under vacuum. To the above residue were added ACN (3.0 mL) and NH₃·H₂O (3.0 mL). The resulting mixture was stirred at room temperature for 3 h. After the reaction was completed, the resulting mixture was concentrated under vacuum. The residue was purified by Prep-HPLC with the following conditions (Column: Xselect CSH OBD Column, 30×150 mm, 5 um; Mobile Phase A: Water (10 mmol/L NH₄HCO₃), Mobile Phase B: ACN; Flow rate: 60 mL/min; Gradient: 32% B to 42% B in 8 min; 254 nm) to afford 3-[3-(2,6-dimethoxyphenyl)-1H-pyrrolo[2,3-b]pyridin-6-yl]-1-[2-(piperazin-1-yl)ethyl]urea (24.6 mg, 31%) as a white solid. LCMS (ESI, m/z): [M+H]⁺=425.3. ¹H NMR (300 MHz, DMSO-d₆): δ 11.30 (s, 1H), 9.15 (s, 1H), 8.41 (s, 1H), 7.46 (d, J=8.7 Hz, 1H), 7.29-7.24 (m, 1H), 7.17 (d, J=2.1 Hz, 1H), 6.99 (d, J=8.4 Hz, 1H), 6.74 (d, J=8.4 Hz, 2H), 3.69 (s, 6H), 2.73-2.70 (m, 4H), 2.46-2.37 (m, 6H).

### Example S136. Compound 136

### Step 1: Synthesis of 3-(2-cyclopropoxyphenyl)-1-[[2-(trimethylsilyl)ethoxy]methyl]pyrazolo[3,4-b]pyridin-6-amine

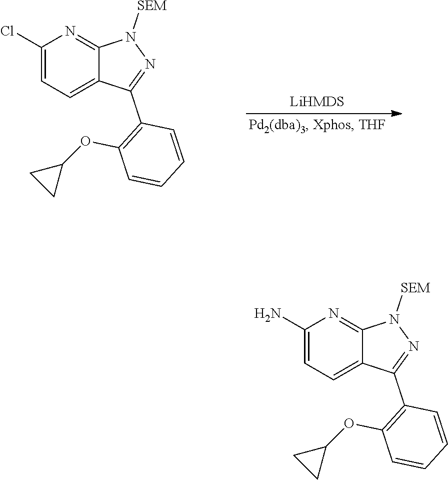 [SMILES: C[Si](C)(C)CCOCn1nc(-c2ccccc2OC2CC2)c2ccc(Cl)nc21 ; C[Si](C)(C)CCOCn1nc(-c2ccccc2OC2CC2)c2ccc(N)nc21]

To a solution of 6-chloro-3-(2-cyclopropoxyphenyl)-1-[[2-(trimethylsilyl)ethoxy]methyl]pyrazolo[3,4-b]pyridine (320.0 mg, 0.77 mmol) in THE (5.0 mL) was added X-Phos (73.3 mg, 0.15 mmol), Pd₂(dba)₃ (70.4 mg, 0.08 mmol) and LiHMDS (1.2 mL, 1 mol/L) at room temperature. The resulting mixture was stirred at 60° C. for 1 h. After the reaction was completed, the reaction was quenched with water. The resulting mixture was concentrated under vacuum. The residue was purified by flash column chromatography with CH₂Cl₂/MeOH (93/7, v/v) to afford 3-(2-cyclopropoxyphenyl)-1-[[2-(trimethylsilyl)ethoxy]methyl]pyrazolo[3,4-b]pyridin-6-amine (120.0 mg, 39%) as a yellow solid. LCMS (ESI, m/z): [M+H]⁺=397.2.

### Step 2: Synthesis of tert-butyl 4-[2-([[3-(2-cyclopropoxyphenyl)-1-[[2-(trimethylsilyl)ethoxy]methyl]pyrazolo[3,4-b]pyridin-6-yl]carbamoyl]amino)ethyl]piperazine-1-carboxylate

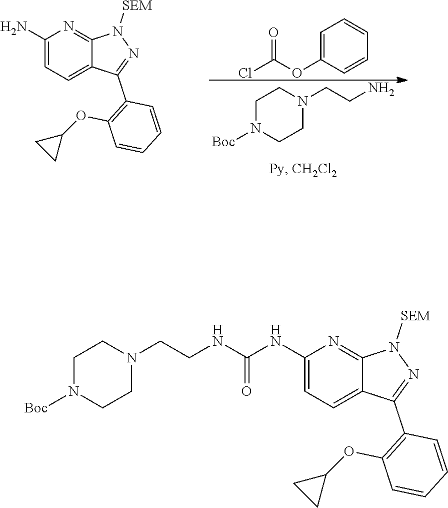

To a solution of 3-(2-cyclopropoxyphenyl)-1-[[2-(trimethylsilyl)ethoxy]methyl]pyrazolo[3,4-b]pyridin-6-amine (100.0 mg, 0.25 mmol) in CH₂Cl₂ (3.0 mL) was added pyridine (79.8 mg, 1.01 mmol) and phenyl chloroformate (47.4 mg, 0.30 mmol) at 0° C. The mixture was stirred at room temperature for 2 h. The mixture was concentrated under vacuum. To the above residue was added pyridine (2.0 mL) and tert-butyl 4-(2-aminoethyl)piperazine-1-carboxylate (173.5 mg, 0.76 mmol). The resulting mixture was stirred at 60° C. for 2 h. After the reaction was completed, the resulting mixture was concentrated under vacuum. The residue was purified by flash column chromatography with CH₂Cl₂/MeOH (94/6, v/v) to afford tert-butyl 4-[2-([[3-(2-cyclopropoxyphenyl)-1-[[2-(trimethylsilyl)ethoxy]methyl]pyrazolo[3,4-b]pyridin-6-yl]carbamoyl]amino)ethyl]piperazine-1-carboxylate (100.0 mg, 60%) as a yellow solid. LCMS (ESI, m/z): [M+H]⁺=652.4.

### Step 3: Synthesis of 3-[3-(2-cyclopropoxyphenyl)-1H-pyrazolo[3,4-b]pyridin-6-yl]-1-[2-(piperazin-1-yl)ethyl]urea (Compound 136)

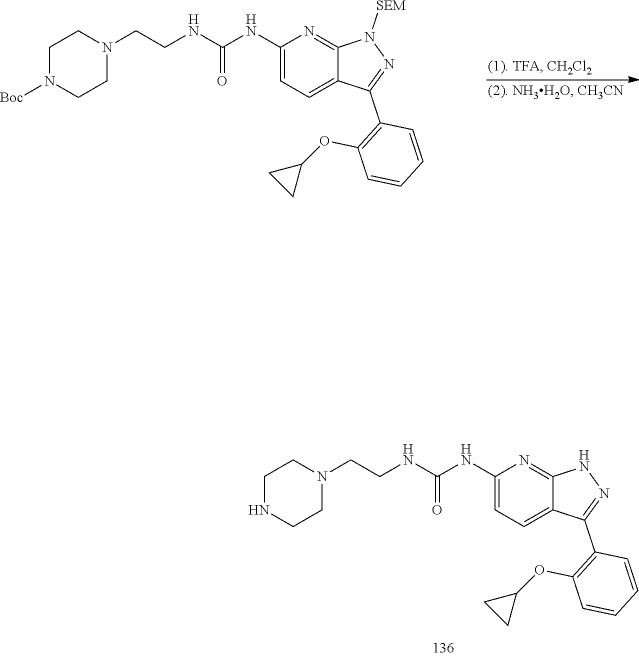 [SMILES: CC(C)(C)OC(=O)N1CCN(CCNC(=O)Nc2ccc3c(-c4ccccc4OC4CC4)nn(COCC[Si](C)(C)C)c3n2)CC1 ; O=C(NCCN1CCNCC1)Nc1ccc2c(-c3ccccc3OC3CC3)n[nH]c2n1]

To a solution of tert-butyl 4-[2-([[3-(2-cyclopropoxyphenyl)-1-[[2-(trimethylsilyl)ethoxy]methyl]pyrazolo[3,4-b]pyridin-6-yl]carbamoyl]amino)ethyl]piperazine-1-carboxylate (80.0 mg, 0.12 mmol) in CH₂Cl₂ (3.0 mL) was added TFA (3.0 mL). The mixture was stirred at room temperature for 3 h. The mixture was concentrated under vacuum. To the above residue were added ACN (3.0 mL) and NH₃·H₂O (3.0 mL). The resulting mixture was stirred at room temperature for another 3 h. After the reaction was completed, the resulting mixture was concentrated under vacuum. The residue was purified by Prep-HPLC with the following conditions (Column: Xselect CSH OBD Column, 19×250 mm, 5 um; Mobile Phase A: Water (10 mmol/L NH₄HCO₃), Mobile Phase B: ACN; Flow rate: 60 mL/min; Gradient: 47% B to 68% B in 9 min; 254 nm) to afford 3-[3-(2-cyclopropoxyphenyl)-1H-pyrazolo[3,4-b]pyridin-6-yl]-1-[2-(piperazin-1-yl)ethyl]urea (14.0 mg, 27%) as a white solid. LCMS (ESI, m/z): [M+H]⁺=422.3. ¹H NMR (300 MHz, DMSO-d₆): δ 13.30 (s, 1H), 9.61 (s, 1H), 8.43-8.39 (m, 1H), 7.90 (d, J=8.7 Hz, 1H), 7.58 (d, J=1.2 Hz, 1H), 7.49-7.41 (m, 2H), 7.16-7.06 (m, 2H), 3.93-3.87 (m, 1H), 3.37-3.23 (s, 3H), 2.78-2.75 (m, 4H), 2.47-2.39 (m, 6H), 0.83-0.73 (m, 2H), 0.68-0.63 (m, 2H).

### Example S137. Compound 137

### Step 1: Synthesis of tert-butyl N-[3-(2,4-dimethoxypyridin-3-yl)-1-[[2-(trimethylsilyl)ethoxy]methyl]pyrrolo[2,3-b]pyridin-6-yl]carbamate

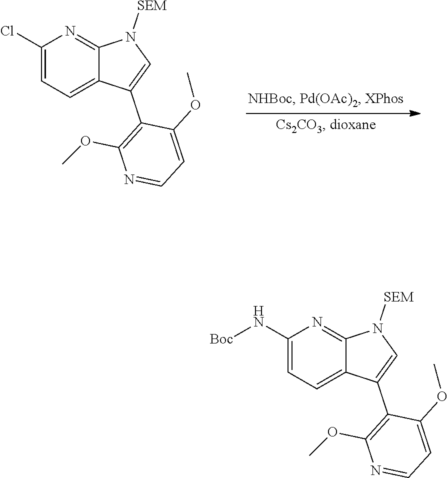 [SMILES: COc1ccnc(OC)c1-c1cn(COCC[Si](C)(C)C)c2nc(Cl)ccc12 ; COc1ccnc(OC)c1-c1cn(COCC[Si](C)(C)C)c2nc(NC(=O)OC(C)(C)C)ccc12]

To a mixture of 3-(6-chloro-1-[[2-(trimethylsilyl)ethoxy]methyl]pyrrolo[2,3-b]pyridin-3-yl)-2,4-dimethoxypyridine (600.0 mg, 1.43 mmol), Cs₂CO₃ (1.4 g, 4.28 mmol) and BocNH₂ (836.8 mg, 7.14 mmol) in dioxane (5.0 mL) were added Pd(OAc)₂ (64.1 mg, 0.28 mmol) and X-Phos (272.4 mg, 0.57 mmol). The resulting mixture was stirred at 80° C. for 16 h under N₂. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-[3-(2,4-dimethoxypyridin-3-yl)-1-[[2-(trimethylsilyl)ethoxy]methyl]pyrrolo[2,3-b]pyridin-6-yl]carbamate (550.0 mg, 77%) as a white solid. LCMS (ESI, m/z): [M+H]⁺=501.2.

### Step 2: Synthesis of 3-(2,4-dimethoxypyridin-3-yl)-1-[[2-(trimethylsilyl)ethoxy]methyl]pyrrolo[2,3-b]pyridin-6-amine

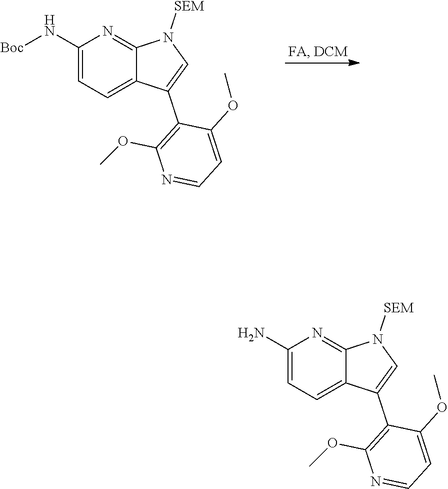 [SMILES: COc1ccnc(OC)c1-c1cn(COCC[Si](C)(C)C)c2nc(NC(=O)OC(C)(C)C)ccc12 ; COc1ccnc(OC)c1-c1cn(COCC[Si](C)(C)C)c2nc(N)ccc12]

A mixture of tert-butyl N-[3-(2,4-dimethoxypyridin-3-yl)-1-[[2-(trimethylsilyl)ethoxy]methyl]pyrrolo[2,3-b]pyridin-6-yl]carbamate (250.0 mg, 0.5 mmol) and formic acid (10.0 mL) in DCM (20.0 mL) was stirred at room temperature for 16 h. After the reaction was completed, the resulting mixture was diluted with H₂O. The pH value of the mixture was adjusted to 8 with saturated NaHCO₃ solution and then extracted with ethyl acetate. The combined organic layer was washed with brine, dried over anhydrous sodium sulfate and filtered. The filtrate was concentrated under reduced pressure to afford 3-(2,4-dimethoxypyridin-3-yl)-1-[[2-(trimethylsilyl)ethoxy]methyl]pyrrolo[2,3-b]pyridin-6-amine (200.0 mg, crude) as a yellow oil. LCMS (ESI, m/z): [M+H]⁺=401.2.

### Step 3: Synthesis of tert-butyl 4-[2-([[3-(2,4-dimethoxypyridin-3-yl)-1-[[2-(trimethylsilyl)ethoxy]methyl]pyrrolo[2,3-b]pyridin-6-yl]carbamoyl]amino)ethyl]piperazine-1-carboxylate

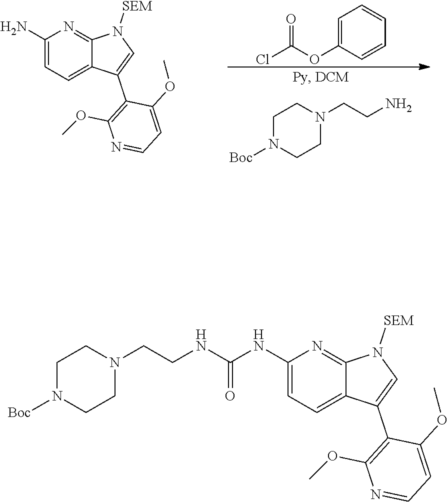 [SMILES: COc1ccnc(OC)c1-c1cn(COCC[Si](C)(C)C)c2nc(N)ccc12 ; CC(C)(C)OC(=O)N1CCN(CCN)CC1 ; O=C(Cl)Oc1ccccc1 ; COc1ccnc(OC)c1-c1cn(COCC[Si](C)(C)C)c2nc(NC(=O)NCCN3CCN(C(=O)OC(C)(C)C)CC3)ccc12]

To a mixture of 3-(2,4-dimethoxypyridin-3-yl)-1-[[2-(trimethylsilyl)ethoxy]methyl]pyrrolo[2,3-b]pyridin-6-amine (100.0 mg, 0.25 mmol) and pyridine (79.9 mg, 1.0 mmol) in DCM (10.0 mL) was added phenyl chloroformate (46.9 mg, 0.30 mmol). The resulting mixture was stirred at room temperature for 3 h. The resulting mixture was concentrated under reduced pressure. To the residue in pyridine (5.0 mL) was added tert-butyl 4-(2-aminoethyl)piperazine-1-carboxylate (343.5 mg, 1.50 mmol) at room temperature. The resulting mixture was stirred at 60° C. for another 16 h. After the reaction was completed, the mixture was concentrated under vacuum. The residue was purified by flash column chromatography with petroleum ether/ethyl acetate (1/1, v/v) to afford tert-butyl 4-[2-([[3-(2,4-dimethoxypyridin-3-yl)-1-[[2-(trimethylsilyl)ethoxy]methyl]pyrrolo[2,3-b]pyridin-6-yl]carbamoyl]amino)ethyl]piperazine-1-carboxylate (100.0 mg, 61%) as a yellow solid. LCMS (ESI, m/z): [M+H]⁺=656.4.

### Step 4: Synthesis of 3-[3-(2,4-dimethoxypyridin-3-yl)-1H-pyrrolo[2,3-b]pyridin-6-yl]-1-[2-(piperazin-1-yl)ethyl]urea (Compound 137)

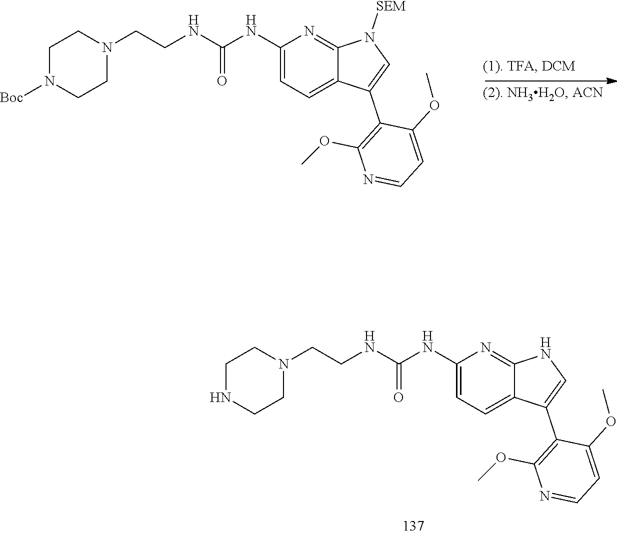 [SMILES: COc1ccnc(OC)c1-c1cn(COCC[Si](C)(C)C)c2nc(NC(=O)NCCN3CCN(C(=O)OC(C)(C)C)CC3)ccc12 ; COc1ccnc(OC)c1-c1c[nH]c2nc(NC(=O)NCCN3CCNCC3)ccc12]

To a solution of tert-butyl 4-[2-([[3-(2,4-dimethoxypyridin-3-yl)-1-[[2-(trimethylsilyl)ethoxy]methyl]pyrrolo[2,3-b]pyridin-6-yl]carbamoyl]amino)ethyl]piperazine-1-carboxylate (170.0 mg, 0.26 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 24% B to 32% B in 7 min; 254 nm) to afford 3-[3-(2,4-dimethoxypyridin-3-yl)-1H-pyrrolo[2,3-b]pyridin-6-yl]-1-[2-(piperazin-1-yl)ethyl]urea (13.2 mg, 11%) as a white solid. LCMS (ESI, m/z): [M+H]⁺=426.2. ¹H NMR (300 MHz, CDCl₃) δ 9.74 (s, 1H), 9.58 (s, 1H), 8.09 (d, J=5.7 Hz, 1H), 7.62 (d, J=8.4 Hz, 1H), 7.33 (s, 1H), 7.21 (s, 1H), 6.66 (d, J=5.7 Hz, 1H), 6.48-6.44 (m, 1H), 3.93 (s, 3H), 3.84 (s, 3H), 3.61-3.56 (m, 2H), 3.07-3.02 (m, 4H), 2.76-2.64 (m, 6H).

### Example S138. Compound 138

### Step 1: Synthesis of 5-(6-chloro-1-[[2-(trimethylsilyl)ethoxy]methyl]pyrrolo[2,3-b]pyridin-3-yl)-4,6-dimethoxypyrimidine

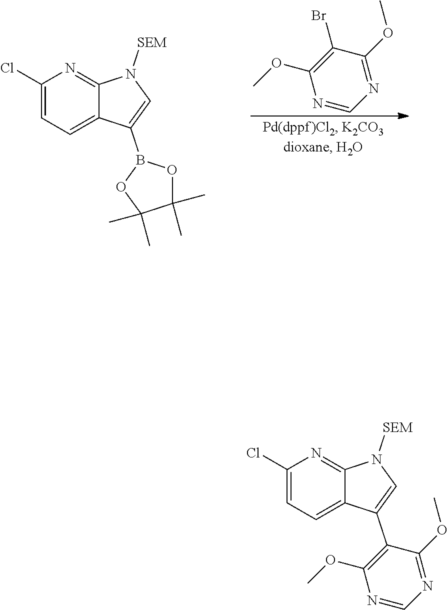

To a solution of 6-chloro-3-(4,4,5,5-tetramethyl-1,3,2-dioxaborolan-2-yl)-1-[[2-(trimethylsilyl)ethoxy]methyl]pyrrolo[2,3-b]pyridine (500.0 mg, 1.22 mmol) in 1,4-dioxane/H₂O (20.0 mL/5.0 mL) was added 5-bromo-4,6-dimethoxypyrimidine (267.9 mg, 1.22 mmol), K₂CO₃ (507.1 mg, 3.67 mmol) and Pd(dppf)Cl₂ (89.4 mg, 0.12 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash chromatography with petroleum ether/ethyl acetate (5/1, v/v) to afford 5-(6-chloro-1-[[2-(trimethylsilyl)ethoxy]methyl]pyrrolo[2,3-b]pyridin-3-yl)-4,6-dimethoxypyrimidine (240.0 mg, 46%) as a colorless oil. LCMS (ESI, m/z): [M+H]⁺=421.1.

### Step 2: Synthesis of 3-(4,6-dimethoxypyrimidin-5-yl)-1-[[2-(trimethylsilyl)ethoxy]methyl]pyrrolo[2,3-b]pyridin-6-amine

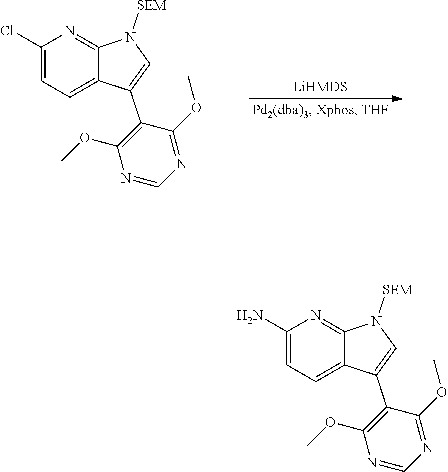

To a solution of 5-(6-chloro-1-[[2-(trimethylsilyl)ethoxy]methyl]pyrrolo[2,3-b]pyridin-3-yl)-4,6-dimethoxypyrimidine (110.0 mg, 0.26 mmol) in THF (8.0 mL) was added XPhos (24.9 mg, 0.05 mmol), Pd₂(dba)₃ (15.0 mg, 0.03 mmol) and LiHMDS (0.4 mL, 1 mol/L) at room temperature under N₂. The resulting mixture was stirred at 65° C. for 0.5 h. After the reaction was completed, the resulting mixture was concentrated under reduced pressure. The residue was purified by flash chromatography with petroleum ether/ethyl acetate (3/1, v/v) to afford 3-(4,6-dimethoxypyrimidin-5-yl)-1-[[2-(trimethylsilyl)ethoxy]methyl]pyrrolo[2,3-b]pyridin-6-amine (100.0 mg, 95%) as a brown yellow solid. LCMS (ESI, m/z): [M+H]⁺=402.2.

### Step 3: Synthesis of tert-butyl 4-[2-([[3-(4,6-dimethoxypyrimidin-5-yl)-1-[[2-(trimethylsilyl)ethoxy]methyl]pyrrolo[2,3-b]pyridin-6-yl]carbamoyl]amino)ethyl]piperazine-1-carboxylate

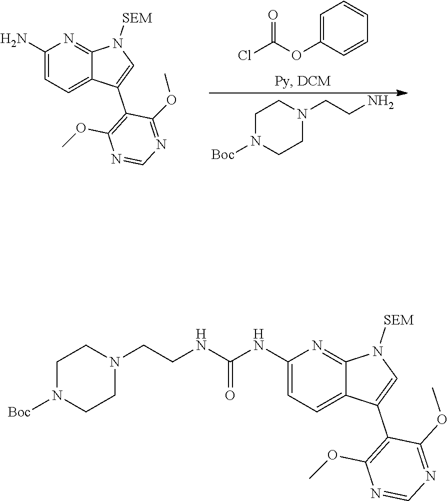 [SMILES: COc1ncnc(OC)c1-c1cn(COCC[Si](C)(C)C)c2nc(N)ccc12 ; CC(C)(C)OC(=O)N1CCN(CCN)CC1 ; O=C(Cl)Oc1ccccc1 ; COc1ncnc(OC)c1-c1cn(COCC[Si](C)(C)C)c2nc(NC(=O)NCCN3CCN(C(=O)OC(C)(C)C)CC3)ccc12]

To a solution of 3-(4,6-dimethoxypyrimidin-5-yl)-1-[[2-(trimethylsilyl)ethoxy]methyl]pyrrolo[2,3-b]pyridin-6-amine (180.0 mg, 0.45 mmol) in DCM (10.0 mL) was added pyridine (143.6 mg, 1.79 mmol) and phenyl chloroformate (84.2 mg, 0.54 mmol) at room temperature. The resulting mixture was stirred at room temperature for 3 h. The resulting mixture was concentrated under reduced pressure. To the above residue was added tert-butyl 4-(2-aminoethyl)piperazine-1-carboxylate (308.4 mg, 1.35 mmol) and pyridine (10.0 mL) at room temperature. The resulting mixture was stirred at 60° C. for another 2 h. After the reaction was completed, the resulting mixture was concentrated under reduced pressure. The residue was purified by flash column chromatography with DCM/MeOH (20/1, v/v) to afford tert-butyl 4-[2-([[3-(4,6-dimethoxypyrimidin-5-yl)-1-[[2-(trimethylsilyl)ethoxy]methyl]pyrrolo[2,3-b]pyridin-6-yl]carbamoyl]amino)ethyl]piperazine-1-carboxylate (200.0 mg, 67%) as a white solid. LCMS (ESI, m/z): [M+H]⁺=657.3.

### Step 4: Synthesis of 3-[3-(4,6-dimethoxypyrimidin-5-yl)-1H-pyrrolo[2,3-b]pyridin-6-yl]-1-[2-(piperazin-1-yl)ethyl]urea (Compound 138)

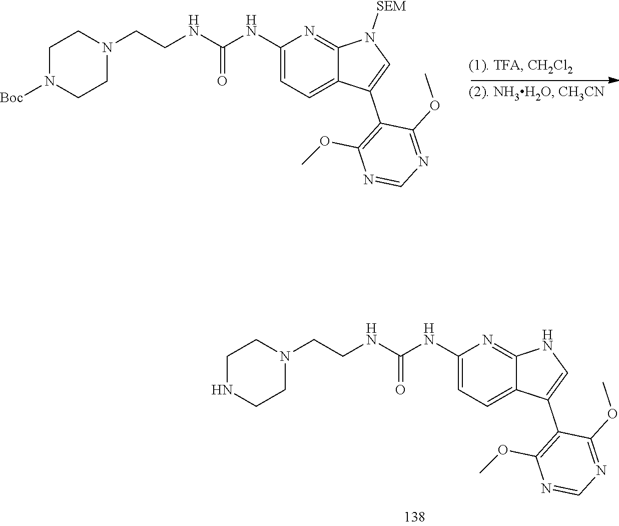 [SMILES: COc1ncnc(OC)c1-c1cn(COCC[Si](C)(C)C)c2nc(NC(=O)NCCN3CCN(C(=O)OC(C)(C)C)CC3)ccc12 ; COc1ncnc(OC)c1-c1c[nH]c2nc(NC(=O)NCCN3CCNCC3)ccc12]

To a solution of tert-butyl 4-[2-([[3-(4,6-dimethoxypyrimidin-5-yl)-1-[[2-(trimethylsilyl)ethoxy]methyl]pyrrolo[2,3-b]pyridin-6-yl]carbamoyl]amino)ethyl]piperazine-1-carboxylate (180.0 mg, 0.27 mmol) in DCM (3.0 mL) was added TFA (3.0 mL) at room temperature. The resulting mixture was stirred at room temperature for 3 h. The resulting mixture was concentrated under reduced pressure. To the residue in CH₃CN (3.0 mL) was added NH₃·H₂O (3.0 mL) at room temperature. The resulting mixture was stirred at room temperature for another 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 24% B to 34% B in 7 min, 254/220 nm) to afford 3-[3-(4,6-dimethoxypyrimidin-5-yl)-1H-pyrrolo[2,3-b]pyridin-6-yl]-1-[2-(piperazin-1-yl)ethyl]urea (33.5 mg, 28%) as a white solid. LCMS (ESI, m/z): [M+H]⁺=427.3. ¹H NMR (400 MHz, DMSO-d₆): δ 11.53 (s, 1H), 9.20 (s, 1H), 8.46 (s, 1H), 8.32 (s, 1H), 7.62 (d, J=8.8 Hz, 1H), 7.37 (s, 1H), 7.06 (d, J=8.8 Hz, 1H), 3.91 (s, 6H), 3.39-3.30 (m, 3H), 2.71 (s, 4H), 2.51-2.50 (m, 2H), 2.45-2.37 (m, 4H).

### Example S139. Compound 139

### Step 1: Synthesis of tert-butyl N-[3-(5-methoxy-1,3-benzothiazol-6-yl)-1-[[2-(trimethylsilyl)ethoxy]methyl]pyrrolo[2,3-b]pyridin-6-yl]carbamate

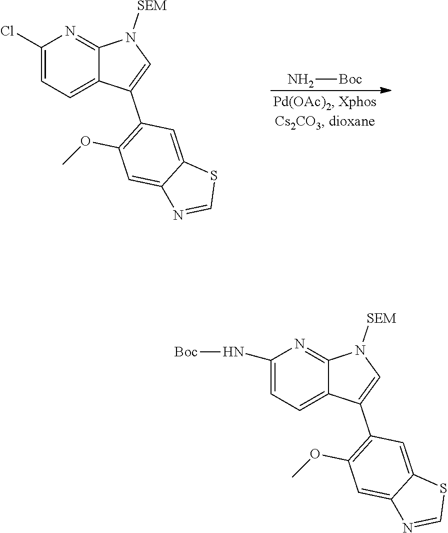 [SMILES: COc1cc2ncsc2cc1-c1cn(COCC[Si](C)(C)C)c2nc(Cl)ccc12 ; CC(C)(C)OC(N)=O ; COc1cc2ncsc2cc1-c1cn(COCC[Si](C)(C)C)c2nc(NC(=O)OC(C)(C)C)ccc12]

To a solution of 6-(6-chloro-1-[[2-(trimethylsilyl)ethoxy]methyl]pyrrolo[2,3-b]pyridin-3-yl)-5-methoxy-1,3-benzothiazole (380.0 mg, 0.85 mmol) in 1.4-dioxane (10.0 mL) was added tert-butyl carbamate (139.7 mg, 1.19 mmol), Xphos (81.2 mg, 0.17 mmol), Cs₂CO₃ (694.0 mg, 2.13 mmol) and Pd(OAc)₂ (19.1 mg, 0.09 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford tert-butyl N-[3-(5-methoxy-1,3-benzothiazol-6-yl)-1-[[2-(trimethylsilyl)ethoxy]methyl]pyrrolo[2,3-b]pyridin-6-yl]carbamate (420.0 mg, 93%) as a yellow oil. LCMS (ESI, m/z): [M+H]⁺=527.2.

### Step 2: Synthesis of 3-(5-methoxy-1,3-benzothiazol-6-yl)-1-[[2-(trimethylsilyl)ethoxy]methyl]pyrrolo[2,3-b]pyridin-6-amine

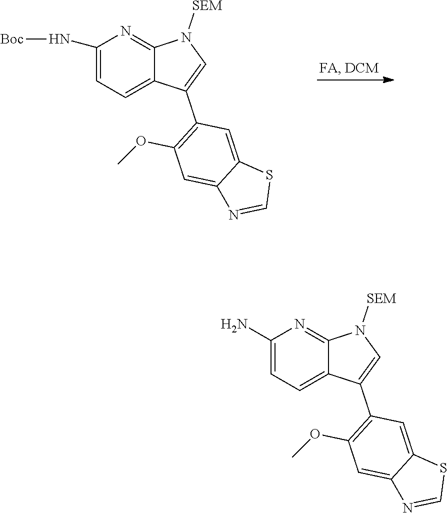 [SMILES: COc1cc2ncsc2cc1-c1cn(COCC[Si](C)(C)C)c2nc(NC(=O)OC(C)(C)C)ccc12 ; COc1cc2ncsc2cc1-c1cn(COCC[Si](C)(C)C)c2nc(N)ccc12]

To a solution of tert-butyl N-[3-(5-methoxy-1,3-benzothiazol-6-yl)-1-[[2-(trimethylsilyl)ethoxy]methyl]pyrrolo[2,3-b]pyridin-6-yl]carbamate (470.0 mg, 0.89 mmol) in CH₂Cl₂ (10.0 mL) was added formic acid (FA, 10.0 mL) at room temperature. The resulting mixture was stirred at room temperature for 2 h. After the reaction was completed, the pH value of the mixture was adjusted to 8 with NaHCO₃ solution. The resulting mixture was diluted with H₂O and extracted with ethyl acetate. The combined organic layer was washed with brine, dried over anhydrous sodium sulfate and filtered. The filtrate was concentrated under reduced pressure to afford 3-(5-methoxy-1,3-benzothiazol-6-yl)-1-[[2-(trimethylsilyl)ethoxy]methyl]pyrrolo[2,3-b]pyridin-6-amine (380.0 mg, crude) as a brown oil. LCMS (ESI, m/z): [M+H]⁺=427.2.

### Step 3: Synthesis of tert-butyl 4-[2-([[3-(5-methoxy-1,3-benzothiazol-6-yl)-1-[[2-(trimethylsilyl)ethoxy]methyl]pyrrolo[2,3-b]pyridin-6-yl]carbamoyl]amino)ethyl]piperazine-1-carboxylate

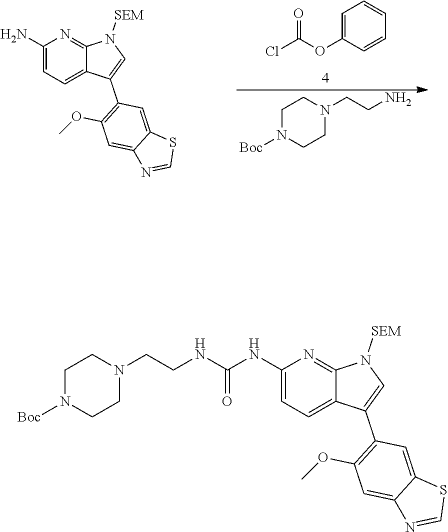 [SMILES: COc1cc2ncsc2cc1-c1cn(COCC[Si](C)(C)C)c2nc(N)ccc12 ; CC(C)(C)OC(=O)N1CCN(CCN)CC1 ; O=C(Cl)Oc1ccccc1 ; COc1cc2ncsc2cc1-c1cn(COCC[Si](C)(C)C)c2nc(NC(=O)NCCN3CCN(C(=O)OC(C)(C)C)CC3)ccc12]

To a solution of 3-(5-methoxy-1,3-benzothiazol-6-yl)-1-[[2-(trimethylsilyl)ethoxy]methyl]pyrrolo[2,3-b]pyridin-6-amine (290.0 mg, crude) in DCM (8.0 mL) was added pyridine (215.1 mg, 2.72 mmol) and phenyl chloroformate (255.4 mg, 1.63 mmol) at room temperature. The resulting mixture was stirred at room temperature for 16 h. The resulting mixture was concentrated under reduced pressure. To the above mixture was added tert-butyl 4-(2-aminoethyl)piperazine-1-carboxylate (935.3 mg, 4.08 mmol) and pyridine (8.0 mL) at room temperature. The resulting mixture was stirred at 60° C. for another 2 h. After the reaction was completed, the resulting mixture was concentrated under reduced pressure. The residue was purified by flash column chromatography with CH₂Cl₂/CH₃OH (15/1, v/v) to afford tert-butyl 4-[2-([[3-(5-methoxy-1,3-benzothiazol-6-yl)-1-[[2-(trimethylsilyl)ethoxy]methyl]pyrrolo[2,3-b]pyridin-6-yl]carbamoyl]amino)ethyl]piperazine-1-carboxylate (155.0 mg, 33%) as a yellow oil. LCMS (ESI, m/z): [M+H]⁺=682.3.

### Step 4: Synthesis of 3-[3-(5-methoxy-1,3-benzothiazol-6-yl)-1H-pyrrolo[2,3-b]pyridin-6-yl]-1-[2-(piperazin-1-yl)ethyl]urea; bis(formic acid) (Compound 139)

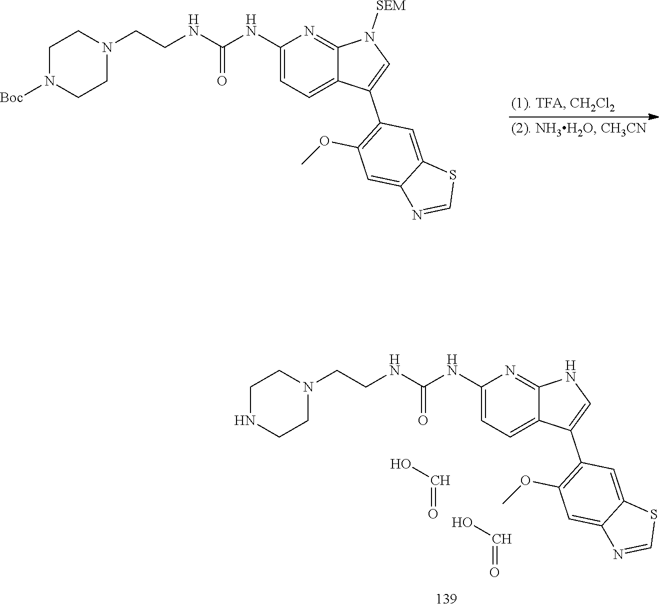

To a solution of tert-butyl 4-[2-([[3-(5-methoxy-1,3-benzothiazol-6-yl)-1-[[2-(trimethylsilyl)ethoxy]methyl]pyrrolo[2,3-b]pyridin-6-yl]carbamoyl]amino)ethyl]piperazine-1-carboxylate (155.0 mg, 0.23 mmol) in CH₂Cl₂ (8.0 mL) was added TFA (8.0 mL) at room temperature. The resulting mixture was stirred at room temperature for 3 h. The resulting mixture was concentrated under reduced pressure. To the residue in CH₃CN (8.0 mL) was added NH₃·H₂O (8.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 7% B to 20% B in 8 min; 254/220 nm) to afford 3-[3-(5-methoxy-1,3-benzothiazol-6-yl)-1H-pyrrolo[2,3-b]pyridin-6-yl]-1-[2-(piperazin-1-yl)ethyl]urea; bis(formic acid) (12.9 mg, 9%) as a white semi-solid. LCMS (ESI, m/z): [M+H]⁺=452.3. ¹H NMR (400 MHz, DMSO-d₆): δ 11.58 (s, 1H), 9.31 (s, 1H), 9.23 (s, 1H), 8.41 (s, 1H), 8.30 (d, J=3.6 Hz, 2H), 8.24 (s, 1H), 8.00 (d, J=8.4 Hz, 1H), 7.75 (s, 1H), 7.57 (d, J=2.4 Hz, 1H), 7.11 (d, J=8.8 Hz, 1H), 3.92 (s, 3H), 3.37-3.32 (m, 2H), 3.02-2.91 (m, 4H), 2.67-2.55 (m, 5H).

### Example S140. Compound 140

### Step 1: Synthesis of 5-(6-chloro-1-[[2-(trimethylsilyl)ethoxy]methyl]pyrrolo[2,3-b]pyridin-3-yl)-6-methoxy-1,3-benzothiazole

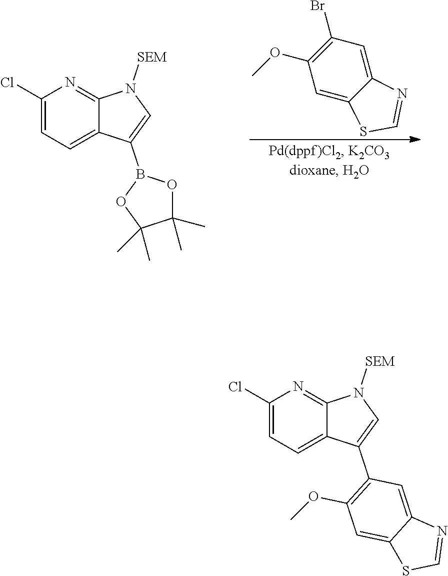 [SMILES: CC1(C)OB(c2cn(COCC[Si](C)(C)C)c3nc(Cl)ccc23)OC1(C)C ; COc1cc2scnc2cc1Br ; COc1cc2scnc2cc1-c1cn(COCC[Si](C)(C)C)c2nc(Cl)ccc12]

To a solution of 6-chloro-3-(4,4,5,5-tetramethyl-1,3,2-dioxaborolan-2-yl)-1-[[2-(trimethylsilyl)ethoxy]methyl]pyrrolo[2,3-b]pyridine (500.0 mg, 1.22 mmol) in dioxane/H₂O (20.0 mL/4.0 mL) was added 5-bromo-6-methoxy-1,3-benzothiazole (298.6 mg, 1.22 mmol), K₂CO₃ (507.1 mg, 3.67 mmol) and Pd(dppf)Cl₂ (89.5 mg, 0.12 mmol) at room temperature under N₂. The resulting mixture was stirred at 80° C. for 16 h under N₂. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 5-(6-chloro-1-[[2-(trimethylsilyl)ethoxy]methyl]pyrrolo[2,3-b]pyridin-3-yl)-6-methoxy-1,3-benzothiazole (250.0 mg, 48%) as a yellow oil. LCMS (ESI, m/z): [M+H]⁺=446.1.

### Step 2: Synthesis of tert-butyl N-[3-(6-methoxy-1,3-benzothiazol-5-yl)-1-[[2-(trimethylsilyl)ethoxy]methyl]pyrrolo[2,3-b]pyridin-6-yl]carbamate

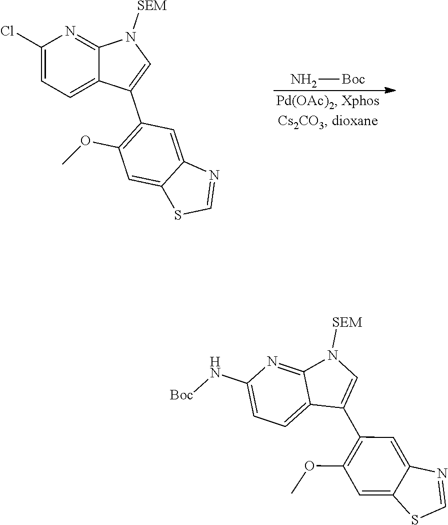 [SMILES: COc1cc2scnc2cc1-c1cn(COCC[Si](C)(C)C)c2nc(Cl)ccc12 ; CC(C)(C)OC(N)=O ; COc1cc2scnc2cc1-c1cn(COCC[Si](C)(C)C)c2nc(NC(=O)OC(C)(C)C)ccc12]

To a solution of 5-(6-chloro-1-[[2-(trimethylsilyl)ethoxy]methyl]pyrrolo[2,3-b]pyridin-3-yl)-6-methoxy-1,3-benzothiazole (230.0 mg, 0.52 mmol) in 1,4-dioxane (24.0 mL) was added tert-butyl carbamate (84.6 mg, 0.72 mmol), XPhos (49.2 mg, 0.11 mmol), Cs₂CO₃ (420.0 mg, 1.29 mmol) and Pd(OAc)₂ (11.6 mg, 0.05 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (1/3, v/v) to afford tert-butyl N-[3-(6-methoxy-1,3-benzothiazol-5-yl)-1-[[2-(trimethylsilyl)ethoxy]methyl]pyrrolo[2,3-b]pyridin-6-yl]carbamate (160.0 mg, 59%) as a yellow oil. LCMS (ESI, m/z): [M+H]⁺=527.2.

### Step 3: Synthesis of 3-(6-methoxy-1,3-benzothiazol-5-yl)-1-[[2-(trimethylsilyl)ethoxy]methyl]pyrrolo[2,3-b]pyridin-6-amine

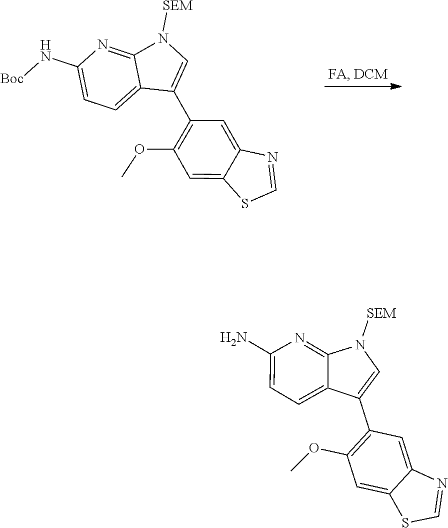 [SMILES: COc1cc2scnc2cc1-c1cn(COCC[Si](C)(C)C)c2nc(NC(=O)OC(C)(C)C)ccc12 ; COc1cc2scnc2cc1-c1cn(COCC[Si](C)(C)C)c2nc(N)ccc12]

To a solution of tert-butyl N-[3-(6-methoxy-1,3-benzothiazol-5-yl)-1-[[2-(trimethylsilyl)ethoxy]methyl]pyrrolo[2,3-b]pyridin-6-yl]carbamate (140.0 mg, 0.27 mmol) in DCM (15.0 mL) was added HCOOH (3.0 mL) at room temperature. The resulting mixture was stirred at room temperature for 30 min. After the reaction was completed, the reaction mixture was diluted with H₂O. The pH value of the mixture was adjusted to 8 with saturated NaHCO₃ solution. The mixture was extracted with CH₂Cl₂. The combined organic layer was washed with brine, dried over anhydrous sodium sulfate and filtered. The filtrate was evaporated in vacuo to afford 3-(6-methoxy-1,3-benzothiazol-5-yl)-1-[[2-(trimethylsilyl)ethoxy]methyl]pyrrolo[2,3-b]pyridin-6-amine (110.0 mg, crude) as a yellow oil. LCMS (ESI, m/z): [M+H]⁺=427.2.

### Step 4: Synthesis of tert-butyl 4-[2-([[3-(6-methoxy-1,3-benzothiazol-5-yl)-1-[[2-(trimethylsilyl)ethoxy]methyl]pyrrolo[2,3-b]pyridin-6-yl]carbamoyl]amino)ethyl]piperazine-1-carboxylate

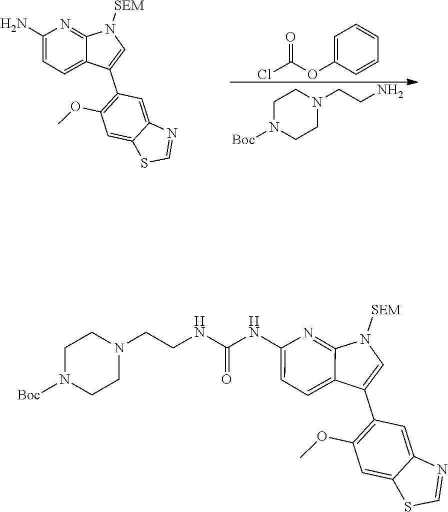

To a solution of 3-(6-methoxy-1,3-benzothiazol-5-yl)-1-[[2-(trimethylsilyl)ethoxy]methyl]pyrrolo[2,3-b]pyridin-6-amine (80.0 mg, crude) in DCM (8.0 mL) was added pyridine (60.1 mg, 0.75 mmol) and phenyl chloroformate (35.2 mg, 0.23 mmol) at room temperature. The resulting mixture was stirred at room temperature for 16 h under N₂. The resulting mixture was concentrated under reduced pressure. To the residue in pyridine (8.0 mL) was added tert-butyl 4-(2-aminoethyl)piperazine-1-carboxylate (215.0 mg, 0.94 mmol) at room temperature. The resulting mixture was stirred at 60° C. for another 4 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford tert-butyl 4-[2-([[3-(6-methoxy-1,3-benzothiazol-5-yl)-1-[[2-(trimethylsilyl)ethoxy]methyl]pyrrolo[2,3-b]pyridin-6-yl]carbamoyl]amino)ethyl]piperazine-1-carboxylate (120.0 mg, 94%) as a brown solid. LCMS (ESI, m/z): [M+H]⁺=682.3.

### Step 5: Synthesis of 3-[3-(6-methoxy-1,3-benzothiazol-5-yl)-1H-pyrrolo[2,3-b]pyridin-6-yl]-1-[2-(piperazin-1-yl)ethyl]urea (Compound 140)

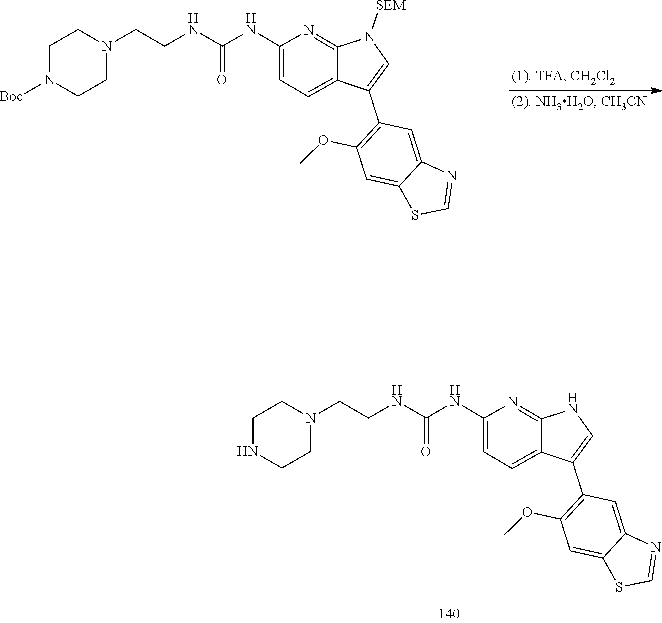 [SMILES: COc1cc2scnc2cc1-c1cn(COCC[Si](C)(C)C)c2nc(NC(=O)NCCN3CCN(C(=O)OC(C)(C)C)CC3)ccc12 ; COc1cc2scnc2cc1-c1c[nH]c2nc(NC(=O)NCCN3CCNCC3)ccc12]

To a solution of tert-butyl 4-[2-([[3-(6-methoxy-1,3-benzothiazol-5-yl)-1-[[2-(trimethylsilyl)ethoxy]methyl]pyrrolo[2,3-b]pyridin-6-yl]carbamoyl]amino)ethyl]piperazine-1-carboxylate (120.0 mg, 0.18 mmol) in CH₂Cl₂ (10.0 mL) was added TFA (10.0 mL) at room temperature. The resulting mixture was stirred at room temperature for 2 h. The resulting mixture was concentrated under reduced pressure. To the residue in CH₃CN (10.0 mL) was added NH₃·H₂O (10.0 mL) at room temperature. The resulting mixture was stirred at room temperature for another 4 h. After the reaction was completed, the resulting mixture was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 12% B to 42% B in 9 min; 254 nm) to afford 3-[3-(6-methoxy-1,3-benzothiazol-5-yl)-1H-pyrrolo[2,3-b]pyridin-6-yl]-1-[2-(piperazin-1-yl)ethyl]urea (22.4 mg, 28%) as a yellow solid. LCMS (ESI, m/z): [M+H]⁺=452.3. ¹H NMR (400 MHz, CD₃OD): δ 9.09 (d, J=1.6 Hz, 1H), 8.16 (d, J=1.6 Hz, 1H), 8.02-8.00 (m, 1H), 7.74 (d, J=3.6 Hz, 1H), 7.55 (d, J=0.8 Hz, 1H), 6.79 (d, J=8.4 Hz, 1H), 3.96 (s, 3H), 3.57-3.49 (m, 2H), 2.94-2.92 (m, 4H), 2.79-2.60 (m, 6H).

### Example S141. Compound 141

### Step 1: Synthesis of 5-bromo-6-methoxy-1-((2-(trimethylsilyl)ethoxy)methyl)-1H-pyrrolo[2,3-b]pyridine

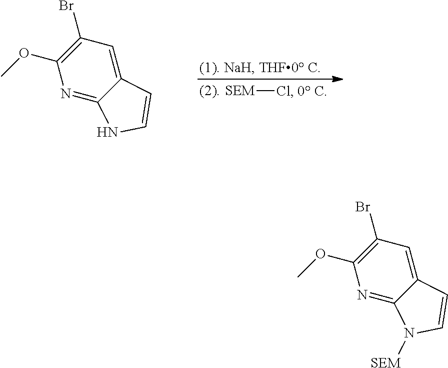

To a solution of 5-bromo-6-methoxy-1H-pyrrolo[2,3-b]pyridine (500.0 mg, 2.20 mmol) in THE (5.0 mL) was added NaH (158.5 mg, 60%) at 0° C. under N₂. The resulting mixture was stirred at 0° C. for 1 h under N₂. Then SEM-Cl (440.5 mg, 2.64 mmol) was added to the mixture at 0° C. under N₂. The resulting mixture was stirred at 0° C. for another 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (93/7, v/v) to afford 5-bromo-6-methoxy-1-((2-(trimethylsilyl)ethoxy)methyl)-1H-pyrrolo[2,3-b]pyridine (620.0 mg, 80%) as a light yellow oil. LCMS (ESI, m/z): [M+H]⁺=357.1.

### Step 2: Synthesis of 6-chloro-6′-methoxy-1,1′-bis((2-(trimethylsilyl)ethoxy)methyl)-1H,1′H-3,5′-bipyrrolo[2,3-b]pyridine

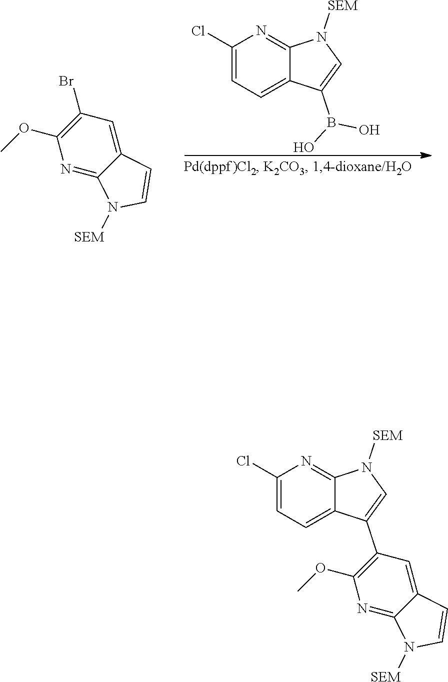 [SMILES: COc1nc2c(ccn2COCC[Si](C)(C)C)cc1Br ; C[Si](C)(C)CCOCn1cc(B(O)O)c2ccc(Cl)nc21 ; COc1nc2c(ccn2COCC[Si](C)(C)C)cc1-c1cn(COCC[Si](C)(C)C)c2nc(Cl)ccc12]

To a solution of 5-bromo-6-methoxy-1-((2-(trimethylsilyl)ethoxy)methyl)-1H-pyrrolo[2,3-b]pyridine (320.0 mg, 0.90 mmol) in 1,4-dioxane/H₂O (10.0/2.0 mL) was added (6-chloro-1-((2-(trimethylsilyl)ethoxy)methyl)-1H-pyrrolo[2,3-b]pyridin-3-yl)boronic acid (366.1 mg, 0.90 mmol), K₂CO₃ (371.3 mg, 2.69 mmol) and Pd(dppf)Cl₂ (65.5 mg, 0.09 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (93/7, v/v) to afford 6-chloro-6′-methoxy-1,1′-bis ((2-(trimethylsilyl)ethoxy)methyl)-1H,1′H-3,5′-bipyrrolo[2,3-b]pyridine (210.0 mg, 41%) as a light yellow oil. LCMS (ESI, m/z): [M+H]⁺=559.2.

### Step 3: Synthesis of the mixture of (1S,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide and (1R,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide

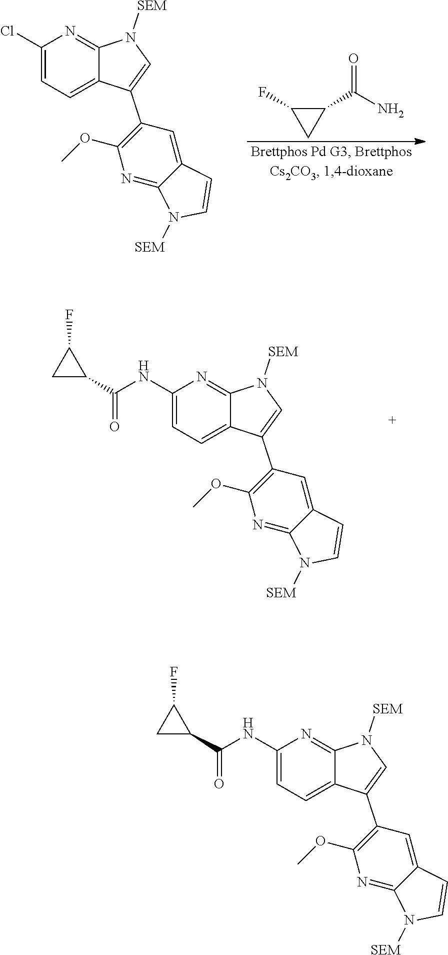 [SMILES: COc1nc2c(ccn2COCC[Si](C)(C)C)cc1-c1cn(COCC[Si](C)(C)C)c2nc(Cl)ccc12 ; NC(=O)[C@@H]1C[C@@H]1F ; COc1nc2c(ccn2COCC[Si](C)(C)C)cc1-c1cn(COCC[Si](C)(C)C)c2nc(NC(=O)[C@@H]3C[C@@H]3F)ccc12 ; COc1nc2c(ccn2COCC[Si](C)(C)C)cc1-c1cn(COCC[Si](C)(C)C)c2nc(NC(=O)[C@H]3C[C@@H]3F)ccc12]

To a solution of 6-chloro-6′-methoxy-1,1′-bis ((2-(trimethylsilyl)ethoxy)methyl)-1H,1′H-3,5′-bipyrrolo[2,3-b]pyridine (210.0 mg, 0.38 mmol) in 1,4-dioxane (10.0 mL) was added (1S,2S)-2-fluorocyclopropane-1-carboxamide (193.6 mg, 1.87 mmol), Cs₂CO₃ (367.0 mg, 1.13 mmol), BrettPhos (40.3 mg, 0.08 mmol) and BrettPhos Pd G3 (34.0 mg, 0.04 mmol) at room temperature under N₂, The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (83/17, v/v) to afford the mixture of (1S,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide and (1R,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide (110.0 mg, 49%) as a yellow oil. LCMS (ESI, m/z): [M+H]⁺=626.3.

### Step 4: Synthesis of (1R,2S)-2-fluoro-N-(6′-methoxy-1H,1′H-[3,5′-bipyrrolo[2,3-b]pyridin]-6-yl)cyclopropane-1-carboxamide (Compound 141)

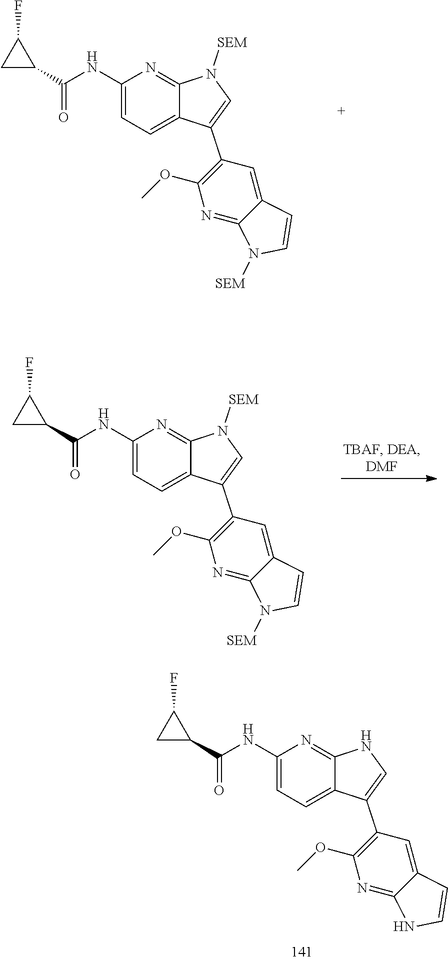 [SMILES: COc1nc2c(ccn2COCC[Si](C)(C)C)cc1-c1cn(COCC[Si](C)(C)C)c2nc(NC(=O)[C@@H]3C[C@@H]3F)ccc12 ; COc1nc2c(ccn2COCC[Si](C)(C)C)cc1-c1cn(COCC[Si](C)(C)C)c2nc(NC(=O)[C@H]3C[C@@H]3F)ccc12 ; COc1nc2[nH]ccc2cc1-c1c[nH]c2nc(NC(=O)[C@H]3C[C@@H]3F)ccc12]

To a solution of the mixture of (1S,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide and (1R,2S)-2-fluoro-N-(6′-methoxy-1,1′-bis((2-(trimethylsilyl)ethoxy)methyl)-1H,1′H-[3,5′-bipyrrolo[2,3-b]pyridin]-6-yl)cyclopropane-1-carboxamide (50.0 mg, 0.08 mmol) in DMF (2.0 mL) was added TBAF (62.7 mg, 0.24 mmol) and DEA (24.1 mg, 0.40 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) and then Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 30% B to 40% B in 11 min; 254 nm) to afford (1R,2S)-2-fluoro-N-(6′-methoxy-1H,1′H-[3,5′-bipyrrolo[2,3-b]pyridin]-6-yl)cyclopropane-1-carboxamide (3.1 mg, 10%) as a white solid. LCMS (ESI, m/z): [M+H]⁺=366.1. ¹H NMR (300 MHz, DMSO-d₆): δ 11.56-11.46 (m, 2H), 10.76 (s, 1H), 8.05-8.00 (m, 2H), 7.85 (d, J=7.8 Hz, 1H), 7.58 (s, 1H), 7.22-7.20 (m, 1H), 6.40 (s, 1H), 5.01-4.79 (m, 1H), 3.94 (s, 3H), 2.60-2.51 (m, 1H), 1.57-1.45 (m, 1H), 1.31-1.20 (m, 1H).

### Example S142. Compound 142

### Step 1: Synthesis of 6-chloro-3-(4,6-dimethoxy-2-methylpyrimidin-5-yl)-1-((2-(trimethylsilyl)ethoxy)methyl)-1H-pyrrolo[2,3-b]pyridine

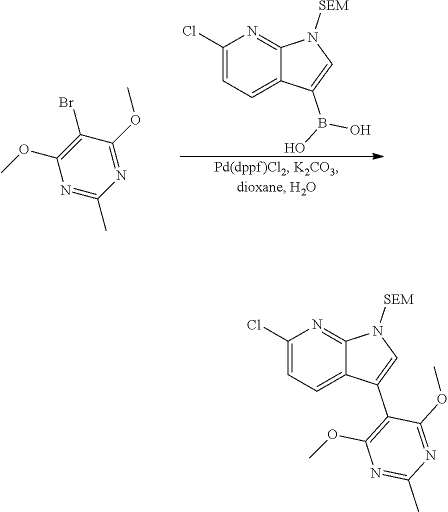 [SMILES: COc1nc(C)nc(OC)c1Br ; C[Si](C)(C)CCOCn1cc(B(O)O)c2ccc(Cl)nc21 ; COc1nc(C)nc(OC)c1-c1cn(COCC[Si](C)(C)C)c2nc(Cl)ccc12]

To a solution of 5-bromo-4,6-dimethoxy-2-methylpyrimidine (400.0 mg, 1.72 mmol) in dioxane/H₂O (16.0/4.0 mL) was added (6-chloro-1-((2-(trimethylsilyl)ethoxy)methyl)-1H-pyrrolo[2,3-b]pyridin-3-yl)boronic acid (560.6 mg, 1.72 mmol), K₂CO₃ (711.6 mg, 5.15 mmol) and Pd(dppf)Cl₂ (125.6 mg, 0.17 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 6-chloro-3-(4,6-dimethoxy-2-methylpyrimidin-5-yl)-1-((2-(trimethylsilyl)ethoxy)methyl)-1H-pyrrolo[2,3-b]pyridine (270.0 mg, 20%) as a yellow oil. LCMS (ESI, m/z): [M+H]⁺=435.2.

### Step 2: Synthesis of (1S,2S)—N-(3-(4,6-dimethoxy-2-methylpyrimidin-5-yl)-1-((2-(trimethylsilyl)ethoxy)methyl)-1H-pyrrolo[2,3-b]pyridin-6-yl)-2-fluorocyclopropane-1-carboxamide

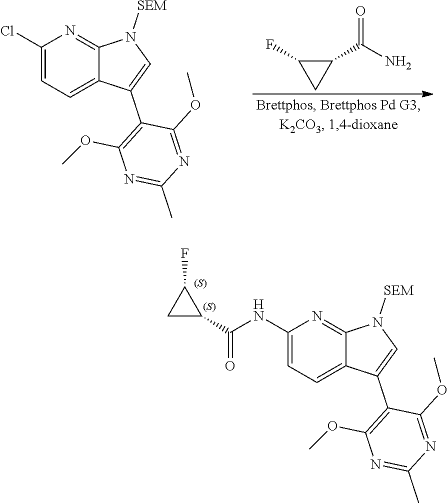

To a solution of 6-chloro-3-(4,6-dimethoxy-2-methylpyrimidin-5-yl)-1-((2-(trimethylsilyl)ethoxy)methyl)-1H-pyrrolo[2,3-b]pyridine (100.0 mg, 0.23 mmol) in 1,4-dioxane (5.0 mL) was added (1S,2S)-2-fluorocyclopropane-1-carboxamide (118.5 mg, 1.15 mmol), K₂CO₃ (95.3 mg, 0.69 mmol), Brettphos (24.7 mg, 0.05 mmol) and BrettPhos Pd G3 (20.8 mg, 0.02 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-(3-(4,6-dimethoxy-2-methylpyrimidin-5-yl)-1-((2-(trimethylsilyl)ethoxy)methyl)-1H-pyrrolo[2,3-b]pyridin-6-yl)-2-fluorocyclopropane-1-carboxamide (60.0 mg, 52%) as a yellow solid. LCMS (ESI, m/z): [M+H]⁺=502.2.

### Step 3: Synthesis of (1S,2S)—N-(3-(4,6-dimethoxy-2-methylpyrimidin-5-yl)-1H-pyrrolo[2,3-b]pyridin-6-yl)-2-fluorocyclopropane-1-carboxamide (Compound 142)

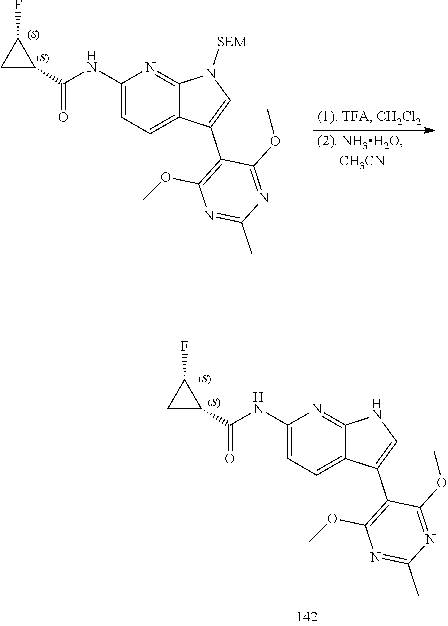

To a solution of (1S,2S)—N-(3-(4,6-dimethoxy-2-methylpyrimidin-5-yl)-1-((2-(trimethylsilyl)ethoxy)methyl)-1H-pyrrolo[2,3-b]pyridin-6-yl)-2-fluorocyclopropane-1-carboxamide (50.0 mg, 0.10 mmol) in CH₂Cl₂ (2.0 mL) was added TFA (2.0 mL) at room temperature. The resulting mixture was stirred at room temperature for 3 h. The resulting mixture was concentrated under reduced pressure. To the residue in CH₃CN (2.0 mL) was added NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 36% to 46% in 8 min; 254 nm) to afford (1S,2S)—N-(3-(4,6-dimethoxy-2-methylpyrimidin-5-yl)-1H-pyrrolo[2,3-b]pyridin-6-yl)-2-fluorocyclopropane-1-carboxamide (13.6 mg, 36%) as a white solid. LCMS (ESI, m/z): [M+H]⁺=372.2. ¹H NMR (300 MHz, DMSO-d₆): δ 11.59 (s, 1H), 10.59 (s, 1H), 7.84 (d, J=8.4 Hz, 1H), 7.68 (d, J=8.7 Hz, 1H), 7.42 (d, J=2.7 Hz, 1H), 5.05-4.78 (m, 1H), 3.88 (s, 6H), 2.52 (s, 3H), 2.27-2.20 (m, 1H), 1.72-1.60 (m, 1H), 1.25-1.08 (m, 1H).

### Example S143. Compound 143

### Step 1: Synthesis of tert-butyl N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)carbamate

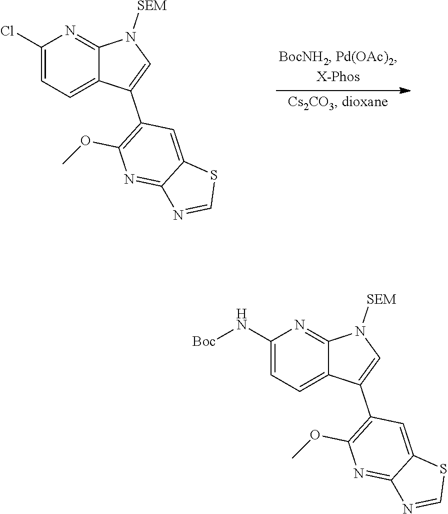 [SMILES: COc1nc2ncsc2cc1-c1cn(COCC[Si](C)(C)C)c2nc(Cl)ccc12 ; COc1nc2ncsc2cc1-c1cn(COCC[Si](C)(C)C)c2nc(NC(=O)OC(C)(C)C)ccc12]

To a mixture of 6-chloro-3-[5-methoxy-[1,3]thiazolo[4,5-b]pyridin-6-yl]-1-[[2-(trimethylsilyl)ethoxy]methyl]pyrrolo[2,3-b]pyridine (300.0 mg, 0.67 mmol), BocNH₂ (235.8 mg, 2.01 mmol) and Cs₂CO₃ (655.9 mg, 2.01 mmol) in dioxane (5.0 mL) were added Pd(OAc)₂ (15.1 mg, 0.07 mmol) and X-Phos (63.9 mg, 0.14 mmol). The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)carbamate (330.0 mg, 93%) as a yellow solid. LCMS (ESI, m/z): [M+H]⁺=528.2.

### Step 2: Synthesis of 3-(5-methoxythiazolo[4,5-b]pyridin-6-yl)-1-((2-(trimethylsilyl)ethoxy)methyl)-1H-pyrrolo[2,3-b]pyridin-6-amine

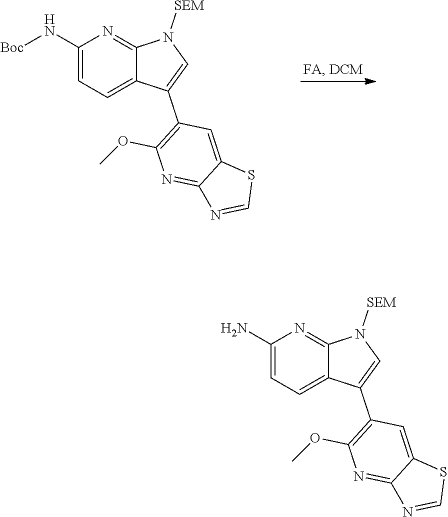 [SMILES: COc1nc2ncsc2cc1-c1cn(COCC[Si](C)(C)C)c2nc(NC(=O)OC(C)(C)C)ccc12 ; COc1nc2ncsc2cc1-c1cn(COCC[Si](C)(C)C)c2nc(N)ccc12]

A mixture of tert-butyl N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)carbamate (300.0 mg, 0.57 mmol) and formic acid (10.0 mL) in DCM (20.0 mL) was stirred at room temperature for 16 h. After the reaction was completed, the pH value of the mixture was adjusted to 7 with NaHCO₃ solution. The resulting mixture was diluted with H₂O and extracted with ethyl acetate. The combined organic layer was washed with brine, dried over anhydrous sodium sulfate and filtered. The filtrate was concentrated under reduced pressure to afford 3-(5-methoxythiazolo[4,5-b]pyridin-6-yl)-1-((2-(trimethylsilyl)ethoxy)methyl)-1H-pyrrolo[2,3-b]pyridin-6-amine (200.0 mg, crude) as a yellow oil. LCMS (ESI, m/z): [M+H]⁺=428.1.

### Step 3: Synthesis of 1-(2-(4-ethylpiperazin-1-yl)ethyl)-3-(3-(5-methoxythiazolo[4,5-b]pyridin-6-yl)-1-((2-(trimethylsilyl)ethoxy)methyl)-1H-pyrrolo[2,3-b]pyridin-6-yl)urea

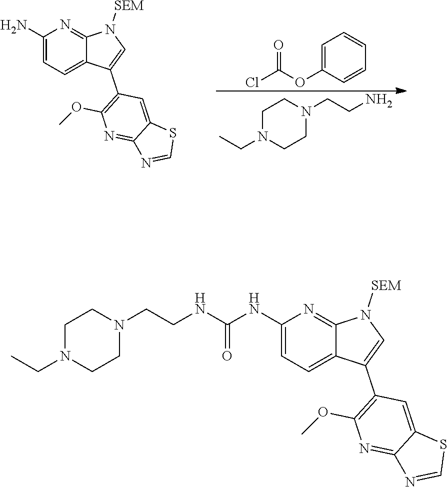

To a mixture of 3-[5-methoxy-[1,3]thiazolo[4,5-b]pyridin-6-yl]-1-[[2-(trimethylsilyl)ethoxy]methyl]pyrrolo[2,3-b]pyridin-6-amine (100.0 mg, 0.23 mmol) and pyridine (74.0 mg, 0.94 mmol) in DCM (2.0 mL) was added phenyl chloroformate (73.2 mg, 0.46 mmol). The resulting mixture was stirred at room temperature for 3 h. The mixture was evaporated in vacuo. To the above residue was added pyridine (4.0 mL) and 2-(4-ethylpiperazin-1-yl)ethanamine (183.9 mg, 1.17 mmol). The resulting mixture was stirred at 60° C. for another 16 h. After the reaction was completed, the mixture was concentrated under reduced pressure. The residue was purified by flash column chromatography with petroleum ether/ethyl acetate (1/5, v/v) to afford 1-(2-(4-ethylpiperazin-1-yl)ethyl)-3-(3-(5-methoxythiazolo[4,5-b]pyridin-6-yl)-1-((2-(trimethylsilyl)ethoxy)methyl)-1H-pyrrolo[2,3-b]pyridin-6-yl)urea (35.0 mg, 25%) as a yellow solid. LCMS (ESI, m/z): [M+H]⁺=611.3.

### Step 4: Synthesis of 1-(2-(4-ethylpiperazin-1-yl)ethyl)-3-(3-(5-methoxythiazolo[4,5-b]pyridin-6-yl)-1H-pyrrolo[2,3-b]pyridin-6-yl)urea (Compound 143)

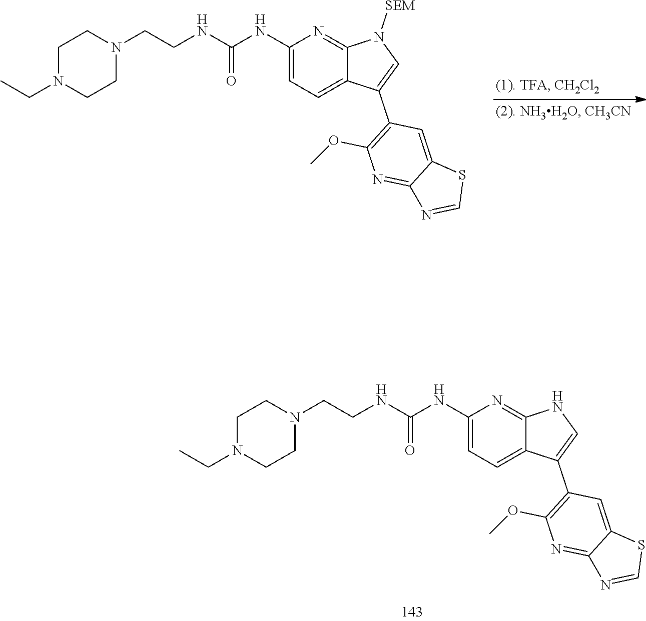 [SMILES: CCN1CCN(CCNC(=O)Nc2ccc3c(-c4cc5scnc5nc4OC)cn(COCC[Si](C)(C)C)c3n2)CC1 ; CCN1CCN(CCNC(=O)Nc2ccc3c(-c4cc5scnc5nc4OC)c[nH]c3n2)CC1]

To a solution of 1-(2-(4-ethylpiperazin-1-yl)ethyl)-3-(3-(5-methoxythiazolo[4,5-b]pyridin-6-yl)-1-((2-(trimethylsilyl)ethoxy)methyl)-1H-pyrrolo[2,3-b]pyridin-6-yl)urea (35.0 mg, 0.06 mmol) in CH₂Cl₂ (2.0 mL) was added TFA (2.0 mL) at room temperature. The resulting mixture was stirred at room temperature for 4 h. The resulting mixture was concentrated under reduced pressure. To the residue was added CH₃CN (2.0 mL) and NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 26% B to 35% B in 7 min; 254 nm) to afford 1-(2-(4-ethylpiperazin-1-yl)ethyl)-3-(3-(5-methoxythiazolo[4,5-b]pyridin-6-yl)-1H-pyrrolo[2,3-b]pyridin-6-yl)urea (3.8 mg, 13%) as a light yellow solid. LCMS (ESI, m/z): [M+H]⁺=481.4. ¹H NMR (400 MHz, CD₃OD): δ 9.38 (s, 1H), 8.61 (s, 1H), 8.12 (d, J=8.4 Hz, 1H), 7.71 (s, 1H), 6.83 (d, J=8.4 Hz, 1H), 4.14 (s, 3H), 3.58-3.55 (m, 2H), 2.94-2.84 (m, 4H), 2.82-2.74 (m, 8H), 1.25-1.21 (m, 3H).

### Example S144. Compound 144

### Step 1: Synthesis of 5-bromo-2-chloro-6-methoxypyridine-3-carboxylic acid

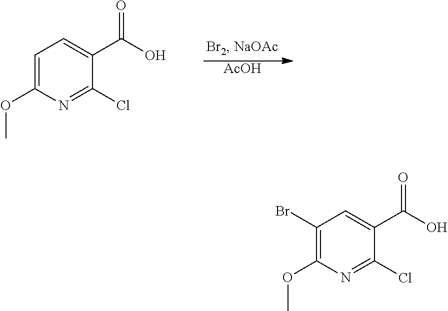

To a mixture of 2-chloro-6-methoxypyridine-3-carboxylic acid (5.0 g, 26.6 mmol) and NaOAc (4.3 g, 53.27 mmol) in AcOH (50.0 mL) was added dropwises Br₂ (17.04 g, 106.62 mmol) at room temperature. The resulting mixture was stirred at 80° C. for 16 h. After the reaction was completed, the reaction was quenched with water at room temperature and then filtered. The solid was washed with water and petroleum ether to afford 5-bromo-2-chloro-6-methoxypyridine-3-carboxylic acid (2.0 g, crude) as a white solid. LCMS (ESI, m/z): [M+H]⁺=265.9

### Step 2: Synthesis of (5-bromo-2-chloro-6-methoxypyridin-3-yl)methanol

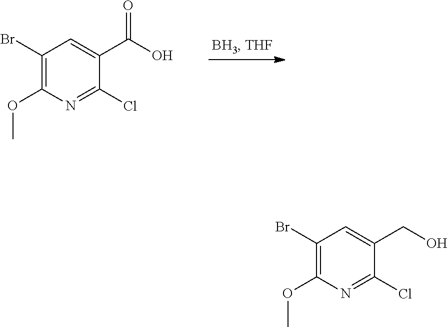

The solution of 5-bromo-2-chloro-6-methoxypyridine-3-carboxylic acid (2.0 g, 7.50 mmol) in BH₃. THE (30.0 mL, 1 mol/L) was stirred at room temperature for 2 h. After the reaction was completed, the reaction mixture was quenched with MeOH and then concentrated under reduced pressure. The residue was purified by flash column chromatography with petroleum ether/ethyl acetate (5/1, v/v) to afford (5-bromo-2-chloro-6-methoxypyridin-3-yl)methanol (1.4 g, 73%) as a white solid. LCMS (ESI, m/z): [M+H]⁺=251.9.

### Step 3: Synthesis of 5-bromo-2-chloro-6-methoxypyridine-3-carbaldehyde

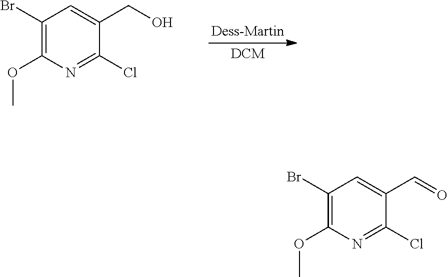

To a solution of (5-bromo-2-chloro-6-methoxypyridin-3-yl)methanol (1.4 g, 5.54 mmol) in DCM (20.0 mL) was added Dess-Martin reagent (2.3 g, 5.54 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 5-bromo-2-chloro-6-methoxypyridine-3-carbaldehyde (1.2 g, 86%) as a white solid. LCMS (ESI, m/z): [M+H]⁺=249.9.

### Step 4: Synthesis of 5-bromo-6-methoxy-1H-pyrazolo[3,4-b]pyridine

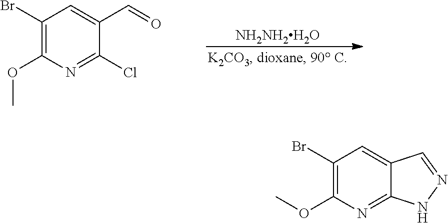

To a mixture of 5-bromo-2-chloro-6-methoxypyridine-3-carbaldehyde (400.0 mg, 1.59 mmol) and K₂CO₃ (882.8 mg, 6.38 mmol) in dioxane (5.0 mL) was added NH₂NH₂·H₂O (2.3 g, 80%) at room temperature. The resulting mixture was stirred at 90° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 5-bromo-6-methoxy-1H-pyrazolo[3,4-b]pyridine (130.0 mg, 35%) as a yellow solid. LCMS (ESI, m/z): [M+H]⁺=228.0.

### Step 5: Synthesis of 5-bromo-6-methoxy-1-[[2-(trimethylsilyl)ethoxy]methyl]pyrazolo[3,4-b]pyridine

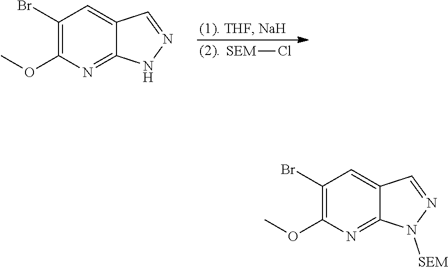 [SMILES: COc1nc2[nH]ncc2cc1Br ; C[Si](C)(C)CCOCCl ; COc1nc2c(cnn2COCC[Si](C)(C)C)cc1Br]

To a solution of 5-bromo-6-methoxy-1H-pyrazolo[3,4-b]pyridine (130.0 mg, 0.57 mmol) in THF (2.0 mL) was added NaH (27.3 mg, 60%) at 0° C. under N₂. The resulting mixture was stirred at 0° C. for 1 h under N₂. Then SEM-Cl (142.5 mg, 0.85 mmol) was added to the mixture at 0° C. The resulting mixture was stirred at 0° C. for another 2 h. After the reaction was completed, the reaction mixture was quenched with saturated NH₄Cl solution and then concentrated under reduced pressure. The residue was purified by flash column chromatography with petroleum ether/ethyl acetate (5/1, v/v) to afford 5-bromo-6-methoxy-1-[[2-(trimethylsilyl)ethoxy]methyl]pyrazolo[3,4-b]pyridine (200.0 mg, 97%) as a yellow oil. LCMS (ESI, m/z): [M+H]⁺=358.1.

### Step 6: Synthesis of 6-chloro-3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridine

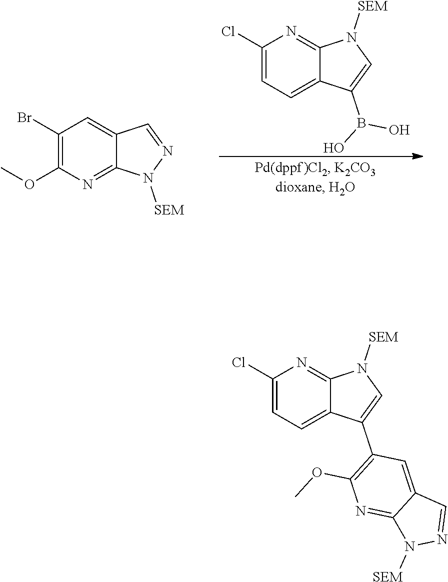 [SMILES: COc1nc2c(cnn2COCC[Si](C)(C)C)cc1Br ; C[Si](C)(C)CCOCn1cc(B(O)O)c2ccc(Cl)nc21 ; COc1nc2c(cnn2COCC[Si](C)(C)C)cc1-c1cn(COCC[Si](C)(C)C)c2nc(Cl)ccc12]

To a solution of 5-bromo-6-methoxy-1-[[2-(trimethylsilyl)ethoxy]methyl]pyrazolo[3,4-b]pyridine (200.0 mg, 0.55 mmol) in dioxane/H₂O (5.0/0.5 mL) were added 6-chloro-1-[[2-(trimethylsilyl)ethoxy]methyl]pyrrolo[2,3-b]pyridin-3-ylboronic acid (182.3 mg, 0.55 mmol), Pd(dppf)Cl₂ (40.8 mg, 0.05 mmol) and K₂CO₃ (231.4 mg, 1.67 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 6-chloro-3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridine (150.0 mg, 47%) as a yellow oil. LCMS (ESI, m/z): [M+H]⁺=560.2.

### Step 7: Synthesis of (1S,2S)-2-fluoro-N-[3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridin-6-yl]cyclopropane-1-carboxamide

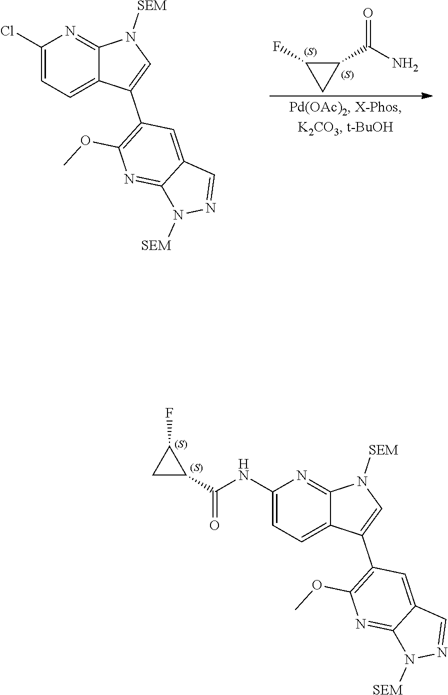

To a solution of 6-chloro-3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridine (110.0 mg, 0.19 mmol) in BuOH (5.0 mL) was added (1S,2S)-2-fluorocyclopropane-1-carboxamide (101.2 mg, 0.98 mmol), Pd(OAc)₂ (4.4 mg, 0.02 mmol), K₂CO₃ (81.4 mg, 0.58 mmol) and Xphos (18.7 mg, 0.03 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridin-6-yl]cyclopropane-1-carboxamide (65.0 mg, 52%) as a white solid. LCMS (ESI, m/z): [M+H]⁺=627.3

### Step 8: Synthesis of (1S,2S)-2-fluoro-N-(3-[6-methoxy-1H-pyrazolo[3,4-b]pyridin-5-yl]-1H-pyrrolo[2,3-b]pyridin-6-yl)cyclopropane-1-carboxamide (Compound 144)

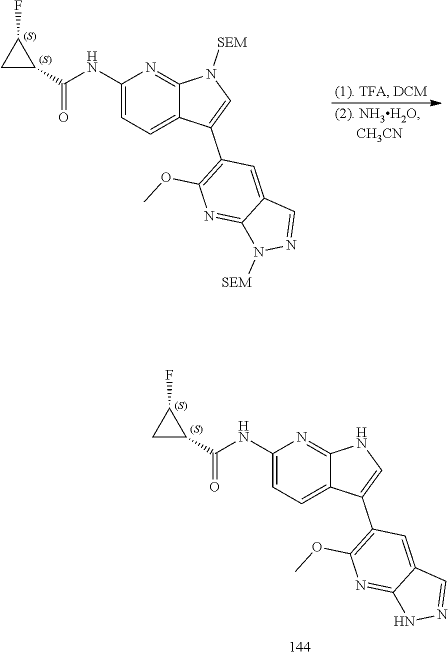

To a solution of (1S,2S)-2-fluoro-N-[3-(6-methoxy-1-[[2-(trimethylsilyl)ethoxy]methyl]pyrazolo[3,4-b]pyridin-5-yl)-1-[[2-(trimethylsilyl)ethoxy]methyl]pyrrolo[2,3-b]pyridin-6-yl]cyclopropane-1-carboxamide (110.0 mg, 0.17 mmol) in DCM (3.0 mL) was added TFA (1.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: YMC-Actus Triart C18 30×250 mm, 5 um; Mobile Phase A: Water (10 mmol/L NH₄HCO₃), Mobile Phase B: ACN; Flow rate: 60 mL/min; Gradient: 24% B to 42% B in 9 min; 254 nm) to afford (1S,2S)-2-fluoro-N-(3-[6-methoxy-1H-pyrazolo[3,4-b]pyridin-5-yl]-1H-pyrrolo[2,3-b]pyridin-6-yl) cyclopropane-1-carboxamide (6.0 mg, 9%) as a white solid. LCMS (ESI, m/z): [M+H]⁺=367.1. ¹H NMR (300 MHz, DMSO-d₆): δ 13.36 (s, 1H), 11.63 (s, 1H), 10.65 (s, 1H), 8.26 (s, 1H), 8.08-7.90 (m, 3H), 7.63 (d, J=2.4 Hz, 1H), 5.07-4.80 (m, 1H), 4.00 (s, 3H), 2.44-2.31 (m, 1H), 1.73-1.60 (m, 1H), 1.24-1.12 (m, 1H).

### Example S145. Compound 145

### Step 1: Synthesis of 4-fluoro-2-iodobenzene-1,3-diol

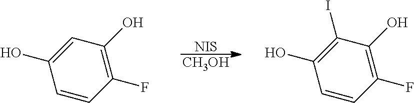

To a solution of 4-fluorobenzene-1,3-diol (10.0 g, 78.06 mmol) in CH₃OH (100.0 mL) was added NIS (8.8 g, 39.03 mmol) at 0° C. under N₂. The resulting mixture was stirred at 0° C. for 16. After the reaction was completed, the reaction was quenched with saturated Na₂S203 solution at 0° C. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (93/7, v/v) to afford 4-fluoro-2-iodobenzene-1,3-diol (9.7 g, 49%) as a white solid.

### Step 2: Synthesis of 1-fluoro-3-iodo-2,4-dimethoxybenzene

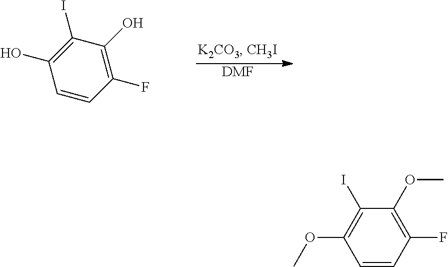

To a solution of 4-fluoro-2-iodobenzene-1,3-diol (9.7 g, 38.19 mmol) in DMF (20.0 mL) was added K₂CO₃ (15.8 g, 114.32 mmol) and CH₃I (11.9 g, 83.63 mmol) at room temperature under N₂. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (100/0, v/v) to afford 1-fluoro-3-iodo-2,4-dimethoxybenzene (5.8 g, 54%) as a white solid.

### Step 3: Synthesis of 6-chloro-3-(3-fluoro-2,6-dimethoxyphenyl)-1-[[2-(trimethylsilyl)ethoxy]methyl]pyrrolo[2,3-b]pyridine

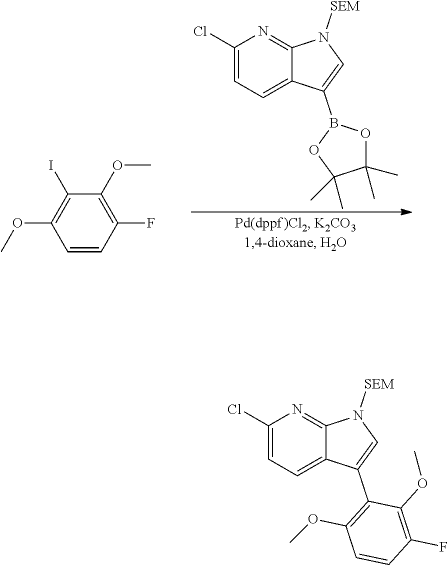 [SMILES: COc1ccc(F)c(OC)c1I ; CC1(C)OB(c2cn(COCC[Si](C)(C)C)c3nc(Cl)ccc23)OC1(C)C ; COc1ccc(F)c(OC)c1-c1cn(COCC[Si](C)(C)C)c2nc(Cl)ccc12]

To a solution of 1-fluoro-3-iodo-2,4-dimethoxybenzene (2.0 g, 7.09 mmol) in 1,4-dioxane/H₂O (5.0/1.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (3.5 g, 8.51 mmol), K₂CO₃ (2.9 g, 21.27 mmol) and Pd(dppf)Cl₂ (518.8 mg, 0.71 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (94/6, v/v) to afford 6-chloro-3-(3-fluoro-2,6-dimethoxyphenyl)-1-[[2-(trimethylsilyl)ethoxy]methyl]pyrrolo[2,3-b]pyridine (2.3 g, 74%) as a yellow oil. LCMS (ESI, m/z): [M+H]⁺=437.1.

### Step 4: Synthesis of N-(3-(3-fluoro-2,6-dimethoxyphenyl)-1-((2-(trimethylsilyl)ethoxy)methyl)-1H-pyrrolo[2,3-b]pyridin-6-yl)-1,1-diphenylmethanimine

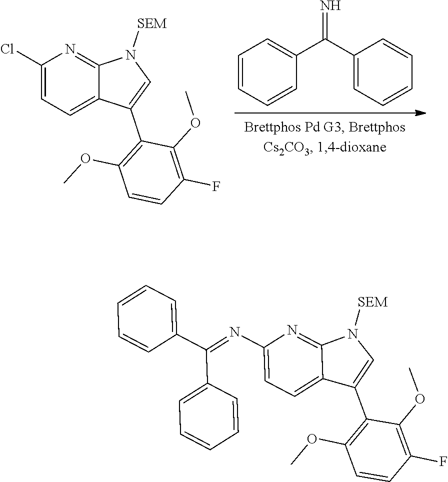 [SMILES: COc1ccc(F)c(OC)c1-c1cn(COCC[Si](C)(C)C)c2nc(Cl)ccc12 ; N=C(c1ccccc1)c1ccccc1 ; COc1ccc(F)c(OC)c1-c1cn(COCC[Si](C)(C)C)c2nc(N=C(c3ccccc3)c3ccccc3)ccc12]

To a solution of 6-chloro-3-(3-fluoro-2,6-dimethoxyphenyl)-1-[[2-(trimethylsilyl)ethoxy]methyl]pyrrolo[2,3-b]pyridine (500.0 mg, 1.14 mmol) in 1,4-dioxane (5.0 mL) was added diphenylmethanimine (622.1 mg, 3.43 mmol), Cs₂CO₃ (1.1 g, 3.43 mmol), BrettPhos (122.8 mg, 0.23 mmol) and BrettPhos Pd G3 (103.7 mg, 0.11 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (56/44, v/v) to afford N-(3-(3-fluoro-2,6-dimethoxyphenyl)-1-((2-(trimethylsilyl)ethoxy)methyl)-1H-pyrrolo[2,3-b]pyridin-6-yl)-1,1-diphenylmethanimine (200.0 mg, 30%) as a white solid. LCMS (ESI, m/z): [M+H]⁺=582.3.

### Step 5: Synthesis of 3-(3-fluoro-2,6-dimethoxyphenyl)-1-[[2-(trimethylsilyl)ethoxy]methyl]pyrrolo[2,3-b]pyridin-6-amine

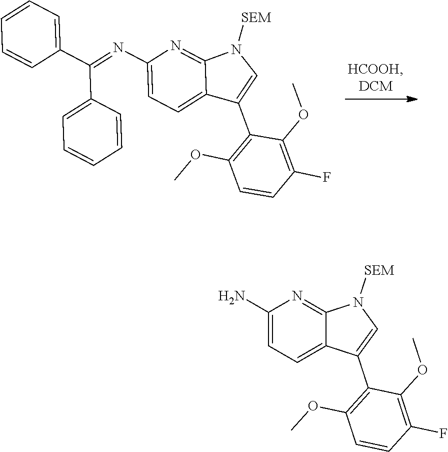 [SMILES: COc1ccc(F)c(OC)c1-c1cn(COCC[Si](C)(C)C)c2nc(N=C(c3ccccc3)c3ccccc3)ccc12 ; COc1ccc(F)c(OC)c1-c1cn(COCC[Si](C)(C)C)c2nc(N)ccc12]

To a solution of N-[3-(3-fluoro-2,6-dimethoxyphenyl)-1-[[2-(trimethylsilyl)ethoxy]methyl]pyrrolo[2,3-b]pyridin-6-yl]-1,1-diphenylmethanimine (200.0 mg, 0.34 mmol) in DCM (5.0 mL) was added HCOOH (0.5 ml) at room temperature under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (47/53, v/v) to afford 3-(3-fluoro-2,6-dimethoxyphenyl)-1-[[2-(trimethylsilyl)ethoxy]methyl]pyrrolo[2,3-b]pyridin-6-amine (100.0 mg, 69%) as a yellow oil. LCMS (ESI, m/z): [M+H]⁺=418.2.

### Step 6: Synthesis of 3-[3-(3-fluoro-2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea

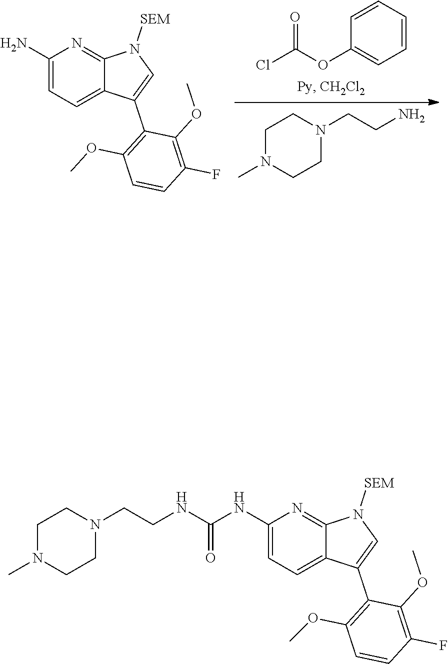

To a solution of 3-(3-fluoro-2,6-dimethoxyphenyl)-1-[[2-(trimethylsilyl)ethoxy]methyl]pyrrolo[2,3-b]pyridin-6-amine (100.0 mg, 0.24 mmol) in CH₂Cl₂ (5.0 mL) was added pyridine (75.7 mg, 0.96 mmol) and phenyl chloroformate (37.5 mg, 0.24 mmol) at 0° C. under N₂. The resulting mixture was stirred at 0° C. for 1 h. The resulting mixture was concentrated under reduced pressure. To the above mixture in pyridine (5.0 mL) was added 2-(4-methylpiperazin-1-yl)ethanamine (137.4 mg, 0.96 mmol) at 0° C. under N₂. The resulting mixture was stirred at 60° C. for another 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (88/12, v/v) to afford 3-[3-(3-fluoro-2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea (100.0 mg, 71%) as a white solid. LCMS (ESI, m/z): [M+H]⁺=587.3.

### Step 7: Synthesis of 3-[3-(3-fluoro-2,6-dimethoxyphenyl)-1H-pyrrolo[2,3-b]pyridin-6-yl]-1-[2-(4-methylpiperazin-1-yl)ethyl]urea (Compound 145)

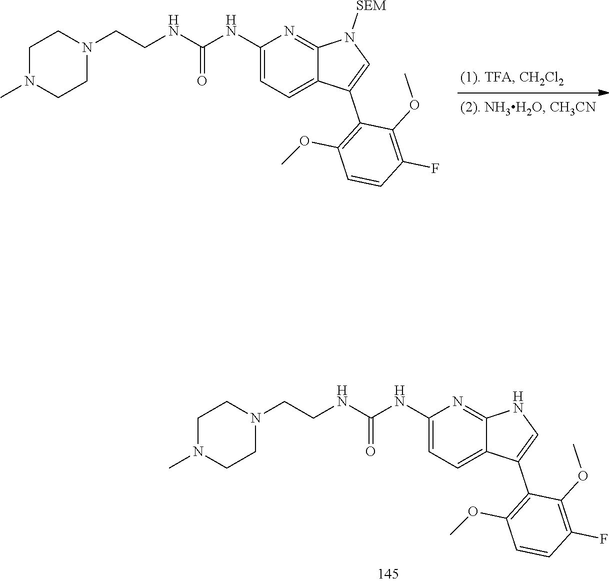 [SMILES: COc1ccc(F)c(OC)c1-c1cn(COCC[Si](C)(C)C)c2nc(NC(=O)NCCN3CCN(C)CC3)ccc12 ; COc1ccc(F)c(OC)c1-c1c[nH]c2nc(NC(=O)NCCN3CCN(C)CC3)ccc12]

To a solution of 3-[3-(3-fluoro-2,6-dimethoxyphenyl)-1-[[2-(trimethylsilyl)ethoxy]methyl]pyrrolo[2,3-b]pyridin-6-yl]-1-[2-(4-methylpiperazin-1-yl)ethyl]urea (140.0 mg, 0.24 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (2.0 mL) was added NH₃·H₂O (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XSelect CSH Fluoro Phenyl, 30×250 mm, 5 um; Mobile Phase A: Water (10 mmol/L NH₄HCO₃), Mobile Phase B: MeOH—Preparative; Flow rate: 25 mL/min; Gradient: 63% B to 87% B in 7 min; 254 nm) to afford 3-[3-(3-fluoro-2,6-dimethoxyphenyl)-1H-pyrrolo[2,3-b]pyridin-6-yl]-1-[2-(4-methylpiperazin-1-yl)ethyl]urea (27.4 mg, 25%) as a white solid. LCMS (ESI, m/z): [M+H]⁺=457.3. ¹H NMR (400 MHz, CDCl₃): δ 10.18 (s, 1H), 9.78 (s, 1H), 7.70 (d, J=8.4 Hz, 1H), 7.33 (d, J=2.4 Hz, 1H), 7.28 (s, 1H), 7.08-7.03 (m, 1H), 6.69-6.66 (m, 1H), 6.43 (d, J=8.4 Hz, 1H), 3.79 (s, 3H), 3.64-3.61 (m, 5H), 2.90-2.78 (m, 6H), 2.64-2.61 (m, 4H), 2.41 (s, 3H).

### Example S146. Compound 146

### Step 1: Synthesis of 1-benzoyl-3-(5-bromo-6-methoxypyridin-2-yl)thiourea

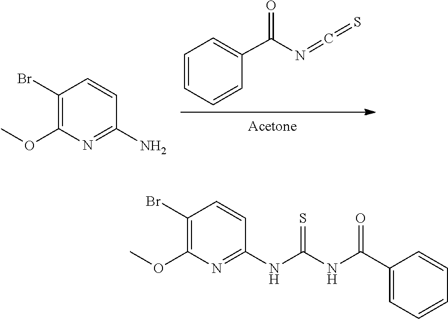

To a solution of 5-bromo-6-methoxypyridin-2-amine (3.0 g, 14.77 mmol) in acetone (40.0 mL) was added benzoyl isothiocyanate (2.9 g, 17.73 mmol) at room temperature under N₂. The resulting mixture was stirred at 60° C. for 30 min. After the reaction was completed, the resulting mixture was concentrated under reduced pressure to afford 1-benzoyl-3-(5-bromo-6-methoxypyridin-2-yl)thiourea (5.7 g, crude) as a white solid. LCMS (ESI, m/z): [M+H]⁺=366.0.

### Step 2: Synthesis of 5-bromo-6-methoxypyridin-2-ylthiourea

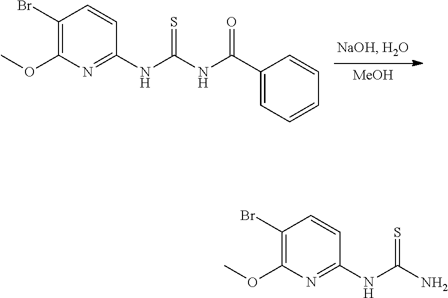 [SMILES: "MeOH" ; COc1nc(NC(=S)NC(=O)c2ccccc2)ccc1Br ; COc1nc(NC(N)=S)ccc1Br]

To a solution of 1-benzoyl-3-(5-bromo-6-methoxypyridin-2-yl)thiourea (5.2 g, crude) in MeOH/H₂O (20.0/10.0 mL) was added NaOH (1.1 g, 28.54 mmol) at room temperature under N₂. The resulting mixture was stirred at 60° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 5-bromo-6-methoxypyridin-2-ylthiourea (3.5 g, crude) as a white solid. LCMS (ESI, m/z): [M+H]⁺=262.0.

### Step 3: Synthesis of 6-bromo-5-methoxy-[1,3]thiazolo[4,5-b]pyridin-2-amine

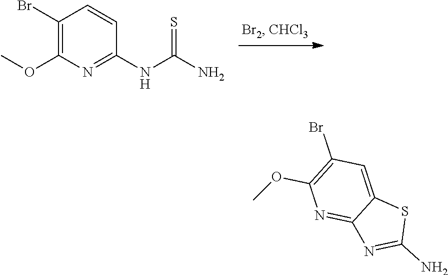

To a solution of 5-bromo-6-methoxypyridin-2-ylthiourea (1.5 g, crude) in CHCl₃ (15.0 mL) was added Br₂ (0.9 g, 5.72 mmol) at 0° C. under N₂. The resulting mixture was stirred at 60° C. for 16 h. After the reaction was completed, the reaction was quenched with sat. NH₄Cl (aq.) and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford 6-bromo-5-methoxy-[1,3]thiazolo[4,5-b]pyridin-2-amine (690.0 mg, 46%) as a white solid. LCMS (ESI, m/z): [M+H]⁺=259.9.

### Step 4: Synthesis of 6-bromo-5-methoxy-[1,3]thiazolo[4,5-b]pyridine

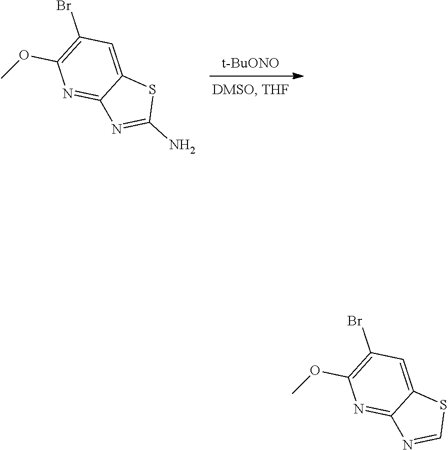

To a solution of 6-bromo-5-methoxy-[1,3]thiazolo[4,5-b]pyridin-2-amine (690.0 mg, 2.65 mmol) in THF (10.0 mL) was added DMSO (18.6 mg, 0.24 mmol) and t-BuONO (410.3 mg, 3.98 mmol) at room temperature under N₂. The resulting mixture was stirred at 30° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (92/8, v/v) to afford 6-bromo-5-methoxy-[1,3]thiazolo[4,5-b]pyridine (120.0 mg, 18%) as a white solid. LCMS (ESI, m/z): [M+H]⁺=244.9.

### Step 5: Synthesis of 6-chloro-3-[5-methoxy-[1,3]thiazolo[4,5-b]pyridin-6-yl]-1-[[2-(trimethylsilyl)ethoxy]methyl]pyrrolo[2,3-b]pyridine

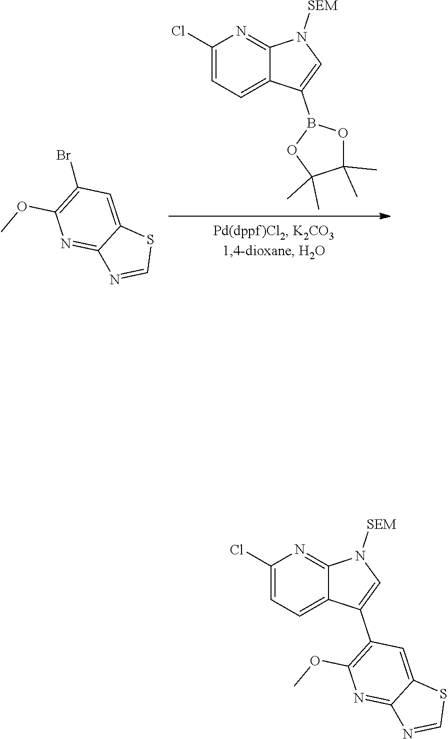 [SMILES: COc1nc2ncsc2cc1Br ; CC1(C)OB(c2cn(COCC[Si](C)(C)C)c3nc(Cl)ccc23)OC1(C)C ; COc1nc2ncsc2cc1-c1cn(COCC[Si](C)(C)C)c2nc(Cl)ccc12]

To a solution of 6-bromo-5-methoxy-[1,3]thiazolo[4,5-b]pyridine (120.0 mg, 0.49 mmol) in 1,4-dioxane/H₂O (5.0/1.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (240.2 mg, 0.58 mmol), K₂CO₃ (203.0 mg, 1.46 mmol) and Pd(dppf)Cl₂ (79.5 mg, 0.05 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford 6-chloro-3-[5-methoxy-[1,3]thiazolo[4,5-b]pyridin-6-yl]-1-[[2-(trimethylsilyl)ethoxy]methyl]pyrrolo[2,3-b]pyridine (130.0 mg, 32%) as a white solid. LCMS (ESI, m/z): [M+H]⁺=447.1.

### Step 6: Synthesis of (1S,2S)-2-fluoro-N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)cyclopropane-1-carboxamide

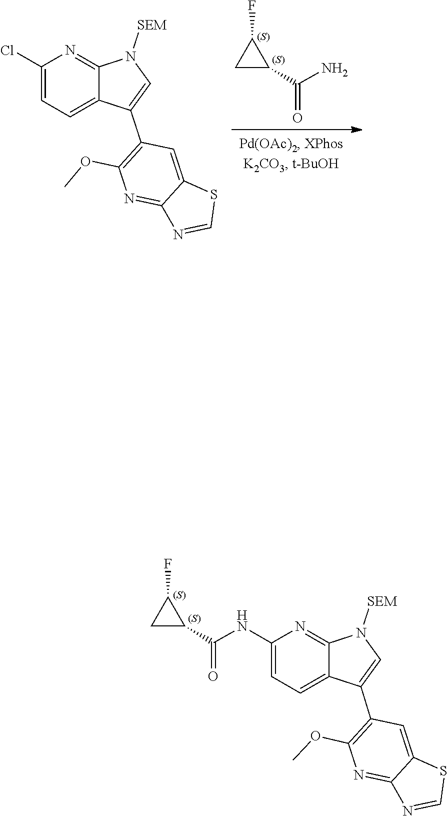

To a solution of 6-chloro-3-[5-methoxy-[1,3]thiazolo[4,5-b]pyridin-6-yl]-1-[[2-(trimethylsilyl)ethoxy]methyl]pyrrolo[2,3-b]pyridine (100.0 mg, 0.22 mmol) in t-BuOH (5.0 mL) was added (1S,2S)-2-fluorocyclopropane-1-carboxamide (115.3 mg, 1.12 mmol), XPhos (21.3 mg, 0.04 mmol), K₂CO₃ (92.7 mg, 0.61 mmol) and Pd(OAc)₂ (5.0 mg, 0.02 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (65/35, v/v) to afford (1S,2S)-2-fluoro-N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)cyclopropane-1-carboxamide (47.0 mg, 41%) as a white solid. LCMS (ESI, m/z): [M+H]⁺=514.2.

### Step 7: Synthesis of (1S,2S)-2-fluoro-N-(3-[5-methoxy-[1,3]thiazolo[4,5-b]pyridin-6-yl]-1H-pyrrolo[2,3-b]pyridin-6-yl)cyclopropane-1-carboxamide (Compound 146)

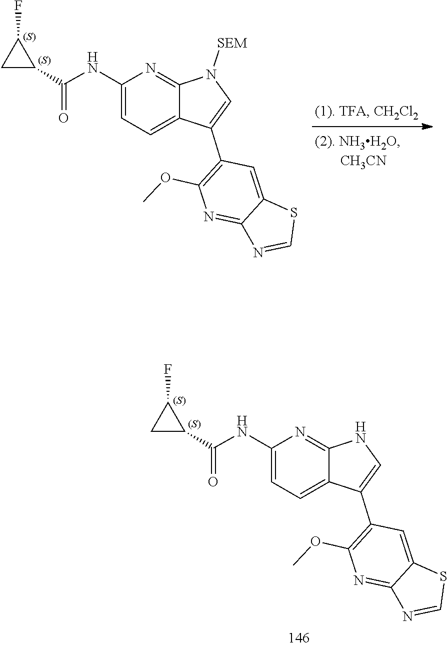

To a solution of (1S,2S)-2-fluoro-N-(3-[5-methoxy-[1,3]thiazolo[4,5-b]pyridin-6-yl]-1-[[2-(trimethylsilyl)ethoxy]methyl]pyrrolo[2,3-b]pyridin-6-yl)cyclopropane-1-carboxamide (47.0 mg, 0.09 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (3.0 mL) was added NH₃·H₂O (3 mL) at room temperature. The resulting mixture was stirred at room temperature for another 16 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 10 min; 254 nm) to afford (1S,2S)-2-fluoro-N-(3-[5-methoxy-[1,3]thiazolo[4,5-b]pyridin-6-yl]-1H-pyrrolo[2,3-b]pyridin-6-yl)cyclopropane-1-carboxamide (5.9 mg, 16%) as a white solid. LCMS (ESI, m/z): [M+H]⁺=384.1. ¹H NMR (400 MHz, DMSO-d₆): δ 11.80 (s, 1H), 10.68 (s, 1H), 9.51 (s, 1H), 8.81 (s, 1H), 8.25 (d, J=8.8 Hz, 1H), 7.95 (d, J=8.4 Hz, 1H), 7.84 (d, J=1.6 Hz, 1H), 5.04-4.83 (m, 1H), 4.06 (s, 3H), 2.26-2.23 (m, 1H), 1.72-1.63 (m, 1H), 1.24-1.12 (m, 1H).

### Example S147. Compound 147

### Step 1: Synthesis of 3-fluoro-4-iodo-5-methoxyaniline

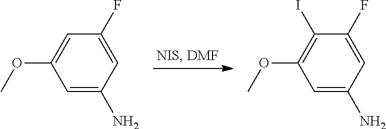

To a solution of 3-fluoro-5-methoxyaniline (2.0 g, 14.17 mmol) in DMF (20.0 mL) was added NIS (3.2 g, 14.17 mmol) at room temperature. The resulting mixture was stirred at room temperature for 0.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-fluoro-4-iodo-5-methoxyaniline (2.5 g, 66%) as a yellow solid. LCMS (ESI, m/z): [M+H]⁺=268.0.

### Step 2: Synthesis of N-((3-fluoro-4-iodo-5-methoxyphenyl)carbamothioyl)benzamide

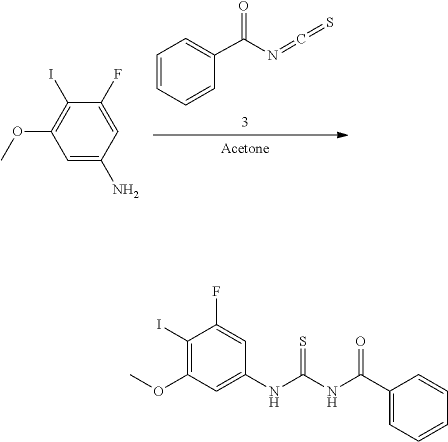

To a solution of 3-fluoro-4-iodo-5-methoxyaniline (2.5 g, 9.36 mmol) in acetone (50.0 mL) was added benzoyl isothiocyanate (1.5 g, 9.36 mmol) at room temperature. The resulting mixture was stirred at 60° C. for 30 min. After the reaction was completed, the reaction mixture was cooled to room temperature and filtered. The solid was collected and dried to afford N-((3-fluoro-4-iodo-5-methoxyphenyl)carbamothioyl)benzamide (3.8 g, crude) as a white solid. LCMS (ESI, m/z): [M+H]⁺=431.0.

### Step 3: Synthesis of 1-(3-fluoro-4-iodo-5-methoxyphenyl)thiourea

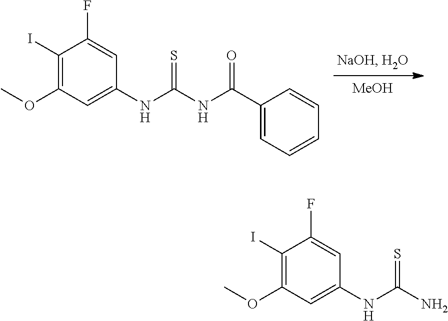 [SMILES: "MeOH" ; COc1cc(NC(=S)NC(=O)c2ccccc2)cc(F)c1I ; COc1cc(NC(N)=S)cc(F)c1I]

To a solution of N-((3-fluoro-4-iodo-5-methoxyphenyl)carbamothioyl)benzamide (3.8 g, 8.83 mmol) in MeOH/H₂O (30.0/30.0 mL) was added NaOH (0.4 g, 9.7 mmol) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 1-(3-fluoro-4-iodo-5-methoxyphenyl)thiourea (1.9 g, 65%) as a white solid. LCMS (ESI, m/z): [M+H]⁺=326.9.

### Step 4: Synthesis of 7-fluoro-6-iodo-5-methoxybenzo[d]thiazol-2-amine

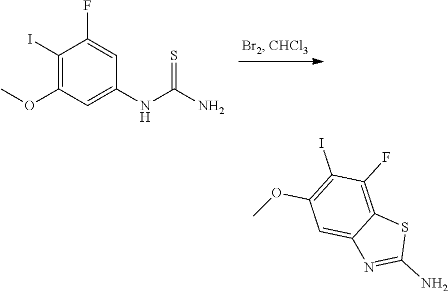

To a solution of 1-(3-fluoro-4-iodo-5-methoxyphenyl)thiourea (1.9 g, 5.83 mmol) in CHCl₃ (30.0 mL) was added dropwise Br₂ (1.0 g, 6.41 mmol) at 0° C. The resulting mixture was stirred at room temperature for 16 h. After the reaction was completed, the resulting mixture was quenched with aq. NaHSO₃ and extracted with CH₂Cl₂. The combined organic layer was washed with brine, dried over anhydrous sodium sulfate and filtered. The filtrate was concentrated under reduced pressure to afford 7-fluoro-6-iodo-5-methoxybenzo[d]thiazol-2-amine (1.3 g, crude) as a white solid. LCMS (ESI, m/z): [M+H]⁺=324.9.

### Step 5: Synthesis of 7-fluoro-6-iodo-5-methoxybenzo[d]thiazole

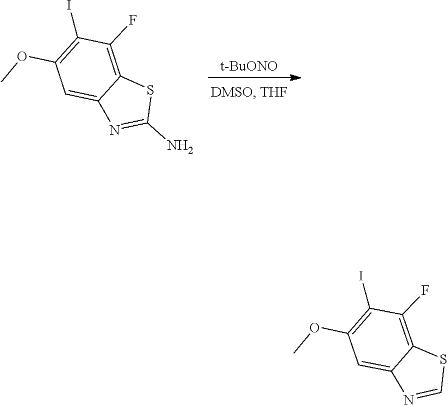

To a solution of 7-fluoro-6-iodo-5-methoxybenzo[d]thiazol-2-amine (1.3 g, 4.01 mmol) in THE (30.0 mL) was added t-BuONO (0.6 g, 6.02 mmol) and DMSO (25.1 mg, 0.32 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 7-fluoro-6-iodo-5-methoxybenzo[d]thiazole (1.2 g, crude) as a yellow solid. LCMS (ESI, m/z): [M+H]⁺=309.9.

### Step 6: Synthesis of 6-(6-chloro-1-((2-(trimethylsilyl)ethoxy)methyl)-1H-pyrrolo[2,3-b]pyridin-3-yl)-7-fluoro-5-methoxybenzo[d]thiazole

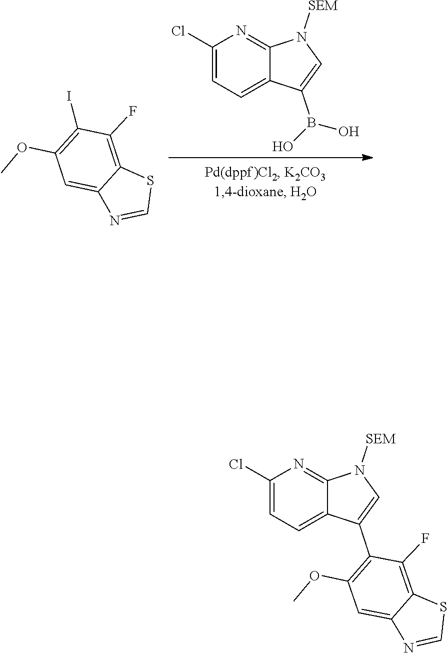 [SMILES: COc1cc2ncsc2c(F)c1I ; C[Si](C)(C)CCOCn1cc(B(O)O)c2ccc(Cl)nc21 ; COc1cc2ncsc2c(F)c1-c1cn(COCC[Si](C)(C)C)c2nc(Cl)ccc12]

To a solution of 7-fluoro-6-iodo-5-methoxybenzo[d]thiazole (300.0 mg, 0.97 mmol) in 1,4-dioxane/H₂O (16.0/4.0 mL) was added (6-chloro-1-((2-(trimethylsilyl)ethoxy)methyl)-1H-pyrrolo[2,3-b]pyridin-3-yl)boronic acid (317.0 mg, 0.97 mmol), K₂CO₃ (402.4 mg, 2.91 mmol) and Pd(dppf)Cl₂ (71.0 mg, 0.10 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-(6-chloro-1-((2-(trimethylsilyl)ethoxy)methyl)-1H-pyrrolo[2,3-b]pyridin-3-yl)-7-fluoro-5-methoxybenzo[d]thiazole (270.0 mg, 59%) as a yellow oil. LCMS (ESI, m/z): [M+H]⁺=464.1.

### Step 7: Synthesis of (1S,2S)-2-fluoro-N-(3-(7-fluoro-5-methoxybenzo[d]thiazol-6-yl)-1-((2-(trimethylsilyl)ethoxy)methyl)-1H-pyrrolo[2,3-b]pyridin-6-yl)cyclopropane-1-carboxamide

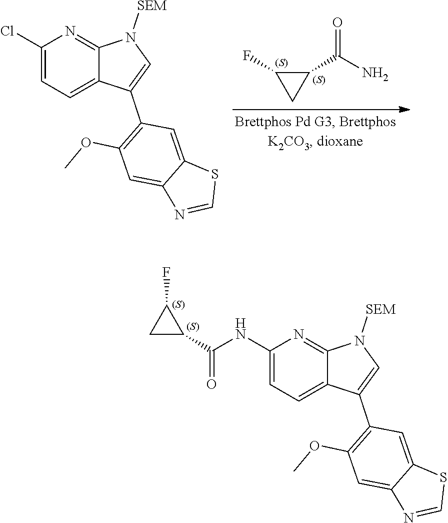

To a solution of 6-(6-chloro-1-((2-(trimethylsilyl)ethoxy)methyl)-1H-pyrrolo[2,3-b]pyridin-3-yl)-7-fluoro-5-methoxybenzo[d]thiazole (210.0 mg, 0.45 mmol) in dioxane (10.0 mL) was added (1S,2S)-2-fluorocyclopropane-1-carboxamide (233.3 mg, 2.27 mmol), K₂CO₃ (187.6 mg, 1.36 mmol), Brettphos (48.6 mg, 0.09 mmol) and BrettPhos Pd G3 (41.0 mg, 0.05 mmol) at room temperature under N₂. The resulting mixture was stirred at 100° C. for 4 h under N₂. After the reaction was completed, the mixture was evaporated in vacuo. The residue was purified by flash column chromatography with petroleum ether/ethyl acetate (1/1, v/v) to afford (1S,2S)-2-fluoro-N-(3-(7-fluoro-5-methoxybenzo[d]thiazol-6-yl)-1-((2-(trimethylsilyl)ethoxy)methyl)-1H-pyrrolo[2,3-b]pyridin-6-yl)cyclopropane-1-carboxamide (160.0 mg, 66%) as a yellow solid. LCMS (ESI, m/z): [M+H]⁺=531.2.

### Step 8: Synthesis of (1S,2S)-2-fluoro-N-(3-(7-fluoro-5-methoxybenzo[d]thiazol-6-yl)-1H-pyrrolo[2,3-b]pyridin-6-yl)cyclopropane-1-carboxamide (Compound 147)

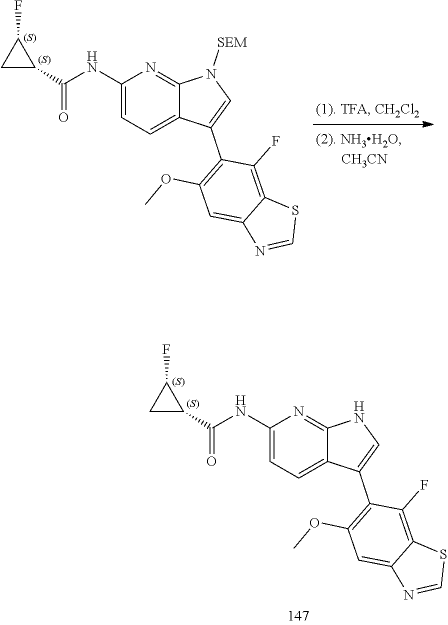

To a solution of (1S,2S)-2-fluoro-N-(3-(7-fluoro-5-methoxybenzo[d]thiazol-6-yl)-1-((2-(trimethylsilyl)ethoxy)methyl)-1H-pyrrolo[2,3-b]pyridin-6-yl)cyclopropane-1-carboxamide (140.0 mg, 0.26 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (5.0 mL) was added NH₃·H₂O (5.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column 30×150 mm, 5 um; Mobile Phase A: Water (10 mmol/L NH₄HCO₃), Mobile Phase B: ACN; Flow rate: 60 mL/min; Gradient: 23% to 53% in 10 min; 254 nm) to afford (1S,2S)-2-fluoro-N-(3-(7-fluoro-5-methoxybenzo[d]thiazol-6-yl)-1H-pyrrolo[2,3-b]pyridin-6-yl)cyclopropane-1-carboxamide (33.5 mg, 31%) as a white solid. LCMS (ESI, m/z): [M+H]⁺=401.2. ¹H NMR (300 MHz, DMSO-d₆): δ 11.80 (s, 1H), 10.68 (s, 1H), 9.45 (s, 1H), 7.90 (d, J=8.4 Hz, 1H), 7.77-7.70 (m, 2H), 7.58 (s, 1H), 5.05-4.82 (m, 1H), 3.90 (s, 3H), 2.28-2.19 (m, 1H), 1.70-1.62 (m, 1H), 1.22-1.11 (m, 1H).

### Example S148. Compound 148 and Compound 149

### Step 1: Synthesis of 3-bromo-2-(cyclopropylmethoxy)pyridine

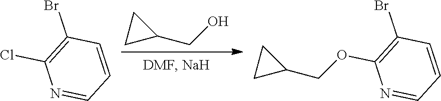

To a solution of cyclopropylmethanol (2.1 g, 28.58 mmol) in DMF (50.0 mL) was added NaH (1.9 g, 60%) at 0° C. under N₂. The resulting mixture was stirred at 0° C. for 1 h under N₂. Then 3-bromo-2-chloropyridine (5.0 g, 25.98 mmol) was added to the mixture at 0° C. under N₂. The resulting mixture was stirred at 70° C. for another 6 h. After the reaction was completed, the reaction mixture was quenched with H₂O at 0° C. and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-bromo-2-(cyclopropylmethoxy)pyridine (3.0 g, 50%) as a colorless oil. LCMS (ESI, m/z): [M+H]⁺=228.0.

### Step 2: Synthesis of 6-chloro-3-(2-(cyclopropylmethoxy)pyridin-3-yl)-1-((2-(trimethylsilyl)ethoxy)methyl)-1H-pyrrolo[2,3-b]pyridine

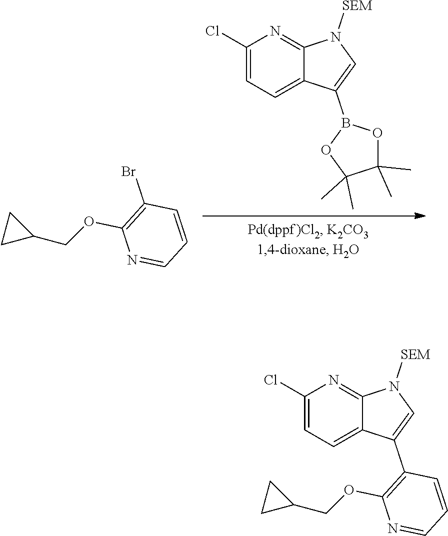 [SMILES: Brc1cccnc1OCC1CC1 ; CC1(C)OB(c2cn(COCC[Si](C)(C)C)c3nc(Cl)ccc23)OC1(C)C ; C[Si](C)(C)CCOCn1cc(-c2cccnc2OCC2CC2)c2ccc(Cl)nc21]

To a solution of 3-bromo-2-(cyclopropylmethoxy)pyridine (500.0 mg, 2.19 mmol) in 1,4-dioxane/H₂O (16.0/4.0 mL) was added 6-chloro-3-(4,4,5,5-tetramethyl-1,3,2-dioxaborolan-2-yl)-1-((2-(trimethylsilyl)ethoxy)methyl)-1H-pyrrolo[2,3-b]pyridine (896.2 mg, 2.19 mmol), K₂CO₃ (908.9 mg, 6.58 mmol) and Pd(dppf)Cl₂ (160.4 mg, 0.22 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 6-chloro-3-(2-(cyclopropylmethoxy)pyridin-3-yl)-1-((2-(trimethylsilyl)ethoxy)methyl)-1H-pyrrolo[2,3-b]pyridine (500.0 mg, 53%) as a yellow oil. LCMS (ESI, m/z): [M+H]⁺=430.2.

### Step 3: Synthesis of (1S,2S)—N-(3-(2-(cyclopropylmethoxy)pyridin-3-yl)-1-((2-(trimethylsilyl)ethoxy)methyl)-1H-pyrrolo[2,3-b]pyridin-6-yl)-2-fluorocyclopropane-1-carboxamide

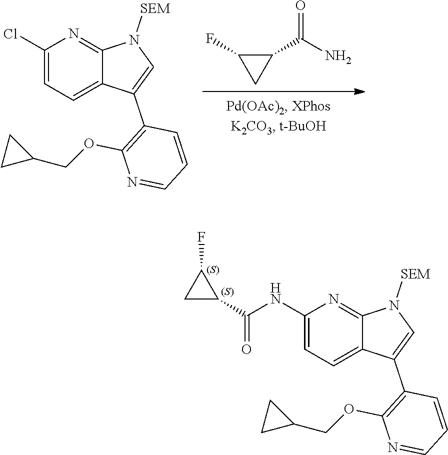

To a solution of 6-chloro-3-(2-(cyclopropylmethoxy)pyridin-3-yl)-1-((2-(trimethylsilyl)ethoxy)methyl)-1H-pyrrolo[2,3-b]pyridine (400.0 mg, 0.93 mmol) in t-BuOH (20.0 mL) was added (1S,2S)-2-fluorocyclopropane-1-carboxamide (479.5 mg, 4.65 mmol), K₂CO₃ (385.7 mg, 2.79 mmol), Pd(OAc)₂ (20.9 mg, 0.09 mmol) and XPhos (88.7 mg, 0.19 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford (1S,2S)—N-(3-(2-(cyclopropylmethoxy)pyridin-3-yl)-1-((2-(trimethylsilyl)ethoxy)methyl)-1H-pyrrolo[2,3-b]pyridin-6-yl)-2-fluorocyclopropane-1-carboxamide (160.0 mg, 34%) as a yellow oil. LCMS (ESI, m/z): [M+H]⁺=497.2.

### Step 4: Synthesis of (1S,2S)—N-(3-(2-(cyclopropylmethoxy)pyridin-3-yl)-1H-pyrrolo[2,3-b]pyridin-6-yl)-2-fluorocyclopropane-1-carboxamide and (1R,2S)—N-(3-(2-(cyclopropylmethoxy)pyridin-3-yl)-1H-pyrrolo[2,3-b]pyridin-6-yl)-2-fluorocyclopropane-1-carboxamide (Compound 148 and Compound 149)

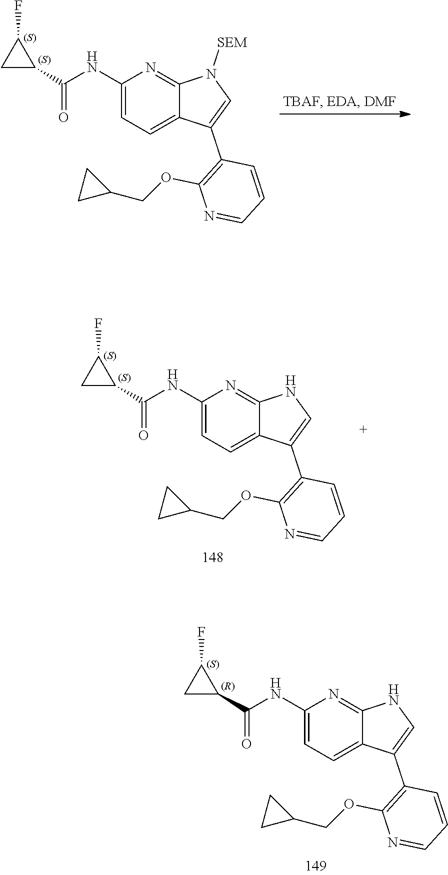

To a solution of (1S,2S)—N-(3-(2-(cyclopropylmethoxy)pyridin-3-yl)-1-((2-(trimethylsilyl)ethoxy)methyl)-1H-pyrrolo[2,3-b]pyridin-6-yl)-2-fluorocyclopropane-1-carboxamide (150.0 mg, 0.18 mmol) in DMF (5.0 mL) was added ethylenediamine (90.8 mg, 1.51 mmol) and TBAF (236.9 mg, 0.91 mmol) at room temperature. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 44% to 54% in 8 min; 254 nm) to afford N-(3-(2-(cyclopropylmethoxy)pyridin-3-yl)-1H-pyrrolo[2,3-b]pyridin-6-yl)-2-fluorocyclopropane-1-carboxamide Enantiomer 1 (5.2 mg, 4%, retention time: 7.38 minutes) as a white solid and N-(3-(2-(cyclopropylmethoxy)pyridin-3-yl)-1H-pyrrolo[2,3-b]pyridin-6-yl)-2-fluorocyclopropane-1-carboxamide Enantiomer 2 (10.2 mg, 9%, retention time, 7.79 minutes) as a white solid. The absolute stereochemistry of Enantiomers 1 and 2 was not assigned. The two enantiomeric structures that could be obtained from chiral separation of the enantiomeric mixture as described above are shown as Compounds 148 and 149 in Table 1.

N-(3-(2-(cyclopropylmethoxy)pyridin-3-yl)-1H-pyrrolo[2,3-b]pyridin-6-yl)-2-fluorocyclopropane-1-carboxamide Enantiomer 1: LCMS (ESI, m/z): [M+H]⁺=367.2. ¹H NMR (300 MHz, DMSO-d₆): δ 11.73 (s, 1H), 10.69 (s, 1H), 8.21 (d, J=8.7 Hz, 1H), 8.05-7.92 (m, 3H), 7.80 (s, 1H), 7.08-7.04 (m, 1H), 5.04-4.82 (m, 1H), 4.22 (d, J=7.2 Hz, 2H), 2.26-2.22 (m, 1H), 1.78-1.55 (m, 1H), 1.31-1.13 (m, 2H), 0.59-0.53 (m, 2H), 0.39-0.34 (m, 2H).

N-(3-(2-(cyclopropylmethoxy)pyridin-3-yl)-1H-pyrrolo[2,3-b]pyridin-6-yl)-2-fluorocyclopropane-1-carboxamide Enantiomer 2: LCMS (ESI, m/z): [M+H]⁺=367.2. ¹H NMR (300 MHz, DMSO-d₆): δ 11.75 (s, 1H), 10.81 (s, 1H), 8.20 (d, J=8.7 Hz, 1H), 8.05-7.98 (m, 2H), 7.89 (d, J=8.7 Hz, 1H), 7.80 (d, J=2.4 Hz, 1H), 7.07-7.03 (m, 1H), 5.02-4.78 (m, 1H), 4.21 (d, J=7.2 Hz, 2H), 2.69-2.55 (m, 1H), 1.55-1.48 (m, 1H), 1.29-1.22 (m, 2H), 0.57-0.52 (m, 2H), 0.38-0.35 (m, 2H).

### Example S149. Compound 150

### Step 1: Synthesis of 1-benzoyl-3-(3-bromo-2-methoxyphenyl)thiourea

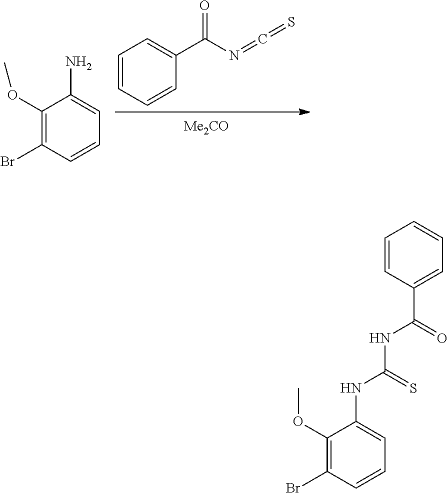

To a solution of 3-bromo-2-methoxyaniline (5.0 g, 24.75 mmol) in acetone (125.0 mL) was added benzoyl isothiocyanate (4.0 g, 24.76 mmol) at room temperature. The resulting mixture was stirred at 60° C. for 0.5 h. After the reaction was completed. The resulting mixture was concentrated under reduced pressure. The residue were washed with ethyl ether and then filtered. The solid was collected and dried to afford 1-benzoyl-3-(3-bromo-2-methoxyphenyl)thiourea (7.8 g, crude) as a light yellow solid. LCMS (ESI, m/z): [M+H]⁺=365.0.

### Step 2: Synthesis of 3-bromo-2-methoxyphenylthiourea

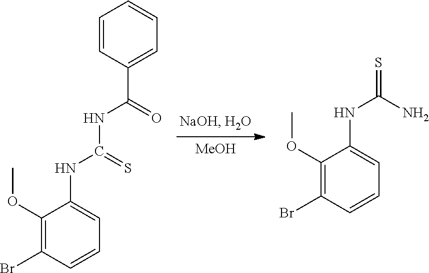 [SMILES: "MeOH" ; COc1c(Br)cccc1NC(=S)NC(=O)c1ccccc1 ; COc1c(Br)cccc1NC(N)=S]

To a solution of 1-benzoyl-3-(3-bromo-2-methoxyphenyl)thiourea (7.8 g, crude) in MeOH/H₂O (130.0 mL/26.0 mL) was added NaOH (4.3 g, 106.76 mmol) at room temperature. The resulting mixture was stirred at 80° C. for 1 h. After the reaction was completed, the resulting mixture was concentrated under reduced pressure. The residue was diluted with H₂O and extracted with ethyl acetate. The combined organic layer was washed with brine, dried over anhydrous sodium sulfate and filtered. The filtrate was concentrated under reduced pressure to afford 3-bromo-2-methoxyphenylthiourea (5.0 g, crude) as a light pink solid. LCMS (ESI, m/z): [M+H]⁺=261.0

### Step 3: Synthesis of 5-bromo-4-methoxy-1,3-benzothiazol-2-amine

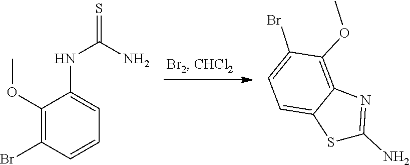

To a solution of 3-bromo-2-methoxyphenylthiourea (5.0 g, 19.15 mmol) in CHCl₃ (300.0 mL) was added dropwise Br₂ (3.7 g, 22.98 mmol) at 0° C. under N₂. The resulting mixture was stirred at room temperature for 8 h. After the reaction was completed, the reaction mixture was quenched with NaHSO₃ (aq.) at 0° C. The resulting mixture was diluted with H₂O and extracted with CH₂Cl₂. The combined organic layer was washed with brine, dried over anhydrous sodium sulfate and filtered. The filtrate was concentrated under reduced pressure. The residue was purified by reverse phase flash column chromatography with acetonitrile/water (1/1, v/v) to afford 5-bromo-4-methoxy-1,3-benzothiazol-2-amine (4.0 g, 80%) as a light brown solid. LCMS (ESI, m/z): [M+H]⁺=258.9.

### Step 4: Synthesis of 5-bromo-4-methoxy-1,3-benzothiazole

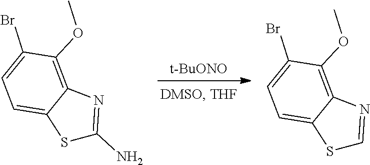

To a solution of 5-bromo-4-methoxy-1,3-benzothiazol-2-amine (4.0 g, 15.45 mmol) in THE (60.0 mL) was added tert-butyl nitrite (2.4 g, 23.16 mmol) and DMSO (96.5 mg, 1.24 mmol) at room temperature. The resulting mixture was stirred at room temperature for 2 h. After the reaction was completed, the resulting mixture was concentrated under reduced pressure. The residue was purified by flash column chromatography with petroleum ether/ethyl acetate (7/1, v/v) to afford 5-bromo-4-methoxy-1,3-benzothiazole (1.6 g, 42%) as a brown solid. LCMS (ESI, m/z): [M+H]⁺=243.9.

### Step 5: Synthesis of 5-(6-chloro-1-[[2-(trimethylsilyl)ethoxy]methyl]pyrrolo[2,3-b]pyridin-3-yl)-4-methoxy-1,3-benzothiazole

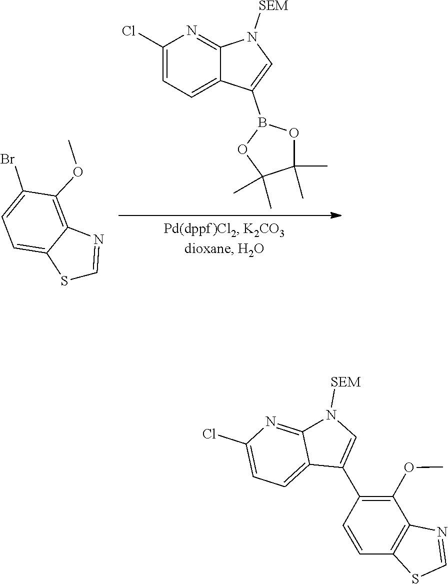 [SMILES: COc1c(Br)ccc2scnc12 ; CC1(C)OB(c2cn(COCC[Si](C)(C)C)c3nc(Cl)ccc23)OC1(C)C ; COc1c(-c2cn(COCC[Si](C)(C)C)c3nc(Cl)ccc23)ccc2scnc12]

To a solution of 5-bromo-4-methoxy-1,3-benzothiazole (124.1 mg, 0.51 mmol) in 1,4-dioxane/H₂O (10.0 mL/2.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (250.0 mg, 0.61 mmol), K₂CO₃ (140.5 mg, 1.02 mmol) and Pd(dppf)Cl₂ (37.2 mg, 0.05 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 5-(6-chloro-1-[[2-(trimethylsilyl)ethoxy]methyl]pyrrolo[2,3-b]pyridin-3-yl)-4-methoxy-1,3-benzothiazole (220.0 mg, 97%) as a yellow oil. LCMS (ESI, m/z): [M+H]⁺=446.1.

### Step 6: Synthesis of (1S,2S)-2-fluoro-N-[3-(4-methoxy-1,3-benzothiazol-5-yl)-1-[[2-(trimethylsilyl)ethoxy]methyl]pyrrolo[2,3-b]pyridin-6-yl]cyclopropane-1-carboxamide

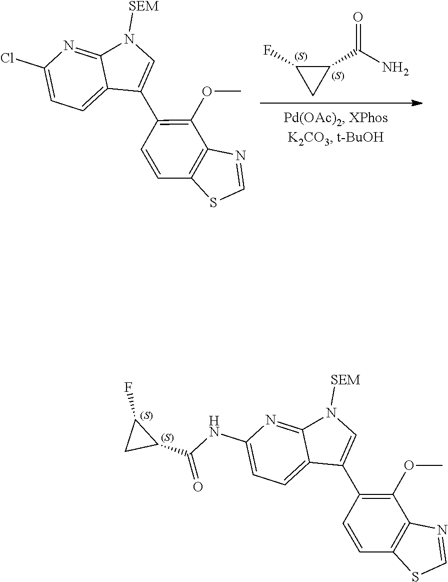

To a solution of 5-(6-chloro-1-[[2-(trimethylsilyl)ethoxy]methyl]pyrrolo[2,3-b]pyridin-3-yl)-4-methoxy-1,3-benzothiazole (190.0 mg, 0.43 mmol) in t-BuOH (20.0 mL) was added (1S,2S)-2-fluorocyclopropane-1-carboxamide (219.6 mg, 2.13 mmol), XPhos (40.6 mg, 0.09 mmol), K₂CO₃ (176.6 mg, 1.28 mmol) and Pd(OAc)₂ (9.6 mg, 0.04 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford (1S,2S)-2-fluoro-N-[3-(4-methoxy-1,3-benzothiazol-5-yl)-1-[[2-(trimethylsilyl)ethoxy]methyl]pyrrolo[2,3-b]pyridin-6-yl]cyclopropane-1-carboxamide (114.0 mg, 52%) as a yellow oil. LCMS (ESI, m/z): [M+H]⁺=513.2.

### Step 7: Synthesis of (1S,2S)-2-fluoro-N-[3-(4-methoxy-1,3-benzothiazol-5-yl)-1H-pyrrolo[2,3-b]pyridin-6-yl]cyclopropane-1-carboxamide (Compound 150)

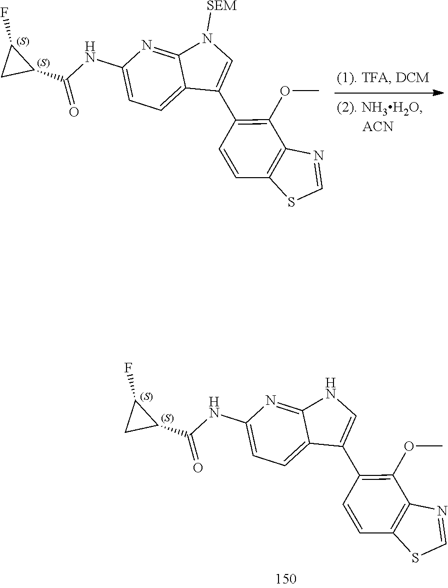

To a solution of (1S,2S)-2-fluoro-N-[3-(4-methoxy-1,3-benzothiazol-5-yl)-1-[[2-(trimethylsilyl)ethoxy]methyl]pyrrolo[2,3-b]pyridin-6-yl]cyclopropane-1-carboxamide (114.0 mg, 0.22 mmol) in CH₂Cl₂ (6.0 mL) was added TFA (6.0 mL) at room temperature. The resulting mixture was stirred at room temperature for 3 h. The resulting mixture was concentrated under reduced pressure. To the residue in CH₃CN (6.0 mL) was added NH₃·H₂O (6.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 38% B to 47% B in 8 min; 254/220 nm) to afford (1S,2S)-2-fluoro-N-[3-(4-methoxy-1,3-benzothiazol-5-yl)-1H-pyrrolo[2,3-b]pyridin-6-yl]cyclopropane-1-carboxamide (16.7 mg, 19%) as a white solid. LCMS (ESI, m/z): [M+H]⁺=383.2. ¹H NMR (400 MHz, DMSO-d₆): δ 11.69 (s, 1H), 10.65 (s, 1H), 9.37 (s, 1H), 8.08 (d, J=8.4 Hz, 1H), 7.91 (d, J=8.4 Hz, 2H), 7.71-7.67 (m, 2H), 5.01-4.84 (m, 1H), 4.05 (s, 3H), 2.28-2.19 (m, 1H), 1.70-1.62 (m, 1H), 1.23-1.17 (m, 1H).

### Example S150. Compound 151 and Compound 152

### Step 1: Synthesis of tert-butyl 4-[[trans-2-[[3-(2,6-dimethoxyphenyl)-1-[[2-(trimethylsilyl)ethoxy]methyl]pyrrolo[2,3-b]pyridin-6-yl]carbamoyl]cyclopropyl]methyl]piperazine-1-carboxylate

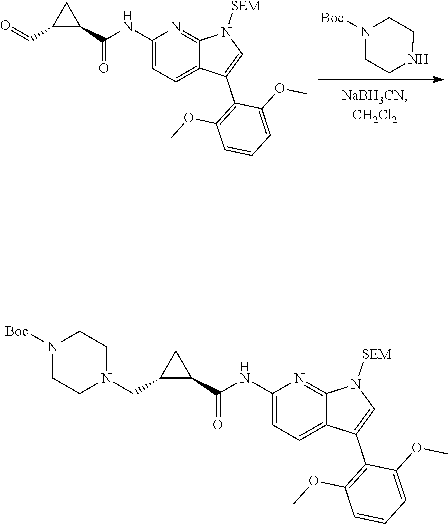 [SMILES: COc1cccc(OC)c1-c1cn(COCC[Si](C)(C)C)c2nc(NC(=O)[C@@H]3C[C@H]3C=O)ccc12 ; CC(C)(C)OC(=O)N1CCNCC1 ; COc1cccc(OC)c1-c1cn(COCC[Si](C)(C)C)c2nc(NC(=O)[C@@H]3C[C@H]3CN3CCN(C(=O)OC(C)(C)C)CC3)ccc12]

To a solution of trans-N-(3-(2,6-dimethoxyphenyl)-1-((2-(trimethylsilyl)ethoxy)methyl)-1H-pyrrolo[2,3-b]pyridin-6-yl)-2-formylcyclopropanecarboxamide (260.0 mg, 0.53 mmol) in DCM (26.0 mL) were added tert-butyl piperazine-1-carboxylate (293.1 mg, 1.57 mmol) and NaBH₃CN (98.9 mg, 1.57 mmol) at room temperature. The resulting mixture was stirred at room temperature for 2 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford tert-butyl 4-[[trans-2-[[3-(2,6-dimethoxyphenyl)-1-[[2-(trimethylsilyl)ethoxy]methyl]pyrrolo[2,3-b]pyridin-6-yl]carbamoyl]cyclopropyl]methyl]piperazine-1-carboxylate (180.0 mg, 52%) as a white solid. LCMS (ESI, m/z): [M+H]⁺=666.4.

### Step 2: Synthesis of trans-N-[3-(2,6-dimethoxyphenyl)-1H-pyrrolo[2,3-b]pyridin-6-yl]-2-(piperazin-1-ylmethyl)cyclopropane-1-carboxamide

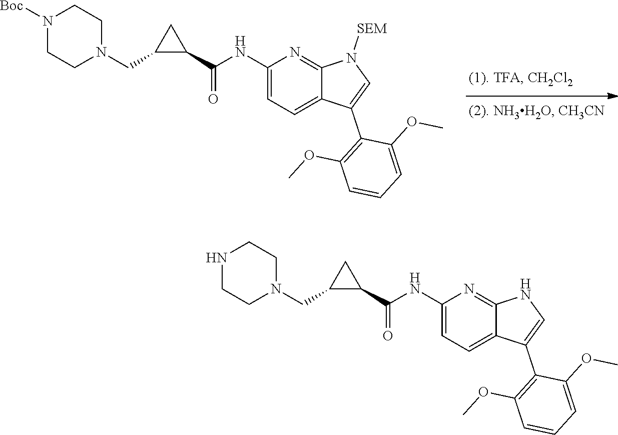 [SMILES: COc1cccc(OC)c1-c1cn(COCC[Si](C)(C)C)c2nc(NC(=O)[C@@H]3C[C@H]3CN3CCN(C(=O)OC(C)(C)C)CC3)ccc12 ; COc1cccc(OC)c1-c1c[nH]c2nc(NC(=O)[C@@H]3C[C@H]3CN3CCNCC3)ccc12]

To a solution of tert-butyl 4-[[trans-2-[[3-(2,6-dimethoxyphenyl)-1-[[2-(trimethylsilyl)ethoxy]methyl]pyrrolo[2,3-b]pyridin-6-yl]carbamoyl]cyclopropyl]methyl]piperazine-1-carboxylate (350.0 mg, 0.53 mmol) in CH₂Cl₂ (10.0 mL) was added TFA (10.0 mL) at room temperature. The resulting mixture was stirred at room temperature for 3 h. The resulting mixture was concentrated under reduced pressure. To the residue in CH₃CN (20.0 mL) was added NH₃·H₂O (20.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 43% B in 9 min; 254 nm) to afford trans-N-[3-(2,6-dimethoxyphenyl)-1H-pyrrolo[2,3-b]pyridin-6-yl]-2-(piperazin-1-ylmethyl)cyclopropane-1-carboxamide (80.0 mg, 39%) as a white solid. LCMS (ESI, m/z): [M+H]⁺=436.2.

### Step 3: Synthesis of (1R,2R)—N-[3-(2,6-dimethoxyphenyl)-1H-pyrrolo[2,3-b]pyridin-6-yl]-2-(piperazin-1-ylmethyl)cyclopropane-1-carboxamide and (1S,2S)—N-[3-(2,6-dimethoxyphenyl)-1H-pyrrolo[2,3-b]pyridin-6-yl]-2-(piperazin-1-ylmethyl)cyclopropane-1-carboxamide (Compound 151 and Compound 152)

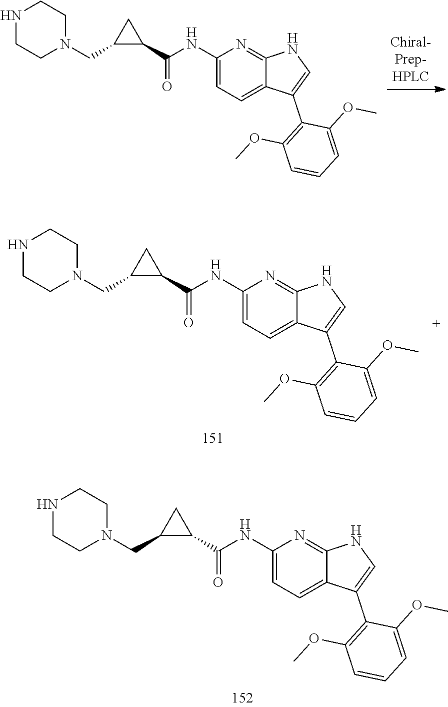 [SMILES: COc1cccc(OC)c1-c1c[nH]c2nc(NC(=O)[C@@H]3C[C@H]3CN3CCNCC3)ccc12 ; COc1cccc(OC)c1-c1c[nH]c2nc(NC(=O)[C@@H]3C[C@H]3CN3CCNCC3)ccc12 ; COc1cccc(OC)c1-c1c[nH]c2nc(NC(=O)[C@H]3C[C@@H]3CN3CCNCC3)ccc12]

The racemic trans-N-[3-(2,6-dimethoxyphenyl)-1H-pyrrolo[2,3-b]pyridin-6-yl]-2-(piperazin-1-ylmethyl)cyclopropane-1-carboxamide (80.0 mg, 0.18 mmol) was separated by Prep-Chiral-HPLC with the following conditions (Column: CHIRALPAK IE, 2×25 cm, 5 um; Mobile Phase A: MTBE (0.5% 2M NH₃-MeOH)—HPLC, Mobile Phase B: MeOH—HPLC; Flow rate: 20 mL/min; Gradient: 15% B to 15% B in 13 min; 220/254 nm) to afford N-[3-(2,6-dimethoxyphenyl)-1H-pyrrolo[2,3-b]pyridin-6-yl]-2-(piperazin-1-ylmethyl)cyclopropane-1-carboxamide Enantiomer 1 (12.4 mg, 15%, Retention time 1: 7.557 min) as a white solid and N-[3-(2,6-dimethoxyphenyl)-1H-pyrrolo[2,3-b]pyridin-6-yl]-2-(piperazin-1-ylmethyl)cyclopropane-1-carboxamide Enantiomer 2 (12.5 mg, 15%, Retention time 2: 9.866 min) as a white solid. The absolute stereochemistry of Enantiomers 1 and 2 was not assigned. The two enantiomeric structures that could be obtained from chiral separation of the enantiomeric mixture as described above are shown as Compounds 151 and 152 in Table 1.

N-[3-(2,6-dimethoxyphenyl)-1H-pyrrolo[2,3-b]pyridin-6-yl]-2-(piperazin-1-ylmethyl)cyclopropane-1-carboxamide Enantiomer 1: LCMS (ESI, m/z): [M+H]⁺=436.3. ¹H NMR (400 MHz, DMSO-d₆): δ 11.40 (s, 1H), 10.51 (s, 1H), 7.81 (d, J=8.8 Hz, 1H), 7.52 (d, J=8.4 Hz, 1H), 7.29-7.25 (m, 2H), 6.74 (d, J=8.4 Hz, 2H), 3.68 (s, 6H), 2.73-2.67 (m, 4H), 2.38-2.32 (m, 5H), 2.22-2.18 (m, 1H), 1.89-1.87 (m, 1H), 1.35-1.31 (m, 1H), 1.04-0.98 (m, 1H), 0.78-0.61 (m, 1H).

N-[3-(2,6-dimethoxyphenyl)-1H-pyrrolo[2,3-b]pyridin-6-yl]-2-(piperazin-1-ylmethyl)cyclopropane-1-carboxamide Enantiomer 2: LCMS (ESI, m/z): [M+H]⁺=436.3. ¹H NMR (400 MHz, DMSO-d₆) δ 11.40 (s, 1H), 10.51 (s, 1H), 7.81 (d, J=8.8 Hz, 1H), 7.52 (d, J=8.8 Hz, 1H), 7.29-7.25 (m, 2H), 6.74 (d, J=8.4 Hz, 2H), 3.68 (s, 6H), 2.73-2.67 (m, 4H), 2.38-2.32 (m, 5H), 2.22-2.18 (m, 1H), 1.89-1.87 (m, 1H), 1.35-1.31 (m, 1H), 1.04-0.98 (m, 1H), 0.78-0.61 (m, 1H).

### Example S151. Compound 153

### Step 1: Synthesis of 5-(6-chloro-1-[[2-(trimethylsilyl)ethoxy]methyl]pyrrolo[2,3-b]pyridin-3-yl)-4-cyclopropoxy-6-methoxypyrimidine

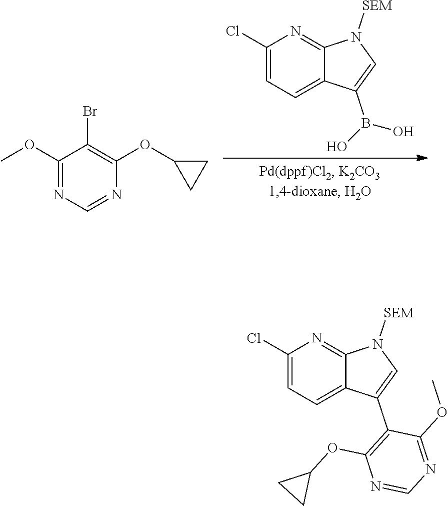 [SMILES: COc1ncnc(OC2CC2)c1Br ; C[Si](C)(C)CCOCn1cc(B(O)O)c2ccc(Cl)nc21 ; COc1ncnc(OC2CC2)c1-c1cn(COCC[Si](C)(C)C)c2nc(Cl)ccc12]

To a solution of 5-bromo-4-cyclopropoxy-6-methoxypyrimidine (500.0 mg, 2.04 mmol) in dioxane/H₂O (20.0 mL/4.0 mL) was added 6-chloro-1-[[2-(trimethylsilyl)ethoxy]methyl]pyrrolo[2,3-b]pyridin-3-ylboronic acid (666.5 mg, 2.04 mmol), K₂CO₃ (845.9 mg, 6.12 mmol) and Pd(dppf)Cl₂ (149.3 mg, 0.20 mmol) at room temperature under N₂. The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 5-(6-chloro-1-[[2-(trimethylsilyl)ethoxy]methyl]pyrrolo[2,3-b]pyridin-3-yl)-4-cyclopropoxy-6-methoxypyrimidine (260.0 mg, 29%) as a yellow oil. LCMS (ESI, m/z): [M+H]⁺=447.2.

### Step 2: Synthesis of (1S,2S)—N-[3-(4-cyclopropoxy-6-methoxypyrimidin-5-yl)-1-[[2-(trimethylsilyl)ethoxy]methyl]pyrrolo[2,3-b]pyridin-6-yl]-2-fluorocyclopropane-1-carboxamide

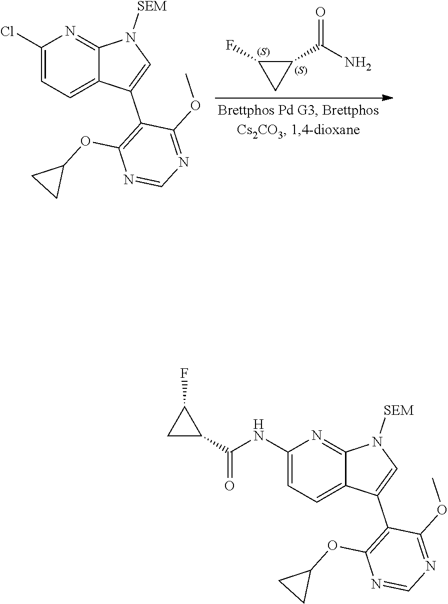

To a solution of 5-(6-chloro-1-[[2-(trimethylsilyl)ethoxy]methyl]pyrrolo[2,3-b]pyridin-3-yl)-4-cyclopropoxy-6-methoxypyrimidine (240.0 mg, 0.54 mmol) in 1,4-dioxane (10.0 mL) was added (1S,2S)-2-fluorocyclopropane-1-carboxamide (276.8 mg, 2.68 mmol), BrettPhos (57.6 mg, 0.11 mmol), Cs₂CO₃ (524.8 mg, 1.61 mmol) and BrettPhos Pd G3 (48.67 mg, 0.054 mmol) at room temperature under N₂. The resulting mixture was stirred at 100° C. for 4 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-[3-(4-cyclopropoxy-6-methoxypyrimidin-5-yl)-1-[[2-(trimethylsilyl)ethoxy]methyl]pyrrolo[2,3-b]pyridin-6-yl]-2-fluorocyclopropane-1-carboxamide (150.0 mg, 54%) as a yellow oil. LCMS (ESI, m/z): [M+H]⁺=514.2.

### Step 3: Synthesis of (1S,2S)—N-[3-(4-cyclopropoxy-6-methoxypyrimidin-5-yl)-1H-pyrrolo[2,3-b]pyridin-6-yl]-2-fluorocyclopropane-1-carboxamide (Compound 153)

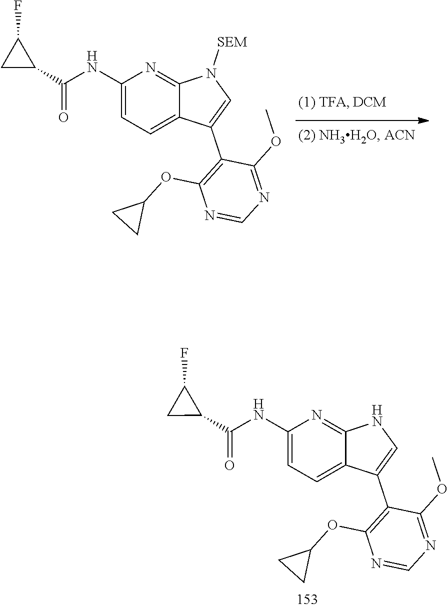 [SMILES: COc1ncnc(OC2CC2)c1-c1cn(COCC[Si](C)(C)C)c2nc(NC(=O)[C@@H]3C[C@@H]3F)ccc12 ; COc1ncnc(OC2CC2)c1-c1c[nH]c2nc(NC(=O)[C@@H]3C[C@@H]3F)ccc12]

To a solution of (1S,2S)—N-[3-(4-cyclopropoxy-6-methoxypyrimidin-5-yl)-1-[[2-(trimethylsilyl)ethoxy]methyl]pyrrolo[2,3-b]pyridin-6-yl]-2-fluorocyclopropane-1-carboxamide (150.0 mg, 0.12 mmol) in CH₂Cl₂ (10.0 mL) was added TFA (10.0 mL) at room temperature. The resulting mixture was stirred at room temperature for 4 h. The reaction mixture was evaporated in vacuo. To the residue in CH₃CN (10.0 mL) was added NH₃·H₂O (10.0 mL) at room temperature. The resulting mixture was stirred at room temperature for another 16 h. After the reaction was completed, the resulting mixture was concentrated under reduced pressure. The residue was purified by Prep-HPLC with the following conditions Column: (Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 41% B in 8 min; 254 nm) to afford (1S,2S)—N-[3-(4-cyclopropoxy-6-methoxypyrimidin-5-yl)-1H-pyrrolo[2,3-b]pyridin-6-yl]-2-fluorocyclopropane-1-carboxamide (17.2 mg, 20%) as a white solid. LCMS (ESI, m/z): [M+H]⁺=384.2. ¹H NMR (400 MHz, DMSO-d₆): δ 11.65 (s, 1H), 10.63 (s, 1H), 8.49 (s, 1H), 7.86 (d, J=8.4 Hz, 1H), 7.67 (d, J=8.4 Hz, 1H), 7.45 (d, J=2.8 Hz, 1H), 5.01-4.83 (m, 1H), 4.38-4.34 (m, 1H), 3.92 (s, 3H), 2.28-2.22 (m, 1H), 1.68-1.61 (m, 1H), 1.18-1.14 (m, 1H), 0.82-0.71 (m, 2H), 0.69-0.61 (m, 2H).

### Example S152. Compound 154

### Step 1: Synthesis of 4,6-dimethoxypyridin-2-amine

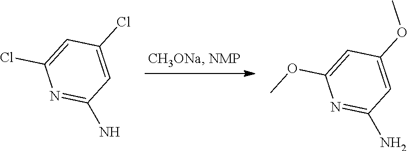

To a solution of 4,6-dichloropyridin-2-amine (5.0 g, 30.68 mmol) in NMP (30.0 mL) was added CH₃ONa (8.3 g, 153.38 mmol). The resulting mixture was stirred at 120° C. for 16 h. After the reaction was completed, the reaction was diluted with water and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (70/30, v/v) to afford 4,6-dimethoxypyridin-2-amine (2.7 g, 57%) as a yellow solid. LCMS (ESI, m/z): [M+H]⁺=155.1.

### Step 2: Synthesis of 3-benzoyl-1-(4,6-dimethoxypyridin-2-yl)thiourea

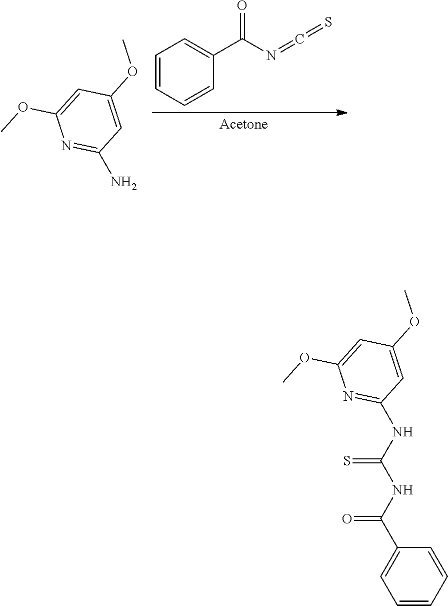

To a solution of 4,6-dimethoxypyridin-2-amine (1.0 g, 6.49 mmol) in acetone (10.0 mL) was added benzoyl isothiocyanate (1.2 g, 7.14 mmol). The mixture was stirred at room temperature for 1 h. After the reaction was completed, the reaction mixture was filtered. The solid was collected and dried to afford 3-benzoyl-1-(4,6-dimethoxypyridin-2-yl)thiourea (1.8 g, crude) as a yellow solid. LCMS (ESI, m/z): [M+H]⁺=318.1.

### Step 3: Synthesis of 4,6-dimethoxypyridin-2-ylthiourea

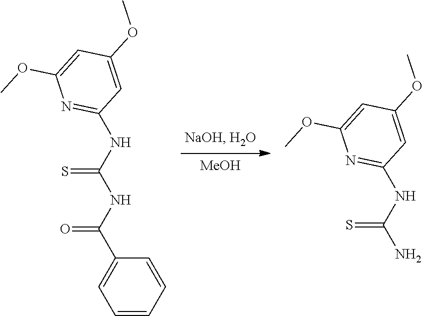 [SMILES: "MeOH" ; COc1cc(NC(=S)NC(=O)c2ccccc2)nc(OC)c1 ; COc1cc(NC(N)=S)nc(OC)c1]

To a solution of 3-benzoyl-1-(4,6-dimethoxypyridin-2-yl)thiourea (1.7 g, 5.36 mmol) in MeOH/H₂O (15.0 mL/3.0 mL) was added NaOH (428.5 mg, 10.71 mmol). The mixture was stirred at room temperature for 2 h. After the reaction was completed, the mixture was filtered. The solid was washed with H₂O and dried to afford 4,6-dimethoxypyridin-2-ylthiourea (1.1 g, crude) as a light yellow solid. LCMS (ESI, m/z): [M+H]⁺=214.1.

### Step 4: Synthesis of 6-bromo-5,7-dimethoxy-[1,3]thiazolo[4,5-b]pyridin-2-amine

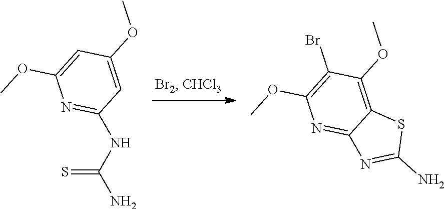

To a solution of 4,6-dimethoxypyridin-2-ylthiourea (1.0 g, 4.69 mmol) in CHCl₃ (10.0 mL) was added dropwise Br₂ (1.5 g, 9.38 mmol) at 0° C. The mixture was stirred at 60° C. for 1 h. After the reaction was completed, the reaction mixture was concentrated under vacuum. The residue was purified by flash column chromatography with CH₂Cl₂/MeOH (90/10, v/v) to afford 6-bromo-5,7-dimethoxy-[1,3]thiazolo[4,5-b]pyridin-2-amine (1.2 g, 88%) as a yellow solid. LCMS (ESI, m/z): [M+H]⁺=290.0.

### Step 5: Synthesis of 6-bromo-5,7-dimethoxy-[1,3]thiazolo[4,5-b]pyridine

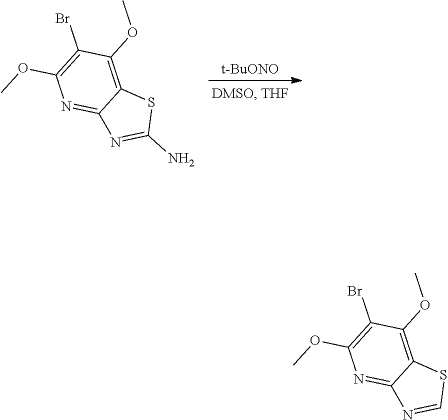

To a solution of 6-bromo-5,7-dimethoxy-[1,3]thiazolo[4,5-b]pyridin-2-amine (1.1 g, 3.79 mmol) in THF (10.0 mL) was added 2-methyl-2-propylnitrite (469.2 mg, 4.55 mmol) and DMSO (50.0 uL). The mixture was stirred at room temperature for 2 h. After the reaction was completed, the reaction mixture was concentrated under vacuum. The residue was purified by flash column chromatography with CH₂Cl₂/MeOH (90/10, v/v) to afford 6-bromo-5,7-dimethoxy-[1,3]thiazolo[4,5-b]pyridine (320.0 mg, 30%) as a yellow solid. LCMS (ESI, m/z): [M+H]⁺=274.9.

### Step 6: Synthesis of 6-(6-chloro-1-{[2-(trimethylsilyl)ethoxy]methyl}pyrrolo[2,3-b]pyridin-3-yl)-5,7-dimethoxy-[1,3]thiazolo[4,5-b]pyridine

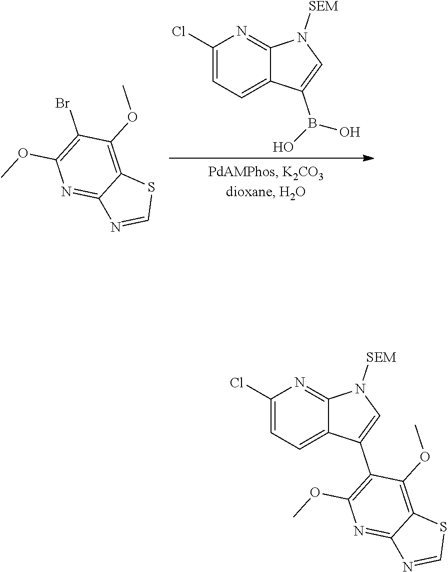 [SMILES: COc1nc2ncsc2c(OC)c1Br ; C[Si](C)(C)CCOCn1cc(B(O)O)c2ccc(Cl)nc21 ; COc1nc2ncsc2c(OC)c1-c1cn(COCC[Si](C)(C)C)c2nc(Cl)ccc12]

To a solution of 6-bromo-5,7-dimethoxy-[1,3]thiazolo[4,5-b]pyridine (260.0 mg, 0.95 mmol) in 1,4-dioxane/H₂O (5.0 mL/1.0 mL) was added 6-chloro-1-{[2-(trimethylsilyl)ethoxy]methyl}pyrrolo[2,3-b]pyridin-3-ylboronic acid (308.7 mg, 0.95 mmol), K₃PO₄ (601.8 mg, 2.84 mmol) and PdAMPHOS (66.9 mg, 0.10 mmol). The mixture was stirred at 80° C. for 2 h under N₂. After the reaction was completed, the reaction mixture was concentrated under vacuum. The residue was purified by flash column chromatography with petroleum ether/ethyl acetate (75/25, v/v) to afford 6-(6-chloro-1-{[2-(trimethylsilyl)ethoxy]methyl}pyrrolo[2,3-b]pyridin-3-yl)-5,7-dimethoxy-[1,3]thiazolo[4,5-b]pyridine (220.0 mg, 48%) as a yellow solid. LCMS (ESI, m/z): [M+H]⁺=477.1.

### Step 7: Synthesis of (1S,2S)—N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-fluorocyclopropane-1-carboxamide

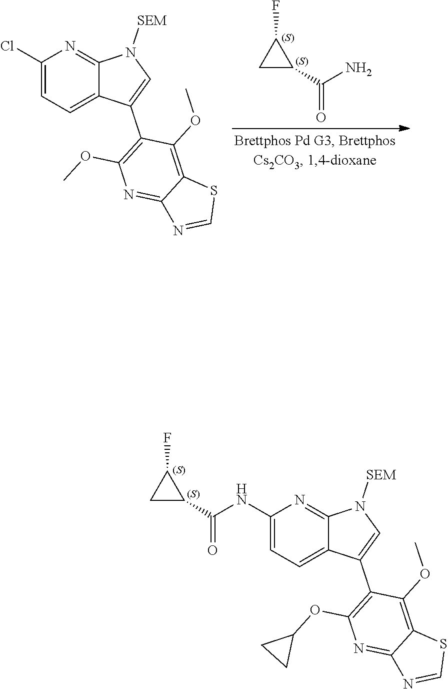

To a solution of 6-(6-chloro-1-{[2-(trimethylsilyl)ethoxy]methyl}pyrrolo[2,3-b]pyridin-3-yl)-5,7-dimethoxy-[1,3]thiazolo[4,5-b]pyridine (200.0 mg, 0.42 mmol) in 1,4-dioxane (5.0 mL) was added (1S,2S)-2-fluorocyclopropane-1-carboxamide (216.1 mg, 2.10 mmol), Cs₂CO₃ (409.7 mg, 1.257 mmol), BrettPhos (45.0 mg, 0.08 mmol) and BrettPhos Pd G3 (38.0 mg, 0.04 mmol) under N₂. The mixture was stirred with microwave at 120° C. for 1 h. After the reaction was completed, the reaction was concentrated under vacuum. The residue was purified by flash column chromatography with CH₂Cl₂/MeOH (90/10, v/v) to afford (1S,2S)—N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-fluorocyclopropane-1-carboxamide (160.0 mg, 70%) as a yellow solid. LCMS (ESI, m/z): [M+H]⁺=544.2.

### Step 8: Synthesis of (1S,2S)—N-(3-{5,7-dimethoxy-[1,3]thiazolo[4,5-b]pyridin-6-yl}-1H-pyrrolo[2,3-b]pyridin-6-yl)-2-fluorocyclopropane-1-carboxamide (Compound 154)

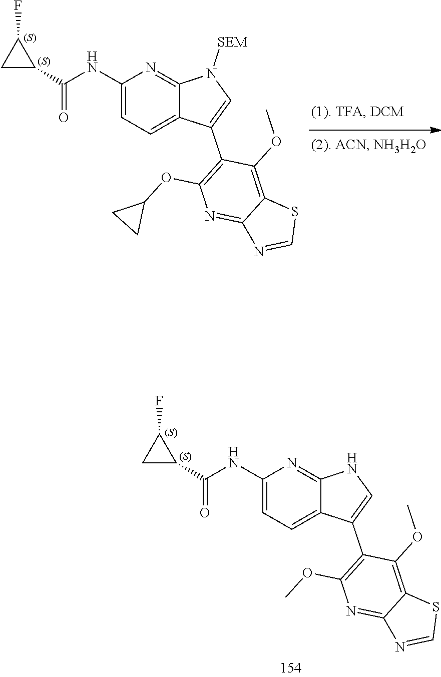

To a solution of (1S,2S)—N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-fluorocyclopropane-1-carboxamide (140.0 mg, 0.26 mmol) in CH₂Cl₂ (3.0 mL) was added TFA (3.0 mL). The mixture was stirred at room temperature for 3 h. The mixture was concentrated under vacuum. To the above mixture were added ACN (3.0 mL) and NH₃·H₂O (3.0 mL). The resulting mixture was stirred at room temperature for another 3 h. After the reaction was completed, the resulting mixture was concentrated under vacuum. The residue was purified by Prep-HPLC with the following conditions (Column: XBridge Prep OBD Column, 30×150 mm, 5 um; Mobile Phase A: Water (10 mmol/L NH₄HCO₃), Mobile Phase B: ACN; Flow rate: 60 mL/min; Gradient: 26% B to 35% B in 9 min; 254 nm) to afford (1S,2S)—N-(3-{5,7-dimethoxy-[1,3]thiazolo[4,5-b]pyridin-6-yl}-1H-pyrrolo[2,3-b]pyridin-6-yl)-2-fluorocyclopropane-1-carboxamide (31.0 mg, 29%) as a white solid. LCMS (ESI, m/z): [M+H]⁺=414.1. ¹H NMR (300 MHz, DMSO-d₆): δ 11.68 (s, 1H), 10.65 (s, 1H), 9.56 (s, 1H), 7.86 (d, J=8.4 Hz, 1H), 7.71 (d, J=8.7 Hz, 1H), 7.48 (d, J=2.4 Hz, 1H), 5.06-4.78 (m, 1H), 3.90 (s, 6H), 2.28-2.23 (m, 1H), 1.72-1.58 (m, 1H), 1.18-1.05 (m, 1H).

### Example S153. Compound 155 and Compound 156

### Step 1: Synthesis of trans-methyl 2-[[3-(2,6-dimethoxyphenyl)-1-[[2-(trimethylsilyl)ethoxy]methyl]pyrrolo[2,3-b]pyridin-6-yl]carbamoyl]cyclopropane-1-carboxylate

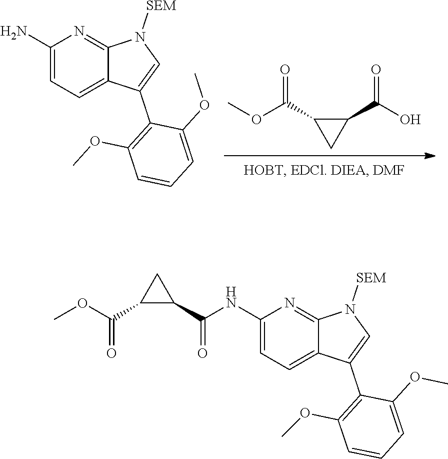

To a solution of trans-2-(methoxycarbonyl)cyclopropane-1-carboxylic acid (144.3 mg, 1.00 mmol) in DMF (20.0 mL) were added EDCI (287.9 mg, 1.50 mmol) and HOBT (202.9 mg, 1.50 mmol) at room temperature. The resulting mixture was stirred at room temperature for 20 min. Then DIEA (517.5 mg, 4.00 mmol) and 3-(2,6-dimethoxyphenyl)-1-[[2-(trimethylsilyl)ethoxy]methyl]pyrrolo[2,3-b]pyridin-6-amine (400.0 mg, 1.00 mmol) were added to the mixture at room temperature. The resulting mixture was stirred at room temperature for 16 h. After the reaction was completed, the mixture was purified by reverse phase flash chromatography with H₂O/CH₃CN (1/1, v/v) to afford trans-methyl 2-[[3-(2,6-dimethoxyphenyl)-1-[[2-(trimethylsilyl)ethoxy]methyl]pyrrolo[2,3-b]pyridin-6-yl]carbamoyl]cyclopropane-1-carboxylate (280.0 mg, 53%) as a white solid. LCMS (ESI, m/z): [M+H]⁺=526.2.

### Step 2: Synthesis of trans-N-[3-(2,6-dimethoxyphenyl)-1-[[2-(trimethylsilyl)ethoxy]methyl]pyrrolo[2,3-b]pyridin-6-yl]-2-(hydroxymethyl)cyclopropane-1-carboxamide

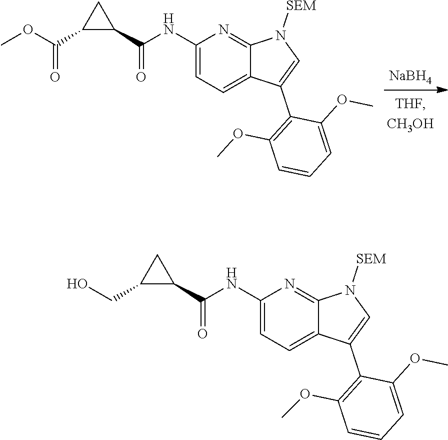

To a solution of trans-methyl 2-[[3-(2,6-dimethoxyphenyl)-1-[[2-(trimethylsilyl)ethoxy]methyl]pyrrolo[2,3-b]pyridin-6-yl]carbamoyl]cyclopropane-1-carboxylate (330.0 mg, 0.63 mmol) in THF (33.0 mL)/CH₃OH (33.0 mL) was added NaBH₄ (712.5 mg, 18.83 mmol) at room temperature. The resulting mixture was stirred at room temperature for 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure to afford trans-N-[3-(2,6-dimethoxyphenyl)-1-[[2-(trimethylsilyl)ethoxy]methyl]pyrrolo[2,3-b]pyridin-6-yl]-2-(hydroxymethyl)cyclopropane-1-carboxamide (300.0 mg, 96%) as a yellow oil. LCMS (ESI, m/z): [M+H]⁺=498.2.

### Step 3: Synthesis of trans-N-[3-(2,6-dimethoxyphenyl)-1-[[2-(trimethylsilyl)ethoxy]methyl]pyrrolo[2,3-b]pyridin-6-yl]-2-formylcyclopropane-1-carboxamide

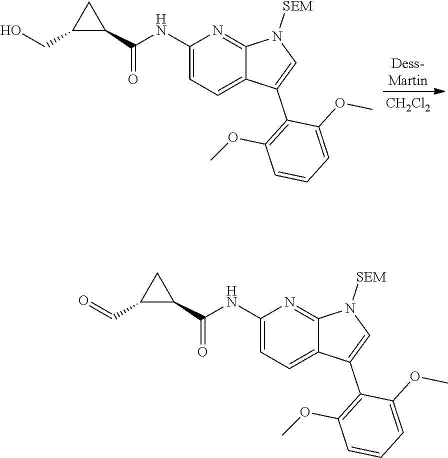

To a solution of trans-N-[3-(2,6-dimethoxyphenyl)-1-[[2-(trimethylsilyl)ethoxy]methyl]pyrrolo[2,3-b]pyridin-6-yl]-2-(hydroxymethyl)cyclopropane-1-carboxamide (300.0 mg, 0.60 mmol) in CH₂Cl₂ (30.0 mL) was added Dess-Martin (767.0 mg, 1.81 mmol) at room temperature. The resulting mixture was stirred at room temperature for 1 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure to afford trans-N-[3-(2,6-dimethoxyphenyl)-1-[[2-(trimethylsilyl)ethoxy]methyl]pyrrolo[2,3-b]pyridin-6-yl]-2-formylcyclopropane-1-carboxamide (260.0 mg, crude) as a brown solid. LCMS (ESI, m/z): [M+H]⁺=496.2.

### Step 4: Synthesis of trans-N-(3-(2,6-dimethoxyphenyl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide

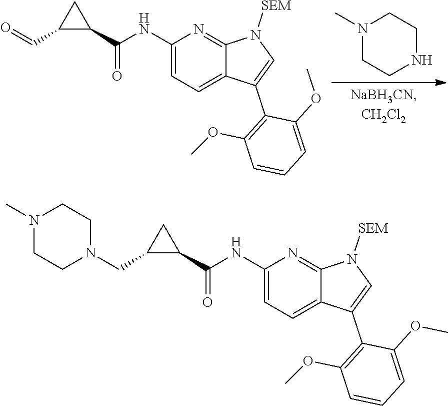

To a solution of trans-N-[3-(2,6-dimethoxyphenyl)-1-[[2-(trimethylsilyl)ethoxy]methyl]pyrrolo[2,3-b]pyridin-6-yl]-2-formylcyclopropane-1-carboxamide (350.0 mg, crude) in CH₂Cl₂ (20.0 mL) was added 1-methylpiperazine (212.2 mg, 2.12 mmol) and NaBH₃CN (133.1 mg, 2.12 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with CH₂Cl₂/CH₃OH (86/14, v/v) to afford trans-N-(3-(2,6-dimethoxyphenyl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide (180.0 mg, 43%) as a yellow solid. LCMS (ESI, m/z): [M+H]⁺=580.3.

### Step 5: Synthesis of (1R,2R)—N-(3-(2,6-dimethoxyphenyl)-1H-pyrrolo[2,3-b]pyridin-6-yl)-2-((4-methylpiperazin-1-yl)methyl)cyclopropane-1-carboxamide and (1S,2S)—N-(3-(2,6-dimethoxyphenyl)-1H-pyrrolo[2,3-b]pyridin-6-yl)-2-((4-methylpiperazin-1-yl)methyl)cyclopropane-1-carboxamide (Compound 155 and Compound 156)

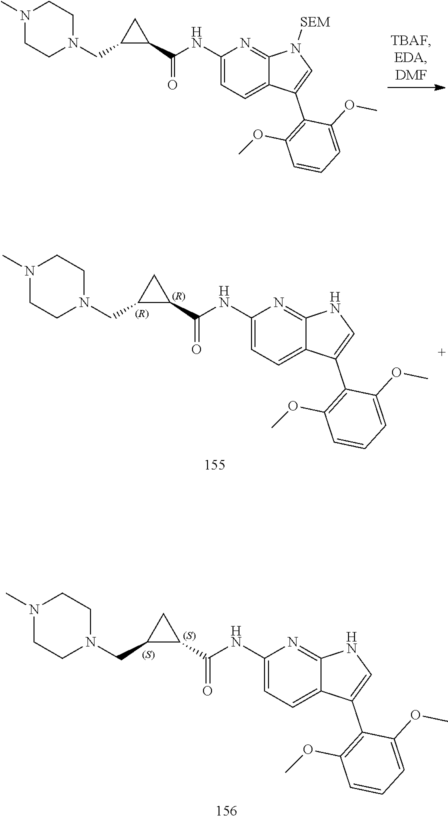

To a solution of trans-N-(3-(2,6-dimethoxyphenyl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide (180.0 mg, 0.31 mmol) in DMF (5.0 mL) was added ethylenediamine (93.3 mg, 1.55 mmol) and TBAF (243.5 mg, 0.93 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (75/25, v/v) and then separated by Prep-Chiral-HPLC with the following conditions (Column: CHIRALPAK IE, 2×25 cm, 5 um; Mobile Phase A: Hex (0.5% 2M NH₃-MeOH)—HPLC, Mobile Phase B: EtOH—HPLC; Flow rate: 20 mL/min; Gradient: 10% to 10% in 46 min; 220/254 nm) to afford N-(3-(2,6-dimethoxyphenyl)-1H-pyrrolo[2,3-b]pyridin-6-yl)-2-((4-methylpiperazin-1-yl)methyl)cyclopropane-1-carboxamide Enantiomer 1 (10.3 mg, 7%, Retention time 1: 16.876 min) as a white solid and N-(3-(2,6-dimethoxyphenyl)-1H-pyrrolo[2,3-b]pyridin-6-yl)-2-((4-methylpiperazin-1-yl)methyl)cyclopropane-1-carboxamide Enantiomer 1 (12.3 mg, 8%, Retention time 2: 30.093 min) as a white solid. The absolute stereochemistry of Enantiomers 1 and 2 was not assigned. The two enantiomeric structures that could be obtained from chiral separation of the enantiomeric mixture as described above are shown as Compounds 155 and 156 in Table 1.

N-(3-(2,6-dimethoxyphenyl)-1H-pyrrolo[2,3-b]pyridin-6-yl)-2-((4-methylpiperazin-1-yl)methyl)cyclopropane-1-carboxamide Enantiomer 1: LCMS (ESI, m/z): [M+H]⁺=450.3. ¹H NMR (400 MHz, DMSO-d₆): δ 11.42 (s, 1H), 10.53 (s, 1H), 7.82 (d, J=8.4 Hz, 1H), 7.53 (d, J=8.8 Hz, 1H), 7.29-7.25 (m, 2H), 6.75 (d, J=8.4 Hz, 2H), 3.69 (s, 6H), 2.49-2.26 (m, 8H), 2.14 (s, 3H), 1.97-1.89 (m, 1H), 1.33-1.23 (m, 1H), 1.04-1.02 (m, 1H), 0.73-0.65 (m, 1H).

N-(3-(2,6-dimethoxyphenyl)-1H-pyrrolo[2,3-b]pyridin-6-yl)-2-((4-methylpiperazin-1-yl)methyl)cyclopropane-1-carboxamide Enantiomer 2: LCMS (ESI, m/z): [M+H]⁺=450.2. ¹H NMR (400 MHz, DMSO-d₆): δ 11.42 (s, 1H), 10.53 (s, 1H), 7.82 (d, J=7.6 Hz, 1H), 7.53 (d, J=8.0 Hz, 1H), 7.29-7.25 (m, 2H), 6.75 (d, J=8.0 Hz, 2H), 3.69 (s, 6H), 2.49-2.24 (m, 8H), 2.14 (s, 3H), 1.97-1.89 (m, 1H), 1.33-1.23 (m, 1H), 1.04-1.02 (m, 1H), 0.73-0.65 (m, 1H).

### Example S154. Compound 157 and Compound 158

### Step 1: Synthesis of trans-N-[3-(2,6-dimethoxyphenyl)-1-{[2-(trimethylsilyl)ethoxy]methyl}pyrrolo[2,3-b]pyridin-6-yl]-2-formylcyclopropane-1-carboxamide

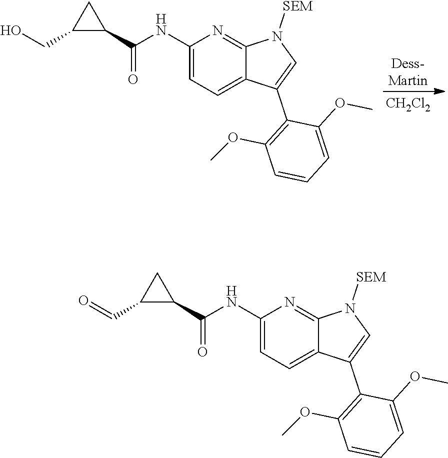

To a solution of trans-N-[3-(2,6-dimethoxyphenyl)-1-{[2-(trimethylsilyl)ethoxy]methyl}pyrrolo[2,3-b]pyridin-6-yl]-2-(hydroxymethyl)cyclopropane-1-carboxamide (400.0 mg, 0.80 mmol) in CH₂Cl₂ (5.0 mL) was added Dess-Martin (1022.7 mg, 2.41 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure to afford trans-N-[3-(2,6-dimethoxyphenyl)-1-{[2-(trimethylsilyl)ethoxy]methyl}pyrrolo[2,3-b]pyridin-6-yl]-2-formylcyclopropane-1-carboxamide (500.0 mg, crude) as a brown solid. LCMS (ESI, m/z): [M+H]⁺=496.2.

### Step 2: Synthesis of trans-N-[3-(2,6-dimethoxyphenyl)-1-{[2-(trimethylsilyl)ethoxy]methyl}pyrrolo[2,3-b]pyridin-6-yl]-2-(morpholin-4-ylmethyl)cyclopropane-1-carboxamide

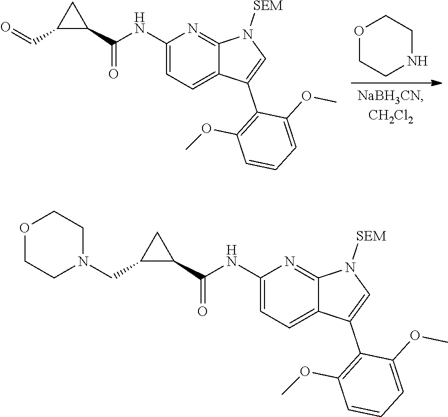

To a solution of trans-N-[3-(2,6-dimethoxyphenyl)-1-{[2-(trimethylsilyl)ethoxy]methyl}pyrrolo[2,3-b]pyridin-6-yl]-2-formylcyclopropane-1-carboxamide (500.0 mg, crude) in CH₂Cl₂ (10.0 mL) was added morpholine (262.6 mg, 3.03 mmol) and NaBH₃CN (189.4 mg, 3.03 mmol) at room temperature. The resulting mixture was stirred at room temperature for 2 h. After the reaction was completed, the resulting mixture was quenched with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum CH₂Cl₂/MeOH (95/5, v/v) to afford trans-N-[3-(2,6-dimethoxyphenyl)-1-{[2-(trimethylsilyl)ethoxy]methyl}pyrrolo[2,3-b]pyridin-6-yl]-2-(morpholin-4-ylmethyl)cyclopropane-1-carboxamide (410.0 mg, 59%) as a brown solid. LCMS (ESI, m/z): [M+H]⁺=567.3.

### Step 3: Synthesis of trans-N-(3-(2,6-dimethoxyphenyl)-1H-pyrrolo[2,3-b]pyridin-6-yl)-2-(morpholinomethyl)cyclopropanecarboxamide

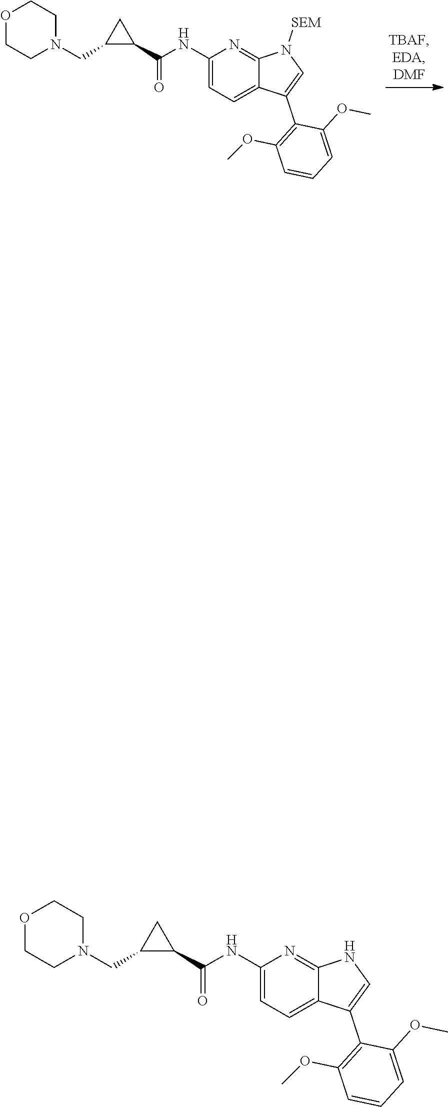 [SMILES: COc1cccc(OC)c1-c1cn(COCC[Si](C)(C)C)c2nc(NC(=O)[C@@H]3C[C@H]3CN3CCOCC3)ccc12 ; COc1cccc(OC)c1-c1c[nH]c2nc(NC(=O)[C@@H]3C[C@H]3CN3CCOCC3)ccc12]

To a solution of trans-N-[3-(2,6-dimethoxyphenyl)-1-{[2-(trimethylsilyl)ethoxy]methyl}pyrrolo[2,3-b]pyridin-6-yl]-2-(morpholin-4-ylmethyl)cyclopropane-1-carboxamide (390.0 mg, 0.69 mmol) in DMF (5.0 mL) was added TBAF (539.7 mg, 2.06 mmol) and DEA (206.8 mg, 3.44 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum CH₂Cl₂/MeOH (90/10, v/v) and then purified by reverse phase flash chromatography with ACN/H₂O (50/50, v/v) to afford trans-N-(3-(2,6-dimethoxyphenyl)-1H-pyrrolo[2,3-b]pyridin-6-yl)-2-(morpholinomethyl)cyclopropanecarboxamide (100.0 mg, 30%) as a white solid. LCMS (ESI, m/z): [M+H]⁺=437.2.

### Step 4: Synthesis of (1R,2R)—N-(3-(2,6-dimethoxyphenyl)-1H-pyrrolo[2,3-b]pyridin-6-yl)-2-(morpholinomethyl)cyclopropanecarboxamide and (1S,2S)—N-(3-(2,6-dimethoxyphenyl)-1H-pyrrolo[2,3-b]pyridin-6-yl)-2-(morpholinomethyl)cyclopropanecarboxamide (Compound 157 and Compound 158)

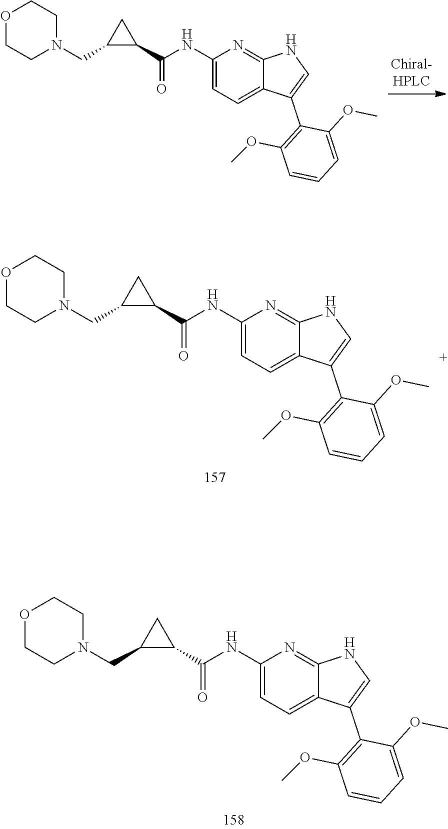 [SMILES: COc1cccc(OC)c1-c1c[nH]c2nc(NC(=O)[C@@H]3C[C@H]3CN3CCOCC3)ccc12 ; COc1cccc(OC)c1-c1c[nH]c2nc(NC(=O)[C@@H]3C[C@H]3CN3CCOCC3)ccc12 ; COc1cccc(OC)c1-c1c[nH]c2nc(NC(=O)[C@H]3C[C@@H]3CN3CCOCC3)ccc12]

The racemic trans-N-(3-(2,6-dimethoxyphenyl)-1H-pyrrolo[2,3-b]pyridin-6-yl)-2-(morpholinomethyl)cyclopropanecarboxamide (100.0 mg, 0.23 mmol) was separated by Prep-Chiral-HPLC with the following conditions (Column: CHIRALPAK IE, 2×25 cm, 5 um; Mobile Phase A: Hex (0.5% 2M NH₃-MeOH)—HPLC, Mobile Phase B: EtOH—HPLC; Flow rate: 18 mL/min; Gradient: 50% B to 50% B in 23 min; 254/220 nm) to afford N-[3-(2,6-dimethoxyphenyl)-1H-pyrrolo[2,3-b]pyridin-6-yl]-2-(morpholin-4-ylmethyl)cyclopropane-1-carboxamide Enantiomer 1 (22.2 mg, 44%, Retention time 1: 11.1 min) as a white solid and (1S,2S)—N-[3-(2,6-dimethoxyphenyl)-1H-pyrrolo[2,3-b]pyridin-6-yl]-2-(morpholin-4-ylmethyl)cyclopropane-1-carboxamide Enantiomer 2 (assumed, 28.4 mg, 56%, Retention time 2: 15.904 min) as a white solid. The absolute stereochemistry of Enantiomers 1 and 2 was not assigned. The two enantiomeric structures that could be obtained from chiral separation of the enantiomeric mixture as described above are shown as Compounds 157 and 158 in Table 1.

N-[3-(2,6-dimethoxyphenyl)-1H-pyrrolo[2,3-b]pyridin-6-yl]-2-(morpholin-4-ylmethyl)cyclopropane-1-carboxamide Enantiomer 1: LCMS (ESI, m/z): [M+H]⁺=437.3. ¹H NMR (400 MHz, DMSO-d₆): δ 11.42 (s, 1H), 10.54 (s, 1H), 7.81 (d, J=8.4 Hz, 1H), 7.52 (d, J=8.4 Hz, 1H), 7.30-7.25 (m, 2H), 6.75 (d, J=8.4 Hz, 2H), 3.69 (s, 6H), 3.59-3.57 (m, 4H), 2.50-2.43 (m, 4H), 2.38-2.33 (m, 1H), 2.27-2.25 (m, 1H), 1.95-1.86 (m, 1H), 1.41-1.32 (m, 1H), 1.11-0.99 (m, 1H), 0.77-0.65 (m, 1H).

N-[3-(2,6-dimethoxyphenyl)-1H-pyrrolo[2,3-b]pyridin-6-yl]-2-(morpholin-4-ylmethyl)cyclopropane-1-carboxamide Enantiomer 2: LCMS (ESI, m/z): [M+H]⁺=437.3. ¹H NMR (400 MHz, DMSO-d₆) δ 11.42 (s, 1H), 10.54 (s, 1H), 7.81 (d, J=8.8 Hz, 1H), 7.52 (d, J=8.8 Hz, 1H), 7.30-7.25 (m, 2H), 6.75 (d, J=8.4 Hz, 2H), 3.69 (s, 6H), 3.59-3.57 (m, 4H), 2.50-2.43 (m, 4H), 2.38-2.33 (m, 1H), 2.27-2.25 (m, 1H), 1.95-1.86 (m, 1H), 1.41-1.32 (m, 1H), 1.11-0.99 (m, 1H), 0.77-0.65 (m, 1H).

### Example S155: Synthesis of (1S,2S)-2-fluoro-N-(3-[7-methoxy-[1,2,4]triazolo[4,3-a]pyridin-6-yl]-1H-pyrrolo[2,3-b]pyridin-6-yl)cyclopropane-1-carboxamide (Compound 159)

### Step 1: Synthesis of 5-bromo-2-hydrazinyl-4-methoxypyridine

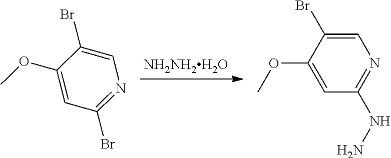

The solution of 2,5-dibromo-4-methoxypyridine (500.0 mg, 2.24 mmol) in NH₂NH₂·H₂O (50.0 mL, 80%) was stirred at 80° C. for 16 h. After the reaction was completed, the resulting mixture was concentrated under reduced pressure. The residue was purified by reverse phase flash chromatography with CH₃CN/H₂O (1/4, v/v) to afford 5-bromo-2-hydrazinyl-4-methoxypyridine (60.0 mg, 12%) as a yellow solid. LCMS (ESI, m/z): [M+H]⁺=218.0

### Step 2: Synthesis of 6-bromo-7-methoxy-[1,2,4]triazolo[4,3-a]pyridine

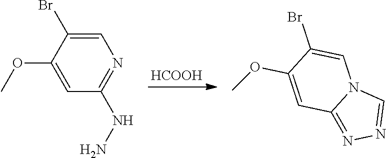

The solution of 5-bromo-2-hydrazinyl-4-methoxypyridine (600.0 mg, 2.75 mmol) in formic acid (10.0 mL) was stirred at 100° C. for 16 h. After the reaction was completed, the resulting mixture was concentrated under reduced pressure. The residue was purified by reverse phase flash chromatography with MeOH/H₂O (1/10, v/v) to afford 6-bromo-7-methoxy-[1,2,4]triazolo[4,3-a]pyridine (120.0 mg, 19%) as a yellow solid. LCMS (ESI, m/z): [M+H]⁺=228.0

### Step 3: Synthesis of 6-chloro-3-[7-methoxy-[1,2,4]triazolo[4,3-a]pyridin-6-yl]-1-[[2-(trimethylsilyl)ethoxy]methyl]pyrrolo[2,3-b]pyridine

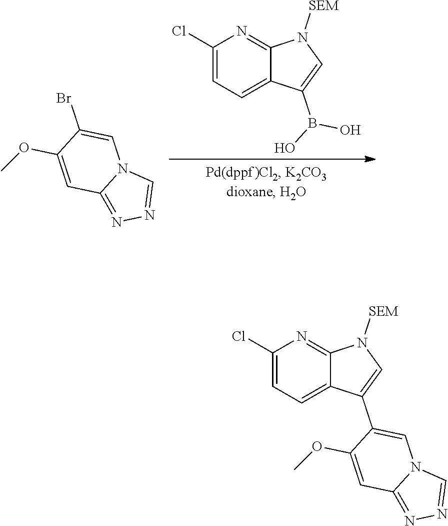 [SMILES: COc1cc2nncn2cc1Br ; C[Si](C)(C)CCOCn1cc(B(O)O)c2ccc(Cl)nc21 ; COc1cc2nncn2cc1-c1cn(COCC[Si](C)(C)C)c2nc(Cl)ccc12]

To a mixture of 6-bromo-7-methoxy-[1,2,4]triazolo[4,3-a]pyridine (100.0 mg, 0.43 mmol) and 6-chloro-1-[[2-(trimethylsilyl)ethoxy]methyl]pyrrolo[2,3-b]pyridin-3-ylboronic acid (214.8 mg, 0.65 mmol) in dioxane/H₂O (5.0/1.0 mL) were added Pd(dppf)Cl₂ (32.0 mg, 0.04 mmol) and K₂CO₃ (181.8 mg, 1.31 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was residue was purified by flash column chromatography with MeOH/DCM (1/10, v/v) to afford 6-chloro-3-[7-methoxy-[1,2,4]triazolo[4,3-a]pyridin-6-yl]-1-[[2-(trimethylsilyl)ethoxy]methyl]pyrrolo[2,3-b]pyridine (110.0 mg, 58%) as a white solid. LCMS (ESI, m/z): [M+H]⁺=430.1

### Step 4: Synthesis of (1S,2S)-2-fluoro-N-(3-[7-methoxy-[1,2,4]triazolo[4,3-a]pyridin-6-yl]-1-[[2-(trimethylsilyl)ethoxy]methyl]pyrrolo[2,3-b]pyridin-6-yl)cyclopropane-1-carboxamide

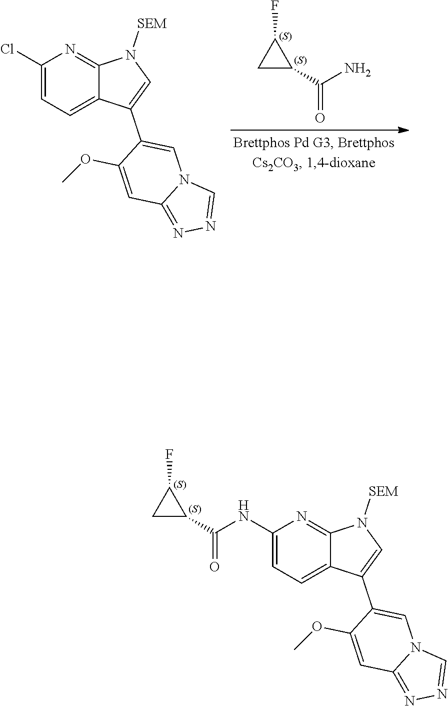

To a mixture of 6-chloro-3-[7-methoxy-[1,2,4]triazolo[4,3-a]pyridin-6-yl]-1-[[2-(trimethylsilyl)ethoxy]methyl]pyrrolo[2,3-b]pyridine (90.0 mg, 0.20 mmol) and (1S,2S)-2-fluorocyclopropane-1-carboxamide (107.9 mg, 1.04 mmol) in dioxane (4.0 mL) was added BrettPhos Pd G3 (37.5 mg, 0.04 mmol), BrettPhos (44.9 mg, 0.08 mmol) and Cs₂CO₃ (204.0 mg, 0.62 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with MeOH/DCM (1/10, v/v) to afford (1S,2S)-2-fluoro-N-(3-[7-methoxy-[1,2,4]triazolo[4,3-a]pyridin-6-yl]-1-[[2-(trimethylsilyl)ethoxy]methyl]pyrrolo[2,3-b]pyridin-6-yl)cyclopropane-1-carboxamide (40.0 mg, 38%) as a yellow solid. LCMS (ESI, m/z): [M+H]⁺=497.2

### Step 5: Synthesis of (1S,2S)-2-fluoro-N-(3-[7-methoxy-[1,2,4]triazolo[4,3-a]pyridin-6-yl]-1H-pyrrolo[2,3-b]pyridin-6-yl)cyclopropane-1-carboxamide (Compound 159)

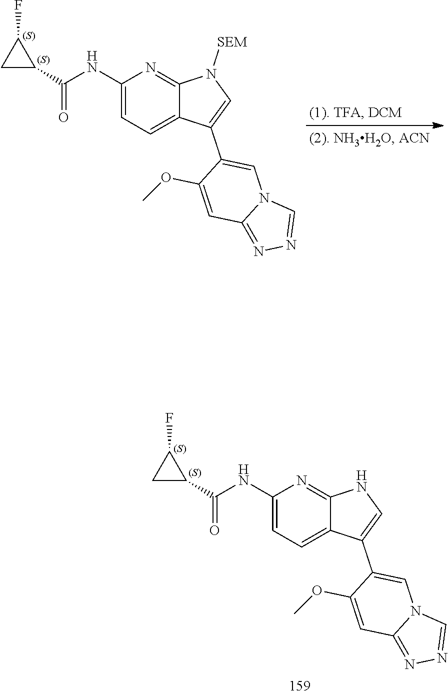

To a solution of (1S,2S)-2-fluoro-N-(3-[7-methoxy-[1,2,4]triazolo[4,3-a]pyridin-6-yl]-1-[[2-(trimethylsilyl)ethoxy]methyl]pyrrolo[2,3-b]pyridin-6-yl)cyclopropane-1-carboxamide (30.0 mg, 0.06 mmol) in DCM (1.0 mL) was added TFA (1.0 mL) at room temperature. The resulting mixture was stirred at room temperature for 2 h. The resulting mixture was concentrated under vacuum. To the above residue was added ACN (2.0 mL) and NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 16% B to 26% B in 8 min, 254 nm) to afford (1S,2S)-2-fluoro-N-(3-[7-methoxy-[1,2,4]triazolo[4,3-a]pyridin-6-yl]-1H-pyrrolo[2,3-b]pyridin-6-yl)cyclopropane-1-carboxamide (Compound 159) (5.0 mg, 22%) as a white solid. LCMS (ESI, m/z): [M+H]⁺=367.1. ¹H NMR (300 MHz, DMSO-d₆): δ 11.77 (s, 1H), 10.68 (s, 1H), 9.06 (s, 1H), 8.68 (s, 1H), 8.12 (d, J=8.7 Hz, 1H), 7.94 (d, J=8.4 Hz, 1H), 7.68 (s, 1H), 7.20 (s, 1H), 5.07-4.80 (m, 1H), 3.95 (s, 3H), 2.27-2.24 (m, 1H), 1.73-1.60 (m, 1H), 1.22-1.10 (m, 1H).

### Example S156: Synthesis of (1S,2S)-2-fluoro-N-(3-[5-methoxy-[1,3]thiazolo[5,4-b]pyridin-6-yl]-1H-pyrrolo[2,3-b]pyridin-6-yl)cyclopropane-1-carboxamide (Compound 160)

### Step 1: Synthesis of 6-bromo-5-methoxy-[1,3]thiazolo[5,4-b]pyridin-2-amine

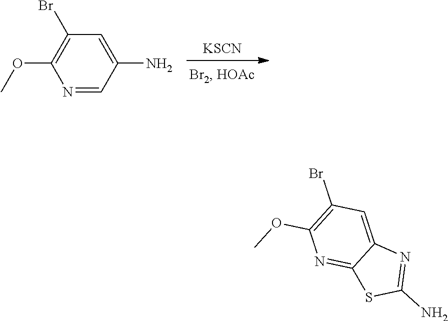

To a mixture of 5-bromo-6-methoxypyridin-3-amine (1.0 g, 4.92 mmol) and KSCN (238.8 mg, 2.46 mmol) in AcOH (20.0 mL) was added Br₂ (390.0 mg, 2.46 mmol) at 0° C. The resulting mixture was stirred at room temperature for 2 h. After the reaction was completed, the pH value of the mixture was adjusted to 9 with aq·NaHCO₃. The resulting mixture was diluted with H₂O and extracted with ethyl acetate. The combined organic layer was washed with brine, dried over anhydrous sodium sulfate and filtered. The filtrate was concentrated under reduced pressure to afford 6-bromo-5-methoxy-[1,3]thiazolo[5,4-b]pyridin-2-amine (2.0 g, crude) as a yellow solid. LCMS (ESI, m/z): [M+H]⁺=259.9

### Step 2: Synthesis of 6-bromo-5-methoxy-[1,3]thiazolo[5,4-b]pyridine

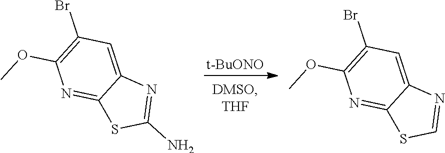

To a mixture of 6-bromo-5-methoxy-[1,3]thiazolo[5,4-b]pyridin-2-amine (1.3 g, 4.99 mmol) and DMSO (312.4 mg, 3.99 mmol) in THE (20.0 mL) was added dropwise 2-methyl-2-propylnitrite (1.5 g, 15.00 mmol) at room temperature. The resulting mixture was stirred at room temperature for 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 6-bromo-5-methoxy-[1,3]thiazolo[5,4-b]pyridine (800.0 mg, 65%) as a yellow solid. LCMS (ESI, m/z): [M+H]⁺=244.9

### Step 3: Synthesis of 6-chloro-3-[5-methoxy-[1,3]thiazolo[5,4-b]pyridin-6-yl]-1-[[2-(trimethylsilyl)ethoxy]methyl]pyrrolo[2,3-b]pyridine

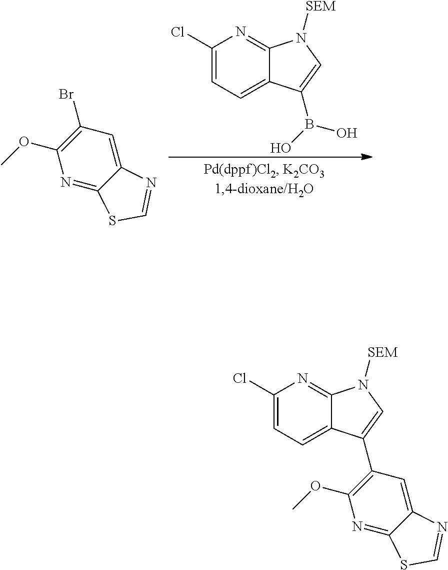 [SMILES: COc1nc2scnc2cc1Br ; C[Si](C)(C)CCOCn1cc(B(O)O)c2ccc(Cl)nc21 ; COc1nc2scnc2cc1-c1cn(COCC[Si](C)(C)C)c2nc(Cl)ccc12]

To a mixture of 6-bromo-5-methoxy-[1,3]thiazolo[5,4-b]pyridine (400.0 mg, 1.63 mmol) and 6-chloro-1-((2-(trimethylsilyl)ethoxy)methyl)-1H-pyrrolo[2,3-b]pyridin-3-ylboronic acid (1.0 g, 2.44 mmol) in dioxane/H₂O (10.0/1.0 mL) was added K₂CO₃ (676.6 mg, 4.89 mmol) and Pd(dppf)Cl₂ (119.4 mg, 0.16 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/1, v/v) to afford 6-chloro-3-[5-methoxy-[1,3]thiazolo[5,4-b]pyridin-6-yl]-1-[[2-(trimethylsilyl)ethoxy]methyl]pyrrolo[2,3-b]pyridine (370.0 mg, 50%) as a purple solid. LCMS (ESI, m/z): [M+H]⁺=447.1

### Step 4: Synthesis of (1S,2S)-2-fluoro-N-(3-[5-methoxy-[1,3]thiazolo[5,4-b]pyridin-6-yl]-1-[[2-(trimethylsilyl)ethoxy]methyl]pyrrolo[2,3-b]pyridin-6-yl)cyclopropane-1-carboxamide

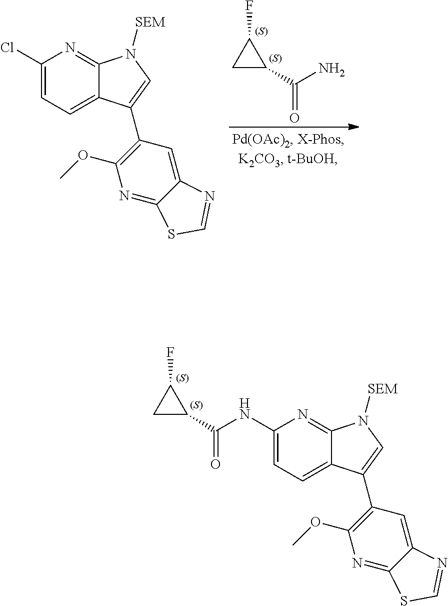

To a mixture of 6-chloro-3-[5-methoxy-[1,3]thiazolo[5,4-b]pyridin-6-yl]-1-[[2-(trimethylsilyl)ethoxy] methyl]pyrrolo[2,3-b]pyridine (350.0 mg, 0.78 mmol) and (1S,2S)-2-fluorocyclopropane-1-carboxamide (403.5 mg, 3.91 mmol) in BuOH (5.0 mL) was added Pd(OAc)₂ (35.1 mg, 0.15 mmol), K₂CO₃ (324.6 mg, 2.34 mmol) and X-Phos (37.3 mg, 0.07 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-(3-[5-methoxy-[1,3]thiazolo[5,4-b]pyridin-6-yl]-1-[[2-(trimethylsilyl)ethoxy]methyl]pyrrolo[2,3-b]pyridin-6-yl)cyclopropane-1-carboxamide (50.0 mg, 12%) as a green solid. LCMS (ESI, m/z): [M+H]⁺=514.2

### Step 5: Synthesis of (1S,2S)-2-fluoro-N-(3-[5-methoxy-[1,3]thiazolo[5,4-b]pyridin-6-yl]-1H-pyrrolo[2,3-b]pyridin-6-yl)cyclopropane-1-carboxamide (Compound 160)

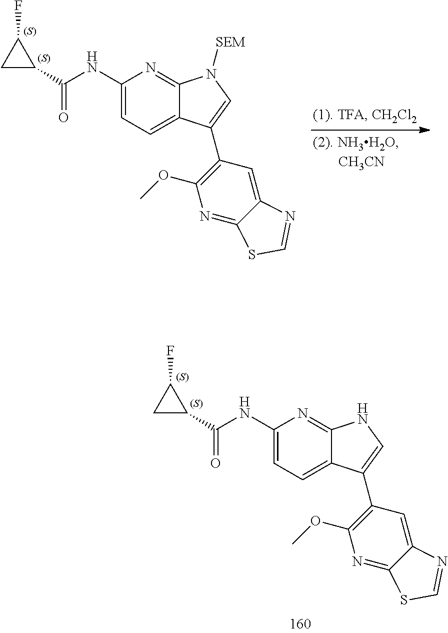

To a solution of (1S,2S)-2-fluoro-N-(3-[5-methoxy-[1,3]thiazolo[5,4-b]pyridin-6-yl]-1-[[2-(trimethylsilyl)ethoxy]methyl]pyrrolo[2,3-b]pyridin-6-yl)cyclopropane-1-carboxamide (40.0 mg, 0.07 mmol) in DCM (1.0 mL) was added TFA (1.0 mL) at room temperature. The resulting mixture was stirred at room temperature for 16 h. The resulting mixture was concentrated under reduced pressure. To the above residue was added ACN (1.0 mL) and NH₃·H₂O (2.0 mL). The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 37% B to 46% B in 8 min, 254 nm) to afford (1S,2S)-2-fluoro-N-(3-[5-methoxy-[1,3]thiazolo[5,4-b]pyridin-6-yl]-1H-pyrrolo[2,3-b]pyridin-6-yl)cyclopropane-1-carboxamide (Compound 160) (9.2 mg, 31%) LCMS (ESI, m/z): [M+H]⁺=384.1. ¹H NMR (300 MHz, DMSO-d₆): δ 11.81 (s, 1H), 10.68 (s, 1H), 9.33 (s, 1H), 8.54 (s, 1H), 8.13 (d, J=8.7 Hz, 1H), 7.95 (d, J=8.7 Hz, 1H), 7.80 (d, J=1.5 Hz, 1H), 5.10-4.80 (m, 1H), 4.05 (s, 3H), 2.25-2.15 (m, 1H), 1.78-1.60 (m, 1H), 1.27-1.10 (m, 1H).

### Example S157: Synthesis of 1-(3-(4-cyclopropoxy-2-methoxypyridin-3-yl)-1H-pyrrolo[2,3-b]pyridin-6-yl)-3-(2-(dimethylamino)ethyl)urea (Compound 161)

### Step 1: Synthesis of 1-[3-(4-cyclopropoxy-2-methoxypyridin-3-yl)-1-{[2-(trimethylsilyl)ethoxy]methyl}pyrrolo[2,3-b]pyridin-6-yl]-3-[2-(dimethylamino)ethyl]urea

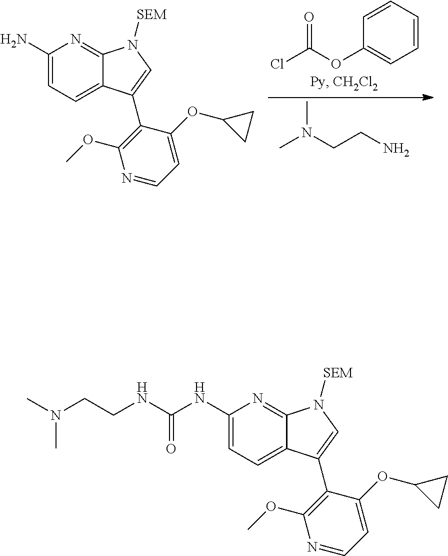

To a solution of 3-(4-cyclopropoxy-2-methoxypyridin-3-yl)-1-{[2-(trimethylsilyl)ethoxy]methyl}pyrrolo[2,3-b]pyridin-6-amine (180.0 mg, 0.42 mmol) in DCM (13.0 mL) was added pyridine (133.5 mg, 1.69 mmol) and phenyl chloroformate ((132.1 mg, 0.84 mmol) at room temperature. The resulting mixture was stirred at room temperature for 2 h. The resulting mixture was concentrated under reduced pressure. To the residue in pyridine (13.0 mL) was added (2-aminoethyl)dimethylamine (372.0 mg, 4.22 mmol) at room temperature. The resulting mixture was stirred at 60° C. for 16 h. After the reaction was completed, the resulting mixture was concentrated under reduced pressure. The residue was purified by flash column chromatography with CH₂Cl₂/MeOH (9/1, v/v) to afford 1-[3-(4-cyclopropoxy-2-methoxypyridin-3-yl)-1-{[2-(trimethylsilyl)ethoxy]methyl}pyrrolo[2,3-b]pyridin-6-yl]-3-[2-(dimethylamino)ethyl]urea (180.0 mg, 79%) as a white solid. LCMS (ESI, m/z): [M+H]⁺=541.3.

### Step 2: Synthesis of 1-(3-(4-cyclopropoxy-2-methoxypyridin-3-yl)-1H-pyrrolo[2,3-b]pyridin-6-yl)-3-(2-(dimethylamino)ethyl)urea (Compound 161)

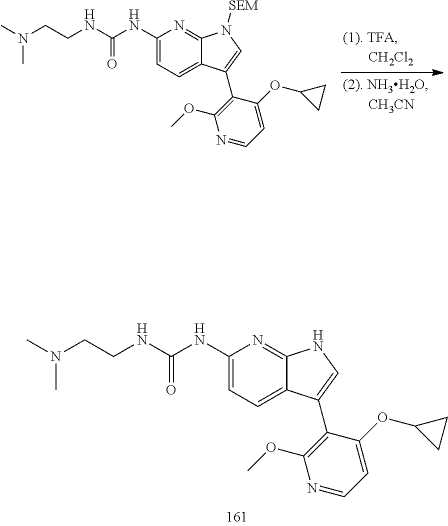 [SMILES: COc1nccc(OC2CC2)c1-c1cn(COCC[Si](C)(C)C)c2nc(NC(=O)NCCN(C)C)ccc12 ; COc1nccc(OC2CC2)c1-c1c[nH]c2nc(NC(=O)NCCN(C)C)ccc12]

To a solution of 1-[3-(4-cyclopropoxy-2-methoxypyridin-3-yl)-1-{[2-(trimethylsilyl)ethoxy]methyl}pyrrolo[2,3-b]pyridin-6-yl]-3-[2-(dimethylamino)ethyl]urea (180.0 mg, 0.33 mmol) in CH₂Cl₂ (8.0 mL) was added TFA (8.0 mL) at room temperature. The resulting mixture was stirred at room temperature for 4 h. The resulting mixture was concentrated under reduced pressure. To the residue in CH₃CN (8.0 mL) was added NH₃·H₂O (8.0 mL) at room temperature. The resulting mixture was stirred at room temperature for 16 h. After the reaction was completed, the resulting mixture was concentrated under reduced pressure. The residue was purified by Prep-HPLC with the following conditions Column (XBridge Prep OBD C18 Column, 30×150 mm, 5 m; Mobile Phase A: Water (10 mmol/L NH₄HCO₃). Mobile Phase B: ACN; Flow rate: 60 mL/min; Gradient: 20% B to 36% B in 8 min; 254 nm) to afford 1-(3-(4-cyclopropoxy-2-methoxypyridin-3-yl)-1H-pyrrolo[2,3-b]pyridin-6-yl)-3-(2-(dimethylamino)ethyl)urea (Compound 161) (49.6 mg, 36%) as a white solid. LCMS (ESI, m/z): [M+H]⁺=411.4. ¹H NMR (400 MHz, DMSO-d₆): δ 11.40 (s, 1H), 9.16 (s, 1H), 8.43 (s, 1H), 8.06 (d, J=6.0 Hz, 1H), 7.49 (d, J=8.8 Hz, 1H), 7.23 (d, J=2.0 Hz, 1H), 7.13 (d, J=6.0 Hz, 1H), 7.00 (d, J=8.4 Hz, 1H), 3.94-3.90 (m, 1H), 3.81 (s, 3H), 3.34-3.28 (m, 2H), 2.42-2.34 (m, 2H), 2.21 (s, 6H), 0.81-0.76 (m, 2H), 0.65-0.61 (m, 2H).

### Example S158: Synthesis of (1S,2S)—N-[3-(4-ethoxy-5-fluoro-2-methoxypyridin-3-yl)-1H-pyrrolo[2,3-b]pyridin-6-yl]-2-fluorocyclopropane-1-carboxamide (Compound 162)

### Step 1: Synthesis of 5-fluoro-3-iodo-2-methoxypyridin-4-ol

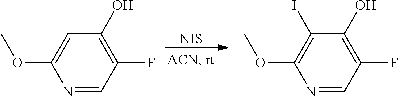

To a solution of 5-fluoro-2-methoxypyridin-4-ol (400.0 mg, 2.79 mmol) in MeCN (5.0 mL) was added NIS (628.8 mg, 2.79 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 5-fluoro-3-iodo-2-methoxypyridin-4-ol (450.0 mg, crude) as yellow solid. LCMS (ESI, m/z): [M+H]⁺=269.9

### Step 2: Synthesis of 4-ethoxy-5-fluoro-3-iodo-2-methoxypyridine

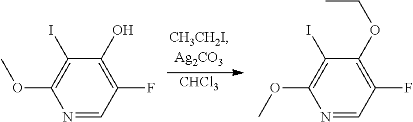

To a mixture of 5-fluoro-3-iodo-2-methoxypyridin-4-ol (700.0 mg, crude) and Ag₂CO₃ (2.8 g, 10.40 mmol) in CHCl₃ (15.0 mL) was added CH₃CH₂I (1.5 g, 10.40 mmol) at room temperature. The resulting mixture was stirred at room temperature for 16 h. After the reaction was completed, the resulting mixture was filtered. The filtrate was concentrated under reduced pressure. The residue was purified by flash column chromatography with petroleum ether/ethyl acetate (3/1, v/v) to afford 4-ethoxy-5-fluoro-3-iodo-2-methoxypyridine (700.0 mg, 90%) as a yellow solid. LCMS (ESI, m/z): [M+H]⁺=298.0.

### Step 3: Synthesis of 3-(6-chloro-1-[[2-(trimethylsilyl)ethoxy]methyl]pyrrolo[2,3-b]pyridin-3-yl)-4-ethoxy-5-fluoro-2-methoxypyridine

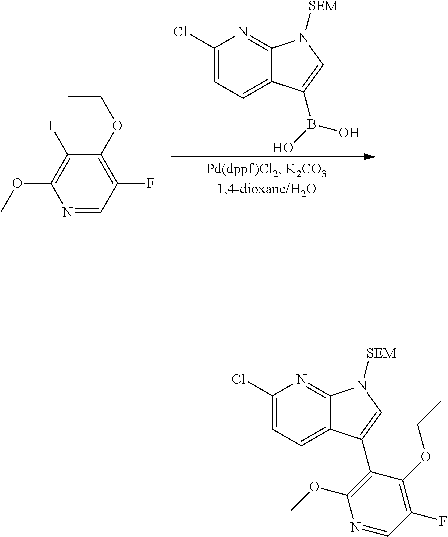 [SMILES: CCOc1c(F)cnc(OC)c1I ; C[Si](C)(C)CCOCn1cc(B(O)O)c2ccc(Cl)nc21 ; CCOc1c(F)cnc(OC)c1-c1cn(COCC[Si](C)(C)C)c2nc(Cl)ccc12]

To a mixture of 4-ethoxy-5-fluoro-3-iodo-2-methoxypyridine (200.0 mg, 0.67 mmol) and 6-chloro-1-[[2-(trimethylsilyl)ethoxy]methyl]pyrrolo[2,3-b]pyridin-3-ylboronic acid (263.9 mg, 0.80 mmol) in dioxane/H₂O (5.0/0.5 mL) was added Pd(PPh₃)₄ (155.6 mg, 0.13 mmol) and K₂CO₃ (279.1 mg, 2.02 mmol) at room temperature under N₂. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-(6-chloro-1-[[2-(trimethylsilyl)ethoxy]methyl]pyrrolo[2,3-b]pyridin-3-yl)-4-ethoxy-5-fluoro-2-methoxypyridine (140.0 mg, 46%) as a yellow solid. LCMS (ESI, m/z): [M+H]⁺=452.1

### Step 4: Synthesis of (1S,2S)—N-[3-(4-ethoxy-5-fluoro-2-methoxypyridin-3-yl)-1-[[2-(trimethylsilyl)ethoxy]methyl]pyrrolo[2,3-b]pyridin-6-yl]-2-fluorocyclopropane-1-carboxamide

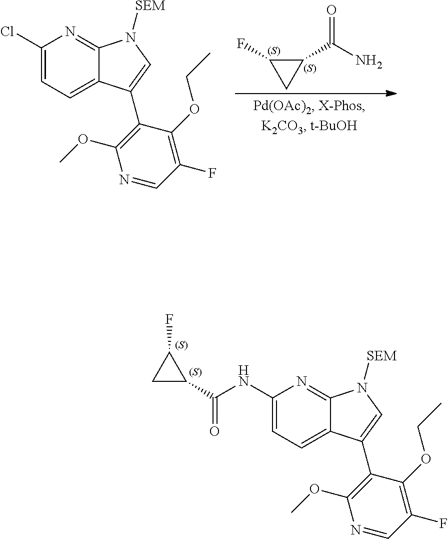

To a mixture of 3-(6-chloro-1-[[2-(trimethylsilyl)ethoxy]methyl]pyrrolo[2,3-b]pyridin-3-yl)-4-ethoxy-5-fluoro-2-methoxypyridine (100.0 mg, 0.22 mmol) and (1S,2S)-2-fluorocyclopropane-1-carboxamide (114.0 mg, 1.10 mmol) in t-BuOH (4.0 mL) was added X-Phos (42.1 mg, 0.08 mmol), Pd(OAc)₂ (9.93 mg, 0.044 mmol) and K₂CO₃ (91.7 mg, 0.66 mmol) at room temperature under N₂. The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-[3-(4-ethoxy-5-fluoro-2-methoxypyridin-3-yl)-1-[[2-(trimethylsilyl)ethoxy]methyl]pyrrolo[2,3-b]pyridin-6-yl]-2-fluorocyclopropane-1-carboxamide (80.0 mg, 69%) as a yellow solid. LCMS (ESI, m/z): [M+H]⁺=519.2

### Step 5: Synthesis of (1S,2S)—N-[3-(4-ethoxy-5-fluoro-2-methoxypyridin-3-yl)-1H-pyrrolo[2,3-b]pyridin-6-yl]-2-fluorocyclopropane-1-carboxamide (Compound 162)

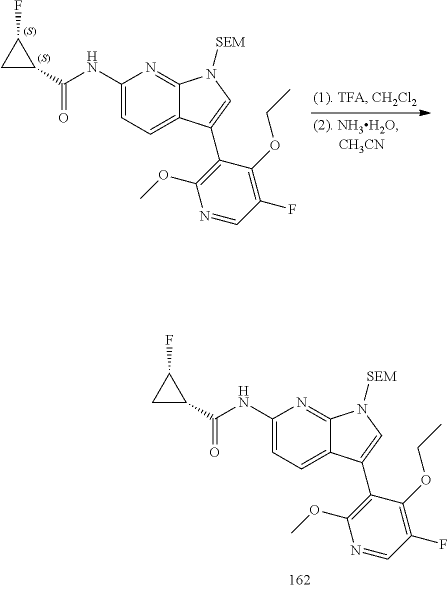

To a solution of (1S,2S)—N-[3-(4-ethoxy-5-fluoro-2-methoxypyridin-3-yl)-1-[[2-(trimethylsilyl)ethoxy]methyl]pyrrolo[2,3-b]pyridin-6-yl]-2-fluorocyclopropane-1-carboxamide (70.0 mg, 0.13 mmol) in DCM (2.0 mL) was added TFA (2.0 mL). The resulting mixture was stirred at room temperature for 2 h. The resulting mixture was concentrated under vacuum. To the above residue was added ACN (2.0 mL) and NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um, Mobile Phase A: Water (10 mmol/L NH₄HCO₃, Mobile Phase B ACN; Flow rate: 60 mL/min; Gradient: 39% B to 49% B in 9 min, 254/220 nm) to afford (1S,2S)—N-[3-(4-ethoxy-5-fluoro-2-methoxypyridin-3-yl)-1H-pyrrolo[2,3-b]pyridin-6-yl]-2-fluorocyclopropane-1-carboxamide (Compound 162) (5.4 mg, 10%) as a white solid. LCMS (ESI, m/z): [M+H]⁺=389.2. ¹H NMR (300 MHz, DMSO-d₆): δ 11.71 (s, 1H), 10.65 (s, 1H), 8.11 (d, J=2.7 Hz, 1H), 7.87 (d, J=8.4 Hz, 1H), 7.73 (d, J=8.7 Hz, 1H), 7.49 (d, J=2.7 Hz, 1H), 5.05-4.81 (m, 1H), 4.07-4.00 (m, 2H), 3.35 (s, 3H), 2.31-2.19 (m, 1H), 1.72-1.62 (m, 1H), 1.22-1.18 (m, 1H), 1.11-1.02 (m, 3H).

### Example S159: Synthesis of 1-(3-(5,7-dimethoxythiazolo[4,5-b]pyridin-6-yl)-1H-pyrrolo[2,3-b]pyridin-6-yl)-3-(2-(dimethylamino)ethyl)urea diformate (Compound 163)

### Step 1: Synthesis of 1-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-3-(2-(dimethylamino)ethyl)urea

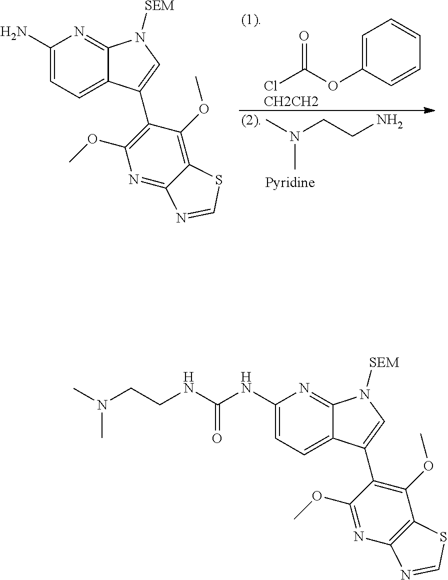

To a solution of 3-{5,7-dimethoxy-[1,3]thiazolo[4,5-b]pyridin-6-yl}-1-{[2-(trimethylsilyl)ethoxy]methyl}pyrrolo[2,3-b]pyridin-6-amine (150.0 mg, 0.33 mmol) in CH₂C₂ (2.0 mL) was added phenyl carbonochloridate (77.0 mg, 0.49 mmol) and pyridine (1.0 mL) at 0° C. The resulting mixture was stirred at 0° C. for 1 h. Then a solution of (2-aminoethyl)dimethylamine (260.6 mg, 2.95 mmol) in pyridine (3.0 mL) was added dropwise to the mixture at 0° C. The resulting mixture was stirred at 60° C. for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford 1-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-3-(2-(dimethylamino)ethyl)urea (100.0 mg, 86%) as a white solid. LCMS (ESI, m/z): [M+H]⁺=572.2.

### Step 2: Synthesis of 1-(3-(5,7-dimethoxythiazolo[4,5-b]pyridin-6-yl)-1H-pyrrolo[2,3-b]pyridin-6-yl)-3-(2-(dimethylamino)ethyl)urea diformate (Compound 163)

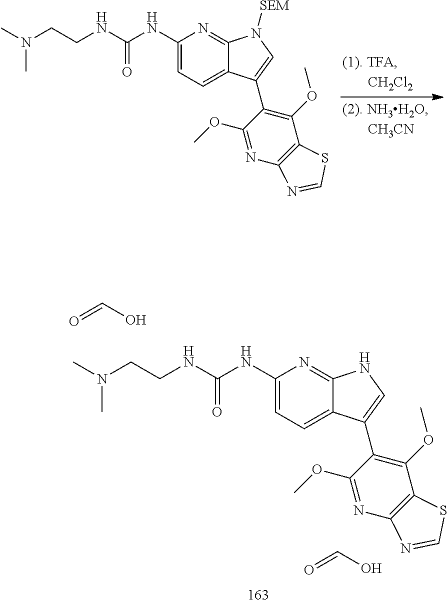

To a solution of 1-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-3-(2-(dimethylamino)ethyl)urea (100.0 mg, 0.21 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L FA), Mobile Phase B: MeOH; Flow rate: 25 mL/min; Gradient: 26% B to 26% B in 15 min; 254 nm) to afford 1-(3-(5,7-dimethoxythiazolo[4,5-b]pyridin-6-yl)-1H-pyrrolo[2,3-b]pyridin-6-yl)-3-(2-(dimethylamino)ethyl)urea diformate (Compound 163) (16.5 mg, 14%) as a white solid. LCMS (ESI, m/z): [M+H]⁺=442.3. ¹H NMR (400 MHz, DMSO-d₆): δ 11.56 (s, 1H), 9.56 (s, 1H), 9.19 (s, 1H), 8.43 (s, 1H), 8.24 (s, 2H), 7.63 (d, J=8.8 Hz, 1H), 7.36 (s, 1H), 7.06 (d, J=8.4 Hz, 1H), 3.92-3.89 (m, 6H), 3.39-3.33 (m, 2H), 2.51-2.45 (m, 2H), 2.25 (s, 6H).

### Example S160: Synthesis of 1-[3-(2,6-dimethoxyphenyl)-1H-pyrrolo[2,3-b]pyridin-6-yl]-3-[(2S)-3-(dimethylamino)-2-fluoropropyl]urea and 1-[3-(2,6-dimethoxyphenyl)-1H-pyrrolo[2,3-b]pyridin-6-yl]-3-[(2R)-3-(dimethylamino)-2-fluoropropyl]urea (Compound 164 and Compound 165)

### Step 1: Synthesis of methyl 2-(dibenzylamino)-3-hydroxypropanoate

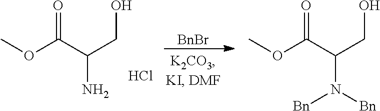

To a solution of methyl 2-amino-3-hydroxypropanoate hydrochloride (10.0 g, 64.28 mmol) in DMF (600.0 mL) was added KI (5.3 g, 32.14 mmol), K₂CO₃ (26.7 g, 192.83 mmol) and benzyl bromide (23.1 g, 134.98 mmol) dropwise at room temperature. The resulting mixture was stirred at room temperature for 16 h. After the reaction was completed, the resulting mixture was concentrated under reduced pressure. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (11/4, v/v) to afford methyl 2-(dibenzylamino)-3-hydroxypropanoate (15.1 g, 78%) as a colorless oil. LCMS (ESI, m/z): [M+H]⁺=300.2.

### Step 2: Synthesis of methyl 3-(dibenzylamino)-2-fluoropropanoate

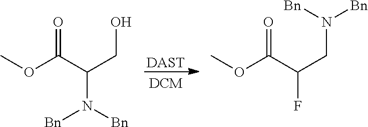

To a solution of methyl 2-(dibenzylamino)-3-hydroxypropanoate (7.0 g, 23.38 mmol) in DCM (300.0 mL) was added dropwise DAST (9.4 g, 58.46 mmol) at −40° C. under N₂. The reaction mixture was stirred at room temperature for 16 h. After the reaction was completed. The reaction mixture was quenched with sat. NH₄Cl (aq.) at 0° C. The resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/2, v/v) to afford methyl 3-(dibenzylamino)-2-fluoropropanoate (4.9 g, 68%) as a light yellow oil. LCMS (ESI, m/z): [M+H]⁺=302.1.

### Step 3: Synthesis of 3-(dibenzylamino)-2-fluoropropan-1-ol

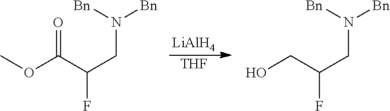 [SMILES: COC(=O)C(F)CN(Cc1ccccc1)Cc1ccccc1 ; "LiAlH4" ; OCC(F)CN(Cc1ccccc1)Cc1ccccc1 ; "THF"]

To a solution of LiAlH₄ (1.9 g, 49.77 mmol) in THE (200.0 mL) was added dropwise a solution of methyl 3-(dibenzylamino)-2-fluoropropanoate (5 g, 16.59 mmol) in THE at 0° C. under N₂. The resulting mixture was stirred at 0° C. for 1 h under N₂. After the reaction was completed. The reaction was quenched with water at 0° C. The pH value of the mixture was adjusted to 5 with HCl (aq.). The resulting mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/3, v/v) to afford 3-(dibenzylamino)-2-fluoropropan-1-ol (3.4 g, 74%) as a white solid. LCMS (ESI, m/z): [M+H]⁺=274.2.

### Step 4: Synthesis of 3-(dibenzylamino)-2-fluoropropyl methanesulfonate

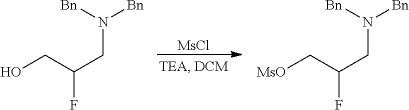 [SMILES: "MsCl" ; OCC(F)CN(Cc1ccccc1)Cc1ccccc1 ; CS(=O)(=O)OCC(F)CN(Cc1ccccc1)Cc1ccccc1]

To a solution of 3-(dibenzylamino)-2-fluoropropan-1-ol (5.2 g, 19.02 mmol) in CH₂Cl₂ (300.0 mL) was added TEA (2.9 g, 28.54 mmol) and methanesulfonyl chloride (2.4 g, 20.93 mmol) at 0° C. The resulting mixture was stirred at room temperature for 6 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure to afford 3-(dibenzylamino)-2-fluoropropyl methanesulfonate (6.6 g, crude) as a yellow oil. LCMS (ESI, m/z): [M+H]⁺=352.1.

### Step 5: Synthesis of [3-(dibenzylamino)-2-fluoropropyl]dimethylamine

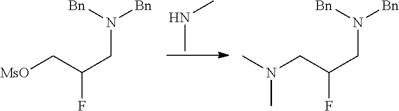

The mixture of 3-(dibenzylamino)-2-fluoropropyl methanesulfonate (4.0 g, crude) in dimethylamine/water (120.0 mL, 30%) was stirred at 60° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford [3-(dibenzylamino)-2-fluoropropyl]dimethylamine (3.3 g, crude) as a light yellow oil. LCMS (ESI, m/z): [M+H]⁺=301.2.

### Step 6: Synthesis of (3-amino-2-fluoropropyl)dimethylamine

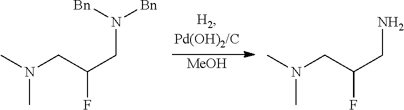

To a solution of [3-(dibenzylamino)-2-fluoropropyl]dimethylamine (3.8 g, 12.65 mmol) in MeOH (200.0 mL) was added Pd(OH)₂/C (1.1 g, wet) at room temperature. The resulting mixture was stirred at 60° C. for 16 h under H₂. After the reaction was completed, the resulting mixture was filtered. The filtrate was concentrated under reduced pressure to afford (3-amino-2-fluoropropyl)dimethylamine (1.4 g, crude) as a colorless oil. LCMS (ESI, m/z): [M+H]⁺=121.1.

### Step 7: Synthesis of 1-[3-(2,6-dimethoxyphenyl)-1-{[2-(trimethylsilyl)ethoxy]methyl}pyrrolo[2,3-b]pyridin-6-yl]-3-[3-(dimethylamino)-2-fluoropropyl]urea

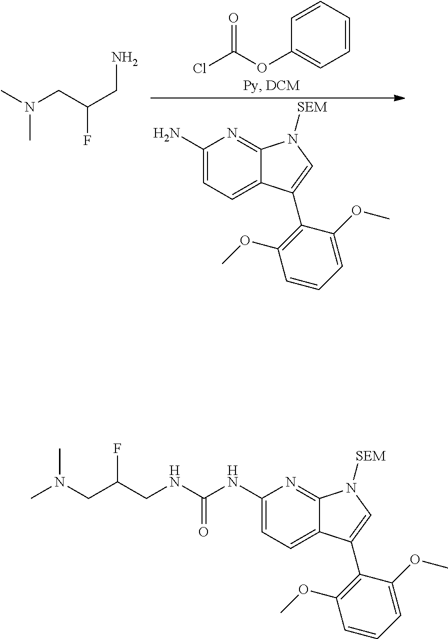

To a solution of 3-(2,6-dimethoxyphenyl)-1-{[2-(trimethylsilyl)ethoxy]methyl}pyrrolo[2,3-b]pyridin-6-amine (300.0 mg, 0.75 mmol) in DCM (12.0 mL) was added pyridine (237.6 mg, 3.00 mmol) and phenyl chloroformate (235.1 mg, 1.50 mmol) at room temperature. The resulting mixture was stirred at room temperature for 3 h. The resulting mixture was concentrated under reduced pressure. To the above residue was added (3-amino-2-fluoropropyl)dimethylamine (902.3 mg, 7.51 mmol) and pyridine (20.0 mL) at room temperature. The resulting mixture was stirred at 60° C. for 16 h. After the reaction was completed. the resulting mixture was concentrated under reduced pressure. The residue was purified by flash column chromatography with CH₂Cl₂/CH₃OH (13/1, v/v) to afford 1-[3-(2,6-dimethoxyphenyl)-1-{[2-(trimethylsilyl)ethoxy]methyl}pyrrolo[2,3-b]pyridin-6-yl]-3-[3-(dimethylamino)-2-fluoropropyl]urea (336.0 mg, 82%) as a light yellow oil. LCMS (ESI, m/z): [M+H]⁺=546.3.

### Step 8: Synthesis of 1-[3-(2,6-dimethoxyphenyl)-1H-pyrrolo[2,3-b]pyridin-6-yl]-3-[3-(dimethylamino)-2-fluoropropyl]urea

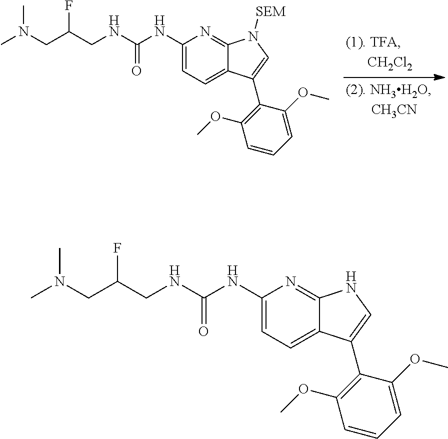

To a solution of 1-[3-(2,6-dimethoxyphenyl)-1-{[2-(trimethylsilyl)ethoxy]methyl}pyrrolo[2,3-b]pyridin-6-yl]-3-[3-(dimethylamino)-2-fluoropropyl]urea (300.0 mg, 0.55 mmol) in CH₂Cl₂ (10.0 mL) was added TFA (10.0 mL) at room temperature. The resulting mixture was stirred at room temperature for 3 h. The resulting mixture was concentrated under reduced pressure. To the residue in CH₃CN (10.0 mL) was added NH₃·H₂O (10.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with acetonitrile/water (7/4, v/v) to afford 1-[3-(2,6-dimethoxyphenyl)-1H-pyrrolo[2,3-b]pyridin-6-yl]-3-[3-(dimethylamino)-2-fluoropropyl]urea (106.0 mg, 46%) as a colorless oil. LCMS (ESI, m/z): [M+H]⁺=416.2.

### Step 9: Synthesis of 1-[3-(2,6-dimethoxyphenyl)-1H-pyrrolo[2,3-b]pyridin-6-yl]-3-[(2S)-3-(dimethylamino)-2-fluoropropyl]urea and 1-[3-(2,6-dimethoxyphenyl)-1H-pyrrolo[2,3-b]pyridin-6-yl]-3-[(2R)-3-(dimethylamino)-2-fluoropropyl]urea (Compound 164 and Compound 165)

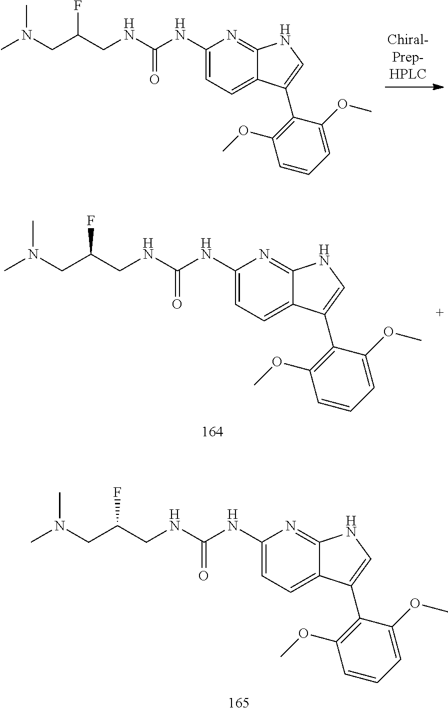

The racemic 1-[3-(2,6-dimethoxyphenyl)-1H-pyrrolo[2,3-b]pyridin-6-yl]-3-[3-(dimethylamino)-2-fluoropropyl]urea (90.0 mg, 0.22 mmol) was separated by Prep-Chiral-HPLC with the following conditions (Column: CHIRALPAK IA, 2×25 cm, 20 um; Mobile Phase A: Hex (0.5% 2M NH₃-MeOH)—HPLC, Mobile Phase B: EtOH—HPLC; Flow rate: 20 mL/min; Gradient: 50% B to 50% B in 14 min; 220/254 nm) to afford 1-[3-(2,6-dimethoxyphenyl)-1H-pyrrolo[2,3-b]pyridin-6-yl]-3-[3-(dimethylamino)-2-fluoropropyl]urea Enantiomer 1 (12.6 mg, 14%) as a white solid and 1-[3-(2,6-dimethoxyphenyl)-1H-pyrrolo[2,3-b]pyridin-6-yl]-3-[3-(dimethylamino)-2-fluoropropyl]urea Enantiomer 2 (16.9 mg, 18%) as a white solid. The absolute stereochemistry of Enantiomers 1 and 2 was not assigned. The two enantiomeric structures that could be obtained from chiral separation of the enantiomeric mixture as described above are shown as Compounds 164 and 165 in Table 1.

1-[3-(2,6-dimethoxyphenyl)-1H-pyrrolo[2,3-b]pyridin-6-yl]-3-[3-(dimethylamino)-2-fluoropropyl]urea Enantiomer 1: Retention Time 1: 7.811 min; LCMS (ESI, m/z): [M+H]⁺=416.1. ¹H NMR (300 MHz, CD₃OD): δ 7.59 (d, J=8.7 Hz, 1H), 7.31-7.26 (m, 1H), 7.21 (s, 1H), 6.75 (d, J=8.4 Hz, 2H), 6.68 (d, J=8.4 Hz, 1H), 5.00-4.80 (m, 1H), 3.77-3.71 (m, 7H), 3.69-3.50 (m, 1H), 2.78-2.71 (m, 1H), 2.69-2.50 (m, 1H), 2.36 (s, 6H).

1-[3-(2,6-dimethoxyphenyl)-1H-pyrrolo[2,3-b]pyridin-6-yl]-3-[3-(dimethylamino)-2-fluoropropyl]urea Enantiomer 2: Retention Time 2: 11.45 min; LCMS (ESI, m/z): [M+H]⁺=416.1. ¹H NMR (300 MHz, CD₃OD): δ 7.59 (d, J=8.4 Hz, 1H), 7.31-7.26 (m, 1H), 7.21 (s, 1H), 6.75 (d, J=8.4 Hz, 2H), 6.68 (d, J=8.4 Hz, 1H), 5.00-4.80 (m, 1H), 3.77-3.71 (m, 6H), 3.69-3.50 (m, 2H), 2.79-2.71 (m, 1H), 2.69-2.50 (m, 1H), 2.36 (s, 6H).

### Example S161: Synthesis of (1S,2S)-2-fluoro-N-[3-(7-fluoro-6-methoxy-3H-1,3-benzodiazol-5-yl)-1H-pyrrolo[2,3-b]pyridin-6-yl]cyclopropane-1-carboxamide (Compound 166)

### Step 1: Synthesis of 2-fluoro-3-methoxy-6-nitroaniline

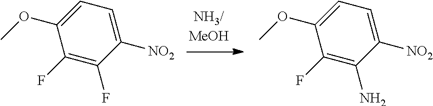

The solution of 2,3-difluoro-1-methoxy-4-nitrobenzene (2.0 g, 10.57 mmol) in NH₃/MeOH (20.0 mL, 7 mol/L) was stirred at 80° C. for 16 h. After the reaction was completed, the resulting mixture was concentrated under reduced pressure. The residue was purified by flash column chromatography with petroleum ether/ethyl acetate (5/1, v/v) to afford 2-fluoro-3-methoxy-6-nitroaniline (1.8 g, 91%) as a yellow solid. LCMS (ESI, m/z): [M+H]⁺=187.0

### Step 2: Synthesis of 4-bromo-2-fluoro-3-methoxy-6-nitroaniline

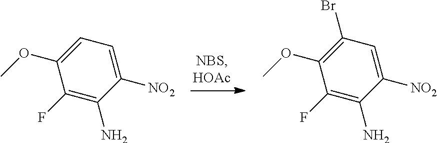

The mixture of 2-fluoro-3-methoxy-6-nitroaniline (1.5 g, 8.05 mmol) and NBS (2.1 g, 12.08 mmol) in AcOH (20.0 mL) was stirred at 90° C. for 4 h. After the reaction was completed, the reaction mixture was diluted with water and then filtered. The solid was washed with H₂O and then collected to afford 4-bromo-2-fluoro-3-methoxy-6-nitroaniline (2.5 g, crude) as a yellow solid. LCMS (ESI, m/z): [M+H]⁺=265.0

### Step 3: Synthesis of 6-bromo-4-fluoro-5-methoxy-1H-1,3-benzodiazole

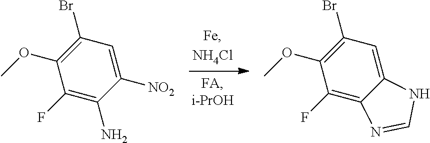

To a mixture of 4-bromo-2-fluoro-3-methoxy-6-nitroaniline (1.5 g, 5.66 mmol) and Fe (3.1 g, 56.60 mmol) in i-PrOH (10.0 mL) was added NH₄Cl (2.1 g, 39.62 mmol) and formic acid (10.0 mL) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (4/1, v/v) to afford 6-bromo-4-fluoro-5-methoxy-1H-1,3-benzodiazole (620.0 mg, 44%) as a brown solid. LCMS (ESI, m/z): [M+H]⁺=245.0.

### Step 4: Synthesis of 6-bromo-4-fluoro-5-methoxy-1-{[2-(trimethylsilyl)ethoxy]methyl}-1,3-benzodiazole

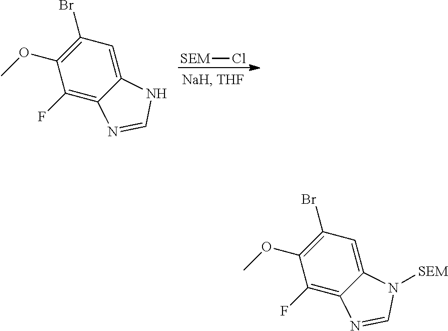 [SMILES: COc1c(Br)cc2[nH]cnc2c1F ; C[Si](C)(C)CCOCCl ; COc1c(Br)cc2c(ncn2COCC[Si](C)(C)C)c1F]

To a solution of 6-bromo-4-fluoro-5-methoxy-1H-1,3-benzodiazole (500.0 mg, 2.04 mmol) in THE (5.0 mL) was added NaH (146.8 mg, 60%) at 0° C. under N₂. The resulting mixture was stirred at 0° C. for 2 h. Then [2-(chloromethoxy)ethyl]trimethylsilane (510.2 mg, 3.06 mmol) was added to the mixture at 0° C. The resulting mixture was stirred at room temperature for additional 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (4/1, v/v) to afford 6-bromo-4-fluoro-5-methoxy-1-{[2-(trimethylsilyl)ethoxy]methyl}-1,3-benzodiazole (700.0 mg, 91%) as a yellow oil. LCMS (ESI, m/z): [M+H]⁺=375.0

### Step 5: Synthesis of 6-(6-chloro-1-{[2-(trimethylsilyl)ethoxy]methyl}pyrrolo[2,3-b]pyridin-3-yl)-4-fluoro-5-methoxy-1-{[2-(trimethylsilyl)ethoxy]methyl}-1,3-benzodiazole

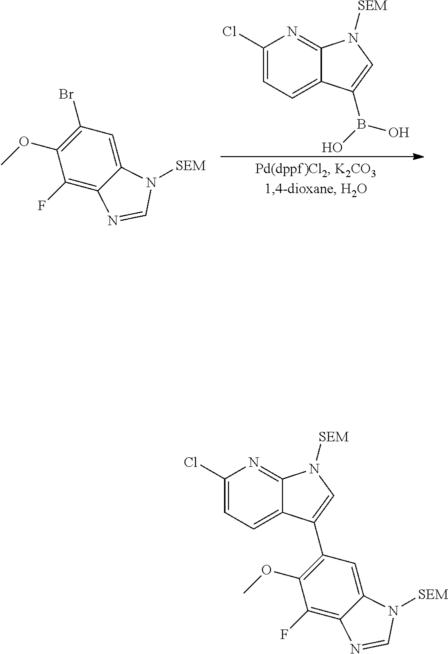 [SMILES: COc1c(Br)cc2c(ncn2COCC[Si](C)(C)C)c1F ; C[Si](C)(C)CCOCn1cc(B(O)O)c2ccc(Cl)nc21 ; COc1c(-c2cn(COCC[Si](C)(C)C)c3nc(Cl)ccc23)cc2c(ncn2COCC[Si](C)(C)C)c1F]

To a mixture of 6-bromo-4-fluoro-5-methoxy-1-{[2-(trimethylsilyl)ethoxy]methyl}-1,3-benzodiazole (350.0 mg, 0.93 mmol) and 6-chloro-1-{[2-(trimethylsilyl)ethoxy]methyl}pyrrolo[2,3-b]pyridin-3-ylboronic acid (609.2 mg, 1.86 mmol) in 1,4-dioxane/H₂O (5.0/0.5 mL) were added Pd(dppf)Cl₂ (136.4 mg, 0.18 mmol) and K₂CO₃ (386.6 mg, 2.79 mmol) at room temperature under N₂. The resulting mixture was stirred at 80° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) afford 6-(6-chloro-1-{[2-(trimethylsilyl)ethoxy]methyl}pyrrolo[2,3-b]pyridin-3-yl)-4-fluoro-5-methoxy-1-{[2-(trimethylsilyl)ethoxy]methyl}-1,3-benzodiazole (200.0 mg, 37%) as a yellow oil. LCMS (ESI, m/z): [M+H]⁺=577.2.

### Step 6: Synthesis of (1S,2S)-2-fluoro-N-[3-(7-fluoro-6-methoxy-3-{[2-(trimethylsilyl)ethoxy]methyl}-1,3-benzodiazol-5-yl)-1-{[2-(trimethylsilyl)ethoxy]methyl}pyrrolo[2,3-b]pyridin-6-yl]cyclopropane-1-carboxamide

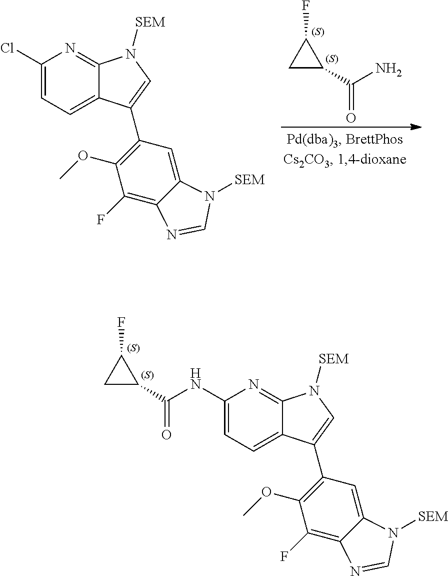

To a mixture of 6-(6-chloro-1-{[2-(trimethylsilyl)ethoxy]methyl}pyrrolo[2,3-b]pyridin-3-yl)-4-fluoro-5-methoxy-1-{[2-(trimethylsilyl)ethoxy]methyl}-1,3-benzodiazole (190.0 mg, 0.32 mmol) and (1S,2S)-2-fluorocyclopropane-1-carboxamide (169.6 mg, 1.64 mmol) in 1,4-dioxane (4.0 mL) were added Pd₂(dba)₃ (60.2 mg, 0.06 mmol), BrettPhos (70.7 mg, 0.13 mmol) and Cs₂CO₃ (321.7 mg, 0.98 mmol) at room temperature under N₂. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford (1S,2S)-2-fluoro-N-[3-(7-fluoro-6-methoxy-3-{[2-(trimethylsilyl)ethoxy]methyl}-1,3-benzodiazol-5-yl)-1-{[2-(trimethylsilyl)ethoxy]methyl}pyrrolo[2,3-b]pyridin-6-yl]cyclopropane-1-carboxamide (100.0 mg, 47%) as a yellow oil. LCMS (ESI, m/z): [M+H]⁺=644.3.

### Step 7: Synthesis of (1S,2S)-2-fluoro-N-[3-(7-fluoro-6-methoxy-3H-1,3-benzodiazol-5-yl)-1H-pyrrolo[2,3-b]pyridin-6-yl]cyclopropane-1-carboxamide (Compound 166)

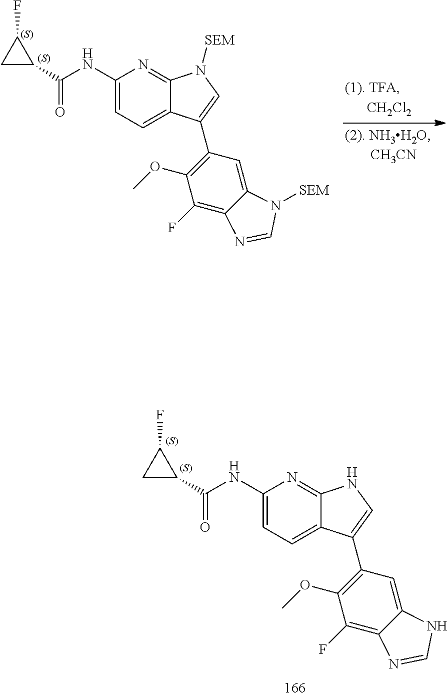

To a solution of (1S,2S)-2-fluoro-N-[3-(7-fluoro-6-methoxy-3-{[2-(trimethylsilyl)ethoxy]methyl}-1,3-benzodiazol-5-yl)-1-{[2-(trimethylsilyl)ethoxy]methyl}pyrrolo[2,3-b]pyridin-6-yl]cyclopropane-1-carboxamide (100.0 mg, 0.15 mmol) in DCM (1.0 mL) was added TFA (1.0 mL). The mixture was stirred at room temperature for 2 h. The resulting mixture was concentrated under reduced pressure. To the above residue was added NH₃·H₂O (2.0 mL) and ACN (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 14% B to 34% B in 9 min, 254 nm) to afford (1S,2S)-2-fluoro-N-[3-(7-fluoro-6-methoxy-3H-1,3-benzodiazol-5-yl)-1H-pyrrolo[2,3-b]pyridin-6-yl]cyclopropane-1-carboxamide (Compound 166) (5.2 mg, 9%) as a white solid. LCMS (ESI, m/z): [M+H]⁺=384.1. ¹H NMR (400 MHz, DMSO-d₆): δ 12.73-12.68 (br, 1H), 11.70 (s, 1H), 10.69 (s, 1H), 8.25 (s, 1H), 8.06 (d, J=8.4 Hz, 1H), 7.94 (d, J=8.0 Hz, 1H), 7.65 (s, 1H), 7.50 (s, 1H), 5.02-4.85 (m, 1H), 3.62 (s, 3H), 2.32-2.25 (m, 1H), 1.70-1.64 (m, 1H), 1.23-1.16 (m, 1H).

### Example S162: Synthesis of 1-[3-(4-cyclopropoxy-6-methoxypyrimidin-5-yl)-1H-pyrrolo[2,3-b]pyridin-6-yl]-3-{2-[2-(dimethylamino)ethoxy]ethyl}urea; formic acid (Compound 167)

### Step 1: Synthesis of 1-[3-(4-cyclopropoxy-6-methoxypyrimidin-5-yl)-1-{[2-(trimethylsilyl)ethoxy]methyl}pyrrolo[2,3-b]pyridin-6-yl]-3-{2-[2-(dimethylamino)ethoxy]ethyl}urea

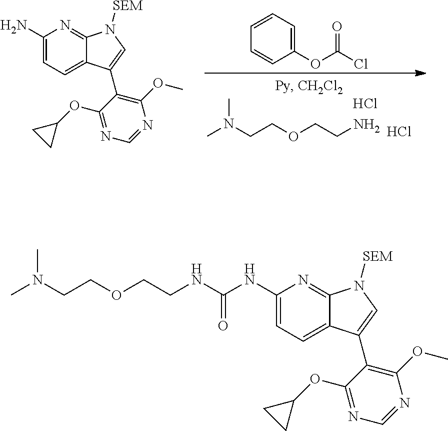

To a solution of 3-(4-cyclopropoxy-6-methoxypyrimidin-5-yl)-1-{[2-(trimethylsilyl)ethoxy]methyl}pyrrolo[2,3-b]pyridin-6-amine (200.0 mg, 0.47 mmol) in DCM (4.0 mL) was added phenyl chloroformate (87.9 mg, 0.56 mmol) and pyridine (148.0 mg, 1.87 mmol) at room temperature. The resulting mixture was stirred at room temperature for 1 h. The resulting mixture was concentrated under reduced pressure. To the residue in pyridine (4.0 mL) was added [2-(2-aminoethoxy)ethyl]dimethylamine (185.5 mg, 1.40 mmol) at room temperature. The resulting mixture was stirred at 60° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with CH₃CN/H₂O (80/20, v/v) to afford 1-[3-(4-cyclopropoxy-6-methoxypyrimidin-5-yl)-1-{[2-(trimethylsilyl)ethoxy]methyl}pyrrolo[2,3-b]pyridin-6-yl]-3-{2-[2-(dimethylamino)ethoxy]ethyl}urea (100.0 mg, 36%) as a yellow solid. LCMS (ESI, m/z): [M+H]⁺=586.3.

### Step 2: Synthesis of 1-[3-(4-cyclopropoxy-6-methoxypyrimidin-5-yl)-1H-pyrrolo[2,3-b]pyridin-6-yl]-3-{2-[2-(dimethylamino)ethoxy]ethyl}urea; formic acid (Compound 167)

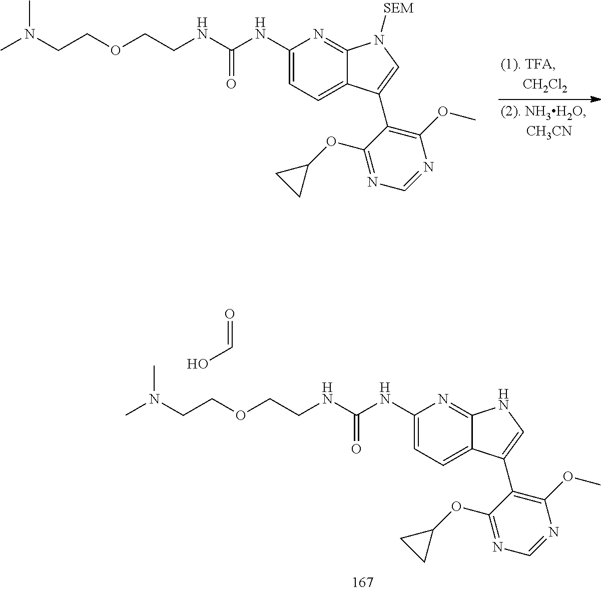

To a solution of 1-[3-(4-cyclopropoxy-6-methoxypyrimidin-5-yl)-1-{[2-(trimethylsilyl)ethoxy]methyl}pyrrolo[2,3-b]pyridin-6-yl]-3-{2-[2-(dimethylamino)ethoxy]ethyl}urea (100 mg, 0.17 mmol) in DCM (1.0 mL) was added TFA (1.0 mL) at room temperature. The resulting mixture was stirred at room temperature for 2 h. The resulting mixture was concentrated under reduced pressure. To the residue in ACN (1.0 mL) was added NH₃·H₂O (1.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 5% B to 30% B in 7 min, 254 nm) to afford 1-[3-(4-cyclopropoxy-6-methoxypyrimidin-5-yl)-1H-pyrrolo[2,3-b]pyridin-6-yl]-3-{2-[2-(dimethylamino)ethoxy]ethyl}urea; formic acid (Compound 167) (3.9 mg, 4%) as a white solid. LCMS (ESI, m/z): [M+H]⁺=456.2. ¹H NMR (400 MHz, DMSO-d₆): δ 11.74 (s, 1H), 9.25 (s, 1H), 8.70 (d, J=4.0 Hz, 1H), 8.50 (s, 1H), 8.22-8.19 (m, 1H) 7.58 (d, J=8.8 Hz, 1H), 7.35 (d, J=2.0 Hz, 1H), 6.99 (d, J=8.0 Hz, 1H), 4.37-4.34 (m, 1H), 3.91 (s, 3H), 3.63-3.59 (m, 2H), 3.54-3.45 (m, 2H), 3.39-3.34 (m, 2H), 2.68-2.60 (m, 1H), 2.34-2.14 (m, 7H), 0.77-0.69 (m, 2H), 0.65-0.61 (m, 2H).

### Example S163: Synthesis of 1-[3-(4-cyclopropoxy-6-methoxypyrimidin-5-yl)-1H-pyrrolo[2,3-b]pyridin-6-yl]-3-[(2R)-3-(dimethylamino)-2-fluoropropyl]urea and 1-[3-(4-cyclopropoxy-6-methoxypyrimidin-5-yl)-1H-pyrrolo[2,3-b]pyridin-6-yl]-3-[(2S)-3-(dimethylamino)-2-fluoropropyl]urea (Compound 168 and Compound 169)

### Step 1: Synthesis of 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea

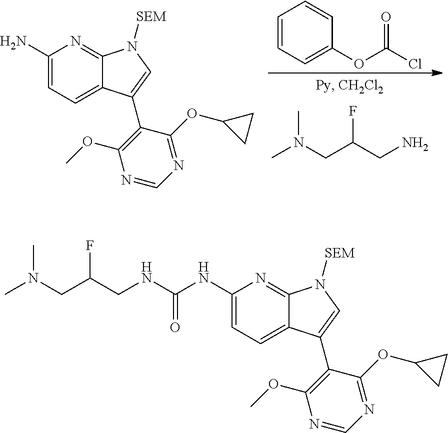

To a solution of 3-(4-cyclopropoxy-6-methoxypyrimidin-5-yl)-1-{[2-(trimethylsilyl)ethoxy]methyl}pyrrolo[2,3-b]pyridin-6-amine (190.0 mg, 0.44 mmol) in CH₂Cl₂ (4.0 mL) was added pyridine (140.6 mg, 1.78 mmol) and phenyl chloroformate (83.5 mg, 0.53 mmol) at room temperature. The resulting mixture was stirred at room temperature for 1 h. The resulting mixture was concentrated under reduced pressure. To the residue in pyridine (4.0 mL) was added (3-amino-2-fluoropropyl)dimethylamine (534.0 mg, 4.44 mmol) at room temperature. The resulting mixture was stirred at 60° C. for another 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum CH₂Cl₂/CH₃OH (95/5, v/v) to afford 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea (160.0 mg, 62%) as a colorless oil. LCMS (ESI, m/z): [M+H]⁺=574.3.

### Step 2: Synthesis of 1-[3-(4-cyclopropoxy-6-methoxypyrimidin-5-yl)-1H-pyrrolo[2,3-b]pyridin-6-yl]-3-[(2R)-3-(dimethylamino)-2-fluoropropyl]urea and 1-[3-(4-cyclopropoxy-6-methoxypyrimidin-5-yl)-1H-pyrrolo[2,3-b]pyridin-6-yl]-3-[(2S)-3-(dimethylamino)-2-fluoropropyl]urea (Compound 168 and Compound 169)

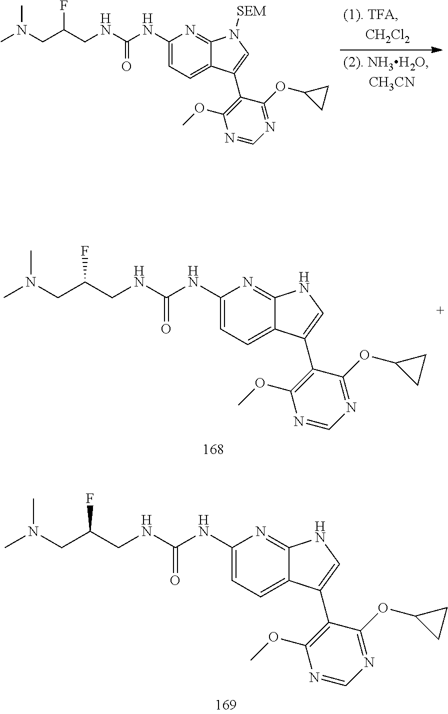 [SMILES: COc1ncnc(OC2CC2)c1-c1cn(COCC[Si](C)(C)C)c2nc(NC(=O)NCC(F)CN(C)C)ccc12 ; COc1ncnc(OC2CC2)c1-c1c[nH]c2nc(NC(=O)NC[C@@H](F)CN(C)C)ccc12 ; COc1ncnc(OC2CC2)c1-c1c[nH]c2nc(NC(=O)NC[C@H](F)CN(C)C)ccc12]

To a solution of 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea (150.0 mg, 0.26 mmol) in CH₂Cl₂ (4.0 mL) was added TFA (4.0 mL) at room temperature. The resulting mixture was stirred at room temperature for 4 h. The resulting mixture was concentrated under reduced pressure. To the residue in CH₃CN (4.0 mL) was added NH₃·H₂O (4.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was Prep-Chiral-HPLC with the following conditions (Column: CHIRAL ART Amylose-SA, 2×25 cm, 5 um; Mobile Phase A: Hex (0.5% 2M NH₃-MeOH)—HPLC, Mobile Phase B: EtOH—HPLC; Flow rate: 20 mL/min; Gradient: 50% B to 50% B in 10.5 min; 254 nm) to afford 1-[3-(4-cyclopropoxy-6-methoxypyrimidin-5-yl)-1H-pyrrolo[2,3-b]pyridin-6-yl]-3-[3-(dimethylamino)-2-fluoropropyl]urea Enantiomer 1 (10.2 mg, 8%) as a white solid and 1-[3-(4-cyclopropoxy-6-methoxypyrimidin-5-yl)-1H-pyrrolo[2,3-b]pyridin-6-yl]-3-[3-(dimethylamino)-2-fluoropropyl]urea Enantiomer 2 (11.0 mg, 9%) as a white solid. The absolute stereochemistry of Enantiomers 1 and 2 was not assigned. The two enantiomeric structures that could be obtained from chiral separation of the enantiomeric mixture as described above are shown as Compounds 168 and 169 in Table 1.

1-[3-(4-cyclopropoxy-6-methoxypyrimidin-5-yl)-1H-pyrrolo[2,3-b]pyridin-6-yl]-3-[3-(dimethylamino)-2-fluoropropyl]urea Enantiomer 1: Retention Time 1: 5.876 min; LCMS (ESI, m/z): [M+H]⁺=444.1. ¹H NMR (400 MHz, DMSO-d₆): δ 11.59 (s, 1H), 9.25 (s, 1H), 8.58 (s, 1H), 8.49 (s, 1H), 7.60 (d, J=8.4 Hz, 1H), 7.33 (s, 1H), 7.04 (d, J=8.4 Hz, 1H), 4.81-4.70 (m, 1H), 4.37-4.33 (m, 1H), 3.92 (s, 3H), 3.64-3.52 (m, 1H), 3.45-3.38 (m, 1H), 2.22 (s, 6H), 0.86-0.82 (m, 2H), 0.69-0.62 (m, 2H).

1-[3-(4-cyclopropoxy-6-methoxypyrimidin-5-yl)-1H-pyrrolo[2,3-b]pyridin-6-yl]-3-[3-(dimethylamino)-2-fluoropropyl]urea Enantiomer 2: Retention Time 2: 9.106 min; LCMS (ESI, m/z): [M+H]⁺=444.2. ¹H NMR (400 MHz, DMSO-d₆): δ 11.60 (s, 1H), 9.27 (s, 1H), 8.60 (s, 1H), 8.49 (s, 1H), 7.60 (d, J=8.4 Hz, 1H), 7.33 (d, J=2.4 Hz, 1H), 7.03 (d, J=8.8 Hz, 1H), 4.81-4.68 (m, 1H), 4.37-4.33 (m, 1H), 3.91 (s, 3H), 3.64-3.52 (m, 1H), 3.43-3.34 (m, 1H), 2.21 (s, 6H), 0.85-0.72 (m, 2H), 0.72-0.62 (m, 2H).

### Example S164: Synthesis of trans-2-(2-hydroxyethyl)-N-[3-(2-methoxyphenyl)-1H-pyrrolo[2,3-b]pyridin-6-yl]cyclopropane-1-carboxamide (Compound 170)

### Step 1: Synthesis of 2-(but-3-en-1-yloxy)tetrahydro-2H-pyran

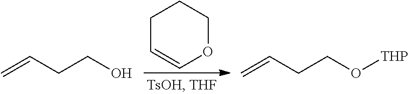

To a solution of 3-buten-1-ol (10.0 g, 138.68 mmol) in tetrahydrofuran (100.0 mL) was added 3,4-dihydro-2H-pyran (2.4 g, 13.88 mmol) and 4-methylbenzenesulfonic acid (17.5 g, 208.01 mmol) at 0° C. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (10/1, v/v) to afford 2-(but-3-en-1-yloxy)tetrahydro-2H-pyran (9.3 g, 43%) as a colorless oil.

### Step 2: Synthesis of Trans-ethyl 2-[2-(oxan-2-yloxy)ethyl]cyclopropane-1-carboxylate

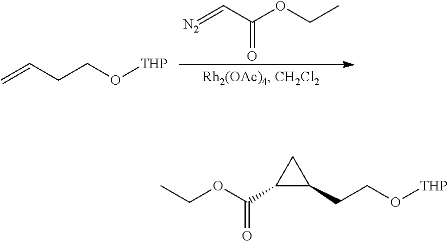

To a mixture of 2-(but-3-en-1-yloxy)tetrahydro-2H-pyran (800.0 mg, 5.11 mmol) and Rh₂ (OAc)₄ (22.63 mg, 0.05 mmol) in CH₂Cl₂ (5.0 mL) was added dropwise ethyl diazoacetate (1.1 g, 10.22 mmol) at room temperature during 8 h under N₂. The resulting mixture was stirred at room temperature for 16 h. After the reaction was completed, the mixture was concentrated under reduced pressure. The residue was purified by flash column chromatography with petroleum ether/ethyl acetate (5/1, v/v) to afford trans-ethyl 2-[2-(oxan-2-yloxy)ethyl]cyclopropane-1-carboxylate (300.0 mg, 24%) as a yellow oil. LCMS (ESI, m/z): [M+H]⁺=243.2.

### Step 3: Synthesis of trans-N-[3-(2-methoxyphenyl)-1-[[2-(trimethylsilyl)ethoxy]methyl]pyrrolo[2,3-b]pyridin-6-yl]-2-[2-(oxan-2-yloxy)ethyl]cyclopropane-1-carboxamide

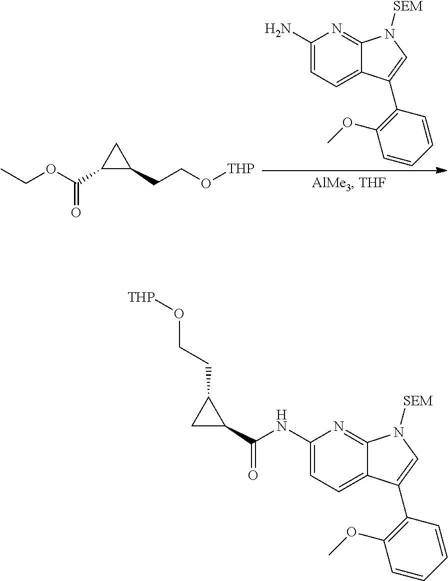

To a solution of trans-ethyl 2-(2-((tetrahydro-2H-pyran-2-yl)oxy)ethyl)cyclopropane-1-carboxylate (300.0 mg, 0.82 mmol) in THF (5.0 mL) was added 3-(2-methoxyphenyl)-1-((2-(trimethylsilyl)ethoxy)methyl)-1H-pyrrolo[2,3-b]pyridin-6-amine (590.15 mg, 2.43 mmol) and AlMe₃ (0.2 mL, 2 mol/L) at 0° C. The resulting mixture was stirred at 80° C. for 16 h. After the reaction was completed, the resulting mixture was concentrated under vacuum. The residue was purified by reverse flash column chromatography with MeOH/H₂O (5/80, v/v) to afford trans-N-[3-(2-methoxyphenyl)-1-[[2-(trimethylsilyl)ethoxy]methyl]pyrrolo[2,3-b]pyridin-6-yl]-2-[2-(oxan-2-yloxy)ethyl]cyclopropane-1-carboxamide (100.0 mg, 33%) as a yellow oil. LCMS (ESI, m/z): [M+H]⁺=566.3.

### Step 4: Synthesis of trans-2-(2-hydroxyethyl)-N-[3-(2-methoxyphenyl)-1H-pyrrolo[2,3-b]pyridin-6-yl]cyclopropane-1-carboxamide (Compound 170)

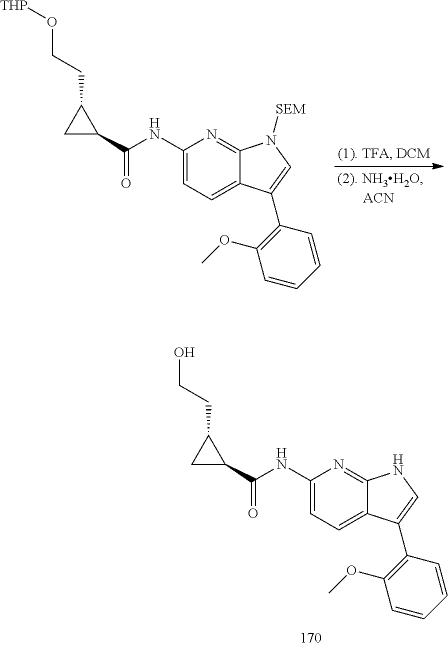

To a solution of trans-N-[3-(2-methoxyphenyl)-1-[[2-(trimethylsilyl)ethoxy]methyl]pyrrolo[2,3-b]pyridin-6-yl]-2-[2-(oxan-2-yloxy)ethyl]cyclopropane-1-carboxamide (100.0 mg, 0.17 mmol) in CH₂Cl₂ (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 36% B to 46% B in 8 min; 254 nm) to afford trans-2-(2-hydroxyethyl)-N-[3-(2-methoxyphenyl)-1H-pyrrolo[2,3-b]pyridin-6-yl]cyclopropane-1-carboxamide (Compound 170) (8.7 mg, 14%) as a white solid. LCMS (ESI, m/z): [M+H]⁺=352.1. ¹H NMR (300 MHz, DMSO-d₆): δ 11.55 (d, J=2.4 Hz, 1H), 10.50 (s, 1H), 7.99-7.89 (m, 2H), 7.57-7.53 (m, 2H), 7.29-7.24 (m, 1H), 7.11-7.00 (m, 2H), 4.55-4.52 (m, 1H), 3.81 (s, 3H), 3.54-3.48 (m, 2H), 1.86-1.84 (m, 1H), 1.48-1.42 (m, 2H), 1.34-1.28 (m, 1H), 1.04-1.01 (m, 1H), 0.71-0.68 (m, 1H).

### Example S165: Synthesis of (1S,2R)—N-(3-(2,6-dimethoxyphenyl)-1H-pyrrolo[2,3-b]pyridin-6-yl)-2-(piperazin-1-ylmethyl)cyclopropane-1-carboxamide and (1R,2S)—N-(3-(2,6-dimethoxyphenyl)-1H-pyrrolo[2,3-b]pyridin-6-yl)-2-(piperazin-1-ylmethyl)cyclopropane-1-carboxamide (Compound 171 and Compound 172)

### Step 1: Synthesis of Methyl cis-2-((3-(2,6-dimethoxyphenyl)-1-((2-(trimethylsilyl)ethoxy)methyl)-1H-pyrrolo[2,3-b]pyridin-6-yl)carbamoyl)cyclopropane-1-carboxylate

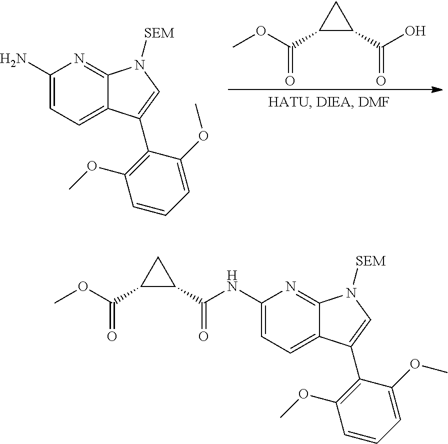

To a solution of 3-(2,6-dimethoxyphenyl)-1-((2-(trimethylsilyl)ethoxy)methyl)-1H-pyrrolo[2,3-b]pyridin-6-amine (500.0 mg, 1.25 mmol) in DMF (10.0 mL) was added cis-2-(methoxycarbonyl)cyclopropane-1-carboxylic acid (180.4 mg, 1.25 mmol), DIEA (808.6 mg, 6.26 mmol) and HATU (570.9 mg, 1.50 mmol) at 0° C. under N₂. The resulting mixture was stirred at room temperature for 2 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (62/38, v/v) to afford methyl cis-2-((3-(2,6-dimethoxyphenyl)-1-((2-(trimethylsilyl)ethoxy)methyl)-1H-pyrrolo[2,3-b]pyridin-6-yl)carbamoyl)cyclopropane-1-carboxylate (640.0 mg, 97%) as a yellow oil. LCMS (ESI, m/z): [M+H]⁺=526.2.

### Step 2: Synthesis of Cis-N-(3-(2,6-dimethoxyphenyl)-1-((2-(trimethylsilyl)ethoxy)methyl)-1H-pyrrolo[2,3-b]pyridin-6-yl)-2-(hydroxymethyl)cyclopropane-1-carboxamide

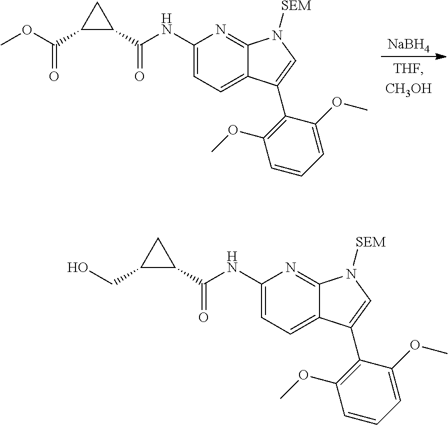

To a solution of methyl cis-2-((3-(2,6-dimethoxyphenyl)-1-((2-(trimethylsilyl)ethoxy)methyl)-1H-pyrrolo[2,3-b]pyridin-6-yl)carbamoyl)cyclopropane-1-carboxylate (640.0 mg, 1.21 mmol) in THF/CH₃OH (10.0 mL/10.0 mL) was added NaBH₄ (460.6 mg, 12.17 mmol) at room temperature. The resulting mixture was stirred at room temperature for 16 h. After the reaction was completed, the resulting mixture was quenched with H₂O at 0° C. and extracted with ethyl acetate. The combined organic layer was washed with brine, dried over anhydrous sodium sulfate and filtered. The filtrate was concentrated under reduced pressure to afford cis-N-(3-(2,6-dimethoxyphenyl)-1-((2-(trimethylsilyl)ethoxy)methyl)-1H-pyrrolo[2,3-b]pyridin-6-yl)-2-(hydroxymethyl)cyclopropane-1-carboxamide (510.0 mg, crude) as a light yellow oil. LCMS (ESI, m/z): [M+H]⁺=498.2.

### Step 3: Synthesis of Cis-N-(3-(2,6-dimethoxyphenyl)-1-((2-(trimethylsilyl)ethoxy)methyl)-1H-pyrrolo[2,3-b]pyridin-6-yl)-2-formylcyclopropane-1-carboxamide

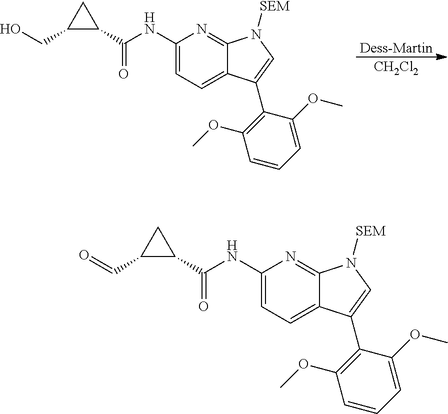 [SMILES: COc1cccc(OC)c1-c1cn(COCC[Si](C)(C)C)c2nc(NC(=O)[C@H]3C[C@H]3CO)ccc12 ; "Dess-Martin" ; "CH2Cl2" ; COc1cccc(OC)c1-c1cn(COCC[Si](C)(C)C)c2nc(NC(=O)[C@H]3C[C@H]3C=O)ccc12]

To a solution of cis-N-(3-(2,6-dimethoxyphenyl)-1-((2-(trimethylsilyl)ethoxy)methyl)-1H-pyrrolo[2,3-b]pyridin-6-yl)-2-(hydroxymethyl)cyclopropane-1-carboxamide (510.0 mg, crude) in CH₂Cl₂ (20.0 mL) was added Dess-Martin (869.3 mg, 2.06 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford cis-N-(3-(2,6-dimethoxyphenyl)-1-((2-(trimethylsilyl)ethoxy)methyl)-1H-pyrrolo[2,3-b]pyridin-6-yl)-2-formylcyclopropane-1-carboxamide (840.0 mg, crude) as a brown solid. LCMS (ESI, m/z): [M+H]⁺=496.2.

### Step 4: Synthesis of tert-butyl 4-((cis-2-((3-(2,6-dimethoxyphenyl)-1-((2-(trimethylsilyl)ethoxy)methyl)-1H-pyrrolo[2,3-b]pyridin-6-yl)carbamoyl)cyclopropyl)methyl)piperazine-1-carboxylate

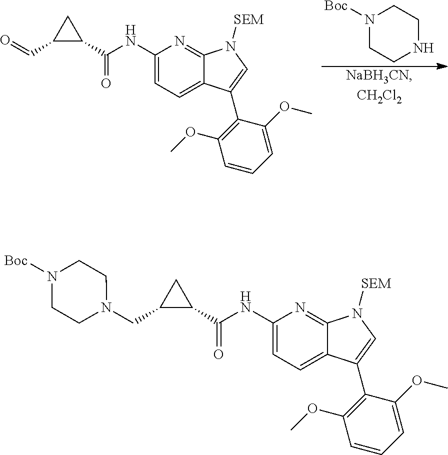

To a solution of cis-N-(3-(2,6-dimethoxyphenyl)-1-((2-(trimethylsilyl)ethoxy)methyl)-1H-pyrrolo[2,3-b]pyridin-6-yl)-2-formylcyclopropane-1-carboxamide (840.0 mg, 1.69 mmol) in CH₂Cl₂ (20.0 mL) was added tert-butyl piperazine-1-carboxylate (631.3 mg, 3.39 mmol) and NaBH₃CN (213.0 mg, 3.39 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with petroleum ether/ethyl acetate (20/80, v/v) to afford tert-butyl 4-((cis-2-((3-(2,6-dimethoxyphenyl)-1-((2-(trimethylsilyl)ethoxy)methyl)-1H-pyrrolo[2,3-b]pyridin-6-yl)carbamoyl)cyclopropyl)methyl)piperazine-1-carboxylate (660.0 mg, 58%) as a white solid. LCMS (ESI, m/z): [M+H]⁺=666.4.

### Step 5: Synthesis of (1S,2R)—N-(3-(2,6-dimethoxyphenyl)-1H-pyrrolo[2,3-b]pyridin-6-yl)-2-(piperazin-1-ylmethyl)cyclopropane-1-carboxamide and (1R,2S)—N-(3-(2,6-dimethoxyphenyl)-1H-pyrrolo[2,3-b]pyridin-6-yl)-2-(piperazin-1-ylmethyl)cyclopropane-1-carboxamide (Compound 171 and Compound 172)

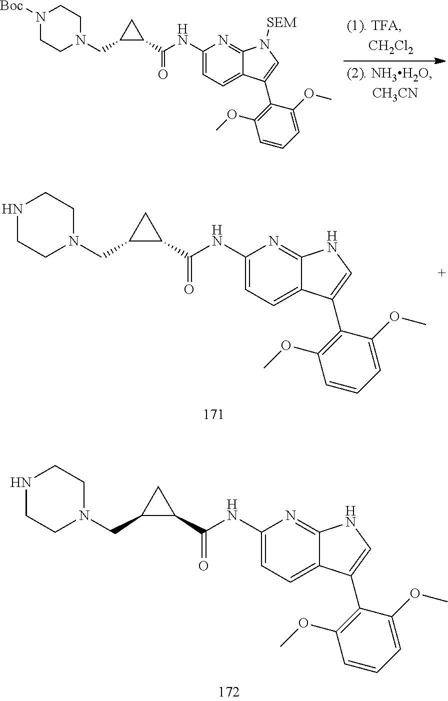 [SMILES: COc1cccc(OC)c1-c1cn(COCC[Si](C)(C)C)c2nc(NC(=O)[C@H]3C[C@H]3CN3CCN(C(=O)OC(C)(C)C)CC3)ccc12 ; COc1cccc(OC)c1-c1c[nH]c2nc(NC(=O)[C@H]3C[C@H]3CN3CCNCC3)ccc12 ; COc1cccc(OC)c1-c1c[nH]c2nc(NC(=O)[C@@H]3C[C@@H]3CN3CCNCC3)ccc12]

To a solution of tert-butyl 4-((cis-2-((3-(2,6-dimethoxyphenyl)-1-((2-(trimethylsilyl)ethoxy)methyl)-1H-pyrrolo[2,3-b]pyridin-6-yl)carbamoyl)cyclopropyl)methyl)piperazine-1-carboxylate (75.0 mg, 0.16 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in ACN (5.0 mL) was added NH₃·H₂O (5.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was separated by Prep-Chiral-HPLC with the following conditions (Column: Lux 5 um Cellulose-2, 2.12×25 cm, 5 m; Mobile Phase A: Hex (0.5% 2M NH₃-MeOH)—HPLC, Mobile Phase B: EtOH—HPLC; Flow rate: 20 mL/min; Gradient: 30% B to 30% B in 24 min; 254 nm) to afford N-(3-(2,6-dimethoxyphenyl)-1H-pyrrolo[2,3-b]pyridin-6-yl)-2-(piperazin-1-ylmethyl)cyclopropane-1-carboxamide Enantiomer 1 (5.1 mg, 2%) as a white solid and N-(3-(2,6-dimethoxyphenyl)-1H-pyrrolo[2,3-b]pyridin-6-yl)-2-(piperazin-1-ylmethyl)cyclopropane-1-carboxamide Enantiomer 2 (3.2 mg, 1%) as a white solid. The absolute stereochemistry of Enantiomers 1 and 2 was not assigned. The two enantiomeric structures that could be obtained from chiral separation of the enantiomeric mixture as described above are shown as Compounds 171 and 172 in Table 1.

N-(3-(2,6-dimethoxyphenyl)-1H-pyrrolo[2,3-b]pyridin-6-yl)-2-(piperazin-1-ylmethyl)cyclopropane-1-carboxamide Enantiomer 1: Retention Time 1: 15.594 min; LCMS (ESI, m/z): [M+H]⁺=436.3. ¹H NMR (400 MHz, DMSO-d₆): δ 11.39 (s, 1H), 10.46 (s, 1H), 7.79 (d, J=8.4 Hz, 1H), 7.52 (d, J=8.8 Hz, 1H), 7.29-7.25 (m, 2H), 6.75 (d, J=8.4 Hz, 2H), 3.69 (s, 6H), 2.67-2.62 (m, 3H), 2.47-2.42 (m, 1H), 2.38-2.21 (m, 4H), 2.10-2.06 (m, 1H), 1.36-1.30 (m, 1H), 1.04-0.91 (m, 1H), 0.89-0.82 (m, 1H).

N-(3-(2,6-dimethoxyphenyl)-1H-pyrrolo[2,3-b]pyridin-6-yl)-2-(piperazin-1-ylmethyl)cyclopropane-1-carboxamide Enantiomer 2: Retention Time 2: 21.022 min; LCMS (ESI, m/z): [M+H]⁺=436.3. ¹H NMR (400 MHz, DMSO-d₆): δ 11.39 (s, 1H), 10.46 (s, 1H), 7.79 (d, J=8.4 Hz, 1H), 7.52 (d, J=8.8 Hz, 1H), 7.27-7.25 (m, 2H), 6.75 (d, J=8.4 Hz, 2H), 3.69 (s, 6H), 2.67-2.61 (m, 3H), 2.47-2.30 (m, 4H), 2.10-2.06 (m, 1H), 1.35-1.32 (m, 1H), 0.98-0.91 (m, 1H), 0.89-0.82 (m, 1H)

### Example S166: Synthesis of 1-[3-(4-cyclopropoxy-2-methoxypyridin-3-yl)-1H-pyrrolo[2,3-b]pyridin-6-yl]-3-[3-(dimethylamino)propyl]urea (Compound 173)

### Step 1: Synthesis of 3-(6-chloro-1-{[2-(trimethylsilyl)ethoxy]methyl}pyrrolo[2,3-b]pyridin-3-yl)-4-cyclopropoxy-2-methoxypyridine

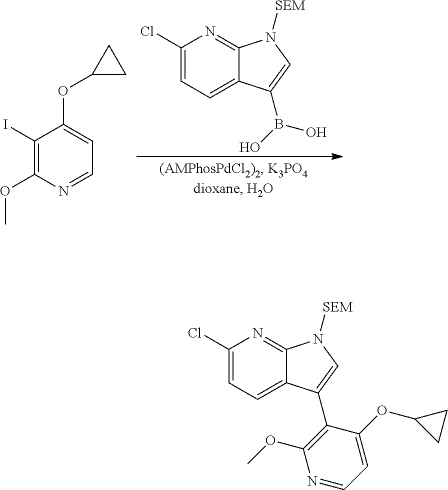

To a solution of 4-cyclopropoxy-3-iodo-2-methoxypyridine (500.0 mg, 1.72 mmol) in 1, 4-dioxane (20.0 mL)/H₂O (5.0 mL) was added K₃PO₄ (1093.8 mg, 5.15 mmol), 6-chloro-1-{[2-(trimethylsilyl)ethoxy]methyl}pyrrolo[2,3-b]pyridin-3-ylboronic acid (561.1 mg, 1.72 mmol) and (AMPhosPdCl₂)₂ (243.3 mg, 0.34 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (5/1, v/v) to afford 3-(6-chloro-1-{[2-(trimethylsilyl)ethoxy]methyl}pyrrolo[2,3-b]pyridin-3-yl)-4-cyclopropoxy-2-methoxypyridine (650.0 mg, 84%) as a yellow oil. LCMS (ESI, m/z): [M+H]⁺=446.2.

### Step 2: Synthesis of 3-(4-cyclopropoxy-2-methoxypyridin-3-yl)-1-{[2-(trimethylsilyl)ethoxy]methyl}pyrrolo[2,3-b]pyridin-6-amine

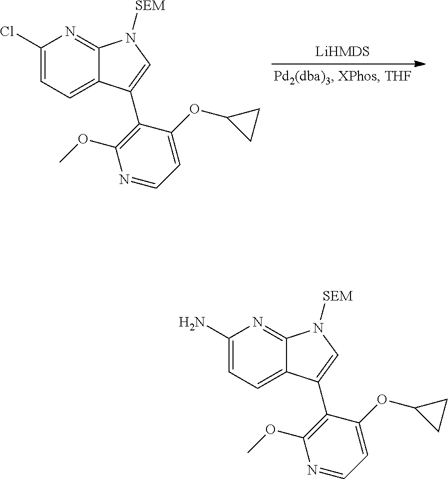 [SMILES: COc1nccc(OC2CC2)c1-c1cn(COCC[Si](C)(C)C)c2nc(Cl)ccc12 ; COc1nccc(OC2CC2)c1-c1cn(COCC[Si](C)(C)C)c2nc(N)ccc12]

To a solution of 3-(6-chloro-1-{[2-(trimethylsilyl)ethoxy]methyl}pyrrolo[2,3-b]pyridin-3-yl)-4-cyclopropoxy-2-methoxypyridine (524.0 mg, 1.2 mmol) in THF (20.0 mL) was added Pd₂(dba)₃ (107.6 mg, 0.12 mmol), LiHMDS (3.5 mL, 1.0 mol/L) and XPhos (112.0 mg, 0.24 mmol) at room temperature under N₂. The resulting mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 3-(4-cyclopropoxy-2-methoxypyridin-3-yl)-1-{[2-(trimethylsilyl)ethoxy]methyl}pyrrolo[2,3-b]pyridin-6-amine (440.0 mg, 87%) as a yellow solid. LCMS (ESI, m/z): [M+H]⁺=427.2.

### Step 3: Synthesis of 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)propyl]urea

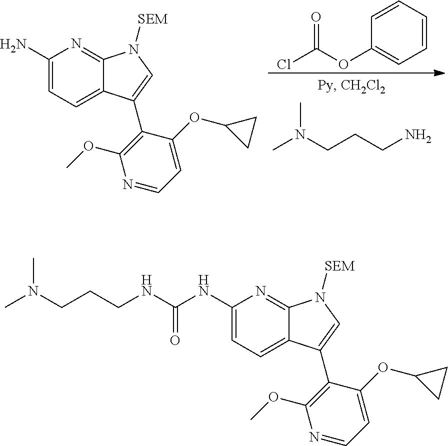

To a solution of 3-(4-cyclopropoxy-2-methoxypyridin-3-yl)-1-{[2-(trimethylsilyl)ethoxy]methyl}pyrrolo[2,3-b]pyridin-6-amine (200.0 mg, 0.47 mmol) in CH₂Cl₂ (15.0 mL) was added Pyridine (148.3 mg, 1.88 mmol) and phenyl chloroformate (146.8 mg, 0.94 mmol) at 0° C. The resulting mixture was stirred at 0° C. for 1 h. The resulting mixture was concentrated under reduced pressure. To the above residue was added N¹, N¹-dimethylpropane-1,3-diamine (335.3 mg, 3.28 mmol) and Pyridine (10.0 mL) at room temperature. The resulting mixture was stirred at 60 C for 3 h. After the reaction was completed, the resulting mixture was concentrated under reduced pressure. The residue was purified by flash column chromatography with CH₂Cl₂/CH₃OH (20/1, v/v) to afford 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)propyl]urea (140.0 mg, 53%) as a yellow solid. LCMS (ESI, m/z): [M+H]⁺=555.3.

### Step 4: Synthesis of 1-[3-(4-cyclopropoxy-2-methoxypyridin-3-yl)-1H-pyrrolo[2,3-b]pyridin-6-yl]-3-[3-(dimethylamino)propyl]urea (Compound 173)

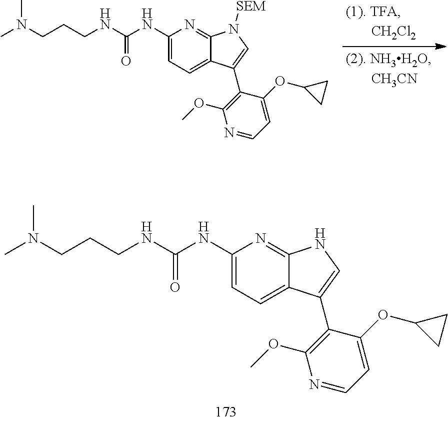 [SMILES: COc1nccc(OC2CC2)c1-c1cn(COCC[Si](C)(C)C)c2nc(NC(=O)NCCCN(C)C)ccc12 ; COc1nccc(OC2CC2)c1-c1c[nH]c2nc(NC(=O)NCCCN(C)C)ccc12]

To a solution of 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)propyl]urea (120.0 mg, 0.22 mmol) in CH₂Cl₂ (8.0 mL) was added TFA (8.0 mL) at room temperature. The resulting mixture was stirred at room temperature for 3 h. The resulting mixture was concentrated under reduced pressure. To the above residue was added NH₃·H₂O (8.0 mL) and CH₃CN (8.0 mL) at room temperature. The resulting mixture was stirred at room temperature for 3 h. After the reaction was completed, the resulting mixture was concentrated under reduced pressure. The residue was purified by Prep-HPLC with the following conditions (Column: XBridge Prep OBD Cis Column, 30×150 mm, 5 m; Mobile Phase A: Water (10 mmol/L NH₄HCO₃), Mobile Phase B: ACN; Flow rate: 60.0 mL/min; Gradient: 18% B to 35% B in 8 min; Wave Length: 254 nm) to afford 1-[3-(4-cyclopropoxy-2-methoxypyridin-3-yl)-1H-pyrrolo[2,3-b]pyridin-6-yl]-3-[3-(dimethylamino)propyl]urea (Compound 173) (37.4 mg, 39%) as a white solid. LCMS (ESI, m/z): [M+H]⁺=425.4. ¹H NMR (400 MHz, DMSO-d₆): δ 11.43 (s, 1H), 9.11 (s, 1H), 8.53 (s, 1H); 8.06 (d, J=6.0 Hz, 1H), 7.49 (d, J=8.4 Hz, 1H), 7.23 (d, J=2.4 Hz, 1H), 7.13 (d, J=5.6 Hz, 1H), 6.95 (d, J=8.4 Hz, 1H), 3.94-3.90 (m, 1H), 3.81 (s, 3H), 3.27-3.22 (m, 2H), 2.29-2.26 (m, 2H), 2.15 (s, 6H), 1.69-1.62 (m, 2H), 0.81-0.78 (m, 2H), 0.66-0.61 (m, 2H).

### Example S167: Synthesis of 1-[3-(4-cyclopropoxy-6-methoxypyrimidin-5-yl)-1H-pyrrolo[2,3-b]pyridin-6-yl]-3-[3-(dimethylamino)propyl]urea (Compound 174)

### Step 1: Synthesis of 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)propyl]urea

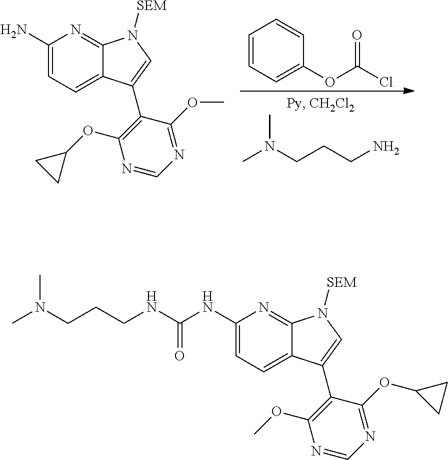

To a solution of 3-(4-cyclopropoxy-6-methoxypyrimidin-5-yl)-1-{[2-(trimethylsilyl)ethoxy]methyl}pyrrolo[2,3-b]pyridin-6-amine (600.0 mg, 1.40 mmol) in CH₂Cl₂ (6.0 mL) was added phenyl chloroformate (263.7 mg, 1.68 mmol) and pyridine (444.0 mg, 5.61 mmol) at room temperature. The resulting mixture was stirred at room temperature for 1 h. The resulting mixture was concentrated under reduced pressure. To the residue in pyridine (6.0 mL) was added N¹,N¹-dimethylpropane-1,3-diamine (430.2 mg, 4.21 mmol) at room temperature. The resulting mixture was stirred at 60° C. for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash chromatography with CH₂Cl₂/CH₃OH (90/10, v/v) to afford 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)propyl]urea (390.0 mg, 50%) as a yellow solid. LCMS (ESI, m/z): [M+H]⁺=556.3.

### Step 2: Synthesis of 1-[3-(4-cyclopropoxy-6-methoxypyrimidin-5-yl)-1H-pyrrolo[2,3-b]pyridin-6-yl]-3-[3-(dimethylamino)propyl]urea (Compound 174)

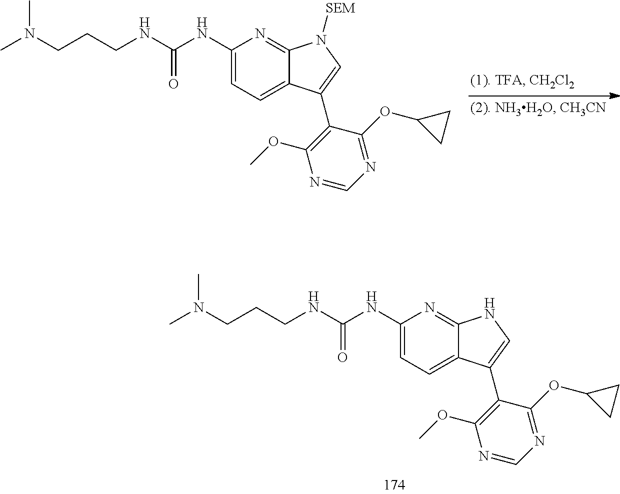 [SMILES: COc1ncnc(OC2CC2)c1-c1cn(COCC[Si](C)(C)C)c2nc(NC(=O)NCCCN(C)C)ccc12 ; COc1ncnc(OC2CC2)c1-c1c[nH]c2nc(NC(=O)NCCCN(C)C)ccc12]

To a solution of 1-[3-(4-cyclopropoxy-6-methoxypyrimidin-5-yl)-1-{[2-(trimethylsilyl)ethoxy]methyl}pyrrolo[2,3-b]pyridin-6-yl]-3-[3-(dimethylamino)propyl]urea (200.0 mg, 0.36 mmol) in CH₂Cl₂ (4.0 mL) was added TFA (4.0 mL) at room temperature. The resulting mixture was stirred at room temperature for 2 h. The resulting mixture was concentrated under reduced pressure. To the residue in CH₃CN (4.0 mL) was added NH₃·H₂O (4.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 18% B to 38% B in 8 min, 38% B; Wave Length: 254 nm) to afford 1-[3-(4-cyclopropoxy-6-methoxypyrimidin-5-yl)-1H-pyrrolo[2,3-b]pyridin-6-yl]-3-[3-(dimethylamino)propyl]urea (Compound 174) (56.7 mg, 36%) as a white solid. LCMS (ESI, m/z): [M+H]⁺=426.2. ¹H NMR (400 MHz, DMSO-d₆): δ 11.56 (s, 1H), 9.15 (s, 1H), 8.48 (s, 2H), 7.58 (d, J=8.4 Hz, 1H), 7.33 (s, 1H), 6.98 (d, J=8.8 Hz, 1H), 4.38-4.33 (m, 1H), 3.91 (s, 3H), 3.26-3.21 (m, 2H), 2.29-2.25 (m, 2H), 2.14 (s, 6H), 1.69-1.62 (m, 2H), 0.77-0.74 (m, 2H), 0.69-0.59 (m, 2H).

### Example S168: Synthesis of (1S,2S)—N-[3-(3,5-dimethoxypyridazin-4-yl)-1H-pyrrolo[2,3-b]pyridin-6-yl]-2-fluorocyclopropane-1-carboxamide (Compound 175)

### Step 1: Synthesis of 4-bromo-5-methoxy-2H-pyridazin-3-one

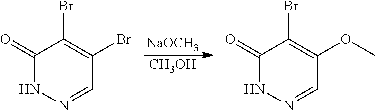

To a solution of 4,5-dibromo-2H-pyridazin-3-one (5.0 g, 19.69 mmol) in CH₃OH (80.0 mL) was added NaOMe (3.2 g, 59.08 mmol) at room temperature under N₂. The resulting mixture was stirred at 80° C. for 16 h under N₂. After the reaction was completed, the reaction solution was concentrated under reduced pressure. The residue was purified by reverse phase flash chromatography with ACN/H₂O (1/10, v/v) to afford 4-bromo-5-methoxy-2H-pyridazin-3-one (150.0 mg, 4%) as a white solid. LCMS (ESI, m/z): [M+H]⁺=205.0.

### Step 2: Synthesis of 4-bromo-3,5-dimethoxypyridazine

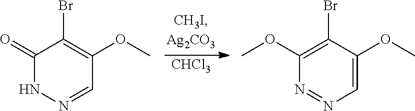

To a solution of 4-bromo-5-methoxy-2H-pyridazin-3-one (2.0 g, 9.76 mmol) in CHCl₃ (30.0 mL) was added Ag₂CO₃ (10.8 g, 39.02 mmol) and CH₃I (11.1 g, 78.04 mmol) at room temperature. The resulting mixture was stirred at room temperature for 16 h. After the reaction was completed, the resulting mixture was filtered. The filtrate was concentrated under reduced pressure. The residue was purified by reverse phase flash chromatography with ACN/H₂O (1/2, v/v) to afford 4-bromo-3,5-dimethoxypyridazine (100.0 mg, 4%) as a white solid. LCMS (ESI, m/z): [M+H]⁺=219.0.

### Step 3: Synthesis of 4-(6-chloro-1-{[2-(trimethylsilyl)ethoxy]methyl}pyrrolo[2,3-b]pyridin-3-yl)-3,5-dimethoxypyridazine

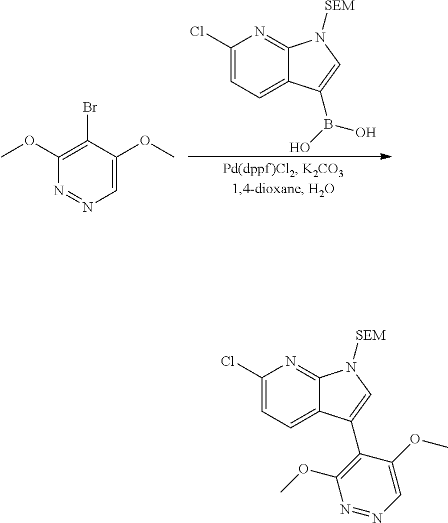

To a solution of 4-bromo-3,5-dimethoxypyridazine (130.0 mg, 0.59 mmol) in dioxane/H₂O (10.0 mL/2.0 mL) was added 6-chloro-1-{[2-(trimethylsilyl)ethoxy]methyl}pyrrolo[2,3-b]pyridin-3-ylboronic acid (193.9 mg, 0.59 mmol), K₂CO₃ (246.1 mg, 1.78 mmol) and Pd(dppf)Cl₂ (43.4 mg, 0.06 mmol) at room temperature under N₂. The resulting mixture was stirred at 80° C. for 16 h under N₂. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 4-(6-chloro-1-{[2-(trimethylsilyl)ethoxy]methyl}pyrrolo[2,3-b]pyridin-3-yl)-3,5-dimethoxypyridazine (70.0 mg, 28%) as a yellow oil. LCMS (ESI, m/z): [M+H]⁺=421.1.

### Step 4: Synthesis of (1S,2S)—N-[3-(3,5-dimethoxypyridazin-4-yl)-1-{[2-(trimethylsilyl)ethoxy]methyl}pyrrolo[2,3-b]pyridin-6-yl]-2-fluorocyclopropane-1-carboxamide

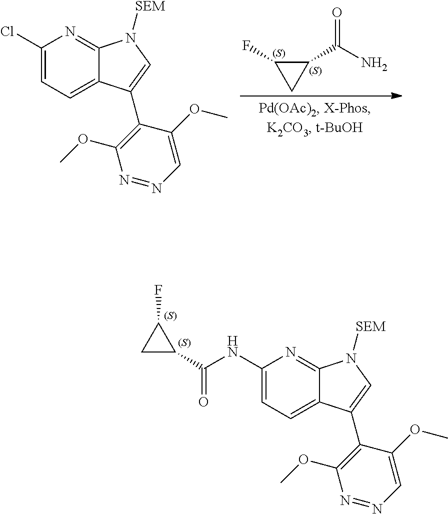

To a solution of 4-(6-chloro-1-{[2-(trimethylsilyl)ethoxy]methyl}pyrrolo[2,3-b]pyridin-3-yl)-3,5-dimethoxypyridazine (80.0 mg, 0.19 mmol) in t-BuOH (6.0 mL) was added (1S,2S)-2-fluorocyclopropane-1-carboxamide (97.9 mg, 0.95 mmol), X-Phos (18.1 mg, 0.04 mmol), K₂CO₃ (78.8 mg, 0.57 mmol) and Pd(OAc)₂ (4.3 mg, 0.02 mmol) at room temperature under N₂. The resulting mixture was stirred at 100° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (1/2, v/v) to afford (1S,2S)—N-[3-(3,5-dimethoxypyridazin-4-yl)-1-{[2-(trimethylsilyl)ethoxy]methyl}pyrrolo[2,3-b]pyridin-6-yl]-2-fluorocyclopropane-1-carboxamide (30.0 mg, 32%) as a yellow oil. LCMS (ESI, m/z): [M+H]⁺=488.2.

### Step 5: Synthesis of (1S,2S)—N-[3-(3,5-dimethoxypyridazin-4-yl)-1H-pyrrolo[2,3-b]pyridin-6-yl]-2-fluorocyclopropane-1-carboxamide (Compound 175)

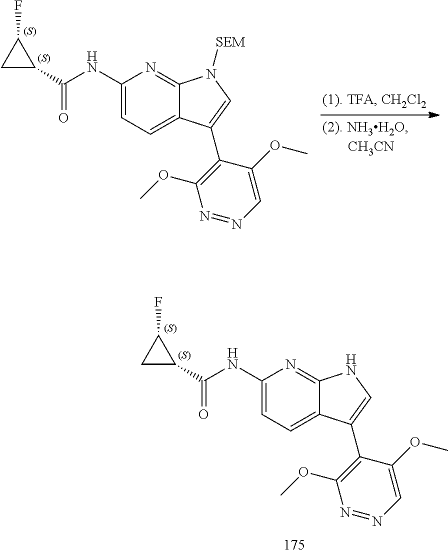

To a solution of (1S,2S)—N-[3-(3,5-dimethoxypyridazin-4-yl)-1-{[2-(trimethylsilyl)ethoxy]methyl}pyrrolo[2,3-b]pyridin-6-yl]-2-fluorocyclopropane-1-carboxamide (50.0 mg, 0.12 mmol) in CH₂Cl₂ (2.0 mL) was added TFA (2.0 mL) at room temperature. The resulting mixture was stirred at room temperature for 4 h. The mixture was concentrated under reduced pressure. To the residue in CH₃CN (2.0 mL) was added NH₃·H₂O (2.0 mL) at room temperature. The resulting mixture was stirred at room temperature for 16 h. After the reaction was completed, the resulting mixture was concentrated under reduced pressure. The residue was purified by Prep-HPLC with the following conditions Column (YMC-Actus Triart C18 ExRS, 30×150 mm, 5 m; Mobile Phase A: Water (10 mmol/L NH₄HCO₃), Mobile Phase B: ACN; Flow rate: 60 mL/min; Gradient: 15% B to 40% B in 8 min; 254 nm) to afford (1S,2S)—N-[3-(3,5-dimethoxypyridazin-4-yl)-1H-pyrrolo[2,3-b]pyridin-6-yl]-2-fluorocyclopropane-1-carboxamide (Compound 175) (6.6 mg, 17%) as a white solid. LCMS (ESI, m/z): [M+H]⁺=358.2. ¹H NMR (400 MHz, DMSO-d₆): δ 11.84 (s, 1H), 10.64 (s, 1H), 9.01 (s, 1H), 7.90 (d, J=8.8 Hz, 1H), 7.77 (d, J=8.8 Hz, 1H), 7.64 (s, 1H), 5.01-4.84 (m, 1H), 4.04 (s, 3H), 3.99 (s, 3H), 2.26-2.22 (m, 1H), 1.70-1.63 (m, 1H), 1.19-1.13 (m, 1H).

### Example S169: Synthesis of (1S,2S)-2-fluoro-N-(3-(4-fluoro-5,7-dimethoxybenzo[d]thiazol-6-yl)-1H-pyrrolo[2,3-b]pyridin-6-yl)cyclopropane-1-carboxamide (Compound 176)

### Step 1: Synthesis of tert-butyl N-(3,5-dimethoxyphenyl)carbamate

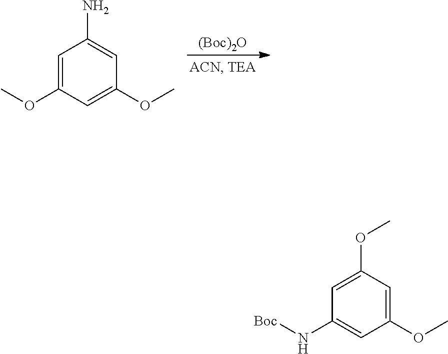

To a solution of 3,5-dimethoxyaniline (10.0 g, 6.52 mmol) in ACN (10.0 mL) was added Boc₂O (21.3 g, 97.93 mmol) and TEA (19.8 g, 195.84 mmol) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-(3,5-dimethoxyphenyl)carbamate (5.0 g, 30%) as a white solid. LCMS (ESI, m/z): [M+H]⁺=254.1.

### Step 2: Synthesis of tert-butyl N-(2-fluoro-3,5-dimethoxyphenyl)carbamate

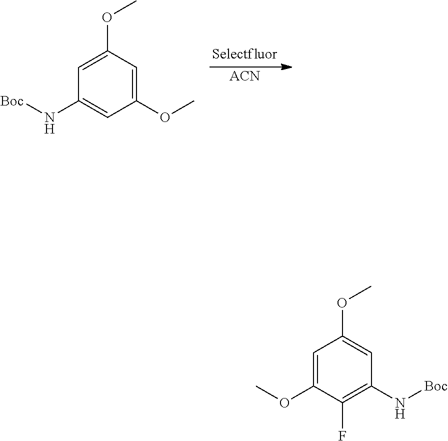

To a solution of Selectfluor (5.0 g, 15.72 mmol) in ACN (50.0 mL) was added tert-butyl N-(3,5-dimethoxyphenyl)carbamate (4.0 g, 15.72 mmol) at 0° C. The resulting mixture was stirred at 0° C. for 2 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford tert-butyl N-(2-fluoro-3,5-dimethoxyphenyl)carbamate (1.2 g, 28%) as a white solid. LCMS (ESI, m/z): [M+H]⁺=272.1.

### Step 3: Synthesis of 2-fluoro-3,5-dimethoxyaniline

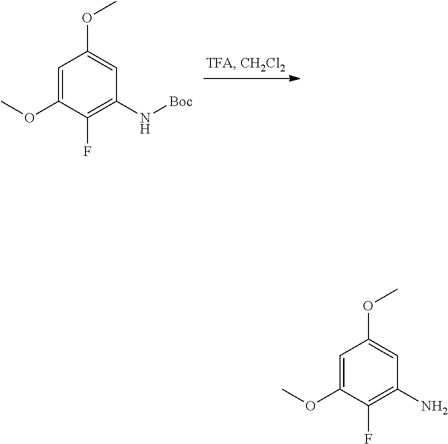 [SMILES: COc1cc(NC(=O)OC(C)(C)C)c(F)c(OC)c1 ; COc1cc(N)c(F)c(OC)c1]

To a solution of tert-butyl N-(2-fluoro-3,5-dimethoxyphenyl)carbamate (1.0 g, 3.66 mmol) in DCM (5.0 mL) was added TFA (5.0 mL) at room temperature. The resulting mixture was stirred at room temperature for 2 h. After the reaction was completed, the resulting mixture was diluted with H₂O. The pH value of the mixture was adjusted to 7 with aq. NaHCO₃. The mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 2-fluoro-3,5-dimethoxyaniline (500.0 mg, 79%) as a red solid. LCMS (ESI, m/z): [M+H]⁺=172.1.

### Step 4: Synthesis of 1-benzoyl-3-(2-fluoro-3,5-dimethoxyphenyl)thiourea

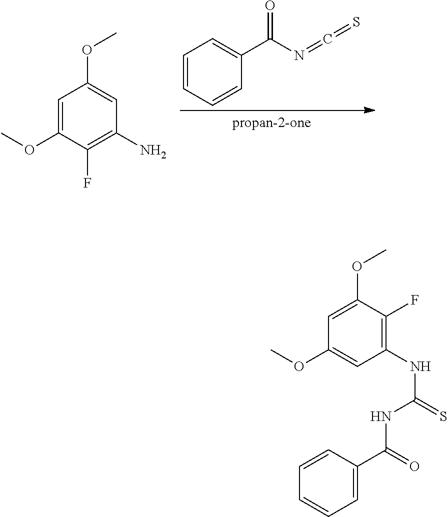 [SMILES: COc1cc(N)c(F)c(OC)c1 ; O=C(N=C=S)c1ccccc1 ; COc1cc(NC(=S)NC(=O)c2ccccc2)c(F)c(OC)c1]

To a solution of 2-fluoro-3,5-dimethoxyaniline (500.0 mg, 2.91 mmol) in propan-2-one (5.0 mL) was added benzoyl isothiocyanate (441.6 mg, 2.91 mmol) at room temperature. The resulting mixture was stirred at 60° C. for 12 h. After the reaction was completed, the resulting mixture was filtered. The solid was collected and dried to afford 1-benzoyl-3-(2-fluoro-3,5-dimethoxyphenyl)thiourea (500.0 mg, crude) as a red oil. LCMS (ESI, m/z): [M+H]⁺=335.1.

### Step 5: Synthesis of 2-fluoro-3,5-dimethoxyphenylthiourea

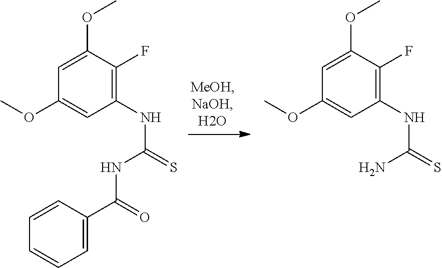 [SMILES: COc1cc(NC(=S)NC(=O)c2ccccc2)c(F)c(OC)c1 ; COc1cc(NC(N)=S)c(F)c(OC)c1]

To a solution of 1-benzoyl-3-(2-fluoro-3,5-dimethoxyphenyl)thiourea (500.0 mg, crude) in MeOH (5.0 mL) and H₂O (5.0 mL) was added NaOH (39.6 mg, 1.65 mmol). The resulting mixture was stirred at 80° C. for 12 h. After the reaction was completed, the resulting mixture was filtered. The solid was washed with CH₃OH and then dried to afford 2-fluoro-3,5-dimethoxyphenylthiourea (300.0 mg, crude) as a white solid. LCMS (ESI, m/z): [M+H]⁺=231.1.

### Step 6: Synthesis of 4-fluoro-5,7-dimethoxy-1,3-benzothiazol-2-amine

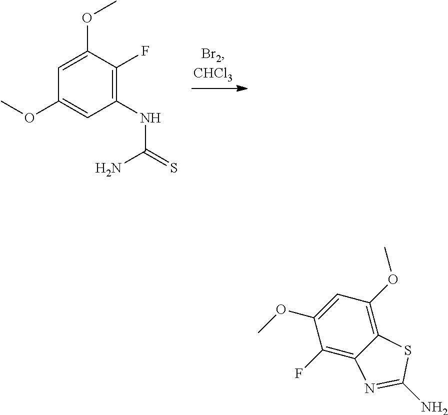

To a solution of 2-fluoro-3,5-dimethoxyphenylthiourea (300.0 mg, 1.34 mmol) in CHCl₃ (5.0 mL) was added Br₂ (417.4 mg, 1.5 mmol). The resulting mixture was stirred at 60° C. for 2 h. After the reaction was completed, the resulting mixture was filtered. The solid was collected and dried to afford 4-fluoro-5,7-dimethoxy-1,3-benzothiazol-2-amine (250.0 mg, crude) as a white solid. LCMS (ESI, m/z): [M+H]⁺=229.0.

### Step 7: Synthesis of 6-bromo-4-fluoro-5,7-dimethoxy-1,3-benzothiazol-2-amine

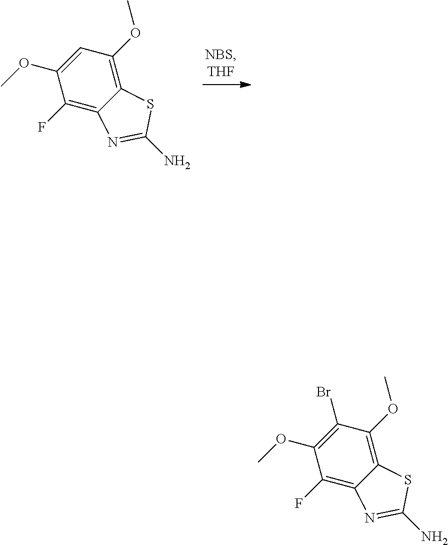 [SMILES: COc1cc(OC)c2sc(N)nc2c1F ; COc1c(Br)c(OC)c2sc(N)nc2c1F]

To a solution of 4-fluoro-5,7-dimethoxy-1,3-benzothiazol-2-amine (250.0 mg, 1.10 mmol) in THE (5.0 mL) was added NBS (300.0 mg, 1.64 mmol). The resulting mixture was stirred at 60° C. for 2 h. After the reaction was completed, the resulting mixture was filtered. The solid was collected and dried to afford 6-bromo-4-fluoro-5,7-dimethoxy-1,3-benzothiazol-2-amine (250.0 mg, crude) as an off-white solid. LCMS (ESI, m/z): [M+H]⁺=306.9.

### Step 8: Synthesis of 6-bromo-4-fluoro-5,7-dimethoxy-1,3-benzothiazole

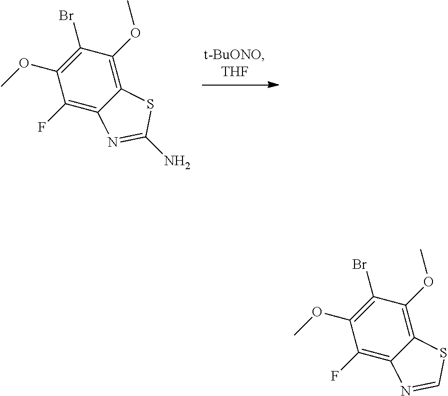 [SMILES: COc1c(Br)c(OC)c2sc(N)nc2c1F ; COc1c(Br)c(OC)c2scnc2c1F]

To a solution of 6-bromo-4-fluoro-5,7-dimethoxy-1,3-benzothiazol-2-amine (250.0 mg, crude) in THF (5.0 mL) was added tert-butyl nitrite (125.9 mg, 1.36 mmol) at room temperature. The resulting mixture was stirred at room temperature for 2 h. After the reaction was completed, the resulting mixture was diluted with H₂O and extracted with dichloromethane. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-bromo-4-fluoro-5,7-dimethoxy-1,3-benzothiazole (200.0 mg, 84%) as a white solid. LCMS (ESI, m/z): [M+H]⁺=291.9.

### Step 9: Synthesis of 6-(6-chloro-1-{[2-(trimethylsilyl)ethoxy]methyl}pyrrolo[2,3-b]pyridin-3-yl)-4-fluoro-5,7-dimethoxy-1,3-benzothiazole

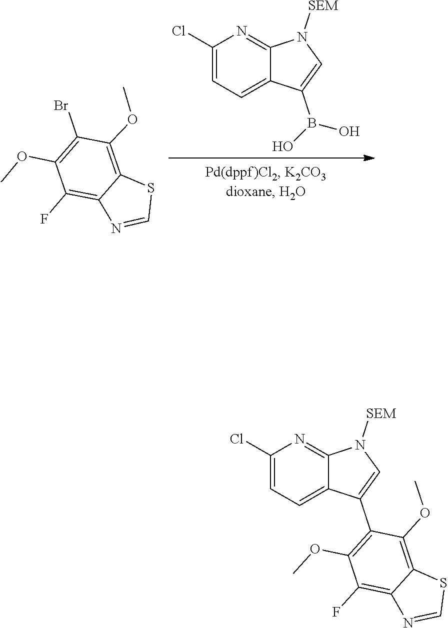 [SMILES: COc1c(Br)c(OC)c2scnc2c1F ; C[Si](C)(C)CCOCn1cc(B(O)O)c2ccc(Cl)nc21 ; COc1c(-c2cn(COCC[Si](C)(C)C)c3nc(Cl)ccc23)c(OC)c2scnc2c1F]

To a solution of 6-bromo-4-fluoro-5,7-dimethoxy-1,3-benzothiazole (200.0 mg, 0.69 mmol) in dioxane (5.0 mL) and H₂O (0.5 mL) was added 6-chloro-1-{[2-(trimethylsilyl)ethoxy]methyl}pyrrolo[2,3-b]pyridin-3-ylboronic acid (268.4 mg, 0.82 mmol), Pd(dppf)Cl₂ (50.2 mg, 0.07 mmol) and K₂CO₃ (283.9 mg, 2.07 mmol) under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 6-(6-chloro-1-{[2-(trimethylsilyl)ethoxy]methyl}pyrrolo[2,3-b]pyridin-3-yl)-4-fluoro-5,7-dimethoxy-1,3-benzothiazole (200.0 mg, 59%) as a green oil. LCMS (ESI, m/z): [M+H]⁺=494.1.

### Step 10: Synthesis of (1S,2S)-2-fluoro-N-[3-(4-fluoro-5,7-dimethoxy-1,3-benzothiazol-6-yl)-1-{[2-(trimethylsilyl)ethoxy]methyl}pyrrolo[2,3-b]pyridin-6-yl]cyclopropane-1-carboxamide

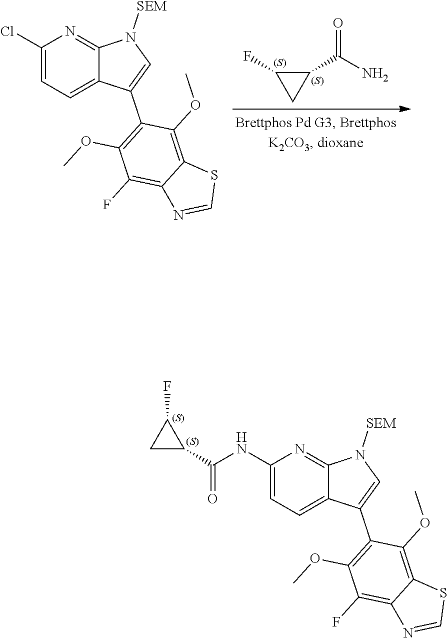

To a solution of 6-(6-chloro-1-{[2-(trimethylsilyl)ethoxy]methyl}pyrrolo[2,3-b]pyridin-3-yl)-4-fluoro-5,7-dimethoxy-1,3-benzothiazole (200.0 mg, 0.45 mmol) in dioxane (5.0 mL) was added (1S,2S)-2-fluorocyclopropane-1-carboxamide (125.2 mg, 1.25 mmol), K₂CO₃ (167.8 mg, 1.25 mmol), BrettPhos (43.4 mg, 0.08 mmol) and BrettPhos Pd G3 (36.7 mg, 0.04 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-[3-(4-fluoro-5,7-dimethoxy-1,3-benzothiazol-6-yl)-1-{[2-(trimethylsilyl)ethoxy]methyl}pyrrolo[2,3-b]pyridin-6-yl]cyclopropane-1-carboxamide (100.0 mg, 44%) as a green oil. LCMS (ESI, m/z): [M+H]⁺=561.2.

### Step 11: Synthesis of (1S,2S)-2-fluoro-N-(3-(4-fluoro-5,7-dimethoxybenzo[d]thiazol-6-yl)-1H-pyrrolo[2,3-b]pyridin-6-yl)cyclopropane-1-carboxamide (Compound 176)

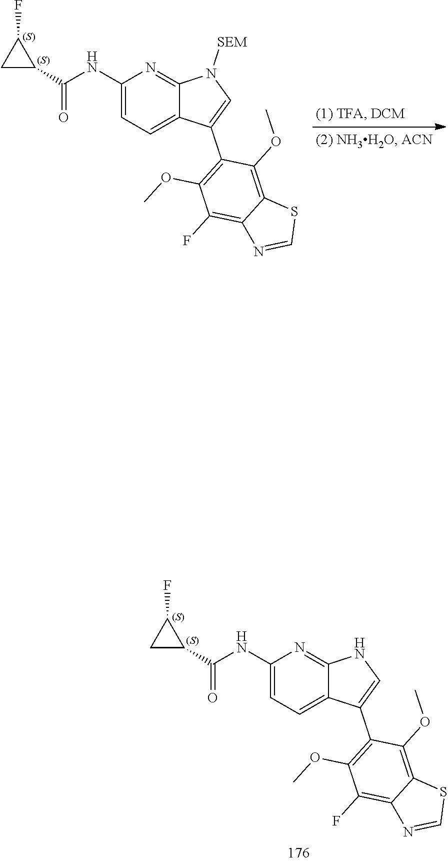

To a solution of (1S,2S)-2-fluoro-N-[3-(4-fluoro-5,7-dimethoxy-1,3-benzothiazol-6-yl)-1-{[2-(trimethylsilyl)ethoxy]methyl}pyrrolo[2,3-b]pyridin-6-yl]cyclopropane-1-carboxamide (100.0 mg, 0.18 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 49% B to 63% B in 10 min; 254 nm) to afford (1S,2S)-2-fluoro-N-(3-(4-fluoro-5,7-dimethoxybenzo[d]thiazol-6-yl)-1H-pyrrolo[2,3-b]pyridin-6-yl)cyclopropane-1-carboxamide (Compound 176) (2.3 mg, 2%) as a white solid. LCMS (ESI, m/z): [M+H]⁺=431.1. ¹H NMR (300 MHz, DMSO-d₆): δ 11.79 (s, 1H), 10.67 (s, 1H), 9.42 (s, 1H), 7.86 (d, J=8.4 Hz, 1H), 7.76 (d, J=8.7 Hz, 1H), 7.55 (d, J=2.4 Hz, 1H), 5.03-4.79 (m, 1H), 3.61 (s, 3H), 3.47 (s, 3H), 2.26-2.20 (m, 1H), 1.68-1.59 (m, 1H), 1.17-1.08 (m, 1H).

### Example S170: Synthesis of (1R,5S,6R)—N-[3-(2,6-dimethoxyphenyl)-1H-pyrrolo[2,3-b]pyridin-6-yl]-3-methyl-3-azabicyclo[3.1.0]hexane-6-carboxamide (Compound 177)

### Step 1: Synthesis of ethyl (1R,5S,6R)-3-methyl-3-azabicyclo[3.1.0]hexane-6-carboxylate

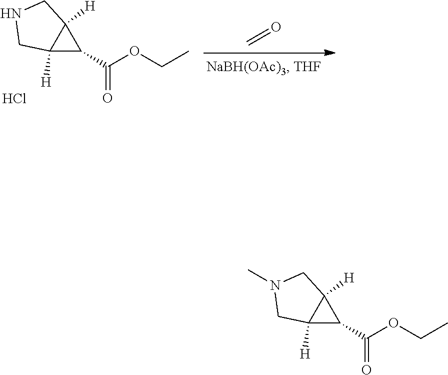

To a solution of ethyl (1R,5S,6R)-3-azabicyclo[3.1.0]hexane-6-carboxylate hydrochloride (200.0 mg, 1.04 mmol) in THE (5.0 mL) was added formaldehyde (522.2 mg, 5.22 mmol) and NaBH(OAc)₃ (442.3 mg, 2.09 mmol) at room temperature. The resulting mixture was stirred at room temperature for 2 h. After the reaction was completed, the reaction mixture was quenched with MeOH. The resulting mixture was concentrated under vacuum. The residue was purified by flash column chromatography with CH₂Cl₂/MeOH (90/10, v/v) to afford ethyl (1R,5S,6R)-3-methyl-3-azabicyclo[3.1.0]hexane-6-carboxylate (140.0 mg, 79%) as a yellow solid. LCMS (ESI, m/z): [M+H]⁺=170.1.

### Step 2: Synthesis of (1R,5S,6R)—N-[3-(2,6-dimethoxyphenyl)-1-{[2-(trimethylsilyl)ethoxy]methyl}pyrrolo[2,3-b]pyridin-6-yl]-3-methyl-3-azabicyclo[3.1.0]hexane-6-carboxamide

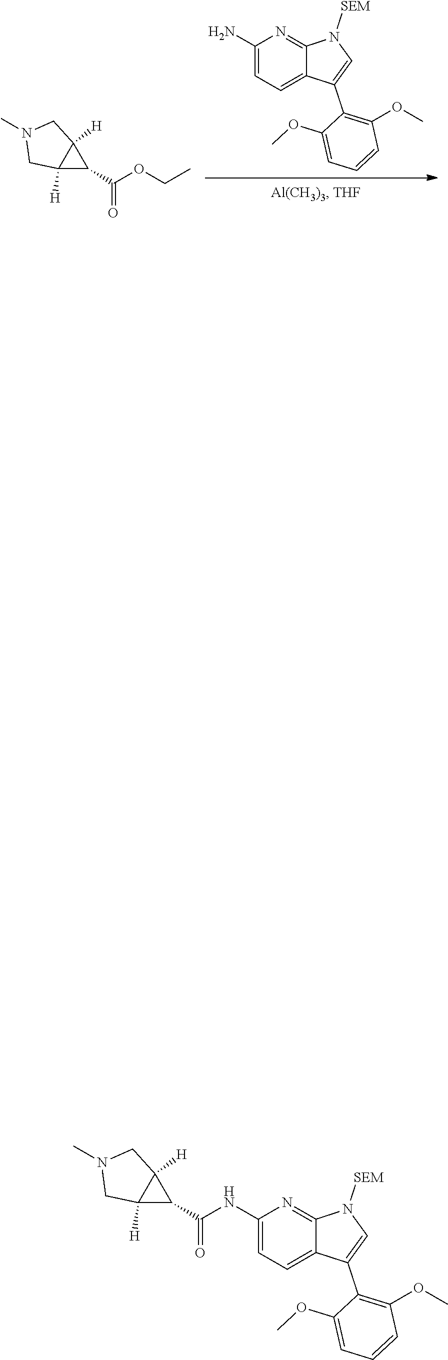

To a mixture of ethyl (1R,5S,6R)-3-methyl-3-azabicyclo[3.1.0]hexane-6-carboxylate (120.0 mg, 0.71 mmol) and 3-(2,6-dimethoxyphenyl)-1-{[2-(trimethylsilyl)ethoxy]methyl}pyrrolo[2,3-b]pyridin-6-amine (141.7 mg, 0.35 mmol) in THE (3.0 mL) was added dropwise AlMe₃ (1.8 mL, 2 mol/L) at 0° C. The mixture was stirred at 80° C. for 2 h. After the reaction was completed, the reaction mixture was quenched with water. The resulting mixture was concentrated under vacuum. The residue was purified by flash column chromatography with CH₂Cl₂/MeOH (90/10, v/v) to afford (1R,5S,6R)—N-[3-(2,6-dimethoxyphenyl)-1-{[2-(trimethylsilyl)ethoxy]methyl}pyrrolo[2,3-b]pyridin-6-yl]-3-methyl-3-azabicyclo[3.1.0]hexane-6-carboxamide (80.0 mg, 21%) as a yellow solid. LCMS (ESI, m/z): [M+H]⁺=523.3.

### Step 3: Synthesis of (1R,5S,6R)—N-[3-(2,6-dimethoxyphenyl)-1H-pyrrolo[2,3-b]pyridin-6-yl]-3-methyl-3-azabicyclo[3.1.0]hexane-6-carboxamide (Compound 177)

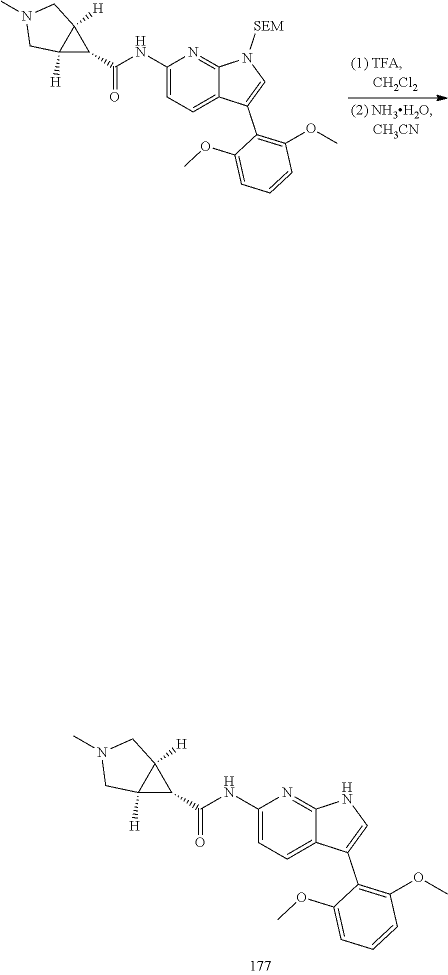

To a solution of (1R,5S,6R)—N-[3-(2,6-dimethoxyphenyl)-1-{[2-(trimethylsilyl)ethoxy]methyl}pyrrolo[2,3-b]pyridin-6-yl]-3-methyl-3-azabicyclo[3.1.0]hexane-6-carboxamide (60.0 mg, 0.12 mmol) in CH₂Cl₂ (2.0 mL) was added TFA (2.0 mL). The mixture was stirred at room temperature for 1 h. The mixture was concentrated under vacuum. To the above residue were added ACN (2.0 mL) and NH₃·H₂O (2.0 mL). The resulting mixture was stirred at room temperature for 3 h. After the reaction was completed, the resulting mixture was concentrated under vacuum. The residue was purified by Prep-HPLC with the following conditions (Column: YMC-Actus Triart C18 ExRS, 30×150 mm, 5 um; Mobile Phase A: Water (10 mmol/L NH₄HCO₃), Mobile Phase B: ACN; Flow rate: 60 mL/min; Gradient: 34% B to 48% B in 8 min; 254 nm) to afford (1R,5S,6R)—N-[3-(2,6-dimethoxyphenyl)-1H-pyrrolo[2,3-b]pyridin-6-yl]-3-methyl-3-azabicyclo[3.1.0]hexane-6-carboxamide (Compound 177) (15.5 mg, 34%) as a white solid. LCMS (ESI, m/z): [M+H]⁺=393.2. H NMR (300 MHz, DMSO-d₆): δ 11.42 (s, 1H), 10.35 (s, 1H), 7.77 (d, J=8.4 Hz, 1H), 7.52-7.50 (m, 1H), 7.29-7.25 (m, 2H), 6.75 (d, J=8.4 Hz, 2H), 3.69 (s, 6H), 3.00-2.98 (m, 2H), 2.36-2.31 (m, 3H), 2.25 (s, 3H), 1.85 (s, 2H).

### Example S171: Synthesis of (1S,2S)—N-[3-(4-cyclopropoxy-2-methoxypyridin-3-yl)-1H-pyrrolo[2,3-b]pyridin-6-yl]-2-fluorocyclopropane-1-carboxamide (Compound 178)

### Step 1: Synthesis of 3-(6-chloro-1-{[2-(trimethylsilyl)ethoxy]methyl}pyrrolo[2,3-b]pyridin-3-yl)-4-cyclopropoxy-2-methoxypyridine

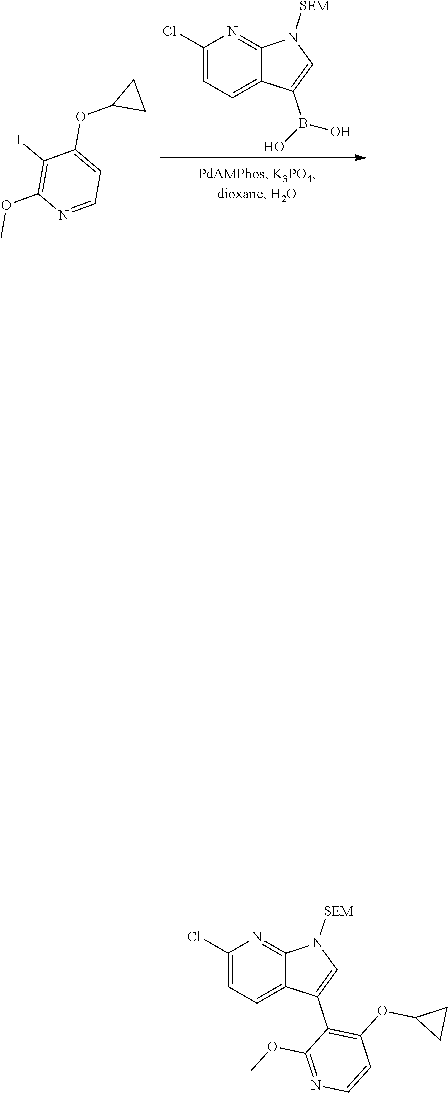 [SMILES: COc1nccc(OC2CC2)c1I ; C[Si](C)(C)CCOCn1cc(B(O)O)c2ccc(Cl)nc21 ; COc1nccc(OC2CC2)c1-c1cn(COCC[Si](C)(C)C)c2nc(Cl)ccc12]

To a solution of 4-cyclopropoxy-3-iodo-2-methoxypyridine (268.0 mg, 0.92 mmol) in 1,4-dioxane/H₂O (8.0 mL/1.6 mL) was added 6-chloro-1-{[2-(trimethylsilyl)ethoxy]methyl}pyrrolo[2,3-b]pyridin-3-ylboronic acid (300.8 mg, 0.92 mmol), K₃PO₄ (390.9 mg, 1.84 mmol) and PdAMPhos (130.4 mg, 0.18 mmol) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 3-(6-chloro-1-{[2-(trimethylsilyl)ethoxy]methyl}pyrrolo[2,3-b]pyridin-3-yl)-4-cyclopropoxy-2-methoxypyridine (253.0 mg, 61%) as a yellow oil. LCMS (ESI, m/z): [M+H]⁺=446.2.

### Step 2: Synthesis of (1S,2S)—N-[3-(4-cyclopropoxy-2-methoxypyridin-3-yl)-1-{[2-(trimethylsilyl)ethoxy]methyl}pyrrolo[2,3-b]pyridin-6-yl]-2-fluorocyclopropane-1-carboxamide

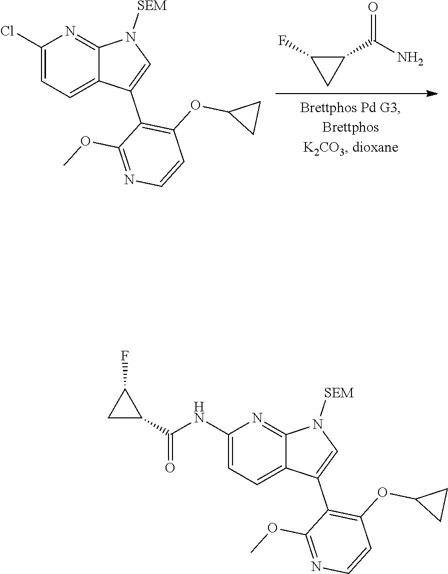 [SMILES: COc1nccc(OC2CC2)c1-c1cn(COCC[Si](C)(C)C)c2nc(Cl)ccc12 ; NC(=O)[C@@H]1C[C@@H]1F ; COc1nccc(OC2CC2)c1-c1cn(COCC[Si](C)(C)C)c2nc(NC(=O)[C@@H]3C[C@@H]3F)ccc12]

To a solution of 3-(6-chloro-1-{[2-(trimethylsilyl)ethoxy]methyl}pyrrolo[2,3-b]pyridin-3-yl)-4-cyclopropoxy-2-methoxypyridine (170.0 mg, 0.38 mmol) in dioxane (6.0 mL) was added (1S,2S)-2-fluorocyclopropane-1-carboxamide (117.9 mg, 1.14 mmol), K₂CO₃ (158.0 mg, 1.14 mmol), Brettphos (40.9 mg, 0.08 mmol) and Brettphos Pd G3 (34.6 mg, 0.04 mmol) at room temperature under N₂. The resulting mixture was stirred at 100° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-[3-(4-cyclopropoxy-2-methoxypyridin-3-yl)-1-{[2-(trimethylsilyl)ethoxy]methyl}pyrrolo[2,3-b]pyridin-6-yl]-2-fluorocyclopropane-1-carboxamide (80.0 mg, 41%) as a yellow oil. LCMS (ESI, m/z): [M+H]⁺=513.2.

### Step 3: Synthesis of (1S,2S)—N-[3-(4-cyclopropoxy-2-methoxypyridin-3-yl)-1H-pyrrolo[2,3-b]pyridin-6-yl]-2-fluorocyclopropane-1-carboxamide (Compound 178)

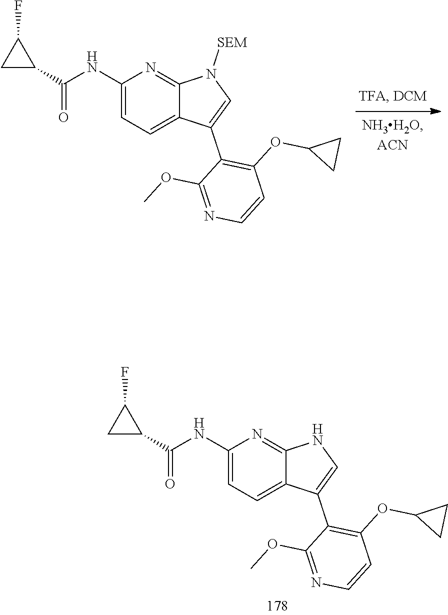 [SMILES: COc1nccc(OC2CC2)c1-c1cn(COCC[Si](C)(C)C)c2nc(NC(=O)[C@@H]3C[C@@H]3F)ccc12 ; COc1nccc(OC2CC2)c1-c1c[nH]c2nc(NC(=O)[C@@H]3C[C@@H]3F)ccc12]

To a solution of (1S,2S)—N-[3-(4-cyclopropoxy-2-methoxypyridin-3-yl)-1-{[2-(trimethylsilyl)ethoxy]methyl}pyrrolo[2,3-b]pyridin-6-yl]-2-fluorocyclopropane-1-carboxamide (80.0 mg, 0.16 mmol) in DCM (3.0 mL) was added TFA (3.0 mL) at room temperature. The resulting mixture was stirred at room temperature for 4 h. The resulting mixture was concentrated under reduced pressure. To the above residue was added NH₃·H₂O (3.0 mL) and ACN (3.0 mL) at room temperature. The resulting mixture was stirred at room temperature for additional 4 h. After the reaction was completed, the mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (4/1, v/v) and then purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 10% B to 40% B in 8 min, 254 nm) to afford (1S,2S)—N-[3-(4-cyclopropoxy-2-methoxypyridin-3-yl)-1H-pyrrolo[2,3-b]pyridin-6-yl]-2-fluorocyclopropane-1-carboxamide (Compound 178) (10.3 mg, 16%) as a white solid. LCMS (ESI, m/z): [M+H]⁺=383.1. ¹H NMR (400 MHz, DMSO-d₆): δ 11.55 (s, 1H), 10.61 (s, 1H), 8.08 (d, J=6.0 Hz, 1H), 7.84 (d, J=8.4 Hz, 1H), 7.58 (d, J=8.4 Hz, 1H), 7.36 (d, J=2.4 Hz, 1H), 7.14 (d, J=5.6 Hz, 1H), 5.02-4.83 (m, 1H), 3.95-3.92 (m, 1H), 3.82 (s, 3H), 2.28-2.19 (m, 1H), 1.72-1.55 (m, 1H), 1.16-1.13 (m, 1H), 0.80-0.76 (m, 2H), 0.66-0.61 (m, 2H).

### Example S172: Synthesis of 3-[3-(dimethylamino)propyl]-1-(3-{5-methoxy-[1,3]thiazolo[4,5-b]pyridin-6-yl}-1H-pyrrolo[2,3-b]pyridin-6-yl)urea (Compound 179)

### Step 1: Synthesis of 3-[3-(dimethylamino)propyl]-1-(3-{5-methoxy-[1,3]thiazolo[4,5-b]pyridin-6-yl}-1-{[2-(trimethylsilyl)ethoxy]methyl}pyrrolo[2,3-b]pyridin-6-yl)urea

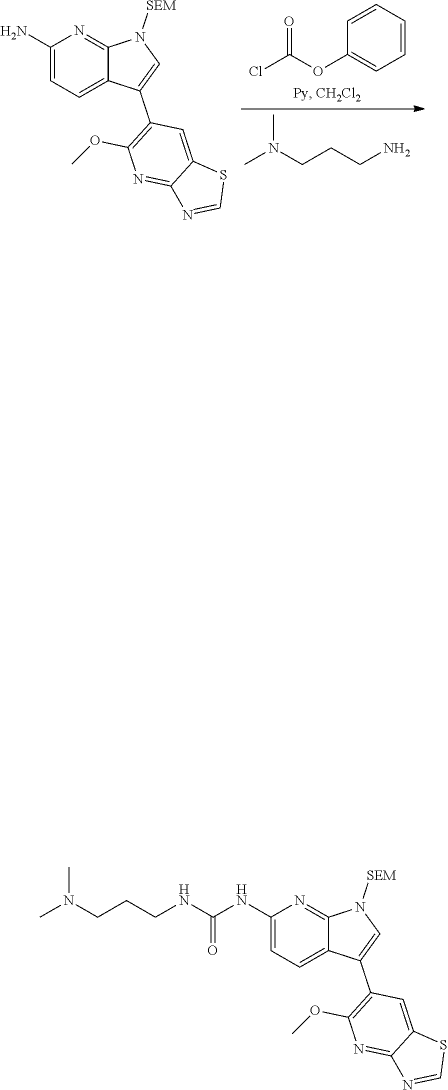

To a solution of 3-{5-methoxy-[1,3]thiazolo[4,5-b]pyridin-6-yl}-1-{[2-(trimethylsilyl)ethoxy]methyl}pyrrolo[2,3-b]pyridin-6-amine (170.0 mg, 0.40 mmol) in CH₂Cl₂ (5.0 mL) was added pyridine (125.8 mg, 1.59 mmol) and phenyl chloroformate (74.7 mg, 0.48 mmol) at 0° C. The mixture was stirred at room temperature for 1 h. The mixture was concentrated under vacuum. To the above residue was added pyridine (3.0 mL) and N₁,N₁-dimethylpropane-1,3-diamine (121.9 mg, 1.19 mmol) at room temperature. The resulting mixture was stirred at 60° C. for 3 h. After the reaction was completed, the resulting mixture was concentrated under vacuum. The residue was purified by flash column chromatography with CH₂Cl₂/MeOH (85/15, v/v) to afford 3-[3-(dimethylamino)propyl]-1-(3-{5-methoxy-[1,3]thiazolo[4,5-b]pyridin-6-yl}-1-{[2-(trimethylsilyl)ethoxy]methyl}pyrrolo[2,3-b]pyridin-6-yl)urea (100.0 mg, 45%) as a yellow solid. LCMS (ESI, m/z): [M+H]⁺=556.2.

### Step 2: Synthesis of 3-[3-(dimethylamino)propyl]-1-(3-{5-methoxy-[1,3]thiazolo[4,5-b]pyridin-6-yl}-1H-pyrrolo[2,3-b]pyridin-6-yl)urea (Compound 179)

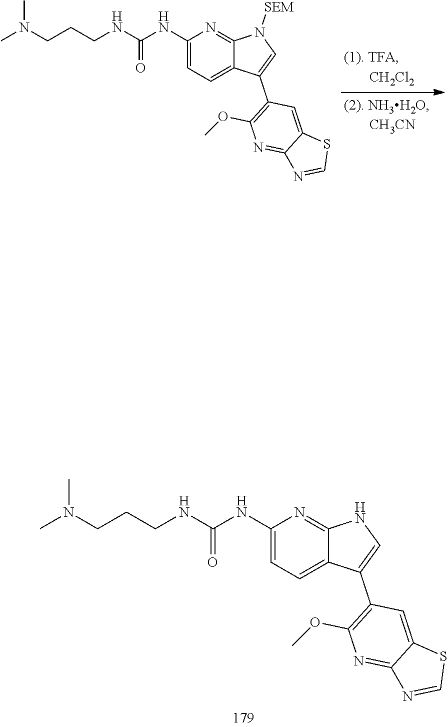 [SMILES: COc1nc2ncsc2cc1-c1cn(COCC[Si](C)(C)C)c2nc(NC(=O)NCCCN(C)C)ccc12 ; COc1nc2ncsc2cc1-c1c[nH]c2nc(NC(=O)NCCCN(C)C)ccc12]

To a solution of 3-[3-(dimethylamino)propyl]-1-(3-{5-methoxy-[1,3]thiazolo[4,5-b]pyridin-6-yl}-1-{[2-(trimethylsilyl)ethoxy]methyl}pyrrolo[2,3-b]pyridin-6-yl)urea (80.0 mg, 0.14 mmol) in CH₂Cl₂ (2.0 mL) was added TFA (2.0 mL) at room temperature. The mixture was stirred at room temperature for 2 h. The mixture was concentrated under vacuum. To the above residue were added ACN (2.0 mL) and NH₃·H₂O (2.0 mL) at room temperature. The resulting mixture was stirred at room temperature for another 2 h. After the reaction was completed, the resulting mixture was concentrated under vacuum. The residue was purified by Prep-HPLC with the following conditions (Column: YMC-Actus Triart C18 ExRS, 30×250 mm, 5 um; Mobile Phase A: Water (10 mmol/L NH₄HCO₃), Mobile Phase B: ACN; Flow rate: 60 mL/min; Gradient: 20% B to 30% B in 11 min; 254 nm) to afford 3-[3-(dimethylamino)propyl]-1-(3-{5-methoxy-[1,3]thiazolo[4,5-b]pyridin-6-yl}-1H-pyrrolo[2,3-b]pyridin-6-yl)urea (Compound 179) (19.8 mg, 32%) as a white solid. LCMS (ESI, m/z): [M+H]⁺=426.3. ¹H NMR (400 MHz, DMSO-d₆): δ 11.71 (s, 1H), 9.51 (s, 1H), 9.19 (s, 1H), 8.78 (s, 1H), 8.45 (s, 1H), 8.16 (d, J=8.4 Hz, 1H), 7.73 (d, J=2.4 Hz, 1H), 7.12 (d, J=8.8 Hz, 1H), 4.06 (s, 3H), 3.27-3.22 (m, 2H), 2.34-2.27 (m, 2H), 2.14 (s, 6H), 1.70-1.64 (m, 2H).

### Example S173: Synthesis of (1S,2S)—N-(3-(2,6-dimethoxyphenyl)-1H-pyrrolo[2,3-b]pyridin-6-yl)-2-fluoro-2-(hydroxymethyl)cyclopropanecarboxamide and (1R,2R)—N-(3-(2,6-dimethoxyphenyl)-1H-pyrrolo[2,3-b]pyridin-6-yl)-2-fluoro-2-(hydroxymethyl)cyclopropanecarboxamide (Compound 180 and Compound 181)

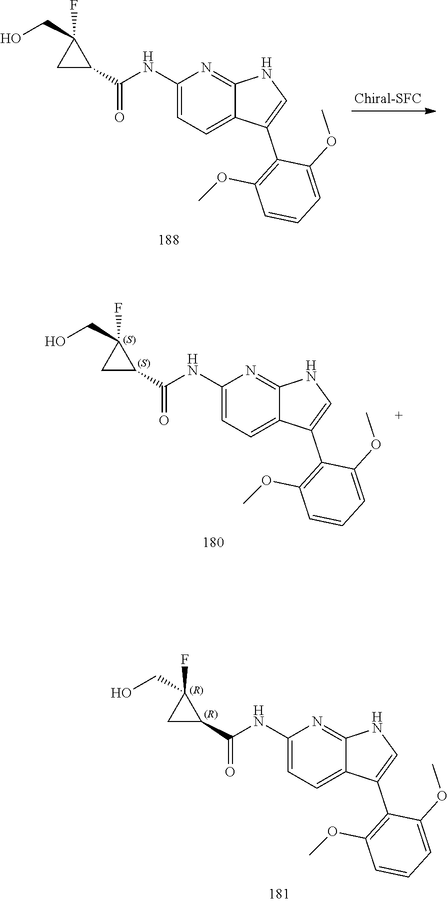

The cis-N-(3-(2,6-dimethoxyphenyl)-1H-pyrrolo[2,3-b]pyridin-6-yl)-2-fluoro-2-(hydroxymethyl)cyclopropanecarboxamide (94.0 mg, 0.24 mmol) was separated by CHIRAL-HPLC with the following conditions (Column: CHIRAL ART Amylose-SA, 2×25 cm, 5 m; Mobile Phase A: Hex (0.5% 2M NH₃-MeOH)—HPLC, Mobile Phase B: EtOH—HPLC; Flow rate: 20 mL/min; Gradient: 50% B to 50% B in 17 min; Wave Length: 220/254 nm) to afford N-(3-(2,6-dimethoxyphenyl)-1H-pyrrolo[2,3-b]pyridin-6-yl)-2-fluoro-2-(hydroxymethyl)cyclopropanecarboxamide Enantiomer 1 (18.2 mg, 19%) as a white solid and N-(3-(2,6-dimethoxyphenyl)-1H-pyrrolo[2,3-b]pyridin-6-yl)-2-fluoro-2-(hydroxymethyl)cyclopropanecarboxamide Enantiomer 2 (22.6 mg, 24%) as a white solid. The absolute stereochemistry of Enantiomers 1 and 2 was not assigned. The two enantiomeric structures that could be obtained from chiral separation of the enantiomeric mixture as described above are shown as Compounds 180 and 181 in Table 1.

N-(3-(2,6-dimethoxyphenyl)-1H-pyrrolo[2,3-b]pyridin-6-yl)-2-fluoro-2-(hydroxymethyl)cyclopropanecarboxamide Enantiomer 1: Retention Time 1=7.02 min; LCMS (ESI, m/z): [M+H]⁺=386.1. ¹H NMR (400 MHz, DMSO-d₆): δ 11.43 (d, J=1.6 Hz, 1H), 10.43 (s, 1H), 7.82 (d, J=8.4 Hz, 1H), 7.55 (d, J=8.8 Hz, 1H), 7.30-7.26 (m, 2H), 6.75 (d, J=8.4 Hz, 2H), 5.15-5.12 (m, 1H), 3.72-3.66 (m, 8H), 2.33-2.29 (m, 1H), 1.76-1.68 (m, 1H), 1.25-1.22 (m, 1H).

N-(3-(2,6-dimethoxyphenyl)-1H-pyrrolo[2,3-b]pyridin-6-yl)-2-fluoro-2-(hydroxymethyl)cyclopropanecarboxamide Enantiomer 2: Retention Time 2=14.64 min; LCMS (ESI, m/z): [M+H]⁺=386.3. H NMR (400 MHz, DMSO-d₆): δ 11.41 (s, 1H), 10.39 (s, 1H), 7.81 (d, J=8.0 Hz, 1H), 7.55 (d, J=8.4 Hz, 1H), 7.30-7.26 (m, 2H), 6.75 (d, J=8.0 Hz, 2H), 5.13-5.10 (m, 1H), 3.73-3.66 (m, 8H), 2.33-2.29 (m, 1H), 1.77-1.69 (m, 1H), 1.29-1.18 (m, 1H).

### Example S174: Synthesis of (1S,2S)-2-fluoro-N-[3-(2-methoxy-6-methylphenyl)-1H-pyrazolo[3,4-b]pyridin-6-yl]cyclopropane-1-carboxamide (Compound 182)

### Step 1: Synthesis of 6-chloro-3-(2-methoxy-6-methylphenyl)-1-{[2-(trimethylsilyl)ethoxy]methyl}pyrazolo[3,4-b]pyridine

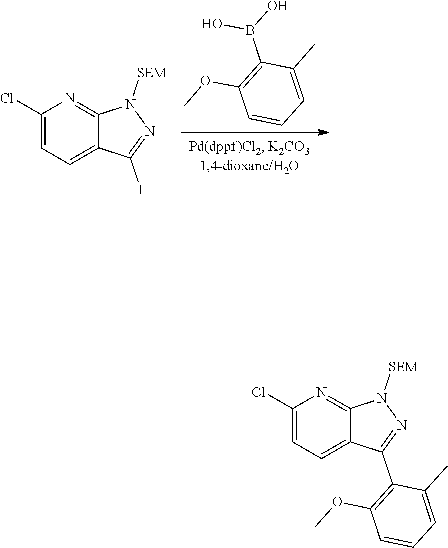

To a solution of 6-chloro-3-iodo-1-{[2-(trimethylsilyl)ethoxy]methyl}pyrazolo[3,4-b]pyridine (400.0 mg, 0.98 mmol) in dioxane/H₂O (10.0 mL/2.0 mL) was added 2-methoxy-6-methylphenylboronic acid (162.0 mg, 0.97 mmol), K₂CO₃ (404.8 mg, 2.93 mmol) and Pd(dppf)Cl₂ (71.4 mg, 0.10 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2-methoxy-6-methylphenyl)-1-{[2-(trimethylsilyl)ethoxy]methyl}pyrazolo[3,4-b]pyridine (120.0 mg, 30%) as a colorless oil. LCMS (ESI, m/z): [M+H]⁺=404.1.

### Step 2: Synthesis of (1S,2S)-2-fluoro-N-[3-(2-methoxy-6-methylphenyl)-1-{[2-(trimethylsilyl)ethoxy]methyl}pyrazolo[3,4-b]pyridin-6-yl]cyclopropane-1-carboxamide

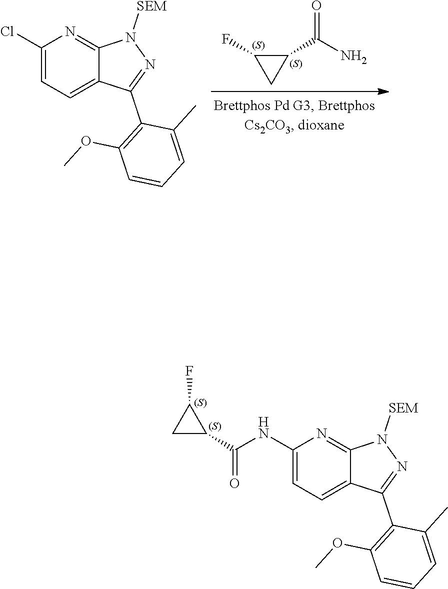

To a solution of 6-chloro-3-(2-methoxy-6-methylphenyl)-1-{[2-(trimethylsilyl)ethoxy]methyl}pyrazolo[3,4-b]pyridine (210.0 mg, 0.52 mmol) in 1,4-dioxane (16.0 mL) was added (1S,2S)-2-fluorocyclopropane-1-carboxamide (267.9 mg, 2.60 mmol), BrettPhos (55.8 mg, 0.10 mmol), Cs₂CO₃ (508.1 mg, 1.56 mmol) and BrettPhos Pd G3 (47.1 mg, 0.05 mmol) at room temperature under N₂. The resulting mixture was stirred at 100° C. for 4 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)-2-fluoro-N-[3-(2-methoxy-6-methylphenyl)-1-{[2-(trimethylsilyl)ethoxy]methyl}pyrazolo[3,4-b]pyridin-6-yl]cyclopropane-1-carboxamide (120.0 mg, 49%) as a yellow oil. LCMS (ESI, m/z): [M+H]⁺=471.2.

### Step 3: Synthesis of (1S,2S)-2-fluoro-N-[3-(2-methoxy-6-methylphenyl)-1H-pyrazolo[3,4-b]pyridin-6-yl]cyclopropane-1-carboxamide (Compound 182)

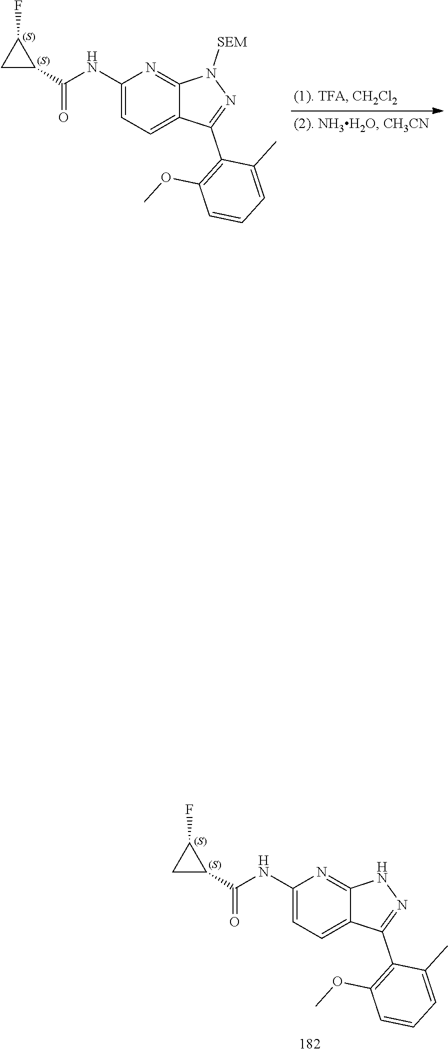

To a solution of (1S,2S)-2-fluoro-N-[3-(2-methoxy-6-methylphenyl)-1-{[2-(trimethylsilyl)ethoxy]methyl}pyrazolo[3,4-b]pyridin-6-yl]cyclopropane-1-carboxamide (120.0 mg 0.12 mmol) in CH₂Cl₂ (10.0 mL) was added TFA (10.0 mL) at room temperature. The resulting mixture was stirred at room temperature for 4 h. The resulting mixture was concentrated under reduced pressure. To the residue in CH₃CN (8.0 mL) was added NH₃·H₂O (8.0 mL) at room temperature. The resulting mixture was stirred at room temperature for 16 h. After the reaction was completed, the resulting mixture was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 33% B to 43% B in 8 min; 254 nm) to afford (1S,2S)-2-fluoro-N-[3-(2-methoxy-6-methylphenyl)-1H-pyrazolo[3,4-b]pyridin-6-yl]cyclopropane-1-carboxamide (Compound 182) (16.9 mg, 14%) as a white solid. LCMS (ESI, m/z): [M+H]⁺=341.2. ¹H NMR (400 MHz, DMSO-d₆): δ 13.39 (s, 1H), 10.96 (s, 1H), 7.93 (d, J=8.8 Hz, 1H), 7.78 (d, J=8.8 Hz, 1H), 7.36-7.32 (m, 1H), 7.00-6.94 (m, 2H), 5.05-4.85 (m, 1H), 3.66 (s, 3H), 2.28-2.25 (m, 1H), 2.12 (s, 3H), 1.70-1.62 (m, 1H), 1.23-1.16 (m, 1H).

### Example S175: Synthesis of trans-N-(3-(2,6-dimethoxyphenyl)-1H-pyrrolo[2,3-b]pyridin-6-yl)-2-fluoro-2-(hydroxymethyl)cyclopropanecarboxamide (Compound 183)

### Step 1: Synthesis of trans-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide

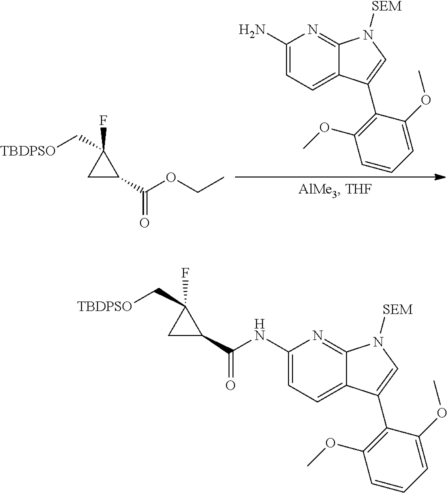

To a solution of 3-(2,6-dimethoxyphenyl)-1-((2-(trimethylsilyl)ethoxy)methyl)-1H-pyrrolo[2,3-b]pyridin-6-amine (300.0 mg, 0.75 mmol) in THE (10.0 mL) was added trans-ethyl 2-((tert-butyldiphenylsilyloxy)methyl)-2-fluorocyclopropanecarboxylate (902 mg, 2.25 mmol) and AlMe₃ (1.9 mL, 2 mol/L) at 0° C. under N₂. The resulting mixture was stirred at 80° C. for 5 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (30/70, v/v) to afford trans-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide (200.0 mg, 35%) as a yellow solid. LCMS (ESI, m/z): [M+H]⁺=754.3.

### Step 2: Synthesis of trans-N-(3-(2,6-dimethoxyphenyl)-1H-pyrrolo[2,3-b]pyridin-6-yl)-2-fluoro-2-(hydroxymethyl)cyclopropanecarboxamide (Compound 183)

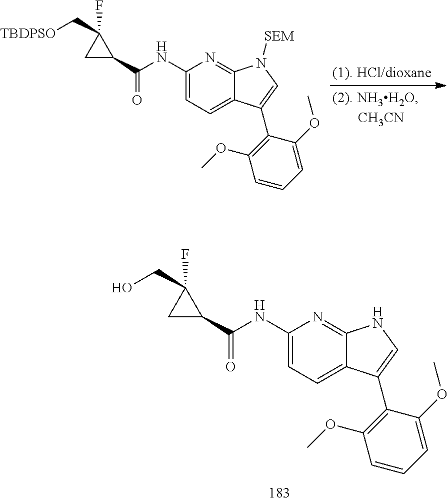

The solution of trans-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide (150.0 mg, 0.20 mmol) in HCl/dioxane (5.0 mL, 4 mol/L) was stirred at room temperature for 16 h. The resulting mixture was concentrated under reduced pressure. To the residue in ACN (5.0 mL) was added NH₃·H₂O (5.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with petroleum MeOH/H₂O (70/30, v/v) to afford trans-N-(3-(2,6-dimethoxyphenyl)-1H-pyrrolo[2,3-b]pyridin-6-yl)-2-fluoro-2-(hydroxymethyl)cyclopropanecarboxamide (Compound 183) (1.2 mg, 2%) as a white solid. LCMS (ESI, m/z): [M+H]⁺=386.3. ¹H NMR (400 MHz, DMSO-d₆): δ 11.47 (s, 1H), 10.64 (s, 1H), 7.80 (d, J=8.4 Hz, 1H), 7.55 (d, J=8.8 Hz, 1H), 7.30-7.26 (m, 2H), 6.75 (d, J=8.4 Hz, 2H), 5.06-5.03 (m, 1H), 3.98-3.91 (m, 1H), 3.86-3.74 (m, 1H) 3.69 (s, 6H) 2.69-2.62 (m, 1H), 1.53-1.47 (m, 1H), 1.35-1.30 (m, 1H).

### Example S176: Synthesis of 1-[2-(4-cyano-1-ethylpiperidin-4-yl)ethyl]-3-[3-(2-methoxyphenyl)-1H-pyrrolo[2,3-b]pyridin-6-yl]urea (Compound 184)

### Step 1: Synthesis of 1-ethylpiperidine-4-carbonitrile

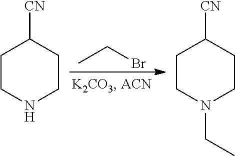

To a mixture of piperidine-4-carbonitrile (1.0 g, 9.07 mmol) and K₂CO₃ (3.7 g, 27.20 mmol) in MeCN (10.0 mL) was added bromoethane (1.0 g, 9.08 mmol) at room temperature. The resulting mixture was stirred at 60° C. for 16 h. After the reaction was completed, the resulting mixture was concentrated under vacuum. The residue was purified by flash column chromatography with petroleum ether/ethyl acetate (5/1, v/v) to afford 1-ethylpiperidine-4-carbonitrile (750.0 mg, 59%) as a yellow oil. LCMS (ESI, m/z): [M+H]⁺=139.1

### Step 2: Synthesis of 4-[2-[(tert-butyldimethylsilyl)oxy]ethyl]-1-ethylpiperidine-4-carbonitrile

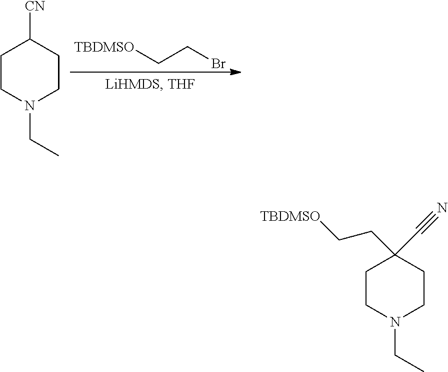

To a solution of 1-ethylpiperidine-4-carbonitrile (2.2 g, 15.91 mmol) in THE (20.0 mL) was added LiHMDS (31.0 mL, 1 mol/L) at −10° C. under N₂. The resulting mixture was stirred at −10° C. for 1 h under N₂. Then 2-bromoethoxy(tert-butyl)dimethylsilane (7.6 g, 31.85 mmol) was added dropwise to the mixture at −10° C. The resulting mixture was stirred at room temperature for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 4-[2-[(tert-butyldimethylsilyl)oxy]ethyl]-1-ethylpiperidine-4-carbonitrile (3.0 g, 63%) as a yellow oil. LCMS (ESI, m/z): [M+H]⁺=297.2

### Step 3: Synthesis of 1-ethyl-4-(2-hydroxyethyl)piperidine-4-carbonitrile

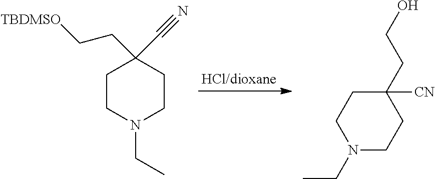 [SMILES: CCN1CCC(C#N)(CCO[Si](C)(C)C(C)(C)C)CC1 ; CCN1CCC(C#N)(CCO)CC1]

The solution of 4-[2-[(tert-butyldimethylsilyl)oxy]ethyl]-1-ethylpiperidine-4-carbonitrile (3.0 g, 10.11 mmol) in HCl/1,4-dioxane (20.0 mL, 4 mol/L) was stirred at room temperature for 2 h. After the reaction was completed, the reaction mixture was evaporated in vacuo. The PH of the residue was adjusted to 8 with NaHCO₃ (aq) and then extracted with ethyl acetate. The aqueous layer was concentrated under reduced pressure to afford 1-ethyl-4-(2-hydroxyethyl)piperidine-4-carbonitrile (2.3 g, crude) as yellow solid. LCMS (ESI, m/z): [M+H]⁺=183.1

### Step 4: Synthesis of 4-(2-azidoethyl)-1-ethylpiperidine-4-carbonitrile

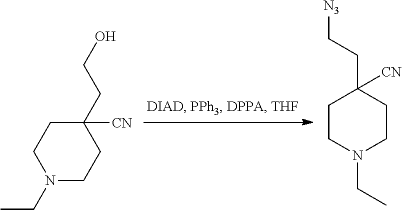

To a mixture of 1-ethyl-4-(2-hydroxyethyl)piperidine-4-carbonitrile (300.0 mg, 1.64 mmol) and PPh₃ (863.4 mg, 3.29 mmol) in THE (5.0 mL) was added DIAD (665.6 mg, 3.29 mmol) at 0° C. under N₂. The resulting mixture was stirred at 0° C. for 15 min under N₂. Then DPPA (905.9 mg, 3.29 mmol) was added to the mixture at 0° C. The resulting mixture was stirred at room temperature for 16 h. After the reaction was completed, the resulting mixture was concentrated under reduced pressure. The residue was purified by flash column chromatography with CH₂Cl₂/MeOH (10/1, v/v) to afford 4-(2-azidoethyl)-1-ethylpiperidine-4-carbonitrile (300.0 mg, 87%) as a colorless oil. LCMS (ESI, m/z): [M+H]⁺=208.1

### Step 5: Synthesis of 4-(2-aminoethyl)-1-ethylpiperidine-4-carbonitrile

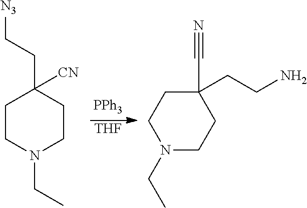

To a solution of 4-(2-azidoethyl)-1-ethylpiperidine-4-carbonitrile (1.4 g, 6.75 mmol) in THE (20.0 mL) was added triphenylphosphine (2.6 g, 10.13 mmol) at room temperature. The resulting mixture was stirred at 70° C. for 2 h. After the reaction was completed, the resulting mixture was diluted with H₂O and extracted with ethyl acetate. The aqueous layer was concentrated under reduced pressure to afford 4-(2-aminoethyl)-1-ethylpiperidine-4-carbonitrile (1.1 g, crude) as a yellow oil. LCMS (ESI, m/z): [M+H]⁺=182.2

### Step 6: Synthesis of 1-[2-(4-cyano-1-ethylpiperidin-4-yl)ethyl]-3-[3-(2-methoxyphenyl)-1-[[2-(trimethylsilyl)ethoxy]methyl]pyrrolo[2,3-b]pyridin-6-yl]urea

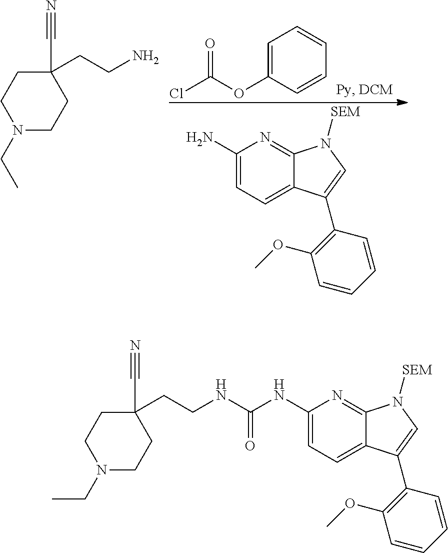

To a mixture of 3-(2-methoxyphenyl)-1-[[2-(trimethylsilyl)ethoxy]methyl]pyrrolo[2,3-b]pyridin-6-amine (1.0 g, 2.75 mmol) and pyridine (872.6 mg, 11.03 mmol) in DCM (10.0 mL) was added phenyl chloroformate (518.2 mg, 3.31 mmol) at 0° C. The resulting mixture was stirred for at room temperature 2 h. The resulting mixture was concentrated under reduced pressure. To the above mixture was added pyridine (10.0 mL) and 4-(2-aminoethyl)-1-ethylpiperidine-4-carbonitrile (500.0 mg, 2.75 mmol) at room temperature. The resulting mixture was stirred at 70° C. for 16 h. After the reaction was completed, the resulting mixture was concentrated under reduced pressure. The residue was purified by flash column chromatography with CH₂Cl₂/MeOH (8/1, v/v) to afford 1-[2-(4-cyano-1-ethylpiperidin-4-yl)ethyl]-3-[3-(2-methoxyphenyl)-1-[[2-(trimethylsilyl)ethoxy]methyl]pyrrolo[2,3-b]pyridin-6-yl]urea (450.0 mg, 28%) as a yellow solid. LCMS (ESI, m/z): [M+H]⁺=577.3

### Step 7: Synthesis of 1-[2-(4-cyano-1-ethylpiperidin-4-yl)ethyl]-3-[3-(2-methoxyphenyl)-1H-pyrrolo[2,3-b]pyridin-6-yl]urea (Compound 184)

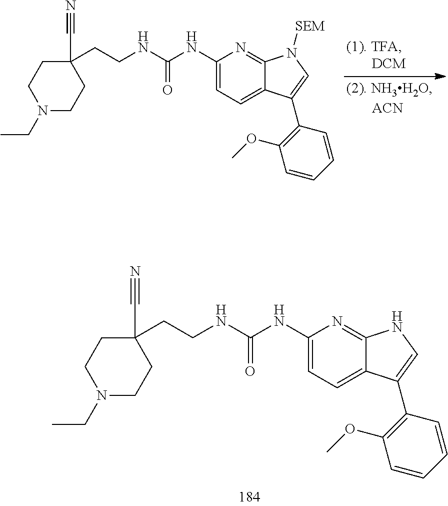 [SMILES: CCN1CCC(C#N)(CCNC(=O)Nc2ccc3c(-c4ccccc4OC)cn(COCC[Si](C)(C)C)c3n2)CC1 ; CCN1CCC(C#N)(CCNC(=O)Nc2ccc3c(-c4ccccc4OC)c[nH]c3n2)CC1]

To a solution of 1-[2-(4-cyano-1-methylpiperidin-4-yl)ethyl]-3-[3-(2-methoxyphenyl)-1-[[2-(trimethylsilyl)ethoxy]methyl]pyrrolo[2,3-b]pyridin-6-yl]urea (450.0 mg, 0.80 mmol) in DCM (5.0 mL) was added TFA (2.0 mL) at room temperature. The resulting mixture was concentrated under reduced pressure. To the above mixture was added NH₃·H₂O (2.0 mL) and ACN (2.0 mL) at room temperature. The resulting mixture was stirred at room temperature for another 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 11% B to 20% B in 8 min, 254/220 nm) to afford 1-[2-(4-cyano-1-ethylpiperidin-4-yl)ethyl]-3-[3-(2-methoxyphenyl)-1H-pyrrolo[2,3-b]pyridin-6-yl]urea (Compound 184) (77.3 mg, 22%) as a yellow solid. LCMS (ESI, m/z): [M+H]⁺=447.2. ¹H NMR (300 MHz, DMSO-d₆): δ 11.41 (s, 1H), 9.42 (s, 1H), 7.99-7.89 (m, 2H), 7.56-7.52 (m, 2H), 7.30-7.24 (m, 1H), 7.11 (d, J=7.5 Hz, 1H), 7.05-7.00 (m, 1H), 3.82 (s, 3H), 3.62-3.48 (m, 2H), 3.34-2.73 (m, 3H), 2.08-1.76 (m, 6H), 1.29-1.19 (m, 3H).

### Example S177. Synthesis of (1R,2S)—N-[3-(2-methoxyphenyl)-1H-pyrrolo[2,3-b]pyridin-6-yl]-2-[(4-methylpiperazin-1-yl)methyl]cyclopropane-1-carboxamide and (1S,2R)—N-[3-(2-methoxyphenyl)-1H-pyrrolo[2,3-b]pyridin-6-yl]-2-[(4-methylpiperazin-1-yl)methyl]cyclopropane-1-carboxamide (Compound 185 and Compound 186)

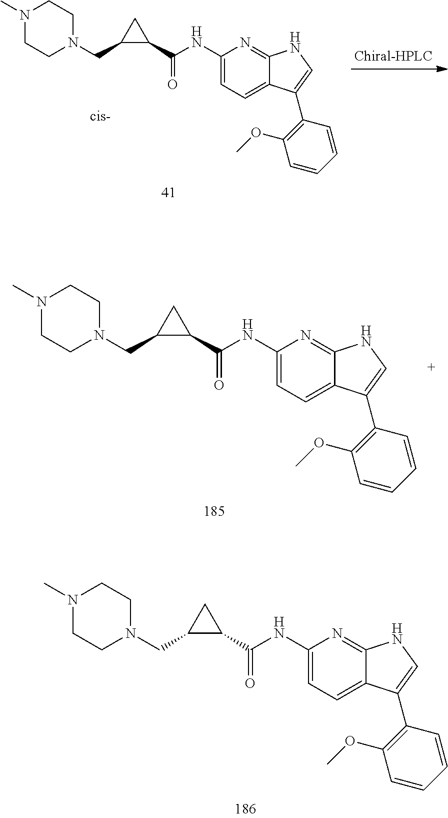 [SMILES: COc1ccccc1-c1c[nH]c2nc(NC(=O)[C@@H]3C[C@@H]3CN3CCN(C)CC3)ccc12 ; COc1ccccc1-c1c[nH]c2nc(NC(=O)[C@@H]3C[C@@H]3CN3CCN(C)CC3)ccc12 ; COc1ccccc1-c1c[nH]c2nc(NC(=O)[C@H]3C[C@H]3CN3CCN(C)CC3)ccc12]

The cis-N-[3-(2-methoxyphenyl)-1H-pyrrolo[2,3-b]pyridin-6-yl]-2-[(4-methylpiperazin-1-yl)methyl]cyclopropane-1-carboxamide (Compound 41) (33.0 mg, 0.08 mmol) was separated by CHIRAL-HPLC with the following conditions (Column: CHIRALPAK IH, 2×25 cm, 5 um; Mobile Phase A: MTBE (0.5% 2M NH₃-MeOH)—HPLC, Mobile Phase B: EtOH—HPLC; Flow rate: 20 mL/min; Gradient: 10% B to 10% B in 22 min; Wave Length: 254/220 nm) to afford N-[3-(2-methoxyphenyl)-1H-pyrrolo[2,3-b]pyridin-6-yl]-2-[(4-methylpiperazin-1-yl)methyl]cyclopropane-1-carboxamide Enantiomer 1 (10.4 mg, 31%) as a white solid and N-[3-(2-methoxyphenyl)-1H-pyrrolo[2,3-b]pyridin-6-yl]-2-[(4-methylpiperazin-1-yl)methyl]cyclopropane-1-carboxamide Enantiomer 2 (10.0 mg, 30%) as a white solid. The absolute stereochemistry of Enantiomers 1 and 2 was not assigned. The two enantiomeric structures that could be obtained from chiral separation of the enantiomeric mixture as described above are shown as Compounds 185 and 186 in Table 1.

N-[3-(2-methoxyphenyl)-1H-pyrrolo[2,3-b]pyridin-6-yl]-2-[(4-methylpiperazin-1-yl)methyl]cyclopropane-1-carboxamide Enantiomer 1: Retention Time 1: 7.772 min; LCMS (ESI, m/z): [M+H]⁺=420.3. ¹H NMR (300 MHz, DMSO-d₆): δ 11.53 (d, J=2.1 Hz, 1H), 10.51 (s, 1H), 7.97 (d, J=8.7 Hz, 1H), 7.88 (d, J=8.7 Hz, 1H), 7.56-7.52 (m, 2H), 7.30-7.25 (m, 1H), 7.11 (d, J=7.5 Hz, 1H), 7.05-7.00 (m, 1H), 3.82 (s, 3H), 2.60-2.52 (m, 2H), 2.51-2.46 (m, 1H), 2.44-2.32 (m, 3H), 2.31-2.17 (m, 4H), 2.16-2.10 (m, 1H), 2.08 (s, 3H), 1.35-1.30 (m, 1H), 1.03-0.99 (m, 1H), 0.91-0.82 (m, 1H).

N-[3-(2-methoxyphenyl)-1H-pyrrolo[2,3-b]pyridin-6-yl]-2-[(4-methylpiperazin-1-yl)methyl]cyclopropane-1-carboxamide Enantiomer 2: Retention Time 2:17.229 min; LCMS (ESI, m/z): [M+H]⁺=420.3. ¹H NMR (300 MHz, DMSO-d₆): δ 11.53 (d, J=1.8 Hz, 1H), 10.51 (s, 1H), 7.97 (d, J=8.7 Hz, 1H), 7.88 (d, J=8.7 Hz, 1H), 7.56-7.52 (m, 2H), 7.30-7.25 (m, 1H), 7.11 (d, J=7.5 Hz, 1H), 7.05-7.00 (m, 1H), 3.82 (s, 3H), 2.60-2.52 (m, 2H), 2.51-2.46 (m, 1H), 2.44-2.32 (m, 3H), 2.31-2.17 (m, 4H), 2.16-2.10 (m, 1H), 2.08 (s, 3H), 1.38-1.31 (m, 1H), 1.03-0.99 (m, 1H), 0.91-0.82 (m, 1H).

### Example S178: Synthesis of 1-[3-(2,6-dimethoxyphenyl)-1H-pyrrolo[2,3-b]pyridin-6-yl]-3-[(3-fluoro-1-methylazetidin-3-yl)methyl]urea (Compound 187)

### Step 1: Synthesis of tert-butyl 3-[({[3-(2,6-dimethoxyphenyl)-1-{[2-(trimethylsilyl)ethoxy]methyl}pyrrolo[2,3-b]pyridin-6-yl]carbamoyl}amino)methyl]-3-fluoroazetidine-1-carboxylate

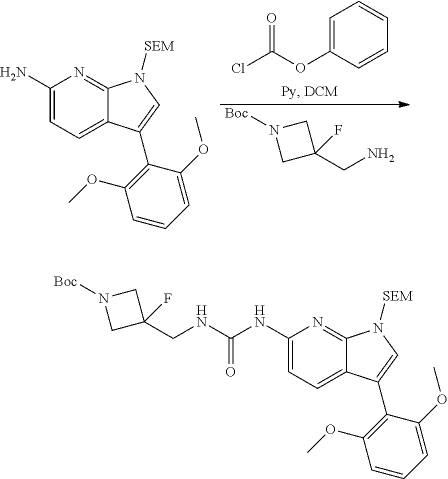

To a solution of 3-(2,6-dimethoxyphenyl)-1-{[2-(trimethylsilyl)ethoxy]methyl}pyrrolo[2,3-b]pyridin-6-amine (450.0 mg, 1.13 mmol) in DCM (10.0 mL) was added pyridine (356.3 mg, 4.50 mmol) and phenyl chloroformate (211.6 mg, 1.35 mmol) at room temperature. The resulting mixture was stirred at room temperature for 16 h. The resulting mixture was concentrated under reduced pressure. To the residue in pyridine (10 mL) was added tert-butyl 3-(aminomethyl)-3-fluoroazetidine-1-carboxylate (690.1 mg, 3.38 mmol) at room temperature. The resulting mixture was stirred at 60° C. for 4 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford tert-butyl 3-[({[3-(2,6-dimethoxyphenyl)-1-{[2-(trimethylsilyl)ethoxy]methyl}pyrrolo[2,3-b]pyridin-6-yl]carbamoyl}amino)methyl]-3-fluoroazetidine-1-carboxylate (600.0 mg, 85%) as a yellow oil. LCMS (ESI, m/z): [M+H]⁺=630.3.

### Step 2: Synthesis of 1-[3-(2,6-dimethoxyphenyl)-1-{[2-(trimethylsilyl)ethoxy]methyl}pyrrolo[2,3-b]pyridin-6-yl]-3-[(3-fluoroazetidin-3-yl)methyl]urea

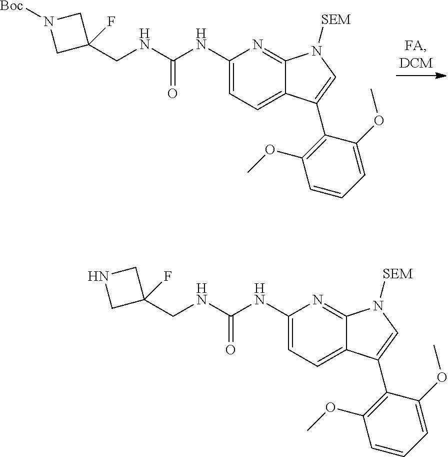 [SMILES: COc1cccc(OC)c1-c1cn(COCC[Si](C)(C)C)c2nc(NC(=O)NCC3(F)CN(C(=O)OC(C)(C)C)C3)ccc12 ; COc1cccc(OC)c1-c1cn(COCC[Si](C)(C)C)c2nc(NC(=O)NCC3(F)CNC3)ccc12]

To a solution of tert-butyl 3-[({[3-(2,6-dimethoxyphenyl)-1-{[2-(trimethylsilyl)ethoxy]methyl}pyrrolo[2,3-b]pyridin-6-yl]carbamoyl}amino)methyl]-3-fluoroazetidine-1-carboxylate (580.0 mL, 0.92 mmol) in DCM (5.0 mL) was added HCOOH (10.0 mL) at room temperature. The resulting mixture was stirred at room temperature for 16 h. After the reaction was completed, the pH value of the mixture was adjusted to 8 with NaHCO₃ (aq). The mixture was extracted with EtOAc. The combined organic layer was washed with brine, dried over anhydrous sodium sulfate and filtered. The filtrate was concentrated under reduced pressure to afford 1-[3-(2,6-dimethoxyphenyl)-1-{[2-(trimethylsilyl)ethoxy]methyl}pyrrolo[2,3-b]pyridin-6-yl]-3-[(3-fluoroazetidin-3-yl)methyl]urea (420.0 mg, crude) as a yellow oil. LCMS (ESI, m/z): [M+H]⁺=530.3.

### Step 3: Synthesis of 1-[3-(2,6-dimethoxyphenyl)-1-{[2-(trimethylsilyl)ethoxy]methyl}pyrrolo[2,3-b]pyridin-6-yl]-3-[(3-fluoro-1-methylazetidin-3-yl)methyl]urea

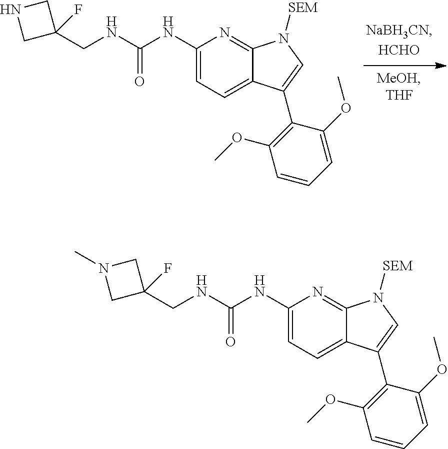 [SMILES: COc1cccc(OC)c1-c1cn(COCC[Si](C)(C)C)c2nc(NC(=O)NCC3(F)CNC3)ccc12 ; COc1cccc(OC)c1-c1cn(COCC[Si](C)(C)C)c2nc(NC(=O)NCC3(F)CN(C)C3)ccc12]

To a solution of 1-[3-(2,6-dimethoxyphenyl)-1-{[2-(trimethylsilyl)ethoxy]methyl}pyrrolo[2,3-b]pyridin-6-yl]-3-[(3-fluoroazetidin-3-yl)methyl]urea (420.0 mg, crude) in THF/CH₃OH (20.0 mL/4.0 mL) was added NaBH₃CN (149.5 mg, 2.38 mmol) and HCHO (238.1 mg, 7.93 mmol) at room temperature. The resulting mixture was stirred at room temperature for 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure to afford 1-[3-(2,6-dimethoxyphenyl)-1-{[2-(trimethylsilyl)ethoxy]methyl}pyrrolo[2,3-b]pyridin-6-yl]-3-[(3-fluoro-1-methylazetidin-3-yl)methyl]urea (400.0 mg, crude) as a white solid. LCMS (ESI, m/z): [M+H]⁺=544.3.

### Step 4: Synthesis of 1-[3-(2,6-dimethoxyphenyl)-1H-pyrrolo[2,3-b]pyridin-6-yl]-3-[(3-fluoro-1-methylazetidin-3-yl)methyl]urea (Compound 187)

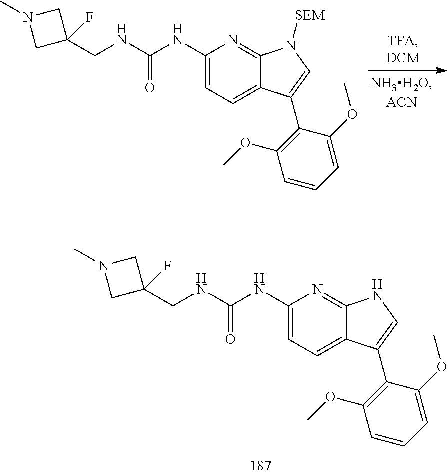 [SMILES: COc1cccc(OC)c1-c1cn(COCC[Si](C)(C)C)c2nc(NC(=O)NCC3(F)CN(C)C3)ccc12 ; COc1cccc(OC)c1-c1c[nH]c2nc(NC(=O)NCC3(F)CN(C)C3)ccc12]

To a solution of 1-[3-(2,6-dimethoxyphenyl)-1-{[2-(trimethylsilyl)ethoxy]methyl}pyrrolo[2,3-b]pyridin-6-yl]-3-[(3-fluoro-1-methylazetidin-3-yl)methyl]urea (400.0 mg, crude) in CH₂Cl₂ (20.0 mL) was added TFA (20.0 mL) at room temperature. The resulting mixture was stirred at room temperature for 4 h. The resulting mixture was concentrated under reduced pressure. To the residue in CH₃CN (20.0 mL) was added NH₃·H₂O (20.0 mL) at room temperature. The resulting mixture was stirred at room temperature for 16 h. After the reaction was completed, the resulting mixture was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 9 min; 254 nm) to afford 1-[3-(2,6-dimethoxyphenyl)-1H-pyrrolo[2,3-b]pyridin-6-yl]-3-[(3-fluoro-1-methylazetidin-3-yl)methyl]urea (Compound 187) (12.9 mg, 4%) as a white solid. LCMS (ESI, m/z): [M+H]⁺=414.2. ¹H NMR (400 MHz, DMSO-d₆): δ 11.40 (s, 1H), 9.25 (s, 1H), 8.76 (s, 1H), 7.49 (d, J=8.4 Hz, 1H), 7.28-7.24 (m, 1H), 7.17 (d, J=2.4 Hz, 1H), 6.97 (d, J=8.4 Hz, 1H), 6.73 (d, J=8.4 Hz, 2H), 3.75-3.67 (m, 10H), 3.46-3.31 (m, 2H), 2.45 (s, 3H).

### Example S179: Synthesis of cis-N-(3-(2,6-dimethoxyphenyl)-1H-pyrrolo[2,3-b]pyridin-6-yl)-2-fluoro-2-(hydroxymethyl)cyclopropanecarboxamide (Compound 188)

### Step 1: Synthesis of tert-butyl(2-fluoroallyloxy)diphenylsilane

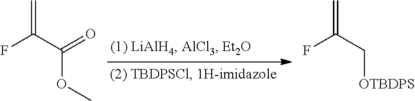

To a solution of LiAlH₄ (5.5 g, 114.12 mmol) in Et₂O (100.0 mL) was added AlCl₃ (6.4 g, 48.04 mmol) at 0° C. under N₂. The resulting mixture was stirred at 0° C. for 30 min. Then methyl 2-fluoroacrylate (5.0 g, 48.04 mmol) was added dropwise to the mixture at 0° C. The resulting mixture was stirred at room temperature for 1 h. After the reaction was completed, the resulting mixture was filtered. To the above filtrate was added TBDPSCl (19.8 g, 72.12 mmol) and 1H-imidazole (11.3 g, 96.15 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (95/5, v/v) to afford tert-butyl(2-fluoroallyloxy)diphenylsilane (4.0 g, 19%) as a yellow oil.

### Step 2: Synthesis of cis-ethyl 2-((tert-butyldiphenylsilyloxy)methyl)-2-fluorocyclopropanecarboxylate & trans-ethyl 2-((tert-butyldiphenylsilyloxy)methyl)-2-fluorocyclopropanecarboxylate

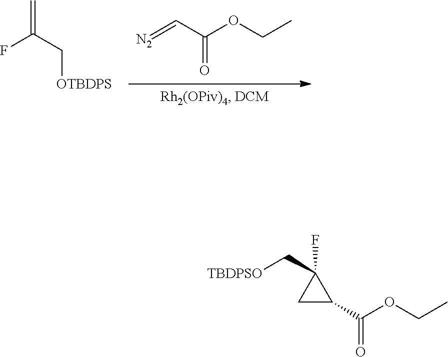

To a solution of tert-butyl(2-fluoroallyloxy)diphenylsilane (1.0 g, 3.18 mmol) in DCM (5.0 mL) was added dropwise ethyl 2-diazoacetate (1.1 g, 9.54 mmol) at 0° C. for 8 h. The resulting mixture was stirred at room temperature for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford cis-ethyl 2-((tert-butyldiphenylsilyloxy)methyl)-2-fluorocyclopropanecarboxylate (300.0 mg, 24%) as a yellow oil. LCMS (ESI, m/z): [M+H]⁺=401.2. and trans-ethyl 2-((tert-butyldiphenylsilyloxy)methyl)-2-fluorocyclopropanecarboxylate. (400.0 mg, 31%) as a yellow oil. LCMS (ESI, m/z): [M+H]⁺=401.2.

### Step 3: Synthesis of cis-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide

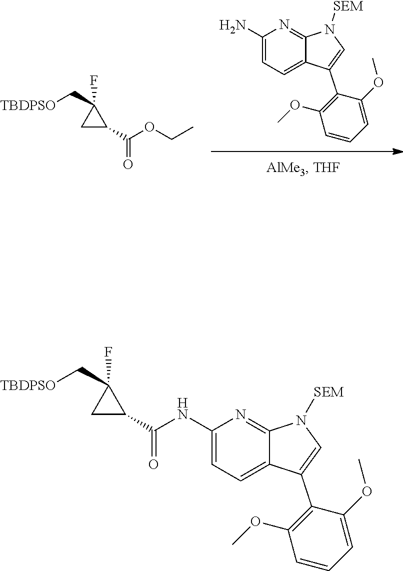

To a solution of 3-(2,6-dimethoxyphenyl)-1-((2-(trimethylsilyl)ethoxy)methyl)-1H-pyrrolo[2,3-b]pyridin-6-amine (400.0 mg, 1.00 mmol) in THE (10.0 mL) was added cis-ethyl 2-((tert-butyldiphenylsilyloxy)methyl)-2-fluorocyclopropanecarboxylate (1.2 g, 3.00 mmol) and AlMe₃ (2.5 mL, 2 mol/L) at 0° C. under N₂. The resulting mixture was stirred at 80° C. for 5 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (30/70, v/v) to afford cis-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide (150.0 mg, 20%) as a yellow solid. LCMS (ESI, m/z): [M+H]⁺=754.3.

### Step 4: Synthesis of cis-N-(3-(2,6-dimethoxyphenyl)-1H-pyrrolo[2,3-b]pyridin-6-yl)-2-fluoro-2-(hydroxymethyl)cyclopropanecarboxamide (Compound 188)

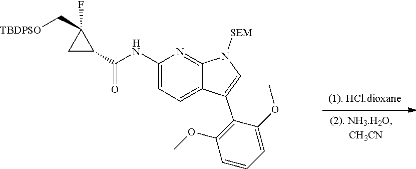

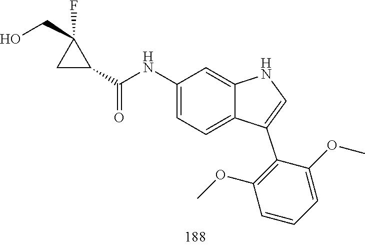 [SMILES: COc1cccc(OC)c1-c1c[nH]c2cc(NC(=O)[C@@H]3C[C@@]3(F)CO)ccc12]

The solution of cis-2-((tert-butyldiphenylsilyloxy)methyl)-N-(3-(2,6-dimethoxyphenyl)-1-((2-(trimethylsilyl)ethoxy)methyl)-1H-pyrrolo[2,3-b]pyridin-6-yl)-2-fluorocyclopropanecarboxamide (90.0 mg, 0.07 mmol) in HCl/dioxane (5.0 mL, 4 mol/L) was stirred at room temperature for 16 h. The resulting mixture was concentrated under reduced pressure. To the residue in ACN (5.0 mL) was added NH₃·H₂O (5.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse flash column chromatography with petroleum MeOH/H₂O (70/30, v/v) to afford cis-N-(3-(2,6-dimethoxyphenyl)-1H-pyrrolo[2,3-b]pyridin-6-yl)-2-fluoro-2-(hydroxymethyl)cyclopropanecarboxamide (Compound 188) (4.2 mg, 9%) as a white solid. LCMS (ESI, m/z): [M+H]⁺=386.2. ¹H NMR (400 MHz, DMSO-d₆): δ 11.41 (d, J=2.0 Hz, 1H), 10.40 (s, 1H), 7.81 ((d, J=8.4 Hz, 1H), 7.55 ((d, J=8.8 Hz, 1H), 7.30-7.26 (m, 2H), 6.75 (d, J=8.4 Hz, 2H), 5.13-5.10 (m, 1H), 3.73-3.66 (m, 8H), 2.34-2.29 (m, 1H), 1.77-1.70 (m, 1H), 1.29-1.08 (m, 1H).

### Example S180: Synthesis of (1S,2S)—N-[3-(4-cyclopropoxy-6-methoxypyrimidin-5-yl)-1H-pyrazolo[3,4-b]pyridin-6-yl]-2-fluorocyclopropane-1-carboxamide (Compound 189)

### Step 1: Synthesis of 4-cyclopropoxy-6-methoxy-5-(4,4,5,5-tetramethyl-1,3,2-dioxaborolan-2-yl)pyrimidine

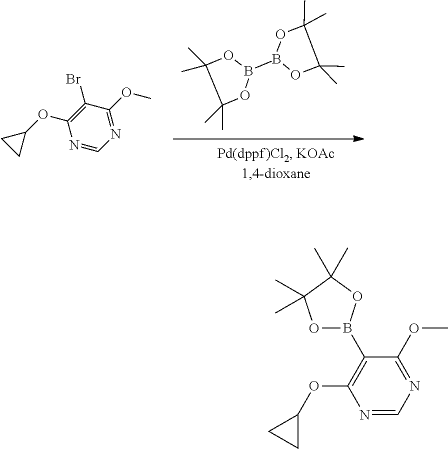

To a solution of 5-bromo-4-cyclopropoxy-6-methoxypyrimidine (500.0 mg, 2.04 mmol) in 1,4-dioxane (10.0 mL) was added 4,4,4′,4′,5,5,5′,5′-octamethyl-2,2′-bi(1,3,2-dioxaborolane) (1.6 g, 6.12 mmol), KOAc (400.5 mg, 4.08 mmol) and Pd(dppf)Cl₂ (149.3 mg, 0.20 mmol) at room temperature under N₂. The resulting mixture was stirred at 80° C. for 16 h. After the reaction was completed, the resulting mixture was concentrated under reduced pressure. The residue was purified by flash column chromatography with petroleum ether/ethyl acetate (3/1, v/v) to afford 4-cyclopropoxy-6-methoxy-5-(4,4,5,5-tetramethyl-1,3,2-dioxaborolan-2-yl)pyrimidine (240.0 mg, 40%) as a light brown oil. LCMS (ESI, m/z): [M+H]⁺=293.2.

### Step 2: Synthesis of 5-(6-chloro-1-{[2-(trimethylsilyl)ethoxy]methyl}pyrazolo[3,4-b]pyridin-3-yl)-4-cyclopropoxy-6-methoxypyrimidine

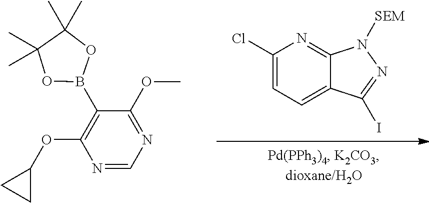 [SMILES: COc1ncnc(OC2CC2)c1B1OC(C)(C)C(C)(C)O1 ; C[Si](C)(C)CCOCn1nc(I)c2ccc(Cl)nc21]

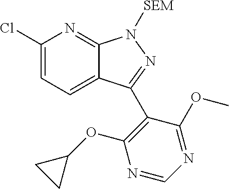 [SMILES: COc1ncnc(OC2CC2)c1-c1nn(COCC[Si](C)(C)C)c2nc(Cl)ccc12]

To a solution of 4-cyclopropoxy-6-methoxy-5-(4,4,5,5-tetramethyl-1,3,2-dioxaborolan-2-yl)pyrimidine (320.0 mg, 1.10 mmol) in 1,4-dioxane/H₂O (30.0 mL/6.0 mL) was add 6-chloro-3-iodo-1-{[2-(trimethylsilyl)ethoxy]methyl}pyrazolo[3,4-b]pyridine (372.5 mg, 0.91 mmol), K₂CO₃ (302.8 mg, 2.19 mmol) and Pd(PPh₃)₄ (126.6 mg, 0.11 mmol) at room temperature under N₂. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (7/1, v/v) to afford 5-(6-chloro-1-{[2-(trimethylsilyl)ethoxy]methyl}pyrazolo[3,4-b]pyridin-3-yl)-4-cyclopropoxy-6-methoxypyrimidine (295.0 mg, 60%) as a light yellow oil. LCMS (ESI, m/z): [M+H]⁺=448.1.

### Step 3: Synthesis of (1S,2S)—N-[3-(4-cyclopropoxy-6-methoxypyrimidin-5-yl)-1-{[2-(trimethylsilyl)ethoxy]methyl}pyrazolo[3,4-b]pyridin-6-yl]-2-fluorocyclopropane-1-carboxamide

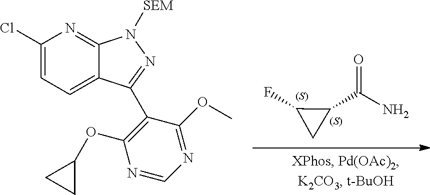

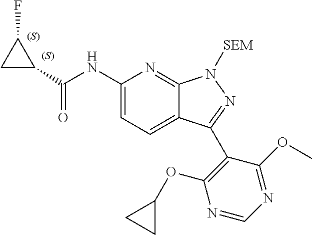

To a solution of 5-(6-chloro-1-{[2-(trimethylsilyl)ethoxy]methyl}pyrazolo[3,4-b]pyridin-3-yl)-4-cyclopropoxy-6-methoxypyrimidine (255.0 mg, 0.57 mmol) in t-BuOH (25.0 mL) was added (1S,2S)-2-fluorocyclopropane-1-carboxamide (293.4 mg, 2.85 mmol), XPhos (54.3 mg, 0.11 mmol), K₂CO₃ (236.0 mg, 1.71 mmol) and Pd(OAc)₂ (12.8 mg, 0.06 mmol) at room temperature under N₂. The final reaction mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/2, v/v) to afford (1S,2S)—N-[3-(4-cyclopropoxy-6-methoxypyrimidin-5-yl)-1-{[2-(trimethylsilyl)ethoxy]methyl}pyrazolo[3,4-b]pyridin-6-yl]-2-fluorocyclopropane-1-carboxamide (85.0 mg, 29%) as a colorless oil. LCMS (ESI, m/z): [M+H]⁺=515.2.

### Step 4: Synthesis of (1S,2S)—N-[3-(4-cyclopropoxy-6-methoxypyrimidin-5-yl)-1H-pyrazolo[3,4-b]pyridin-6-yl]-2-fluorocyclopropane-1-carboxamide (Compound 189)

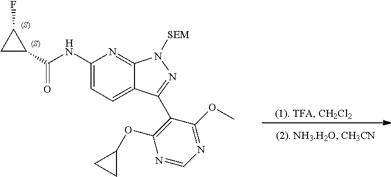

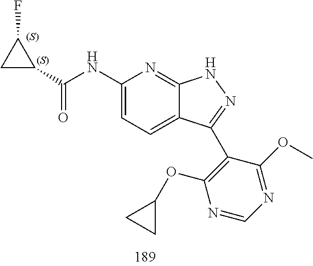

To a solution of (1S,2S)—N-[3-(4-cyclopropoxy-6-methoxypyrimidin-5-yl)-1-{[2-(trimethylsilyl)ethoxy]methyl}pyrazolo[3,4-b]pyridin-6-yl]-2-fluorocyclopropane-1-carboxamide (85.0 mg, 0.17 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (3.0 mL) was added NH₃·H₂O (3.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 25% B to 35% B in 9 min; 254/220 nm) to afford (1S,2S)—N-[3-(4-cyclopropoxy-6-methoxypyrimidin-5-yl)-1H-pyrazolo[3,4-b]pyridin-6-yl]-2-fluorocyclopropane-1-carboxamide (Compound 189) (13.5 mg, 21%) as a white solid. LCMS (ESI, m/z): [M+H]⁺=385.2. ¹H NMR (400 MHz, DMSO-d₆): δ 13.55 (s, 1H), 11.01 (s, 1H), 8.64 (s, 1H), 7.98-7.90 (m, 2H), 5.04-4.87 (m, 1H), 4.37-4.34 (m, 1H), 3.89 (s, 3H), 2.28-2.21 (m, 1H), 1.70-1.63 (m, 1H), 1.22-1.16 (m, 1H), 0.77-0.72 (m, 2H), 0.57-0.52 (m, 2H).

### Example S181: Synthesis of (1R,2R)—N-(3-(2,6-dimethoxyphenyl)-1H-pyrrolo[2,3-b]pyridin-6-yl)-2-((dimethylamino)methyl)cyclopropane-1-carboxamide and (1S,2S)—N-(3-(2,6-dimethoxyphenyl)-1H-pyrrolo[2,3-b]pyridin-6-yl)-2-((dimethylamino)methyl)cyclopropane-1-carboxamide (Compound 190 and Compound 191)

### Step 1: Synthesis of Trans-N-(3-(2,6-dimethoxyphenyl)-1-((2-(trimethylsilyl)ethoxy)methyl)-1H-pyrrolo[2,3-b]pyridin-6-yl)-2-((dimethylamino)methyl)cyclopropane-1-carboxamide

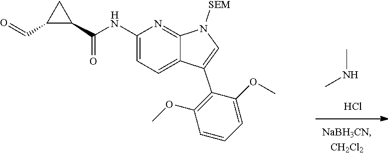

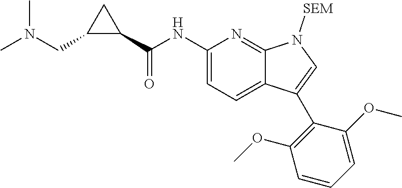

To a solution of trans-N-(3-(2,6-dimethoxyphenyl)-1-((2-(trimethylsilyl)ethoxy)methyl)-1H-pyrrolo[2,3-b]pyridin-6-yl)-2-formylcyclopropane-1-carboxamide (500.0 mg, 1.01 mmol) in CH₂Cl₂ (20.0 mL) was added dimethylamine hydrochloride (246.8 mg, 3.03 mmol) and NaBH₃CN (190.2 mg, 3.03 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with CH₂Cl₂/CH₃OH (80/20, v/v) to afford trans-N-(3-(2,6-dimethoxyphenyl)-1-((2-(trimethylsilyl)ethoxy)methyl)-1H-pyrrolo[2,3-b]pyridin-6-yl)-2-((dimethylamino)methyl)cyclopropane-1-carboxamide (210.0 mg, 39%) as a yellow solid. LCMS (ESI, m/z): [M+H]⁺=525.3.

### Step 2: Synthesis of (1R,2R)—N-(3-(2,6-dimethoxyphenyl)-1H-pyrrolo[2,3-b]pyridin-6-yl)-2-((dimethylamino)methyl)cyclopropane-1-carboxamide and (1S,2S)—N-(3-(2,6-dimethoxyphenyl)-1H-pyrrolo[2,3-b]pyridin-6-yl)-2-((dimethylamino)methyl)cyclopropane-1-carboxamide (Compound 190 and Compound 191)

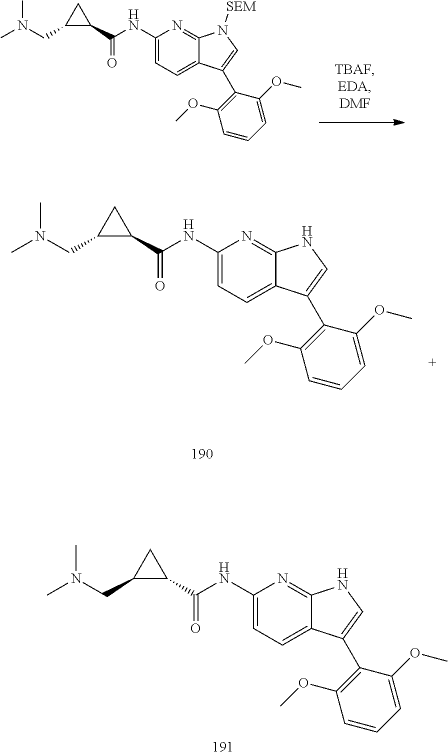

To a solution of trans-N-(3-(2,6-dimethoxyphenyl)-1-((2-(trimethylsilyl)ethoxy)methyl)-1H-pyrrolo[2,3-b]pyridin-6-yl)-2-((dimethylamino)methyl)cyclopropane-1-carboxamide (210.0 mg, 0.40 mmol) in DMF (5.0 mL) was added ethylenediamine (120.3 mg, 2.00 mmol) and TBAF (313.9 mg, 1.20 mmol) at room temperature. The resulting mixture was stirred at 80° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (75/25, v/v) and then separated by Prep-Chiral-HPLC with the following conditions (Column: CHIRALPAK ID, 2×25 cm, 5 um; Mobile Phase A: Hex (0.2% IPA)—HPLC, Mobile Phase B: EtOH—HPLC; Flow rate: 20 mL/min; Gradient: 30% to 30% in 23 min; 220/254 nm) to afford N-(3-(2,6-dimethoxyphenyl)-1H-pyrrolo[2,3-b]pyridin-6-yl)-2-((dimethylamino)methyl)cyclopropane-1-carboxamide Enantiomer 1 (19.8 mg, 12%) as a white solid and N-(3-(2,6-dimethoxyphenyl)-1H-pyrrolo[2,3-b]pyridin-6-yl)-2-((dimethylamino)methyl)cyclopropane-1-carboxamide Enantiomer 2 (33.3 mg, 21%) as a white solid. The absolute stereochemistry of Enantiomers 1 and 2 was not assigned. The two enantiomeric structures that could be obtained from chiral separation of the enantiomeric mixture as described above are shown as Compounds 190 and 191 in Table 1.

N-(3-(2,6-dimethoxyphenyl)-1H-pyrrolo[2,3-b]pyridin-6-yl)-2-((dimethylamino)methyl)cyclopropane-1-carboxamide Enantiomer 1: Retention Time 1:14.6 min; LCMS (ESI, m/z): [M+H]⁺=395.2. H NMR (300 MHz, DMSO-d₆): δ 11.42 (s, 1H), 10.52 (s, 1H), 7.81 (d, J=8.7 Hz, 1H), 7.53 (d, J=8.7 Hz, 1H), 7.30-7.25 (m, 2H), 6.75 (d, J=8.4 Hz, 2H), 3.69 (s, 6H), 2.32-2.25 (m, 1H), 2.19-2.12 (m, 7H), 1.92-1.86 (m, 1H), 1.36-1.32 (m, 1H), 1.07-1.04 (m, 1H), 0.74-0.65 (m, 1H).

N-(3-(2,6-dimethoxyphenyl)-1H-pyrrolo[2,3-b]pyridin-6-yl)-2-((dimethylamino)methyl)cyclopropane-1-carboxamide Enantiomer 2: Retention Time 2:18.357 min; LCMS (ESI, m/z): [M+H]⁺=395.2. H NMR (300 MHz, DMSO-d₆): δ 11.41 (s, 1H), 10.52 (s, 1H), 7.81 (d, J=8.7 Hz, 1H), 7.53 (d, J=8.4 Hz, 1H), 7.30-7.25 (m, 2H), 6.75 (d, J=8.4 Hz, 2H), 3.69 (s, 6H), 2.32-2.25 (m, 1H), 2.19-2.12 (m, 7H), 1.92-1.86 (m, 1H), 1.36-1.31 (m, 1H), 1.07-1.04 (m, 1H), 0.74-0.65 (m, 1H). d

### Example S182: Synthesis of trans-N-(3-(2,6-dimethoxyphenyl)-1H-pyrrolo[2,3-b]pyridin-6-yl)-3 (S)-fluoro-2-((4-methylpiperazin-1-yl)methyl)cyclopropane-1-carboxamide (Compound 192)

### Step 1: Synthesis of trans-(2-((benzyloxy)methyl)-3(R)-fluorocyclopropyl)methanol

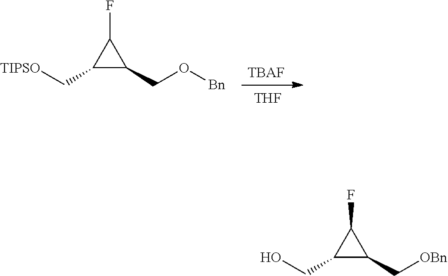

To a solution of (trans-(2-((benzyloxy)methyl)-3-fluorocyclopropyl)methoxy)triisopropylsilane (23.0 g, 62.74 mmol) in THE (200.0 mL) was added TBAF (19.7 g, 75.39 mmol) at 0° C. under N₂. The resulting mixture was stirred at 0° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (40/60, v/v) to afford trans-(2-((benzyloxy)methyl)-3(R)-fluorocyclopropyl)methanol (1.6 g, 12%) as a colorless oil. LCMS (ESI, m/z): [M+H]⁺=211.1.

### Step 2: Synthesis of trans-(2-((benzyloxy)methyl)-3(S)-fluorocyclopropyl)-1-carbaldehyde

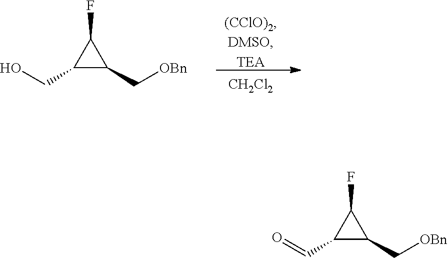

To a solution of (CClO)₂ (1.9 g, 15.22 mmol) in CH₂Cl₂ (20.0 mL) was added dropwise a solution of DMSO (4.8 g, 60.88 mmol) in CH₂Cl₂ (20.0 mL) at −78° C. under N₂. The resulting mixture was stirred at −78° C. for 0.5 h under N₂. Then a solution of trans-(2-((benzyloxy)methyl)-3(R)-fluorocyclopropyl)methanol (1.6 g, 7.61 mmol) in CH₂Cl₂ (20.0 mL) was added dropwise to the mixture at −78° C. under N₂. The resulting mixture was stirred at −78° C. for 1 h. Then TEA (6.2 g, 60.88 mmol) was added dropwise to the mixture at −78° C. under N₂. The resulting mixture was stirred at −78° C. for 0.5 h under N₂. After the reaction was completed, the resulting mixture was quenched with aqueous NH₄Cl at 0° C. and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford trans-(2-((benzyloxy)methyl)-3(S)-fluorocyclopropyl)-1-carbaldehyde (1.1 g, 69%) as a colorless oil. LCMS (ESI, m/z): [M+H]⁺=209.1.

### Step 3: Synthesis of trans-(2-((benzyloxy)methyl)-3(S)-fluorocyclopropyl)-1-carboxylic acid

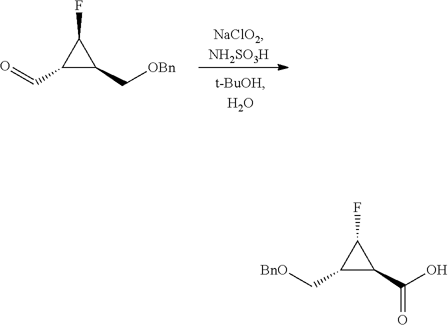 [SMILES: O=C[C@@H]1[C@@H](F)[C@H]1COCc1ccccc1 ; O=C(O)[C@@H]1[C@@H](F)[C@H]1COCc1ccccc1]

To a mixture of trans-(2-((benzyloxy)methyl)-3(S)-fluorocyclopropyl)-1-carbaldehyde (1.1 g, 5.28 mmol) and NH₂SO₃H (5.1 g, 52.83 mmol) in t-BuOH/H₂O (2.0/18.0 mL) was added NaClO₂ (4.8 g, 52.83 mmol) at 0° C. under N₂. The resulting mixture was stirred at room temperature for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (50/50, v/v) to afford trans-(2-((benzyloxy)methyl)-3(S)-fluorocyclopropyl)-1-carboxylic acid (800.0 mg, 67%) as a colorless oil. LCMS (ESI, m/z): [M+H]⁺=225.1.

### Step 4: Synthesis of trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(S)-fluorocyclopropane-1-carboxamide

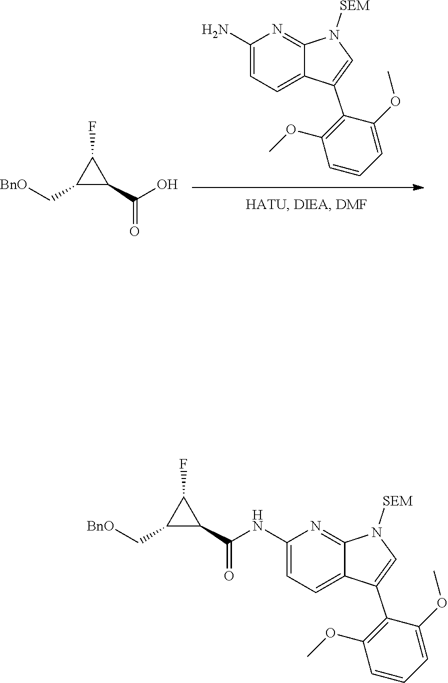 [SMILES: O=C(O)[C@@H]1[C@@H](F)[C@H]1COCc1ccccc1 ; COc1cccc(OC)c1-c1cn(COCC[Si](C)(C)C)c2nc(N)ccc12 ; COc1cccc(OC)c1-c1cn(COCC[Si](C)(C)C)c2nc(NC(=O)[C@@H]3[C@@H](F)[C@H]3COCc3ccccc3)ccc12]

To a mixture of 3-(2,6-dimethoxyphenyl)-1-((2-(trimethylsilyl)ethoxy)methyl)-1H-pyrrolo[2,3-b]pyridin-6-amine (1.4 g, 3.57 mmol) and DIEA (1.4 g, 10.70 mmol) in DMF (20.0 mL) was added trans-(2-((benzyloxy)methyl)-3(S)-fluorocyclopropyl)-1-carboxylic acid (800.0 mg, 3.57 mmol) and HATU (1.6 g, 4.28 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (90/10, v/v) to afford trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(S)-fluorocyclopropane-1-carboxamide (1.4 g, 65%) as a white solid. LCMS (ESI, m/z): [M+H]⁺=606.3.

### Step 5: Synthesis of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-(hydroxymethyl)cyclopropane-1-carboxamide

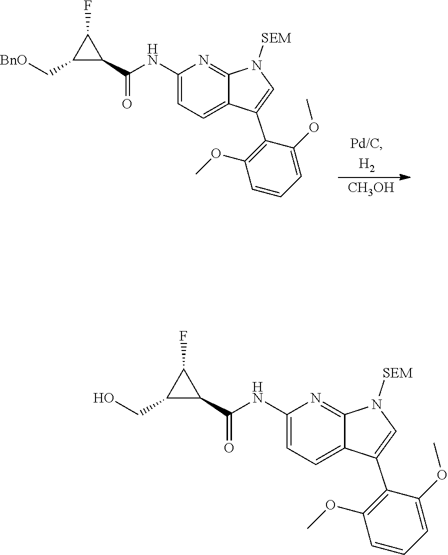

To a solution of trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(S)-fluorocyclopropane-1-carboxamide (1.3 g, 2.15 mmol) in CH₃OH (15.0 mL) was added Pd/C (228.4 mg, dry) at room temperature under N₂. The resulting mixture was stirred at room temperature for 4 h under H₂. After the reaction was completed, the resulting mixture was filtered. The filtrate was concentrated under reduced pressure. The residue was purified by reverse phase flash column chromatography with petroleum CH₃CN/H₂O (28/72, v/v) to afford trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-(hydroxymethyl)cyclopropane-1-carboxamide (570.0 mg, 25%) as a white solid. LCMS (ESI, m/z): [M+H]⁺=516.2.

### Step 6: Synthesis of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-formylcyclopropane-1-carboxamide

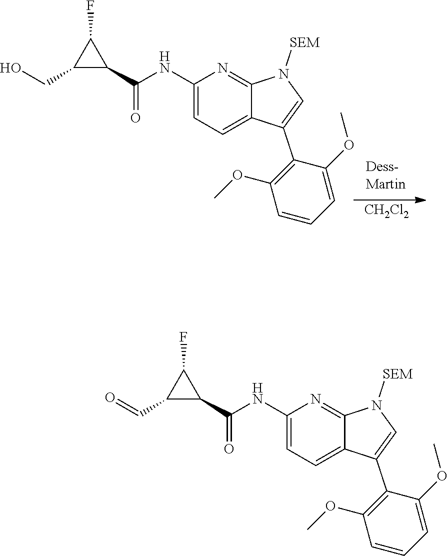

To a solution of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-(hydroxymethyl)cyclopropane-1-carboxamide (570.0 mg, 1.11 mmol) in CH₂Cl₂ (10.0 mL) was added Dess-Martin (701.1 mg, 1.62 mmol) at room temperature. The resulting mixture was stirred at room temperature for 2 h. After the reaction was completed, the resulting mixture was diluted with H₂O and extracted with CH₂Cl₂. The combined organic layer was washed with aq. NaHCO₃, brine, dried over anhydrous sodium sulfate and filtered. The filtrate was concentrated under reduced pressure to afford trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-formylcyclopropane-1-carboxamide (600.0 mg, crude) as a brown solid. LCMS (ESI, m/z): [M+H]⁺=514.2.

### Step 7: Synthesis of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-((4-methylpiperazin-1-yl)methyl)cyclopropane-1-carboxamide

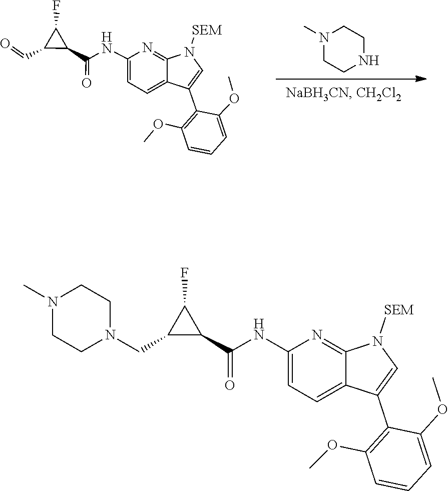

To a mixture of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-formylcyclopropane-1-carboxamide (600.0 mg, crude) and 1-methylpiperazine (351.0 mg, 3.51 mmol) in CH₂C₂ (10.0 mL) was added NaBH₃CN (330.3 mg, 3.51 mmol) at room temperature. The resulting mixture was stirred at room temperature for 4 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by Prep-TLC with CH₂Cl₂/CH₃OH (20/1, v/v) to afford trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-((4-methylpiperazin-1-yl)methyl)cyclopropane-1-carboxamide (200.0 mg, 34%) as a brown solid. LCMS (ESI, m/z): [M+H]⁺=598.3.

### Step 8: Synthesis of trans-N-(3-(2,6-dimethoxyphenyl)-1H-pyrrolo[2,3-b]pyridin-6-yl)-3(S)-fluoro-2-((4-methylpiperazin-1-yl)methyl)cyclopropane-1-carboxamide (Compound 192)

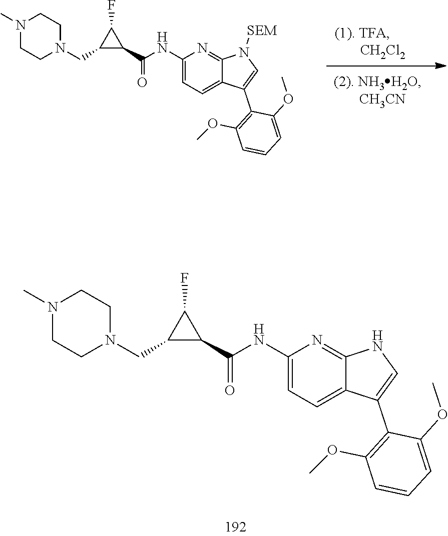 [SMILES: COc1cccc(OC)c1-c1cn(COCC[Si](C)(C)C)c2nc(NC(=O)[C@@H]3[C@@H](F)[C@H]3CN3CCN(C)CC3)ccc12 ; COc1cccc(OC)c1-c1c[nH]c2nc(NC(=O)[C@@H]3[C@@H](F)[C@H]3CN3CCN(C)CC3)ccc12]

To a solution of trans-N-(3-(2,6-dimethoxyphenyl)-1-((2-(trimethylsilyl)ethoxy)methyl)-1H-pyrrolo[2,3-b]pyridin-6-yl)-3(S)-fluoro-2-((4-methylpiperazin-1-yl)methyl)cyclopropane-1-carboxamide (180.0 mg, 0.30 mmol) in CH₂Cl₂ (4.0 mL) was added TFA (4.0 mL) at room temperature. The resulting mixture was stirred at room temperature for 2 h. The resulting mixture was concentrated under reduced pressure. To the residue in CH₃CN (4.0 mL) was added NH₃·H₂O (4.0 mL) at room temperature. The resulting mixture was stirred at room temperature for 8 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 30×150 mm, 5 m; Mobile Phase A: Water (10 mmol/L NH₄HCO₃), Mobile Phase B: ACN; Flow rate: 60 mL/min; Gradient: 25% B to 50% B in 8 min; Wave Length: 254 nm) to afford trans-N-(3-(2,6-dimethoxyphenyl)-1H-pyrrolo[2,3-b]pyridin-6-yl)-3(S)-fluoro-2-((4-methylpiperazin-1-yl)methyl)cyclopropane-1-carboxamide (Compound 192) (5.2 mg, 4%) as a white solid. LCMS (ESI, m/z): [M+H]⁺=468.2. ¹H NMR (400 MHz, DMSO-d₆): δ 11.45 (s, 1H), 10.63 (s, 1H), 7.75 (d, J=8.4 Hz, 1H), 7.54 (d, J=8.8 Hz, 1H), 7.30-7.26 (m, 2H), 6.75 (d, J=8.4 Hz, 2H), 4.99-4.82 (m, 1H), 3.69 (s, 6H), 2.73-2.66 (m, 1H), 2.62-2.50 (m, 1H), 2.47-2.26 (m, 8H), 2.09 (s, 3H), 2.06-1.99 (m, 1H).

### Example S183: Synthesis of 1-[3-(2,6-dimethoxyphenyl)-1H-pyrrolo[2,3-b]pyridin-6-yl]-3-(2-{6-methyl-3,6-diazabicyclo[3.1.1]heptan-3-yl}ethyl)urea (Compound 193)

### Step 1: Synthesis of Phenyl (3-(2,6-dimethoxyphenyl)-1-((2-(trimethylsilyl)ethoxy)methyl)-1H-pyrrolo[2,3-b]pyridin-6-yl)carbamate

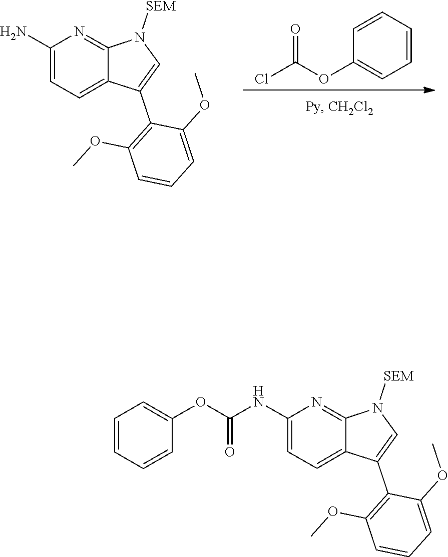 [SMILES: COc1cccc(OC)c1-c1cn(COCC[Si](C)(C)C)c2nc(N)ccc12 ; O=C(Cl)Oc1ccccc1 ; COc1cccc(OC)c1-c1cn(COCC[Si](C)(C)C)c2nc(NC(=O)Oc3ccccc3)ccc12]

To a solution of 3-(2,6-dimethoxyphenyl)-1-((2-(trimethylsilyl)ethoxy)methyl)-1H-pyrrolo[2,3-b]pyridin-6-amine (300.0 mg, 0.75 mmol) in CH₂Cl₂ (5.0 mL) was added pyridine (224.0 mg, 3.00 mmol) and phenyl chloroformate (140.4 mg, 0.90 mmol) at 0° C. under N₂. The resulting mixture was stirred at 0° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was evaporated in vacuo to afford phenyl (3-(2,6-dimethoxyphenyl)-1-((2-(trimethylsilyl)ethoxy)methyl)-1H-pyrrolo[2,3-b]pyridin-6-yl)carbamate (400.0 mg, crude) as a colorless oil. LCMS (ESI, m/z): [M+H]⁺=520.2

### Step 2: Synthesis of 1-[3-(2,6-dimethoxyphenyl)-1-{[2-(trimethylsilyl)ethoxy]methyl}pyrrolo[2,3-b]pyridin-6-yl]-3-(2-hydroxyethyl)urea

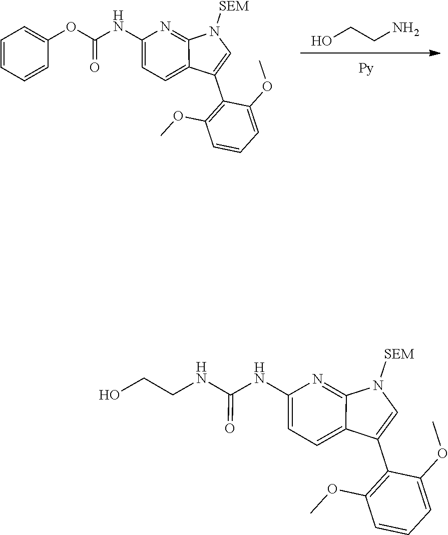

To a solution of phenyl N-[3-(2,6-dimethoxyphenyl)-1-{[2-(trimethylsilyl)ethoxy]methyl}pyrrolo[2,3-b]pyridin-6-yl]carbamate (300.0 mg, 0.57 mmol) in pyridine (3.0 mL) was added ethanolamine (42.3 mg, 0.69 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (92/8, v/v) to afford 1-[3-(2,6-dimethoxyphenyl)-1-{[2-(trimethylsilyl)ethoxy]methyl}pyrrolo[2,3-b]pyridin-6-yl]-3-(2-hydroxyethyl)urea (250.0 mg, 78%) as a white solid. LCMS (ESI, m/z): [M+H]⁺=487.2

### Step 3: Synthesis of 1-[3-(2,6-dimethoxyphenyl)-1-{[2-(trimethylsilyl)ethoxy]methyl}pyrrolo[2,3-b]pyridin-6-yl]-3-(2-oxoethyl)urea

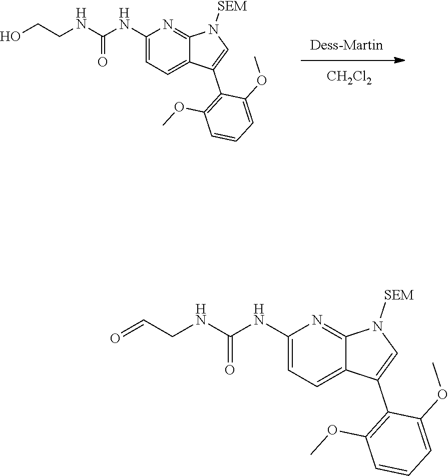 [SMILES: COc1cccc(OC)c1-c1cn(COCC[Si](C)(C)C)c2nc(NC(=O)NCCO)ccc12 ; "CH2Cl2" ; COc1cccc(OC)c1-c1cn(COCC[Si](C)(C)C)c2nc(NC(=O)NCC=O)ccc12]

To a solution of 1-[3-(2,6-dimethoxyphenyl)-1-{[2-(trimethylsilyl)ethoxy]methyl}pyrrolo[2,3-b]pyridin-6-yl]-3-(2-hydroxyethyl)urea (100.0 mg, 0.21 mmol) in CH₂Cl₂ (3.0 mL) was added Dess-Martin (348.6 mg, 0.82 mmol) at room temperature under N₂. The resulting mixture was stirred at room temperature for 4 h under N₂. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure to afford 1-[3-(2,6-dimethoxyphenyl)-1-{[2-(trimethylsilyl)ethoxy]methyl}pyrrolo[2,3-b]pyridin-6-yl]-3-(2-oxoethyl)urea (100.0 mg, crude) as a black solid. LCMS (ESI, m/z): [M+H]⁺=485.2

### Step 4: Synthesis of 1-[3-(2,6-dimethoxyphenyl)-1-{[2-(trimethylsilyl)ethoxy]methyl}pyrrolo[2,3-b]pyridin-6-yl]-3-(2-{6-methyl-3,6-diazabicyclo[3.1.1]heptan-3-yl}ethyl)urea

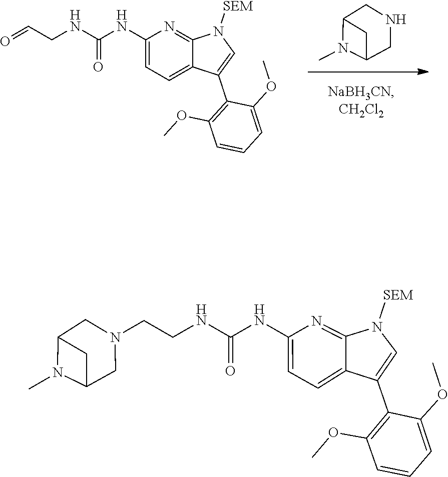

To a solution of 1-[3-(2,6-dimethoxyphenyl)-1-{[2-(trimethylsilyl)ethoxy]methyl}pyrrolo[2,3-b]pyridin-6-yl]-3-(2-oxoethyl)urea (100.0 mg, crude) in CH₂Cl₂ (3.0 mL) was added 6-methyl-3,6-diazabicyclo[3.1.1]heptane (69.4 mg, 0.61 mmol) and NaBH₃CN (13.0 mg, 0.20 mmol) at room temperature. The resulting mixture was stirred at room temperature for 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with petroleum CH₃CN/H₂O (60/40, v/v) to afford 1-[3-(2,6-dimethoxyphenyl)-1-{[2-(trimethylsilyl)ethoxy]methyl}pyrrolo[2,3-b]pyridin-6-yl]-3-(2-{6-methyl-3,6-diazabicyclo[3.1.1]heptan-3-yl}ethyl)urea (30.0 mg, 25%) as a white solid. LCMS (ESI, m/z): [M+H]⁺=581.3.

### Step 5: Synthesis of 1-[3-(2,6-dimethoxyphenyl)-1H-pyrrolo[2,3-b]pyridin-6-yl]-3-(2-{6-methyl-3,6-diazabicyclo[3.1.1]heptan-3-yl}ethyl)urea (Compound 193)

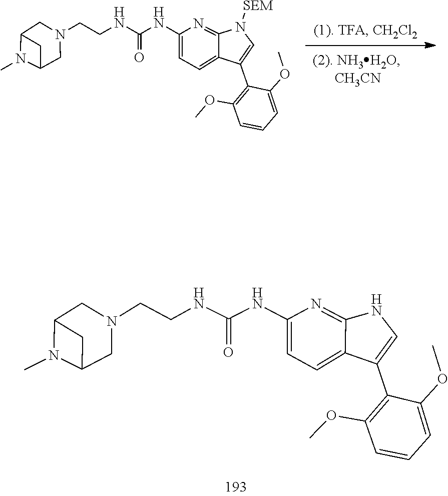 [SMILES: COc1cccc(OC)c1-c1cn(COCC[Si](C)(C)C)c2nc(NC(=O)NCCN3CC4CC(C3)N4C)ccc12 ; COc1cccc(OC)c1-c1c[nH]c2nc(NC(=O)NCCN3CC4CC(C3)N4C)ccc12]

To a solution of 1-[3-(2,6-dimethoxyphenyl)-1-{[2-(trimethylsilyl)ethoxy]methyl}pyrrolo[2,3-b]pyridin-6-yl]-3-(2-{6-methyl-3,6-diazabicyclo[3.1.1]heptan-3-yl}ethyl)urea (30.0 mg, 0.06 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (3.0 mL) was added NH₃·H₂O (3.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 9 min; 254 nm) to afford 1-[3-(2,6-dimethoxyphenyl)-1H-pyrrolo[2,3-b]pyridin-6-yl]-3-(2-{6-methyl-3,6-diazabicyclo[3.1.1]heptan-3-yl}ethyl)urea (Compound 193) (1.4 mg, 6%) as a white solid. LCMS (ESI, m/z): [M+H]⁺=451.4. H NMR (400 MHz, DMSO-d₆): δ 11.27 (s, 1H), 9.17 (s, 1H), 8.49 (s, 1H), 7.46 (d, J=8.4 Hz, 1H), 7.27 (d, J=8.4 Hz, 1H), 7.15 (d, J=2.0 Hz, 1H), 6.97 (d, J=8.4 Hz, 1H), 6.73 (d, J=8.4 Hz, 2H), 3.68 (s, 6H), 2.93-2.83 (m, 4H), 2.76-2.73 (m, 2H), 2.26-2.12 (m, 1H), 2.01 (s, 3H), 1.87-1.82 (m, 1H).

### Example S184: Synthesis of 3-[3-(2,4-dimethoxypyridin-3-yl)-1H-pyrrolo[2,3-b]pyridin-6-yl]-1-[2-(4-ethylpiperazin-1-yl)ethyl]urea (Compound 194)

### Step 1: Synthesis of 6-chloro-3-(2,4-dimethoxypyridin-3-yl)-1-((2-(trimethylsilyl)ethoxy)methyl)-1H-pyrrolo[2,3-b]pyridine

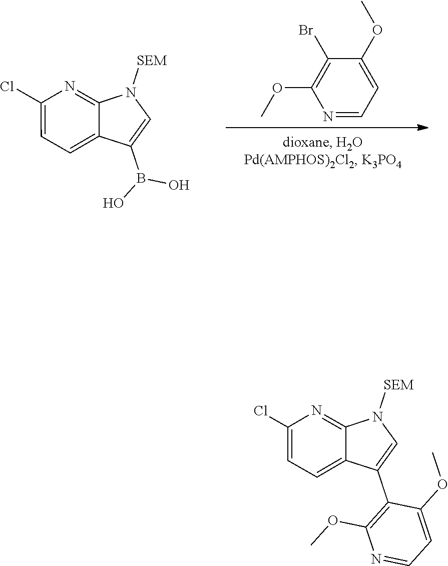 [SMILES: C[Si](C)(C)CCOCn1cc(B(O)O)c2ccc(Cl)nc21 ; COc1ccnc(OC)c1Br ; COc1ccnc(OC)c1-c1cn(COCC[Si](C)(C)C)c2nc(Cl)ccc12]

To a solution of 6-chloro-1-[[2-(trimethylsilyl)ethoxy]methyl]pyrrolo[2,3-b]pyridin-3-ylboronic acid (1.20 g, 3.67 mmol) in H₂O (1.5 mL) and dioxane (15.0 mL) was added 3-bromo-2,4-dimethoxypyridine (961.2 mg, 4.48 mmol), Pd(AMPHOS)₂C₁₂ (260.1 mg, 0.37 mmol) and K₃PO₄ (2.4 g, 11.24 mmol). The resulting mixture was stirred at 80° C. for 12 h under N₂. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 6-chloro-3-(2,4-dimethoxypyridin-3-yl)-1-((2-(trimethylsilyl)ethoxy)methyl)-1H-pyrrolo[2,3-b]pyridine (830.0 mg, 54%) as a red oil. LCMS (ESI, m/z): [M+H]⁺=420.1.

### Step 2: Synthesis of N-(3-(2,4-dimethoxypyridin-3-yl)-1-((2-(trimethylsilyl)ethoxy)methyl)-1H-pyrrolo[2,3-b]pyridin-6-yl)-1,1-diphenylmethanimine

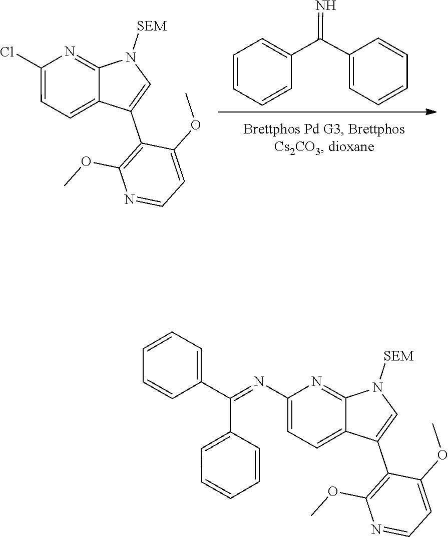 [SMILES: COc1ccnc(OC)c1-c1cn(COCC[Si](C)(C)C)c2nc(Cl)ccc12 ; N=C(c1ccccc1)c1ccccc1 ; COc1ccnc(OC)c1-c1cn(COCC[Si](C)(C)C)c2nc(N=C(c3ccccc3)c3ccccc3)ccc12]

To a solution of 6-chloro-3-(2,4-dimethoxypyridin-3-yl)-1-((2-(trimethylsilyl)ethoxy)methyl)-1H-pyrrolo[2,3-b]pyridine (830.0 mg, 1.98 mmol) in dioxane (10.0 mL) was added diphenylmethanimine (1.0 g, 5.94 mmol), Brettphos Pd G3 (194.5 mg, 0.20 mmol), Brettphos (216.4 mg, 0.40 mmol) and Cs₂CO₃ (1936.4 mg, 5.94 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/2, v/v) to afford N-(3-(2,4-dimethoxypyridin-3-yl)-1-((2-(trimethylsilyl)ethoxy)methyl)-1H-pyrrolo[2,3-b]pyridin-6-yl)-1,1-diphenylmethanimine (1.0 g, 89%) as a light red oil. LCMS (ESI, m/z): [M+H]⁺=565.3.

### Step 3: Synthesis of 3-(2,4-dimethoxypyridin-3-yl)-1-((2-(trimethylsilyl)ethoxy)methyl)-1H-pyrrolo[2,3-b]pyridin-6-amine

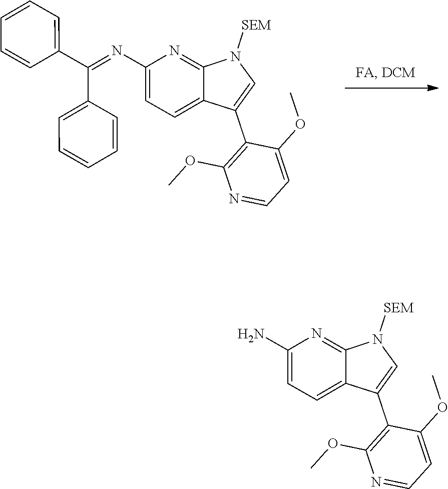 [SMILES: COc1ccnc(OC)c1-c1cn(COCC[Si](C)(C)C)c2nc(N=C(c3ccccc3)c3ccccc3)ccc12 ; COc1ccnc(OC)c1-c1cn(COCC[Si](C)(C)C)c2nc(N)ccc12]

To a solution of N-(3-(2,4-dimethoxypyridin-3-yl)-1-((2-(trimethylsilyl)ethoxy)methyl)-1H-pyrrolo[2,3-b]pyridin-6-yl)-1,1-diphenylmethanimine (1.0 g, 0.47 mmol) in DCM (10.0 mL) was added FA (5.0 mL) at room temperature. The mixture was stirred at room temperature for 2 h. After the reaction was completed, the resulting mixture was diluted with H₂O. The pH value of the mixture was adjusted to 7 with NaHCO₃ solution. The mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 3-(2,4-dimethoxypyridin-3-yl)-1-((2-(trimethylsilyl)ethoxy)methyl)-1H-pyrrolo[2,3-b]pyridin-6-amine (530.0 mg, 75%) as a red oil. LCMS (ESI, m/z): [M+H]⁺=401.2.

### Step 4: Synthesis of 3-[3-(2,4-dimethoxypyridin-3-yl)-1-[[2-(trimethylsilyl)ethoxy]methyl]pyrrolo[2,3-b]pyridin-6-yl]-1-[2-(4-ethylpiperazin-1-yl)ethyl]urea

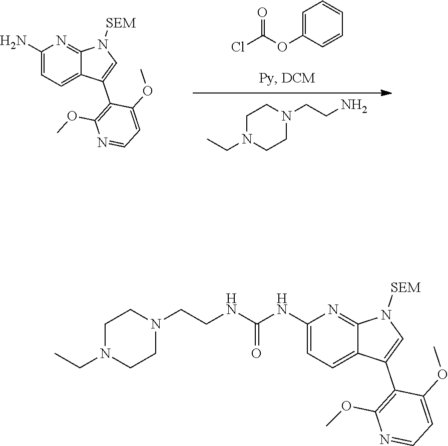 [SMILES: COc1ccnc(OC)c1-c1cn(COCC[Si](C)(C)C)c2nc(N)ccc12 ; CCN1CCN(CCN)CC1 ; O=C(Cl)Oc1ccccc1 ; CCN1CCN(CCNC(=O)Nc2ccc3c(-c4c(OC)ccnc4OC)cn(COCC[Si](C)(C)C)c3n2)CC1]

To a solution of 3-(2,4-dimethoxypyridin-3-yl)-1-[[2-(trimethylsilyl)ethoxy]methyl]pyrrolo[2,3-b]pyridin-6-amine (200.0 mg, 0.50 mmol) in pyridine/DCM (10.0 mL/5.0 mL) was added phenyl chloroformate (78.18 mg, 0.499 mmol) at 0° C. under N₂. The mixture was stirred at 0° C. for 1 h. The resulting mixture was concentrated under vacuum. To the above mixture was added a solution of 2-(4-ethylpiperazin-1-yl)ethan-1-amine (235.7 mg, 1.49 mmol) in pyridine (10.0 mL). The resulting mixture was stirred at 60° C. for 2 h under N₂. After the reaction was completed, the resulting mixture was concentrated under vacuum. The residue was purified by reverse phase flash column chromatography with CH₃CN/H₂O (9/1, v/v) to afford 3-[3-(2,4-dimethoxypyridin-3-yl)-1-[[2-(trimethylsilyl)ethoxy]methyl]pyrrolo[2,3-b]pyridin-6-yl]-1-[2-(4-ethylpiperazin-1-yl)ethyl]urea (90.0 mg, 31%) as an off-white solid. LCMS (ESI, m/z): [M+H]⁺=584.3.

### Step 5: Synthesis of 3-[3-(2,4-dimethoxypyridin-3-yl)-1H-pyrrolo[2,3-b]pyridin-6-yl]-1-[2-(4-ethylpiperazin-1-yl)ethyl]urea (Compound 194)

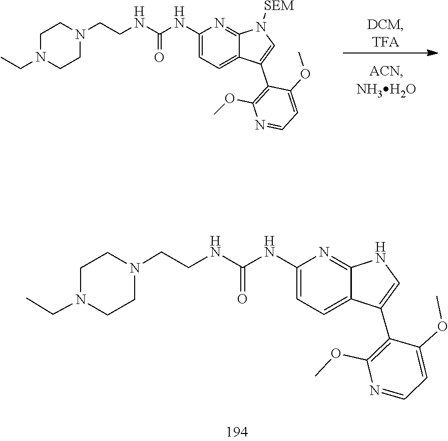 [SMILES: CCN1CCN(CCNC(=O)Nc2ccc3c(-c4c(OC)ccnc4OC)cn(COCC[Si](C)(C)C)c3n2)CC1 ; CCN1CCN(CCNC(=O)Nc2ccc3c(-c4c(OC)ccnc4OC)c[nH]c3n2)CC1]

To a solution of 3-[3-(2,4-dimethoxypyridin-3-yl)-1-[[2-(trimethylsilyl)ethoxy]methyl]pyrrolo[2,3-b]pyridin-6-yl]-1-[2-(4-ethylpiperazin-1-yl)ethyl]urea (70.0 mg, 0.12 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 10% B to 40% B in 9 min; 254 nm) to afford 3-[3-(2,4-dimethoxypyridin-3-yl)-1H-pyrrolo[2,3-b]pyridin-6-yl]-1-[2-(4-ethylpiperazin-1-yl)ethyl]urea (Compound 194) (6.8 mg, 13%) as a white solid. LCMS (ESI, m/z): [M+H]⁺=454.3. ¹H NMR (400 MHz, DMSO-d₆): δ 11.40 (s, 1H), 9.16 (s, 1H), 8.35 (s, 1H), 8.05 (d, J=5.6 Hz, 1H), 7.52 (d, J=8.8 Hz, 1H), 7.25 (d, J=2.4 Hz, 1H), 7.03 (d, J=8.4 Hz, 1H), 6.87 (d, J=6.0 Hz, 1H), 3.80 (s, 3H), 3.79 (s, 3H), 2.52-2.44 (m, 5H), 2.36-2.26 (m, 5H), 0.99-0.96 (m, 3H).

### Example S185: Synthesis of 1-(3-(2,4-dimethoxypyridin-3-yl)-1H-pyrrolo[2,3-b]pyridin-6-yl)-3-(3-(dimethylamino)propyl)urea (Compound 195)

### Step 1: Synthesis of 6-chloro-3-(2,4-dimethoxypyridin-3-yl)-1-((2-(trimethylsilyl)ethoxy)methyl)-1H-pyrrolo[2,3-b]pyridine

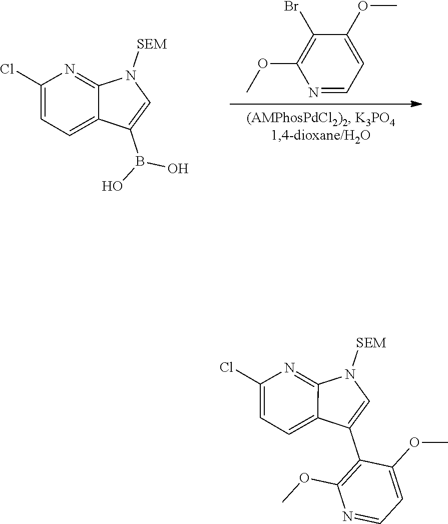

To a solution of 3-bromo-2,4-dimethoxypyridine (380.0 mg, 1.74 mmol) in 1,4-dioxane/H₂O (10.0 mL/2.0 mL) was added (6-chloro-1-((2-(trimethylsilyl)ethoxy)methyl)-1H-pyrrolo[2,3-b]pyridin-3-yl)boronic acid (683.1 mg, 2.09 mmol), K₃PO₄ (1109.8 mg, 5.23 mmol) and (AMPhosPdCl₂)₂ (123.4 mg, 0.17 mmol) at room temperature under N₂. The resulting mixture was stirred at 80° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford 6-chloro-3-(2,4-dimethoxypyridin-3-yl)-1-((2-(trimethylsilyl)ethoxy)methyl)-1H-pyrrolo[2,3-b]pyridine (220.0 mg, 30%) as a yellow solid. LCMS (ESI, m/z): [M+H]⁺=420.1.

### Step 2: Synthesis of Tert-butyl (3-(2,4-dimethoxypyridin-3-yl)-1-((2-(trimethylsilyl)ethoxy)methyl)-1H-pyrrolo[2,3-b]pyridin-6-yl)carbamate

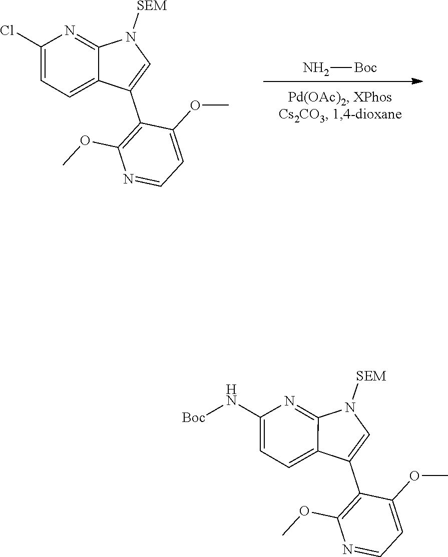 [SMILES: COc1ccnc(OC)c1-c1cn(COCC[Si](C)(C)C)c2nc(Cl)ccc12 ; CC(C)(C)OC(N)=O ; COc1ccnc(OC)c1-c1cn(COCC[Si](C)(C)C)c2nc(NC(=O)OC(C)(C)C)ccc12]

To a solution of 6-chloro-3-(2,4-dimethoxypyridin-3-yl)-1-((2-(trimethylsilyl)ethoxy)methyl)-1H-pyrrolo[2,3-b]pyridine (180.0 mg, 0.43 mmol) in 1,4-dioxane (10.0 mL) was added NH₂-Boc (65.3 mg, 0.56 mmol), Cs₂CO₃ (418.9 mg, 1.29 mmol), XPhos (40.9 mg, 0.09 mmol) and Pd(OAc)₂ (9.6 mg, 0.04 mmol) at room temperature under N₂. The resulting mixture was stirred at 100° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (50/50, v/v) to afford tert-butyl (3-(2,4-dimethoxypyridin-3-yl)-1-((2-(trimethylsilyl)ethoxy)methyl)-1H-pyrrolo[2,3-b]pyridin-6-yl)carbamate (200.0 mg, 93%) as a yellow solid. LCMS (ESI, m/z): [M+H]⁺=501.2.

### Step 3: Synthesis of 3-(2,4-dimethoxypyridin-3-yl)-1-((2-(trimethylsilyl)ethoxy)methyl)-1H-pyrrolo[2,3-b]pyridin-6-amine

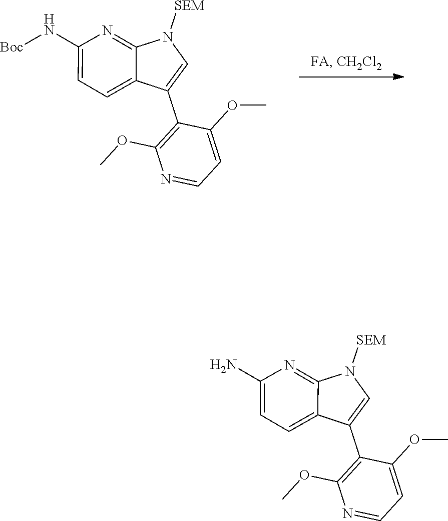 [SMILES: COc1ccnc(OC)c1-c1cn(COCC[Si](C)(C)C)c2nc(NC(=O)OC(C)(C)C)ccc12 ; COc1ccnc(OC)c1-c1cn(COCC[Si](C)(C)C)c2nc(N)ccc12]

To a solution of tert-butyl (3-(2,4-dimethoxypyridin-3-yl)-1-((2-(trimethylsilyl)ethoxy)methyl)-1H-pyrrolo[2,3-b]pyridin-6-yl)carbamate (180.0 mg, 0.45 mmol) in CH₂Cl₂ (10.0 mL) was added FA (15.0 mL) at room temperature. The resulting mixture was stirred at room temperature for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 3-(2,4-dimethoxypyridin-3-yl)-1-((2-(trimethylsilyl)ethoxy)methyl)-1H-pyrrolo[2,3-b]pyridin-6-amine (140.0 mg, crude) as a yellow solid. LCMS (ESI, m/z): [M+H]⁺=401.2.

### Step 4: Synthesis of 1-(3-(2,4-dimethoxypyridin-3-yl)-1-((2-(trimethylsilyl)ethoxy)methyl)-1H-pyrrolo[2,3-b]pyridin-6-yl)-3-(3-(dimethylamino)propyl)urea

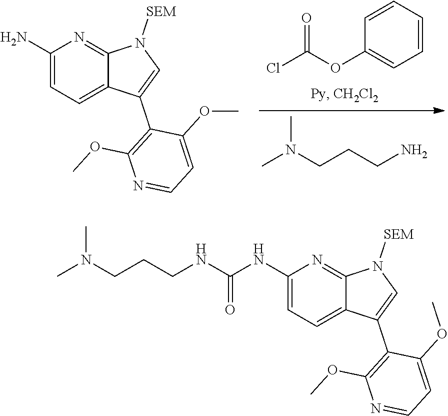

To a solution of 3-(2,4-dimethoxypyridin-3-yl)-1-((2-(trimethylsilyl)ethoxy)methyl)-1H-pyrrolo[2,3-b]pyridin-6-amine (100.0 mg, 0.25 mmol) in CH₂Cl₂ (10.0 mL) was added phenyl carbonochloridate (117.3 mg, 0.75 mmol) and pyridine (80.0 mg, 1.00 mmol) at room temperature under N₂. The resulting mixture was stirred at room temperature for 3 h. The resulting mixture was concentrated under reduced pressure. The residue was dissolved in pyridine (10.0 mL). Then N¹, N¹-dimethylpropane-1,3-diamine (30.6 mg, 0.30 mmol) was added to the mixture at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (86/14, v/v) to afford 1-(3-(2,4-dimethoxypyridin-3-yl)-1-((2-(trimethylsilyl)ethoxy)methyl)-1H-pyrrolo[2,3-b]pyridin-6-yl)-3-(3-(dimethylamino)propyl)urea (72.0 mg, 55%) as a light yellow solid. LCMS (ESI, m/z): [M+H]⁺=529.3.

### Step 5: Synthesis of 1-(3-(2,4-dimethoxypyridin-3-yl)-1H-pyrrolo[2,3-b]pyridin-6-yl)-3-(3-(dimethylamino)propyl)urea (Compound 195)

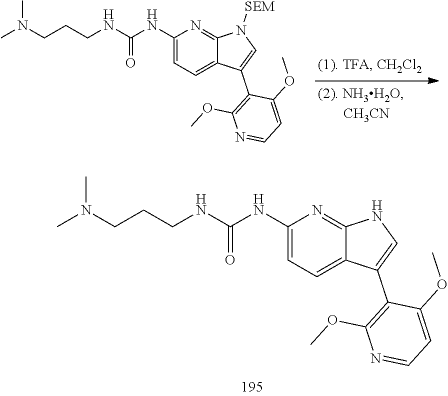 [SMILES: COc1ccnc(OC)c1-c1cn(COCC[Si](C)(C)C)c2nc(NC(=O)NCCCN(C)C)ccc12 ; COc1ccnc(OC)c1-c1c[nH]c2nc(NC(=O)NCCCN(C)C)ccc12]

To a solution of 1-(3-(2,4-dimethoxypyridin-3-yl)-1-((2-(trimethylsilyl)ethoxy)methyl)-1H-pyrrolo[2,3-b]pyridin-6-yl)-3-(3-(dimethylamino)propyl)urea (120.0 mg, 0.23 mmol) in CH₂Cl₂ (4.0 mL) was added TFA (4.0 mL) at room temperature. The resulting mixture was stirred at room temperature for 4 h. The resulting mixture was concentrated under reduced pressure. To the residue in CH₃CN (4.0 mL) was added NH₃·H₂O (4.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 17% B to 47% B in 9 min; 254 nm) to afford 1-(3-(2,4-dimethoxypyridin-3-yl)-1H-pyrrolo[2,3-b]pyridin-6-yl)-3-(3-(dimethylamino)propyl)urea (Compound 195) (14.6 mg, 16%) as a white solid. LCMS (ESI, m/z): [M+H]⁺=399.3. H NMR (400 MHz, DMSO-d₆): δ 11.45 (s, 1H), 9.13 (s, 1H), 8.57 (s, 1H), 8.05 (d, J=6.0 Hz, 1H), 7.53 (d, J=8.4 Hz, 1H), 7.26 (d, J=2.4 Hz, 1H), 6.95 (d, J=8.4 Hz, 1H), 6.87 (d, J=6.0 Hz, 1H), 3.80 (s, 3H), 3.79 (s, 3H), 3.33-3.21 (m, 1H), 3.25-3.23 (m, 1H), 2.34-2.23 (m, 2H), 2.17 (s, 6H), 1.68-1.62 (m, 2H).

### Example S186: Synthesis of (1S,2S)—N-[3-(7-chloro-5-methoxy-1,3-benzothiazol-6-yl)-1H-pyrrolo[2,3-b]pyridin-6-yl]-2-fluorocyclopropane-1-carboxamide (Compound 196)

### Step 1: Synthesis of 3-chloro-4-iodo-5-methoxyaniline

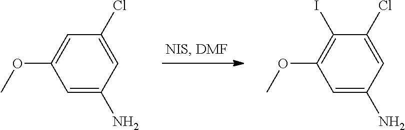

To a solution of 3-chloro-5-methoxyaniline (2.0 g, 12.69 mmol) in DMF (10.0 mL) was added NIS (2.6 g, 11.42 mmol) at 0° C. under N₂. The resulting mixture was stirred at room temperature for 0.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (85/15, v/v) to afford 3-chloro-4-iodo-5-methoxyaniline (1.6 g, 44%) as an off-white solid. LCMS (ESI, m/z): [M+H]⁺=283.9.

### Step 2: Synthesis of 1-benzoyl-3-(3-chloro-4-iodo-5-methoxyphenyl)thiourea

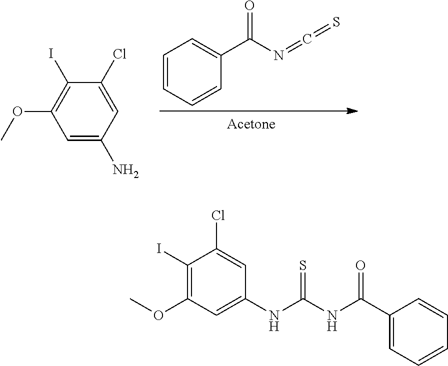

To a solution of 3-chloro-4-iodo-5-methoxyaniline (1.6 g, 5.64 mmol) in acetone (24.0 mL) was added benzoyl isothiocyanate (921.6 mg, 5.64 mmol) at 0° C. under N₂. The resulting mixture was stirred at 60° C. for 0.5 h. After the reaction was completed, the reaction mixture was filtered. The solid was collected and dried to afford 1-benzoyl-3-(3-chloro-4-iodo-5-methoxyphenyl)thiourea (2.0 g, crude) as a white solid. LCMS (ESI, m/z): [M+H]⁺=446.9.

### Step 3: Synthesis of 3-chloro-4-iodo-5-methoxyphenylthiourea

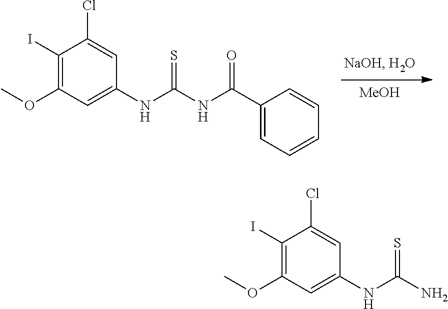

To a solution of 1-benzoyl-3-(3-chloro-4-iodo-5-methoxyphenyl)thiourea (2.0 g, 4.38 mmol) in MeOH/H₂O (10.0/10.0 mL) was added NaOH (193.3 mg, 4.83 mmol) at room temperature. The resulting mixture was stirred at room temperature for 16 h. After the reaction was completed, the mixture was filtered. The solid was collected and dried to afford 3-chloro-4-iodo-5-methoxyphenylthiourea (1.4 g, crude) as a white solid. LCMS (ESI, m/z): [M+H]⁺=342.9.

### Step 4: Synthesis of 7-chloro-6-iodo-5-methoxy-1,3-benzothiazol-2-amine

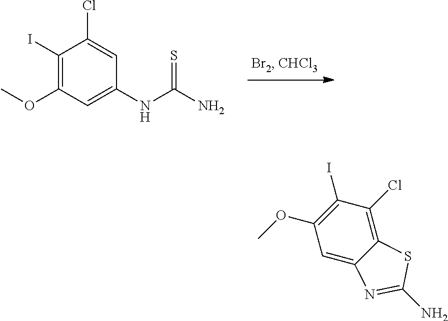

To a solution of 3-chloro-4-iodo-5-methoxyphenylthiourea (700.0 mg, 2.04 mmol) in CHCl₃ (8.0 mL) was added Br₂ (359.2 mg, 2.25 mmol) at room temperature. The resulting mixture was stirred at room temperature for 16 h. After the reaction was completed, the reaction was quenched with saturated NaHSO₃ solution at 0° C. The resulting mixture was extracted with ethyl acetate. The combined organic layer was washed with brine, dried over anhydrous sodium sulfate and filtered. The filtrate was concentrated under reduced pressure to afford 7-chloro-6-iodo-5-methoxy-1,3-benzothiazol-2-amine (560.0 mg, crude) as a white solid. LCMS (ESI, m/z): [M+H]⁺=340.9.

### Step 5: Synthesis of 7-chloro-6-iodo-5-methoxy-1,3-benzothiazole

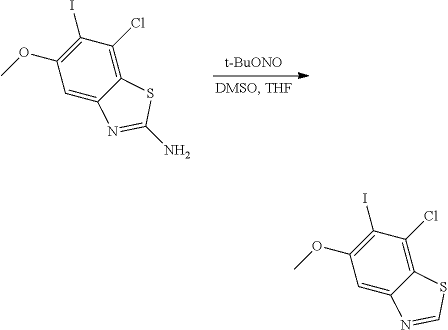

To a solution of 7-chloro-6-iodo-5-methoxy-1,3-benzothiazol-2-amine (560.0 mg, 1.72 mmol) in THF (10.0 mL) was added t-BuONO (265.3 mg, 2.57 mmol) and DMSO (10.7 mg, 0.14 mmol) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (85/15, v/v) to afford 7-chloro-6-iodo-5-methoxy-1,3-benzothiazole (250.0 mg, 44%) as a white solid. LCMS (ESI, m/z): [M+H]⁺=325.9.

### Step 6: Synthesis of 7-chloro-6-(6-chloro-1-{[2-(trimethylsilyl)ethoxy]methyl}pyrrolo[2,3-b]pyridin-3-yl)-5-methoxy-1,3-benzothiazole

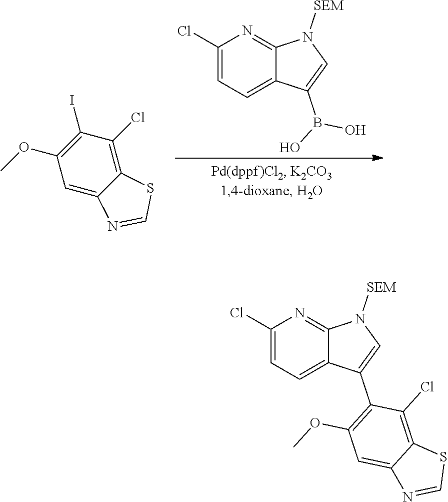 [SMILES: COc1cc2ncsc2c(Cl)c1I ; C[Si](C)(C)CCOCn1cc(B(O)O)c2ccc(Cl)nc21 ; COc1cc2ncsc2c(Cl)c1-c1cn(COCC[Si](C)(C)C)c2nc(Cl)ccc12]

To a solution of 7-chloro-6-iodo-5-methoxy-1,3-benzothiazole (220.0 mg, 0.68 mmol) in 1,4-dioxane/H₂O (8.0 mL/1.6 mL) was added 6-chloro-1-{[2-(trimethylsilyl)ethoxy]methyl}pyrrolo[2,3-b]pyridin-3-ylboronic acid (264.9 mg, 0.81 mmol), K₂CO₃ (280.2 mg, 2.03 mmol) and Pd(dppf)Cl₂ (55.0 mg, 0.07 mmol) at room temperature under N₂. The resulting mixture was stirred at 80° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford 7-chloro-6-(6-chloro-1-{[2-(trimethylsilyl)ethoxy]methyl}pyrrolo[2,3-b]pyridin-3-yl)-5-methoxy-1,3-benzothiazole (218.0 mg, 67%) as a yellow solid. LCMS (ESI, m/z): [M+H]⁺=480.1.

### Step 7: Synthesis of (1S,2S)—N-[3-(7-chloro-5-methoxy-1,3-benzothiazol-6-yl)-1-{[2-(trimethylsilyl)ethoxy]methyl}pyrrolo[2,3-b]pyridin-6-yl]-2-fluorocyclopropane-1-carboxamide

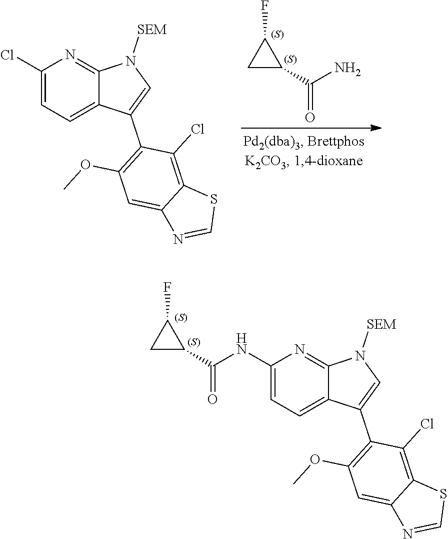

To a solution of 7-chloro-6-(6-chloro-1-{[2-(trimethylsilyl)ethoxy]methyl}pyrrolo[2,3-b]pyridin-3-yl)-5-methoxy-1,3-benzothiazole (188.0 mg, 0.39 mmol) in 1,4-dioxane (5.0 mL) was added (1S,2S)-2-fluorocyclopropane-1-carboxamide (201.7 mg, 1.96 mmol), K₂CO₃ (162.2 mg, 1.17 mmol), BrettPhos (42.0 mg, 0.08 mmol) and Pd₂(dba)₃ (35.8 mg, 0.04 mmol) at room temperature under N₂. The resulting mixture was stirred at room temperature for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum CH₂Cl₂/CH₃OH (95/5, v/v) to afford (1S,2S)—N-[3-(7-chloro-5-methoxy-1,3-benzothiazol-6-yl)-1-{[2-(trimethylsilyl)ethoxy]methyl}pyrrolo[2,3-b]pyridin-6-yl]-2-fluorocyclopropane-1-carboxamide (140.0 mg, 52%) as a yellow solid. LCMS (ESI, m/z): [M+H]⁺=547.1.

### Step 8: Synthesis of (1S,2S)—N-[3-(7-chloro-5-methoxy-1,3-benzothiazol-6-yl)-1H-pyrrolo[2,3-b]pyridin-6-yl]-2-fluorocyclopropane-1-carboxamide (Compound 196)

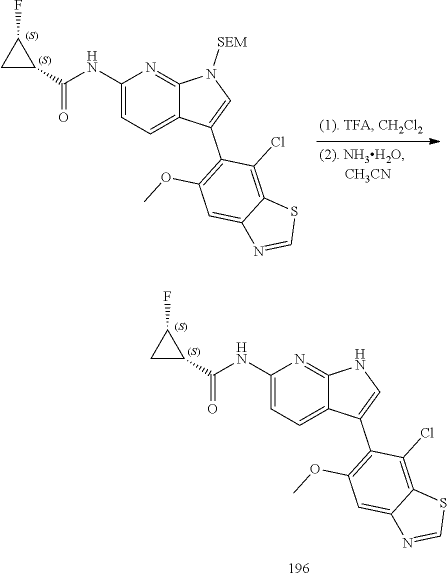

To a solution of (1S,2S)—N-[3-(7-chloro-5-methoxy-1,3-benzothiazol-6-yl)-1-{[2-(trimethylsilyl)ethoxy]methyl}pyrrolo[2,3-b]pyridin-6-yl]-2-fluorocyclopropane-1-carboxamide (130.0 mg, 0.24 mmol) in CH₂Cl₂ (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with CH₃CN/H₂O (70/30, v/v) to afford (1S,2S)—N-[3-(7-chloro-5-methoxy-1,3-benzothiazol-6-yl)-1H-pyrrolo[2,3-b]pyridin-6-yl]-2-fluorocyclopropane-1-carboxamide (Compound 196) (12.3 mg, 11%) as a white solid. LCMS (ESI, m/z): [M+H]⁺=417.1. ¹H NMR (400 MHz, DMSO-d₆): δ 11.77 (s, 1H), 10.68 (s, 1H), 9.45 (s, 1H), 7.87 (d, J=8.4 Hz, 1H), 7.79 (s 1H), 7.61 (d, J=8.8 Hz, 1H), 7.49 (d, J=2.0 Hz, 1H), 5.03-4.82 (m, 1H), 3.82 (s, 3H), 2.30-2.19 (m, 1H), 1.70-1.62 (m, 1H), 1.23-1.11 (m, 1H).

### Example S187: Synthesis of (1S,2S)-2-fluoro-N-[3-(5-fluoro-2-methoxy-4-methylpyridin-3-yl)-1H-pyrrolo[2,3-b]pyridin-6-yl]cyclopropane-1-carboxamide (Compound 197)

### Step 1: Synthesis of 5-fluoro-4-methylpyridin-2-ol

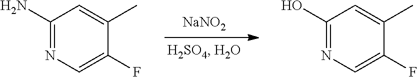

To a solution of 5-fluoro-4-methylpyridin-2-amine (600.0 mg, 4.76 mmol) in H₂SO₄ (12.0 mL)/H₂O (24.0 mL) was added dropwise a solution of NaNO₂ (1641.0 mg, 23.78 mmol) in H₂O (12.0 mL) at 0° C. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 5-fluoro-4-methylpyridin-2-ol (200.0 mg, crude) as a white solid. LCMS (ESI, m/z): [M+H]⁺=128.0.

### Step 2: Synthesis of 5-fluoro-3-iodo-4-methylpyridin-2-ol

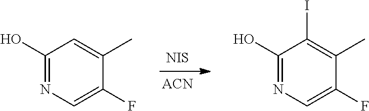

To a solution of 5-fluoro-4-methylpyridin-2-ol (820.0 mg, 6.45 mmol) in ACN (30.0 mL) was added NIS (1.6 g, 7.10 mmol) at room temperature. The resulting mixture was stirred at room temperature for 16 h. After the reaction was completed, the resulting mixture was concentrated under reduced pressure. The residue was purified by flash column chromatography with petroleum ether/ethyl acetate (1/4, v/v) to afford 5-fluoro-3-iodo-4-methylpyridin-2-ol (1.6 g, 98%) as a brown solid. LCMS (ESI, m/z): [M+H]⁺=253.9.

### Step 3: Synthesis of 5-fluoro-3-iodo-2-methoxy-4-methylpyridine

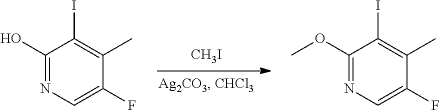

To a solution of 5-fluoro-3-iodo-4-methylpyridin-2-ol (900.0 mg, 3.56 mmol) in CHCl₃ (20.0 mL) was added Ag₂CO₃ (3923.4 mg, 14.23 mmol) and CH₃I (2019.6 mg, 14.23 mmol) at room temperature. The resulting mixture was stirred at 60° C. for 4 h. After the reaction was completed, the resulting mixture was filtered. The filtrate was concentrated under reduced pressure. The residue was purified by flash column chromatography with petroleum ether/ethyl acetate (10/1, v/v) to afford 5-fluoro-3-iodo-2-methoxy-4-methylpyridine (130.0 mg, 13%) as a white solid. LCMS (ESI, m/z): [M+H]⁺=268.0

### Step 4: Synthesis of 3-(6-chloro-1-[[2-(trimethylsilyl)ethoxy]methyl]pyrrolo[2,3-b]pyridin-3-yl)-5-fluoro-2-methoxy-4-methylpyridine

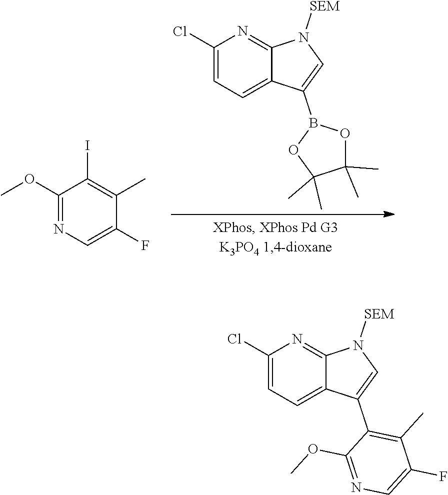

To a solution of 5-fluoro-3-iodo-2-methoxy-4-methylpyridine (380.0 mg, 1.42 mmol) in 1,4-dioxane/H₂O (10.0/2.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (581.7 mg, 1.42 mmol), XPhos (135.7 mg, 0.29 mmol), K₃PO₄ (906.2 mg, 4.27 mmol) and XPhos Pd G3 (120.5 mg, 0.14 mmol) at room temperature under N₂. The resulting mixture was stirred at 80° C. for 16 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 3-(6-chloro-1-[[2-(trimethylsilyl)ethoxy]methyl]pyrrolo[2,3-b]pyridin-3-yl)-5-fluoro-2-methoxy-4-methylpyridine (320.0 mg, 53%) as a colorless oil. LCMS (ESI, m/z): [M+H]⁺=422.1.

### Step 5: Synthesis of (1S,2S)-2-fluoro-N-[3-(5-fluoro-2-methoxy-4-methylpyridin-3-yl)-1-[[2-(trimethylsilyl)ethoxy]methyl]pyrrolo[2,3-b]pyridin-6-yl]cyclopropane-1-carboxamide

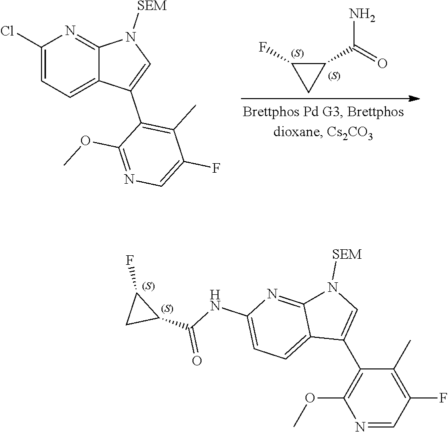

To a solution of 3-(6-chloro-1-[[2-(trimethylsilyl)ethoxy]methyl]pyrrolo[2,3-b]pyridin-3-yl)-5-fluoro-2-methoxy-4-methylpyridine (320.0 mg, 0.76 mmol) in 1,4-dioxane (10.0 mL) were added (1S,2S)-2-fluorocyclopropane-1-carboxamide (390.9 mg, 3.79 mmol), BrettPhos (81.4 mg, 0.15 mmol), Cs₂CO₃ (741.3 mg, 2.28 mmol) and BrettPhos Pd G3 (68.7 mg, 0.07 mmol) at room temperature under N₂. The resulting mixture was stirred at 100° C. for 4 h under N₂. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/3, v/v) to afford (1S,2S)-2-fluoro-N-[3-(5-fluoro-2-methoxy-4-methylpyridin-3-yl)-1-[[2-(trimethylsilyl)ethoxy]methyl]pyrrolo[2,3-b]pyridin-6-yl]cyclopropane-1-carboxamide (180.0 mg, 48%) as a colorless oil. LCMS (ESI, m/z): [M+H]⁺=489.2.

### Step 6: Synthesis of (1S,2S)-2-fluoro-N-[3-(5-fluoro-2-methoxy-4-methylpyridin-3-yl)-1H-pyrrolo[2,3-b]pyridin-6-yl]cyclopropane-1-carboxamide (Compound 197)

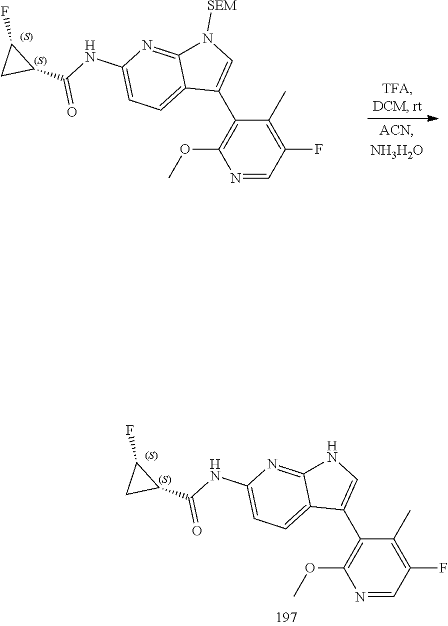

To a solution of (1S,2S)-2-fluoro-N-[3-(5-fluoro-2-methoxy-4-methylpyridin-3-yl)-1-[[2-(trimethylsilyl)ethoxy]methyl]pyrrolo[2,3-b]pyridin-6-yl]cyclopropane-1-carboxamide (180.0 mg, 0.37 mmol) in CH₂Cl₂ (3.0 mL) was added TFA (3.0 mL) at room temperature. The resulting mixture was stirred at room temperature for 4 h. The resulting mixture was concentrated under reduced pressure. To the residue in CH₃CN (3.0 mL) was added NH₃·H₂O (3.0 mL) at room temperature. The resulting mixture was stirred at room temperature for 3 h. After the reaction was completed, the resulting mixture was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 26% B to 56% B in 12 min; 254 nm) to afford (1S,2S)-2-fluoro-N-[3-(5-fluoro-2-methoxy-4-methylpyridin-3-yl)-1H-pyrrolo[2,3-b]pyridin-6-yl]cyclopropane-1-carboxamide (Compound 197) (27.0 mg, 20%) as a white solid. LCMS (ESI, m/z): [M+H]⁺=359.1. ¹H NMR (300 MHz, DMSO-d₆): δ 11.74 (s, 1H), 10.67 (s, 1H), 8.09 (s, 1H), 7.87 (d, J=8.1 Hz, 1H), 7.62 (d, J=8.4 Hz, 1H), 7.46 (s, 1H), 5.05-4.81 (m, 1H), 3.77 (s, 3H), 2.25-2.16 (m, 4H), 1.70-1.61 (m, 1H), 1.19-1.12 (m, 1H).

### Example S188: Synthesis of 3-[1-[2-(dimethylamino)ethyl]azetidin-3-yl]-1-[3-(2-methoxyphenyl)-1H-pyrrolo[2,3-b]pyridin-6-yl]urea (Compound 198)

### Step 1: Synthesis of tert-butyl 3-([[3-(2-methoxyphenyl)-1-[[2-(trimethylsilyl)ethoxy]methyl]pyrrolo[2,3-b]pyridin-6-yl]carbamoyl]amino)azetidine-1-carboxylate

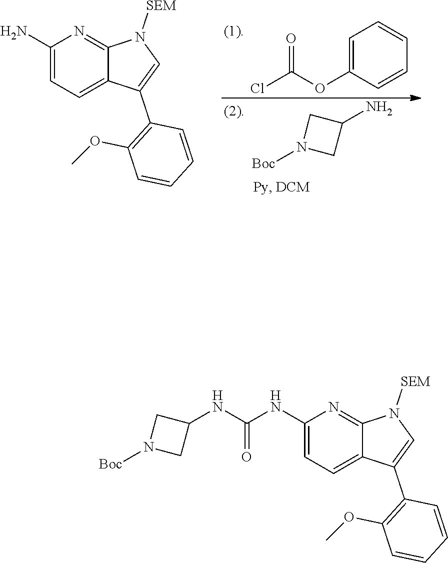

To a solution of 3-(2-methoxyphenyl)-1-[[2-(trimethylsilyl)ethoxy]methyl]pyrrolo[2,3-b]pyridin-6-amine (500.0 mg, 1.35 mmol) in CH₂Cl₂ (5.0 mL) was added pyridine (428.1 mg, 5.41 mmol) and phenyl chloroformate (254.2 mg, 1.62 mmol) at 0° C. The mixture was stirred at room temperature for 2 h. The mixture was concentrated under vacuum. To the above residue was added pyridine (5.0 mL) and tert-butyl 3-aminoazetidine-1-carboxylate (699.1 mg, 4.06 mmol). The resulting mixture was stirred at 60° C. for 2 h. After the reaction was completed, the resulting mixture was concentrated under vacuum. The residue was purified by flash column chromatography with CH₂Cl₂/MeOH (97/3, v/v) to afford tert-butyl 3-([[3-(2-methoxyphenyl)-1-[[2-(trimethylsilyl)ethoxy]methyl]pyrrolo[2,3-b]pyridin-6-yl]carbamoyl]amino)azetidine-1-carboxylate (600.0 mg, 78%) as a brown oil. LCMS (ESI, m/z): [M+H]⁺=568.3.

### Step 2: Synthesis of 3-(azetidin-3-yl)-1-[3-(2-methoxyphenyl)-1-[[2-(trimethylsilyl)ethoxy]methyl]pyrrolo[2,3-b]pyridin-6-yl]urea

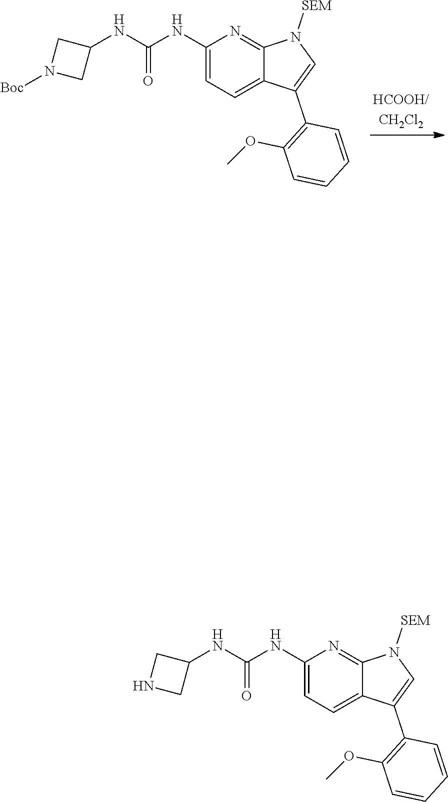 [SMILES: COc1ccccc1-c1cn(COCC[Si](C)(C)C)c2nc(NC(=O)NC3CN(C(=O)OC(C)(C)C)C3)ccc12 ; COc1ccccc1-c1cn(COCC[Si](C)(C)C)c2nc(NC(=O)NC3CNC3)ccc12]

To a solution of tert-butyl 3-([[3-(2-methoxyphenyl)-1-[[2-(trimethylsilyl)ethoxy]methyl]pyrrolo[2,3-b]pyridin-6-yl]carbamoyl]amino)azetidine-1-carboxylate (600.0 mg, 1.06 mmo) in CH₂Cl₂ (2.0 mL) was added HCOOH (4.0 mL). The resulting mixture was stirred at room temperature for 6 h. After the reaction was completed, The pH value of the mixture was adjusted to 7 with aq·NaHCO₃. The resulting mixture was extracted with CH₂Cl₂. The combined organic layer was washed with brine, dried over anhydrous sodium sulfate and filtered. The filtrate was concentrated under vacuum to afford 3-(azetidin-3-yl)-1-[3-(2-methoxyphenyl)-1-[[2-(trimethylsilyl)ethoxy]methyl]pyrrolo[2,3-b]pyridin-6-yl]urea (480.0 mg, crude) as a yellow solid. LCMS (ESI, m/z): [M+H]⁺=468.2.

### Step 3: Synthesis of 3-[1-[2-(dimethylamino)ethyl]azetidin-3-yl]-1-[3-(2-methoxyphenyl)-1-[[2-(trimethylsilyl)ethoxy]methyl]pyrrolo[2,3-b]pyridin-6-yl]urea

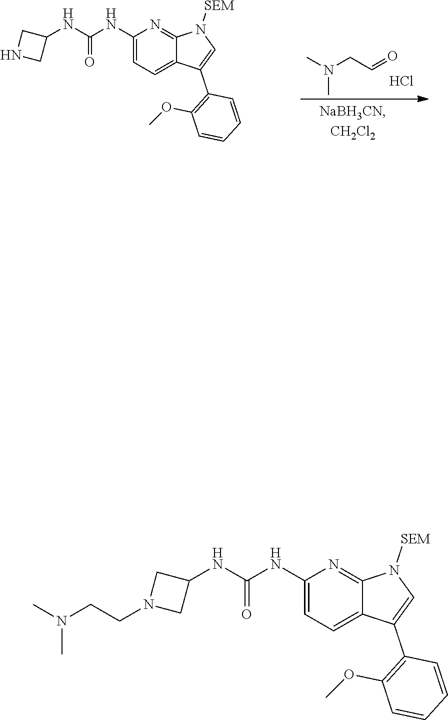

To a solution of 3-(azetidin-3-yl)-1-[3-(2-methoxyphenyl)-1-[[2-(trimethylsilyl)ethoxy]methyl]pyrrolo[2,3-b]pyridin-6-yl]urea (480.0 mg, crude) in CH₂Cl₂ (5.0 mL) was added 2-(dimethylamino)acetaldehyde hydrochloride (380.5 mg, 3.08 mmol) and NaBH₃CN (193.5 mg, 3.08 mmol) at room temperature. The mixture was stirred at room temperature for 2 h. After the reaction was completed, the resulting mixture was diluted with water and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (97/3, v/v) to afford 3-[1-[2-(dimethylamino)ethyl]azetidin-3-yl]-1-[3-(2-methoxyphenyl)-1-[[2-(trimethylsilyl)ethoxy]methyl]pyrrolo[2,3-b]pyridin-6-yl]urea (150.0 mg, 27%) as a yellow solid. LCMS (ESI, m/z): [M+H]⁺=539.3.

### Step 4: Synthesis of 3-[1-[2-(dimethylamino)ethyl]azetidin-3-yl]-1-[3-(2-methoxyphenyl)-1H-pyrrolo[2,3-b]pyridin-6-yl]urea (Compound 198)

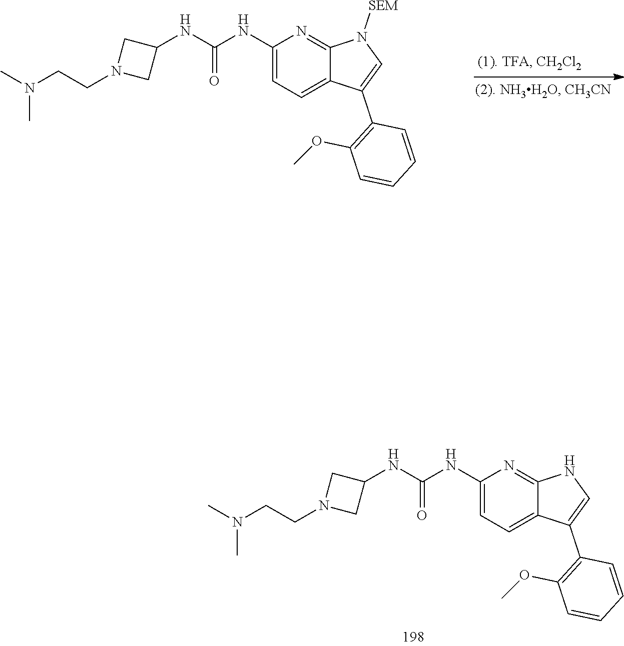 [SMILES: COc1ccccc1-c1cn(COCC[Si](C)(C)C)c2nc(NC(=O)NC3CN(CCN(C)C)C3)ccc12 ; COc1ccccc1-c1c[nH]c2nc(NC(=O)NC3CN(CCN(C)C)C3)ccc12]

To a solution of 3-[1-[2-(dimethylamino)ethyl]azetidin-3-yl]-1-[3-(2-methoxyphenyl)-1-[[2-(trimethylsilyl)ethoxy]methyl]pyrrolo[2,3-b]pyridin-6-yl]urea (150.0 mg, 0.28 mmol) in CH₂Cl₂ (3.0 mL) was added TFA (3.0 mL). The mixture was stirred at room temperature for 3 h. The mixture was concentrated under vacuum. To the above residue was added ACN (3.0 mL) and NH₃·H₂O (3.0 mL). The resulting mixture was stirred at room temperature for 3 h. After the reaction was completed, the resulting mixture was concentrated under vacuum. The residue was purified by Prep-HPLC with the following conditions (Column: XBridge Prep OBD Column, 30×150 mm, 5 um; Mobile Phase A: Water (10 mmol/L NH₄HCO₃), Mobile Phase B: ACN; Flow rate: 60 mL/min; Gradient: 26% B to 48% B in 8 min; 254 nm) to afford 3-[1-[2-(dimethylamino)ethyl]azetidin-3-yl]-1-[3-(2-methoxyphenyl)-1H-pyrrolo[2,3-b]pyridin-6-yl]urea (Compound 198) (11.4 mg, 10%) as a white solid. LCMS (ESI, m/z): [M+H]⁺=409.3. ¹H NMR (300 MHz, DMSO-d₆): δ 11.62 (s, 1H), 9.24 (s, 1H), 8.98-8.95 (m, 1H), 7.92 (d, J=8.4 Hz, 1H), 7.53-7.50 (m, 2H), 7.30-7.24 (m, 1H), 7.11 (d, J=7.5 Hz, 1H), 7.04-6.99 (m, 2H), 4.37-4.30 (m, 1H), 3.81 (s, 3H), 3.68-3.57 (m, 2H), 3.00-2.95 (m, 2H), 2.63-2.55 (m, 2H), 2.36-2.27 (m, 2H), 2.22 (s, 6H).

### Example S189: Synthesis of (1S,2S)-2-fluoro-N-(3-(5-methoxybenzo[d]thiazol-6-yl)-1H-pyrazolo[3,4-b]pyridin-6-yl)cyclopropane-1-carboxamide (Compound 199)

### Step 1: Synthesis of 6-(6-chloro-1-((2-(trimethylsilyl)ethoxy)methyl)-1H-pyrazolo[3,4-b]pyridin-3-yl)-5-methoxybenzo[d]thiazole

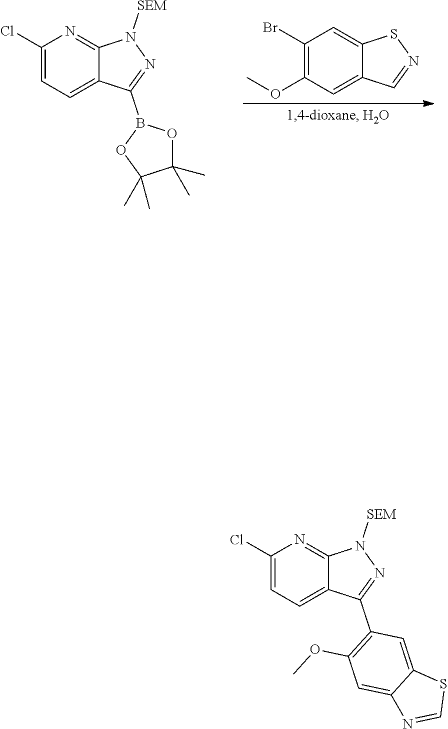 [SMILES: CC1(C)OB(c2nn(COCC[Si](C)(C)C)c3nc(Cl)ccc23)OC1(C)C ; COc1cc2cnsc2cc1Br ; COc1cc2ncsc2cc1-c1nn(COCC[Si](C)(C)C)c2nc(Cl)ccc12]

To a solution of 6-chloro-3-(4,4,5,5-tetramethyl-1,3,2-dioxaborolan-2-yl)-1-((2-(trimethylsilyl)ethoxy)methyl)-1H-pyrazolo[3,4-b]pyridine (350.0 mg, 1.07 mmol) in 1,4-dioxane (2.0 mL) was added 6-bromo-5-methoxy-1,3-benzothiazole (260.8 mg, 1.07 mmol), K₃PO₄ (680.2 mg, 3.21 mmol), Pd(AMPHOS)₂C₁₂ (90.8 mg, 0.13 mmol) and H₂O (0.5 mL) at room temperature under N₂. The resulting mixture was stirred at 80° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (60/40, v/v) to afford 6-(6-chloro-1-((2-(trimethylsilyl)ethoxy)methyl)-1H-pyrazolo[3,4-b]pyridin-3-yl)-5-methoxybenzo[d]thiazole (195.0 mg, 41%) as a white solid. LCMS (ESI, m/z): [M+H]⁺=447.1.

### Step 2: Synthesis of (1S,2S)-2-fluoro-N-(3-(5-methoxybenzo[d]thiazol-6-yl)-1-((2-(trimethylsilyl)ethoxy)methyl)-1H-pyrazolo[3,4-b]pyridin-6-yl)cyclopropane-1-carboxamide

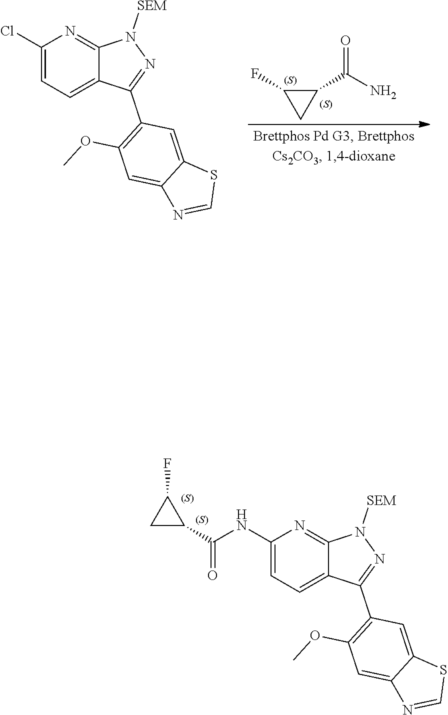

To a solution of 6-(6-chloro-1-((2-(trimethylsilyl)ethoxy)methyl)-1H-pyrazolo[3,4-b]pyridin-3-yl)-5-methoxybenzo[d]thiazole (150.0 mg, 0.34 mmol) in 1,4-dioxane (5.0 mL) was added (1S,2S)-2-fluorocyclopropane-1-carboxamide (173.0 mg, 1.68 mmol), K₂CO₃ (139.1 mg, 1.01 mmol) BrettPhos (36.0 mg, 0.07 mmol) and BrettPhos Pd G3 (30.4 mg, 0.03 mmol) at room temperature under N₂. The resulting mixture was stirred at 100° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (70/30, v/v) to afford (1S,2S)-2-fluoro-N-(3-(5-methoxybenzo[d]thiazol-6-yl)-1-((2-(trimethylsilyl)ethoxy)methyl)-1H-pyrazolo[3,4-b]pyridin-6-yl)cyclopropane-1-carboxamide (66.0 mg, 38%) as a yellow solid. LCMS (ESI, m/z): [M+H]⁺=514.2.

### Step 3: Synthesis of (1S,2S)-2-fluoro-N-(3-(5-methoxybenzo[d]thiazol-6-yl)-1H-pyrazolo[3,4-b]pyridin-6-yl)cyclopropane-1-carboxamide (Compound 199)

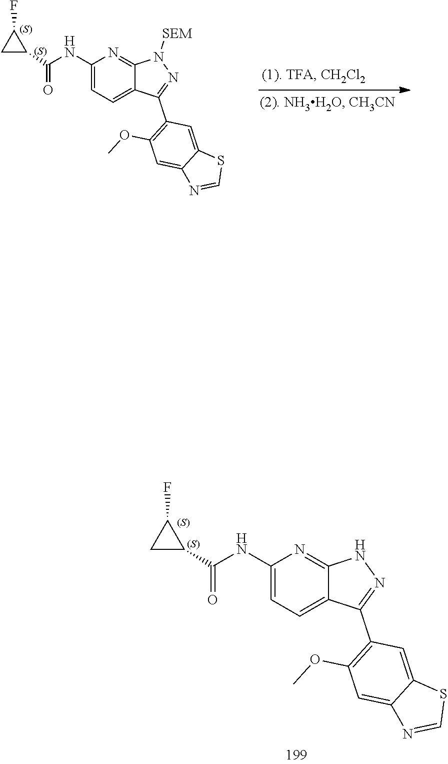

To a solution of (1S,2S)-2-fluoro-N-(3-(5-methoxybenzo[d]thiazol-6-yl)-1-((2-(trimethylsilyl)ethoxy)methyl)-1H-pyrazolo[3,4-b]pyridin-6-yl)cyclopropane-1-carboxamide (46.0 mg, 0.09 mmol) in DCM (1.0 mL) was added TFA (1.0 mL) at room temperature. The resulting mixture was stirred at room temperature for 4 h. The resulting mixture was concentrated under reduced pressure. To the residue in ACN (1.0 mL) was added NH₃·H₂O (1.0 mL) at room temperature. The resulting mixture was stirred at room temperature for another 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 ml/min; Gradient: 28% B to 35% B in 8 min; 254 nm) to afford (1S,2S)-2-fluoro-N-(3-(5-methoxybenzo[d]thiazol-6-yl)-1H-pyrazolo[3,4-b]pyridin-6-yl)cyclopropane-1-carboxamide (Compound 199) (15.3 mg, 44%) as a white solid. LCMS (ESI, m/z): [M+H]⁺=384.1. ¹H NMR (300 MHz, DMSO-d₆): δ 13.57 (s, 1H), 10.99 (s, 1H), 9.42 (s, 1H), 8.35 (s, 1H), 8.14 (d, J=8.8 Hz, 1H), 8.01 (d, J=9.2 Hz, 1H), 7.84 (s, 1H), 5.05-4.87 (m, 1H), 3.94 (s, 3H), 2.29-2.26 (m, 1H), 1.72-1.64 (m, 1H), 1.22-1.15 (m, 1H).

### Example S190: Synthesis of 1-(3-(2,6-dimethoxyphenyl)-1H-pyrrolo[2,3-b]pyridin-6-yl)-3-((1-methylazetidin-3-yl)methyl)urea (Compound 200)

### Step 1: Synthesis of Tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate

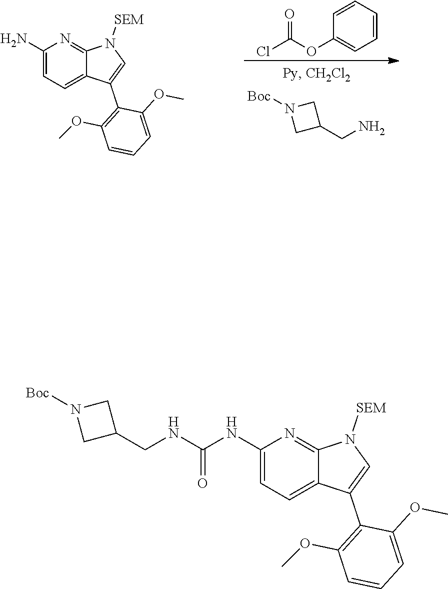 [SMILES: COc1cccc(OC)c1-c1cn(COCC[Si](C)(C)C)c2nc(N)ccc12 ; CC(C)(C)OC(=O)N1CC(CN)C1 ; O=C(Cl)Oc1ccccc1 ; COc1cccc(OC)c1-c1cn(COCC[Si](C)(C)C)c2nc(NC(=O)NCC3CN(C(=O)OC(C)(C)C)C3)ccc12]

To a solution of 3-(2,6-dimethoxyphenyl)-1-((2-(trimethylsilyl)ethoxy)methyl)-1H-pyrrolo[2,3-b]pyridin-6-amine (200.0 mg, 0.50 mmol) in CH₂Cl₂ (5.0 mL) was added pyridine (80.0 mg, 2.50 mmol) and phenyl chloroformate (156.7 mg, 1.0 mmol) at 0° C. under N₂. The resulting mixture was stirred at 0° C. for 1 h. The resulting mixture was concentrated under reduced pressure. To the above mixture in pyridine (5.0 mL) was added tert-butyl 3-(aminomethyl)azetidine-1-carboxylate (372.9 mg, 2.00 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (90/10, v/v) to afford tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate (150.0 mg, 49%) as a light yellow oil. LCMS (ESI, m/z): [M+H]⁺=612.3.

### Step 2: Synthesis of 1-(azetidin-3-ylmethyl)-3-(3-(2,6-dimethoxyphenyl)-1-((2-(trimethylsilyl)ethoxy)methyl)-1H-pyrrolo[2,3-b]pyridin-6-yl)urea

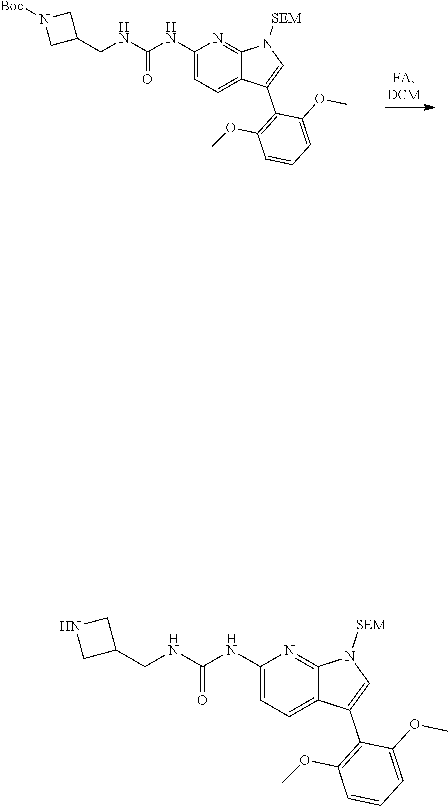 [SMILES: COc1cccc(OC)c1-c1cn(COCC[Si](C)(C)C)c2nc(NC(=O)NCC3CN(C(=O)OC(C)(C)C)C3)ccc12 ; COc1cccc(OC)c1-c1cn(COCC[Si](C)(C)C)c2nc(NC(=O)NCC3CNC3)ccc12]

To a solution of tert-butyl 3-((3-(3-(2,6-dimethoxyphenyl)-1-((2-(trimethylsilyl)ethoxy)methyl)-1H-pyrrolo[2,3-b]pyridin-6-yl)ureido)methyl)azetidine-1-carboxylate (100.0 mg, 0.16 mmol) in DCM (2.0 mL) was added FA (4.0 mL) at room temperature. The resulting mixture was stirred at room temperature for 2 h. After the reaction was completed, the pH value of the mixture was adjusted to 7 with aq·NaHCO₃ and then extracted with ethyl acetate. The combined organic layer was washed with brine, dried over anhydrous sodium sulfate and filtered. The filtrate was concentrated under reduced pressure to afford 1-(azetidin-3-ylmethyl)-3-(3-(2,6-dimethoxyphenyl)-1-((2-(trimethylsilyl)ethoxy)methyl)-1H-pyrrolo[2,3-b]pyridin-6-yl)urea (170.0 mg, crude) as a brown solid. LCMS (ESI, m/z): [M+H]⁺=512.3.

### Step 3: Synthesis of 1-(3-(2,6-dimethoxyphenyl)-1-((2-(trimethylsilyl)ethoxy)methyl)-1H-pyrrolo[2,3-b]pyridin-6-yl)-3-((1-methylazetidin-3-yl)methyl)urea

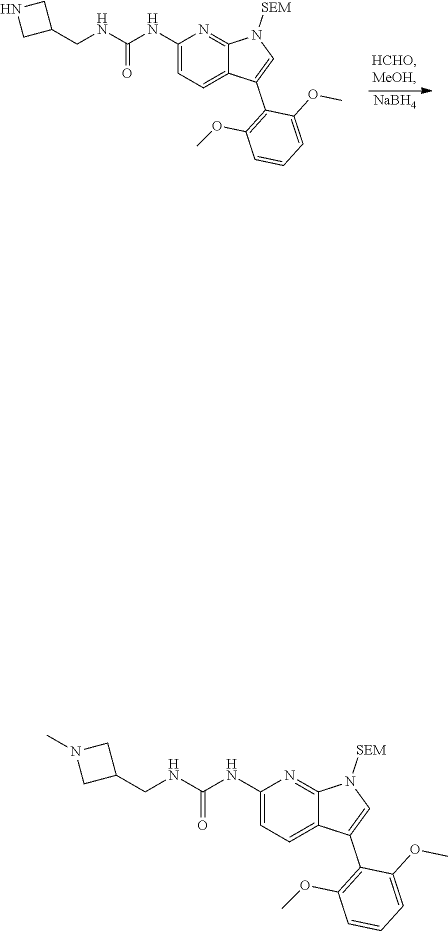

To a solution of 1-(azetidin-3-ylmethyl)-3-(3-(2,6-dimethoxyphenyl)-1-((2-(trimethylsilyl)ethoxy)methyl)-1H-pyrrolo[2,3-b]pyridin-6-yl)urea (170.0 mg, 0.33 mmol) in MeOH (3.0 mL) was added HCHO (50.0 mg, 0.67 mmol) and NaBH₄ (75.4 mg, 1.99 mmol) at room temperature under N₂. The resulting mixture was stirred at room temperature for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford 1-(3-(2,6-dimethoxyphenyl)-1-((2-(trimethylsilyl)ethoxy)methyl)-1H-pyrrolo[2,3-b]pyridin-6-yl)-3-((1-methylazetidin-3-yl)methyl)urea (190.0 mg, crude) as a brown solid. LCMS (ESI, m/z): [M+H]⁺=526.3.

### Step 4: Synthesis of 1-(3-(2,6-dimethoxyphenyl)-1H-pyrrolo[2,3-b]pyridin-6-yl)-3-((1-methylazetidin-3-yl)methyl)urea (Compound 200)

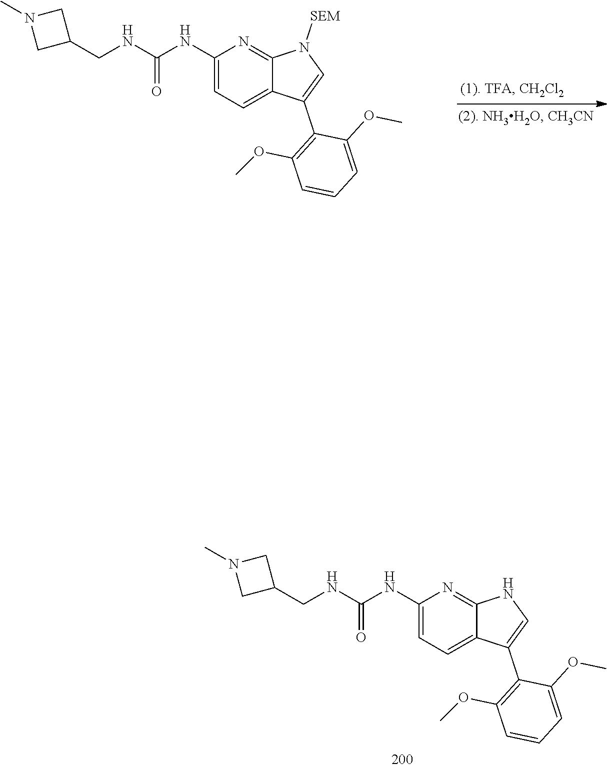

To a solution of 1-(3-(2,6-dimethoxyphenyl)-1-((2-(trimethylsilyl)ethoxy)methyl)-1H-pyrrolo[2,3-b]pyridin-6-yl)-3-((1-methylazetidin-3-yl)methyl)urea (200.0 mg, 0.38 mmol) in CH₂Cl₂ (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% B to 40% B in 8 min; 254 nm) to afford 1-(3-(2,6-dimethoxyphenyl)-1H-pyrrolo[2,3-b]pyridin-6-yl)-3-((1-methylazetidin-3-yl)methyl)urea (Compound 200) (5.2 mg, 3%) as a white solid. LCMS (ESI, m/z): [M+H]⁺=396.2. ¹H NMR (400 MHz, DMSO-d₆): δ 11.37 (s, 1H), 9.14 (s, 1H), 8.82 (s, 1H), 7.46 (d, J=8.4 Hz, 1H), 7.28-7.24 (m, 1H), 7.16 (d, J=2.4 Hz, 1H), 6.88 (d, J=8.8 Hz, 1H), 6.74 (d, J=8.8 Hz, 2H), 3.68 (s, 6H), 3.40-3.37 (m, 3H), 3.30-3.24 (m, 2H), 2.92-2.85 (m, 2H), 2.18 (s, 3H).

### Example S191: Synthesis of (1S,2S)—N-[3-(2-ethoxy-5-fluoro-4-methoxypyridin-3-yl)-1H-pyrrolo[2,3-b]pyridin-6-yl]-2-fluorocyclopropane-1-carboxamide (Compound 201)

### Step 1: Synthesis of 3-bromo-2-chloro-5-fluoropyridin-4-ol

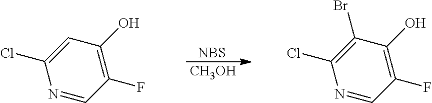

To a solution of 2-chloro-5-fluoropyridin-4-ol (2.0 g, 13.55 mmol) in CH₃OH (10.0 mL) was added NBS (2.9 g, 16.26 mmol) at 0° C. under N₂. The resulting mixture was stirred at 0° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (92/8, v/v) to afford 3-bromo-2-chloro-5-fluoropyridin-4-ol (3.0 g, 97%) as a yellow solid. LCMS (ESI, m/z): [M+H]⁺=225.9.

### Step 2: Synthesis of 3-bromo-2-chloro-5-fluoro-4-methoxypyridine

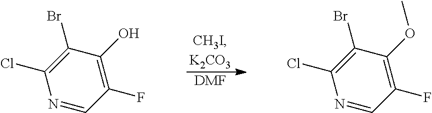

To a solution of 3-bromo-2-chloro-5-fluoropyridin-4-ol (1.5 g, 6.62 mmol) in DMF (5.0 mL) was added K₂CO₃ (1.8 g, 13.25 mmol) and CH₃I (1.1 g, 7.95 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (60/40, v/v) to afford 3-bromo-2-chloro-5-fluoro-4-methoxypyridine (600.0 mg, 37%) as a white solid. LCMS (ESI, m/z): [M+H]⁺=239.9.

### Step 3: Synthesis of 3-bromo-2-ethoxy-5-fluoro-4-methoxypyridine

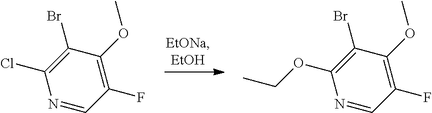

To a solution of 3-bromo-2-chloro-5-fluoro-4-methoxypyridine (600.0 mg, 2.50 mmol) in EtOH (5.0 mL) was added EtONa (203.6 mg, 3.00 mmol) at room temperature under N₂. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (65/35, v/v) to afford 3-bromo-2-ethoxy-5-fluoro-4-methoxypyridine (500.0 mg, 80%) as a white solid. LCMS (ESI, m/z): [M+H]⁺=250.0.

### Step 4: Synthesis of 3-(6-chloro-1-[[2-(trimethylsilyl)ethoxy]methyl]pyrrolo[2,3-b]pyridin-3-yl)-2-ethoxy-5-fluoro-4-methoxypyridine

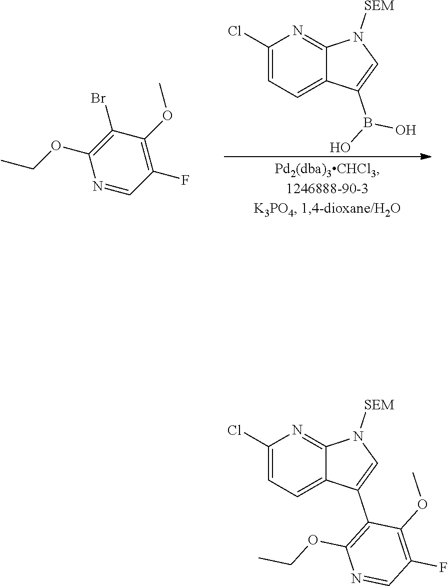 [SMILES: CCOc1ncc(F)c(OC)c1Br ; C[Si](C)(C)CCOCn1cc(B(O)O)c2ccc(Cl)nc21 ; CCOc1ncc(F)c(OC)c1-c1cn(COCC[Si](C)(C)C)c2nc(Cl)ccc12]

To a solution of 3-bromo-2-ethoxy-5-fluoro-4-methoxypyridine (400.0 mg, 1.60 mmol) in 1,4-dioxane/H₂O (5.0/1.0 mL) was added 6-chloro-1-[[2-(trimethylsilyl)ethoxy]methyl]pyrrolo[2,3-b]pyridin-3-ylboronic acid (627.0 mg, 1.92 mmol), K₃PO₄ (679.2 mg, 3.20 mmol), 3-tert-butyl-4-(2,6-dimethoxyphenyl)-2,3-dihydro[d]-[1,3]-oxaphosphole (CAS 1246888-90-3) (52.8 mg, 0.16 mmol) and Pd₂(dba)₃ CHCl₃ (80.0 mg, 0.08 mmol) at room temperature under N₂. The resulting mixture was stirred at 90° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (0/100, v/v) to afford 3-(6-chloro-1-[[2-(trimethylsilyl)ethoxy]methyl]pyrrolo[2,3-b]pyridin-3-yl)-2-ethoxy-5-fluoro-4-methoxypyridine (300.0 mg, 55%) as a white solid. LCMS (ESI, m/z): [M+H]⁺=452.1.

### Step 5: Synthesis of (1S,2S)—N-[3-(2-ethoxy-5-fluoro-4-methoxypyridin-3-yl)-1-[[2-(trimethylsilyl)ethoxy]methyl]pyrrolo[2,3-b]pyridin-6-yl]-2-fluorocyclopropane-1-carboxamide

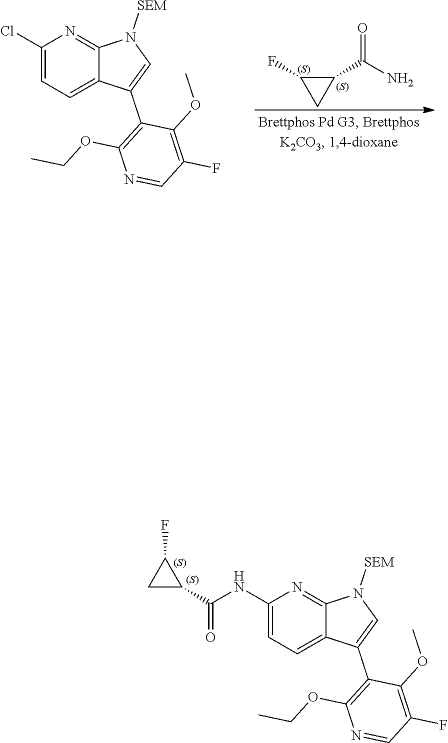

To a solution of 3-(6-chloro-1-[[2-(trimethylsilyl)ethoxy]methyl]pyrrolo[2,3-b]pyridin-3-yl)-2-ethoxy-5-fluoro-4-methoxypyridine (300.0 mg, 0.66 mmol) in 1,4-dioxane (5.0 mL) was added (1S,2S)-2-fluorocyclopropane-1-carboxamide (342.1 mg, 3.32 mmol), Cs₂CO₃ (648.7 mg, 1.99 mmol), BrettPhos (71.1 mg, 0.13 mmol) and BrettPhos Pd G3 (60.6 mg, 0.07 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (0/100, v/v) to afford (1S,2S)—N-[3-(2-ethoxy-5-fluoro-4-methoxypyridin-3-yl)-1-[[2-(trimethylsilyl)ethoxy]methyl]pyrrolo[2,3-b]pyridin-6-yl]-2-fluorocyclopropane-1-carboxamide (160.0 mg, 46%) as a yellow solid. LCMS (ESI, m/z): [M+H]⁺=519.2.

### Step 6: Synthesis of (1S,2S)—N-[3-(2-ethoxy-5-fluoro-4-methoxypyridin-3-yl)-1H-pyrrolo[2,3-b]pyridin-6-yl]-2-fluorocyclopropane-1-carboxamide (Compound 201)

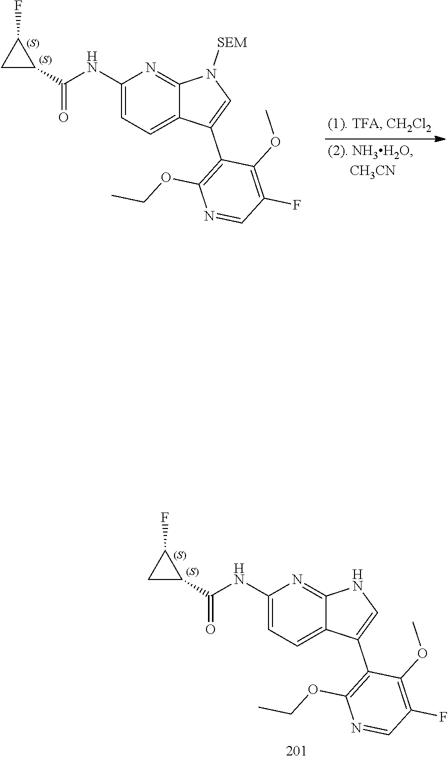

To a solution of (1S,2S)—N-[3-(2-ethoxy-5-fluoro-4-methoxypyridin-3-yl)-1-[[2-(trimethylsilyl)ethoxy]methyl]pyrrolo[2,3-b]pyridin-6-yl]-2-fluorocyclopropane-1-carboxamide (160.0 mg, 0.31 mmol) in CH₂Cl₂ (3.0 mL) was added TFA (3.0 mL) at room temperature. The resulting mixture was stirred at room temperature for 3 h. The resulting mixture was concentrated under reduced pressure. To the residue in CH₃CN (3.0 mL) was added NH₃·H₂O (3.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 17% B to 27% B in 9 min, 254 nm) to afford (1S,2S)—N-[3-(2-ethoxy-5-fluoro-4-methoxypyridin-3-yl)-1H-pyrrolo[2,3-b]pyridin-6-yl]-2-fluorocyclopropane-1-carboxamide (Compound 201) (37.5 mg, 31%) as a white solid. LCMS (ESI, m/z): [M+H]⁺=389.1. ¹H NMR (400 MHz, DMSO-d₆): δ 11.58 (s, 1H), 10.63 (s, 1H), 8.06 (d, J=7.2 Hz, 1H), 7.84 (d, J=8.8 Hz, 1H), 7.75 (d, J=8.4 Hz, 1H), 7.59 (d, J=2.4 Hz, 1H), 5.00-4.83 (m, 1H), 3.63-3.57 (m, 5H), 2.27-2.18 (m, 1H), 1.67-1.61 (m, 1H), 1.17-1.12 (m, 1H), 0.96-0.90 (m, 3H).

### Example S192: Synthesis of 1-[3-(2,6-dimethoxyphenyl)-1H-pyrrolo[2,3-b]pyridin-6-yl]-3-([1-[(dimethylamino)methyl]cyclopropyl]methyl)urea (Compound 202)

### Step 1: Synthesis of 1-[3-(2,6-dimethoxyphenyl)-1-[[2-(trimethylsilyl)ethoxy]methyl]pyrrolo[2,3-b]pyridin-6-yl]-3-([1-[(dimethylamino)methyl]cyclopropyl]methyl)urea

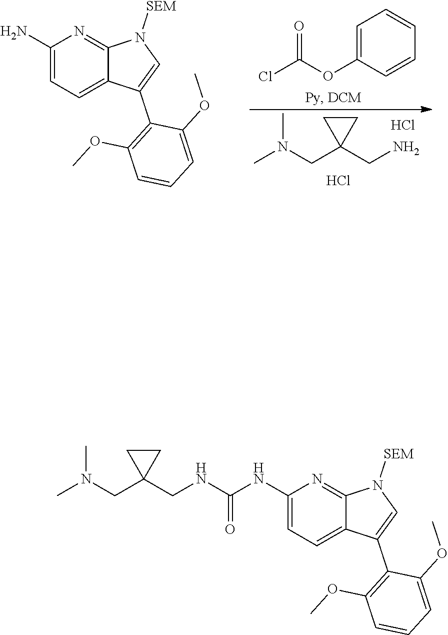

To a solution of 3-(2,6-dimethoxyphenyl)-1-[[2-(trimethylsilyl)ethoxy]methyl]pyrrolo[2,3-b]pyridin-6-amine (180.0 mg, 0.45 mmol) in DCM (10.0 mL) were added pyridine (142.5 mg, 1.80 mmol) and phenyl chloroformate (141.1 mg, 0.90 mmol) at room temperature. The resulting mixture was stirred at room temperature for 3 h. The resulting mixture was concentrated under reduced pressure. To the above mixture were added 1-[1-[(dimethylamino)methyl]cyclopropyl]methanamine dihydrochloride (453.1 mg, 2.25 mmol) and pyridine (10.0 mL) at room temperature. The resulting mixture was stirred at 60° C. for 16 h. After the reaction was completed, the resulting mixture was concentrated under reduced pressure. The residue was purified by flash column chromatography with CH₂Cl₂/methanol (6/1, v/v) to afford 1-[3-(2,6-dimethoxyphenyl)-1-[[2-(trimethylsilyl)ethoxy]methyl]pyrrolo[2,3-b]pyridin-6-yl]-3-([1-[(dimethylamino)methyl]cyclopropyl]methyl)urea (174.0 mg, 69%) as a light brown solid. LCMS (ESI, m/z): [M+H]⁺=554.3.

### Step 2: Synthesis of 1-[3-(2,6-dimethoxyphenyl)-1H-pyrrolo[2,3-b]pyridin-6-yl]-3-([1-[(dimethylamino)methyl]cyclopropyl]methyl)urea (Compound 202)

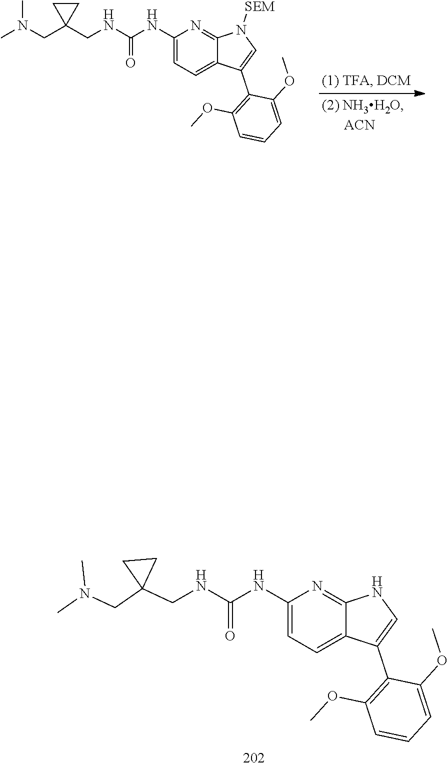 [SMILES: COc1cccc(OC)c1-c1cn(COCC[Si](C)(C)C)c2nc(NC(=O)NCC3(CN(C)C)CC3)ccc12 ; COc1cccc(OC)c1-c1c[nH]c2nc(NC(=O)NCC3(CN(C)C)CC3)ccc12]

To a solution of 1-[3-(2,6-dimethoxyphenyl)-1-[[2-(trimethylsilyl)ethoxy]methyl]pyrrolo[2,3-b]pyridin-6-yl]-3-([1-[(dimethylamino)methyl]cyclopropyl]methyl)urea (174.0 mg, 0.31 mmol) in CH₂Cl₂ (8.0 mL) was added TFA (8.0 mL) at room temperature. The resulting mixture was stirred at room temperature for 3 h. The resulting mixture was concentrated under reduced pressure. To the residue in CH₃CN (8.0 mL) was added NH₃·H₂O (8.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 47% B in 10 min; 254/220 nm) to afford 1-[3-(2,6-dimethoxyphenyl)-1H-pyrrolo[2,3-b]pyridin-6-yl]-3-([1-[(dimethylamino)methyl]cyclopropyl]methyl)urea (Compound 202) (30.6 mg, 22%) as a white solid. LCMS (ESI, m/z): [M+H]⁺=424.2. ¹H NMR (400 MHz, DMSO-d₆): δ 11.26 (d, J=1.6 Hz, 1H), 9.11 (s, 1H), 8.46 (s, 1H), 7.46 (d, J=8.8 Hz, 1H), 7.28-7.24 (m, 1H), 7.15 (d, J=2.4 Hz, 1H), 6.96 (d, J=8.4 Hz, 1H), 6.74 (d, J=8.4 Hz, 2H), 3.68 (s, 6H), 3.33-3.24 (m, 2H), 2.20-2.18 (m, 8H), 0.54-0.51 (m, 2H), 0.29-0.26 (m, 2H).

### Example S193: Synthesis of 1-(3-(4,6-dimethoxy-2-methylpyrimidin-5-yl)-1H-pyrrolo[2,3-b]pyridin-6-yl)-3-(2-(4-ethylpiperazin-1-yl)ethyl)urea (Compound 203)

### Step 1: Synthesis of 3-(4,6-dimethoxy-2-methylpyrimidin-5-yl)-1-((2-(trimethylsilyl)ethoxy)methyl)-1H-pyrrolo[2,3-b]pyridin-6-amine

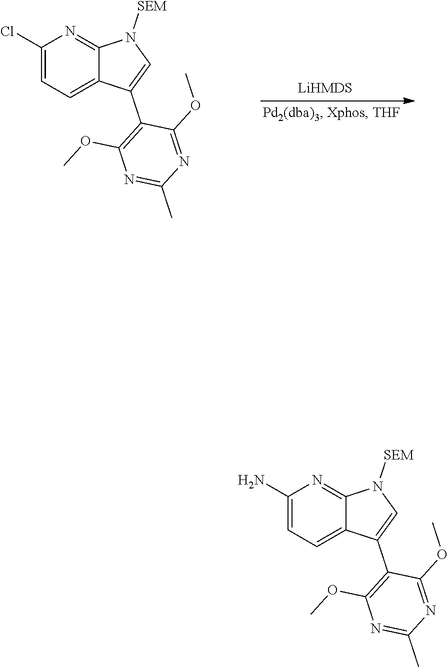 [SMILES: COc1nc(C)nc(OC)c1-c1cn(COCC[Si](C)(C)C)c2nc(Cl)ccc12 ; COc1nc(C)nc(OC)c1-c1cn(COCC[Si](C)(C)C)c2nc(N)ccc12]

To a solution of 6-chloro-3-(4,6-dimethoxy-2-methylpyrimidin-5-yl)-1-((2-(trimethylsilyl)ethoxy)methyl)-1H-pyrrolo[2,3-b]pyridine (200.0 mg, 0.46 mmol) in THE (10.0 mL) was added LiHMDS (0.9 mL, 1 mol/L), Xphos (43.8 mg, 0.09 mmol) and Pd₂(dba)₃ (42.1 mg, 0.05 mmol) at room temperature under N₂. The resulting mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 3-(4,6-dimethoxy-2-methylpyrimidin-5-yl)-1-((2-(trimethylsilyl)ethoxy)methyl)-1H-pyrrolo[2,3-b]pyridin-6-amine (160.0 mg, 83%) as a yellow oil. LCMS (ESI, m/z): [M+H]⁺=416.2.

### Step 2: Synthesis of 1-(3-(4,6-dimethoxy-2-methylpyrimidin-5-yl)-1-((2-(trimethylsilyl)ethoxy)methyl)-1H-pyrrolo[2,3-b]pyridin-6-yl)-3-(2-(4-ethylpiperazin-1-yl)ethyl)urea

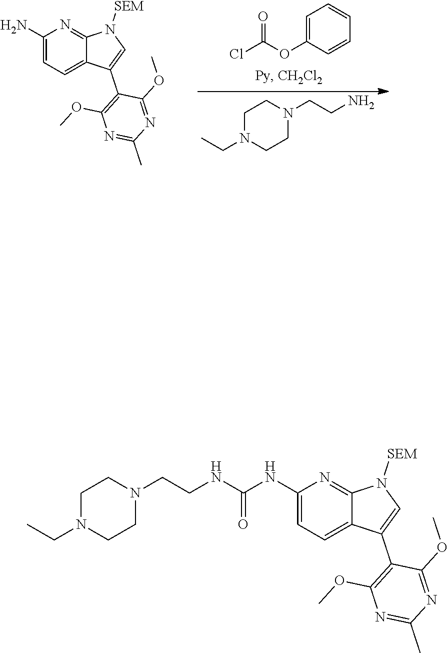 [SMILES: COc1nc(C)nc(OC)c1-c1cn(COCC[Si](C)(C)C)c2nc(N)ccc12 ; CCN1CCN(CCN)CC1 ; O=C(Cl)Oc1ccccc1 ; CCN1CCN(CCNC(=O)Nc2ccc3c(-c4c(OC)nc(C)nc4OC)cn(COCC[Si](C)(C)C)c3n2)CC1]

To a solution of 3-(4,6-dimethoxy-2-methylpyrimidin-5-yl)-1-((2-(trimethylsilyl)ethoxy)methyl)-1H-pyrrolo[2,3-b]pyridin-6-amine (130.0 mg, 0.31 mmol) in CH₂Cl₂ (5.0 mL) was added phenyl carbonochloridate (146.9 mg, 0.94 mmol) and pyridine (100.2 mg, 1.25 mmol) at room temperature. The resulting mixture was stirred at room temperature for 3 h. The resulting mixture was concentrated under reduced pressure. To the residue in pyridine (5.0 mL) was added 2-(4-ethylpiperazin-1-yl)ethan-1-amine (59.0 mg, 0.38 mmol) at room temperature. The resulting mixture was stirred at 60° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 1-(3-(4,6-dimethoxy-2-methylpyrimidin-5-yl)-1-((2-(trimethylsilyl)ethoxy)methyl)-1H-pyrrolo[2,3-b]pyridin-6-yl)-3-(2-(4-ethylpiperazin-1-yl)ethyl)urea (80.0 mg, 42%) as a yellow solid. LCMS (ESI, m/z): [M+H]⁺=599.3.

### Step 3: Synthesis of 1-(3-(4,6-dimethoxy-2-methylpyrimidin-5-yl)-1H-pyrrolo[2,3-b]pyridin-6-yl)-3-(2-(4-ethylpiperazin-1-yl)ethyl)urea (Compound 203)

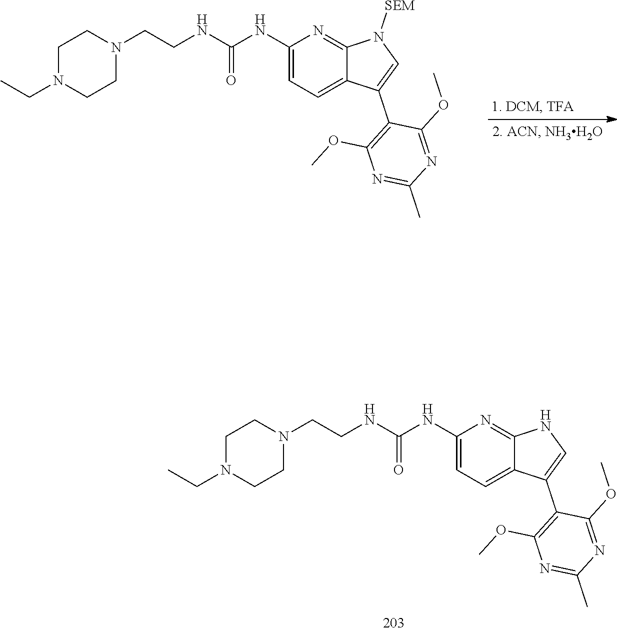 [SMILES: CCN1CCN(CCNC(=O)Nc2ccc3c(-c4c(OC)nc(C)nc4OC)cn(COCC[Si](C)(C)C)c3n2)CC1 ; CCN1CCN(CCNC(=O)Nc2ccc3c(-c4c(OC)nc(C)nc4OC)c[nH]c3n2)CC1]

To a solution of 1-(3-(4,6-dimethoxy-2-methylpyrimidin-5-yl)-1-((2-(trimethylsilyl)ethoxy)methyl)-1H-pyrrolo[2,3-b]pyridin-6-yl)-3-(2-(4-ethylpiperazin-1-yl)ethyl)urea (70.0 mg, 0.12 mmol) in DCM (2.0 mL) was added TFA (2.0 mL) at room temperature. The resulting mixture was stirred at room temperature for 3 h. The resulting mixture was concentrated under reduced pressure. To the residue in ACN (2.0 mL) was added NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 31% to 41% in 8 min; 254 nm) to afford 1-(3-(4,6-dimethoxy-2-methylpyrimidin-5-yl)-1H-pyrrolo[2,3-b]pyridin-6-yl)-3-(2-(4-ethylpiperazin-1-yl)ethyl)urea (Compound 203) (12.1 mg, 22%) as a yellow solid. LCMS (ESI, m/z): [M+H]⁺=469.4. ¹H NMR (300 MHz, CDCl₃): δ 10.14 (s, 1H), 9.73 (s, 1H), 7.64 (d, J=8.4 Hz, 1H), 7.36 (d, J=2.4 Hz, 1H), 6.43 (d, J=8.4 Hz, 1H), 3.99 (s, 6H), 3.66-3.60 (m, 2H), 2.90-2.83 (m, 6H), 2.66-2.52 (m, 9H), 1.21-1.16 (m, 3H).

### Example S194: Synthesis of 1-[3-(4-cyclopropoxy-2-methoxypyridin-3-yl)-1H-pyrrolo[2,3-b]pyridin-6-yl]-3-[(2R)-3-(dimethylamino)-2-fluoropropyl]urea and 1-[3-(4-cyclopropoxy-2-methoxypyridin-3-yl)-1H-pyrrolo[2,3-b]pyridin-6-yl]-3-[(2S)-3-(dimethylamino)-2-fluoropropyl]urea (Compound 204 and Compound 205)

### Step 1: Synthesis of 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)-2-fluoropropyl]urea

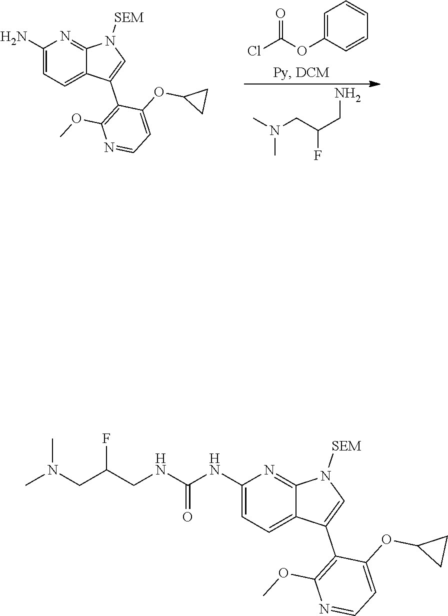

To a solution of 3-(4-cyclopropoxy-2-methoxypyridin-3-yl)-1-{[2-(trimethylsilyl)ethoxy]methyl}pyrrolo[2,3-b]pyridin-6-amine (375.0 mg, 0.88 mmol) in DCM (15.0 mL) was added pyridine (278.1 mg, 3.52 mmol) and phenyl chloroformate (275.3 mg, 1.76 mmol) at room temperature. The resulting mixture was stirred at room temperature for 3 h. The resulting mixture was concentrated under reduced pressure. To the above residue was added (3-amino-2-fluoropropyl)dimethylamine (950.7 mg, 7.91 mmol) and pyridine (15.0 mL) at room temperature. The resulting mixture was stirred at 70° C. for 16 h. After the reaction was completed, the resulting mixture was concentrated under reduced pressure. The residue was purified by flash column chromatography with CH₂Cl₂/CH₃OH (11/1, v/v) to afford 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)-2-fluoropropyl]urea (420.0 mg, 83%) as a light yellow oil. LCMS (ESI, m/z): [M+H]⁺=573.3.

### Step 2: Synthesis of 1-[3-(4-cyclopropoxy-2-methoxypyridin-3-yl)-1H-pyrrolo[2,3-b]pyridin-6-yl]-3-[3-(dimethylamino)-2-fluoropropyl]urea

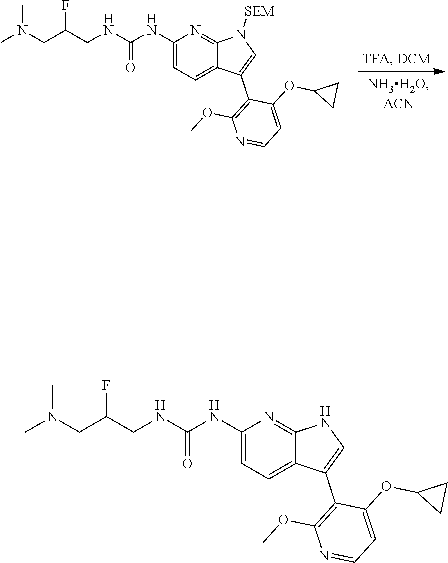 [SMILES: COc1nccc(OC2CC2)c1-c1cn(COCC[Si](C)(C)C)c2nc(NC(=O)NCC(F)CN(C)C)ccc12 ; COc1nccc(OC2CC2)c1-c1c[nH]c2nc(NC(=O)NCC(F)CN(C)C)ccc12]

To a solution of 1-[3-(4-cyclopropoxy-2-methoxypyridin-3-yl)-1-{[2-(trimethylsilyl)ethoxy]methyl}pyrrolo[2,3-b]pyridin-6-yl]-3-[3-(dimethylamino)-2-fluoropropyl]urea (270.0 mg, 0.47 mmol) in CH₂Cl₂ (10.0 mL) was added TFA (10.0 mL) at room temperature. The resulting mixture was stirred at room temperature for 3 h. The resulting mixture was concentrated under reduced pressure. To the residue in CH₃CN (10.0 mL) was added NH₃·H₂O (10.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with acetonitrile/water (1/1, v/v) to afford 1-[3-(4-cyclopropoxy-2-methoxypyridin-3-yl)-1H-pyrrolo[2,3-b]pyridin-6-yl]-3-[3-(dimethylamino)-2-fluoropropyl]urea (95.0 mg, 45%) as a light yellow solid. LCMS (ESI, m/z): [M+H]⁺=443.2.

### Step 3: Synthesis of 1-[3-(4-cyclopropoxy-2-methoxypyridin-3-yl)-1H-pyrrolo[2,3-b]pyridin-6-yl]-3-[(2R)-3-(dimethylamino)-2-fluoropropyl]urea and 1-[3-(4-cyclopropoxy-2-methoxypyridin-3-yl)-1H-pyrrolo[2,3-b]pyridin-6-yl]-3-[(2S)-3-(dimethylamino)-2-fluoropropyl]urea (Compound 204 and Compound 205)

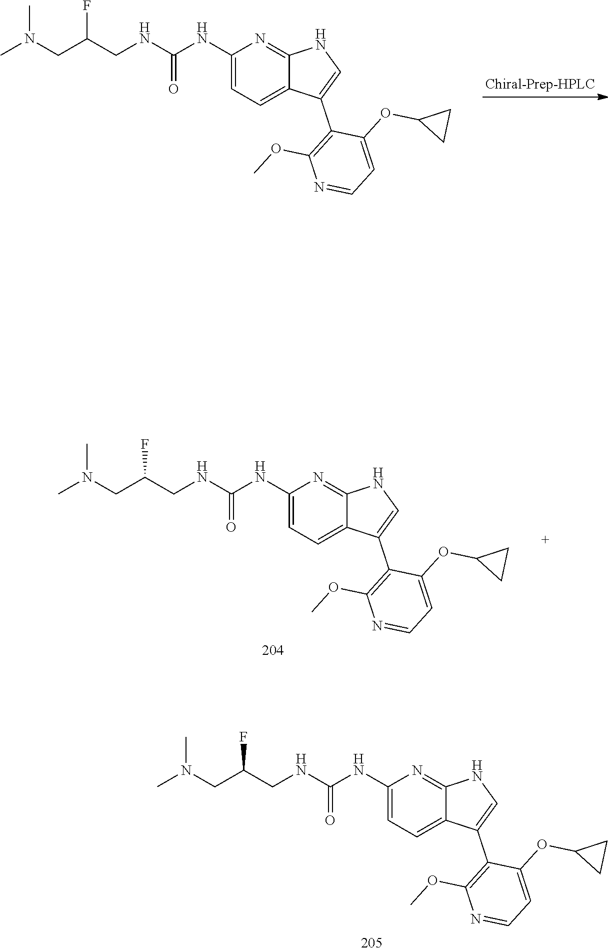 [SMILES: COc1nccc(OC2CC2)c1-c1c[nH]c2nc(NC(=O)NCC(F)CN(C)C)ccc12 ; COc1nccc(OC2CC2)c1-c1c[nH]c2nc(NC(=O)NC[C@@H](F)CN(C)C)ccc12 ; COc1nccc(OC2CC2)c1-c1c[nH]c2nc(NC(=O)NC[C@H](F)CN(C)C)ccc12]

The racemic 1-[3-(4-cyclopropoxy-2-methoxypyridin-3-yl)-1H-pyrrolo[2,3-b]pyridin-6-yl]-3-[3-(dimethylamino)-2-fluoropropyl]urea (95.0 mg, 0.22 mmol) was separated by Prep-Chiral—HPLC with the following conditions (Column: CHIRAL ART Amylose-SA, 2×25 cm, 5 m; Mobile Phase A: Hex (0.5% 2M NH₃-MeOH)—HPLC, Mobile Phase B: EtOH—HPLC; Flow rate: 20 mL/min; Gradient: 50% B to 50% B in 21 min; Wave Length: 220/254 nm) to afford 1-[3-(4-cyclopropoxy-2-methoxypyridin-3-yl)-1H-pyrrolo[2,3-b]pyridin-6-yl]-3-[3-(dimethylamino)-2-fluoropropyl]urea Enantiomer 1 (31.4 mg, 66%) as a white solid and 1-[3-(4-cyclopropoxy-2-methoxypyridin-3-yl)-1H-pyrrolo[2,3-b]pyridin-6-yl]-3-[3-(dimethylamino)-2-fluoropropyl]urea Enantiomer 2 (35.4 mg, 74%) as a white solid. The absolute stereochemistry of Enantiomers 1 and 2 was not assigned. The two enantiomeric structures that could be obtained from chiral separation of the enantiomeric mixture as described above are shown as Compounds 204 and 205 in Table 1.

1-[3-(4-cyclopropoxy-2-methoxypyridin-3-yl)-1H-pyrrolo[2,3-b]pyridin-6-yl]-3-[3-(dimethylamino)-2-fluoropropyl]urea Enantiomer 1: Retention Time 1=6.66 min; LCMS (ESI, m/z): [M+H]⁺=443.1. ¹H NMR (400 MHz, DMSO-d₆): δ 11.49 (s, 1H), 9.23 (s, 1H), 8.62 (s, 1H), 8.07 (d, J=5.6 Hz, 1H), 7.51 (d, J=8.4 Hz, 1H), 7.23 (d, J=2.0 Hz, 1H), 7.13 (d, J=6.0 Hz, 1H), 7.00 (d, J=8.8 Hz, 1H), 4.81-4.68 (m, 1H), 3.94-3.91 (m, 1H), 3.81 (s, 3H), 3.65-3.52 (m, 1H), 3.45-3.35 (m, 1H), 2.56-2.51 (m, 2H), 2.22 (s, 6H), 0.81-0.76 (m, 2H), 0.63-0.58 (m, 2H).

1-[3-(4-cyclopropoxy-2-methoxypyridin-3-yl)-1H-pyrrolo[2,3-b]pyridin-6-yl]-3-[3-(dimethylamino)-2-fluoropropyl]urea Enantiomer 2: Retention Time 2=14.70 min; LCMS (ESI, m/z): [M+H]⁺=443.1. ¹H NMR (400 MHz, DMSO-d₆): δ 11.49 (s, 1H), 9.23 (s, 1H), 8.63 (s, 1H), 8.06 (d, J=5.6 Hz, 1H), 7.51 (d, J=8.8 Hz, 1H), 7.23 (d, J=2.0 Hz, 1H), 7.13 (d, J=6.0 Hz, 1H), 7.00 (d, J=8.4 Hz, 1H), 4.82-4.69 (m, 1H), 3.93-3.90 (m, 1H), 3.81 (s, 3H), 3.63-3.52 (m, 1H), 3.45-3.34 (m, 1H), 2.57-2.51 (m, 2H), 2.23 (s, 6H), 0.81-0.76 (m, 2H), 0.65-0.63 (m, 2H).

### Example S195: Synthesis of 1-(3-{5,7-dimethoxy-[1,3]thiazolo[4,5-b]pyridin-6-yl}-1H-pyrrolo[2,3-b]pyridin-6-yl)-3-[(2R)-3-(dimethylamino)-2-fluoropropyl]urea and 1-(3-{5, 7-dimethoxy-[1,3]thiazolo[4,5-b]pyridin-6-yl}-1H-pyrrolo[2,3-b]pyridin-6-yl)-3-[(2S)-3-(dimethylamino)-2-fluoropropyl]urea (Compound 206 and Compound 207)

### Step 1: Synthesis of 1-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-3-[3-(dimethylamino)-2-fluoropropyl]urea

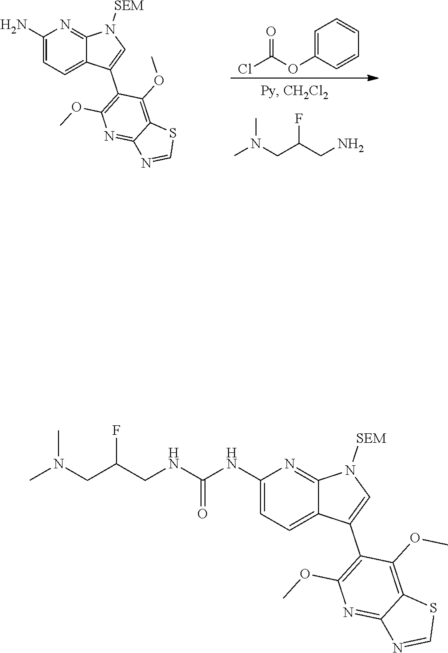

To a solution of 3-{5,7-dimethoxy-[1,3]thiazolo[4,5-b]pyridin-6-yl}-1-{[2-(trimethylsilyl)ethoxy]methyl}pyrrolo[2,3-b]pyridin-6-amine (300.0 mg, 0.66 mmol) in CH₂Cl₂ (4.0 mL) was added pyridine (207.4 mg, 2.62 mmol) and phenyl chloroformate (123.2 mg, 0.79 mmol) at room temperature. The resulting mixture was stirred at room temperature for 1 h. The resulting mixture was concentrated under reduced pressure. To the residue in pyridine (6.0 mL) was added (3-amino-2-fluoropropyl)dimethylamine (787.8 mg, 6.56 mmol) at room temperature. The resulting mixture was stirred at 60° C. for 1 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum CH₂Cl₂/CH₃OH (90/10, v/v) to afford 1-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-3-[3-(dimethylamino)-2-fluoropropyl]urea (250.0 mg, 63%) as a yellow solid. LCMS (ESI, m/z): [M+H]⁺=604.2.

### Step 2: Synthesis of 1-(3-{5,7-dimethoxy-[1,3]thiazolo[4,5-b]pyridin-6-yl}-1H-pyrrolo[2,3-b]pyridin-6-yl)-3-[(2R)-3-(dimethylamino)-2-fluoropropyl]urea and 1-(3-{5,7-dimethoxy-[1,3]thiazolo[4,5-b]pyridin-6-yl}-1H-pyrrolo[2,3-b]pyridin-6-yl)-3-[(2S)-3-(dimethylamino)-2-fluoropropyl]urea (Compound 206 and Compound 207)

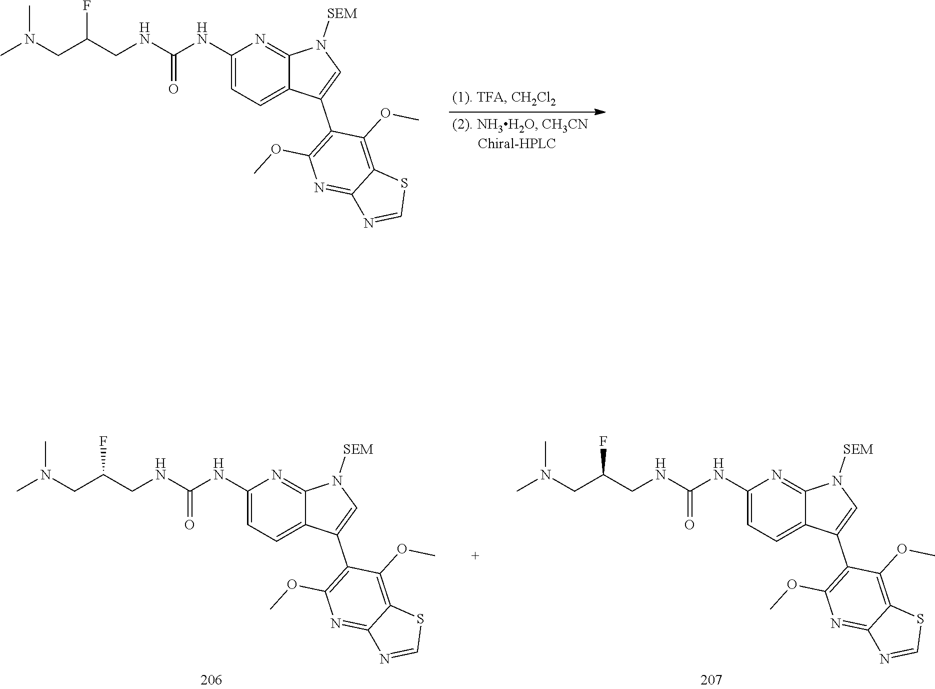

To a solution of 1-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-3-[3-(dimethylamino)-2-fluoropropyl]urea (250.0 mg, 0.41 mmol) in CH₂Cl₂ (2.0 mL) was added TFA (2.0 mL) at room temperature. The resulting mixture was stirred at room temperature for 4 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (52/48, v/v) and then separated by Prep-Chiral-HPLC with the following conditions (Column: CHIRAL ART Amylose-SA, 2×25 cm, 5 m; Mobile Phase A: Hex (0.5% 2M NH₃-MeOH)—HPLC, Mobile Phase B: EtOH—HPLC; Flow rate: 20 mL/min; Gradient: 50% B to 50% B in 10.5 min; Wave Length: 220/254 nm) to afford 1-(3-{5,7-dimethoxy-[1,3]thiazolo[4,5-b]pyridin-6-yl}-1H-pyrrolo[2,3-b]pyridin-6-yl)-3-[3-(dimethylamino)-2-fluoropropyl]urea Enantiomer 1 (30.9 mg, 31%) as a white solid and 1-(3-{5,7-dimethoxy-[1,3]thiazolo[4,5-b]pyridin-6-yl}-1H-pyrrolo[2,3-b]pyridin-6-yl)-3-[3-(dimethylamino)-2-fluoropropyl]urea Enantiomer 2 (40.4 mg, 41%) as a white solid. The absolute stereochemistry of Enantiomers 1 and 2 was not assigned. The two enantiomeric structures that could be obtained from chiral separation of the enantiomeric mixture as described above are shown as Compounds 206 and 207 in Table 1.

1-(3-{5,7-dimethoxy-[1,3]thiazolo[4,5-b]pyridin-6-yl}-1H-pyrrolo[2,3-b]pyridin-6-yl)-3-[3-(dimethylamino)-2-fluoropropyl]urea Enantiomer 1: Retention Time 1=5.876 min; LCMS (ESI, m/z): [M+H]⁺=474.2. ¹H NMR (400 MHz, DMSO-d₆): δ 11.62 (s, 1H), 9.56 (s, 1H), 9.27 (s, 1H), 8.62 (s, 1H), 7.65 (d, J=8.8 Hz, 1H), 7.37 (d, J=2.0 Hz, 1H), 7.05 (d, J=8.8 Hz, 1H), 4.82-4.69 (m, 1H), 3.91-3.86 (m, 6H), 3.65-3.53 (m, 1H), 3.46-3.33 (m, 1H), 2.56-2.51 (m, 2H), 2.21 (s, 6H).

1-(3-{5,7-dimethoxy-[1,3]thiazolo[4,5-b]pyridin-6-yl}-1H-pyrrolo[2,3-b]pyridin-6-yl)-3-[3-(dimethylamino)-2-fluoropropyl]urea Enantiomer 2: Retention Time 2=9.106 min; LCMS (ESI, m/z): [M+H]⁺=474.2. ¹H NMR (400 MHz, DMSO-d₆): δ 11.62 (s, 1H), 9.56 (s, 1H), 9.27 (s, 1H), 8.62 (s, 1H), 7.65 (d, J=8.4 Hz, 1H), 7.37 (s, 1H), 7.05 (d, J=8.8 Hz, 1H), 4.82-4.69 (m, 1H), 3.91 and 3.89 (s, total 6H), 3.62-3.54 (m, 1H), 3.44-3.33 (m, 1H), 2.67-2.46 (m, 2H), 2.21 (s, 6H).

### Example S196: Synthesis of (1S,2S)—N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide and (1R,2R)—N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide (Compound 208 and Compound 209)

### Step 1: Synthesis of Methyl (trans)-2-((3-(4-cyclopropoxy-6-methoxypyrimidin-5-yl)-1-((2-(trimethylsilyl)ethoxy)methyl)-1H-pyrrolo[2,3-b]pyridin-6-yl)carbamoyl)cyclopropane-1-carboxylate

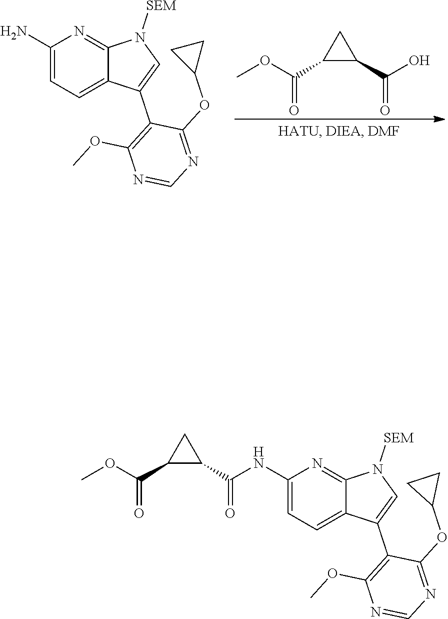

To a solution of 3-(4-cyclopropoxy-6-methoxypyrimidin-5-yl)-1-{[2-(trimethylsilyl)ethoxy]methyl}pyrrolo[2,3-b]pyridin-6-amine (1.0 g, 2.34 mmol) in DMF (20.0 mL) was added trans-2-(methoxycarbonyl)cyclopropane-1-carboxylic acid (404.5 mg, 2.81 mmol), DIEA (1.5 g, 11.70 mmol) and HATU (1.3 g, 3.51 mmol) at 0 C under N₂. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (86/14, v/v) to afford methyl (trans)-2-{[3-(4-cyclopropoxy-6-methoxypyrimidin-5-yl)-1-{[2-(trimethylsilyl)ethoxy]methyl}pyrrolo[2,3-b]pyridin-6-yl]carbamoyl}cyclopropane-1-carboxylate (510.0 mg, 39%) as a yellow oil. LCMS (ESI, m/z): [M+H]⁺=554.2.

### Step 2: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide

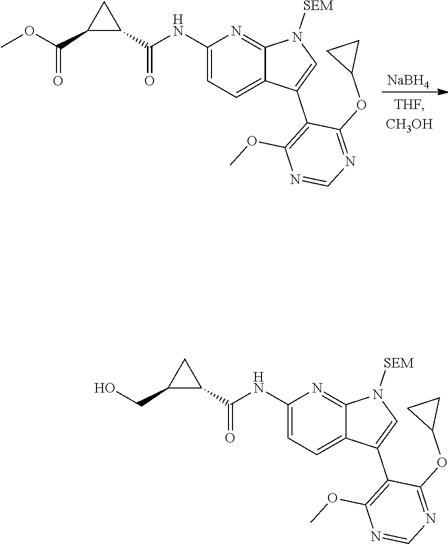

To a solution of methyl (trans)-2-{[3-(4-cyclopropoxy-6-methoxypyrimidin-5-yl)-1-{[2-(trimethylsilyl)ethoxy]methyl}pyrrolo[2,3-b]pyridin-6-yl]carbamoyl}cyclopropane-1-carboxylate (460.0 mg, 0.83 mmol) in THF/CH₃OH (12.0/3.0 mL) was added NaBH₄ (3.1 g, 83.10 mmol) at room temperature. The resulting mixture was stirred at 30° C. for 16 h. After the reaction was completed, the reaction mixture was quenched with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (57/43, v/v) to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide (265.0 mg, 61%) as a white solid. LCMS (ESI, m/z): [M+H]⁺=526.2.

### Step 3: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide

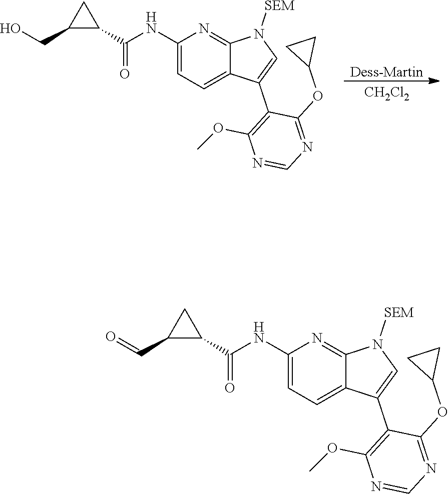

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide (250.0 mg, 0.48 mmol) in CH₂Cl₂ (10.0 mL) was added Dess-Martin (302.6 mg, 0.71 mmol) at 0° C. under N₂. The resulting mixture was stirred at room temperature for 2 h. After the reaction was completed, the resulting mixture was diluted with water and extracted with CH₂Cl₂. The combined organic layer was washed with brine, dried over anhydrous sodium sulfate and filtered. The filtrate was concentrated under reduced pressure to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide (250.0 mg, crude) as a yellow solid. LCMS (ESI, m/z): [M+H]⁺=524.2.

### Step 4: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(dimethylamino)methyl]cyclopropane-1-carboxamide

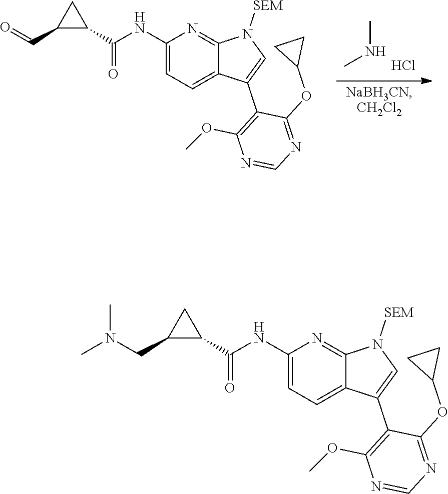

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide (250.0 mg, crude) in CH₂Cl₂ (10.0 mL) were added dimethylamine hydrochloride (116.8 mg, 1.43 mmol) and NaBH₃CN (90.0 mg, 1.43 mmol) at 0° C. under N₂. The resulting mixture was stirred at room temperature for 2 h. After the reaction was completed, the resulting mixture was diluted with water and extracted with CH₂Cl₂. The combined organic layer was washed with brine, dried over anhydrous sodium sulfate and filtered. The filtrate was concentrated under reduced pressure. The residue was purified by flash column chromatography with petroleum CH₂Cl₂/CH₃OH (10/01, v/v) to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(dimethylamino)methyl]cyclopropane-1-carboxamide (150.0 mg, 57%) as a yellow oil. LCMS (ESI, m/z): [M+H]⁺=553.3.

### Step 5: Synthesis of Trans-N-(3-(4-cyclopropoxy-6-methoxypyrimidin-5-yl)-1H-pyrrolo[2,3-b]pyridin-6-yl)-2-((dimethylamino)methyl)cyclopropane-1-carboxamide

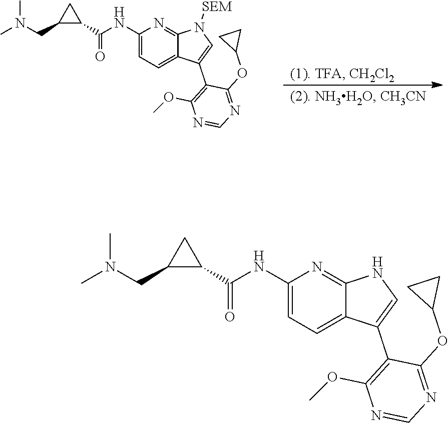 [SMILES: COc1ncnc(OC2CC2)c1-c1cn(COCC[Si](C)(C)C)c2nc(NC(=O)[C@H]3C[C@@H]3CN(C)C)ccc12 ; "(1). TFA, CH2Cl2" ; "(2). NH3·H2O, CH3CN" ; COc1ncnc(OC2CC2)c1-c1c[nH]c2nc(NC(=O)[C@H]3C[C@@H]3CN(C)C)ccc12]

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(dimethylamino)methyl]cyclopropane-1-carboxamide (130.0 mg, 0.24 mmol) in CH₂Cl₂ (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 20% B to 30% B in 8 min, 30% B; Wave Length: 254 nm) to afford trans-N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide (50.0 mg, 50%) as a white solid. LCMS (ESI, m/z): [M+H]⁺=423.2.

### Step 6: Synthesis of (1S,2S)—N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide and (1R,2R)—N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide (Compound 208 and Compound 209)

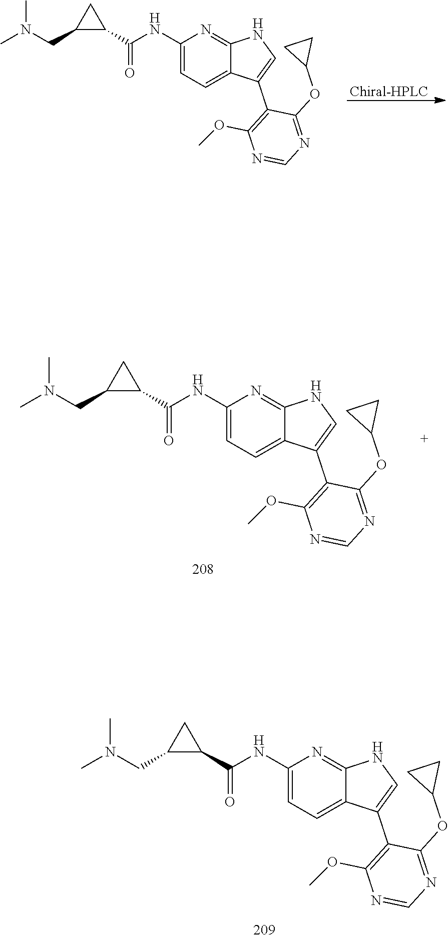 [SMILES: COc1ncnc(OC2CC2)c1-c1c[nH]c2nc(NC(=O)[C@H]3C[C@@H]3CN(C)C)ccc12 ; "Chiral-HPLC" ; COc1ncnc(OC2CC2)c1-c1c[nH]c2nc(NC(=O)[C@H]3C[C@@H]3CN(C)C)ccc12 ; COc1ncnc(OC2CC2)c1-c1c[nH]c2nc(NC(=O)[C@@H]3C[C@H]3CN(C)C)ccc12]

The product trans-N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide (50.0 mg, 0.12 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)—HPLC, Mobile Phase B: MeOH: EtOH=1: 1—HPLC; Flow rate: 20 mL/min; Gradient: 30% B to 30% B in 12.5 min; Wave Length: 220/254 nm) to afford N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide Enantiomer 1 (6.6 mg, 26%) and N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide Enantiomer 2 (5.4 mg, 22%) as a white solid. The absolute stereochemistry of Enantiomers 1 and 2 was not assigned. The two enantiomeric structures that could be obtained from chiral separation of the enantiomeric mixture as described above are shown as Compounds 208 and 209 in Table 1.

N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide Enantiomer 1: Retention Time 1=9.02 min; LCMS (ESI, m/z): [M+H]⁺=423.4. ¹H NMR (400 MHz, DMSO-d₆): δ 11.62 (s, 1H), 10.54 (s, 1H), 8.49 (s, 1H), 7.85 (d, J=8.8 Hz, 1H), 7.65 (d, J=8.8 Hz, 1H), 7.44 (d, J=2.4 Hz, 1H), 4.38-4.35 (m, 1H), 3.92 (s, 3H), 2.31-2.27 (m, 1H), 2.19-2.14 (m, 7H), 1.90-1.87 (m, 1H), 1.35-1.33 (m, 1H), 1.07-1.03 (m, 1H), 0.76-0.70 (m, 3H), 0.69-0.62 (m, 2H).

N-[3-(4-cyclopropoxy-6-methoxypyrimidin-5-yl)-1H-pyrrolo[2,3-b]pyridin-6-yl]-2-[(dimethylamino)methyl]cyclopropane-1-carboxamide Enantiomer 2: Retention Time 2=11.39 min; LCMS (ESI, m/z): [M+H]⁺=423.4. ¹H NMR (400 MHz, DMSO-d₆): δ 11.62 (s, 1H), 10.54 (s, 1H), 8.49 (s, 1H), 7.86 (d, J=8.8 Hz, 1H), 7.65 (d, J=8.8 Hz, 1H), 7.44 (d, J=2.4 Hz, 1H), 4.38-4.35 (m, 1H), 3.92 (s, 3H), 2.31-2.26 (m, 1H), 2.19-2.13 (m, 7H), 1.91-1.88 (m, 1H), 1.35-1.33 (m, 1H), 1.07-1.03 (m, 1H), 0.78-0.62 (m, 5H). $$

### Example S197: Synthesis of 1-(3-(4-cyclopropoxy-2-methoxypyridin-3-yl)-1H-pyrrolo[2,3-b]pyridin-6-yl)-3-(2-(4-methylpiperazin-1-yl)ethyl)urea formate (Compound 210)

### Step 1: Synthesis of 3-[3-(4-cyclopropoxy-2-methoxypyridin-3-yl)-1-{[2-(trimethylsilyl)ethoxy]methyl}pyrrolo[2,3-b]pyridin-6-yl]-1-[2-(4-methylpiperazin-1-yl)ethyl]urea

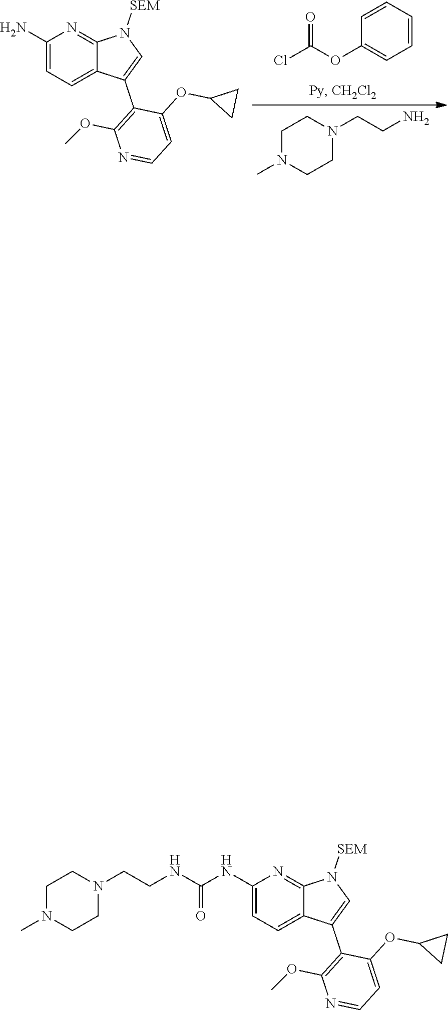

To a solution of 3-(4-cyclopropoxy-2-methoxypyridin-3-yl)-1-((2-(trimethylsilyl)ethoxy)methyl)-1H-pyrrolo[2,3-b]pyridin-6-amine (170.0 mg, 0.40 mmol) in pyridine/DCM (3.0 mL/25.0 mL) was added phenyl chloroformate (124.8 mg, 0.80 mmol) at 0° C. The mixture was stirred at 0° C. for 1 h. The resulting mixture was concentrated under vacuum. To the above residue was added a solution of 2-(4-methylpiperazin-1-yl)ethanamine (285.4 mg, 2.00 mmol) in pyridine (10.0 mL). The resulting mixture was stirred at 60° C. for 2 h. After the reaction was completed, the resulting mixture was concentrated under reduced pressure. The residue was purified by flash column chromatography with CH₂Cl₂/CH₃OH (10/1, v/v) to afford 3-[3-(4-cyclopropoxy-2-methoxypyridin-3-yl)-1-{[2-(trimethylsilyl)ethoxy]methyl}pyrrolo[2,3-b]pyridin-6-yl]-1-[2-(4-methylpiperazin-1-yl)ethyl]urea (110.0 mg, 46%) as a red oil. LCMS (ESI, m/z): [M+H]⁺=596.3.

### Step 2: Synthesis of 1-(3-(4-cyclopropoxy-2-methoxypyridin-3-yl)-1H-pyrrolo[2,3-b]pyridin-6-yl)-3-(2-(4-methylpiperazin-1-yl)ethyl)urea formate (Compound 210)

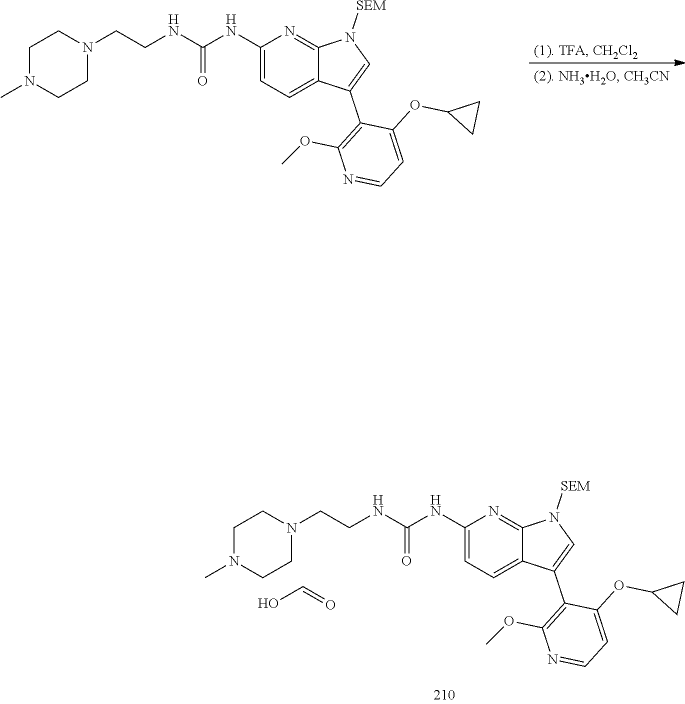

To a solution of 3-[3-(4-cyclopropoxy-2-methoxypyridin-3-yl)-1-{[2-(trimethylsilyl)ethoxy]methyl}pyrrolo[2,3-b]pyridin-6-yl]-1-[2-(4-methylpiperazin-1-yl)ethyl]urea (100.0 mg, 0.168 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in CH₃CN (5.0 mL) was added NH₃·H₂O (5.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue purified by Prep-HPLC with the following conditions (Column: XBridge Prep OBD C18 Column, 30×150 mm, 5 um; Mobile Phase A: Water (0.1% FA), Mobile Phase B: ACN; Flow rate: 60 mL/min; Gradient: 5% B to 25% B in 8 min; 254 nm) to afford 1-(3-(4-cyclopropoxy-2-methoxypyridin-3-yl)-1H-pyrrolo[2,3-b]pyridin-6-yl)-3-(2-(4-methylpiperazin-1-yl)ethyl)urea formate (Compound 210) (55.7 mg, 71%) as a white solid. LCMS (ESI, m/z): [M+H]⁺=466.4. ¹H NMR (400 MHz, Methanol-d₄): δ 8.27 (s, 1H), 8.04 (d, J=5.6 Hz, 1H), 7.59 (d, J=8.8 Hz, 1H), 7.28 (s, 1H), 7.17 (d, J=6.0 Hz, 1H), 6.70 (d, J=8.4 Hz, 1H), 3.93-3.89 (m, 4H), 3.59-3.56 (m, 2H), 3.21-3.17 (m, 4H), 2.92 (s, 3H), 2.83-2.80 (m, 6H), 0.85-0.81 (m, 2H), 0.70-0.65 (m, 2H).

### Example S198: Synthesis of (1S,2S)—N-[3-(2,6-dimethoxyphenyl)-1H-pyrrolo[2,3-b]pyridin-6-yl]-2-[(4-ethylpiperazin-1-yl)methyl]cyclopropane-1-carboxamide and (1R,2R)—N-[3-(2,6-dimethoxyphenyl)-1H-pyrrolo[2,3-b]pyridin-6-yl]-2-[(4-ethylpiperazin-1-yl)methyl]cyclopropane-1-carboxamide (Compound 211 and Compound 212)

### Step 1: Synthesis of trans-N-[3-(2,6-dimethoxyphenyl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide

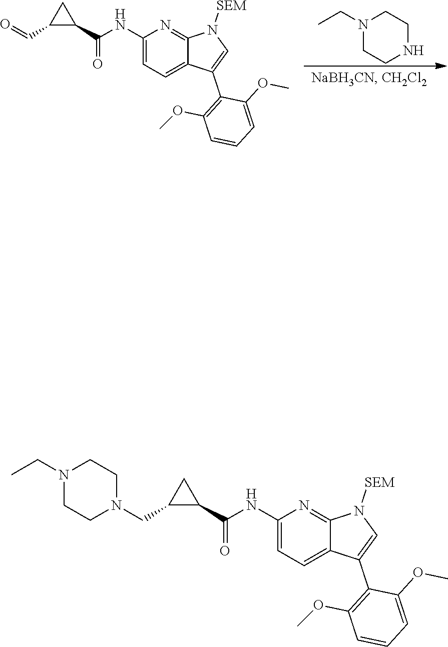

To a solution of trans-N-[3-(2,6-dimethoxyphenyl)-1-{[2-(trimethylsilyl)ethoxy]methyl}pyrrolo[2,3-b]pyridin-6-yl]-2-formylcyclopropane-1-carboxamide (400.0 mg, crude) in CH₂Cl₂ (20.0 mL) was added 1-ethylpiperazine (276.5 mg, 2.42 mmol) and NaBH₃CN (152.1 mg, 2.42 mmol) at room temperature. The resulting mixture was stirred at room temperature for 1 h. After the reaction was completed, the reaction mixture was quenched with MeOH. The resulting mixture was concentrated under reduced pressure. The residue was purified by reverse phase flash column chromatography with CH₃CN/H₂O (8/1, v/v) to afford trans-N-[3-(2,6-dimethoxyphenyl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide (180.0 mg, 38%) as a white solid. LCMS (ESI, m/z): [M+H]⁺=594.3.

### Step 2: Synthesis of trans-N-[3-(2,6-dimethoxyphenyl)-1H-pyrrolo[2,3-b]pyridin-6-yl]-2-[(4-ethylpiperazin-1-yl)methyl]cyclopropane-1-carboxamide

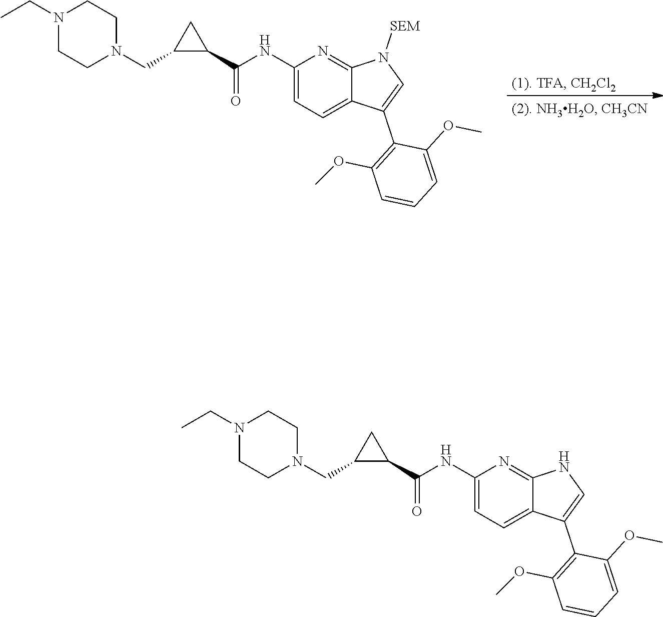

To a solution of trans-N-[3-(2,6-dimethoxyphenyl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide (150.0 mg, 0.26 mmol) in CH₂Cl₂ (5.0 mL) was added TFA (5.0 mL) at room temperature. The resulting mixture was stirred at room temperature for 3 h. The resulting mixture was concentrated under reduced pressure. To the residue in CH₃CN (5.0 mL) was added NH₃·H₂O (5.0 mL) at room temperature. The resulting mixture was stirred at room temperature for 16 h. After the reaction was completed, the resulting mixture was concentrated under reduced pressure. The residue was purified by reverse phase flash chromatography with CH₃OH/H₂O (7/1, v/v) to afford trans-N-[3-(2,6-dimethoxyphenyl)-1H-pyrrolo[2,3-b]pyridin-6-yl]-2-[(4-ethylpiperazin-1-yl)methyl]cyclopropane-1-carboxamide (65.7 mg, 55%) as a white solid. LCMS (ESI, m/z): [M+H]⁺=464.3.

### Step 3: Synthesis of (1S,2S)—N-[3-(2,6-dimethoxyphenyl)-1H-pyrrolo[2,3-b]pyridin-6-yl]-2-[(4-ethylpiperazin-1-yl)methyl]cyclopropane-1-carboxamide and (1R,2R)—N-[3-(2,6-dimethoxyphenyl)-1H-pyrrolo[2,3-b]pyridin-6-yl]-2-[(4-ethylpiperazin-1-yl)methyl]cyclopropane-1-carboxamide (Compound 211 and Compound 212)

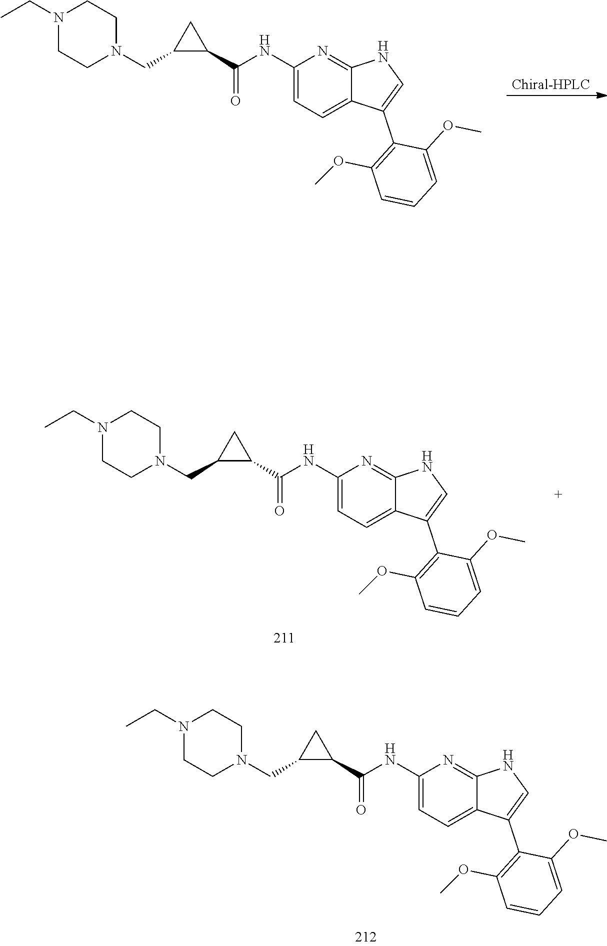

The product trans-N-[3-(2,6-dimethoxyphenyl)-1H-pyrrolo[2,3-b]pyridin-6-yl]-2-[(4-ethylpiperazin-1-yl)methyl]cyclopropane-1-carboxamide (65.7 mg, 0.14 mmol) was separated by Prep-Chiral-HPLC with the following conditions (Column: CHIRALPAK IG, 2×25 cm, 5 m; Mobile Phase A:Hex:DCM=3:1 (0.5% 2M NH₃-MeOH)—HPLC, Mobile Phase B: EtOH—HPLC; Flow rate: 20 mL/min; Gradient: 20% B to 20% B in 14 min; Wave Length: 220/254 nm) to afford N-[3-(2,6-dimethoxyphenyl)-1H-pyrrolo[2,3-b]pyridin-6-yl]-2-[(4-ethylpiperazin-1-yl)methyl]cyclopropane-1-carboxamide Enantiomer 1 (10.7 mg, 16%) as a white solid and N-[3-(2,6-dimethoxyphenyl)-1H-pyrrolo[2,3-b]pyridin-6-yl]-2-[(4-ethylpiperazin-1-yl)methyl]cyclopropane-1-carboxamide Enantiomer 2 (8.5 mg, 13%) as a white solid. The absolute stereochemistry of Enantiomers 1 and 2 was not assigned. The two enantiomeric structures that could be obtained from chiral separation of the enantiomeric mixture as described above are shown as Compounds 211 and 212 in Table 1.

N-[3-(2,6-dimethoxyphenyl)-1H-pyrrolo[2,3-b]pyridin-6-yl]-2-[(4-ethylpiperazin-1-yl)methyl]cyclopropane-1-carboxamide Enantiomer 1: Retention Time 1=7.55 min; LCMS (ESI, m/z): [M+H]⁺=464.3. ¹H NMR (400 MHz, DMSO-d₆): δ 11.40 (d, J=1.6 Hz, 1H), 10.52 (s, 1H), 7.81 (d, J=8.4 Hz, 1H), 7.52 (d, J=8.4 Hz, 1H), 7.29-7.25 (m, 2H), 6.75 (d, J=8.4 Hz, 2H), 3.69 (s, 6H), 2.46-2.21 (m, 10H), 1.91-1.88 (m, 1H), 1.36-1.31 (m, 1H), 1.05-0.97 (m, 4H), 0.78-0.65 (m, 1H).

N-[3-(2,6-dimethoxyphenyl)-1H-pyrrolo[2,3-b]pyridin-6-yl]-2-[(4-ethylpiperazin-1-yl)methyl]cyclopropane-1-carboxamide Enantiomer 2: Retention Time 2=11.05 min; LCMS (ESI, m/z): [M+H]⁺=464.3. ¹H NMR (400 MHz, DMSO-d₆): δ 11.40 (d, J=1.6 Hz, 1H), 10.52 (s, 1H), 7.81 (d, J=8.4 Hz, 1H), 7.52 (d, J=8.4 Hz, 1H), 7.30-7.25 (m, 2H), 6.75 (d, J=8.4 Hz, 2H), 3.69 (s, 6H), 2.46-2.21 (m, 10H), 1.90-1.88 (m, 1H), 1.36-1.31 (m, 1H), 1.05-0.97 (m, 4H), 0.78-0.65 (m, 1H).

### Example S199: Synthesis of (1R,2R)—N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide and (1S,2S)—N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide (Compound 213 and Compound 214)

### Step 1: Synthesis of 5-bromo-4-chloro-6-methoxypyrimidine

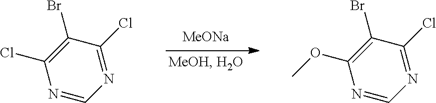

To a solution of 5-bromo-4,6-dichloropyrimidine (30.0 g, 131.64 mmol) in MeOH/H₂O (200.0/200.0 mL) was added MeONa (6.4 g, 118.47 mmol) at room temperature. The resulting mixture was stirred at room temperature for 4 h. After the reaction was completed, the resulting mixture was concentrated under reduced pressure. The residue was diluted with H₂O and extracted with ethyl acetate. The combined organic layer was washed with brine, dried over anhydrous sodium sulfate and filtered. The filtrate was concentrated under reduced pressure to afford 5-bromo-4-chloro-6-methoxypyrimidine (31.0 g, crude) as a white solid. LCMS (ESI, m/z): [M+H]⁺=222.9.

### Step 2: Synthesis of 5-bromo-4-cyclopropoxy-6-methoxypyrimidine

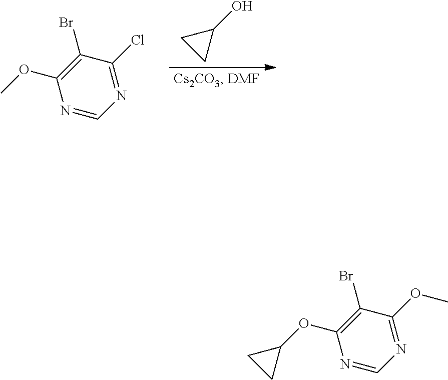

To a solution of 5-bromo-4-chloro-6-methoxypyrimidine (31.0 g, crude) in DMF (120.0 mL) was added Cs₂CO₃ (57.2 g, 175.59 mmol) and cyclopropanol (8.5 g, 146.31 mmol) at room temperature under N₂. The reaction mixture was stirred at 80° C. for 6 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (91/9, v/v) to afford 5-bromo-4-cyclopropoxy-6-methoxypyrimidine (9.0 g, 12%) as a white solid. LCMS (ESI, m/z): [M+H]⁺=245.0.

### Step 3: Synthesis of 5-(6-chloro-1-{[2-(trimethylsilyl)ethoxy]methyl}pyrrolo[2,3-b]pyridin-3-yl)-4-cyclopropoxy-6-methoxypyrimidine

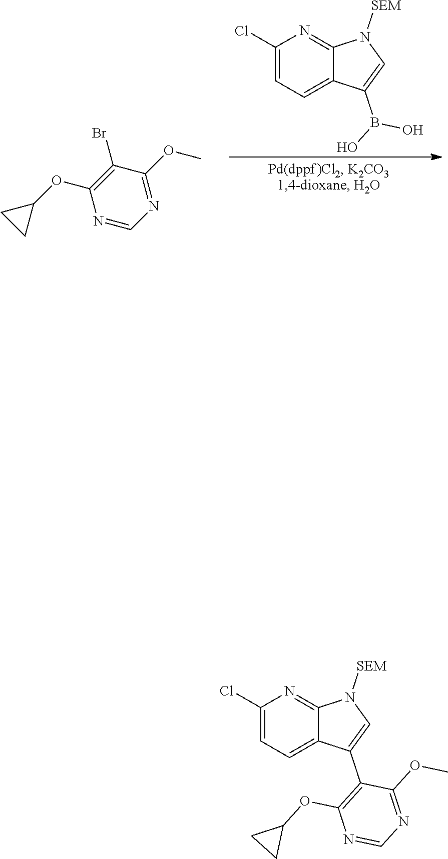

To a solution of 5-bromo-4-cyclopropoxy-6-methoxypyrimidine (8.0 g, 24.48 mmol) in 1.4-dioxane/H₂O (100.0/20.0 mL) was added 6-chloro-1-{[2-(trimethylsilyl)ethoxy]methyl}pyrrolo[2,3-b]pyridin-3-ylboronic acid (12.8 g, 39.17 mmol), K₂CO₃ (13.6 g, 97.92 mmol) and Pd(dppf)Cl₂ (5.3 g, 6.51 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (86/14, v/v) to afford 5-(6-chloro-1-{[2-(trimethylsilyl)ethoxy]methyl}pyrrolo[2,3-b]pyridin-3-yl)-4-cyclopropoxy-6-methoxypyrimidine (5.8 g, 39%) as a yellow oil. LCMS (ESI, m/z): [M+H]⁺=447.2.

### Step 4: Synthesis of Tert-butyl N-[3-(4-cyclopropoxy-6-methoxypyrimidin-5-yl)-1-{[2-(trimethylsilyl)ethoxy]methyl}pyrrolo[2,3-b]pyridin-6-yl]carbamate

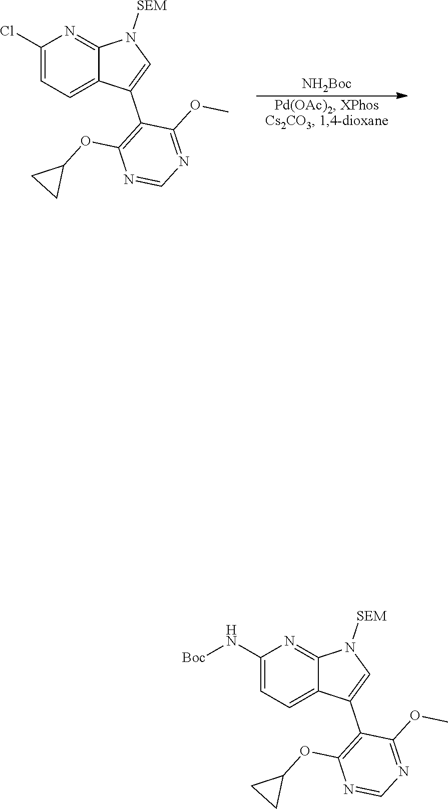 [SMILES: COc1ncnc(OC2CC2)c1-c1cn(COCC[Si](C)(C)C)c2nc(Cl)ccc12 ; "Pd(OAc)2, XPhosCs2CO3, 1,4-dioxane" ; COc1ncnc(OC2CC2)c1-c1cn(COCC[Si](C)(C)C)c2nc(NC(=O)OC(C)(C)C)ccc12 ; "NH2Boc"]

To a solution of 5-(6-chloro-1-{[2-(trimethylsilyl)ethoxy]methyl}pyrrolo[2,3-b]pyridin-3-yl)-4-cyclopropoxy-6-methoxypyrimidine (5.0 g, 11.18 mmol) in 1,4-dioxane (120.0 mL) was added NH₂Boc (6.5 g, 55.82 mmol), Cs₂CO₃ (27.8 g, 33.54 mmol), XPhos (1.7 g, 2.23 mmol) and Pd(OAc)₂ (251.2 mg, 1.11 mmol) at room temperature. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (92/8, v/v) to afford tert-butyl N-[3-(4-cyclopropoxy-6-methoxypyrimidin-5-yl)-1-{[2-(trimethylsilyl)ethoxy]methyl}pyrrolo[2,3-b]pyridin-6-yl]carbamate (5.0 g, 70%) as a yellow oil. LCMS (ESI, m/z): [M+H]⁺=528.3.

### Step 5: Synthesis of 3-(4-cyclopropoxy-6-methoxypyrimidin-5-yl)-1-{[2-(trimethylsilyl)ethoxy]methyl}pyrrolo[2,3-b]pyridin-6-amine

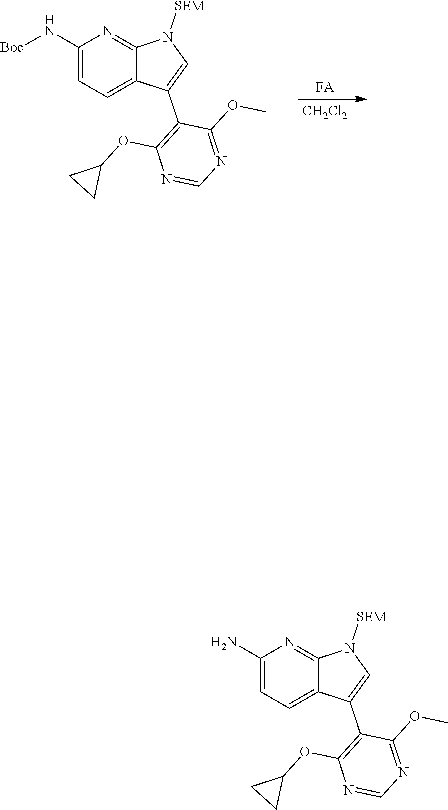

A solution of tert-butyl N-[3-(4-cyclopropoxy-6-methoxypyrimidin-5-yl)-1-{[2-(trimethylsilyl)ethoxy]methyl}pyrrolo[2,3-b]pyridin-6-yl]carbamate (5.0 g, 7.56 mmol) in FA/CH₂Cl₂ (20.0/20.0 mL) was stirred at room temperature for 16 h. After the reaction was completed, the resulting mixture was adjusted pH to 7 with aq. NaHCO₃ and then extracted with CH₂Cl₂. The combined organic layer was washed with brine, dried over anhydrous sodium sulfate and filtered. The filtrate was concentrated under reduced pressure to afford 3-(4-cyclopropoxy-6-methoxypyrimidin-5-yl)-1-{[2-(trimethylsilyl)ethoxy]methyl}pyrrolo[2,3-b]pyridin-6-amine (3.0 g, crude) as a yellow oil. LCMS (ESI, m/z): [M+H]⁺=428.2.

### Step 6: Synthesis of Methyl (trans)-2-{[3-(4-cyclopropoxy-6-methoxypyrimidin-5-yl)-1-{[2-(trimethylsilyl)ethoxy]methyl}pyrrolo[2,3-b]pyridin-6-yl]carbamoyl}cyclopropane-1-carboxylate

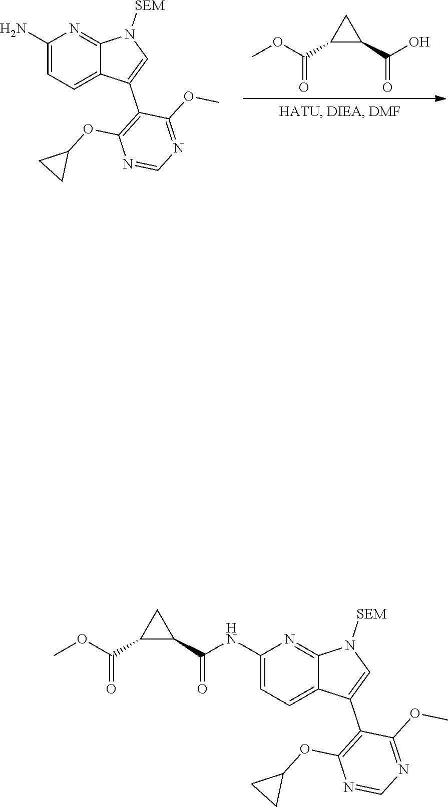

To a solution of 3-(4-cyclopropoxy-6-methoxypyrimidin-5-yl)-1-{[2-(trimethylsilyl)ethoxy]methyl}pyrrolo[2,3-b]pyridin-6-amine (2.0 g, crude) in DMF (40.0 mL) was added trans-2-(methoxycarbonyl)cyclopropane-1-carboxylic acid (2.1 g, 4.54 mmol), DIEA (2.4 g, 18.94 mmol) and HATU (2.2 g, 5.68 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (88/12, v/v) to afford methyl (trans)-2-{[3-(4-cyclopropoxy-6-methoxypyrimidin-5-yl)-1-{[2-(trimethylsilyl)ethoxy]methyl}pyrrolo[2,3-b]pyridin-6-yl]carbamoyl}cyclopropane-1-carboxylate (1.0 g, 47%) as a yellow oil. LCMS (ESI, m/z): [M+H]⁺=554.2.

### Step 7: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide

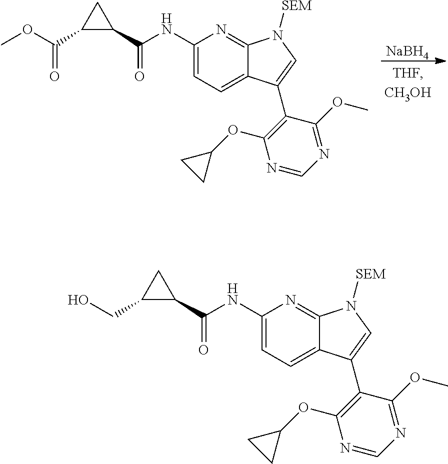

To a solution of methyl (trans)-2-{[3-(4-cyclopropoxy-6-methoxypyrimidin-5-yl)-1-{[2-(trimethylsilyl)ethoxy]methyl}pyrrolo[2,3-b]pyridin-6-yl]carbamoyl}cyclopropane-1-carboxylate (1.0 g, 1.80 mmol) in THF/MeOH (5.0/5.0 mL) was added NaBH₄ (2.1 g, 54.18 mmol) at room temperature. The resulting mixture was stirred at room temperature for 2 h. After the reaction was completed. the resulting mixture was diluted with H₂O and extracted with ethyl acetate. The combined organic layer was washed with brine, dried over anhydrous sodium sulfate and filtered. The filtrate was evaporated in vacuo. The residue was purified by reverse phase flash column chromatography with petroleum CH₃CN/H₂O (60/40, v/v) to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide (560.0 mg, 70%) as a white solid. LCMS (ESI, m/z): [M+H]⁺=526.2.

### Step 8: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide

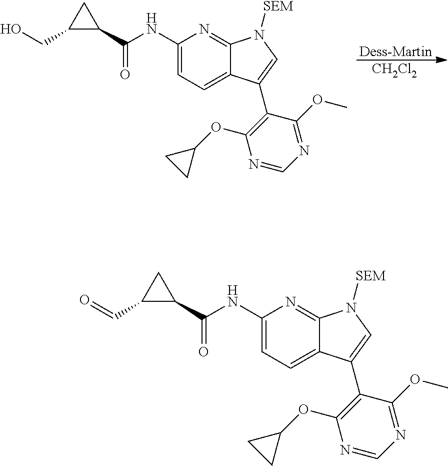 [SMILES: COc1ncnc(OC2CC2)c1-c1cn(COCC[Si](C)(C)C)c2nc(NC(=O)[C@@H]3C[C@H]3CO)ccc12 ; "Dess-Martin" ; "CH2Cl2" ; COc1ncnc(OC2CC2)c1-c1cn(COCC[Si](C)(C)C)c2nc(NC(=O)[C@@H]3C[C@H]3C=O)ccc12]

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-(hydroxymethyl)cyclopropane-1-carboxamide (560.0 mg, 1.23 mmol) in CH₂Cl₂ (8.0 mL) was added Dess-Martin (1.2 g, 3.00 mmol) at room temperature. The resulting mixture was stirred at room temperature for 2 h. After the reaction was completed, the resulting mixture was diluted with H₂O and extracted with CH₂Cl₂. The organic layer was dried over anhydrous sodium sulfate and filtered. The filtrate was concentrated under vacuum to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide (400.0 mg, crude) as a yellow oil. LCMS (ESI, m/z): [M+H]⁺=524.2.

### Step 9: Synthesis of Trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide

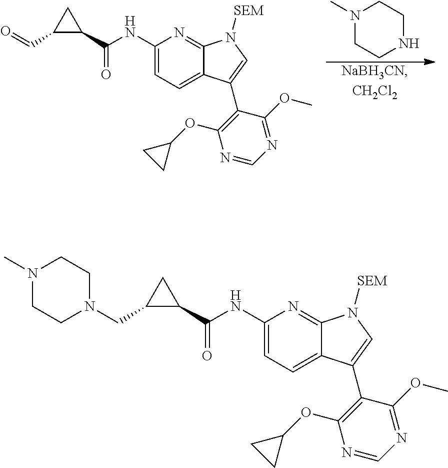

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide (400.0 mg, crude) in CH₂Cl₂ (5.0 mL) was added 1-methylpiperazine (400.0 mg, 3.99 mmol) and NaBH₃CN (150.0 mg, 2.40 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum. The residue was purified by reverse phase flash column chromatography with CH₃CN/H₂O (60/40, v/v) to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (200.0 mg, 62%) as a white solid. LCMS (ESI, m/z): [M+H]⁺=608.3.

### Step 10: Synthesis of Trans-N-[3-(4-cyclopropoxy-6-methoxypyrimidin-5-yl)-1H-pyrrolo[2,3-b]pyridin-6-yl]-2-[(4-methylpiperazin-1-yl)methyl]cyclopropane-1-carboxamide

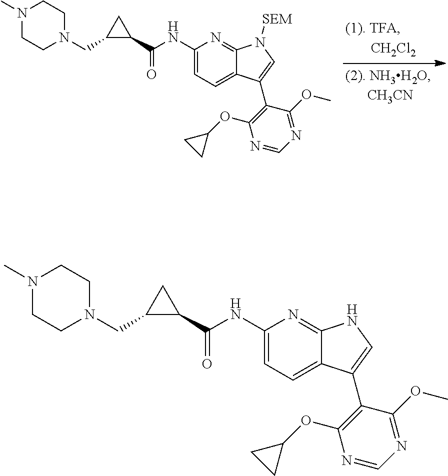

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (200.0 mg, 0.33 mmol) in CH₂Cl₂ (2.0 mL) was added TFA (2.0 mL) at room temperature. The resulting mixture was stirred at room temperature for 3 h. The resulting mixture was concentrated under reduced pressure. To the residue in CH₃CN (2.0 mL) was added NH₃·H₂O (2.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with CH₃CN/H₂O (7/4, v/v) to afford trans-N-[3-(4-cyclopropoxy-6-methoxypyrimidin-5-yl)-1H-pyrrolo[2,3-b]pyridin-6-yl]-2-[(4-methylpiperazin-1-yl)methyl]cyclopropane-1-carboxamide (110.0 mg, 46%) as a white solid. LCMS (ESI, m/z): [M+H]⁺=478.2.

### Step 11: Synthesis of (1R,2R)—N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide and (1S,2S)—N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide (Compound 213 and Compound 214)

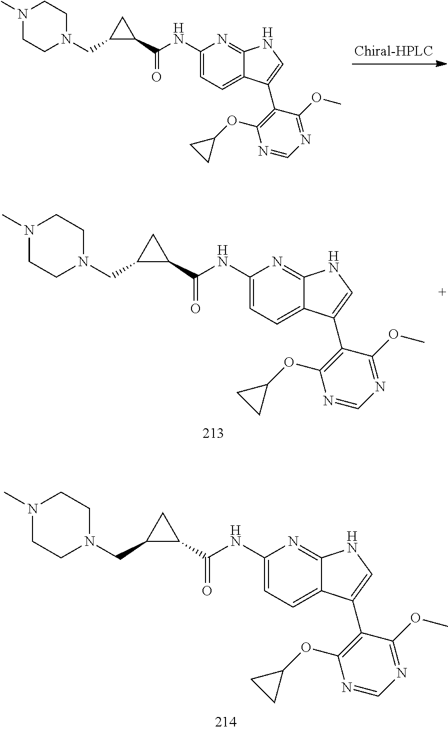

The product trans-N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide (110.0 mg, 0.20 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)—HPLC, Mobile Phase B: EtOH—HPLC; Flow rate: 20 mL/min; Gradient: 30% B to 30% B in 15 min; Wave Length: 220/254 nm) to afford N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide Enantiomer 1 (8.4 mg, 10%) as a white solid and N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide Enantiomer 2 (9.0 mg, 14%) as a white solid. The absolute stereochemistry of Enantiomers 1 and 2 was not assigned. The two enantiomeric structures that could be obtained from chiral separation of the enantiomeric mixture as described above are shown as Compounds 213 and 214 in Table 1.

N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide Enantiomer 1: Retention Time 1=11.427 min; LCMS (ESI, m/z): [M+H]⁺=478.4. ¹H NMR (400 MHz, CD₃OD): δ 8.41 (s, 1H), 7.78-7.70 (m, 2H), 7.43 (s, 1H), 4.42-4.39 (m, 1H), 4.00 (s, 3H), 2.81-2.53 (m, 7H), 2.43-2.23 (m, 5H), 1.83-1.78 (m, 1H), 1.64-1.54 (m, 1H), 1.43-1.21 (m, 2H), 0.98-0.52 (m, 5H).

N-(3-(4-cyclopropoxy-6-methoxypyrimidin-5-yl)-1H-pyrrolo[2,3-b]pyridin-6-yl)-2-((4-methylpiperazin-1-yl)methyl)cyclopropane-1-carboxamide Enantiomer 2: Retention Time 2=13.695 min; LCMS (ESI, m/z): [M+H]⁺=478.4. ¹H NMR (400 MHz, CD₃OD): δ 8.41 (s, 1H), 7.76-7.68 (m, 2H), 7.41 (s, 1H), 4.40-4.37 (m, 1H), 3.98 (s, 3H), 2.80-2.51 (m, 7H), 2.39-2.29 (m, 5H), 1.81-1.76 (m, 1H), 1.62-1.52 (m, 1H), 1.33-1.24 (m, 2H), 0.90-0.66 (m, 5H).

### Example S200: Synthesis of (1S,2S)—N-[3-(4-cyclopropoxy-2-methoxypyridin-3-yl)-1H-pyrazolo[3,4-b]pyridin-6-yl]-2-fluorocyclopropane-1-carboxamide (Compound 215)

### Step 1: Synthesis of 3-(6-chloro-1-{[2-(trimethylsilyl)ethoxy]methyl}pyrazolo[3,4-b]pyridin-3-yl)-4-cyclopropoxy-2-methoxypyridine

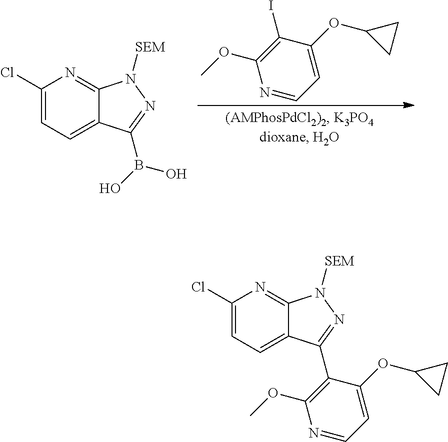 [SMILES: C[Si](C)(C)CCOCn1nc(B(O)O)c2ccc(Cl)nc21 ; COc1nccc(OC2CC2)c1I ; COc1nccc(OC2CC2)c1-c1nn(COCC[Si](C)(C)C)c2nc(Cl)ccc12]

To a solution of 4-cyclopropoxy-3-iodo-2-methoxypyridine (300.0 mg, 1.03 mmol) in dioxane/H₂O (10.0/2.0 mL) was added 6-chloro-1-{[2-(trimethylsilyl)ethoxy]methyl}pyrazolo[3,4-b]pyridin-3-ylboronic acid (337.7 mg, 1.03 mmol), K₃PO₄ (656.3 mg, 3.09 mmol), (AMPhosPdCl₂)₂ (145.9 mg, 0.20 mmol) at room temperature under N₂. The resulting mixture was stirred at 80° C. for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (2/1, v/v) to afford 3-(6-chloro-1-{[2-(trimethylsilyl)ethoxy]methyl}pyrazolo[3,4-b]pyridin-3-yl)-4-cyclopropoxy-2-methoxypyridine (80.0 mg, 16%) as a yellow oil. LCMS (ESI, m/z): [M+H]⁺=447.2.

### Step 2: Synthesis of (1S,2S)—N-[3-(4-cyclopropoxy-2-methoxypyridin-3-yl)-1-{[2-(trimethylsilyl)ethoxy]methyl}pyrazolo[3,4-b]pyridin-6-yl]-2-fluorocyclopropane-1-carboxamide

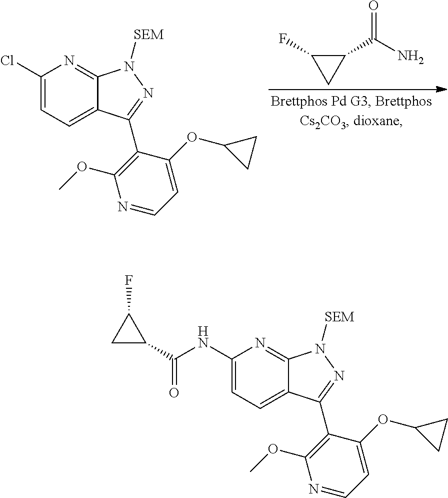 [SMILES: COc1nccc(OC2CC2)c1-c1nn(COCC[Si](C)(C)C)c2nc(Cl)ccc12 ; NC(=O)[C@@H]1C[C@@H]1F ; COc1nccc(OC2CC2)c1-c1nn(COCC[Si](C)(C)C)c2nc(NC(=O)[C@@H]3C[C@@H]3F)ccc12]

To a solution of 3-(6-chloro-1-{[2-(trimethylsilyl)ethoxy]methyl}pyrazolo[3,4-b]pyridin-3-yl)-4-cyclopropoxy-2-methoxypyridine (110.0 mg, 0.25 mmol) in 1,4-dioxane (10.0 mL) was added (1S,2S)-2-fluorocyclopropane-1-carboxamide (126.8 mg, 1.23 mmol), BrettPhos (26.4 mg, 0.05 mmol), Cs₂CO₃ (240.5 mg, 0.74 mmol) and BrettPhos Pd G3 (22.3 mg, 0.03 mmol) at room temperature under N₂. The resulting mixture was stirred at 100° C. for 16 h. After the reaction was completed, the reaction mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford (1S,2S)—N-[3-(4-cyclopropoxy-2-methoxypyridin-3-yl)-1-{[2-(trimethylsilyl)ethoxy]methyl}pyrazolo[3,4-b]pyridin-6-yl]-2-fluorocyclopropane-1-carboxamide (80.0 mg, 63%) as a yellow oil. LCMS (ESI, m/z): [M+H]⁺=514.2.

### Step 3: Synthesis of (1S,2S)—N-[3-(4-cyclopropoxy-2-methoxypyridin-3-yl)-1H-pyrazolo[3,4-b]pyridin-6-yl]-2-fluorocyclopropane-1-carboxamide (Compound 215)

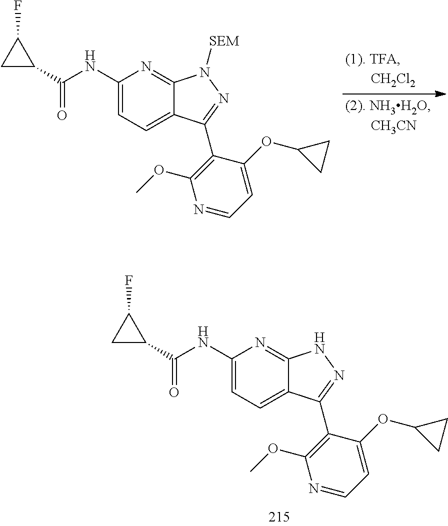 [SMILES: COc1nccc(OC2CC2)c1-c1nn(COCC[Si](C)(C)C)c2nc(NC(=O)[C@@H]3C[C@@H]3F)ccc12 ; COc1nccc(OC2CC2)c1-c1n[nH]c2nc(NC(=O)[C@@H]3C[C@@H]3F)ccc12]

To a solution of (1S,2S)—N-[3-(4-cyclopropoxy-2-methoxypyridin-3-yl)-1-{[2-(trimethylsilyl)ethoxy]methyl}pyrazolo[3,4-b]pyridin-6-yl]-2-fluorocyclopropane-1-carboxamide (70.0 mg, 0.14 mmol) in DCM (4.0 mL) was added TFA (4.0 mL) at room temperature. The resulting mixture was stirred at room temperature for 4 h. The resulting mixture was concentrated under reduced pressure. Then NH₃·H₂O (4.0 mL) and ACN (4.0 mL) were added to the residue at room temperature. The resulting mixture was stirred at room temperature for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column (Xselect CSH OBD Column 30×150 mm, 5 um; Mobile Phase A: Water (10 mmol/L NH₄HCO₃), Mobile Phase B: ACN; Flow rate: 60 mL/min; Gradient: 30% B to 40% B in 8 min; 254 nm) to afford (1S,2S)—N-[3-(4-cyclopropoxy-2-methoxypyridin-3-yl)-1H-pyrazolo[3,4-b]pyridin-6-yl]-2-fluorocyclopropane-1-carboxamide (Compound 215) (12.5 mg, 24%) as a white solid. LCMS (ESI, m/z): [M+H]⁺=384.3. ¹H NMR (400 MHz, DMSO-d₆): δ 13.41 (s, 1H), 10.95 (s, 1H), 8.22 (d, J=5.6 Hz, 1H), 7.94 (d, J=8.8 Hz, 1H), 7.81 (d, J=8.8 Hz, 1H), 7.19 (d, J=5.6 Hz, 1H), 5.06-4.85 (m, 1H), 4.02-3.93 (m, 1H), 3.79 (s, 3H), 2.28-2.25 (m, 1H), 1.71-1.64 (m, 1H), 1.22-1.20 (m, 1H), 0.83-0.74 (m, 2H), 0.59-0.51 (m, 2H).

### Example S201: Synthesis of (1S,2S)—N-(3-(4-cyclopropoxy-2-methoxypyridin-3-yl)-1H-pyrrolo[2,3-b]pyridin-6-yl)-2-((4-methylpiperazin-1-yl)methyl)cyclopropanecarboxamide and (1R,2R)—N-(3-(4-cyclopropoxy-2-methoxypyridin-3-yl)-1H-pyrrolo[2,3-b]pyridin-6-yl)-2-((4-methylpiperazin-1-yl)methyl)cyclopropanecarboxamide (Compound 216 and Compound 217)

### Step 1: Synthesis of 3-(4-cyclopropoxy-2-methoxypyridin-3-yl)-1-{[2-(trimethylsilyl)ethoxy]methyl}pyrrolo[2,3-b]pyridin-6-amine

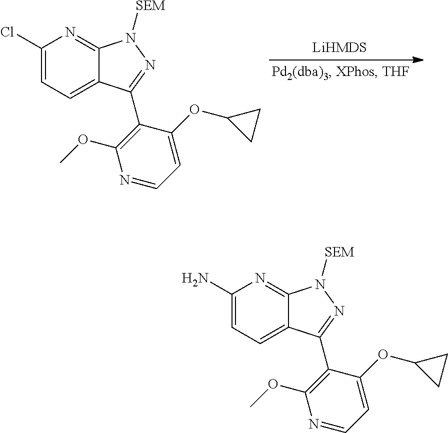 [SMILES: COc1nccc(OC2CC2)c1-c1nn(COCC[Si](C)(C)C)c2nc(Cl)ccc12 ; COc1nccc(OC2CC2)c1-c1nn(COCC[Si](C)(C)C)c2nc(N)ccc12]

To a solution of 3-(6-chloro-1-{[2-(trimethylsilyl)ethoxy]methyl}pyrrolo[2,3-b]pyridin-3-yl)-4-cyclopropoxy-2-methoxypyridine (950.0 mg, 2.13 mmol) in THE (30.0 mL) was added LiHMDS (6.4 mL, 2 mol/L), XPhos (203.1 mg, 0.43 mmol) and Pd₂(dba)₃ (122.5 mg, 0.13 mmol) at room temperature under N₂. The resulting mixture was stirred at 60° C. for 1 h. After the reaction was completed, the reaction mixture was quenched by the addition of sat. NH₄Cl (aq.). The reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 3-(4-cyclopropoxy-2-methoxypyridin-3-yl)-1-{[2-(trimethylsilyl)ethoxy]methyl}pyrrolo[2,3-b]pyridin-6-amine (690.0 mg, 76%) as a yellow oil. LCMS (ESI, m/z): [M+H]⁺=427.2.

### Step 2: Synthesis of Methyl (trans)-2-{[3-(4-cyclopropoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate

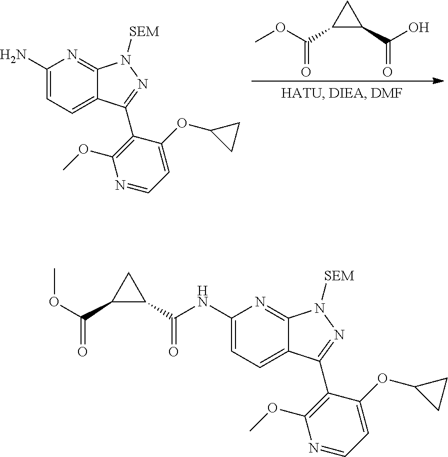

To a solution of 3-(4-cyclopropoxy-2-methoxypyridin-3-yl)-1-{[2-(trimethylsilyl)ethoxy]methyl}pyrrolo[2,3-b]pyridin-6-amine (640.0 mg, 1.50 mmol) in DMF (20.0 mL) was added DIEA (969.5 mg, 7.50 mmol), HATU (855.7 mg, 2.25 mmol) and trans-2-(methoxycarbonyl)cyclopropane-1-carboxylic acid (259.5 mg, 1.80 mmol) at room temperature. The resulting mixture was stirred at room temperature for 4 h. After the reaction was completed, the mixture was purified by reverse phase flash chromatography with ACN/H₂O (2/1, v/v) to afford methyl (trans)-2-{[3-(4-cyclopropoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate (720.0 mg, 87%) as a colorless oil. LCMS (ESI, m/z): [M+H]⁺=553.2.

### Step 3: Synthesis of trans-N-[3-(4-cyclopropoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide

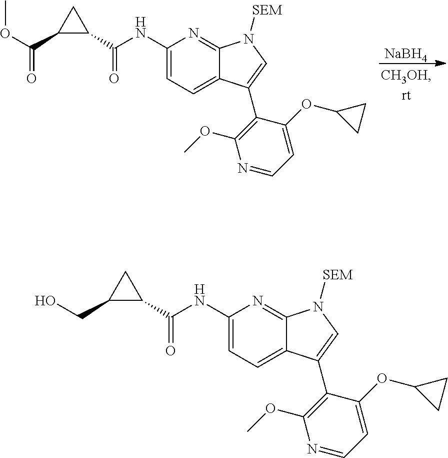 [SMILES: COC(=O)[C@H]1C[C@@H]1C(=O)Nc1ccc2c(-c3c(OC4CC4)ccnc3OC)cn(COCC[Si](C)(C)C)c2n1 ; "NaBH4" ; COc1nccc(OC2CC2)c1-c1cn(COCC[Si](C)(C)C)c2nc(NC(=O)[C@H]3C[C@@H]3CO)ccc12]

To a solution of methyl (trans)-2-{[3-(4-cyclopropoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate (700.0 mg, 1.27 mmol) in THE (6.0 mL)/CH₃OH (4.0 mL) was added NaBH₄ (958.3 mg, 25.34 mmol) at room temperature. The resulting mixture was stirred at 40° C. for 3 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (2/1, v/v) to afford trans-N-[3-(4-cyclopropoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (600.0 mg, 90%) as a colorless oil. LCMS (ESI, m/z): [M+H]⁺=525.2.

### Step 4: Synthesis of trans-N-[3-(4-cyclopropoxy-2-methoxypyridin-3-yl)-1-{[2-(trimethylsilyl)ethoxy]methyl}pyrrolo[2,3-b]pyridin-6-yl]-2-formylcyclopropane-1-carboxamide

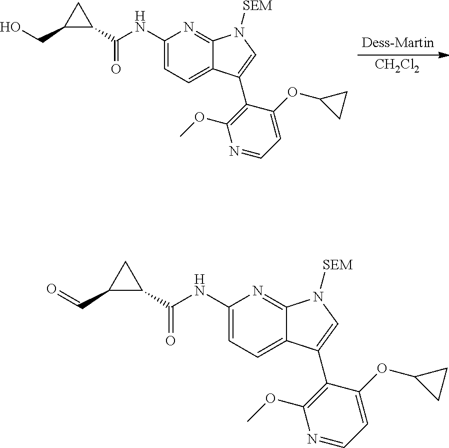

To a solution of trans-N-[3-(4-cyclopropoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (200.0 mg, 0.38 mmol) in DCM (10.0 mL) was added Dess-Martin (242.5 mg, 0.57 mmol) at 0° C. The resulting mixture was stirred at room temperature for 1 h. After the reaction was completed, the reaction mixture was quenched by the addition of sat. NaHCO₃ (aq.). The reaction mixture was diluted with H₂O and extracted with CH₂Cl₂. The combined organic layer was washed with brine, dried over anhydrous sodium sulfate and filtered. The filtrate was concentrated under reduced pressure to afford trans-N-[3-(4-cyclopropoxy-2-methoxypyridin-3-yl)-1-{[2-(trimethylsilyl)ethoxy]methyl}pyrrolo[2,3-b]pyridin-6-yl]-2-formylcyclopropane-1-carboxamide (190.0 mg, crude) as a green oil. LCMS (ESI, m/z): [M+H]⁺=523.2.

### Step 5: Synthesis of trans-N-[3-(4-cyclopropoxy-2-methoxypyridin-3-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide

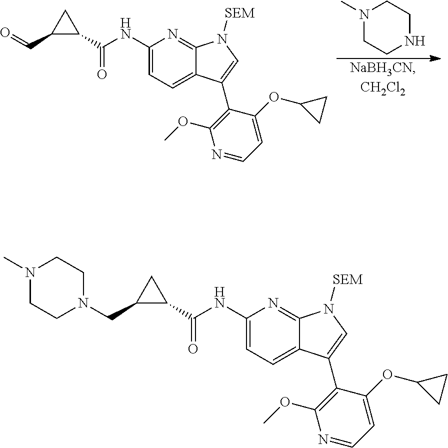

To a solution of trans-N-[3-(4-cyclopropoxy-2-methoxypyridin-3-yl)-1-{[2-(trimethylsilyl)ethoxy]methyl}pyrrolo[2,3-b]pyridin-6-yl]-2-formylcyclopropane-1-carboxamide (190.0 mg, crude) in DCM (6.0 mL) was added 1-methylpiperazine (109.2 mg, 1.09 mmol) and NaBH₃CN (68.5 mg, 1.09 mmol) at room temperature. The resulting mixture was stirred at room temperature for 2 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford trans-N-[3-(4-cyclopropoxy-2-methoxypyridin-3-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (100.0 mg, 45%) as a yellow oil. LCMS (ESI, m/z): [M+H]⁺=607.3.

### Step 6: Synthesis of trans-N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide

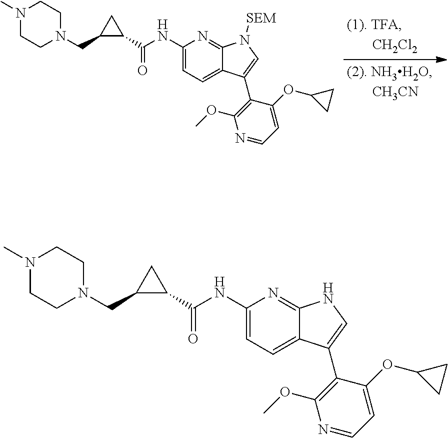 [SMILES: COc1nccc(OC2CC2)c1-c1cn(COCC[Si](C)(C)C)c2nc(NC(=O)[C@H]3C[C@@H]3CN3CCN(C)CC3)ccc12 ; COc1nccc(OC2CC2)c1-c1c[nH]c2nc(NC(=O)[C@H]3C[C@@H]3CN3CCN(C)CC3)ccc12]

To a solution of trans-N-[3-(4-cyclopropoxy-2-methoxypyridin-3-yl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (280.0 mg, 0.46 mmol) in CH₂Cl₂ (10.0 mL) was added TFA (10.0 mL) at room temperature. The resulting mixture was stirred at room temperature for 3 h. The resulting mixture was concentrated under reduced pressure. To the residue in CH₃CN (10.0 mL) was added NH₃·H₂O (10.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (2/1, v/v) to afford trans-N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide (100.0 mg, 45%) as a white solid. LCMS (ESI, m/z): [M+H]⁺=477.3.

### Step 7: Synthesis of (1S,2S)—N-(3-(4-cyclopropoxy-2-methoxypyridin-3-yl)-1H-pyrrolo[2,3-b]pyridin-6-yl)-2-((4-methylpiperazin-1-yl)methyl)cyclopropanecarboxamide and (1R,2R)—N-(3-(4-cyclopropoxy-2-methoxypyridin-3-yl)-1H-pyrrolo[2,3-b]pyridin-6-yl)-2-((4-methylpiperazin-1-yl)methyl)cyclopropanecarboxamide (Compound 216 and Compound 217)

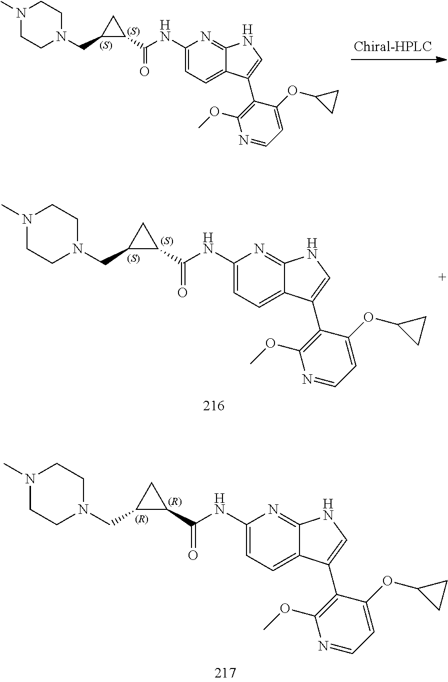

The product trans-N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide (100.0 mg, 0.18 mmol) was separated by Prep-Chiral-HPLC with the following conditions Column: Column (CHIRAL ART Cellulose-SC, 2×25 cm, 5 m; Mobile Phase A: Hex (0.5% 2M NH₃-MeOH)—HPLC, Mobile Phase B: MeOH: EtOH=1:1—HPLC; Flow rate: 20 mL/min; Gradient: 40% B to 40% B in 20 min; Wave Length: 220/254 nm) to afford N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide Enantiomer 1 (26.5 mg, 29%) as a white solid and N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide Enantiomer 2 (20.4 mg, 22%) as a white solid. The absolute stereochemistry of Enantiomers 1 and 2 was not assigned. The two enantiomeric structures that could be obtained from chiral separation of the enantiomeric mixture as described above are shown as Compounds 216 and 217 in Table 1.

N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide Enantiomer 1: Retention Time 1=18.75 min; LCMS (ESI, m/z): [M+H]⁺=477.4. ¹H NMR (400 MHz, DMSO-d₆): δ 11.50 (s, 1H), 10.52 (s, 1H), 8.07 (d, J=6.0 Hz, 1H), 7.83 (d, J=8.8 Hz, 1H), 7.56 (d, J=8.8 Hz, 1H), 7.34 (d, J=2.4 Hz, 1H), 7.14 (d, J=6.0 Hz, 1H), 3.94-3.91 (m, 1H), 3.82 (s, 3H), 2.44-2.22 (m, 7H), 2.14 (s, 3H), 1.90-1.88 (m, 1H), 1.34-1.31 (m, 1H), 1.06-1.03 (m, 1H), 0.79-0.76 (m, 2H), 0.71-0.64 (m, 3H).

N-[3-(4-cyclopropoxy-2-methoxypyridin-3-yl)-1H-pyrrolo[2,3-b]pyridin-6-yl]-2-[(4-methylpiperazin-1-yl)methyl]cyclopropane-1-carboxamide Enantiomer 2 Retention Time 2=23.58 min; LCMS (ESI, m/z): [M+H]⁺=477.4. ¹H NMR (400 MHz, DMSO-d₆): δ 11.50 (s, 1H), 10.52 (s, 1H), 8.07 (d, J=6.0 Hz, 1H), 7.83 (d, J=8.4 Hz, 1H), 7.56 (d, J=8.8 Hz, 1H), 7.34 (d, J=2.4 Hz, 1H), 7.14 (d, J=6.0 Hz, 1H), 3.94-3.91 (m, 1H), 3.82 (s, 3H), 2.45-2.22 (m, 9H), 2.14 (s, 3H), 1.90-1.88 (m, 1H), 1.34-1.31 (m, 1H), 1.06-1.03 (m, 1H), 0.79-0.76 (m, 2H), 0.71-0.64 (m, 3H).

### Example S202: Synthesis of (1R,2R)—N-(3-(4-cyclopropoxy-2-methoxypyridin-3-yl)-1H-pyrrolo[2,3-b]pyridin-6-yl)-2-((dimethylamino)methyl)cyclopropane-1-carboxamide and (1S,2S)—N-(3-(4-cyclopropoxy-2-methoxypyridin-3-yl)-1H-pyrrolo[2,3-b]pyridin-6-yl)-2-((dimethylamino)methyl)cyclopropane-1-carboxamide (Compound 218 and Compound 219)

### Step 1: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide

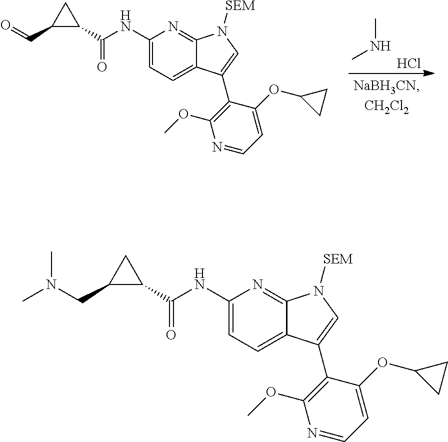

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-formylcyclopropane-1-carboxamide (400.0 mg, crude) in CH₂Cl₂ (5.0 mL) was added dimethylamine hydrochloride (103.5 mg, 1.27 mmol) and NaBH₃CN (144.3 mg, 3.30 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by reverse phase flash column chromatography with acetonitrile/water (16/84, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (168.0 mg, 40%) as a yellow solid. LCMS (ESI, m/z): [M+H]⁺=552.3.

### Step 2: Synthesis of trans-N-(3-(4-cyclopropoxy-2-methoxypyridin-3-yl)-1H-pyrrolo[2,3-b]pyridin-6-yl)-2-((dimethylamino)methyl)cyclopropane-1-carboxamide

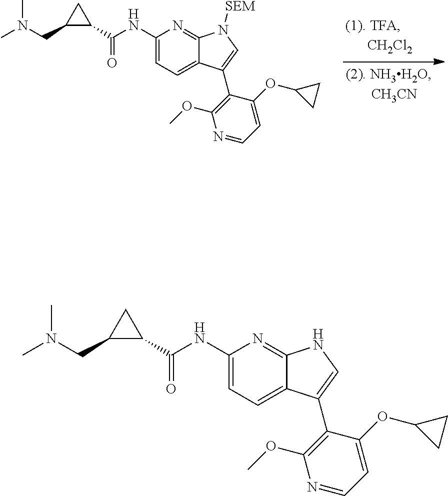 [SMILES: COc1nccc(OC2CC2)c1-c1cn(COCC[Si](C)(C)C)c2nc(NC(=O)[C@H]3C[C@@H]3CN(C)C)ccc12 ; COc1nccc(OC2CC2)c1-c1c[nH]c2nc(NC(=O)[C@H]3C[C@@H]3CN(C)C)ccc12]

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (158.0 mg, 0.29 mmol) in CH₂Cl₂ (5.0 mL) was added TFA (5.0 mL) at room temperature. The resulting mixture was stirred at room temperature for 4 h. The resulting mixture was concentrated under reduced pressure. To the residue in CH₃CN (5.0 mL) was added NH₃·H₂O (5.0 mL) at room temperature. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with acetonitrile/water (65/35, v/v) to afford trans-N-(3-(4-cyclopropoxy-2-methoxypyridin-3-yl)-1H-pyrrolo[2,3-b]pyridin-6-yl)-2-((dimethylamino)methyl)cyclopropane-1-carboxamide (51.0 mg, 41%) as a white solid. LCMS (ESI, m/z): [M+H]⁺=422.4.

### Step 3: Synthesis of (1R,2R)—N-(3-(4-cyclopropoxy-2-methoxypyridin-3-yl)-1H-pyrrolo[2,3-b]pyridin-6-yl)-2-((dimethylamino)methyl)cyclopropane-1-carboxamide and (1S,2S)—N-(3-(4-cyclopropoxy-2-methoxypyridin-3-yl)-1H-pyrrolo[2,3-b]pyridin-6-yl)-2-((dimethylamino)methyl)cyclopropane-1-carboxamide (Compound 218 and Compound 219)

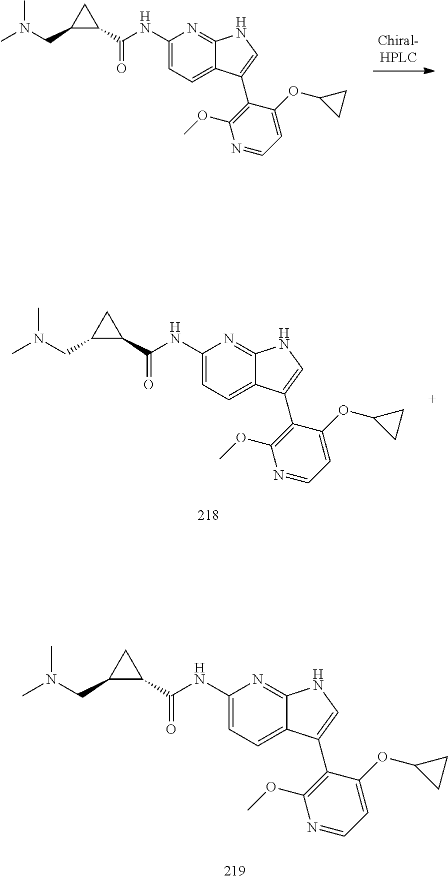 [SMILES: COc1nccc(OC2CC2)c1-c1c[nH]c2nc(NC(=O)[C@H]3C[C@@H]3CN(C)C)ccc12 ; COc1nccc(OC2CC2)c1-c1c[nH]c2nc(NC(=O)[C@@H]3C[C@H]3CN(C)C)ccc12 ; COc1nccc(OC2CC2)c1-c1c[nH]c2nc(NC(=O)[C@H]3C[C@@H]3CN(C)C)ccc12]

The product trans-N-(3-(4-cyclopropoxy-2-methoxypyridin-3-yl)-1H-pyrrolo[2,3-b]pyridin-6-yl)-2-((dimethylamino)methyl)cyclopropane-1-carboxamide (100.0 mg, 0.23 mmol) was separated by Prep-Chiral-HPLC with the following conditions (Column: CHIRAL ART Cellulose-SC, 2×25 cm, 5 um; Mobile Phase A: Hex (0.5% 2M NH₃-MeOH)—HPLC, Mobile Phase B: MeOH:EtOH=1:1—HPLC; Flow rate: 20 mL/min; Gradient: 30% B to 30% B in 14 min; 254 nm) to afford N-(3-(4-cyclopropoxy-2-methoxypyridin-3-yl)-1H-pyrrolo[2,3-b]pyridin-6-yl)-2-((dimethylamino)methyl)cyclopropane-1-carboxamide Enantiomer 1 (11.1 mg, 9%) as a white solid and N-(3-(4-cyclopropoxy-2-methoxypyridin-3-yl)-1H-pyrrolo[2,3-b]pyridin-6-yl)-2-((dimethylamino)methyl)cyclopropane-1-carboxamide Enantiomer 2 (19.1 mg, 16%) as a white solid. The absolute stereochemistry of Enantiomers 1 and 2 was not assigned. The two enantiomeric structures that could be obtained from chiral separation of the enantiomeric mixture as described above are shown as Compounds 218 and 219 in Table 1.

N-(3-(4-cyclopropoxy-2-methoxypyridin-3-yl)-1H-pyrrolo[2,3-b]pyridin-6-yl)-2-((dimethylamino)methyl)cyclopropane-1-carboxamide Enantiomer 1: Retention Time 1=9.93 min; LCMS (ESI, m/z): [M+H]⁺=422.4. ¹H NMR (400 MHz, DMSO-d₆): δ 11.50 (s, 1H), 10.52 (s, 1H), 8.07 (d, J=5.2 Hz, 1H), 7.83 (d, J=8.4 Hz, 1H), 7.56 (d, J=8.8 Hz, 1H), 7.35 (s, 1H), 7.14 (d, J=4.4 Hz, 1H), 3.97-3.91 (m, 1H), 3.82 (s, 3H), 2.41-2.22 (m, 8H), 1.93-1.87 (m, 1H), 1.35-1.24 (m, 1H), 1.10-1.02 (m, 1H), 0.88-0.62 (m, 5H).

N-(3-(4-cyclopropoxy-2-methoxypyridin-3-yl)-1H-pyrrolo[2,3-b]pyridin-6-yl)-2-((dimethylamino)methyl)cyclopropane-1-carboxamide Enantiomer 2: Retention Time 2=12.38 min; LCMS (ESI, m/z): [M+H]⁺=422.3. 1H NMR (400 MHz, DMSO-d₆): δ 11.50 (s, 1H), 10.52 (s, 1H), 8.07 (d, J=5.6 Hz, 1H), 7.82 (d, J=8.4 Hz, 1H), 7.55 (d, J=8.8 Hz, 1H), 7.34 (d, J=2.4 Hz, 1H), 7.13 (d, J=5.6 Hz, 1H), 3.93-3.90 (m, 1H), 3.81 (s, 3H), 2.33-2.16 (m, 8H), 1.91-1.88 (m, 1H), 1.34-1.30 (m, 1H), 1.06-1.02 (m, 1H), 0.84-0.60 (m, 5H).

### Example S203: Synthesis of (1S,2S)—N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide and (1R,2R)—N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide (Compound 220 and Compound 221)

### Step 1: Synthesis of Methyl (trans)-2-((3-(5,7-dimethoxythiazolo[4,5-b]pyridin-6-yl)-1-((2-(trimethylsilyl)ethoxy)methyl)-1H-pyrrolo[2,3-b]pyridin-6-yl)carbamoyl)cyclopropane-1-carboxylate

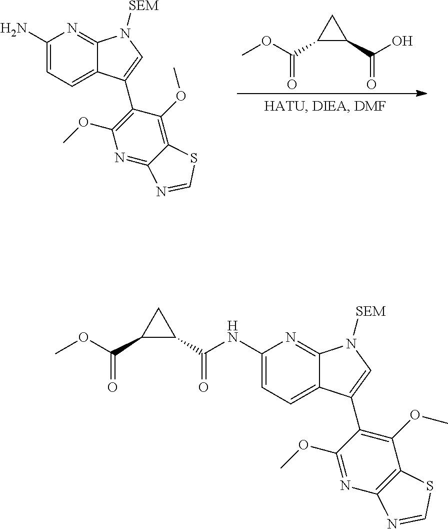

To a solution of 3-(5,7-dimethoxythiazolo[4,5-b]pyridin-6-yl)-1-((2-(trimethylsilyl)ethoxy)methyl)-1H-pyrrolo[2,3-b]pyridin-6-amine (1.9 g, 4.09 mmol) in DMF (20.0 mL) was DIEA (2.6 g, 20.4 mmol), trans-2-(methoxycarbonyl)cyclopropane-1-carboxylic acid (588.9 mg, 4.09 mmol) and HATU (1.9 g, 4.90 mmol) at 0° C. under N₂. The resulting mixture was stirred at room temperature for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (66/34, v/v) to afford methyl (trans)-2-((3-(5,7-dimethoxythiazolo[4,5-b]pyridin-6-yl)-1-((2-(trimethylsilyl)ethoxy)methyl)-1H-pyrrolo[2,3-b]pyridin-6-yl)carbamoyl)cyclopropane-1-carboxylate (1.6 g, 67%) as a light yellow solid. LCMS (ESI, m/z): [M+H]⁺=584.2.

### Step 2: Synthesis of Trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-(hydroxymethyl)cyclopropane-1-carboxamide

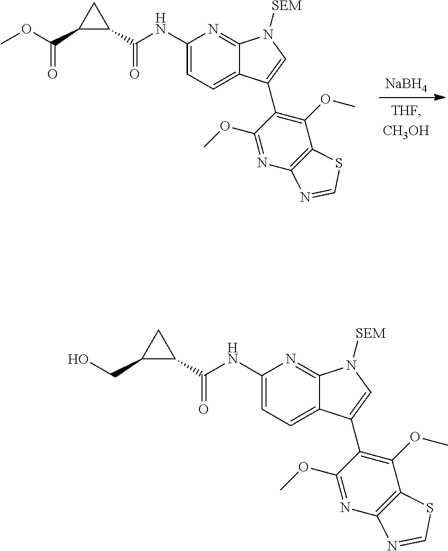

To a solution of methyl (trans)-2-((3-(5,7-dimethoxythiazolo[4,5-b]pyridin-6-yl)-1-((2-(trimethylsilyl)ethoxy)methyl)-1H-pyrrolo[2,3-b]pyridin-6-yl)carbamoyl)cyclopropane-1-carboxylate (1.5 g, 2.57 mmol) in THF/CH₃OH (20.0/5.0 mL) was NaBH₄ (2.9 g, 77.2 mmol) at room temperature. The resulting mixture was stirred at 40° C. for 16 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (60/40, v/v) to afford trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-(hydroxymethyl)cyclopropane-1-carboxamide (800.0 mg, 50%) as a white solid. LCMS (ESI, m/z): [M+H]⁺=556.2.

### Step 3: Synthesis of Trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-formylcyclopropane-1-carboxamide

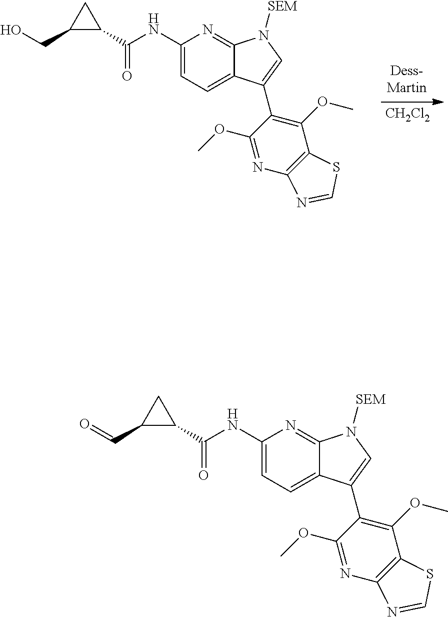

To a solution of trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-(hydroxymethyl)cyclopropane-1-carboxamide (660.0 mg, 1.19 mmol) in CH₂Cl₂ (15.0 mL) was Dess-Martin (755.6 mg, 1.78 mmol) at room temperature. The resulting mixture was stirred at room temperature for 2 h. After the reaction was completed, the resulting mixture was diluted with H₂O and extracted with CH₂Cl₂. The combined organic layer was washed with aq. NaHCO₃, brine, dried over anhydrous sodium sulfate and filtered. The filtrate was concentrated under reduced pressure to afford trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-formylcyclopropane-1-carboxamide (850.0 mg, crude) as a brown solid. LCMS (ESI, m/z): [M+H]⁺=554.2.

### Step 4: Synthesis of Trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide

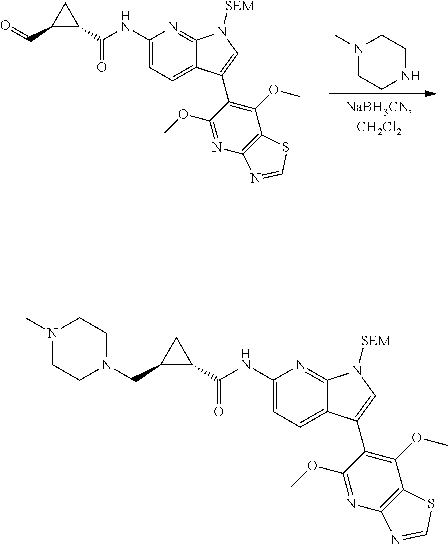

To a solution of trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-formylcyclopropane-1-carboxamide (850.0 mg, 1.54 mmol) in CH₂Cl₂ (20.0 mL) was added 1-methylpiperazine (307.5 mg, 3.07 mmol) and NaBH₃CN (192.9 mg, 3.07 mmol) at room temperature. The resulting mixture was stirred at room temperature for 2 h. After the reaction was completed, the resulting mixture was quenched with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide (170.0 mg, 17%) as a brown solid. LCMS (ESI, m/z): [M+H]⁺=638.3.

### Step 5: Synthesis of Trans-N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide

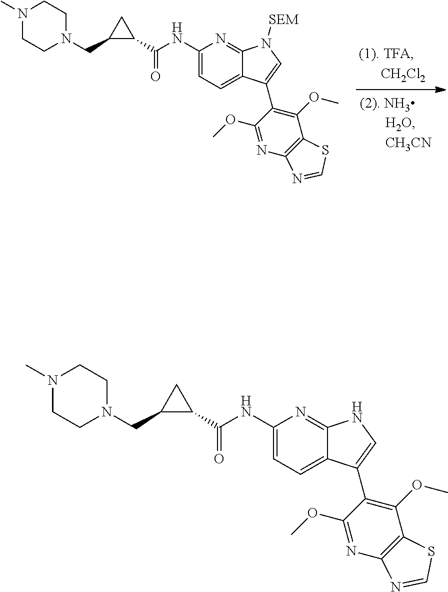

To a solution of trans-N-(3-(5,7-dimethoxythiazolo[4,5-b]pyridin-6-yl)-1-((2-(trimethylsilyl)ethoxy)methyl)-1H-pyrrolo[2,3-b]pyridin-6-yl)-2-((4-methylpiperazin-1-yl)methyl)cyclopropane-1-carboxamide (170.0 mg, 0.27 mmol) in CH₂Cl₂ (2.0 mL) was added TFA (2.0 mL) at room temperature. The resulting mixture was stirred at room temperature for 2 h. The resulting mixture was concentrated under reduced pressure. To the residue in CH₃CN (2.0 mL) was added NH₃·H₂O (2.0 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford trans-N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide (100.0 mg, 70%) as a light yellow solid. LCMS (ESI, m/z): [M+H]⁺=508.2.

### Step 6: Synthesis of (1S,2S)—N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide and (1R,2R)—N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide (Compound 220 and Compound 221)

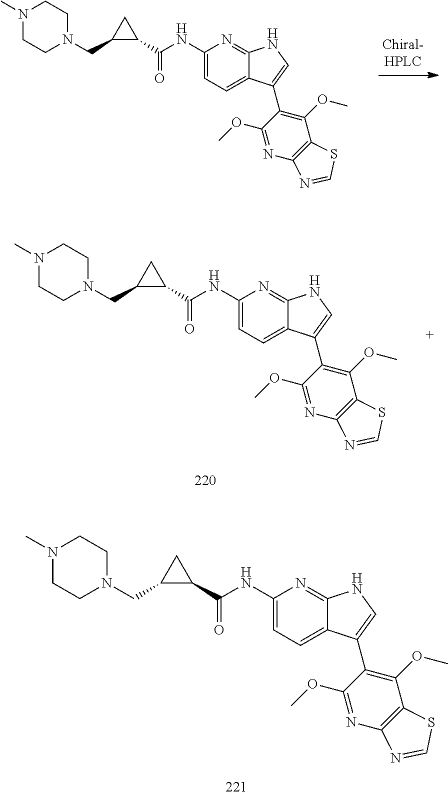

The product trans-N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide (100.0 mg, 0.20 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)—HPLC, Mobile Phase B: MeOH: EtOH=1:1—HPLC; Flow rate: 20 mL/min; Gradient: 50% B to 50% B in 14.5 min; Wave Length: 220/254 nm) to afford N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide Enantiomer 1 (20.7 mg, 41%) as a white solid and N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide Enantiomer 2 (19.7 mg, 39%) as a white solid. The absolute stereochemistry of Enantiomers 1 and 2 was not assigned. The two enantiomeric structures that could be obtained from chiral separation of the enantiomeric mixture as described above are shown as Compounds 220 and 221 in Table 1.

N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide Enantiomer 1: Retention Time 1: 10.25 min; LCMS (ESI, m/z): [M+H]⁺=508.4. ¹H NMR (400 MHz, DMSO-d₆): δ 11.65 (s, 1H), 10.57 (s, 1H), 9.57 (d, J=5.6 Hz, 1H), 7.86 (d, J=8.4 Hz, 1H), 7.69 (d, J=8.4 Hz, 1H), 7.47 (s, 1H), 3.91 (s, 6H), 2.44-2.11 (m, 9H), 2.10-2.01 (m, 4H), 1.91-1.89 (m, 1H), 1.37-1.33 (m, 1H), 1.11-1.00 (m, 1H), 0.77-0.64 (m, 1H).

N-(3-(5,7-dimethoxythiazolo[4,5-b]pyridin-6-yl)-1H-pyrrolo[2,3-b]pyridin-6-yl)-2-((4-methylpiperazin-1-yl)methyl)cyclopropane-1-carboxamide Enantiomer 2 Retention Time 2:12.63 min; LCMS (ESI, m/z): [M+H]⁺=508.4. ¹H NMR (400 MHz, DMSO-d₆): δ 11.65 (s, 1H), 10.57 (s, 1H), 9.56 (s, 1H), 7.86 (d, J=8.4 Hz, 1H), 7.68 (d, J=8.8 Hz, 1H), 7.47 (s, 1H), 3.91 (s, 6H), 2.44-2.20 (m, 10H), 2.14 (s, 3H), 1.91-1.89 (m, 1H), 1.35-1.32 (m, 1H), 1.11-1.00 (m, 1H), 0.77-0.64 (m, 1H).

### Example S204: Synthesis of (1S,2S)—N-[3-(2-fluoro-6-methoxyphenyl)-1H-pyrrolo[2,3-b]pyridin-6-yl]-2-[(4-methylpiperazin-1-yl)methyl]cyclopropane-1-carboxamide and (1R,2R)—N-[3-(2-fluoro-6-methoxyphenyl)-1H-pyrrolo[2,3-b]pyridin-6-yl]-2-[(4-methylpiperazin-1-yl)methyl]cyclopropane-1-carboxamide (Compound 222 and Compound 223)

### Step 1: Synthesis of 6-chloro-3-(2-fluoro-6-methoxyphenyl)-1-{[2-(trimethylsilyl)ethoxy]methyl}pyrrolo[2,3-b]pyridine

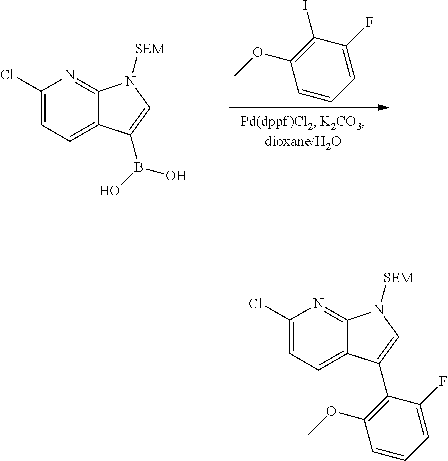

To a solution of 6-chloro-1-{[2-(trimethylsilyl)ethoxy]methyl}pyrrolo[2,3-b]pyridin-3-ylboronic acid (2.0 g, 6.12 mmol) in 1,4-dioxane/H₂O (40.0 mL/8.0 mL) was added 1-fluoro-2-iodo-3-methoxybenzene (1.5 g, 6.12 mmol), K₂CO₃ (1.7 g, 12.25 mmol) and Pd(dppf)Cl₂ (448.0 mg, 0.61 mmol) at room temperature under N₂. The resulting mixture was stirred at 80° C. for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford 6-chloro-3-(2-fluoro-6-methoxyphenyl)-1-{[2-(trimethylsilyl)ethoxy]methyl}pyrrolo[2,3-b]pyridine (2.3 g, 92%) as a brown oil. LCMS (ESI, m/z): [M+H]⁺=407.1.

### Step 2: Synthesis of 3-(2-fluoro-6-methoxyphenyl)-1-{[2-(trimethylsilyl)ethoxy]methyl}pyrrolo[2,3-b]pyridin-6-amine

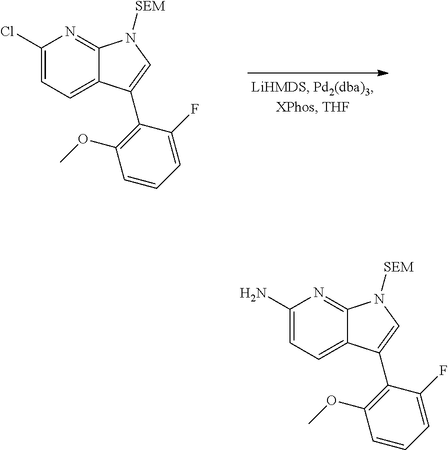 [SMILES: COc1cccc(F)c1-c1cn(COCC[Si](C)(C)C)c2nc(Cl)ccc12 ; COc1cccc(F)c1-c1cn(COCC[Si](C)(C)C)c2nc(N)ccc12]

To a solution of 6-chloro-3-(2-fluoro-6-methoxyphenyl)-1-{[2-(trimethylsilyl)ethoxy]methyl}pyrrolo[2,3-b]pyridine (1.0 g, 2.46 mmol) in THE (10.0 mL) was added XPhos (351.4 mg, 0.74 mmol), Pd₂(dba)₃ (225.0 mg, 0.25 mmol) and LiHMDS (7.4 mL, 1 mol/L) at room temperature under N₂. The resulting mixture was stirred at 60° C. for 2 h. After the reaction was completed, the reaction mixture was quenched by the addition of sat. NH₄Cl (aq.) at room temperature. The resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (4/1, v/v) to afford 3-(2-fluoro-6-methoxyphenyl)-1-{[2-(trimethylsilyl)ethoxy]methyl}pyrrolo[2,3-b]pyridin-6-amine (790.0 mg, 82%) as a brown oil. LCMS (ESI, m/z): [M+H]⁺=388.2.

### Step 3: Synthesis of methyl trans-2-{[3-(2-fluoro-6-methoxyphenyl)-1-{[2-(trimethylsilyl)ethoxy]methyl}pyrrolo[2,3-b]pyridin-6-yl]carbamoyl}cyclopropane-1-carboxylate

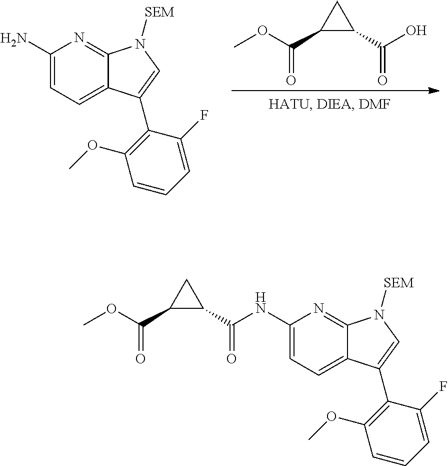

To a solution of 3-(2-fluoro-6-methoxyphenyl)-1-{[2-(trimethylsilyl)ethoxy]methyl}pyrrolo[2,3-b]pyridin-6-amine (770.0 mg, 1.99 mmol) in DMF (20.0 mL) was added DIEA (1.2 g, 9.94 mmol), trans-2-(methoxycarbonyl)cyclopropane-1-carboxylic acid (429.6 mg, 2.98 mmol) and HATU (1.4 g, 3.58 mmol) at room temperature. The resulting mixture was stirred at room temperature for 1 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by reverse phase flash chromatography with acetonitrile/water (4/1, v/v) to afford methyl trans-2-{[3-(2-fluoro-6-methoxyphenyl)-1-{[2-(trimethylsilyl)ethoxy]methyl}pyrrolo[2,3-b]pyridin-6-yl]carbamoyl}cyclopropane-1-carboxylate (760.0 mg, 74%) as a light brown oil. LCMS (ESI, m/z): [M+H]⁺=514.2.

### Step 4: Synthesis of trans-N-[3-(2-fluoro-6-methoxyphenyl)-1-{[2-(trimethylsilyl)ethoxy]methyl}pyrrolo[2,3-b]pyridin-6-yl]-2-(hydroxymethyl)cyclopropane-1-carboxamide

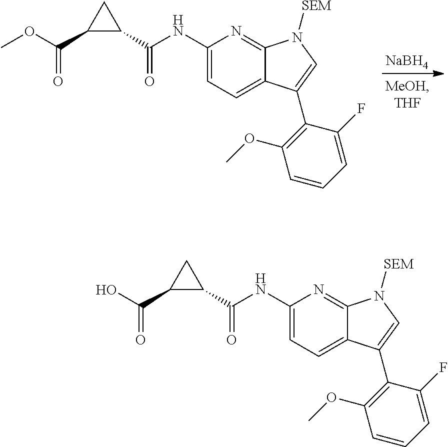 [SMILES: COC(=O)[C@H]1C[C@@H]1C(=O)Nc1ccc2c(-c3c(F)cccc3OC)cn(COCC[Si](C)(C)C)c2n1 ; "NaBH4" ; COc1cccc(F)c1-c1cn(COCC[Si](C)(C)C)c2nc(NC(=O)[C@H]3C[C@@H]3C(=O)O)ccc12]

To a solution of methyl trans-2-{[3-(2-fluoro-6-methoxyphenyl)-1-{[2-(trimethylsilyl)ethoxy]methyl}pyrrolo[2,3-b]pyridin-6-yl]carbamoyl}cyclopropane-1-carboxylate (750.0 mg, 1.46 mmol) in THF/MeOH (12.0 mL/8.0 mL) was added NaBH₄ (1.7 g, 43.8 mmol) at room temperature. The resulting mixture was stirred at 40° C. for 0.5 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by reverse phase flash chromatography with acetonitrile/water (7/3, v/v) to afford trans-N-[3-(2-fluoro-6-methoxyphenyl)-1-{[2-(trimethylsilyl)ethoxy]methyl}pyrrolo[2,3-b]pyridin-6-yl]-2-(hydroxymethyl)cyclopropane-1-carboxamide (365.0 mg, 51%) as a white solid. LCMS (ESI, m/z): [M+H]⁺=486.2.

### Step 5: Synthesis of trans-N-[3-(2-fluoro-6-methoxyphenyl)-1-{[2-(trimethylsilyl)ethoxy]methyl}pyrrolo[2,3-b]pyridin-6-yl]-2-formylcyclopropane-1-carboxamide

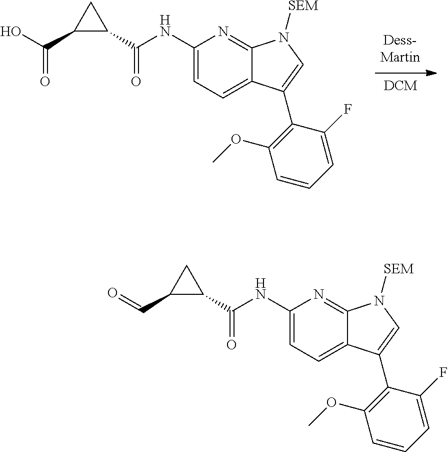 [SMILES: COc1cccc(F)c1-c1cn(COCC[Si](C)(C)C)c2nc(NC(=O)[C@H]3C[C@@H]3C(=O)O)ccc12 ; COc1cccc(F)c1-c1cn(COCC[Si](C)(C)C)c2nc(NC(=O)[C@H]3C[C@@H]3C=O)ccc12]

To a solution of trans-N-[3-(2-fluoro-6-methoxyphenyl)-1-{[2-(trimethylsilyl)ethoxy]methyl}pyrrolo[2,3-b]pyridin-6-yl]-2-(hydroxymethyl)cyclopropane-1-carboxamide (340.0 mg, 0.70 mmol) in CH₂Cl₂ (10.0 mL) was added Dess-Martin (445.4 mg, 1.05 mmol) at 0° C. The resulting mixture was stirred at room temperature for 2 h. After the reaction was completed. the resulting mixture was diluted with water and extracted with CH₂Cl₂. The combined organic layer was washed with brine, dried over anhydrous sodium sulfate and filtered. The filtrate was concentrated under reduced pressure to afford trans-N-[3-(2-fluoro-6-methoxyphenyl)-1-{[2-(trimethylsilyl)ethoxy]methyl}pyrrolo[2,3-b]pyridin-6-yl]-2-formylcyclopropane-1-carboxamide (330.0 mg, crude) as a green oil. LCMS (ESI, m/z): [M+H]⁺=484.2.

### Step 6: Synthesis of trans-N-[3-(2-fluoro-6-methoxyphenyl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide

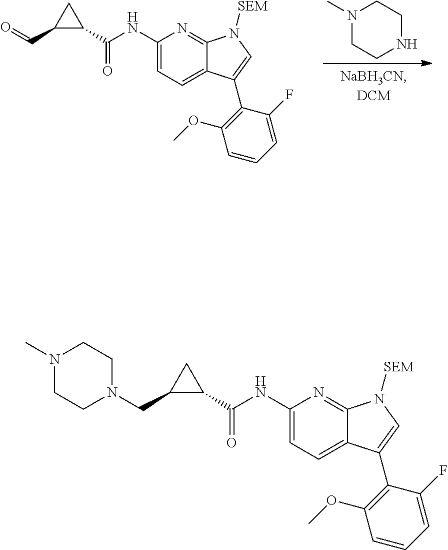

To a solution of trans-N-[3-(2-fluoro-6-methoxyphenyl)-1-{[2-(trimethylsilyl)ethoxy]methyl}pyrrolo[2,3-b]pyridin-6-yl]-2-formylcyclopropane-1-carboxamide (650.0 mg, crude) in DCM (10.0 mL) was added 1-methylpiperazine (403.9 mg, 4.03 mmol) and NaBH₃CN (253.4 mg, 4.03 mmol) at room temperature. The resulting mixture was stirred at room temperature for 2 h. After the reaction was completed, the resulting mixture was diluted with water and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (9/1, v/v) to afford trans-N-[3-(2-fluoro-6-methoxyphenyl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (150.0 mg, 19%) as a light brown oil. LCMS (ESI, m/z): [M+H]⁺=568.3.

### Step 7: Synthesis of trans-N-[3-(2-fluoro-6-methoxyphenyl)-1H-pyrrolo[2,3-b]pyridin-6-yl]-2-[(4-methylpiperazin-1-yl)methyl]cyclopropane-1-carboxamide

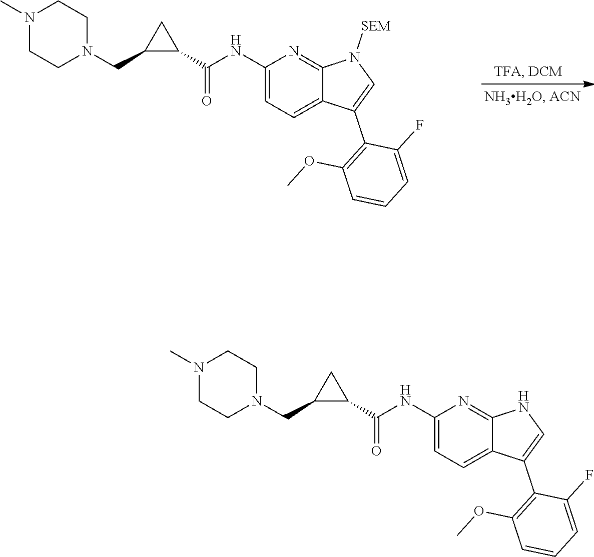 [SMILES: COc1cccc(F)c1-c1cn(COCC[Si](C)(C)C)c2nc(NC(=O)[C@H]3C[C@@H]3CN3CCN(C)CC3)ccc12 ; COc1cccc(F)c1-c1c[nH]c2nc(NC(=O)[C@H]3C[C@@H]3CN3CCN(C)CC3)ccc12]

To a solution of trans-N-[3-(2-fluoro-6-methoxyphenyl)-1-{[2-(trimethylsilyl)ethoxy]methyl}pyrrolo[2,3-b]pyridin-6-yl]-2-[(4-methylpiperazin-1-yl)methyl]cyclopropane-1-carboxamide (140.0 mg, 0.25 mmol) in CH₂Cl₂ (6.0 mL) was added TFA (6.0 mL) at room temperature. The resulting mixture was stirred at room temperature for 3 h. The resulting mixture was concentrated under reduced pressure. To the residue in CH₃CN (6.0 mL) was added NH₃·H₂O (6.0 mL) at room temperature. The resulting mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by reverse phase flash chromatography with acetonitrile/water (1/4, v/v) to afford trans-N-[3-(2-fluoro-6-methoxyphenyl)-1H-pyrrolo[2,3-b]pyridin-6-yl]-2-[(4-methylpiperazin-1-yl)methyl]cyclopropane-1-carboxamide (80.0 mg, 74%) as a light yellow solid. LCMS (ESI, m/z): [M+H]⁺=438.2.

### Step 8: Synthesis of (1S,2S)—N-[3-(2-fluoro-6-methoxyphenyl)-1H-pyrrolo[2,3-b]pyridin-6-yl]-2-[(4-methylpiperazin-1-yl)methyl]cyclopropane-1-carboxamide and (1R,2R)—N-[3-(2-fluoro-6-methoxyphenyl)-1H-pyrrolo[2,3-b]pyridin-6-yl]-2-[(4-methylpiperazin-1-yl)methyl]cyclopropane-1-carboxamide (Compound 222 and Compound 223)

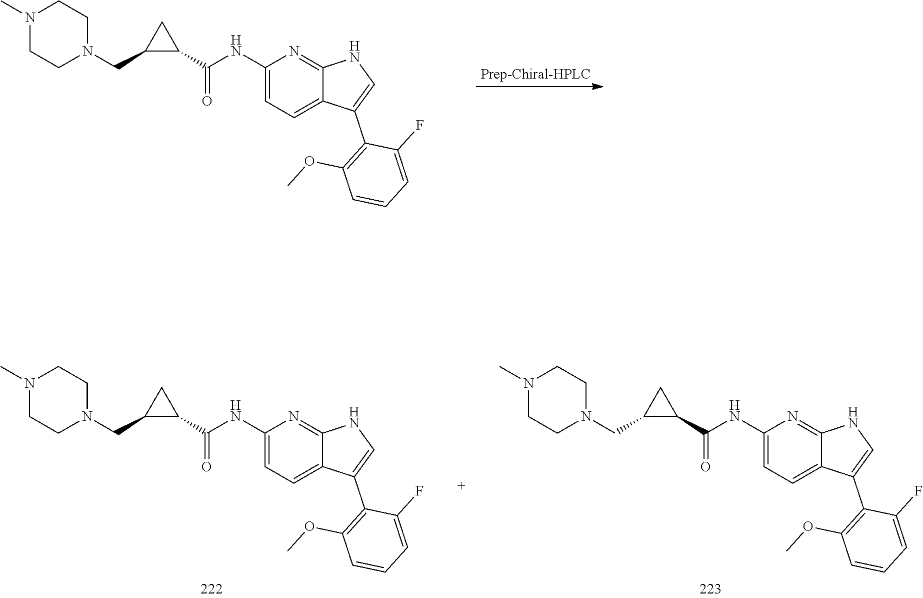 [SMILES: COc1cccc(F)c1-c1c[nH]c2nc(NC(=O)[C@H]3C[C@@H]3CN3CCN(C)CC3)ccc12 ; COc1cccc(F)c1-c1c[nH]c2nc(NC(=O)[C@H]3C[C@@H]3CN3CCN(C)CC3)ccc12 ; COc1cccc(F)c1-c1c[nH]c2nc(NC(=O)[C@@H]3C[C@H]3CN3CCN(C)CC3)ccc12]

The product trans-N-[3-(2-fluoro-6-methoxyphenyl)-1H-pyrrolo[2,3-b]pyridin-6-yl]-2-[(4-methylpiperazin-1-yl)methyl]cyclopropane-1-carboxamide (80.0 mg, 0.18 mmol) was separated by Prep-Chiral-HPLC with the following conditions (Column: CHIRALPAK IE, 2×25 cm, 5 m; Mobile Phase A: Hex (0.5% 2M NH₃-MeOH)—HPLC, Mobile Phase B: MeOH: EtOH=1: 1—HPLC; Flow rate: 16 mL/min; Gradient: 60% B to 60% B in 29 min; Wave Length: 220/254 nm) to afford N-[3-(2-fluoro-6-methoxyphenyl)-1H-pyrrolo[2,3-b]pyridin-6-yl]-2-[(4-methylpiperazin-1-yl)methyl]cyclopropane-1-carboxamide Enantiomer 1 (13.2 mg, 15%) as a white solid and N-[3-(2-fluoro-6-methoxyphenyl)-1H-pyrrolo[2,3-b]pyridin-6-yl]-2-[(4-methylpiperazin-1-yl)methyl]cyclopropane-1-carboxamide Enantiomer 2 (12.3 mg, 15%) as a white solid. The absolute stereochemistry of Enantiomers 1 and 2 was not assigned. The two enantiomeric structures that could be obtained from chiral separation of the enantiomeric mixture as described above are shown as Compounds 222 and 223 in Table 1.

N-[3-(2-fluoro-6-methoxyphenyl)-1H-pyrrolo[2,3-b]pyridin-6-yl]-2-[(4-methylpiperazin-1-yl)methyl]cyclopropane-1-carboxamide Enantiomer 1: Retention Time 1=15.57 min; LCMS (ESI, m/z): [M+H]⁺=438.4. ¹H NMR (400 MHz, DMSO-d₆): δ 11.63 (s, 1H), 10.55 (s, 1H), 7.87 (d, J=8.4 Hz, 1H), 7.66 (d, J=8.4 Hz, 1H), 7.44 (s, 1H), 7.36-7.30 (m, 1H), 6.98-6.89 (m, 2H), 3.78 (s, 3H), 2.45-2.22 (m, 8H), 2.14 (s, 3H), 1.95-1.89 (m, 1H), 1.36-1.32 (m, 1H), 1.06-1.04 (m, 1H), 0.74-0.66 (m, 1H).

N-[3-(2-fluoro-6-methoxyphenyl)-1H-pyrrolo[2,3-b]pyridin-6-yl]-2-[(4-methylpiperazin-1-yl)methyl]cyclopropane-1-carboxamide Enantiomer 2: Retention Time 2=22.25 min; LCMS (ESI, m/z): [M+H]⁺=438.4. ¹H NMR (400 MHz, DMSO-d₆): δ 11.63 (s, 1H), 10.55 (s, 1H), 7.87 (d, J=8.4 Hz, 1H), 7.66 (d, J=8.8 Hz, 1H), 7.44 (s, 1H), 7.36-7.30 (m, 1H), 6.98-6.89 (m, 2H), 3.78 (s, 3H), 2.38-2.22 (m, 8H), 2.14 (s, 3H), 1.94-1.89 (m, 1H), 1.38-1.32 (m, 1H), 1.06-1.01 (m, 1H), 0.72-0.66 (m, 1H).

### Example S205: Synthesis of (1S,2R)—N-(3-(2,6-dimethoxyphenyl)-1H-pyrrolo[2,3-b]pyridin-6-yl)-2-(2-(dimethylamino)ethyl)cyclopropane-1-carboxamide and (1R,2S)—N-(3-(2,6-dimethoxyphenyl)-1H-pyrrolo[2,3-b]pyridin-6-yl)-2-(2-(dimethylamino)ethyl)cyclopropane-1-carboxamide (Compound 224 and Compound 225)

### Step 1: Synthesis of Trans-N-(3-(2,6-dimethoxyphenyl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide

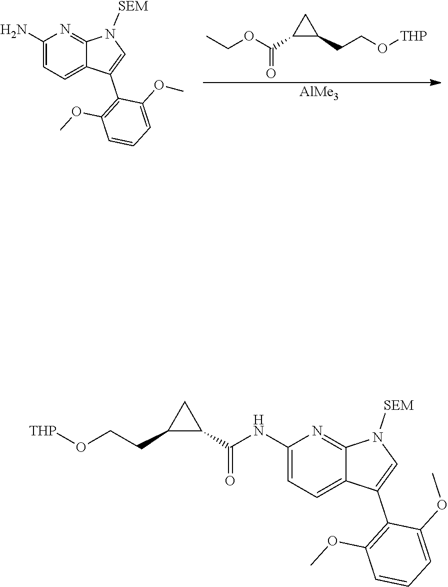

To a solution of 3-(2,6-dimethoxyphenyl)-1-((2-(trimethylsilyl)ethoxy)methyl)-1H-pyrrolo[2,3-b]pyridin-6-amine (900.0 mg, 2.52 mmol) in THE (10.0 mL) was added trans-ethyl-2-(2-((tetrahydro-2H-pyran-2-yl)oxy)ethyl)cyclopropane-1-carboxylate (818.7 mg, 3.38 mmol) and AlMe₃ (1.5 mL, 2 mol/L) at room temperature under N₂. The resulting mixture was stirred at 80° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (90/10, v/v) to afford trans-N-(3-(2,6-dimethoxyphenyl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide (1.3 g, 87%) as a brown solid. LCMS (ESI, m/z): [M+H]⁺=596.3.

### Step 2: Synthesis of Trans-N-(3-(2,6-dimethoxyphenyl)-1-((2-(trimethylsilyl)ethoxy)methyl)-1H-pyrrolo[2,3-b]pyridin-6-yl)-2-(2-hydroxyethyl)cyclopropane-1-carboxamide

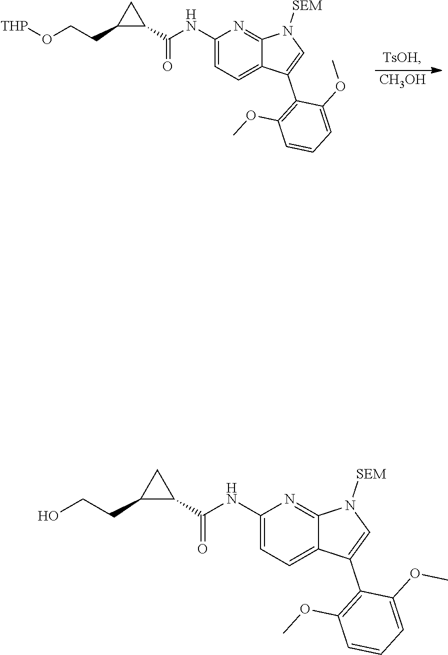

To a solution of trans-N-(3-(2,6-dimethoxyphenyl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide (1.1 g, 1.84 mmol) in CH₃OH (10.0 mL) was added TsOH (158.9 mg, 0.92 mmol) at room temperature. The resulting mixture was stirred at room temperature for 4 h. After the reaction was completed, the resulting mixture was concentrated under reduced pressure. the residue was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (88/12, v/v) to afford trans-N-(3-(2,6-dimethoxyphenyl)-1-((2-(trimethylsilyl)ethoxy)methyl)-1H-pyrrolo[2,3-b]pyridin-6-yl)-2-(2-hydroxyethyl)cyclopropane-1-carboxamide (930.0 mg, 88%) as a brown solid. LCMS (ESI, m/z): [M+H]⁺=512.3.

### Step 3: Synthesis of Trans-N-(3-(2,6-dimethoxyphenyl)-1-((2-(trimethylsilyl)ethoxy)methyl)-1H-pyrrolo[2,3-b]pyridin-6-yl)-2-(2-oxoethyl)cyclopropane-1-carboxamide

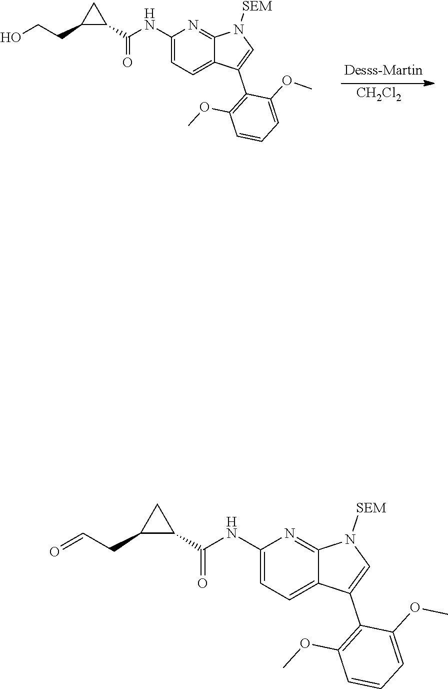

To a solution of trans-N-(3-(2,6-dimethoxyphenyl)-1-((2-(trimethylsilyl)ethoxy)methyl)-1H-pyrrolo[2,3-b]pyridin-6-yl)-2-(2-hydroxyethyl)cyclopropane-1-carboxamide (790.0 mg, 1.54 mmol) in CH₂Cl₂ (10.0 mL) was added Dess-Martin (982.2 mg, 2.32 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure to afford trans-N-(3-(2,6-dimethoxyphenyl)-1-((2-(trimethylsilyl)ethoxy)methyl)-1H-pyrrolo[2,3-b]pyridin-6-yl)-2-(2-oxoethyl)cyclopropane-1-carboxamide (790.0 mg, crude) as a brown solid. LCMS (ESI, m/z): [M+H]⁺=510.2.

### Step 4: Synthesis of Trans-N-(3-(2,6-dimethoxyphenyl)-1-((2-(trimethylsilyl)ethoxy)methyl)-1H-pyrrolo[2,3-b]pyridin-6-yl)-2-(2-(dimethylamino)ethyl)cyclopropane-1-carboxamide

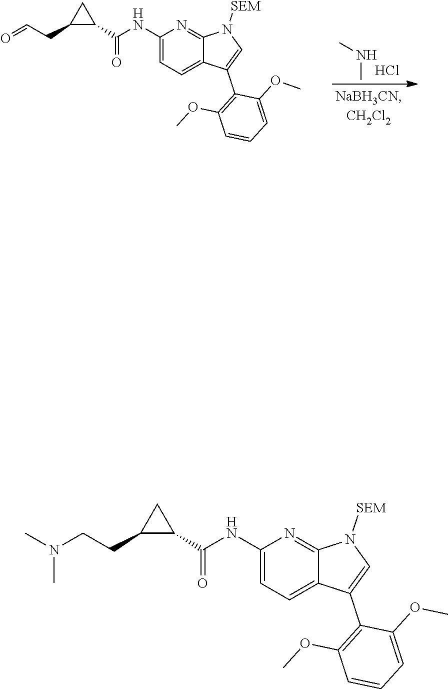

To a solution of trans-N-(3-(2,6-dimethoxyphenyl)-1-((2-(trimethylsilyl)ethoxy)methyl)-1H-pyrrolo[2,3-b]pyridin-6-yl)-2-(2-oxoethyl)cyclopropane-1-carboxamide (790.0 mg, crude) in CH₂Cl₂ (10.0 mL) was added dimethylamine hydrochloride (376.2 mg, 4.61 mmol) and NaBH₃CN (290.0 mg, 4.61 mmol) at room temperature under N₂. The resulting mixture was stirred at room temperature for 4 h. After the reaction was completed, the resulting mixture was quenched with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (90/10, v/v) to afford trans-N-(3-(2,6-dimethoxyphenyl)-1-((2-(trimethylsilyl)ethoxy)methyl)-1H-pyrrolo[2,3-b]pyridin-6-yl)-2-(2-(dimethylamino)ethyl)cyclopropane-1-carboxamide (750.0 mg, 81%) as a brown solid. LCMS (ESI, m/z): [M+H]⁺=539.3.

### Step 5: Synthesis of Trans-N-(3-(2,6-dimethoxyphenyl)-1H-pyrrolo[2,3-b]pyridin-6-yl)-2-(2-(dimethylamino)ethyl)cyclopropane-1-carboxamide

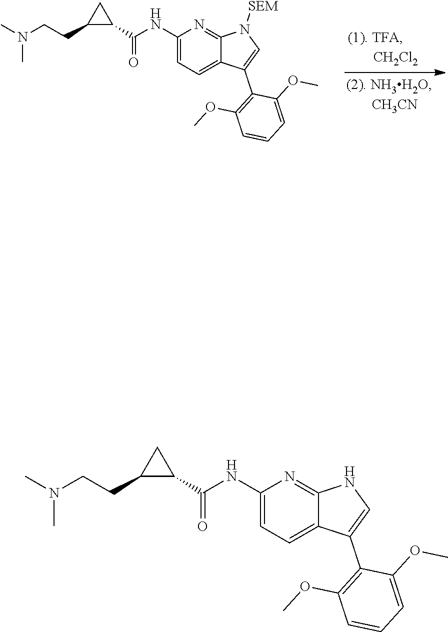

To a solution of trans-N-(3-(2,6-dimethoxyphenyl)-1-((2-(trimethylsilyl)ethoxy)methyl)-1H-pyrrolo[2,3-b]pyridin-6-yl)-2-(2-(dimethylamino)ethyl)cyclopropane-1-carboxamide (750.0 mg, 1.26 mmol) in CH₂Cl₂ (4.0 mL) was added TFA (4.0 mL) at room temperature. The resulting mixture was stirred at room temperature for 2 h. The resulting mixture was concentrated under reduced pressure. To the residue in CH₃CN (4.0 mL) was added NH₃·H₂O (4.0 mL) at room temperature. The resulting mixture was stirred at room temperature for 8 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford trans-N-(3-(2,6-dimethoxyphenyl)-1H-pyrrolo[2,3-b]pyridin-6-yl)-2-(2-(dimethylamino)ethyl)cyclopropane-1-carboxamide (200.0 mg, 27%) as a white solid. LCMS (ESI, m/z): [M+H]⁺=409.2.

### Step 6: Synthesis of (1S,2R)—N-(3-(2,6-dimethoxyphenyl)-1H-pyrrolo[2,3-b]pyridin-6-yl)-2-(2-(dimethylamino)ethyl)cyclopropane-1-carboxamide and (1R,2S)—N-(3-(2,6-dimethoxyphenyl)-1H-pyrrolo[2,3-b]pyridin-6-yl)-2-(2-(dimethylamino)ethyl)cyclopropane-1-carboxamide (Compound 224 and Compound 225)

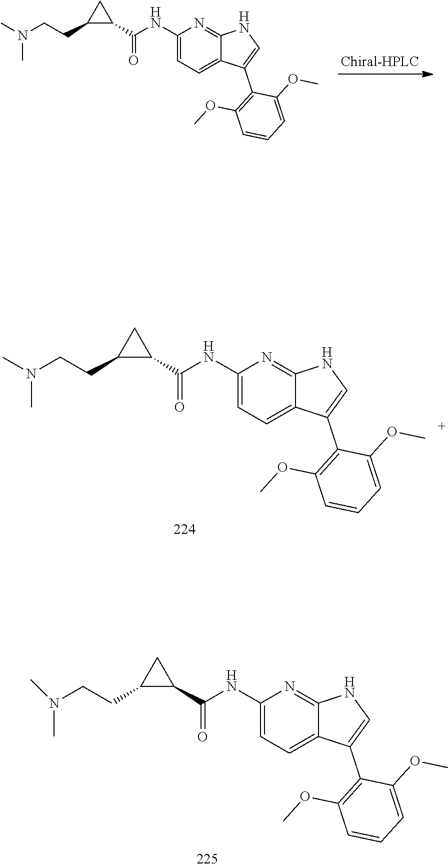

The product trans-N-(3-(2,6-dimethoxyphenyl)-1H-pyrrolo[2,3-b]pyridin-6-yl)-2-(2-(dimethylamino)ethyl)cyclopropane-1-carboxamide (200.0 mg, 0.48 mmol) was separated by Prep-Chiral-HPLC with the following conditions (Column: CHIRALPAK IH, 2×25 cm, 5 m; Mobile Phase A: Hex (0.5% 2M NH₃-MeOH)—HPLC, Mobile Phase B: MeOH: EtOH=1: 1—HPLC; Flow rate: 20 mL/min; Gradient: 20% B to 20% B in 19 min; Wave Length: 220/254 nm) to afford N-(3-(2,6-dimethoxyphenyl)-1H-pyrrolo[2,3-b]pyridin-6-yl)-2-(2-(dimethylamino)ethyl)cyclopropane-1-carboxamide Enantiomer 1 (23.0 mg, 23%) as a white solid and N-(3-(2,6-dimethoxyphenyl)-1H-pyrrolo[2,3-b]pyridin-6-yl)-2-(2-(dimethylamino)ethyl)cyclopropane-1-carboxamide Enantiomer 2 (20.6 mg, 20%) as a white solid. The absolute stereochemistry of Enantiomers 1 and 2 was not assigned. The two enantiomeric structures that could be obtained from chiral separation of the enantiomeric mixture as described above are shown as Compounds 224 and 225 in Table 1.

N-(3-(2,6-dimethoxyphenyl)-1H-pyrrolo[2,3-b]pyridin-6-yl)-2-(2-(dimethylamino)ethyl)cyclopropane-1-carboxamide Enantiomer 1: Retention Time 1 (min): 10.06; LCMS (ESI, m/z): [M+H]⁺=409.2. ¹H NMR (400 MHz, DMSO-d₆): δ 11.38 (s, 1H), 10.43 (s, 1H), 7.80 (d, J=8.8 Hz, 1H), 7.52 (d, J=8.4 Hz, 1H), 7.29-7.25 (m, 2H), 6.80-6.74 (m, 2H), 3.69 (s, 6H), 2.36-2.29 (m, 2H), 2.20-2.13 (m, 6H), 1.85-1.82 (m, 1H), 1.50-1.36 (m, 2H), 1.24-1.21 (m, 1H), 1.02-0.98 (m, 1H), 0.70-0.66 (m, 1H).

N-(3-(2,6-dimethoxyphenyl)-1H-pyrrolo[2,3-b]pyridin-6-yl)-2-(2-(dimethylamino)ethyl)cyclopropane-1-carboxamide Enantiomer 2: Retention Time 2 (min): 14.10; LCMS (ESI, m/z): [M+H]⁺=409.2. ¹H NMR (400 MHz, DMSO-d₆): δ 11.38 (s, 1H), 10.43 (s, 1H), 7.80 (d, J=8.4 Hz, 1H), 7.52 (d, J=8.8 Hz, 1H), 7.29-7.25 (m, 2H), 6.75 (d, J=8.4 Hz, 2H), 3.69 (s, 6H), 2.33-2.28 (m, 2H), 2.13 (s, 6H), 1.85-1.82 (m, 1H), 1.49-1.35 (m, 2H), 1.24-1.21 (m, 1H), 1.02-0.98 (m, 1H), 0.70-0.66 (m, 1H).

### Example S206: Synthesis of (R)-1-(3-(4,6-dimethoxypyrimidin-5-yl)-1H-pyrrolo[2,3-b]pyridin-6-yl)-3-(3-(dimethylamino)-2-fluoropropyl)urea and (S)-1-(3-(4,6-dimethoxypyrimidin-5-yl)-1H-pyrrolo[2,3-b]pyridin-6-yl)-3-(3-(dimethylamino)-2-fluoropropyl)urea (Compound 226 and Compound 227)

### Step 1: Synthesis of 1-[3-(4,6-dimethoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea

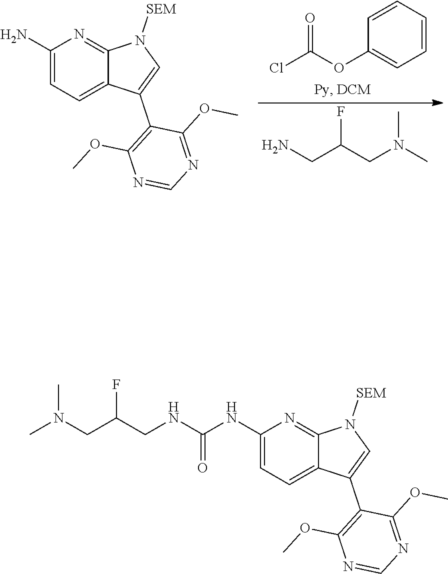

To a solution of 3-(4,6-dimethoxypyrimidin-5-yl)-1-{[2-(trimethylsilyl)ethoxy]methyl}pyrrolo[2,3-b]pyridin-6-amine (474.0 mg, 1.18 mmol) in DCM (10.0 mL) was added Pyridine (373.5 mg, 4.72 mmol) and phenyl chloroformate (369.7 mg, 2.36 mmol) at room temperature. The resulting mixture was stirred at room temperature for 3 h. The resulting mixture was concentrated under reduced pressure. To the above residue was added Pyridine (10.0 mL) and (3-amino-2-fluoropropyl)dimethylamine (1418.6 mg, 11.80 mmol) at room temperature. The resulting mixture was stirred at 60° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (20/1, v/v) to afford 1-[3-(4,6-dimethoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea (440.0 mg, 68%) as a yellow solid. LCMS (ESI, m/z): [M+H]⁺=548.3.

### Step 2: Synthesis of 1-(3-(4,6-dimethoxypyrimidin-5-yl)-1H-pyrrolo[2,3-b]pyridin-6-yl)-3-(3-(dimethylamino)-2-fluoropropyl)urea

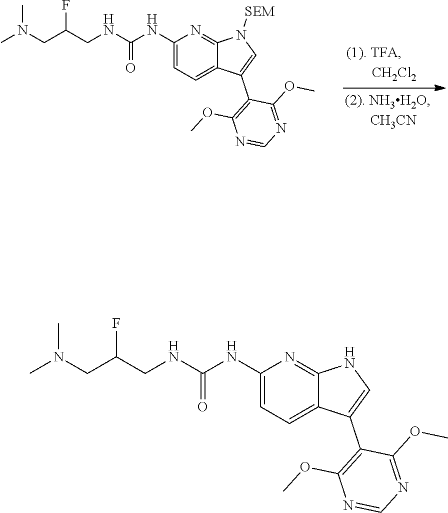

To a solution of 1-[3-(4,6-dimethoxypyrimidin-5-yl)-1-{[2-(trimethylsilyl)ethoxy]methyl}pyrrolo[2,3-b]pyridin-6-yl]-3-[3-(dimethylamino)-2-fluoropropyl]urea (410.0 mg, 0.75 mmol) in CH₂Cl₂ (8.0 mL) was added TFA (8.0 mL) at room temperature. The resulting mixture was stirred at room temperature for 3 h. The resulting mixture was concentrated under reduced pressure. To the above residue was added CH₃CN (8.0 mL) and NH₃·H₂O (8.0 mL) at room temperature. The resulting mixture was stirred at room temperature for another 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography eluted with methanol/H₂O (3/2, v/v) to afford 1-(3-(4,6-dimethoxypyrimidin-5-yl)-1H-pyrrolo[2,3-b]pyridin-6-yl)-3-(3-(dimethylamino)-2-fluoropropyl)urea (95.0 mg, 30%) as a yellow solid. LCMS (ESI, m/z): [M+H]⁺=418.2.

### Step 3: Synthesis of (R)-1-(3-(4,6-dimethoxypyrimidin-5-yl)-1H-pyrrolo[2,3-b]pyridin-6-yl)-3-(3-(dimethylamino)-2-fluoropropyl)urea and (S)-1-(3-(4,6-dimethoxypyrimidin-5-yl)-1H-pyrrolo[2,3-b]pyridin-6-yl)-3-(3-(dimethylamino)-2-fluoropropyl)urea (Compound 226 and Compound 227)

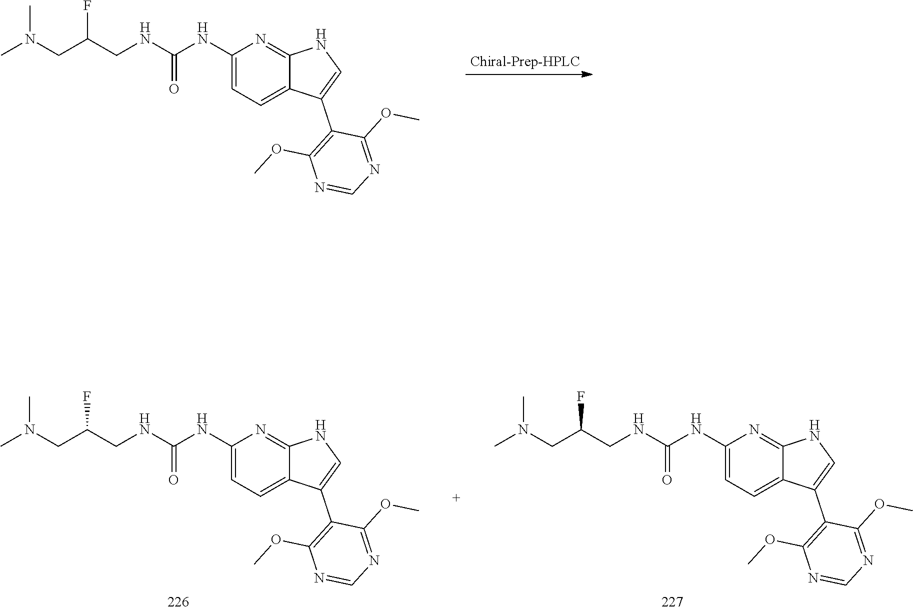

The racemic 1-(3-(4,6-dimethoxypyrimidin-5-yl)-1H-pyrrolo[2,3-b]pyridin-6-yl)-3-(3-(dimethylamino)-2-fluoropropyl)urea (95.0 mg, 0.23 mmol) was separated by Prep-Chiral-HPLC with the following conditions (Column: CHIRAL ART Amylose-SA, 2×25 cm, m; Mobile Phase A: Hex (0.5% 2M NH₃-MeOH)—HPLC, Mobile Phase B: EtOH—HPLC; Flow rate: 20 mL/min; Gradient: 30% B to 30% B in 22 min; Wave Length: 220/254 nm) to afford 1-(3-(4,6-dimethoxypyrimidin-5-yl)-1H-pyrrolo[2,3-b]pyridin-6-yl)-3-(3-(dimethylamino)-2-fluoropropyl)urea Enantiomer 1 (18.9 mg, 20%) as a white solid and 1-(3-(4,6-dimethoxypyrimidin-5-yl)-1H-pyrrolo[2,3-b]pyridin-6-yl)-3-(3-(dimethylamino)-2-fluoropropyl)urea Enantiomer 2 (11.5 mg, 18%) as a white solid. The absolute stereochemistry of Enantiomers 1 and 2 was not assigned. The two enantiomeric structures that could be obtained from chiral separation of the enantiomeric mixture as described above are shown as Compounds 226 and 227 in Table 1.

1-(3-(4,6-dimethoxypyrimidin-5-yl)-1H-pyrrolo[2,3-b]pyridin-6-yl)-3-(3-(dimethylamino)-2-fluoropropyl)urea Enantiomer 1: Retention Time 1=10.16 min; LCMS (ESI, m/z): [M+H]⁺=418.3. ¹H NMR (400 MHz, DMSO-d₆): δ 11.61 (s, 1H), 9.27 (s, 1H), 8.61 (s, 1H), 8.46 (s, 1H), 7.65 (s, 1H), 7.37 (s, 1H), 7.05 (s, 1H), 4.81-4.69 (m, 1H), 3.91 (s, 6H), 3.74-3.55 (m, 1H), 2.21 (s, 6H).

1-(3-(4,6-dimethoxypyrimidin-5-yl)-1H-pyrrolo[2,3-b]pyridin-6-yl)-3-(3-(dimethylamino)-2-fluoropropyl)urea Enantiomer 2: Retention Time 2=17.388 min; LCMS (ESI, m/z): [M+H]⁺=418.3. ¹H NMR (400 MHz, DMSO-d₆): δ 11.62 (s, 1H), 9.27 (s, 1H), 8.61 (s, 1H), 8.46 (s, 1H), 7.65 (d, J=7.6 Hz, 1H), 7.37 (s, 1H), 7.05 (d, J=7.6 Hz, 1H), 4.81-4.69 (m, 1H), 3.91 (s, 6H), 3.60-3.54 (m, 1H), 2.21 (s, 6H).

### Example S207: Synthesis of trans-N-(3-(2,6-dimethoxyphenyl)-1H-pyrrolo[2,3-b]pyridin-6-yl)-3(R)-fluoro-2-((4-methylpiperazin-1-yl)methyl)cyclopropane-1-carboxamide (Compound 228)

### Step 1: Synthesis of (E)-4-(benzyloxy)but-2-en-1-ol

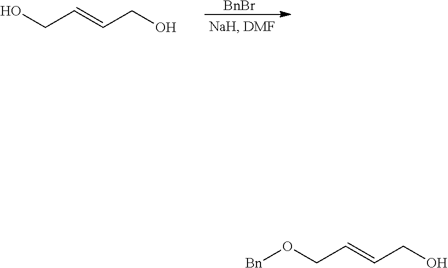 [SMILES: "BnBr" ; OC/C=C/CO ; OC/C=C/COCc1ccccc1]

To a solution of NaH (16.3 g, 60% purity) in DMF (250.0 mL) was added dropwise a solution of (E)-but-2-ene-1,4-diol (50.0 g, 567.50 mmol) in DMF (250.0 mL) at 0° C. under N₂. The resulting mixture was stirred at 0° C. for 1 h under N₂. Then a solution of BnBr (97.1 g, 567.50 mmol) in DMF (100.0 mL) was added dropwise to the mixture at 0° C. under N₂. The resulting mixture was stirred at 0° C. for additional 1 h under N₂. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (75/25, v/v) to afford (E)-4-(benzyloxy)but-2-en-1-ol (70.0 g, 69%) as a colorless oil. LCMS (ESI, m/z): [M+H]⁺=179.1.

### Step 2: Synthesis of (E)-((4-(benzyloxy)but-2-en-1-yl)oxy)triisopropylsilane

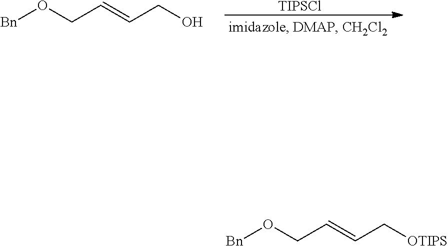

To a solution of (E)-4-(benzyloxy)but-2-en-1-ol (50.0 g, 280.54 mmol) in CH₂Cl₂ (500.0 mL) was added imidazole (42.0 g, 617.17 mmol), DMAP (6.9 g, 56.11 mmol) and TIPSCl (108.2 g, 561.07 mmol) at room temperature. The resulting mixture was stirred at room temperature for 4 h. After the reaction was completed, the resulting mixture was concentrated under reduced pressure. The residue was purified by flash column chromatography with petroleum ether/ethyl acetate (96/4, v/v) to afford (E)-((4-(benzyloxy)but-2-en-1-yl)oxy)triisopropylsilane (90.0 g, 95%) as a colorless oil. LCMS (ESI, m/z): [M+H]⁺=335.2.

### Step 3: Synthesis of (((1S/R,3S/R)-3-((benzyloxy)methyl)-2-bromo-2-fluorocyclopropyl)methoxy)triisopropylsilane

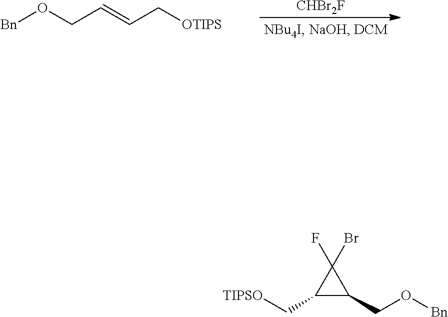

To a solution of (E)-((4-(benzyloxy)but-2-en-1-yl)oxy)triisopropylsilane (59.0 g, 132.88 mmol) in DCM (300.0 mL) and NaOH (300.0 mL, 50%) was added NBu₄I (4.90 g, 13.29 mmol) and CHBr₂F (50.3 g, 199.32 mmol) at room temperature. The resulting mixture was stirred at room temperature for 12 h. After the reaction was completed, the resulting mixture was quenched with H₂O at 0° C. and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford (((1S/R,3S/R)-3-((benzyloxy)methyl)-2-bromo-2-fluorocyclopropyl)methoxy)triisopropylsilane (50.0 g, crude) as a colorless oil. LCMS (ESI, m/z): [M+H]⁺=445.1.

### Step 4: Synthesis of (trans-(2-((benzyloxy)methyl)-3-fluorocyclopropyl)methoxy)triisopropylsilane

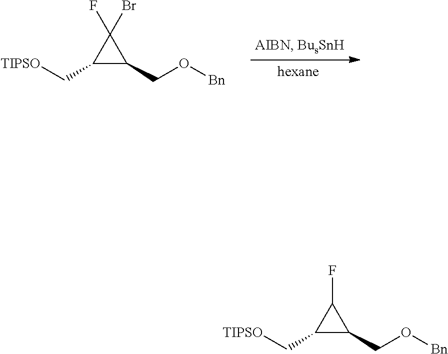

To a solution of (((1S/R,3S/R)-3-((benzyloxy)methyl)-2-bromo-2-fluorocyclopropyl)methoxy)triisopropylsilane (50.0 g, 112.24 mmol) in hexane (500.0 mL) was added Bu₃SnH (35.9 g, 123.4 mmol) and AIBN (1.8 g, 11.22 mmol) at room temperature. The resulting mixture was stirred at 90° C. for 4 h. After the reaction was completed, the resulting mixture was concentrated under reduced pressure. The residue was purified by flash column chromatography with petroleum ether/ethyl acetate (88/12, v/v) to afford (trans-(2-((benzyloxy)methyl)-3-fluorocyclopropyl)methoxy)triisopropylsilane (50.0 g, crude) as a colorless oil. LCMS (ESI, m/z): [M+H]⁺=367.2.

### Step 5: Synthesis of trans-(2-((benzyloxy)methyl)-3(S)-fluorocyclopropyl)methanol

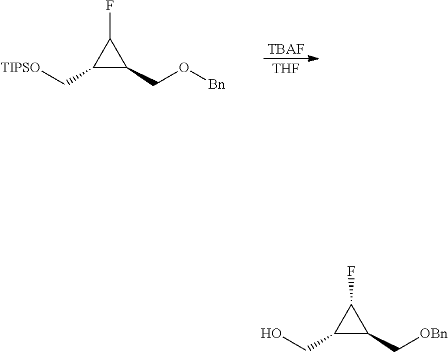

To a solution of (trans-(2-((benzyloxy)methyl)-3-fluorocyclopropyl)methoxy)triisopropylsilane (50.0 g, 136.39 mmol) in THE (500.0 mL) was added TBAF (42.8 g, 163.67 mmol) at 0° C. under N₂. The resulting mixture was stirred at 0° C. for 1 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (40/60, v/v) to afford trans-(2-((benzyloxy)methyl)-3(S)-fluorocyclopropyl)methanol (1.5 g, 10%) as a colorless oil. LCMS (ESI, m/z): [M+H]⁺=211.1.

### Step 6: Synthesis of trans-(2-((benzyloxy)methyl)-3(R)-fluorocyclopropyl)-1-carbaldehyde

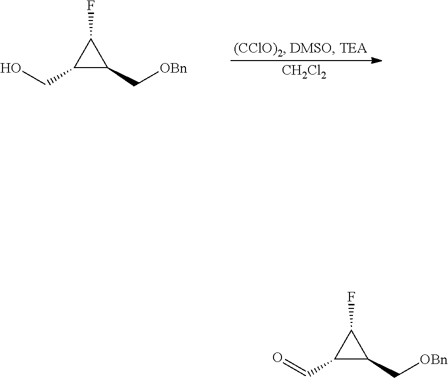 [SMILES: OC[C@@H]1[C@H](F)[C@H]1COCc1ccccc1 ; "(CClO)2, DMSO, TEA" ; "CH2Cl2" ; O=C[C@@H]1[C@H](F)[C@H]1COCc1ccccc1]

To a solution of (COCl)₂ (1.8 g, 14.27 mmol) in CH₂Cl₂ (10.0 mL) was added dropwise a solution of DMSO (4.5 g, 57.08 mmol) in CH₂Cl₂ (10.0 mL) at −78° C. under N₂. The resulting mixture was stirred at −78° C. for 0.5 h under N₂. Then a solution of trans-(2-((benzyloxy)methyl)-3(S)-fluorocyclopropyl)methanol (1.5 g, 7.13 mmol) in CH₂Cl₂ (10.0 mL) was added dropwise to the mixture at −78° C. under N₂. The resulting mixture was stirred at −78° C. for additional 1 h. After the reaction was completed, TEA (5.8 g, 57.08 mmol) was added dropwise to the mixture at −78° C. under N₂. The resulting mixture was stirred at −78° C. for another 0.5 h under N₂. The resulting mixture was quenched with aqueous NH₄Cl and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (90/10, v/v) to afford trans-(2-((benzyloxy)methyl)-3(R)-fluorocyclopropyl)-1-carbaldehyde (410.0 mg, 25%) as a colorless oil. LCMS (ESI, m/z): [M+H]⁺=209.1.

### Step 7: Synthesis of trans-(2-((benzyloxy)methyl)-3(R)-fluorocyclopropyl)-1-carboxylic acid

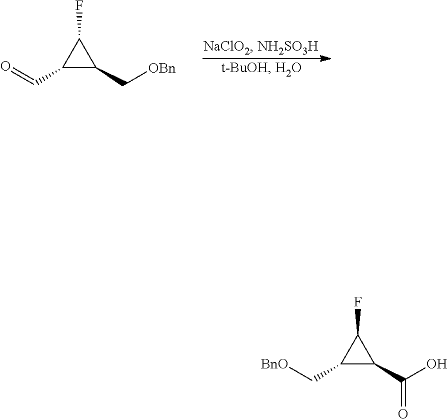

To a mixture of trans-(2-((benzyloxy)methyl)-3(R)-fluorocyclopropyl)-1-carbaldehyde (360.0 mg, 1.73 mmol) and NH₂SO₃H (1.7 g, 17.29 mmol) in t-BuOH/H₂O (1.0/8.0 mL) was added NaClO₂ (1.6 g, 17.29 mmol) at 0° C. under N₂. The resulting mixture was stirred at room temperature for 1.5 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (50/50, v/v) to afford trans-(2-((benzyloxy)methyl)-3(R)-fluorocyclopropyl)-1-carboxylic acid (80.0 mg, 20%) as a colorless oil. LCMS (ESI, m/z): [M+H]⁺=225.1.

### Step 8: Synthesis of trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(R)-fluorocyclopropane-1-carboxamide

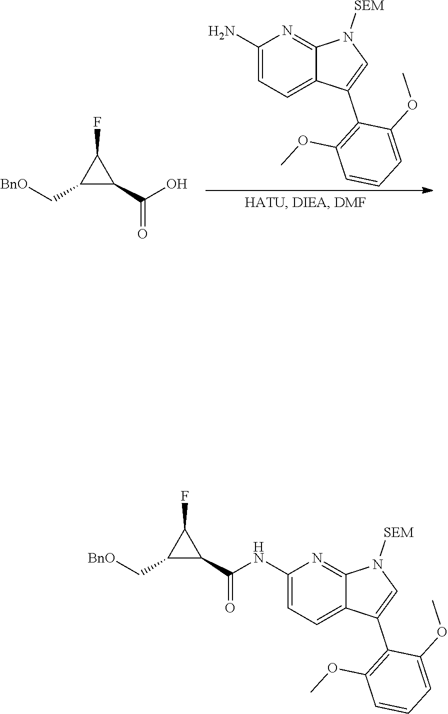 [SMILES: O=C(O)[C@@H]1[C@H](F)[C@H]1COCc1ccccc1 ; COc1cccc(OC)c1-c1cn(COCC[Si](C)(C)C)c2nc(N)ccc12 ; COc1cccc(OC)c1-c1cn(COCC[Si](C)(C)C)c2nc(NC(=O)[C@@H]3[C@H](F)[C@H]3COCc3ccccc3)ccc12]

To a solution of 3-(2,6-dimethoxyphenyl)-1-((2-(trimethylsilyl)ethoxy)methyl)-1H-pyrrolo[2,3-b]pyridin-6-amine (231.7 mg, 0.58 mmol) in DMF (6.0 mL) were added trans-(2-((benzyloxy)methyl)-3(R)-fluorocyclopropyl)-1-carboxylic acid (130.0 mg, 0.58 mmol), DIEA (224.8 mg, 1.74 mmol) and HATU (264.5 mg, 0.70 mmol) at 0° C. under N₂. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (90/10, v/v) to afford trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(R)-fluorocyclopropane-1-carboxamide (270.0 mg, 77%) as a white solid. LCMS (ESI, m/z): [M+H]⁺=606.3.

### Step 9: Synthesis of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-(hydroxymethyl)cyclopropane-1-carboxamide

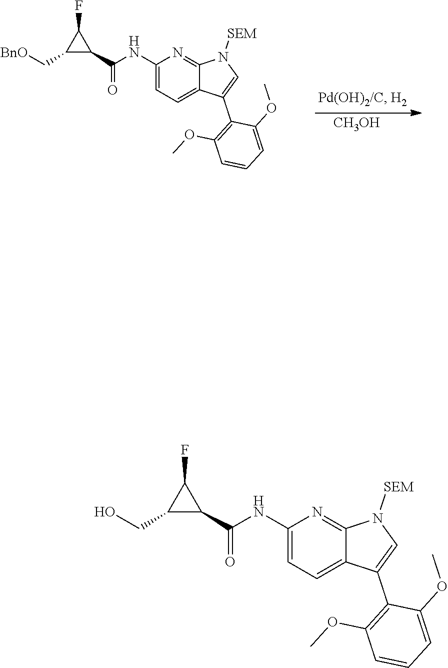 [SMILES: COc1cccc(OC)c1-c1cn(COCC[Si](C)(C)C)c2nc(NC(=O)[C@@H]3[C@H](F)[C@H]3COCc3ccccc3)ccc12 ; "CH3OH" ; COc1cccc(OC)c1-c1cn(COCC[Si](C)(C)C)c2nc(NC(=O)[C@@H]3[C@H](F)[C@H]3CO)ccc12]

To a solution of trans-2-((benzyloxy)methyl)-N-(3-(2,6-dimethoxyphenyl)-1-((2-(trimethylsilyl)ethoxy)methyl)-1H-pyrrolo[2,3-b]pyridin-6-yl)-3(R)-fluorocyclopropane-1-carboxamide (230.0 mg, 0.38 mmol) in CH₃OH (5.0 mL) was Pd(OH)₂/C (106.1 mg, dry) at room temperature under N₂. The resulting mixture was stirred at room temperature for 4 h under H₂. After the reaction was completed, the resulting mixture was filtered. The filtrate was concentrated under reduced pressure. The residue was purified by reverse flash column chromatography with CH₃CN/H₂O (28/72, v/v) to afford trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-(hydroxymethyl)cyclopropane-1-carboxamide (200.0 mg, 97%) as a white solid. LCMS (ESI, m/z): [M+H]⁺=516.2.

### Step 10: Synthesis of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-formylcyclopropane-1-carboxamide

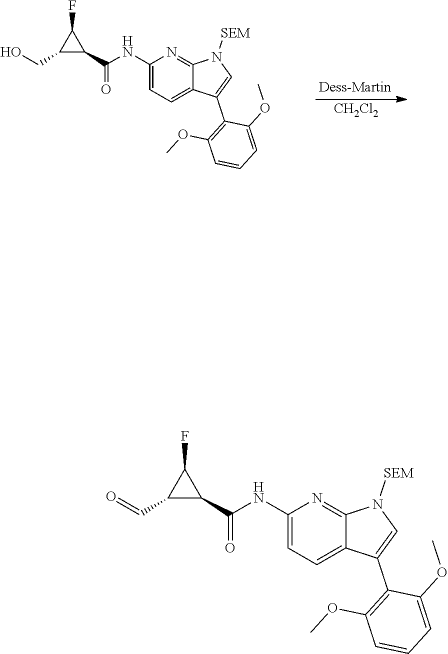 [SMILES: COc1cccc(OC)c1-c1cn(COCC[Si](C)(C)C)c2nc(NC(=O)[C@@H]3[C@H](F)[C@H]3CO)ccc12 ; "CH2Cl2" ; COc1cccc(OC)c1-c1cn(COCC[Si](C)(C)C)c2nc(NC(=O)[C@@H]3[C@H](F)[C@H]3C=O)ccc12]

To a solution of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-(hydroxymethyl)cyclopropane-1-carboxamide (200.0 mg, 0.39 mmol) in CH₂Cl₂ (10.0 mL) was added Dess-Martin (246.8 mg, 0.58 mmol) at room temperature. The resulting mixture was stirred at room temperature for 2 h. After the reaction was completed, the resulting mixture was diluted with H₂O and extracted with CH₂Cl₂. The combined organic layer was washed with aq. NaHCO₃, brine, dried over anhydrous sodium sulfate and filtered. The filtrate was concentrated under reduced pressure to afford trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-formylcyclopropane-1-carboxamide (200.0 mg, crude) as a brown solid. LCMS (ESI, m/z): [M+H]⁺=514.2.

### Step 11: Synthesis of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-((4-methylpiperazin-1-yl)methyl)cyclopropane-1-carboxamide

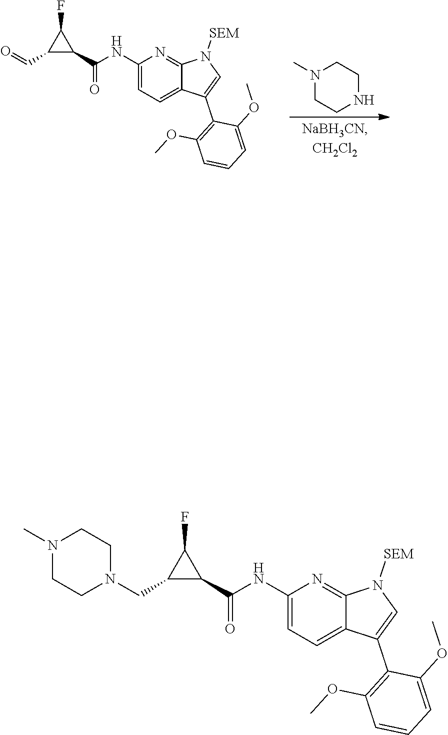

To a solution of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-formylcyclopropane-1-carboxamide (200.0 mg, crude) in CH₂Cl₂ (10.0 mL) was added 1-methylpiperazine (117.0 mg, 1.17 mmol) and NaBH₃CN (110.1 mg, 1.17 mmol) at room temperature. The resulting mixture was stirred at room temperature for 2 h. After the reaction was completed, the resulting mixture was quenched with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/CH₃OH (20/1, v/v) to afford trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-((4-methylpiperazin-1-yl)methyl)cyclopropane-1-carboxamide (75.0 mg, 34%) as a brown solid. LCMS (ESI, m/z): [M+H]⁺=598.3.

### Step 12: Synthesis of trans-N-(3-(2,6-dimethoxyphenyl)-1H-pyrrolo[2,3-b]pyridin-6-yl)-3(R)-fluoro-2-((4-methylpiperazin-1-yl)methyl)cyclopropane-1-carboxamide (Compound 228)

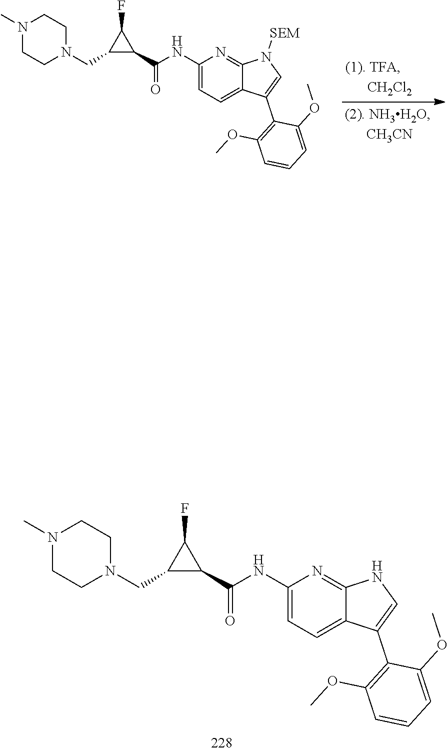 [SMILES: COc1cccc(OC)c1-c1cn(COCC[Si](C)(C)C)c2nc(NC(=O)[C@@H]3[C@H](F)[C@H]3CN3CCN(C)CC3)ccc12 ; COc1cccc(OC)c1-c1c[nH]c2nc(NC(=O)[C@@H]3[C@H](F)[C@H]3CN3CCN(C)CC3)ccc12]

To a solution of trans-N-(3-(2,6-dimethoxyphenyl)-1-((2-(trimethylsilyl)ethoxy)methyl)-1H-pyrrolo[2,3-b]pyridin-6-yl)-3(R)-fluoro-2-((4-methylpiperazin-1-yl)methyl)cyclopropane-1-carboxamide (70.0 mg, 0.27 mmol) in CH₂Cl₂ (4.0 mL) was added TFA (4.0 mL) at room temperature. The resulting mixture was stirred at room temperature for 2 h. The resulting mixture was concentrated under reduced pressure. To the residue in CH₃CN (4.0 mL) was added NH₃·H₂O (4.0 mL) at room temperature. The resulting mixture was stirred at room temperature for additional 8 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 27% B to 37% B in 8 min; Wave Length: 254 nm) to afford trans-N-(3-(2,6-dimethoxyphenyl)-1H-pyrrolo[2,3-b]pyridin-6-yl)-3(R)-fluoro-2-((4-methylpiperazin-1-yl)methyl)cyclopropane-1-carboxamide (Compound 228) (3.6 mg, 7%) as a white solid. LCMS (ESI, m/z): [M+H]⁺=468.2. ¹H NMR (400 MHz, DMSO-d₆): δ 11.37 (s, 1H), 10.88 (s, 1H), 7.78 (d, J=8.0 Hz, 1H), 7.54 (d, J=8.4 Hz, 1H), 7.30-7.26 (m, 2H), 6.76 (d, J=8.4 Hz, 2H), 5.07-4.87 (m, 1H), 3.70 (s, 6H), 2.90-2.79 (m, 2H), 2.72-2.55 (m, 1H), 2.45-2.23 (m, 5H), 2.21-2.10 (m, 4H), 1.56-1.39 (m, 1H).

### Example S208: Synthesis of (1S,2S)—N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide and (1R,2R)—N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide (Compound 229 and Compound 230)

### Step 1: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((4-ethylpiperazin-1-yl)methyl)cyclopropane-1-carboxamide

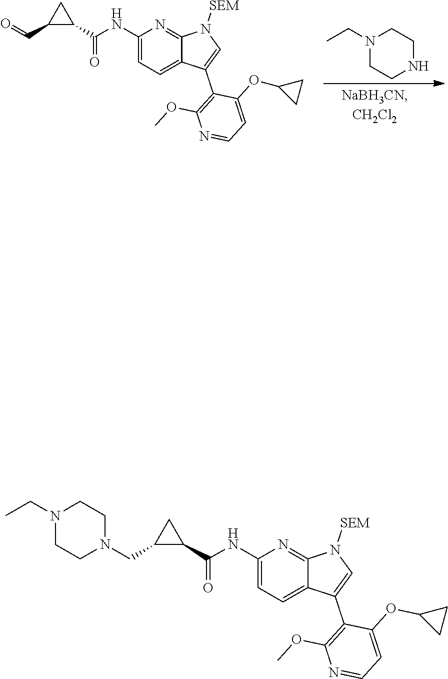

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-formylcyclopropane-1-carboxamide (250.0 mg, 0.47 mmol) in DCM (4.0 mL) was added NaBH₃CN (90.1 mg, 1.43 mmol) and 1-ethylpiperazine (163.8 mg, 1.43 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (10/1, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((4-ethylpiperazin-1-yl)methyl)cyclopropane-1-carboxamide (75.0 mg, 25%) as a yellow solid. LCMS (ESI, m/z): [M+H]⁺=621.4

### Step 2: Synthesis of Trans-N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide

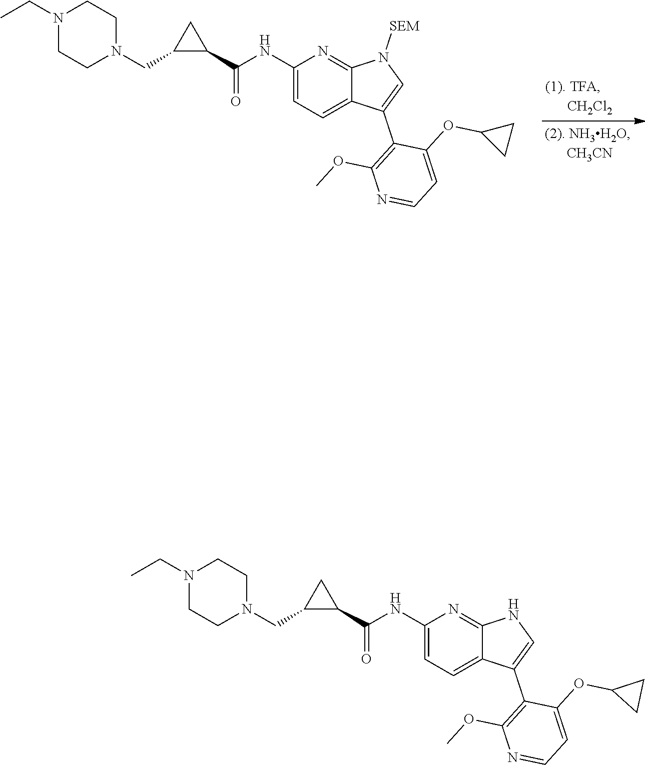 [SMILES: CCN1CCN(C[C@@H]2C[C@H]2C(=O)Nc2ccc3c(-c4c(OC5CC5)ccnc4OC)cn(COCC[Si](C)(C)C)c3n2)CC1 ; CCN1CCN(C[C@@H]2C[C@H]2C(=O)Nc2ccc3c(-c4c(OC5CC5)ccnc4OC)c[nH]c3n2)CC1]

To a solution of trans-N-[3-(4-cyclopropoxy-2-methoxypyridin-3-yl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide (150.0 mg, 0.09 mmol) in DCM (2.0 mL) was added TFA (1.0 mL) at room temperature. The resulting mixture was stirred at room temperature for 2 h. The resulting mixture was concentrated under reduced pressure. To the above residue was added NH₃·H₂O (1.0 mL) and ACN (1.0 mL) at room temperature. The resulting mixture was stirred at room temperature for additional 16 h. After the reaction was completed, the resulting mixture was concentrated under reduced pressure. The residue was purified by reverse phase flash chromatography with ACN/H₂O (2/1, v/v) to afford trans-N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide (60.0 mg, 51%) as a yellow solid. LCMS (ESI, m/z): [M+H]⁺=491.3

### Step 3: Synthesis of (1S,2S)—N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide and (1R,2R)—N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide (Compound 229 and Compound 230)

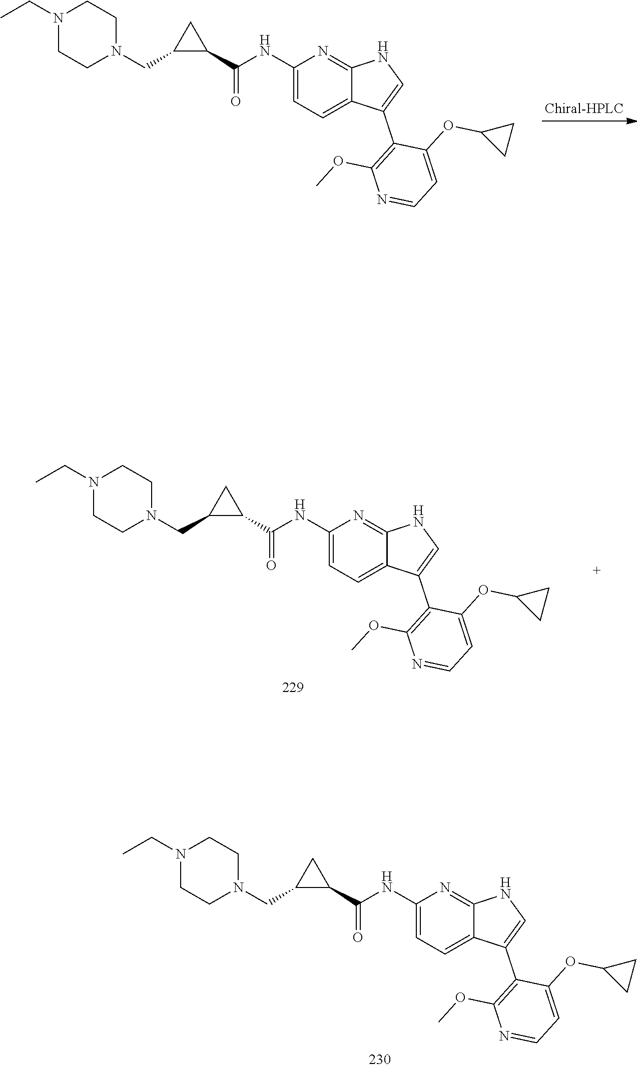 [SMILES: CCN1CCN(C[C@@H]2C[C@H]2C(=O)Nc2ccc3c(-c4c(OC5CC5)ccnc4OC)c[nH]c3n2)CC1 ; CCN1CCN(C[C@H]2C[C@@H]2C(=O)Nc2ccc3c(-c4c(OC5CC5)ccnc4OC)c[nH]c3n2)CC1 ; CCN1CCN(C[C@@H]2C[C@H]2C(=O)Nc2ccc3c(-c4c(OC5CC5)ccnc4OC)c[nH]c3n2)CC1]

The trans-N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide (60.0 mg) was separated by Prep-chiral HPLC with the following conditions (Column: CHIRAL ART Cellulose-SC, 2×25 cm, 5 m; Mobile Phase A: Hex (0.5% 2M NH₃-MeOH)—HPLC, Mobile Phase B: EtOH—HPLC; Flow rate: 20 mL/min; Gradient: 30% B to 30% B in 11 min; Wave Length: 220/254 nm) to afford N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide Enantiomer 1 (11.8 mg, 39%) and N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide Enantiomer 2 (17.4 mg, 56%) as a white solid. The absolute stereochemistry of Enantiomers 1 and 2 was not assigned. The two enantiomeric structures that could be obtained from chiral separation of the enantiomeric mixture as described above are shown as Compounds 229 and 230 in Table 1.

N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide Enantiomer 1: Retention Time 1=7.61 min; LCMS (ESI, m/z): [M+H]⁺=491.2. ¹H NMR (400 MHz, DMSO-d₆): δ 11.50 (s, 1H), 10.53 (s, 1H), 8.07 (d, J=6.0 Hz, 1H), 7.83 (d, J=8.4 Hz, 1H), 7.56 (d, J=8.8 Hz, 1H), 7.34 (d, J=2.0 Hz, 1H), 7.14 (d, J=2.0 Hz, 1H), 3.99-3.91 (m, 1H), 3.81 (s, 3H), 2.51-2.08 (m, 10H), 1.96-1.90 (m, 1H), 1.34-1.24 (m, 1H), 1.15-0.96 (m, 4H), 0.81-0.76 (m, 2H), 0.72-0.59 (m, 3H).

N-[3-(4-cyclopropoxy-2-methoxypyridin-3-yl)-1H-pyrrolo[2,3-b]pyridin-6-yl]-2-[(4-ethylpiperazin-1-yl)methyl]cyclopropane-1-carboxamide Enantiomer 2 Retention Time 2=9.31 min; LCMS (ESI, m/z): [M+H]⁺=491.3. ¹H NMR (400 MHz, DMSO-d₆): δ 11.50 (s, 1H), 10.53 (s, 1H), 8.07 (d, J=5.6 Hz, 1H), 7.83 (d, J=8.4 Hz, 1H), 7.56 (d, J=8.4 Hz, 1H), 7.35 (d, J=2.0 Hz, 1H), 7.14 (d, J=5.6 Hz, 1H), 3.94-3.91 (m, 1H), 3.82 (s, 3H), 2.68-2.21 (m, 10H), 1.91-1.88 (m, 1H), 1.34-1.30 (m, 1H), 1.06-0.96 (m, 4H), 0.81-0.76 (m, 2H), 0.72-0.64 (m, 3H).

### Example S209: Synthesis of (1S,2S)—N-[3-(5-chloro-2,4-dimethoxypyridin-3-yl)-1H-pyrrolo[2,3-b]pyridin-6-yl]-2-fluorocyclopropane-1-carboxamide (Compound 231)

### Step 1: Synthesis of 5-chloro-2,4-dimethoxypyridine

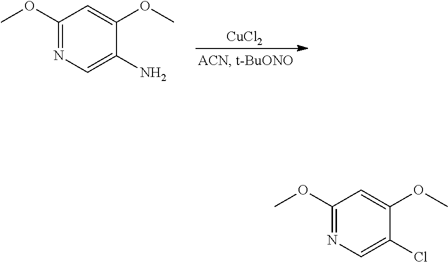

To a solution of 4,6-dimethoxypyridin-3-amine (2.0 g, 12.97 mmol) in MeCN (20.0 mL) was added t-BuONO (2.0 g, 19.46 mmol), CuCl₂ (0.8 g, 6.48 mmol) at room temperature. The resulting mixture was stirred at 80° C. for 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (9/1, v/v) to afford 5-chloro-2,4-dimethoxypyridine (900.0 mg, 39%) as a white solid. LCMS (ESI, m/z): [M+H]⁺=174.0

### Step 2: Synthesis of 3-bromo-5-chloro-2,4-dimethoxypyridine

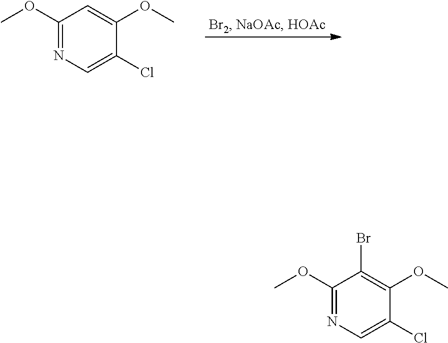

To a solution of 5-chloro-2,4-dimethoxypyridine (500.0 mg, 2.88 mmol) in AcOH (8.0 mL) was added NaOAc (236.2 mg, 2.88 mmol) and Br₂ (690.4 mg, 4.32 mmol) at room temperature. The resulting mixture was stirred at 80° C. for 16 h. After the reaction was completed, the pH value of the mixture was adjusted to 7 with aq·NaHCO₃. The mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford 3-bromo-5-chloro-2,4-dimethoxypyridine (450.0 mg, 61%) as a white solid. LCMS (ESI, m/z): [M+H]⁺=251.9

### Step 3: Synthesis of 5-chloro-3-(6-chloro-1-{[2-(trimethylsilyl)ethoxy]methyl}pyrrolo[2,3-b]pyridin-3-yl)-2,4-dimethoxypyridine

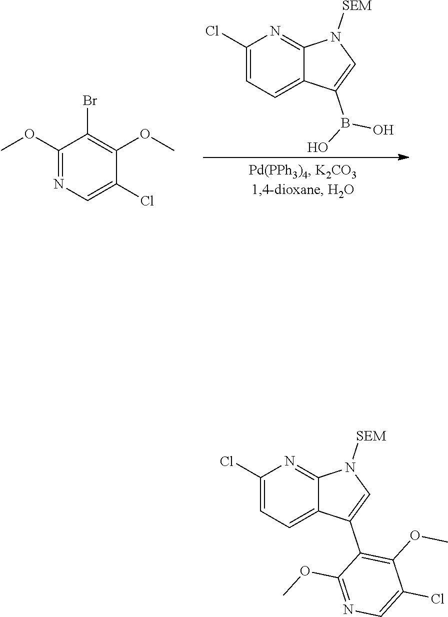

To a solution of 3-bromo-5-chloro-2,4-dimethoxypyridine (400.0 mg, 1.67 mmol) in 1,4-dioxane/H₂O (10.0/2.0 mL) was added Pd(PPh₃)₄ (387.6 mg, 0.33 mmol), 6-chloro-1-{[2-(trimethylsilyl)ethoxy]methyl}pyrrolo[2,3-b]pyridin-3-ylboronic acid (657.5 mg, 2.01 mmol) and K₂CO₃ (695.4 mg, 5.03 mmol) at room temperature under N₂. The resulting mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (6/1, v/v) to afford 5-chloro-3-(6-chloro-1-{[2-(trimethylsilyl)ethoxy]methyl}pyrrolo[2,3-b]pyridin-3-yl)-2,4-dimethoxypyridine (250.0 mg, 32%) as a yellow solid. LCMS (ESI, m/z): [M+H]⁺=454.1.

### Step 4: Synthesis of (1S,2S)—N-[3-(5-chloro-2,4-dimethoxypyridin-3-yl)-1-{[2-(trimethylsilyl)ethoxy]methyl}pyrrolo[2,3-b]pyridin-6-yl]-2-fluorocyclopropane-1-carboxamide

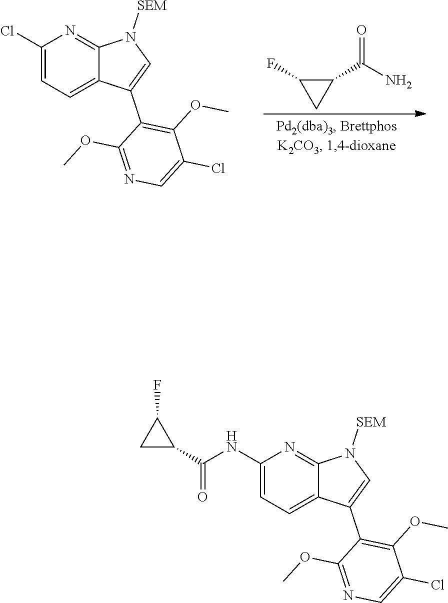 [SMILES: COc1ncc(Cl)c(OC)c1-c1cn(COCC[Si](C)(C)C)c2nc(Cl)ccc12 ; NC(=O)[C@@H]1C[C@@H]1F ; COc1ncc(Cl)c(OC)c1-c1cn(COCC[Si](C)(C)C)c2nc(NC(=O)[C@@H]3C[C@@H]3F)ccc12]

To a solution of 5-chloro-3-(6-chloro-1-{[2-(trimethylsilyl)ethoxy]methyl}pyrrolo[2,3-b]pyridin-3-yl)-2,4-dimethoxypyridine (300.0 mg, 0.66 mmol) in dioxane (10.0 mL) was added (1S,2S)-2-fluorocyclopropane-1-carboxamide (0.3 g, 3.30 mmol), Pd₂(dba)₃ (0.1 g, 0.13 mmol), K₂CO₃ (0.3 g, 2.64 mmol) and BrettPhos (0.1 g, 0.26 mmol) at room temperature under N₂. The resulting mixture was stirred at 100° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (3/1, v/v) to afford (1S,2S)—N-[3-(5-chloro-2,4-dimethoxypyridin-3-yl)-1-{[2-(trimethylsilyl)ethoxy]methyl}pyrrolo[2,3-b]pyridin-6-yl]-2-fluorocyclopropane-1-carboxamide (150.0 mg, 43%) as a yellow solid. LCMS (ESI, m/z): [M+H]⁺=521.2

### Step 5: Synthesis of (1S,2S)—N-[3-(5-chloro-2,4-dimethoxypyridin-3-yl)-1H-pyrrolo[2,3-b]pyridin-6-yl]-2-fluorocyclopropane-1-carboxamide (Compound 231)

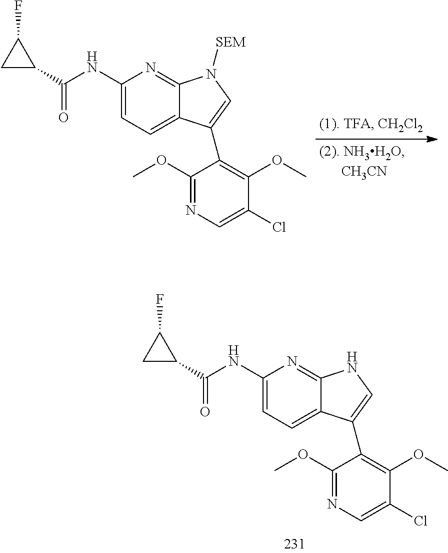 [SMILES: COc1ncc(Cl)c(OC)c1-c1cn(COCC[Si](C)(C)C)c2nc(NC(=O)[C@@H]3C[C@@H]3F)ccc12 ; COc1ncc(Cl)c(OC)c1-c1c[nH]c2nc(NC(=O)[C@@H]3C[C@@H]3F)ccc12]

To a solution of (1S,2S)—N-[3-(5-chloro-2,4-dimethoxypyridin-3-yl)-1-{[2-(trimethylsilyl)ethoxy]methyl}pyrrolo[2,3-b]pyridin-6-yl]-2-fluorocyclopropane-1-carboxamide (150.0 mg, 0.29 mmol) in DCM (2.0 mL) was added TFA (1.0 mL) at room temperature. The resulting mixture was stirred at room temperature for 2 h. The resulting mixture was concentrated under reduced pressure. To the above residue was added NH₃·H₂O (2.0 mL) and ACN (1.0 mL) at room temperature. The resulting mixture was stirred at room temperature for additional 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions (Column: XBridge Shield RP18 OBD Column, 19×250 mm, 10 m; Mobile Phase A: Water (10 mmol/L NH₄HCO₃), Mobile Phase B: ACN; Flow rate: 25 mL/min; Gradient: 45% B to 60% B in 8 min, Wave Length: 254 nm) to afford (1S,2S)—N-[3-(5-chloro-2,4-dimethoxypyridin-3-yl)-1H-pyrrolo[2,3-b]pyridin-6-yl]-2-fluorocyclopropane-1-carboxamide (Compound 231) (10.6 mg, 9%) as a white solid. LCMS (ESI, m/z): [M+H]⁺=391.1. ¹H NMR (400 MHz, DMSO-d₆): δ 11.75 (s, 1H), 10.64 (s, 1H), 8.21 (s, 1H), 7.87 (d, J=8.4 Hz, 1H), 7.72 (d, J=8.4 Hz, 1H), 7.54 (s, 1H), 5.01-4.84 (m, 1H), 3.84 (s, 3H), 3.46 (s, 3H), 2.29-2.18 (m, 1H), 1.69-1.63 (m, 1H), 1.19-1.08 (m, 1H).

### Example S210: Synthesis of (1R,2R)—N-[3-(2,4-dimethoxypyridin-3-yl)-1H-pyrrolo[2,3-b]pyridin-6-yl]-2-[(dimethylamino)methyl]cyclopropane-1-carboxamide and (1S,2S)—N-[3-(2,4-dimethoxypyridin-3-yl)-1H-pyrrolo[2,3-b]pyridin-6-yl]-2-[(dimethylamino)methyl]cyclopropane-1-carboxamide (Compound 232 and Compound 233)

### Step 1: Synthesis of 3-(6-chloro-1-{[2-(trimethylsilyl)ethoxy]methyl}pyrrolo[2,3-b]pyridin-3-yl)-2,4-dimethoxypyridine

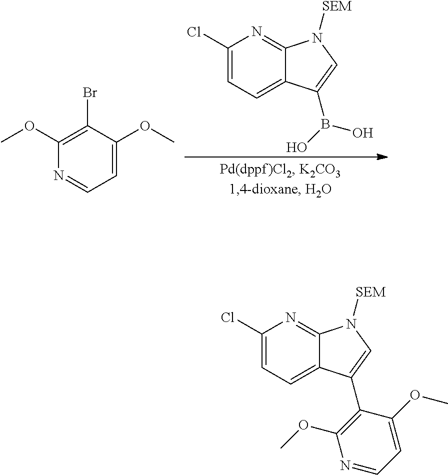

To a solution of 3-bromo-2,4-dimethoxypyridine (2.0 g, 9.17 mmol) in 1,4-dioxane (20.0 mL)/H₂O (5.0 mL) was added 6-chloro-1-{[2-(trimethylsilyl)ethoxy]methyl}pyrrolo[2,3-b]pyridin-3-ylboronic acid (3.6 g, 11.00 mmol), K₂CO₃ (3.8 g, 27.52 mmol) and Pd(dppf)Cl₂ (671.1 mg, 0.92 mmol) at room temperature under N₂. The resulting mixture was stirred at 80° C. for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford 3-(6-chloro-1-{[2-(trimethylsilyl)ethoxy]methyl}pyrrolo[2,3-b]pyridin-3-yl)-2,4-dimethoxypyridine (1.2 g, 31%) as a brown oil. LCMS (ESI, m/z): [M+H]⁺=420.1.

### Step 2: Synthesis of 3-(2,4-dimethoxypyridin-3-yl)-1-{[2-(trimethylsilyl)ethoxy]methyl}pyrrolo[2,3-b]pyridin-6-amine

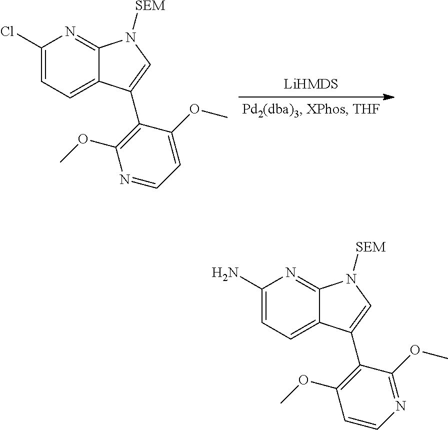 [SMILES: COc1ccnc(OC)c1-c1cn(COCC[Si](C)(C)C)c2nc(Cl)ccc12 ; COc1ccnc(OC)c1-c1cn(COCC[Si](C)(C)C)c2nc(N)ccc12]

To a solution of 3-(6-chloro-1-{[2-(trimethylsilyl)ethoxy]methyl}pyrrolo[2,3-b]pyridin-3-yl)-2,4-dimethoxypyridine (1.0 g, 2.38 mmol) in THF (40.0 mL) was added XPhos (454.0 mg, 0.95 mmol), Pd₂(dba)₃ (436.1 mg, 0.48 mmol) and LiHMDS (4.7 mL, 1 mol/L) at room temperature under N₂. The resulting mixture was stirred at 60° C. for 1 h. After the reaction was completed, the reaction mixture was quenched with NH₄Cl (aq) at room temperature. The resulting mixture was concentrated under reduced pressure. The residue was purified by flash column chromatography with petroleum ether/ethyl acetate (1/1, v/v) to afford 3-(2,4-dimethoxypyridin-3-yl)-1-{[2-(trimethylsilyl)ethoxy]methyl}pyrrolo[2,3-b]pyridin-6-amine (800.0 mg, 83%) as a yellow solid. LCMS (ESI, m/z): [M+H]⁺=401.2.

### Step 3: Synthesis of Methyl (trans)-2-{[3-(2,4-dimethoxypyridin-3-yl)-1-{[2-(trimethylsilyl)ethoxy]methyl}pyrrolo[2,3-b]pyridin-6-yl]carbamoyl}cyclopropane-1-carboxylate

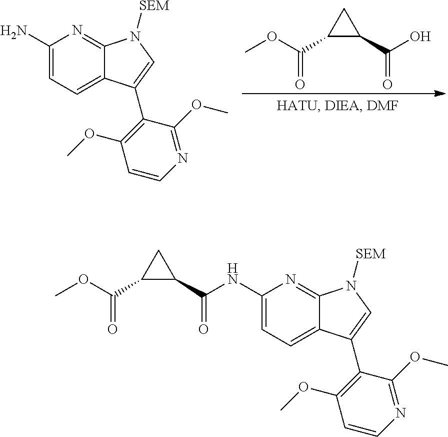

To a solution of 3-(2,4-dimethoxypyridin-3-yl)-1-{[2-(trimethylsilyl)ethoxy]methyl}pyrrolo[2,3-b]pyridin-6-amine (750.0 mg, 1.87 mmol) in DMF (20.0 mL) was added trans-2-(methoxycarbonyl)cyclopropane-1-carboxylic acid (323.2 mg, 2.24 mmol), DIEA (363.0 mg, 2.81 mmol) and HATU (1.1 g, 2.81 mmol) at 0° C. The resulting mixture was stirred at room temperature for 1 h. After the reaction was completed, the resulting mixture was diluted with water and extracted with ethyl acetate. The combined organic layers were washed with brine, dried over anhydrous sodium sulfate and filtered. The filtrate was concentrated under reduced pressure. The residue was purified by flash column chromatography with petroleum ether/ethyl acetate (2/1, v/v) to afford methyl (trans)-2-{[3-(2,4-dimethoxypyridin-3-yl)-1-{[2-(trimethylsilyl)ethoxy]methyl}pyrrolo[2,3-b]pyridin-6-yl]carbamoyl}cyclopropane-1-carboxylate (860.0 mg, 87%) as a brown oil. LCMS (ESI, m/z): [M+H]⁺=527.2.

### Step 4: Synthesis of trans-N-[3-(2,4-dimethoxypyridin-3-yl)-1-{[2-(trimethylsilyl)ethoxy]methyl}pyrrolo[2,3-b]pyridin-6-yl]-2-(hydroxymethyl)cyclopropane-1-carboxamide

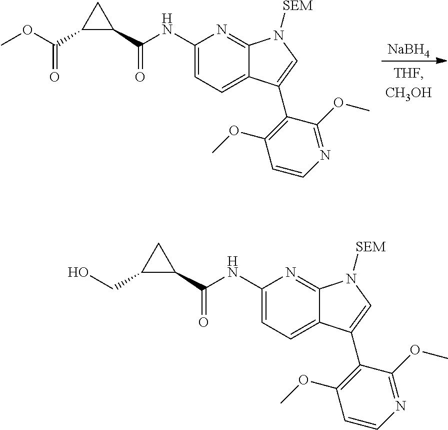

To a solution of methyl (trans)-2-{[3-(2,4-dimethoxypyridin-3-yl)-1-{[2-(trimethylsilyl)ethoxy]methyl}pyrrolo[2,3-b]pyridin-6-yl]carbamoyl}cyclopropane-1-carboxylate (850.0 mg, 1.61 mmol) in THF (20.0 mL)/MeOH (30.0 mL) was added NaBH₄ (3.1 g, 80.70 mmol) at room temperature. The resulting mixture was stirred at room temperature for 1 h. After the reaction was completed, the reaction mixture was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/99, v/v) to afford trans-N-[3-(2,4-dimethoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (530.0 mg, 65%) as a brown solid. LCMS (ESI, m/z): [M+H]⁺=499.2.

### Step 5: Synthesis of trans-N-[3-(2,4-dimethoxypyridin-3-yl)-1-{[2-(trimethylsilyl)ethoxy]methyl}pyrrolo[2,3-b]pyridin-6-yl]-2-formylcyclopropane-1-carboxamide

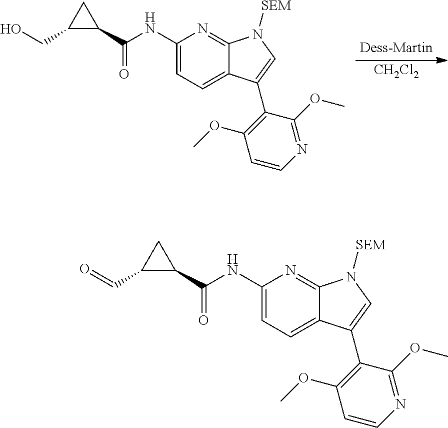 [SMILES: COc1ccnc(OC)c1-c1cn(COCC[Si](C)(C)C)c2nc(NC(=O)[C@@H]3C[C@H]3CO)ccc12 ; "CH2Cl2" ; COc1ccnc(OC)c1-c1cn(COCC[Si](C)(C)C)c2nc(NC(=O)[C@@H]3C[C@H]3C=O)ccc12]

To a solution of trans-N-[3-(2,4-dimethoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (500.0 mg, 1.00 mmol) in CH₂Cl₂ (20.0 mL) was added Dess-Martin (637.9 mg, 1.50 mmol) at 0° C. The resulting mixture was stirred at room temperature for 1 h. After the reaction was completed, the resulting mixture was diluted with water and extracted with CH₂Cl₂. The combined organic layer was washed with brine, dried over anhydrous sodium sulfate and filtered. The filtrate was concentrated under reduced pressure to afford trans-N-[3-(2,4-dimethoxypyridin-3-yl)-1-{[2-(trimethylsilyl)ethoxy]methyl}pyrrolo[2,3-b]pyridin-6-yl]-2-formylcyclopropane-1-carboxamide (495.0 mg, crude) as a yellow solid. LCMS (ESI, m/z): [M+H]⁺=497.2.

### Step 6: Synthesis of trans-N-(3-(2,4-dimethoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide

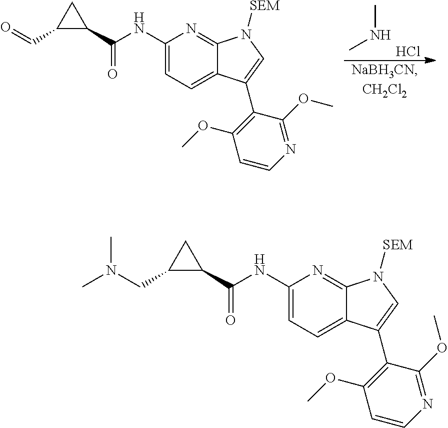

To a solution of trans-N-[3-(2,4-dimethoxypyridin-3-yl)-1-{[2-(trimethylsilyl)ethoxy]methyl}pyrrolo[2,3-b]pyridin-6-yl]-2-formylcyclopropane-1-carboxamide (495.0 mg, 0.99 mmol) in DCM (20.0 mL) was added dimethylamine hydrochloride (245.0 mg, 3.02 mmol) and NaBH₃CN (68.5 mg, 3.02 mmol) at room temperature. The resulting mixture was stirred at room temperature for 2 h. After the reaction was completed, the reaction was quenched with water at room temperature. The resulting mixture was diluted with water and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford trans-N-(3-(2,4-dimethoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (170.0 mg, 32%) as a yellow solid. LCMS (ESI, m/z): [M+H]⁺=526.3.

### Step 7: Synthesis of trans-N-(3-(2,4-dimethoxypyridin-3-yl)-1H-pyrrolo[2,3-b]pyridin-6-yl)-2-((dimethylamino)methyl)cyclopropane-1-carboxamide

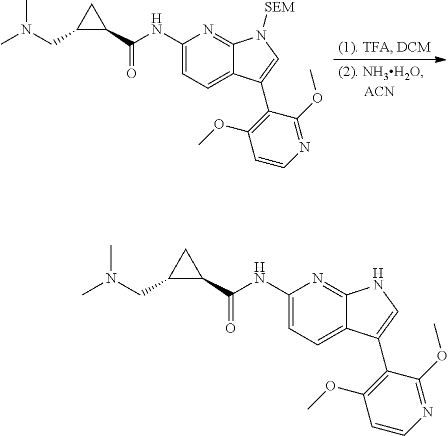 [SMILES: COc1ccnc(OC)c1-c1cn(COCC[Si](C)(C)C)c2nc(NC(=O)[C@@H]3C[C@H]3CN(C)C)ccc12 ; COc1ccnc(OC)c1-c1c[nH]c2nc(NC(=O)[C@@H]3C[C@H]3CN(C)C)ccc12]

To a solution of trans-N-(3-(2,4-dimethoxypyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (150.0 mg, 0.29 mmol) in CH₂Cl₂ (8.0 mL) was added TFA (8.0 mL) at room temperature. The resulting mixture was stirred at room temperature for 1 h. The resulting mixture was concentrated under reduced pressure. To the residue in ACN (5.0 mL) was added NH₃·H₂O (5.0 mL) at room temperature. The resulting mixture was stirred at room temperature for another 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (66/34, v/v) to afford trans-N-(3-(2,4-dimethoxypyridin-3-yl)-1H-pyrrolo[2,3-b]pyridin-6-yl)-2-((dimethylamino)methyl)cyclopropane-1-carboxamide (80.0 mg, 70%) as a white solid. LCMS (ESI, m/z): [M+H]⁺=396.1.

### Step 8: Synthesis of (1R,2R)—N-[3-(2,4-dimethoxypyridin-3-yl)-1H-pyrrolo[2,3-b]pyridin-6-yl]-2-[(dimethylamino)methyl]cyclopropane-1-carboxamide and (1S,2S)—N-[3-(2,4-dimethoxypyridin-3-yl)-1H-pyrrolo[2,3-b]pyridin-6-yl]-2-[(dimethylamino)methyl]cyclopropane-1-carboxamide (Compound 232 and Compound 233)

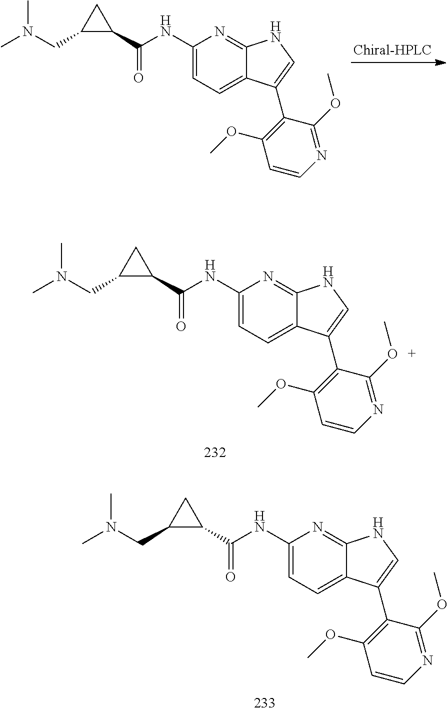 [SMILES: COc1ccnc(OC)c1-c1c[nH]c2nc(NC(=O)[C@@H]3C[C@H]3CN(C)C)ccc12 ; COc1ccnc(OC)c1-c1c[nH]c2nc(NC(=O)[C@@H]3C[C@H]3CN(C)C)ccc12 ; COc1ccnc(OC)c1-c1c[nH]c2nc(NC(=O)[C@H]3C[C@@H]3CN(C)C)ccc12]

The product trans-N-(3-(2,4-dimethoxypyridin-3-yl)-1H-pyrrolo[2,3-b]pyridin-6-yl)-2-((dimethylamino)methyl)cyclopropane-1-carboxamide (80.0 mg, 0.20 mmol) was separated by Prep-Chiral—HPLC with the following conditions (Column: CHIRAL ART Cellulose-SC, 2×25 cm, 5 m; Mobile Phase A: Hex (0.5% 2M NH₃-MeOH)—HPLC, Mobile Phase B: MeOH: EtOH=1:1—HPLC; Flow rate: 20 mL/min; Gradient: 30% B to 30% B in 14.5 min; Wave Length: 220/254 nm) to afford N-[3-(2,4-dimethoxypyridin-3-yl)-1H-pyrrolo[2,3-b]pyridin-6-yl]-2-[(dimethylamino)methyl]cyclopropane-1-carboxamide Enantiomer 1 (8.7 mg, 21%) as a white solid and N-[3-(2,4-dimethoxypyridin-3-yl)-1H-pyrrolo[2,3-b]pyridin-6-yl]-2-[(dimethylamino)methyl]cyclopropane-1-carboxamide Enantiomer 2 (7.8 mg, 20%) as a white solid. The absolute stereochemistry of Enantiomers 1 and 2 was not assigned. The two enantiomeric structures that could be obtained from chiral separation of the enantiomeric mixture as described above are shown as Compounds 232 and 233 in Table 1.

N-[3-(2,4-dimethoxypyridin-3-yl)-1H-pyrrolo[2,3-b]pyridin-6-yl]-2-[(dimethylamino)methyl]cyclopropane-1-carboxamide Enantiomer 1: Retention Time 1=10.43 min; LCMS (ESI, m/z): [M+H]⁺=396.1. ¹H NMR (400 MHz, DMSO-d₆): δ 11.51 (s, 1H), 10.51 (s, 1H), 8.07-8.02 (m, 1H), 7.83 (d, J=8.8 Hz, 1H), 7.60-7.55 (m, 1H), 7.37 (d, J=2.4 Hz, 1H), 6.89-6.82 (m, 1H), 3.98-3.80 (m, 6H), 2.35-2.27 (m, 1H), 2.19-2.15 (m, 7H), 1.96-1.89 (m, 1H), 1.36-1.28 (m, 1H), 1.11-1.04 (m, 1H), 0.72-0.68 (m, 1H).

N-[3-(2,4-dimethoxypyridin-3-yl)-1H-pyrrolo[2,3-b]pyridin-6-yl]-2-[(dimethylamino)methyl]cyclopropane-1-carboxamide Enantiomer 2: Retention Time 2=13.15 min; LCMS (ESI, m/z): [M+H]⁺=396.2. ¹H NMR (400 MHz, DMSO-d₆): δ 11.50 (s, 1H), 10.51 (s, 1H), 8.07 (d, J=5.6 Hz, 1H), 7.83 (d, J=8.8 Hz, 1H), 7.59 (d, J=8.8 Hz, 1H), 7.37 (d, J=2.4 Hz, 1H), 6.88 (d, J=6.0 Hz, 1H), 3.99-3.72 (m, 6H), 2.35-2.27 (m, 1H), 2.19-2.14 (m, 7H), 1.90-1.85 (m, 1H), 1.34-1.24 (m, 1H), 1.06-1.03 (m, 1H), 0.72-0.70 (m, 1H).

### Example S211: Synthesis of (1S,2S)—N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide and (1R,2R)—N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide (Compound 234 and Compound 235)

### Step 1: Synthesis of Trans-N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-[(dimethylamino)methyl]cyclopropane-1-carboxamide

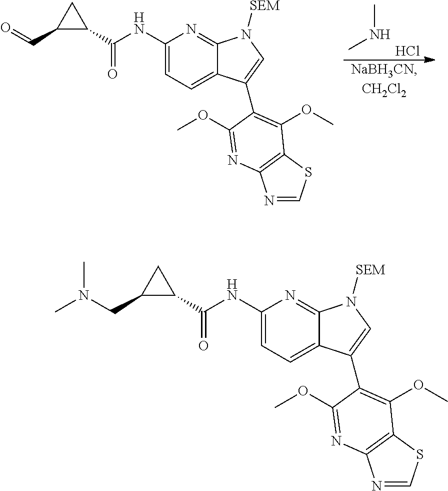

To a solution of trans-N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-formylcyclopropane-1-carboxamide (400.0 mg, 0.72 mmol) in CH₂Cl₂ (4.0 mL) was added dimethylamine hydrochloride (176.7 mg, 2.17 mmol) and NaBH₃CN (136.2 mg, 2.17 mmol) at room temperature. The resulting mixture was stirred at room temperature for 0.5 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash chromatography with CH₂Cl₂/CH₃OH (12/1, v/v)) to afford trans-N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-[(dimethylamino)methyl]cyclopropane-1-carboxamide (300.0 mg, 75%) as a brown solid. LCMS (ESI, m/z): [M+H]⁺=583.2.

### Step 2: Synthesis of Trans-N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide

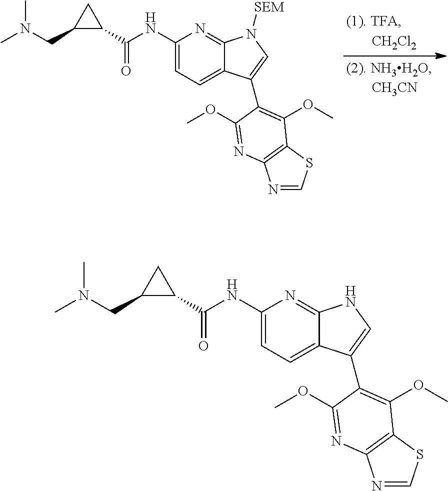

To a solution of trans-N-(3-{5,7-dimethoxy-[1,3]thiazolo[4,5-b]pyridin-6-yl}-1-{[2-(trimethylsilyl)ethoxy]methyl}pyrrolo[2,3-b]pyridin-6-yl)-2-[(dimethylamino)methyl]cyclopropane-1-carboxamide (300.0 mg, 0.52 mmol) in CH₂Cl₂ (3.0 mL) was added TFA (3.0 mL) at room temperature. The resulting mixture was stirred at room temperature for 2 h. The resulting mixture was concentrated under reduced pressure. To the residue in CH₃CN (3.0 mL) was added NH₃·H₂O (3.0 mL) at room temperature. The resulting mixture was stirred at room temperature for additional 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with H₂O/ACN (46/54, v/v) to afford trans-N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide (60.0 mg, 25%) as a green solid. LCMS (ESI, m/z): [M+H]⁺=453.2.

### Step 3: Synthesis of (1S,2S)—N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide and (1R,2R)—N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide (Compound 234 and Compound 235)

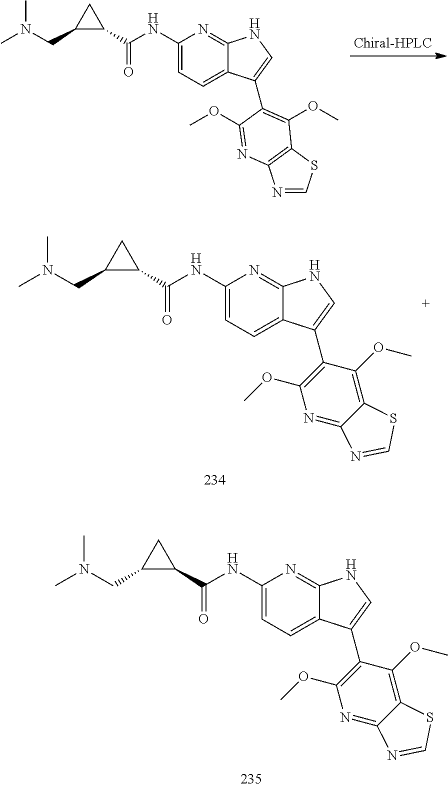

The product of trans-N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide (60.0 mg, 0.13 mmol) was separated by Prep-Chiral-HPLC with the following conditions (Column: CHIRAL ART Cellulose-SC, 2×25 cm, 5 m; Mobile Phase A: Hex: DCM=3:1 (0.5% 2M NH₃-MeOH)—HPLC, Mobile Phase B: EtOH—HPLC; Flow rate: 20 mL/min; Gradient: 30% B to 30% B in 13 min; Wave Length: 220/254 nm) to afford N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide Enantiomer 1 (17.6 mg, 58%) as a green solid and N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide Enantiomer 2 (18.3 mg, 60%) as a green solid. The absolute stereochemistry of Enantiomers 1 and 2 was not assigned. The two enantiomeric structures that could be obtained from chiral separation of the enantiomeric mixture as described above are shown as Compounds 234 and 235 in Table 1.

N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide Enantiomer 1: Retention Time 1: 8.92 min; LCMS (ESI, m/z): [M+H]⁺=453.1. ¹H NMR (400 MHz, DMSO-d₆): δ 11.66 (d, J=1.6 Hz, 1H), 10.58 (s, 1H), 9.57 (s, 1H), 7.86 (d, J=8.4 Hz, 1H), 7.69 (d, J=8.4 Hz, 1H), 7.48 (d, J=2.4 Hz, 1H), 3.91-3.87 (m, 6H), 2.33-2.15 (m, 8H), 1.91-1.85 (m, 1H), 1.36-1.32 (m, 1H), 1.07-1.01 (m, 1H), 0.72-0.65 (m, 1H).

N-(3-{5,7-dimethoxy-[1,3]thiazolo[4,5-b]pyridin-6-yl}-1H-pyrrolo[2,3-b]pyridin-6-yl)-2-[(dimethylamino)methyl]cyclopropane-1-carboxamide Enantiomer 2: Retention Time 2: 12.18 min; LCMS (ESI, m/z): [M+H]⁺=453.1. ¹H NMR (400 MHz, DMSO-d₆): δ 11.66 (d, J=1.6 Hz, 1H), 10.59 (s, 1H), 9.57 (s, 1H), 7.86 (d, J=8.4 Hz, 1H), 7.69 (d, J=8.4 Hz, 1H), 7.48 (d, J=2.4 Hz, 1H), 3.91 (s, 6H), 2.39-2.20 (m, 8H), 1.93-1.88 (m, 1H), 1.38-1.33 (m, 1H), 1.08-1.05 (m, 1H), 0.76-0.65 (m, 1H).

### Example S212: Synthesis of (1S,2S)—N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide and (1R,2R)—N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide (Compound 236 and Compound 237)

### Step 1: Synthesis of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide

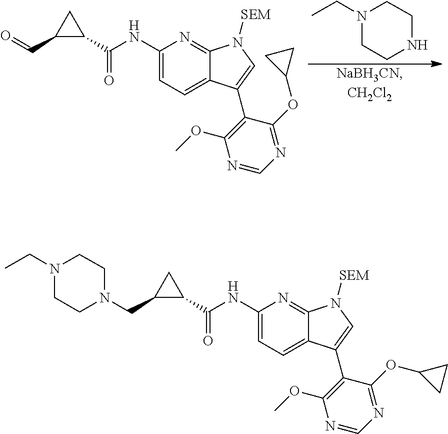

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-formylcyclopropane-1-carboxamide (170.0 mg, 0.33 mmol) in DCM (15.0 mL) was added 1-ethylpiperazine (111.2 mg, 0.98 mmol) and NaBH₃CN (61.2 mg, 0.98 mmol) at room temperature. The resulting mixture was stirred at room temperature for 2 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with CH₂Cl₂/MeOH (5/1, v/v) to afford trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide (100.0 mg, 49%) as a yellow oil. LCMS (ESI, m/z): [M+H]⁺=622.3.

### Step 2: Synthesis of trans-N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide

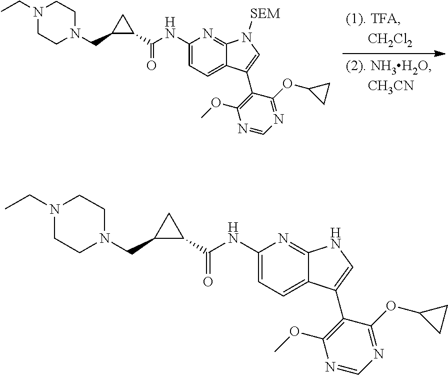 [SMILES: CCN1CCN(C[C@H]2C[C@@H]2C(=O)Nc2ccc3c(-c4c(OC)ncnc4OC4CC4)cn(COCC[Si](C)(C)C)c3n2)CC1 ; CCN1CCN(C[C@H]2C[C@@H]2C(=O)Nc2ccc3c(-c4c(OC)ncnc4OC4CC4)c[nH]c3n2)CC1]

To a solution of trans-N-[3-(4-cyclopropoxy-6-methoxypyrimidin-5-yl)-1-{[2-(trimethylsilyl)ethoxy]methyl}pyrrolo[2,3-b]pyridin-6-yl]-2-[(4-ethylpiperazin-1-yl)methyl]cyclopropane-1-carboxamide (300.0 mg, 0.48 mmol) in CH₂Cl₂ (6.0 mL) was added TFA (6.0 mL) at room temperature. The resulting mixture was stirred at room temperature for 4 h. The resulting mixture was concentrated under reduced pressure. To the residue in CH₃CN (6.0 mL) was added NH₃·H₂O (6.0 mL) at room temperature. The resulting mixture was stirred at room temperature for 16 h. After the reaction was completed, the resulting mixture was concentrated under reduced pressure. The resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with ACN/H₂O (1/1, v/v) to afford trans-N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide (100.0 mg, 42%) as a white solid. LCMS (ESI, m/z): [M+H]⁺=492.3.

### Step 3: Synthesis of (1S,2S)—N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide and (1R,2R)—N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide (Compound 236 and Compound 237)

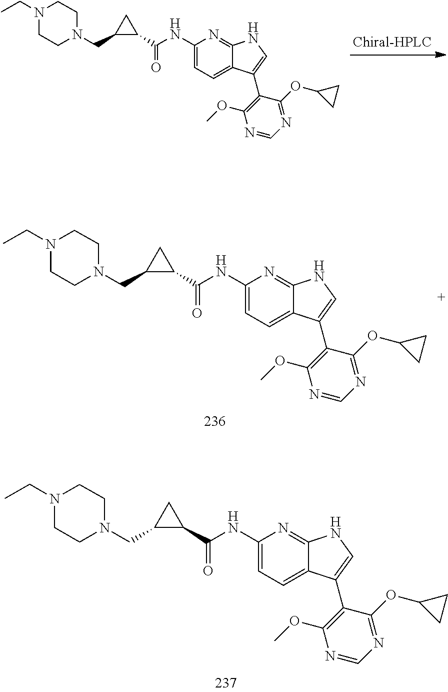 [SMILES: CCN1CCN(C[C@H]2C[C@@H]2C(=O)Nc2ccc3c(-c4c(OC)ncnc4OC4CC4)c[nH]c3n2)CC1 ; CCN1CCN(C[C@H]2C[C@@H]2C(=O)Nc2ccc3c(-c4c(OC)ncnc4OC4CC4)c[nH]c3n2)CC1 ; CCN1CCN(C[C@@H]2C[C@H]2C(=O)Nc2ccc3c(-c4c(OC)ncnc4OC4CC4)c[nH]c3n2)CC1]

The trans-N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide (100.0 mg, 0.20 mmol) was separated by Prep-Chiral-HPLC with the following conditions Column: Column (CHIRAL ART Cellulose-SC, 2×25 cm, 5 m; Mobile Phase A: Hex (0.5% 2M NH₃-MeOH)—HPLC, Mobile Phase B: EtOH—HPLC; Flow rate: 20 mL/min; Gradient: 20% B to 20% B in 17 min; Wave Length: 220/254 nm) to afford N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide Enantiomer 1 (21.5 mg, 42%) as a white solid and N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide Enantiomer 2 (20.6 mg, 41%) as a white solid. The absolute stereochemistry of Enantiomers 1 and 2 was not assigned. The two enantiomeric structures that could be obtained from chiral separation of the enantiomeric mixture as described above are shown as Compounds 236 and 237 in Table 1.

N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide Enantiomer 1: Retention Time 1: 18.75 min; LCMS (ESI, m/z): [M+H]⁺=492.3. ¹H NMR (400 MHz, DMSO-d₆): δ 11.62 (s, 1H), 10.56 (s, 1H), 8.49 (s, 1H), 7.86 (d, J=8.4 Hz, 1H), 7.65 (d, J=8.4 Hz, 1H), 7.44 (d, J=2.4 Hz, 1H), 4.39-4.34 (m, 1H), 3.92 (s, 3H), 2.51-2.21 (m, 10H), 1.91-1.89 (m, 1H), 1.34-1.32 (m, 1H), 1.06-0.96 (m, 4H), 0.78-0.62 (m, 5H).

N-[3-(4-cyclopropoxy-6-methoxypyrimidin-5-yl)-1H-pyrrolo[2,3-b]pyridin-6-yl]-2-[(4-ethylpiperazin-1-yl)methyl]cyclopropane-1-carboxamide Enantiomer 2: Retention Time 2: 14.10 min; LCMS (ESI, m/z): [M+H]⁺=492.3. ¹H NMR (400 MHz, DMSO-d₆): δ 11.62 (s, 1H), 10.56 (s, 1H), 8.49 (s, 1H), 7.86 (d, J=8.8 Hz, 1H), 7.65 (d, J=8.8 Hz, 1H), 7.44 (d, J=2.4 Hz, 1H), 4.39-4.34 (m, 1H), 3.92 (s, 3H), 2.51-2.21 (m, 7H), 1.91-1.89 (m, 1H), 1.34-1.32 (m, 1H), 1.06-0.96 (m, 4H), 0.78-0.62 (m, 5H).

### Example S213: Synthesis of (1R,2R)—N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide and (1S,2S)—N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide (Compound 238 and Compound 239)

### Step 1: Synthesis of 4-chloro-2-(methoxy-d3)-3-nitropyridine

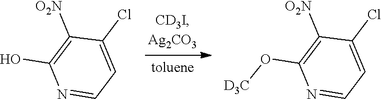 [SMILES: O=[N+]([O-])c1c(Cl)ccnc1O ; [2H]C([2H])([2H])Oc1nccc(Cl)c1[N+](=O)[O-]]

To a solution of 4-chloro-3-nitropyridin-2-ol (12.0 g, 68.97 mmol) in toluene (150.0 mL) was added CD₃I (20.0 g, 137.94 mmol) and Ag₂CO₃ (28.6 g, 103.46 mmol) at room temperature. The resulting mixture was stirred at 80° C. for 2 h. After the reaction was completed, the resulting mixture was concentrated under reduced pressure. The residue was purified by flash column chromatography with petroleum ether/ethyl acetate (66/34, v/v) to afford 4-chloro-2-(methoxy-d3)-3-nitropyridine (8.0 g, 60%) as a white solid. LCMS (ESI, m/z): [M+H]⁺=192.0.

### Step 2: Synthesis of 4-cyclopropoxy-2-(methoxy-d3)-3-nitropyridine

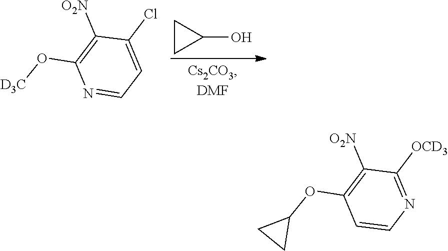

To a solution of 4-chloro-2-(methoxy-d3)-3-nitropyridine (8.0 g, 41.76 mmol) in DMF (100.0 mL) was added cyclopropanol (3.6 g, 62.63 mmol) and Cs₂CO₃ (20.4 g, 62.63 mmol) at room temperature. The resulting mixture was stirred at room temperature for 3 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (74/26, v/v) to afford 4-cyclopropoxy-2-(methoxy-d3)-3-nitropyridine (4.7 g, 52%) as a yellow oil. LCMS (ESI, m/z): [M+H]⁺=214.1.

### Step 3: Synthesis of 4-cyclopropoxy-2-(methoxy-d3)pyridin-3-amine

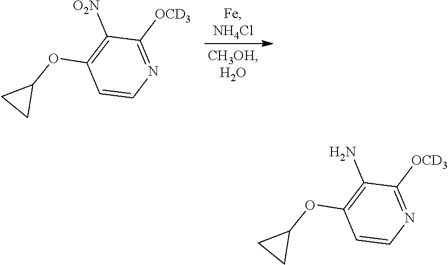

To a solution of 4-cyclopropoxy-2-(methoxy-d3)-3-nitropyridine (4.7 g, 22.04 mmol) in MeOH (30.0 mL)/H₂O (6.0 mL) was added NH₄Cl (4.7 g, 88.18 mmol) at room temperature. Then Fe (3.7 g, 66.13 mmol) was added to the mixture at 80° C. The resulting mixture was stirred at 80° C. for 3 h. After the reaction was completed, the resulting mixture was filtered. The filtrate was concentrated under reduced pressure. The residue was purified by flash column chromatography with petroleum ether/ethyl acetate (78/22, v/v) to afford 4-cyclopropoxy-2-(methoxy-d3)pyridin-3-amine (3.1 g, 76%) as a white solid. LCMS (ESI, m/z): [M+H]⁺=184.1.

### Step 4: Synthesis of 4-cyclopropoxy-3-iodo-2-(methoxy-d3)pyridine

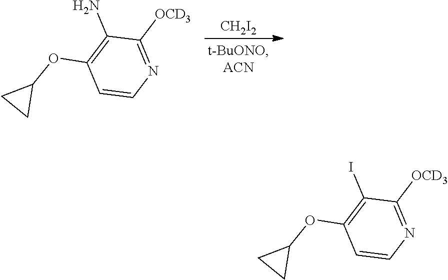

To a solution of 4-cyclopropoxy-2-(methoxy-d3)pyridin-3-amine (3.1 g, 16.92 mmol) in MeCN (35.0 mL) were added CH₂I2 (4.5 g, 16.92 mmol) and tert-butyl nitrite (5.2 g, 50.76 mmol) at room temperature. The resulting mixture was stirred at 80 C for 2 h. After the reaction was completed, the resulting mixture was concentrated under reduced pressure. The residue was purified by flash column chromatography with petroleum ether/ethyl acetate (82/18, v/v) to afford 4-cyclopropoxy-3-iodo-2-(methoxy-d3)pyridine (2.8 g, 56%) as a white solid. LCMS (ESI, m/z): [M+H]⁺=295.0.

### Step 5: Synthesis of 6-chloro-3-(4-cyclopropoxy-2-(methoxy-d3)pyridin-3-yl)-1-((2-(trimethylsilyl)ethoxy)methyl)-1H-pyrrolo[2,3-b]pyridine

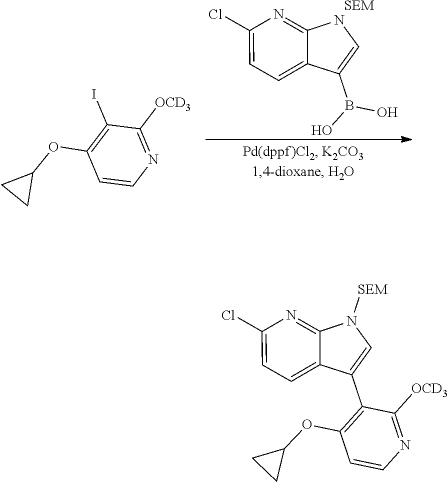

To a solution of 4-cyclopropoxy-3-iodo-2-(methoxy-d3)pyridine (2.8 g, 9.52 mmol) in 1,4-dioxane (24.0 mL) and H₂O (6.0 mL) were added (6-chloro-1-((2-(trimethylsilyl)ethoxy)methyl)-1H-pyrrolo[2,3-b]pyridin-3-yl)boronic acid (3.1 g, 9.52 mmol), K₂CO₃ (3.9 g, 28.56 mmol) and Pd(dppf)Cl₂ (1.3 g, 1.90 mmol) at room temperature under N₂. The mixture was stirred at 80° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (81/19, v/v) to afford 6-chloro-3-(4-cyclopropoxy-2-(methoxy-d3)pyridin-3-yl)-1-((2-(trimethylsilyl)ethoxy)methyl)-1H-pyrrolo[2,3-b]pyridine (2.0 g, 46%) as a yellow oil. LCMS (ESI, m/z): [M+H]⁺=449.2.

### Step 6: Synthesis of 3-(4-cyclopropoxy-2-(methoxy-d3)pyridin-3-yl)-1-((2-(trimethylsilyl)ethoxy)methyl)-1H-pyrrolo[2,3-b]pyridin-6-amine

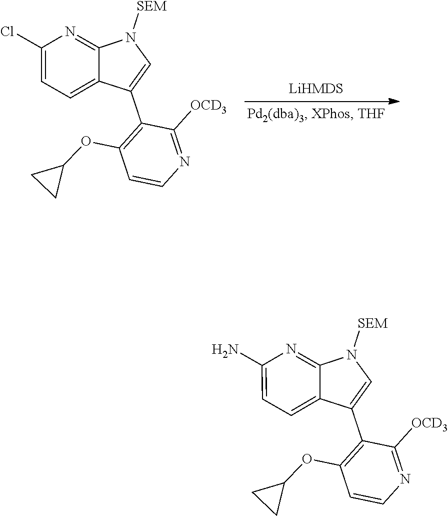

To a solution of 6-chloro-3-(4-cyclopropoxy-2-(methoxy-d3)pyridin-3-yl)-1-((2-(trimethylsilyl)ethoxy)methyl)-1H-pyrrolo[2,3-b]pyridine (2.0 g, 4.45 mmol) in THF (20.0 mL) were added Pd₂(dba)₃ (815.7 mg, 0.89 mmol), XPhos (849.3 mg, 1.78 mmol) and LiHMDS (8.9 mL, 1.0 mol/L) at room temperature under N₂. The resulting mixture was stirred at 60° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (87/13, v/v) to afford 3-(4-cyclopropoxy-2-(methoxy-d3)pyridin-3-yl)-1-((2-(trimethylsilyl)ethoxy)methyl)-1H-pyrrolo[2,3-b]pyridin-6-amine (1.2 g, 62%) as a yellow oil. LCMS (ESI, m/z): [M+H]⁺=430.2.

### Step 7: Synthesis of Methyl (trans)-2-((3-(4-cyclopropoxy-2-(methoxy-d3)pyridin-3-yl)-1-((2-(trimethylsilyl)ethoxy)methyl)-1H-pyrrolo[2,3-b]pyridin-6-yl)carbamoyl)cyclopropane-1-carboxylate

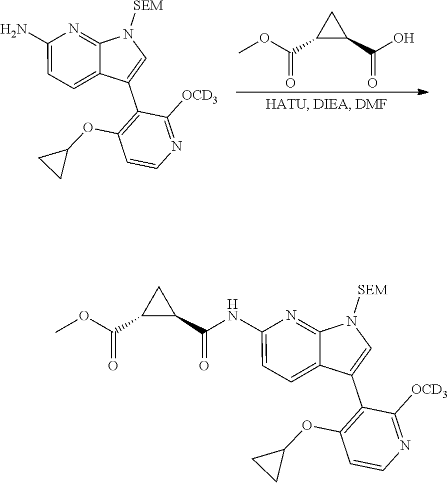

To a solution of 3-(4-cyclopropoxy-2-(methoxy-d3)pyridin-3-yl)-1-((2-(trimethylsilyl)ethoxy)methyl)-1H-pyrrolo[2,3-b]pyridin-6-amine (1.2 g, 2.79 mmol) in DMF (15.0 mL) was added trans-2-(methoxycarbonyl)cyclopropane-1-carboxylic acid (483.1 mg, 3.35 mmol), DIEA (1.8 g, 13.97 mmol) and HATU (1.6 g, 4.19 mmol) at 0 C under N₂. The resulting mixture was stirred at 0° C. for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (80/20, v/v) to afford methyl (trans)-2-((3-(4-cyclopropoxy-2-(methoxy-d3)pyridin-3-yl)-1-((2-(trimethylsilyl)ethoxy)methyl)-1H-pyrrolo[2,3-b]pyridin-6-yl)carbamoyl)cyclopropane-1-carboxylate (1.2 g, 77%) as a yellow oil. LCMS (ESI, m/z): [M+H]⁺=556.3.

### Step 8: Synthesis of Trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-(hydroxymethyl)cyclopropane-1-carboxamide

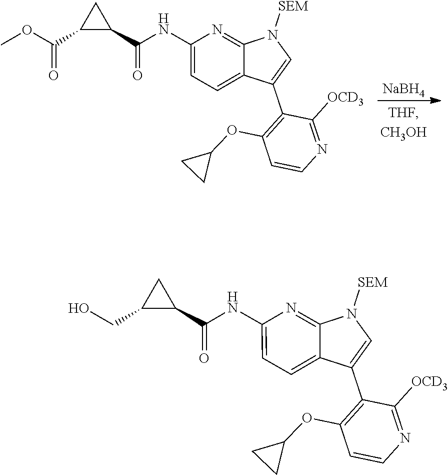

To a solution of methyl (trans)-2-((3-(4-cyclopropoxy-2-(methoxy-d3)pyridin-3-yl)-1-((2-(trimethylsilyl)ethoxy)methyl)-1H-pyrrolo[2,3-b]pyridin-6-yl)carbamoyl)cyclopropane-1-carboxylate (1.2 g, 2.16 mmol) in THF (12.0 mL)/CH₃OH (3.0 mL) was added NaBH₄ (1.6 g, 43.18 mmol) at room temperature. The resulting mixture was stirred at room temperature for 6 h. After the reaction was completed, the reaction mixture was quenched with water at 0° C. and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (55/45, v/v) to afford trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-(hydroxymethyl)cyclopropane-1-carboxamide (750.0 mg, 65%) as a white solid. LCMS (ESI, m/z): [M+H]⁺=528.3.

### Step 9: Synthesis of Trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-formylcyclopropane-1-carboxamide

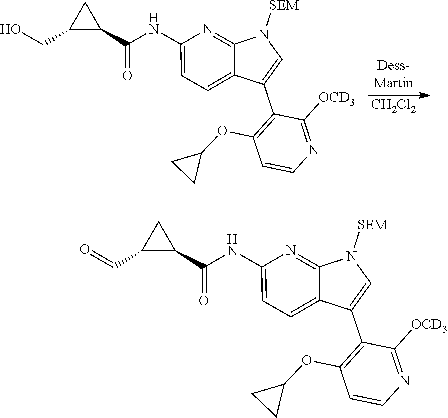

To a solution of trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-(hydroxymethyl)cyclopropane-1-carboxamide (750.0 mg, 1.42 mmol) in CH₂Cl₂ (10.0 mL) was added Dess-Martin (904.2 mg, 2.13 mmol) at 0 C under N₂. The resulting mixture was stirred at room temperature for 2 h. After the reaction was completed, the resulting mixture was diluted with water and extracted with CH₂Cl₂. The combined organic layer was washed with NaHCO₃ (aq), brine, dried over anhydrous sodium sulfate and filtered. The filtrate was concentrated under reduced pressure to afford trans-N-(3-(4-cyclopropoxy-2-(methoxy-d₃)pyridin-3-yl)-1-((2-(trimethylsilyl)ethoxy)methyl)-1H-pyrrolo[2,3-b]pyridin-6-yl)-2-formylcyclopropane-1-carboxamide (700.0 mg, crude) as a brown solid. LCMS (ESI, m/z): [M+H]⁺=526.2.

### Step 10: Synthesis of Trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide

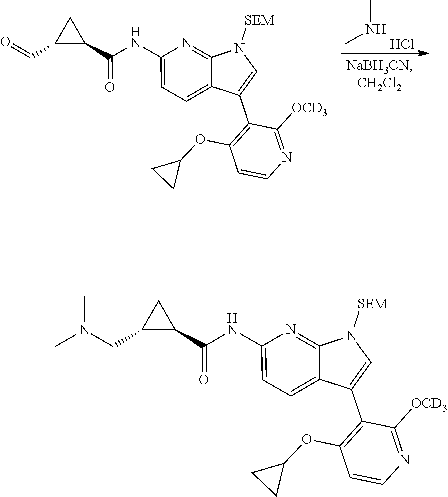

To a solution of trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-formylcyclopropane-1-carboxamide (700.0 mg, crude) in CH₂Cl₂ (10.0 mL) were added dimethylamine hydrochloride (325.7 mg, 4.00 mmol) and NaBH₃CN (251.0 mg, 4.00 mmol) at 0 C under N₂. The resulting mixture was stirred at room temperature for 2 h. After the reaction was completed, the resulting mixture was diluted with water and extracted with CH₂Cl₂. The combined organic layer was washed with brine, dried over anhydrous sodium sulfate and filtered. The filtrate was concentrated under reduced pressure. The residue was purified by flash column chromatography with petroleum CH₂Cl₂/CH₃OH (10/01, v/v) to afford trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (200.0 mg, 23%) as a yellow solid. LCMS (ESI, m/z): [M+H]⁺=555.3.

### Step 11: Synthesis of Trans-N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide

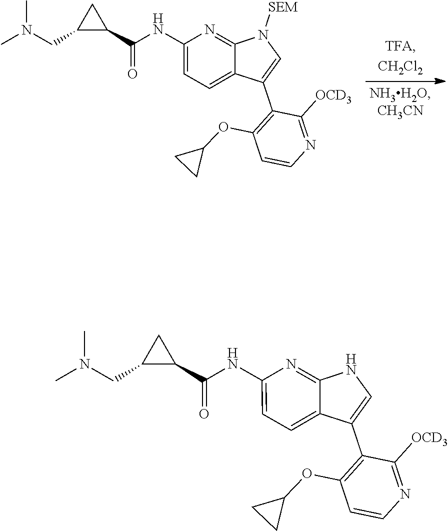

To a solution of trans-N-(3-(4-cyclopropoxy-2-(methoxy-d3)pyridin-3-yl)-1-((2-(trimethylsilyl)ethoxy)methyl)-1H-pyrrolo[2,3-b]pyridin-6-yl)-2-((dimethylamino)methyl)cyclopropane-1-carboxamide (200.0 mg, 0.36 mmol) in CH₂Cl₂ (1.5 mL) was added TFA (1.5 mL) at room temperature. The resulting mixture was stirred at room temperature for 2 h. The resulting mixture was concentrated under reduced pressure. To the residue in CH₃CN (1.5 mL) was added NH₃·H₂O (1.5 mL) at room temperature. The resulting mixture was stirred at room temperature for 2 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₃CN/H₂O (55/45, v/v) to afford trans-N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide (50.0 mg, 32%) as a yellow solid. LCMS (ESI, m/z): [M+H]⁺=425.2.

### Step 12: Synthesis of (1R,2R)—N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide and (1S,2S)—N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide (Compound 238 and Compound 239)

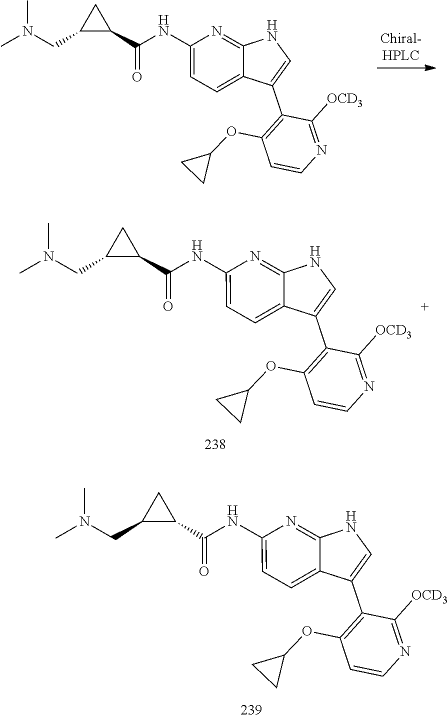

The product trans-N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide (50.0 mg, 0.12 mmol) was separated by Prep-Chiral-HPLC with the following conditions (Column: CHIRAL ART Cellulose-SC, 2×25 cm, 5 m; Mobile Phase A: Hex: DCM=3:1 (0.5% 2M NH₃-MeOH)—HPLC, Mobile Phase B: EtOH—HPLC; Flow rate: 20 mL/min; Gradient: 10% B to 10% B in 12.5 min; Wave Length: 220/254 nm) to afford N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide Enantiomer 1 (14.4 mg, 57%) as a white solid and N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide Enantiomer 2 (13.3 mg, 52%) as a white solid. The absolute stereochemistry of Enantiomers 1 and 2 was not assigned. The two enantiomeric structures that could be obtained from chiral separation of the enantiomeric mixture as described above are shown as Compounds 238 and 239 in Table 1.

N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide Enantiomer 1: Retention Time 1: 8.39 min; LCMS (ESI, m/z): [M+H]⁺=425.2. ¹H NMR (400 MHz, DMSO-d₆): δ 11.50 (d, J=2.0 Hz, 1H), 10.52 (s, 1H), 8.07 (d, J=5.6 Hz, 1H), 7.83 (d, J=8.8 Hz, 1H), 7.56 (d, J=8.4 Hz, 1H), 7.34 (d, J=2.4 Hz, 1H), 7.14 (d, J=6.0 Hz, 1H), 3.94-3.91 (m, 1H), 2.35-2.30 (m, 1H), 2.24-2.19 (m, 7H), 1.91-1.88 (m, 1H), 1.37-1.33 (m, 1H), 1.07-1.03 (m, 1H), 0.81-0.76 (m, 2H), 0.74-0.69 (m, 1H), 0.66-0.62 (m, 2H).

N-(3-(4-cyclopropoxy-2-(methoxy-d3)pyridin-3-yl)-1H-pyrrolo[2,3-b]pyridin-6-yl)-2-((dimethylamino)methyl)cyclopropane-1-carboxamide Enantiomer 2: Retention Time 2: 11.34 min; LCMS (ESI, m/z): [M+H]⁺=425.2. ¹H NMR (400 MHz, DMSO-d₆): δ 11.50 (d, J=2.0 Hz, 1H), 10.52 (s, 1H), 8.07 (d, J=5.6 Hz, 1H), 7.83 (d, J=8.8 Hz, 1H), 7.56 (d, J=8.4 Hz, 1H), 7.34 (d, J=2.4 Hz, 1H), 7.14 (d, J=6.0 Hz, 1H), 3.94-3.91 (m, 1H), 2.34-2.29 (m, 1H), 2.21-2.18 (m, 7H), 1.91-1.89 (m, 1H), 1.37-1.32 (m, 1H), 1.07-1.03 (m, 1H), 0.81-0.76 (m, 2H), 0.73-0.69 (m, 1H), 0.66-0.62 (m, 2H).

### Example S214: Synthesis of (1R,2R)—N-(3-(4-cyclopropoxy-2-methoxypyridin-3-yl)-1H-pyrrolo[2,3-b]pyridin-6-yl)-2-((methylamino)methyl)cyclopropanecarboxamide and (1S,2S)—N-(3-(4-cyclopropoxy-2-methoxypyridin-3-yl)-1H-pyrrolo[2,3-b]pyridin-6-yl)-2-((methylamino)methyl)cyclopropanecarboxamide (Compound 240 and Compound 241)

### Step 1: Synthesis of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((methylamino)methyl)cyclopropane-1-carboxamide

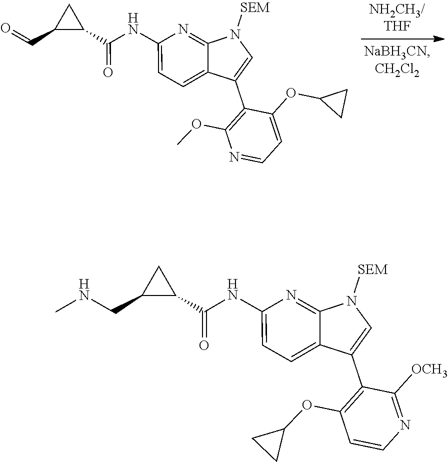 [SMILES: COc1nccc(OC2CC2)c1-c1cn(COCC[Si](C)(C)C)c2nc(NC(=O)[C@H]3C[C@@H]3C=O)ccc12 ; CNC[C@H]1C[C@@H]1C(=O)Nc1ccc2c(-c3c(OC4CC4)ccnc3OC)cn(COCC[Si](C)(C)C)c2n1]

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-formylcyclopropane-1-carboxamide (450.0 mg, 0.87 mmol) in CH₂Cl₂ (5.0 mL) was added methanamine (0.2 mL, 2 mol/L in THF) and NaBH₃CN (162.3 mg, 2.58 mmol) at room temperature. The resulting mixture was stirred at room temperature for 2 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by reversed phase flash column chromatography with water/MeOH (60/40, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((methylamino)methyl)cyclopropane-1-carboxamide (100.0 mg, 22%) as a yellow oil. LCMS (ESI, m/z): [M+H]⁺=538.3.

### Step 2: Synthesis of trans-N-(3-(4-cyclopropoxy-2-methoxypyridin-3-yl)-1H-pyrrolo[2,3-b]pyridin-6-yl)-2-((methylamino)methyl)cyclopropane-1-carboxamide

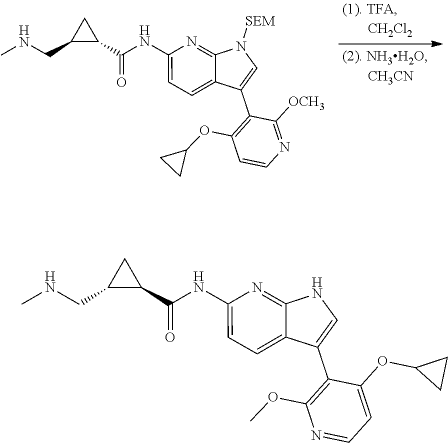 [SMILES: CNC[C@H]1C[C@@H]1C(=O)Nc1ccc2c(-c3c(OC4CC4)ccnc3OC)cn(COCC[Si](C)(C)C)c2n1 ; CNC[C@@H]1C[C@H]1C(=O)Nc1ccc2c(-c3c(OC4CC4)ccnc3OC)c[nH]c2n1]

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-((methylamino)methyl)cyclopropane-1-carboxamide (130.0 mg, 0.24 mmol) in CH₂Cl₂ (2.0 mL) was added TFA (4.0 mL) at room temperature. The resulting mixture was stirred at room temperature for 4 h. The resulting mixture was concentrated under reduced pressure. To the residue in CH₃CN (2.0 mL) was added NH₃·H₂O (4.0 mL) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reversed phase flash column chromatography with water/MeOH (0/100, v/v) to afford trans-N-(3-(4-cyclopropoxy-2-methoxypyridin-3-yl)-1H-pyrrolo[2,3-b]pyridin-6-yl)-2-((methylamino)methyl)cyclopropane-1-carboxamide (70.0 mg, 71%) as a yellow solid. LCMS (ESI, m/z): [M+H]⁺=408.2.

### Step 3: Synthesis of (1R,2R)—N-(3-(4-cyclopropoxy-2-methoxypyridin-3-yl)-1H-pyrrolo[2,3-b]pyridin-6-yl)-2-((methylamino)methyl)cyclopropanecarboxamide and (1S,2S)—N-(3-(4-cyclopropoxy-2-methoxypyridin-3-yl)-1H-pyrrolo[2,3-b]pyridin-6-yl)-2-((methylamino)methyl)cyclopropanecarboxamide (Compound 240 and Compound 241)

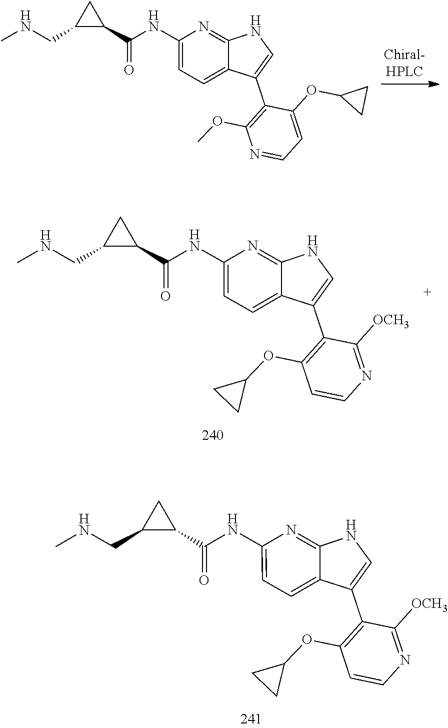 [SMILES: CNC[C@@H]1C[C@H]1C(=O)Nc1ccc2c(-c3c(OC4CC4)ccnc3OC)c[nH]c2n1 ; CNC[C@@H]1C[C@H]1C(=O)Nc1ccc2c(-c3c(OC4CC4)ccnc3OC)c[nH]c2n1 ; CNC[C@H]1C[C@@H]1C(=O)Nc1ccc2c(-c3c(OC4CC4)ccnc3OC)c[nH]c2n1]

The compound trans−-N-(3-(4-cyclopropoxy-2-methoxypyridin-3-yl)-1H-pyrrolo[2,3-b]pyridin-6-yl)-2-((methylamino)methyl)cyclopropanecarboxamide (70.0 mg, 0.17 mmol) was separated by Prep-Chiral-HPLC with the following conditions (Column: CHIRAL ART Amylose-SA, 2×25 cm, 5 um; Mobile Phase A: Hex: DCM=3: 1 (0.5% 2M NH₃-MeOH)—HPLC, Mobile Phase B: EtOH—HPLC; Flow rate: 20 mL/min; Gradient: 20% B to 20% B in 19 min; 254 nm) to afford N-(3-(4-cyclopropoxy-2-methoxypyridin-3-yl)-1H-pyrrolo[2,3-b]pyridin-6-yl)-2-((methylamino)methyl)cyclopropane-1-carboxamide Enantiomer 1 (5.5 mg, 15%) as a white solid and N-(3-(4-cyclopropoxy-2-methoxypyridin-3-yl)-1H-pyrrolo[2,3-b]pyridin-6-yl)-2-((methylamino)methyl)cyclopropane-1-carboxamide Enantiomer 2 (6.1 mg, 18%) as a white solid. The absolute stereochemistry of Enantiomers 1 and 2 was not assigned. The two enantiomeric structures that could be obtained from chiral separation of the enantiomeric mixture as described above are shown as Compounds 240 and 241 in Table 1.

N-(3-(4-cyclopropoxy-2-methoxypyridin-3-yl)-1H-pyrrolo[2,3-b]pyridin-6-yl)-2-((methylamino)methyl)cyclopropane-1-carboxamide Enantiomer 1: Retention Time 1: 22.47 min; LCMS (ESI, m/z): [M+H]⁺=408.1. ¹H NMR (400 MHz, DMSO-d₆): δ 11.55 (s, 1H), 10.49 (d, J=4.8 Hz, 1H), 8.08 (d, J=5.6 Hz, 1H), 7.83 (d, J=8.4 Hz, 1H), 7.56 (d, J=8.4 Hz, 1H), 7.35 (s, 1H), 7.14 (d, J=6.0 Hz, 1H), 4.00-3.92 (m, 1H), 3.82 (s, 3H), 2.68-2.60 (m, 1H), 2.34 (s, 3H), 1.96-1.93 (m, 1H), 1.49-1.42 (m, 1H), 1.05-1.03 (m, 1H), 0.88-0.78 (m, 3H), 0.72-0.64 (m, 2H).

N-(3-(4-cyclopropoxy-2-methoxypyridin-3-yl)-1H-pyrrolo[2,3-b]pyridin-6-yl)-2-((methylamino)methyl)cyclopropane-1-carboxamide Enantiomer 2: Retention Time 2:26.92 min; LCMS (ESI, m/z): [M+H]⁺=408.1. ¹H NMR (400 MHz, DMSO-d₆): δ 11.55 (s, 1H), 10.47 (s, 1H), 8.07 (d, J=5.6 Hz, 1H), 7.83 (d, J=8.0 Hz, 1H), 7.56 (d, J=8.4 Hz, 1H), 7.35 (s, 1H), 7.14 (d, J=5.6 Hz, 1H), 3.95-3.91 (m, 1H), 3.81 (s, 3H), 2.68-2.60 (m, 1H), 2.32 (s, 3H), 1.97-1.91 (m, 1H), 1.49-1.42 (m, 1H), 1.05-1.02 (m, 1H), 0.81-0.74 (m, 3H), 0.70-0.64 (m, 2H).

### Example S215: Synthesis of (1S,2S)—N-[3-(4-cyclobutoxy-2-methoxypyridin-3-yl)-1H-pyrrolo[2,3-b]pyridin-6-yl]-2-[(dimethylamino)methyl]cyclopropane-1-carboxamide and (1R,2R)—N-[3-(4-cyclobutoxy-2-methoxypyridin-3-yl)-1H-pyrrolo[2,3-b]pyridin-6-yl]-2-[(dimethylamino)methyl]cyclopropane-1-carboxamide (Compound 242 and Compound 243)

### Step 1: Synthesis of 4-chloro-2-methoxy-3-nitropyridine

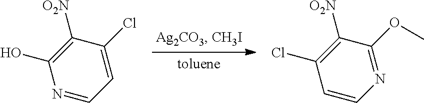

To a solution of 4-chloro-3-nitropyridin-2-ol (30.0 g, 171.88 mmol) in toluene (900.0 mL) was added Ag₂CO₃ (71.1 g, 257.82 mmol) and CH₃I (48.8 g, 343.76 mmol) at room temperature. The resulting mixture was stirred at 80° C. for 1 h. After the reaction was completed, the resulting mixture was filtered. The filtrate was concentrated under reduced pressure. The residue was purified by flash column chromatography with petroleum ether/ethyl acetate (1/9, v/v) to afford 4-chloro-2-methoxy-3-nitropyridine (18.7 g, 58%) as a yellow oil. LCMS (ESI, m/z): [M+H]⁺=189.0.

### Step 2: Synthesis of 4-cyclobutoxy-2-methoxy-3-nitropyridine

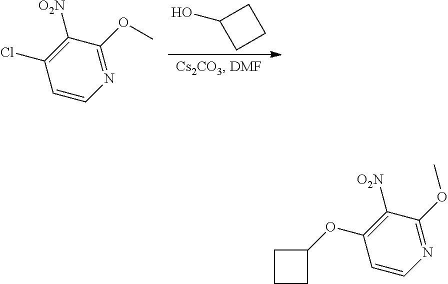

To a solution of 4-chloro-2-methoxy-3-nitropyridine (18.7 g, 99.17 mmol) in DMF (600.0 mL) was added cyclobutanol (10.7 g, 148.75 mmol) and Cs₂CO₃ (48.5 g, 148.75 mmol) at room temperature. The resulting mixture was stirred at 80° C. for 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford 4-cyclobutoxy-2-methoxy-3-nitropyridine (13.8 g, 62%) as a yellow oil. LCMS (ESI, m/z): [M+H]⁺=225.1

### Step 3: Synthesis of 4-cyclobutoxy-2-methoxypyridin-3-amine

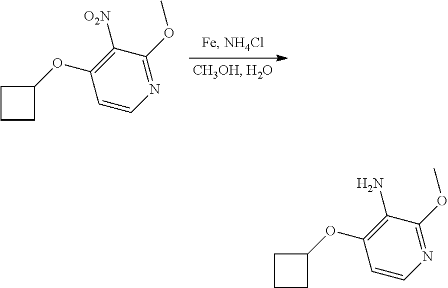 [SMILES: COc1nccc(OC2CCC2)c1[N+](=O)[O-] ; COc1nccc(OC2CCC2)c1N]

To a solution of 4-cyclobutoxy-2-methoxy-3-nitropyridine (13.8 g, 61.55 mmol) in methanol/H₂O (100.0 mL/100 mL) was added NH₄Cl (13.2 g, 246.19 mmol) and Fe (10.3 g, 184.64 mmol) at room temperature. The resulting mixture was stirred at 80° C. for 1 h. After the reaction was completed, the resulting mixture was filtered. The filtrate was concentrated under reduced pressure. The residue was purified by flash column chromatography with petroleum ether/ethyl acetate (1/10, v/v) to afford 4-cyclobutoxy-2-methoxypyridin-3-amine (8.5 g, 71%) as a yellow oil. LCMS (ESI, m/z): [M+H]⁺=195.1.

### Step 4: Synthesis of 4-cyclobutoxy-3-iodo-2-methoxypyridine

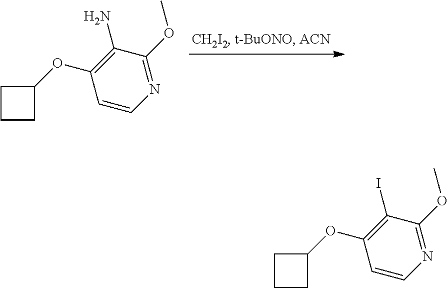

To a solution of 4-cyclobutoxy-2-methoxypyridin-3-amine (8.5 g, 43.76 mmol) in CAN (300.0 mL) was added CH₂I2 (11.7 g, 43.76 mmol) and t-BuNO₂ (20.3 g, 196.93 mmol) at room temperature. The resulting mixture was stirred at 80° C. for 1 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford 4-cyclobutoxy-3-iodo-2-methoxypyridine (4.2 g, 32%) as a brown oil. LCMS (ESI, m/z): [M+H]⁺=306.0.

### Step 5: Synthesis of 3-(6-chloro-1-{[2-(trimethylsilyl)ethoxy]methyl}pyrrolo[2,3-b]pyridin-3-yl)-4-cyclobutoxy-2-methoxypyridine

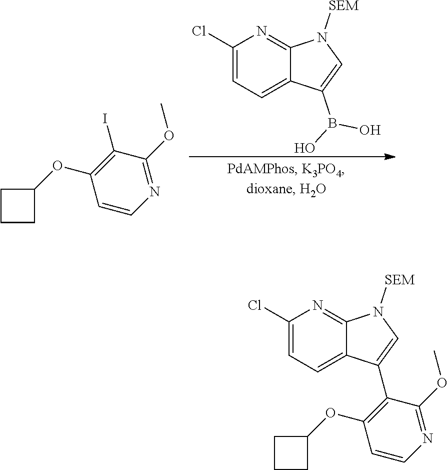 [SMILES: COc1nccc(OC2CCC2)c1I ; C[Si](C)(C)CCOCn1cc(B(O)O)c2ccc(Cl)nc21 ; COc1nccc(OC2CCC2)c1-c1cn(COCC[Si](C)(C)C)c2nc(Cl)ccc12]

To a solution of 4-cyclobutoxy-3-iodo-2-methoxypyridine (2.1 g, 10.94 mmol) in 1,4-dioxane/H₂O (100.0 mL/20.0 mL) was added 6-chloro-1-{[2-(trimethylsilyl)ethoxy]methyl}pyrrolo[2,3-b]pyridin-3-ylboronic acid (1.9 g, 5.77 mmol), K₃PO₄ (3.8 g, 17.30 mmol) and PdAMPhos (0.8 g, 1.15 mmol) at room temperature. The resulting mixture was stirred at 80° C. for 1 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/9, v/v) to afford 3-(6-chloro-1-{[2-(trimethylsilyl)ethoxy]methyl}pyrrolo[2,3-b]pyridin-3-yl)-4-cyclobutoxy-2-methoxypyridine (2.0 g, 73%) as a brown oil. LCMS (ESI, m/z): [M+H]⁺=460.2.

### Step 6: Synthesis of 3-(4-cyclobutoxy-2-methoxypyridin-3-yl)-1-{[2-(trimethylsilyl)ethoxy]methyl}pyrrolo[2,3-b]pyridin-6-amine

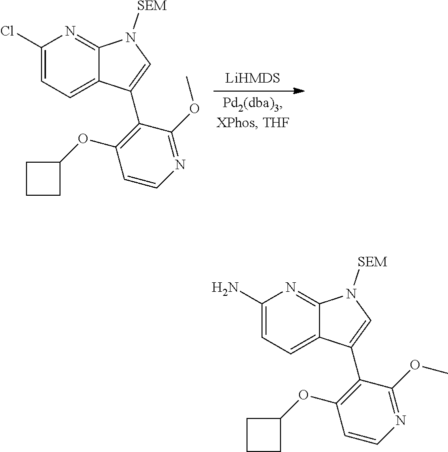 [SMILES: COc1nccc(OC2CCC2)c1-c1cn(COCC[Si](C)(C)C)c2nc(Cl)ccc12 ; COc1nccc(OC2CCC2)c1-c1cn(COCC[Si](C)(C)C)c2nc(N)ccc12]

To a solution of 3-(6-chloro-1-{[2-(trimethylsilyl)ethoxy]methyl}pyrrolo[2,3-b]pyridin-3-yl)-4-cyclobutoxy-2-methoxypyridine (2.0 g, 4.35 mmol) in THE (60.0 mL) was added Pd₂(dba)₃ (0.4 g, 0.44 mmol), XPhos (0.6 g, 1.30 mmol) and LiHMDS (13.0 mL, 1 mol/L) at room temperature under N₂. The resulting mixture was stirred at 80° C. for 1 h. After the reaction was completed, the reaction was quenched by the addition of aq. NH₄Cl at 0° C. and then extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/4, v/v) to afford 3-(4-cyclobutoxy-2-methoxypyridin-3-yl)-1-{[2-(trimethylsilyl)ethoxy]methyl}pyrrolo[2,3-b]pyridin-6-amine (1.7 g, 89%) as a yellow oil. LCMS (ESI, m/z): [M+H]⁺=441.2.

### Step 7: Synthesis of Methyl trans-2-{[3-(4-cyclobutoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate

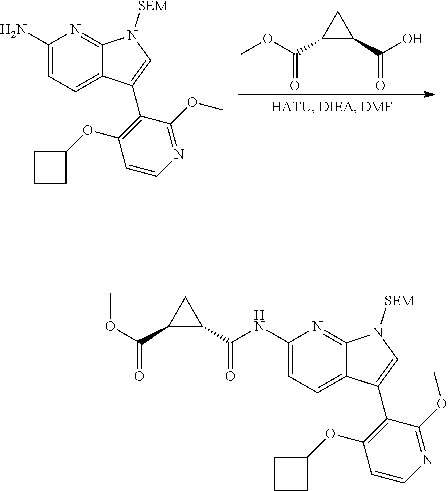

To a solution of 3-(4-cyclobutoxy-2-methoxypyridin-3-yl)-1-{[2-(trimethylsilyl)ethoxy]methyl}pyrrolo[2,3-b]pyridin-6-amine (1.7 g, 3.86 mmol) in DMF (40.0 mL) was added DIEA (2.5 g, 19.29 mmol), trans-2-(methoxycarbonyl)cyclopropane-1-carboxylic acid (0.7 g, 4.63 mmol) and HATU (1.8 g, 4.63 mmol) at 0° C. The resulting mixture was stirred at room temperature for 3 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (7/1, v/v) to afford methyl trans-2-{[3-(4-cyclobutoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate (853.0 mg, 46%) as a yellow oil. LCMS (ESI, m/z): [M+H]⁺=567.3.

### Step 8: Synthesis of Trans-N-[3-(4-cyclobutoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide

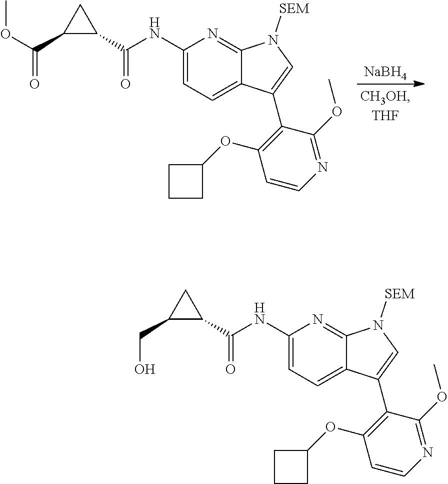

To a solution of methyl trans-2-{[3-(4-cyclobutoxy-2-methoxypyridin-3-yl)-1-{[2-(trimethylsilyl)ethoxy]methyl}pyrrolo[2,3-b]pyridin-6-yl]carbamoyl}cyclopropane-1-carboxylate (800.0 mg, 1.41 mmol) in CH₃OH/THF (6.0 mL/9.0 mL) was added NaBH₄ (534.1 mg, 14.12 mmol) at room temperature. The resulting mixture was stirred at room temperature for 3 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with CH₃CN/H₂O (6/1, v/v) to afford trans-N-[3-(4-cyclobutoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (651.4 mg, 86%) as a yellow oil. LCMS (ESI, m/z): [M+H]⁺=539.3.

### Step 9: Synthesis of Trans-N-[3-(4-cyclobutoxy-2-methoxypyridin-3-yl)-1-{[2-(trimethylsilyl)ethoxy]methyl}pyrrolo[2,3-b]pyridin-6-yl]-2-formylcyclopropane-1-carboxamide

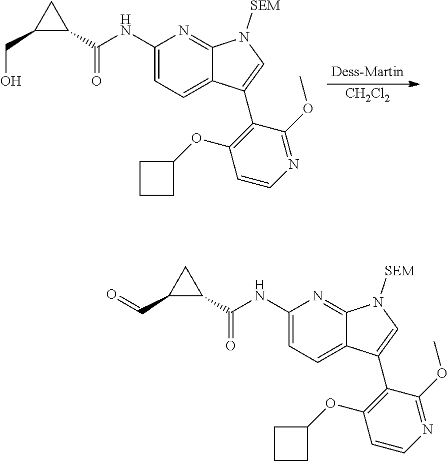

To a solution of trans-N-[3-(4-cyclobutoxy-2-methoxypyridin-3-yl)-1-{[2-(trimethylsilyl)ethoxy]methyl}pyrrolo[2,3-b]pyridin-6-yl]-2-(hydroxymethyl)cyclopropane-1-carboxamide (600.0 mg, 1.11 mmol) in CH₂Cl₂ (18.0 mL) was added Dess-Martin (708.6 mg, 1.67 mmol) at room temperature. The resulting mixture was stirred at room temperature for 3 h. After the reaction was completed, The reaction was diluted with aq·NaHCO₃ and extracted with CH₂Cl₂. The combined organic layer was washed with brine, dried over anhydrous sodium sulfate and filtered. The filtrate was concentrated under reduced pressure to afford trans-N-[3-(4-cyclobutoxy-2-methoxypyridin-3-yl)-1-{[2-(trimethylsilyl)ethoxy]methyl}pyrrolo[2,3-b]pyridin-6-yl]-2-formylcyclopropane-1-carboxamide (800.0 mg, crude) as a white oil. LCMS (ESI, m/z): [M+H]⁺=537.2.

### Step 10: Synthesis of Trans-N-[3-(4-cyclobutoxy-2-methoxypyridin-3-yl)-1-{[2-(trimethylsilyl)ethoxy]methyl}pyrrolo[2,3-b]pyridin-6-yl]-2-[(dimethylamino)methyl]cyclopropane-1-carboxamide

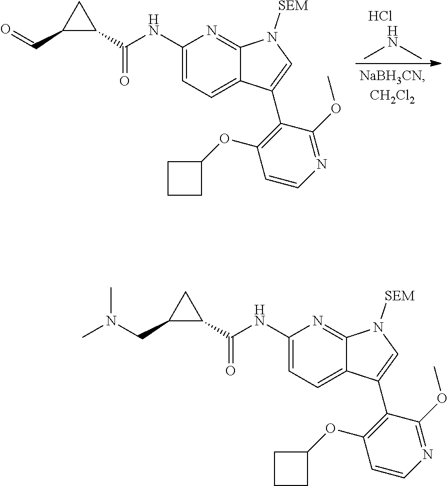

To a solution of trans-N-[3-(4-cyclobutoxy-2-methoxypyridin-3-yl)-1-{[2-(trimethylsilyl)ethoxy]methyl}pyrrolo[2,3-b]pyridin-6-yl]-2-formylcyclopropane-1-carboxamide (480.0 mg, 0.89 mmol) in CH₂Cl₂ (15.0 mL) was added dimethylamine hydrochloride (218.8 mg, 2.68 mmol) and NaBH₃CN (168.6 mg, 2.68 mmol) at room temperature. The resulting mixture was stirred at room temperature for 1 h. After the reaction was completed, the resulting mixture was concentrated under reduced pressure. The residue was purified by flash column chromatography with CH₂Cl₂/MeOH (6/1, v/v) to afford trans-N-[3-(4-cyclobutoxy-2-methoxypyridin-3-yl)-1-{[2-(trimethylsilyl)ethoxy]methyl}pyrrolo[2,3-b]pyridin-6-yl]-2-[(dimethylamino)methyl]cyclopropane-1-carboxamide (400.0 mg, 79%) as a yellow oil. LCMS (ESI, m/z): [M+H]⁺=566.3.

### Step 11: Synthesis of Trans-N-[3-(4-cyclobutoxy-2-methoxypyridin-3-yl)-1H-pyrrolo[2,3-b]pyridin-6-yl]-2-[(dimethylamino)methyl]cyclopropane-1-carboxamide

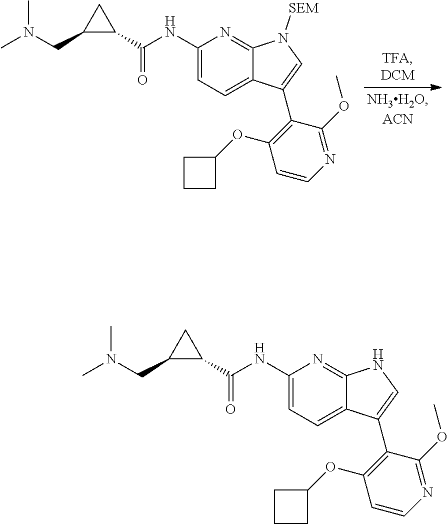 [SMILES: COc1nccc(OC2CCC2)c1-c1cn(COCC[Si](C)(C)C)c2nc(NC(=O)[C@H]3C[C@@H]3CN(C)C)ccc12 ; COc1nccc(OC2CCC2)c1-c1c[nH]c2nc(NC(=O)[C@H]3C[C@@H]3CN(C)C)ccc12]

To a solution of trans-N-[3-(4-cyclobutoxy-2-methoxypyridin-3-yl)-1-{[2-(trimethylsilyl)ethoxy]methyl}pyrrolo[2,3-b]pyridin-6-yl]-2-[(dimethylamino)methyl]cyclopropane-1-carboxamide (400.0 mg, 0.71 mmol) in DCM (6.0 mL) was added TFA (6.0 mL) at room temperature. The resulting mixture was stirred at room temperature for 1 h. The mixture was evaporated under reduced pressure. To the above mixture was added ACN/NH₃·H₂O (6.0 mL/6.0 mL) at room temperature. The resulting mixture was stirred at room temperature for additional 1 h. After the reaction was completed, the resulting mixture was concentrated under reduced pressure. The residue was purified by reverse phase flash column chromatography with CH₃CN/H₂O (3/7, v/v) to afford trans-N-[3-(4-cyclobutoxy-2-methoxypyridin-3-yl)-1H-pyrrolo[2,3-b]pyridin-6-yl]-2-[(dimethylamino)methyl]cyclopropane-1-carboxamide (75.0 mg, 24%) as a yellow oil. LCMS (ESI, m/z): [M+H]⁺=436.2.

### Step 12: Synthesis of (1S,2S)—N-[3-(4-cyclobutoxy-2-methoxypyridin-3-yl)-1H-pyrrolo[2,3-b]pyridin-6-yl]-2-[(dimethylamino)methyl]cyclopropane-1-carboxamide and (1R,2R)—N-[3-(4-cyclobutoxy-2-methoxypyridin-3-yl)-1H-pyrrolo[2,3-b]pyridin-6-yl]-2-[(dimethylamino)methyl]cyclopropane-1-carboxamide (Compound 242 and Compound 243)

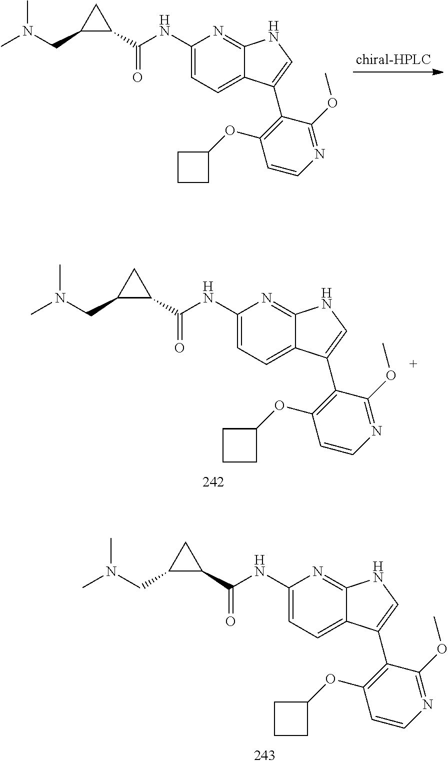 [SMILES: COc1nccc(OC2CCC2)c1-c1c[nH]c2nc(NC(=O)[C@H]3C[C@@H]3CN(C)C)ccc12 ; COc1nccc(OC2CCC2)c1-c1c[nH]c2nc(NC(=O)[C@H]3C[C@@H]3CN(C)C)ccc12 ; COc1nccc(OC2CCC2)c1-c1c[nH]c2nc(NC(=O)[C@@H]3C[C@H]3CN(C)C)ccc12]

The product trans-N-[3-(4-cyclobutoxy-2-methoxypyridin-3-yl)-1H-pyrrolo[2,3-b]pyridin-6-yl]-2-[(dimethylamino)methyl]cyclopropane-1-carboxamide (75.0 mg, 0.17 mmol) was separated by Prep-Chiral-HPLC with the following conditions (Column: CHIRAL ART Cellulose-SC, 2×25 cm, 5 m; Mobile Phase A:Hex:DCM=3:1 (0.5% 2M NH₃-MeOH)—HPLC, Mobile Phase B: EtOH—HPLC; Flow rate: 20 mL/min; Gradient: 15% B to 15% B in 8 min; Wave Length: 220/254 nm) to afford N-[3-(4-cyclobutoxy-2-methoxypyridin-3-yl)-1H-pyrrolo[2,3-b]pyridin-6-yl]-2-[(dimethylamino)methyl]cyclopropane-1-carboxamide Enantiomer 1 (7.2 mg, 19%) as a white solid and N-[3-(4-cyclobutoxy-2-methoxypyridin-3-yl)-1H-pyrrolo[2,3-b]pyridin-6-yl]-2-[(dimethylamino)methyl]cyclopropane-1-carboxamide Enantiomer 2 (5.8 mg, 15%) as a white solid. The absolute stereochemistry of Enantiomers 1 and 2 was not assigned. The two enantiomeric structures that could be obtained from chiral separation of the enantiomeric mixture as described above are shown as Compounds 242 and 243 in Table 1.

N-[3-(4-cyclobutoxy-2-methoxypyridin-3-yl)-1H-pyrrolo[2,3-b]pyridin-6-yl]-2-[(dimethylamino)methyl]cyclopropane-1-carboxamide Enantiomer 1: Retention Time 1 (min): 5.14; LCMS (ESI, m/z): [M+H]⁺=436.2. ¹H NMR (400 MHz, DMSO-d₆): δ 11.52 (s, 1H), 10.53 (s, 1H), 8.00 (d, J=5.6 Hz, 1H), 7.86 (d, J=8.4 Hz, 1H), 7.66 (d, J=8.4 Hz, 1H), 7.41 (d, J=2.4 Hz, 1H), 6.68 (d, J=5.6 Hz, 1H), 4.80-4.76 (m, 1H), 3.81 (s, 3H), 2.43-2.37 (m, 2H), 2.33-2.28 (m, 1H), 2.20-2.15 (m, 6H), 1.98-1.91 (m, 4H), 1.77-1.74 (m, 1H), 1.65-1.60 (m, 1H), 1.37-1.28 (m, 1H), 1.09-1.01 (m, 1H), 0.72-0.68 (m, 1H).

N-[3-(4-cyclobutoxy-2-methoxypyridin-3-yl)-1H-pyrrolo[2,3-b]pyridin-6-yl]-2-[(dimethylamino)methyl]cyclopropane-1-carboxamide Enantiomer 2: Retention Time 2 (min): 6.57; LCMS (ESI, m/z): [M+H]⁺=436.2. ¹H NMR (400 MHz, DMSO-d₆): δ 11.53 (s, 1H), 10.55 (s, 1H), 7.99 (d, J=6.0 Hz, 1H), 7.86 (d, J=8.8 Hz, 1H), 7.66 (d, J=8.8 Hz, 1H), 7.41 (d, J=2.4 Hz, 1H), 6.68 (d, J=5.6 Hz, 1H), 4.79-4.76 (m, 1H), 3.81 (s, 3H), 2.43-2.37 (m, 2H), 2.32-2.27 (m, 1H), 2.20-2.15 (m, 6H), 1.98-1.89 (m, 4H), 1.76-1.74 (m, 1H), 1.65-1.60 (m, 1H), 1.36-1.24 (m, 1H), 1.07-1.02 (m, 1H), 0.73-0.68 (m, 1H).

### Example S216: Synthesis of (1S,2R)—N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide and (1R,2S)—N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide (Compound 244 and Compound 245)

### Step 1: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide

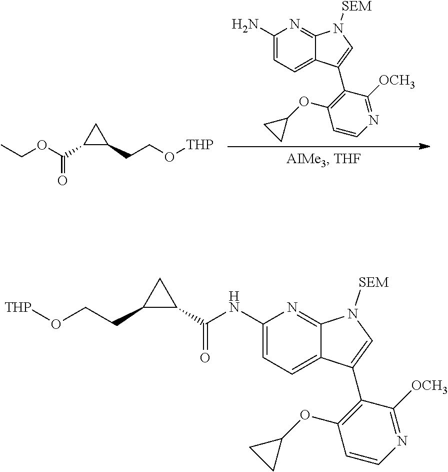

To a mixture of ethyl trans-2-(2-((tetrahydro-2H-pyran-2-yl)oxy)ethyl)cyclopropane-1-carboxylate (0.9 g, 3.52 mmol) and 3-(4-cyclopropoxy-2-methoxypyridin-3-yl)-1-((2-(trimethylsilyl)ethoxy)methyl)-1H-pyrrolo[2,3-b]pyridin-6-amine (1.0 g, 2.34 mmol) in THF (40.0 mL) was added AlMe₃ (3.5 mL, 2 mol/L) at 0° C. under N₂. The resulting mixture was stirred at 80° C. for 2 h. After the reaction was completed, the resulting mixture was quenched with H₂O. The mixture was concentrated under reduced pressure. The residue was purified by flash column chromatography with CH₂Cl₂/CH₃OH (10/1, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide (1.6 g, 87%) as a yellow solid. LCMS (ESI, m/z): [M+H]⁺=623.3.

### Step 2: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-hydroxyethyl)cyclopropane-1-carboxamide

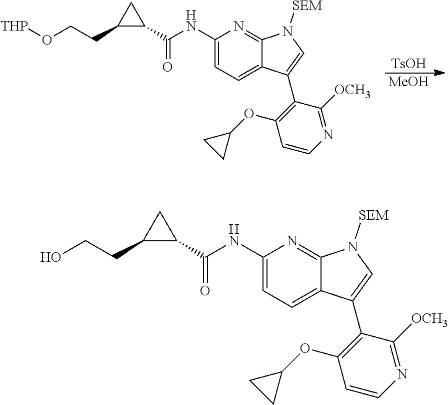

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-((tetrahydro-2H-pyran-2-yl)oxy)ethyl)cyclopropane-1-carboxamide (1.6 g, 2.57 mmol) in MeOH (30.0 mL) was added TsOH (0.4 g, 2.57 mmol) at room temperature. The resulting mixture was stirred at room temperature for 4 h. After the reaction was completed, the mixture was concentrated under reduced pressure. The residue was purified by flash column chromatography with CH₂Cl₂/CH₃OH (5/1, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-hydroxyethyl)cyclopropane-1-carboxamide (450.0 mg, 32%) as a brown solid. LCMS (ESI, m/z): [M+H]⁺=539.3.

### Step 3: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-oxoethyl)cyclopropane-1-carboxamide

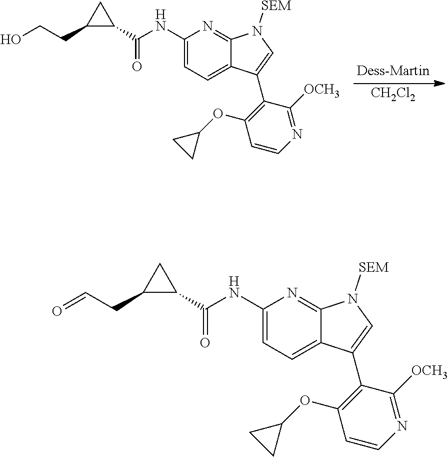

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-hydroxyethyl)cyclopropane-1-carboxamide (400.0 mg, 0.74 mmol) in CH₂Cl₂ (30.0 mL) was added Dess-Martin (472.4 mg, 1.12 mmol) at 0° C. The resulting mixture was stirred at room temperature for 1 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-oxoethyl)cyclopropane-1-carboxamide (400.0 mg, crude) as a yellow solid. LCMS (ESI, m/z): [M+H]⁺=537.3.

### Step 4: Synthesis of Trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethyl)cyclopropane-1-carboxamide

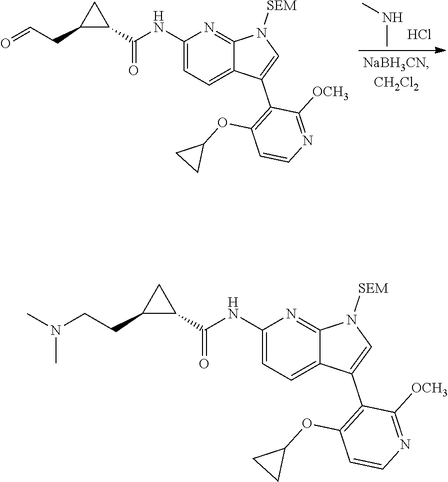

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-oxoethyl)cyclopropane-1-carboxamide (400.0 mg, crude) in CH₂Cl₂ (20.0 mL) was added dimethylamine hydrochloride (182.3 mg, 2.24 mmol) and NaBH₃CN (140.5 mg, 2.24 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with CH₂Cl₂/CH₃OH (5/1, v/v) to afford trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethyl)cyclopropane-1-carboxamide (90.0 mg, 21%) as a yellow solid. LCMS (ESI, m/z): [M+H]⁺=566.3.

### Step 5: Synthesis of Trans-N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide

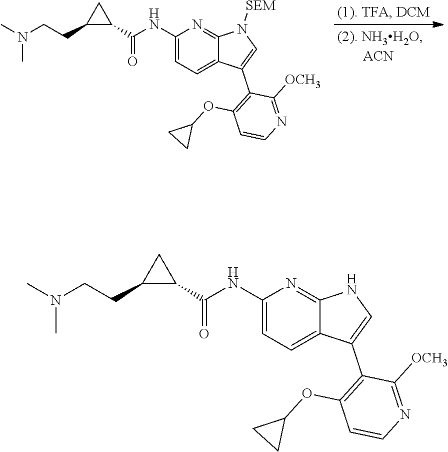 [SMILES: COc1nccc(OC2CC2)c1-c1cn(COCC[Si](C)(C)C)c2nc(NC(=O)[C@H]3C[C@@H]3CCN(C)C)ccc12 ; COc1nccc(OC2CC2)c1-c1c[nH]c2nc(NC(=O)[C@H]3C[C@@H]3CCN(C)C)ccc12]

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethyl)cyclopropane-1-carboxamide (90.0 mg, 0.16 mmol) in CH₂Cl₂ (2.0 mL) was added TFA (2.0 mL) at room temperature. The resulting mixture was stirred at room temperature for 3 h. The resulting mixture was concentrated under reduced pressure. To the residue in CH₃CN (2.0 mL) was added NH₃·H₂O (2.0 mL) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (5/1, v/v) to afford trans-N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide (50.0 mg, 72%) as a white solid. LCMS (ESI, m/z): [M+H]⁺=436.2.

### Step 6: Synthesis of (1S,2R)—N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide and (1R,2S)—N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide (Compound 244 and Compound 245)

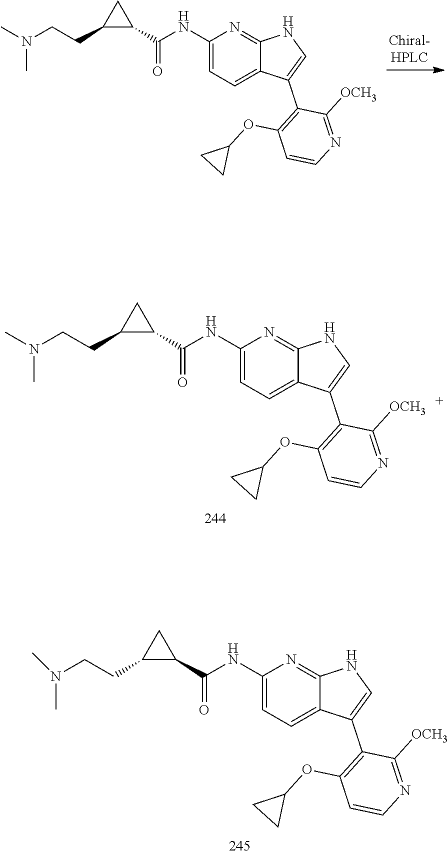 [SMILES: COc1nccc(OC2CC2)c1-c1c[nH]c2nc(NC(=O)[C@H]3C[C@@H]3CCN(C)C)ccc12 ; COc1nccc(OC2CC2)c1-c1c[nH]c2nc(NC(=O)[C@H]3C[C@@H]3CCN(C)C)ccc12 ; COc1nccc(OC2CC2)c1-c1c[nH]c2nc(NC(=O)[C@@H]3C[C@H]3CCN(C)C)ccc12]

The product of trans-N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide (50.0 mg, 0.11 mmol) was separated by Prep-Chiral-HPLC with the following conditions (Column CHIRALPAK IE, 2×25 cm, 5 m; Mobile Phase A: Hex (0.5% 2M NH₃-MeOH)—HPLC, Mobile Phase B: EtOH—HPLC; Flow rate: 12 mL/min; Gradient: 50% to 50% in 27 min; Wave Length: 220/254 nm) to afford N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide Enantiomer 1 (5.3 mg, 10%) as a white solid and N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide Enantiomer 2 (5.1 mg, 10%) as a white solid. The absolute stereochemistry of Enantiomers 1 and 2 was not assigned. The two enantiomeric structures that could be obtained from chiral separation of the enantiomeric mixture as described above are shown as Compounds 244 and 245 in Table 1.

N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide Enantiomer 1: Retention Time 1 (min): 12.79; LCMS (ESI, m/z): [M+H]⁺=436.3. ¹H NMR (400 MHz, DMSO-d₆): δ 11.49 (s, 1H), 10.46 (s, 1H), 8.07 (d, J=6.0 Hz, 1H), 7.83 (d, J=8.4 Hz, 1H), 7.55 (d, J=8.8 Hz, 1H), 7.34 (s, 1H), 7.13 (d, J=5.6 Hz, 1H), 4.01-3.93 (m, 1H), 3.81 (s, 3H), 2.33-2.30 (m, 2H), 2.13 (s, 6H), 1.88-1.82 (m, 1H), 1.47-1.37 (m, 2H), 1.24-1.16 (m, 1H), 1.09-0.91 (m, 1H), 0.89-0.75 (m, 2H), 0.73-0.58 (m, 3H).

N-(3-(4-cyclopropoxy-2-methoxypyridin-3-yl)-1H-pyrrolo[2,3-b]pyridin-6-yl)-2-(2-(dimethylamino)ethyl)cyclopropane-1-carboxamide Enantiomer 2: Retention Time 2 (min): 18.32; LCMS (ESI, m/z): [M+H]⁺=436.3. ¹H NMR (400 MHz, DMSO-d₆): δ 11.49 (s, 1H), 10.46 (s, 1H), 8.07 (d, J=5.6 Hz, 1H), 7.83 (d, J=8.4 Hz, 1H), 7.55 (d, J=8.8 Hz, 1H), 7.34 (s, 1H), 7.14 (d, J=5.6 Hz, 1H), 3.99-3.93 (m, 1H), 3.81 (s, 3H), 2.34-2.31 (m, 2H), 2.13 (s, 6H), 1.89-1.84 (m, 1H), 1.47-1.35 (m, 2H), 1.24-1.16 (m, 1H), 1.12-1.00 (m, 1H), 0.86-0.73 (m, 2H), 0.68-0.64 (m, 3H).

### Example S217. Synthesis of (1R)—N-[3-(2,6-dimethoxyphenyl)-1H-pyrrolo[2,3-b]pyridin-6-yl]-5-methyl-5-azaspiro[2.3]hexane-1-carboxamide and (1S)—N-[3-(2,6-dimethoxyphenyl)-1H-pyrrolo[2,3-b]pyridin-6-yl]-5-methyl-5-azaspiro[2.3]hexane-1-carboxamide (Compound 246 and Compound 247)

### Step 1: Synthesis of tert-butyl 1-{[3-(2,6-dimethoxyphenyl)-1-{[2-(trimethylsilyl)ethoxy]methyl}pyrrolo[2,3-b]pyridin-6-yl]carbamoyl}-5-azaspiro[2.3]hexane-5-carboxylate

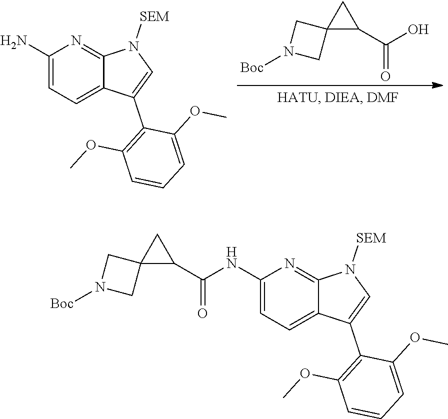 [SMILES: COc1cccc(OC)c1-c1cn(COCC[Si](C)(C)C)c2nc(N)ccc12 ; CC(C)(C)OC(=O)N1CC2(CC2C(=O)O)C1 ; COc1cccc(OC)c1-c1cn(COCC[Si](C)(C)C)c2nc(NC(=O)C3CC34CN(C(=O)OC(C)(C)C)C4)ccc12]

To a solution of 3-(2,6-dimethoxyphenyl)-1-{[2-(trimethylsilyl)ethoxy]methyl}pyrrolo[2,3-b]pyridin-6-amine (1.8 g, 4.41 mmol) in DMF (30.0 mL) were added 5-(tert-butoxycarbonyl)-5-azaspiro[2.3]hexane-1-carboxylic acid (1.0 g, 4.41 mmol), DIEA (2.3 g, 17.64 mmol) and HATU (2.4 g, 6.17 mmol) at room temperature. The resulting mixture was stirred at room temperature for 1 h. After the reaction was completed, the resulting mixture was diluted with water and extracted with ethyl acetate. The combined organic layers were washed with brine, dried over anhydrous sodium sulfate and filtered. The filtrate was concentrated under reduced pressure. The residue was purified by flash column chromatography with petroleum ether/ethyl acetate (3/1, v/v) to afford tert-butyl 1-{[3-(2,6-dimethoxyphenyl)-1-{[2-(trimethylsilyl)ethoxy]methyl}pyrrolo[2,3-b]pyridin-6-yl]carbamoyl}-5-azaspiro[2.3]hexane-5-carboxylate (2.2 g, 83%) as a yellow solid. LCMS (ESI, m/z): [M+H]⁺=609.3.

### Step 2: Synthesis of N-[3-(2,6-dimethoxyphenyl)-1-{[2-(trimethylsilyl)ethoxy]methyl}pyrrolo[2,3-b]pyridin-6-yl]-5-azaspiro[2.3]hexane-1-carboxamide

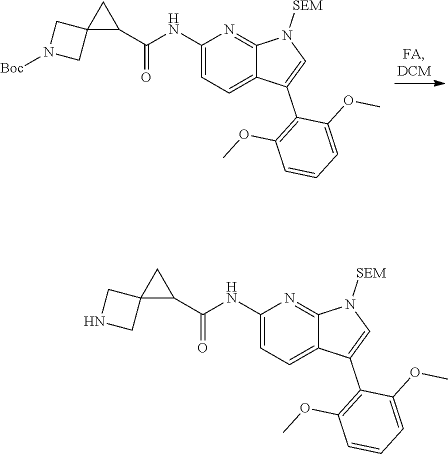 [SMILES: COc1cccc(OC)c1-c1cn(COCC[Si](C)(C)C)c2nc(NC(=O)C3CC34CN(C(=O)OC(C)(C)C)C4)ccc12 ; COc1cccc(OC)c1-c1cn(COCC[Si](C)(C)C)c2nc(NC(=O)C3CC34CNC4)ccc12]

To a solution of tert-butyl 1-{[3-(2,6-dimethoxyphenyl)-1-{[2-(trimethylsilyl)ethoxy]methyl}pyrrolo[2,3-b]pyridin-6-yl]carbamoyl}-5-azaspiro[2.3]hexane-5-carboxylate (1.0 g, 1.64 mmol) in DCM (10.0 mL) was added HCOOH (20.0 mL) at room temperature. The resulting mixture was stirred at room temperature for 16 h. After the reaction was completed, the resulting mixture was diluted with water. The pH value of the mixture was adjusted to 8 with aq·NaHCO₃. The resulting mixture was extracted with DCM. The combined organic layer was washed with brine, dried over anhydrous sodium sulfate and filtered. The filtrate was concentrated under reduced pressure. The residue was purified by flash column chromatography with CH₂Cl₂/MeOH (4/1, v/v) to afford N-[3-(2,6-dimethoxyphenyl)-1-{[2-(trimethylsilyl)ethoxy]methyl}pyrrolo[2,3-b]pyridin-6-yl]-5-azaspiro[2.3]hexane-1-carboxamide (0.4 g, 45%) as a yellow solid. LCMS (ESI, m/z): [M+H]⁺=509.3.

### Step 3: Synthesis of N-[3-(2,6-dimethoxyphenyl)-1-{[2-(trimethylsilyl)ethoxy]methyl}pyrrolo[2,3-b]pyridin-6-yl]-5-methyl-5-azaspiro[2.3]hexane-1-carboxamide

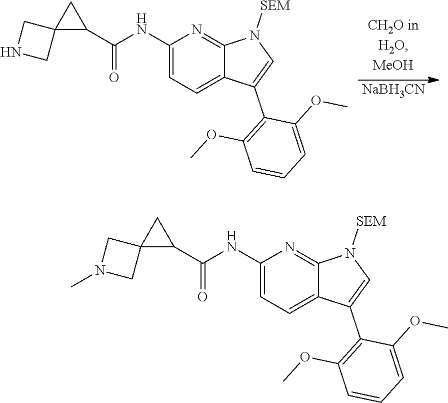

To a solution of N-[3-(2,6-dimethoxyphenyl)-1-{[2-(trimethylsilyl)ethoxy]methyl}pyrrolo[2,3-b]pyridin-6-yl]-5-azaspiro[2.3]hexane-1-carboxamide (550.0 mg, 1.08 mmol) in CH₃OH (20.0 mL) was added a solution of HCHO in H₂O (1.1 mL, 37%) at room temperature. The resulting mixture was stirred at room temperature for 10 min. Then NaBH₃CN (203.8 mg, 3.24 mmol) was added to the mixture. The resulting mixture was stirred at room temperature for 30 min. After the reaction was completed, the resulting mixture was concentrated under reduced pressure. The residue was purified by reverse phase flash chromatography with acetonitrile/water (2/3, v/v) to afford N-[3-(2,6-dimethoxyphenyl)-1-{[2-(trimethylsilyl)ethoxy]methyl}pyrrolo[2,3-b]pyridin-6-yl]-5-methyl-5-azaspiro[2.3]hexane-1-carboxamide (130.0 mg, 23%) as a white solid. LCMS (ESI, m/z): [M+H]⁺=523.3.

### Step 4: Synthesis of N-(3-(2,6-dimethoxyphenyl)-1H-pyrrolo[2,3-b]pyridin-6-yl)-5-methyl-5-azaspiro[2.3]hexane-1-carboxamide

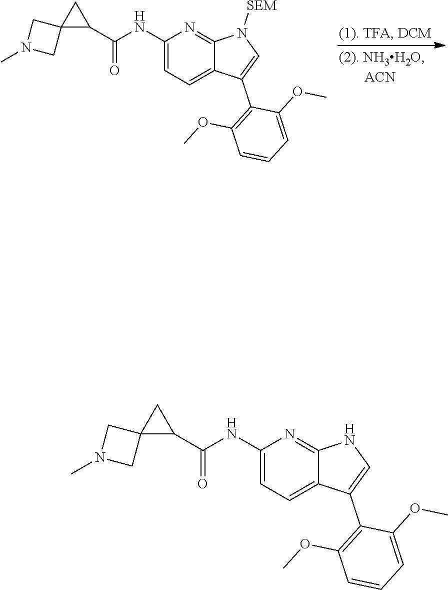

To a solution of N-[3-(2,6-dimethoxyphenyl)-1-{[2-(trimethylsilyl)ethoxy]methyl}pyrrolo[2,3-b]pyridin-6-yl]-5-methyl-5-azaspiro[2.3]hexane-1-carboxamide (176.0 mg, 0.34 mmol) in DCM (10.0 mL) was added TFA (10.0 mL) at room temperature. The resulting mixture was stirred at room temperature for 1.5 h. The resulting mixture was concentrated under reduced pressure. To the above mixture was added CH₃CN (6.0 mL) and NH₃·H₂O (6.0 mL) at room temperature. The resulting mixture was stirred at room temperature for 1 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash chromatography with CH₃CN/H₂O (2/3, v/v) to afford N-(3-(2,6-dimethoxyphenyl)-1H-pyrrolo[2,3-b]pyridin-6-yl)-5-methyl-5-azaspiro[2.3]hexane-1-carboxamide (130.0 mg, 98%) as a white solid. LCMS (ESI, m/z): [M+H]⁺=393.2.

### Step 5: Synthesis of (1R)—N-[3-(2,6-dimethoxyphenyl)-1H-pyrrolo[2,3-b]pyridin-6-yl]-5-methyl-5-azaspiro[2.3]hexane-1-carboxamide and (1S)—N-[3-(2,6-dimethoxyphenyl)-1H-pyrrolo[2,3-b]pyridin-6-yl]-5-methyl-5-azaspiro[2.3]hexane-1-carboxamide (Compound 246 and Compound 247)

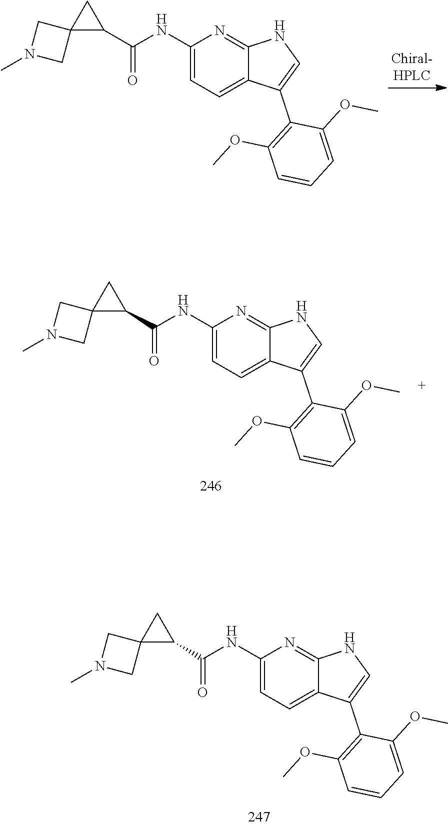

The racemic N-(3-(2,6-dimethoxyphenyl)-1H-pyrrolo[2,3-b]pyridin-6-yl)-5-methyl-5-azaspiro[2.3]hexane-1-carboxamide (130.0 mg, 0.33 mmol) was separated by Chiral-Prep-HPLC with the following conditions (Column: CHIRALPAK IG, 2×25 cm, 5 m; Mobile Phase A: Hex: DCM=1: 1 (0.5% 2M NH₃-MeOH)—HPLC, Mobile Phase B: EtOH—HPLC; Flow rate: 17 mL/min; Gradient: 50% B to 50% B in 14.5 min; Wave Length: 220/254 nm) to afford N-[3-(2,6-dimethoxyphenyl)-1H-pyrrolo[2,3-b]pyridin-6-yl]-5-methyl-5-azaspiro[2.3]hexane-1-carboxamide Enantiomer 1 (13.5 mg, 13%) as a yellow solid and N-[3-(2,6-dimethoxyphenyl)-1H-pyrrolo[2,3-b]pyridin-6-yl]-5-methyl-5-azaspiro[2.3]hexane-1-carboxamide Enantiomer 2 (14.0 mg, 12%) as a white solid. The absolute stereochemistry of Enantiomers 1 and 2 was not assigned. The two enantiomeric structures that could be obtained from chiral separation of the enantiomeric mixture as described above are shown as Compounds 246 and 247 in Table 1.

N-[3-(2,6-dimethoxyphenyl)-1H-pyrrolo[2,3-b]pyridin-6-yl]-5-methyl-5-azaspiro[2.3]hexane-1-carboxamide Enantiomer 1: Retention Time 1: 4.65 min; LCMS (ESI, m/z): [M+H]⁺=393.2. ¹H NMR (400 MHz, DMSO-d₆): δ 11.41 (s, 1H), 10.50 (s, 1H), 7.80 (d, J=8.4 Hz, 1H), 7.52 (d, J=8.4 Hz, 1H), 7.30-7.27 (m, 2H), 6.76-6.72 (m, 2H), 3.67 (s, 6H), 3.36-3.24 (m, 4H), 2.30 (s, 3H), 2.13-2.10 (m, 1H), 1.10-1.04 (m, 2H).

N-[3-(2,6-dimethoxyphenyl)-1H-pyrrolo[2,3-b]pyridin-6-yl]-5-methyl-5-azaspiro[2.3]hexane-1-carboxamide Enantiomer 2: Retention Time 2: 10.92 min; LCMS (ESI, m/z): [M+H]⁺=393.2. ¹H NMR (400 MHz, DMSO-d₆): δ 11.41 (s, 1H), 10.49 (s, 1H), 7.80 (d, J=8.4 Hz, 1H), 7.53 (d, J=8.4 Hz, 1H), 7.30-7.22 (m, 2H), 6.76-6.70 (m, 2H), 3.72 (s, 6H), 3.34-3.21 (m, 4H), 2.29 (s, 3H), 2.15-2.02 (m, 1H), 1.12-1.03 (m, 2H).

### Example S218: Synthesis of (1R,2R)-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide and (1S,2S)-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide (Compound 248 and Compound 249)

### Step 1: Synthesis of Trans-2-((bis(methyl-d₃)amino)methyl)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide

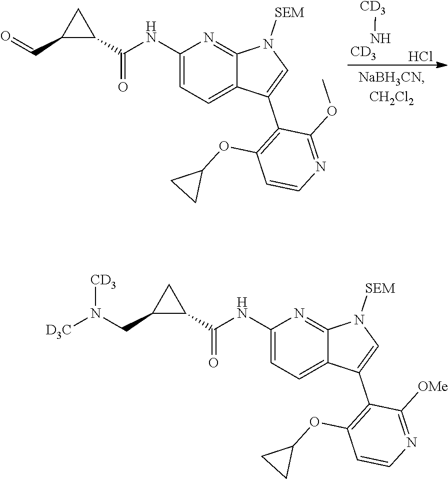

To a solution of trans-N-(3-(4-cyclopropoxy-2-methoxypyridin-3-yl)-1-((2-(trimethylsilyl)ethoxy)methyl)-1H-pyrrolo[2,3-b]pyridin-6-yl)-2-formylcyclopropane-1-carboxamide (670.0 mg, 1.28 mmol) in CH₂Cl₂ (20.0 mL) was added dimethyl-D6-amine hydrochloride (336.8 mg, 3.85 mmol) and NaBH₃CN (241.7 mg, 3.87 mmol) at room temperature. The resulting mixture was stirred at room temperature for 1 h. After the reaction was completed, the reaction was quenched with H₂O and extracted with dichloromethane. The combined organic layer was washed with brine, dried over anhydrous sodium sulfate and filtered. The filtrate was concentrated under reduced pressure. The residue was purified by reverse phase flash column chromatography with H₂O/CH₃CN (3/7, v/v) to afford trans-2-((bis(methyl-d₃)amino)methyl)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide (200.0 mg, 27%) as a green solid. LCMS (ESI, m/z): [M+H]⁺=558.3.

### Step 2: Synthesis of Trans-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide

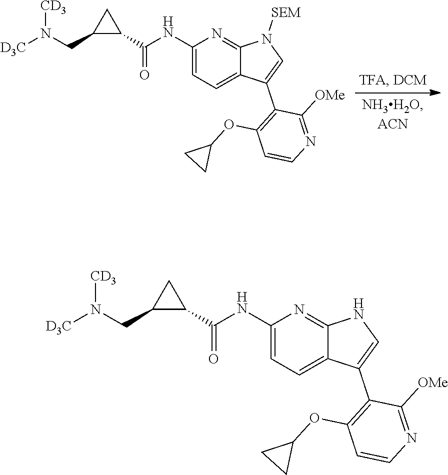 [SMILES: [2H]C([2H])([2H])N(C[C@H]1C[C@@H]1C(=O)Nc1ccc2c(-c3c(OC4CC4)ccnc3OC)cn(COCC[Si](C)(C)C)c2n1)C([2H])([2H])[2H] ; [2H]C([2H])([2H])N(C[C@H]1C[C@@H]1C(=O)Nc1ccc2c(-c3c(OC4CC4)ccnc3OC)c[nH]c2n1)C([2H])([2H])[2H]]

To a solution of trans-2-((bis(methyl-d₃)amino)methyl)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide (300.0 mg, 0.54 mmol) in CH₂Cl₂ (5.0 mL) was added TFA (5.0 mL) at room temperature. The resulting mixture was stirred at room temperature for 2 h. The resulting mixture was concentrated under reduced pressure. To the residue in CH₃CN (5.0 mL) was added NH₃·H₂O (5.0 mL) at room temperature. The resulting mixture was stirred at room temperature for another 2 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/CH₃CN (1/1, v/v) to afford trans-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide (190.0 mg, 82%) as a white solid. LCMS (ESI, m/z): [M+H]⁺=428.3.

### Step 3: Synthesis of (1R,2R)-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide and (1S,2S)-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide (Compound 248 and Compound 249)

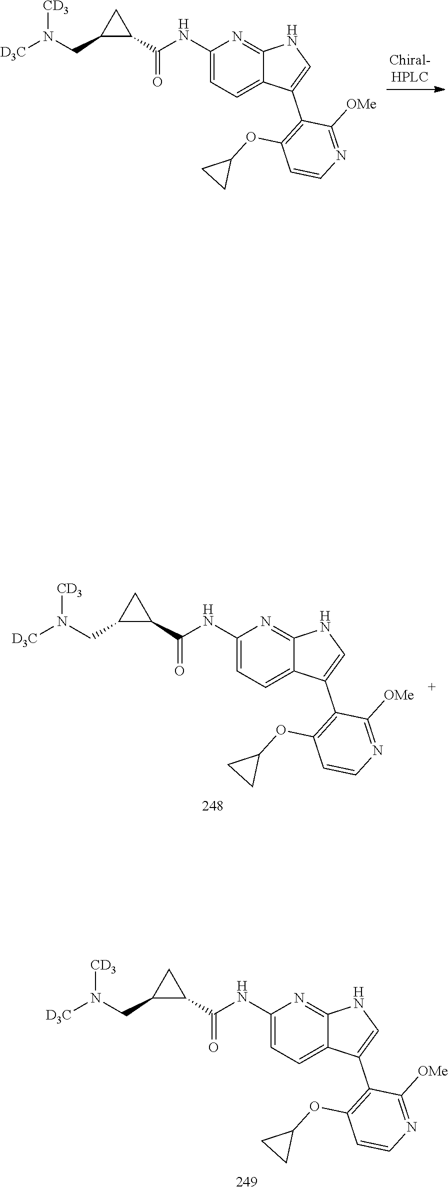 [SMILES: [2H]C([2H])([2H])N(C[C@H]1C[C@@H]1C(=O)Nc1ccc2c(-c3c(OC4CC4)ccnc3OC)c[nH]c2n1)C([2H])([2H])[2H] ; [2H]C([2H])([2H])N(C[C@@H]1C[C@H]1C(=O)Nc1ccc2c(-c3c(OC4CC4)ccnc3OC)c[nH]c2n1)C([2H])([2H])[2H] ; [2H]C([2H])([2H])N(C[C@H]1C[C@@H]1C(=O)Nc1ccc2c(-c3c(OC4CC4)ccnc3OC)c[nH]c2n1)C([2H])([2H])[2H]]

The product trans-2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide (190.0 mg, 0.34 mmol) was separated by Prep-Chiral-HPLC with the following conditions (Column: CHIRAL ART Cellulose-SC, 2×25 cm, 5 m; Mobile Phase A: Hex (0.5% 2M NH₃-MeOH)—HPLC, Mobile Phase B: MeOH: EtOH=1: 1—HPLC; Flow rate: 20 mL/min; Gradient: 20% to 20% in 17 min; Wave Length: 220/254 nm) to afford 2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide Enantiomer 1 (44.7 mg, 60%) as a white solid and 2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide Enantiomer 2 (30.1 mg, 40%) as a white solid. The absolute stereochemistry of Enantiomers 1 and 2 was not assigned. The two enantiomeric structures that could be obtained from chiral separation of the enantiomeric mixture as described above are shown as Compounds 248 and 249 in Table 1.

2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide Enantiomer 1: Retention Time 1 (min): 11.48; LCMS (ESI, m/z): [M+H]⁺=428.3. ¹H NMR (400 MHz, DMSO-d₆): δ 11.50 (s, 1H), 10.52 (s, 1H), 8.07 (d, J=5.6 Hz, 1H), 7.83 (d, J=8.8 Hz, 1H), 7.56 (d, J=8.4 Hz, 1H), 7.34 (d, J=2.0 Hz, 1H), 7.14 (d, J=5.6 Hz, 1H), 3.94-3.91 (m, 1H), 3.82 (s, 3H), 2.32-2.28 (m, 1H), 2.21-2.16 (m, 1H), 1.91-1.89 (m, 1H), 1.38-1.30 (m, 1H), 1.07-1.03 (m, 1H), 0.87-0.76 (m, 2H), 0.73-0.64 (m, 3H).

2-((bis(methyl-d₃)amino)methyl)-N-(3-(4-cyclopropoxy-2-methoxypyridin-3-yl)-1H-pyrrolo[2,3-b]pyridin-6-yl)cyclopropane-1-carboxamide Enantiomer 2: Retention Time 2 (min): 15.63; LCMS (ESI, m/z): [M+H]⁺=428.4. ¹H NMR (400 MHz, DMSO-d₆): δ 11.50 (s, 1H), 10.52 (s, 1H), 8.07 (d, J=6.0 Hz, 1H), 7.83 (d, J=8.8 Hz, 1H), 7.55 (d, J=8.4 Hz, 1H), 7.34 (d, J=2.0 Hz, 1H), 7.14 (d, J=5.6 Hz, 1H), 3.99-3.91 (m, 1H), 3.81 (s, 3H), 2.29-2.25 (m, 1H), 2.18-2.13 (m, 1H), 1.90-1.88 (m, 1H), 1.37-1.32 (m, 1H), 1.08-1.02 (m, 1H), 0.81-0.76 (m, 2H), 0.71-0.63 (m, 3H).

### Example S219: Synthesis of N-[3-[4-methoxy-6-(methylamino)pyridin-3-yl]-1H-pyrrolo[2,3-b]pyridin-6-yl]cyclopropanecarboxamide (Compound 250)

### Step 1: Synthesis of tert-butyl N-(5-bromo-4-methoxypyridin-2-yl)-N-methylcarbamate

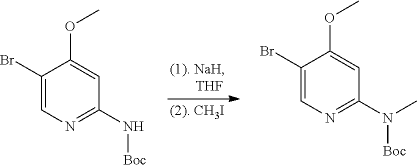 [SMILES: COc1cc(NC(=O)OC(C)(C)C)ncc1Br ; COc1cc(N(C)C(=O)OC(C)(C)C)ncc1Br]

To a solution of tert-butyl N-(5-bromo-4-methoxypyridin-2-yl)carbamate (635.0 mg, 2.10 mmol) in THF (20.0 mL) was added NaH (251.3 mg, 60%) at 0° C. under N₂. The resulting mixture was stirred at 0° C. for 1 h under N₂. Then methyl iodide (1.5 g, 10.47 mmol) was added dropwise to the mixture at 0° C. under N₂. The resulting mixture was stirred at 0° C. for 1 h. After the reaction was completed, the reaction mixture was quenched with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford tert-butyl N-(5-bromo-4-methoxypyridin-2-yl)-N-methylcarbamate (500.0 mg, 75%) as a yellow solid. LCMS (ESI, m/z): [M+H]⁺=317.0.

### Step 2: Synthesis of tert-butyl N-[5-(6-chloro-1-[[2-(trimethylsilyl)ethoxy]methyl]pyrrolo[2,3-b]pyridin-3-yl)-4-methoxypyridin-2-yl]-N-methylcarbamate

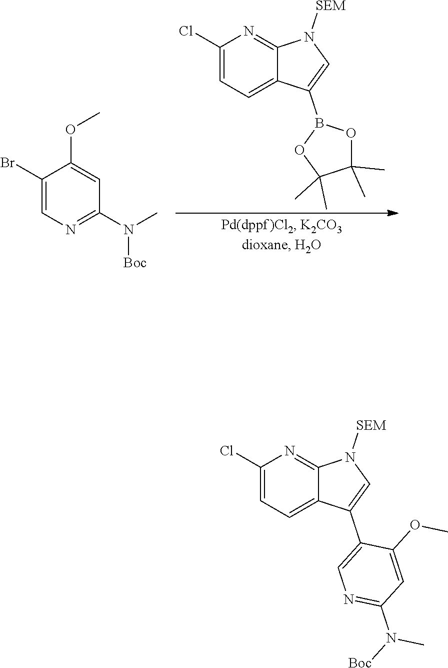 [SMILES: COc1cc(N(C)C(=O)OC(C)(C)C)ncc1Br ; CC1(C)OB(c2cn(COCC[Si](C)(C)C)c3nc(Cl)ccc23)OC1(C)C ; COc1cc(N(C)C(=O)OC(C)(C)C)ncc1-c1cn(COCC[Si](C)(C)C)c2nc(Cl)ccc12]

To a solution of tert-butyl N-(5-bromo-4-methoxypyridin-2-yl)-N-methylcarbamate (460.0 mg, 1.45 mmol) in dioxane/H₂O (10.0 mL/2.0 mL) was added 6-chloro-3-(4,4,5,5-tetramethyl-1,3,2-dioxaborolan-2-yl)-1-[[2-(trimethylsilyl)ethoxy]methyl]pyrrolo[2,3-b]pyridine (592.9 mg, 1.45 mmol), K₂CO₃ (601.3 mg, 4.35 mmol) and Pd(dppf)Cl₂ (118.4 mg, 0.15 mmol) at room temperature under N₂. The resulting mixture was stirred at 80° C. for 16 h. After the reaction was completed, the reaction was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford tert-butyl N-[5-(6-chloro-1-[[2-(trimethylsilyl)ethoxy]methyl]pyrrolo[2,3-b]pyridin-3-yl)-4-methoxypyridin-2-yl]-N-methylcarbamate (278.0 mg, 37%) as a colorless oil. LCMS (ESI, m/z): [M+H]⁺=519.2.

### Step 3: Synthesis of tert-butyl (5-(6-(cyclopropanecarboxamido)-1-((2-(trimethylsilyl)ethoxy)methyl)-1H-pyrrolo[2,3-b]pyridin-3-yl)-4-methoxypyridin-2-yl)(methyl)carbamate

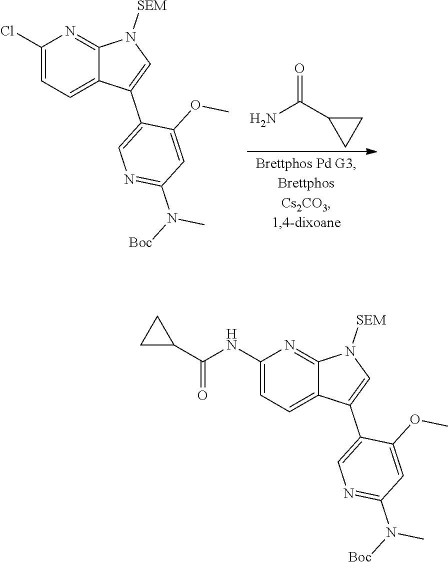

To a solution of tert-butyl N-[5-(6-chloro-1-[[2-(trimethylsilyl)ethoxy]methyl]pyrrolo[2,3-b]pyridin-3-yl)-4-methoxypyridin-2-yl]-N-methylcarbamate (258.0 mg, 0.50 mmol) in 1,4-dioxane (8.0 mL) was added cyclopropanecarboxamide (211.5 mg, 2.49 mmol), BrettPhos (53.4 mg, 0.10 mmol), Cs₂CO₃ (485.8 mg, 1.49 mmol) and BrettPhos Pd G3 (45.1 mg, 0.05 mmol) at room temperature under N₂. The resulting mixture was stirred with microwave at 120° C. for 1.5 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (2/1, v/v) to afford tert-butyl (5-(6-(cyclopropanecarboxamido)-1-((2-(trimethylsilyl)ethoxy)methyl)-1H-pyrrolo[2,3-b]pyridin-3-yl)-4-methoxypyridin-2-yl)(methyl)carbamate (168.0 mg, 72%) as a white oil. LCMS (ESI, m/z): [M+H]⁺=568.3.

### Step 4: Synthesis of N-[3-[4-methoxy-6-(methylamino)pyridin-3-yl]-1H-pyrrolo[2,3-b]pyridin-6-yl]cyclopropanecarboxamide (Compound 250)

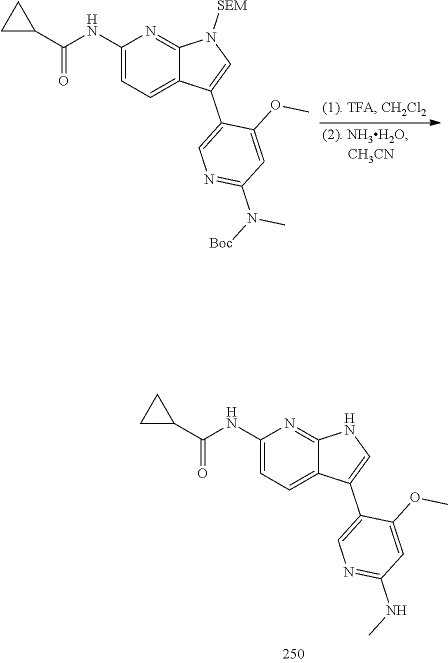 [SMILES: COc1cc(N(C)C(=O)OC(C)(C)C)ncc1-c1cn(COCC[Si](C)(C)C)c2nc(NC(=O)C3CC3)ccc12 ; CNc1cc(OC)c(-c2c[nH]c3nc(NC(=O)C4CC4)ccc23)cn1]

To a solution of tert-butyl N-[5-(6-cyclopropaneamido-1-[[2-(trimethylsilyl)ethoxy]methyl]pyrrolo[2,3-b]pyridin-3-yl)-4-methoxypyridin-2-yl]-N-methylcarbamate (168.0 mg, 0.30 mmol) in DCM (3.0 mL) was added TFA (3.0 mL) at room temperature. The resulting mixture was stirred at room temperature for 2 h. The resulting mixture was concentrated under reduced pressure. To the residue in CH₃CN (3.0 mL) was added NH₃·H₂O (3.0 mL) at room temperature. The resulting mixture was stirred at room temperature for 16 h. After the reaction was completed, the reaction mixture was diluted with H₂O and extracted with ethyl acetate. The combined organic layer was washed with brine, dried over anhydrous sodium sulfate and filtered. The filtrate was concentrated under reduced pressure. The residue was purified by Prep-HPLC with the following conditions Column: (XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 23% B to 53% B in 7 min; 254 nm) to afford N-[3-[4-methoxy-6-(methylamino)pyridin-3-yl]-1H-pyrrolo[2,3-b]pyridin-6-yl]cyclopropanecarboxamide (16.1 mg, 16%) as a white solid. LCMS (ESI, m/z): [M+H]⁺=338.2. ¹H NMR (300 MHz, DMSO-d₆): δ 11.39 (s, 1H), 10.52 (s, 1H), 8.00 (s, 1H), 7.91-7.82 (m, 2H), 7.36 (d, J=2.4 Hz, 1H), 6.38-6.34 (m, 1H), 6.10 (s, 1H), 3.78 (s, 3H), 2.81 (d, J=4.8 Hz, 3H), 2.07-1.99 (m, 1H), 0.82-0.74 (m, 4H).

### Example S220: Synthesis of (1R,2R)—N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide and (1S,2S)—N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide (Compound 251 and Compound 252)

### Step 1: Synthesis of tert-butyldiphenyl(2-(vinyloxy)ethoxy)silane

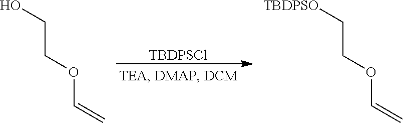

To a solution of 2-(vinyloxy)ethan-1-ol (20.0 g, 227.0 mmol) in DCM (200.0 mL) was added TEA (68.9 g, 681.00 mmol), DMAP (2.8 g, 22.70 mmol) and tert-butylchlorodiphenylsilane (74.9 g, 272.40 mmol) at room temperature under N₂. The resulting mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetate (10/1, v/v) to afford tert-butyldiphenyl(2-(vinyloxy)ethoxy)silane (40.0 g, 53%) as a yellow oil.

### Step 2: Synthesis of ethyl (trans)-2-(2-((tert-butyldiphenylsilyl)oxy)ethoxy)cyclopropane-1-carboxylate

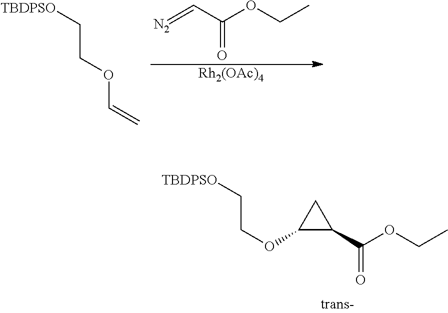

To a mixture of Rh₂(OAc)₄ (0.2 g, 0.46 mmol) in tert-butyldiphenyl(2-(vinyloxy)ethoxy)silane (5.0 g, 15.31 mmol) was added dropwise ethyl 2-diazoacetate (8.7 g, 76.57 mmol) at room temperature under N₂ in 10 h. The resulting mixture was stirred at room temperature for 16 h under N₂. After the reaction was completed, the resulting mixture was diluted with H₂O and extracted with CH₂Cl₂. The combined organic layer was washed with brine, dried over anhydrous sodium sulfate and filtered. The filtrate was concentrated under reduced pressure. The residue was purified by flash column chromatography with petroleum ether/ethyl acetated (10/1, v/v) to afford ethyl (trans)-2-(2-((tert-butyldiphenylsilyl)oxy)ethoxy)cyclopropane-1-carboxylate (2.3 g, 36%) as a colorless oil. LCMS (ESI, m/z): [M+H]⁺=413.2.

### Step 3: Synthesis of ethyl (trans)-2-(2-hydroxyethoxy)cyclopropane-1-carboxylate

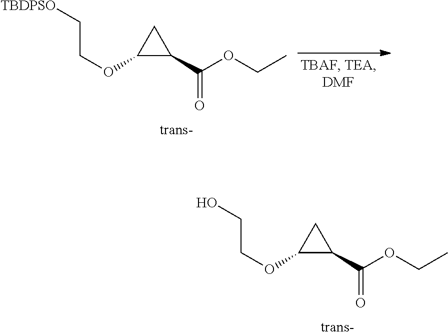

To a solution of ethyl (trans)-2-(2-((tert-butyldiphenylsilyl)oxy)ethoxy)cyclopropane-1-carboxylate (2.2 g, 5.33 mmol) in DMF (20.0 mL) was added TEA (1.6 g, 16.00 mmol) and TBAF (4.2 g, 16.00 mmol) at room temperature. The resulting mixture was stirred at 80° C. for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure to afford ethyl (trans)-2-(2-hydroxyethoxy)cyclopropane-1-carboxylate (1.3 g, crude) as a colorless oil. LCMS (ESI, m/z): [M+H]⁺=175.1.

### Step 4: Synthesis of ethyl (trans)-2-(2-oxoethoxy)cyclopropane-1-carboxylate

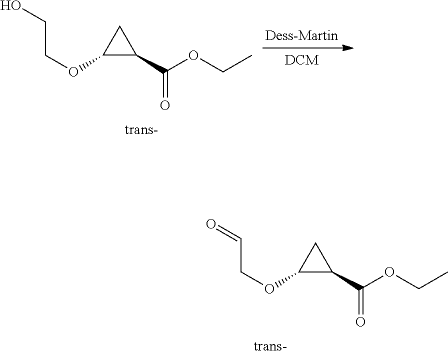

To a solution of ethyl (trans)-2-(2-hydroxyethoxy)cyclopropane-1-carboxylate (1.2 g, crude) in CH₂Cl₂ (30.0 mL) was added Dess-Martin (4.4 g, 10.33 mmol) at 0° C. The resulting mixture was stirred at room temperature for 1 h. After the reaction was completed, the reaction mixture was diluted with H₂O and extracted with CH₂Cl₂. The combined organic layer was washed with brine, dried over anhydrous sodium sulfate and filtered. The filtrate was concentrated under reduced pressure to afford ethyl (trans)-2-(2-oxoethoxy)cyclopropane-1-carboxylate (1.1 g, crude) as a yellow solid. LCMS (ESI, m/z): [M+H]⁺=173.1.

### Step 5: Synthesis of ethyl (trans)-2-(2-(dimethylamino)ethoxy)cyclopropane-1-carboxylate

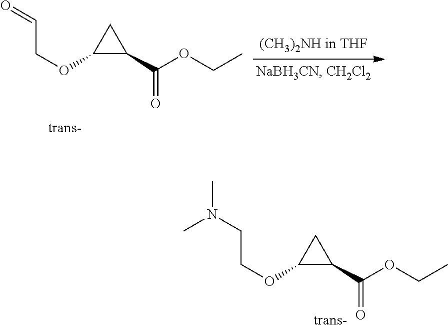

To a solution of ethyl (trans)-2-(2-oxoethoxy)cyclopropane-1-carboxylate (1.1 g, crude) in CH₂Cl₂ (20.0 mL) was added (CH₃)₂NH in THF (9.6 mL, 2 mol/L) and NaBH₃CN (1.2 g, 19.17 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with CH₂Cl₂/CH₃OH (10/1, v/v) to afford ethyl (trans)-2-(2-(dimethylamino)ethoxy)cyclopropane-1-carboxylate (300.0 mg, 23%) as a yellow oil. LCMS (ESI, m/z): [M+H]⁺=202.1.

### Step 6: Synthesis of (Trans)-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethoxy)cyclopropane-1-carboxamide

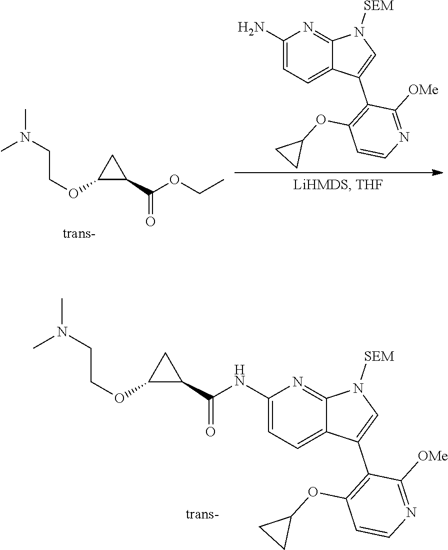 [SMILES: CCOC(=O)[C@@H]1C[C@H]1OCCN(C)C ; COc1nccc(OC2CC2)c1-c1cn(COCC[Si](C)(C)C)c2nc(N)ccc12 ; COc1nccc(OC2CC2)c1-c1cn(COCC[Si](C)(C)C)c2nc(NC(=O)[C@@H]3C[C@H]3OCCN(C)C)ccc12]

To a solution of ethyl (trans)-2-(2-(dimethylamino)ethoxy)cyclopropane-1-carboxylate (471.8 mg, 2.34 mmol) in THE (20.0 mL) was added 3-(4-cyclopropoxy-2-methoxypyridin-3-yl)-1-((2-(trimethylsilyl)ethoxy)methyl)-1H-pyrrolo[2,3-b]pyridin-6-amine (500.0 mg, 1.17 mmol) and LiHMDS (3.5 mL, 1 mol/L) at 0° C. under N₂. The resulting mixture was stirred at room temperature for 16 h. After the reaction was completed, the resulting mixture was quenched with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford (trans)-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethoxy)cyclopropane-1-carboxamide (180.0 mg, 26%) as a yellow solid. LCMS (ESI, m/z): [M+H]⁺=582.3.

### Step 7: Synthesis of Trans-N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide

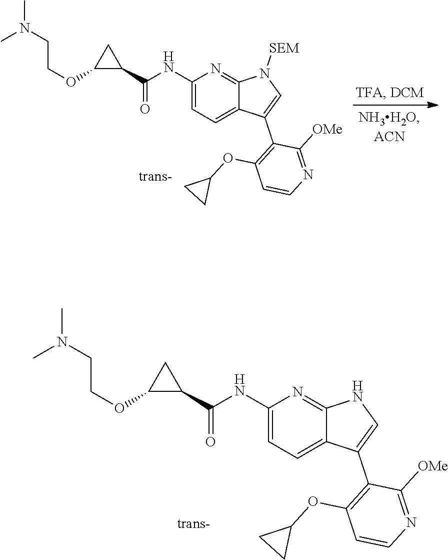 [SMILES: COc1nccc(OC2CC2)c1-c1cn(COCC[Si](C)(C)C)c2nc(NC(=O)[C@@H]3C[C@H]3OCCN(C)C)ccc12 ; COc1nccc(OC2CC2)c1-c1c[nH]c2nc(NC(=O)[C@@H]3C[C@H]3OCCN(C)C)ccc12]

To a solution of (trans)-N-(3-(4-cyclopropoxy-2-methoxypyridin-3-yl)-1-((2-(trimethylsilyl)ethoxy)methyl)-1H-pyrrolo[2,3-b]pyridin-6-yl)-2-(2-(dimethylamino)ethoxy)cyclopropane-1-carboxamide (500.0 mg, 0.86 mmol) in CH₂Cl₂ (5.0 mL) was added TFA (5.0 mL) at room temperature. The resulting mixture was stirred at room temperature for 3 h. The resulting mixture was concentrated under reduced pressure. To the residue in CH₃CN (5.0 mL) was added NH₃·H₂O (5.0 mL) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/CH₃OH (2/3, v/v) to afford trans-N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide (220.0 mg, 56%) as a white solid. LCMS (ESI, m/z): [M+H]⁺=452.2.

### Step 8: Synthesis of (1R,2R)—N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide and (1S,2S)—N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide (Compounds 251 and 252)

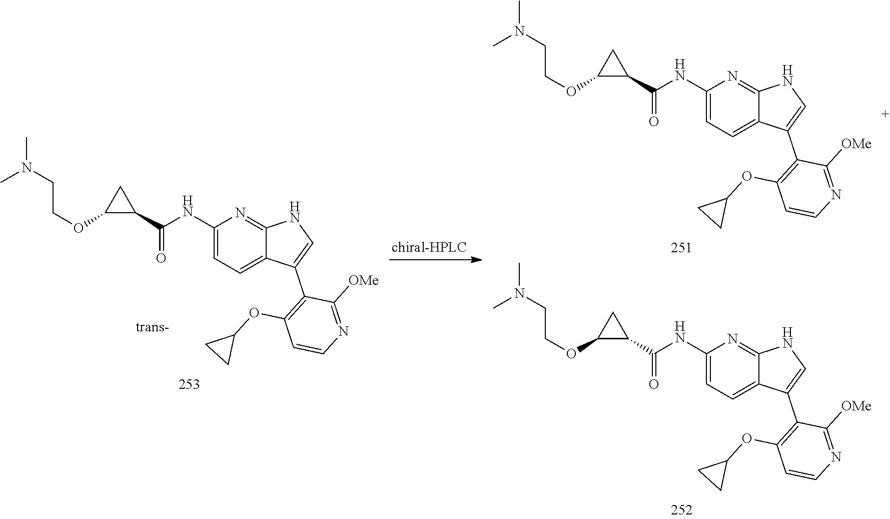 [SMILES: COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@H]3C[C@@H]3OCCN(C)C)ccc12]

The product of trans-N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide (220.0 mg, 0.49 mmol) was separated by Prep-Chiral-HPLC with the following conditions (Column: CHIRALPAK IG, 2×25 cm, 5 m; Mobile Phase A: Hex (0.5% 2M NH₃-MeOH)—HPLC, Mobile Phase B: MeOH: EtOH=1:1—HPLC; Flow rate: 20 mL/min; Gradient: 80% to 80% in 21 min; Wave Length: 220/254 nm; RT1 (min): 10.36; RT2 (min): 15.92) to afford N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide Enantiomer 1 (RT1: 10.36 min, 58.7 mg, 53%) as a white solid and N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide Enantiomer 2 (RT2: 15.92 min, 57.5 mg, 52%) as a white solid. The absolute stereochemistry of Enantiomers 1 and 2 was not assigned. The two enantiomeric structures that could be obtained from chiral separation of the enantiomeric mixture as described above are shown as Compounds 251 and 252 in Table 1.

N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide Enantiomer 1: RT1 (min): 10.36; LCMS (ESI, m/z): [M+H]⁺=452.3. ¹H NMR (400 MHz, DMSO-d₆): δ 11.53 (s, 1H), 10.61 (s, 1H), 8.08 (d, J=5.6 Hz, 1H), 7.82 (d, J=8.4 Hz, 1H), 7.56 (d, J=8.8 Hz, 1H), 7.35 (d, J=2.4 Hz, 1H), 7.14 (d, J=6.0 Hz, 1H), 3.95-3.91 (m, 1H), 3.81 (s, 3H), 3.64-3.52 (m, 3H), 2.61-2.53 (m, 1H), 2.22-2.18 (m, 7H), 1.16-1.13 (m, 2H), 0.82-0.78 (m, 2H), 0.68-0.59 (m, 2H).

N-(3-(4-cyclopropoxy-2-methoxypyridin-3-yl)-1H-pyrrolo[2,3-b]pyridin-6-yl)-2-(2-(dimethylamino)ethoxy)cyclopropane-1-carboxamide Enantiomer 2: RT2 (min): 15.92; LCMS (ESI, m/z): [M+H]⁺=452.3. ¹H NMR (400 MHz, DMSO-d₆): δ 11.53 (s, 1H), 10.61 (s, 1H), 8.08 (d, J=5.6 Hz, 1H), 7.82 (d, J=8.4 Hz, 1H), 7.56 (d, J=8.4 Hz, 1H), 7.35 (d, J=2.4 Hz, 1H), 7.14 (d, J=6.0 Hz, 1H), 3.95-3.91 (m, 1H), 3.81 (s, 3H), 3.63-3.52 (m, 3H), 2.49-2.47 (m, 2H), 2.21-2.15 (m, 7H), 1.16-1.13 (m, 2H), 0.82-0.73 (m, 2H), 0.68-0.59 (m, 2H).

### Example S221: Synthesis of (1R,2R)-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide and (1S,2S)-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide (Compound 254 and Compound 255)

### Step 1: Synthesis of ethyl (trans)-2-(2-(azetidin-1-yl)ethoxy)cyclopropane-1-carboxylate

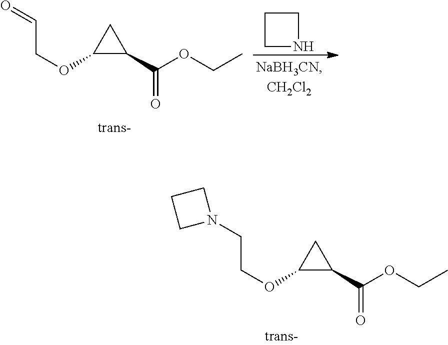

To a solution of ethyl (trans)-2-(2-oxoethoxy)cyclopropane-1-carboxylate (1.1 g, 6.39 mmol) in CH₂Cl₂ (20.0 mL) was added azetidine (1.1 g, 19.17 mmol) and NaBH₃CN (1.2 g, 19.17 mmol) at room temperature. The resulting mixture was stirred at room temperature for 2 h. After the reaction was completed, the resulting mixture was quenched with CH₃OH. The resulting mixture was concentrated under reduced pressure. The residue was purified by flash column chromatography with CH₂Cl₂/CH₃OH (10/1, v/v) to afford ethyl (trans)-2-(2-(azetidin-1-yl)ethoxy)cyclopropane-1-carboxylate (500.0 mg, 36%) as a yellow oil. LCMS (ESI, m/z): [M+H]⁺=214.1.

### Step 2: Synthesis of (Trans)-2-(2-(azetidin-1-yl)ethoxy)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide

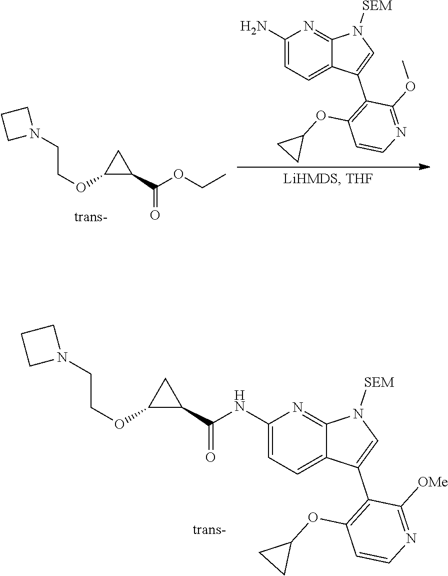 [SMILES: CCOC(=O)[C@@H]1C[C@H]1OCCN1CCC1 ; COc1nccc(OC2CC2)c1-c1cn(COCC[Si](C)(C)C)c2nc(N)ccc12 ; COc1nccc(OC2CC2)c1-c1cn(COCC[Si](C)(C)C)c2nc(NC(=O)[C@@H]3C[C@H]3OCCN3CCC3)ccc12]

To a solution of ethyl (trans)-2-(2-(azetidin-1-yl)ethoxy)cyclopropane-1-carboxylate (440.0 mg, 2.06 mmol) in THE (20.0 mL) was added 3-(4-cyclopropoxy-2-methoxypyridin-3-yl)-1-((2-(trimethylsilyl)ethoxy)methyl)-1H-pyrrolo[2,3-b]pyridin-6-amine (220.0 mg, 0.52 mmol) and LiHMDS (1.6 mL, 1 mol/L) at 0° C. under N₂. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by flash column chromatography with CH₂Cl₂/CH₃OH (10/1, v/v) to afford (trans)-2-(2-(azetidin-1-yl)ethoxy)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide (130.0 mg, 42%) as a yellow solid. LCMS (ESI, m/z): [M+H]⁺=594.3.

### Step 3: Synthesis of Trans-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide

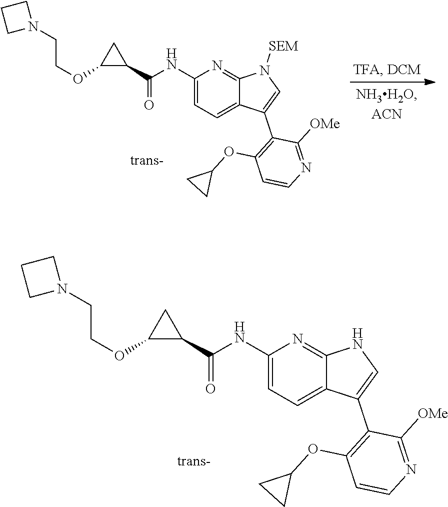 [SMILES: COc1nccc(OC2CC2)c1-c1cn(COCC[Si](C)(C)C)c2nc(NC(=O)[C@@H]3C[C@H]3OCCN3CCC3)ccc12 ; COc1nccc(OC2CC2)c1-c1c[nH]c2nc(NC(=O)[C@@H]3C[C@H]3OCCN3CCC3)ccc12]

To a solution of (trans)-2-(2-(azetidin-1-yl)ethoxy)-N-(3-(4-cyclopropoxy-2-methoxypyridin-3-yl)-1-((2-(trimethylsilyl)ethoxy)methyl)-1H-pyrrolo[2,3-b]pyridin-6-yl)cyclopropane-1-carboxamide (180.0 mg, 0.30 mmol) in CH₂Cl₂ (5.0 mL) was added TFA (5.0 mL) at room temperature. The resulting mixture was stirred at room temperature for 3 h. The resulting mixture was concentrated under reduced pressure. To the residue in CH₃CN (5.0 mL) was added NH₃·H₂O (5.0 mL) at room temperature. The resulting mixture was stirred at room temperature for 4 h. After the reaction was completed, the resulting mixture was diluted with H₂O and extracted with ethyl acetate. The combined organic layer was washed with brine, dried over anhydrous sodium sulfate and filtered. The filtrate was concentrated under reduced pressure. The residue was purified by reverse phase flash column chromatography with H₂O/CH₃CN (3/2, v/v) to afford trans-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide (49.0 mg, 34%) as a white solid. LCMS (ESI, m/z): [M+H]⁺=464.2.

### Step 4: Synthesis of (1R,2R)-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide and (1S,2S)-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide (Compound 254 and Compound 255)

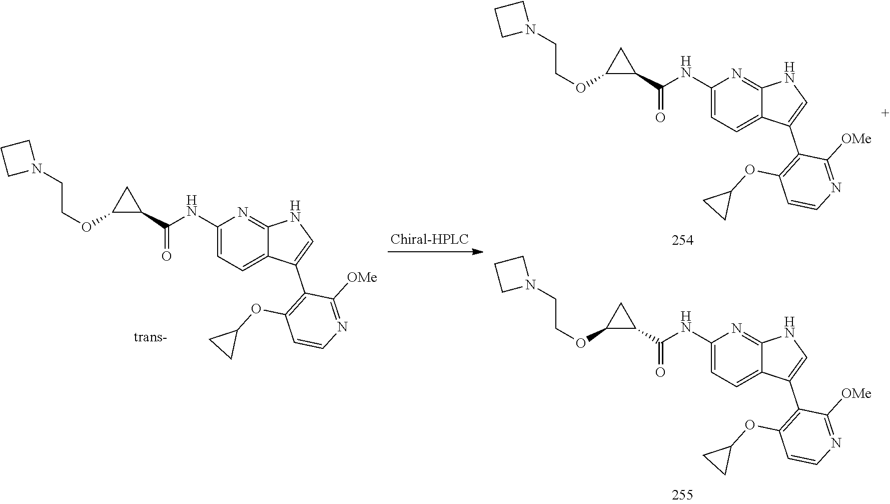 [SMILES: COc1nccc(OC2CC2)c1-c1c[nH]c2nc(NC(=O)[C@@H]3C[C@H]3OCCN3CCC3)ccc12 ; COc1nccc(OC2CC2)c1-c1c[nH]c2nc(NC(=O)[C@@H]3C[C@H]3OCCN3CCC3)ccc12 ; COc1nccc(OC2CC2)c1-c1c[nH]c2nc(NC(=O)[C@H]3C[C@@H]3OCCN3CCC3)ccc12]

The product of trans-2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide (48.0 mg, 0.10 mmol) was separated by Prep-Chiral-HPLC with the following conditions (Column: CHIRALPAK IG, 2×25 cm, 5 m; Mobile Phase A: Hex (0.5% 2M NH₃-MeOH)—HPLC, Mobile Phase B: EtOH: DCM=1: 1—HPLC; Flow rate: 20 mL/min; Gradient: 45% B to 45% B in 24.5 min; Wave Length: 220/254 nm; RT1 (min): 9.43; RT2 (min): 17.99) to afford 2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide Enantiomer 1 (12.8 mg, 53%) as a white solid and 2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide Enantiomer 2 (11.6 mg, 48%) as a white solid. The absolute stereochemistry of Enantiomers 1 and 2 was not assigned. The two enantiomeric structures that could be obtained from chiral separation of the enantiomeric mixture as described above are shown as Compounds 254 and 255 in Table 1.

2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide Enantiomer 1: RT1 (min): 9.43; LCMS (ESI, m/z): [M+H]⁺=464.2. ¹H NMR (400 MHz, DMSO-d₆): δ 11.54 (s, 1H), 10.61 (s, 1H), 8.08 (d, J=6.0 Hz, 1H), 7.82 (d, J=8.8 Hz, 1H), 7.56 (d, J=8.4 Hz, 1H), 7.35 (d, J=2.4 Hz, 1H), 7.14 (d, J=5.6 Hz, 1H), 3.94-3.92 (m, 1H), 3.82 (s, 3H), 3.58-3.45 (m, 4H), 3.19-3.13 (m, 4H), 2.24-2.18 (m, 1H), 1.99-1.92 (m, 2H), 1.14-1.11 (m, 2H), 0.81-0.77 (m, 2H), 0.67-0.63 (m, 2H).

2-(2-(azetidin-1-yl)ethoxy)-N-(3-(4-cyclopropoxy-2-methoxypyridin-3-yl)-1H-pyrrolo[2,3-b]pyridin-6-yl)cyclopropane-1-carboxamide Enantiomer 2: RT2 (min): 17.99; LCMS (ESI, m/z): [M+H]⁺=464.3. ¹H NMR (400 MHz, DMSO-d₆): δ 11.54 (s, 1H), 10.61 (s, 1H), 8.08 (d, J=5.6 Hz, 1H), 7.82 (d, J=8.8 Hz, 1H), 7.56 (d, J=8.4 Hz, 1H), 7.35 (d, J=2.4 Hz, 1H), 7.14 (d, J=6.0 Hz, 1H), 3.94-3.92 (m, 1H), 3.82 (s, 3H), 3.52-3.44 (m, 3H), 3.18-3.11 (m, 4H), 2.24-2.18 (m, 1H), 1.98-1.91 (m, 2H), 1.14-1.11 (m, 2H), 0.81-0.77 (m, 2H), 0.74-0.64 (m, 2H).

### Biological Examples

### K562 and HL60 Cell Proliferation Assay

K562 and HL60 cells, cultured in Iscove's Modified Dulbecco's Media (IMDM) supplemented with 10% FBS, were harvested at 50-80% confluency and plated at 2,000 cells per well (K562) or 1,500 cells per well (HL60) in 384-well tissue culture plates. A subset of wells contained media only (low control, LC). Compounds were serially diluted in DMSO. 40 nL of compound or DMSO only (high control, HC) were added to each well using an Echo 550 liquid handler (Labcyte). The plates were placed in a 37° C. incubator with 5% CO₂ for 72 hours. Cell viability was measured using a CellTiter-Glo luminescent cell viability assay (Promega), which allows for relative quantification of metabolically active cells by luminescence-based measurements of intracellular ATP concentrations. Briefly, plates were removed from the incubator and equilibrated for 15 minutes at room temperature prior to the addition of 40 μL of CellTiter-Glo reagent. Plates were then incubated for 30 minutes at room temperature. Luminescence was measured using an EnSpire plate reader (Perkin Elmer). As noted above, luminescence values from wells with DMSO only or media without cells were used as high and low controls (HC and LC), respectively. Normalized percent viability was calculated as follows: percent viability=100×(Lumₛₐₘₚₗₑ−Lum_{LC})/(Lum_{HC}−Lum_{LC}). IC₅₀ values were calculated using the XLFit software and are shown in Table 2.

TABLE 2

Cell Line (IC₅₀, nM)

| Cmpd No. | K562 | HL60 |
|---|---|---|
| 1 | 101 | >10.0E+03 |
| 2 | 176 | >10.0E+03 |
| 3 | 80.3 | >10.0E+03 |
| 4 | 223 | >10.0E+03 |
| 5 | 713 | >10.0E+03 |
| 6 | 452 | >10.0E+03 |
| 13 | 202 | 4450 |
| 14 | 416 | >10.0E+03 |
| 15 | 181 | 1710 |
| 17 | 229 | >10.0E+03 |
| 19 | 147 | 287 |
| 21 | 286 | 1570 |
| 22 | 421 | >10.0E+03 |
| 26 | 220 | >10.0E+03 |
| 27 | 156 | >10.0E+03 |
| 28 | 62.1 | 385 |
| 29 | 454 | 470 |
| 37 | 231 | >10.0E+03 |
| 41 | 654 | >10.0E+03 |
| 42 | 254 | >10.0E+03 |
| 43 | 779 | >10.0E+03 |
| 44 | 151 | >10.0E+03 |
| 45 | 52.7 | >10.0E+03 |
| 47 | 292 | >10.0E+03 |
| 48 | 777 | 9210 |
| 49 | 295 | >10.0E+03 |
| 50 | 120 | >10.0E+03 |
| 51 | 668 | >10.0E+03 |
| 52 | 829 | >10.0E+03 |
| 53 | 267 | 3110 |
| 54 | 88.4 | >10.0E+03 |
| 56 | 183 | 120 |
| 58 | 132 | >10.0E+03 |
| 60 | 5170 | >10.0E+03 |
| 61 | 36.9 | >10.0E+03 |
| 62 | 137 | >10.0E+03 |
| 63 | 255 | >10.0E+03 |
| 64 | 610 | >10.0E+03 |
| 66 | 603 | >10.0E+03 |
| 67 | 1140 | >10.0E+03 |
| 68 | 607 | 3690 |
| 69 | 162 | >10.0E+03 |
| 70 | 312 | >10.0E+03 |
| 71 | 292 | 334 |
| 72 | 1020 | >10.0E+03 |
| 74 | 351 | >10.0E+03 |
| 75 | 54.6 | >10.0E+03 |
| 76 | 35.8 | >10.0E+03 |
| 77 | 727 | >10.0E+03 |
| 78 | 94.8 | >10.0E+03 |
| 79 | 719 | >10.0E+03 |
| 81 | 96.2 | >10.0E+03 |
| 82 | 437 | >10.0E+03 |
| 83 | 48.8 | >10.0E+03 |
| 84 | 150 | >10.0E+03 |
| 85 | 46 | 7250 |
| 86 | 30.5 | >10.0E+03 |
| 87 | 94.1 | >10.0E+03 |
| 88 | 84.1 | >10.0E+03 |
| 90 | 1100 | >10.0E+03 |
| 91 | 1080 | >10.0E+03 |
| 92 | 248 | >10.0E+03 |
| 94 | 55.6 | 7580 |
| 95 | 221 | >10.0E+03 |
| 96 | 862 | >10.0E+03 |
| 98 | 106 | >10.0E+03 |
| 99 | 129 | >10.0E+03 |
| 101 | 94.2 | >10.0E+03 |
| 102 | 48 | >10.0E+03 |
| 103 | 751 | >10.0E+03 |
| 105 | 489 | >10.0E+03 |
| 106 | 275 | >10.0E+03 |
| 108 | 419 | >10.0E+03 |
| 109 | 278 | >10.0E+03 |
| 110 | 382 | >10.0E+03 |
| 111 | 321 | >10.0E+03 |
| 112 | 1860 | >10.0E+03 |
| 113 | 174 | >10.0E+03 |
| 115 | 382 | >10.0E+03 |
| 116 | 142 | >10.0E+03 |
| 117 | 67.1 | >10.0E+03 |
| 118 | 24.4 | >10.0E+03 |
| 119 | 32.2 | >10.0E+03 |
| 121 | 365 | >10.0E+03 |
| 124 | 359 | >10.0E+03 |
| 125 | 104 | >10.0E+03 |
| 126 | 2010 | >10.0E+03 |
| 127 | 100 | >10.0E+03 |
| 130 | 169 | >10.0E+03 |
| 131 | 83.3 | >10.0E+03 |
| 132 | 234 | >10.0E+03 |
| 133 | 9.36 | 4040 |
| 135 | 165 | >10.0E+03 |
| 136 | 333 | >10.0E+03 |
| 137 | 733 | >10.0E+03 |
| 138 | 527 | >10.0E+03 |
| 139 | 1380 | >10.0E+03 |
| 142 | 34.5 | >10.0E+03 |
| 144 | 32 | >10.0E+03 |
| 145 | 120 | >10.0E+03 |
| 146 | 9.9 | >10.0E+03 |
| 147 | 16.1 | >10.0E+03 |
| Example S150, Enantiomer 1 | 40.5 | — |
| 153 | 8.67 | >10.0E+03 |
| Example S205, Enantiomer 1 | 21.5 | — |
| Example S205, Enantiomer 2 | 55.3 | — |
| Example S220, Enantiomer 1 | 24.9 | >10.0E+03 |
| Example S220, Enantiomer 2 | 57.3 | >10.0E+03 |
| 253 | 30.8 | >10.0E+03 |
| Example S221, Enantiomer 1 | 11.1 | 6590 |
| Example S221, Enantiomer 2 | 39.7 | 8660 |

### Luminescence-Based ABL Kinase Assay (300 μM ATP)

Kinase activity of ABL1 was measured using the ADP-Glo system (Promega), which measures formation of ADP using a luminescence-based method. Compounds were serially diluted in DMSO. 20 nL of compound or DMSO only (high control, HC) were added to a 384-well plate (OptiPlate-384, PerkinElmer) using an Echo550 liquid handler (Labcyte). 15 μL of kinase solution (10 mM MgCl₂, 0.01% Brij-35, 2 mM DTT, 0.05% BSA, 1 mM EGTA, 50 mM HEPES pH 7.5, and 3.325 nM ABL1 [Carna Biosciences]) were added to each well of the 384-well plate containing the compounds. No enzyme control wells were included (low control, LC). The plate was incubated at room temperature for 30 minutes. 5 μL of a second solution containing 10 mM MgCl₂, 0.01% Brij-35, 2 mM DTT, 0.05% BSA, 1 mM EGTA, 50 mM HEPES pH 7.5, 6 μM Peptide 2 (Perkin Elmer, Cat No 760346), and 1.2 mM ATP were added to each well to start the kinase reaction. The plate was incubated for 90 minutes at room temperature. 20 μL of ADP-Glo reagent (Promega) were then added to each well and the plate was incubated for 40 minutes at room temperature. 40 μL of kinase detection reagent (Promega) was added to each well and the plate was incubated for an additional 45 minutes at room temperature. During this step, ADP was converted to ATP, a substrate for luciferase, to produce luminescence signal. Luminescence was measured on an Envision plate reader (Perkin Elmer). Luminescence signal positively correlates with kinase activity. The percent kinase activity was calculated as follows: percent kinase activity=100×(Lumₛₐₘₚₗₑ−Lum_{LC})/(Lum_{HC}−Lum_{LC}). As noted above, DMSO only and no enzyme wells were used as high and low controls, respectively. IC50 values were calculated using the XLFit software.

IC₅₀ data obtained using the screening procedures described above for certain compounds disclosed herein are listed in Table 3.

TABLE 3

Kinase, IC₅₀ (nM)

| Cmpd No. | ABL1 | ABL1 (T315I) |
|---|---|---|
| 1 | 37.9 | 11.3 |
| 2 | 22.2 | 9.96 |
| 3 | 8.17 | 0.896 |
| 4 | 113 | 32.3 |
| 5 | 119 | 32.9 |
| 6 | 123 | 65.6 |
| 13 | 96.6 | 9.74 |
| 14 | 91.2 | 52.8 |
| 15 | 12.1 | 2.78 |
| 16 | >10.0E+03 | >10.0E+03 |
| 17 | 55.1 | 28.6 |
| 18 | 33 | 14.5 |
| 19 | 11.2 | 4.57 |
| 20 | 110 | 27.7 |
| 21 | 94.8 | 8.74 |
| 22 | 78.9 | 7.17 |
| 24 | 149 | 33 |
| 25 | 776 | 74.3 |
| 26 | 62.9 | 68.4 |
| 27 | 29 | 20.2 |
| 28 | 7.58 | 2.84 |
| 29 | 41.8 | 10.5 |
| 30 | 148 | 90.2 |
| 31 | 169 | 359 |
| 32 | 135 | 183 |
| 33 | 319 | 603 |
| 34 | 159 | 731 |
| 35 | 599 | 774 |
| 36 | 202 | 690 |
| 37 | 46.1 | 103 |
| 38 | 689 | 332 |
| 39 | 195 | 245 |
| 40 | 121 | 170 |
| 41 | 106 | 26.8 |
| 42 | 45.4 | 12.7 |
| 43 | 91.1 | 15.6 |
| 44 | 23.1 | 39.8 |
| 45 | 4.64 | 4.62 |
| 46 | 86.4 | 20.6 |
| 47 | 7.47 | 0.705 |
| 48 | 94.9 | 244 |
| 49 | 4.33 | 3.04 |
| 50 | 3.9 | 2.51 |
| 51 | 15.4 | 4.41 |
| 52 | 8.95 | 13.7 |
| 53 | 6.92 | 5.63 |
| 54 | 14.3 | 16.3 |
| 55 | 114 | 1030 |
| 56 | 30.3 | 53.5 |
| 57 | 1090 | 3870 |
| 58 | 2.92 | 3.4 |
| 59 | 8.62 | 11 |
| 60 | 218 | 417 |
| 61 | 3.28 | 5.08 |
| 62 | 20.8 | 43 |
| 63 | 40.7 | 64.5 |
| 64 | 56.9 | 140 |
| 65 | 158 | 8700 |
| 66 | 91.5 | 38.7 |
| 67 | 157 | 37.7 |
| 68 | 78.2 | 19.2 |
| 69 | 27.6 | 27.4 |
| 70 | 60.3 | 140 |
| 71 | 48 | 55.2 |
| 72 | 70.2 | 162 |
| 73 | 2460 | >10.0E+03 |
| 74 | 55.6 | 63 |
| 75 | 15.1 | 17.6 |
| 76 | 5.14 | 7.38 |
| 77 | 106 | 137 |
| 78 | 17.8 | 139 |
| 79 | 143 | 108 |
| 80 | 1050 | 659 |
| 81 | 12.2 | 12.9 |
| 82 | 13.5 | 20.5 |
| 83 | 1.52 | 1.74 |
| 84 | 37 | 14.9 |
| 85 | 5.34 | 27.8 |
| 86 | 5.97 | 5.48 |
| 87 | 9.54 | 6.59 |
| 88 | 18.6 | 30.9 |
| 89 | 337 | 219 |
| 90 | 21.7 | 15 |
| 91 | 166 | 24.6 |
| 92 | 39.4 | 27.3 |
| 93 | 5.3 | 2.94 |
| 94 | 10.8 | 6.36 |
| 95 | 6.32 | 2.14 |
| 96 | 8.87 | 8.93 |
| 97 | 199 | 61.6 |
| 98 | 16.4 | 12.9 |
| 99 | 14.9 | 25.7 |
| 100 | 98.2 | 105 |
| 101 | 16.4 | 15.3 |
| 102 | 12.5 | 17.8 |
| 103 | 99.2 | 32.5 |
| 104 | 102 | 29.1 |
| 105 | 109 | 19.9 |
| 106 | 78.1 | 26.1 |
| 107 | 151 | 19.5 |
| 108 | 78.6 | 17.8 |
| 109 | 39 | 7.08 |
| 110 | 74.8 | 18.2 |
| 111 | 14.3 | 151 |
| 112 | 271 | 157 |
| 113 | 20.9 | 15.9 |
| 114 | 51.4 | 150 |
| 115 | 124 | 151 |
| 116 | 49.4 | 59.3 |
| 117 | 11.3 | 9.08 |
| 118 | 3.73 | 1.48 |
| 119 | 4.67 | 2.57 |
| 120 | 65.5 | 60.8 |
| 121 | 31.8 | 5.98 |
| 122 | 73.4 | 19.9 |
| 123 | 56.5 | 17.6 |
| 124 | 317 | 30.6 |
| 125 | 22.2 | 39.5 |
| 126 | 2580 | 3860 |
| 127 | 22.1 | 0.745 |
| 128 | 24.9 | 97.1 |
| 129 | 32.9 | 35.8 |
| 130 | 67 | 33.7 |
| 131 | 42.7 | 28.1 |
| 132 | 143 | 97.5 |
| 133 | 3.57 | 6.1 |
| 134 | 64.8 | 227 |
| 135 | 24.9 | 15.8 |
| 136 | 27.4 | 54.5 |
| 137 | 36 | 40.6 |
| 138 | 51.2 | 140 |
| 139 | 18.6 | 16.8 |
| 140 | 30.6 | 82.5 |
| 141 | 45.5 | 31.2 |
| 142 | 14.6 | 14.3 |
| 145 | 51.6 | 44.3 |
| 146 | 3.74 | 14 |
| 147 | 5.54 | 1.71 |
| Example S148, Enantiomer 1 | 408 | 1810 |
| Example S150, Enantiomer 1 | 5.42 | 1.73 |
| Example S150, Enantiomer 2 | 30 | 27.2 |
| 153 | 4.65 | 1.37 |
| Example S205, Enantiomer 1 | 10.6 | 15 |
| Example S205, Enantiomer 2 | 15 | 20.9 |
| 250 | 411 | 408 |
| 253 | 6.71 | 8.81 |

pCRKL ELISA assay:

K562 or Ba/F3 ABL T315I cells (2.0*10⁵ cells/100 μl/well) were seeded in 96 well (Corning, cat #3799). Compounds were dissolved in DMSO, serially diluted in DMSO and then were added, mixed, and incubated for 90 minutes at 37° C., 5% CO₂. Following the 90-minute incubation, plates were centrifuged for 5 min at 3000 RPM and supernatant was removed from each well. Cells were washed 3 times with 150 μl PBS prior to addition of 100 μl cell RIPA lysis buffer (Boston BioProducts, cat #BP-115D) supplied with 1×complete ULTRA cocktail inhibitor (Roche, 05892791001) and 1×PhosSTOP Phosphatase Inhibitor Cocktail Tablets (Roche, 04906837001). Cells were incubated with lysis buffer for 1 hour at 4° C. prior to storage at −80 C.

A capture antibody able to detect phosphorylated and non-phosphorylated CRKL (R&D Systems, cat #AF5127) was added to Meso Scale Discovery (MSD) standard bind plates (MSD, cat #L15XA-3) at 5 ug/mL and incubated at 4° C. overnight. The next day, plates were washed with PBS+0.05% Tween20 (PBST) and 150 μl of 5% BSA blocking solution was added for 1 hour at room temperature with shaking. Plates were washed with PBST. Lysates were thawed and 30 μl of lysate was added to the MSD plates and incubated for 2 hours at room temperature with shaking. MSD plates were washed with PBST and 30 μl of a detection antibody that binds phosphorylated pCRKL (R&D Systems, cat #MAB6910) was added at 1 μg/mL to each well. Plates were incubated for 1 hour at room temperature with shaking. Plates were washed with PBST prior to addition of 30 μl of a sulfo tagged goat anti mouse detection antibody (MSD cat #R32AC-1). Plates were incubated for 1 hour at room temperature with shaking. Plates were washed with PBST prior to addition of 150 ul of 1×MSD read buffer T (MSD, cat 4R92TC-2). Electrochemiluminescence (ECLU) was read on an MSD plate reader (Meso Scale Discovery). The remaining activity by calculated as follows: 00 Relative activity=100×(ELCUₛₐₘₚₗₑ−ECLU_{LC})/(ECLU_{HC}−ECLU_{LC}). The low and high controls (LC/HC) are generated from lysate from wells without cells or with cells treated with 0.1% DMSO, respectively. IC50 values were calculated using XLFit software using a nonlinear regression model with a sigmoidal dose response and are shown in Table 4 and Table 5 below.

TABLE 4

| | PCRKL ELISA, Cell |
|---|---|
| | Line IC₅₀ (nM) |
| Cmpd No. | K562 |
| 3 | 174 |
| 15 | 468 |
| 26 | 680 |
| 27 | 164 |
| 42 | 88.3 |
| 44 | 275 |
| 47 | 685 |
| 49 | 585 |
| 50 | 151 |
| 51 | 1350 |
| 52 | 3120 |
| 53 | 584 |
| 54 | 95.8 |
| 56 | 745 |
| 58 | 281 |
| 61 | 88.4 |
| 62 | 171 |
| 63 | 328 |
| 66 | 702 |
| 74 | 572 |
| 75 | 86.3 |
| 76 | 65.4 |
| 77 | 1120 |
| 82 | 1050 |
| 95 | 656 |
| 98 | 174 |
| 131 | 272 |
| 133 | 278 |
| 135 | 1210 |
| 145 | 399 |
| 146 | 32 |
| 147 | 35.7 |
| Example S150, Enantiomer 1 | 453 |

TABLE 5

| | pCRKL ELISA, Cell |
|---|---|
| | Line IC₅₀ (nM) |
| Compound No. | Ba/F3 ABL1 (T315I) |
| 27 | 183 |
| 54 | 198 |
| 75 | 162 |
| 76 | 50.1 |
| 86 | 84.2 |
| 95 | 429 |
| 98 | 241 |
| 116 | 1060 |
| 117 | 124 |
| 146 | 112 |
| 147 | 35.7 |
| Example S150, Enantiomer 1 | 1460 |

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A compound of formula (I) 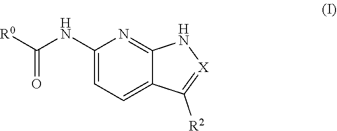
or a pharmaceutically acceptable salt, solvate, hydrate, or co-crystal thereof, or a mixture of any of the foregoing, wherein:
X is N or CR⁸;
R⁰ is a group 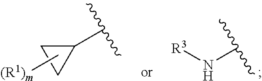
m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, —O—C₁-C₃ alkylene-NR⁴R⁵, —O—C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(4- to 8-membered heterocycloalkylene)-(C₀-C₂ alkylene)-NR⁴R⁵, C₁-C₂ alkylene-(4- to 8-membered heterocycloalkylene)-(C₀-C₂ alkylene)-NR^{4′}R⁵, C₁-C₂ alkylene-(C₃-C₇ heterocycloalkylene)-(C₀-C₂ alkylene)-NR⁴R⁵ or C₁-C₂ alkylene-(C₃-C₇ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl:
or
two R¹ are taken together with the carbon atom or carbon atoms to which they are attached to form a 3- to 7-membered heterocyclic ring, wherein the heterocyclic ring contains nitrogen atom and wherein the nitrogen atom is optionally substituted with C₁-C₃ alkyl;
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′} R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(4- to 8-membered heterocycloalkyl), C₁-C₃ alkylene-(C₃-C₇ heterocycloalkyl), C₁-C₃ alkylene-(4- to 8-membered heterocycloalkylene)-(C₀-C₂ alkylene)-NR⁴R⁵, C₁-C₃ alkylene-(4- to 8-membered heterocycloalkylene)-(C₀-C₂ alkylene)-NR^{4′}R^{5′}, (4- to 8-membered heterocycloalkylene)-(C₀-C₂ alkylene)-NR⁴R⁵, C₁-C₃ alkylene-(C₃-C₇ heterocycloalkylene)-(C₀-C₂ alkylene)-NR⁴R⁵, C₁-C₃ alkylene-(C₃-C₇ heterocycloalkylene)-(C₀-C₂ alkylene)-NR^{4′}R^{5′}, or (C₃-C₇ heterocycloalkylene)-(C₀-C₂ alkylene)-NR⁴R⁵, wherein the alkyl, alkylene, cycloalkyl, cycloalkylene, heterocycloalkyl, and heterocycloalkylene moieties in R³ are optionally substituted with 1-3 fluorine atoms, 1-3 CN groups and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, 4- to 8-membered heterocycloalkyl, or C₃-C₇ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl, wherein said C₁-C₃ alkyl is optionally substituted with 1-6 deuterium atoms;
each pair of R^{4′} and R^{5′} taken together with the nitrogen atom to which they are attached independently form a 3-to-7-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O), C₁-C₃ alkyl, or S(O)ₙC₃-C₆ cycloalkyl,
wherein n is an integer from 0 to 2;
each R⁷ is independently —H, C₁-C₃ alkyl, C₁-C₃ haloalkyl, C₃-C₆ cycloalkyl, or C₁-C₃-alkylene-C₃-C₆ cycloalkyl, wherein said C₁-C₃ alkyl is optionally substituted with 1-6 deuterium atoms; and
R⁸ is —H, —F, or C₁-C₃ alkyl.

2. A compound of formula (I) 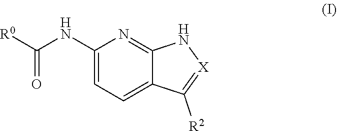
or a pharmaceutically acceptable salt, solvate, hydrate, or co-crystal thereof, or a mixture of any of the foregoing, wherein:
X is N or CR⁸;
R⁰ is a group 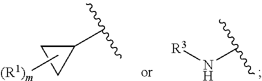
m is an integer from 0 to 3;
each R¹ is independently -D, —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R⁵, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
R² is C₆-C₁₄ aryl or 5-to-10-membered heteroaryl, wherein the C₆-C₁₄ aryl and 5-to-10-membered heteroaryl are optionally substituted with 1-5 R⁶ groups;
R³ is —H, C₁-C₆ alkyl, C₃-C₆ cycloalkyl, C₁-C₆ alkylene-NR⁴R⁵, C₁-C₆ alkylene-NR^{4′}R^{5′}, C₁-C₆alkylene-OH, C₁-C₃ alkylene-CN, C₁-C₃ alkylene-(C₃-C₆ cycloalkyl), C₁-C₃ alkylene-(C₃-C₆ cycloalkylene)-(C₀-C₂ alkylene)-NR⁴R⁵, C₁-C₃ alkylene-(C₃-C₆ cycloalkylene)-(C₀-C₂ alkylene)-NR^{4′} R^{5′}, C₁-C₃ alkylene-O—(C₁-C₂ alkylene)-NR⁴R⁵, C₁-C₃ alkylene-O—(C₁-C₃ alkylene)-NR^{4′}R^{5′}, C₁-C₃ alkylene-(C₃-C₆ cycloalkylene)-(C₀-C₂ alkylene)-OH, C₁-C₃ alkylene-(C₄-C₆ heterocycloalkyl), C₁-C₃ alkylene-(C₄-C₆ heterocycloalkylene)-(C₀-C₂ alkylene)-NR⁴R⁵, or C₁-C₃ alkylene-(C₄-C₆ heterocycloalkylene)-(C₀-C₂ alkylene)-NR^{4′}R^{5′}, wherein the alkyl, alkylene, cycloalkyl, cycloalkylene, heterocycloalkyl, and heterocycloalkylene moieties in R³ are optionally substituted with 1-3 fluorine atoms and/or 1-6 deuterium atoms and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, C₂-C₃ heteroalkyl, or C₄-C₆ heterocycloalkyl;
each R⁴ is independently —H, C₁-C₆ alkyl, C₃-C₆ cycloalkyl, C₁-C₆ haloalkyl, C₁-C₆ alkylene-CN, or C₁-C₆ heteroalkyl;
each R⁵ is independently —H, C₁-C₆ alkyl, C₃-C₆ cycloalkyl, C₁-C₆ haloalkyl, C₁-C₆ alkylene-CN, or C₁-C₆ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N, O, and S, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, —NR^{4′}R^{5′}, C₁-C₃ alkyl, C₁-C₃ haloalkyl, C₃-C₆ cycloalkyl, —CN, S(O), C₁-C₃ alkyl, or S(O)ₙC₃-C₆ cycloalkyl,
wherein n is an integer from 0 to 2;
each R⁷ is independently —H, C₁-C₃ alkyl, C₁-C₃ haloalkyl, or C₃-C₆ cycloalkyl, wherein said C₁-C₃ alkyl is optionally substituted with 1-6 deuterium atoms; and
R⁸ is —H, —F, or C₁-C₃ alkyl.

3. The compound of claim 1, or a pharmaceutically acceptable salt, solvate, hydrate, or co-crystal thereof, or a mixture of any of the foregoing, wherein the compound of formula (I) is a compound of formula (I-A)

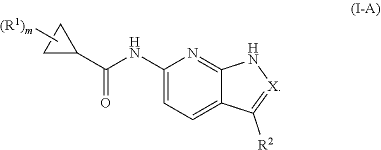

4. The compound of claim 1, or a pharmaceutically acceptable salt, solvate, hydrate, or co-crystal thereof, or a mixture of any of the foregoing, wherein the compound of formula (I) is a compound of formula (I-A-i) or formula (I-A-ii) 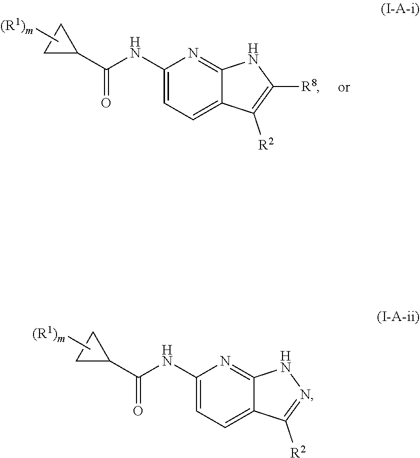
wherein:
m is an integer from 0 to 2;
each R¹ is independently —F, C₁-C₃ alkyl, C₁-C₃ alkylene-NR⁴R⁵, C₁-C₃ alkylene-NR^{4′}R^{5′}, C₁-C₃ alkylene-OH, C₀-C₃ alkylene-CN, C₁-C₂ alkylene-(C₃-C₆ cycloalkylene)-(C₀-C₂ alkylene)-NR⁴R⁵, C₁-C₂ alkylene-(C₃-C₆ cycloalkylene)-(C₀-C₂ alkylene)-NR^{4′}R^{5′}, C₁-C₂ alkylene-(C₃-C₆ cycloalkylene)-(C₀-C₂ alkylene)-OH, C₁-C₂ alkylene-(C₄-C₆ heterocycloalkylene)-(C₀-C₂ alkylene)-NR⁴R⁵ or C₁-C₂ alkylene-(C₄-C₆ heterocycloalkylene)-(C₀-C₂ alkylene)-NR^{4′}R^{5′}, wherein the alkyl, alkylene, cycloalkylene, and heterocycloalkylene moieties in R¹ are optionally substituted with 1-3 fluorine atoms and/or 1-6 deuterium atoms, and wherein each heterocyclic nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
R² is C₆-C₁₄ aryl or 5-to-10-membered heteroaryl, wherein said 5-to-10-membered heteroaryl is selected from the group consisting of 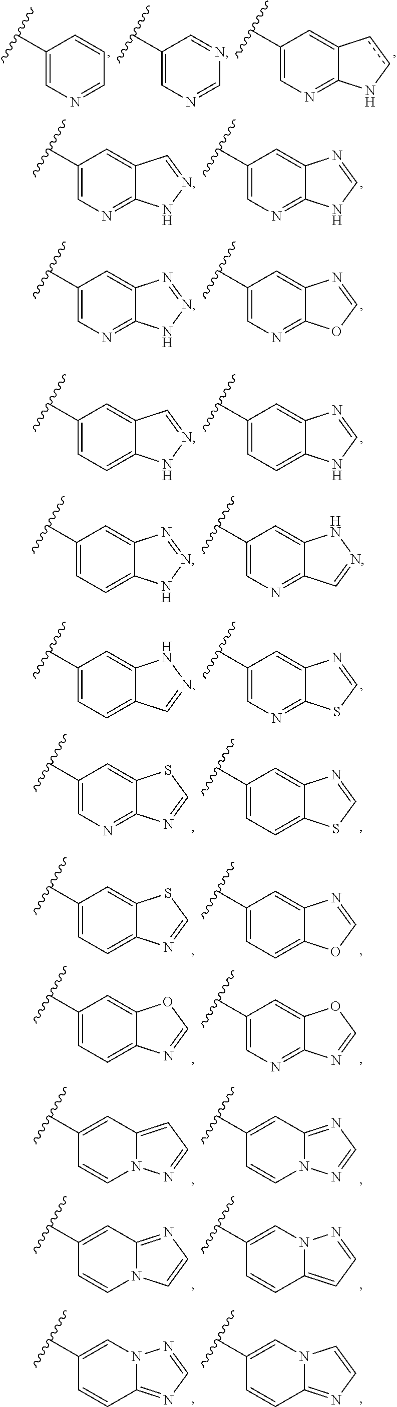 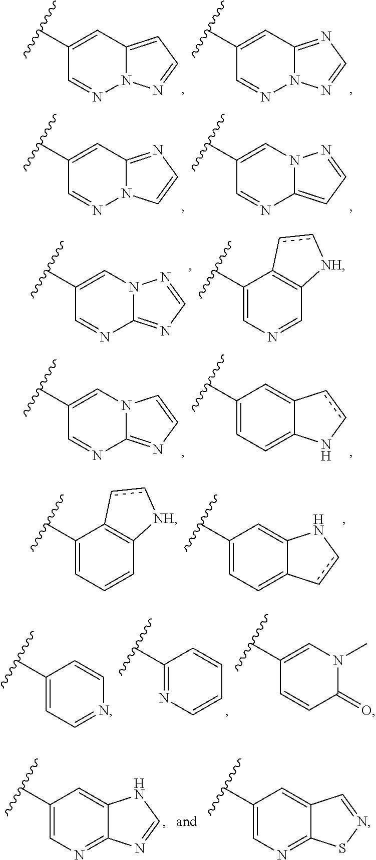
wherein [symbol] indicates a single or double bond, and wherein the C₆-C₁₄ aryl and 5-to-10-membered heteroaryl are optionally substituted with 1-5 R⁶ groups;
each R⁴ is independently —H, C₁-C₃ alkyl, C₃-C₆ cycloalkyl, C₂-C₃ haloalkyl, C₂-C₃ alkylene-CN, or C₂-C₃ heteroalkyl;
each R⁵ is independently —H, C₁-C₃ alkyl, C₃-C₆ cycloalkyl, C₂-C₃ haloalkyl, C₂-C₃ alkylene-CN, or C₂-C₃ heteroalkyl;
each pair of R^{4′} and R^{5′} taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl, C₃-C₆ cycloalkyl, C₂-C₃ haloalkyl, C₂-C₃ alkylene-CN, or C₂-C₃ heteroalkyl;
each R⁶ is independently halogen, —OR⁷, —NR⁴R⁵, C₁-C₃ alkyl, —CF₂H, —CF₃, C₃-C₆ cycloalkyl, or —CN;
each R⁷ is independently —H, C₁-C₃ alkyl, —CD₃, —CF₂H, —CF₃, or C₃-C₆ cycloalkyl; and
R⁸ is —H, —F, or —CH₃.

5. The compound of claim 1, or a pharmaceutically acceptable salt, solvate, hydrate, or co-crystal thereof, or a mixture of any of the foregoing, wherein:
each R¹ is independently —F, C₁-C₃ alkylene-NR^{4′}R^{5′}, C₁-C₃ alkylene-OH, or C₀-C₃ alkylene-CN,
wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl.

6. The compound of claim 1, or a pharmaceutically acceptable salt, solvate, hydrate, or co-crystal thereof, or a mixture of any of the foregoing, wherein:
R² is phenyl, 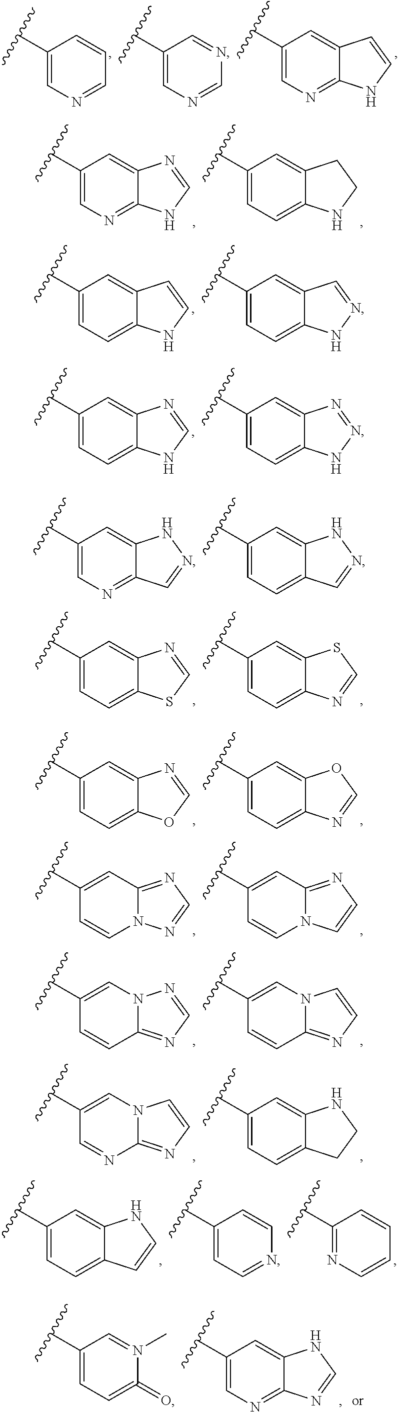 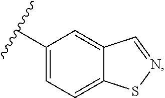 [SMILES: *c1ccc2sncc2c1]
each of which is optionally substituted with 1-5 R⁶ groups.

7. The compound of claim 1, or a pharmaceutically acceptable salt, solvate, hydrate, or co-crystal thereof, or a mixture of any of the foregoing, wherein:
each R⁶ is independently halogen, —OR⁷, —NR⁴R⁵, C₁-C₃ alkyl, —CF₃, or —CN,
wherein each R⁴ of R⁶ and each R⁵ of R⁶ are independently —H or C₁-C₃ alkyl; and
each R⁷ is independently —H, C₁-C₂ alkyl, —CD₃, C₁-C₂haloalkyl, or C₃ cycloalkyl.

8. The compound of claim 1, or a pharmaceutically acceptable salt, solvate, hydrate, or co-crystal thereof, or a mixture of any of the foregoing, wherein:
each R¹ is independently —F, C₁-C₃ alkylene-NR^{4′}R^{5′}, C₁-C₃ alkylene-OH, or C₀-C₃ alkylene-CN,
wherein each pair of R^{4′} and R^{5′} of R¹ taken together with the nitrogen atom to which they are attached independently form a 4-to-6-membered heterocyclic ring, wherein the heterocyclic ring optionally contains an additional 1-2 heteroatoms selected from the group consisting of N and O, and wherein each additional nitrogen atom, if present, is independently optionally substituted with C₁-C₃ alkyl;
R² is phenyl, 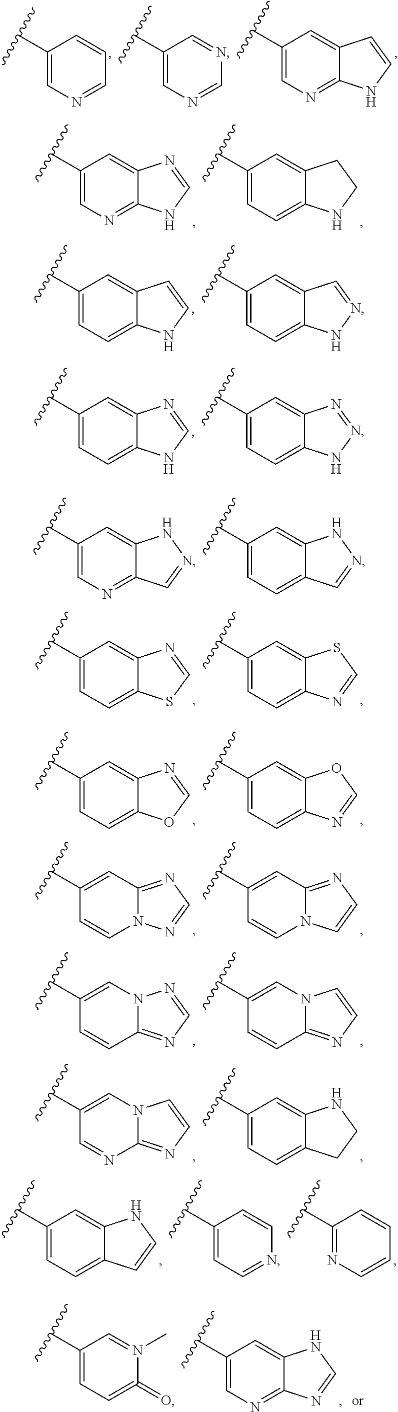 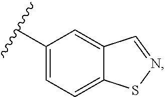 [SMILES: *c1ccc2sncc2c1]
each of which is optionally substituted with 1-5 R⁶ groups;
each R⁶ is independently halogen, —OR⁷, —NR⁴R⁵, C₁-C₃ alkyl, —CF₃, or —CN, wherein each R⁴ of R⁶ and each R⁵ of R⁶ are independently —H or C₁-C₃ alkyl; and
each R⁷ is independently —H, C₁-C₂ alkyl, —CD₃, C₁-C₂ haloalkyl, or C₃ cycloalkyl.

9. The compound of claim 1, or a pharmaceutically acceptable salt, solvate, hydrate, or co-crystal thereof, or a mixture of any of the foregoing, wherein:
each R¹ is independently F, 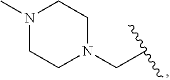 [SMILES: *CN1CCN(C)CC1]
—CH₂OH, —CH₂CH₂OH, —CN, or —CH₂CN.

10. The compound of claim 1, or a pharmaceutically acceptable salt, solvate, hydrate, or co-crystal thereof, or a mixture of any of the foregoing, wherein:
each R⁶ is independently —F, —Cl, —OH, —OCH₃, —OCH₂CH₃, —OCF₃, —OCF₂H, —OCH₂CF₃, —OCD₃, cyclopropyloxy, —NH₂, —NHCH₃, —N(CH₃)₂, —CH₃, —CF₃, or —CN.

11-19. (canceled)

20. A compound selected from the group consisting of 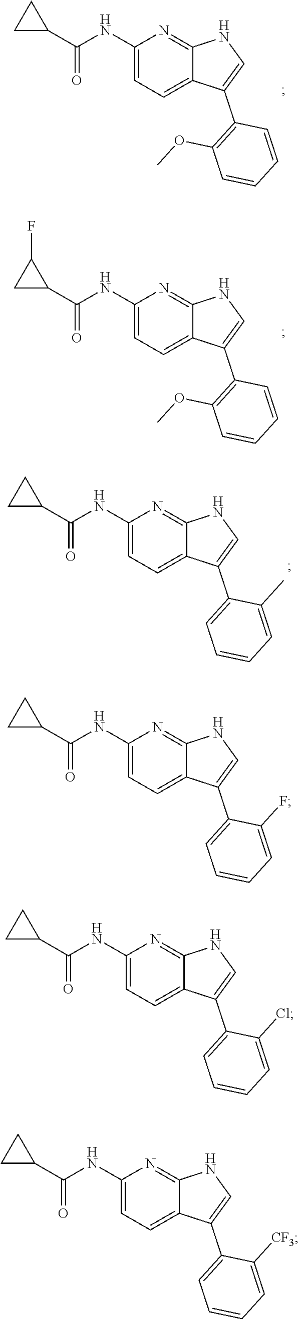 [SMILES: COc1ccccc1-c1c[nH]c2nc(NC(=O)C3CC3)ccc12 ; COc1ccccc1-c1c[nH]c2nc(NC(=O)C3CC3F)ccc12 ; Cc1ccccc1-c1c[nH]c2nc(NC(=O)C3CC3)ccc12 ; O=C(Nc1ccc2c(-c3ccccc3F)c[nH]c2n1)C1CC1 ; O=C(Nc1ccc2c(-c3ccccc3Cl)c[nH]c2n1)C1CC1 ; O=C(Nc1ccc2c(-c3ccccc3C(F)(F)F)c[nH]c2n1)C1CC1] 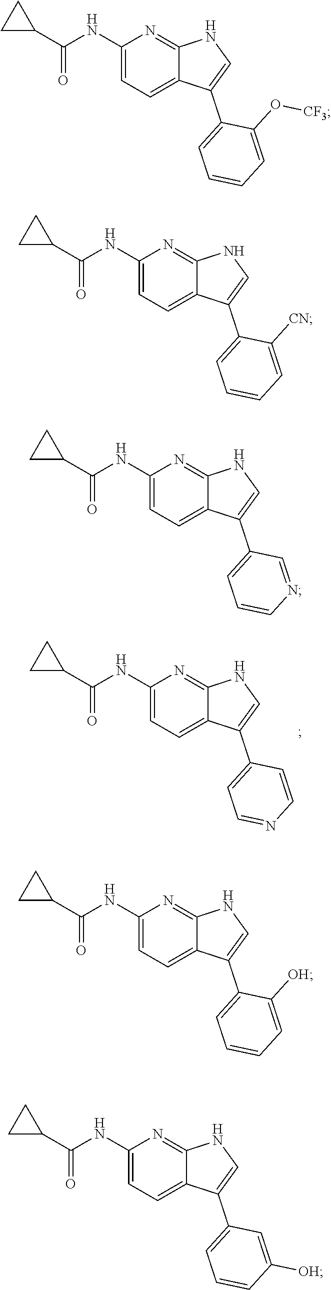 [SMILES: O=C(Nc1ccc2c(-c3ccccc3OC(F)(F)F)c[nH]c2n1)C1CC1 ; N#Cc1ccccc1-c1c[nH]c2nc(NC(=O)C3CC3)ccc12 ; O=C(Nc1ccc2c(-c3cccnc3)c[nH]c2n1)C1CC1 ; O=C(Nc1ccc2c(-c3ccncc3)c[nH]c2n1)C1CC1 ; O=C(Nc1ccc2c(-c3ccccc3O)c[nH]c2n1)C1CC1 ; O=C(Nc1ccc2c(-c3cccc(O)c3)c[nH]c2n1)C1CC1] 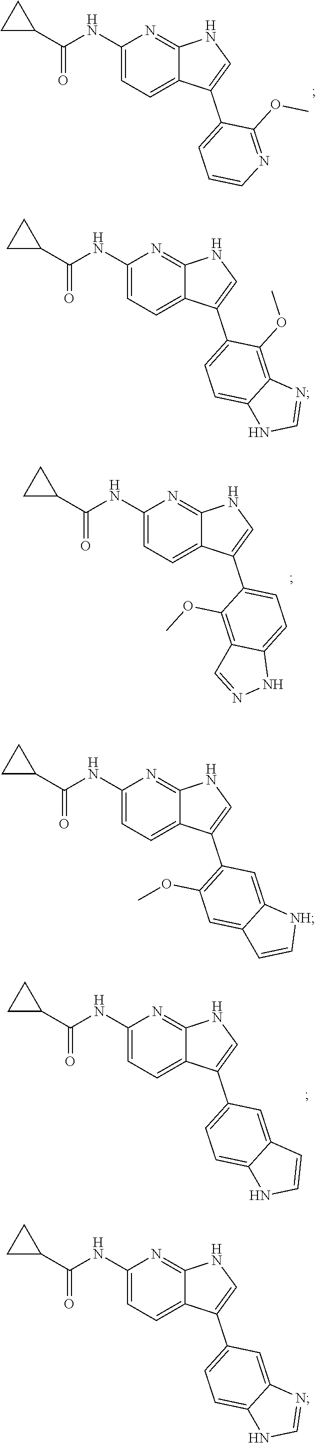 [SMILES: COc1ncccc1-c1c[nH]c2nc(NC(=O)C3CC3)ccc12 ; COc1c(-c2c[nH]c3nc(NC(=O)C4CC4)ccc23)ccc2[nH]cnc12 ; COc1c(-c2c[nH]c3nc(NC(=O)C4CC4)ccc23)ccc2[nH]ncc12 ; COc1cc2cc[nH]c2cc1-c1c[nH]c2nc(NC(=O)C3CC3)ccc12 ; O=C(Nc1ccc2c(-c3ccc4[nH]ccc4c3)c[nH]c2n1)C1CC1 ; O=C(Nc1ccc2c(-c3ccc4[nH]cnc4c3)c[nH]c2n1)C1CC1] 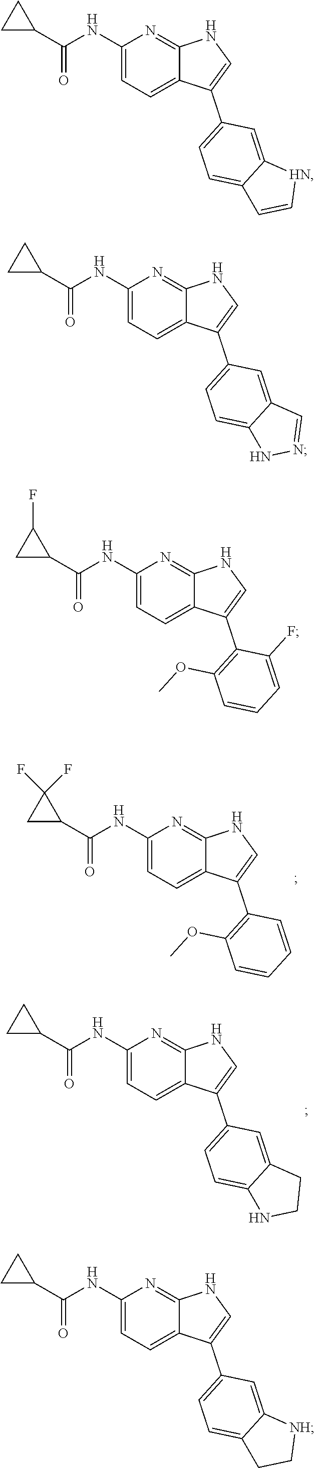 [SMILES: O=C(Nc1ccc2c(-c3ccc4cc[nH]c4c3)c[nH]c2n1)C1CC1 ; O=C(Nc1ccc2c(-c3ccc4[nH]ncc4c3)c[nH]c2n1)C1CC1 ; COc1cccc(F)c1-c1c[nH]c2nc(NC(=O)C3CC3F)ccc12 ; COc1ccccc1-c1c[nH]c2nc(NC(=O)C3CC3(F)F)ccc12 ; O=C(Nc1ccc2c(-c3ccc4c(c3)CCN4)c[nH]c2n1)C1CC1 ; O=C(Nc1ccc2c(-c3ccc4c(c3)NCC4)c[nH]c2n1)C1CC1] 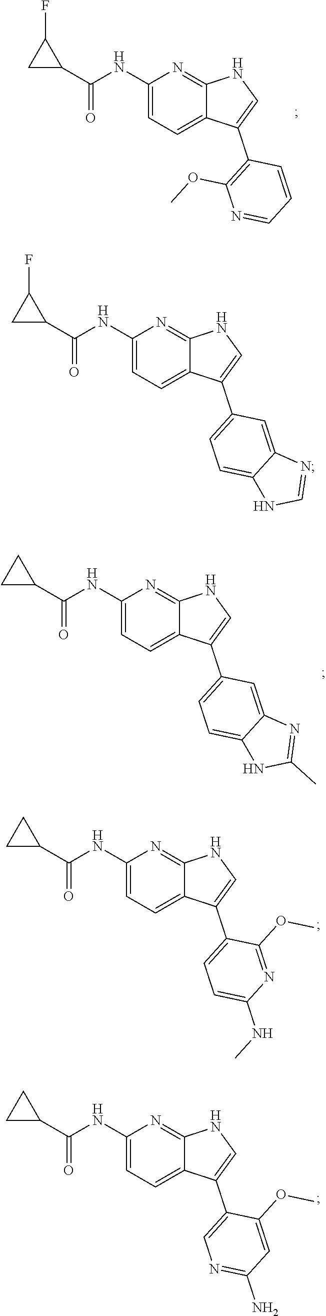 [SMILES: COc1ncccc1-c1c[nH]c2nc(NC(=O)C3CC3F)ccc12 ; O=C(Nc1ccc2c(-c3ccc4[nH]cnc4c3)c[nH]c2n1)C1CC1F ; Cc1nc2cc(-c3c[nH]c4nc(NC(=O)C5CC5)ccc34)ccc2[nH]1 ; CNc1ccc(-c2c[nH]c3nc(NC(=O)C4CC4)ccc23)c(OC)n1 ; COc1cc(N)ncc1-c1c[nH]c2nc(NC(=O)C3CC3)ccc12] 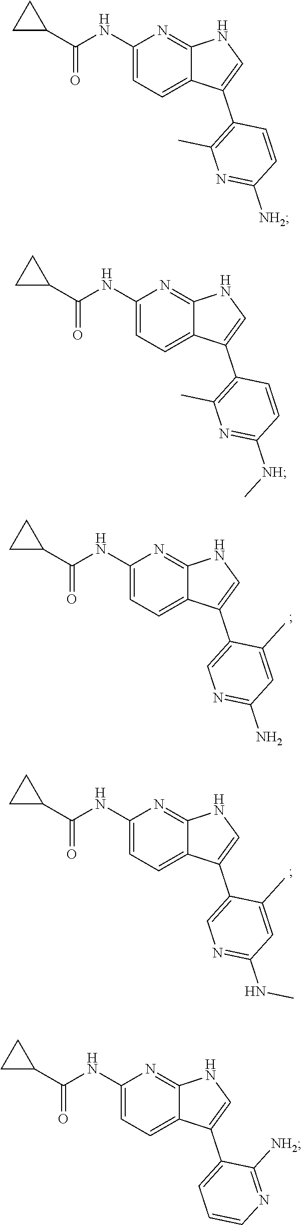 [SMILES: Cc1nc(N)ccc1-c1c[nH]c2nc(NC(=O)C3CC3)ccc12 ; CNc1ccc(-c2c[nH]c3nc(NC(=O)C4CC4)ccc23)c(C)n1 ; Cc1cc(N)ncc1-c1c[nH]c2nc(NC(=O)C3CC3)ccc12 ; CNc1cc(C)c(-c2c[nH]c3nc(NC(=O)C4CC4)ccc23)cn1 ; Nc1ncccc1-c1c[nH]c2nc(NC(=O)C3CC3)ccc12] 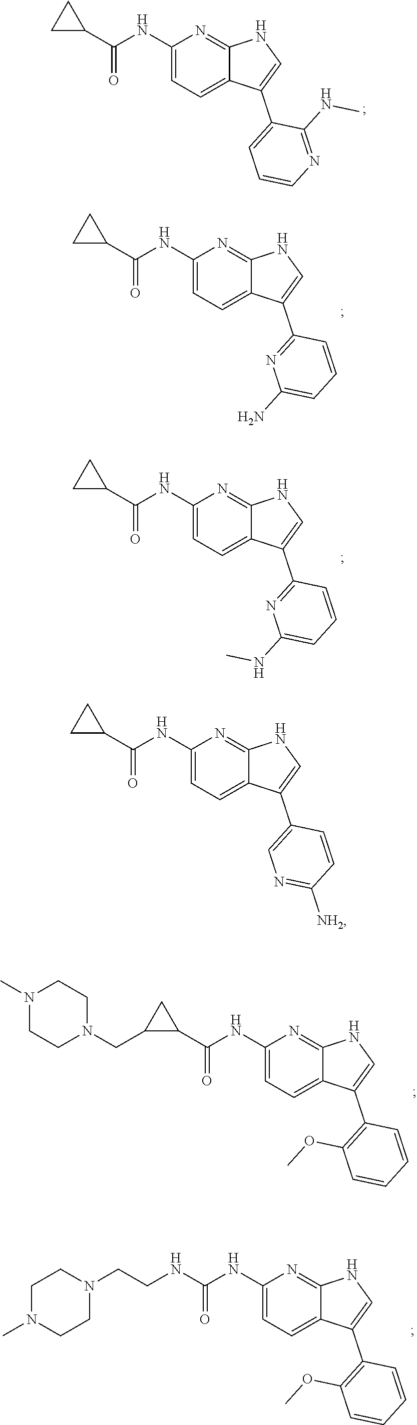 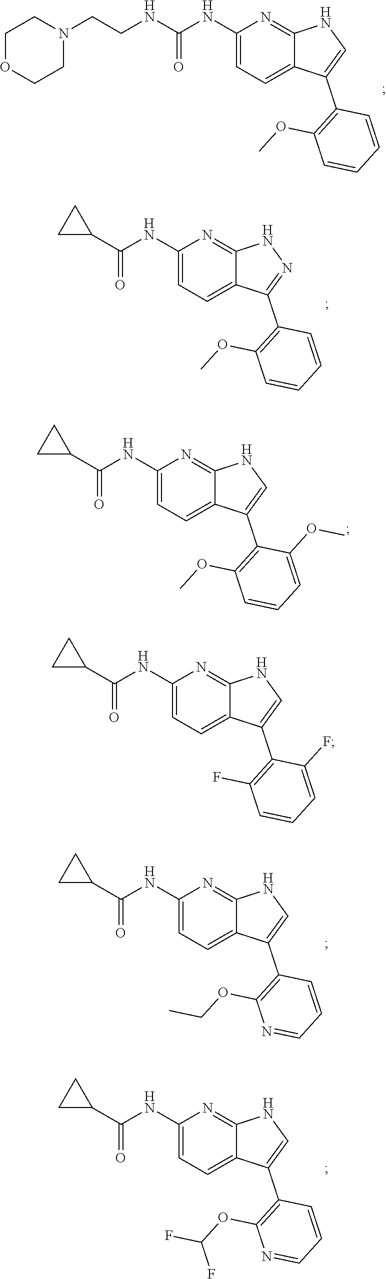 [SMILES: COc1ccccc1-c1c[nH]c2nc(NC(=O)NCCN3CCOCC3)ccc12 ; COc1ccccc1-c1n[nH]c2nc(NC(=O)C3CC3)ccc12 ; COc1cccc(OC)c1-c1c[nH]c2nc(NC(=O)C3CC3)ccc12 ; O=C(Nc1ccc2c(-c3c(F)cccc3F)c[nH]c2n1)C1CC1 ; CCOc1ncccc1-c1c[nH]c2nc(NC(=O)C3CC3)ccc12 ; O=C(Nc1ccc2c(-c3cccnc3OC(F)F)c[nH]c2n1)C1CC1] 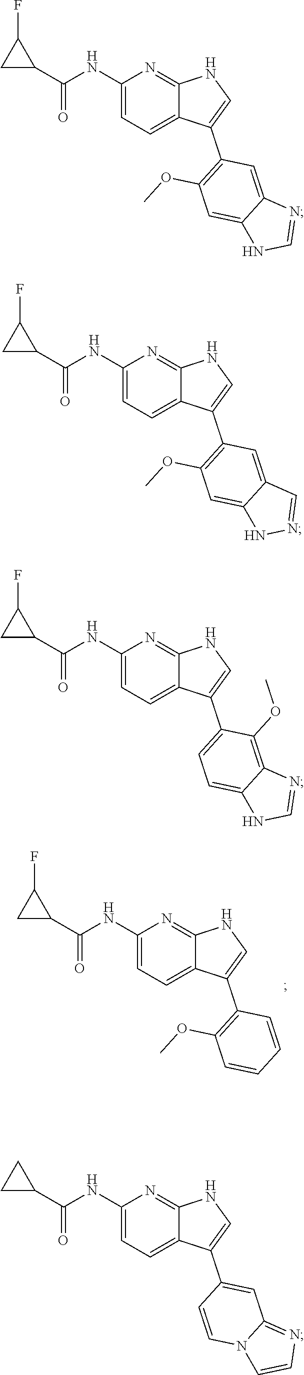 [SMILES: COc1cc2[nH]cnc2cc1-c1c[nH]c2nc(NC(=O)C3CC3F)ccc12 ; COc1cc2[nH]ncc2cc1-c1c[nH]c2nc(NC(=O)C3CC3F)ccc12 ; COc1c(-c2c[nH]c3nc(NC(=O)C4CC4F)ccc23)ccc2[nH]cnc12 ; COc1ccccc1-c1c[nH]c2nc(NC(=O)C3CC3F)ccc12 ; O=C(Nc1ccc2c(-c3ccn4ccnc4c3)c[nH]c2n1)C1CC1] 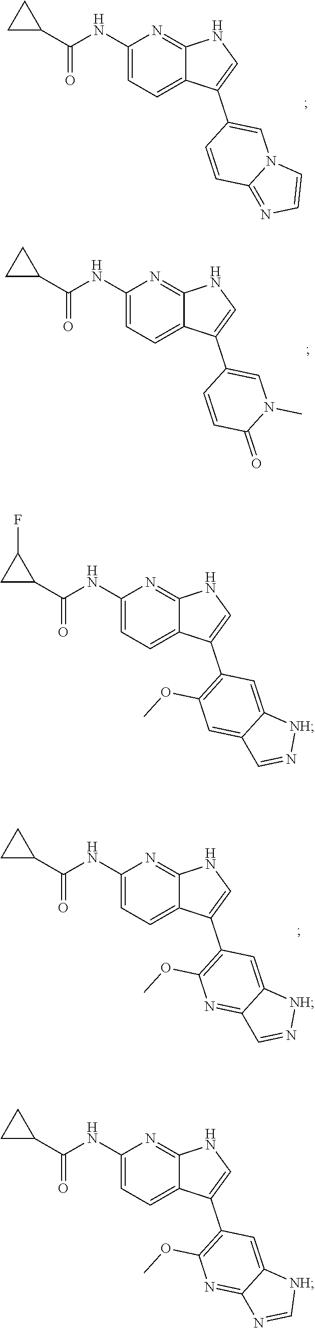 [SMILES: O=C(Nc1ccc2c(-c3ccc4nccn4c3)c[nH]c2n1)C1CC1 ; Cn1cc(-c2c[nH]c3nc(NC(=O)C4CC4)ccc23)ccc1=O ; COc1cc2cn[nH]c2cc1-c1c[nH]c2nc(NC(=O)C3CC3F)ccc12 ; COc1nc2cn[nH]c2cc1-c1c[nH]c2nc(NC(=O)C3CC3)ccc12 ; COc1nc2nc[nH]c2cc1-c1c[nH]c2nc(NC(=O)C3CC3)ccc12] 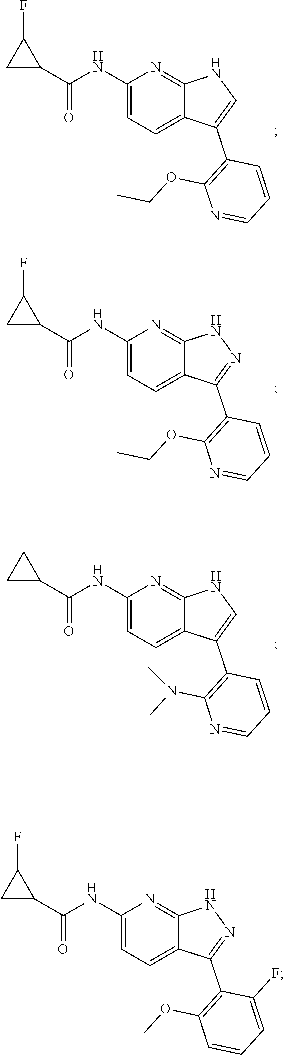 [SMILES: CCOc1ncccc1-c1c[nH]c2nc(NC(=O)C3CC3F)ccc12 ; CCOc1ncccc1-c1n[nH]c2nc(NC(=O)C3CC3F)ccc12 ; CN(C)c1ncccc1-c1c[nH]c2nc(NC(=O)C3CC3)ccc12 ; COc1cccc(F)c1-c1n[nH]c2nc(NC(=O)C3CC3F)ccc12] 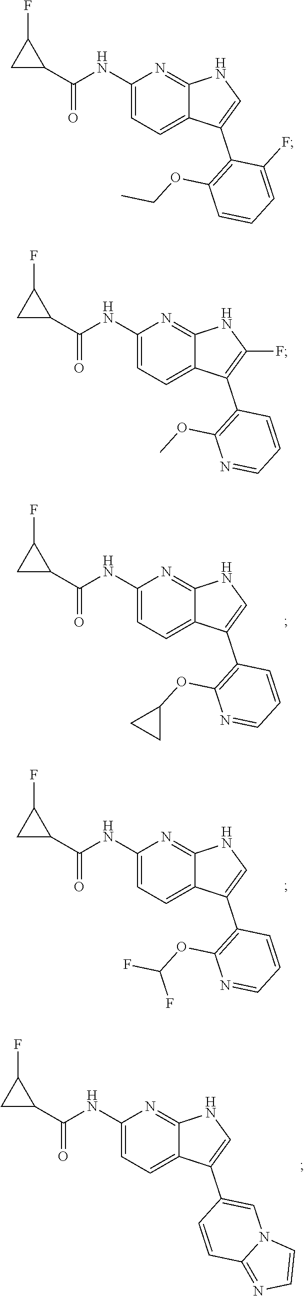 [SMILES: CCOc1cccc(F)c1-c1c[nH]c2nc(NC(=O)C3CC3F)ccc12 ; COc1ncccc1-c1c(F)[nH]c2nc(NC(=O)C3CC3F)ccc12 ; O=C(Nc1ccc2c(-c3cccnc3OC3CC3)c[nH]c2n1)C1CC1F ; O=C(Nc1ccc2c(-c3cccnc3OC(F)F)c[nH]c2n1)C1CC1F ; O=C(Nc1ccc2c(-c3ccc4nccn4c3)c[nH]c2n1)C1CC1F] 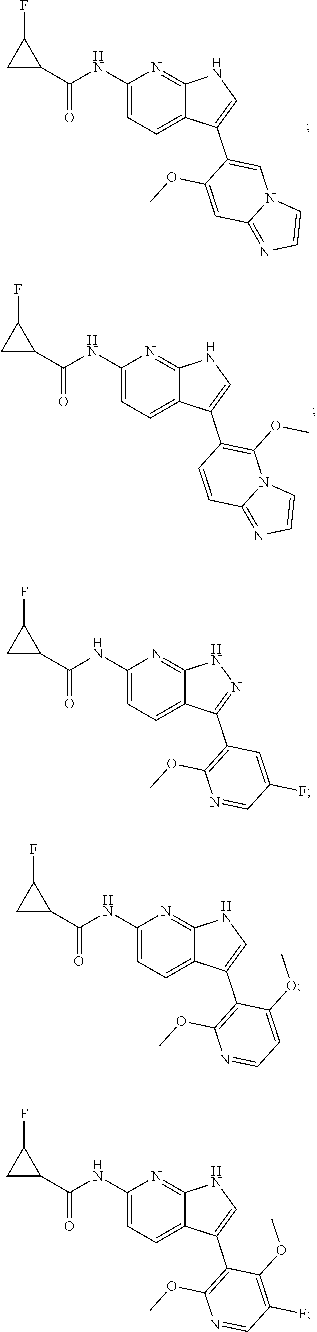 [SMILES: COc1cc2nccn2cc1-c1c[nH]c2nc(NC(=O)C3CC3F)ccc12 ; COc1c(-c2c[nH]c3nc(NC(=O)C4CC4F)ccc23)ccc2nccn12 ; COc1ncc(F)cc1-c1n[nH]c2nc(NC(=O)C3CC3F)ccc12 ; COc1ccnc(OC)c1-c1c[nH]c2nc(NC(=O)C3CC3F)ccc12 ; COc1ncc(F)c(OC)c1-c1c[nH]c2nc(NC(=O)C3CC3F)ccc12] 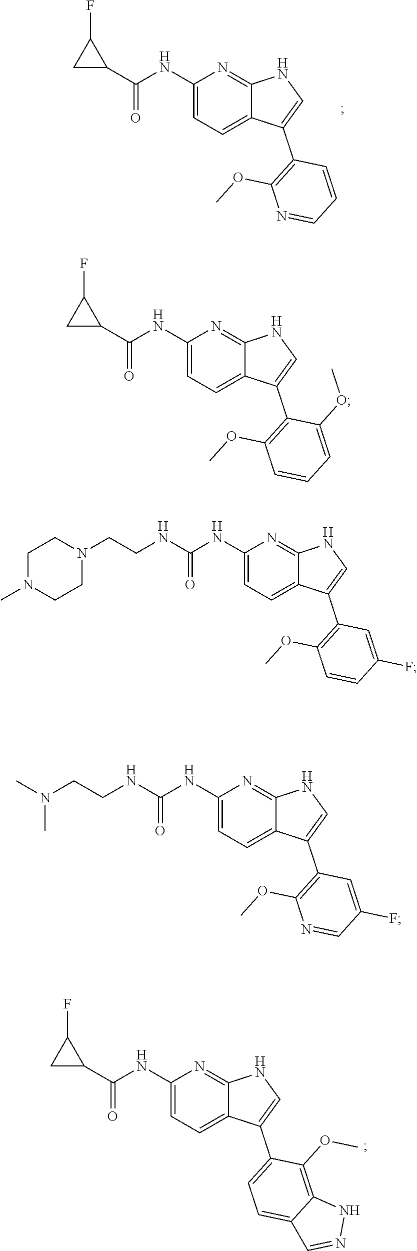 [SMILES: COc1ncccc1-c1c[nH]c2nc(NC(=O)C3CC3F)ccc12 ; COc1cccc(OC)c1-c1c[nH]c2nc(NC(=O)C3CC3F)ccc12 ; COc1ccc(F)cc1-c1c[nH]c2nc(NC(=O)NCCN3CCN(C)CC3)ccc12 ; COc1ncc(F)cc1-c1c[nH]c2nc(NC(=O)NCCN(C)C)ccc12 ; COc1c(-c2c[nH]c3nc(NC(=O)C4CC4F)ccc23)ccc2cn[nH]c12] 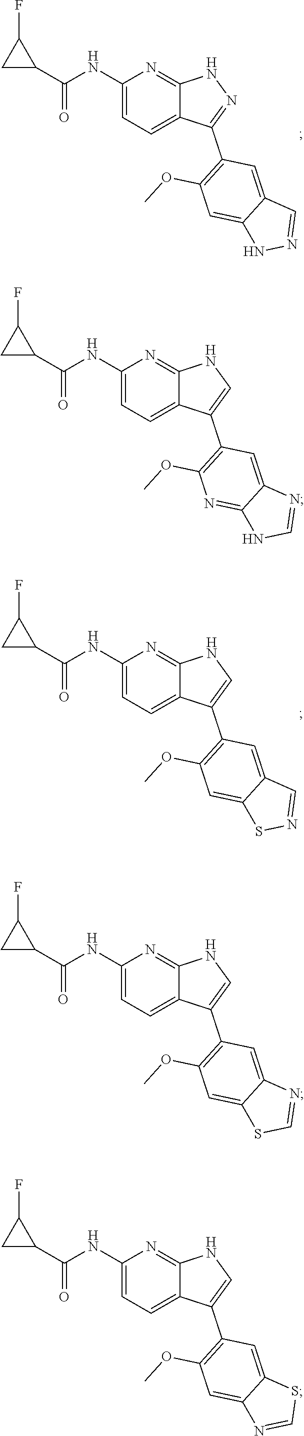 [SMILES: COc1cc2[nH]ncc2cc1-c1n[nH]c2nc(NC(=O)C3CC3F)ccc12 ; COc1nc2[nH]cnc2cc1-c1c[nH]c2nc(NC(=O)C3CC3F)ccc12 ; COc1cc2sncc2cc1-c1c[nH]c2nc(NC(=O)C3CC3F)ccc12 ; COc1cc2scnc2cc1-c1c[nH]c2nc(NC(=O)C3CC3F)ccc12 ; COc1cc2ncsc2cc1-c1c[nH]c2nc(NC(=O)C3CC3F)ccc12] 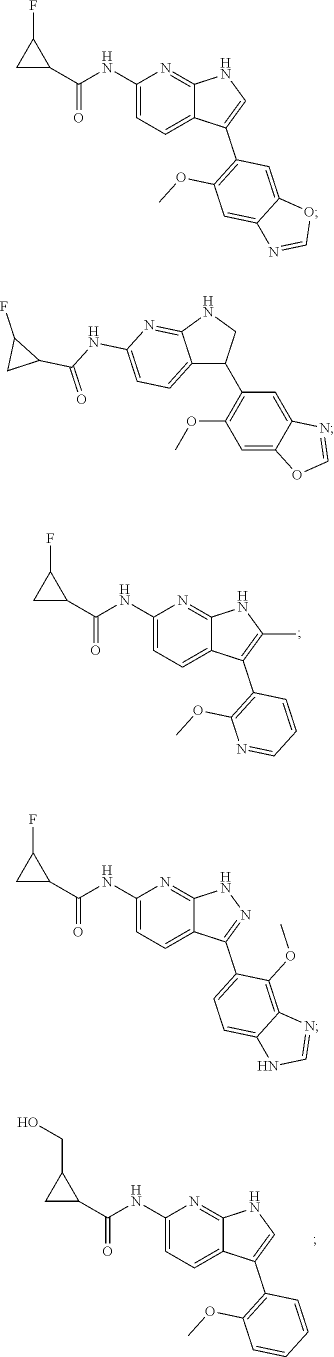 [SMILES: COc1cc2ncoc2cc1-c1c[nH]c2nc(NC(=O)C3CC3F)ccc12 ; COc1cc2ocnc2cc1C1CNc2nc(NC(=O)C3CC3F)ccc21 ; COc1ncccc1-c1c(C)[nH]c2nc(NC(=O)C3CC3F)ccc12 ; COc1c(-c2n[nH]c3nc(NC(=O)C4CC4F)ccc23)ccc2[nH]cnc12 ; COc1ccccc1-c1c[nH]c2nc(NC(=O)C3CC3CO)ccc12] 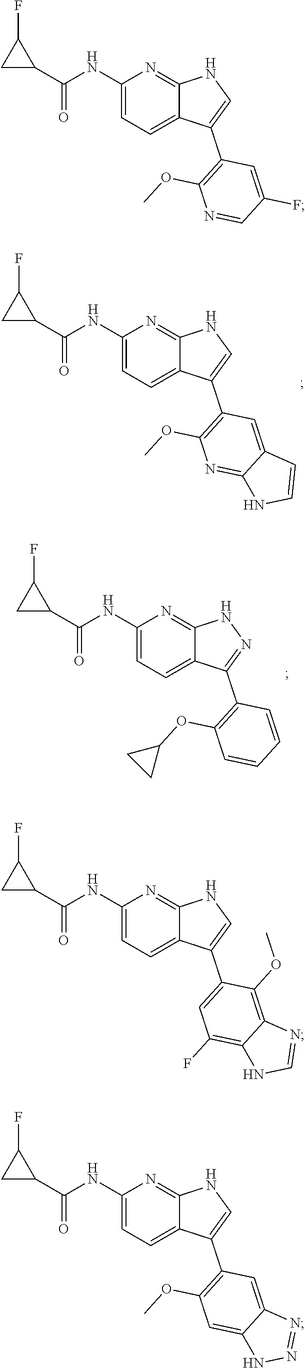 [SMILES: COc1ncc(F)cc1-c1c[nH]c2nc(NC(=O)C3CC3F)ccc12 ; COc1nc2[nH]ccc2cc1-c1c[nH]c2nc(NC(=O)C3CC3F)ccc12 ; O=C(Nc1ccc2c(-c3ccccc3OC3CC3)n[nH]c2n1)C1CC1F ; COc1c(-c2c[nH]c3nc(NC(=O)C4CC4F)ccc23)cc(F)c2[nH]cnc12 ; COc1cc2[nH]nnc2cc1-c1c[nH]c2nc(NC(=O)C3CC3F)ccc12] 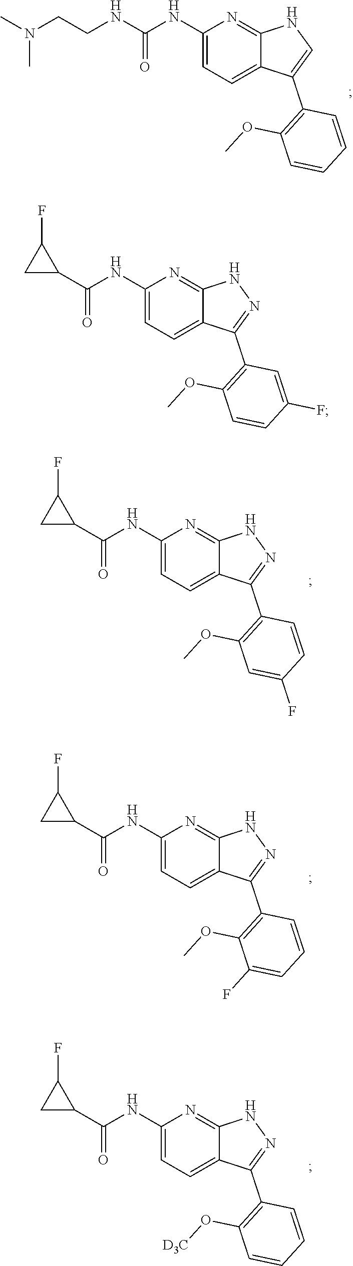 [SMILES: COc1ccccc1-c1c[nH]c2nc(NC(=O)NCCN(C)C)ccc12 ; COc1ccc(F)cc1-c1n[nH]c2nc(NC(=O)C3CC3F)ccc12 ; COc1cc(F)ccc1-c1n[nH]c2nc(NC(=O)C3CC3F)ccc12 ; COc1c(F)cccc1-c1n[nH]c2nc(NC(=O)C3CC3F)ccc12 ; [2H]C([2H])([2H])Oc1ccccc1-c1n[nH]c2nc(NC(=O)C3CC3F)ccc12] 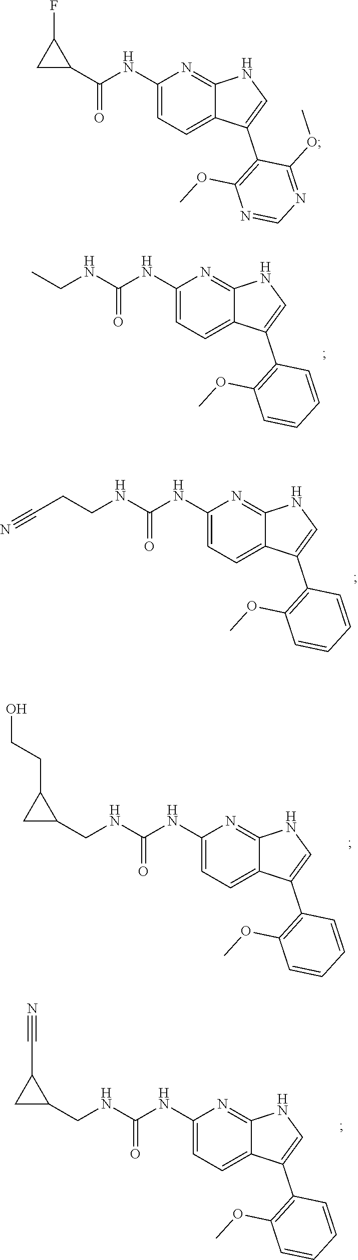 [SMILES: COc1ncnc(OC)c1-c1c[nH]c2nc(NC(=O)C3CC3F)ccc12 ; CCNC(=O)Nc1ccc2c(-c3ccccc3OC)c[nH]c2n1 ; COc1ccccc1-c1c[nH]c2nc(NC(=O)NCCC#N)ccc12 ; COc1ccccc1-c1c[nH]c2nc(NC(=O)NCC3CC3CCO)ccc12 ; COc1ccccc1-c1c[nH]c2nc(NC(=O)NCC3CC3C#N)ccc12] 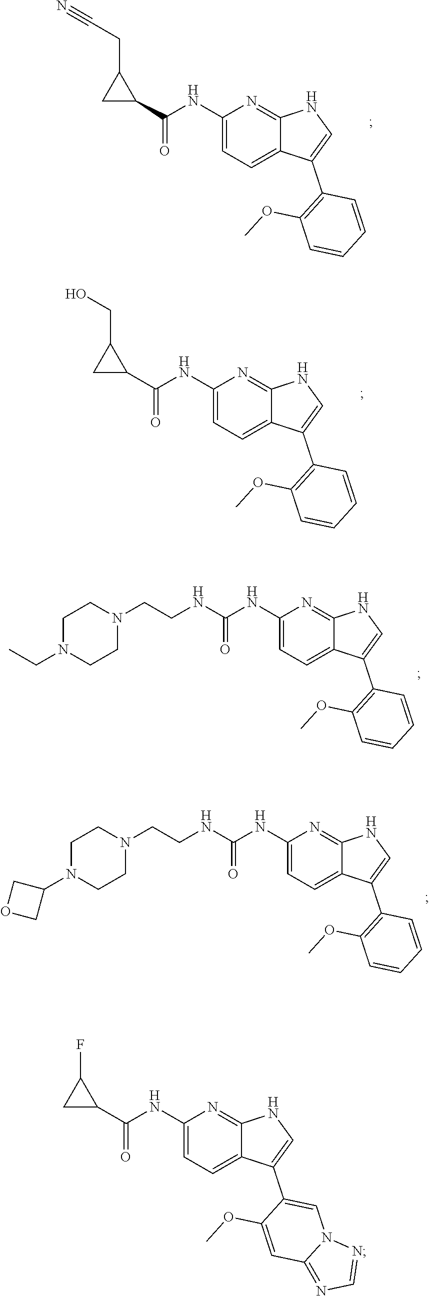 [SMILES: COc1ccccc1-c1c[nH]c2nc(NC(=O)[C@H]3CC3CC#N)ccc12 ; COc1ccccc1-c1c[nH]c2nc(NC(=O)C3CC3CO)ccc12 ; CCN1CCN(CCNC(=O)Nc2ccc3c(-c4ccccc4OC)c[nH]c3n2)CC1 ; COc1ccccc1-c1c[nH]c2nc(NC(=O)NCCN3CCN(C4COC4)CC3)ccc12 ; COc1cc2ncnn2cc1-c1c[nH]c2nc(NC(=O)C3CC3F)ccc12] 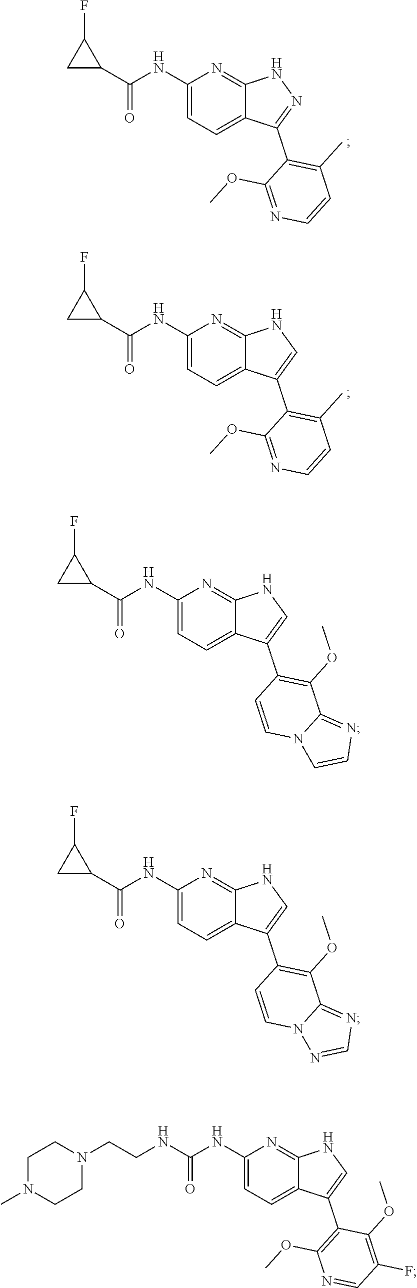 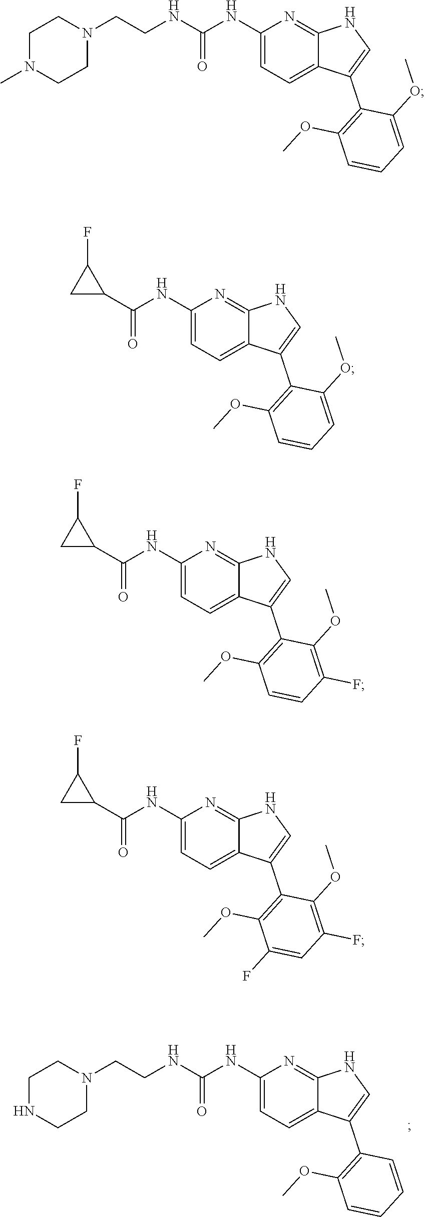 [SMILES: COc1cccc(OC)c1-c1c[nH]c2nc(NC(=O)NCCN3CCN(C)CC3)ccc12 ; COc1cccc(OC)c1-c1c[nH]c2nc(NC(=O)C3CC3F)ccc12 ; COc1ccc(F)c(OC)c1-c1c[nH]c2nc(NC(=O)C3CC3F)ccc12 ; COc1c(F)cc(F)c(OC)c1-c1c[nH]c2nc(NC(=O)C3CC3F)ccc12 ; COc1ccccc1-c1c[nH]c2nc(NC(=O)NCCN3CCNCC3)ccc12] 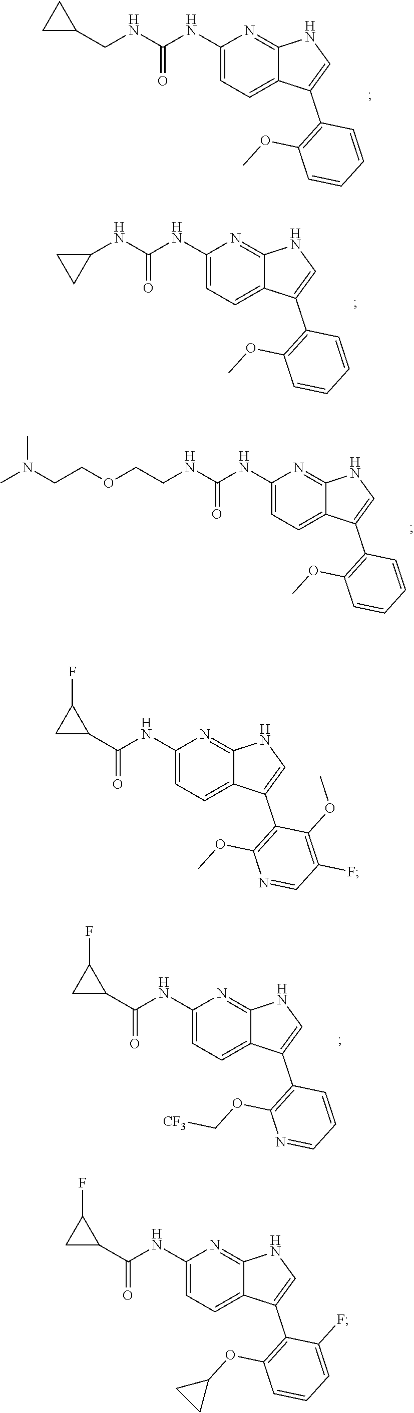 [SMILES: COc1ccccc1-c1c[nH]c2nc(NC(=O)NCC3CC3)ccc12 ; COc1ccccc1-c1c[nH]c2nc(NC(=O)NC3CC3)ccc12 ; COc1ccccc1-c1c[nH]c2nc(NC(=O)NCCOCCN(C)C)ccc12 ; COc1ncc(F)c(OC)c1-c1c[nH]c2nc(NC(=O)C3CC3F)ccc12 ; O=C(Nc1ccc2c(-c3cccnc3OCC(F)(F)F)c[nH]c2n1)C1CC1F ; O=C(Nc1ccc2c(-c3c(F)cccc3OC3CC3)c[nH]c2n1)C1CC1F] 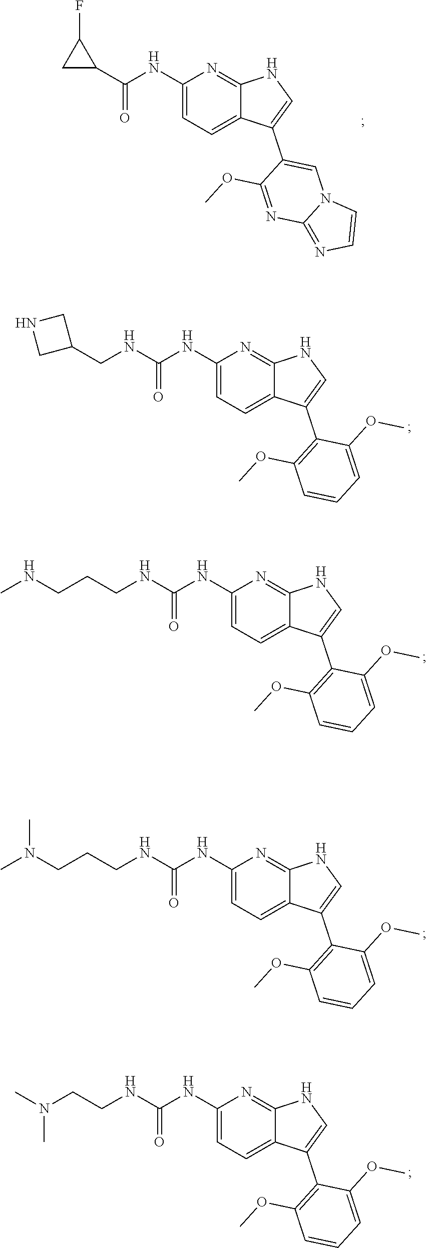 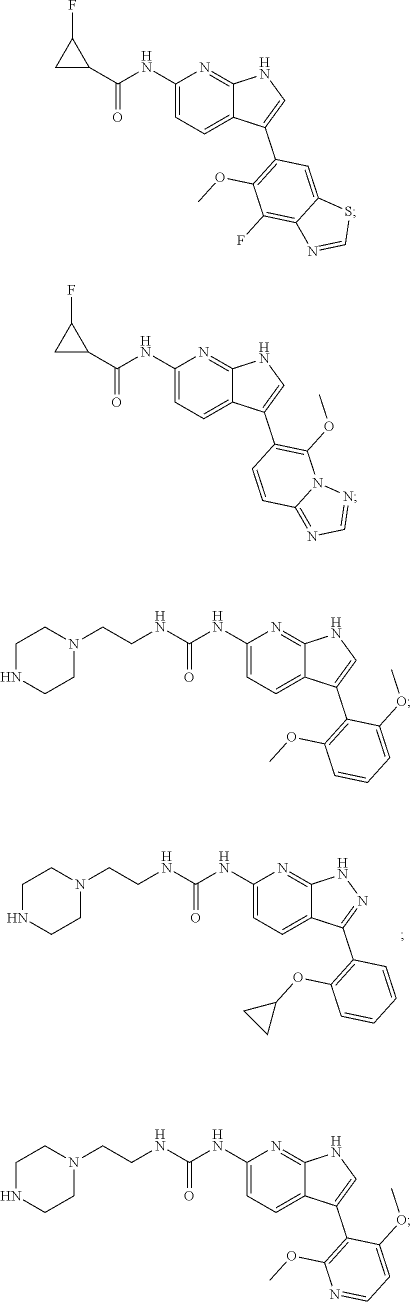 [SMILES: COc1c(-c2c[nH]c3nc(NC(=O)C4CC4F)ccc23)cc2scnc2c1F ; COc1c(-c2c[nH]c3nc(NC(=O)C4CC4F)ccc23)ccc2ncnn12 ; COc1cccc(OC)c1-c1c[nH]c2nc(NC(=O)NCCN3CCNCC3)ccc12 ; O=C(NCCN1CCNCC1)Nc1ccc2c(-c3ccccc3OC3CC3)n[nH]c2n1 ; COc1ccnc(OC)c1-c1c[nH]c2nc(NC(=O)NCCN3CCNCC3)ccc12] 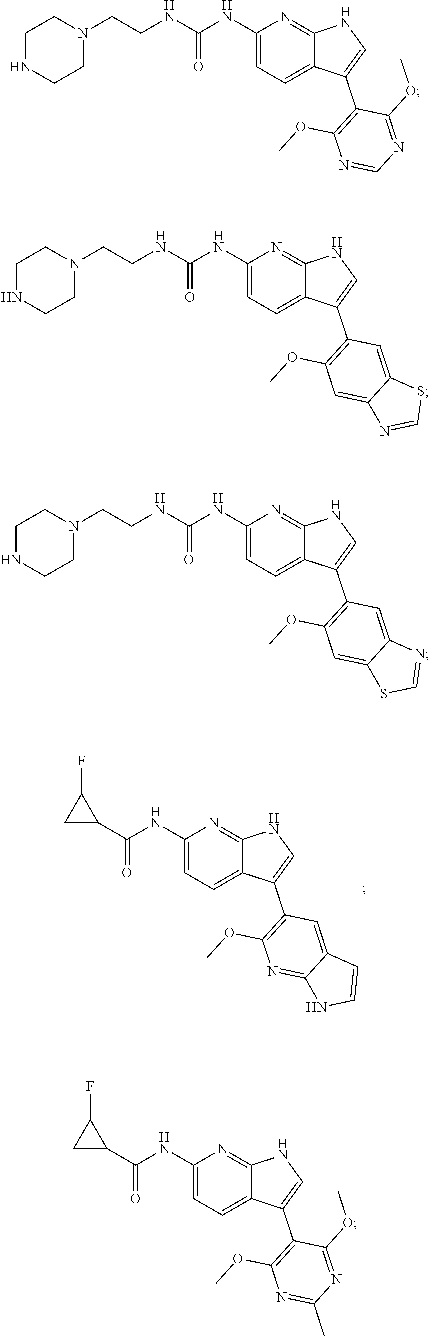 [SMILES: COc1ncnc(OC)c1-c1c[nH]c2nc(NC(=O)NCCN3CCNCC3)ccc12 ; COc1cc2ncsc2cc1-c1c[nH]c2nc(NC(=O)NCCN3CCNCC3)ccc12 ; COc1cc2scnc2cc1-c1c[nH]c2nc(NC(=O)NCCN3CCNCC3)ccc12 ; COc1nc2[nH]ccc2cc1-c1c[nH]c2nc(NC(=O)C3CC3F)ccc12 ; COc1nc(C)nc(OC)c1-c1c[nH]c2nc(NC(=O)C3CC3F)ccc12] 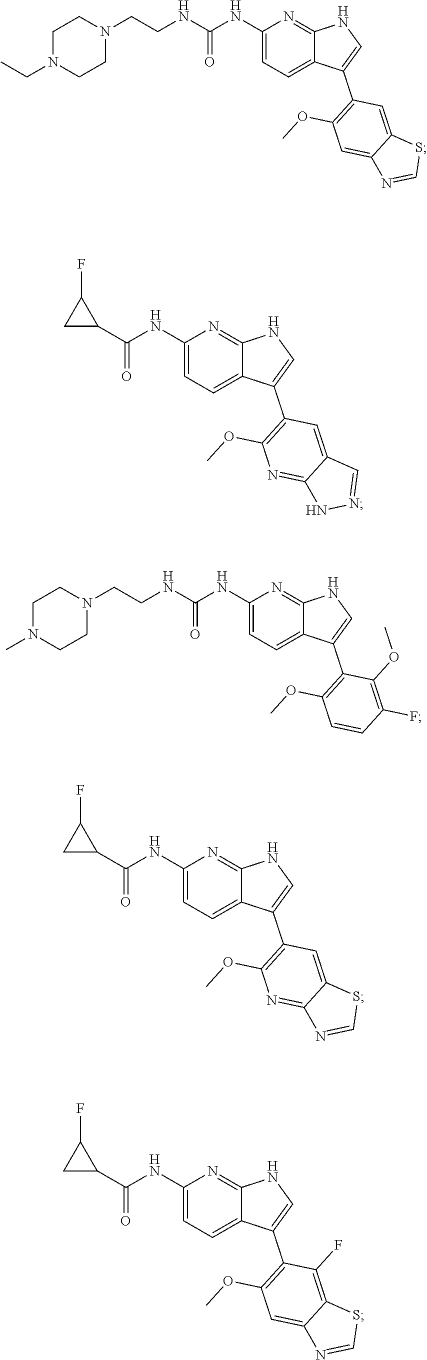 [SMILES: CCN1CCN(CCNC(=O)Nc2ccc3c(-c4cc5scnc5cc4OC)c[nH]c3n2)CC1 ; COc1nc2[nH]ncc2cc1-c1c[nH]c2nc(NC(=O)C3CC3F)ccc12 ; COc1ccc(F)c(OC)c1-c1c[nH]c2nc(NC(=O)NCCN3CCN(C)CC3)ccc12 ; COc1nc2ncsc2cc1-c1c[nH]c2nc(NC(=O)C3CC3F)ccc12 ; COc1cc2ncsc2c(F)c1-c1c[nH]c2nc(NC(=O)C3CC3F)ccc12] 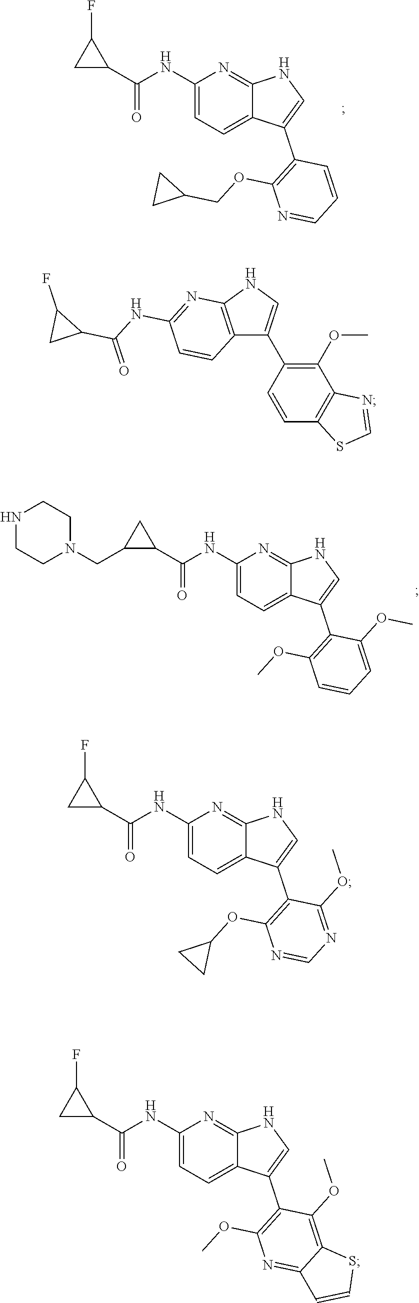 [SMILES: O=C(Nc1ccc2c(-c3cccnc3OCC3CC3)c[nH]c2n1)C1CC1F ; COc1c(-c2c[nH]c3nc(NC(=O)C4CC4F)ccc23)ccc2scnc12 ; COc1cccc(OC)c1-c1c[nH]c2nc(NC(=O)C3CC3CN3CCNCC3)ccc12 ; COc1ncnc(OC2CC2)c1-c1c[nH]c2nc(NC(=O)C3CC3F)ccc12 ; COc1nc2ccsc2c(OC)c1-c1c[nH]c2nc(NC(=O)C3CC3F)ccc12] 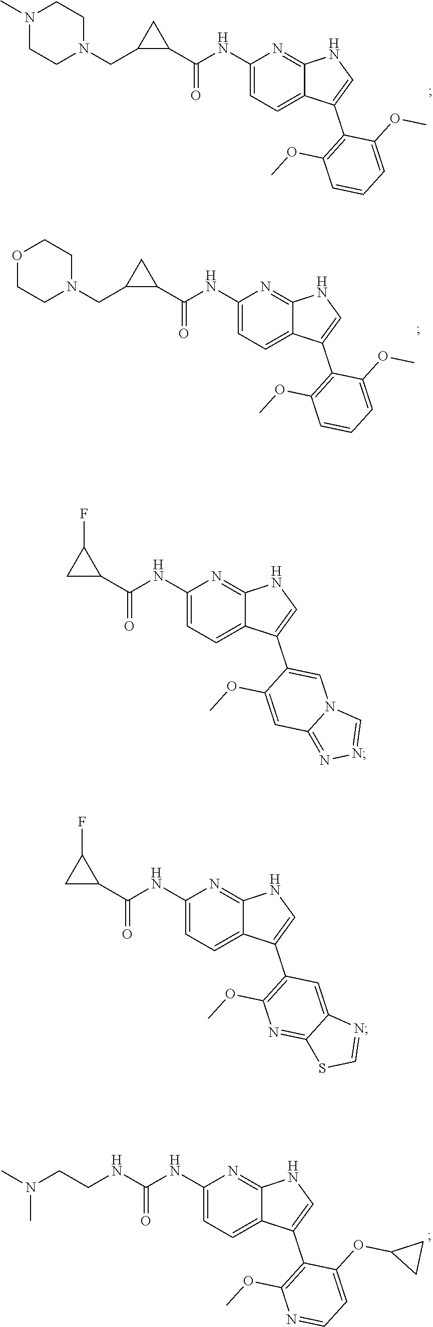 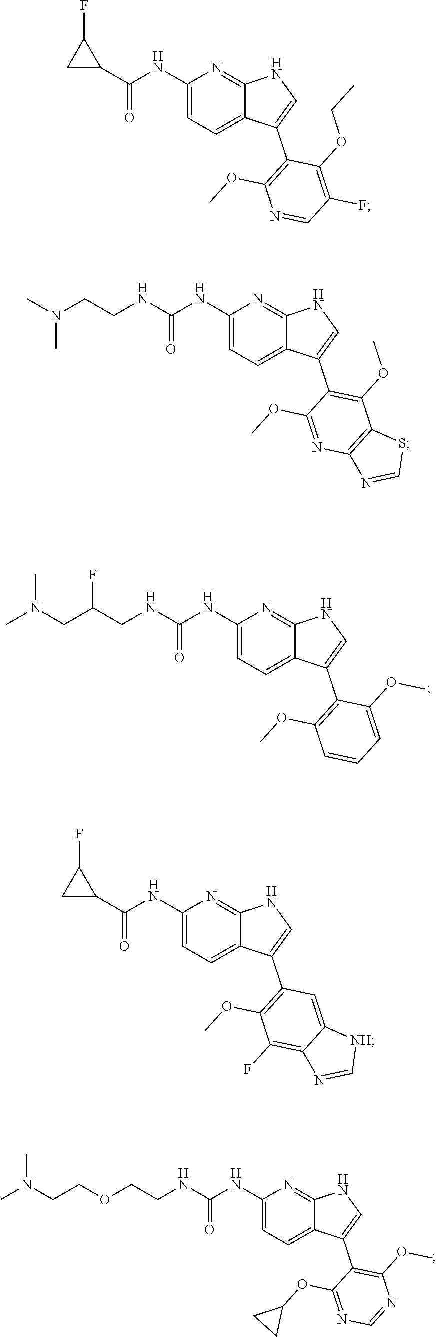 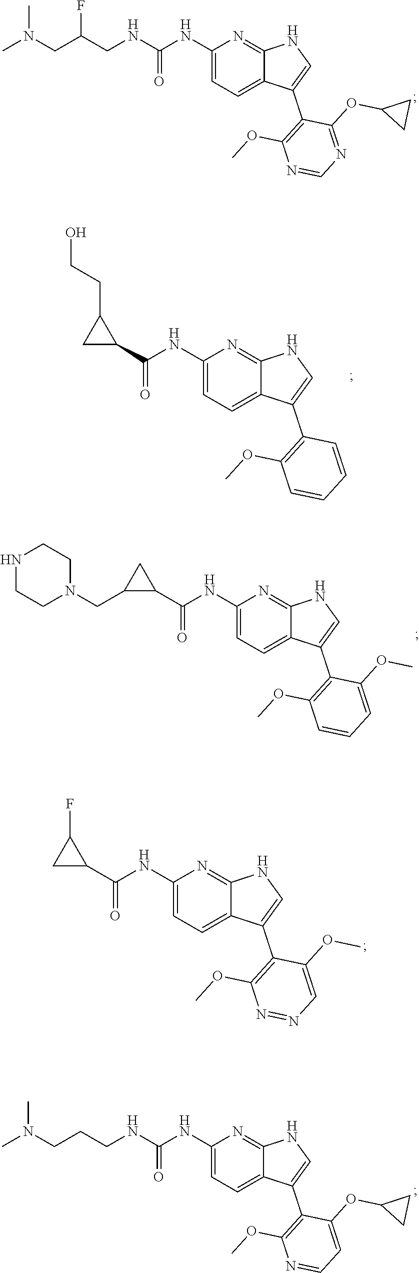 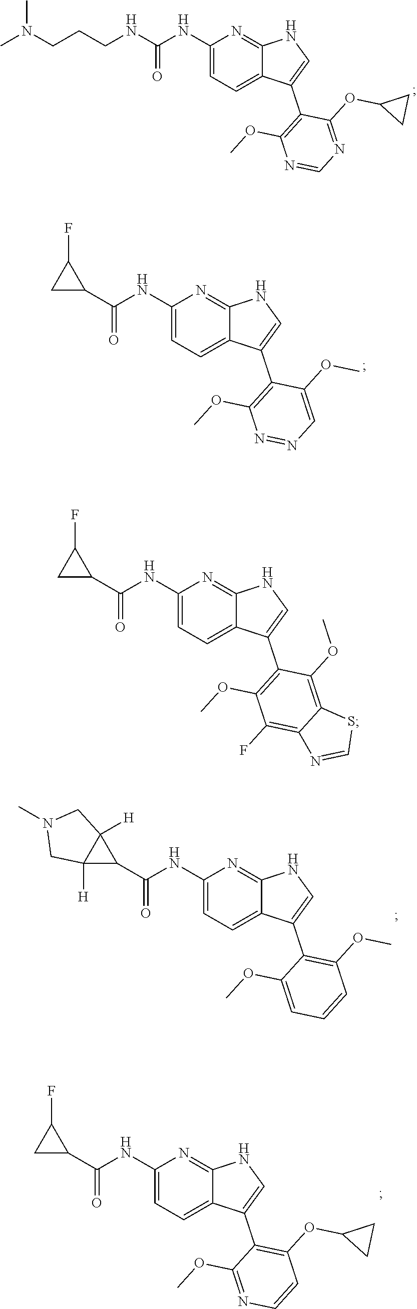 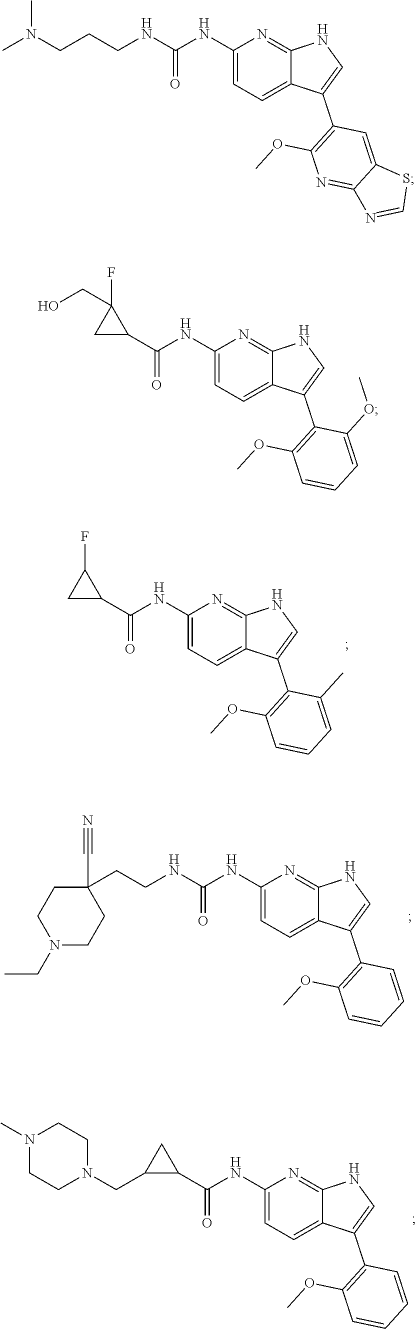 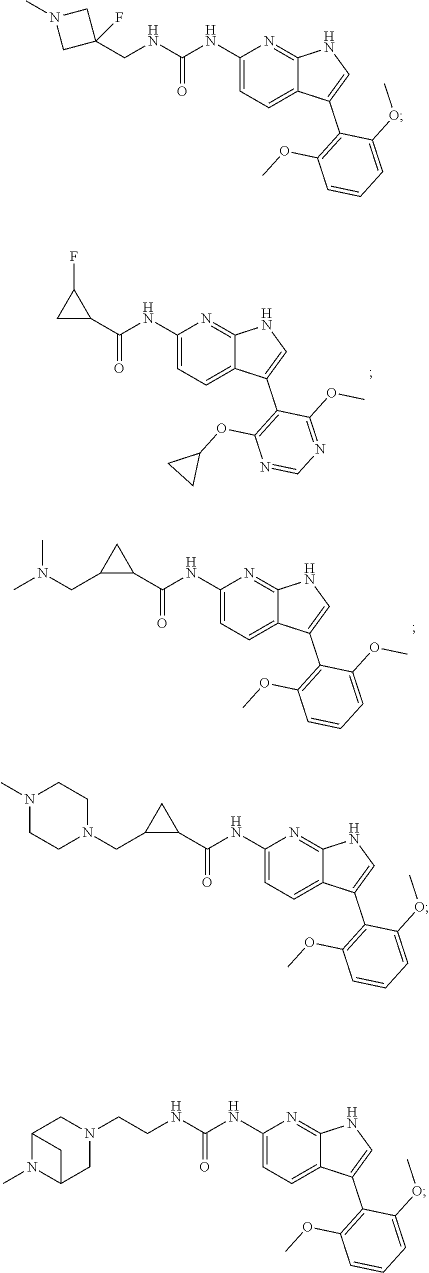 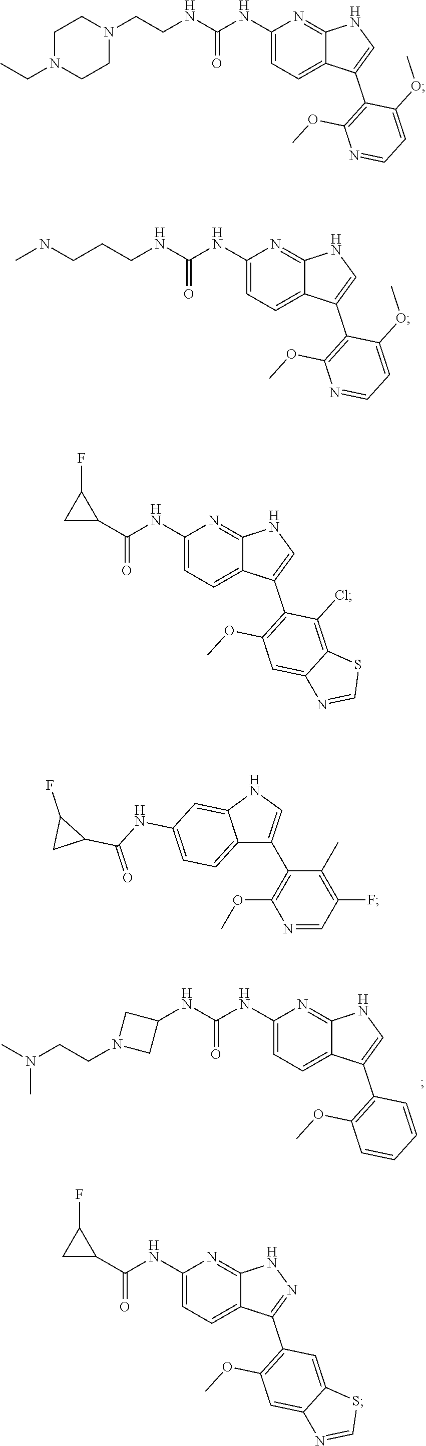 [SMILES: CCN1CCN(CCNC(=O)Nc2ccc3c(-c4c(OC)ccnc4OC)c[nH]c3n2)CC1 ; CNCCCNC(=O)Nc1ccc2c(-c3c(OC)ccnc3OC)c[nH]c2n1 ; COc1cc2ncsc2c(Cl)c1-c1c[nH]c2nc(NC(=O)C3CC3F)ccc12 ; COc1ncc(F)c(C)c1-c1c[nH]c2cc(NC(=O)C3CC3F)ccc12 ; COc1ccccc1-c1c[nH]c2nc(NC(=O)NC3CN(CCN(C)C)C3)ccc12 ; COc1cc2ncsc2cc1-c1n[nH]c2nc(NC(=O)C3CC3F)ccc12] 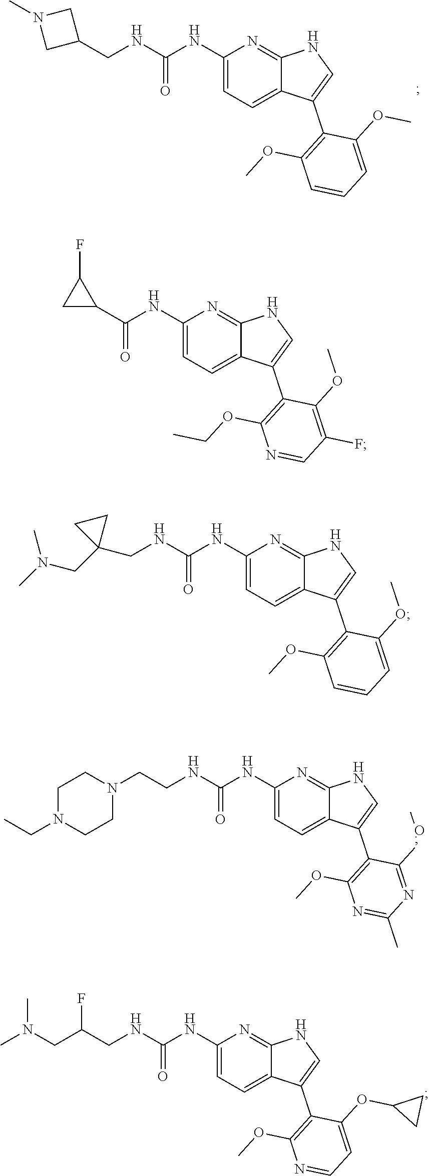 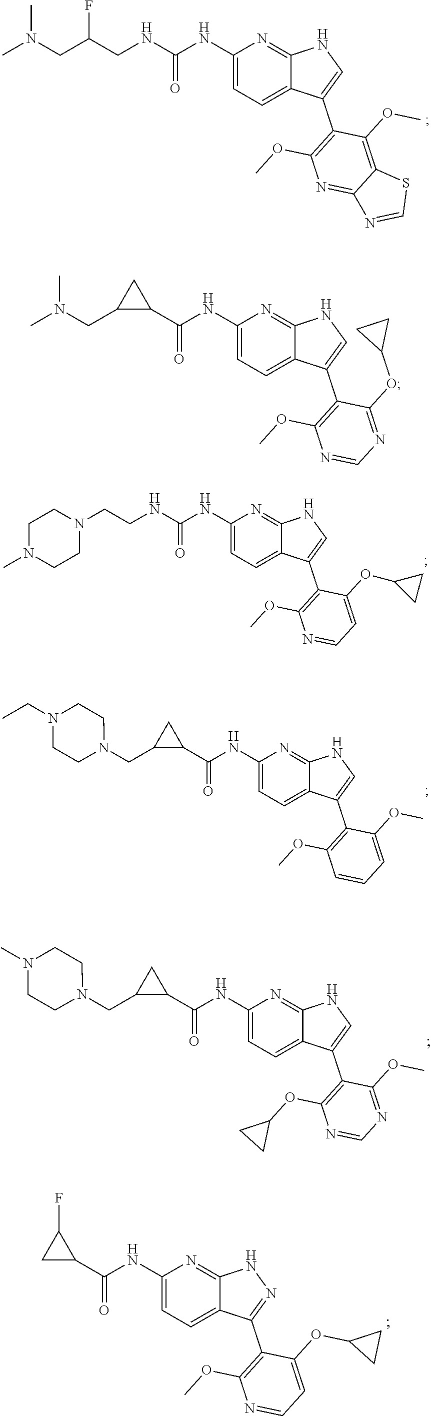 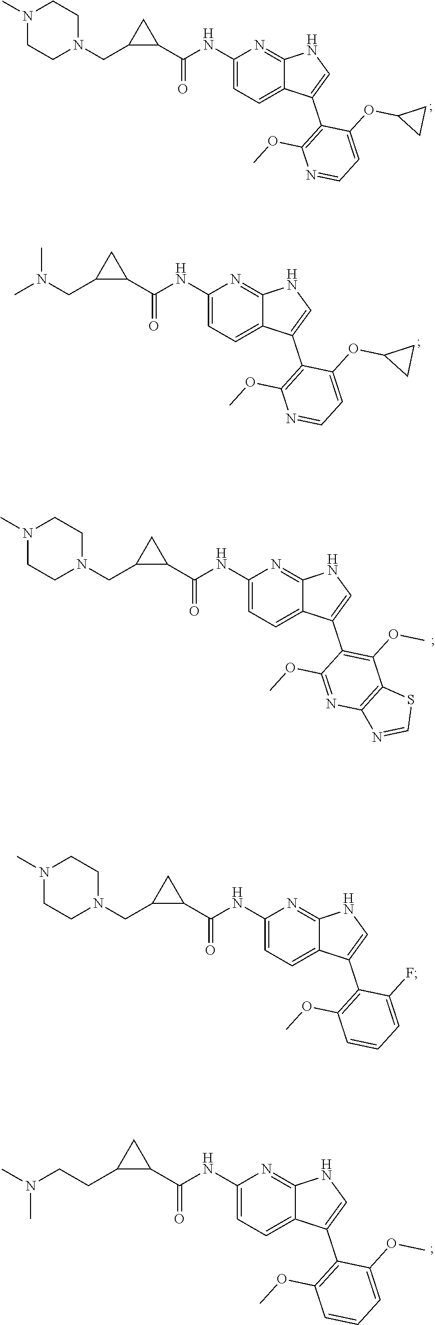 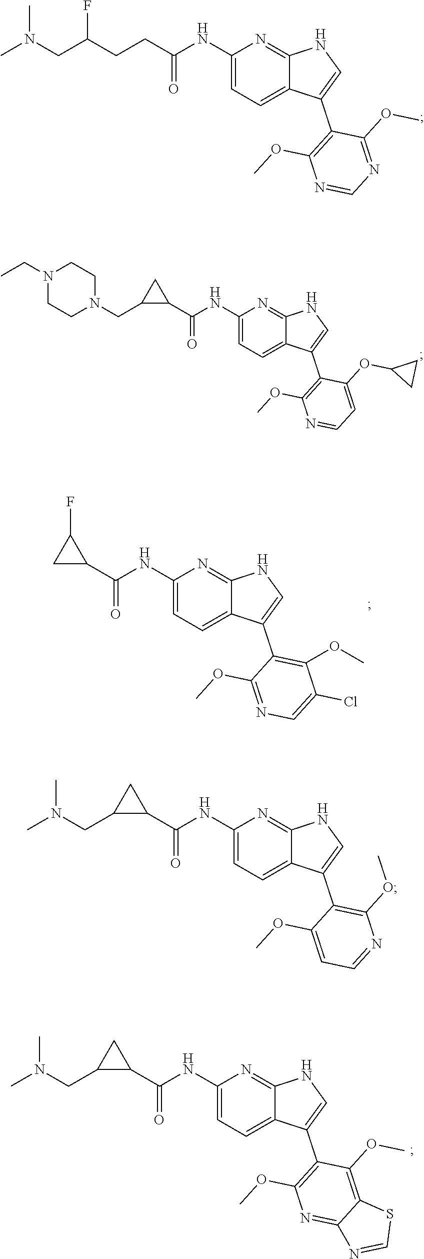 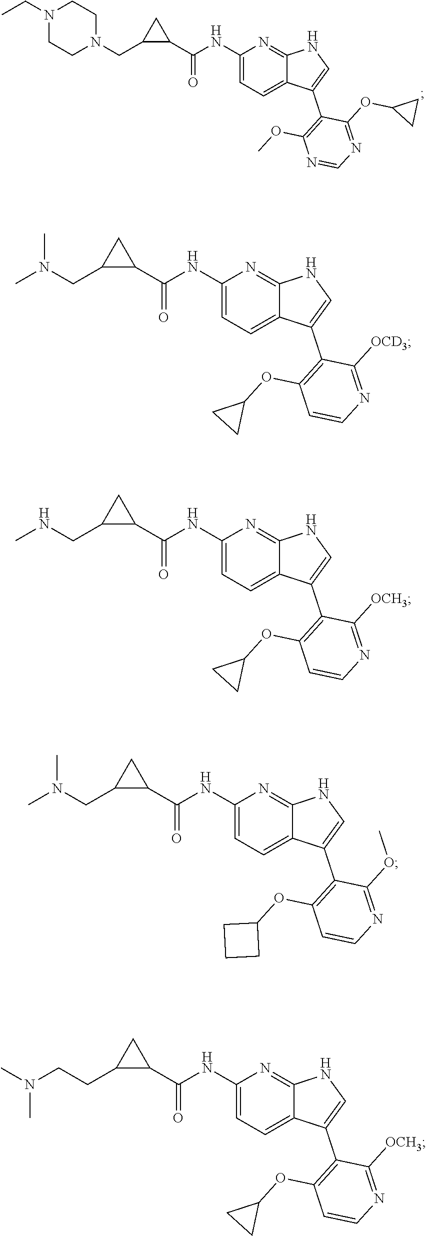 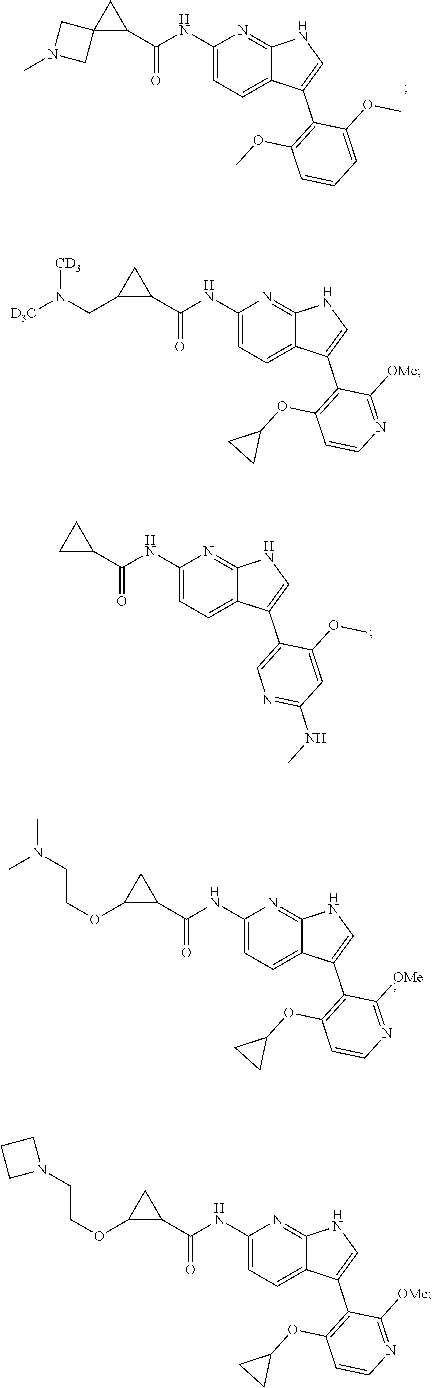 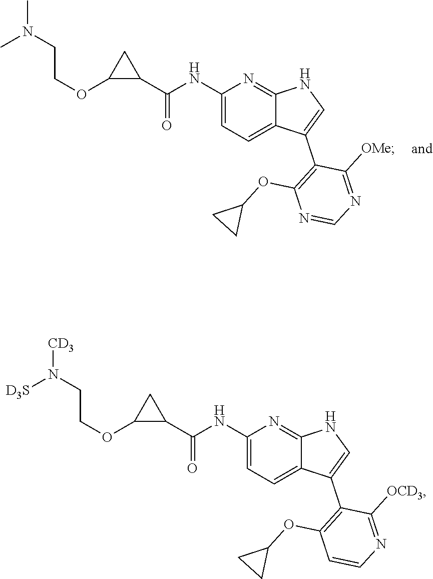
or a pharmaceutically acceptable salt, solvate, hydrate, or co-crystal thereof, or a mixture of any of the foregoing.

21. A compound selected from the group consisting of 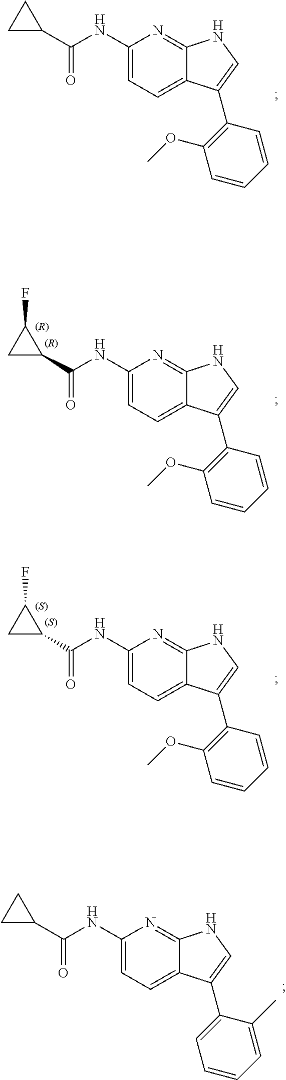 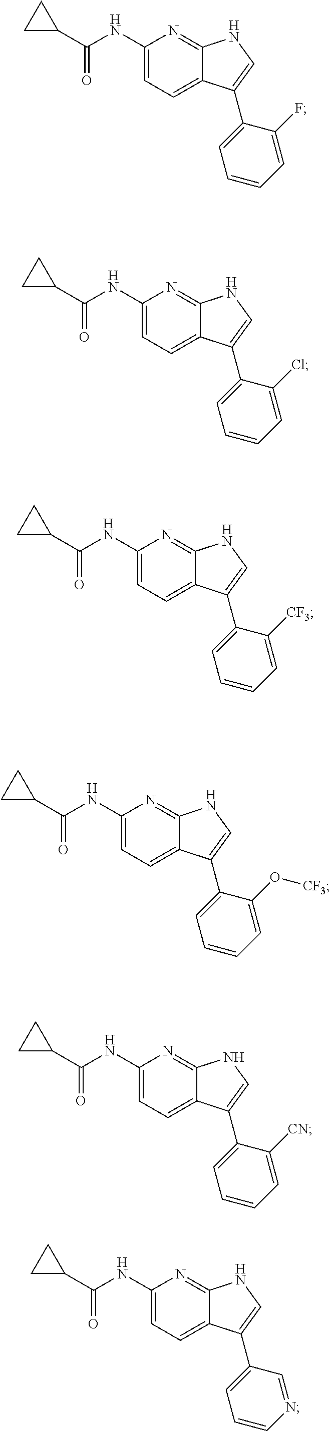 [SMILES: O=C(Nc1ccc2c(-c3ccccc3F)c[nH]c2n1)C1CC1 ; O=C(Nc1ccc2c(-c3ccccc3Cl)c[nH]c2n1)C1CC1 ; O=C(Nc1ccc2c(-c3ccccc3C(F)(F)F)c[nH]c2n1)C1CC1 ; O=C(Nc1ccc2c(-c3ccccc3OC(F)(F)F)c[nH]c2n1)C1CC1 ; N#Cc1ccccc1-c1c[nH]c2nc(NC(=O)C3CC3)ccc12 ; O=C(Nc1ccc2c(-c3cccnc3)c[nH]c2n1)C1CC1] 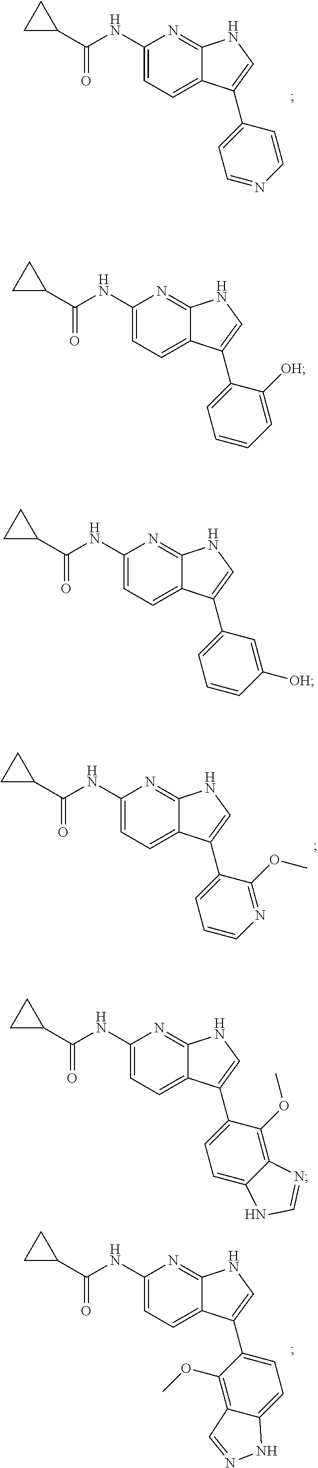 [SMILES: O=C(Nc1ccc2c(-c3ccncc3)c[nH]c2n1)C1CC1 ; O=C(Nc1ccc2c(-c3ccccc3O)c[nH]c2n1)C1CC1 ; O=C(Nc1ccc2c(-c3cccc(O)c3)c[nH]c2n1)C1CC1 ; COc1ncccc1-c1c[nH]c2nc(NC(=O)C3CC3)ccc12 ; COc1c(-c2c[nH]c3nc(NC(=O)C4CC4)ccc23)ccc2[nH]cnc12 ; COc1c(-c2c[nH]c3nc(NC(=O)C4CC4)ccc23)ccc2[nH]ncc12] 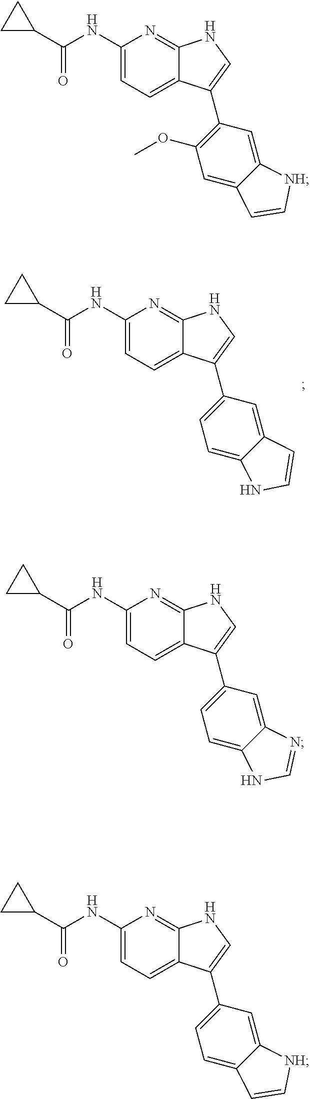 [SMILES: COc1cc2cc[nH]c2cc1-c1c[nH]c2nc(NC(=O)C3CC3)ccc12 ; O=C(Nc1ccc2c(-c3ccc4[nH]ccc4c3)c[nH]c2n1)C1CC1 ; O=C(Nc1ccc2c(-c3ccc4[nH]cnc4c3)c[nH]c2n1)C1CC1 ; O=C(Nc1ccc2c(-c3ccc4cc[nH]c4c3)c[nH]c2n1)C1CC1] 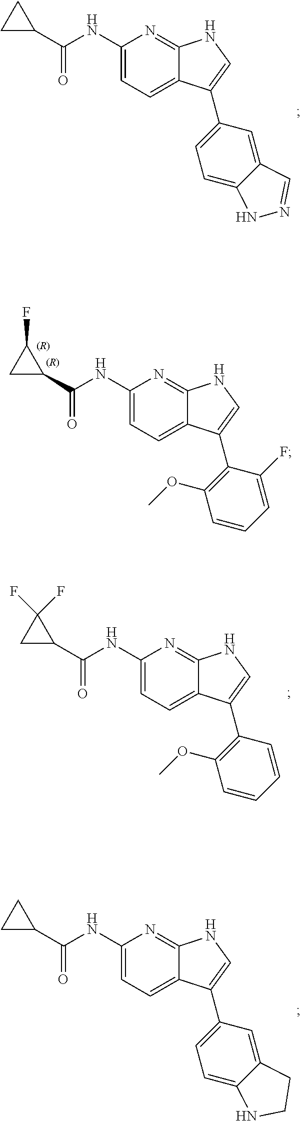 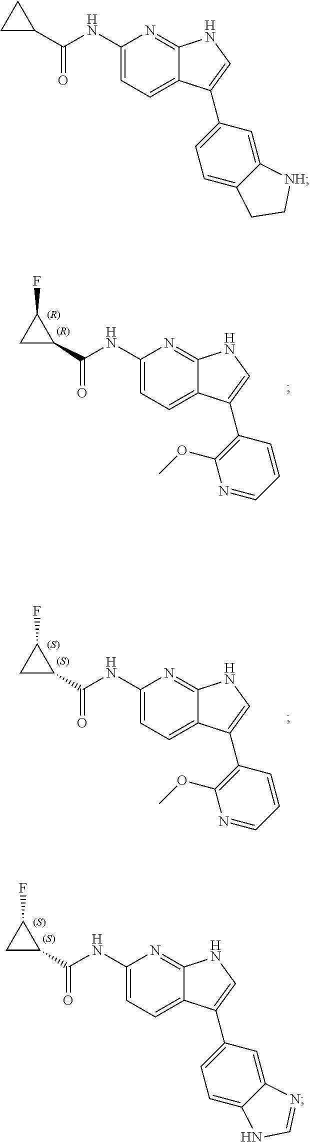 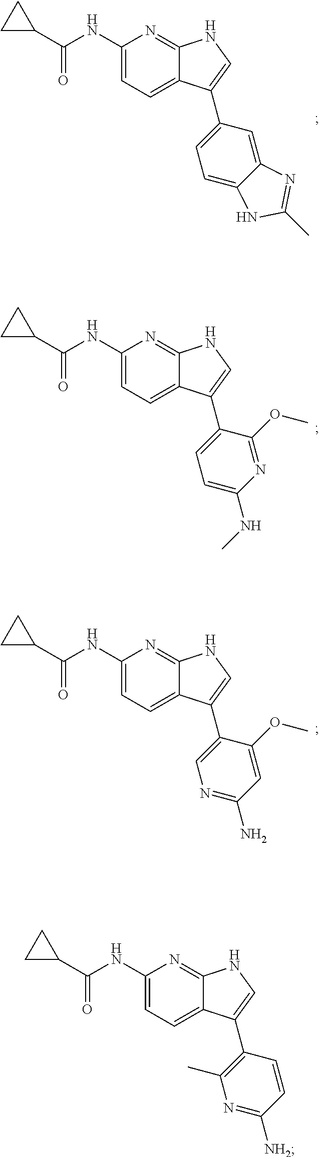 [SMILES: Cc1nc2cc(-c3c[nH]c4nc(NC(=O)C5CC5)ccc34)ccc2[nH]1 ; CNc1ccc(-c2c[nH]c3nc(NC(=O)C4CC4)ccc23)c(OC)n1 ; COc1cc(N)ncc1-c1c[nH]c2nc(NC(=O)C3CC3)ccc12 ; Cc1nc(N)ccc1-c1c[nH]c2nc(NC(=O)C3CC3)ccc12] 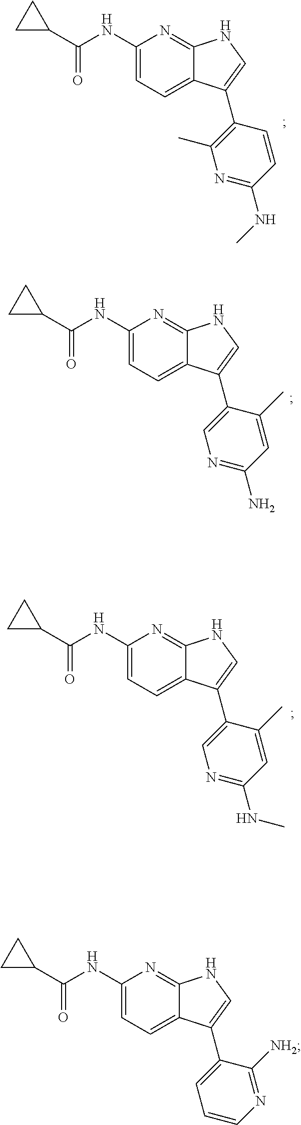 [SMILES: CNc1ccc(-c2c[nH]c3nc(NC(=O)C4CC4)ccc23)c(C)n1 ; Cc1cc(N)ncc1-c1c[nH]c2nc(NC(=O)C3CC3)ccc12 ; CNc1cc(C)c(-c2c[nH]c3nc(NC(=O)C4CC4)ccc23)cn1 ; Nc1ncccc1-c1c[nH]c2nc(NC(=O)C3CC3)ccc12] 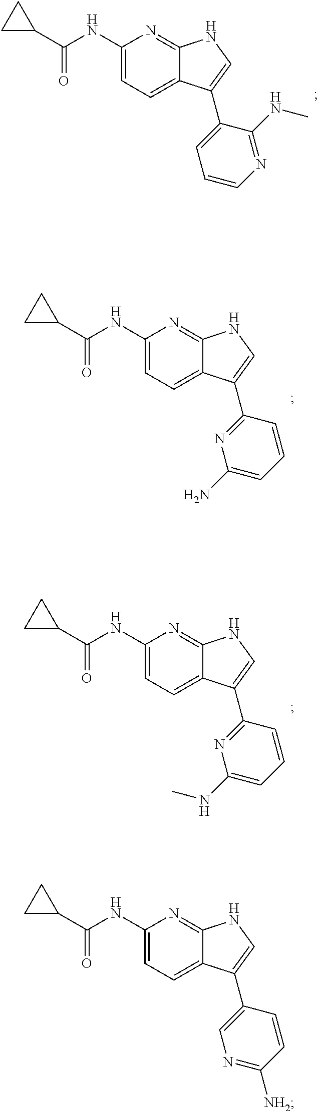 [SMILES: CNc1ncccc1-c1c[nH]c2nc(NC(=O)C3CC3)ccc12 ; Nc1cccc(-c2c[nH]c3nc(NC(=O)C4CC4)ccc23)n1 ; CNc1cccc(-c2c[nH]c3nc(NC(=O)C4CC4)ccc23)n1 ; Nc1ccc(-c2c[nH]c3nc(NC(=O)C4CC4)ccc23)cn1] 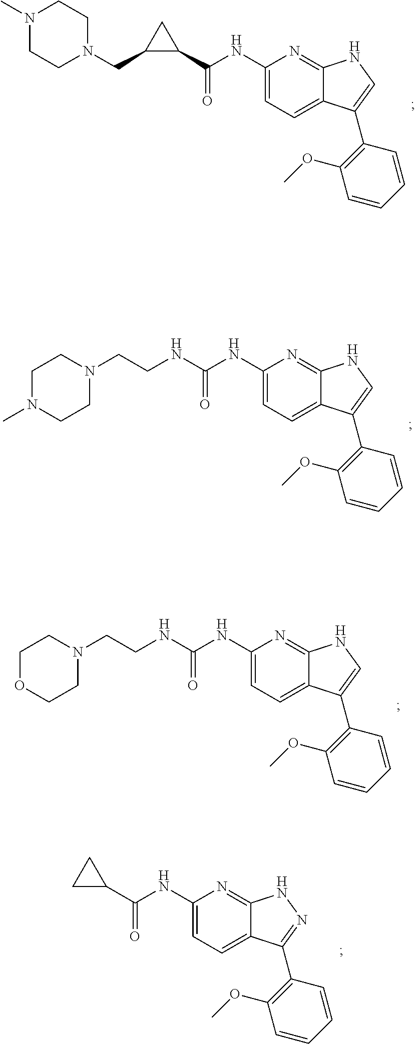 [SMILES: COc1ccccc1-c1c[nH]c2nc(NC(=O)[C@@H]3C[C@@H]3CN3CCN(C)CC3)ccc12 ; COc1ccccc1-c1c[nH]c2nc(NC(=O)NCCN3CCN(C)CC3)ccc12 ; COc1ccccc1-c1c[nH]c2nc(NC(=O)NCCN3CCOCC3)ccc12 ; COc1ccccc1-c1n[nH]c2nc(NC(=O)C3CC3)ccc12] 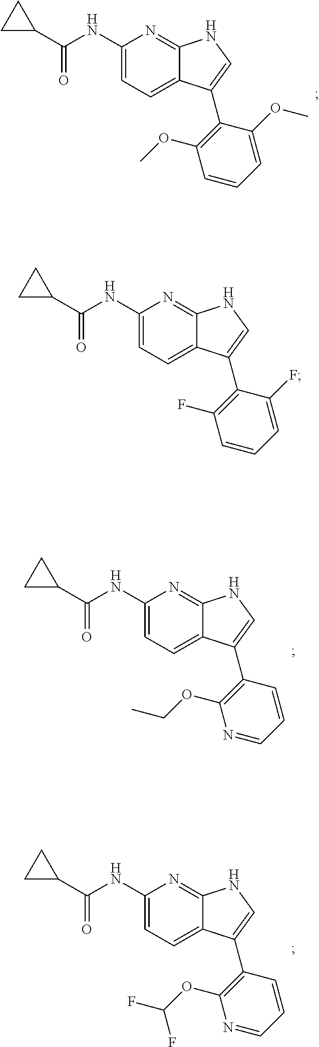 [SMILES: COc1cccc(OC)c1-c1c[nH]c2nc(NC(=O)C3CC3)ccc12 ; O=C(Nc1ccc2c(-c3c(F)cccc3F)c[nH]c2n1)C1CC1 ; CCOc1ncccc1-c1c[nH]c2nc(NC(=O)C3CC3)ccc12 ; O=C(Nc1ccc2c(-c3cccnc3OC(F)F)c[nH]c2n1)C1CC1] 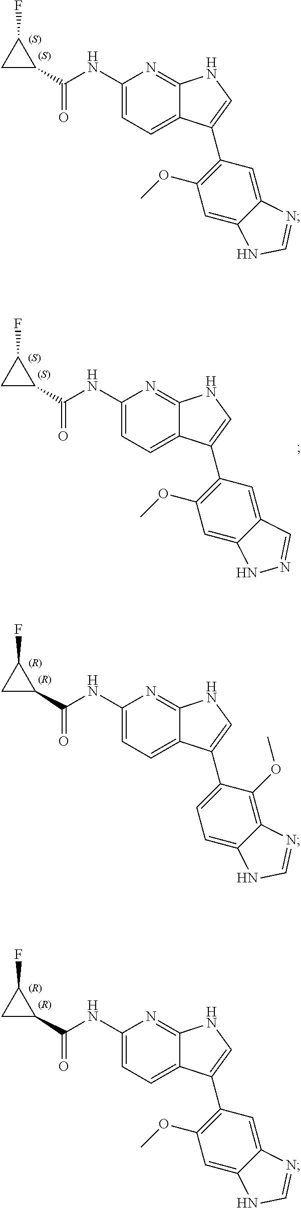 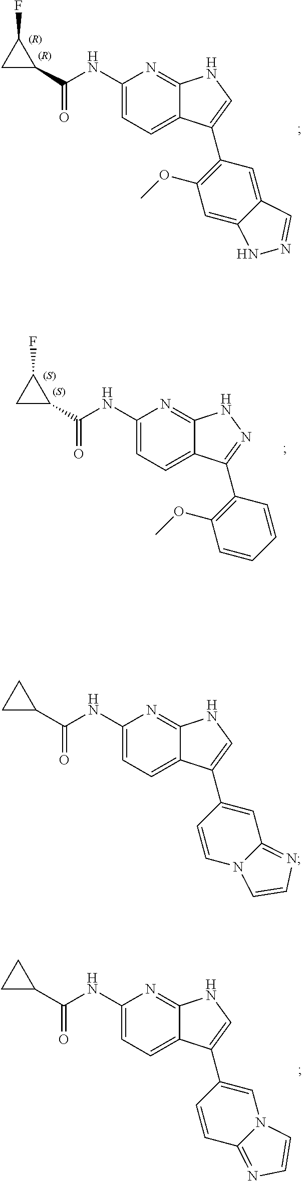 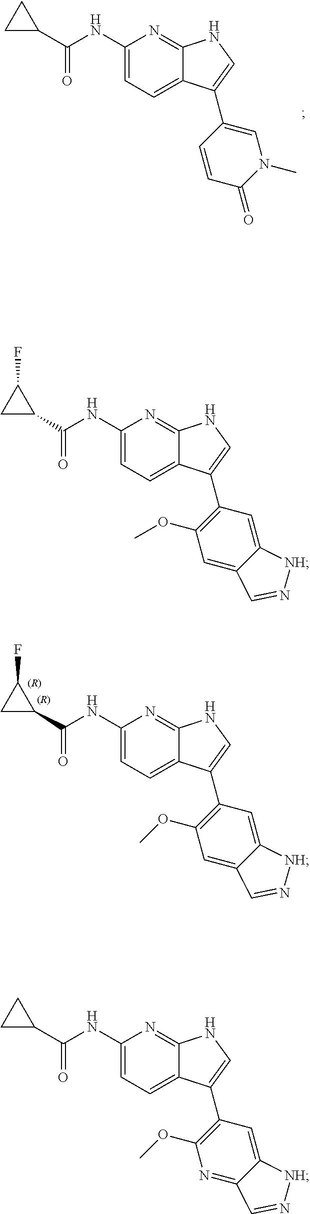 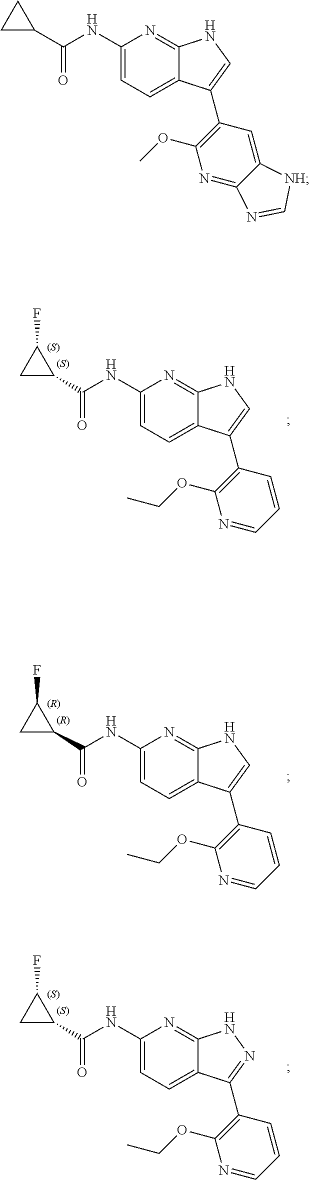 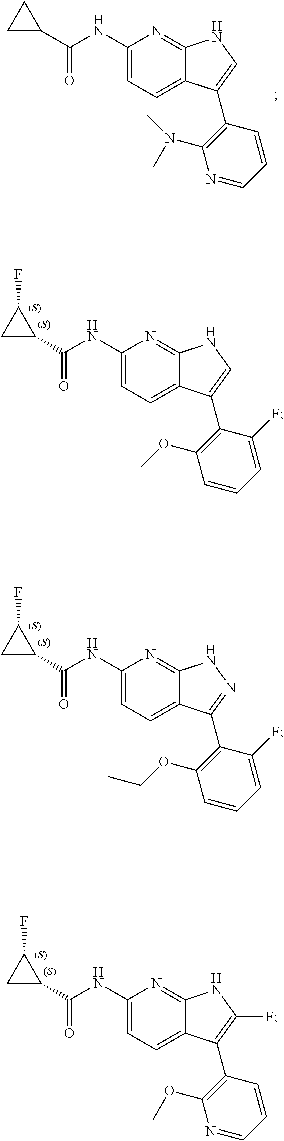 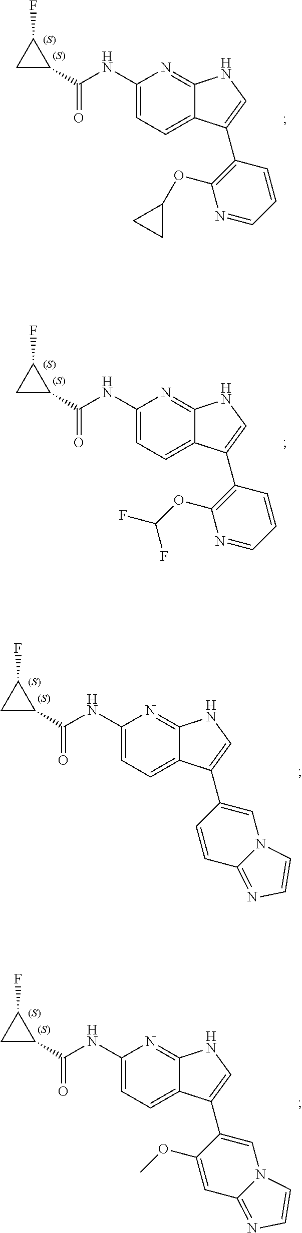 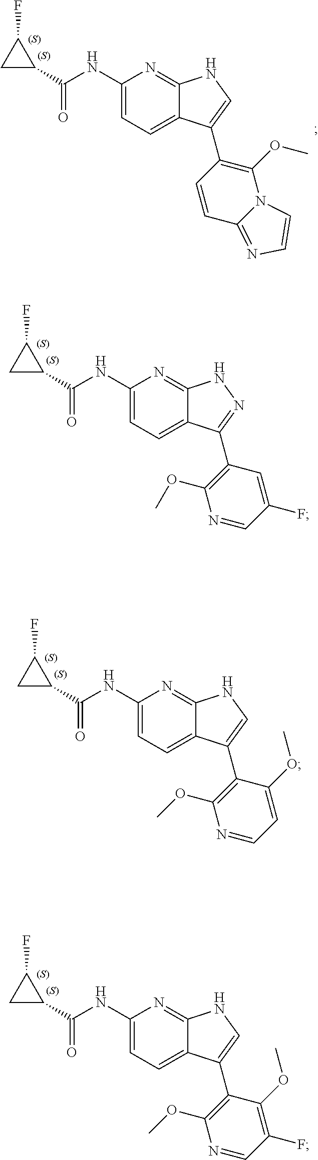 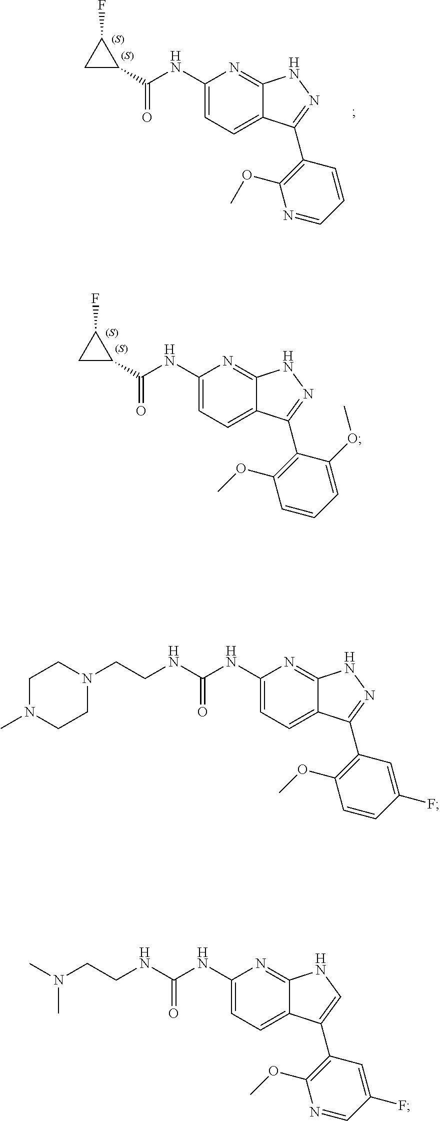 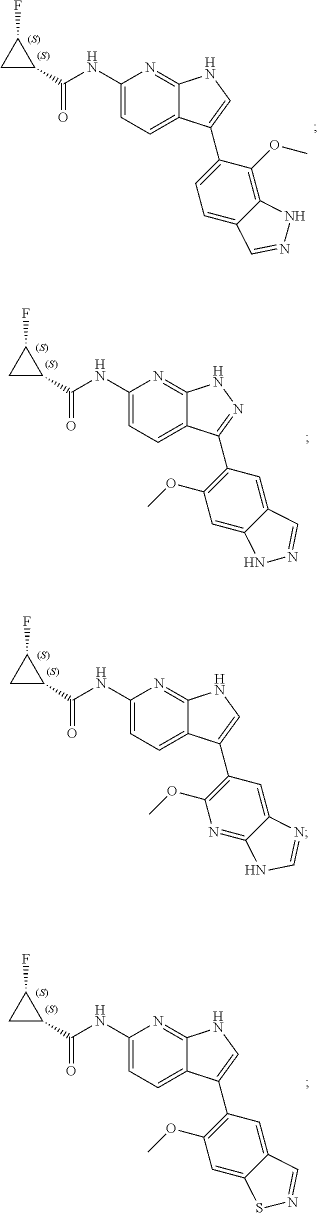 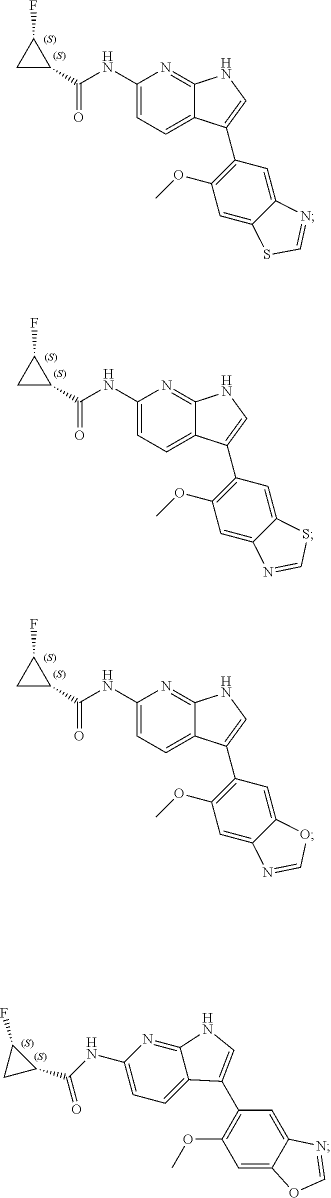 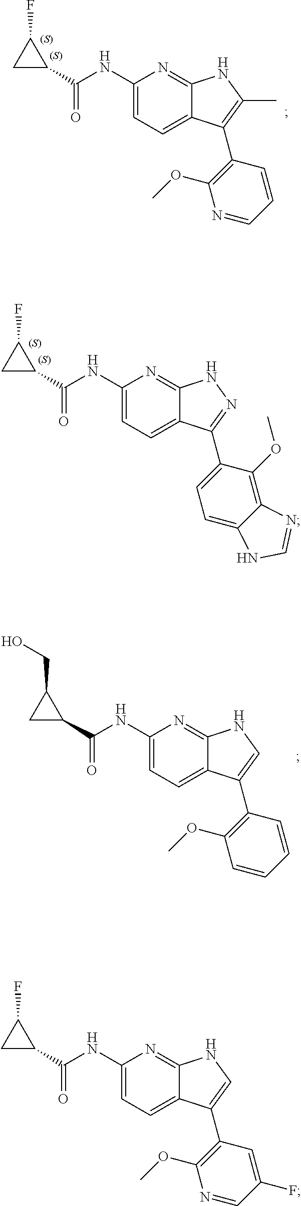 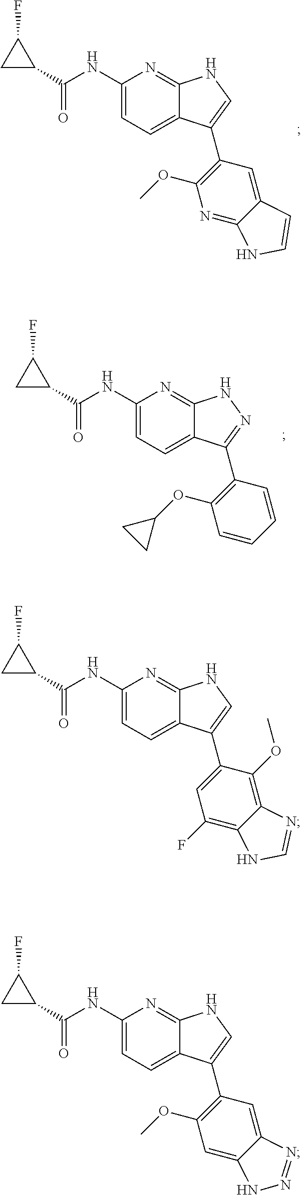 [SMILES: COc1nc2[nH]ccc2cc1-c1c[nH]c2nc(NC(=O)[C@@H]3C[C@@H]3F)ccc12 ; O=C(Nc1ccc2c(-c3ccccc3OC3CC3)n[nH]c2n1)[C@@H]1C[C@@H]1F ; COc1c(-c2c[nH]c3nc(NC(=O)[C@@H]4C[C@@H]4F)ccc23)cc(F)c2[nH]cnc12 ; COc1cc2[nH]nnc2cc1-c1c[nH]c2nc(NC(=O)[C@@H]3C[C@@H]3F)ccc12] 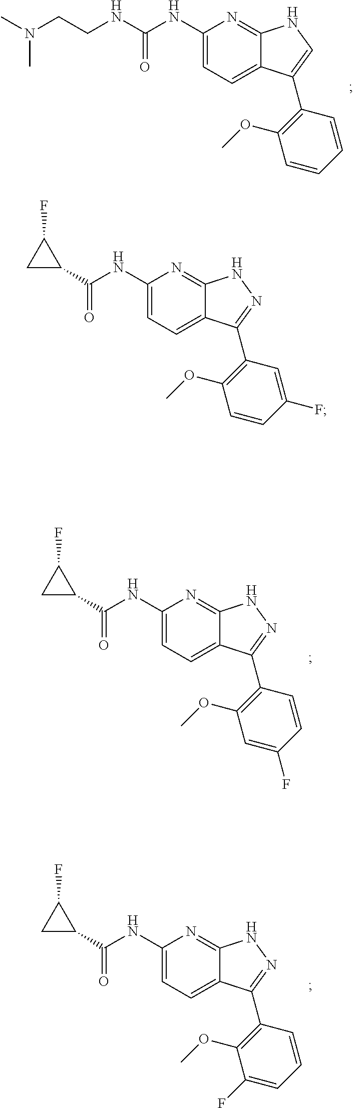 [SMILES: COc1ccccc1-c1c[nH]c2nc(NC(=O)NCCN(C)C)ccc12 ; COc1ccc(F)cc1-c1n[nH]c2nc(NC(=O)[C@@H]3C[C@@H]3F)ccc12 ; COc1cc(F)ccc1-c1n[nH]c2nc(NC(=O)[C@@H]3C[C@@H]3F)ccc12 ; COc1c(F)cccc1-c1n[nH]c2nc(NC(=O)[C@@H]3C[C@@H]3F)ccc12] 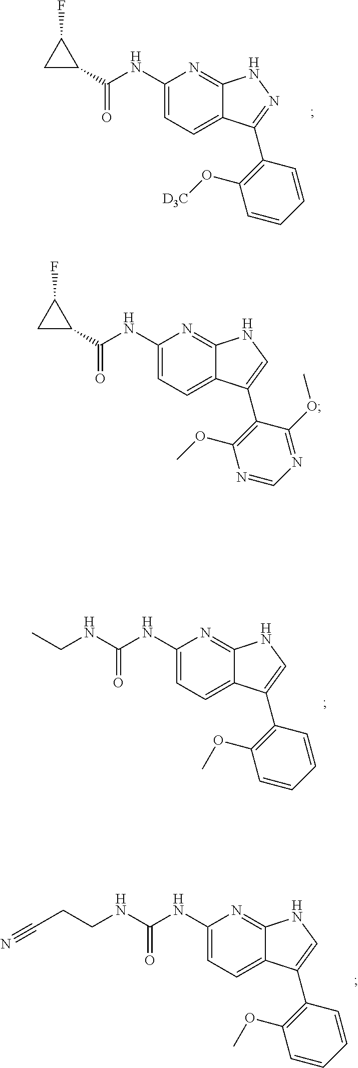 [SMILES: [2H]C([2H])([2H])Oc1ccccc1-c1n[nH]c2nc(NC(=O)[C@@H]3C[C@@H]3F)ccc12 ; COc1ncnc(OC)c1-c1c[nH]c2nc(NC(=O)[C@@H]3C[C@@H]3F)ccc12 ; CCNC(=O)Nc1ccc2c(-c3ccccc3OC)c[nH]c2n1 ; COc1ccccc1-c1c[nH]c2nc(NC(=O)NCCC#N)ccc12] 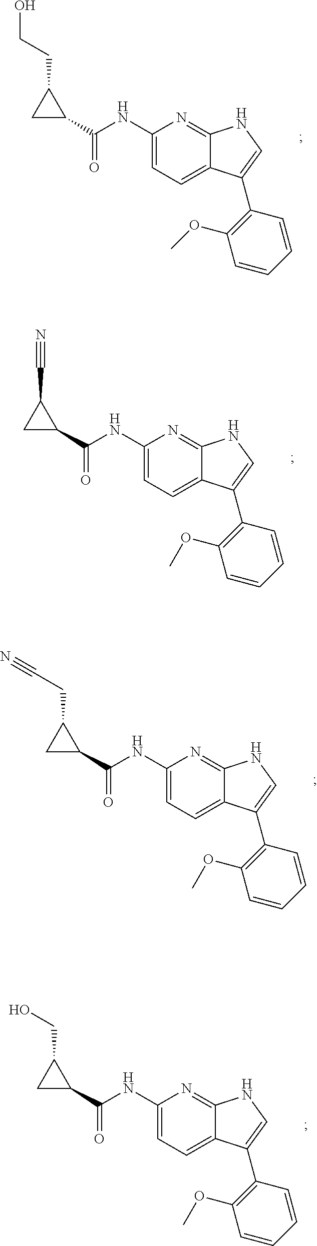 [SMILES: COc1ccccc1-c1c[nH]c2nc(NC(=O)[C@@H]3C[C@@H]3CCO)ccc12 ; COc1ccccc1-c1c[nH]c2nc(NC(=O)[C@H]3C[C@H]3C#N)ccc12 ; COc1ccccc1-c1c[nH]c2nc(NC(=O)[C@H]3C[C@@H]3CC#N)ccc12 ; COc1ccccc1-c1c[nH]c2nc(NC(=O)[C@H]3C[C@@H]3CO)ccc12] 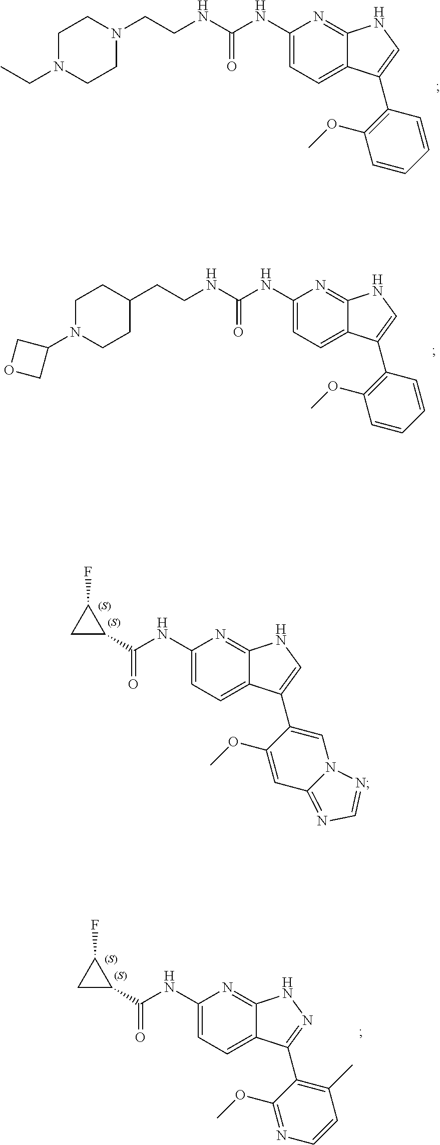 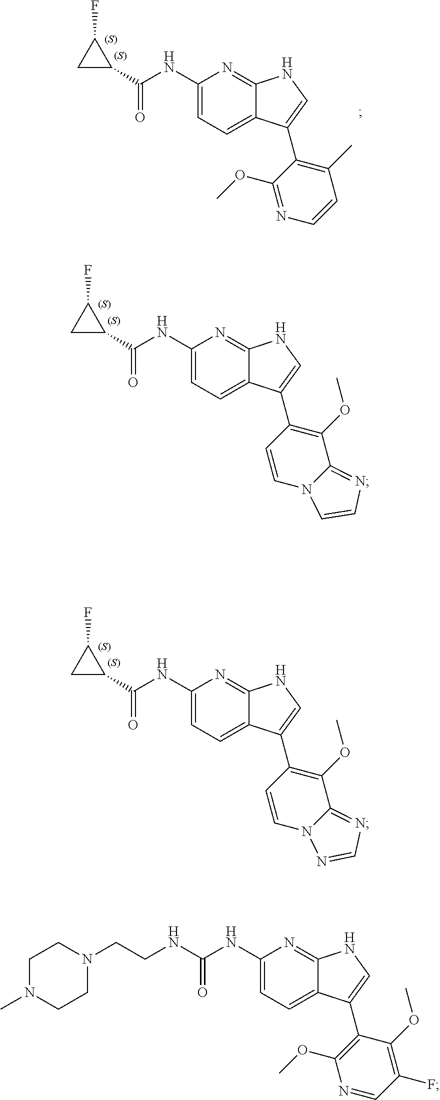 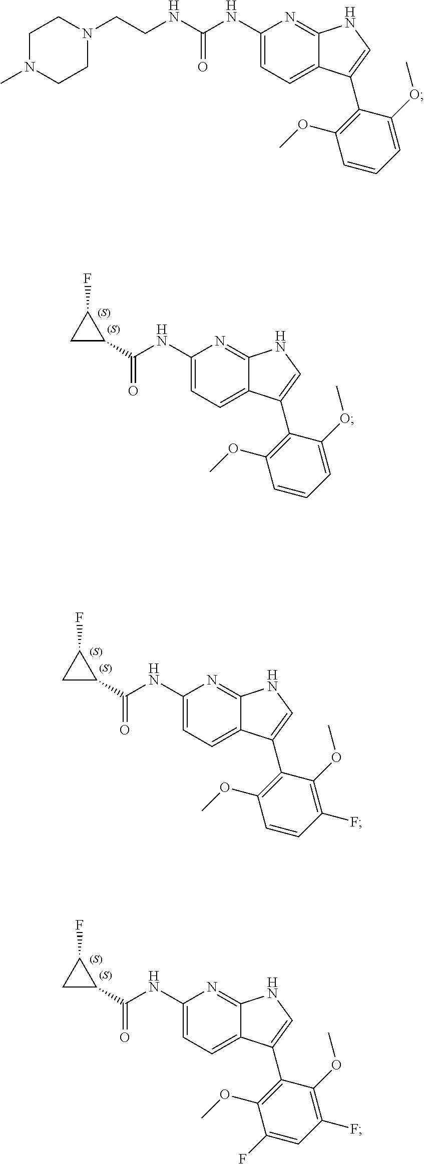 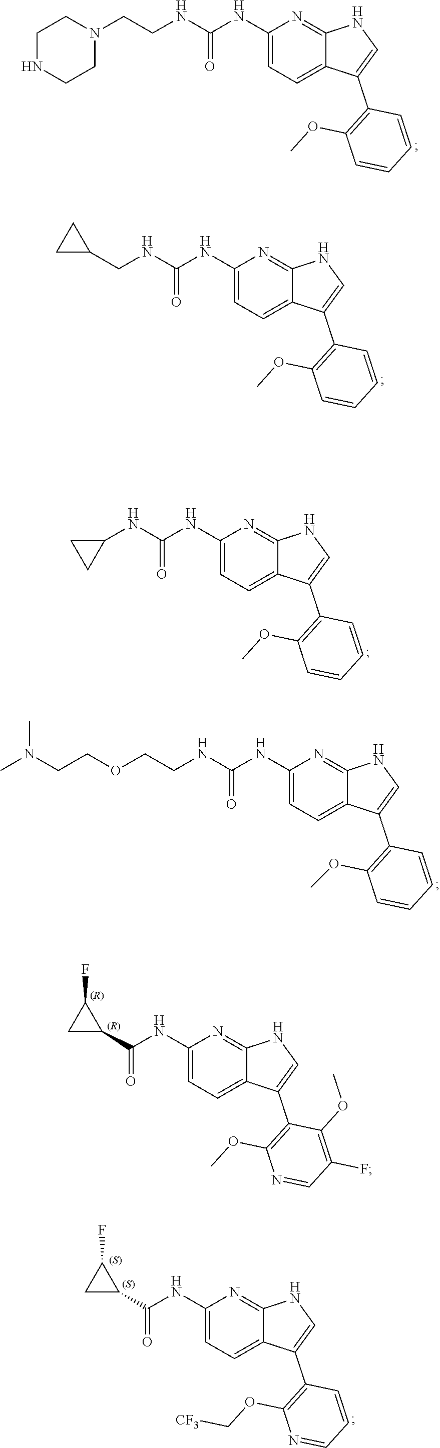 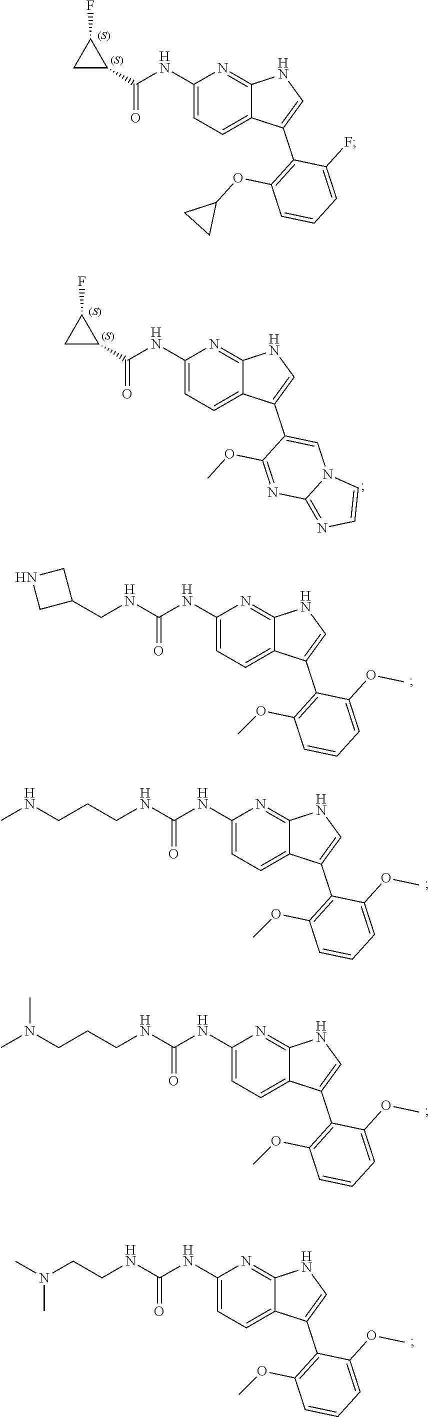 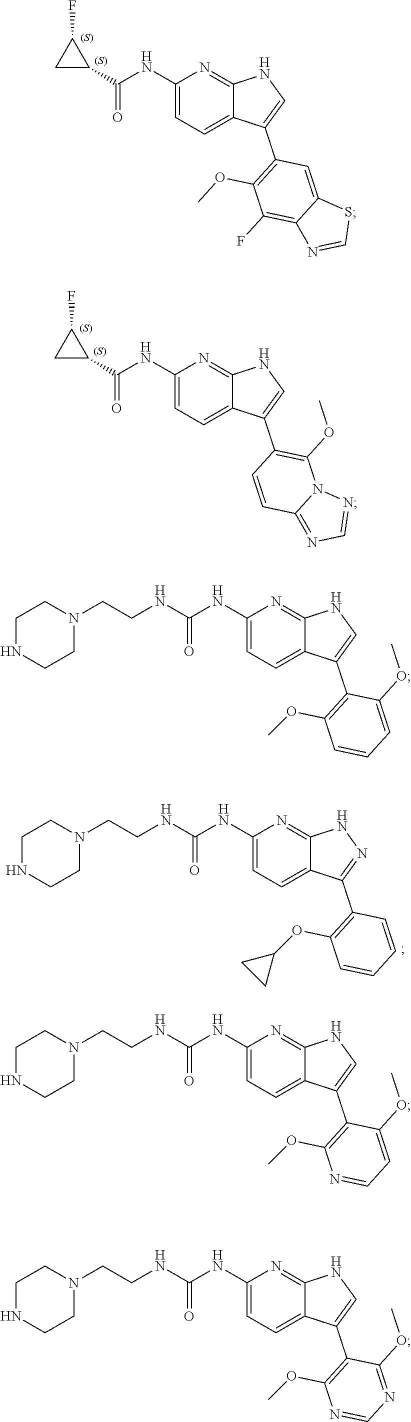 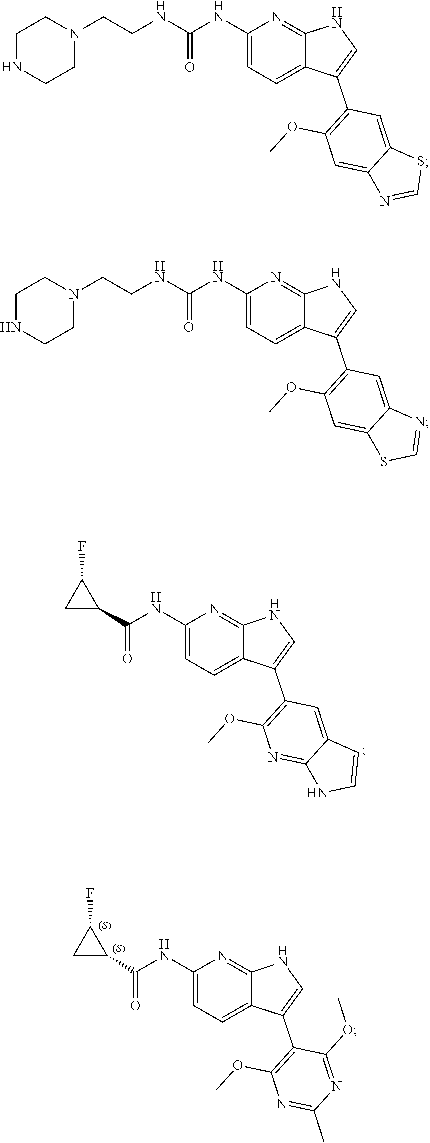 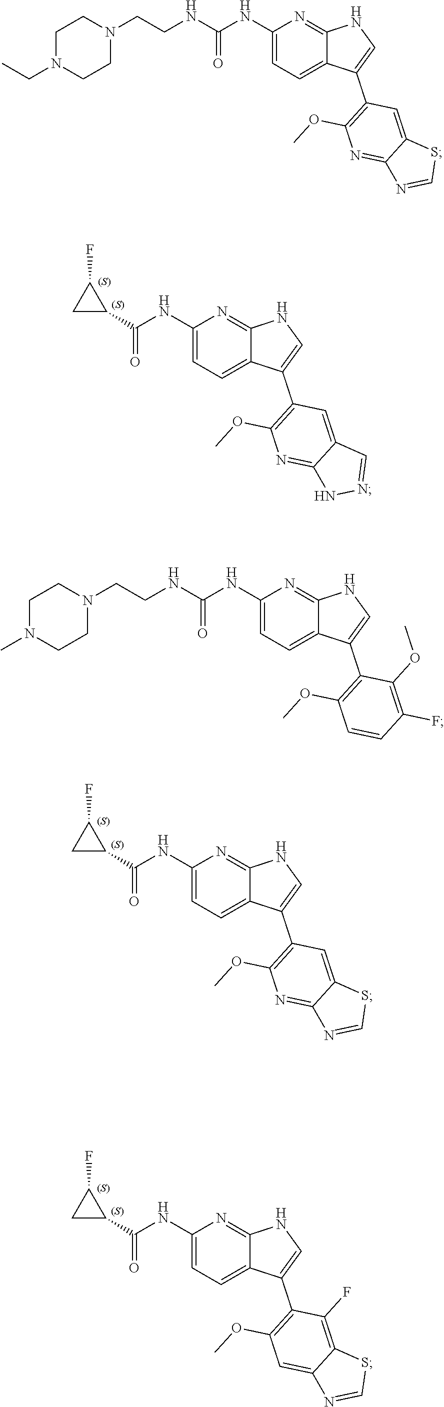 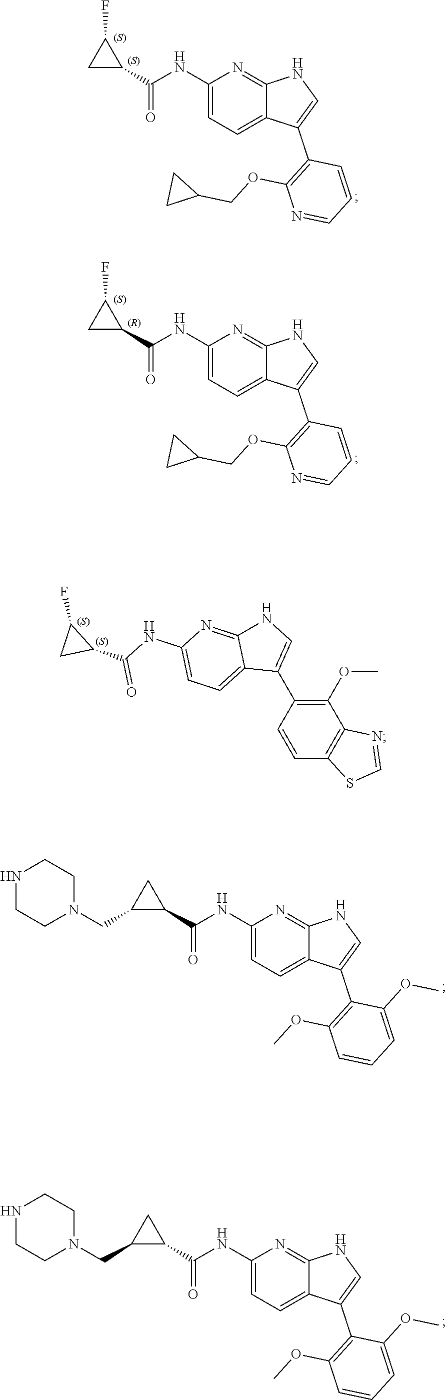 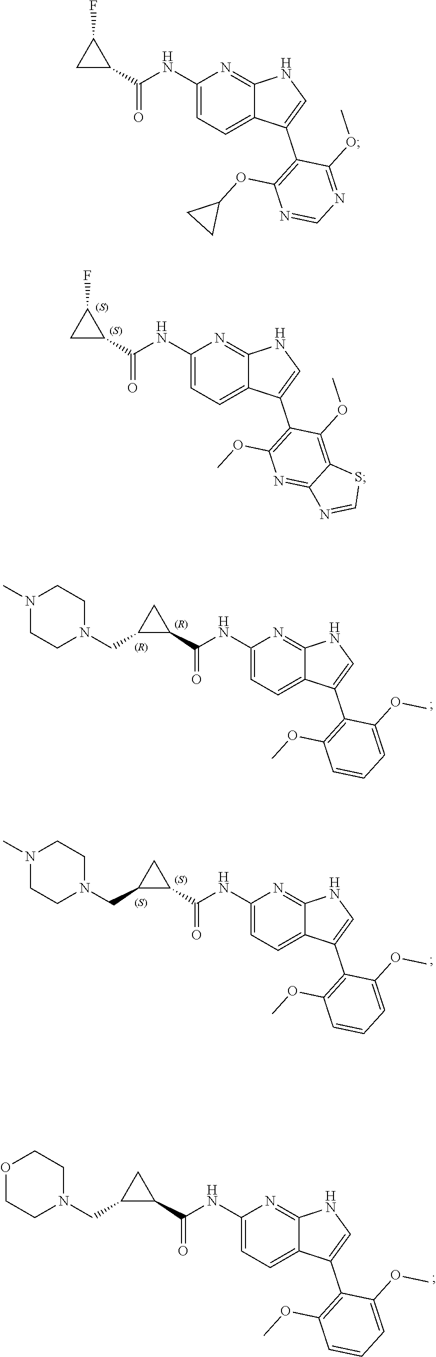 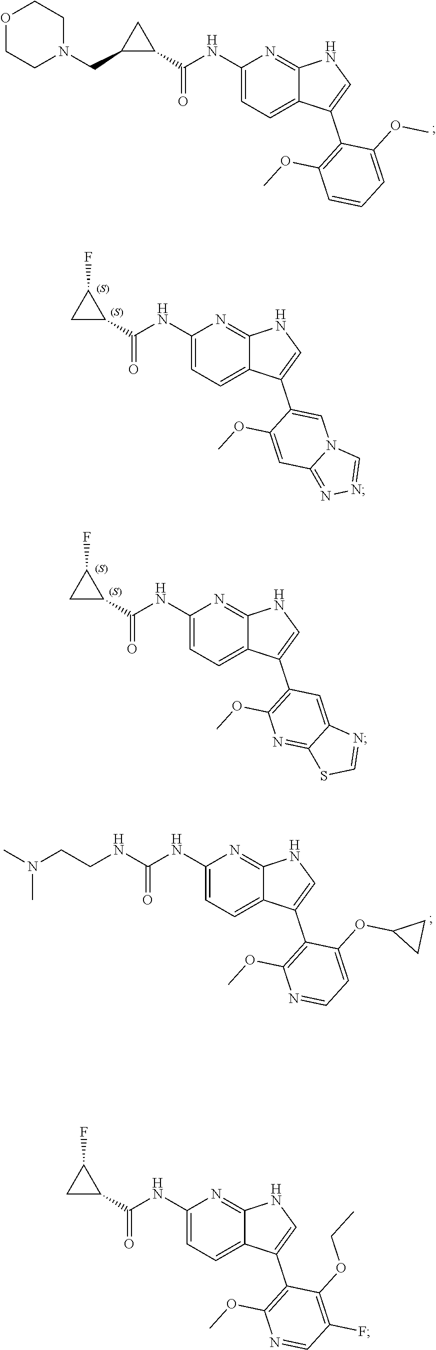 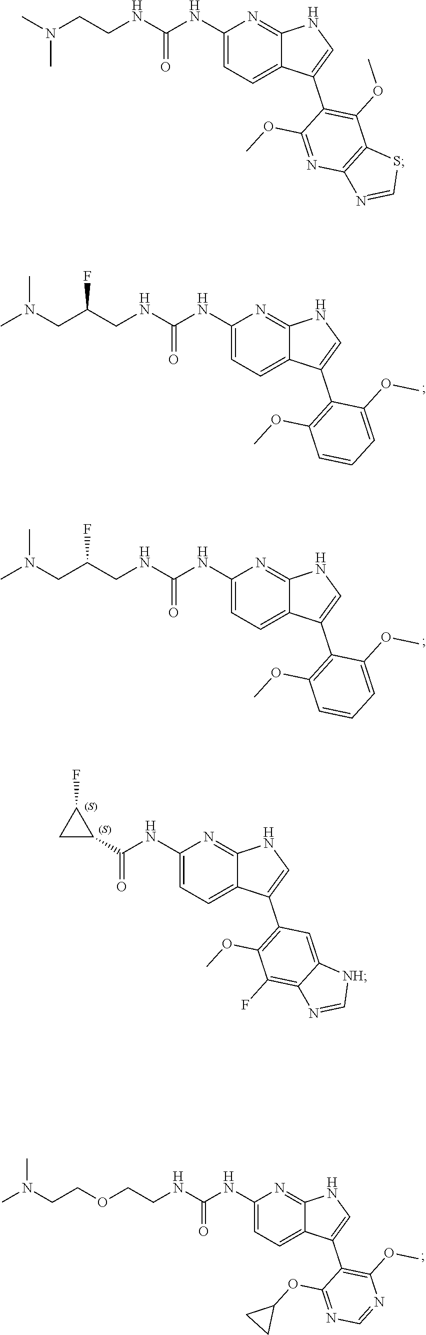 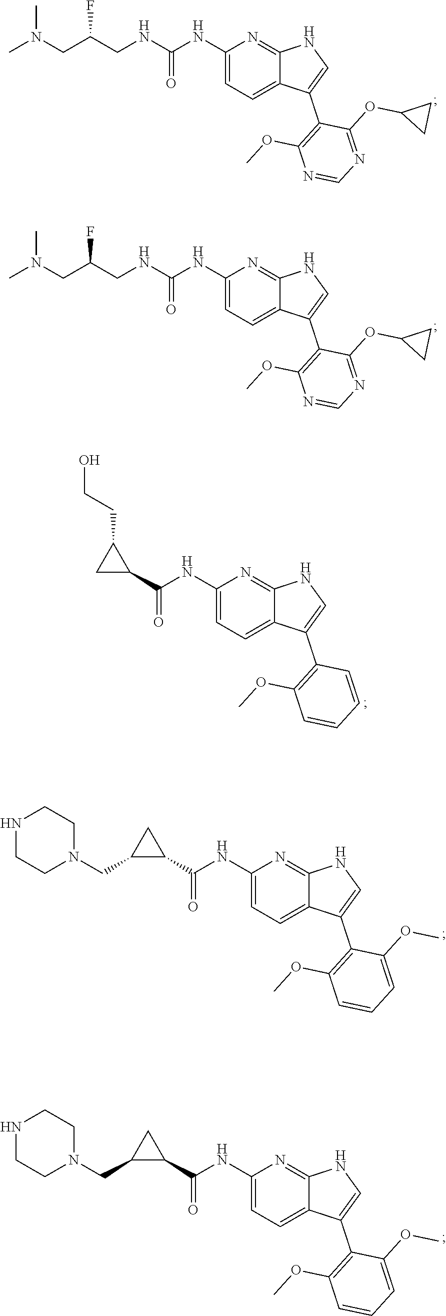 [SMILES: COc1ncnc(OC2CC2)c1-c1c[nH]c2nc(NC(=O)NC[C@@H](F)CN(C)C)ccc12 ; COc1ncnc(OC2CC2)c1-c1c[nH]c2nc(NC(=O)NC[C@H](F)CN(C)C)ccc12 ; COc1ccccc1-c1c[nH]c2nc(NC(=O)[C@H]3C[C@@H]3CCO)ccc12 ; COc1cccc(OC)c1-c1c[nH]c2nc(NC(=O)[C@H]3C[C@H]3CN3CCNCC3)ccc12 ; COc1cccc(OC)c1-c1c[nH]c2nc(NC(=O)[C@@H]3C[C@@H]3CN3CCNCC3)ccc12] 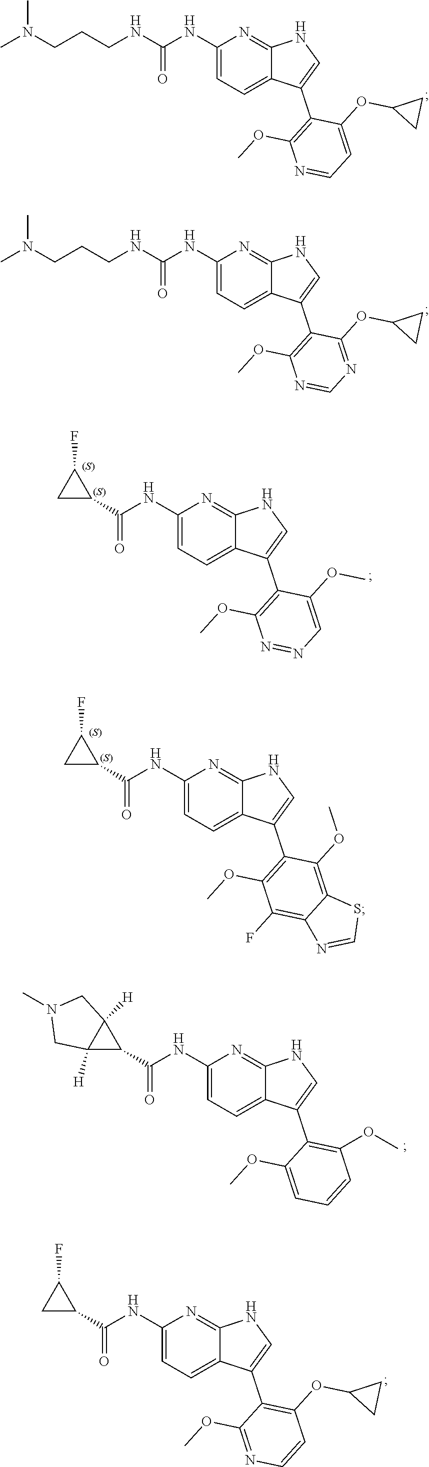 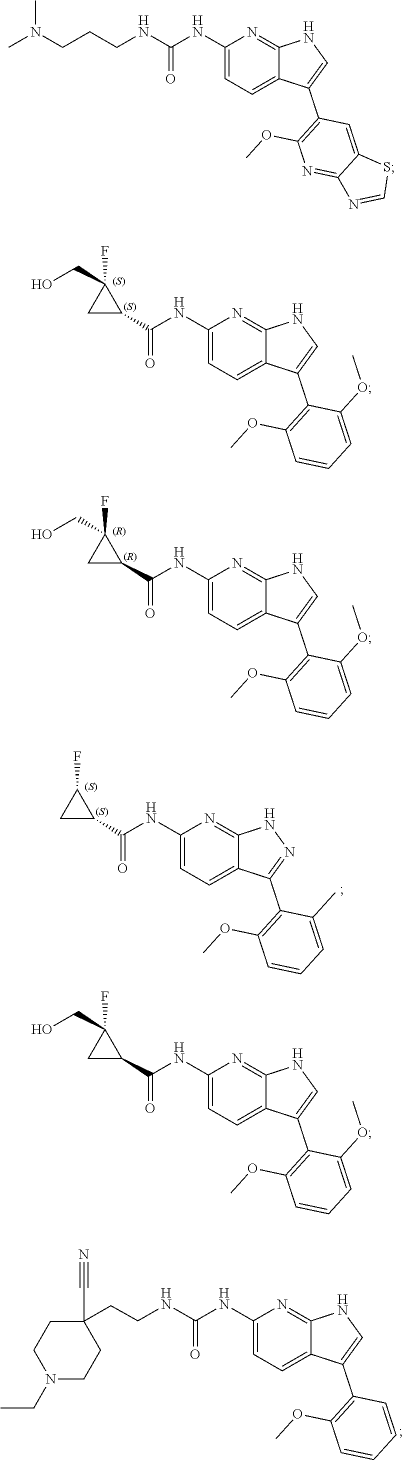 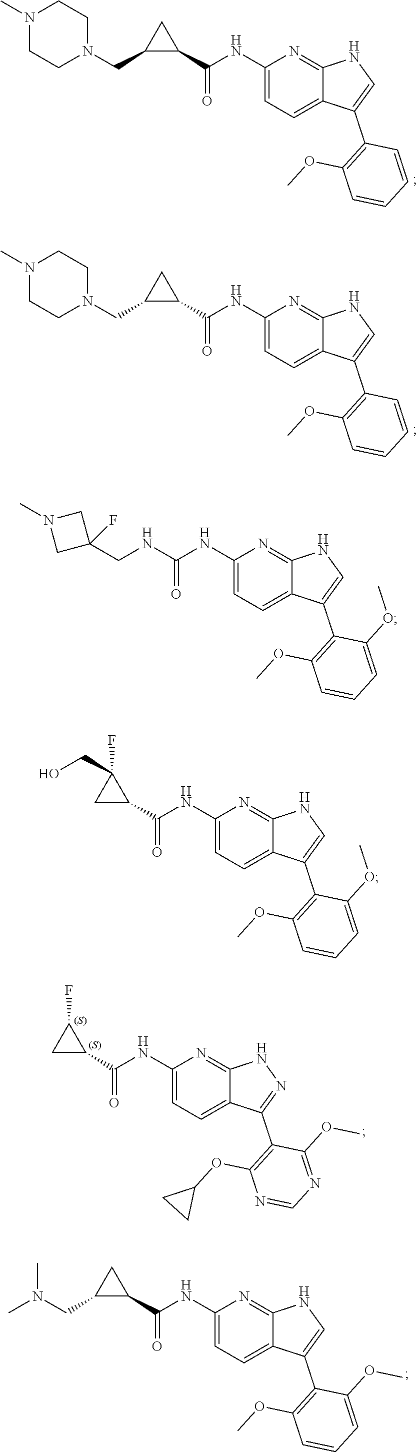 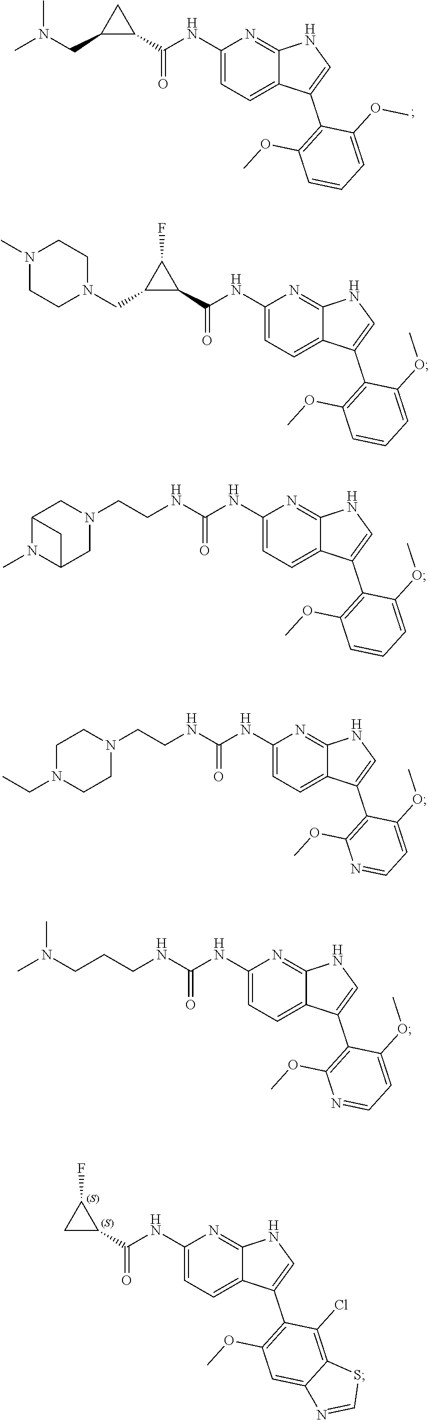 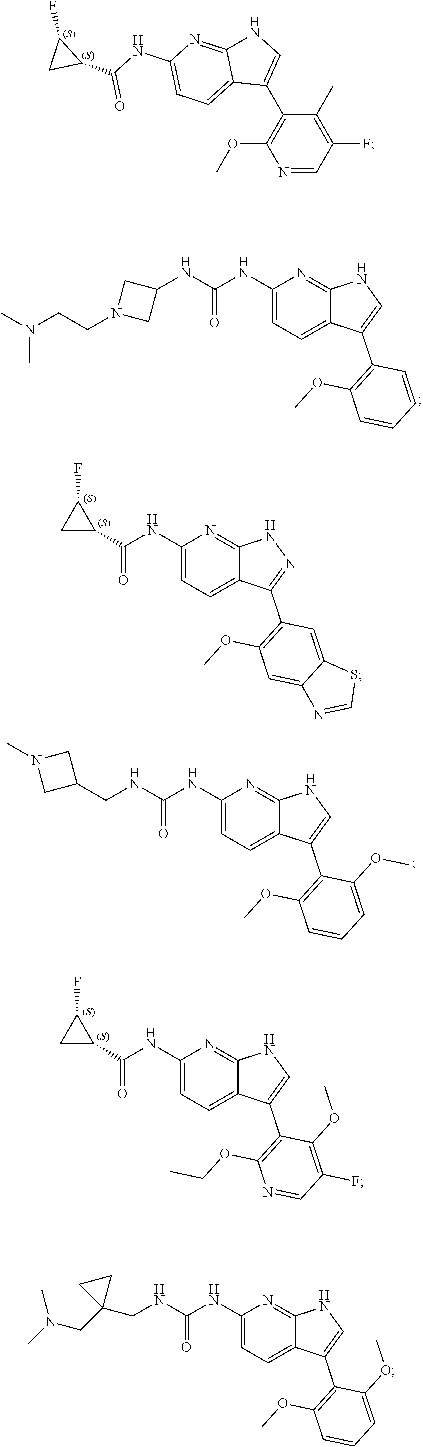 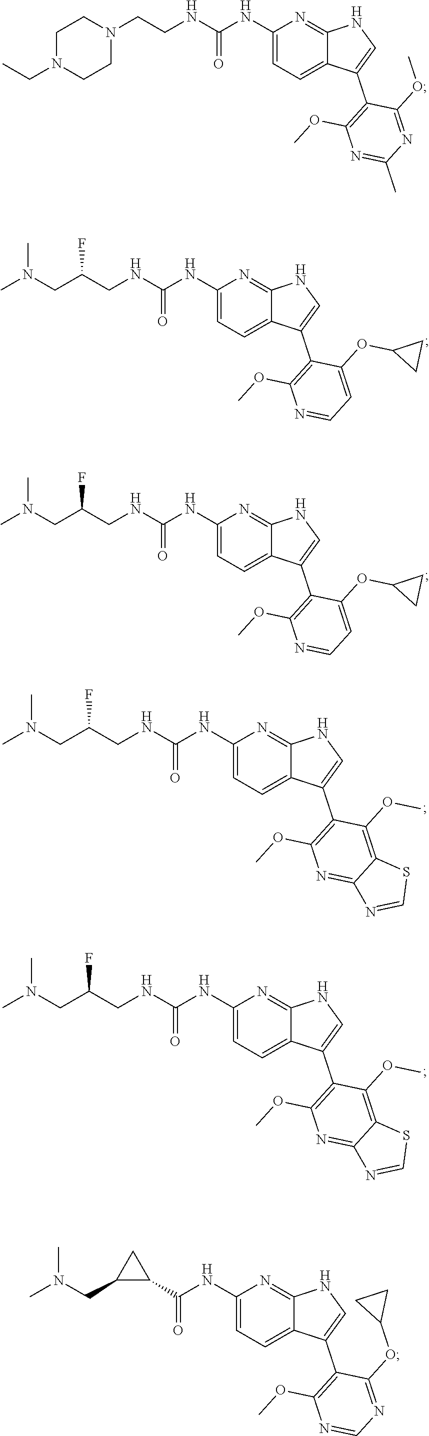 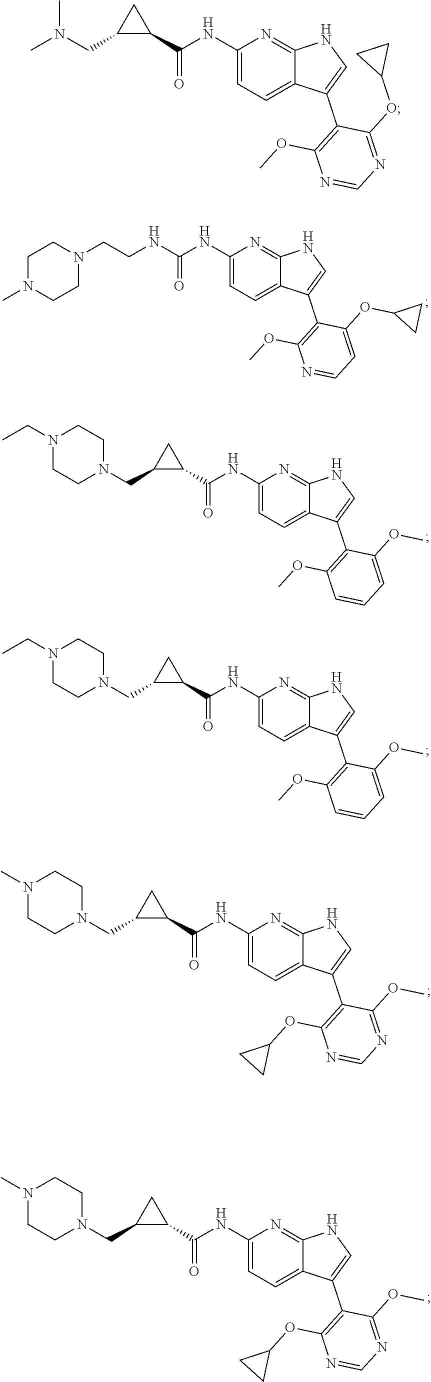 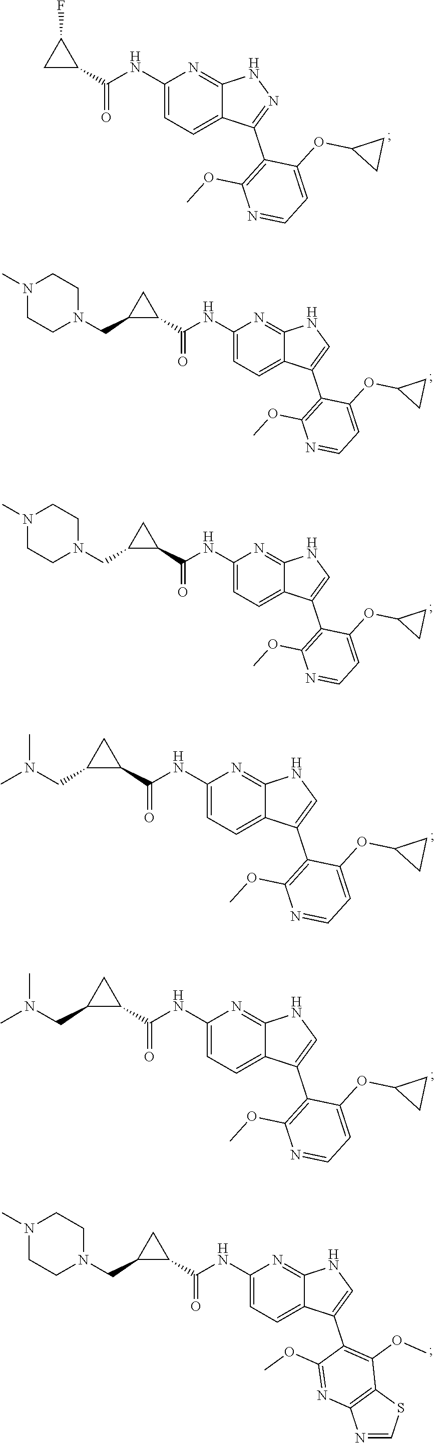 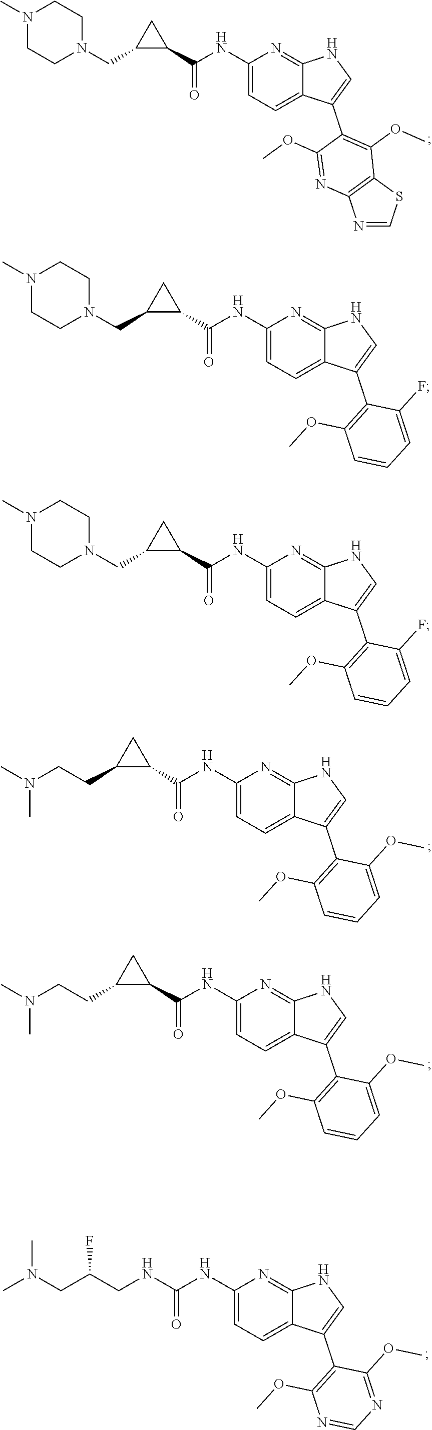 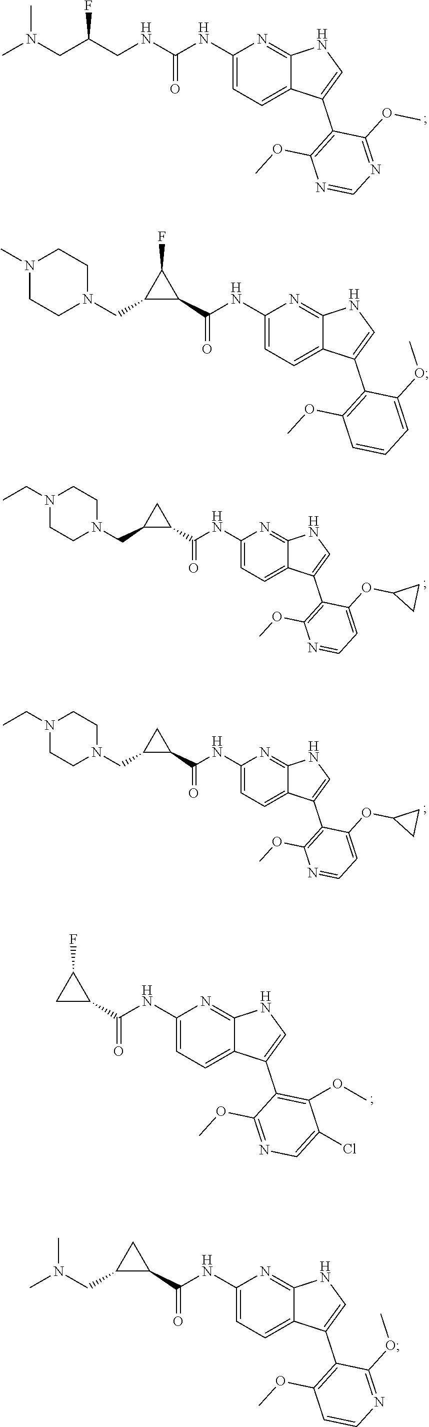 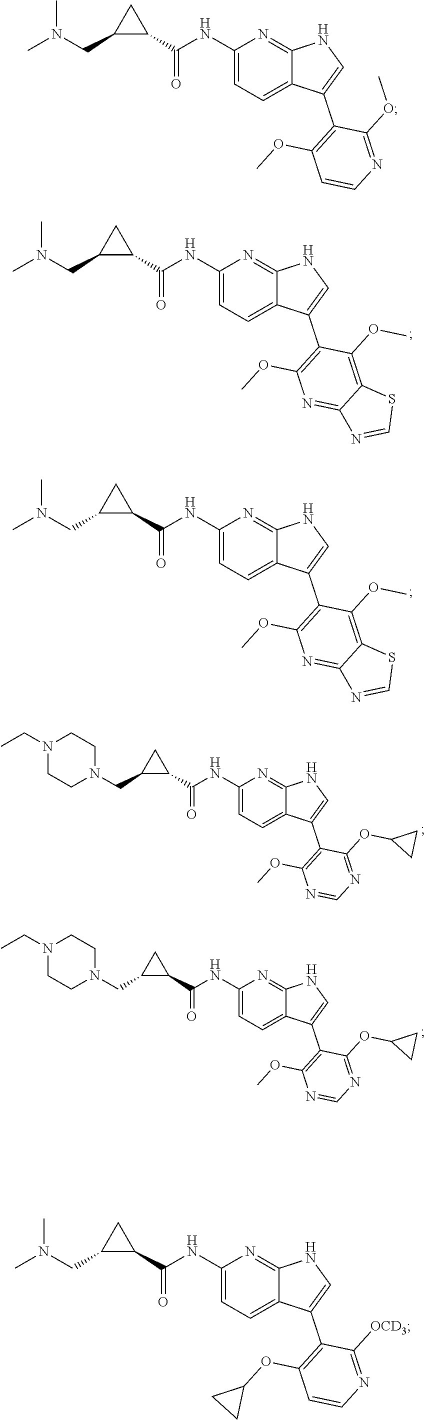 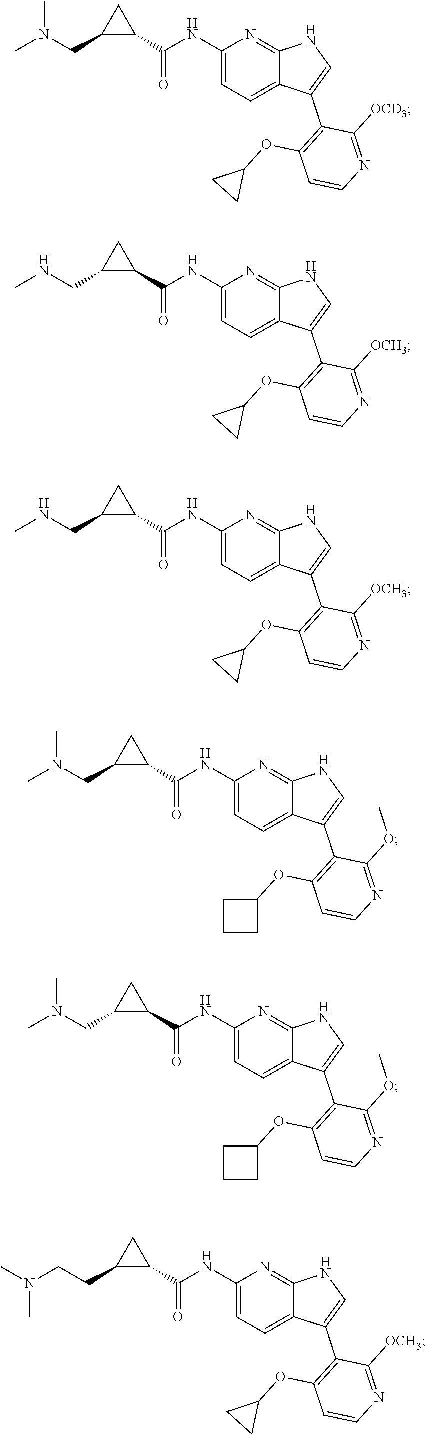 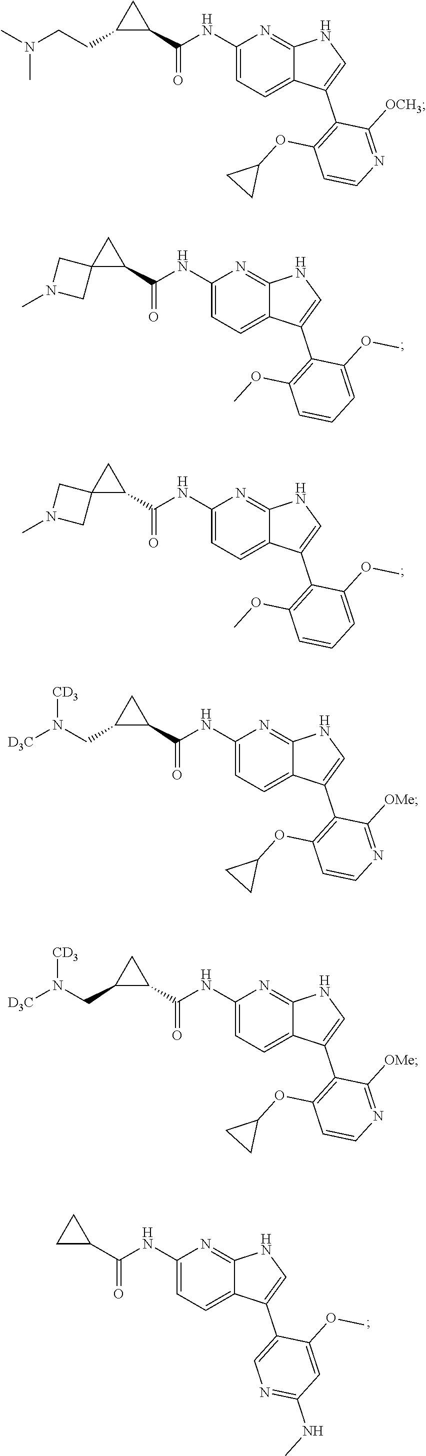 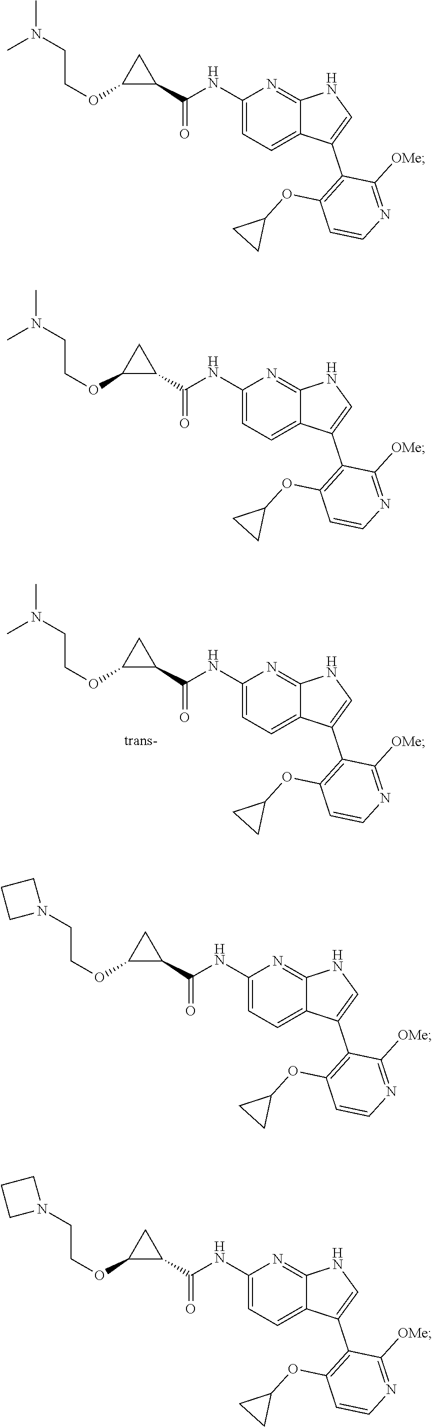 [SMILES: COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@H]3C[C@@H]3OCCN(C)C)ccc12 ; COc1nccc(OC2CC2)c1-c1c[nH]c2nc(NC(=O)[C@@H]3C[C@H]3OCCN(C)C)ccc12 ; COc1nccc(OC2CC2)c1-c1c[nH]c2nc(NC(=O)[C@@H]3C[C@H]3OCCN3CCC3)ccc12 ; COc1nccc(OC2CC2)c1-c1c[nH]c2nc(NC(=O)[C@H]3C[C@@H]3OCCN3CCC3)ccc12] 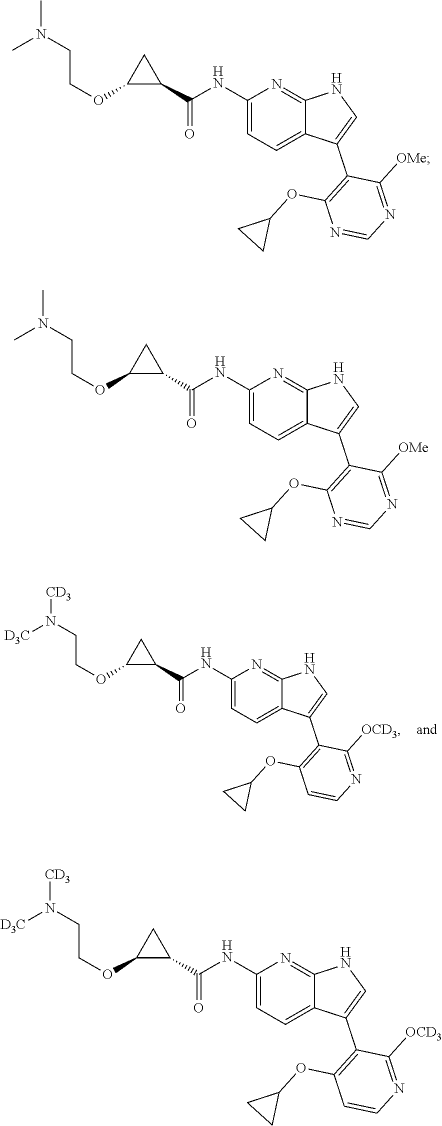
or a pharmaceutically acceptable salt, solvate, hydrate, or co-crystal thereof, or a mixture of any of the foregoing.

22. A pharmaceutical composition comprising the compound of claim 1, or a pharmaceutically acceptable salt, solvate, hydrate, or co-crystal thereof, or a mixture of any of the foregoing, and one or more pharmaceutically acceptable excipients.

23. (canceled)

24. A method of treating chronic myeloid leukemia (CML), acute myeloid leukemia (AML), acute lymphoblastic leukemia (ALL), or a mixed phenotype acute leukemia, in a human in need thereof, comprising administering to the human the compound of claim 1, or a pharmaceutically acceptable salt, solvate, hydrate, or co-crystal thereof, or a mixture of any of the foregoing.

25. The method of claim 24, wherein the leukemia is refractory leukemia.

26. The method of claim 25, wherein the refractory leukemia is associated with a mutation in the Bcr-Abl tyrosine kinase gene resulting in specific amino acid substitutions selected from the group consisting of M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I.

27. The method of claim 26, wherein the refractory leukemia is associated with a mutation in the Bcr-Abl tyrosine kinase gene resulting in specific amino acid substitution T315I.

28. The method of claim 25, wherein the human having refractory leukemia has one or more mutations in the Bcr-Abl tyrosine kinase gene resulting in specific amino acid substitutions selected from the group consisting of M244V, L248V, G250E, G250A, Q252H, Q252R, Y253F, Y253H, E255K, E255V, D276G, F311L, T315N, T315A, F317V, F317L, M343T, M351T, E355G, F359A, F359V, V379I, F382L, L387M, H396P, H396R, S417Y, E459K, F486S, and T315I.

29. The method of claim 28, wherein the human having refractory leukemia has a mutation in the Bcr-Abl tyrosine kinase gene resulting in specific amino acid substitution T315I.

30. (canceled)
